(19) United States
(12) Patent Application Publication
(10) Pub. No.: US 2026/0166183 A1
(43) Pub. Date: Jun. 18, 2026

(54) NEUROTENSIN RECEPTOR LIGANDS

(71) Applicant: ELI LILLY AND COMPANY, Indianapolis, IN (US)

(72) Inventors: Timothy Patrick CHOLKO, San Diego, CA (US); Shuai CHEN, San Diego, CA (US); Jeong Joo KIM, Broomfield, CO (US); Kyunga LEE, San Diego, CA (US); Zhiwei MA, San Diego, CA (US); Murali Mohan Reddy PERAM SURAKATTULA, San Diego, CA (US); Gerit Maria POTOTSCHNIG, San Diego, CA (US); Marcos Adrian SAINZ, San Diego, CA (US); Nathan Edward WRIGHT, San Diego, CA (US)

(21) Appl. No.: 19/421,728

(22) Filed: Dec. 16, 2025

Related U.S. Application Data
(60) Provisional application No. 63/735,007, filed on Dec. 17, 2024.
(60) Provisional application No. 63/905,149, filed on Oct. 24, 2025.

(51) Int. Cl. A61K 51/04; C07D 405/14; C07D 413/14; C07D 471/04; C07D 471/10; C07D 487/08; C07D 487/10; C07D 493/04; C07F 5/00

(52) U.S. Cl. CPC A61K 51/0497; C07D 405/14; C07D 413/14; C07D 471/04; C07D 471/10; C07D 487/08; C07D 487/10; C07D 493/04; C07F 5/003; A61K 2121/00

(57) ABSTRACT

The present invention provides a compound of the formula:

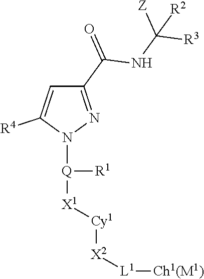

or a pharmaceutically acceptable salt thereof, wherein R¹, R², R³, R⁴, Z, Q, X¹, Cy¹, X², L¹, Ch¹, and M¹ are as defined herein. The present invention also provides pharmaceutical compositions including the compounds, methods of using the compounds for the treatment of disease, and methods of making the compounds.

### TECHNICAL FIELD

The present invention relates to neurotensin receptor ligands, pharmaceutical compositions including the neurotensin receptor ligands, methods of using the neurotensin receptor ligands for the treatment of disease, and methods of making the neurotensin receptor ligands.

### BACKGROUND

Neurotensin receptors (NTSRs) are G protein-coupled receptors (GPCRs) involved in regulation of the dopamine pathway and metabolism in the central nervous system. Neurotensin receptor 1 (NTSR1) is overexpressed in several cancers (e.g., breast, endometrial, gastric, colorectal, lung, pancreatic, and head and neck cancers). Small molecule agonists and antagonists have been developed and adapted into radioligand therapies (RLTs) targeting cancers associated with NTSR1 overexpression. Nonetheless, there exists a need for NTSR1-targeted RLTs with one or more of improved selectivity, potency, stability, biodistribution, or time on tumor.

### SUMMARY

In one aspect, the present invention provides a compound of Formula (I):

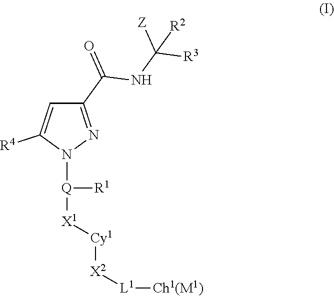

wherein,
R¹ is C₁-C₆ alkyl or C₃-C₆ cycloalkyl;
Z is —COOH, —C(═O)NH₂, or -tetrazolyl;
R² is C₆-C₁₀ cycloalkyl or C₆-C₁₀ cycloalkyl-C₁-C₆ alkyl-, wherein the C₆-C₁₀ cycloalkyl and C₆-C₁₀ cycloalkyl-C₁-C₆ alkyl- are each optionally substituted with 1-3 halogen;
R³ is H; or
R² and R³ together with the carbon atom to which they are attached form a C₆-C₁₀ cycloalkyl optionally substituted with 1-3 halogen;
R⁴ is

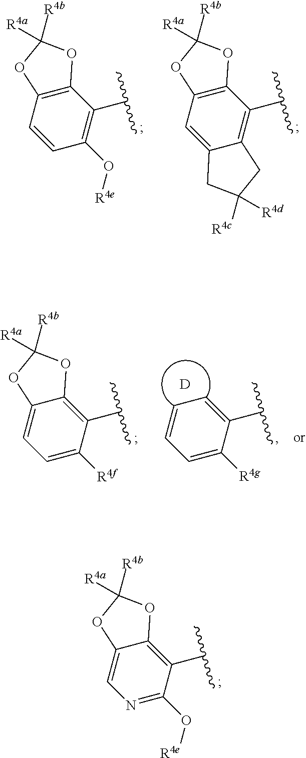

R^{4a}, R^{4b}, R^{4c}, and R^{4d} are each independently H or halogen;
R^{4e} is H, methyl, trifluoromethyl, or methoxyethyl;
R^{4f} is cyano or fluoro;
R^{4g} is H;
D is a 5- to 6-membered heteroaryl containing 1-3 heteroatoms independently selected from N, O, and S, optionally substituted with 1-3 groups independently selected from halo, hydroxy, amino, cyano, nitro, C₁-C₆ alkyl, C₁-C₆ haloalkyl, C₁-C₆ alkoxy, C₁-C₆ haloalkoxy; C₁-C₆ alkylamino, and C₁-C₆ dialkylamino;
Q is a 6-membered aryl or heteroaryl ring containing 0-3 ring heteroatoms independently selected from N, O, and S, the remaining ring atoms being carbon;
X¹ is a bond, —O—, or —NR^{x1}—;
Cy¹ is a bond or a 4- to 12-membered monocyclic or polycyclic ring system optionally substituted with 1-3 substituents independently selected from halo, hydroxy, amino, cyano, nitro, C₁-C₆ alkyl, C₁-C₆ haloalkyl, C₁-C₆ alkoxy, C₁-C₆ haloalkoxy, C₁-C₆ alkylamino, and C₁-C₆ dialkylamino, and optionally bearing 1-3 oxo substituents; provided that when Cy¹ is a bond, X¹ is a bond;
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—;
R^{x1} is H or C₁-C₃ alkyl;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
L¹ is a linker;
Ch¹ is a chelator; and
M¹ is absent or is a radionuclide complexed with Ch¹;
or a pharmaceutically acceptable salt thereof.

In another aspect, provided herein are methods of using the compounds of Formula (I), pharmaceutically acceptable salts thereof, and pharmaceutical compositions thereof, to treat cancer. The methods include administering a therapeutically effective amount of a compound of Formula (I), or a pharmaceutically acceptable salt thereof, to a patient in need thereof.

In another aspect, provided herein are compounds of Formula (I), and pharmaceutically acceptable salts thereof, for use in therapy. Additionally provided herein, are the compounds of Formula (I), and pharmaceutically acceptable salts thereof, for use in the treatment of cancer. Also provided herein is the use of compounds of Formula (I), or pharmaceutically acceptable salts thereof, in the manufacture of a medicament for treating cancer.

### DETAILED DESCRIPTION

The present invention provides a compound of Formula (I):

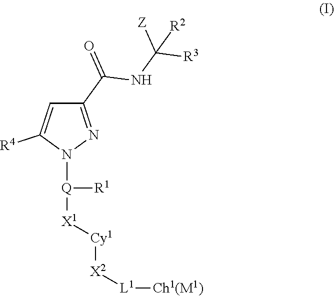

wherein R¹, R², R³, Z, R⁴, Q, X¹, Cy¹, X², L¹, Ch¹, and M¹ are as defined above, or a pharmaceutically acceptable salt thereof.

In an embodiment of a compound of Formula (I), or a pharmaceutically acceptable salt thereof, R¹ is C₁-C₆ alkyl or C₃-C₆ cycloalkyl. In an embodiment of a compound of Formula (I), or a pharmaceutically acceptable salt thereof, R¹ is isopropyl or tert-butyl. In an embodiment of a compound of Formula (I), or a pharmaceutically acceptable salt thereof, R¹ is isopropyl. In an embodiment of a compound of Formula (I), or a pharmaceutically acceptable salt thereof, R¹ is tert-butyl.

In an embodiment of a compound of Formula (I), or a pharmaceutically acceptable salt thereof, R² is C₆-C₁₀ cycloalkyl or C₆-C₁₀ cycloalkyl-C₁-C₆ alkyl-, wherein the C₆-C₁₀ cycloalkyl and C₆-C₁₀ cycloalkyl-C₁-C₆ alkyl- are each optionally substituted with 1-3 halogen and R³ is H. In an embodiment of a compound of Formula (I), or a pharmaceutically acceptable salt thereof, R² and R³ together with the carbon atom to which they are attached form a C₆-C₁₀ cycloalkyl optionally substituted with 1-3 halogen. In an embodiment of a compound of Formula (I), or a pharmaceutically acceptable salt thereof, R² is adamantyl or adamantyl-CH₂—, and R³ is H. In an embodiment of a compound of Formula (I), or a pharmaceutically acceptable salt thereof, R² is adamantyl, and R³ is H. In an embodiment of a compound of Formula (I), or a pharmaceutically acceptable salt thereof, R² is adamantyl-CH₂—, and R³ is H.

In an embodiment of a compound of Formula (I), or a pharmaceutically acceptable salt thereof, R² and R³ together with the carbon atom to which they are attached form an adamantyl.

In an embodiment of a compound of Formula (I), or a pharmaceutically acceptable salt thereof, Z is selected from —COOH, —C(═O)NH₂, and -tetrazolyl.

In an embodiment of a compound of Formula (I), or a pharmaceutically acceptable salt thereof, Z is —COOH. In an embodiment of a compound of Formula (I), or a pharmaceutically acceptable salt thereof, Z is —C(═O)NH₂. In an embodiment of a compound of Formula (I), or a pharmaceutically acceptable salt thereof, Z is -tetrazolyl.

In an embodiment of a compound of Formula (I), or a pharmaceutically acceptable salt thereof, R⁴ is selected from

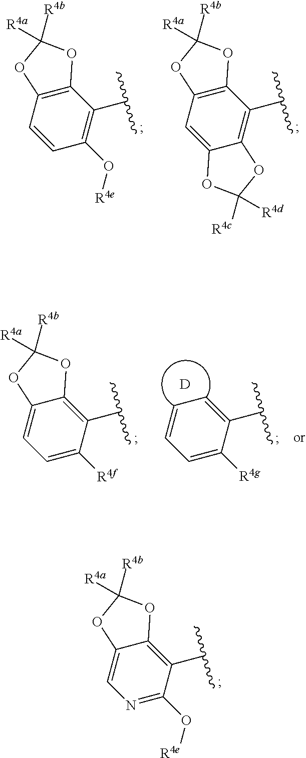

wherein R^{4a}, R^{4b}, R^{4c} and R^{4d} are each independently H or halogen.

In an embodiment of a compound of Formula (I), or a pharmaceutically acceptable salt thereof, D is a 5- to 6-membered heteroaryl containing 1-3 heteroatoms independently selected from N, O, and S, optionally substituted with 1-3 groups independently selected from halo, hydroxy, amino, cyano, nitro, C₁-C₆ alkyl, C₁-C₆ haloalkyl, C₁-C₆ alkoxy, C₁-C₆ haloalkoxy; C₁-C₆ alkylamino, or C₁-C₆ dialkylamino.

In an embodiment of a compound of Formula (I), or a pharmaceutically acceptable salt thereof, R⁴ is

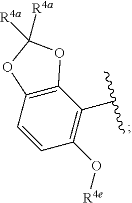

wherein R^{4a} and R^{4b} are each independently H or halogen (preferably fluoro).

In an embodiment of a compound of Formula (I), or a pharmaceutically acceptable salt thereof, R⁴ is

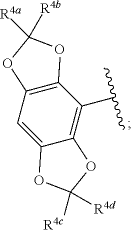

wherein R^{4a}, R^{4b}, R^{4c}, and R^{4d} are each independently H or halogen (preferably fluoro).

In an embodiment of a compound of Formula (I), or a pharmaceutically acceptable salt thereof, R⁴ is

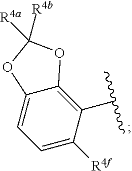

wherein R^{4a} and R^{4b} are each independently H or halogen (preferably fluoro).

In an embodiment of a compound of Formula (I), or a pharmaceutically acceptable salt thereof, R⁴ is

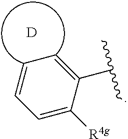

In an embodiment of a compound of Formula (I), or a pharmaceutically acceptable salt thereof. R⁴ is

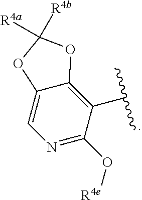

In an embodiment of a compound of Formula (I), or a pharmaceutically acceptable salt thereof, R⁴ is selected from:

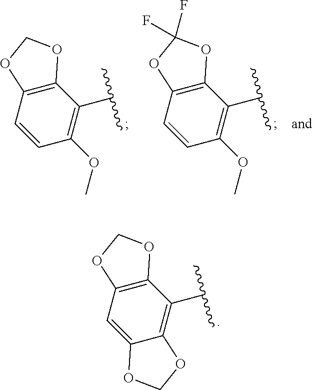 [SMILES: *c1c(OC)ccc2c1OCO2 ; *c1c(OC)ccc2c1OC(F)(F)O2 ; *c1c2c(cc3c1OCO3)OCO2]

In an embodiment of a compound of Formula (I), or a pharmaceutically acceptable salt thereof, R⁴ is

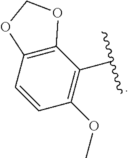 [SMILES: *c1c(OC)ccc2c1OCO2]

In an embodiment of a compound of Formula (I), or a pharmaceutically acceptable salt thereof, R⁴ is

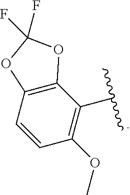 [SMILES: *c1c(OC)ccc2c1OC(F)(F)O2]

In an embodiment of a compound of Formula (I), or a pharmaceutically acceptable salt thereof, R⁴ is

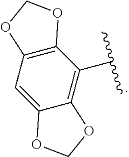 [SMILES: *c1c2c(cc3c1OCO3)OCO2]

In an embodiment of a compound of Formula (I), or a pharmaceutically acceptable salt thereof, R⁴ is 5-methoxybenzo[d][1,3]dioxol-4-yl; 2,2-difluoro-5-methoxybenzo[d][1,3]dioxol-4-yl; or benzo[1,2-d:4,5-d′]bis([1,3]dioxole)-4-yl. In an embodiment of a compound of Formula (I), or a pharmaceutically acceptable salt thereof, R⁴ is 5-methoxybenzo[d][1,3]dioxol-4-yl.

In an embodiment of a compound of Formula (I), or a pharmaceutically acceptable salt thereof, R⁴ is

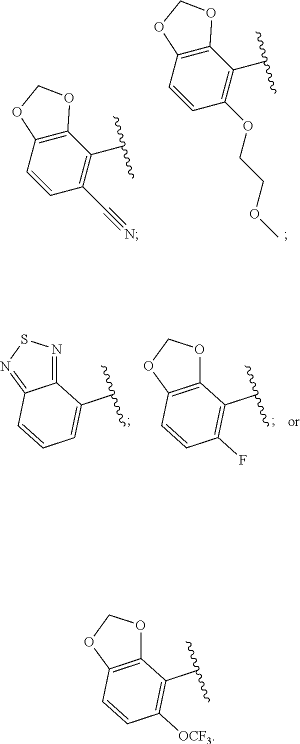

In an embodiment of a compound of Formula (I), or a pharmaceutically acceptable salt thereof, R⁴ is

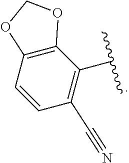 [SMILES: *c1c(C#N)ccc2c1OCO2]

In an embodiment of a compound of Formula (I), or a pharmaceutically acceptable salt thereof, R⁴ is

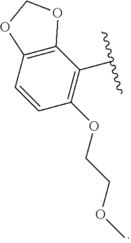 [SMILES: *c1c(OCCOC)ccc2c1OCO2]

In an embodiment of a compound of Formula (I), or a pharmaceutically acceptable salt thereof, R⁴ is

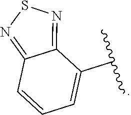 [SMILES: *c1cccc2nsnc12]

In an embodiment of a compound of Formula (I), or a pharmaceutically acceptable salt thereof, R⁴ is

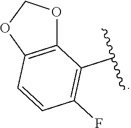 [SMILES: *c1c(F)ccc2c1OCO2]

In an embodiment of a compound of Formula (I), or a pharmaceutically acceptable salt thereof, R⁴ is

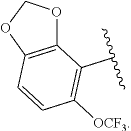 [SMILES: *c1c(OC(F)(F)F)ccc2c1OCO2]

In an embodiment of a compound of Formula (I), or a pharmaceutically acceptable salt thereof, R⁴ is

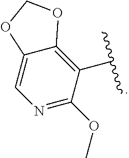 [SMILES: *c1c(OC)ncc2c1OCO2]

In an embodiment of a compound of Formula (I), or a pharmaceutically acceptable salt thereof, R⁴ is 5-cyanobenzo[d][1,3]dioxol-4-yl; 5-(2-methoxyethoxybenzo[d][1,3]dioxol-4-yl; benzo[c]]1,2,5]thiadiazol-4-yl; 5-fluoro[d][1,3]dioxol-4-yl; or 5-trifluoromethoxy[d][1,3]dioxol-4-yl.

In an embodiment of a compound of Formula (I), or a pharmaceutically acceptable salt thereof, Q is a 6-membered aryl or heteroaryl ring containing 0-3 ring heteroatoms independently selected from N, O, and S, the remaining ring atoms being carbon.

In an embodiment of a compound of Formula (I), or a pharmaceutically acceptable salt thereof, Q is

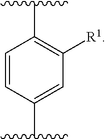

In an embodiment of a compound of Formula (I), or a pharmaceutically acceptable salt thereof, Q is

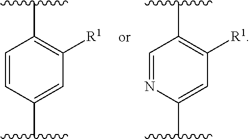

In an embodiment of a compound of Formula (I), or a pharmaceutically acceptable salt thereof, Q is

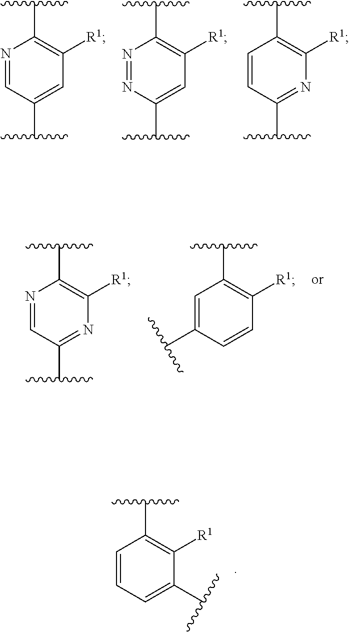

In an embodiment of a compound of Formula (I), or a pharmaceutically acceptable salt thereof, R⁴ is

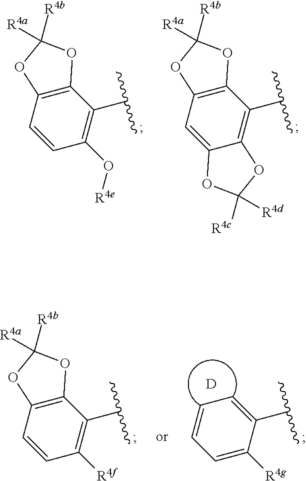

and

### Q is

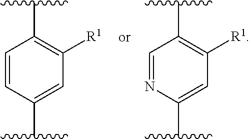

In an embodiment of a compound of Formula (I), or a pharmaceutically acceptable salt thereof, X¹ is —NR^{x1}—, wherein R^{x1} is H or C₁-C₃ alkyl. In an embodiment of a compound of Formula (I), or a pharmaceutically acceptable salt thereof, X¹ is a bond. In an embodiment of a compound of Formula (I), or a pharmaceutically acceptable salt thereof, X¹ is —O—. In an embodiment of a compound of Formula (I), or a pharmaceutically acceptable salt thereof, X¹ is —NH—.

In an embodiment of a compound of Formula (I), or a pharmaceutically acceptable salt thereof, Cy¹ is a bond or a 4- to 12-membered monocyclic or polycyclic ring system optionally substituted with 1-3 substituents independently selected from halo, hydroxy, amino, cyano, nitro, C₁-C₆ alkyl, C₁-C₆ haloalkyl, C₁-C₆ alkoxy, C₁-C₆ haloalkoxy, C₁-C₆ alkylamino, and C₁-C₆ dialkylamino, and optionally bearing 1-3 oxo substituents; provided that when Cy¹ is a bond, X¹ is a bond.

In an embodiment of a compound of Formula (I), or a pharmaceutically acceptable salt thereof, Cy¹ is a bond, phenyl, a 5- or 6-membered heteroaryl containing 1-3 heteroatoms independently selected from N, O, and S, a C₄-C₆ cycloalkyl, a C₄-C₆ cycloalkenyl, a 4- to 6-membered heterocycloalkyl containing 1-3 heteroatoms independently selected from N, O, and S, or a 4- to 6-membered heterocycloalkenyl containing 1-3 heteroatoms independently selected from N, O, and S; wherein the phenyl, heteroaryl, cycloalkyl, cycloalkenyl, heterocycloalkyl, or heterocycloalkenyl is optionally fused to a ring selected from phenyl, a 5- or 6-membered heteroaryl, a C₄-C₆ cycloalkyl, a C₄-C₆ cycloalkenyl, a 4- to 6-membered heterocycloalkyl containing 1-3 heteroatoms independently selected from N, O, and S, and a 4- to 6-membered heterocycloalkenyl; or wherein the cycloalkyl, cycloalkenyl, heterocycloalkyl or heterocycloalkenyl is optionally spiro-connected to another ring selected from a C₄-C₆ cycloalkyl, a C₄-C₆ cycloalkenyl, a 4- to 6-membered heterocycloalkyl containing 1-3 heteroatoms independently selected from N, O, and S, or a 4- to 6-membered heterocycloalkenyl containing 1-3 heteroatoms independently selected from N, O, and S; or wherein the cycloalkyl, cycloalkenyl, heterocycloalkyl or heterocycloalkenyl optionally comprises a bridging group having 1, 2, or 3 bridging atoms independently selected from N, O, and S; and wherein Cy¹ is optionally substituted with 1-3 substituents independently selected from the group consisting of halo, hydroxy, amino, cyano, nitro, C₁-C₆ alkyl, C₁-C₆ haloalkyl, C₁-C₆ alkoxy, C₁-C₆ haloalkoxy, C₁-C₆ alkylamino, and C₁-C₆ dialkylamino, and optionally bears 1-3 oxo substituents; provided that when Cy¹ is a bond, X¹ is a bond.

In an embodiment of a compound of Formula (I), or a pharmaceutically acceptable salt thereof, Cy¹ is a bond, phenyl, or a triazolyl. In an embodiment of a compound of Formula (I), or a pharmaceutically acceptable salt thereof, Cy¹ is a bond. In an embodiment of a compound of Formula (I), or a pharmaceutically acceptable salt thereof, Cy¹ is phenyl. In an embodiment of a compound of Formula (I), or a pharmaceutically acceptable salt thereof, Cy¹ is triazolyl.

In an embodiment of a compound of Formula (I), or a pharmaceutically acceptable salt thereof, Cy¹ is a 1,3-difluorophenyl.

In an embodiment of a compound of Formula (I), or a pharmaceutically acceptable salt thereof, Cy¹ is a monocyclic heteroaryl ring that is 1,2,3-triazolyl; 1,3,4-thiadiazolyl; pyridine; or pyridazine.

In an embodiment of a compound of Formula (I), or a pharmaceutically acceptable salt thereof, Cy¹ is a monocyclic heteroaryl ring that is 3-fluoropyridine.

In an embodiment of a compound of Formula (I), or a pharmaceutically acceptable salt thereof, Cy¹ is a fused bicyclic ring system that is isoindolin-1-one; isoquinolin-1-one; 3,4-dihydroisoquinolin-1-one; isoquinoline; 3,4-dihydroisoquinoline; quinoline; indazole; or naphthalene.

In an embodiment of a compound of Formula (I), or a pharmaceutically acceptable salt thereof, Cy¹ is a fused bicyclic ring system that is 1,5-naphthyridine; quinoxaline; or [1,2,3]triazolo[1,5-a]pyridine.

In an embodiment of a compound of Formula (I), or a pharmaceutically acceptable salt thereof, Cy¹ is a saturated or partially saturated heterocyclic ring (including spirocyclic moieties) that is piperidine; piperazine; 3,6-dihydropyridine; 3-azaspiro[5.5]undecane; or 3,9-diazaspiro[5.5]undecane.

In an embodiment of a compound of Formula (I), or a pharmaceutically acceptable salt thereof, Cy¹ is a saturated or partially saturated heterocyclic ring (including spirocyclic and bridged moieties) that is 2,5-diazabicyclo[2.2.1]heptane; 2,6-diazaspiro[3.3]heptane; 2,7-diazaspiro[3.5]nonane; or 3,9-diazaspiro[5.5]undecan-2-one.

In an embodiment of a compound of Formula (I), or a pharmaceutically acceptable salt thereof, Cy¹ is an aromatic ring substituted with an electron-withdrawing or electron-donating group, that is a benzonitrile ring (a cyano-substituted phenyl ring); a nitrophenyl ring; or a methoxyphenyl ring.

In an embodiment of a compound of Formula (I), or a pharmaceutically acceptable salt thereof, Cy¹ is

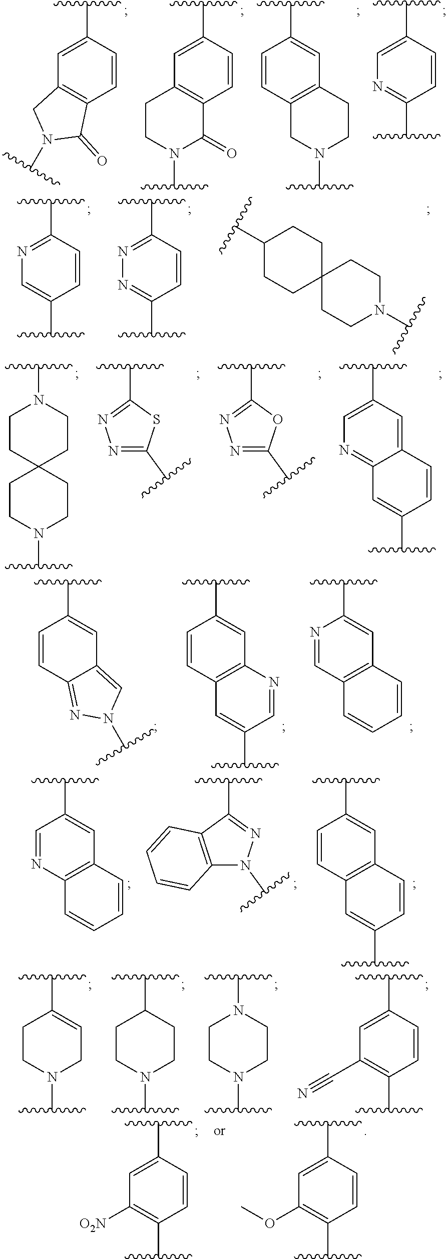

In an embodiment of a compound of Formula (I), or a pharmaceutically acceptable salt thereof, Cy¹ is

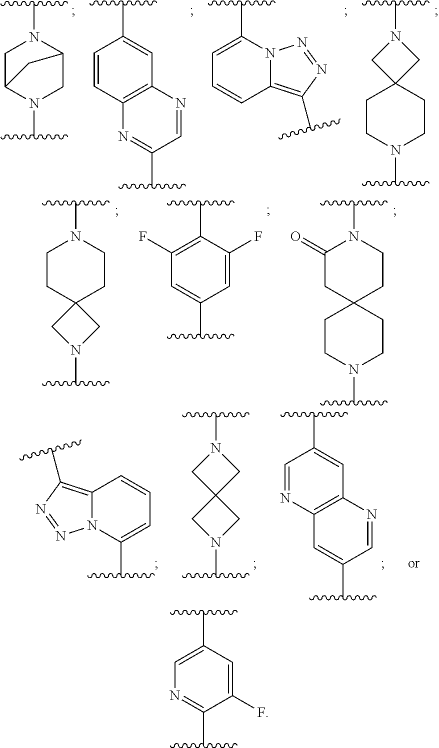

In an embodiment of a compound of Formula (I), or a pharmaceutically acceptable salt thereof Z is —COOH; R⁴ is

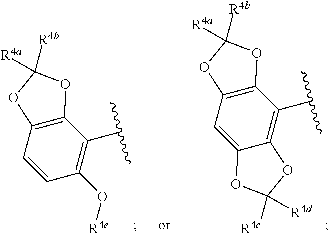

is R^{4e} is methyl; and Cy¹ is phenyl or a 5- or 6-membered heteroaryl containing 1-3 heteroatoms independently selected from N, O, and S (provided that when Cy¹ is a bond, X¹ is a bond).

In an embodiment of a compound of Formula (I), or a pharmaceutically acceptable salt thereof, X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—; and R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a pyrrolidinyl, wherein the pyrrolidinyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;

In an embodiment of a compound of Formula (I), or a pharmaceutically acceptable salt thereof, X² is a bond, —NR^{x2}—, —O—, —NR^{x3}C(O)—, —C(O)NR^{x5}—, —NR^{x7}C(O)NR^{x8}—, —NR^{x11}C(O)O—, —NR^{x13}C(O)S—, or —NR^{x19}S(O)₂NR^{x20}—, R^{x2}, R^{x3}, R^{x5}, R^{x7}, R^{x8}, R^{x11}, R^{x13}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x2}, R^{x3}, R^{x5}, R^{x7}, R^{x8}, R^{x11}, R^{x13}, R^{x19}, and R^{x20} are each independently selected from H and C₁-C₆ alkyl, more preferably R^{x2}, R^{x3}, R^{x5}, R^{x7}, R^{x8}, R^{x11}, R^{x13}, R^{x19}, and R^{x20} are each independently selected from H and methyl.

In an embodiment of a compound of Formula (I), or a pharmaceutically acceptable salt thereof, X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—.

In an embodiment of a compound of Formula (I), or a pharmaceutically acceptable salt thereof, X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—.

In an embodiment of a compound of Formula (I), or a pharmaceutically acceptable salt thereof, X² is a bond. In an embodiment of a compound of Formula (I), or a pharmaceutically acceptable salt thereof, X² is —NH—. In an embodiment of a compound of Formula (I), or a pharmaceutically acceptable salt thereof, X² is —NCH₃—. In an embodiment of a compound of Formula (I), or a pharmaceutically acceptable salt thereof, X² is —O—. In an embodiment of a compound of Formula (I), or a pharmaceutically acceptable salt thereof, X² is —NHC(O)—. In an embodiment of a compound of Formula (I), or a pharmaceutically acceptable salt thereof, X² is —NCH₃C(O)—. In an embodiment of a compound of Formula (I), or a pharmaceutically acceptable salt thereof, X² is —C(O)NCH₃—. In an embodiment of a compound of Formula (I), or a pharmaceutically acceptable salt thereof, X² is —NHC(O)NH—. In an embodiment of a compound of Formula (I), or a pharmaceutically acceptable salt thereof, X² is —NHC(O)NCH₃—. In an embodiment of a compound of Formula (I), or a pharmaceutically acceptable salt thereof, X² is —NCH₃C(O)NCH₃—. In an embodiment of a compound of Formula (I), or a pharmaceutically acceptable salt thereof, X² is —NHC(O)O—. In an embodiment of a compound of Formula (I), or a pharmaceutically acceptable salt thereof, X² is —NCH₃C(O)O—. In an embodiment of a compound of Formula (I), or a pharmaceutically acceptable salt thereof, X² is —NHC(O)S—. In an embodiment of a compound of Formula (I), or a pharmaceutically acceptable salt thereof, X² is —NHS(O)₂NCH₃—.

In an embodiment of a compound of Formula (I), or a pharmaceutically acceptable salt thereof, L¹ includes one or more groups independently selected from:

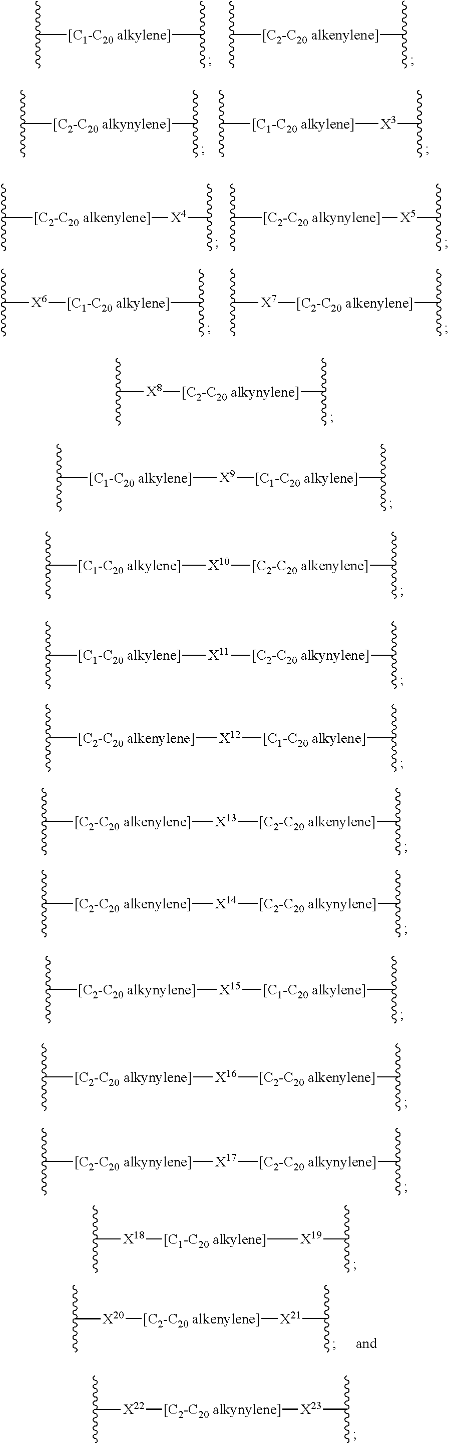

wherein
X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ at each occurrence are independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₂₀ alkylene, C₂-C₂₀ alkenylene, and C₂-C₂₀ alkynylene at each occurrence are optionally interrupted by one or more groups independently selected from C₃-C₁₀ cycloalkylene, C₄-C₁₀ heterocycloalkylene, C₆-C₁₀ arylene, and C₅-C₁₀ heteroarylene; and
C₁-C₂₀ alkylene, C₁-C₂₀ alkenylene, C₁-C₂₀ alkynylene, C₃-C₁₀ cycloalkylene, C₄-C₁₀ heterocycloalkylene, C₆-C₁₀ arylene, and C₅-C₁₀ heteroarylene at each occurrence are optionally substituted with one or more groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with one or more groups independently selected from halo, —CO₂H, —OH, —NH₂, —NHC₁-C₄ alkyl, and —N(C₁-C₄ alkyl)(C₁-C₄ alkyl).

As used herein with respect to an alkylene group, for example a C₁-C₂₀ alkylene, “optionally interrupted by 1-5 groups” means that the alkylene backbone may comprise one or more alkylene segments separated by zero, one, two, three, four, or five interrupting units as expressly recited for the relevant embodiment, for example a C₃-C₁₀ cycloalkylene, C₄-C₁₀ heterocycloalkylene, C₆-C₁₀ arylene, or C₅-C₁₀ heteroarylene. Each interrupting unit is a divalent residue that is bonded at two positions to the adjacent alkylene carbon atoms such that it forms part of the L¹ backbone and is inserted into the backbone rather than pendant from it. Unless indicated otherwise, the stated carbon count for the alkylene group, for example “C₁-C₂₀,” refers to the total number of carbon atoms in the alkylene segments only and excludes atoms contained within any interrupting unit or units. The interrupting units may be the same or different and may occur in any order.

In an embodiment of a compound of Formula (I), or a pharmaceutically acceptable salt thereof, linker L¹ includes 1-20 groups independently selected from:

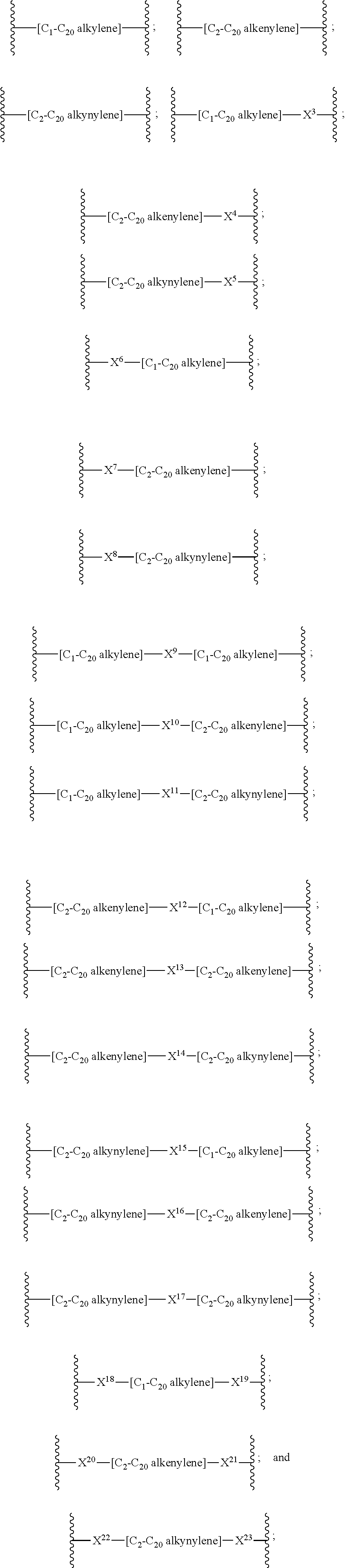

wherein
X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ at each occurrence are independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₂₀ alkylene, C₂-C₂₀ alkenylene, and C₂-C₂₀ alkynylene at each occurrence are optionally interrupted by 1-5 groups independently selected from C₃-C₁₀ cycloalkylene, C₄-C₁₀ heterocycloalkylene, C₆-C₁₀ arylene, and C₅-C₁₀ heteroarylene; and
C₁-C₂₀ alkylene, C₂-C₂₀ alkenylene, C₂-C₂₀ alkynylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl).

In an embodiment of a compound of Formula (I), or a pharmaceutically acceptable salt thereof, linker L¹ includes 1-5 groups independently selected from:

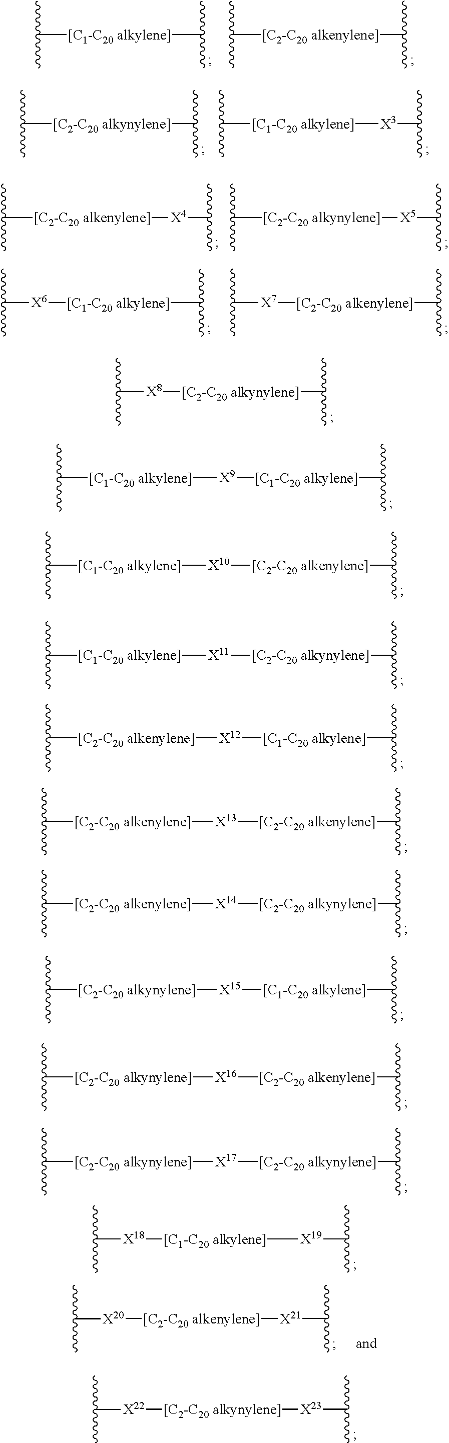

wherein
X³, X⁴, X⁵, X⁶, X¹, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ at each occurrence are independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}, —C(S)NR^{x25}, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₂₀ alkylene, C₂-C₂₀ alkenylene, and C₂-C₂₀ alkynylene at each occurrence are optionally interrupted by 1-5 groups independently selected from C₃-C₁₀ cycloalkylene, C₄-C₁₀ heterocycloalkylene, C₆-C₁₀ arylene, and C₅-C₁₀ heteroarylene; and
C₁-C₂₀ alkylene, C₂-C₂₀ alkenylene, C₂-C₂₀ alkynylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl).

In an embodiment of a compound of Formula (I), or a pharmaceutically acceptable salt thereof, L¹ includes 1-20 groups, preferably 1-5 groups, independently selected from

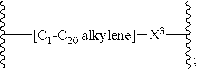

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}, —C(S)NR^{x25}, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}, —OC(S)NR^{x34}—, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₂₀ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene; and
C₁-C₂₀ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl).

In an embodiment of a compound of Formula (I), or a pharmaceutically acceptable salt thereof, L¹ includes 1-20 groups, preferably 1-5 groups, independently selected from

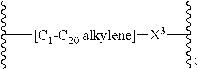

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₃ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene; and
C₁-C₃ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl).

In an embodiment of a compound of Formula (I), or a pharmaceutically acceptable salt thereof, L¹ includes 1-20 groups, preferably 1-5 groups, independently selected from

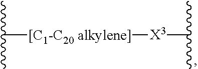

wherein X³ at each occurrence is independently selected from —NR^{x21}—, —O—, and —NR^{x22}C(O)—, and R^{x21} and R^{x22} at each occurrence are independently selected from H and C₁-C₆ alkyl.

In an embodiment of a compound of Formula (I), or a pharmaceutically acceptable salt thereof, L¹ includes 1-20 groups, preferably 1-5 groups, independently selected from

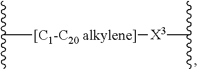

wherein X³ at each occurrence is independently selected from —NR^{x21}—, —O—, and —NR^{x22}C(O)—, and R^{x21} and R^{x22} at each occurrence are independently selected from H and C₁-C₆ alkyl.

In an embodiment of a compound of Formula (I), or a pharmaceutically acceptable salt thereof, L¹ includes 1-20 groups, preferably 1-5 groups, independently selected from

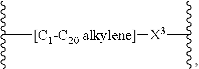

wherein X³ at each occurrence is independently selected from —NH—, —NCH₃—, —O—, and —NHC(O)—.

In an embodiment of a compound of Formula (I), or a pharmaceutically acceptable salt thereof, L¹ includes 1-20 groups, preferably 1-5 groups, independently selected from

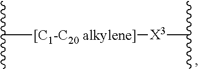

wherein X³ at each occurrence is independently selected from —NH—, —NCH₃—, —O—, and —NHC(O)—.

In an embodiment of a compound of Formula (I), or a pharmaceutically acceptable salt thereof, L¹ is

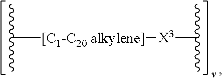

wherein v is 1, 2, 3, 4, or 5,
(i.e., L¹ is selected from

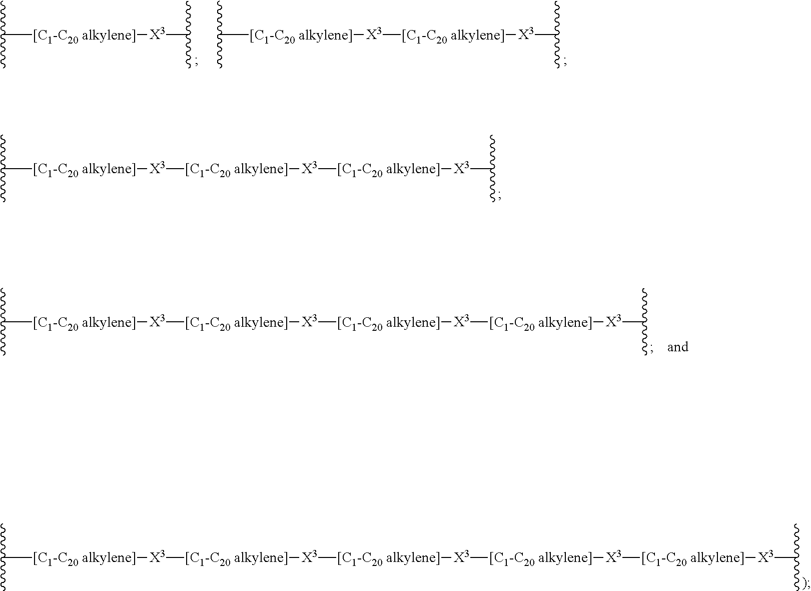

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}, —C(S)NR^{x25}, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₂₀ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene; and
C₁-C₂₀ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl).

In an embodiment of a compound of Formula (I), or a pharmaceutically acceptable salt thereof, L¹ is

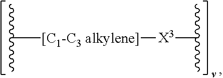

wherein v is 1, 2, 3, 4, or 5,
(i.e., L¹ is selected from

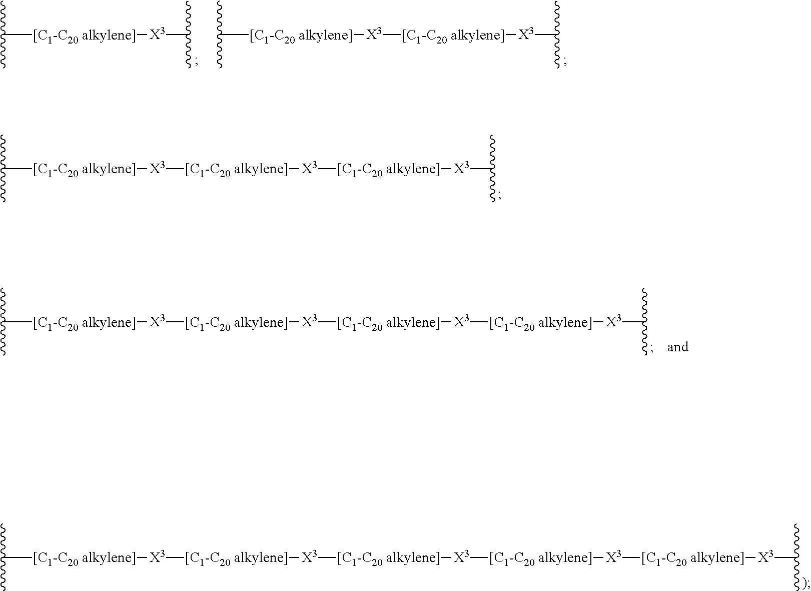

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₃ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene; and
C₁-C₃ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl).

In an embodiment of a compound of Formula (I), or a pharmaceutically acceptable salt thereof, L¹ is

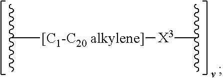

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5.

In an embodiment of a compound of Formula (I), or a pharmaceutically acceptable salt thereof, L¹ is

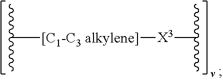

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5.

In an embodiment of a compound of Formula (I), or a pharmaceutically acceptable salt thereof, L¹ is

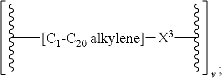

wherein X³ at each occurrence is independently selected from —NH—, —NCH₃—, —O—, and —NHC(O)—, and v is 1, 2, 3, 4, or 5.

In an embodiment of a compound of Formula (I), or a pharmaceutically acceptable salt thereof, L¹ is

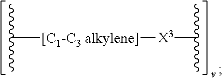

wherein X³ at each occurrence is independently selected from —NH—, —NCH₃—, —O—, and —NHC(O)—, and v is 1, 2, 3, 4, or 5.

In an embodiment of a compound of Formula (I), or a pharmaceutically acceptable salt thereof, L¹ is selected from

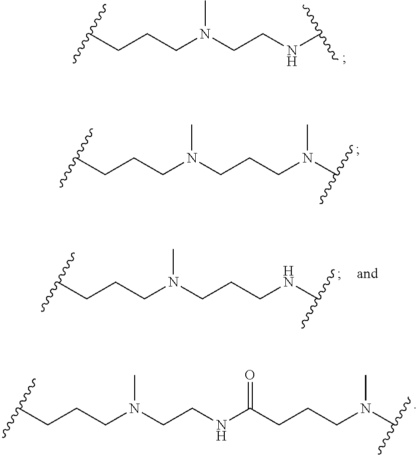

In an embodiment of a compound of Formula (I), or a pharmaceutically acceptable salt thereof, L¹ is

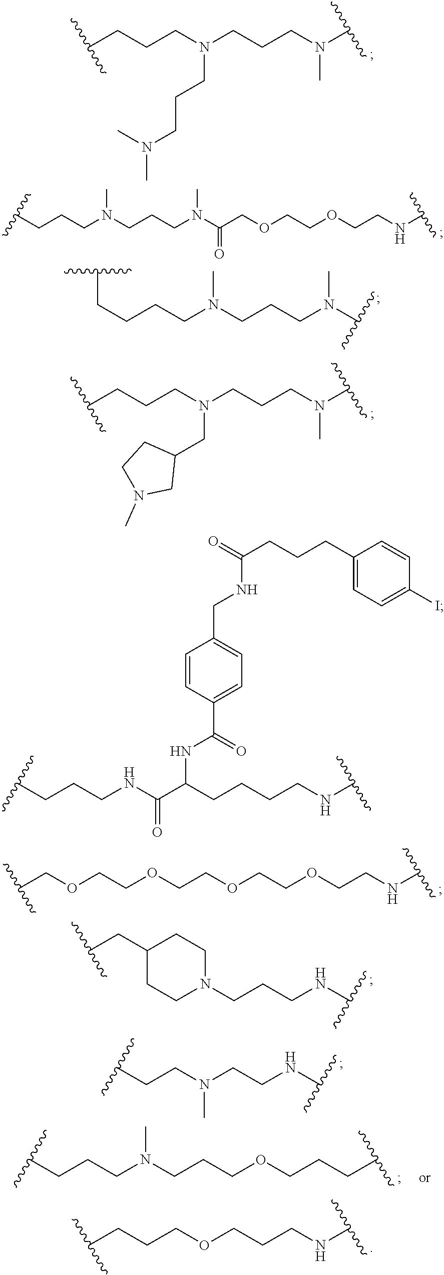

In an embodiment of a compound of Formula (I), or a pharmaceutically acceptable salt thereof, L¹ is

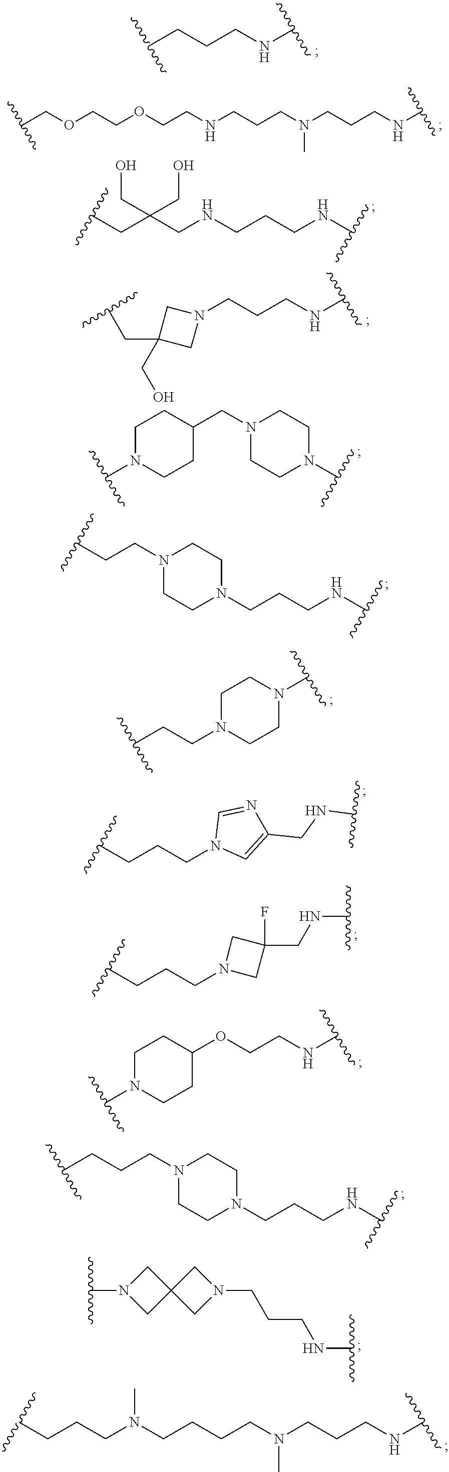

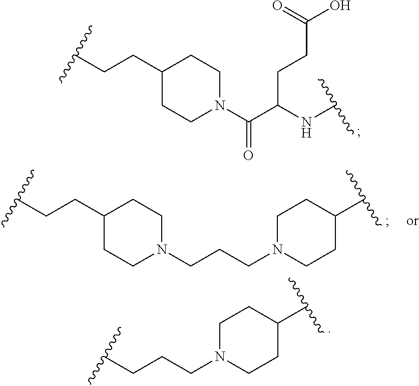

In an embodiment of a compound of Formula (I), or a pharmaceutically acceptable salt thereof, Ch¹ is derived from 4,7-triazacyclononane (TACN); 1,4,7-triazacyclononane-triacetic acid (NOTA); 1,4,7-triazacyclononane-N-succinic acid-N′,N″-diacetic acid (NOTASA); 1,4,7-triazacyclononane-N-glutamic acid-N′,N″-diacetic acid (NODAGA); 1,4,7-triazacyclononane-N,N′,N″-tris(methylenephosphonic) acid (NOTP); 1,4,7,10-tetraazacyclododecane ([12]aneN4) (cyclen); 1,4,7,10-tetraazacyclotridecane ([13]aneN4); 1,4,7,11-tetraazacyclotetradecane (iso-cyclam); 1,4,7,10-tetraazacyclododecane-1,4,7,10-tetraacetic acid (DOTA); 2-(1,4,7,10-tetraazacyclododecan-1-yl) acetate (DO1A); 2,2′-(1,4,7,10-tetraazacyclododecane-1,7-diyl) diacetic acid (DO2A); 2,2′,2″-(1,4,7,10-tetraazacyclododecane-1,4,7-triyl)triacetic acid (DO3A); 1,4,7,10-tetraazacyclododecane-1,4,7,10-tetra(methanephosphonic acid) (DOTP); 1,4,7,10-tetraazacyclododecane-1,7-di(methanephosphoric acid) (DO2P); 1,4,7,10-tetraazacyclododecane-1,4,7-tri(methanephosphonic acid) (DO3P); 1,4,7,10-tetraazacyclo-decane-1-glutamic acid-4,7,10-triacetic acid (DOTAGA); 1,4,7,10-tetraazacyclodecane-1-succinic acid-4,7,10-triacetic acid (DOTASA); 1,4,8,11-tetraazacyclotetradecane ([14]aneN4) (cyclam); 1,4,8,12-tetraazacyclopentadecane ([15]aneN4); 1,5,9,13-tetraazacyclohexadecane ([16]aneN4); 1,4-ethano-1,4,8,11-tetraazacyclo-tetradecane (et-cyclam); 1,4,8,11-tetraazacyclotetradecane-1,4,8,11-tetraacetic acid (TETA); 2-(1,4,8,11-tetraazacyclotetradecane-1-yl) acetic acid (TE1A); 2,2′-(1,4,8,11-tetraazacyclotetradecane-1,8-di-yl) diacetic acid (TE2A); 4,11-bis(carboxy methyl)-1,4,8,11-tetraazabicyclo[6.6.2]-hexadecane (CB-TE2A); 3,6,10,13,16,19-hexaazabicyclo[6.6.6]icosane (Sar); 2,2,2,2-(1,4,7,10-tetraazacyclododecane-1,4,7,10-tetraacetamide (TCMC); 1,4,7,10-tetraazacyclododecane-7-acetamide-1,4,10-triacetic acid (PSC); N,N′-bis[(6-carboxy-2-pyridil)methyl]-4,13-diaza-18-crown-6 (macropa), a phthalocyanine, or a porphoryin.

In an embodiment of a compound of Formula (I), or a pharmaceutically acceptable salt thereof, Ch¹ is derived from DOTA, DOTATOC, DO3A, PEPA, EDTA, CHX-A″-DTPA, HEHA, DOTMP, tu-BU-calix, arene-tetracarboxylic acid, macropa, macropa-NCS, H₄py₄pa, H₄octapa, H₄noonpa, H₅decapa, H₄CHXoctapa, DOTP, DOTPH, DOTPOEt, DOTPI, NETA, diethylenetriaminepentaacetic acid (DTPA), TCMC, 2-(4-isothiocyanatobenzyl)-6-methyl-diethylenetriaminepentaacetic acid (1B4M-DTPA), DTMP, HOPO, octapa me-3,2-HOPO, DOTANOC, DOTAMTATE, DATA^{5m}, PA-DOTA, DO4S, DO3S, DO2A2S, DTPAm, EGTA, EGTAm, H₂ampa, H₂ampa, H₂macropa, HEHA, py-macrodipa, DTCBP, or ATSM.

In an embodiment of a compound of Formula (I), or a pharmaceutically acceptable salt thereof, Ch¹ is an acyclic chelator. In an embodiment of a compound of Formula (I), or a pharmaceutically acceptable salt thereof, Ch¹ is a macrocyclic chelator.

In an embodiment of a compound of Formula (I), or a pharmaceutically acceptable salt thereof, Ch¹ is an aminopolycarboxylate chelator. In an embodiment of a compound of Formula (I), or a pharmaceutically acceptable salt thereof, Ch¹ is a macrocyclic aminopolycarboxylate chelator.

In an embodiment of a compound of Formula (I), or a pharmaceutically acceptable salt thereof, Ch¹ is selected from:

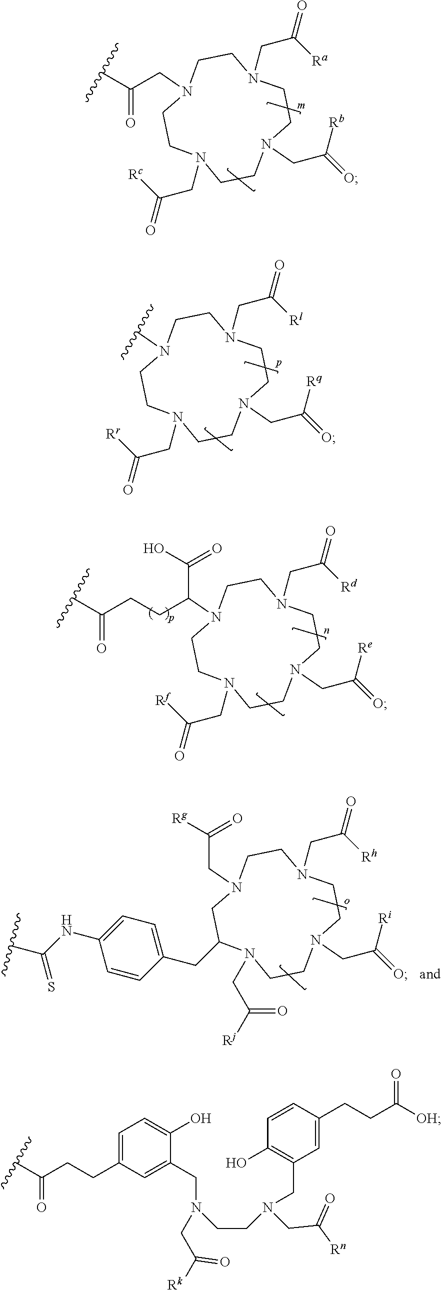

wherein R^{a}, R^{b}, R^{c}, R^{d}, R^{e}, R^{f}, R^{g}, R^{h}, Rⁱ, R^{j}, R^{k}, Rⁿ, R^{l}, R^{q}, and R^{r} are each independently selected from —OH and —NH₂; and m, n, o, and p are each independently 0, 1, or 2.

In an embodiment of a compound of Formula (I), or a pharmaceutically acceptable salt thereof, Ch¹ is selected from:

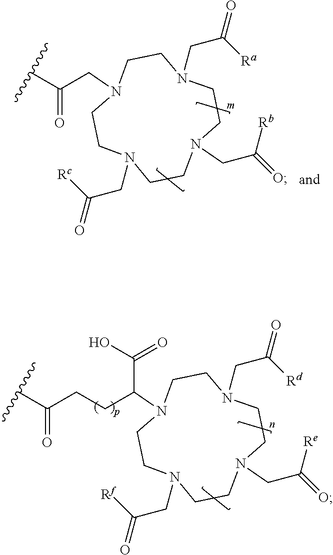

wherein R^{a}, R^{b}, R^{c}, R^{d}, R^{e}, and R^{f} are each independently selected from —OH and —NH₂; and m, n, and p are each independently 0, 1, or 2.

In an embodiment of a compound of Formula (I), or a pharmaceutically acceptable salt thereof, Ch¹ is:

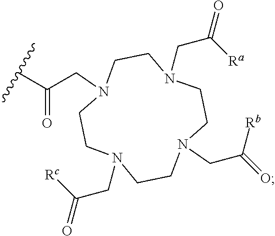

wherein R^{a}, R^{b}, and R^{c} are each independently selected from —OH and —NH₂. In an embodiment of a compound of Formula (I), or a pharmaceutically acceptable salt thereof, R^{a}, R^{b}, and R^{c} are each —OH. In an embodiment of a compound of Formula (I), or a pharmaceutically acceptable salt thereof, R^{a} and R^{c} are each —OH, and R^{b} is —NH₂. In an embodiment of a compound of Formula (I), or a pharmaceutically acceptable salt thereof, R^{a}, R^{b}, and R^{c} are each —NH₂.

In an embodiment of a compound of Formula (I), or a pharmaceutically acceptable salt thereof, Ch¹ is selected from:

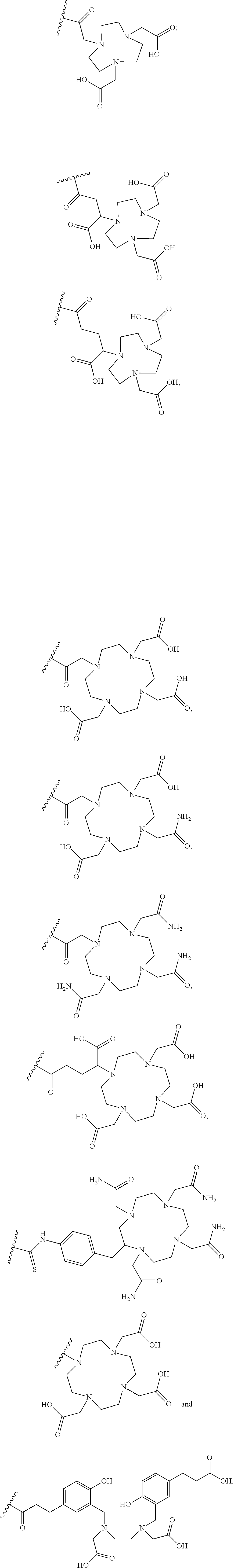

In an embodiment of a compound of Formula (I), or a pharmaceutically acceptable salt thereof, Ch¹ is selected from:

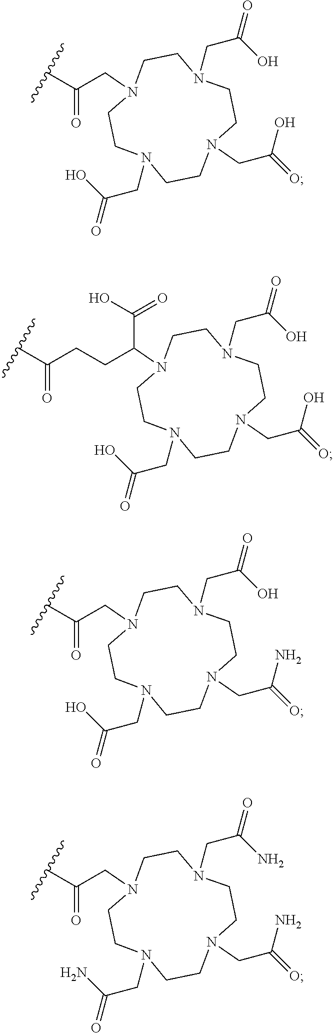 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1 ; *C(=O)CN1CCN(CC(=O)O)CCN(CC(N)=O)CCN(CC(=O)O)CC1 ; *C(=O)CN1CCN(CC(N)=O)CCN(CC(N)=O)CCN(CC(N)=O)CC1]

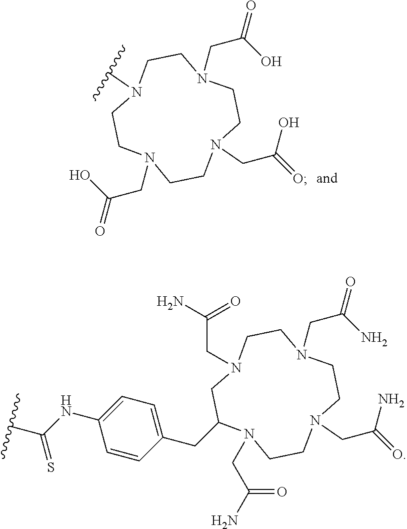 [SMILES: *N1CCN(CC(=O)O)CCN(CC(=O)O)CCN(CC(=O)O)CC1 ; *C(=S)Nc1ccc(CC2CN(CC(N)=O)CCN(CC(N)=O)CCN(CC(N)=O)CCN2CC(N)=O)cc1]

In an embodiment of a compound of Formula (I), or a pharmaceutically acceptable salt thereof, Ch¹ is selected from:

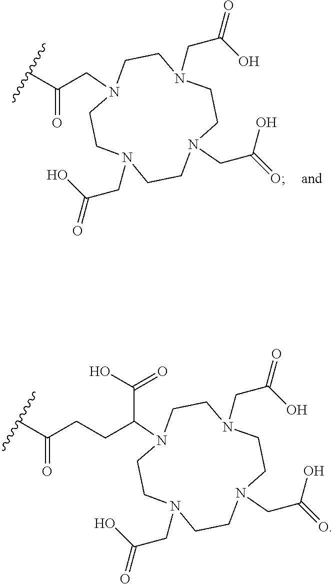 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

In an embodiment of a compound of Formula (I), or a pharmaceutically acceptable salt thereof, Ch¹ is:

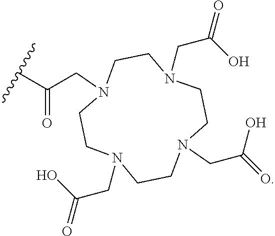 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof, M¹ is a radionuclide. In an embodiment of a compound of Formula (I), or a pharmaceutically acceptable salt thereof, M¹ is ¹⁸⁸Re, ¹⁸⁶Re, ¹⁵³Sm, ⁶⁶Ho, ⁶⁸Ga, ⁶⁷Ga, ⁸⁹Zr, ⁹⁰Y, ⁸⁹Sr, ¹¹¹In, ¹⁵³Gd, ²²⁵Ac, ²¹²Bi, ²¹³Bi, ²¹¹At, ⁶⁰Cu, ⁶¹Cu, ⁶⁷Cu, ⁶⁴Cu, ⁶²Cu, ¹⁹⁸Au, ⁹⁹Au, ^{195m}Pt, ^{193m}Pt, ¹⁹⁷Pt, ^{117m}Sn, ¹⁰³Pd, ¹⁰⁵Rh, ^{103m}Rh, ¹⁷⁷Lu, ²²³Ra, ²²⁴Ra, ²²⁷Th, ²²⁹Th, ¹⁴⁹Tb, ¹⁶¹Tb, ³²P, ³³P, ¹²⁵I, ²⁰³Pb, ²¹²Pb, ²⁰¹Tl, ¹¹⁹Sb, ^{58m}Co, ⁴⁷Sc, ¹⁴⁹Pm, or ¹⁶¹Ho. In an embodiment of a compound of Formula (I), or a pharmaceutically acceptable salt thereof, M¹ is ¹⁷⁷Lu, ²¹²Pb, or ²²⁵Ac. In an embodiment of a compound of Formula (I), or a pharmaceutically acceptable salt thereof, M¹ is ¹⁷⁷Lu. In an embodiment of a compound of Formula (I), or a pharmaceutically acceptable salt thereof, M¹ is ²¹²Pb. In an embodiment of a compound of Formula (I), or a pharmaceutically acceptable salt thereof, M¹ is ²²⁵Ac.

In an embodiment of a compound of Formula (I), or a pharmaceutically acceptable salt thereof, M¹ is absent. In other embodiments, M¹ is a radionuclide complexed with Ch¹. Suitable radionuclides include, without limitation, ¹⁷⁷Lu, ²¹²Pb, and ²²⁵Ac; in certain embodiments M¹ is ¹⁷⁷Lu.

In an embodiment of a compound of Formula (I), or a pharmaceutically acceptable salt thereof, M¹ is a radionuclide suitable for diagnostic imaging (for example, a gamma-emitter or a positron-emitter). Illustrative diagnostic radionuclides include, but are not limited to, ⁶⁸Ga or ⁶⁷Ga (for PET or SPECT imaging, respectively), ⁶⁴Cu (for PET imaging, with a beta emission that can provide a therapeutic effect), ⁸⁹Zr (for PET imaging, particularly useful for longer imaging windows such as those employed in antibody-based imaging), and ¹¹¹In (for SPECT imaging and dosimetry). A compound of Formula (I) labeled with such an imaging radionuclide may be administered to a subject to visualize or detect cells that overexpress NTSR1, thereby enabling localization of NTSR1-positive tumors or metastases.

In an embodiment of a compound of Formula (I), or a pharmaceutically acceptable salt thereof, the compound is utilized in a theranostic approach. For instance, a compound of Formula (I) can be radiolabeled with a diagnostic radionuclide such as ⁶⁸Ga or ¹¹¹In to assess tumor uptake and localization via imaging, and a corresponding compound, optionally the same compound or a structurally analogous compound of Formula (I), may be radiolabeled with a therapeutic radionuclide, such as ²²⁵Ac or ¹⁷⁷Lu, for treatment. In an illustrative example, an ¹¹¹In-labeled compound of Formula (I) may be administered to a subject to image and confirm NTSR1-positive tumors via SPECT, followed by administration of a ²²⁵Ac-labeled compound of Formula (I) to provide targeted alpha-particle therapy to those tumors. Similarly, a ⁶⁸Ga-labeled version of a compound of Formula (I) may be used for PET imaging, for patient selection or monitoring of disease progression, and a corresponding ¹⁷⁷Lu- or ⁶⁷Cu-labeled version can be used to treat the cancer. These diagnostic/therapeutic radionuclide pairs, for example, but not limited to, ¹¹¹In/²²⁵Ac, ⁶⁸Ga/¹⁷⁷Lu, ⁶⁴Cu/⁶⁷Cu, exemplify a theranostic strategy that provides both imaging and therapy with compounds targeting the same molecular receptor.

In an embodiment of a compound of Formula (I), or a pharmaceutically acceptable salt thereof, Ch¹(M¹) is selected from:

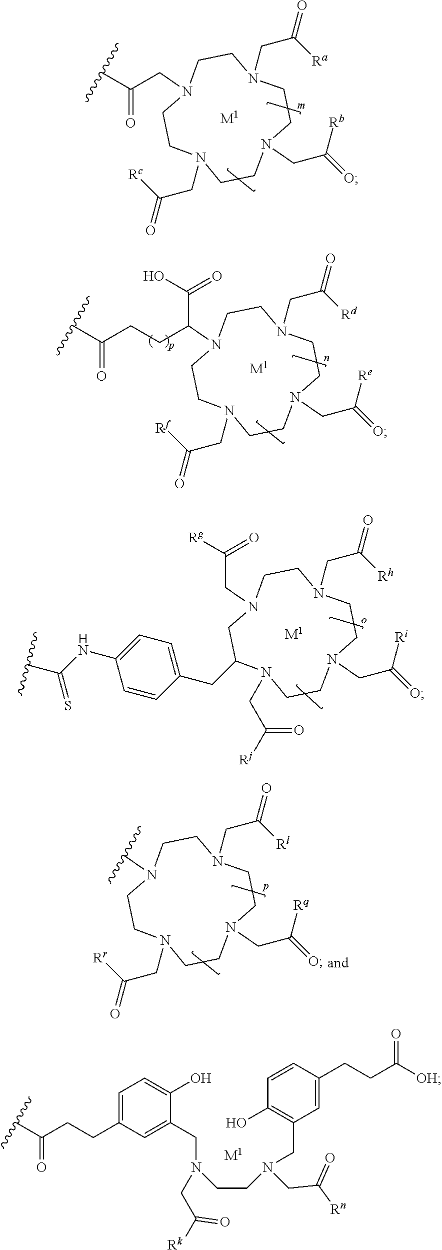

wherein R^{a}, R^{b}, R^{c}, R^{d}, R^{e}, R^{f}, R^{g}, R^{h}, Rⁱ, R^{j}, R^{k}, Rⁿ, R^{l}, R^{q}, and R^{r} are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac.

In an embodiment of a compound of Formula (I), or a pharmaceutically acceptable salt thereof, Ch¹(M¹) is selected from:

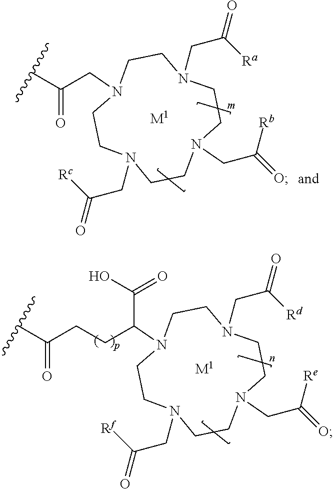

wherein R^{a}, R^{b}, R^{c}, R^{d}, R^{e}, and R^{f} are each independently selected from —OH and —NH₂; m, n, and p are each independently 0, 1, or 2; and M¹ is a radionuclide, preferably M¹ is ¹⁷⁷Lu, ²¹²Pb, or ²²⁵Ac.

In an embodiment of a compound of Formula (I), or a pharmaceutically acceptable salt thereof, Ch¹(M¹) is

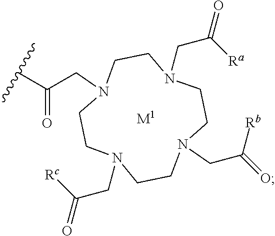

wherein R^{a}, R^{b}, and R^{c} are each independently selected from —OH and —NH₂; and M¹ is a radionuclide, preferably M¹ is ¹⁷⁷Lu, ²¹²Pb, or ²²⁵Ac. In an embodiment of a compound of Formula (I), or a pharmaceutically acceptable salt thereof, R^{a}, R^{b}, and R^{c} are each —OH. In an embodiment of a compound of Formula (I), or a pharmaceutically acceptable salt thereof, R^{a} and R^{c} are each —OH, and R^{b} is —NH₂. In an embodiment of a compound of Formula (I), or a pharmaceutically acceptable salt thereof, R^{a}, R^{b}, and R^{c} are each —NH₂.

In an embodiment of a compound of Formula (I), or a pharmaceutically acceptable salt thereof, Ch¹(M¹) is selected from:

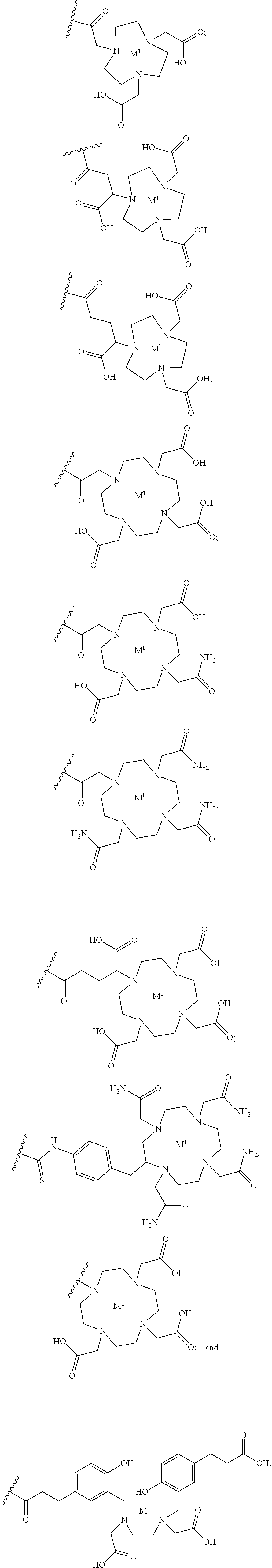

wherein M¹ is a radionuclide, preferably M¹ is ¹⁷⁷Lu, ²¹²Pb, or ²²⁵Ac.

In an embodiment of a compound of Formula (I), or a pharmaceutically acceptable salt thereof, Ch¹(M¹) is selected from:

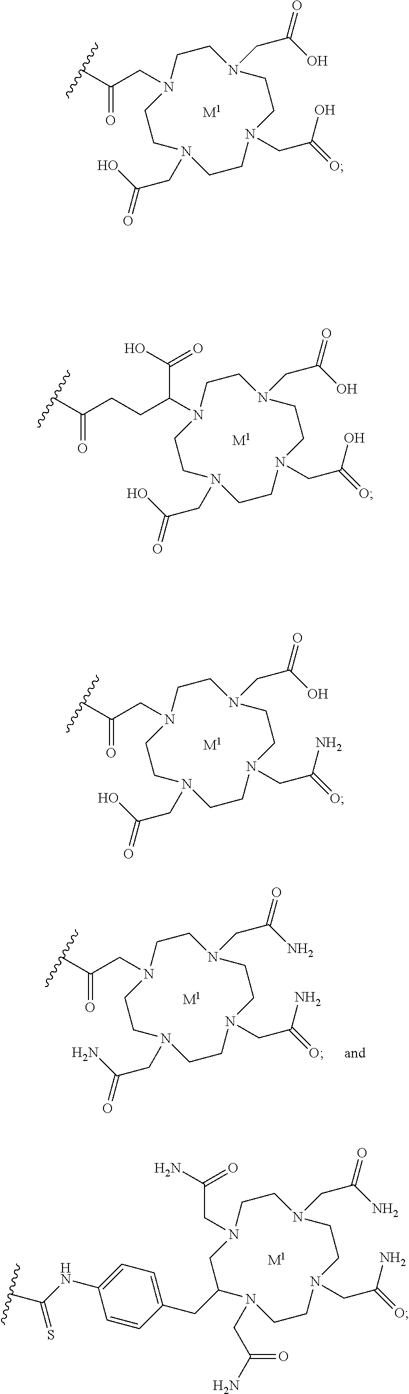

wherein M¹ is a radionuclide, preferably M¹ is ¹⁷⁷Lu, ²¹²Pb, or ²²⁵Ac.

In an embodiment of a compound of Formula (I), or a pharmaceutically acceptable salt thereof, Ch¹(M¹) is selected from:

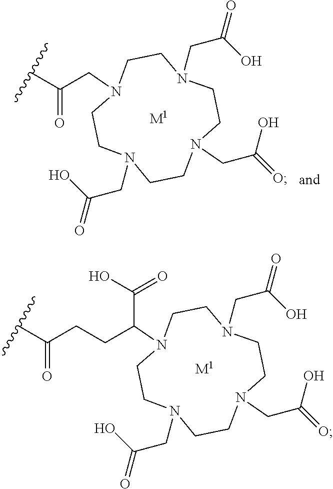

wherein M¹ is a radionuclide, preferably M¹ is ¹⁷⁷Lu, ²¹²Pb, or ²²⁵Ac.

In an embodiment of a compound of Formula (I), or a pharmaceutically acceptable salt thereof, Ch¹(M¹) is

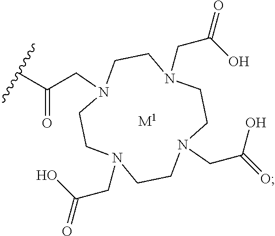

wherein M¹ is a radionuclide, preferably M¹ is ¹⁷⁷Lu, ²¹²Pb, or ²²⁵Ac.

In an embodiment of a compound of Formula (I), or a pharmaceutically acceptable salt thereof, Ch¹(M¹) is selected from:

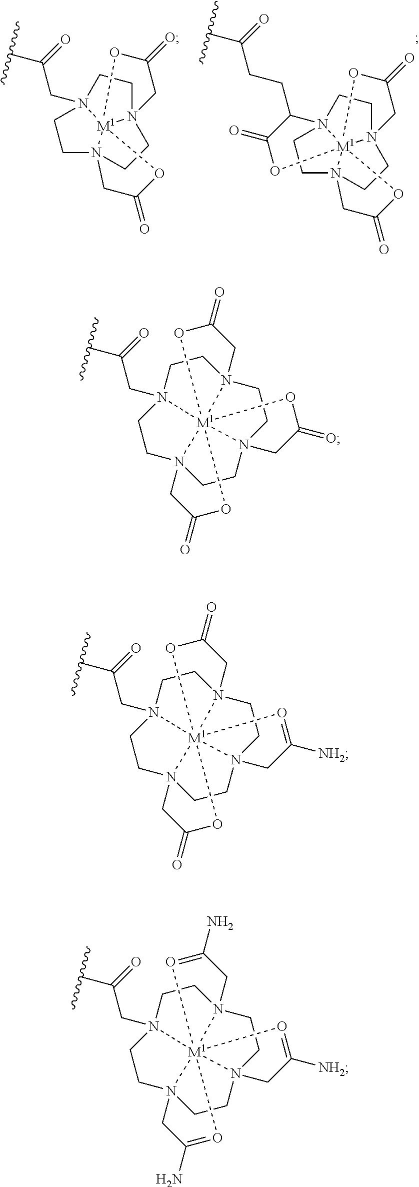

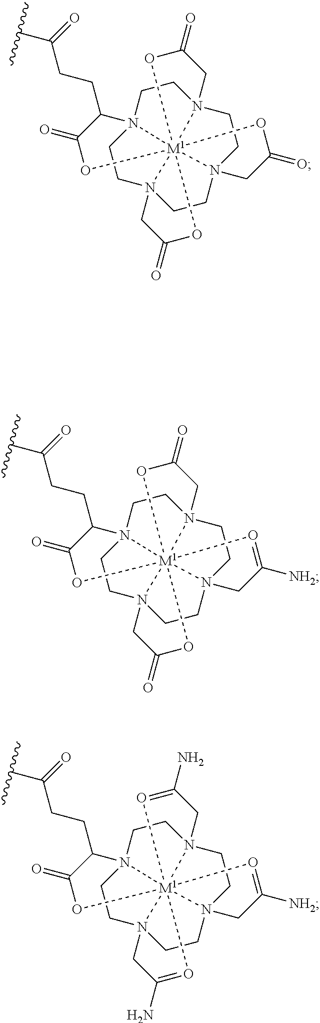

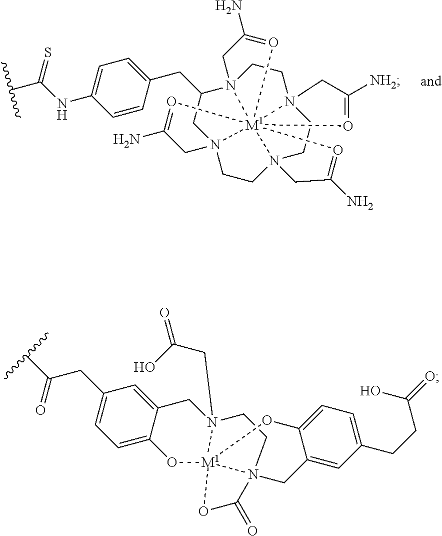

wherein M¹ is a radionuclide, preferably M¹ is ¹⁷⁷Lu, ²¹²Pb, or ²²⁵Ac.

In an embodiment of a compound of Formula (I), or a pharmaceutically acceptable salt thereof, Ch¹(M¹) is selected from:

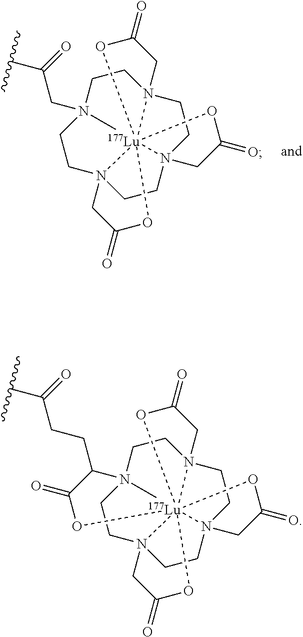

In an embodiment of a compound of Formula (I), or a pharmaceutically acceptable salt thereof, Ch¹(M¹) is

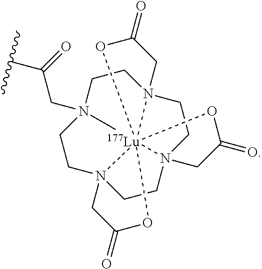

In an embodiment of a compound of Formula (I), or a pharmaceutically acceptable salt thereof, W is absent, Z is —COOH, R⁴ is a group of the formula

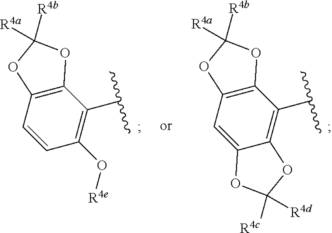

R^{4e} is methyl, and Cy¹ is phenyl or a 5- or 6-membered heteroaryl containing 1-3 heteroatoms independently selected from N, O, and S (provided that when Cy¹ is a bond, X¹ is a bond).

In an embodiment of a compound of Formula (I), or a pharmaceutically acceptable salt thereof, R¹ is isopropyl or tert-butyl, R² is adamantyl or adamantyl-CH₂—, and R³ is H. In an embodiment of a compound of Formula (I), or a pharmaceutically acceptable salt thereof, R¹ is isopropyl, R² is adamantyl or adamantyl-CH₂—, and R³ is H. In an embodiment of a compound of Formula (I), or a pharmaceutically acceptable salt thereof, R¹ is tert-butyl, R² is adamantyl or adamantyl-CH₂—, and R³ is H.

In an embodiment of a compound of Formula (I), or a pharmaceutically acceptable salt thereof, R¹ is isopropyl or tert-butyl, and R² and R³ together with the carbon atom to which they are attached form an adamantyl. In an embodiment of a compound of Formula (I), or a pharmaceutically acceptable salt thereof, R¹ is isopropyl, and R² and R³ together with the carbon atom to which they are attached form an adamantyl. In an embodiment of a compound of Formula (I), or a pharmaceutically acceptable salt thereof, R¹ is tert-butyl, and R² and R³ together with the carbon atom to which they are attached form an adamantyl.

In an embodiment of a compound of Formula (I), or a pharmaceutically acceptable salt thereof, R² is adamantyl or adamantyl-CH₂—, R³ is H, and R⁴ is 5-methoxybenzo[d][1,3]dioxol-4-yl. In an embodiment of a compound of Formula (I), or a pharmaceutically acceptable salt thereof, R² and R³ together with the carbon atom to which they are attached form an adamantyl, and R⁴ is 5-methoxybenzo[d][1,3]dioxol-4-yl.

In an embodiment of a compound of Formula (I), or a pharmaceutically acceptable salt thereof, R¹ is isopropyl or tert-butyl, R² is adamantyl or adamantyl-CH₂—, R³ is H, and R⁴ is 5-methoxybenzo[d][1,3]dioxol-4-yl. In an embodiment of a compound of Formula (I), or a pharmaceutically acceptable salt thereof, R¹ is isopropyl, R² is adamantyl or adamantyl-CH₂—, R³ is H, and R⁴ is 5-methoxybenzo[d][1,3]dioxol-4-yl. In an embodiment of a compound of Formula (I), or a pharmaceutically acceptable salt thereof, R¹ is tert-butyl, R² is adamantyl or adamantyl-CH₂—, R³ is H, and R⁴ is 5-methoxybenzo[d][1,3]dioxol-4-yl.

In an embodiment of a compound of Formula (I), or a pharmaceutically acceptable salt thereof, R¹ is isopropyl or tert-butyl, R² and R³ together with the carbon atom to which they are attached form an adamantyl, and R⁴ is 5-methoxybenzo[d][1,3]dioxol-4-yl. In an embodiment of a compound of Formula (I), or a pharmaceutically acceptable salt thereof, R¹ is isopropyl, R² and R³ together with the carbon atom to which they are attached form an adamantyl, and R⁴ is 5-methoxybenzo[d][1,3]dioxol-4-yl. In an embodiment of a compound of Formula (I), or a pharmaceutically acceptable salt thereof, R¹ is tert-butyl, R² and R³ together with the carbon atom to which they are attached form an adamantyl, and R⁴ is 5-methoxybenzo[d][1,3]dioxol-4-yl.

In an embodiment of a compound of Formula (I), or a pharmaceutically acceptable salt thereof, X¹ is a bond, and Cy¹ is a bond. In an embodiment of a compound of Formula (I), or a pharmaceutically acceptable salt thereof, X¹ is a bond, and Cy¹ is a phenyl. In an embodiment of a compound of Formula (I), or a pharmaceutically acceptable salt thereof, X¹ is a bond, and Cy¹ is a 5- or 6-membered heteroaryl containing 1-3 heteroatoms independently selected from N, O, and S, preferably Cy¹ is triazolyl. In an embodiment of a compound of Formula (I), or a pharmaceutically acceptable salt thereof, X¹ is —O—, and Cy¹ is phenyl. In an embodiment of a compound of Formula (I), or a pharmaceutically acceptable salt thereof, X¹ is —NH—, and Cy¹ is phenyl.

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}, —O—, —NR^{x3}C(O)—, —C(O)NR^{x5}, —NR^{x7}C(O)NR^{x8}—, —NR^{x11}C(O)O—, —NR^{x13}C(O)S—, or —NR^{x19}S(O)₂NR^{x20}, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl; and
L¹ is

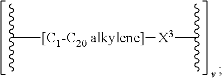

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₂₀ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene;
C₁-C₂₀ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl); and
v is 1, 2, 3, 4, or 5.

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x1}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}, —NR^{x7}C(O)NR^{x8}—, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl; and
L¹ is

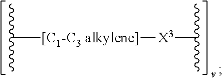

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₃ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene;
C₁-C₃ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl); and
v is 1, 2, 3, 4, or 5.

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}, —NR^{x7}C(O)NR^{x8}—, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl; and
L¹ is

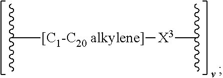

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5.

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}—, —NR^{x7}C(O)NR^{x8}—, —NR^{x11}C(O)O—, —NR^{x13}C(O)S—, or —NR^{x19}S(O)₂NR^{x20}, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl; and
L¹ is

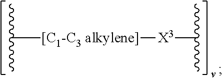

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5.

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}, preferably X² is a bond, —NR^{x2}—, —O—, —NR^{x3}C(O)—, —C(O)NR^{x5}—, —NR^{x7}C(O)NR^{x8}—, —NR^{x11}C(O)O—, —NR^{x13}C(O)S—, or —NR^{x19}S(O)₂NR^{x20}, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl; and
L¹ is

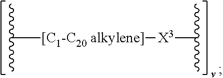

wherein X³ at each occurrence is independently selected from —NH—, —NCH₃—, —O—, and —NHC(O)—, and v is 1, 2, 3, 4, or 5.

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}, —NR^{x7}C(O)NR^{x8}—, —NR^{x11}C(O)O—, —NR^{x13}C(O)S—, or —NR^{x19}S(O)₂NR^{x20}, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl; and
L¹ is

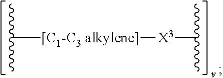

wherein X³ at each occurrence is independently selected from —NH—, —NCH₃—, —O—, and —NHC(O)—, and v is 1, 2, 3, 4, or 5.

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}—, —NR^{x7}C(O)NR^{x8}, —NR^{x11}C(O)O—, —NR^{x13}C(O)S—, or —NR^{x19}S(O)₂NR^{x20}, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl; and
L¹ is selected from

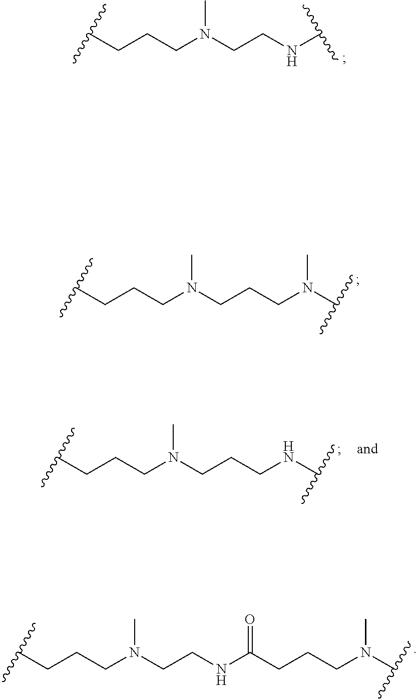

In an embodiment of a compound of Formula (I), or a pharmaceutically acceptable salt thereof, X²-L¹ is selected from

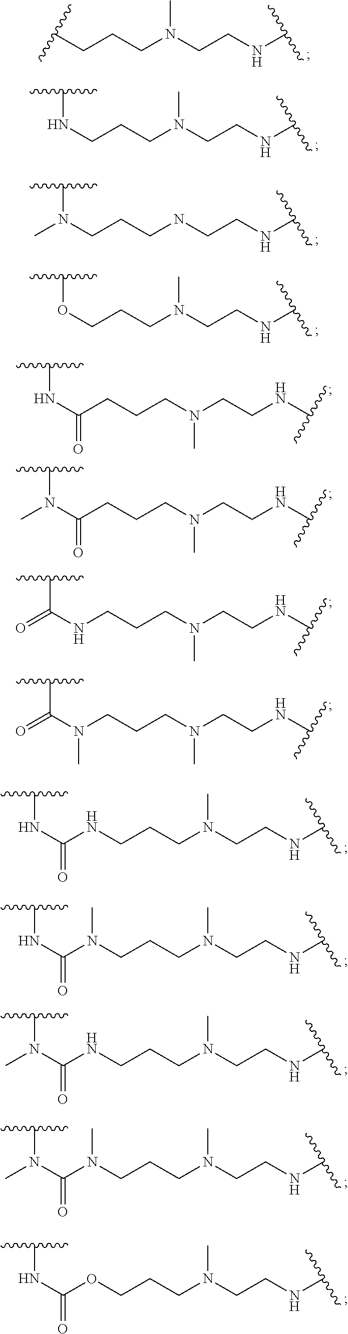

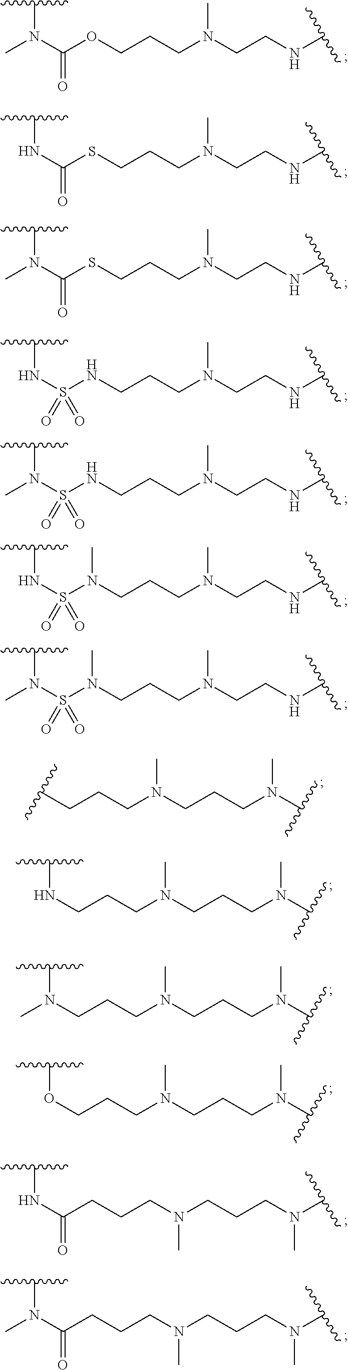

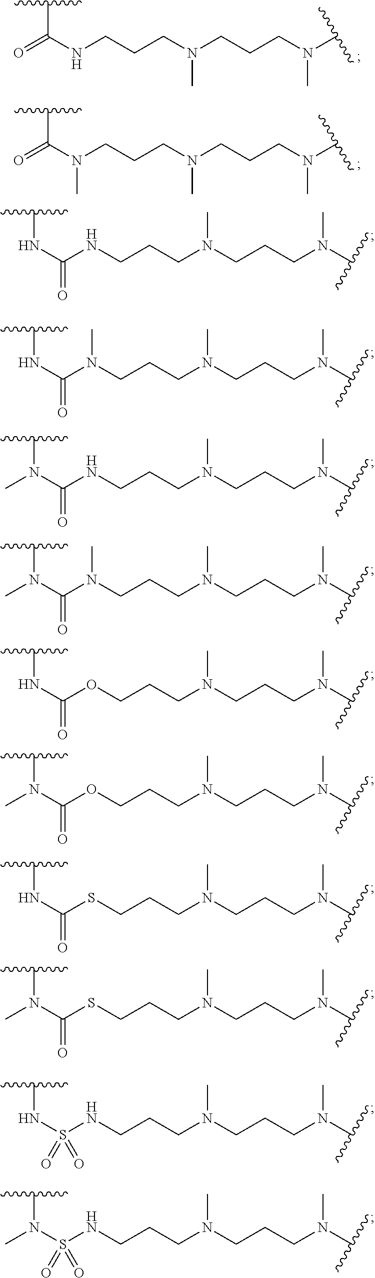

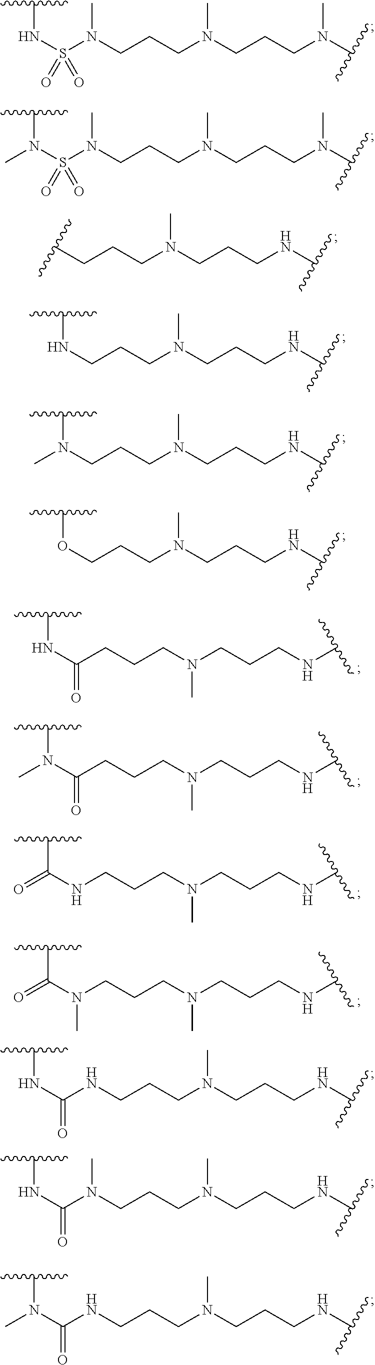

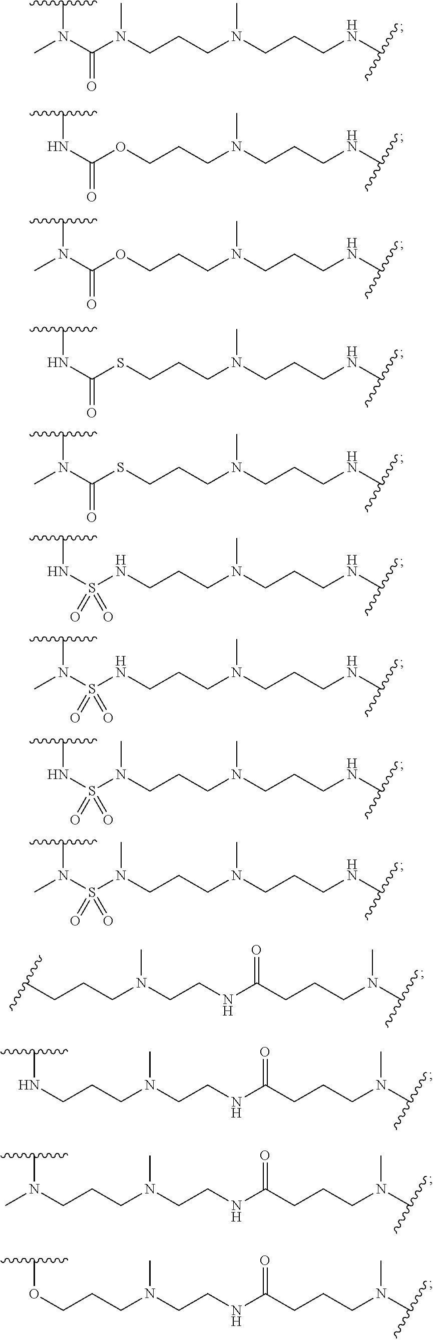

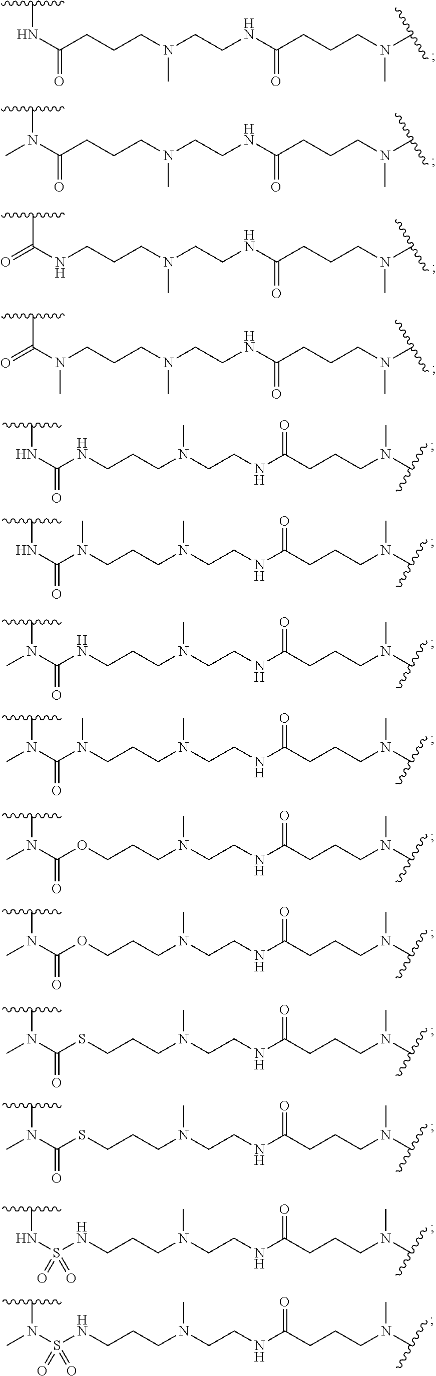

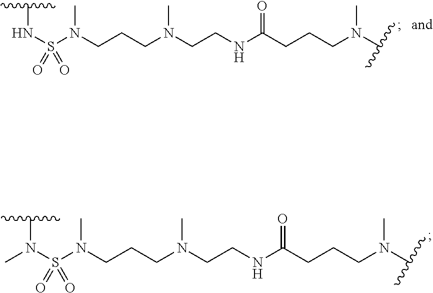 [SMILES: *NS(=O)(=O)N(C)CCCN(C)CCNC(=O)CCCN(*)C ; *N(C)CCCC(=O)NCCN(C)CCCN(C)S(=O)(=O)N(*)C]

In an embodiment of a compound of Formula (I), or a pharmaceutically acceptable salt thereof, X²-L¹ is selected from

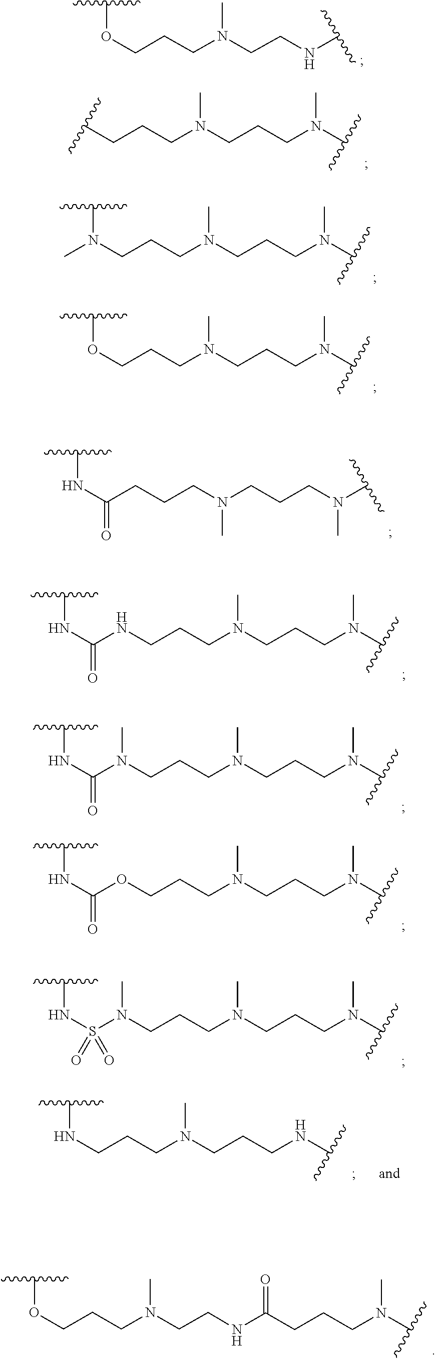

In an embodiment of a compound of Formula (I), or a pharmaceutically acceptable salt thereof,
L¹ is

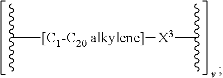

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₂₀ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene;
C₁-C₂₀ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl);
v is 1, 2, 3, 4, or 5; and
Ch¹ is selected from:

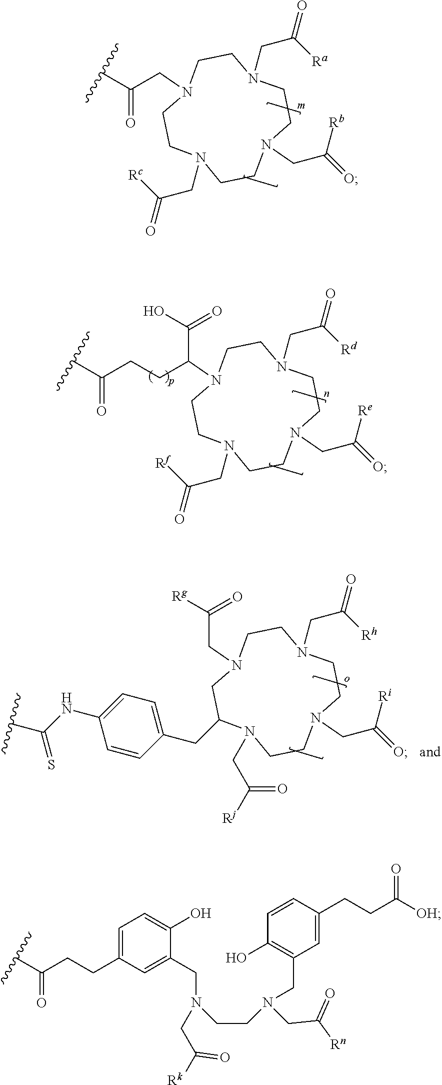

wherein R^{a}, R^{b}, R^{e}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

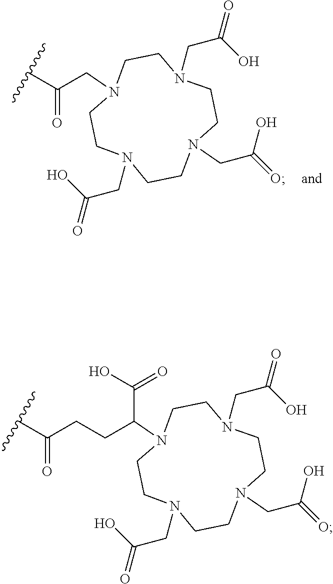 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is

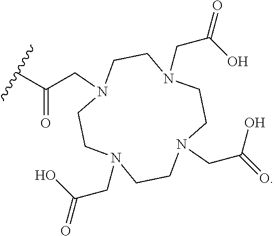 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
L¹ is

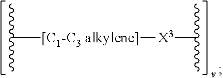

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₃ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene;
C₁-C₃ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl);
v is 1, 2, 3, 4, or 5; and
Ch¹ is selected from:

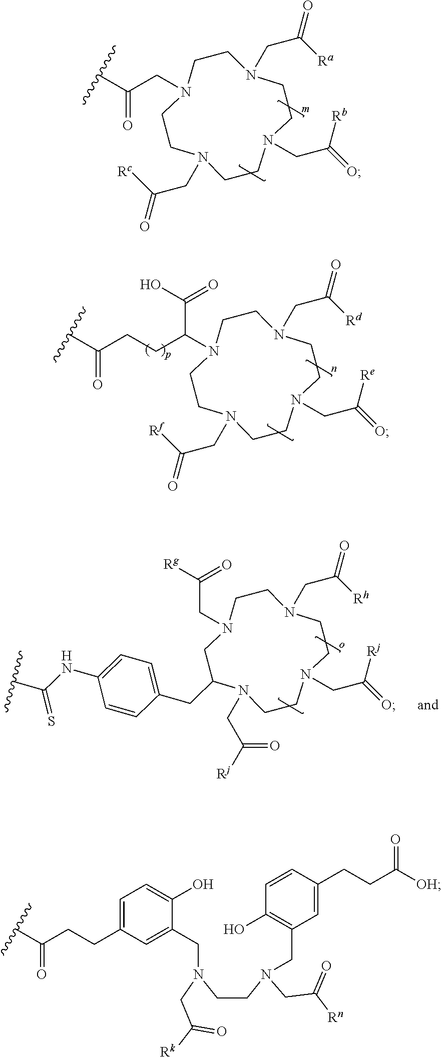

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

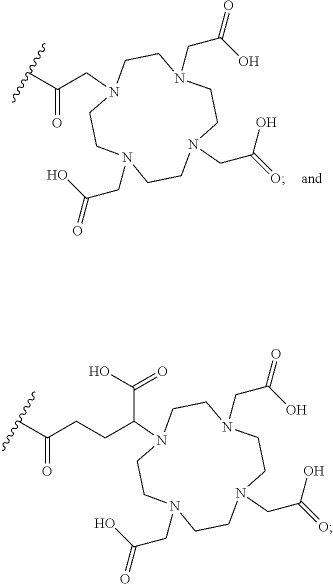 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is:

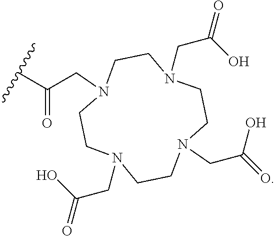 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
L¹ is

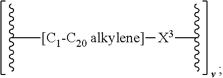

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5; and
Ch¹ is selected from:

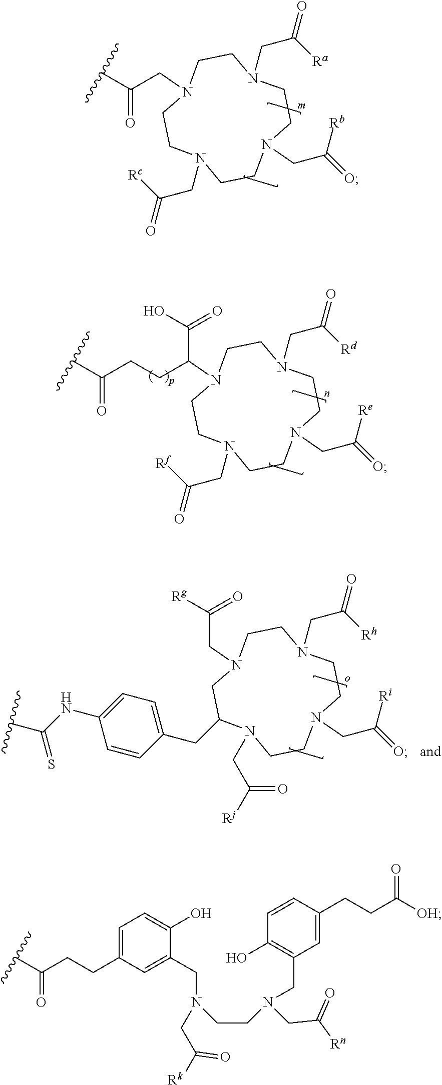

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

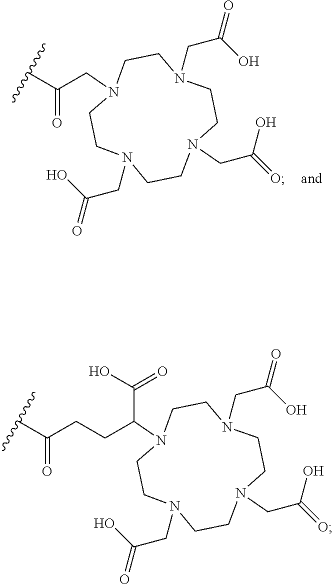 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is:

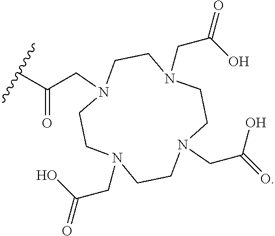 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
L¹ is

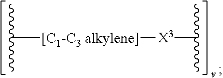

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5; and
Ch¹ is selected from:

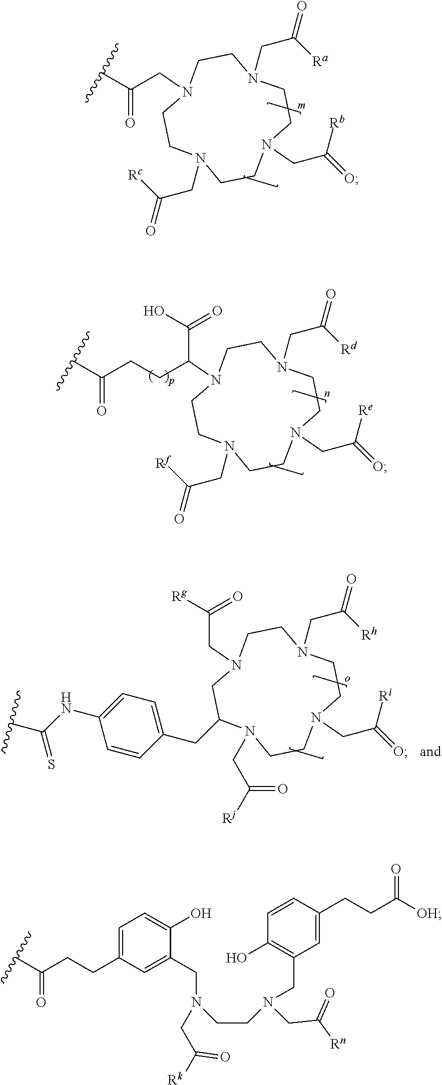

wherein R^{a}, R^{b}, R^{e}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

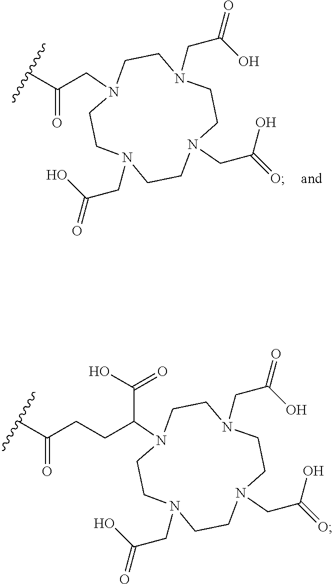 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is:

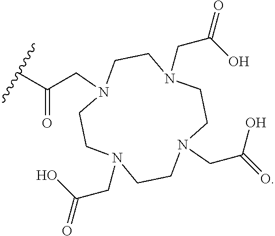 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
L¹ is

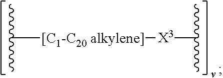

wherein X³ at each occurrence is independently selected from —NH—, —NCH₃—, —O—, and —NHC(O)—, and v is 1, 2, 3, 4, or 5; and
Ch¹ is selected from:

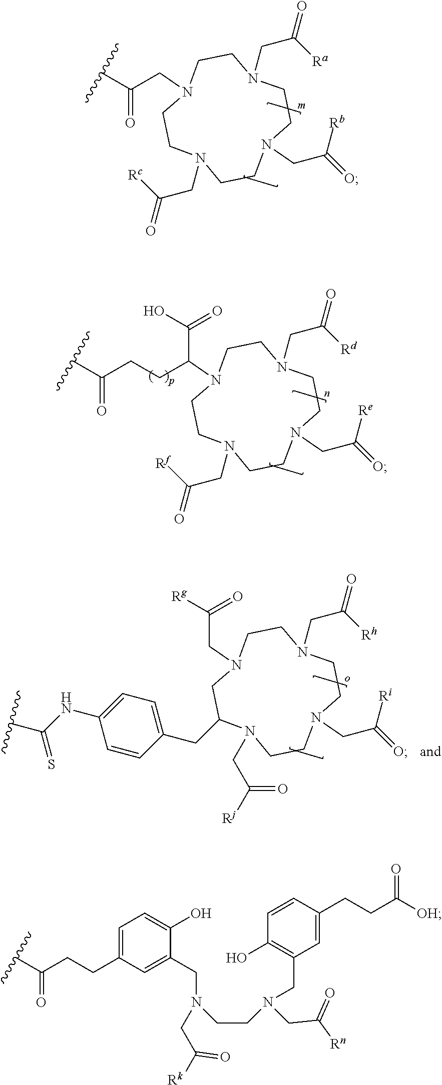

wherein R^{a}, R^{b}, R^{e}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

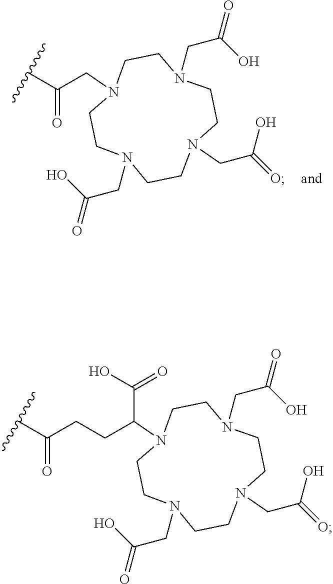 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is:

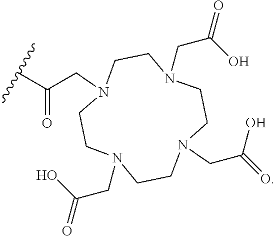 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
L¹ is

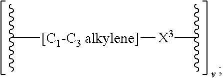

wherein X³ at each occurrence is independently selected from —NH—, —NCH₃—, —O—, and —NHC(O)—, and v is 1, 2, 3, 4, or 5; and
Ch¹ is selected from:

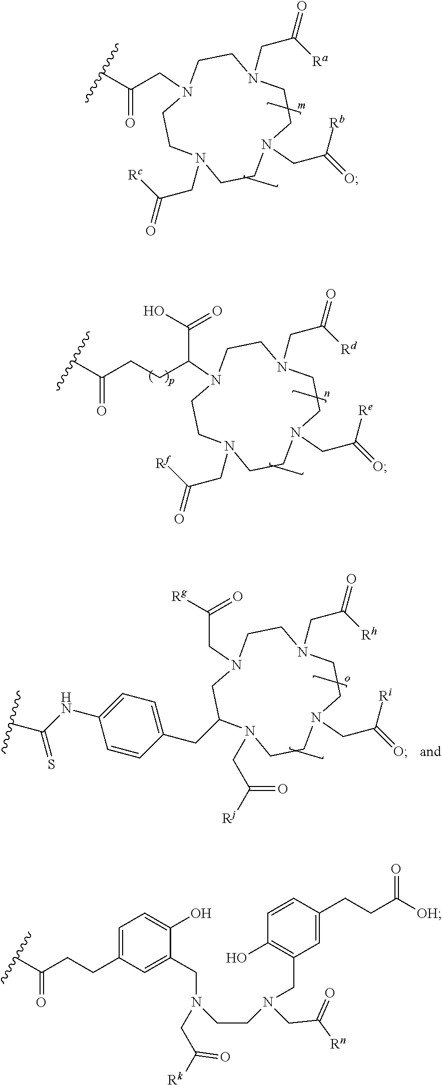

wherein R^{a}, R^{b}, R^{e}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

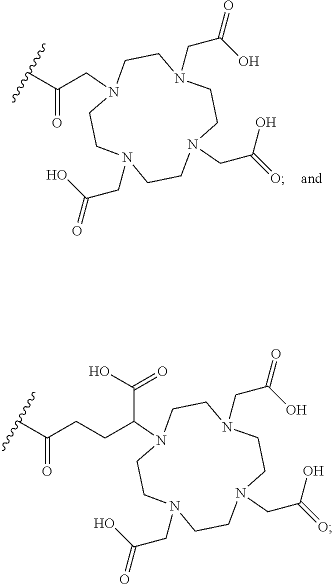 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is

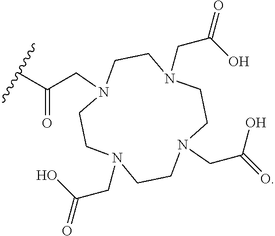 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
L¹ is

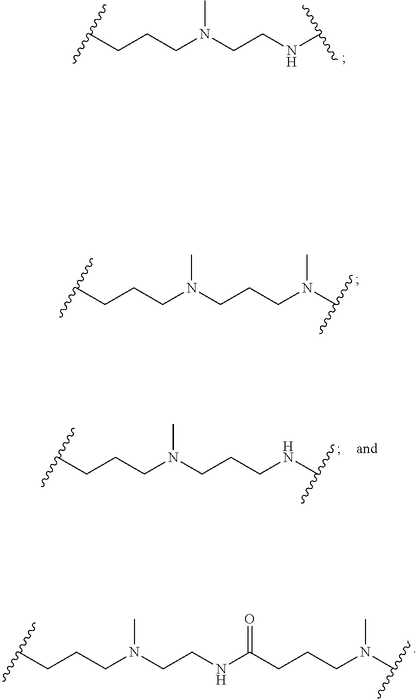

and Ch¹ is

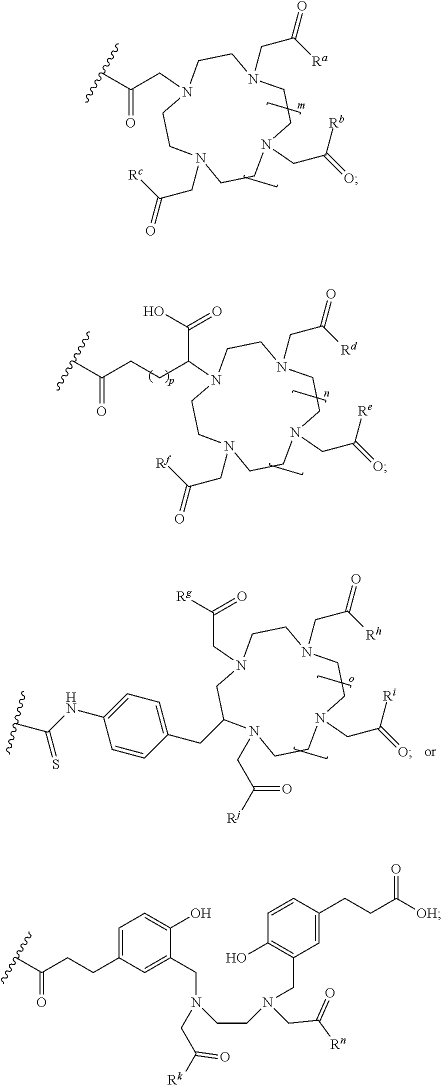

wherein R^{a}, R^{b}, R^{e}, R^{d}, R^{e}, R^{f}, R^{e}, R^{h}, Rⁱ, R^{j}, R^{k}, and Rⁿ are each independently selected from —OH and —NH₂; and m, n, o, and p are each independently 0, 1, or 2; preferably Ch¹ is

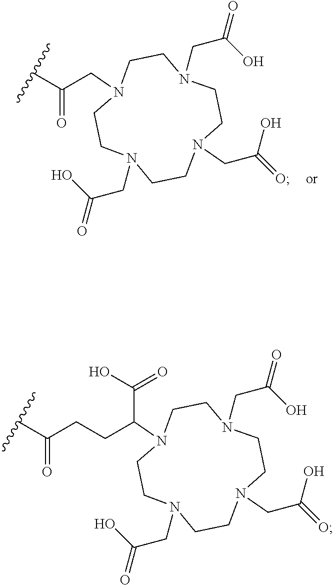 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is

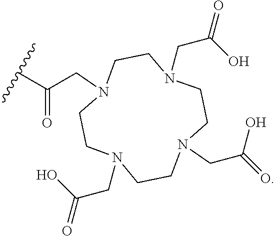 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof, L¹-Ch¹ is

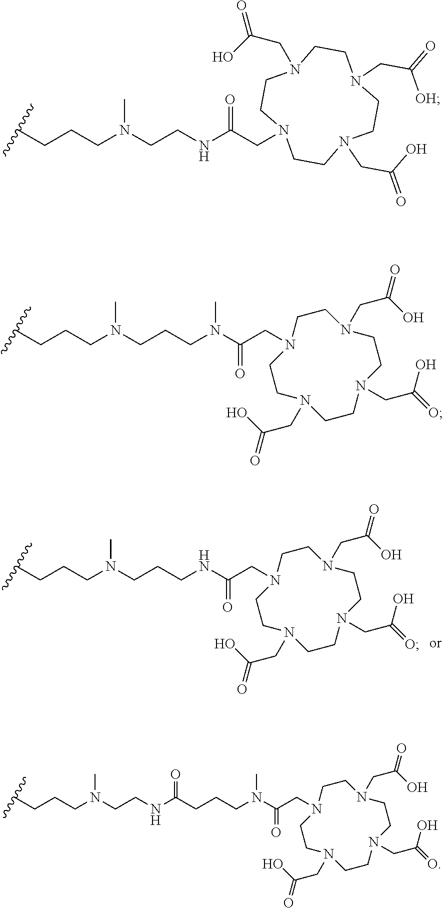 [SMILES: *CCCN(C)CCNC(=O)CN1CCN(CC(=O)O)CCN(CC(=O)O)CCN(CC(=O)O)CC1 ; *CCCN(C)CCCN(C)C(=O)CN1CCN(CC(=O)O)CCN(CC(=O)O)CCN(CC(=O)O)CC1 ; *CCCN(C)CCCNC(=O)CN1CCN(CC(=O)O)CCN(CC(=O)O)CCN(CC(=O)O)CC1 ; *CCCN(C)CCNC(=O)CCCN(C)C(=O)CN1CCN(CC(=O)O)CCN(CC(=O)O)CCN(CC(=O)O)CC1]

In an embodiment of a compound of Formula (I), or a pharmaceutically acceptable salt thereof,
L¹ is

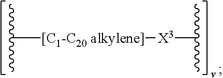

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}, —C(S)NR^{x25}, —NR^{x26}C(O)NR^{x27}, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₂₀ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene;
C₁-C₂₀ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl);
v is 1, 2, 3, 4, or 5; and
Ch¹(M¹) is

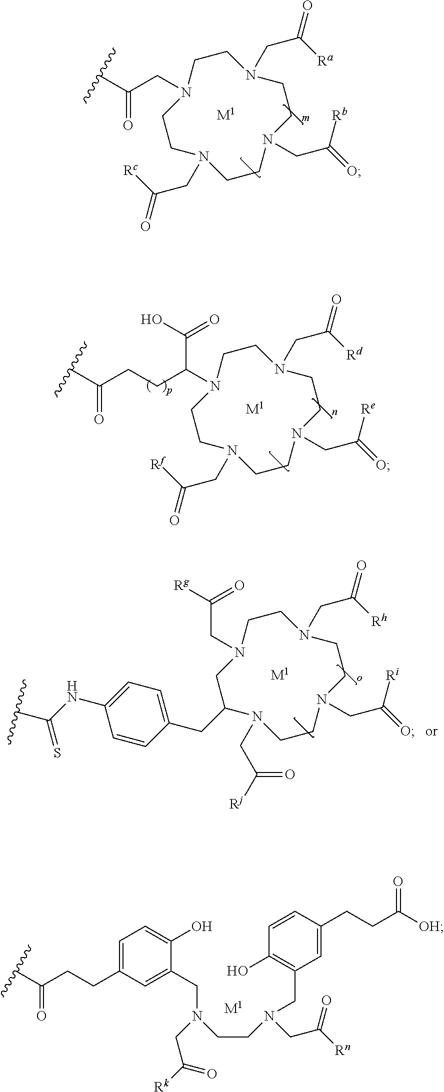

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

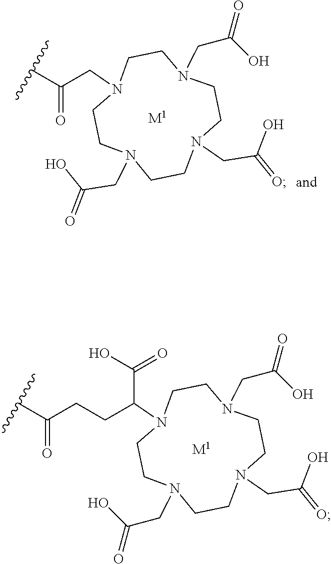

more preferably Ch¹(M¹) is:

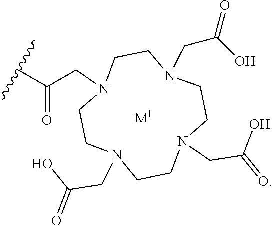

In an embodiment of a compound of Formula (I), or a pharmaceutically acceptable salt thereof,
L¹ is

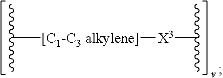

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₃ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene;
C₁-C₃ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl);
v is 1, 2, 3, 4, or 5; and
Ch¹(M¹) is selected from:

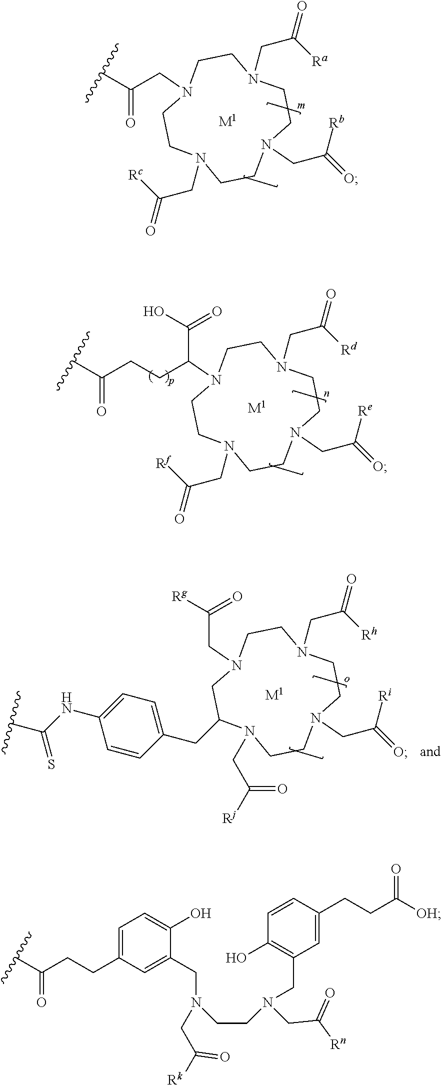

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

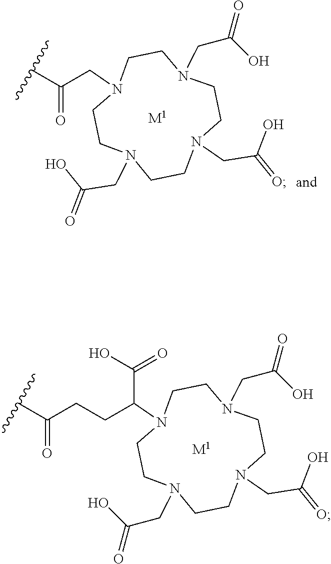

more preferably Ch¹(M¹) is:

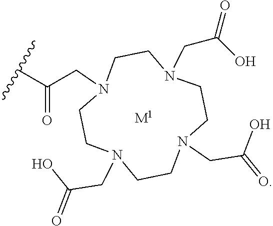

In an embodiment of a compound of Formula (I), or a pharmaceutically acceptable salt thereof,
L¹ is

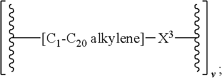

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5; and
Ch¹(M¹) is selected from:

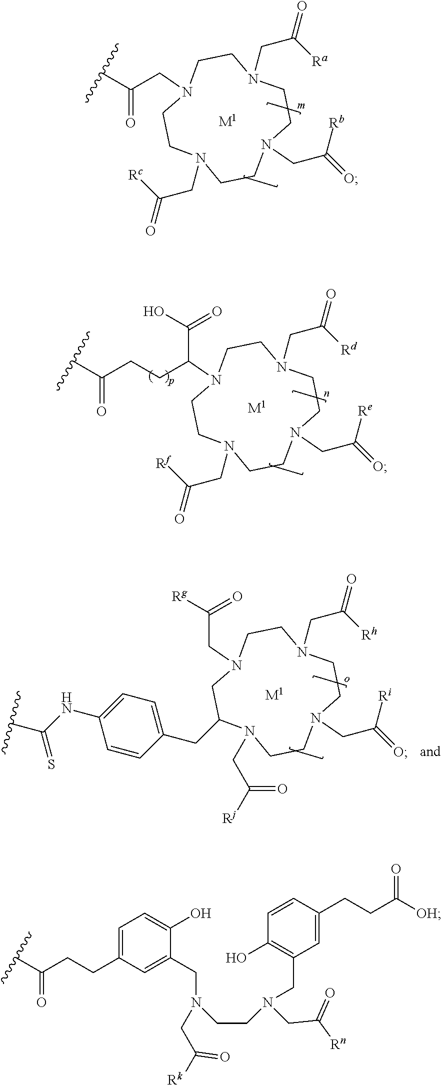

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

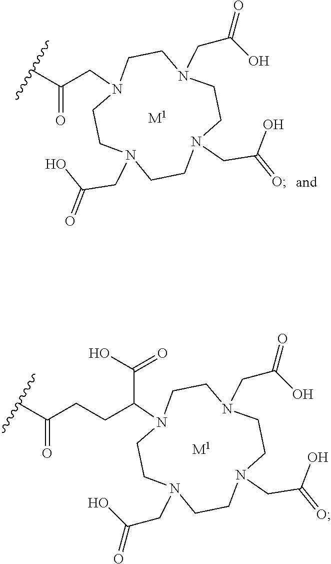

more preferably Ch¹(M¹) is:

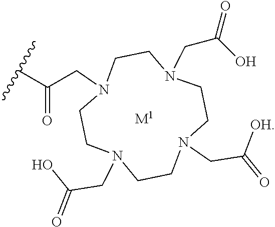

In an embodiment of a compound of Formula (I), or a pharmaceutically acceptable salt thereof,
L¹ is

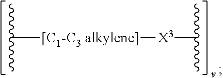

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5; and
Ch¹(M¹) is selected from:

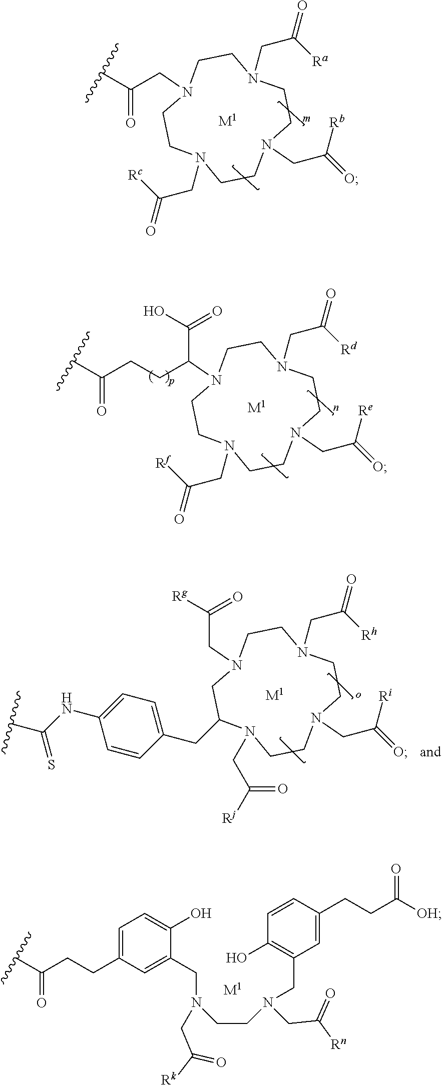

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

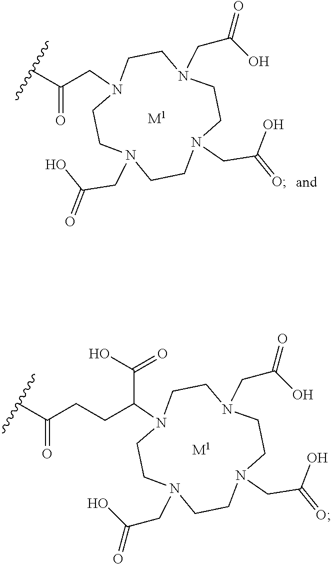

more preferably Ch¹(M¹) is:

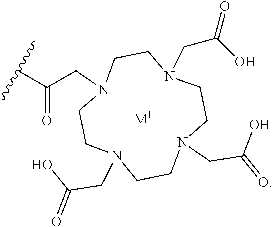

In an embodiment of a compound of Formula (I), or a pharmaceutically acceptable salt thereof,
L¹ is

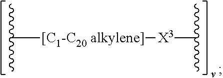

wherein X³ at each occurrence is independently selected from —NH—, —NCH₃—, —O—, and —NHC(O)—, and v is 1, 2, 3, 4, or 5; and
Ch¹(M¹) is selected from:

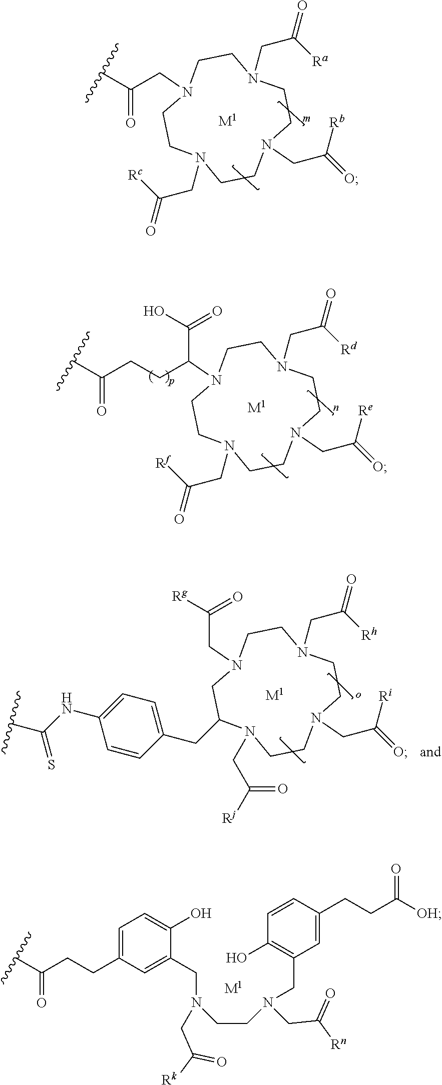

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

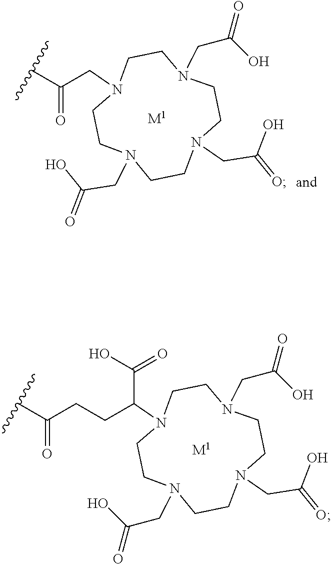

more preferably Ch¹(M¹) is:

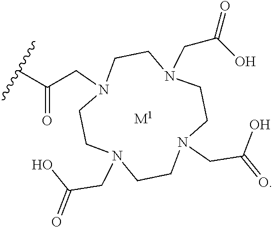

In an embodiment of a compound of Formula (I), or a pharmaceutically acceptable salt thereof,
L¹ is

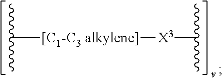

wherein X³ at each occurrence is independently selected from —NH—, —NCH₃—, —O—, and —NHC(O)—, and v is 1, 2, 3, 4, or 5; and
Ch¹(M¹) is selected from:

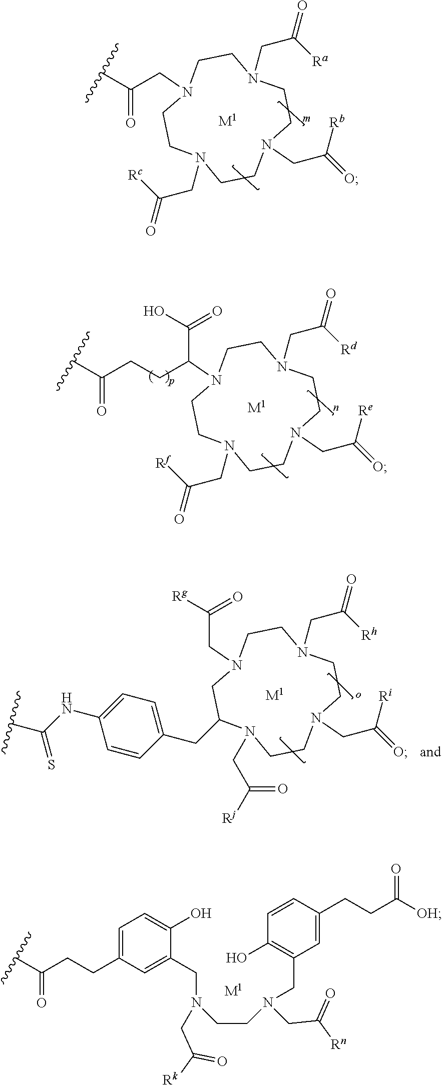

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

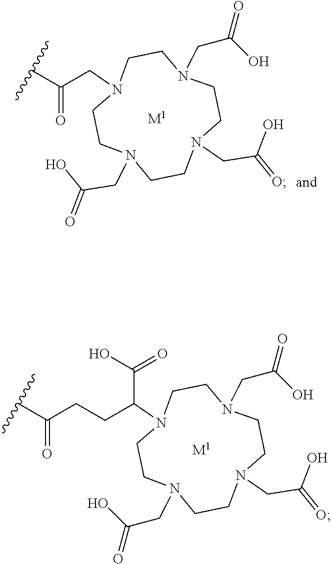

more preferably Ch¹(M¹) is:

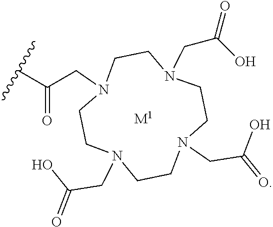

In an embodiment of a compound of Formula (I), or a pharmaceutically acceptable salt thereof,
L¹ is selected from

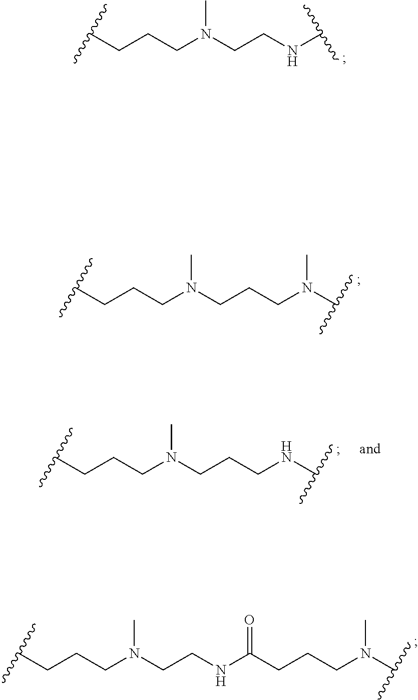

and Ch¹(M¹) is selected from:

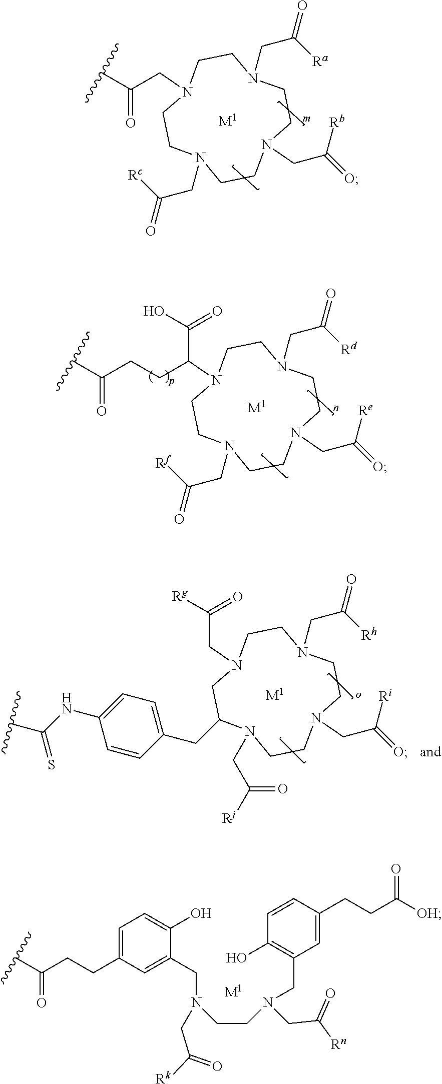

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

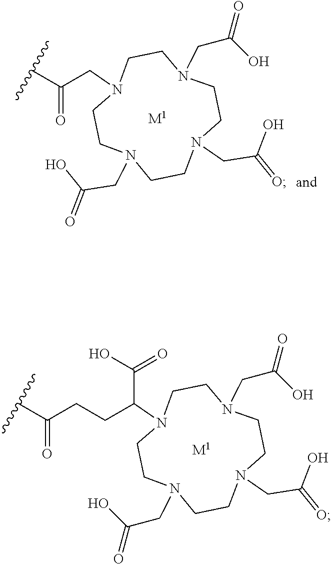

more preferably Ch¹(M¹) is:

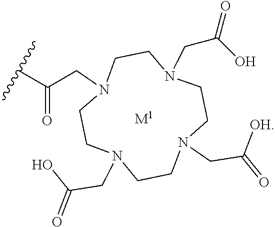

In an embodiment of a compound of Formula (I), or a pharmaceutically acceptable salt thereof, L¹-Ch¹(M¹) is selected from:

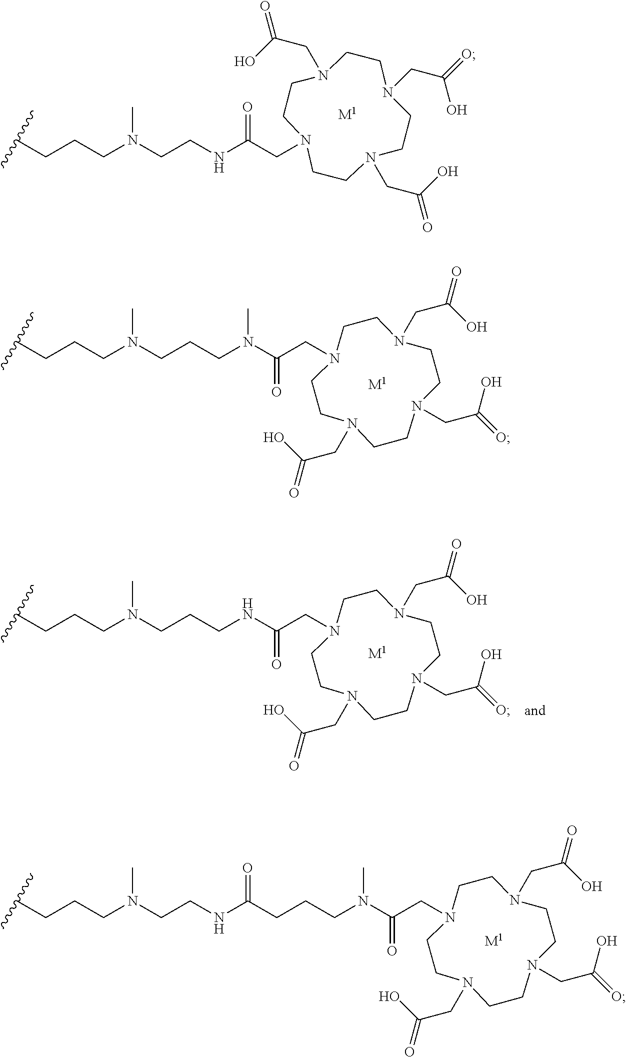

wherein M¹ is a radionuclide, preferably M¹ is ¹⁷⁷Lu, ²¹²Pb, or ²²⁵Ac.

In an embodiment of a compound of Formula (I), or a pharmaceutically acceptable salt thereof, L¹-Ch¹(M¹) is selected from:

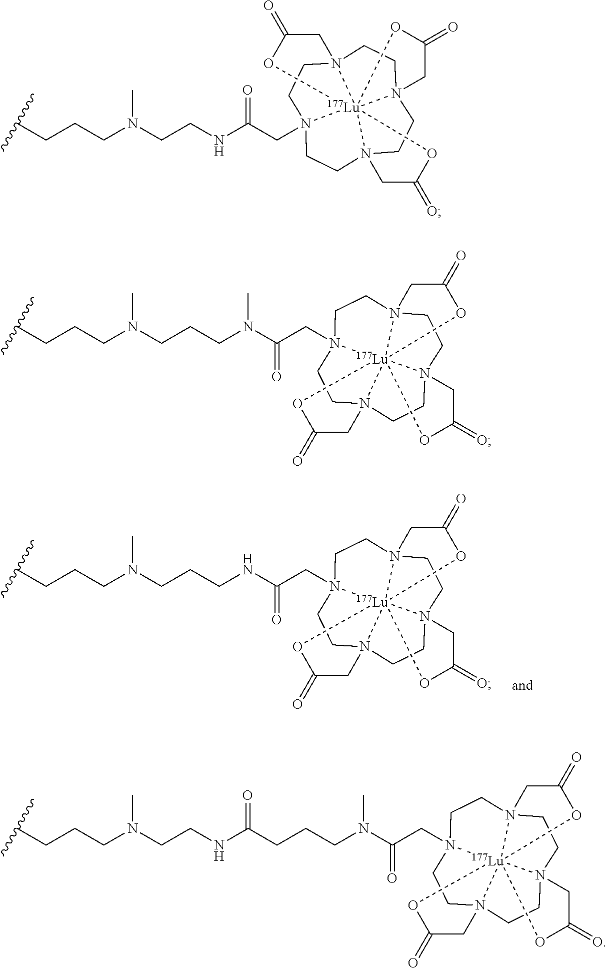

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}, —NR^{x7}C(O)NR^{x8}, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

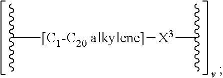

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₂₀ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene;
C₁-C₂₀ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl);
v is 1, 2, 3, 4, or 5; and
Ch¹ is selected from:

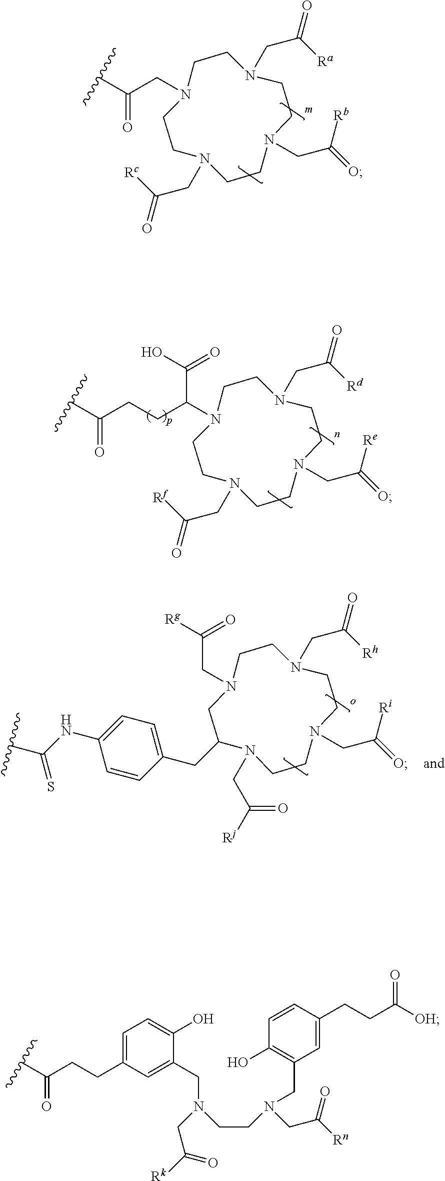

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

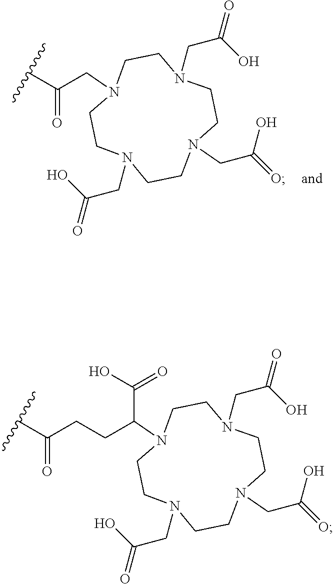 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is:

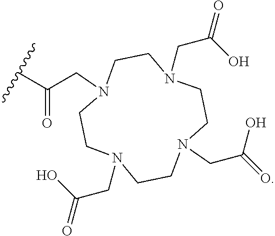 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}, —NR^{x7}C(O)NR^{x8}, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

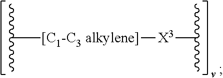

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₃ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene;
C₁-C₃ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl);
v is 1, 2, 3, 4, or 5; and
Ch¹ is selected from:

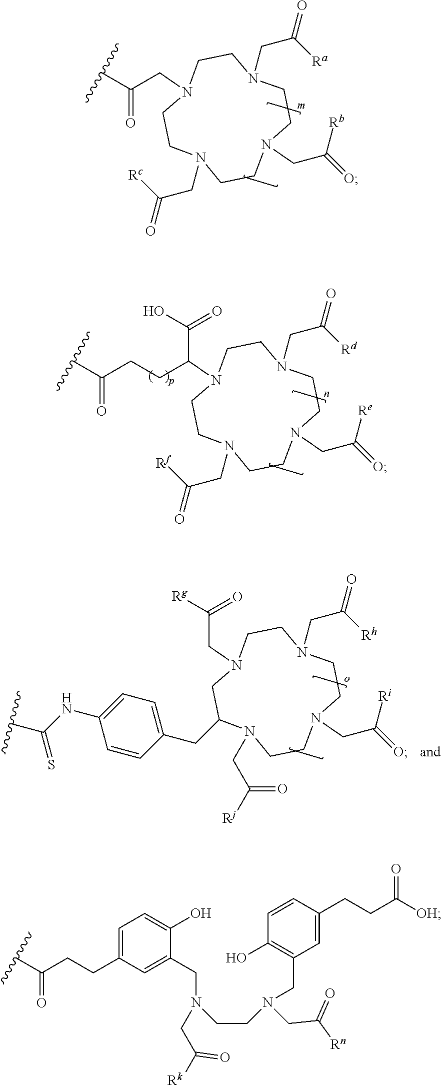

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

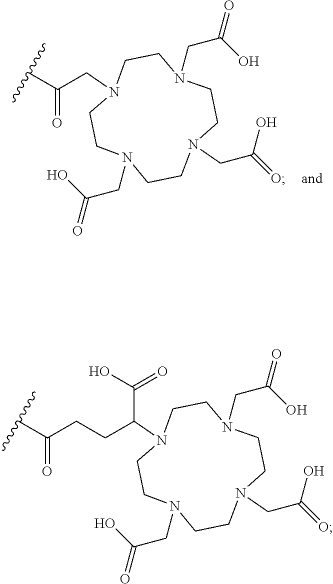 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is:

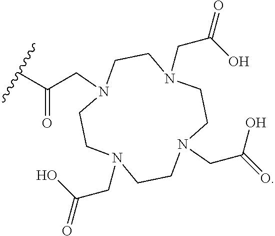 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NRC(O)NR^{x8}, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}—, —NR^{x7}C(O)NR^{x8}, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

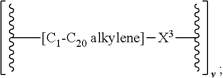

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5; and
Ch¹ is selected from:

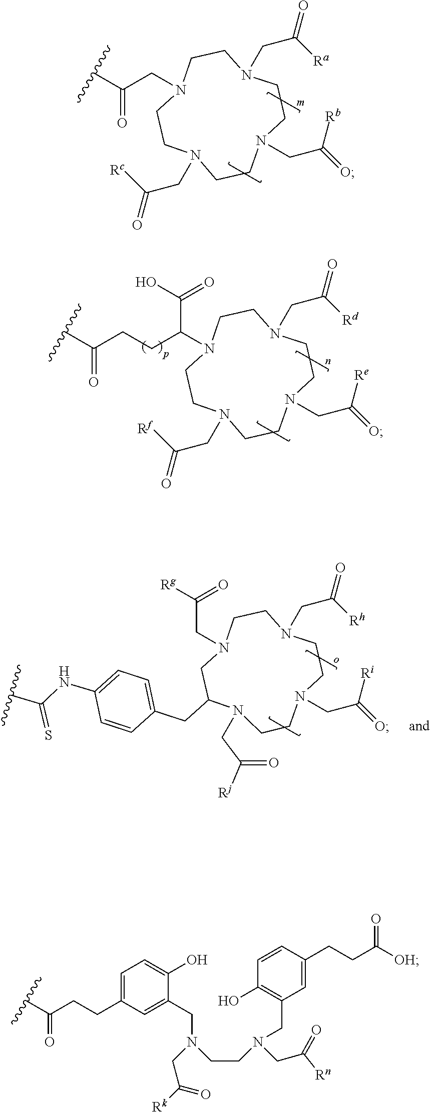

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

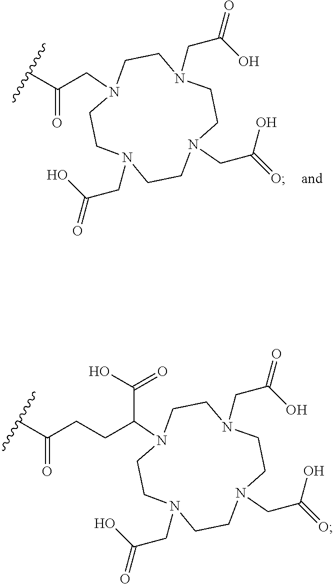 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is:

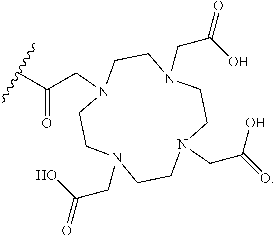 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}, —NR^{x1}C(O)NR^{x8}—, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

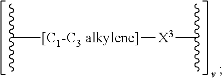

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5; and
Ch¹ is selected from:

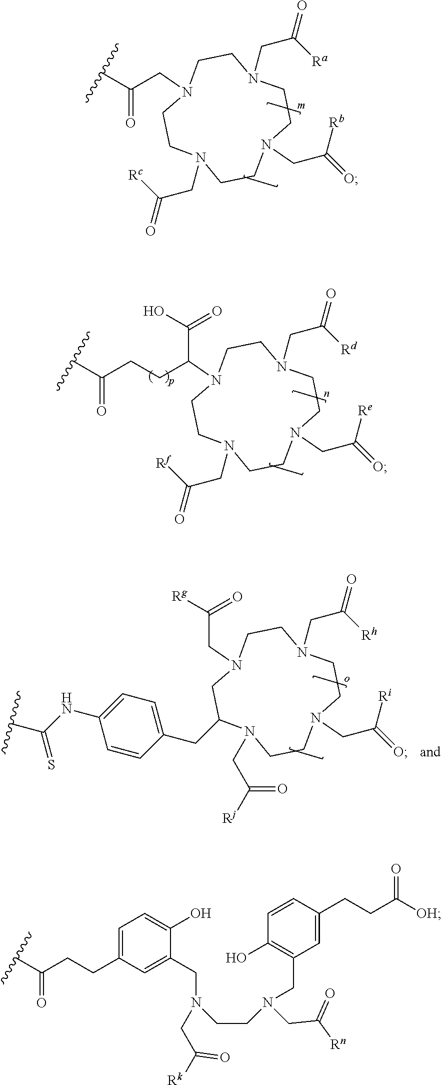

wherein R^{a}, R^{b}, R^{e}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

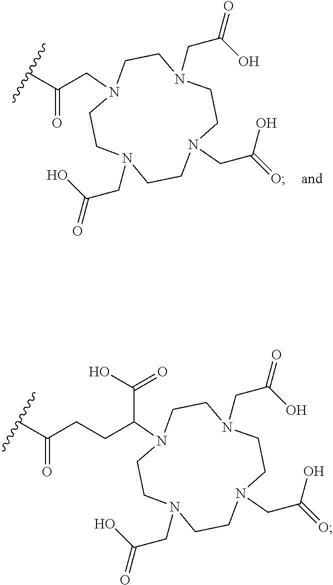 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is:

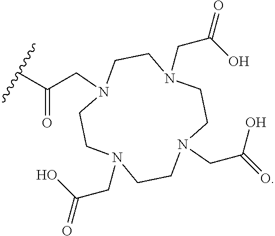 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}, —O—, —NR^{x3}C(O)—, —C(O)NR^{x5}, —NR^{x7}C(O)NR^{x8}—, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

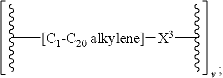

wherein X³ at each occurrence is independently selected from —NH—, —NCH₃—, —O—, and —NHC(O)—, and v is 1, 2, 3, 4, or 5; and
Ch¹ is selected from:

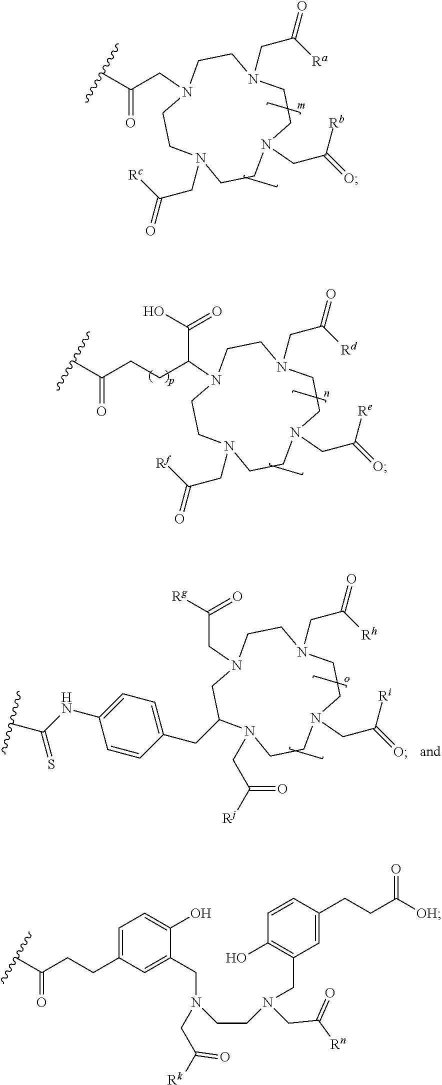

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

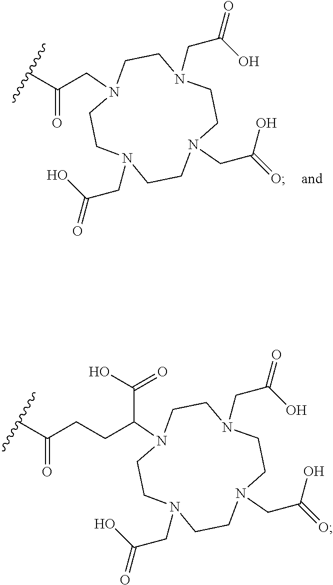 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is:

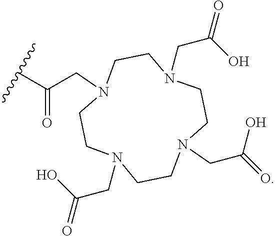 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}, —NR^{x1}C(O)NR^{x8}, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

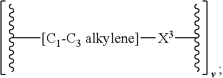

wherein X³ at each occurrence is independently selected from —NH—, —NCH₃—, —O—, and —NHC(O)—, and v is 1, 2, 3, 4, or 5; and
Ch¹ is selected from:

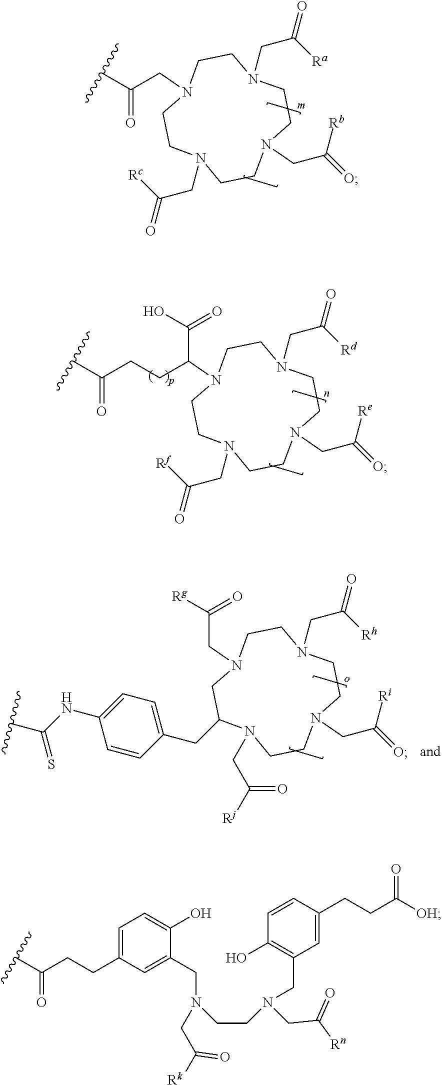

wherein R^{a}, R^{b}, R^{e}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

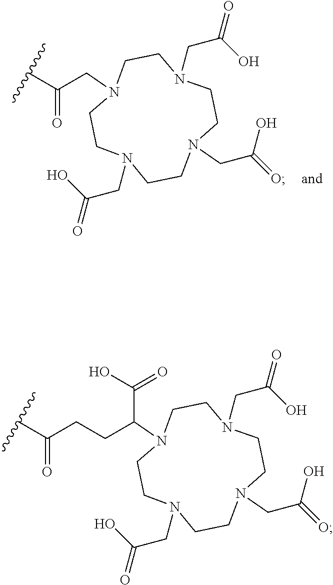 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is:

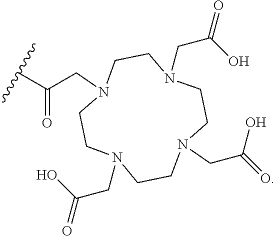 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof,
X² is a bond, —NR^{x2}, —O—, —S—, —C(O)—, —C(S)—, —S(O)—, —S(O)₂—, —NR^{x3}C(O)—, —NR^{x4}C(S)—, —C(O)NR^{x5}, —C(S)NR^{x6}—, —NR^{x7}C(O)NR^{x8}—, —NR^{x9}C(S)NR^{x10}, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}, —O—, —NR^{x3}C(O)—, —C(O)NR^{x5}, —NR^{x7}C(O)NR^{x8}—, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is selected from

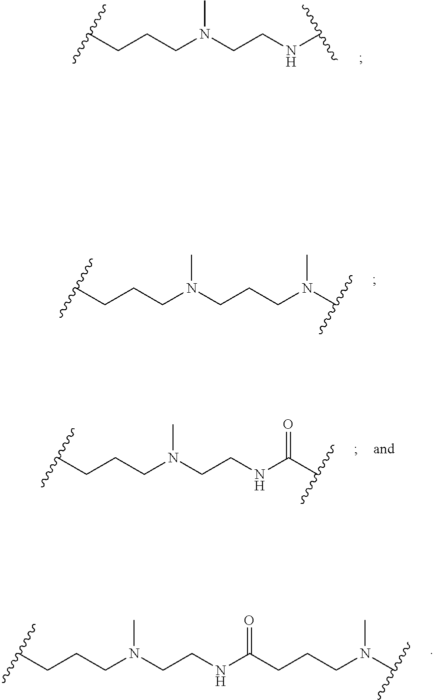

and Ch¹ is selected from:

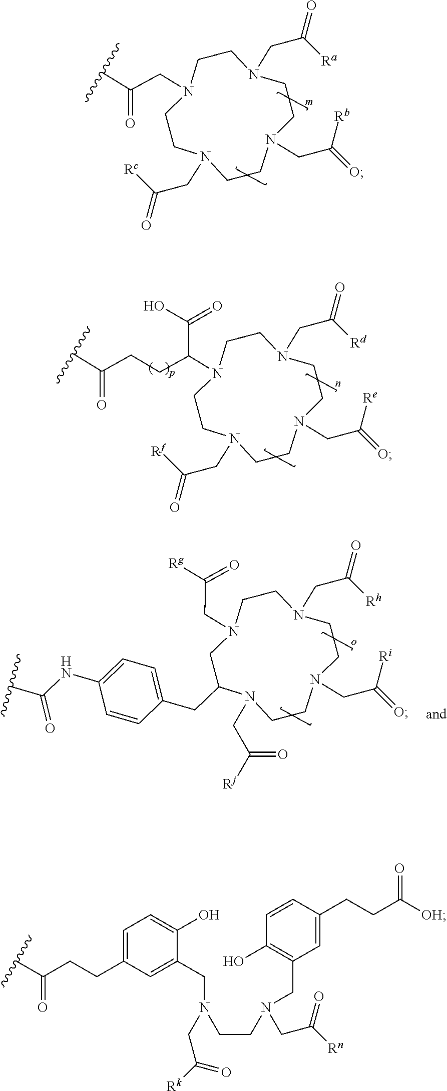

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2; preferably Ch¹ is selected from:

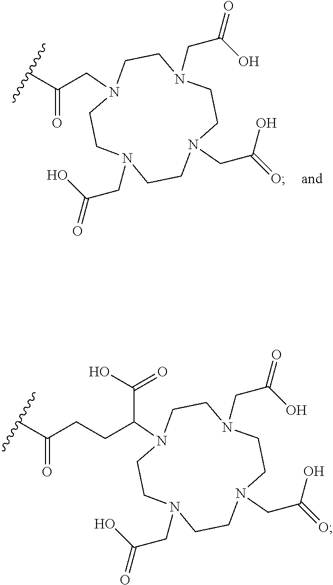 [SMILES: *C(=O)CN1CCN(CC(=O)O)CCN(CC(=O)O)CCN(CC(=O)O)CC1 ; *C(=O)CCC(C(=O)O)N1CCN(CC(=O)O)CCN(CC(=O)O)CCN(CC(=O)O)CC1]

more preferably Ch¹ is:

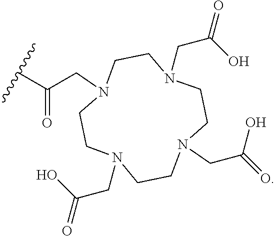 [SMILES: *C(=O)CN1CCN(CC(=O)O)CCN(CC(=O)O)CCN(CC(=O)O)CC1]

In an embodiment of a compound of Formula (I), or a pharmaceutically acceptable salt thereof, X²-L¹-Ch¹ is selected from:

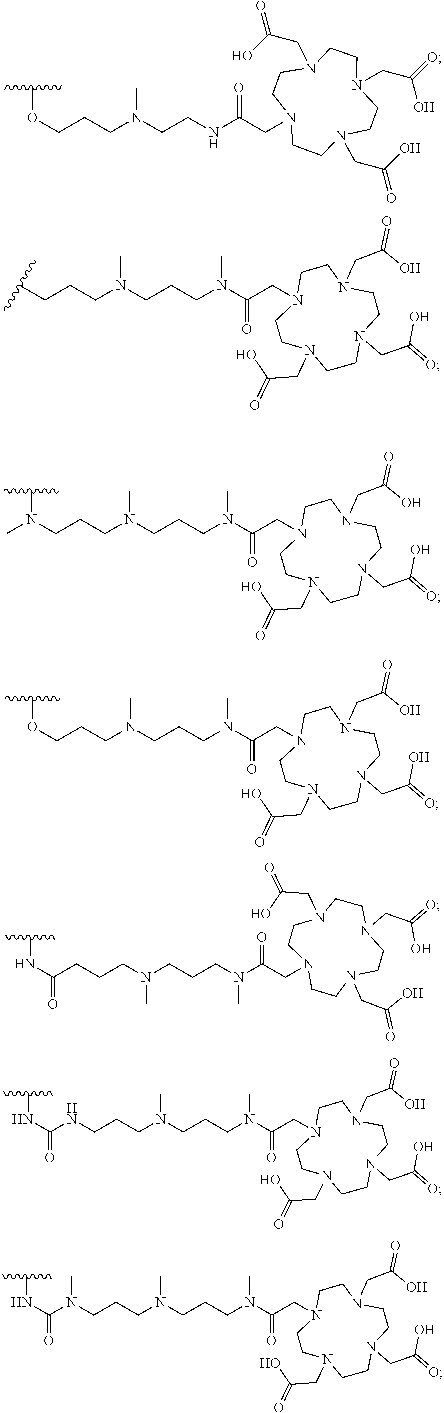

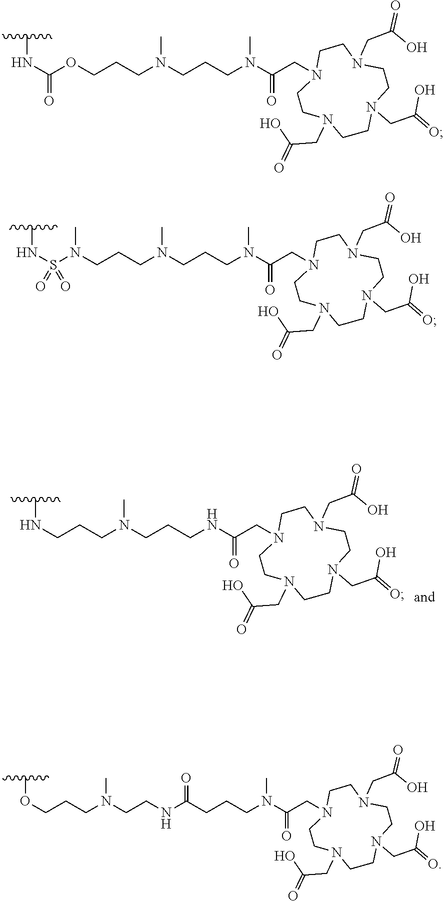 [SMILES: *NC(=O)OCCCN(C)CCCN(C)C(=O)CN1CCN(CC(=O)O)CCN(CC(=O)O)CCN(CC(=O)O)CC1 ; *NS(=O)(=O)N(C)CCCN(C)CCCN(C)C(=O)CN1CCN(CC(=O)O)CCN(CC(=O)O)CCN(CC(=O)O)CC1 ; *NCCCN(C)CCCNC(=O)CN1CCN(CC(=O)O)CCN(CC(=O)O)CCN(CC(=O)O)CC1 ; *OCCCN(C)CCNC(=O)CCCN(C)C(=O)CN1CCN(CC(=O)O)CCN(CC(=O)O)CCN(CC(=O)O)CC1]

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x1}C(O)NR^{x8}, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}—, —NR^{x7}C(O)NR^{x8}, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

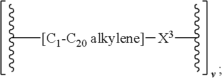

wherein
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₂₀ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene;
C₁-C₂₀ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl);
v is 1, 2, 3, 4, or 5; and
Ch¹(M¹) is selected from:

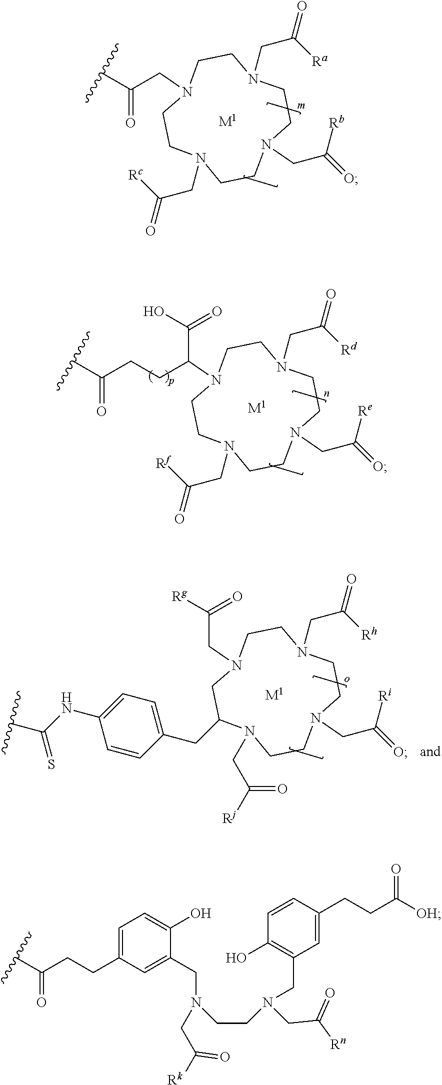

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

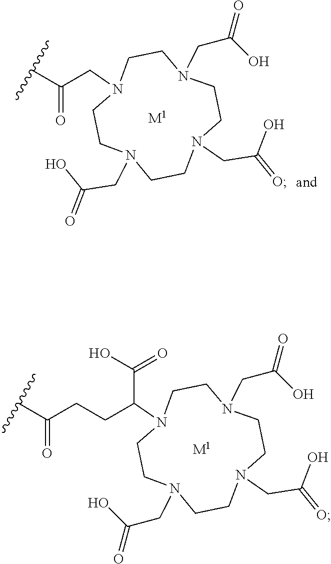

more preferably Ch¹(M¹) is:

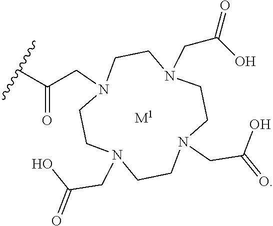

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}—, —NR^{x7}C(O)NR^{x8}, —NR^{x11}C(O)O—, —NR^{x13}C(O)S—, or —NR^{x19}S(O)₂NR^{x20}, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

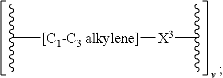

wherein
X³ at each occurrence is independently selected from —NR^{x21}—, —O—, —S—, —C(O)—, —C(S)—, —S(O)—, —S(O)₂—, —NR^{x2}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₃ alkylene at each occurrence is optionally interrupted by 1-5 groups independently selected from C₃-C₁₀ cycloalkylene, C₄-C₁₀ heterocycloalkylene, C₆-C₁₀ arylene, and C₅-C₁₀ heteroarylene;
C₁-C₃ alkylene, C₃-C₁₀ cycloalkylene, C₄-C₁₀ heterocycloalkylene, C₆-C₁₀ arylene, and C₅-C₁₀ heteroarylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl);
v is 1, 2, 3, 4, or 5; and
Ch¹(M¹) is selected from:

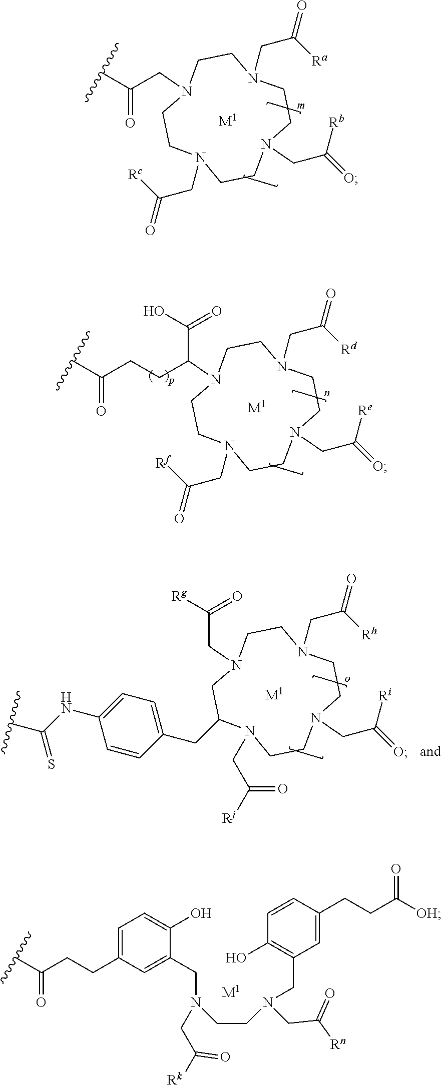

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

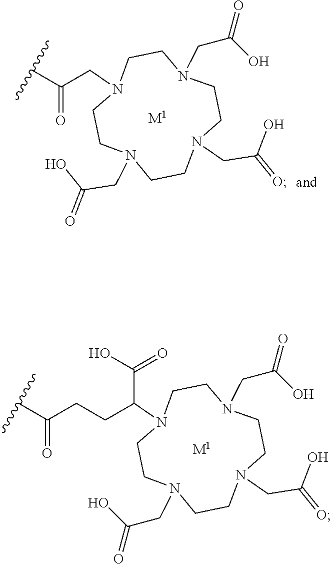

more preferably Ch¹(M¹) is:

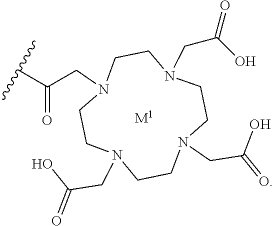

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}—, —NR^{x7}C(O)NR^{x8}—, —NR^{x11}C(O)O—, —NR^{x13}C(O)S—, or —NR^{x19}S(O)₂NR^{x20}, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

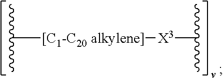

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5; and
Ch¹(M¹) is selected from:

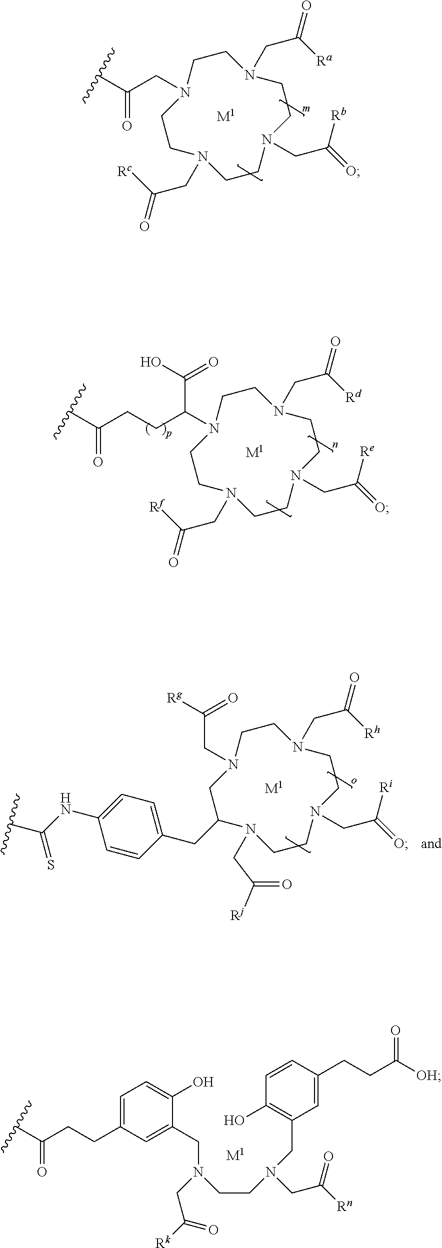

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂, m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

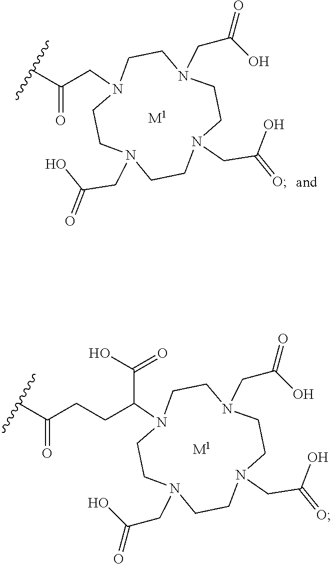

more preferably Ch¹(M¹) is:

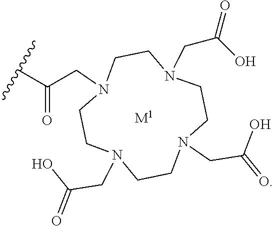

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}, —O—, —NR^{x3}C(O)—, —C(O)NR^{x5}—, —NR^{x7}C(O)NR^{x8}—, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

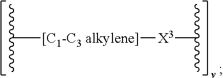

wherein X³ at each occurrence is independently selected from —NR^{x21}—, —O—, and —NR^{x22}C(O)—, R^{x21} and R^{x22} at each occurrence are independently selected from H and C₁-C₆ alkyl, and v is 1, 2, 3, 4, or 5; and
Ch¹(M¹) is selected from:

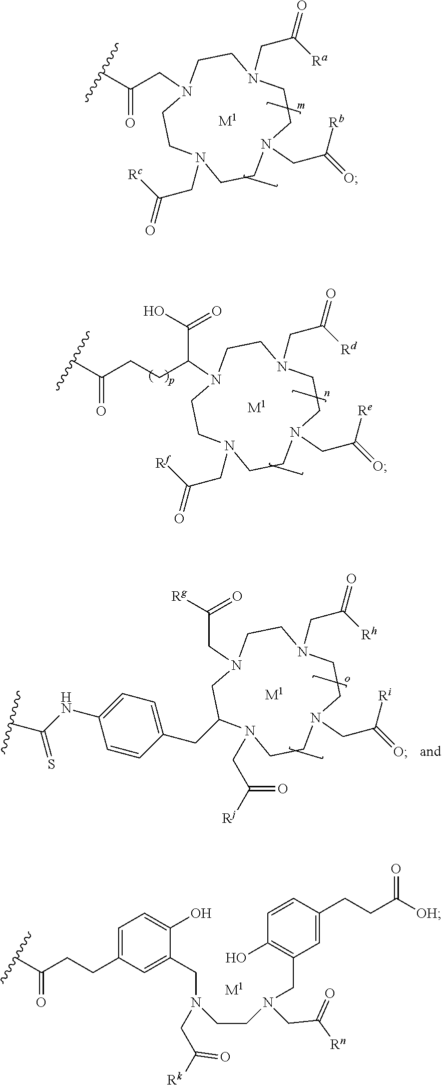

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

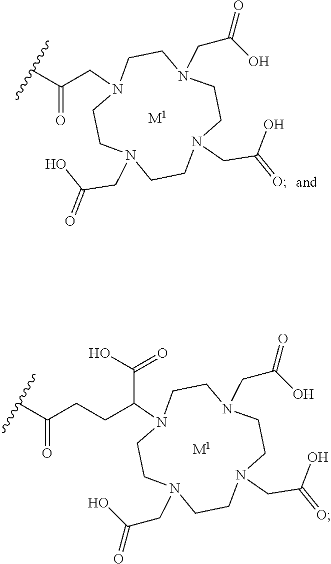

more preferably Ch¹(M¹) is:

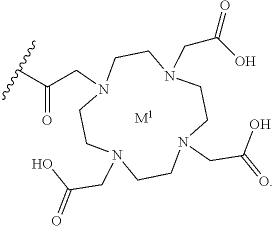

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}—, —NR^{x7}C(O)NR^{x8}—, —NR^{x11}C(O)O—, —NR^{x13}C(O)S—, or —NR^{x19}S(O)₂NR^{x20}, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

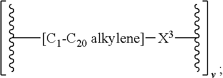

wherein X³ at each occurrence is independently selected from —NH—, —NCH₃—, —O—, and —NHC(O)—, and v is 1, 2, 3, 4, or 5; and
Ch¹(M¹) is selected from:

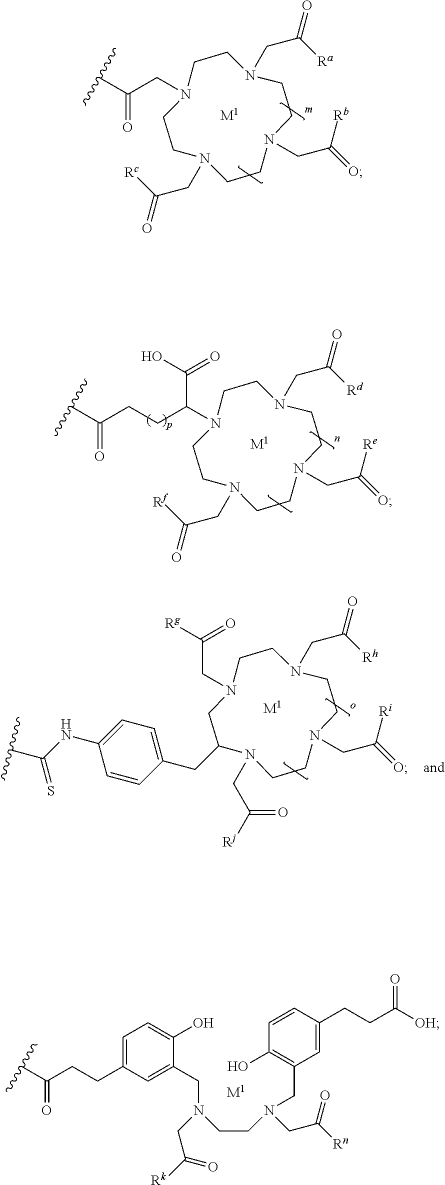

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

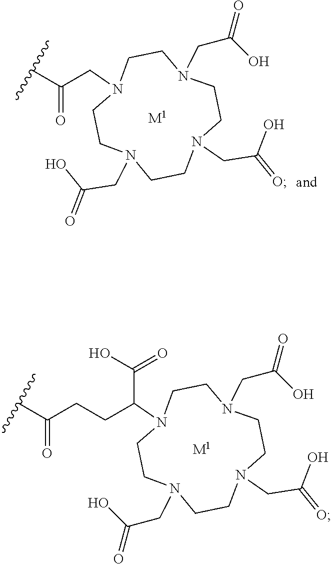

more preferably Ch¹(M¹) is:

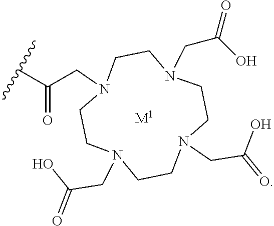

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}—, —NR^{x7}C(O)NR^{x8}—, —NR^{x11}C(O)O—, —NR^{x13}C(O)S—, or —NR^{x19}S(O)₂NR^{x20}—, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is

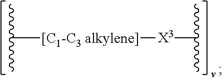

wherein X³ at each occurrence is independently selected from —NH—, —NCH₃—, —O—, and —NHC(O)—, and v is 1, 2, 3, 4, or 5; and
Ch¹(M¹) is selected from:

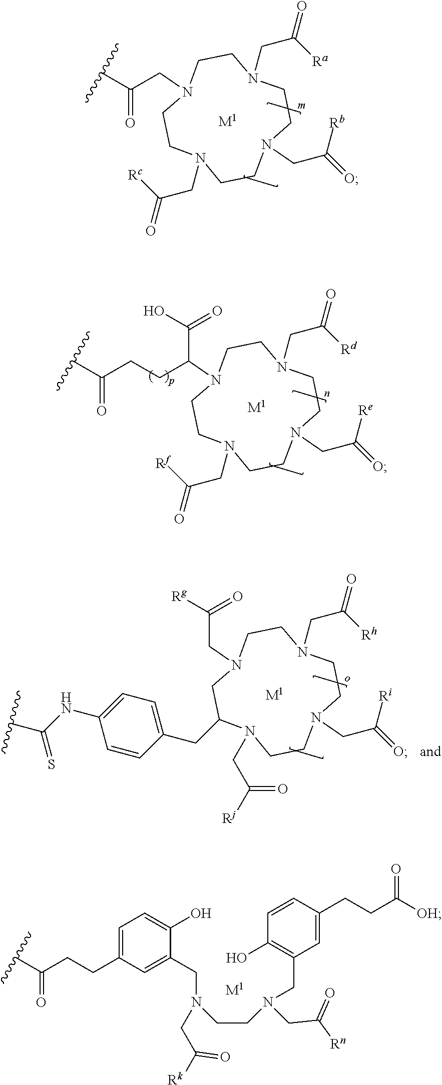

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

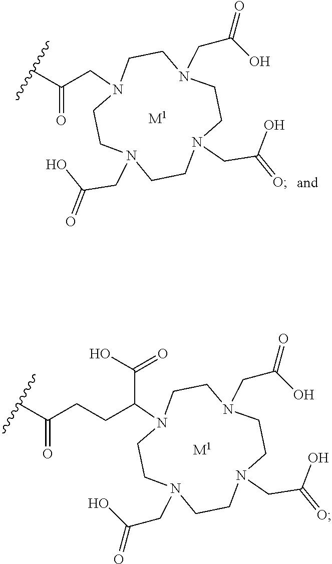

more preferably Ch¹(M¹) is:

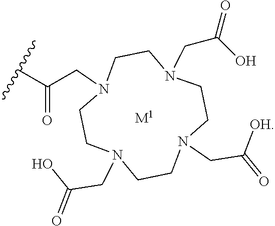

In an embodiment of a compound of Formula (I), or a pharmaceutically acceptable salt thereof,
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—, preferably X² is a bond, —NR^{x2}—, —O—, —NR^{x3}C(O)—, —C(O)NR^{x5}—, —NR^{x7}C(O)NR^{x8}—, —NR^{x11}C(O)O—, —NR^{x13}C(O)S—, or —NR^{x19}S(O)₂NR^{x20}, more preferably X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NH—, —NCH₃C(O)NCH₃—, —NHC(O)O—, —NCH₃C(O)O—, —NHC(O)S—, —NCH₃C(O)S—, —NHS(O)₂NH—, —NCH₃S(O)₂NH—, —NHS(O)₂NCH₃—, or —NCH₃S(O)₂NCH₃—, even more preferably X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, preferably pyrrolidinyl, wherein the heterocyclyl is optionally substituted with C₁-C₆ alkyl, preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, more preferably R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and methyl;
L¹ is selected from

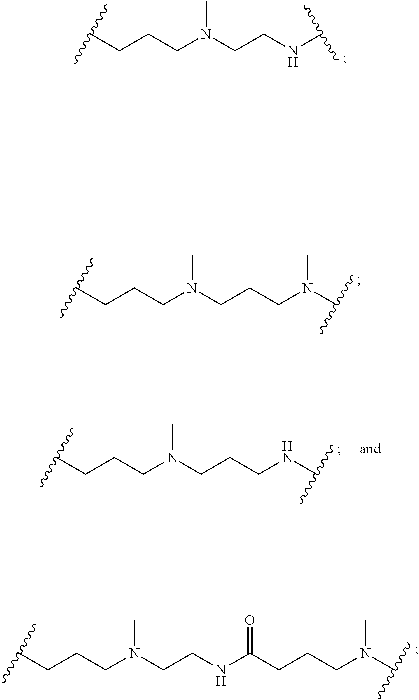

and
Ch¹(M¹) is selected from:

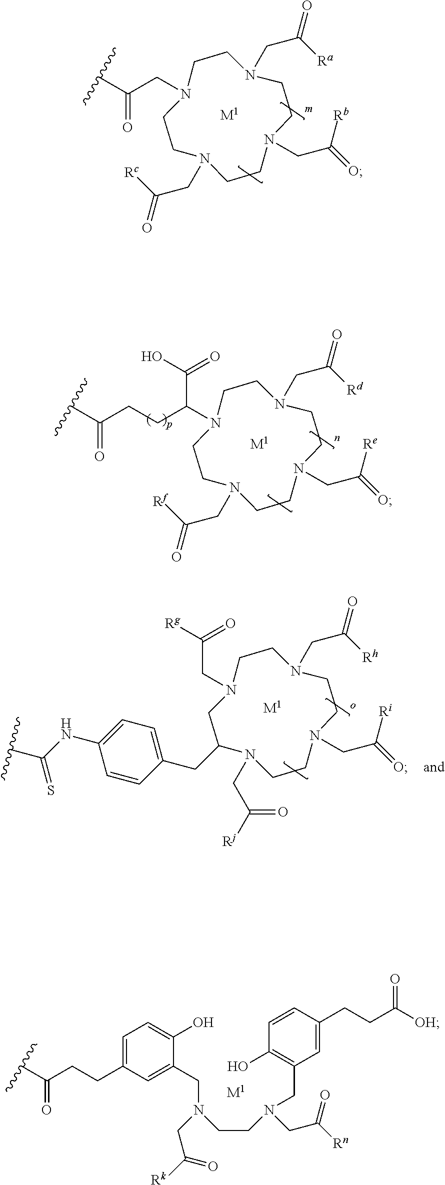

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; m, n, o, and p are each independently 0, 1, or 2; and M¹ is a radionuclide, preferably M¹ is ¹⁷⁷Lu, ²¹²Pb, or ²²⁵Ac; preferably Ch¹(M¹) is selected from:

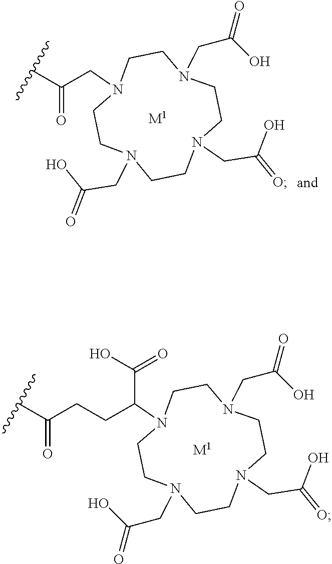

more preferably Ch¹(M¹) is:

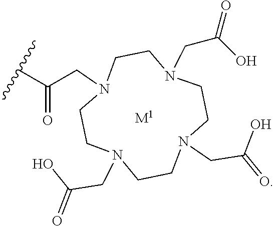

In an embodiment of a compound of Formula (I), or a pharmaceutically acceptable salt thereof, X²-L¹-Ch¹(M¹) is selected from:

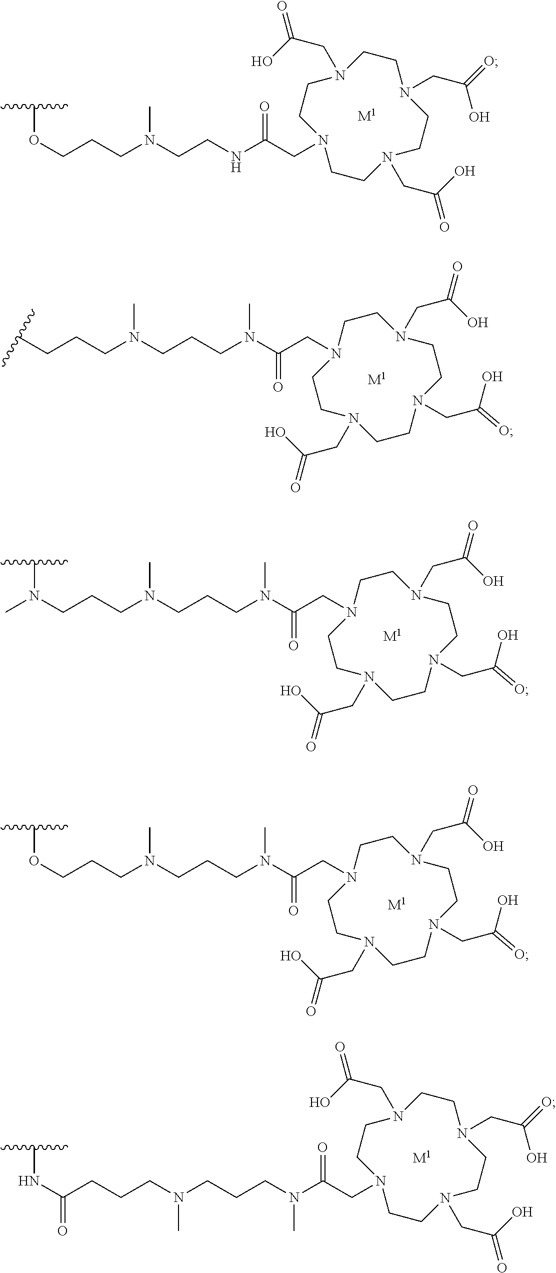

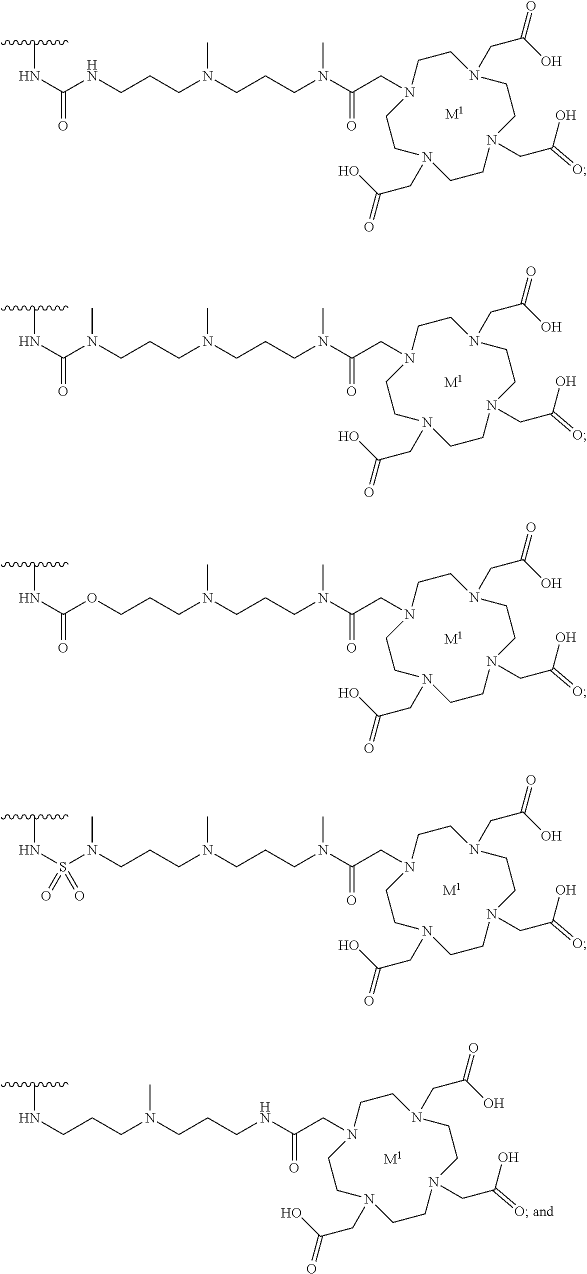

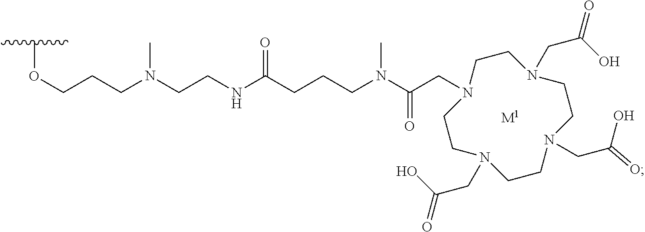

wherein M¹ is a radionuclide, preferably M¹ is ¹⁷⁷Lu, ²¹²Pb, or ²²⁵Ac.

In an embodiment of a compound of Formula (I), or a pharmaceutically acceptable salt thereof, X²-L¹-Ch¹(M¹) is selected from:

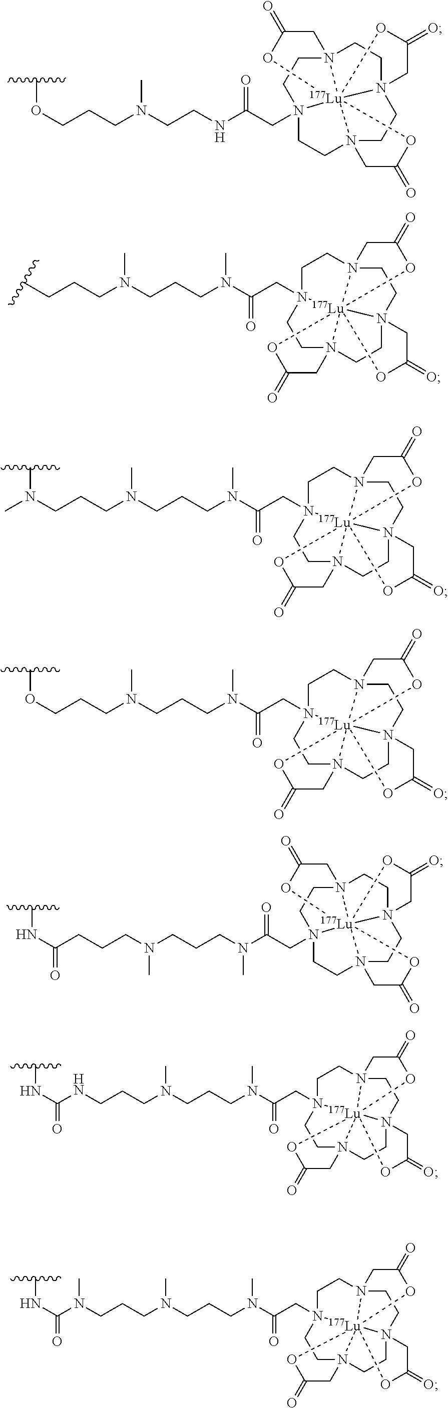

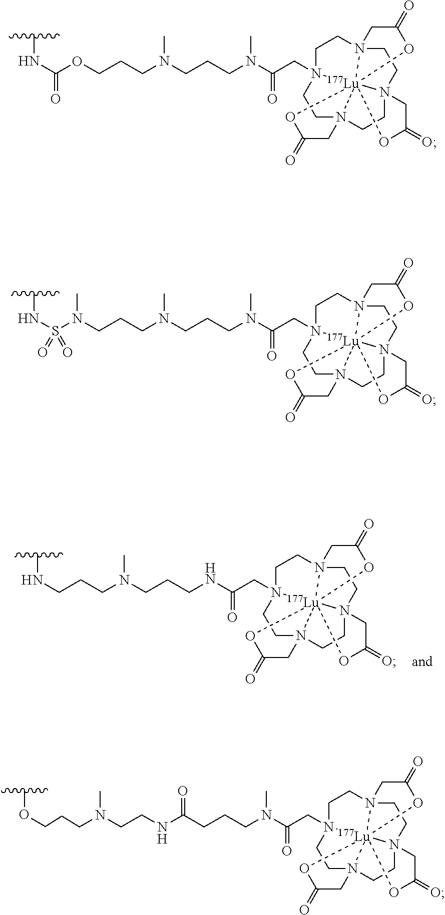

The present invention also provides a compound of Formula (I-a), (I-b), (I-c), (I-d), (I-e), (I-f), (I-g), (I-h), (I-i), (I-j), (I-k), (I-l), (I-m), (I-n), or (I-o):

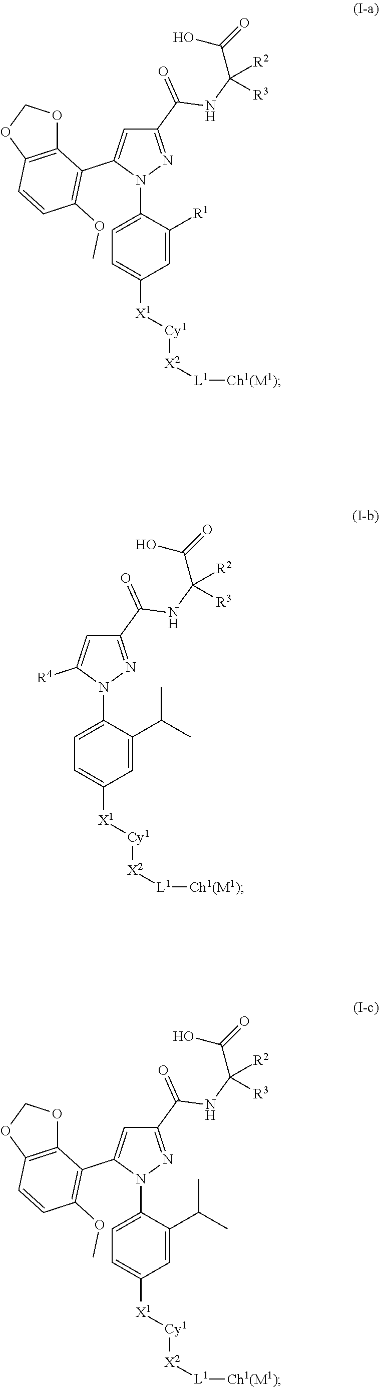

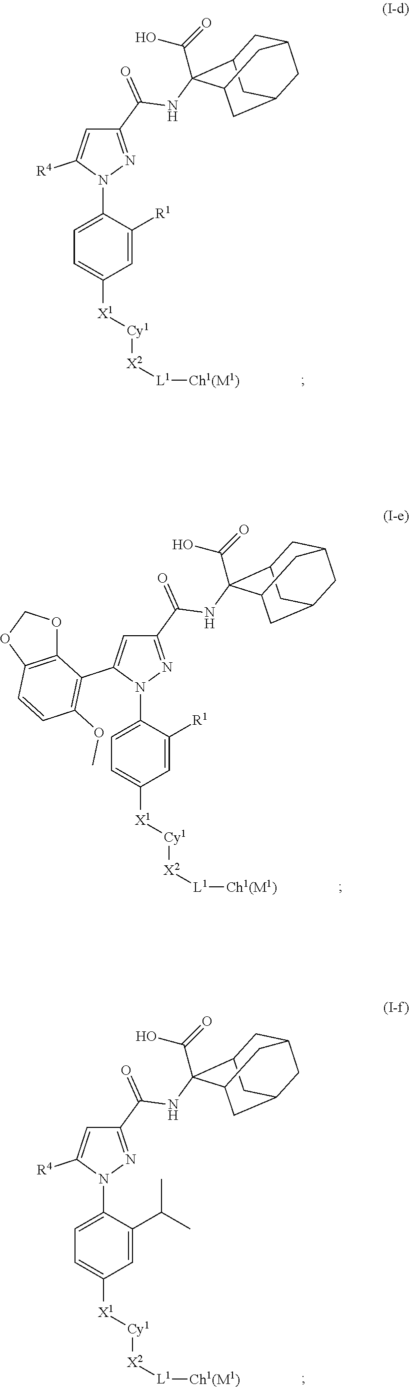

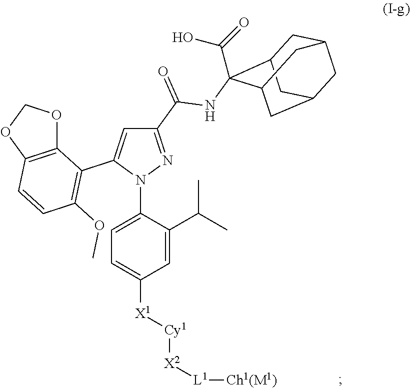

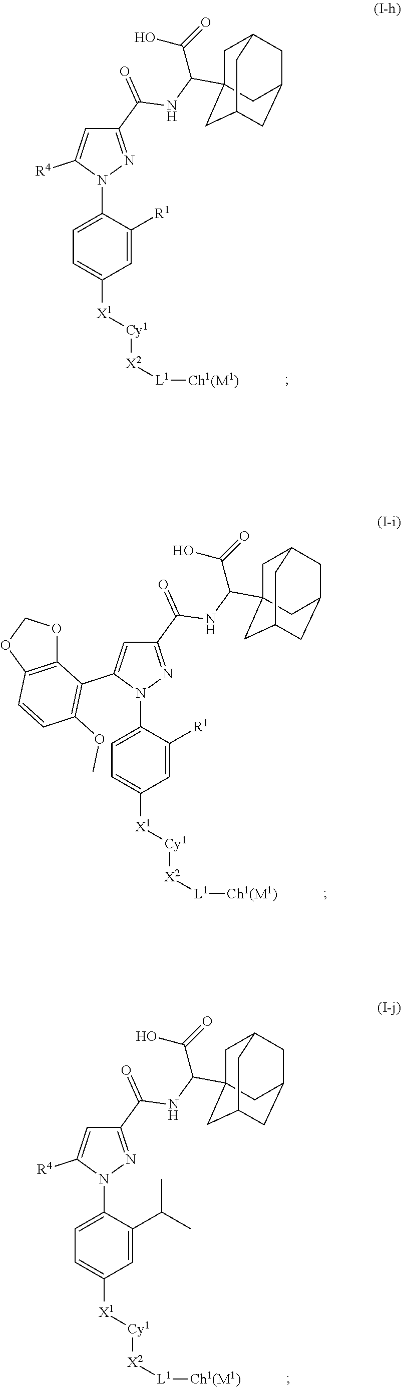

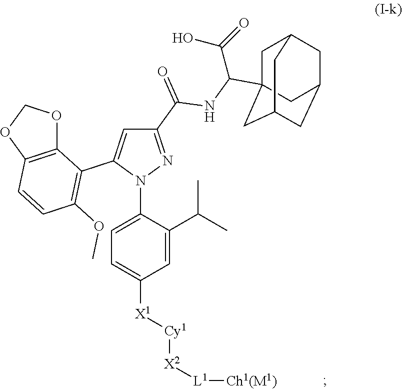

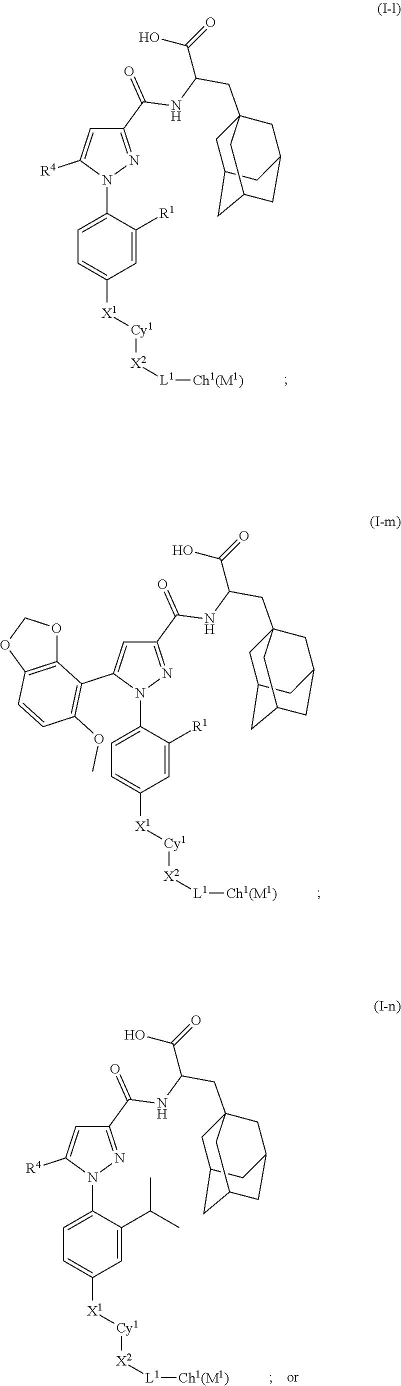

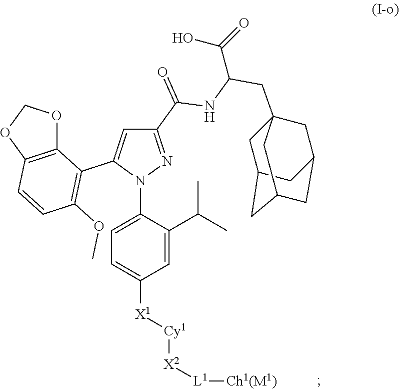

wherein R¹, R², R³, R⁴, X¹, Cy¹, X², L¹, Ch¹, and M¹ are as defined above, or a pharmaceutically acceptable salt thereof.

The present invention also provides a compound of Formula (I-p), (I-q), (I-r), (I-s), or (I-t):

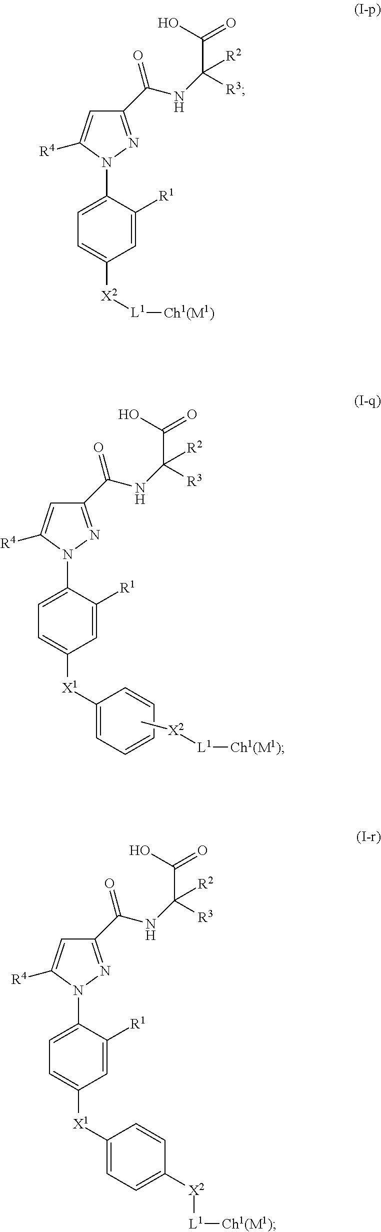

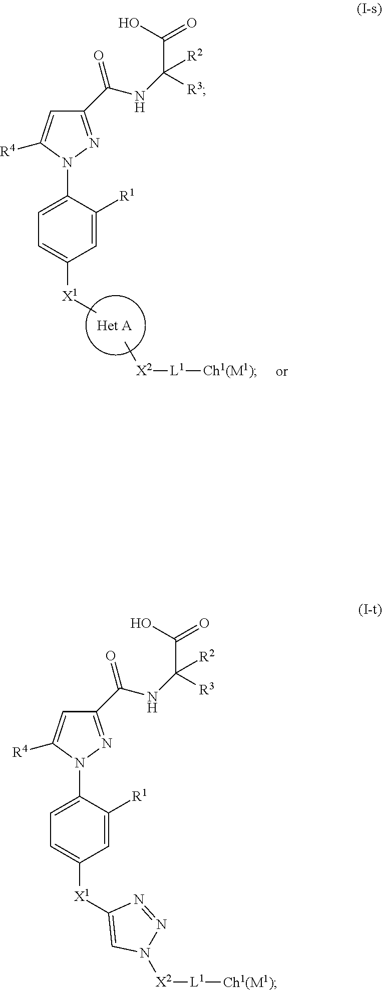

wherein R¹, R², R³, R⁴, X¹, X², L¹, Ch¹, and M¹ are as defined above, and Het A is 5- or 6-membered heteroaryl containing 1-3 heteroatoms independently selected from N, O, and S, or a pharmaceutically acceptable salt thereof.

The present invention also provides a compound of Formula (I-a-1), (I-b-1), (I-c-1), (I-d-1), (I-e-1), (I-f-1), or (I-g-1):

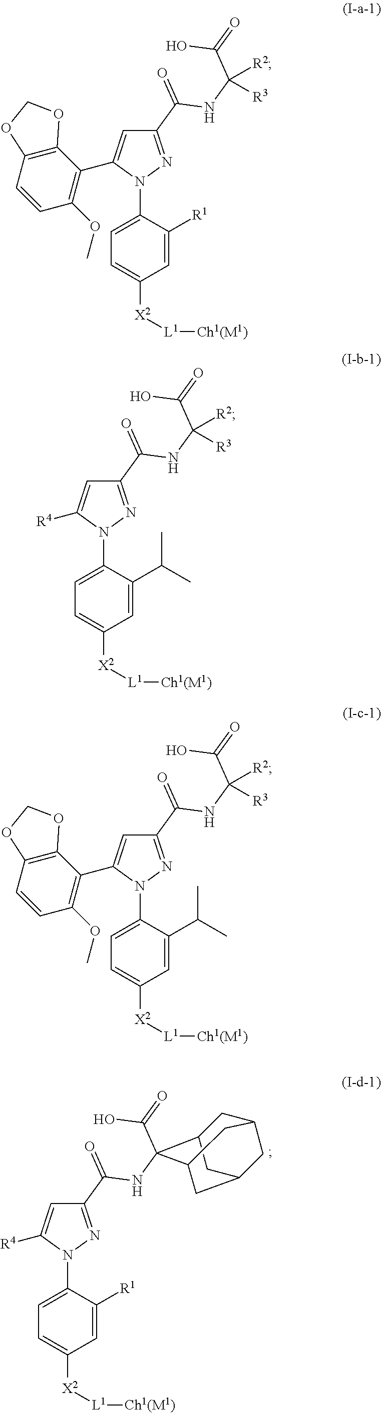

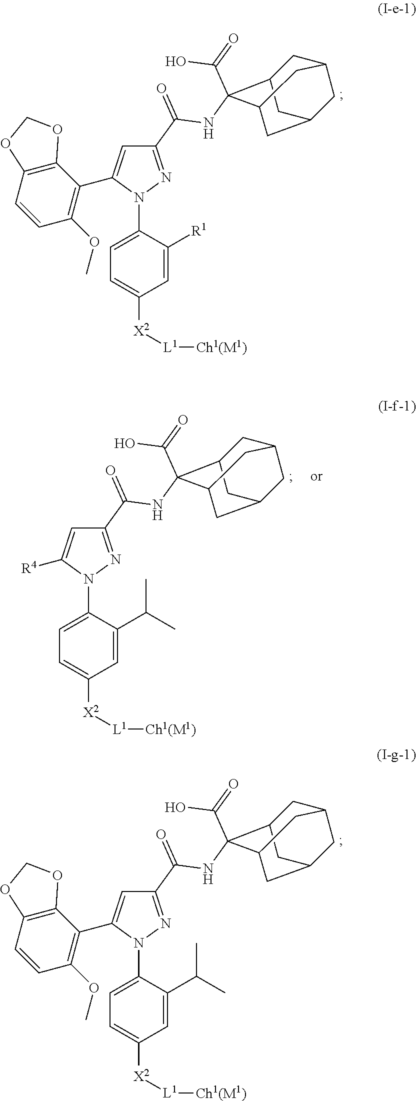

wherein R¹, R², R³, R⁴, X², L¹, Ch¹, and M¹ are as defined above, or a pharmaceutically acceptable salt thereof.

The present invention also provides a compound of Formula (I-a-2), (I-b-2), (I-c-2), (I-d-2), (I-e-2), (I-f-2), or (I-g-2):

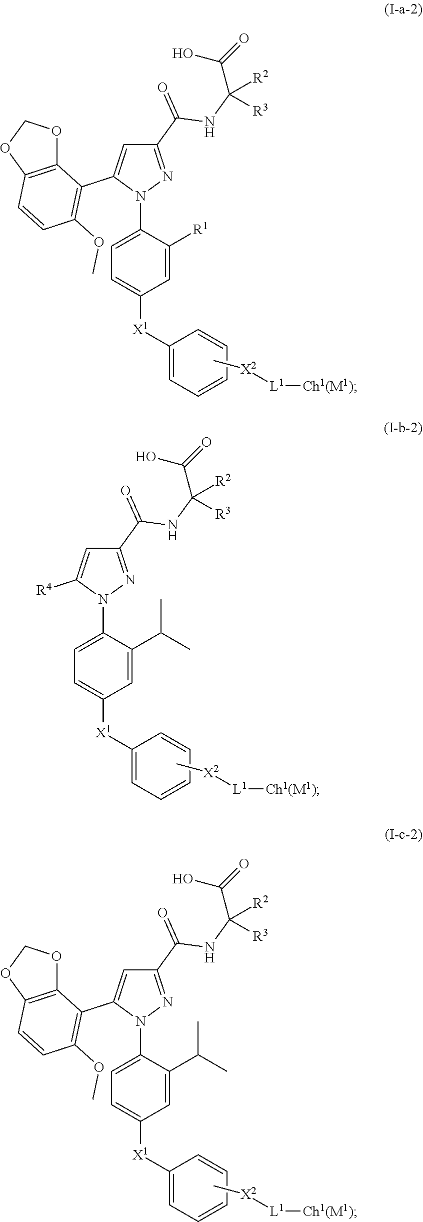

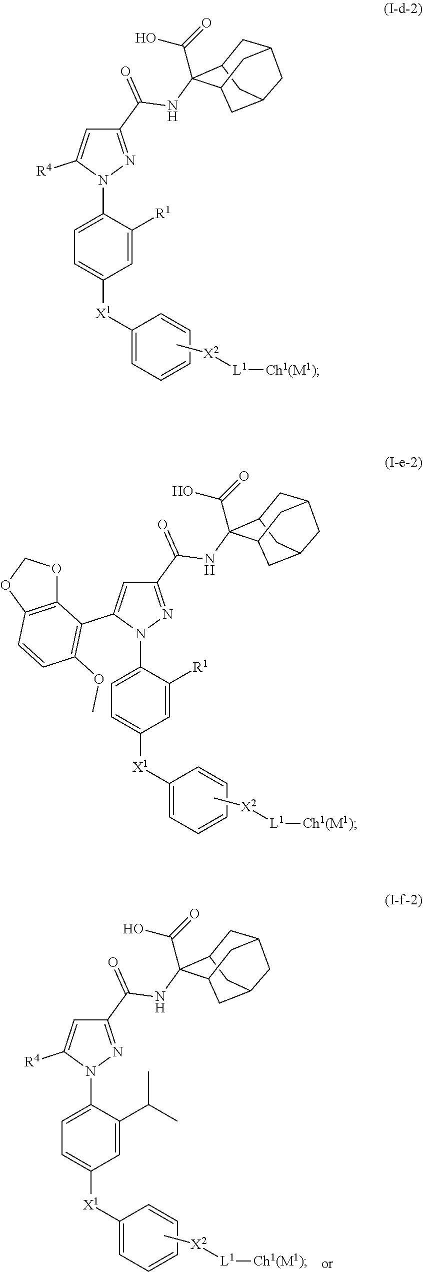

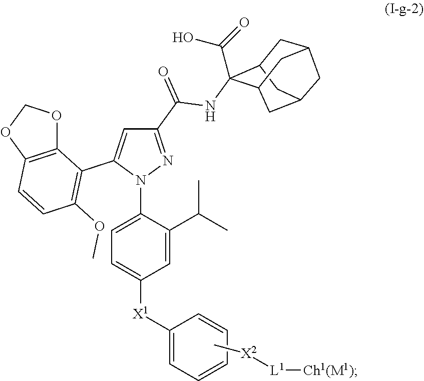

wherein R¹, R², R³, R⁴, X¹, X², L¹, Ch¹, and M¹ are as defined above, or a pharmaceutically acceptable salt thereof.

The present invention also provides a compound of Formula (I-a-3), (I-b-3), (I-c-3), (I-d-3), (I-e-3), (I-f-3), or (I-g-3):

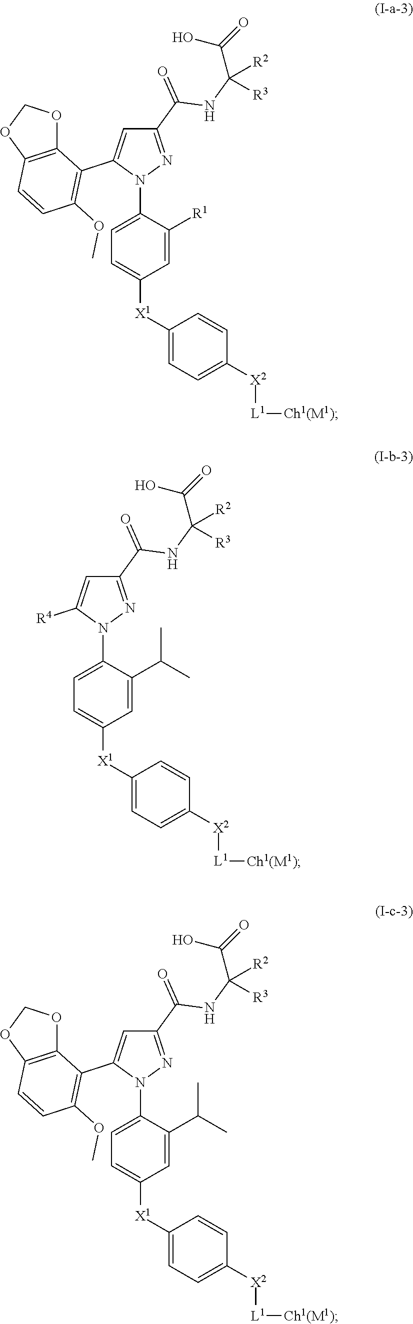

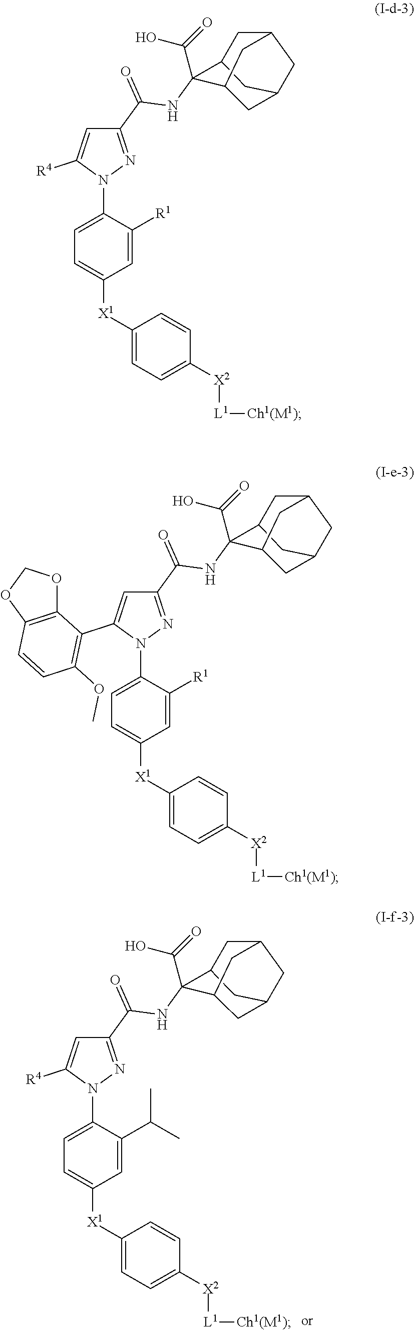

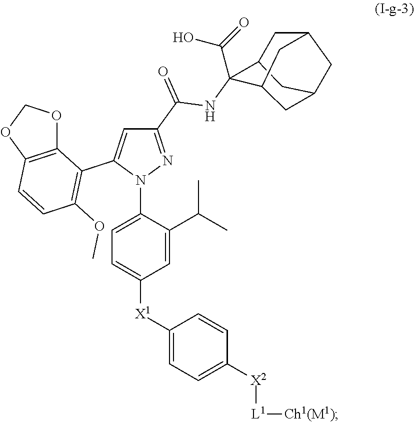

wherein R¹, R², R³, R⁴, X¹, X², L¹, Ch¹, and M¹ are as defined above, or a pharmaceutically acceptable salt thereof.

The present invention also provides a compound of Formula (I-a-4), (I-b-4), (I-c-4), (I-d-4), (I-e-4), (I-f-4), or (I-g-4):

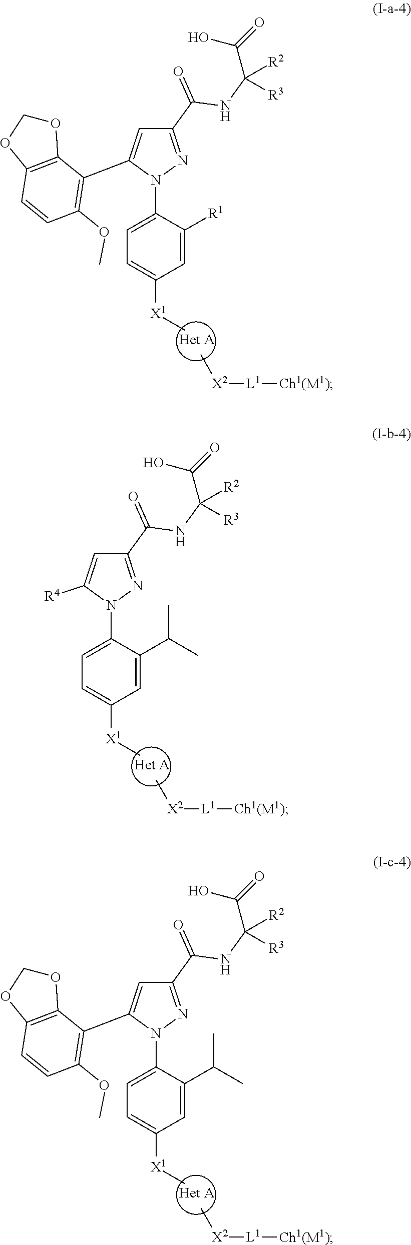

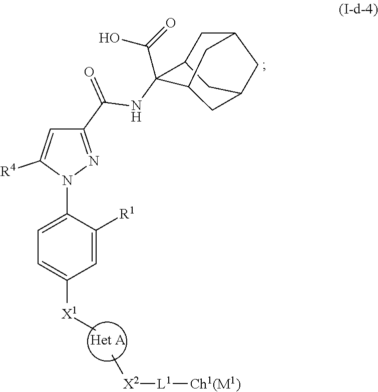

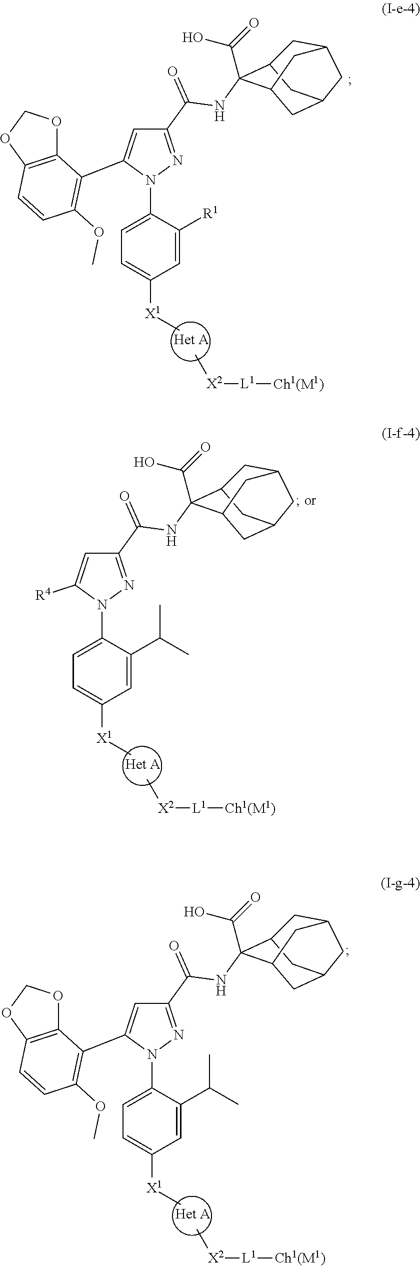

wherein R¹, R², R³, R⁴, X¹, X², L¹, Ch¹, and M¹ are as defined above, and Het A is 5- or 6-membered heteroaryl containing 1-3 heteroatoms independently selected from N, O, and S, or a pharmaceutically acceptable salt thereof.

The present invention also provides a compound of Formula (I-a-5), (I-b-5), (I-c-5), (I-d-5), (I-e-5), (I-f-5), or (I-g-5):

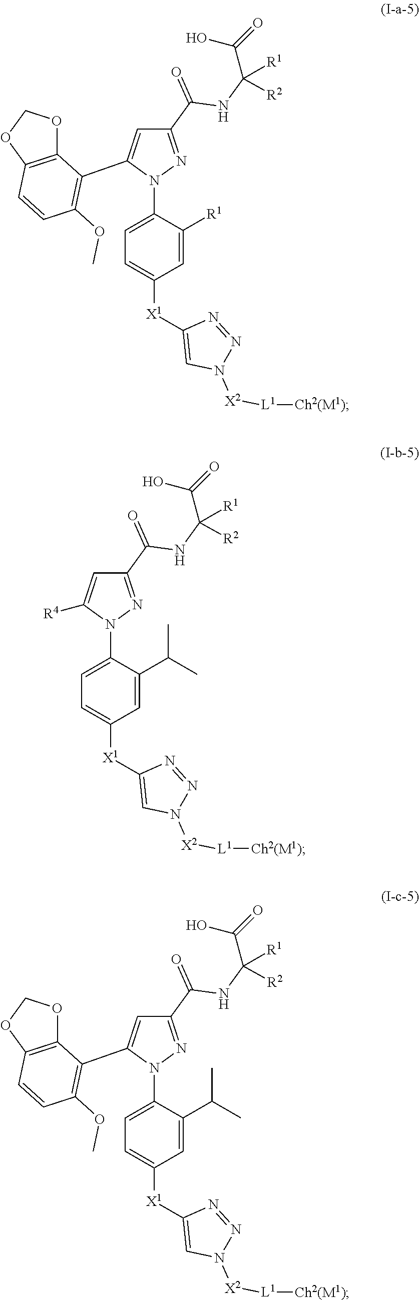

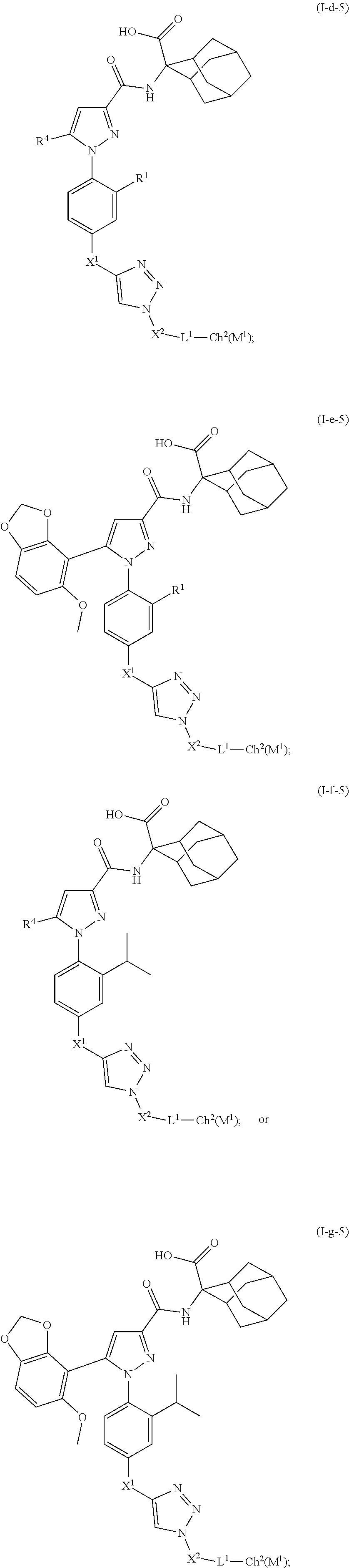

wherein R¹, R², R³, R⁴, X¹, X², L¹, Ch¹, and M¹ are as defined above, or a pharmaceutically acceptable salt thereof.

The present invention also provides a compound of Formula (I-g-1-a), (I-g-1-b), (I-g-1-c), (I-g-1-d), (I-g-1-e), (I-g-1-f), (I-g-1-g), (I-g-1-h), or (I-g-1-i):

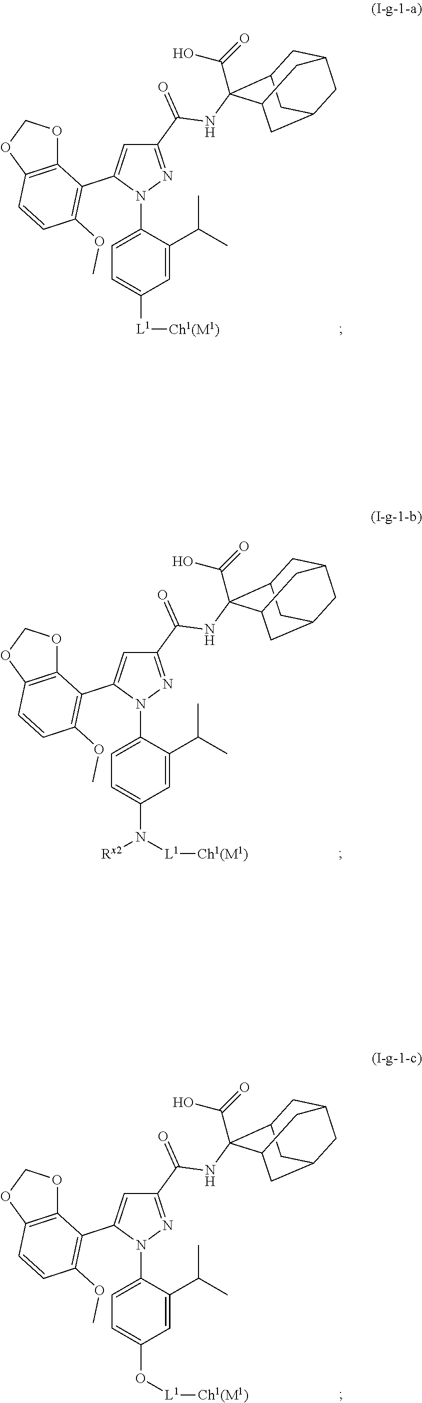

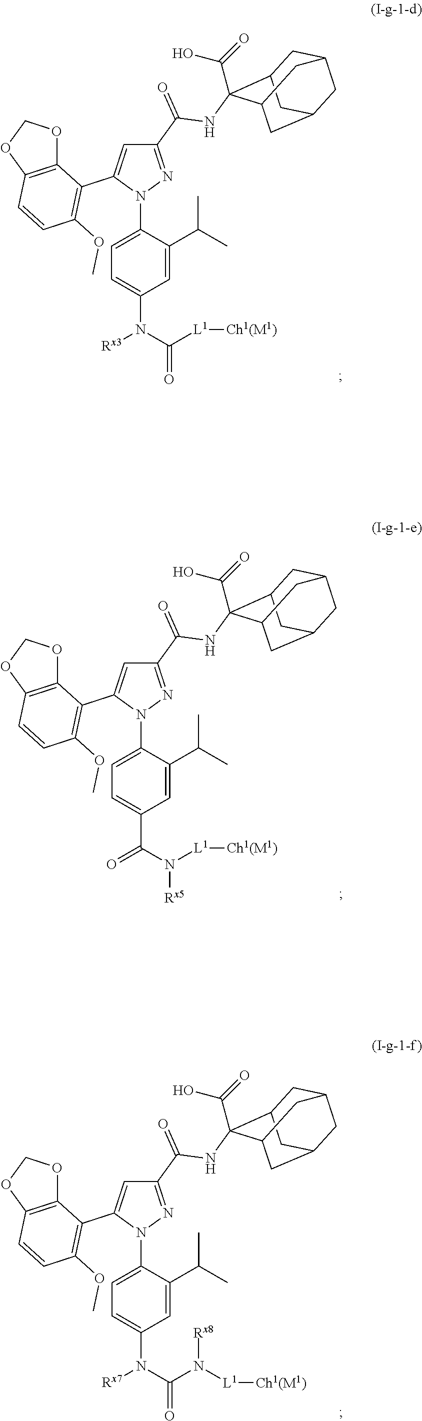

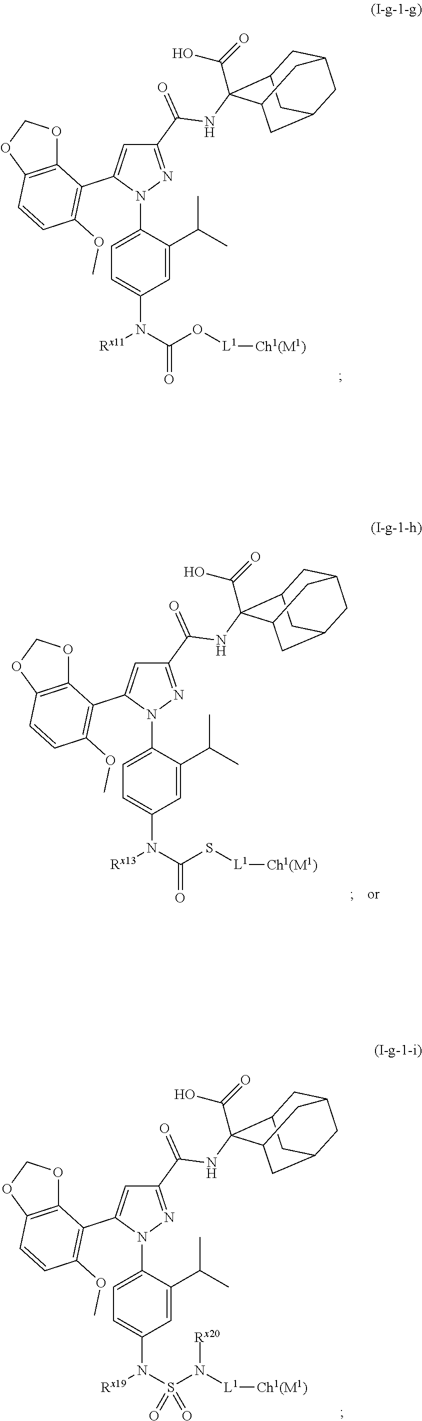

wherein R^{x2}, R^{x3}, R^{x5}, R^{x7}, R^{x8}, R^{x11}, R^{x13}, R^{x19}, R^{x20}, L¹, Ch¹, and M¹ are as defined above, or a pharmaceutically acceptable salt thereof; preferably R^{x2}, R^{x2}, R^{x3}, R^{x5}, R^{x7}, R^{x8}, R^{x11}, R^{x13}, R^{x19}, and R^{x20} are each independently selected from H and C₁-C₆ alkyl, more preferably R^{x2}, R^{x3}, R^{x5}, R^{x7}, R^{x8}, R^{x11}, R^{x13}, R^{x19}, and R^{x20} are each independently selected from H and methyl.

The present invention also provides a compound of Formula (I-g-2-a), (I-g-2-b), (I-g-2-c), (I-g-2-d), (I-g-2-e), (I-g-2-f), (I-g-2-g), (I-g-2-h), or (I-g-2-i):

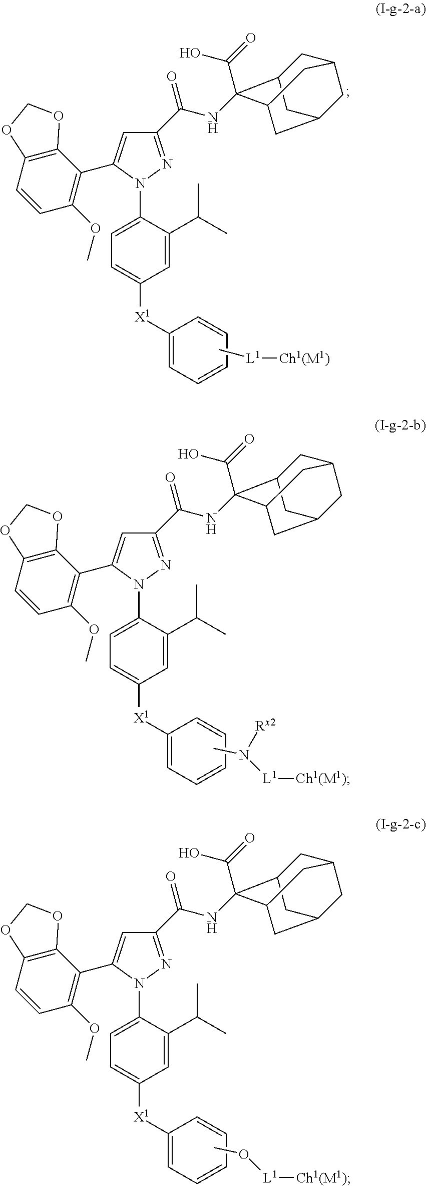

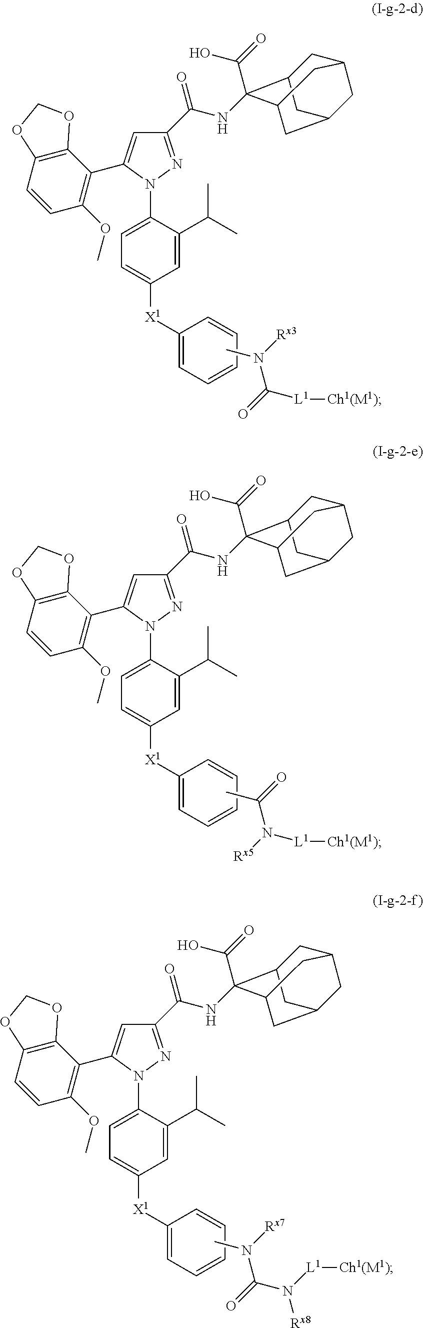

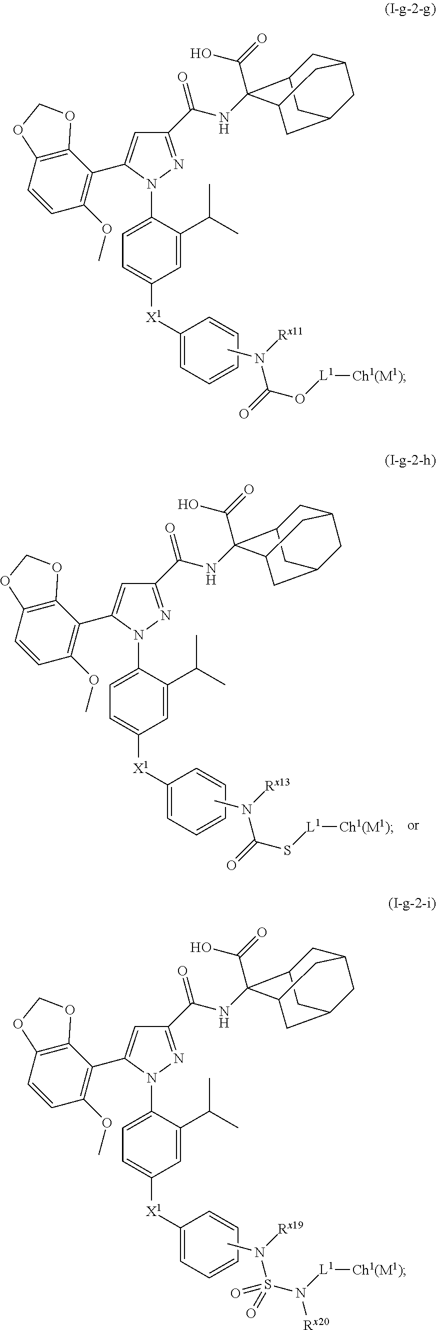

wherein R^{x2}, R^{x3}, R^{x5}, R^{x7}, R^{x8}, R^{x11}, R^{x13}, R^{x19}, R^{x20}, X¹, L¹, Ch¹, and M¹ are as defined above, or a pharmaceutically acceptable salt thereof, preferably R^{x2}, R^{x2}, R^{x3}, R^{x5}, R^{x7}, R^{x8}, R^{x11}, R^{x13}, R^{x19}, and R^{x20} are each independently selected from H and C₁-C₆ alkyl, more preferably R^{x2}, R^{x3}, R^{x5}, R^{x7}, R^{x8}, R^{x11}, R^{x13}, R^{x19}, and R^{x20} are each independently selected from H and methyl.

The present invention also provides a compound of Formula (I-g-3-a), (I-g-3-b), (I-g-3-c), (I-g-3-d), (I-g-3-e), (I-g-3-f), (I-g-3-g), (I-g-3-h), or (I-g-3-i):

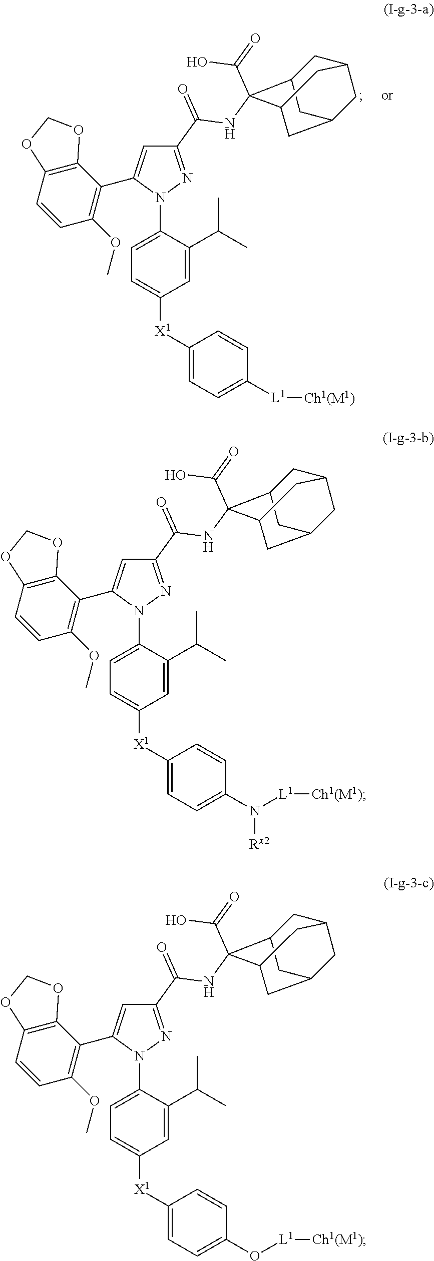

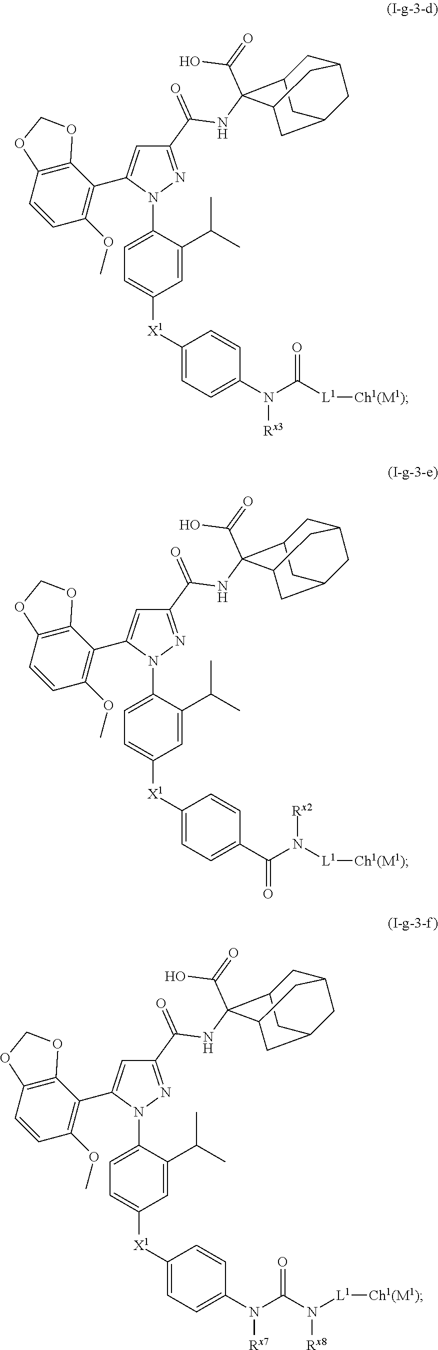

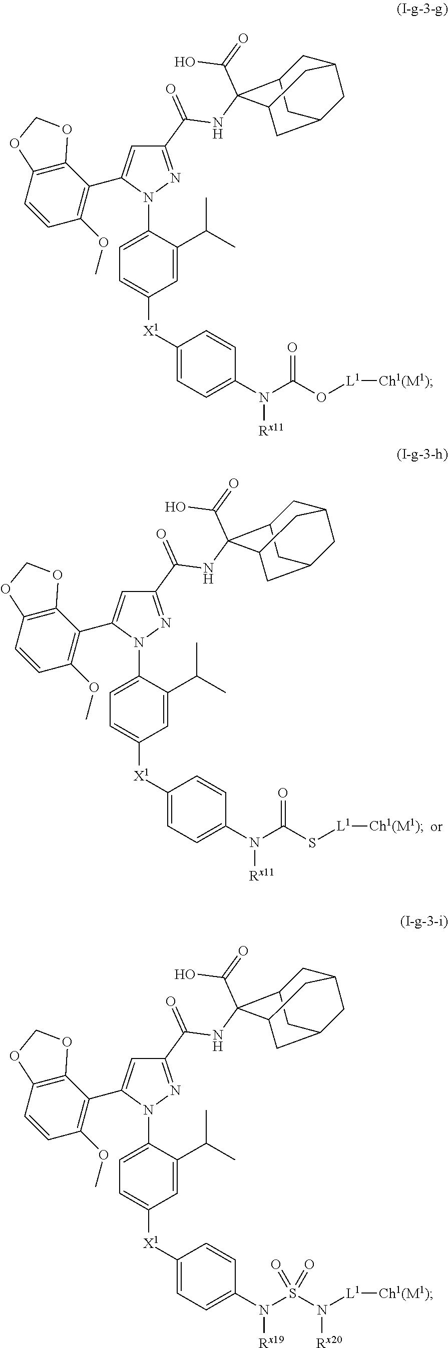

wherein R^{x2}, R^{x3}, R^{x5}, R^{x7}, R^{x8}, R^{x11}, R^{x13}, R^{x19}, R^{x20}, X¹, L¹, Ch¹, and M¹ are as defined above, or a pharmaceutically acceptable salt thereof; preferably R^{x2}, R^{x2}, R^{x3}, R^{x5}, R^{x7}, R^{x8}, R^{x11}, R^{x13}, R^{x19}, and R^{x20} are each independently selected from H and C₁-C₆ alkyl, more preferably R^{x2}, R^{x3}, R^{x5}, R^{x7}, R^{x8}, R^{x11}, R^{x13}, R^{x19}, and R^{x20} are each independently selected from H and methyl.

The present invention also provides a compound of Formula (I-g-4-a), (I-g-4-b), (I-g-4-c), (I-g-4-d), (I-g-4-e), (I-g-4-f), (I-g-4-g), (I-g-4-h), or (I-g-4-i):

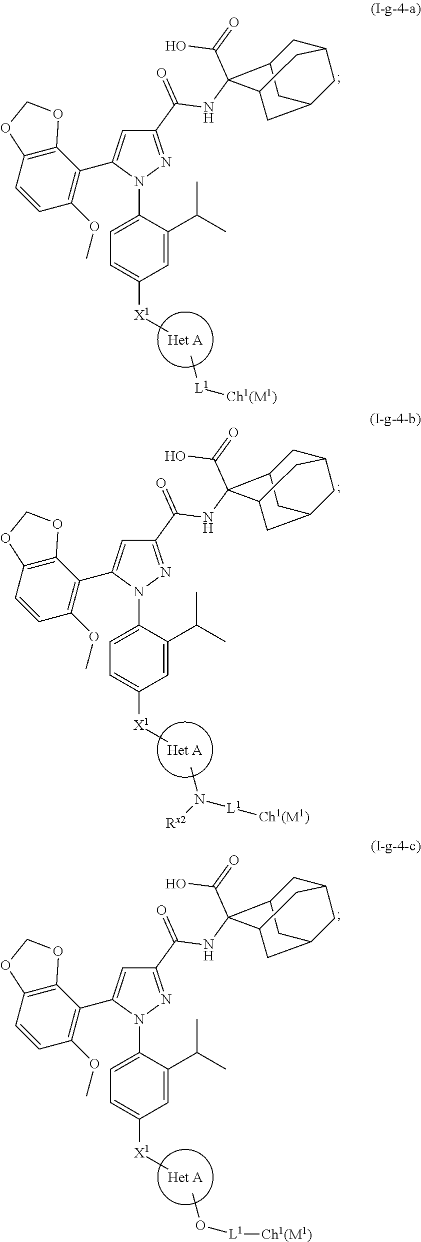

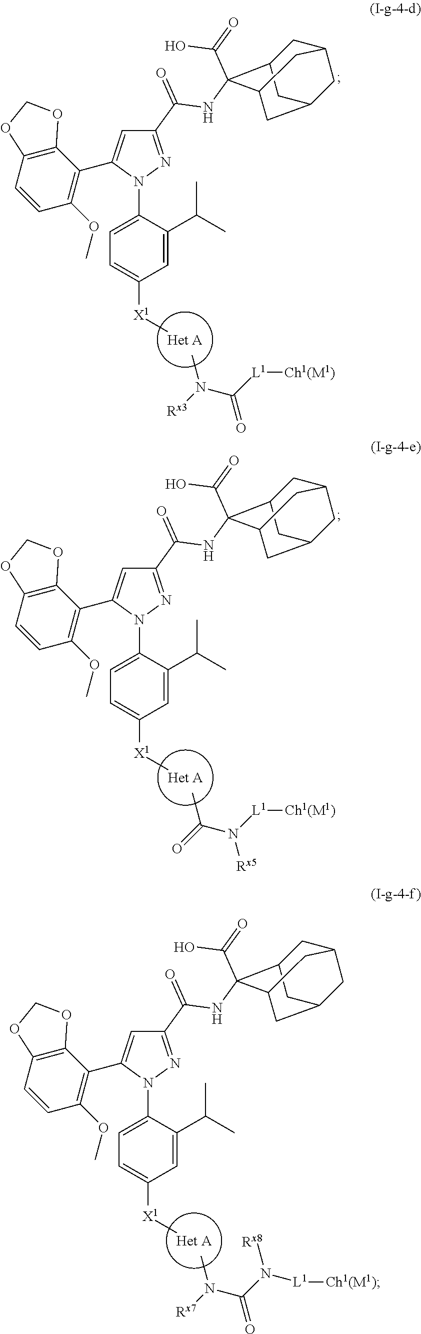

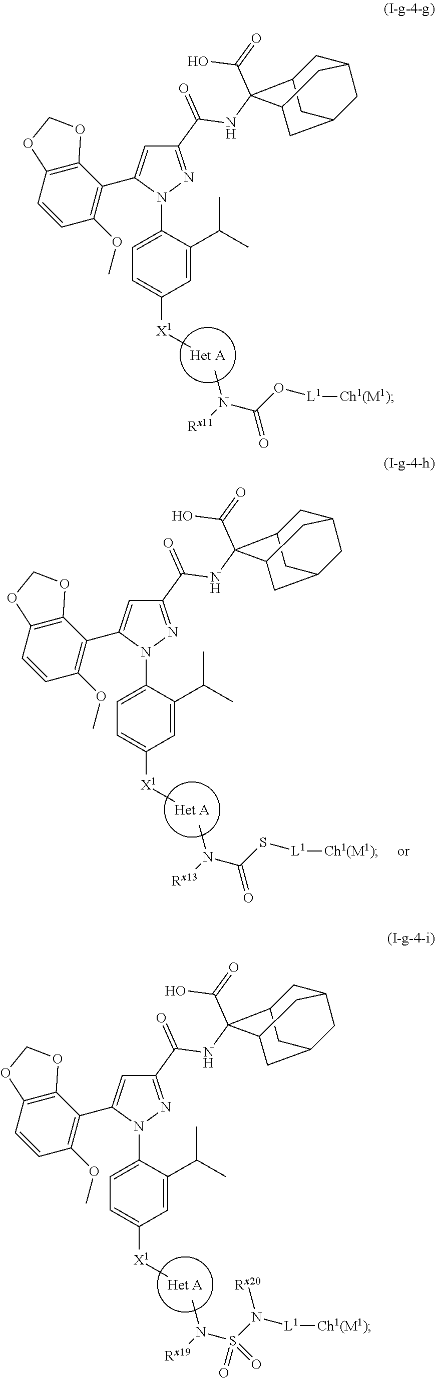

wherein R^{x2}, R^{x3}, R^{x5}, R^{x7}, R^{x8}, R^{x11}, R^{x13}, R^{x19}, R^{x20}, X¹, L¹, Ch¹, and M¹ are as defined above, and Het A is 5- or 6-membered heteroaryl containing 1-3 heteroatoms independently selected from N, O, and S, or a pharmaceutically acceptable salt thereof; preferably R^{x2}, R^{x2}, R^{x3}, R^{x5}, R^{x7}, R^{x8}, R^{x11}, R^{x13}, R^{x19}, and R^{x20} are each independently selected from H and C₁-C₆ alkyl, more preferably R^{x2}, R^{x3}, R^{x5}, R^{x7}, R^{x8}, R^{x11}, R^{x13}, R^{x19}, and R^{x20} are each independently selected from H and methyl.

The present invention also provides a compound of Formula (I-g-5-a):

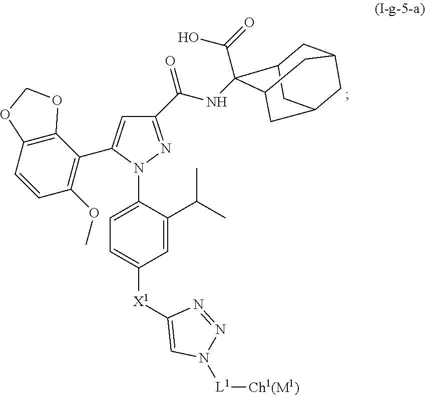

wherein X¹, L¹, Ch¹, and M¹ are as defined above, or a pharmaceutically acceptable salt thereof.

The present invention also provides a compound of Formula (I-ChM^{A}), (I-ChM^{B}), (I-ChM^{A1}), (I-ChM^{B1}), (I-ChM^{A2}), (I-ChM^{B2}), (I-g-ChM^{A2}), or (I-g-ChMB2):

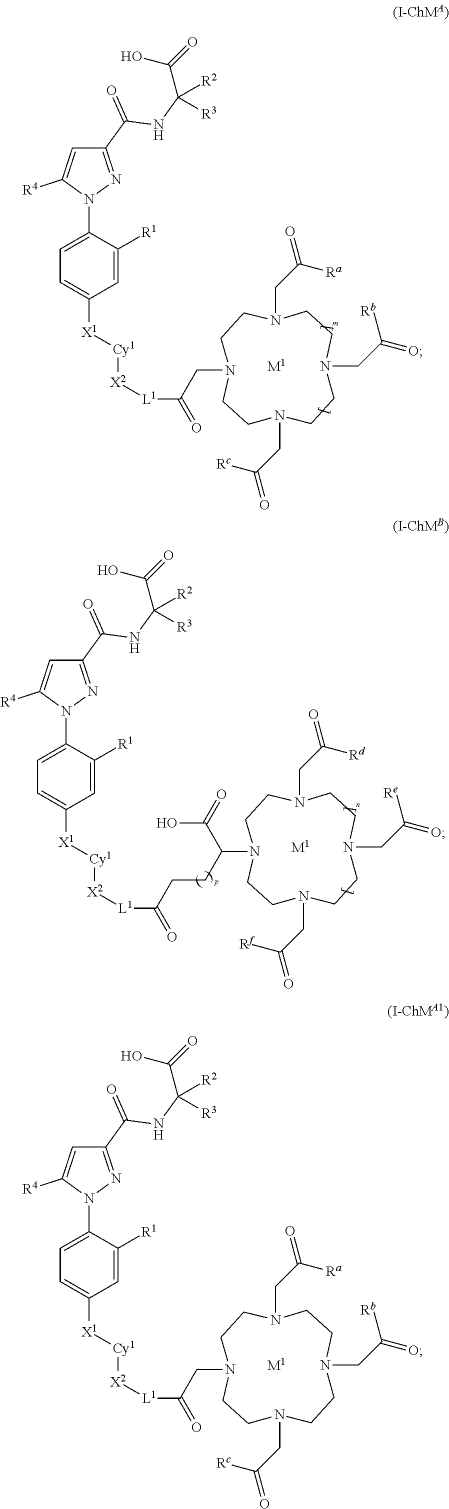

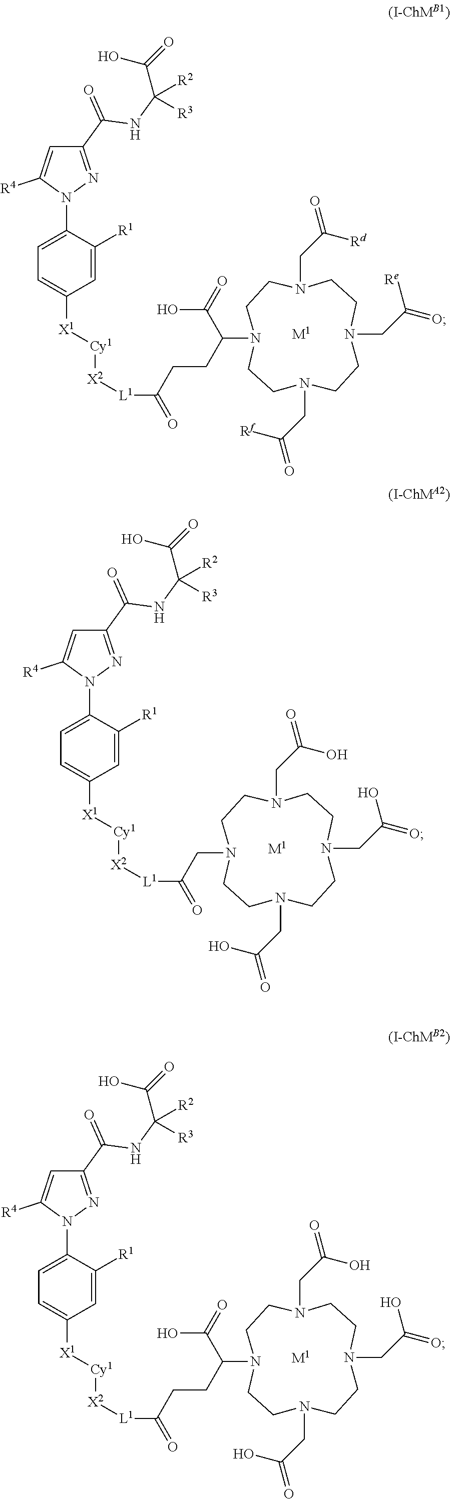

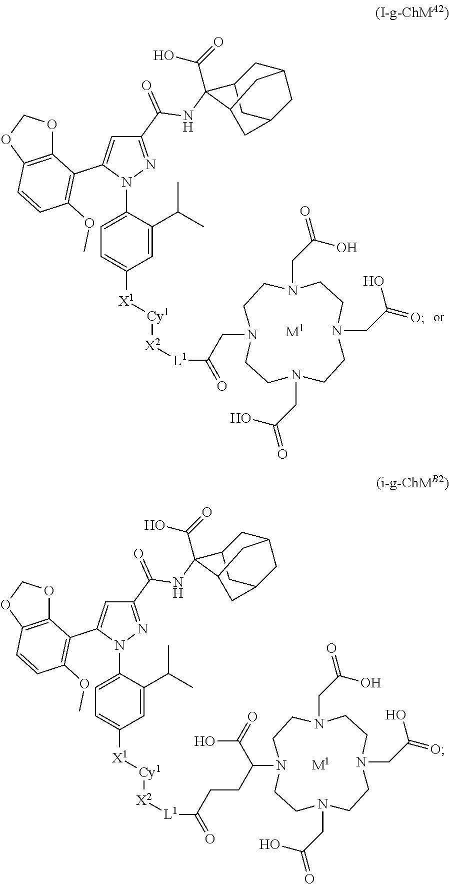

wherein R¹, R², R³, R⁴, X¹, Cy¹, X², L¹, M¹, R^{a}, R^{b}, R^{c}, R^{d}, R^{e}, R^{f}, m, n, and p are as defined above, or a pharmaceutically acceptable salt thereof.

The present invention also provides a compound selected from:

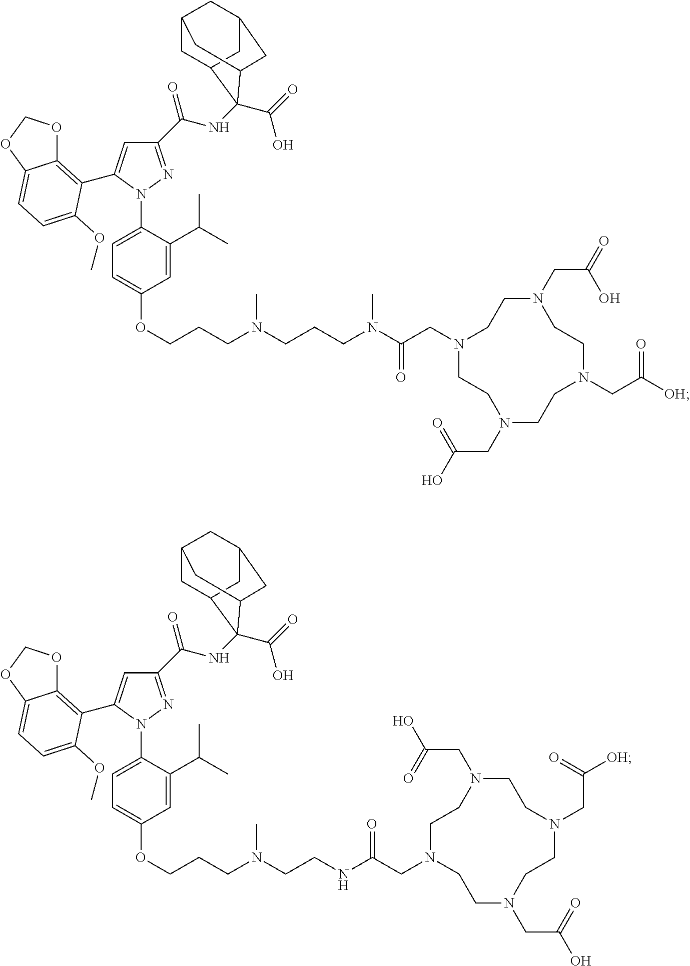 [SMILES: COc1ccc2c(c1-c1cc(C(=O)NC3(C(=O)O)C4CC5CC(C4)CC3C5)nn1-c1ccc(OCCCN(C)CCCN(C)C(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(OCCCN(C)CCNC(=O)CN3CCN(CC(=O)O)CCN(CC(=O)O)CCN(CC(=O)O)CC3)cc1C(C)C)OCO2]

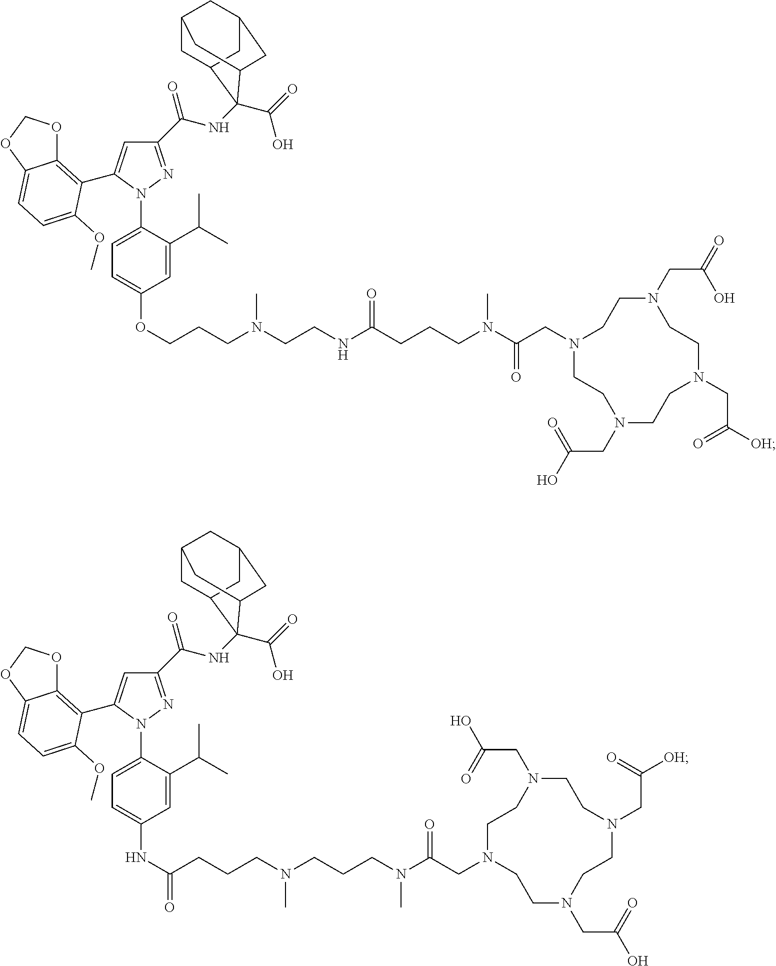 [SMILES: COc1ccc2c(c1-c1cc(C(=O)NC3(C(=O)O)C4CC5CC(C4)CC3C5)nn1-c1ccc(OCCCN(C)CCNC(=O)CCCN(C)C(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(NC(=O)CCCN(C)CCCN(C)C(=O)CN3CCN(CC(=O)O)CCN(CC(=O)O)CCN(CC(=O)O)CC3)cc1C(C)C)OCO2]

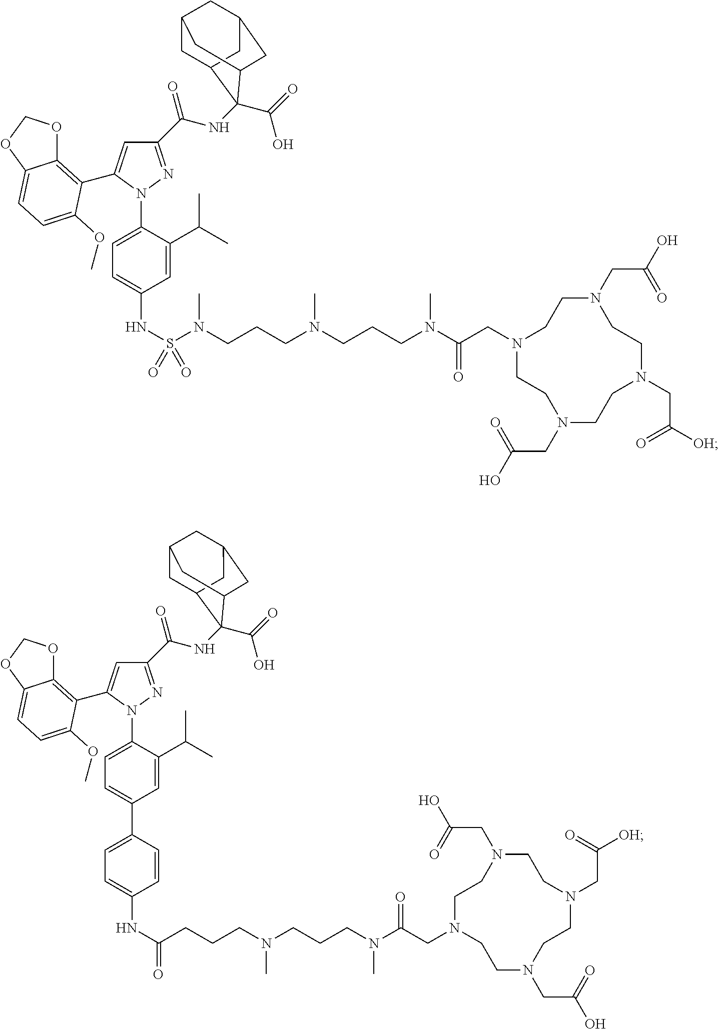 [SMILES: COc1ccc2c(c1-c1cc(C(=O)NC3(C(=O)O)C4CC5CC(C4)CC3C5)nn1-c1ccc(NS(=O)(=O)N(C)CCCN(C)CCCN(C)C(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(-c3ccc(NC(=O)CCCN(C)CCCN(C)C(=O)CN4CCN(CC(=O)O)CCN(CC(=O)O)CCN(CC(=O)O)CC4)cc3)cc1C(C)C)OCO2]

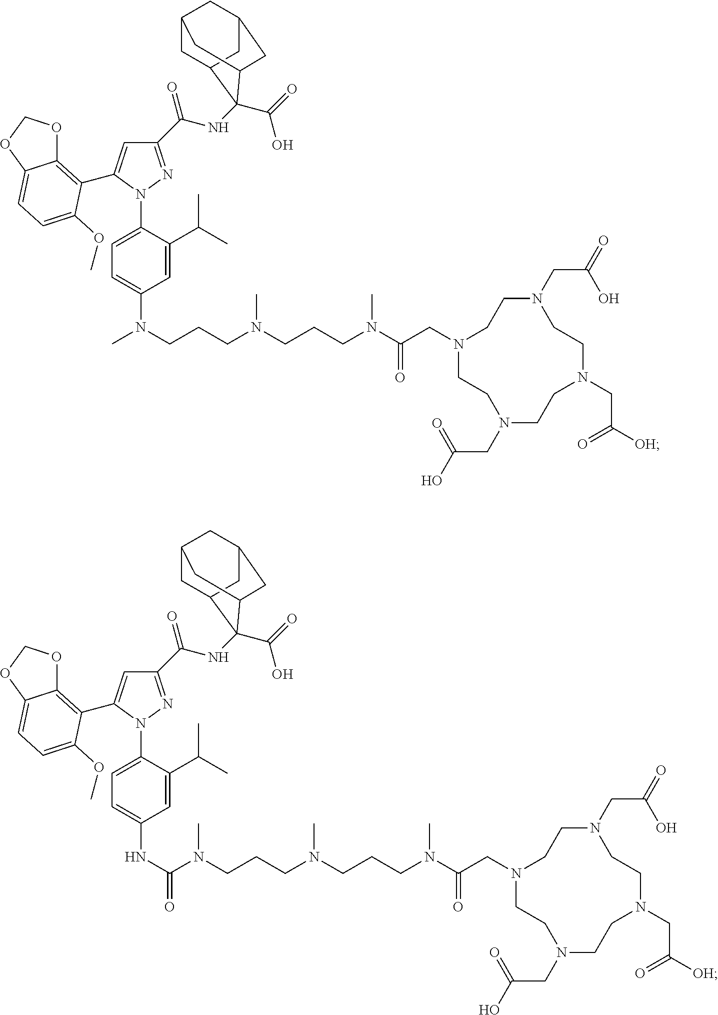 [SMILES: COc1ccc2c(c1-c1cc(C(=O)NC3(C(=O)O)C4CC5CC(C4)CC3C5)nn1-c1ccc(N(C)CCCN(C)CCCN(C)C(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(NC(=O)N(C)CCCN(C)CCCN(C)C(=O)CN3CCN(CC(=O)O)CCN(CC(=O)O)CCN(CC(=O)O)CC3)cc1C(C)C)OCO2]

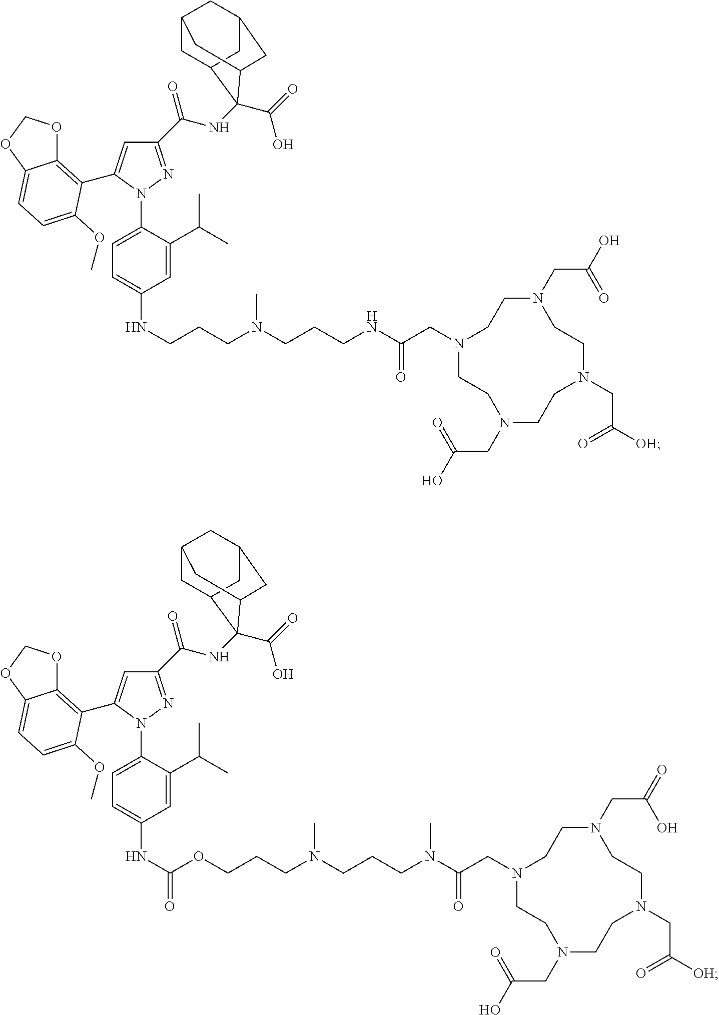 [SMILES: COc1ccc2c(c1-c1cc(C(=O)NC3(C(=O)O)C4CC5CC(C4)CC3C5)nn1-c1ccc(NCCCN(C)CCCNC(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(NC(=O)OCCCN(C)CCCN(C)C(=O)CN3CCN(CC(=O)O)CCN(CC(=O)O)CCN(CC(=O)O)CC3)cc1C(C)C)OCO2]

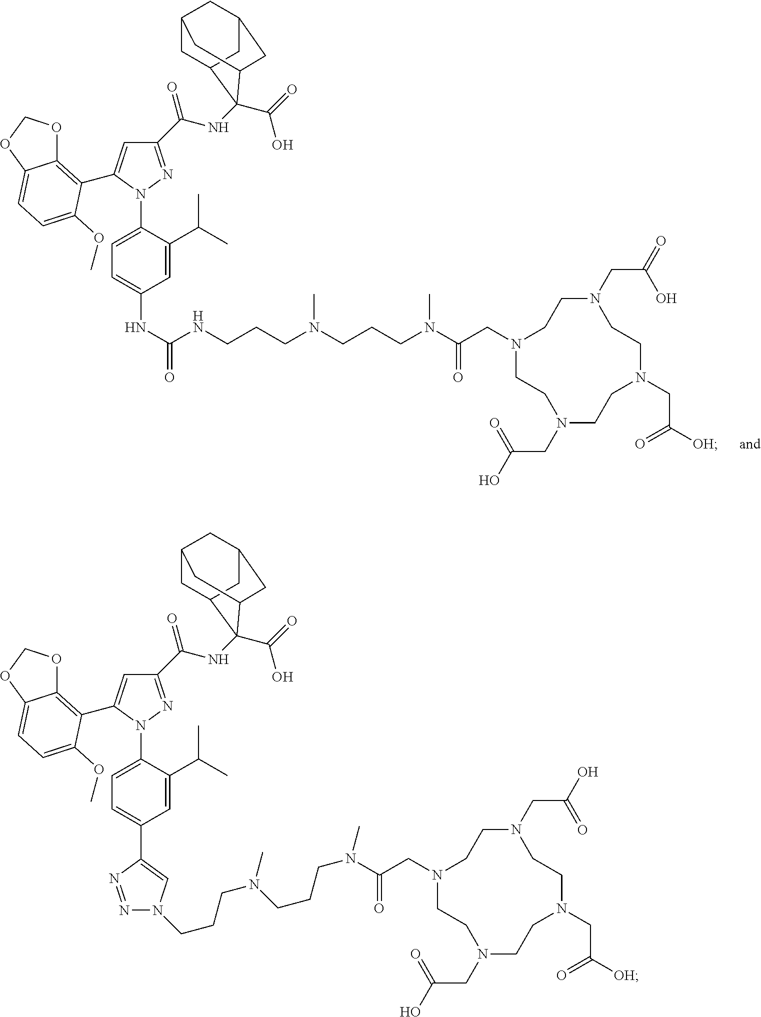 [SMILES: COc1ccc2c(c1-c1cc(C(=O)NC3(C(=O)O)C4CC5CC(C4)CC3C5)nn1-c1ccc(NC(=O)NCCCN(C)CCCN(C)C(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(-c3cn(CCCN(C)CCCN(C)C(=O)CN4CCN(CC(=O)O)CCN(CC(=O)O)CCN(CC(=O)O)CC4)nn3)cc1C(C)C)OCO2]

or a pharmaceutically acceptable salt thereof.

The present invention also provides a compound selected from:

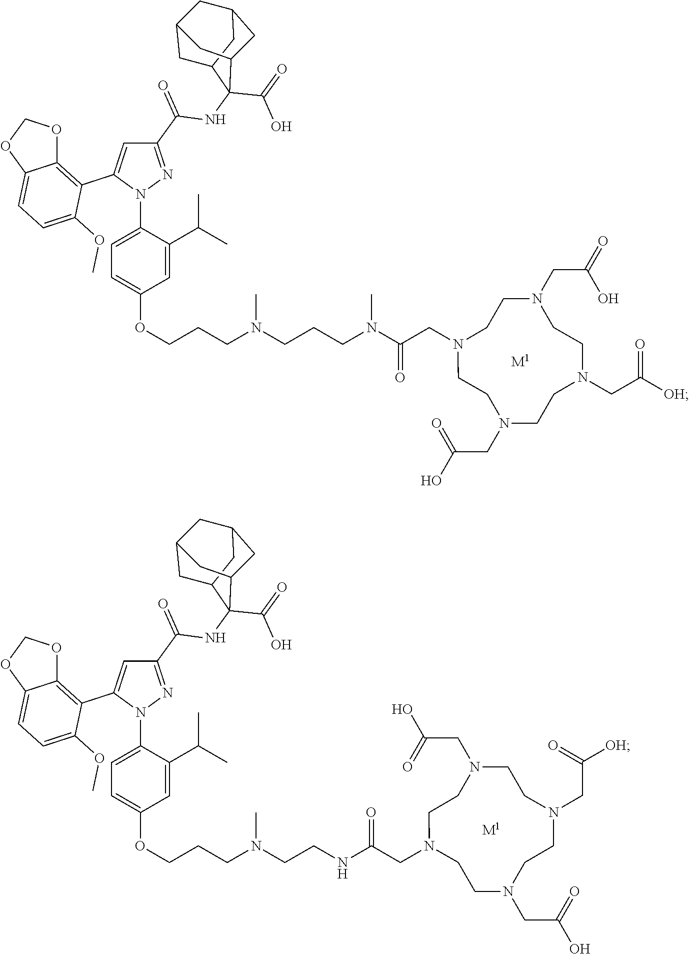

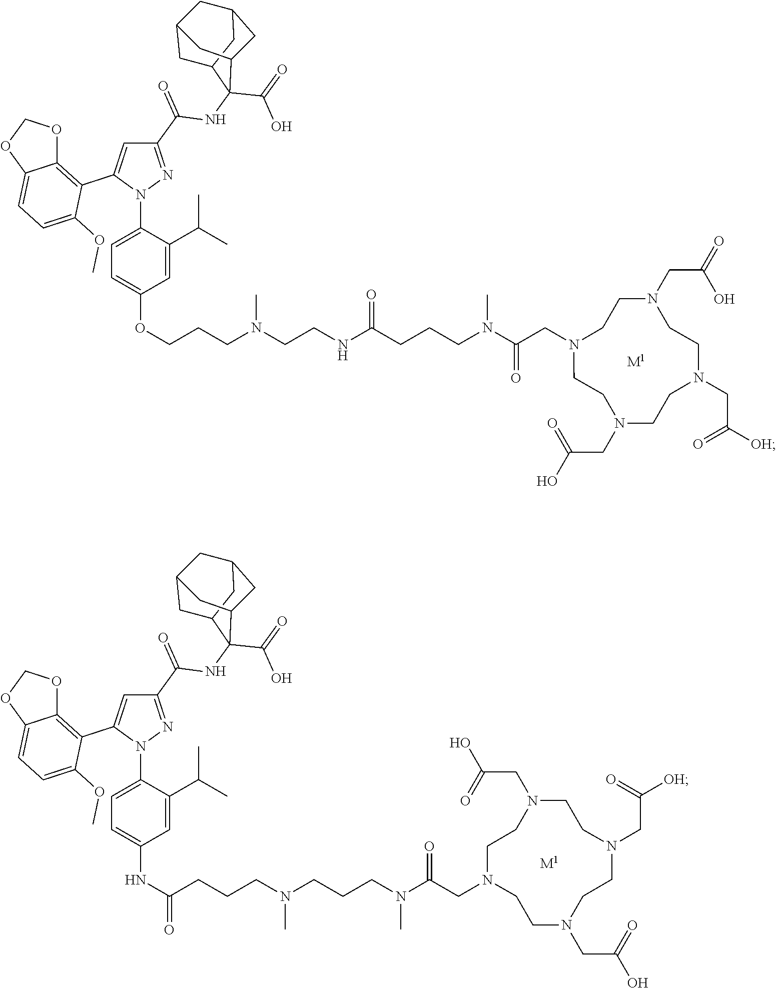

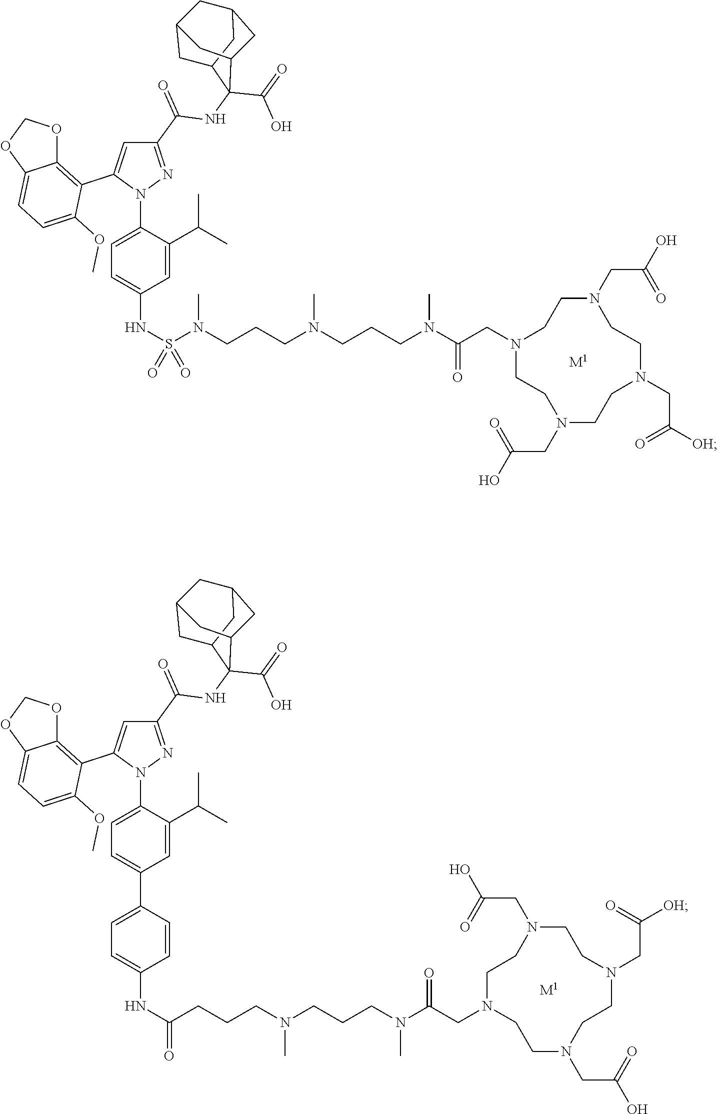

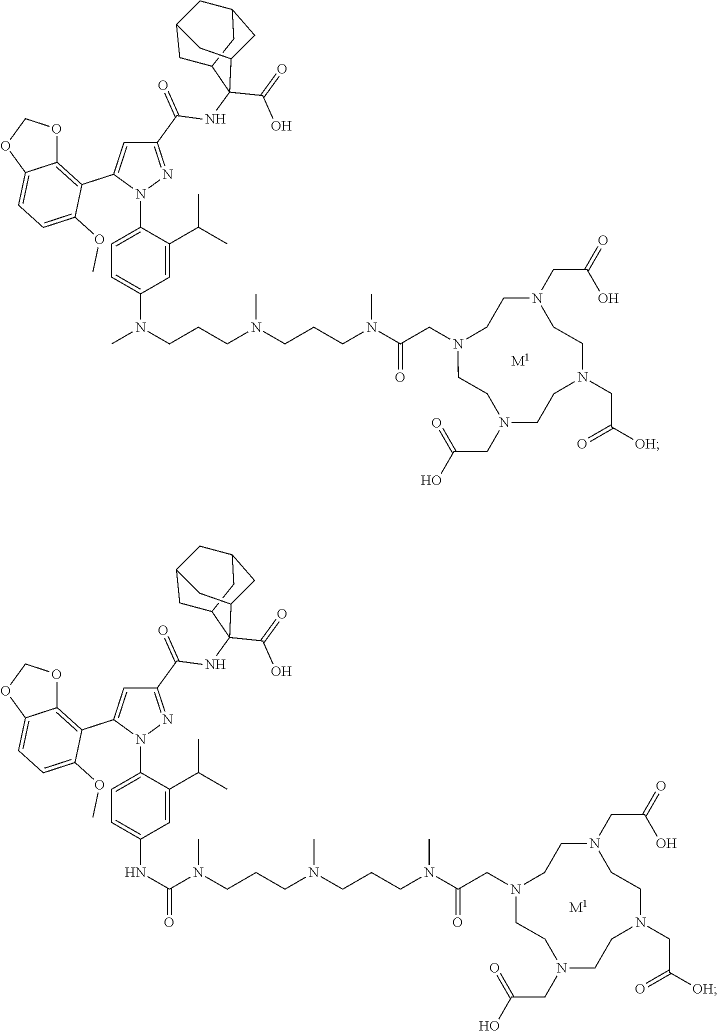

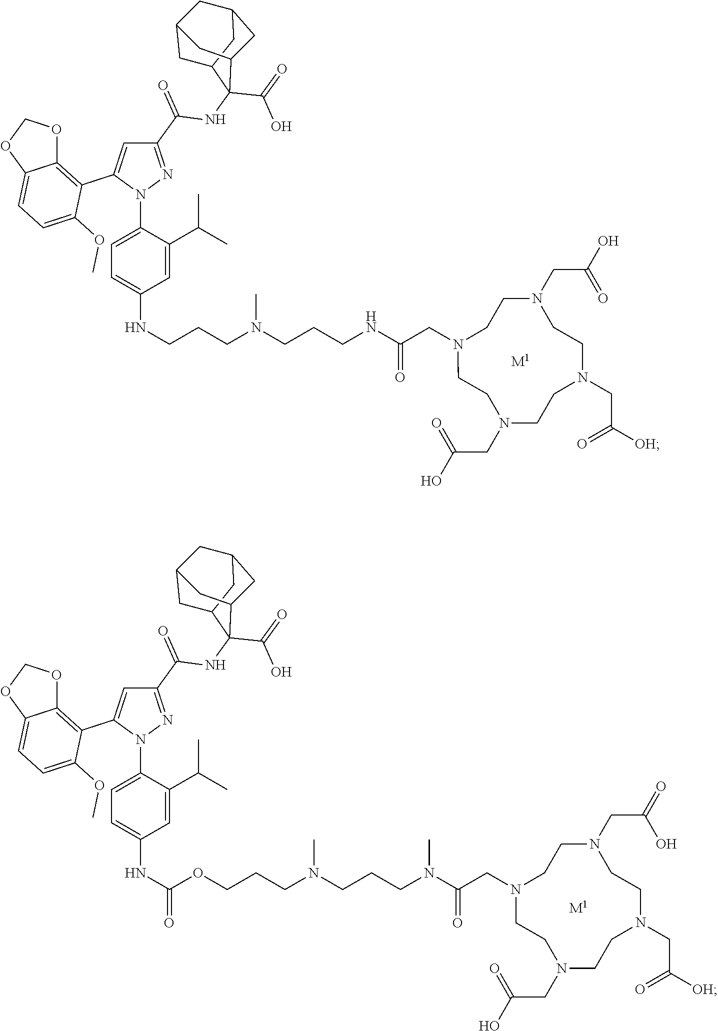

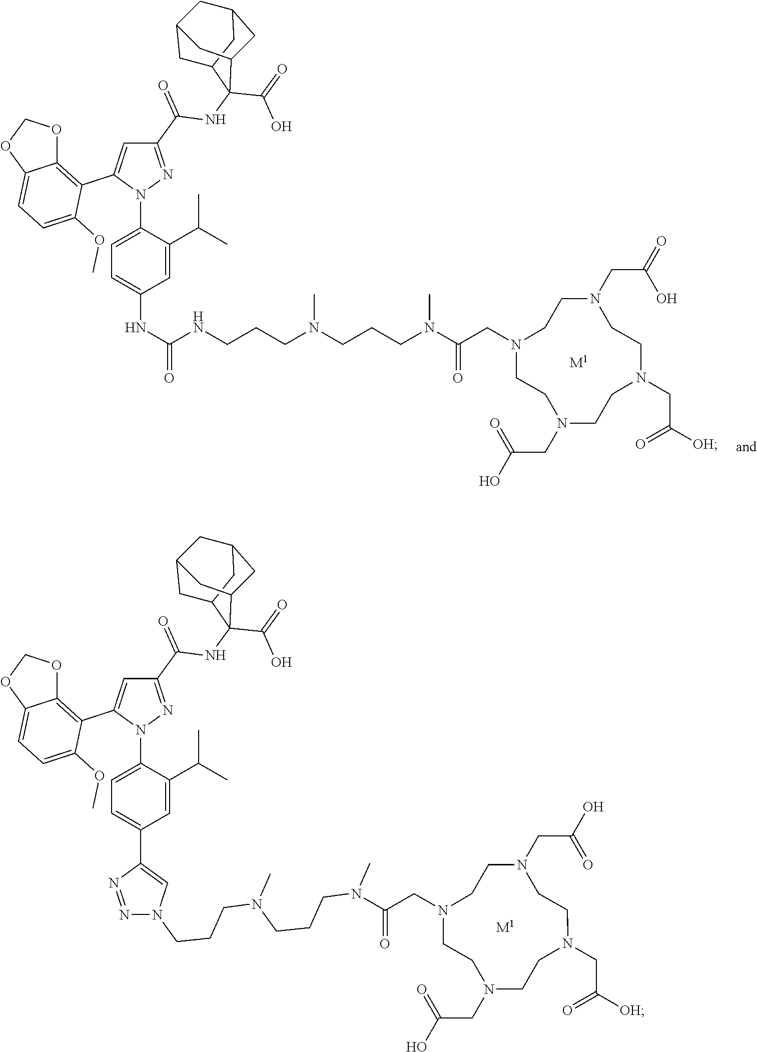

or a pharmaceutically acceptable salt thereof.

The present invention also provides a compound selected from:

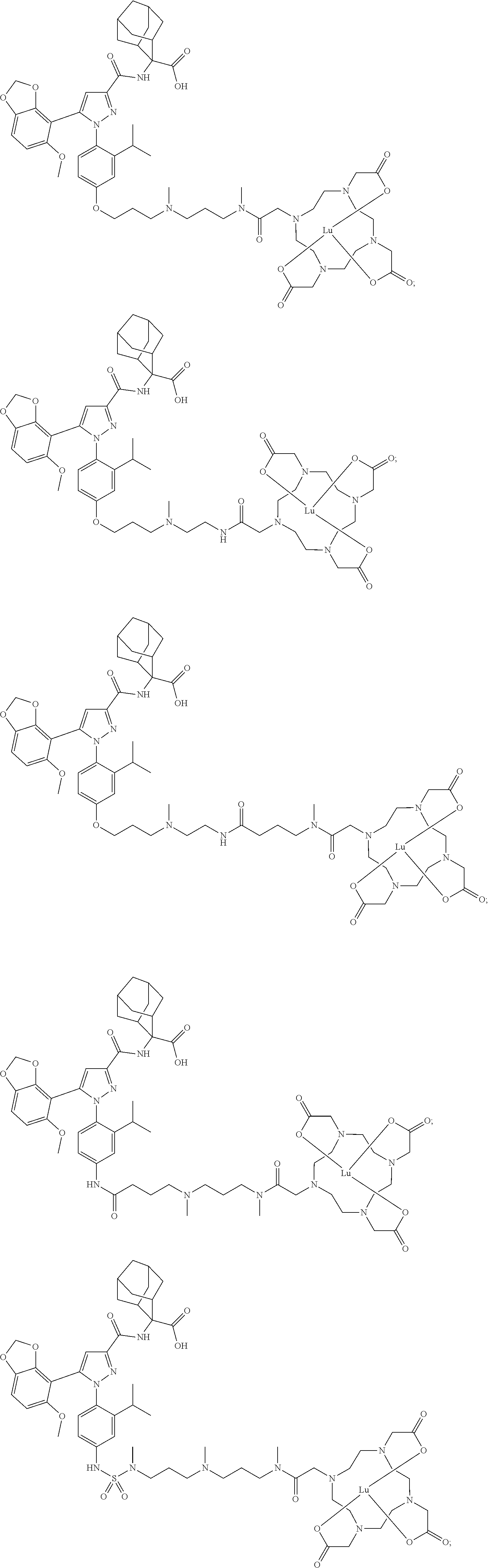

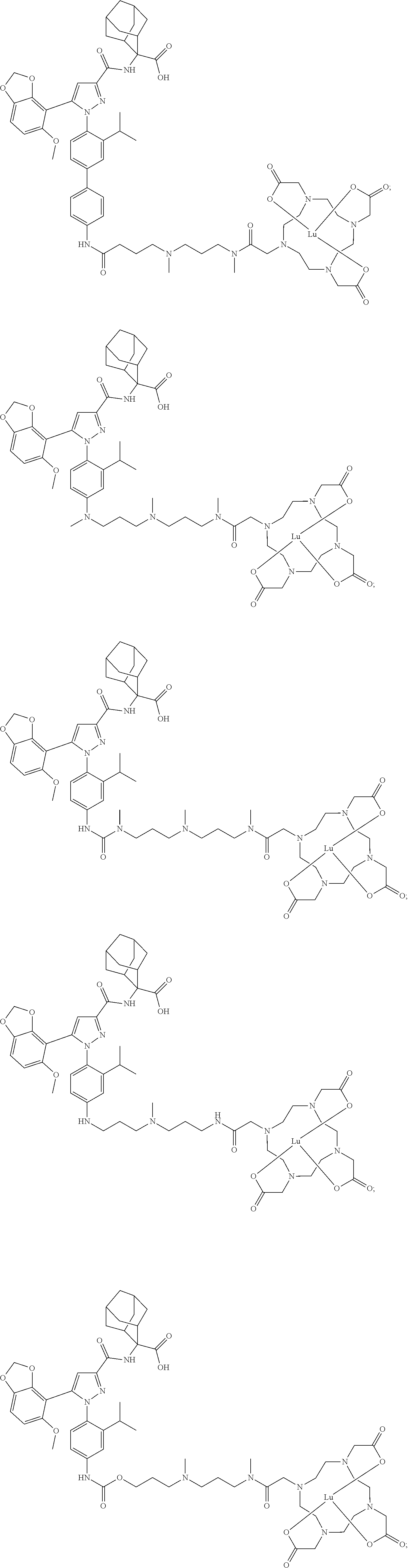

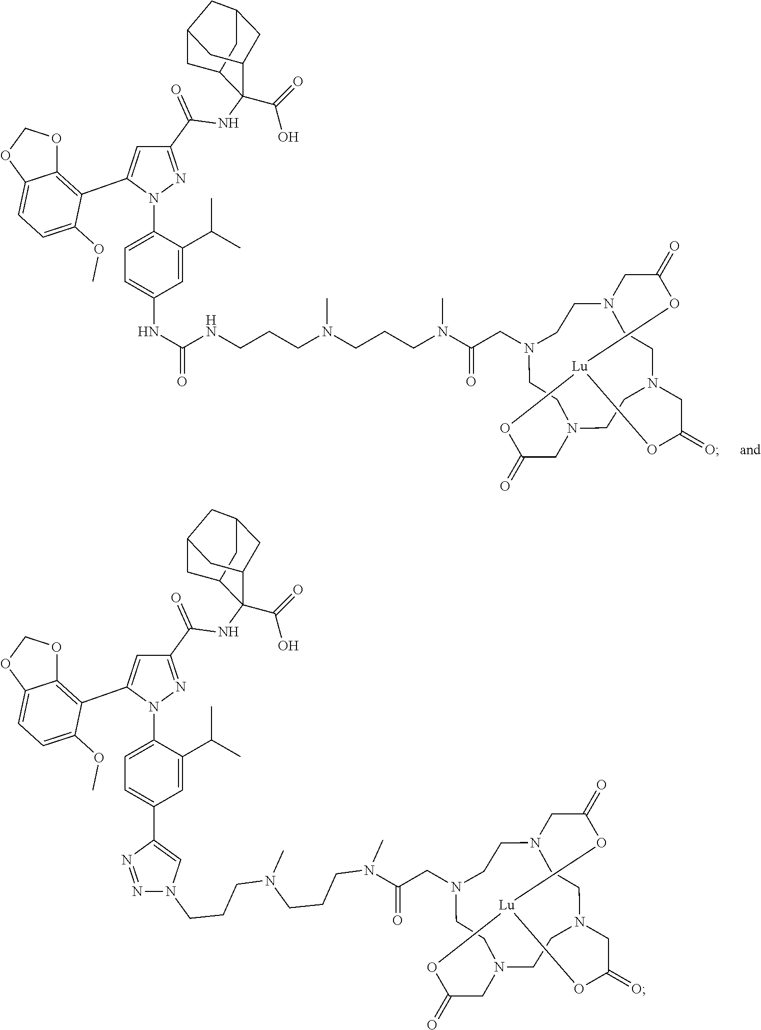

or a pharmaceutically acceptable salt thereof.

The present invention also provides a compound selected from:

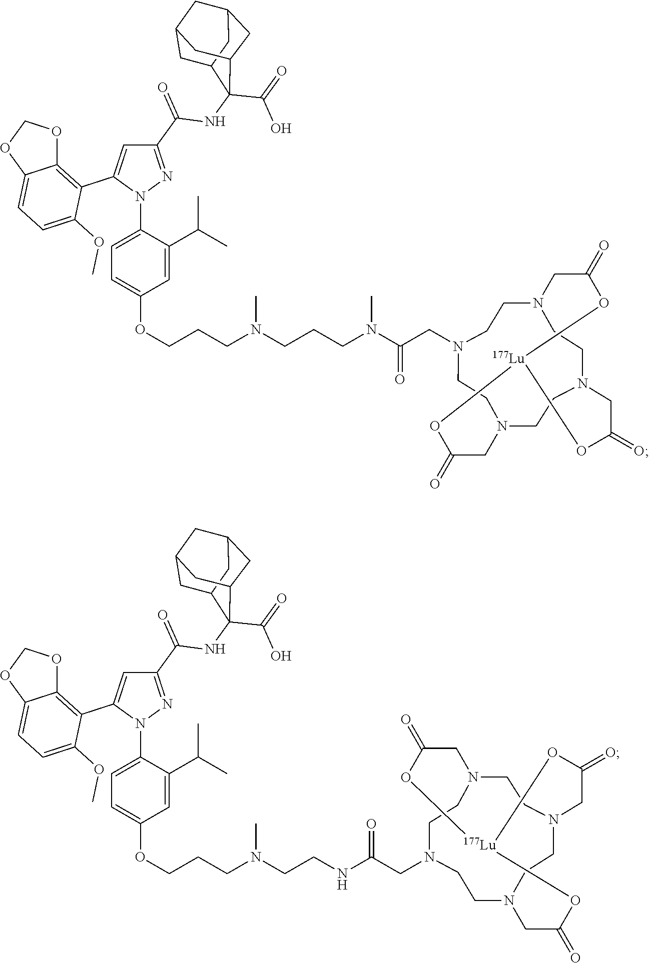

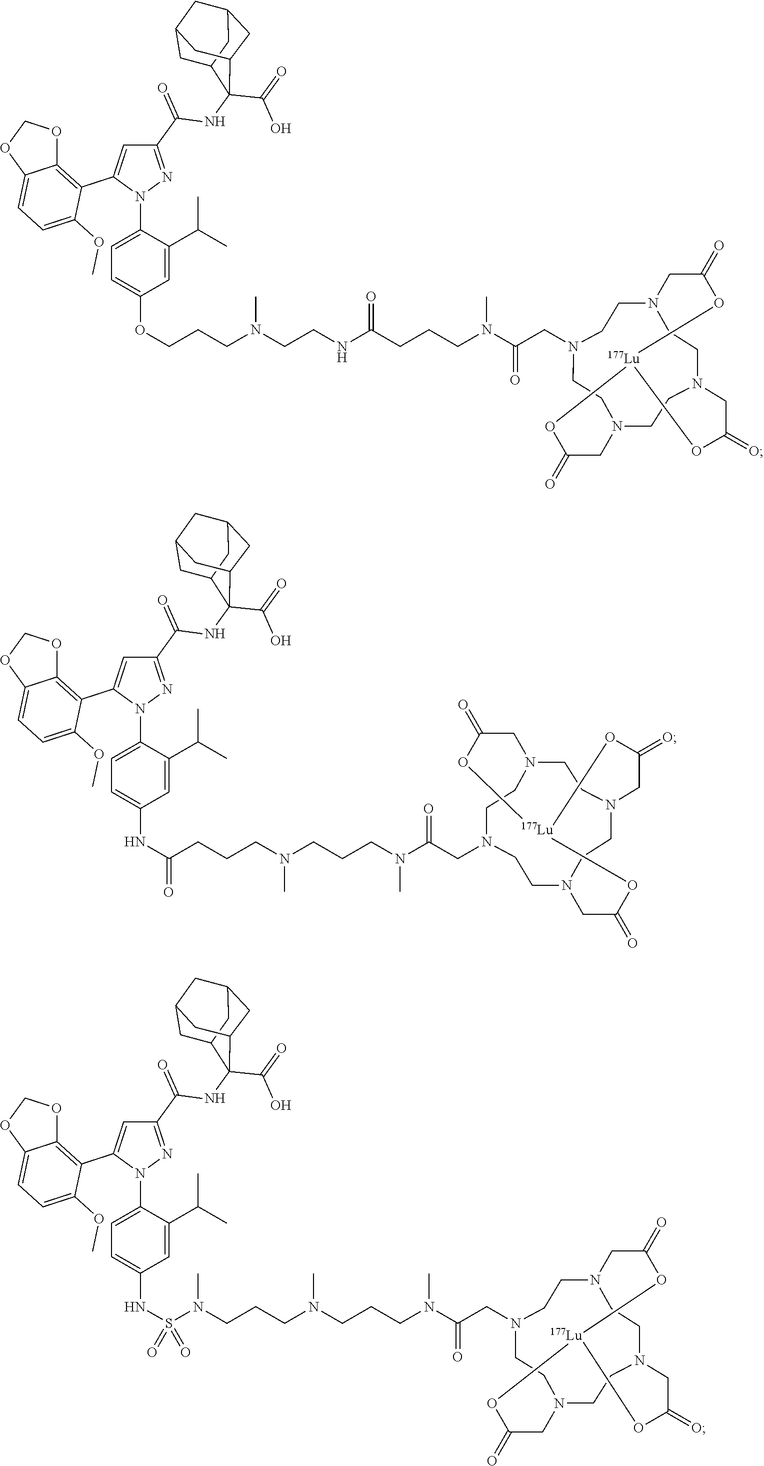

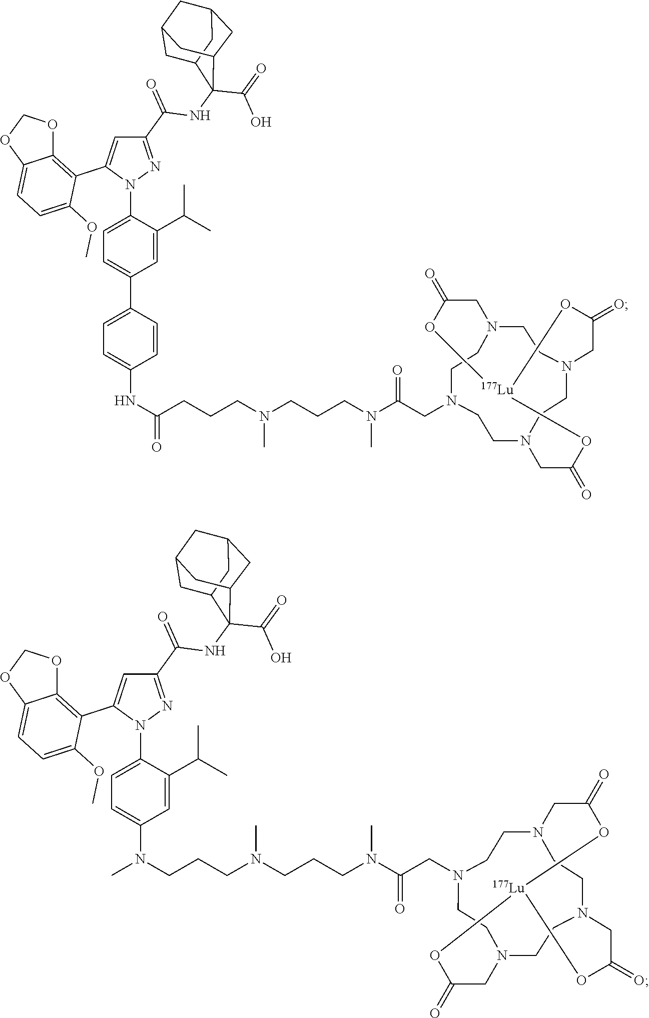

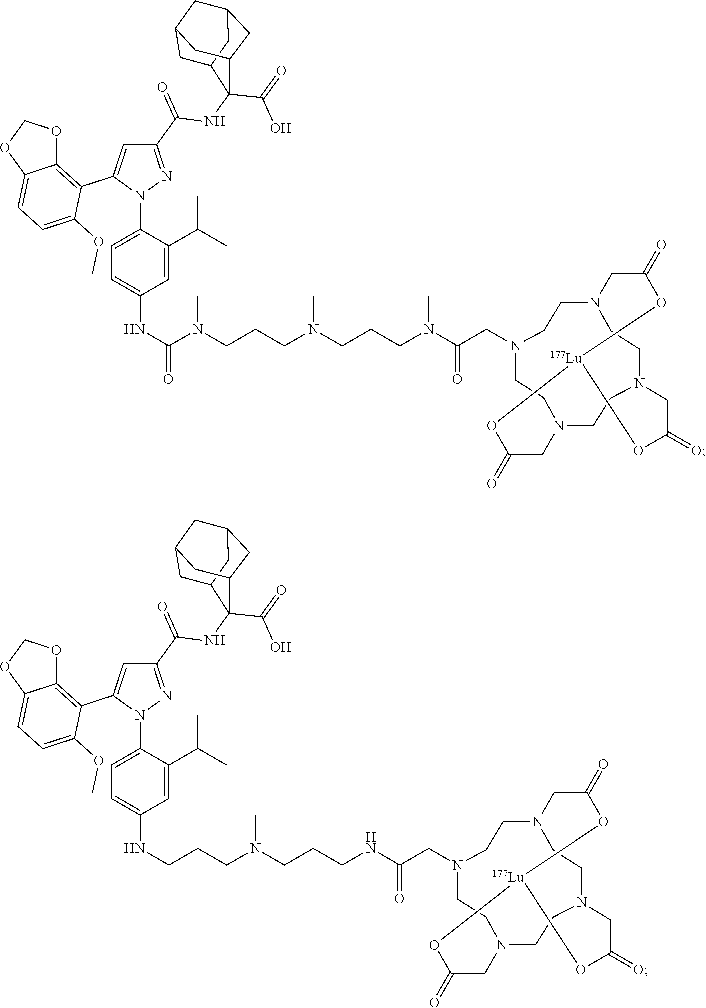

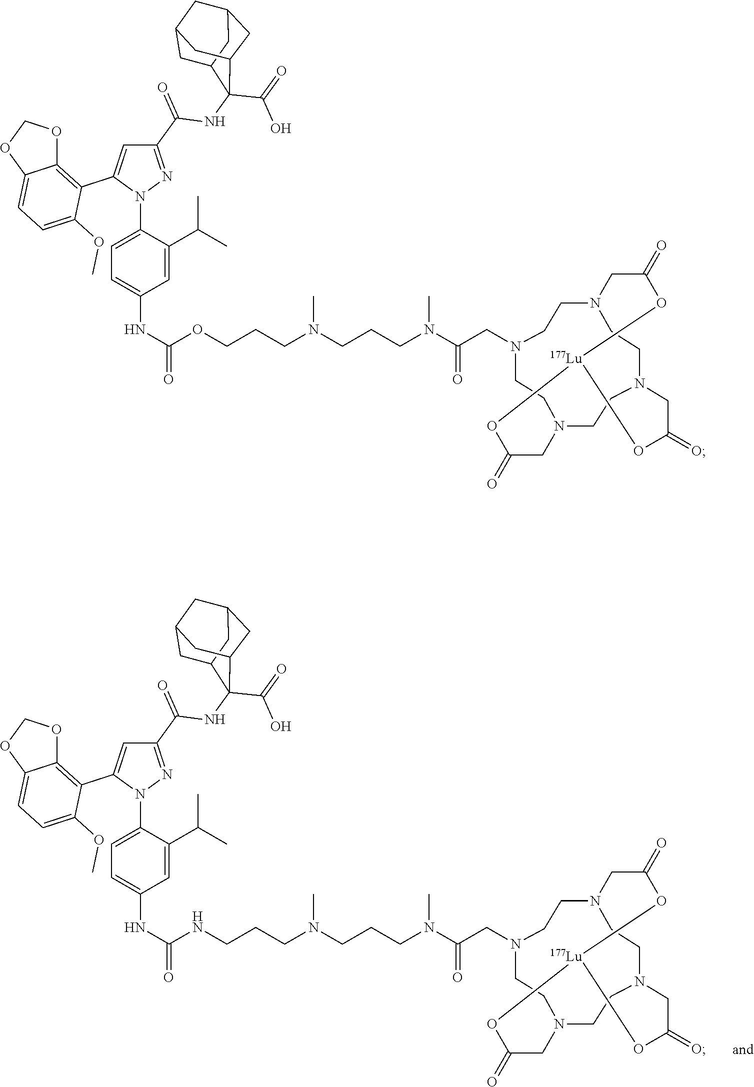

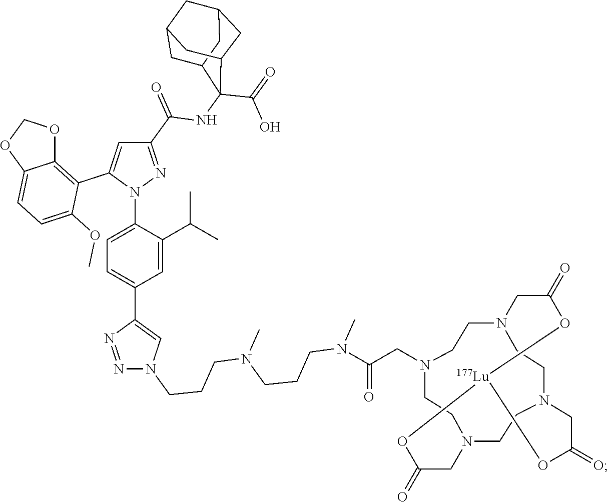

or a pharmaceutically acceptable salt thereof.

The present invention also provides a compound that is

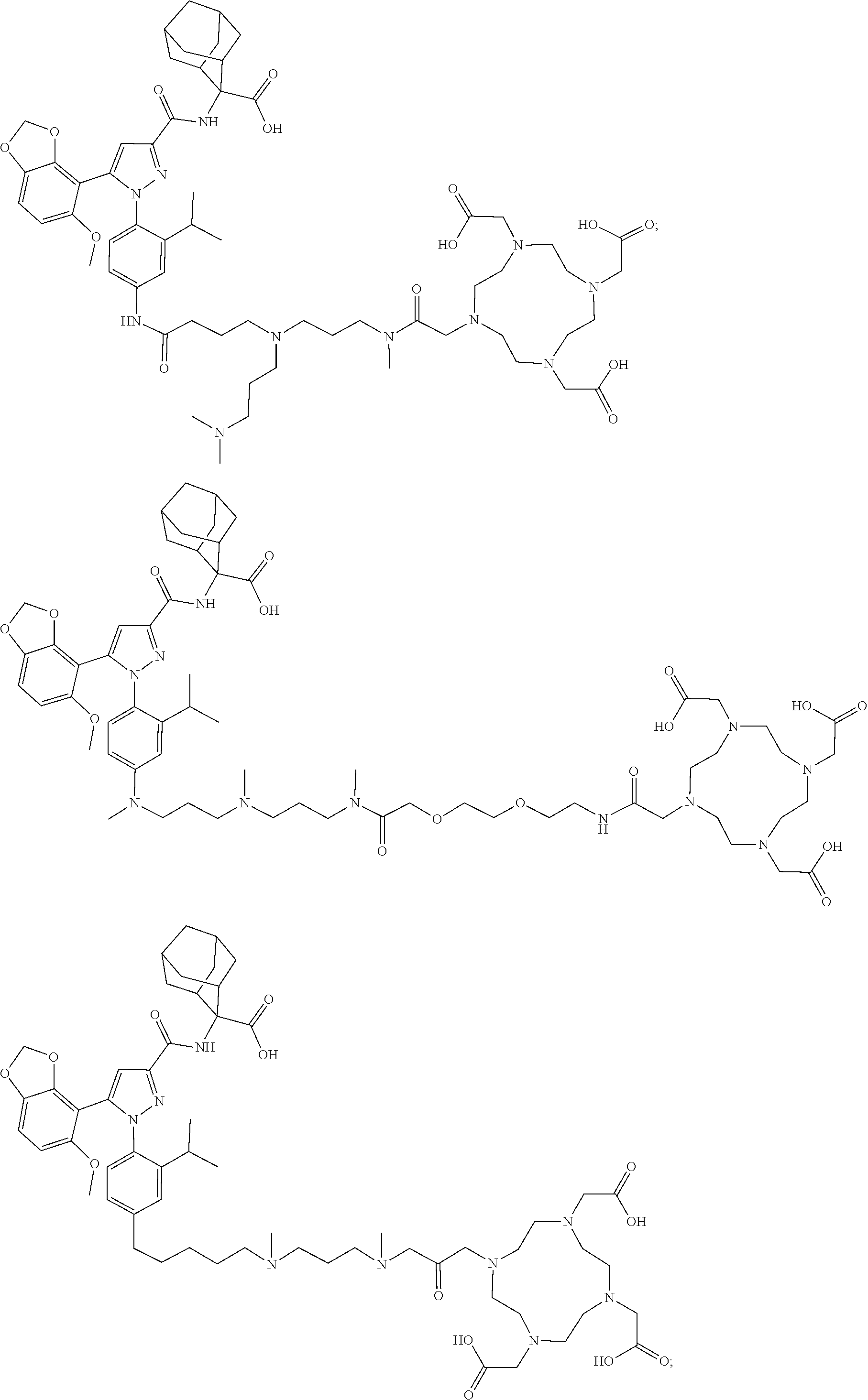

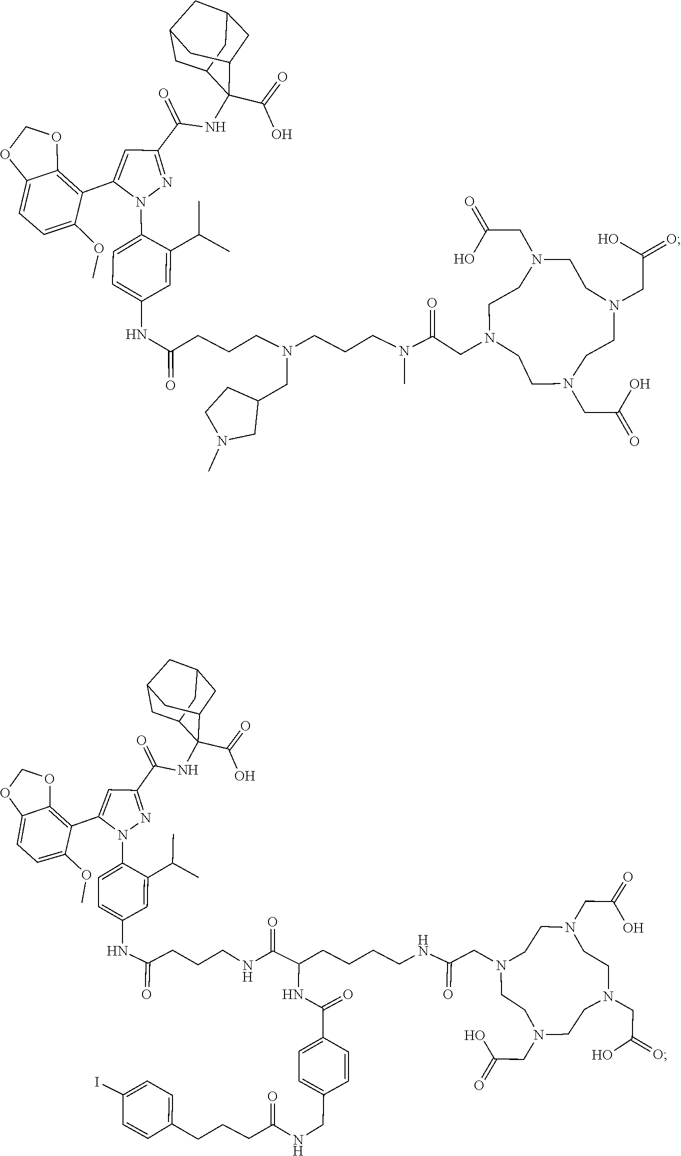 [SMILES: COc1ccc2c(c1-c1cc(C(=O)NC3(C(=O)O)C4CC5CC(C4)CC3C5)nn1-c1ccc(NC(=O)CCCN(CCCN(C)C(=O)CN3CCN(CC(=O)O)CCN(CC(=O)O)CCN(CC(=O)O)CC3)CC3CCN(C)C3)cc1C(C)C)OCO2 ; COc1ccc2c(c1-c1cc(C(=O)NC3(C(=O)O)C4CC5CC(C4)CC3C5)nn1-c1ccc(NC(=O)CCCNC(=O)C(CCCCNC(=O)CN3CCN(CC(=O)O)CCN(CC(=O)O)CCN(CC(=O)O)CC3)NC(=O)c3ccc(CNC(=O)CCCc4ccc(I)cc4)cc3)cc1C(C)C)OCO2]

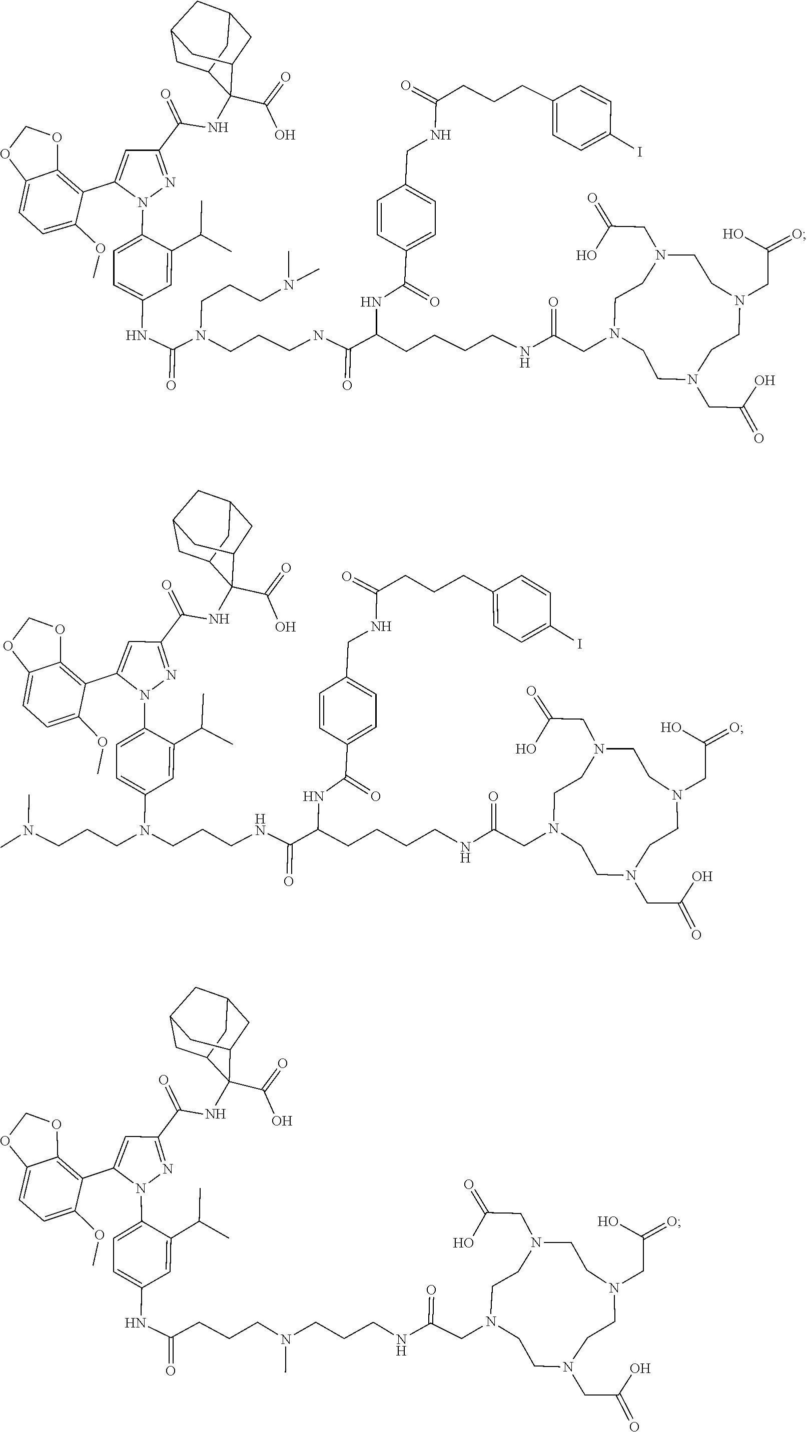

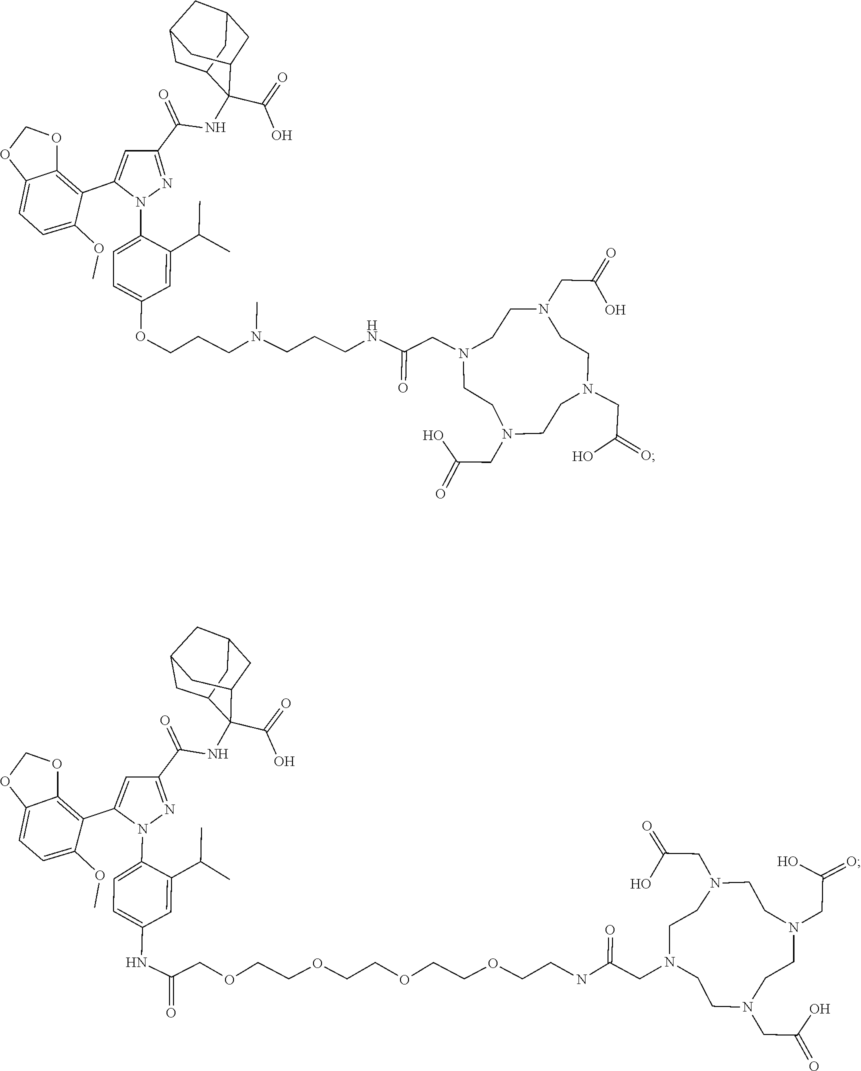

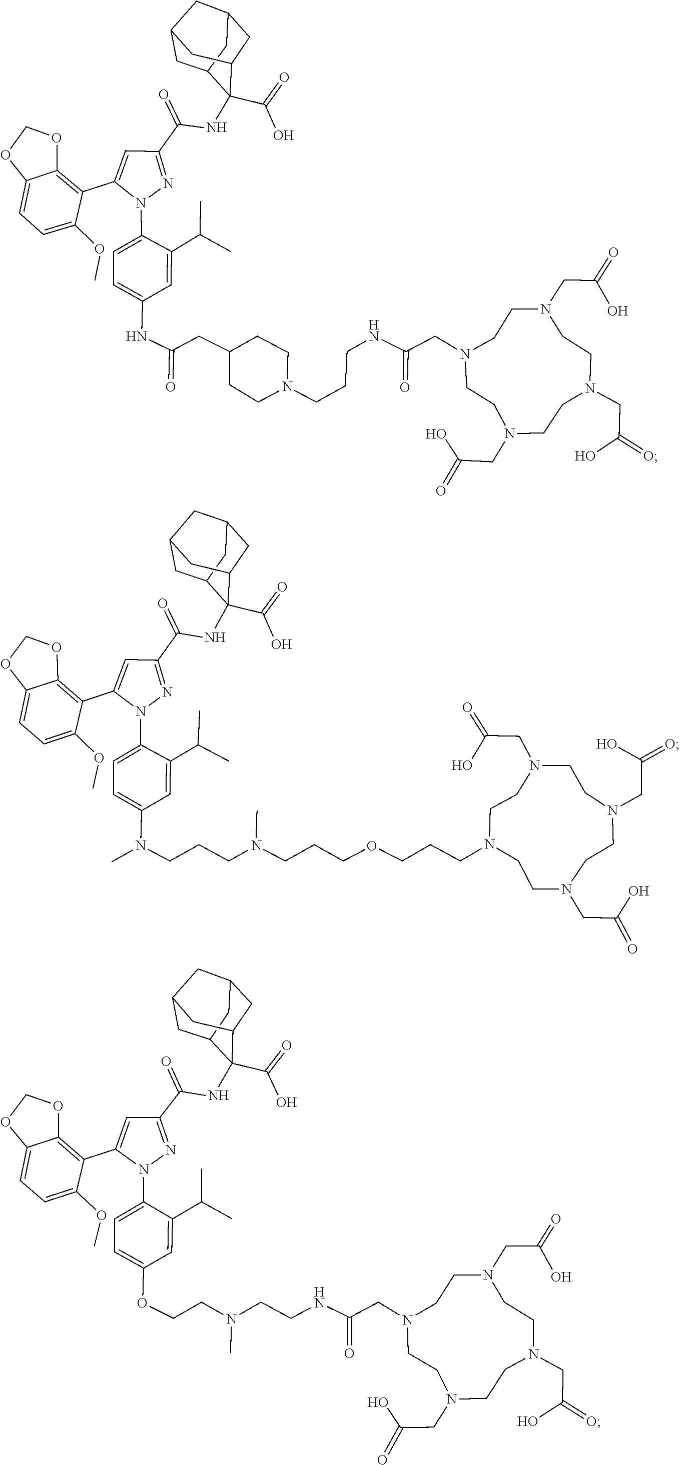 [SMILES: COc1ccc2c(c1-c1cc(C(=O)NC3(C(=O)O)C4CC5CC(C4)CC3C5)nn1-c1ccc(NC(=O)CC3CCN(CCCNC(=O)CN4CCN(CC(=O)O)CCN(CC(=O)O)CCN(CC(=O)O)CC4)CC3)cc1C(C)C)OCO2 ; COc1ccc2c(c1-c1cc(C(=O)NC3(C(=O)O)C4CC5CC(C4)CC3C5)nn1-c1ccc(N(C)CCCN(C)CCCOCCCN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(OCCN(C)CCNC(=O)CN3CCN(CC(=O)O)CCN(CC(=O)O)CCN(CC(=O)O)CC3)cc1C(C)C)OCO2]

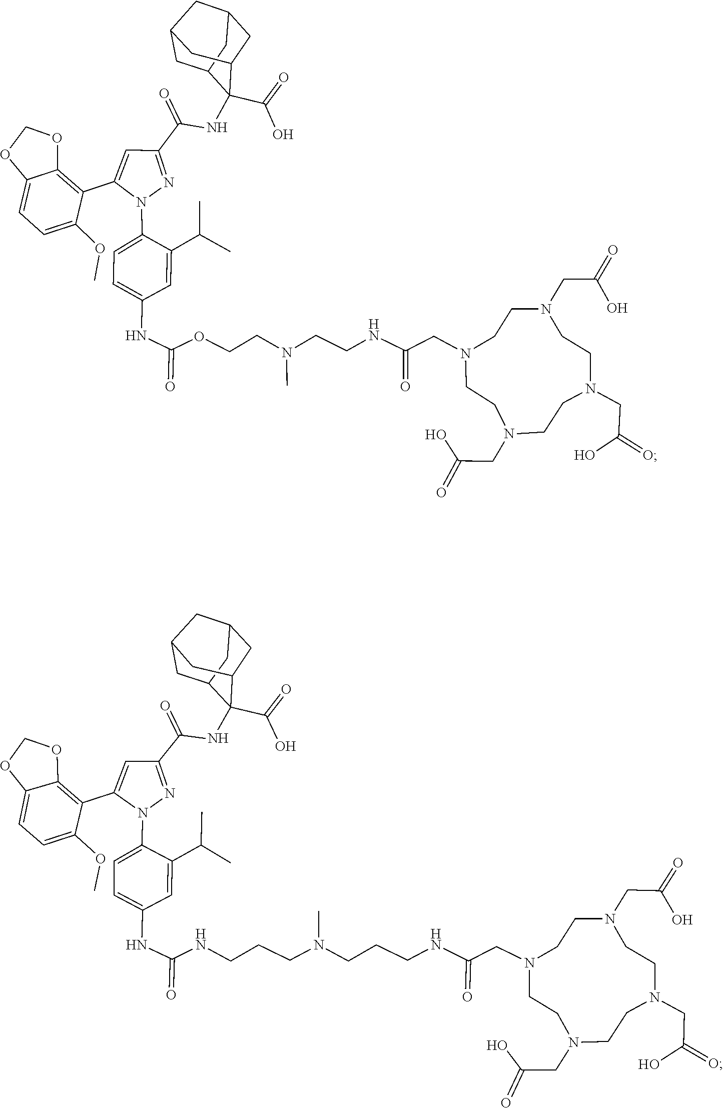 [SMILES: COc1ccc2c(c1-c1cc(C(=O)NC3(C(=O)O)C4CC5CC(C4)CC3C5)nn1-c1ccc(NC(=O)OCCN(C)CCNC(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(NC(=O)NCCCN(C)CCCNC(=O)CN3CCN(CC(=O)O)CCN(CC(=O)O)CCN(CC(=O)O)CC3)cc1C(C)C)OCO2]

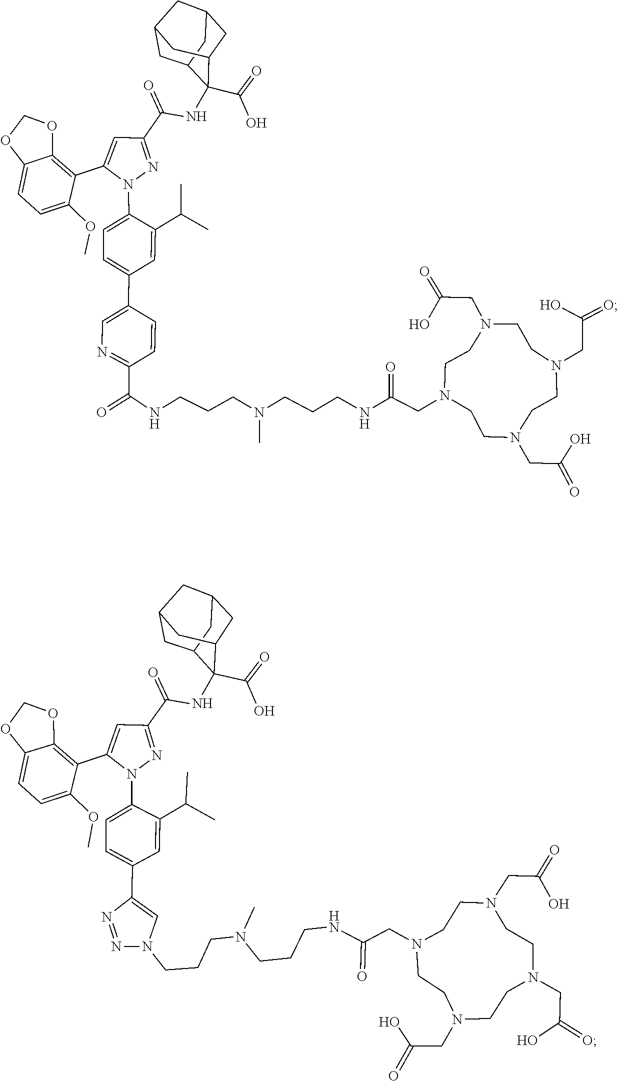 [SMILES: COc1ccc2c(c1-c1cc(C(=O)NC3(C(=O)O)C4CC5CC(C4)CC3C5)nn1-c1ccc(-c3ccc(C(=O)NCCCN(C)CCCNC(=O)CN4CCN(CC(=O)O)CCN(CC(=O)O)CCN(CC(=O)O)CC4)nc3)cc1C(C)C)OCO2 ; COc1ccc2c(c1-c1cc(C(=O)NC3(C(=O)O)C4CC5CC(C4)CC3C5)nn1-c1ccc(-c3cn(CCCN(C)CCCNC(=O)CN4CCN(CC(=O)O)CCN(CC(=O)O)CCN(CC(=O)O)CC4)nn3)cc1C(C)C)OCO2]

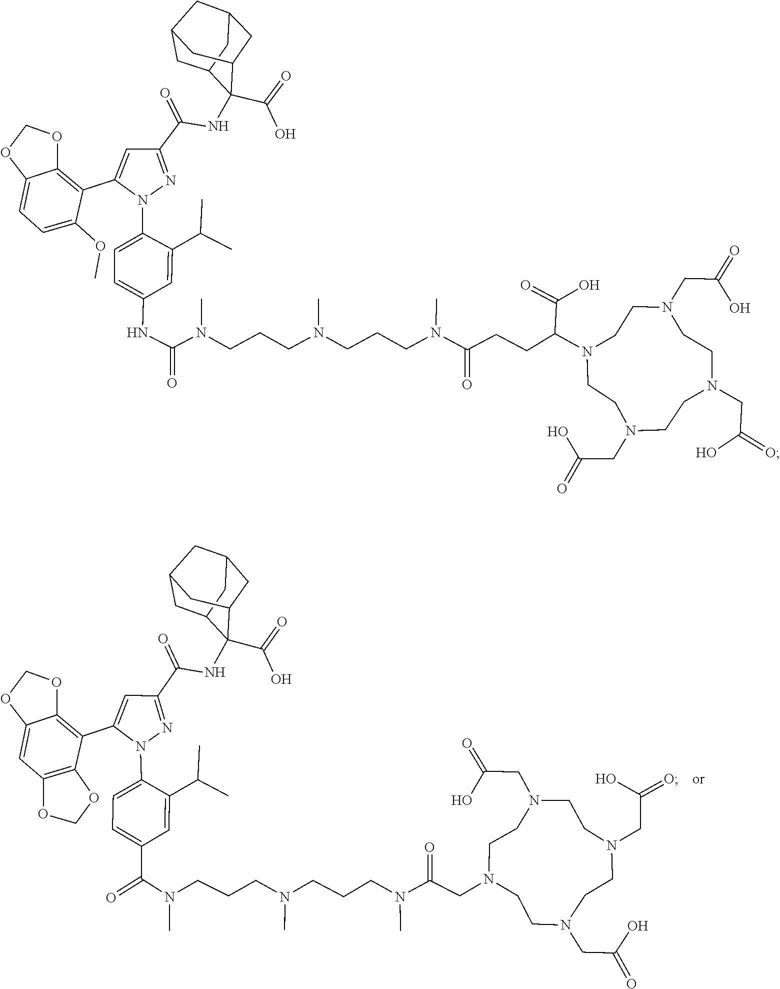 [SMILES: COc1ccc2c(c1-c1cc(C(=O)NC3(C(=O)O)C4CC5CC(C4)CC3C5)nn1-c1ccc(NC(=O)N(C)CCCN(C)CCCN(C)C(=O)CCC(C(=O)O)N3CCN(CC(=O)O)CCN(CC(=O)O)CCN(CC(=O)O)CC3)cc1C(C)C)OCO2 ; CC(C)c1cc(C(=O)N(C)CCCN(C)CCCN(C)C(=O)CN2CCN(CC(=O)O)CCN(CC(=O)O)CCN(CC(=O)O)CC2)ccc1-n1nc(C(=O)NC2(C(=O)O)C3CC4CC(C3)CC2C4)cc1-c1c2c(cc3c1OCO3)OCO2]

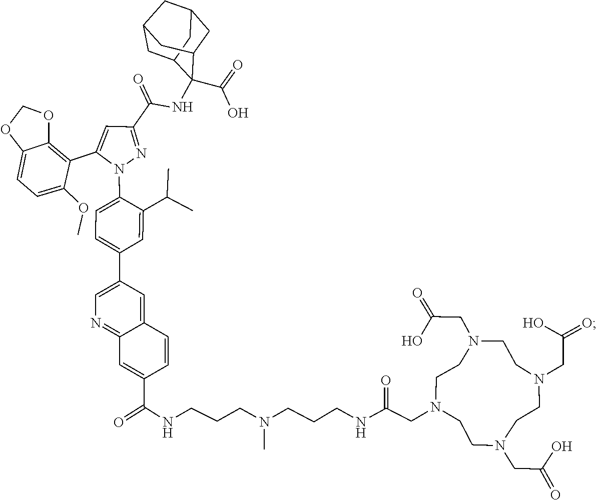 [SMILES: COc1ccc2c(c1-c1cc(C(=O)NC3(C(=O)O)C4CC5CC(C4)CC3C5)nn1-c1ccc(-c3cnc4cc(C(=O)NCCCN(C)CCCNC(=O)CN5CCN(CC(=O)O)CCN(CC(=O)O)CCN(CC(=O)O)CC5)ccc4c3)cc1C(C)C)OCO2]

or a pharmaceutically acceptable salt thereof.

The present invention also provides a compound that is

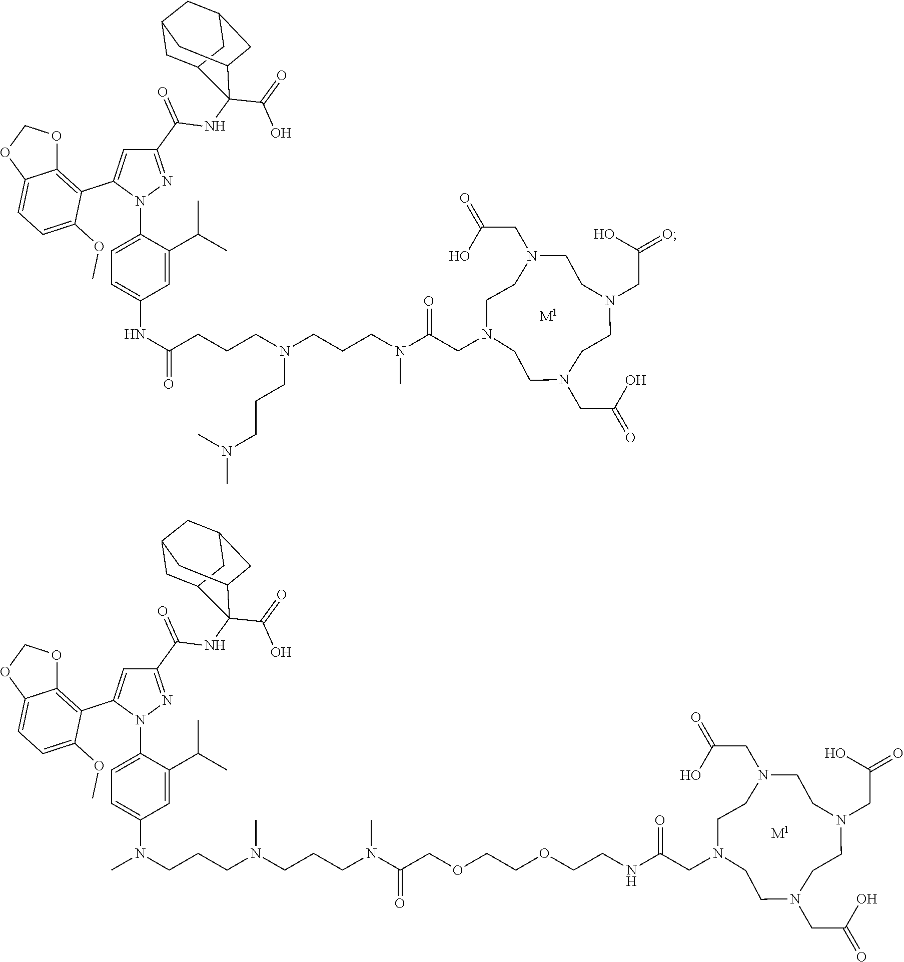

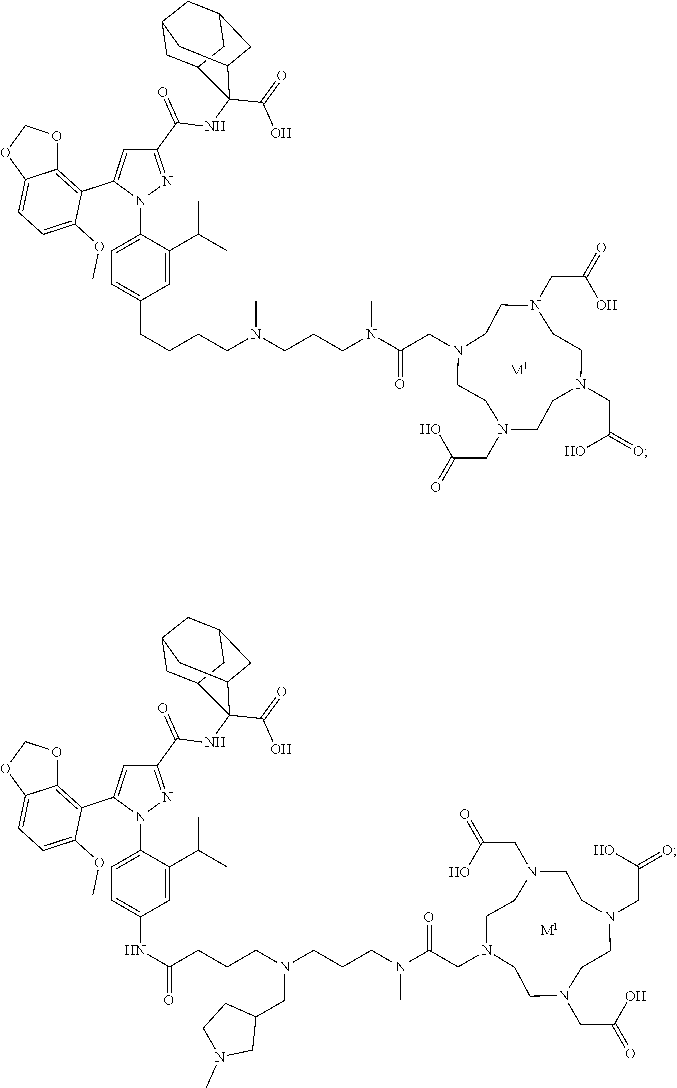

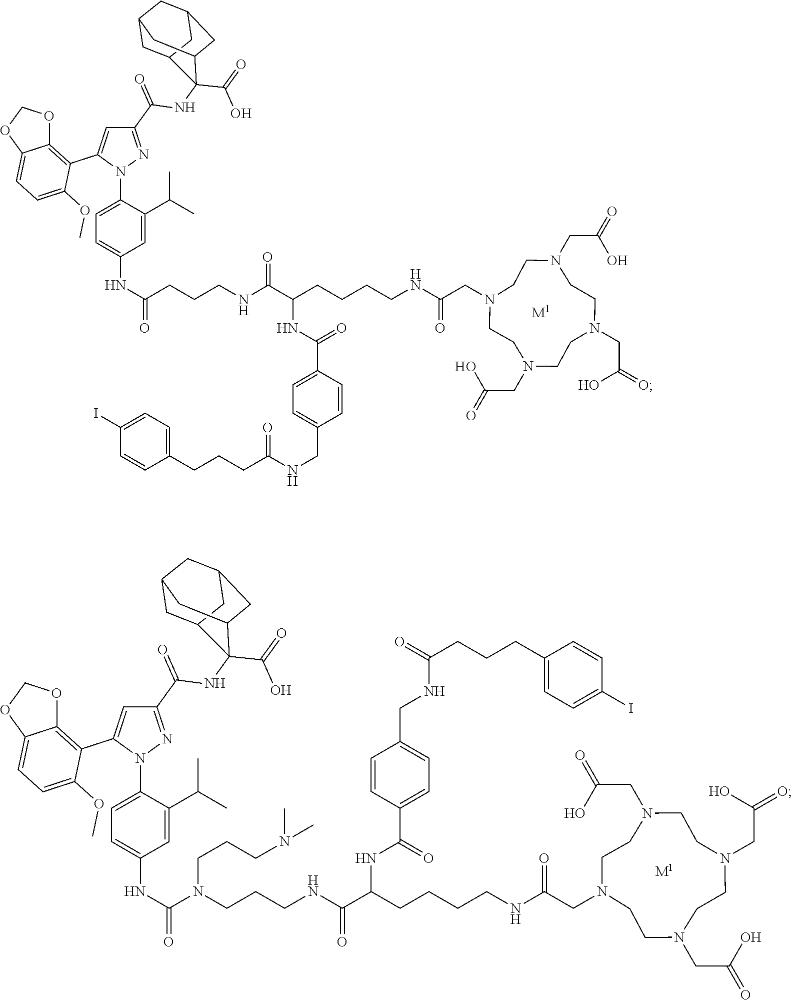

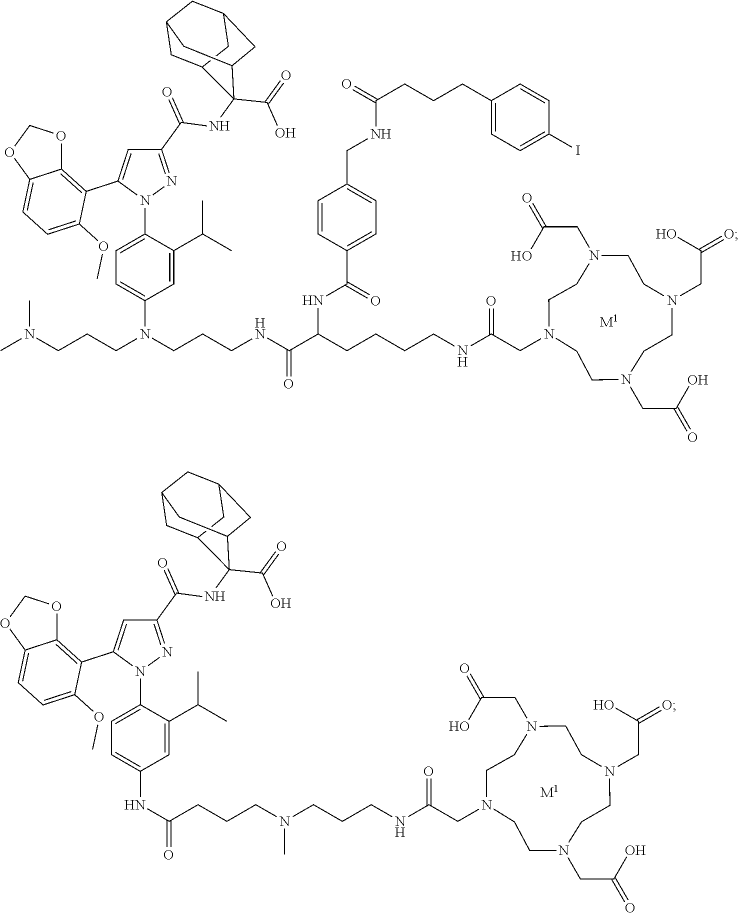

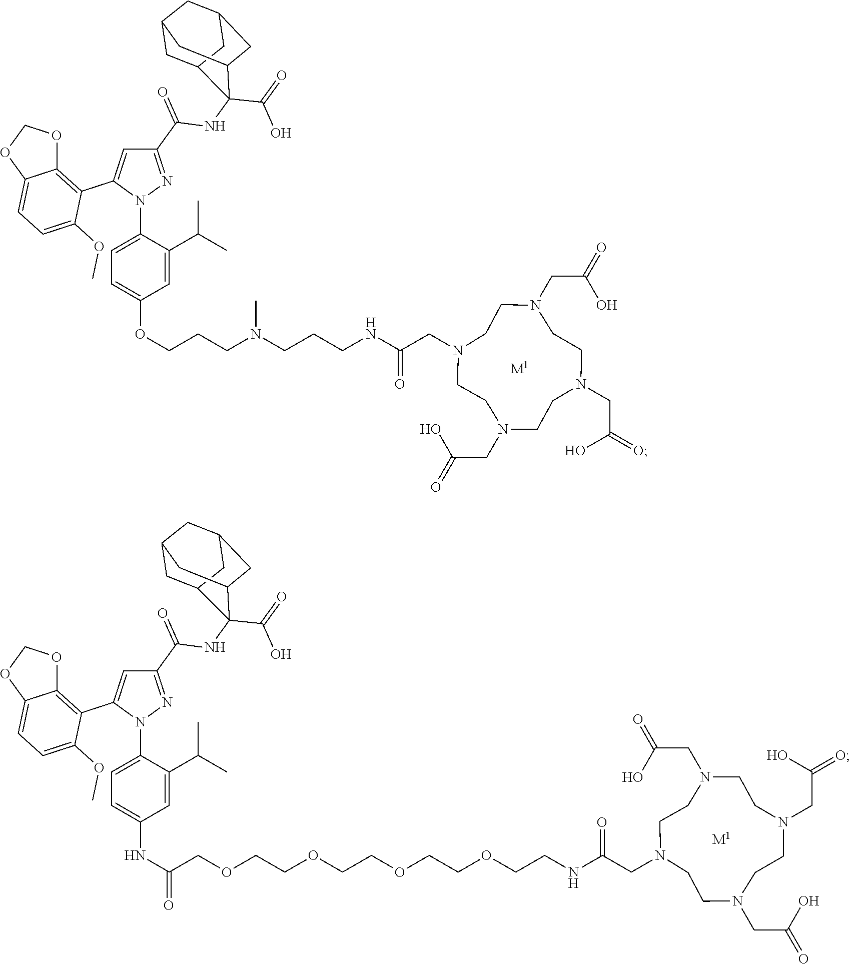

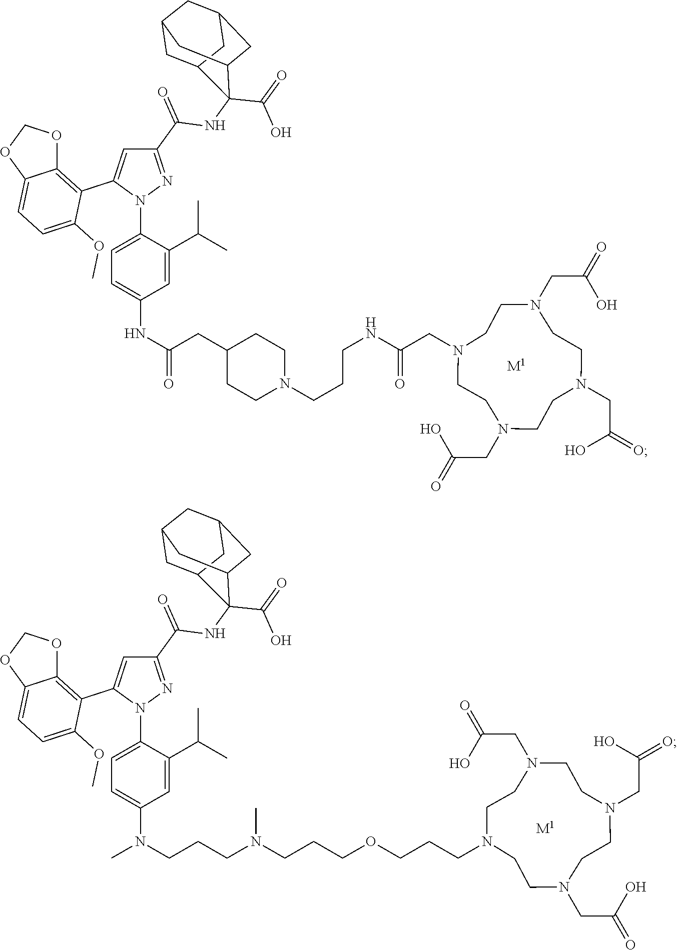

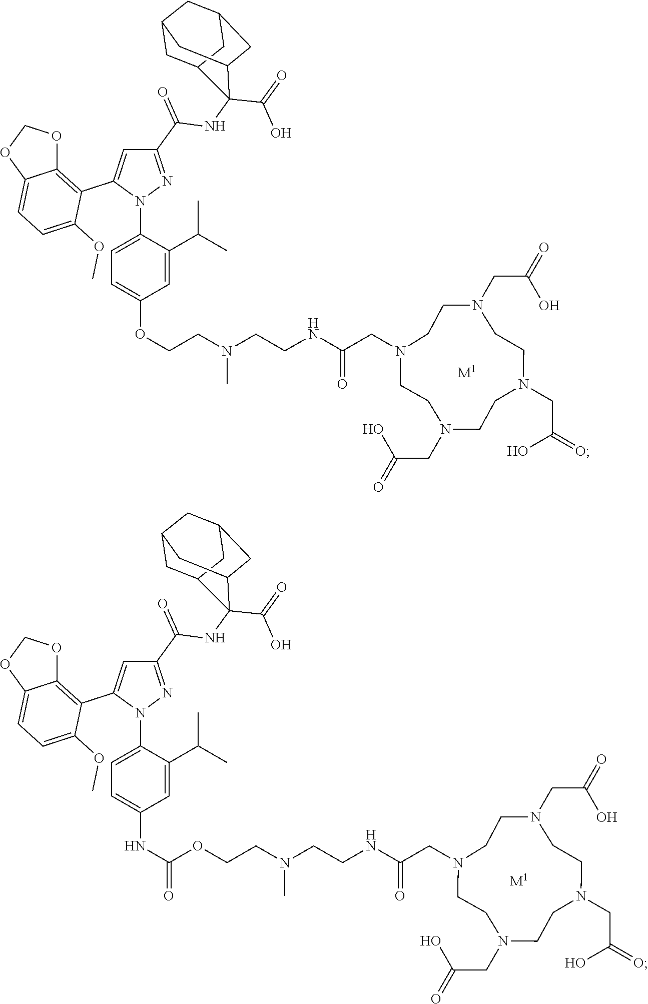

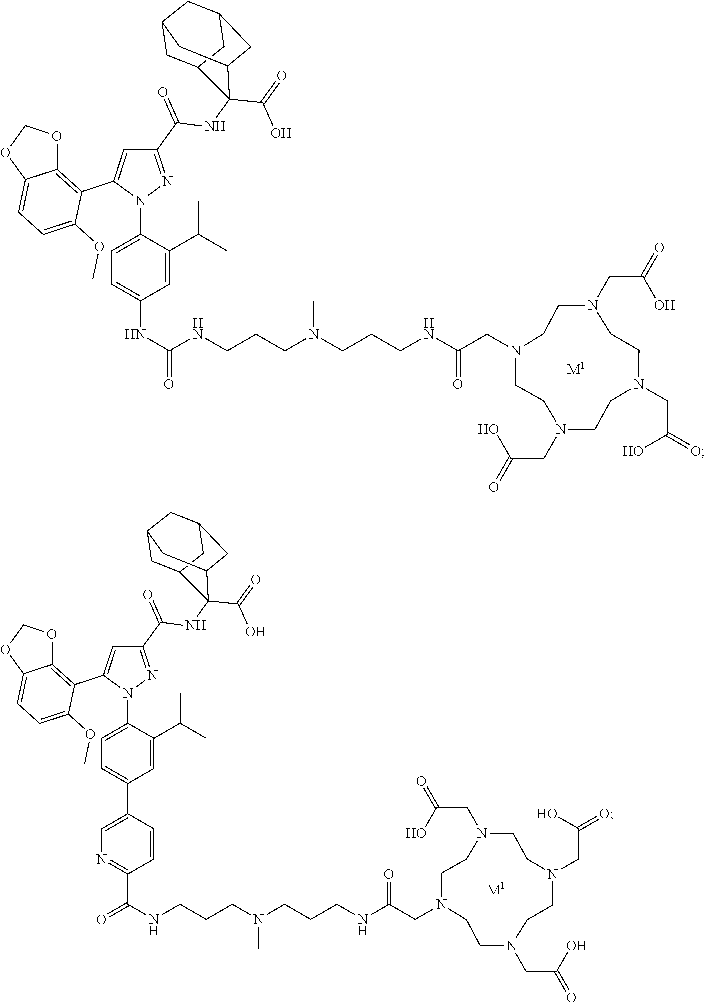

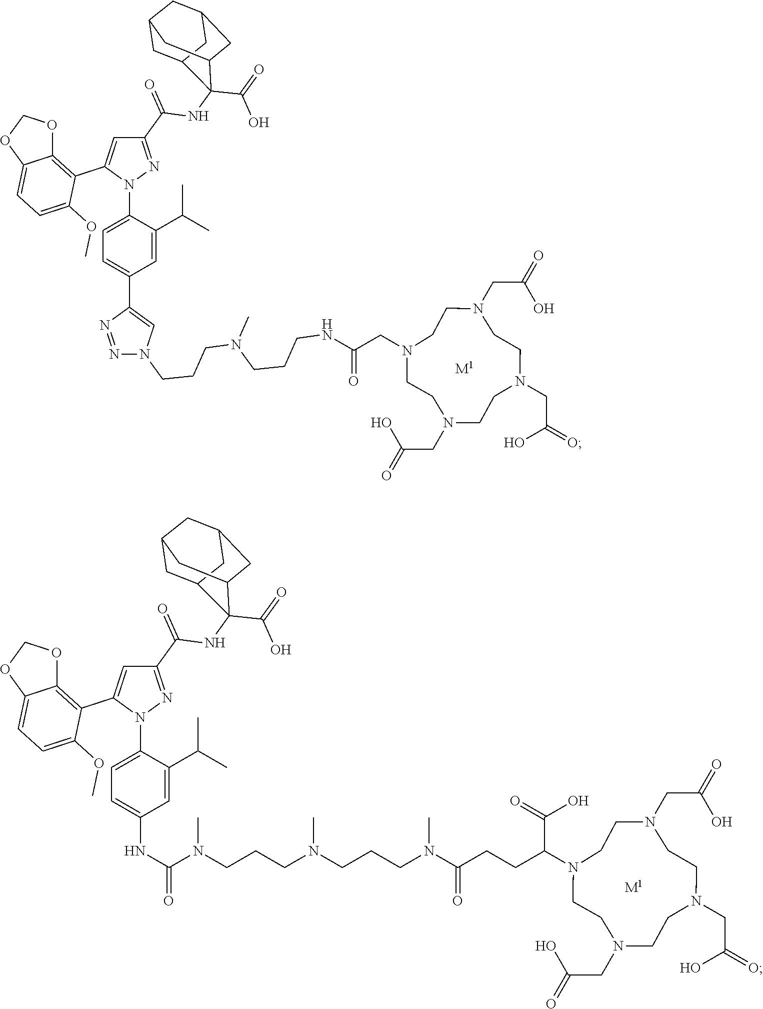

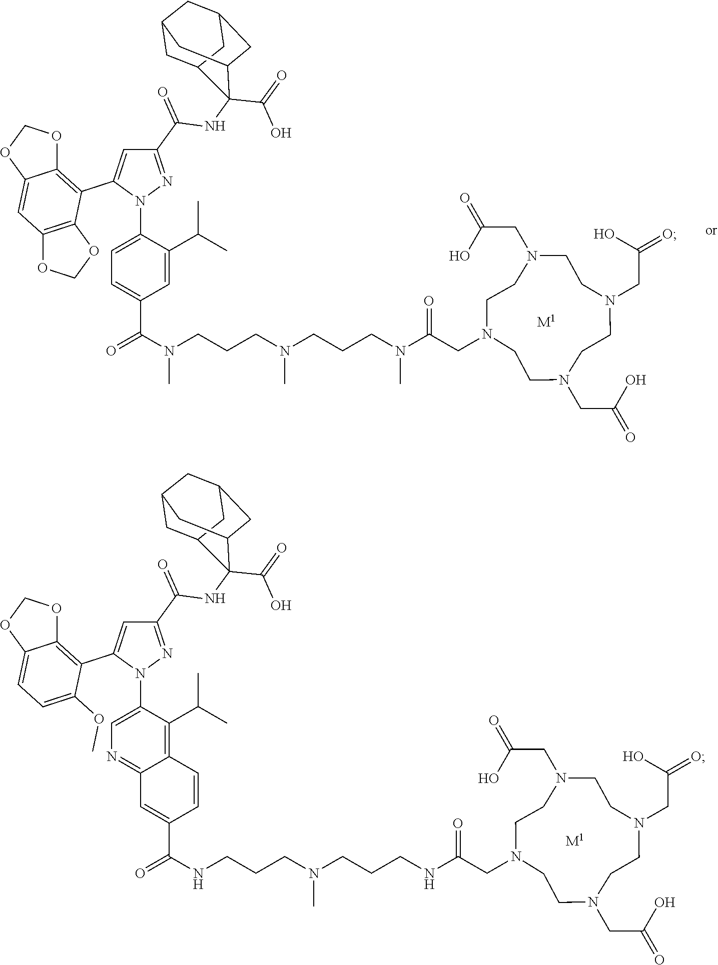

or a pharmaceutically acceptable salt thereof.

The present invention also provides a compound that is

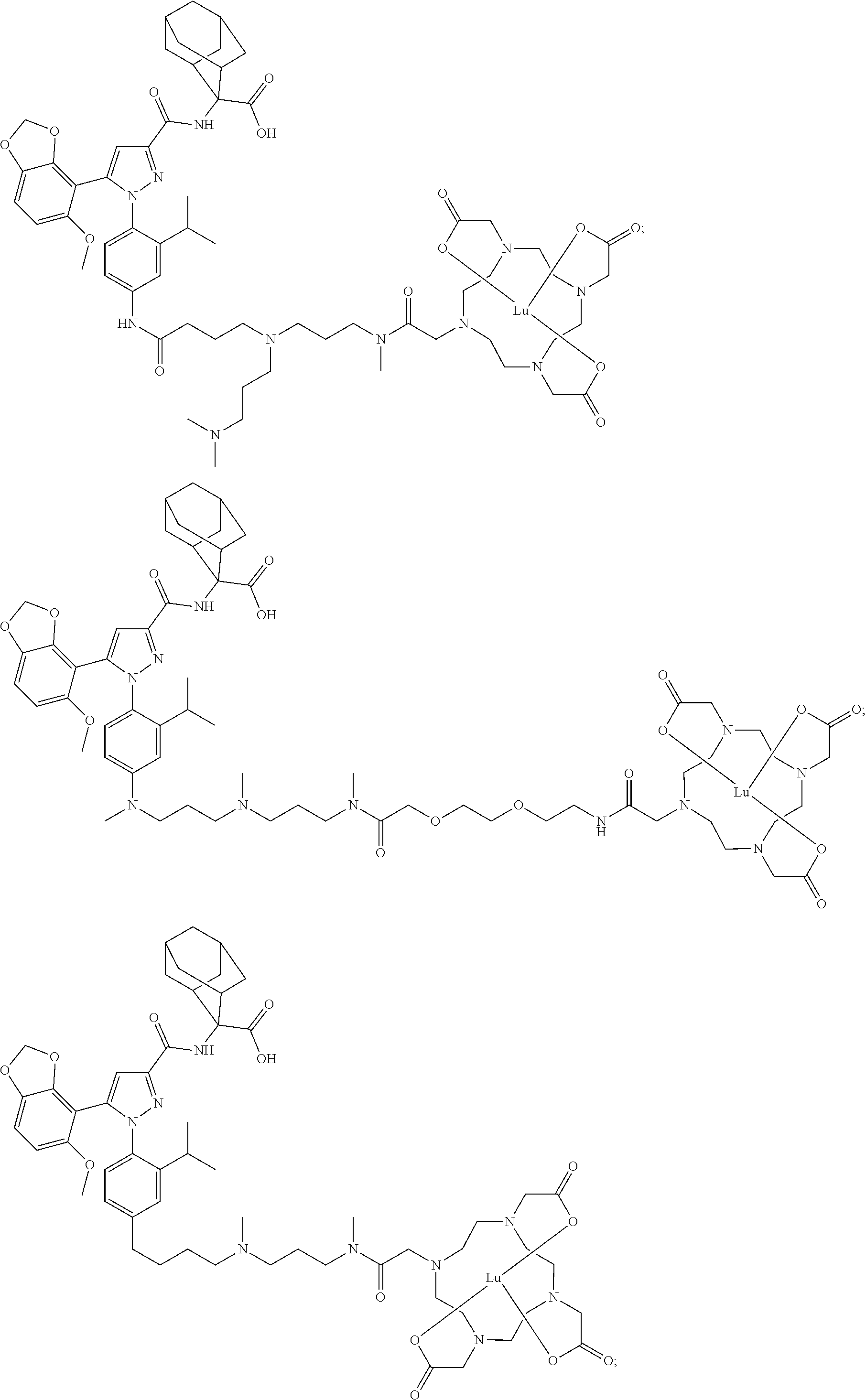

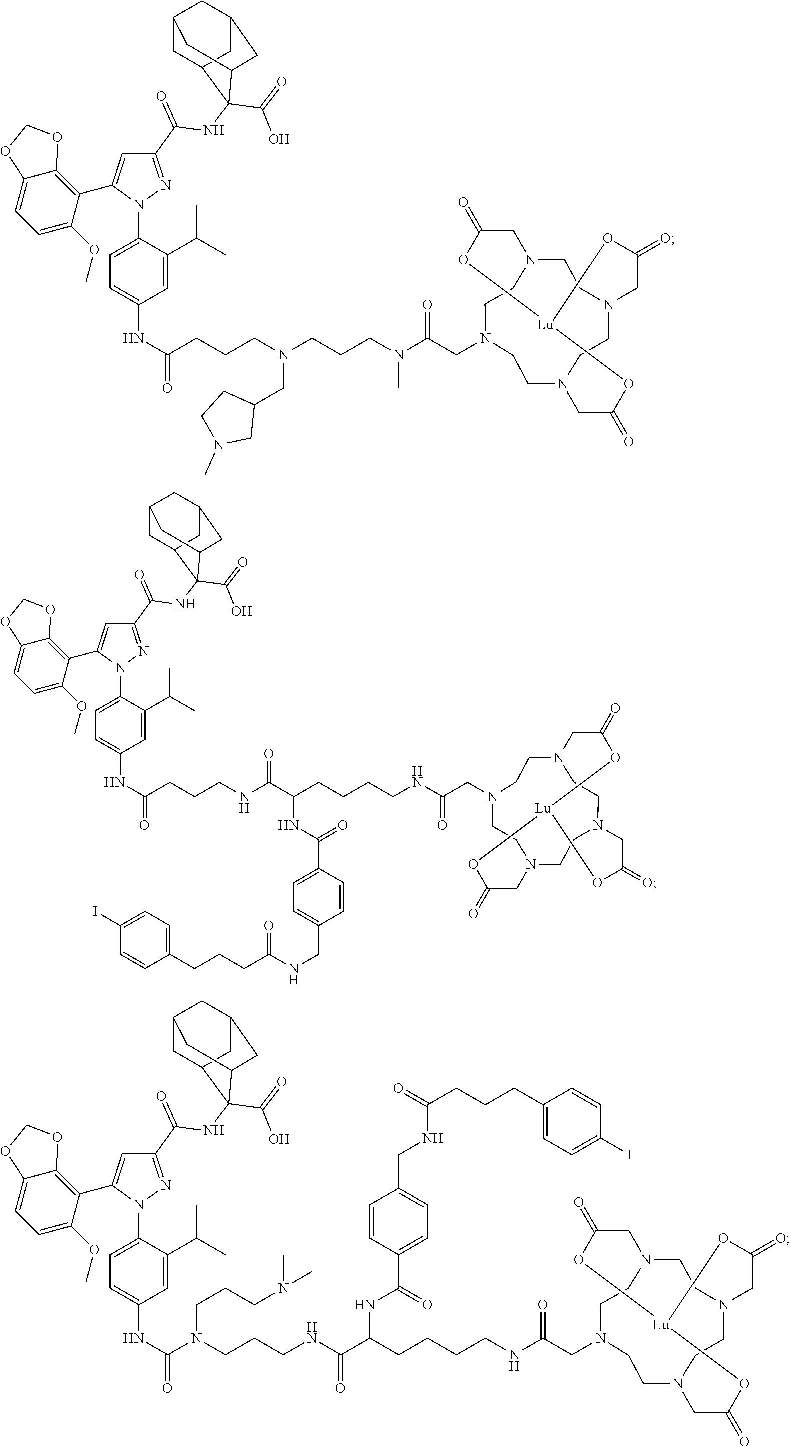

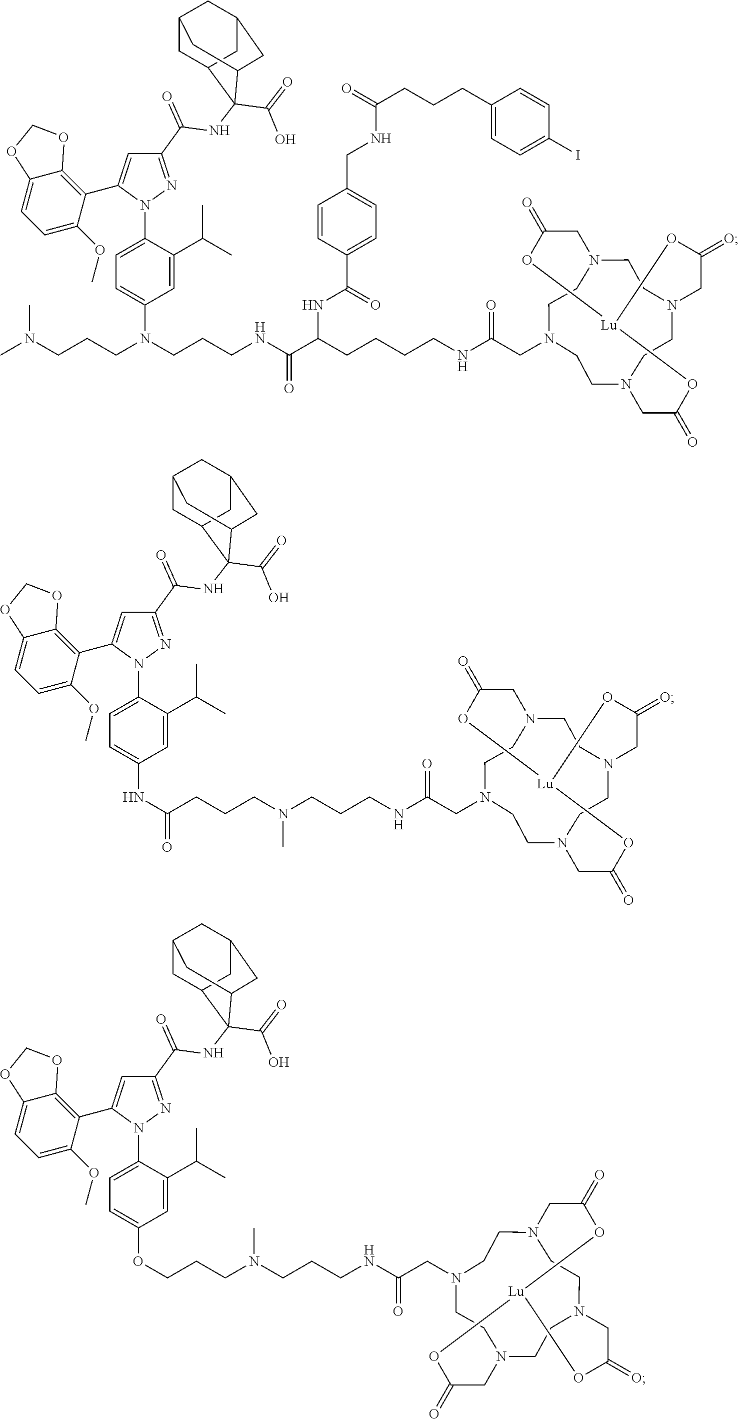

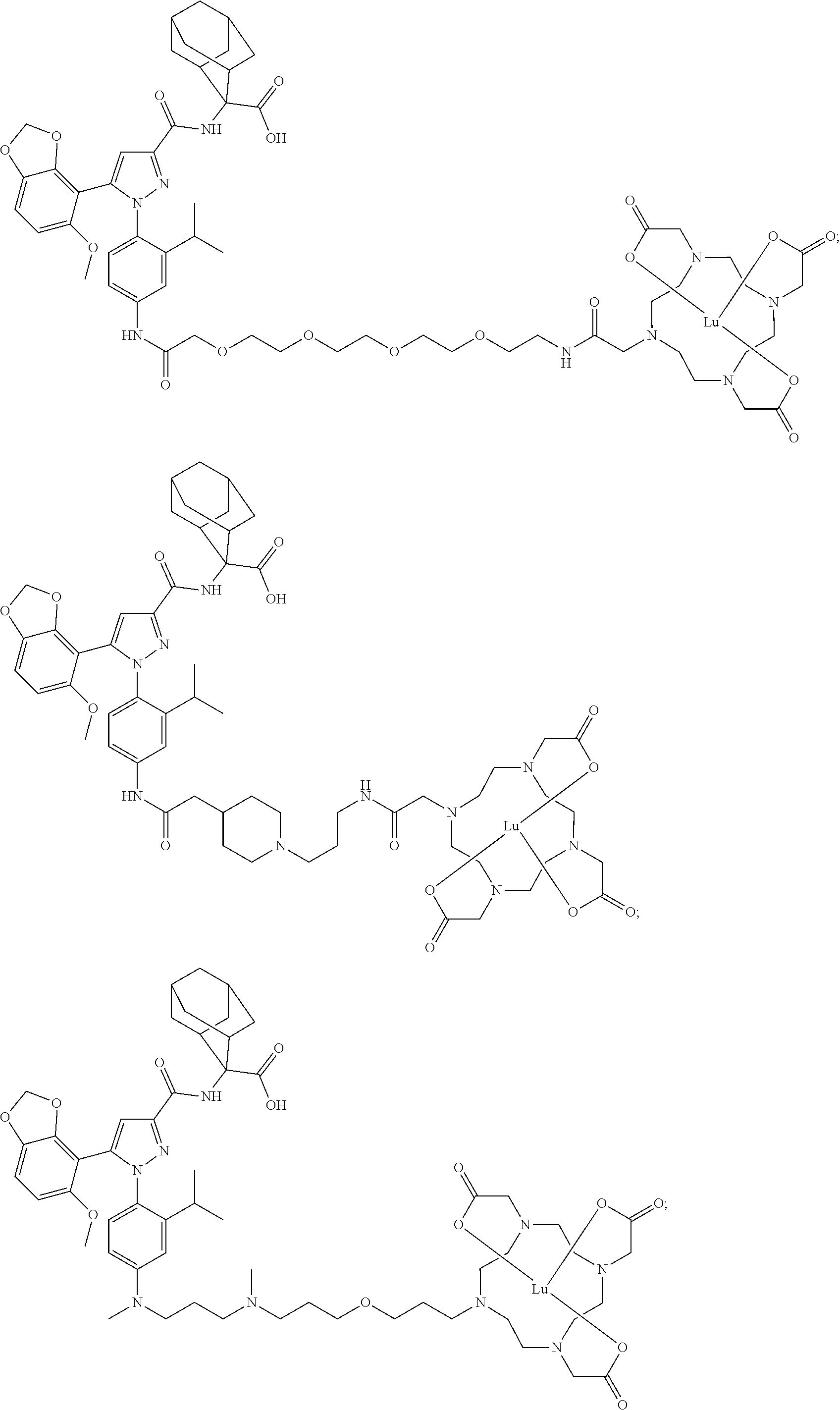

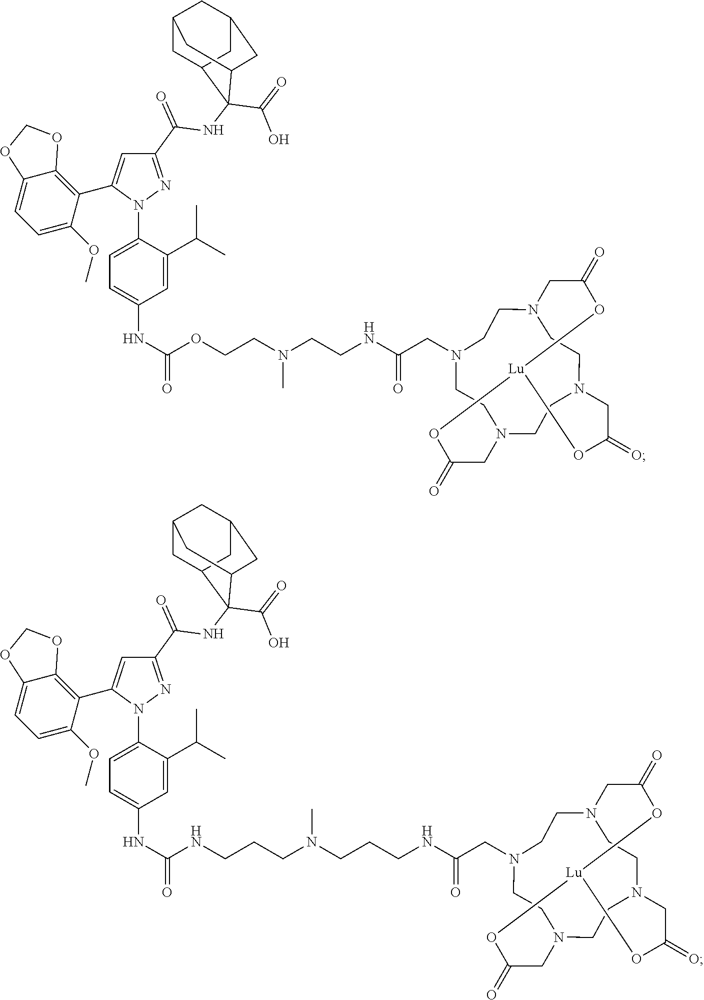

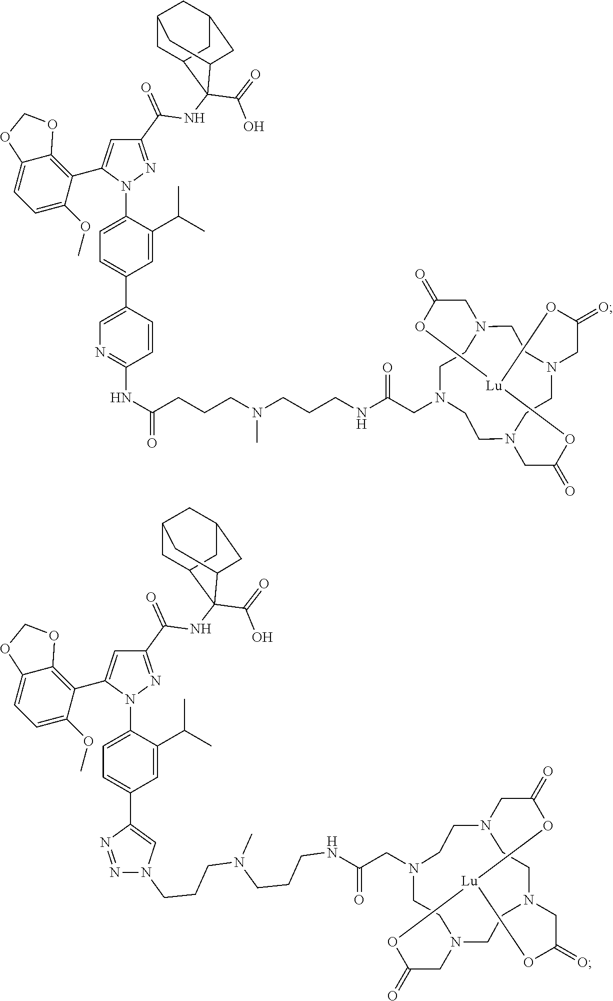

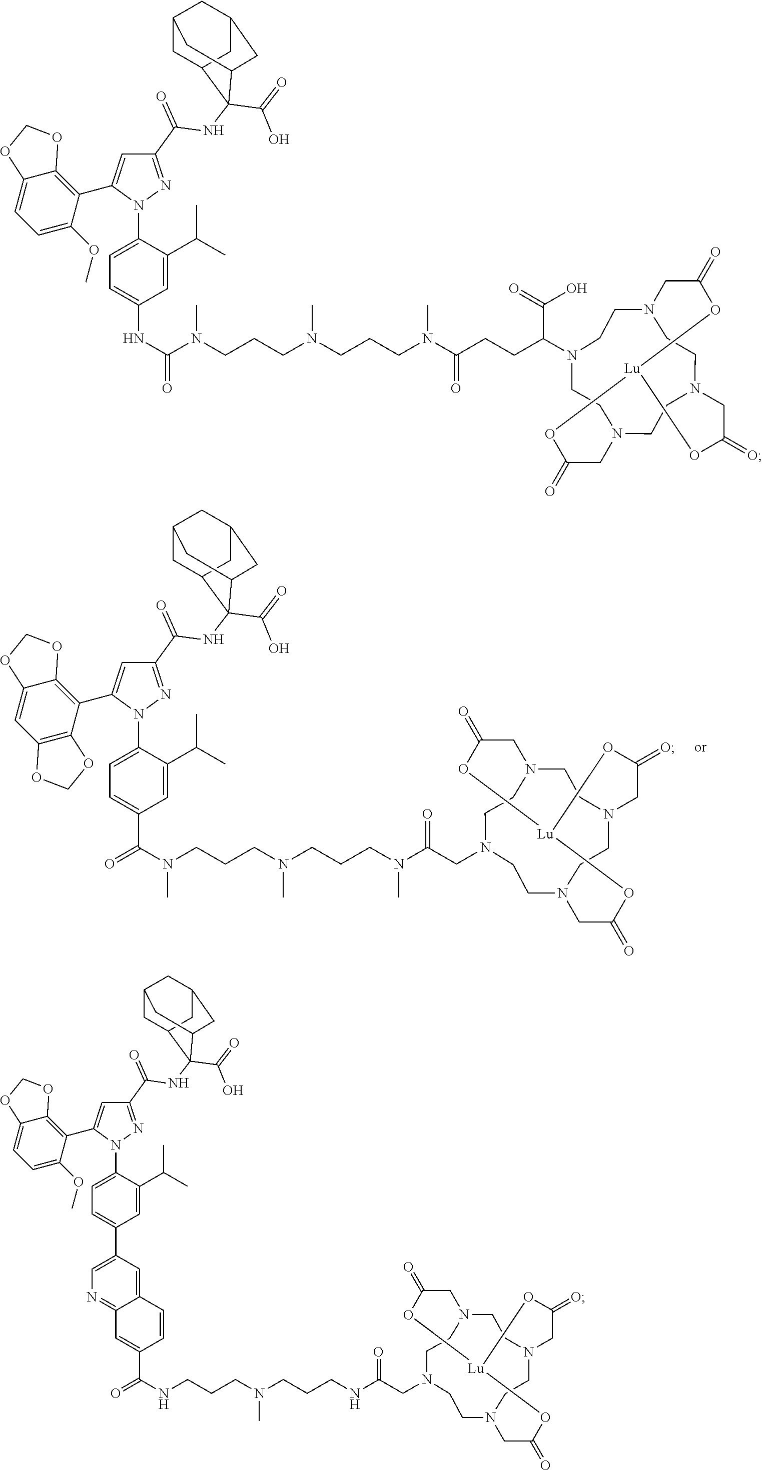

or a pharmaceutically acceptable salt thereof.

The present invention also provides a compound that is

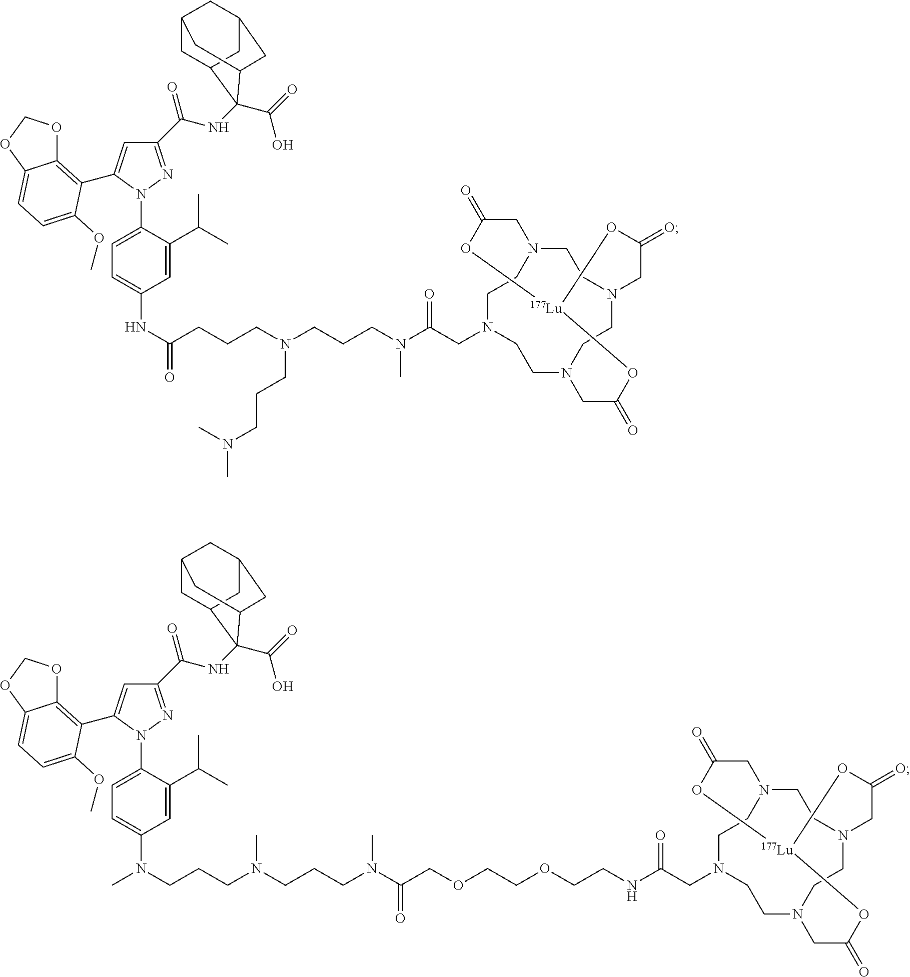

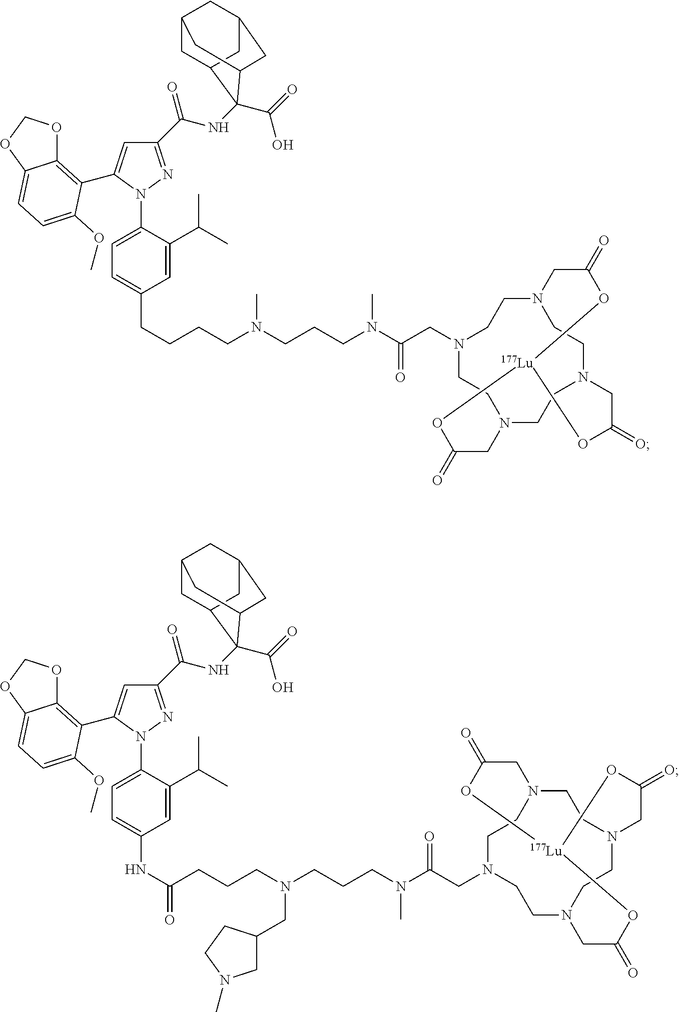

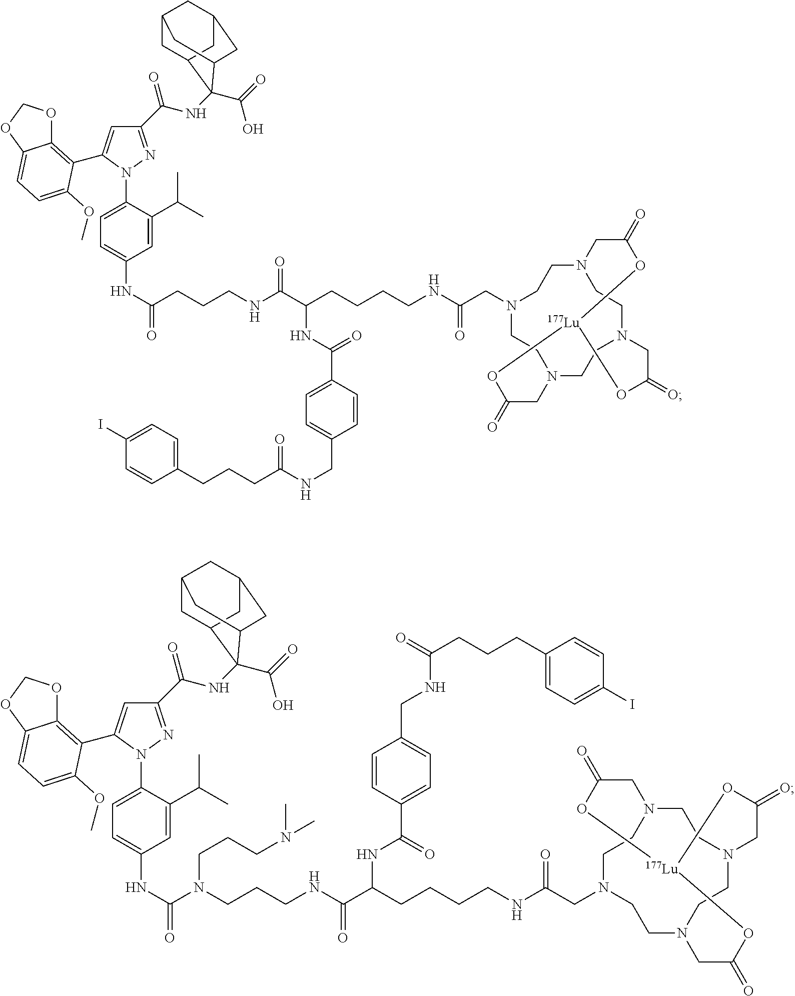

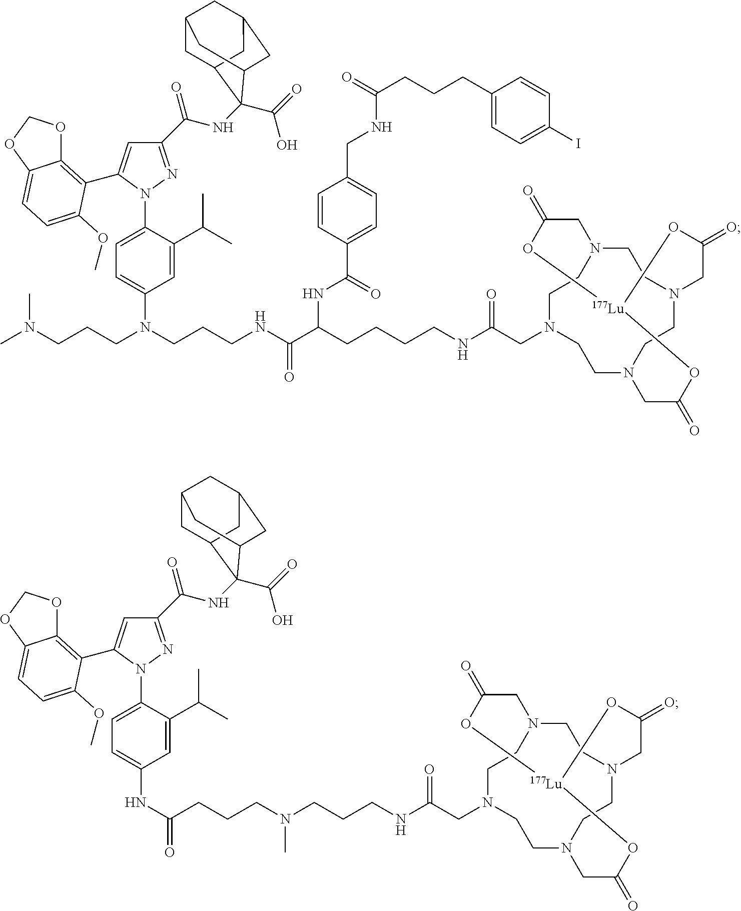

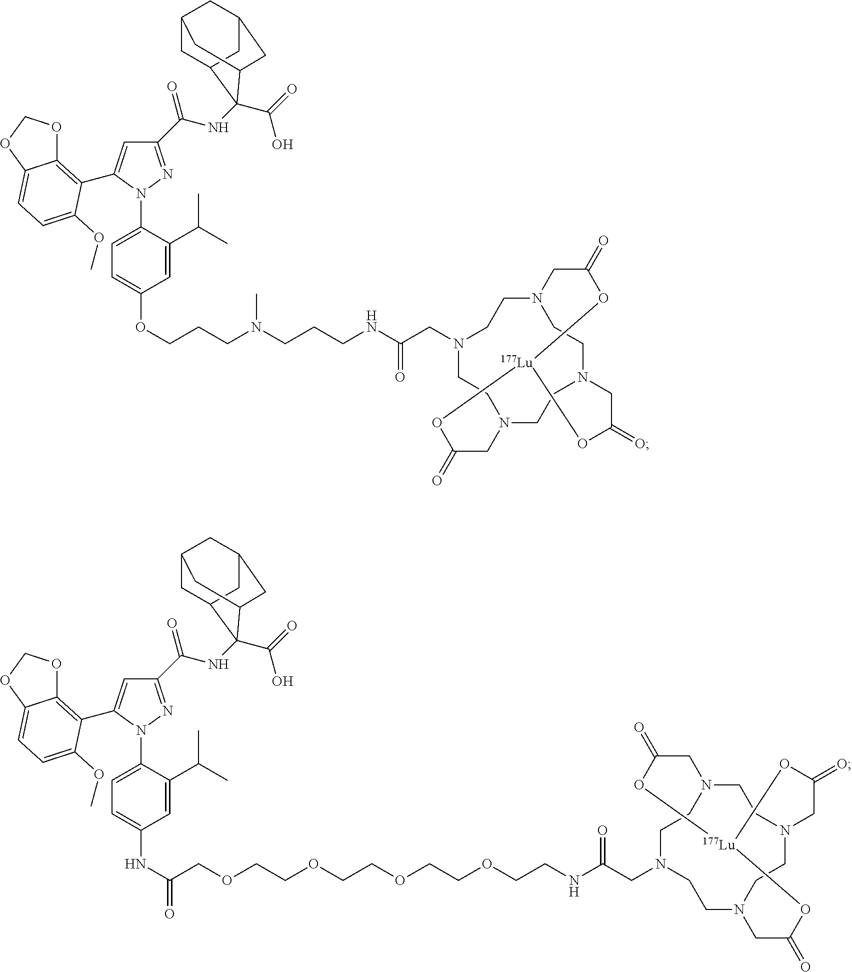

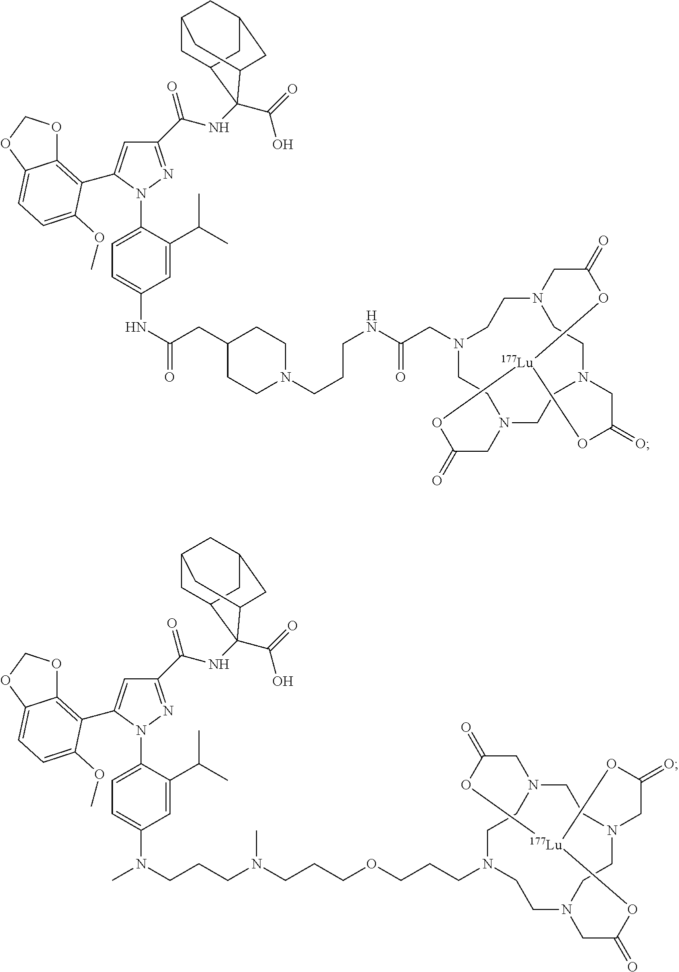

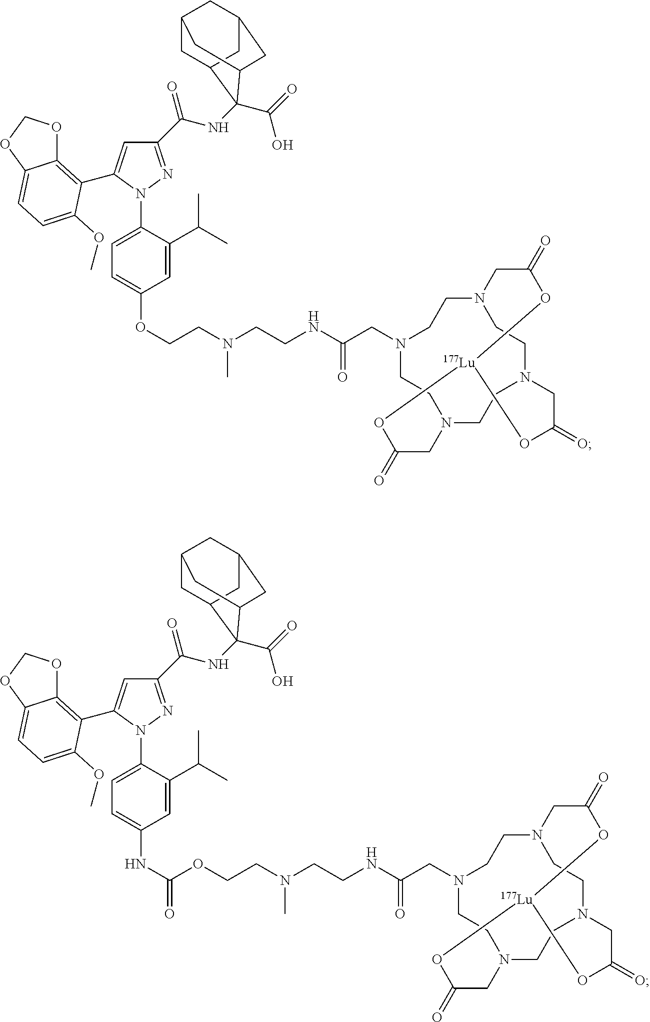

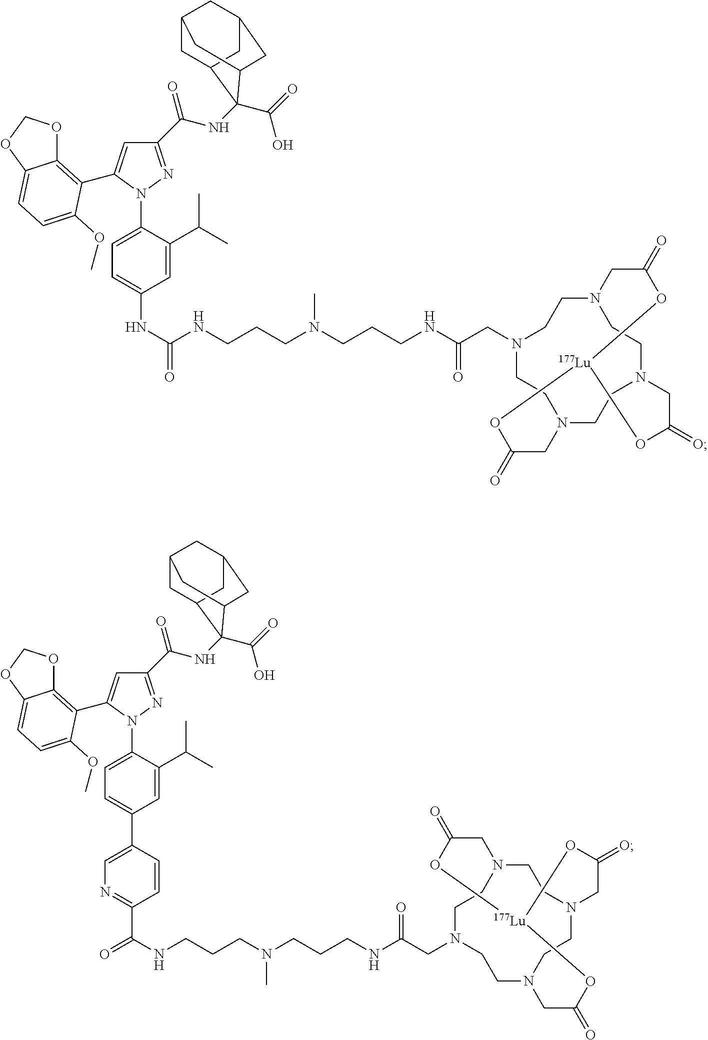

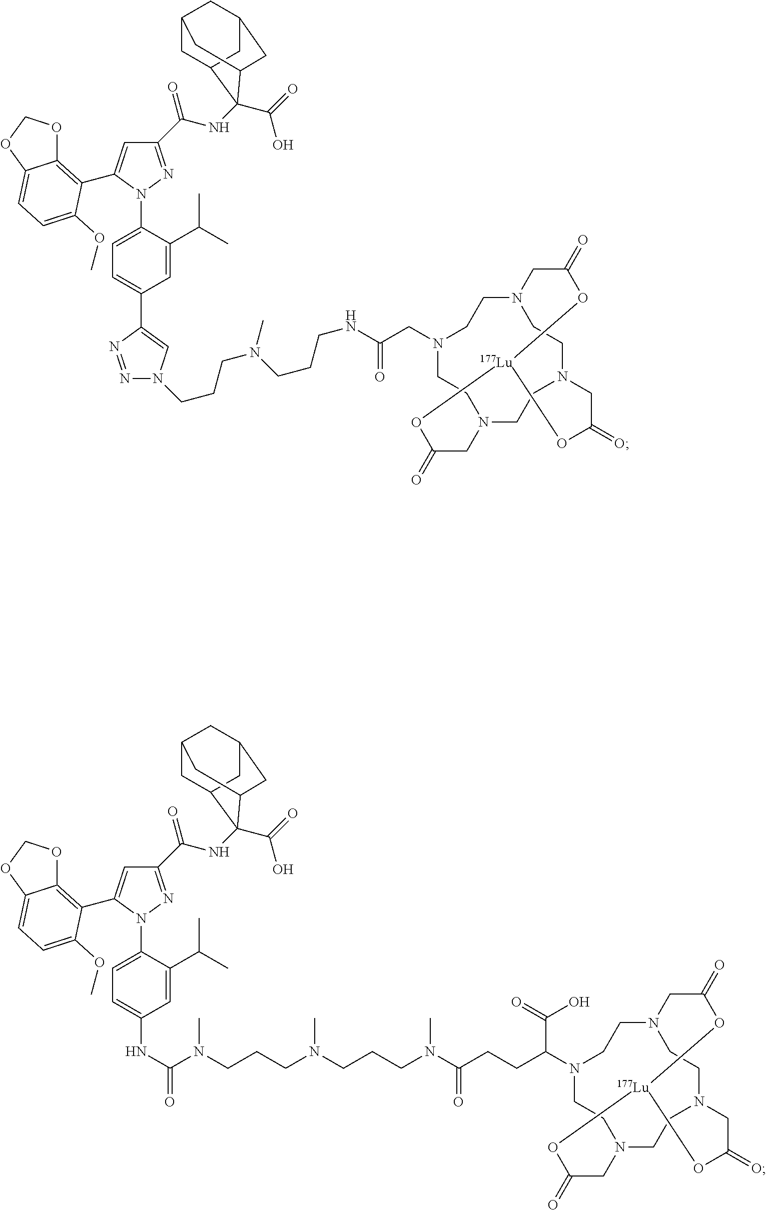

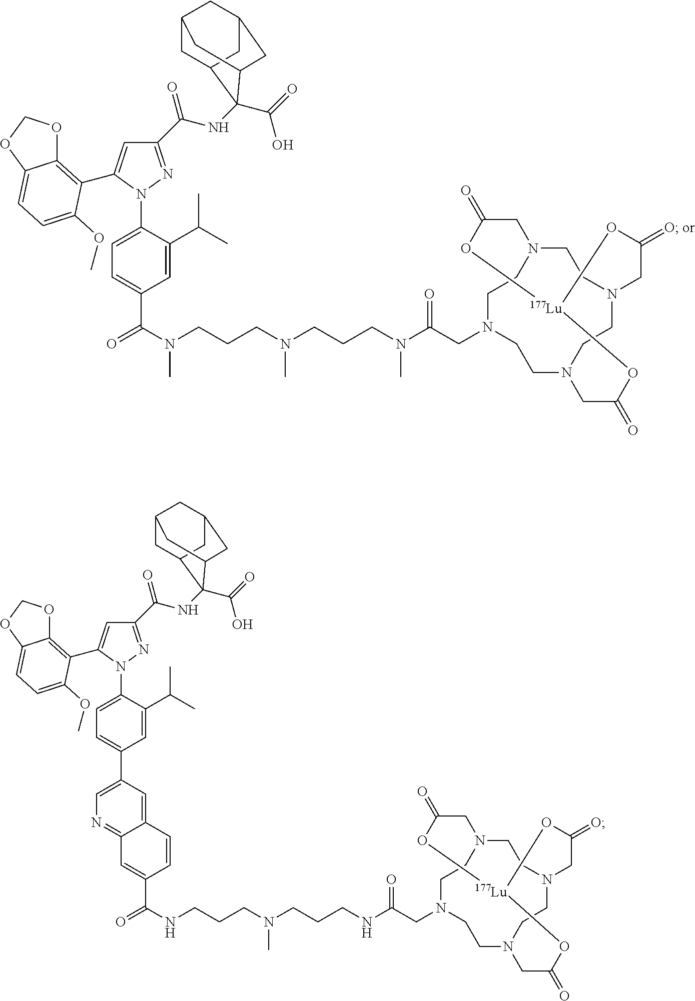

or a pharmaceutically acceptable salt thereof.

The present invention also provides a compound that is

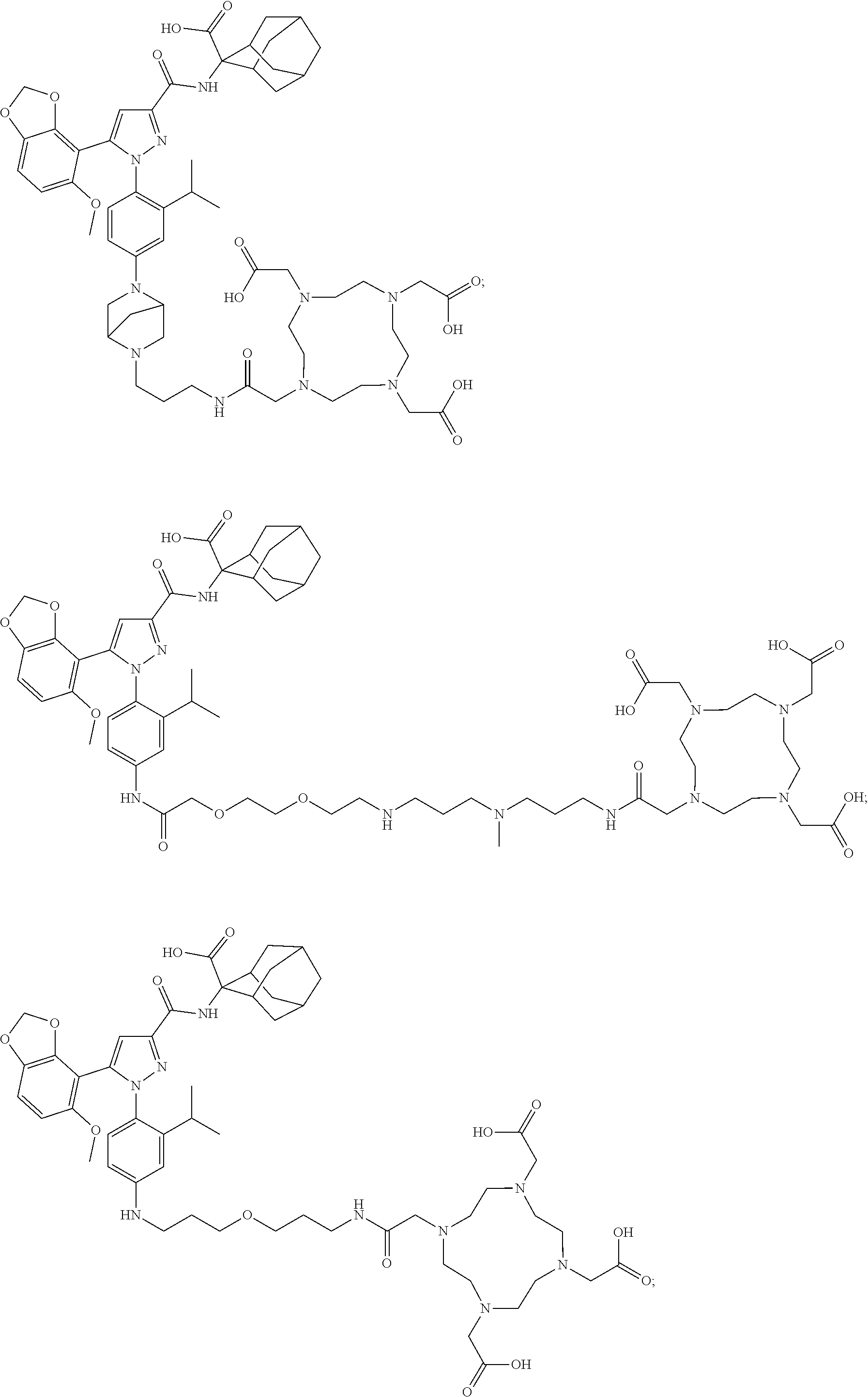 [SMILES: COc1ccc2c(c1-c1cc(C(=O)NC3(C(=O)O)C4CC5CC(C4)CC3C5)nn1-c1ccc(N3CC4CC3CN4CCCNC(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(NC(=O)COCCOCCNCCCN(C)CCCNC(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(NCCCOCCCNC(=O)CN3CCN(CC(=O)O)CCN(CC(=O)O)CCN(CC(=O)O)CC3)cc1C(C)C)OCO2]

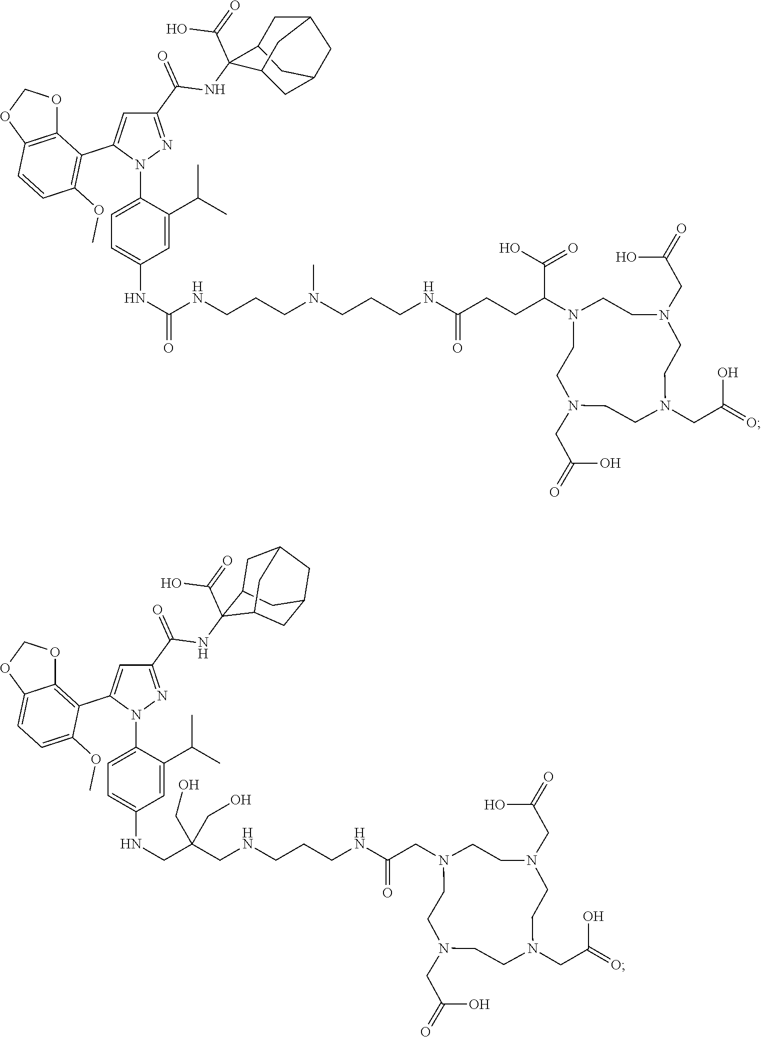 [SMILES: COc1ccc2c(c1-c1cc(C(=O)NC3(C(=O)O)C4CC5CC(C4)CC3C5)nn1-c1ccc(NC(=O)NCCCN(C)CCCNC(=O)CCC(C(=O)O)N3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(NCC(CO)(CO)CNCCCNC(=O)CN3CCN(CC(=O)O)CCN(CC(=O)O)CCN(CC(=O)O)CC3)cc1C(C)C)OCO2]

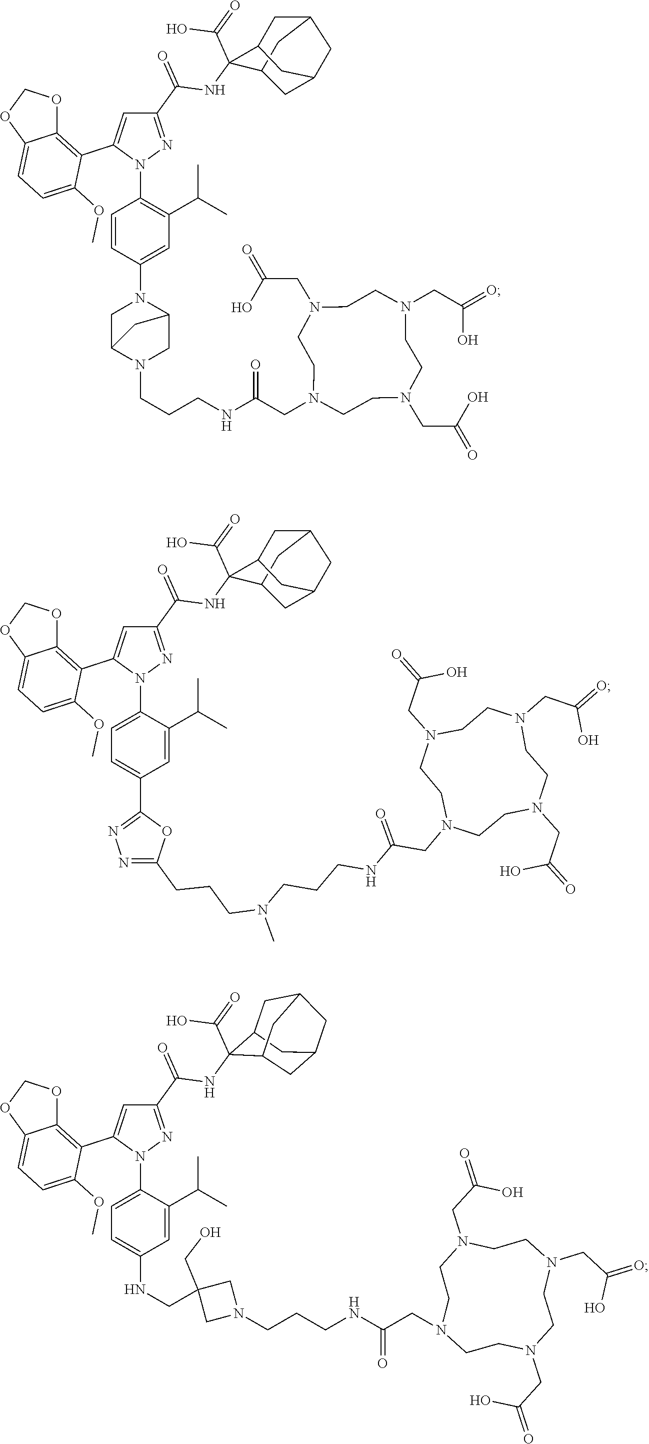 [SMILES: COc1ccc2c(c1-c1cc(C(=O)NC3(C(=O)O)C4CC5CC(C4)CC3C5)nn1-c1ccc(N3CC4CC3CN4CCCNC(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(-c3nnc(CCCN(C)CCCNC(=O)CN4CCN(CC(=O)O)CCN(CC(=O)O)CCN(CC(=O)O)CC4)o3)cc1C(C)C)OCO2 ; COc1ccc2c(c1-c1cc(C(=O)NC3(C(=O)O)C4CC5CC(C4)CC3C5)nn1-c1ccc(NCC3(CO)CN(CCCNC(=O)CN4CCN(CC(=O)O)CCN(CC(=O)O)CCN(CC(=O)O)CC4)C3)cc1C(C)C)OCO2]

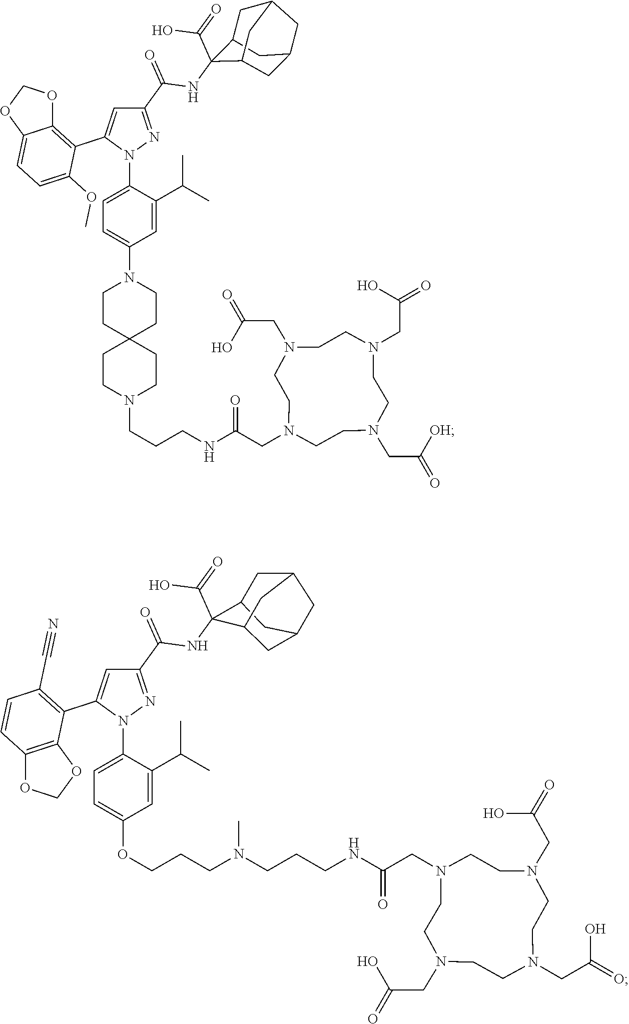 [SMILES: COc1ccc2c(c1-c1cc(C(=O)NC3(C(=O)O)C4CC5CC(C4)CC3C5)nn1-c1ccc(N3CCC4(CCN(CCCNC(=O)CN5CCN(CC(=O)O)CCN(CC(=O)O)CCN(CC(=O)O)CC5)CC4)CC3)cc1C(C)C)OCO2 ; CC(C)c1cc(OCCCN(C)CCCNC(=O)CN2CCN(CC(=O)O)CCN(CC(=O)O)CCN(CC(=O)O)CC2)ccc1-n1nc(C(=O)NC2(C(=O)O)C3CC4CC(C3)CC2C4)cc1-c1c(C#N)ccc2c1OCO2]

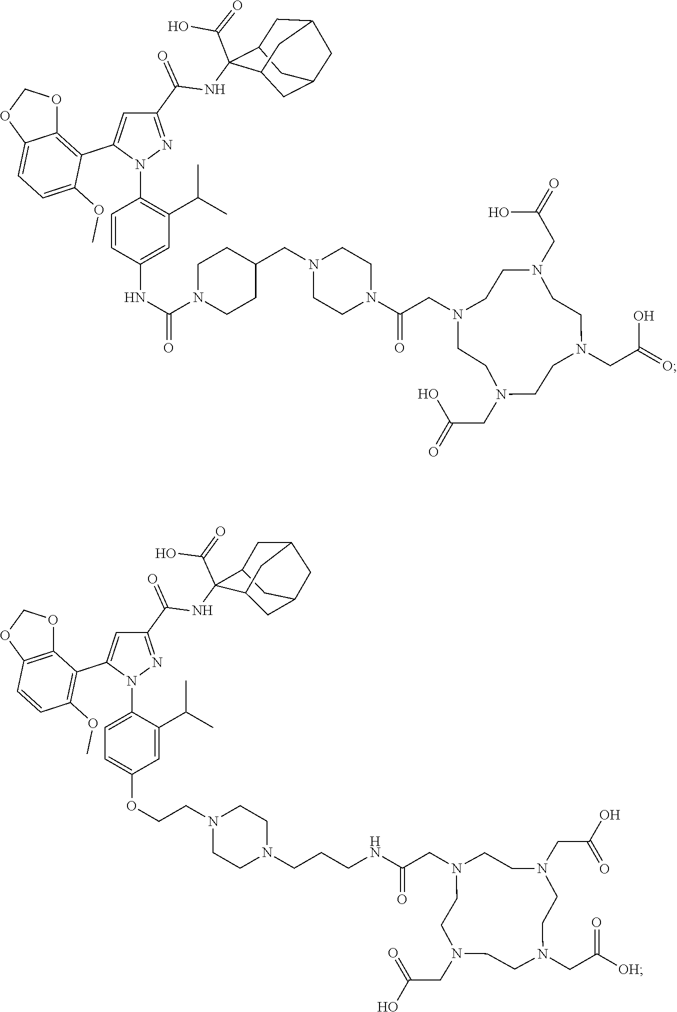 [SMILES: COc1ccc2c(c1-c1cc(C(=O)NC3(C(=O)O)C4CC5CC(C4)CC3C5)nn1-c1ccc(NC(=O)N3CCC(CN4CCN(C(=O)CN5CCN(CC(=O)O)CCN(CC(=O)O)CCN(CC(=O)O)CC5)CC4)CC3)cc1C(C)C)OCO2 ; COc1ccc2c(c1-c1cc(C(=O)NC3(C(=O)O)C4CC5CC(C4)CC3C5)nn1-c1ccc(OCCN3CCN(CCCNC(=O)CN4CCN(CC(=O)O)CCN(CC(=O)O)CCN(CC(=O)O)CC4)CC3)cc1C(C)C)OCO2]

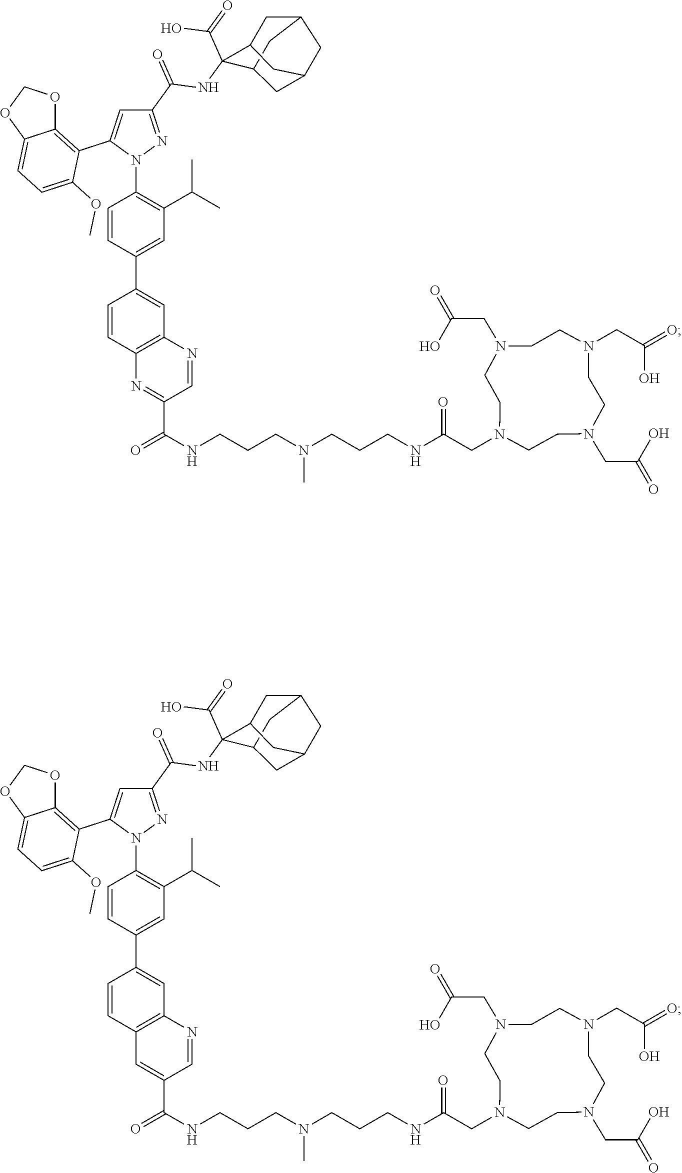 [SMILES: COc1ccc2c(c1-c1cc(C(=O)NC3(C(=O)O)C4CC5CC(C4)CC3C5)nn1-c1ccc(-c3ccc4nc(C(=O)NCCCN(C)CCCNC(=O)CN5CCN(CC(=O)O)CCN(CC(=O)O)CCN(CC(=O)O)CC5)cnc4c3)cc1C(C)C)OCO2 ; COc1ccc2c(c1-c1cc(C(=O)NC3(C(=O)O)C4CC5CC(C4)CC3C5)nn1-c1ccc(-c3ccc4cc(C(=O)NCCCN(C)CCCNC(=O)CN5CCN(CC(=O)O)CCN(CC(=O)O)CCN(CC(=O)O)CC5)cnc4c3)cc1C(C)C)OCO2]

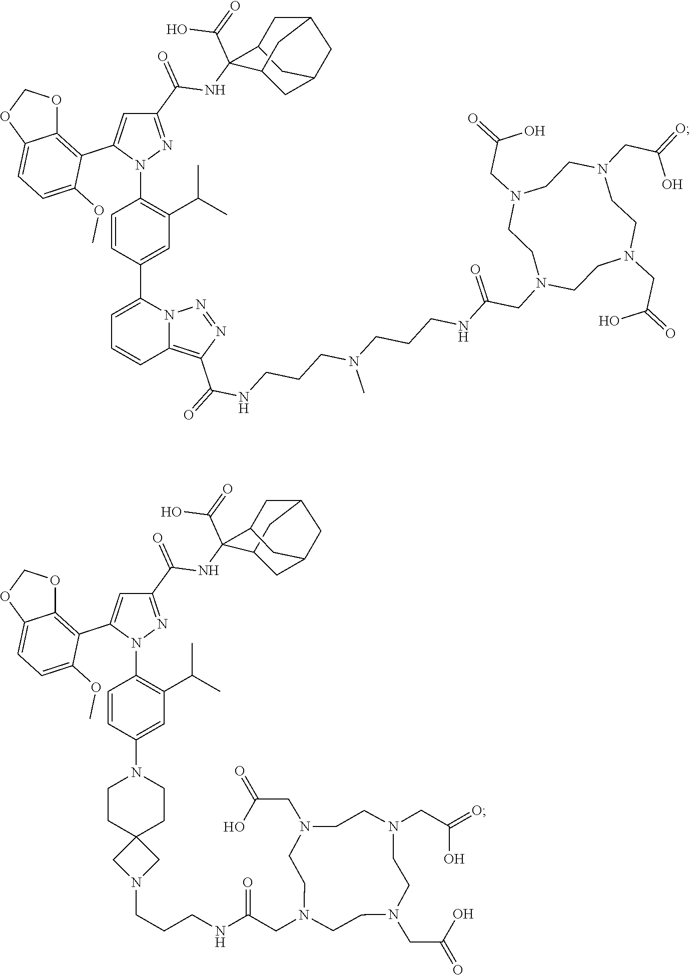 [SMILES: COc1ccc2c(c1-c1cc(C(=O)NC3(C(=O)O)C4CC5CC(C4)CC3C5)nn1-c1ccc(-c3cccc4c(C(=O)NCCCN(C)CCCNC(=O)CN5CCN(CC(=O)O)CCN(CC(=O)O)CCN(CC(=O)O)CC5)nnn34)cc1C(C)C)OCO2 ; COc1ccc2c(c1-c1cc(C(=O)NC3(C(=O)O)C4CC5CC(C4)CC3C5)nn1-c1ccc(N3CCC4(CC3)CN(CCCNC(=O)CN3CCN(CC(=O)O)CCN(CC(=O)O)CCN(CC(=O)O)CC3)C4)cc1C(C)C)OCO2]

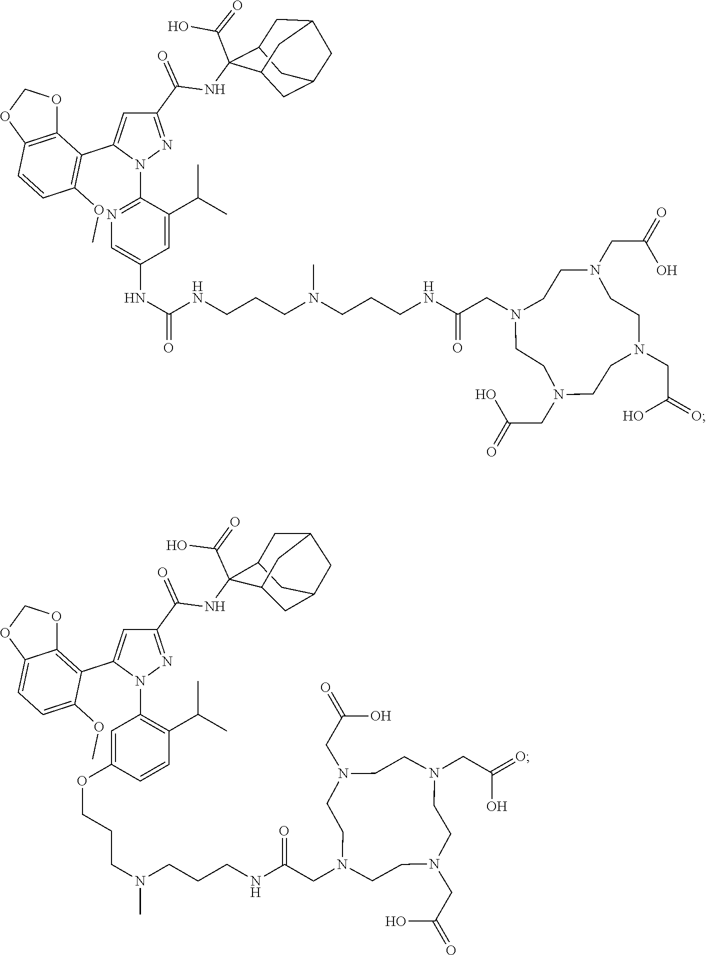 [SMILES: COc1ccc2c(c1-c1cc(C(=O)NC3(C(=O)O)C4CC5CC(C4)CC3C5)nn1-c1ncc(NC(=O)NCCCN(C)CCCNC(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1cc(OCCCN(C)CCCNC(=O)CN3CCN(CC(=O)O)CCN(CC(=O)O)CCN(CC(=O)O)CC3)ccc1C(C)C)OCO2]

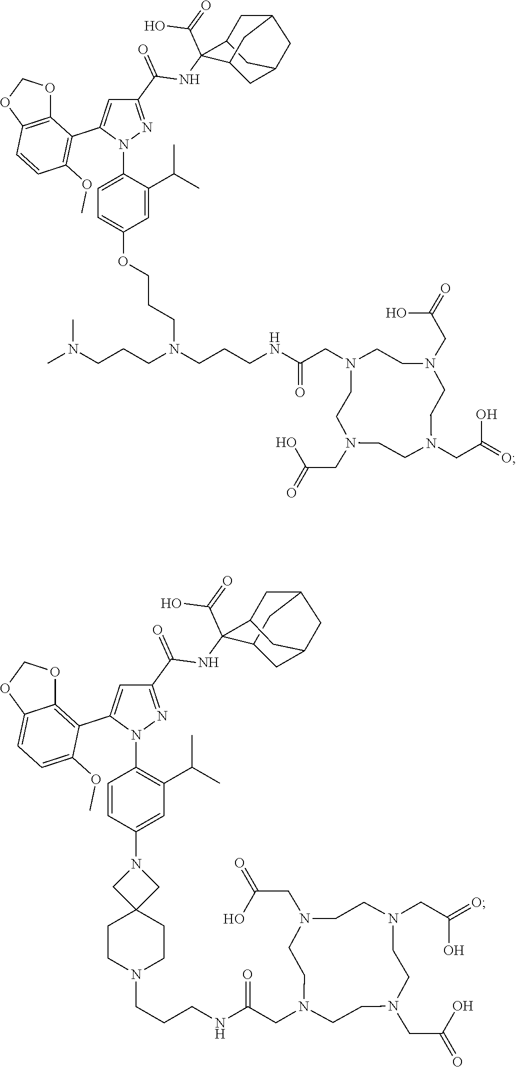 [SMILES: COc1ccc2c(c1-c1cc(C(=O)NC3(C(=O)O)C4CC5CC(C4)CC3C5)nn1-c1ccc(OCCCN(CCCNC(=O)CN3CCN(CC(=O)O)CCN(CC(=O)O)CCN(CC(=O)O)CC3)CCCN(C)C)cc1C(C)C)OCO2 ; COc1ccc2c(c1-c1cc(C(=O)NC3(C(=O)O)C4CC5CC(C4)CC3C5)nn1-c1ccc(N3CC4(CCN(CCCNC(=O)CN5CCN(CC(=O)O)CCN(CC(=O)O)CCN(CC(=O)O)CC5)CC4)C3)cc1C(C)C)OCO2]

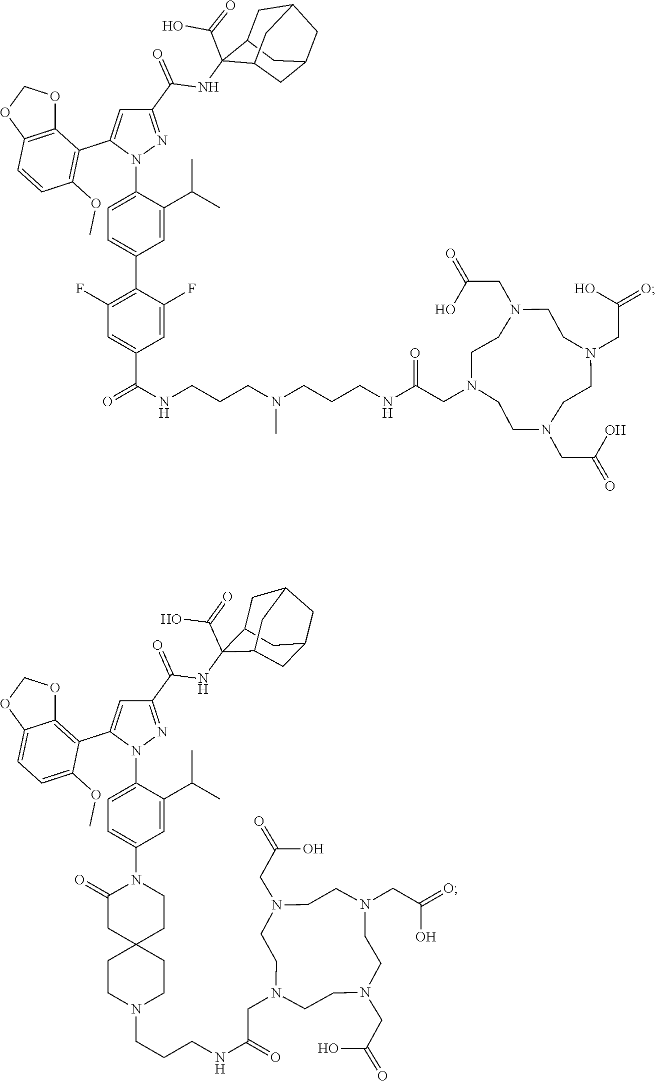 [SMILES: COc1ccc2c(c1-c1cc(C(=O)NC3(C(=O)O)C4CC5CC(C4)CC3C5)nn1-c1ccc(-c3c(F)cc(C(=O)NCCCN(C)CCCNC(=O)CN4CCN(CC(=O)O)CCN(CC(=O)O)CCN(CC(=O)O)CC4)cc3F)cc1C(C)C)OCO2 ; COc1ccc2c(c1-c1cc(C(=O)NC3(C(=O)O)C4CC5CC(C4)CC3C5)nn1-c1ccc(N3CCC4(CCN(CCCNC(=O)CN5CCN(CC(=O)O)CCN(CC(=O)O)CCN(CC(=O)O)CC5)CC4)CC3=O)cc1C(C)C)OCO2]

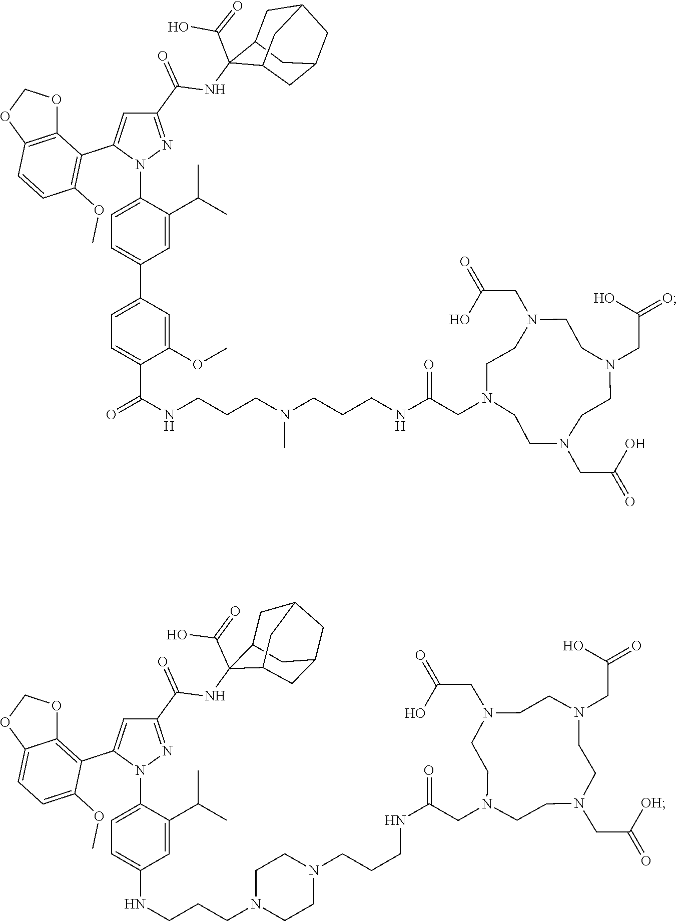 [SMILES: COc1cc(-c2ccc(-n3nc(C(=O)NC4(C(=O)O)C5CC6CC(C5)CC4C6)cc3-c3c(OC)ccc4c3OCO4)c(C(C)C)c2)ccc1C(=O)NCCCN(C)CCCNC(=O)CN1CCN(CC(=O)O)CCN(CC(=O)O)CCN(CC(=O)O)CC1 ; COc1ccc2c(c1-c1cc(C(=O)NC3(C(=O)O)C4CC5CC(C4)CC3C5)nn1-c1ccc(NCCCN3CCN(CCCNC(=O)CN4CCN(CC(=O)O)CCN(CC(=O)O)CCN(CC(=O)O)CC4)CC3)cc1C(C)C)OCO2]

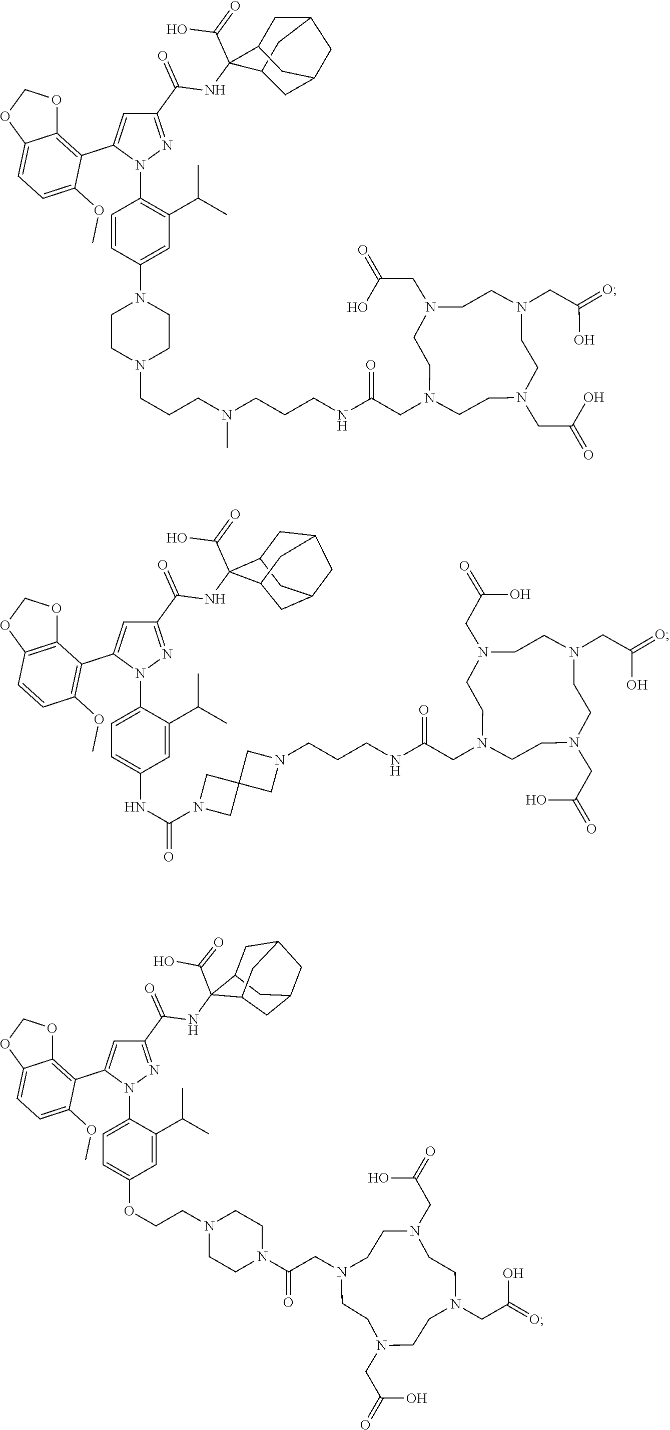 [SMILES: COc1ccc2c(c1-c1cc(C(=O)NC3(C(=O)O)C4CC5CC(C4)CC3C5)nn1-c1ccc(N3CCN(CCCN(C)CCCNC(=O)CN4CCN(CC(=O)O)CCN(CC(=O)O)CCN(CC(=O)O)CC4)CC3)cc1C(C)C)OCO2 ; COc1ccc2c(c1-c1cc(C(=O)NC3(C(=O)O)C4CC5CC(C4)CC3C5)nn1-c1ccc(NC(=O)N3CC4(CN(CCCNC(=O)CN5CCN(CC(=O)O)CCN(CC(=O)O)CCN(CC(=O)O)CC5)C4)C3)cc1C(C)C)OCO2 ; COc1ccc2c(c1-c1cc(C(=O)NC3(C(=O)O)C4CC5CC(C4)CC3C5)nn1-c1ccc(OCCN3CCN(C(=O)CN4CCN(CC(=O)O)CCN(CC(=O)O)CCN(CC(=O)O)CC4)CC3)cc1C(C)C)OCO2]

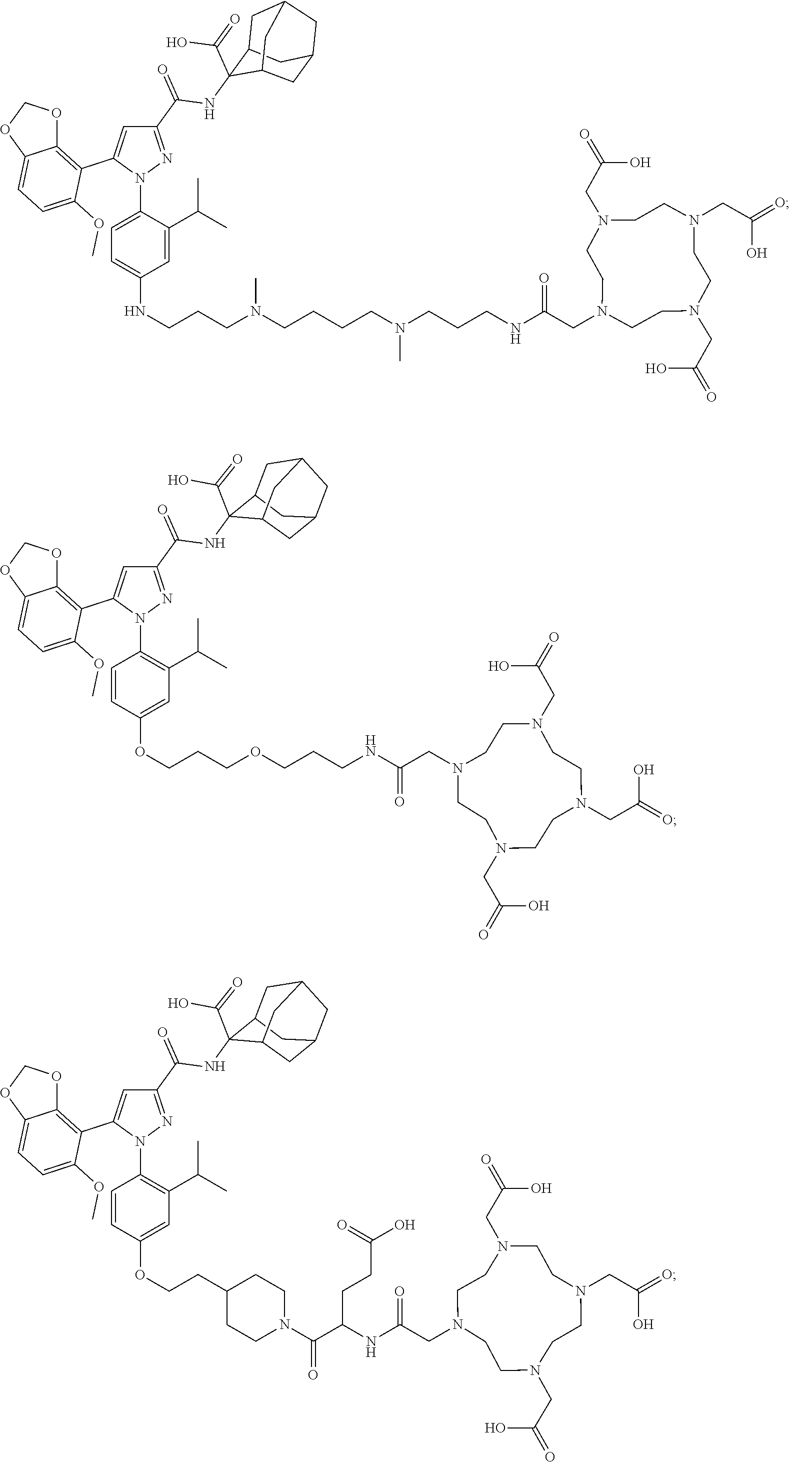 [SMILES: COc1ccc2c(c1-c1cc(C(=O)NC3(C(=O)O)C4CC5CC(C4)CC3C5)nn1-c1ccc(NCCCN(C)CCCCN(C)CCCNC(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(OCCCOCCCNC(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(OCCC3CCN(C(=O)C(CCC(=O)O)NC(=O)CN4CCN(CC(=O)O)CCN(CC(=O)O)CCN(CC(=O)O)CC4)CC3)cc1C(C)C)OCO2]

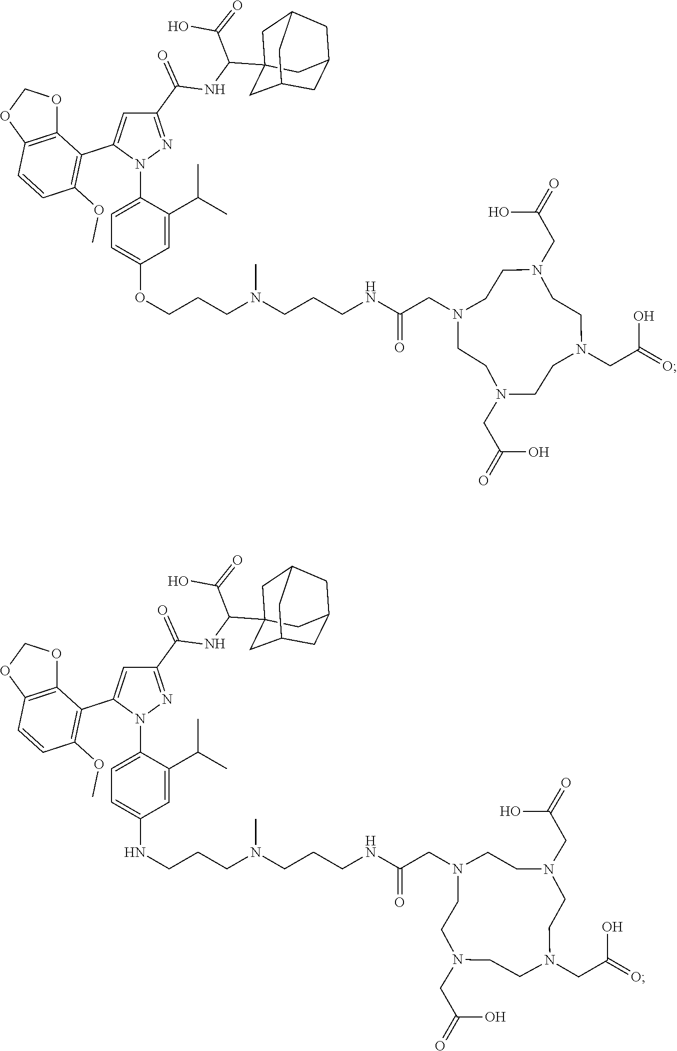 [SMILES: COc1ccc2c(c1-c1cc(C(=O)NC(C(=O)O)C34CC5CC(CC(C5)C3)C4)nn1-c1ccc(OCCCN(C)CCCNC(=O)CN3CCN(CC(=O)O)CCN(CC(=O)O)CCN(CC(=O)O)CC3)cc1C(C)C)OCO2 ; COc1ccc2c(c1-c1cc(C(=O)NC(C(=O)O)C34CC5CC(CC(C5)C3)C4)nn1-c1ccc(NCCCN(C)CCCNC(=O)CN3CCN(CC(=O)O)CCN(CC(=O)O)CCN(CC(=O)O)CC3)cc1C(C)C)OCO2]

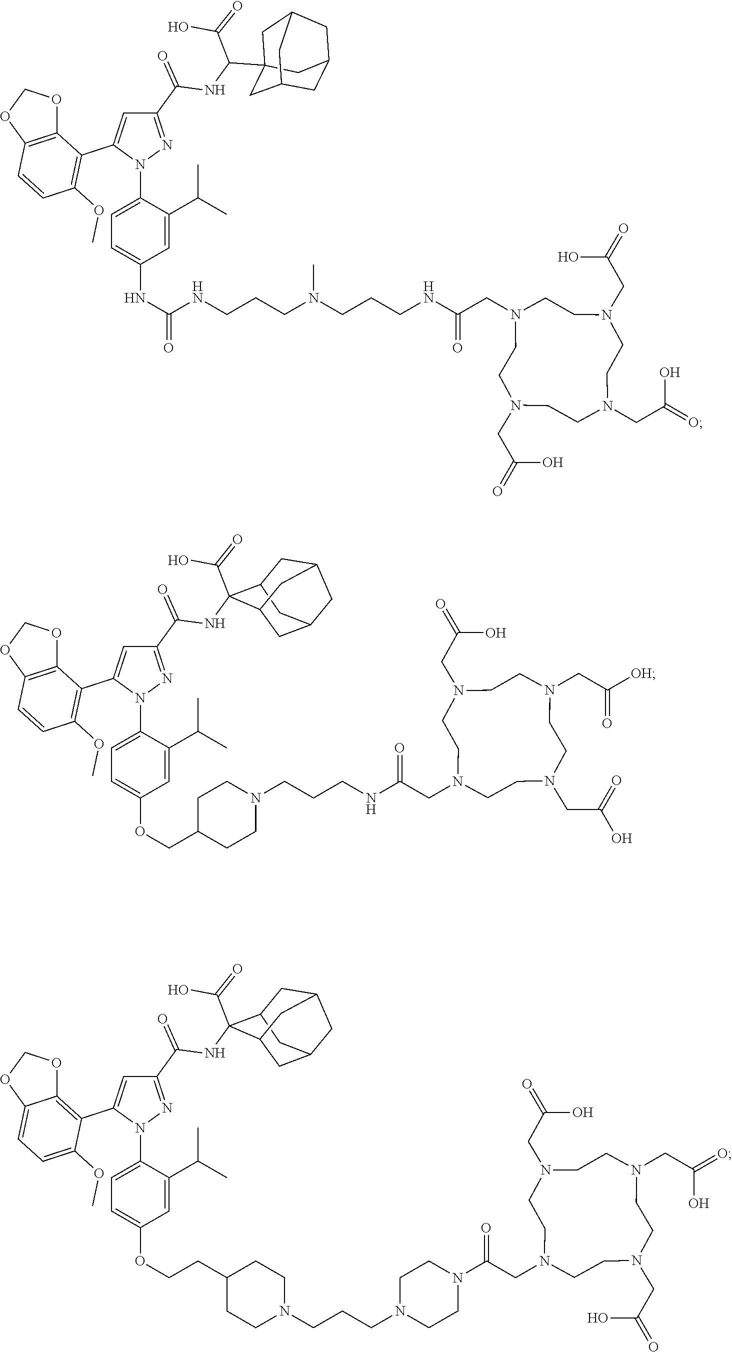 [SMILES: COc1ccc2c(c1-c1cc(C(=O)NC(C(=O)O)C34CC5CC(CC(C5)C3)C4)nn1-c1ccc(NC(=O)NCCCN(C)CCCNC(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(OCC3CCN(CCCNC(=O)CN4CCN(CC(=O)O)CCN(CC(=O)O)CCN(CC(=O)O)CC4)CC3)cc1C(C)C)OCO2 ; COc1ccc2c(c1-c1cc(C(=O)NC3(C(=O)O)C4CC5CC(C4)CC3C5)nn1-c1ccc(OCCC3CCN(CCCN4CCN(C(=O)CN5CCN(CC(=O)O)CCN(CC(=O)O)CCN(CC(=O)O)CC5)CC4)CC3)cc1C(C)C)OCO2]

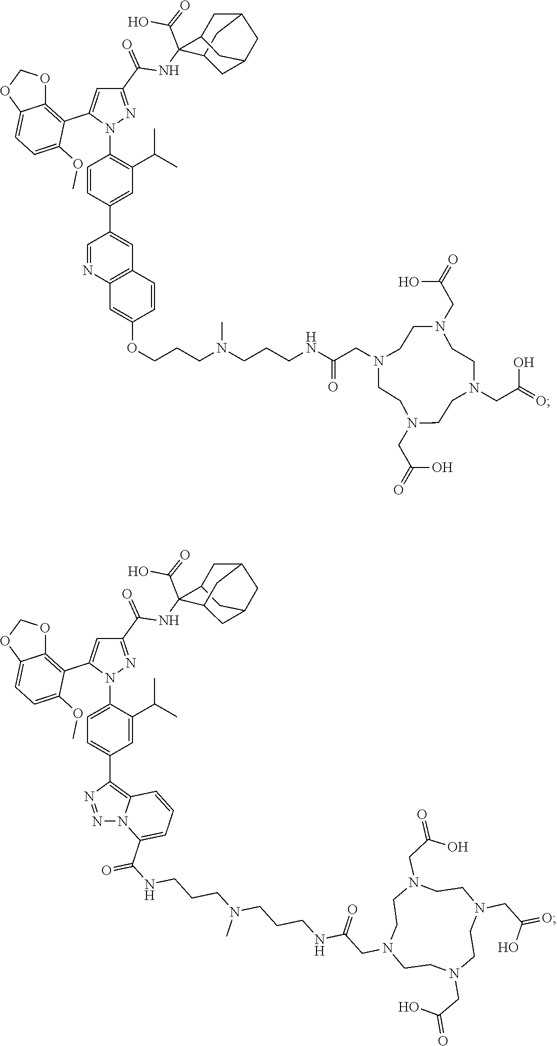 [SMILES: COc1ccc2c(c1-c1cc(C(=O)NC3(C(=O)O)C4CC5CC(C4)CC3C5)nn1-c1ccc(-c3cnc4cc(OCCCN(C)CCCNC(=O)CN5CCN(CC(=O)O)CCN(CC(=O)O)CCN(CC(=O)O)CC5)ccc4c3)cc1C(C)C)OCO2 ; COc1ccc2c(c1-c1cc(C(=O)NC3(C(=O)O)C4CC5CC(C4)CC3C5)nn1-c1ccc(-c3nnn4c(C(=O)NCCCN(C)CCCNC(=O)CN5CCN(CC(=O)O)CCN(CC(=O)O)CCN(CC(=O)O)CC5)cccc34)cc1C(C)C)OCO2]

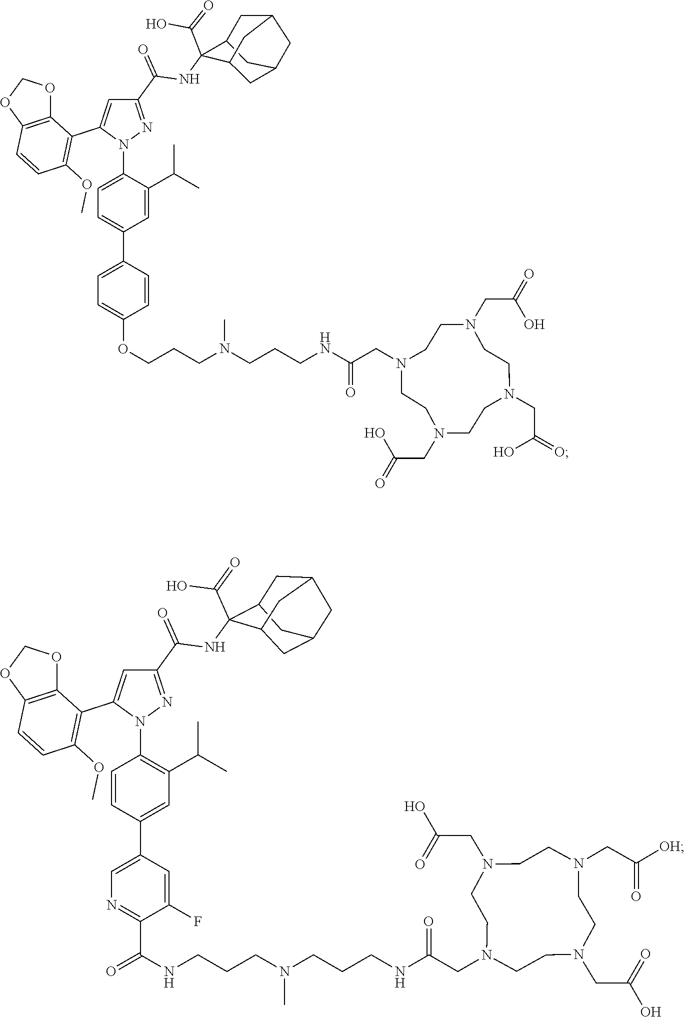 [SMILES: COc1ccc2c(c1-c1cc(C(=O)NC3(C(=O)O)C4CC5CC(C4)CC3C5)nn1-c1ccc(-c3ccc(OCCCN(C)CCCNC(=O)CN4CCN(CC(=O)O)CCN(CC(=O)O)CCN(CC(=O)O)CC4)cc3)cc1C(C)C)OCO2 ; COc1ccc2c(c1-c1cc(C(=O)NC3(C(=O)O)C4CC5CC(C4)CC3C5)nn1-c1ccc(-c3cnc(C(=O)NCCCN(C)CCCNC(=O)CN4CCN(CC(=O)O)CCN(CC(=O)O)CCN(CC(=O)O)CC4)c(F)c3)cc1C(C)C)OCO2]

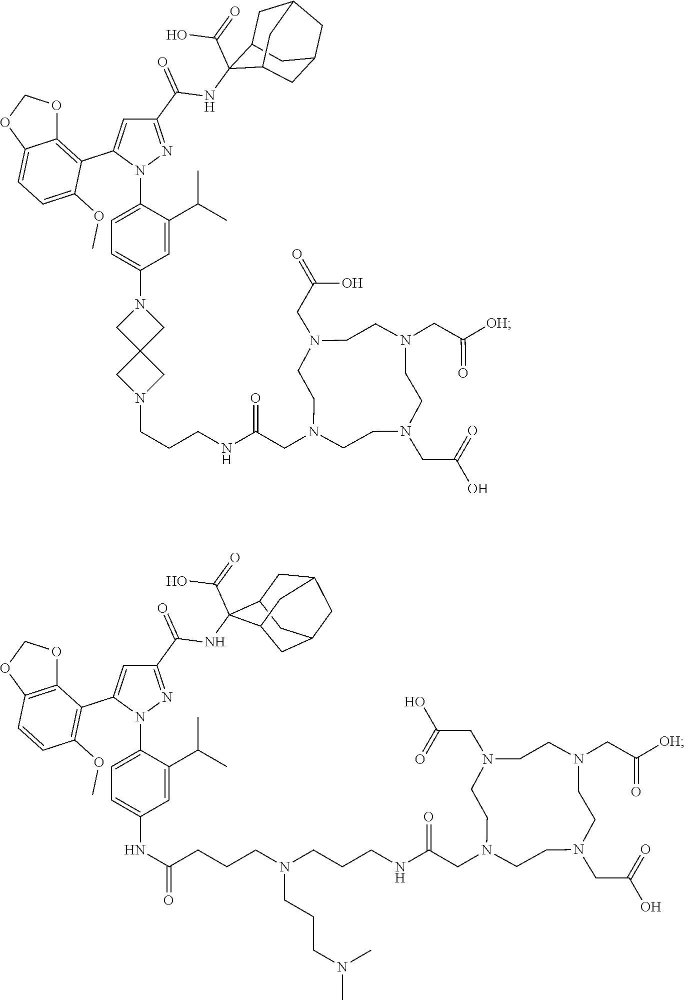 [SMILES: COc1ccc2c(c1-c1cc(C(=O)NC3(C(=O)O)C4CC5CC(C4)CC3C5)nn1-c1ccc(N3CC4(CN(CCCNC(=O)CN5CCN(CC(=O)O)CCN(CC(=O)O)CCN(CC(=O)O)CC5)C4)C3)cc1C(C)C)OCO2 ; COc1ccc2c(c1-c1cc(C(=O)NC3(C(=O)O)C4CC5CC(C4)CC3C5)nn1-c1ccc(NC(=O)CCCN(CCCNC(=O)CN3CCN(CC(=O)O)CCN(CC(=O)O)CCN(CC(=O)O)CC3)CCCN(C)C)cc1C(C)C)OCO2]

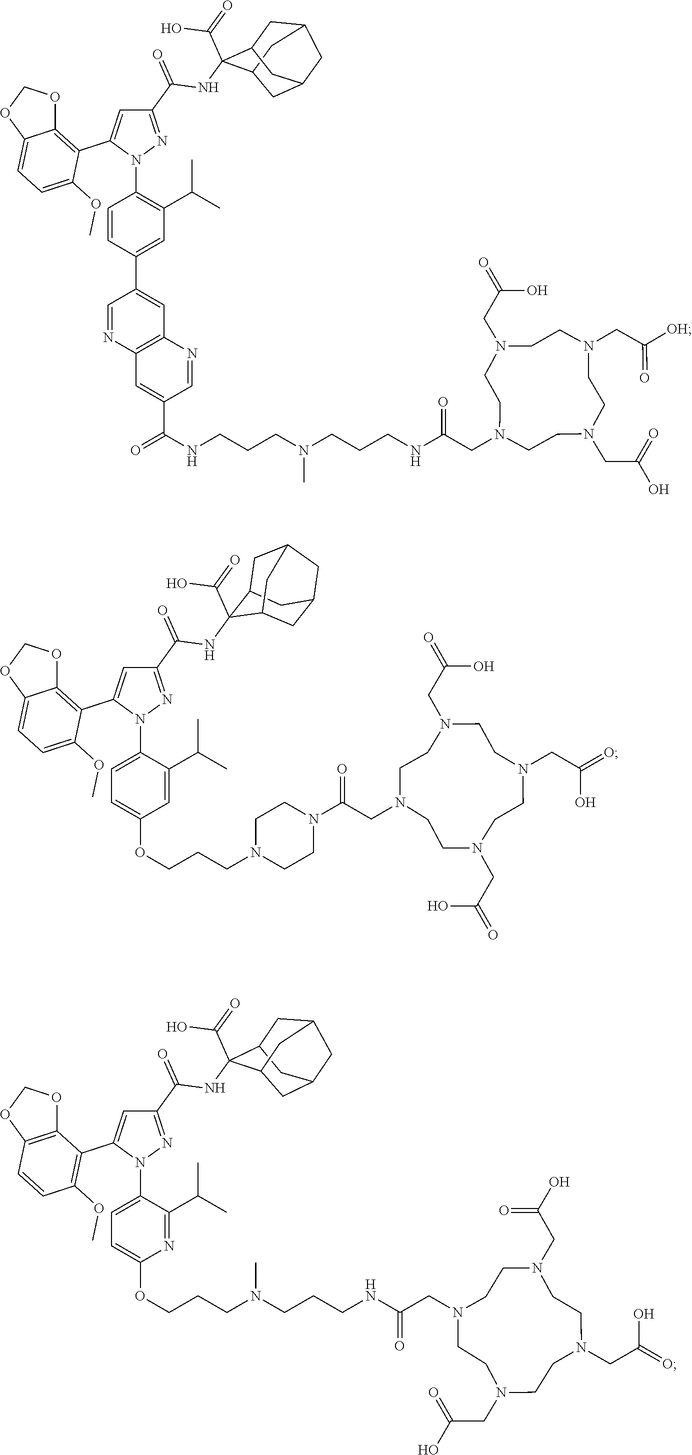 [SMILES: COc1ccc2c(c1-c1cc(C(=O)NC3(C(=O)O)C4CC5CC(C4)CC3C5)nn1-c1ccc(-c3cnc4cc(C(=O)NCCCN(C)CCCNC(=O)CN5CCN(CC(=O)O)CCN(CC(=O)O)CCN(CC(=O)O)CC5)cnc4c3)cc1C(C)C)OCO2 ; COc1ccc2c(c1-c1cc(C(=O)NC3(C(=O)O)C4CC5CC(C4)CC3C5)nn1-c1ccc(OCCCN3CCN(C(=O)CN4CCN(CC(=O)O)CCN(CC(=O)O)CCN(CC(=O)O)CC4)CC3)cc1C(C)C)OCO2 ; COc1ccc2c(c1-c1cc(C(=O)NC3(C(=O)O)C4CC5CC(C4)CC3C5)nn1-c1ccc(OCCCN(C)CCCNC(=O)CN3CCN(CC(=O)O)CCN(CC(=O)O)CCN(CC(=O)O)CC3)nc1C(C)C)OCO2]

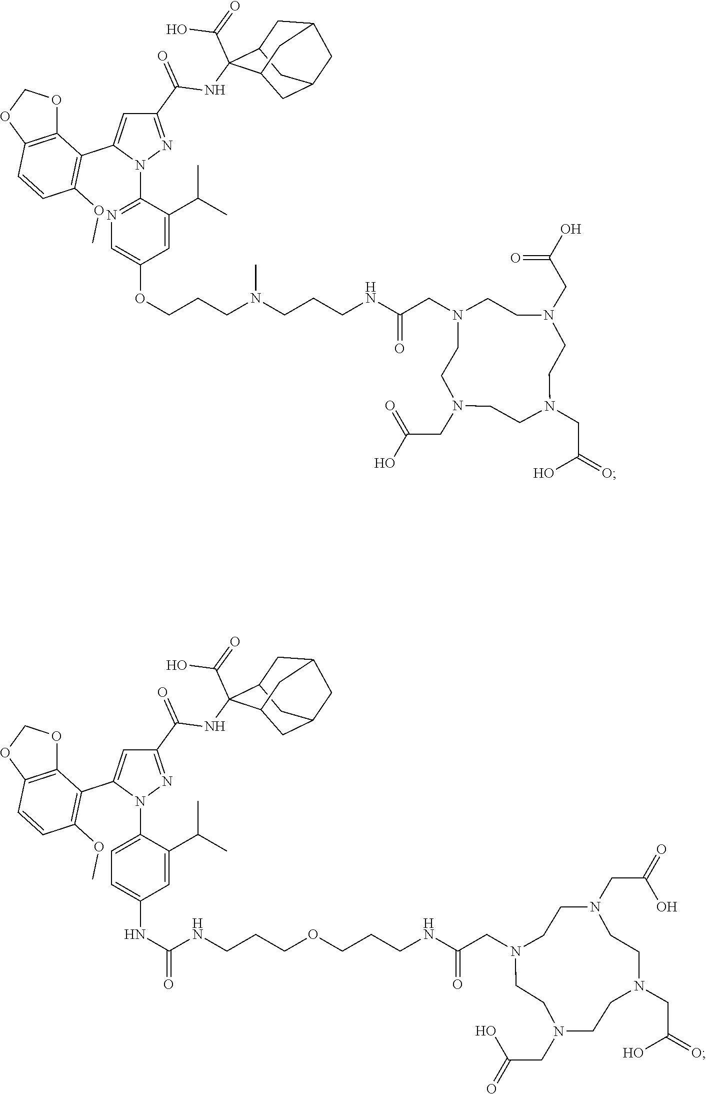 [SMILES: COc1ccc2c(c1-c1cc(C(=O)NC3(C(=O)O)C4CC5CC(C4)CC3C5)nn1-c1ncc(OCCCN(C)CCCNC(=O)CN3CCN(CC(=O)O)CCN(CC(=O)O)CCN(CC(=O)O)CC3)cc1C(C)C)OCO2 ; COc1ccc2c(c1-c1cc(C(=O)NC3(C(=O)O)C4CC5CC(C4)CC3C5)nn1-c1ccc(NC(=O)NCCCOCCCNC(=O)CN3CCN(CC(=O)O)CCN(CC(=O)O)CCN(CC(=O)O)CC3)cc1C(C)C)OCO2]

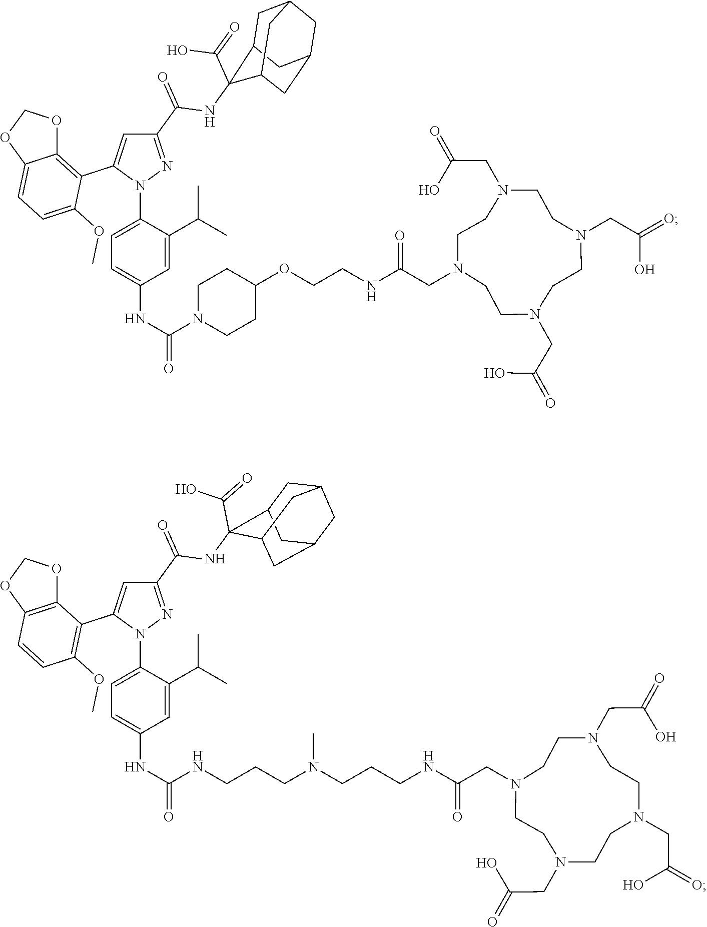 [SMILES: COc1ccc2c(c1-c1cc(C(=O)NC3(C(=O)O)C4CC5CC(C4)CC3C5)nn1-c1ccc(NC(=O)N3CCC(OCCNC(=O)CN4CCN(CC(=O)O)CCN(CC(=O)O)CCN(CC(=O)O)CC4)CC3)cc1C(C)C)OCO2 ; COc1ccc2c(c1-c1cc(C(=O)NC3(C(=O)O)C4CC5CC(C4)CC3C5)nn1-c1ccc(NC(=O)NCCCN(C)CCCNC(=O)CN3CCN(CC(=O)O)CCN(CC(=O)O)CCN(CC(=O)O)CC3)cc1C(C)C)OCO2]

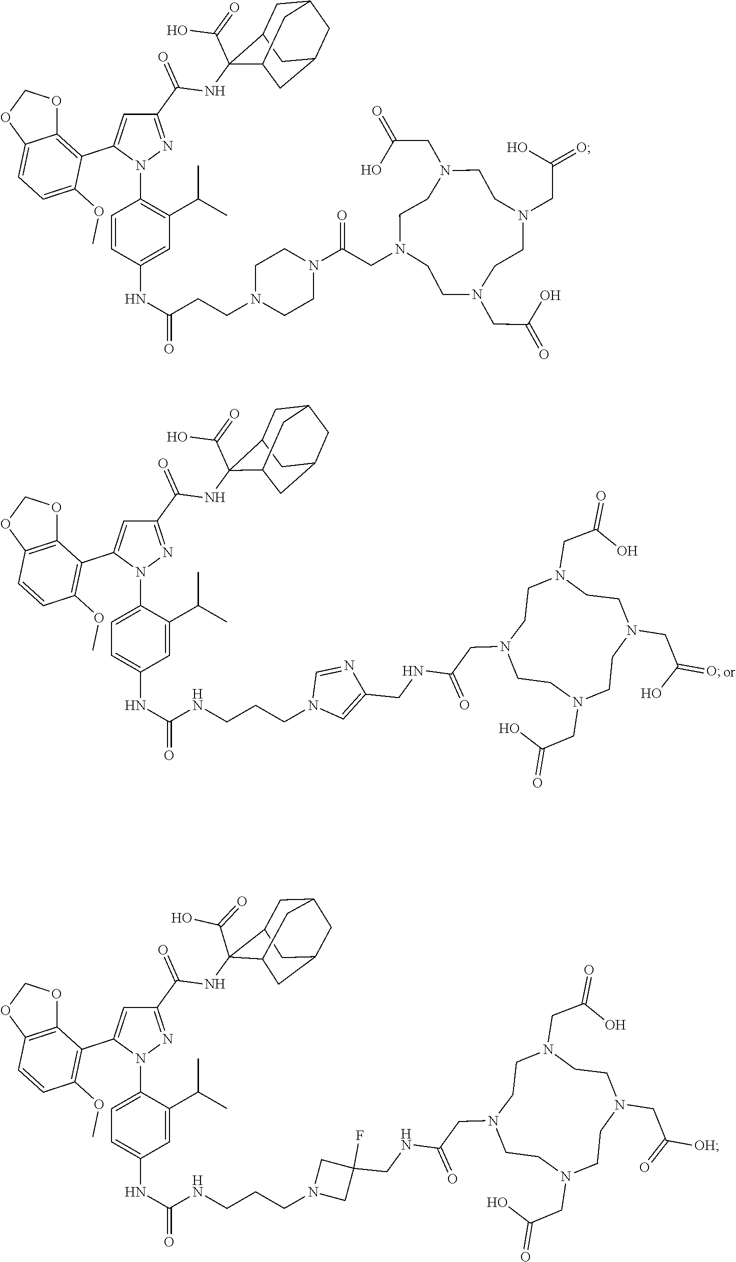 [SMILES: COc1ccc2c(c1-c1cc(C(=O)NC3(C(=O)O)C4CC5CC(C4)CC3C5)nn1-c1ccc(NC(=O)CCN3CCN(C(=O)CN4CCN(CC(=O)O)CCN(CC(=O)O)CCN(CC(=O)O)CC4)CC3)cc1C(C)C)OCO2 ; COc1ccc2c(c1-c1cc(C(=O)NC3(C(=O)O)C4CC5CC(C4)CC3C5)nn1-c1ccc(NC(=O)NCCCn3cnc(CNC(=O)CN4CCN(CC(=O)O)CCN(CC(=O)O)CCN(CC(=O)O)CC4)c3)cc1C(C)C)OCO2 ; COc1ccc2c(c1-c1cc(C(=O)NC3(C(=O)O)C4CC5CC(C4)CC3C5)nn1-c1ccc(NC(=O)NCCCN3CC(F)(CNC(=O)CN4CCN(CC(=O)O)CCN(CC(=O)O)CCN(CC(=O)O)CC4)C3)cc1C(C)C)OCO2]

or a pharmaceutically acceptable salt thereof.

The present invention also provides a compound that is

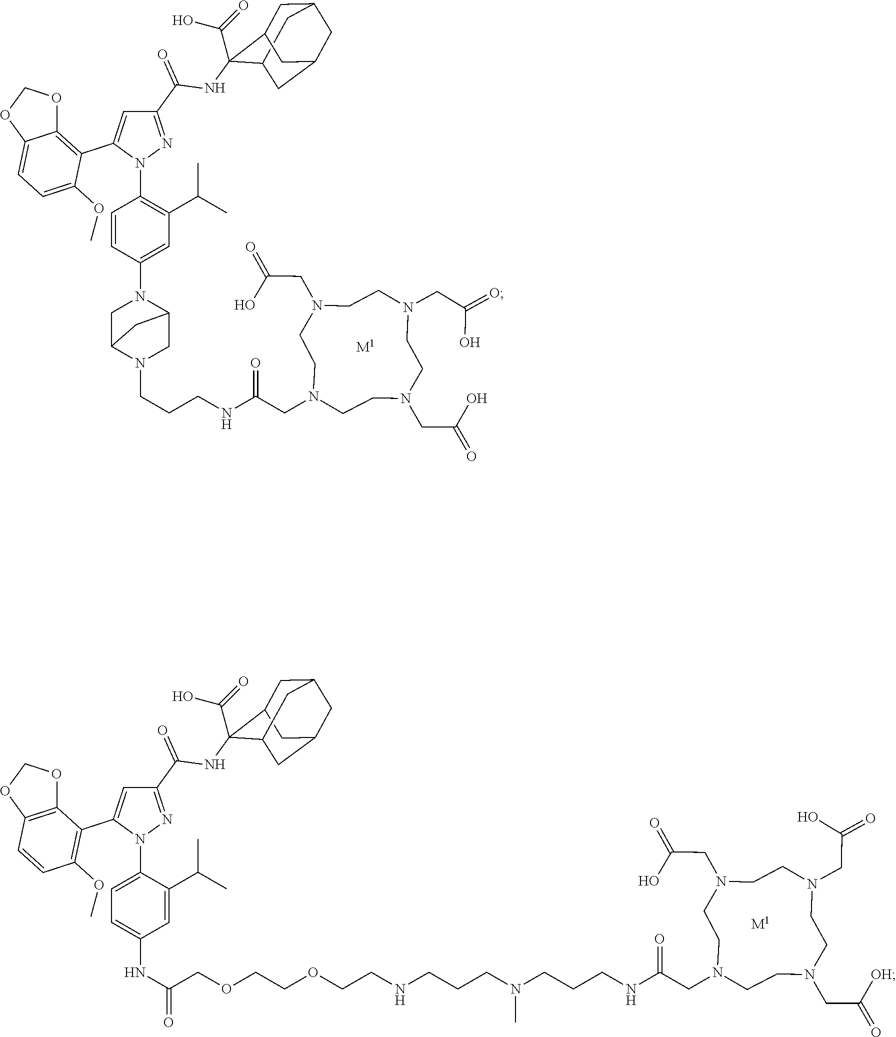

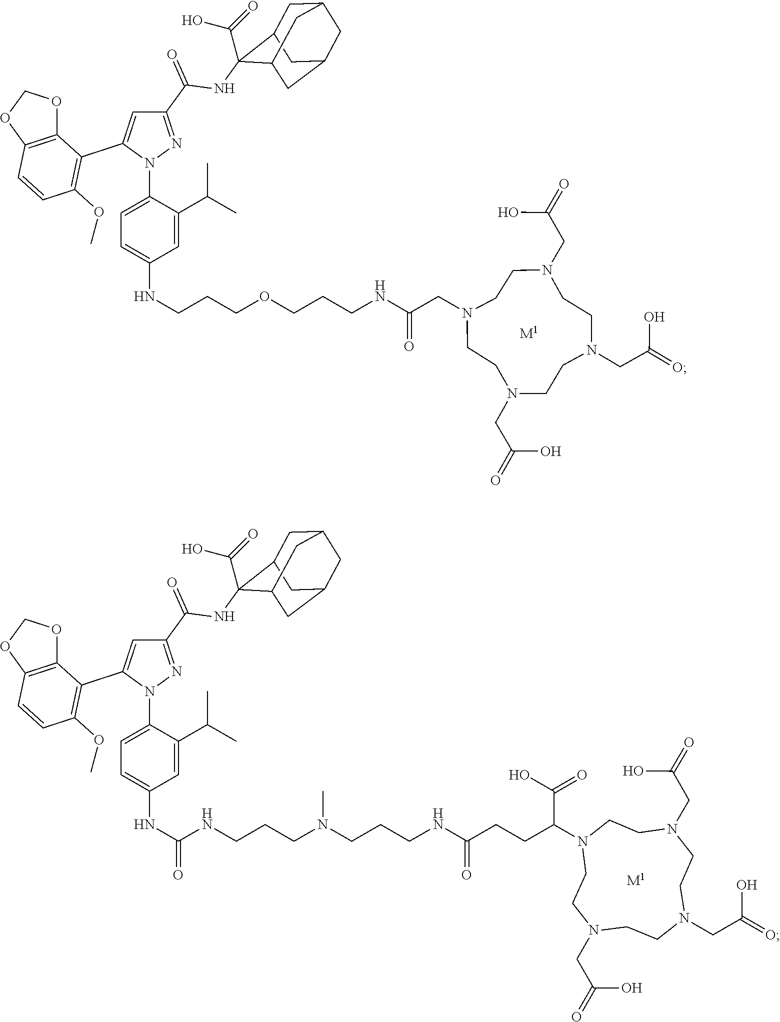

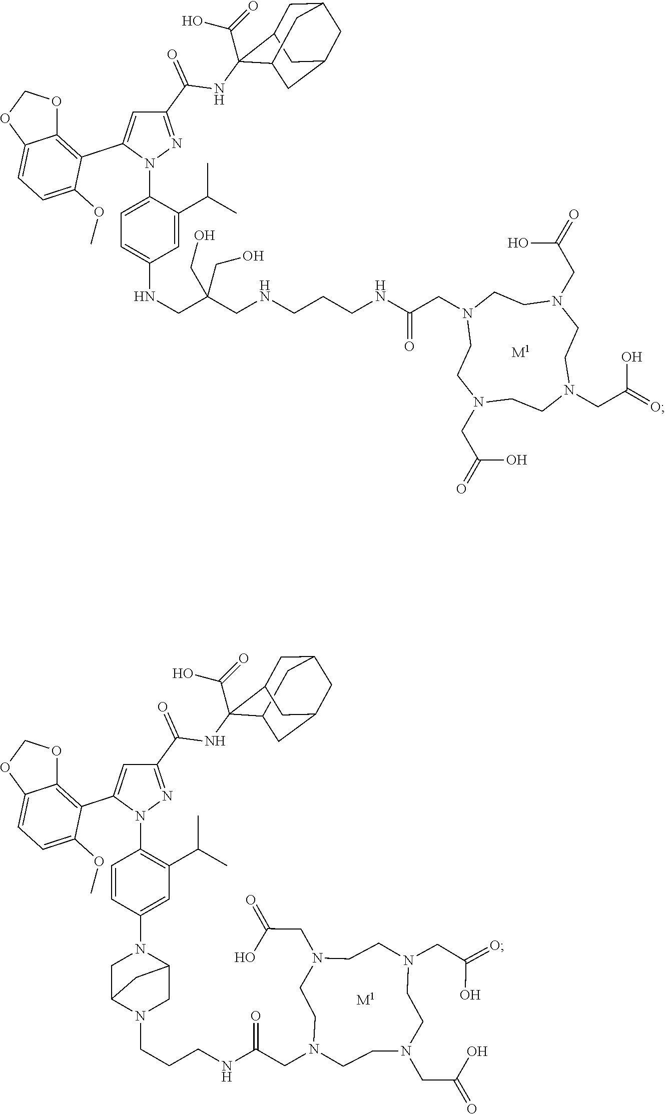

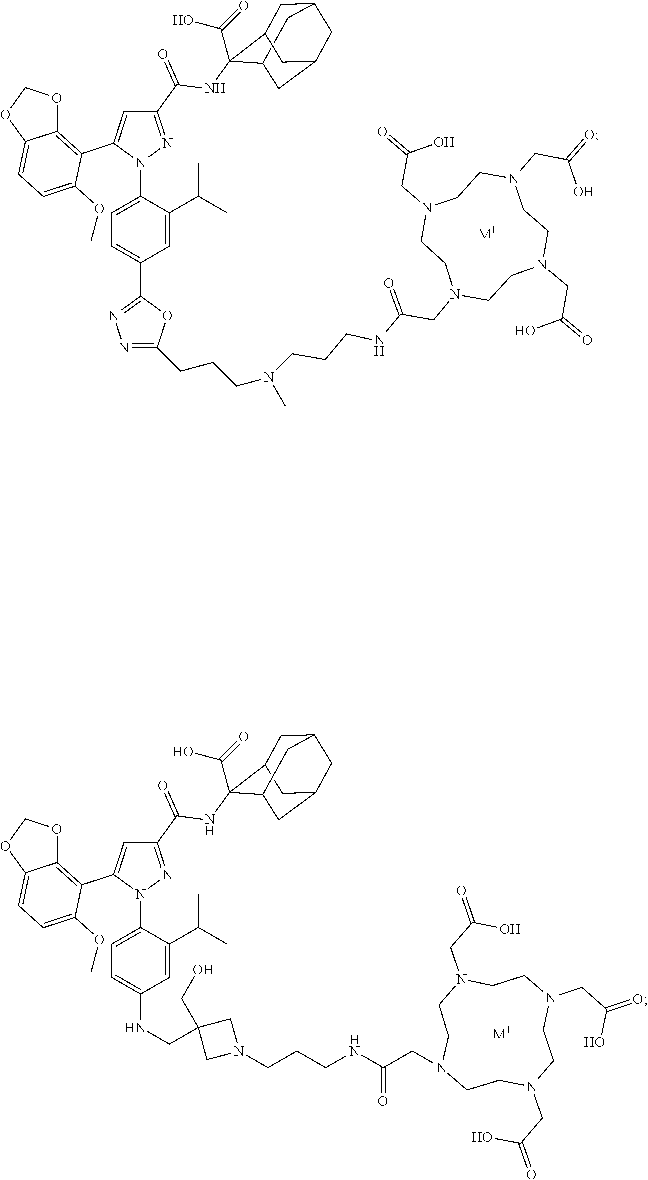

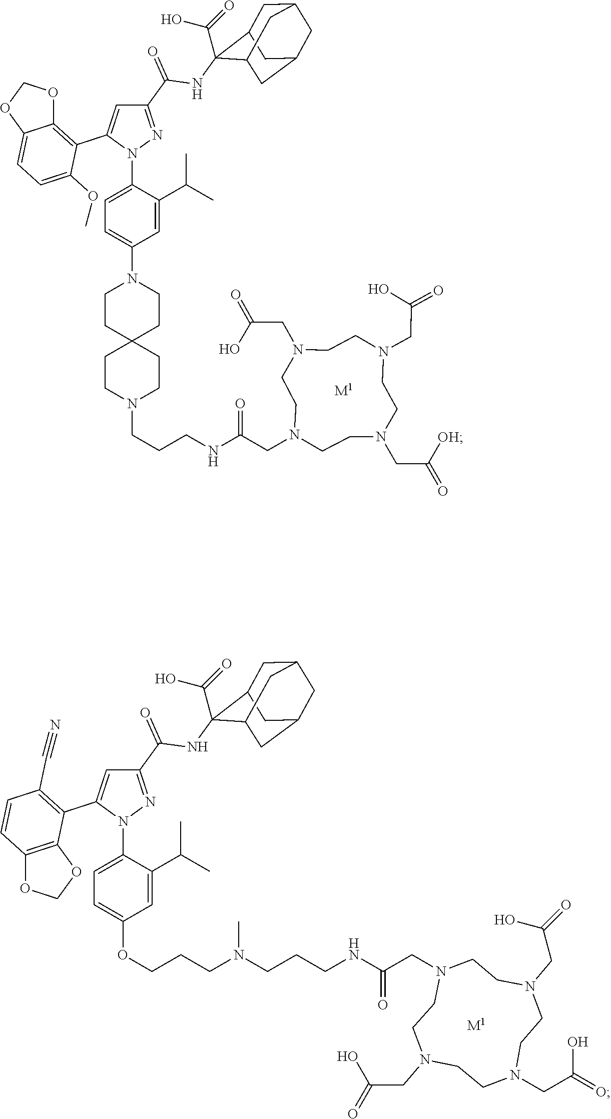

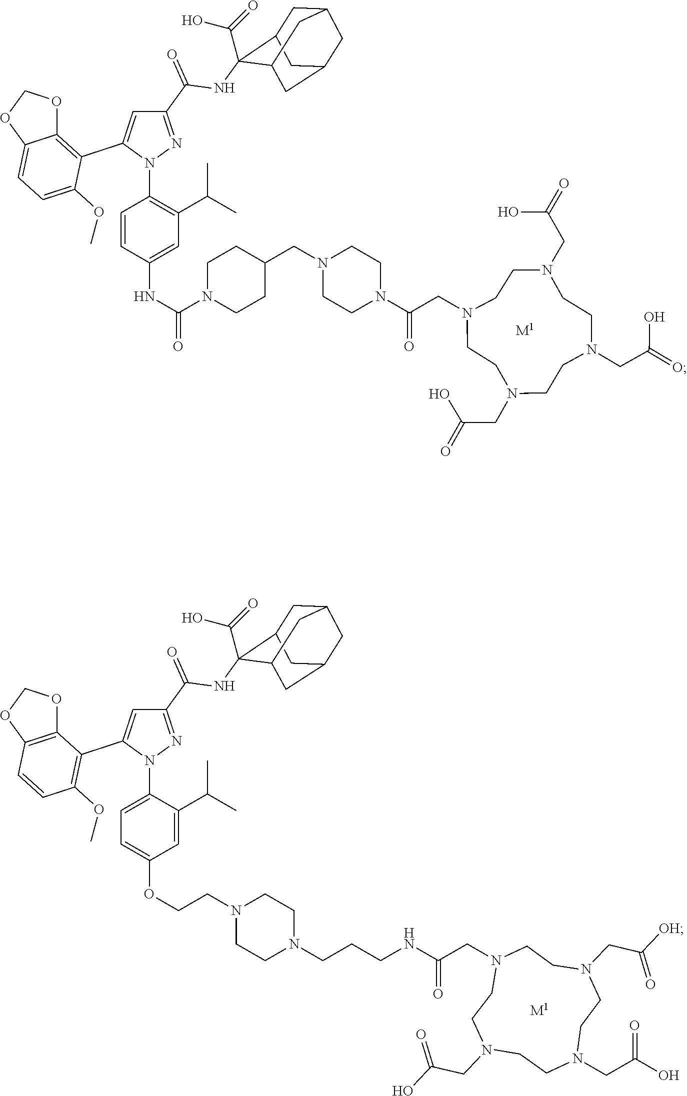

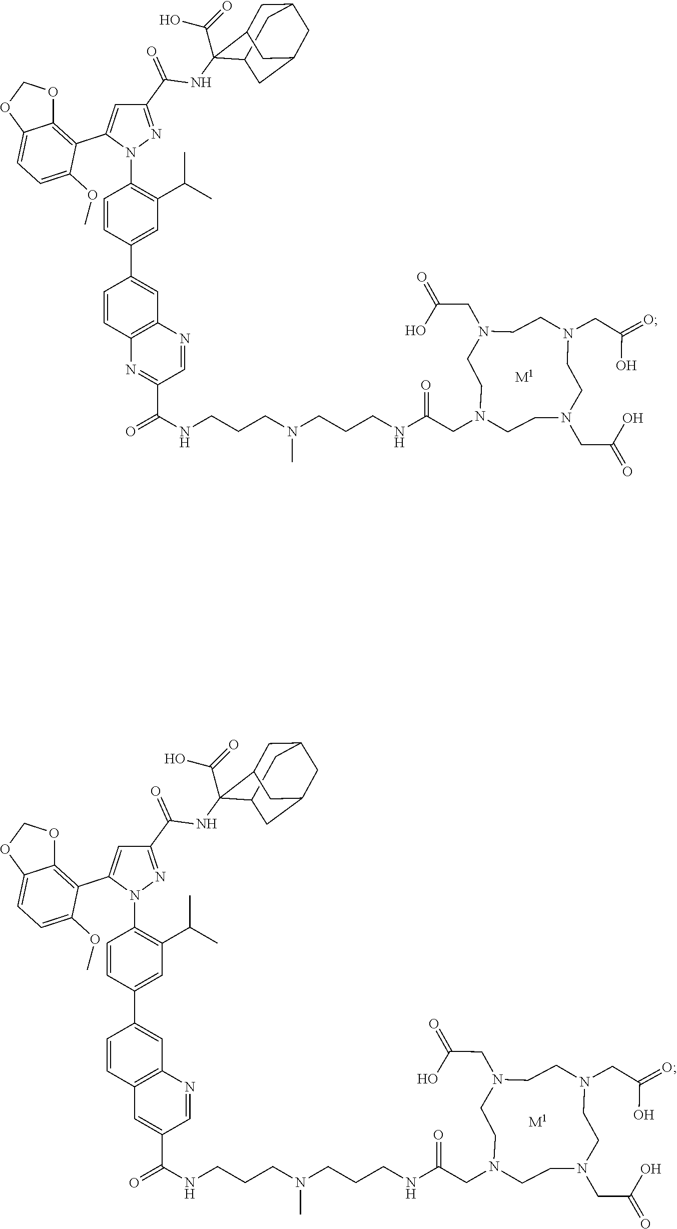

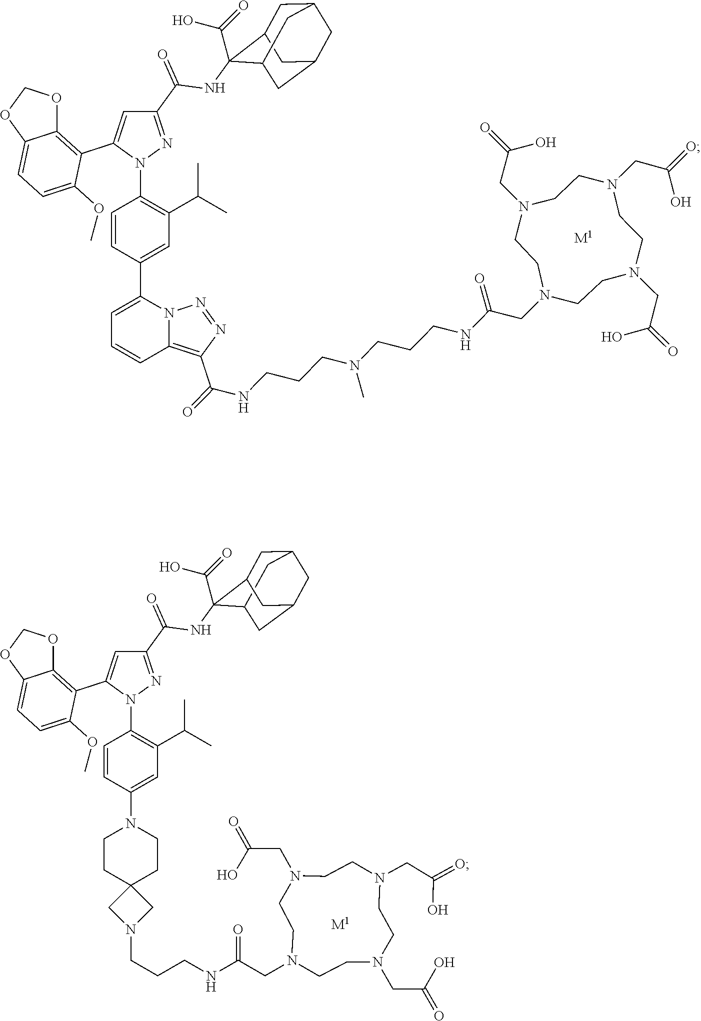

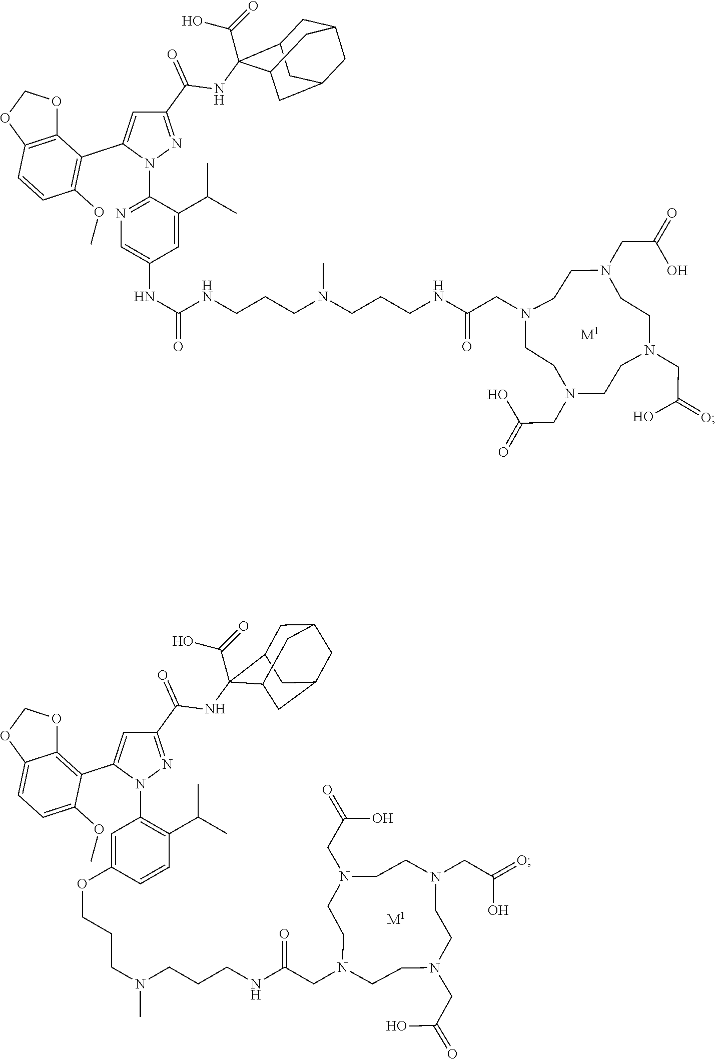

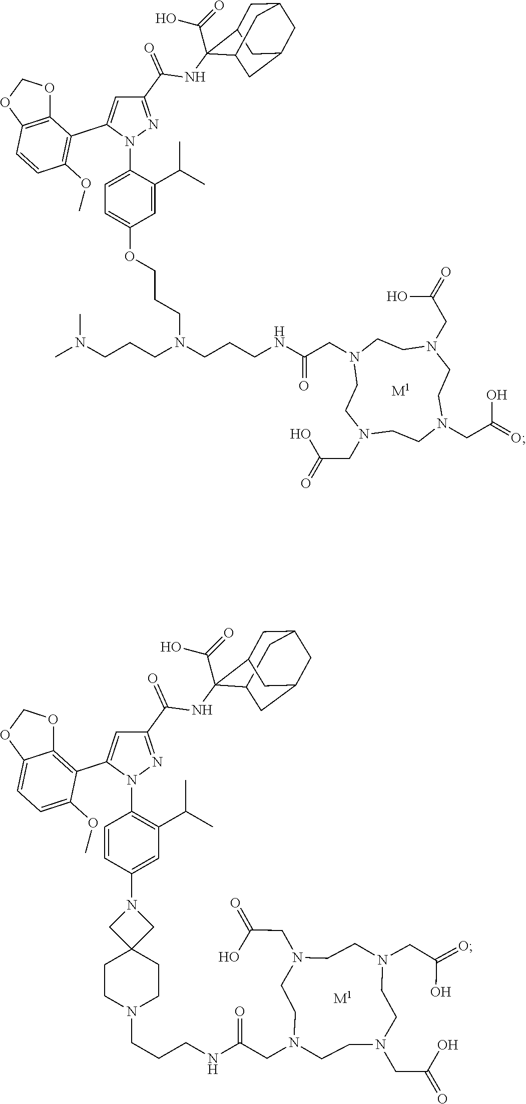

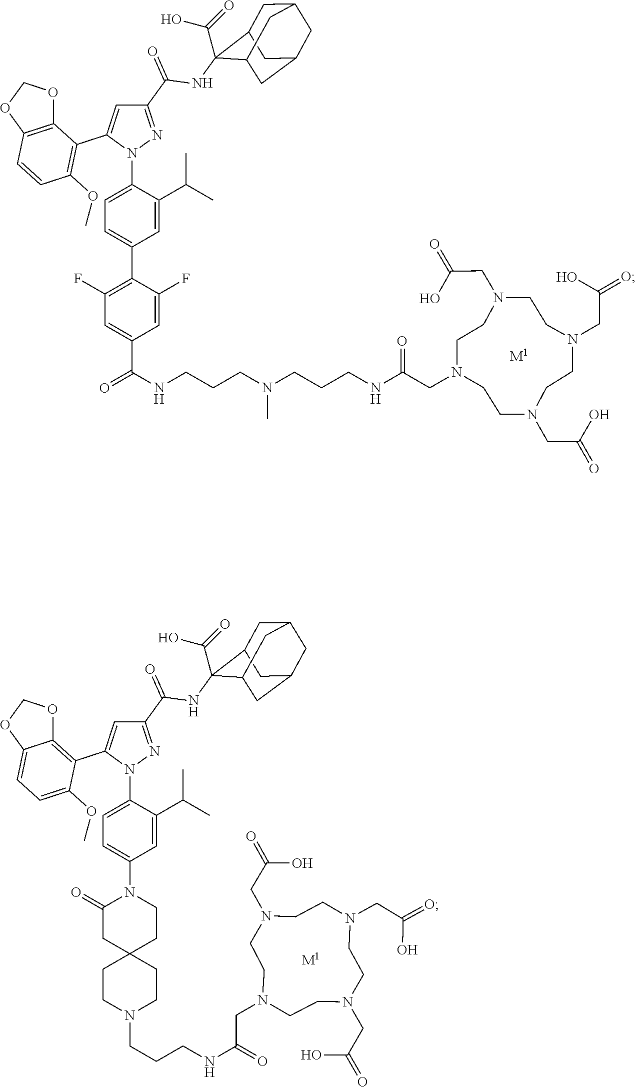

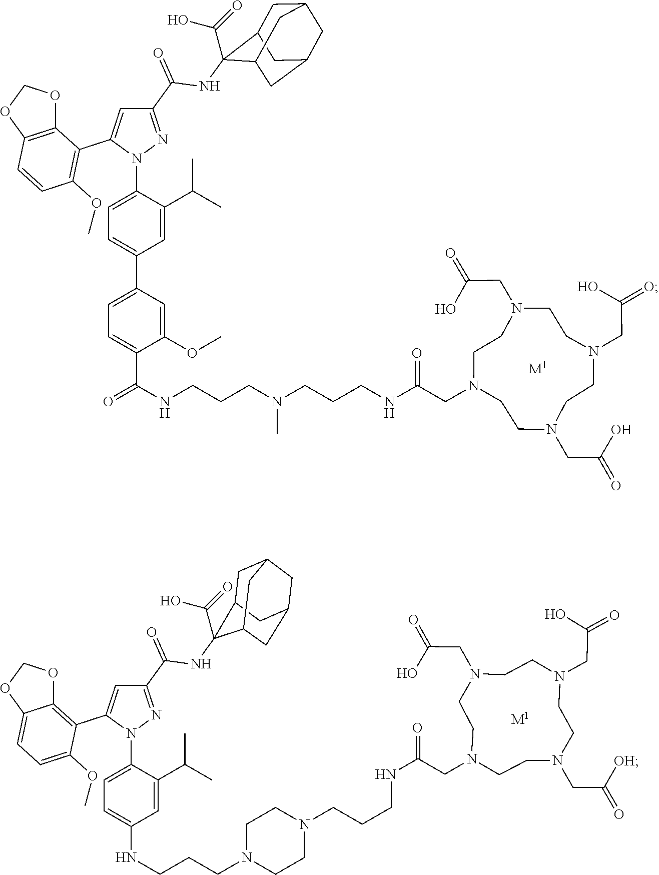

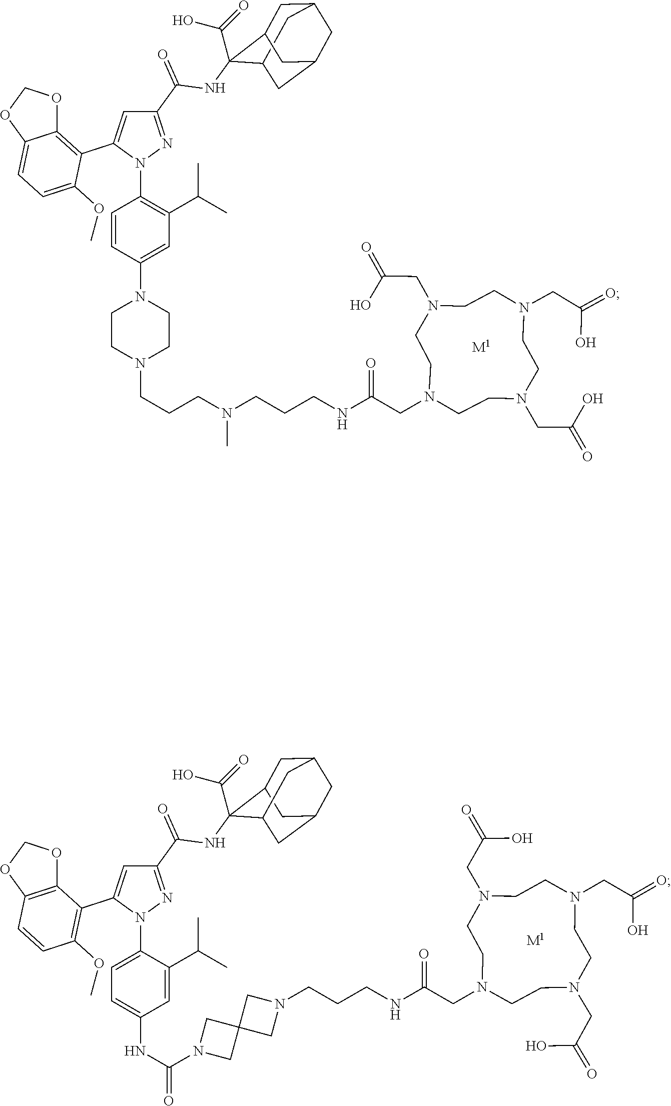

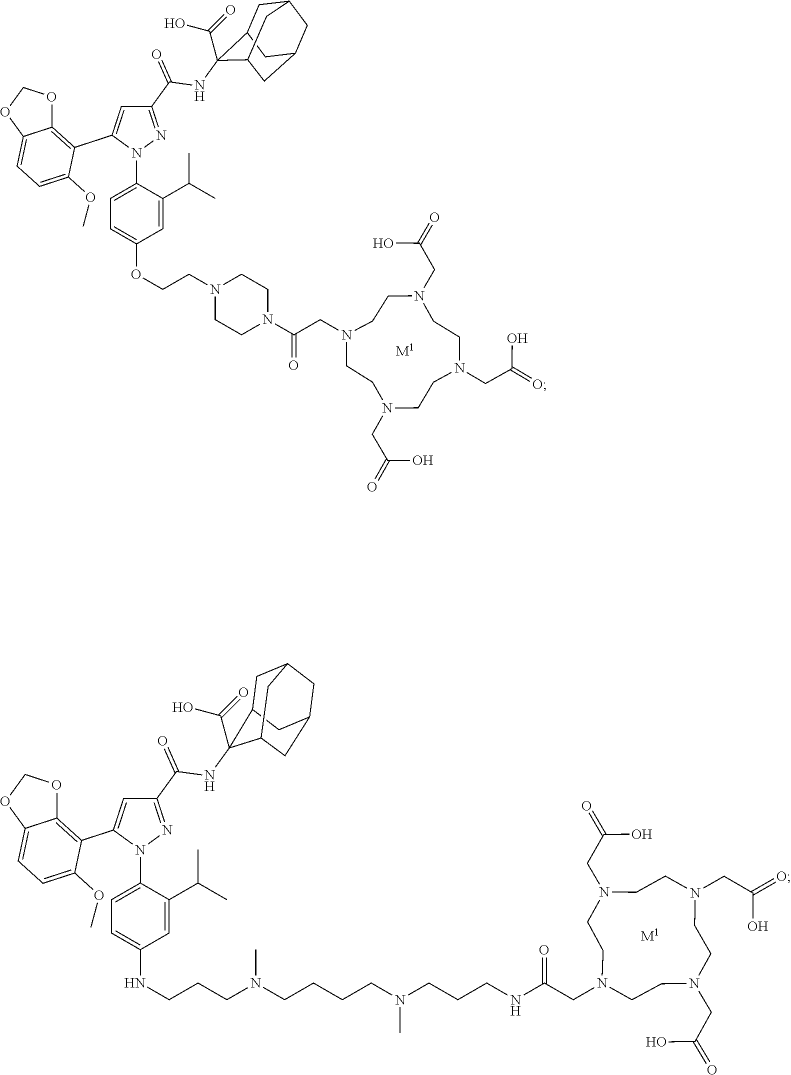

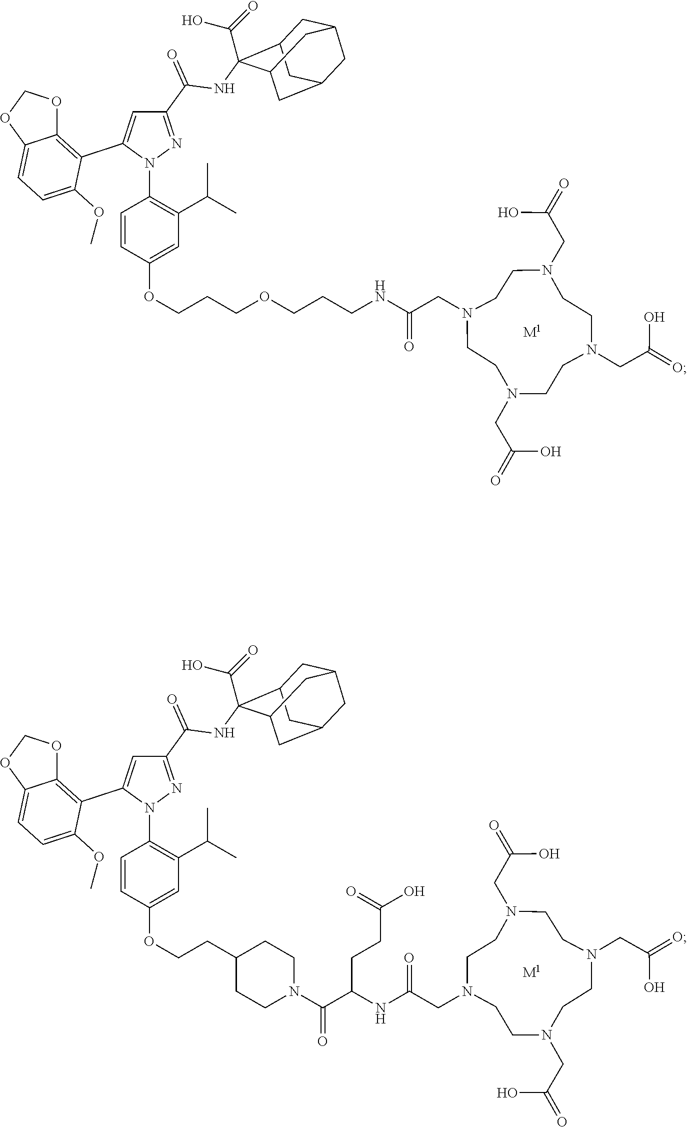 [SMILES: COc1ccc2c(c1-c1cc(C(=O)NC3(C(=O)O)C4CC5CC(C4)CC3C5)nn1-c1ccc(OCCCOCCCNC(=O)CN3CCN(CC(=O)O)CCN(CC(=O)O)CCN(CC(=O)O)CC3)cc1C(C)C)OCO2 ; "M1" ; COc1ccc2c(c1-c1cc(C(=O)NC3(C(=O)O)C4CC5CC(C4)CC3C5)nn1-c1ccc(OCCC3CCN(C(=O)C(CCC(=O)O)NC(=O)CN4CCN(CC(=O)O)CCN(CC(=O)O)CCN(CC(=O)O)CC4)CC3)cc1C(C)C)OCO2 ; "M1"]

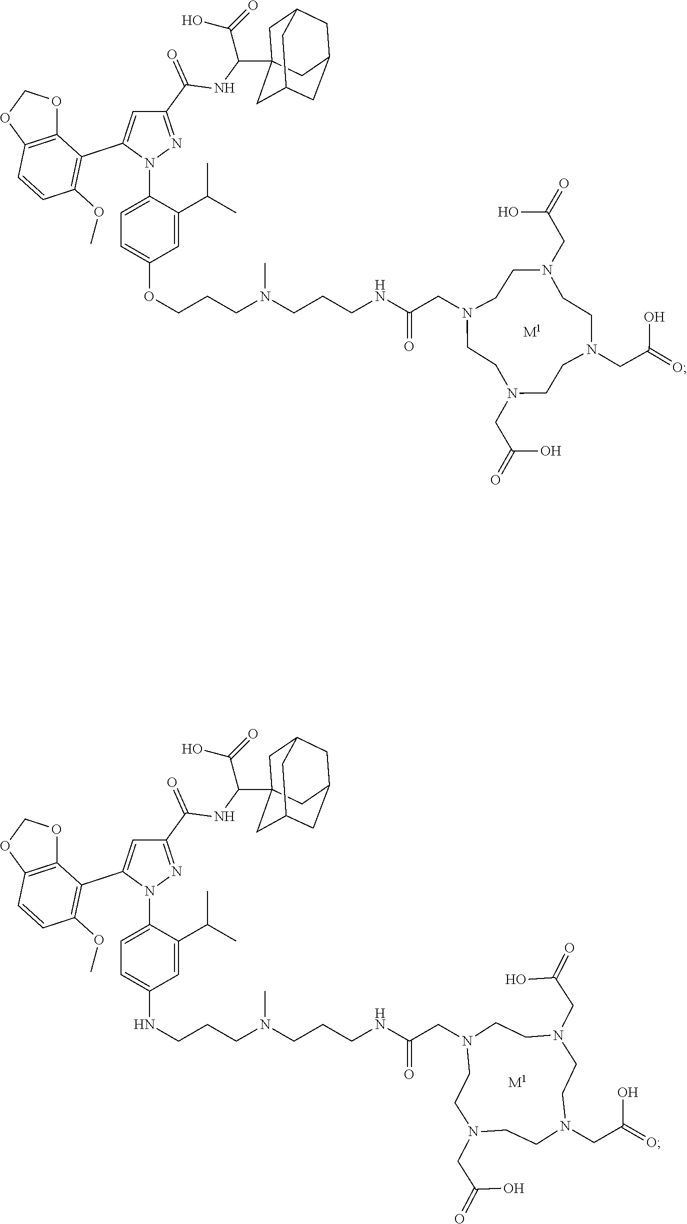

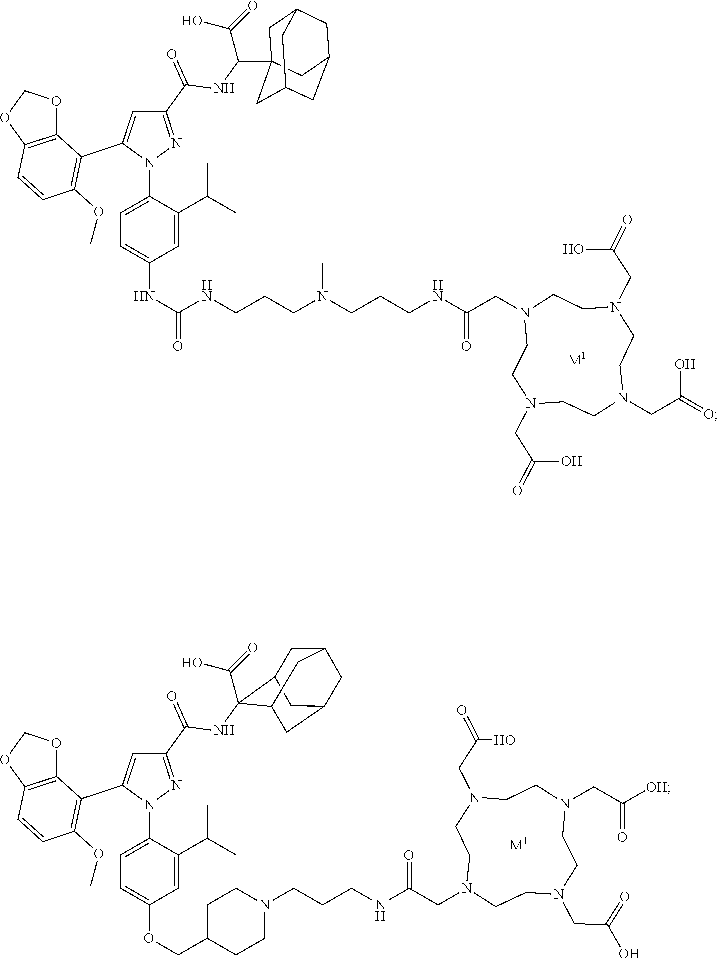

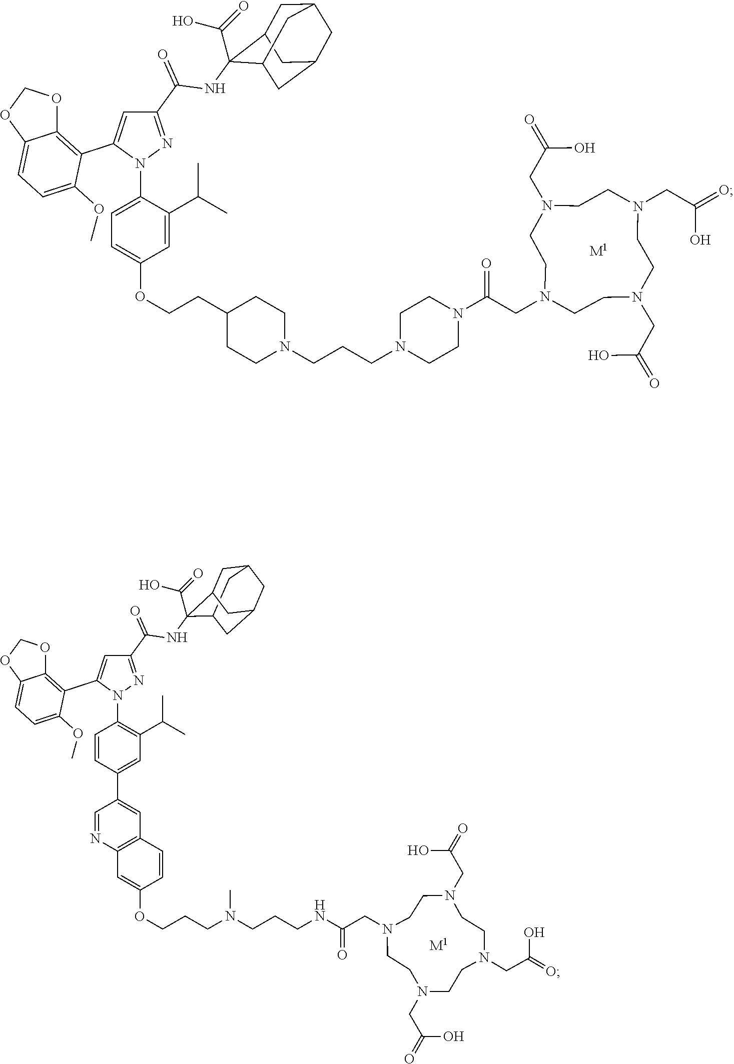

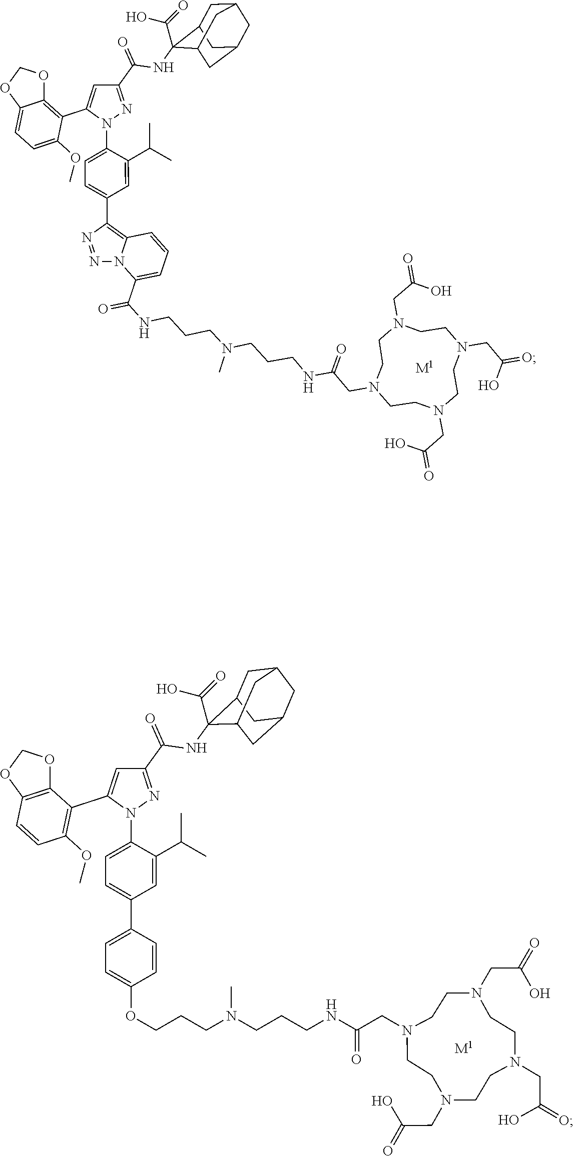

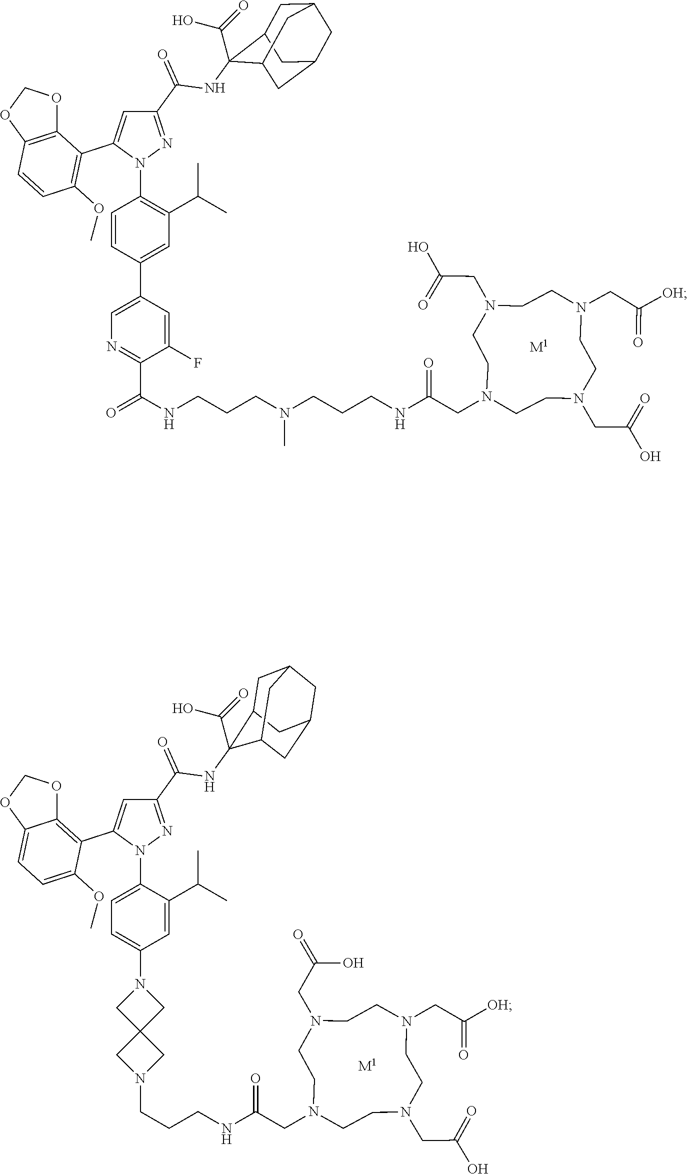

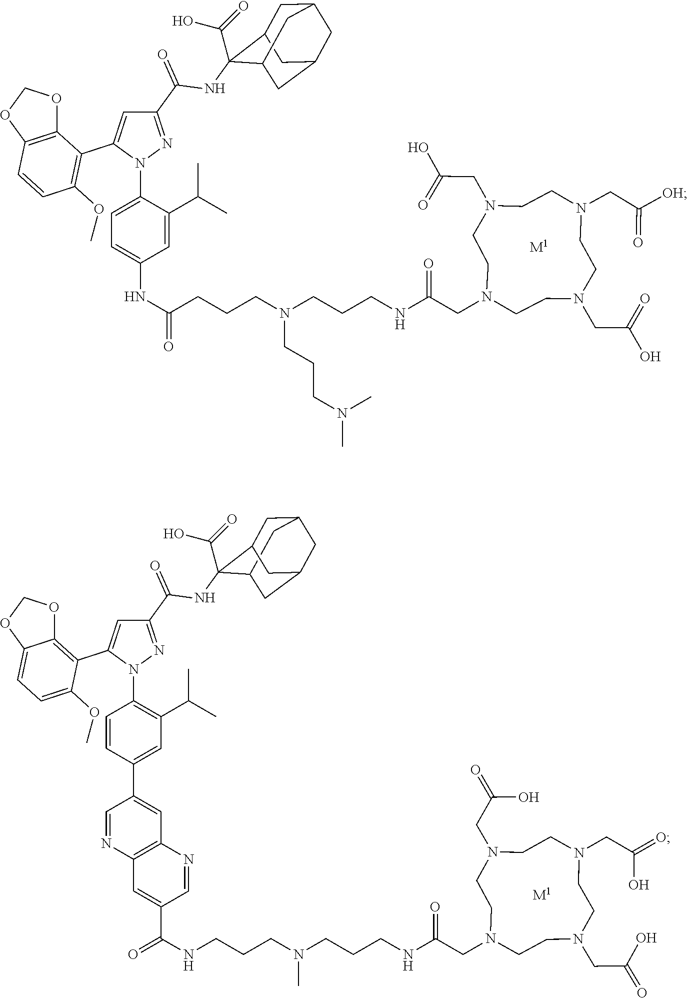

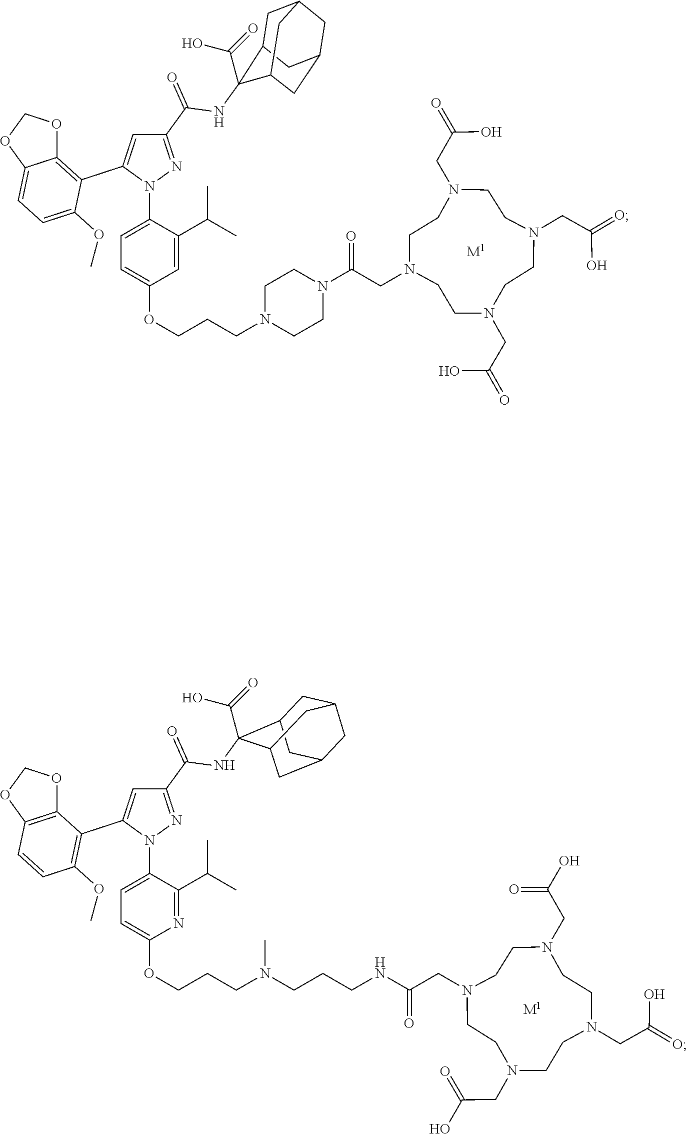

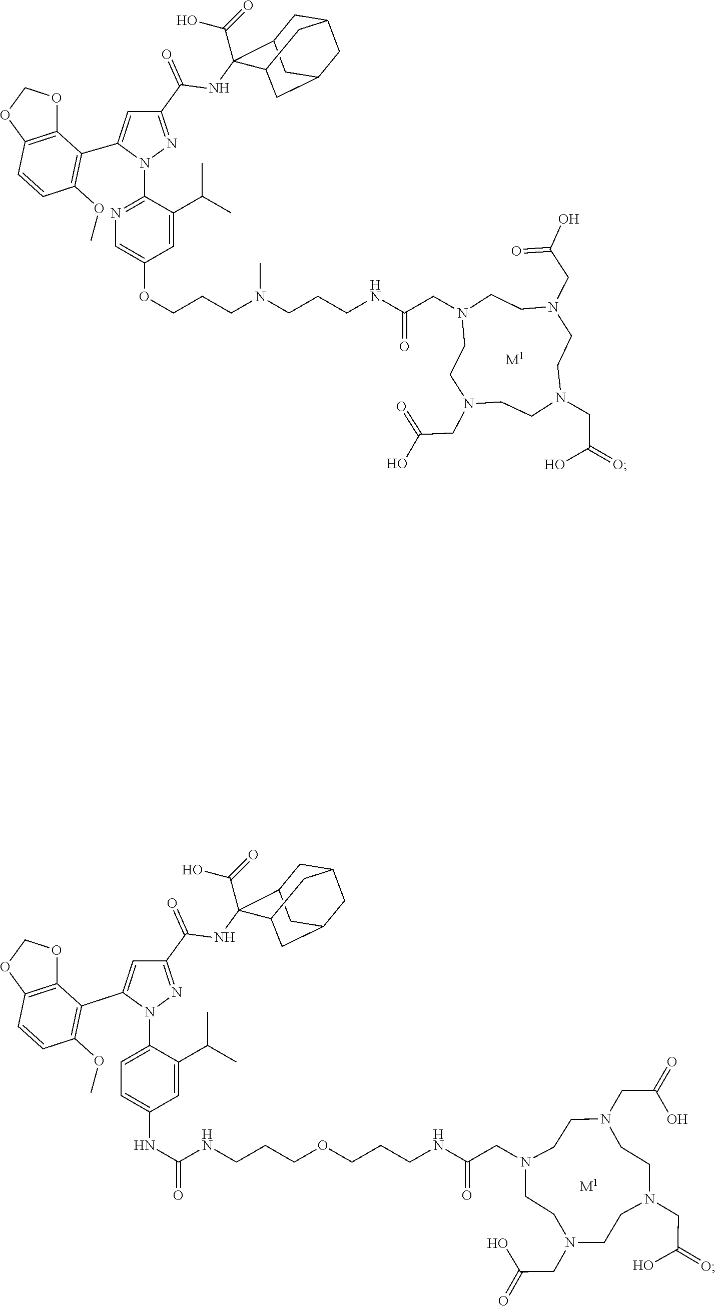

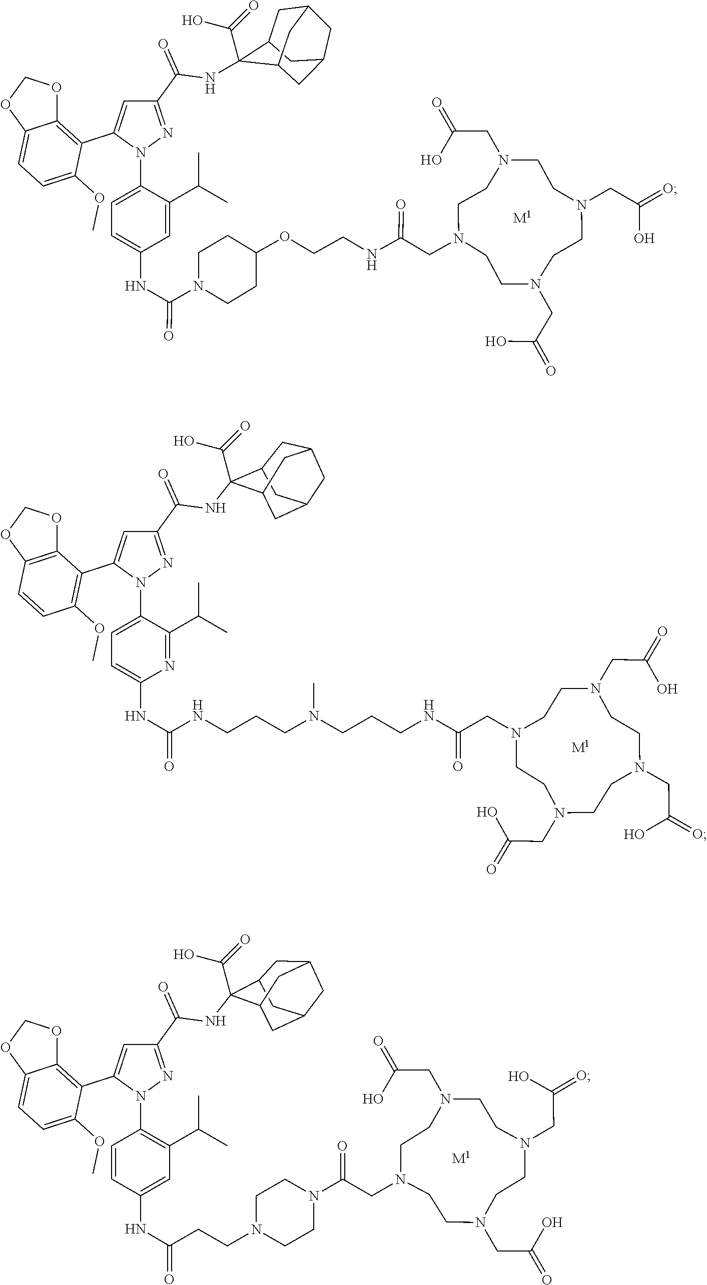

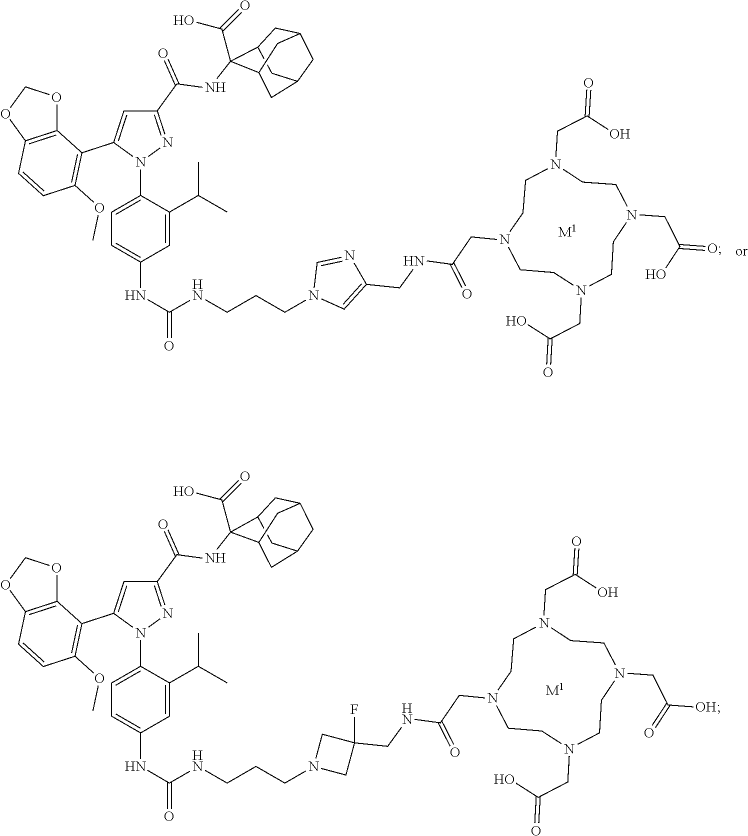

or a pharmaceutically acceptable salt thereof. The present invention also provides a compound that is

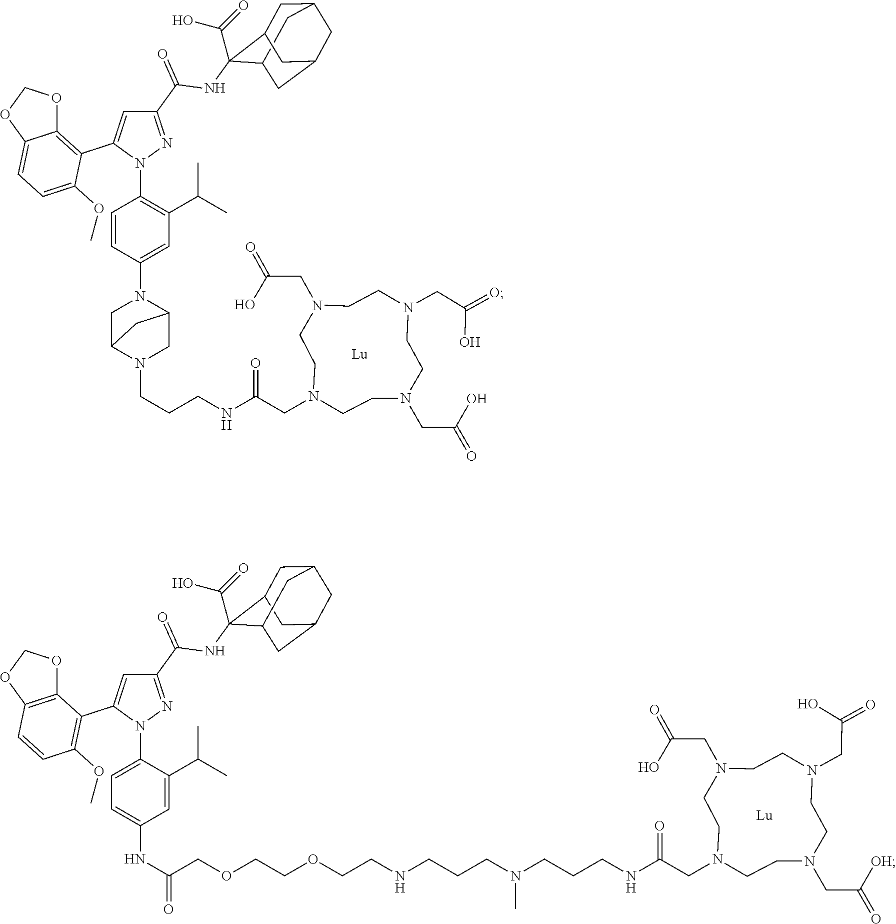

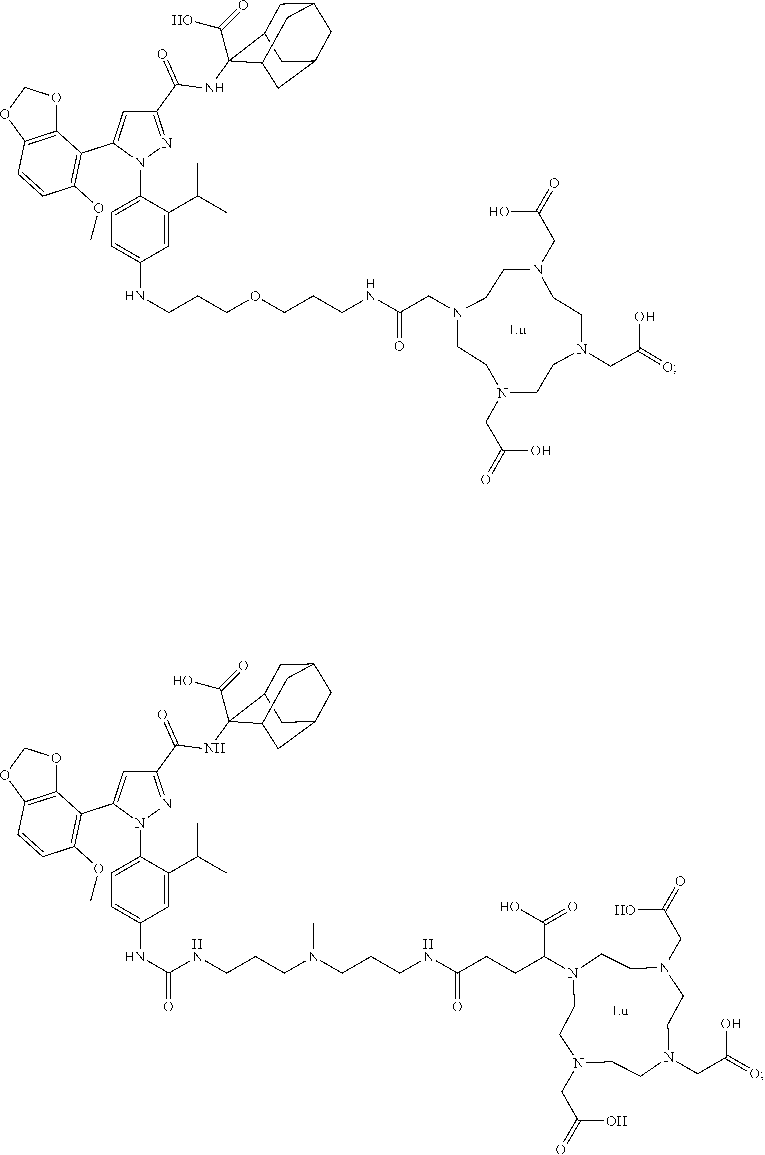

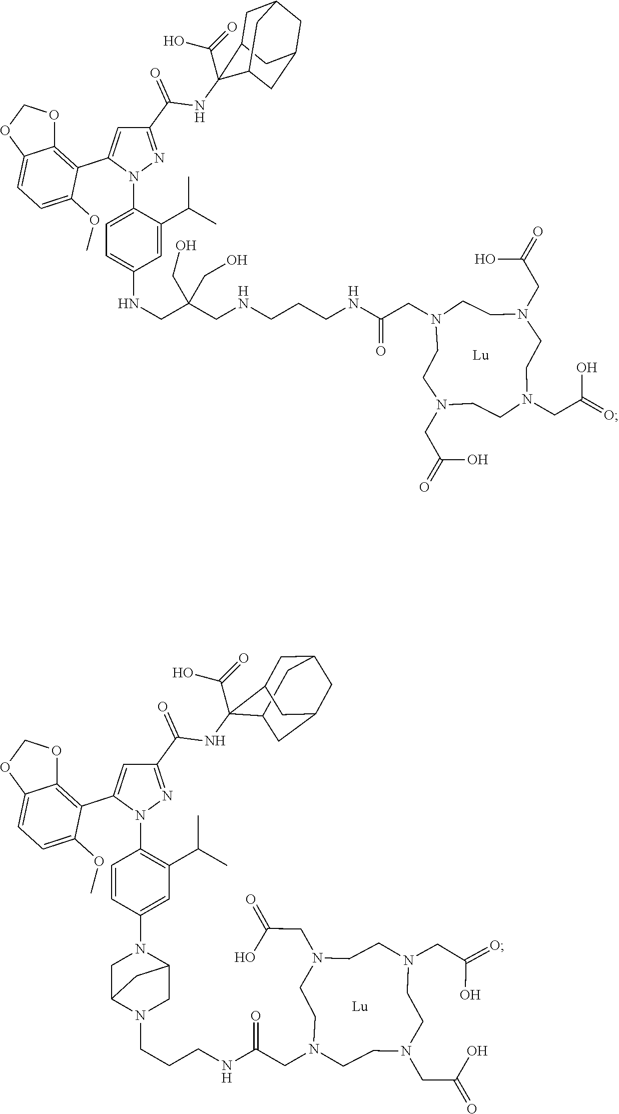

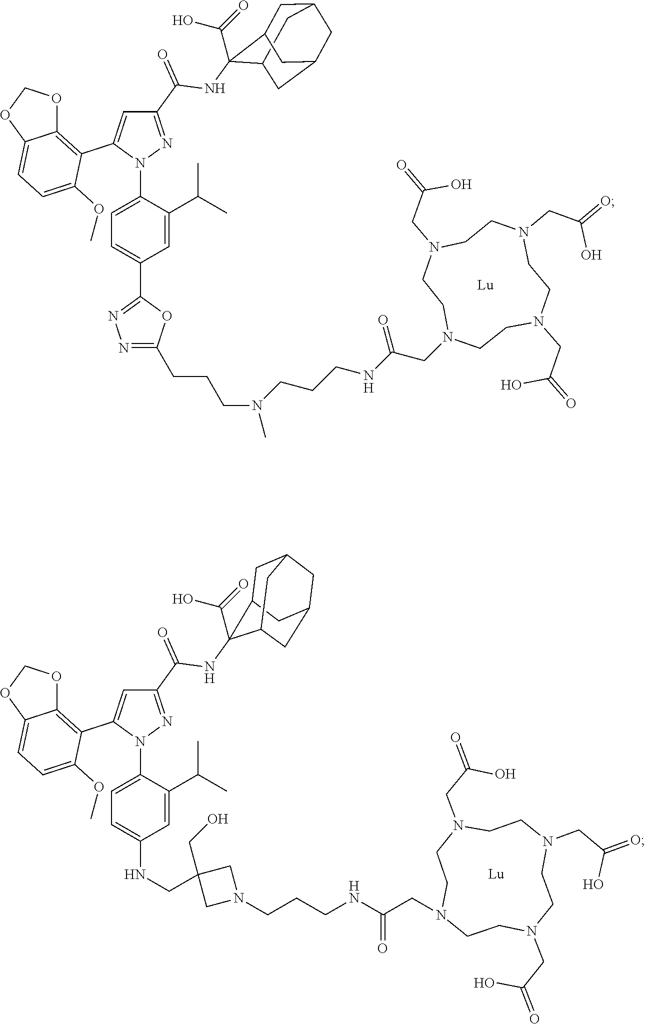

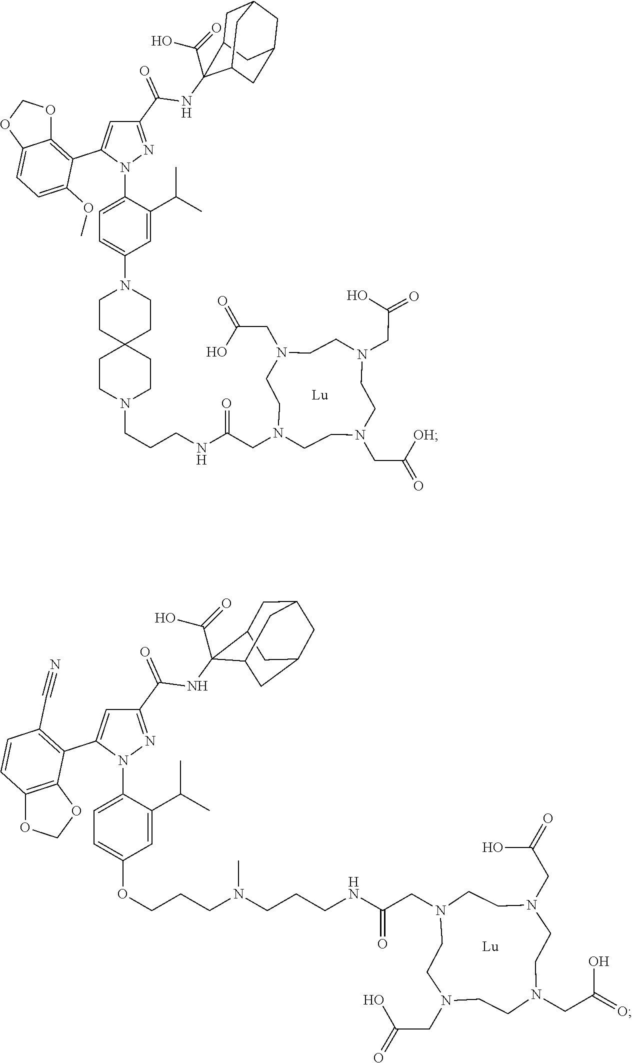

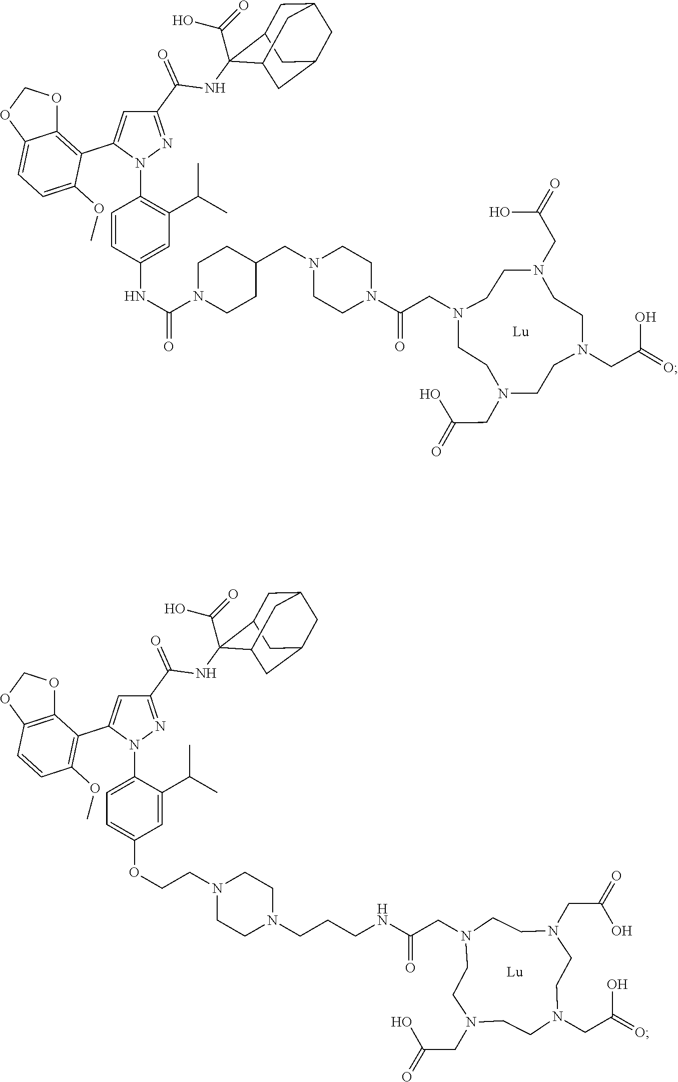

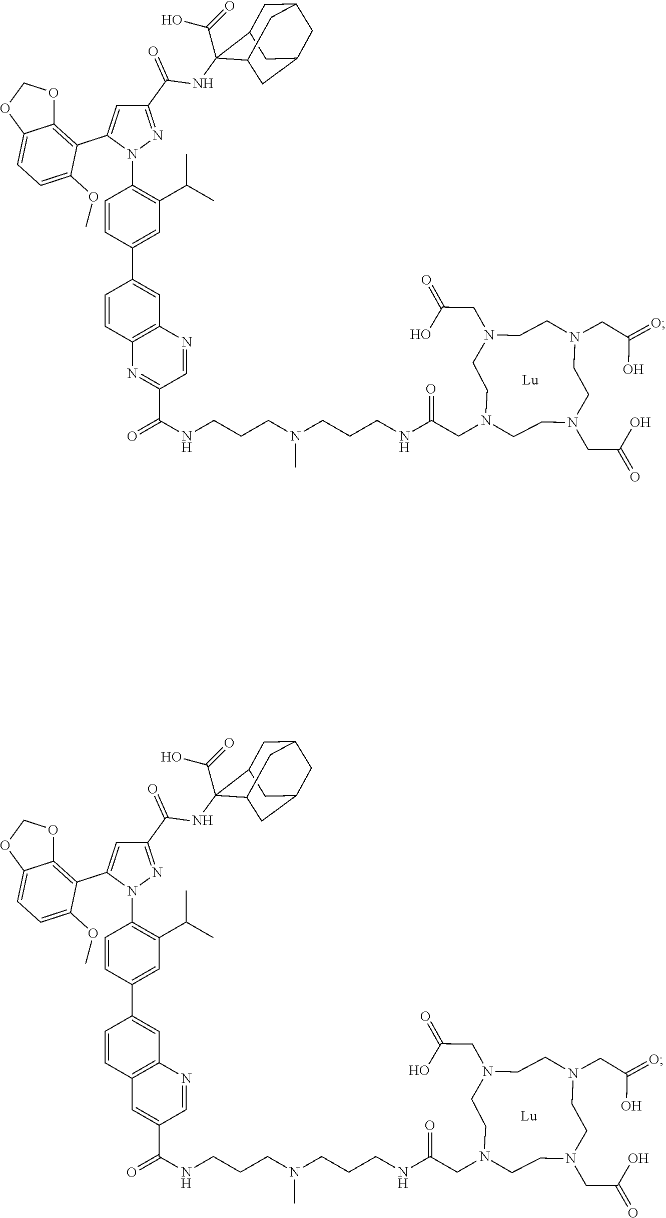

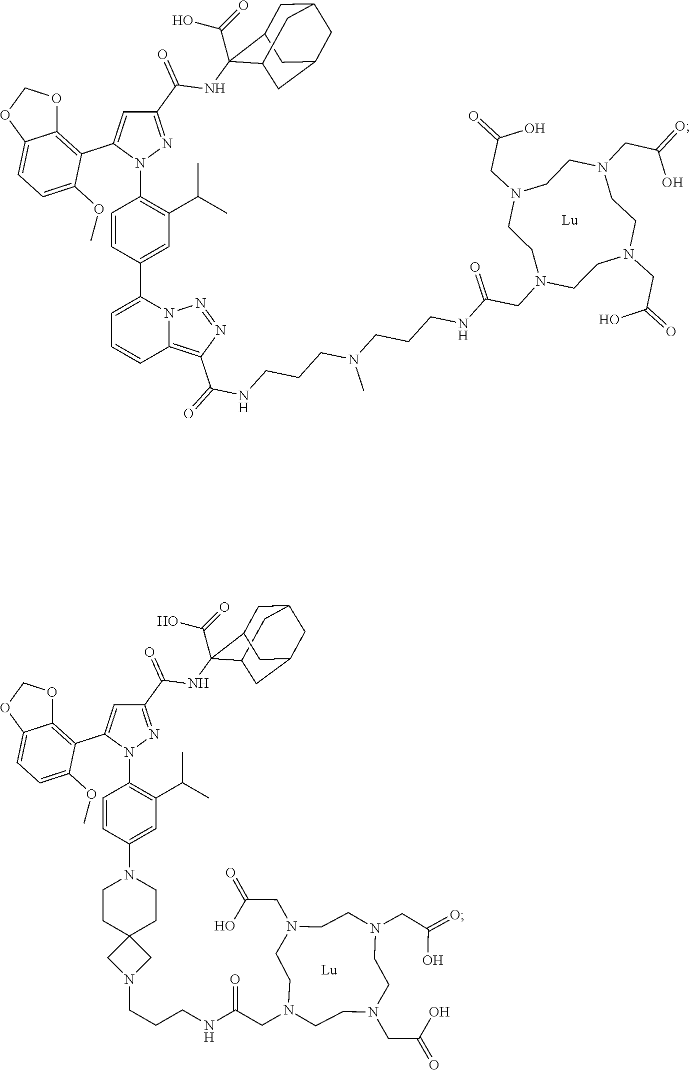

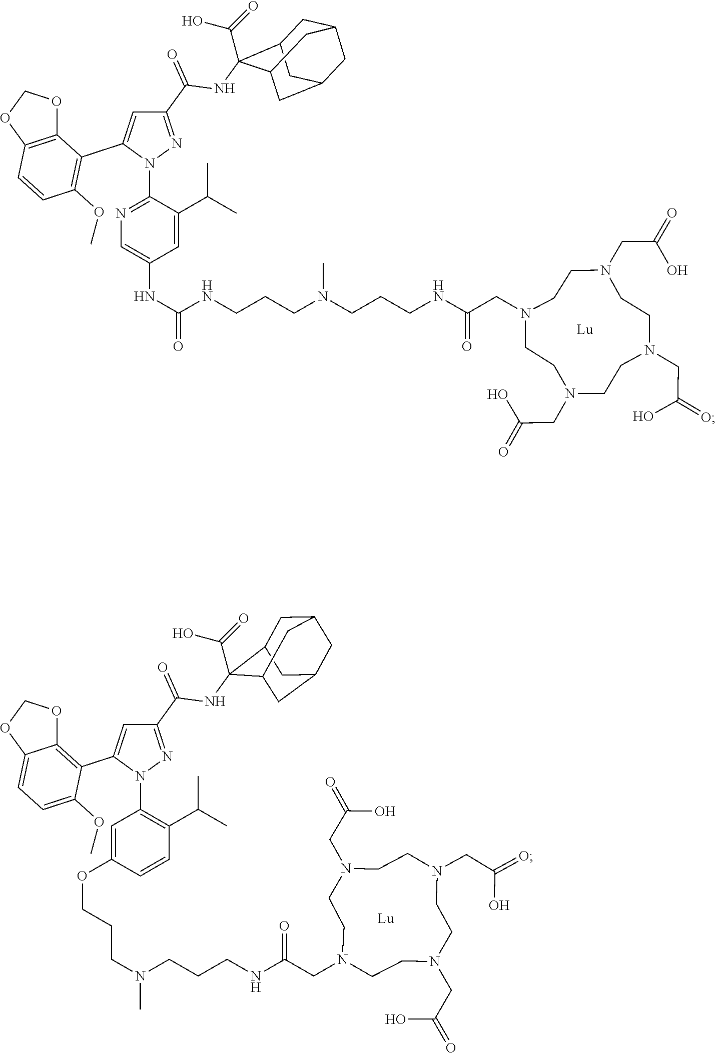

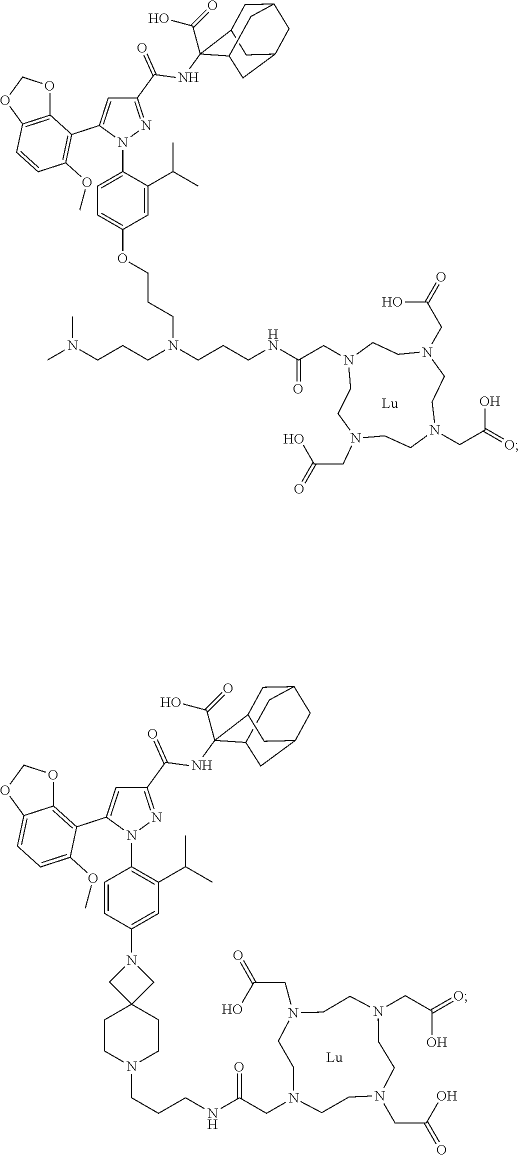

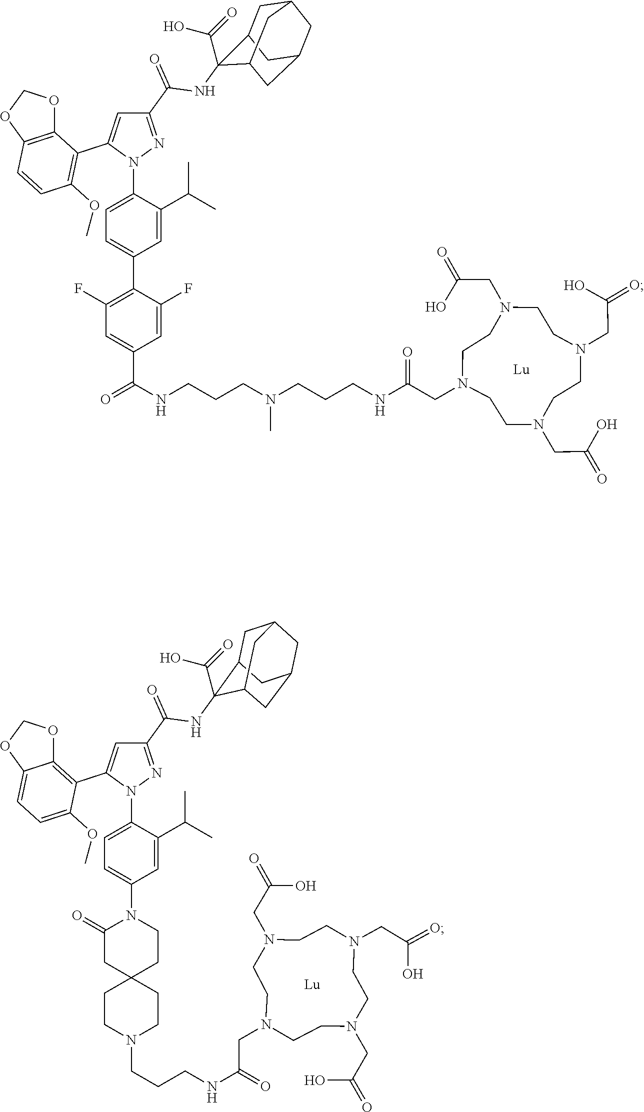

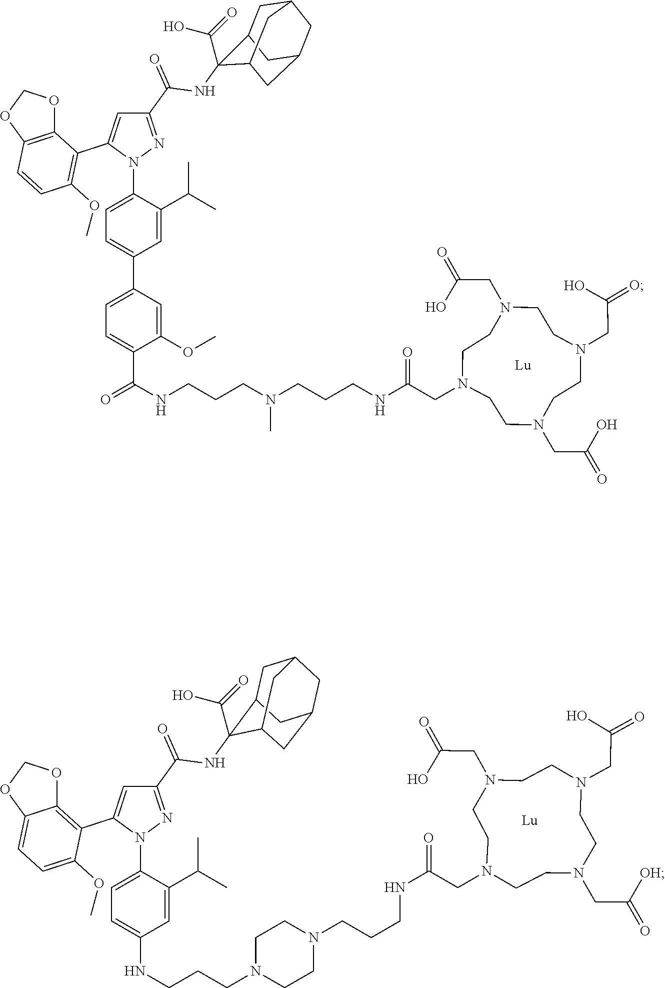

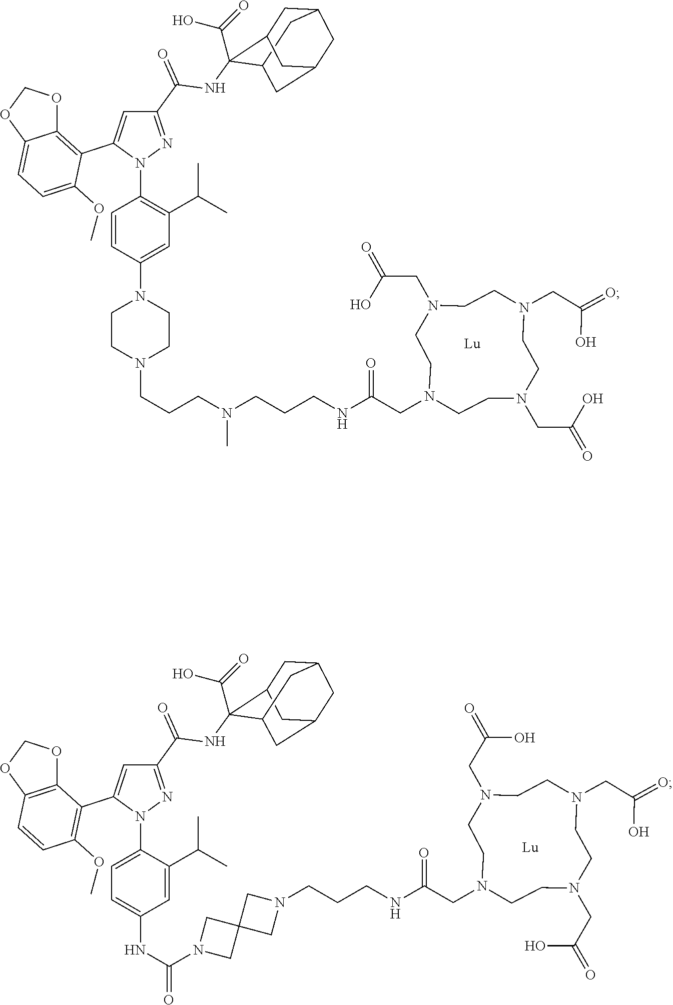

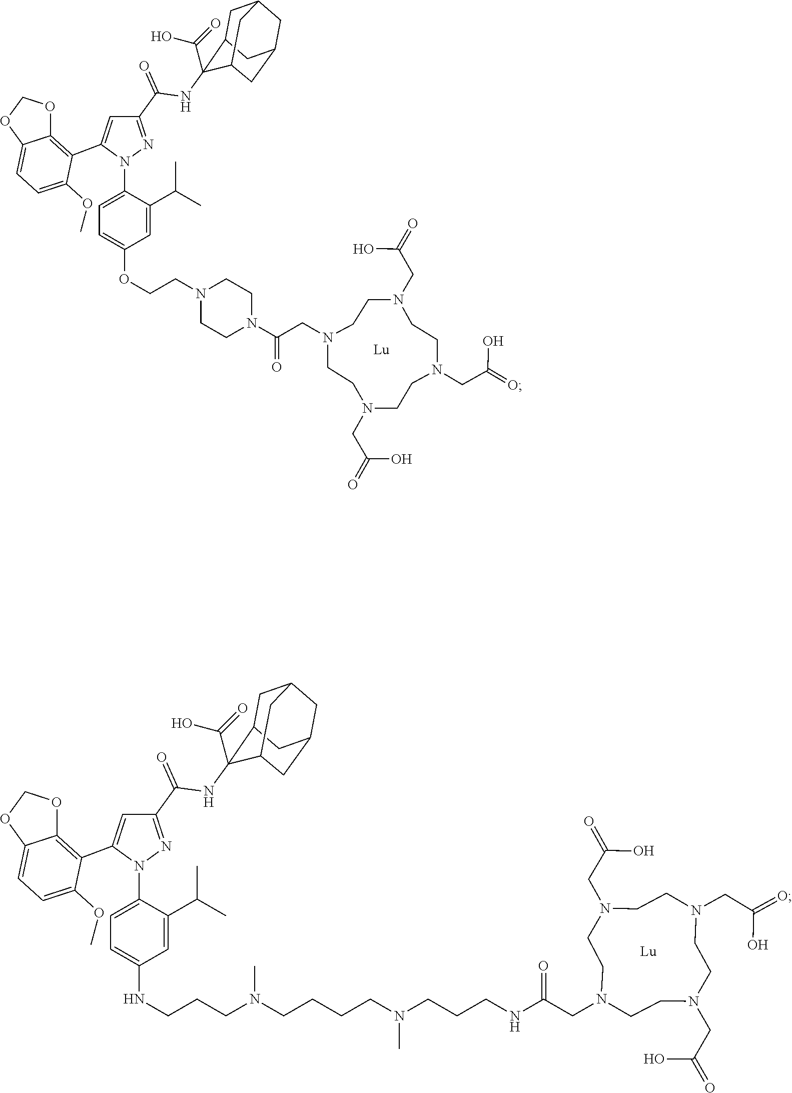

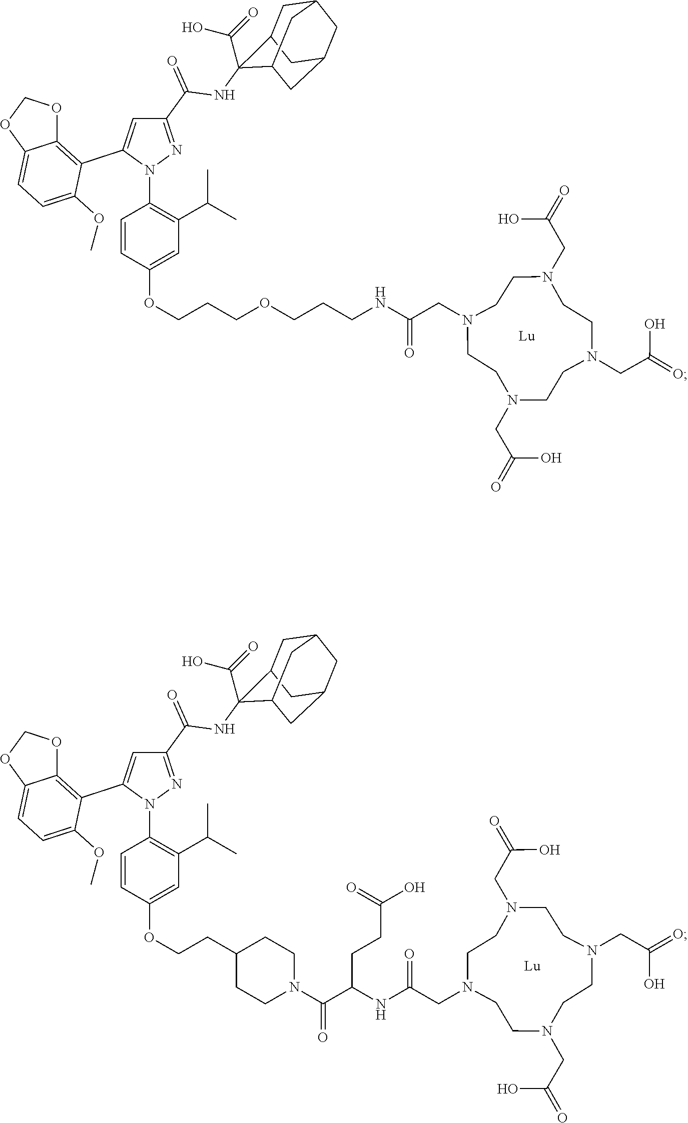

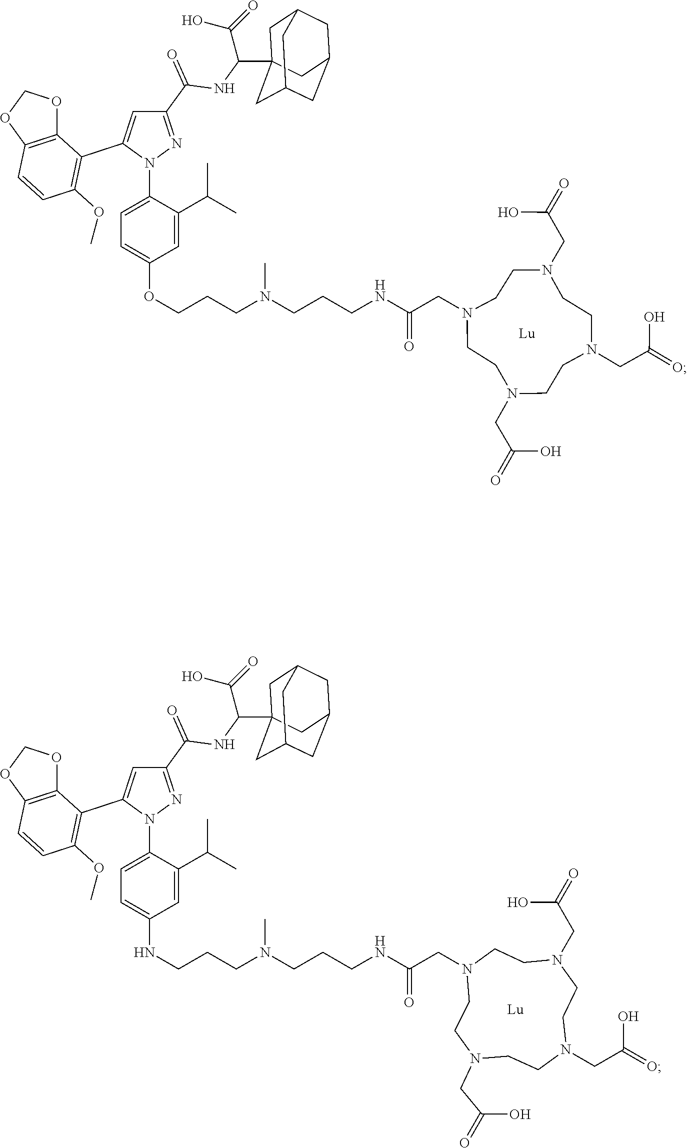

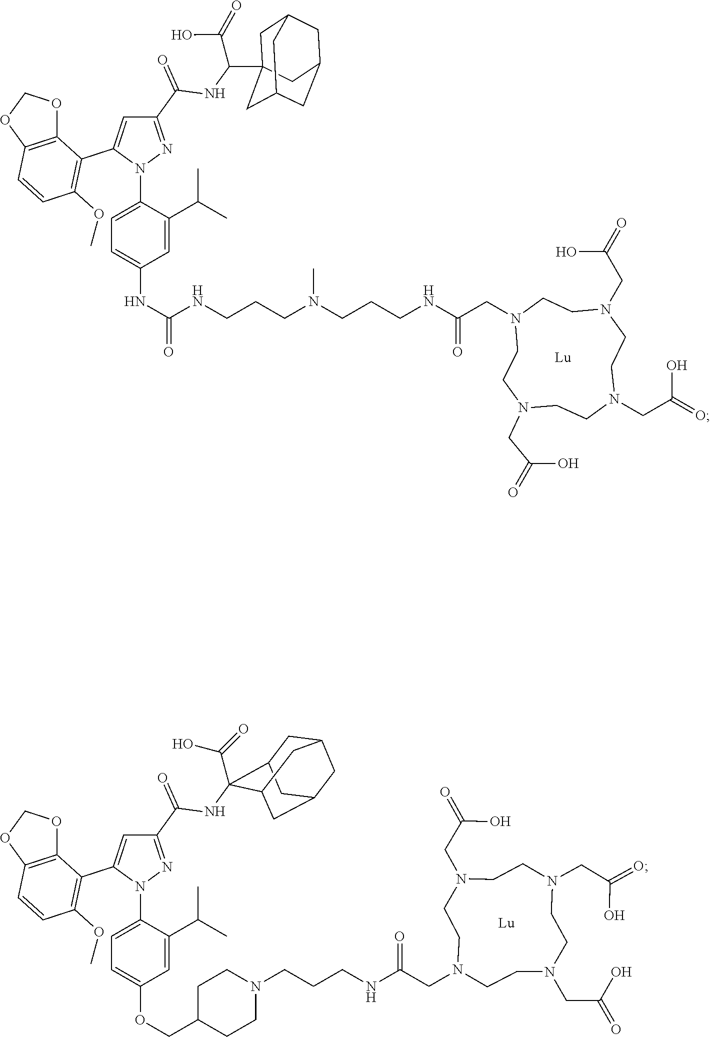

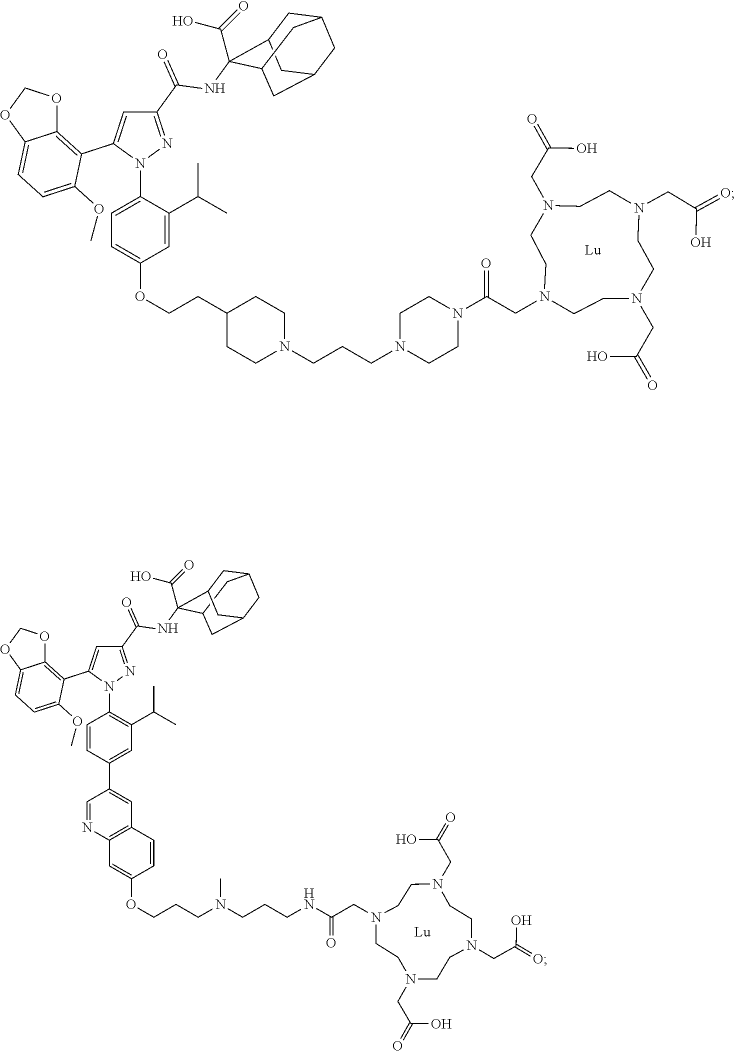

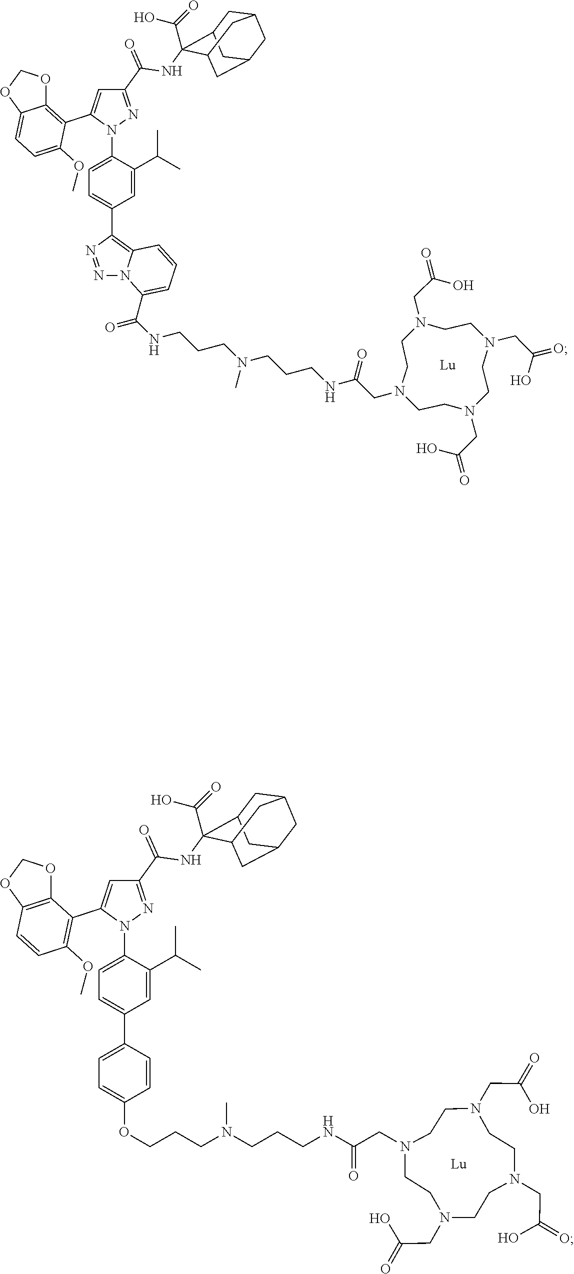

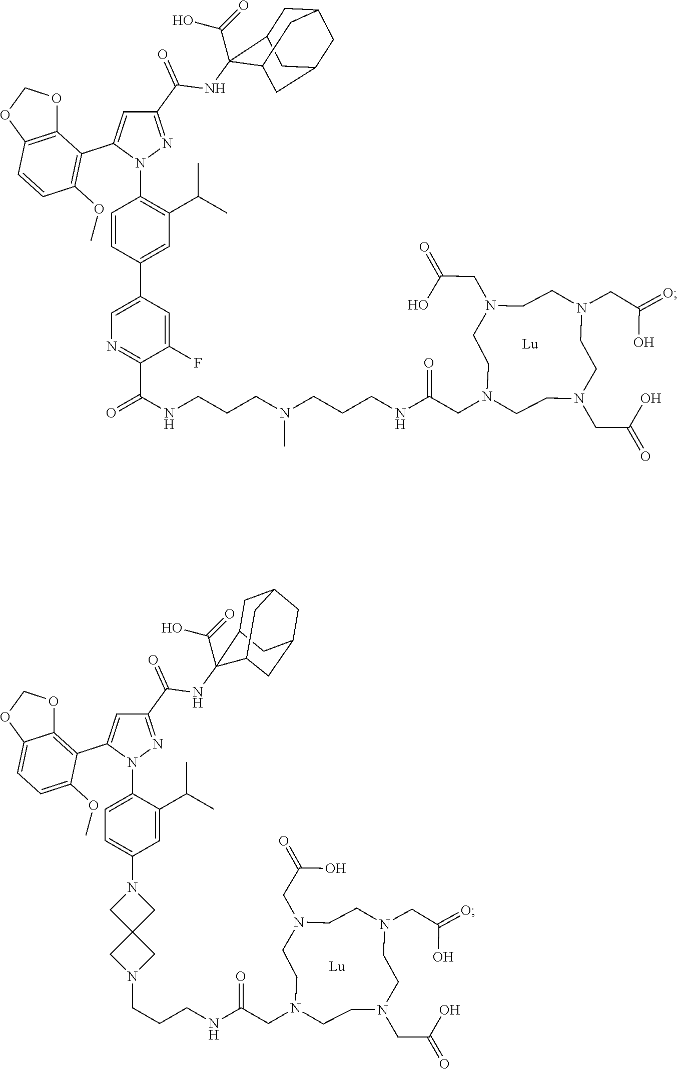

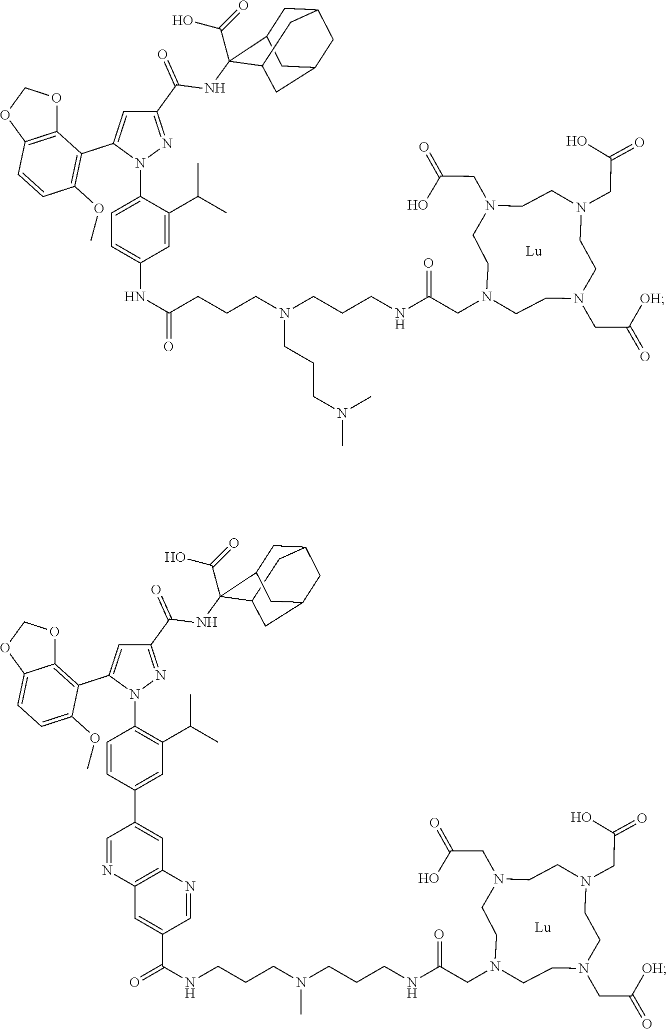

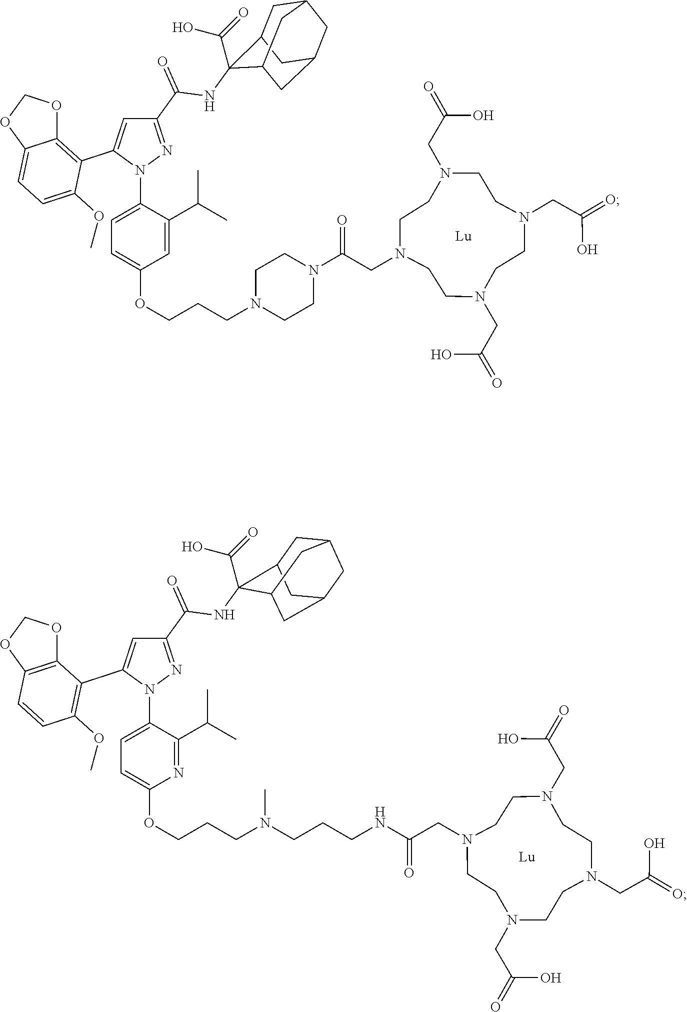

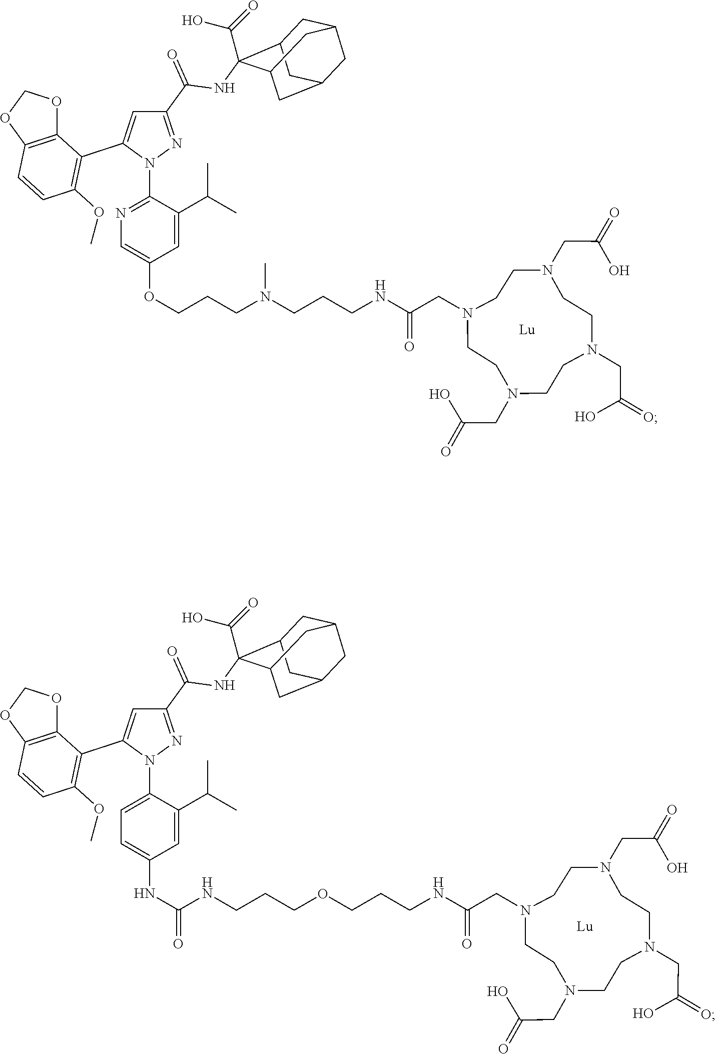

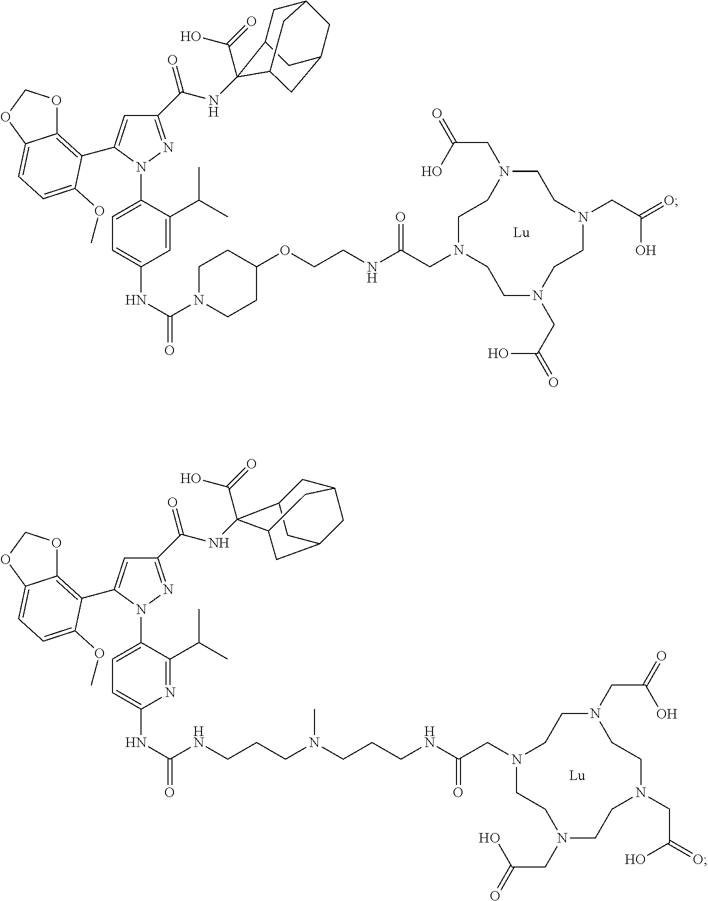

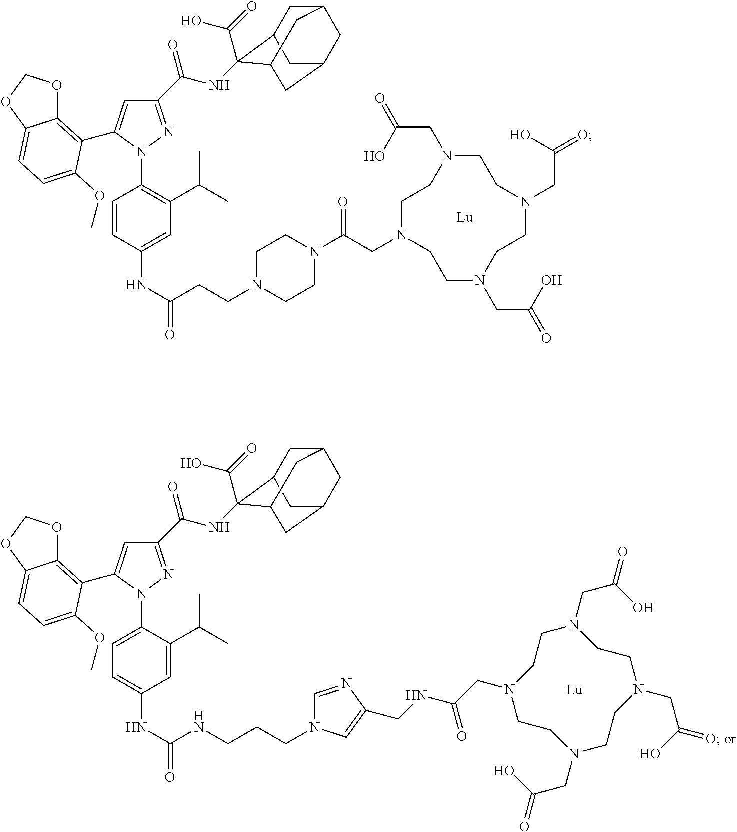

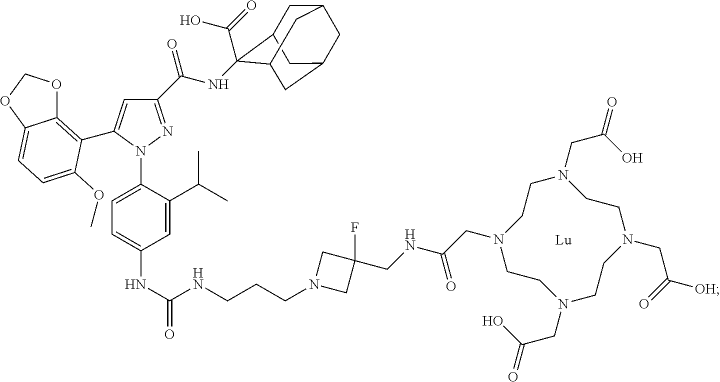

or a pharmaceutically acceptable salt thereof.

The present invention also provides a compound that is

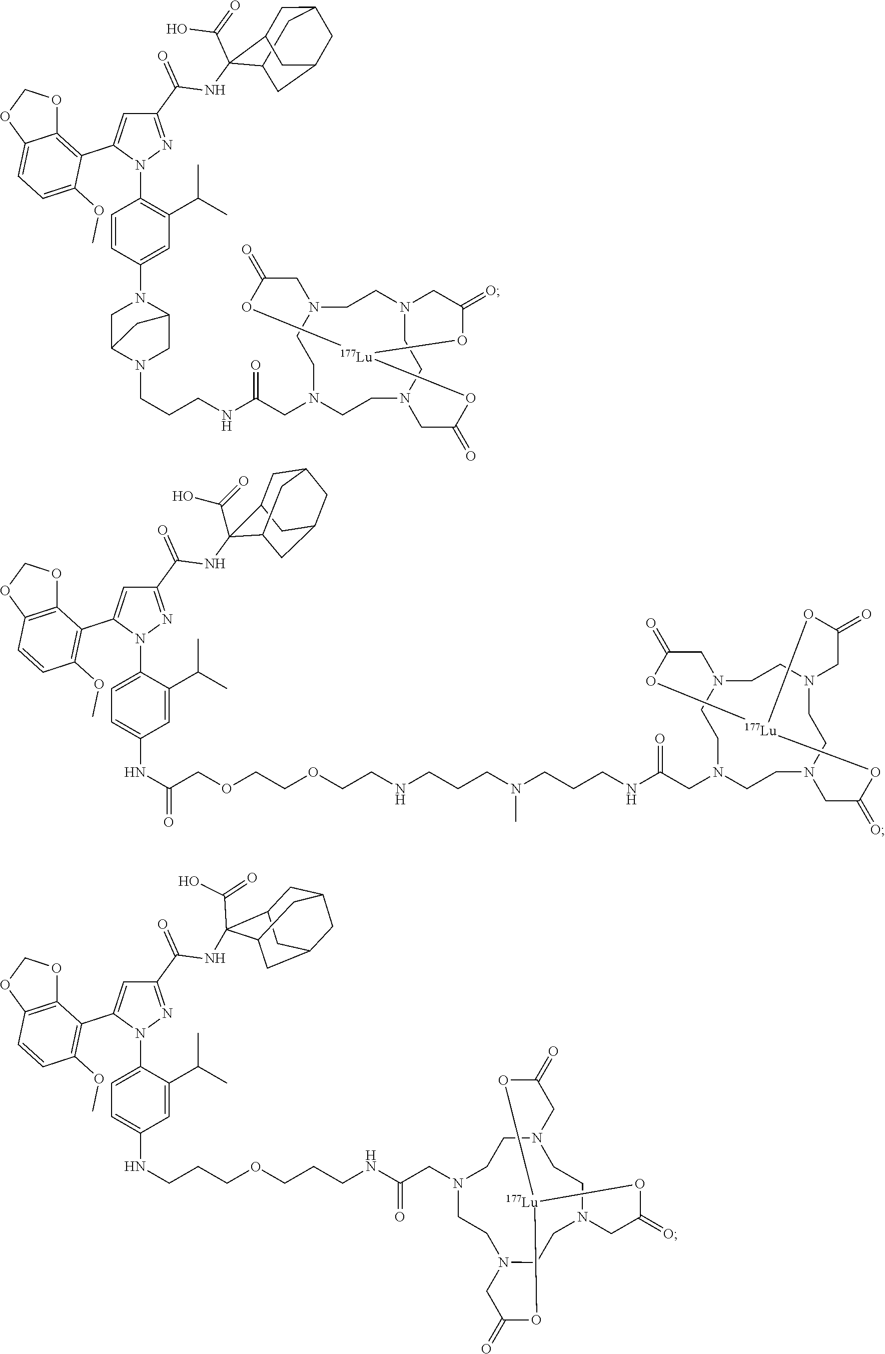

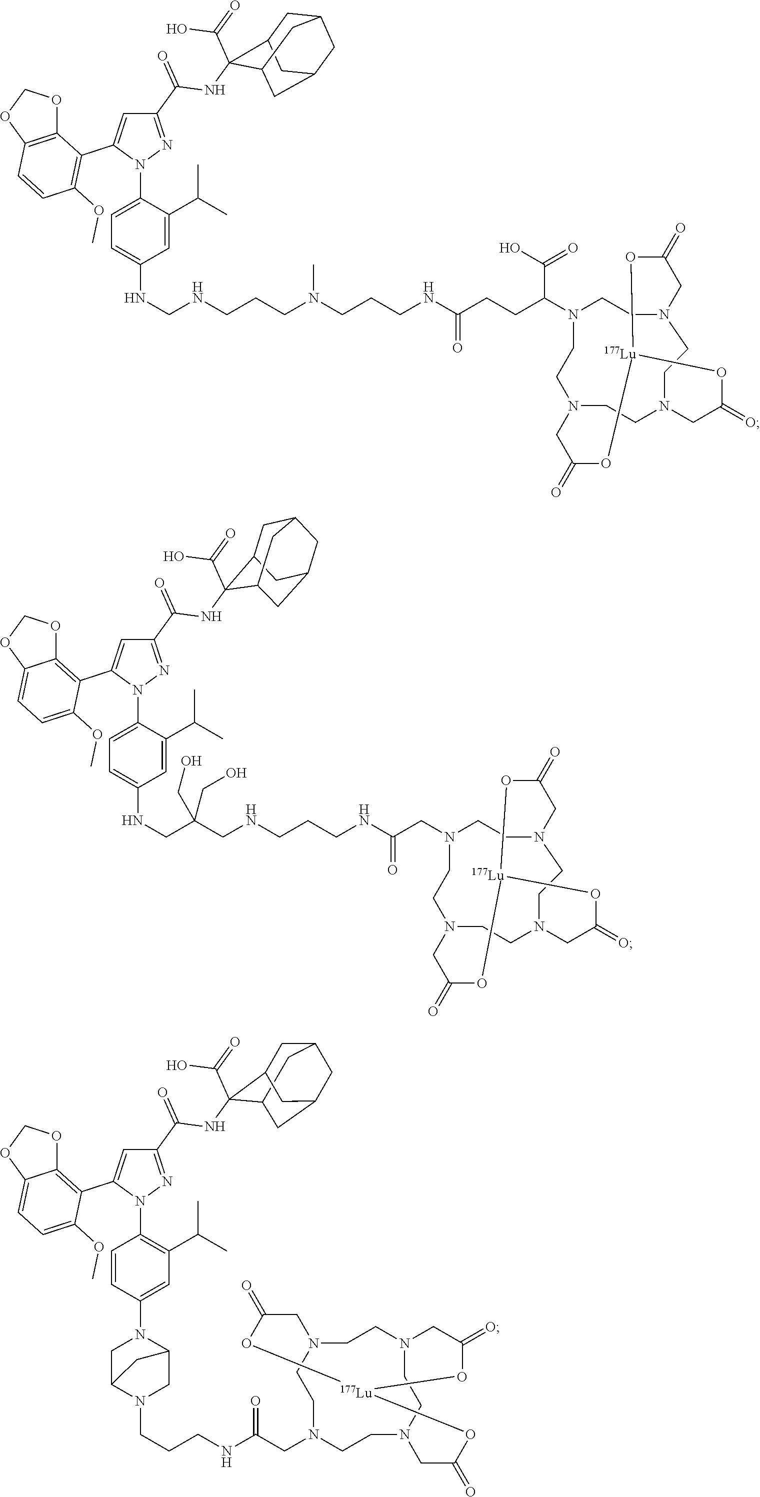

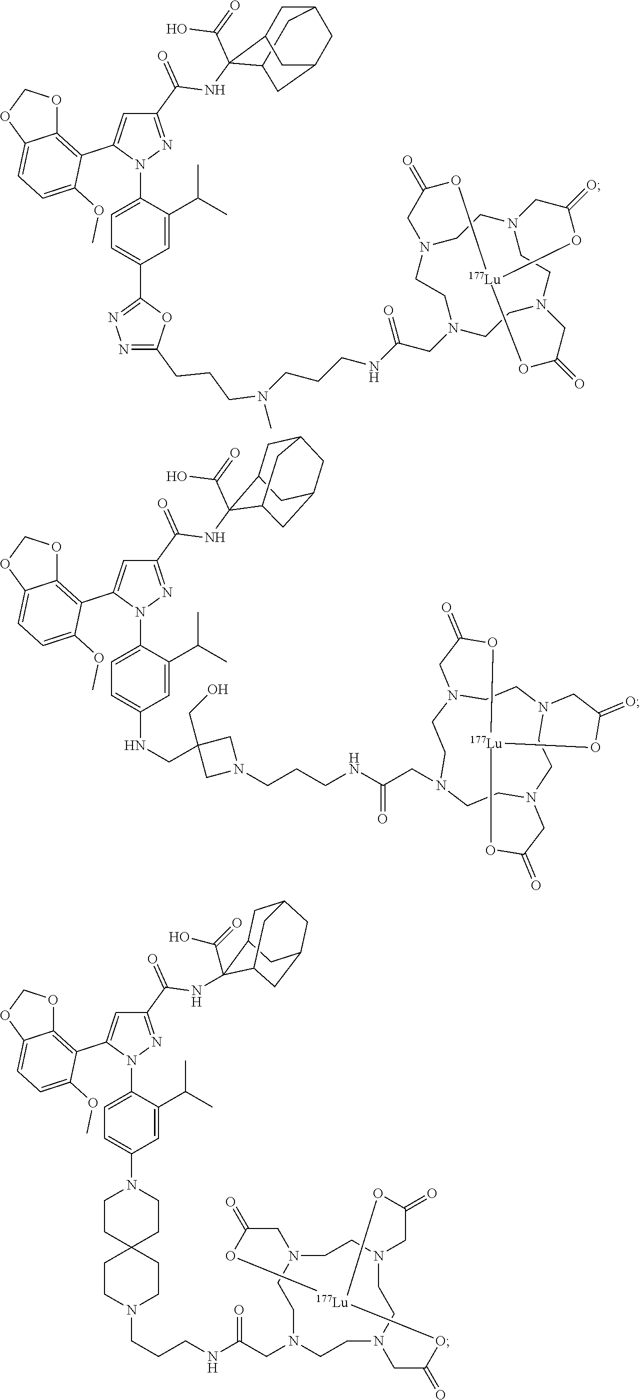

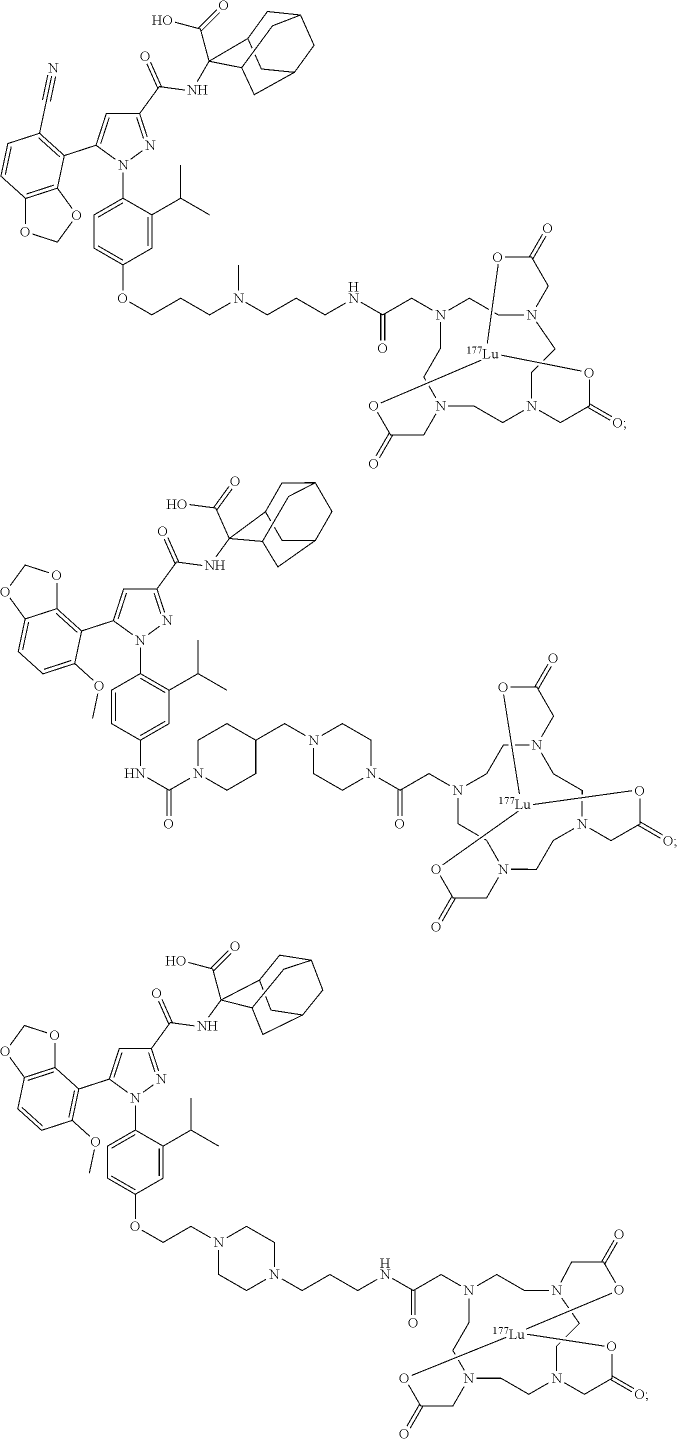

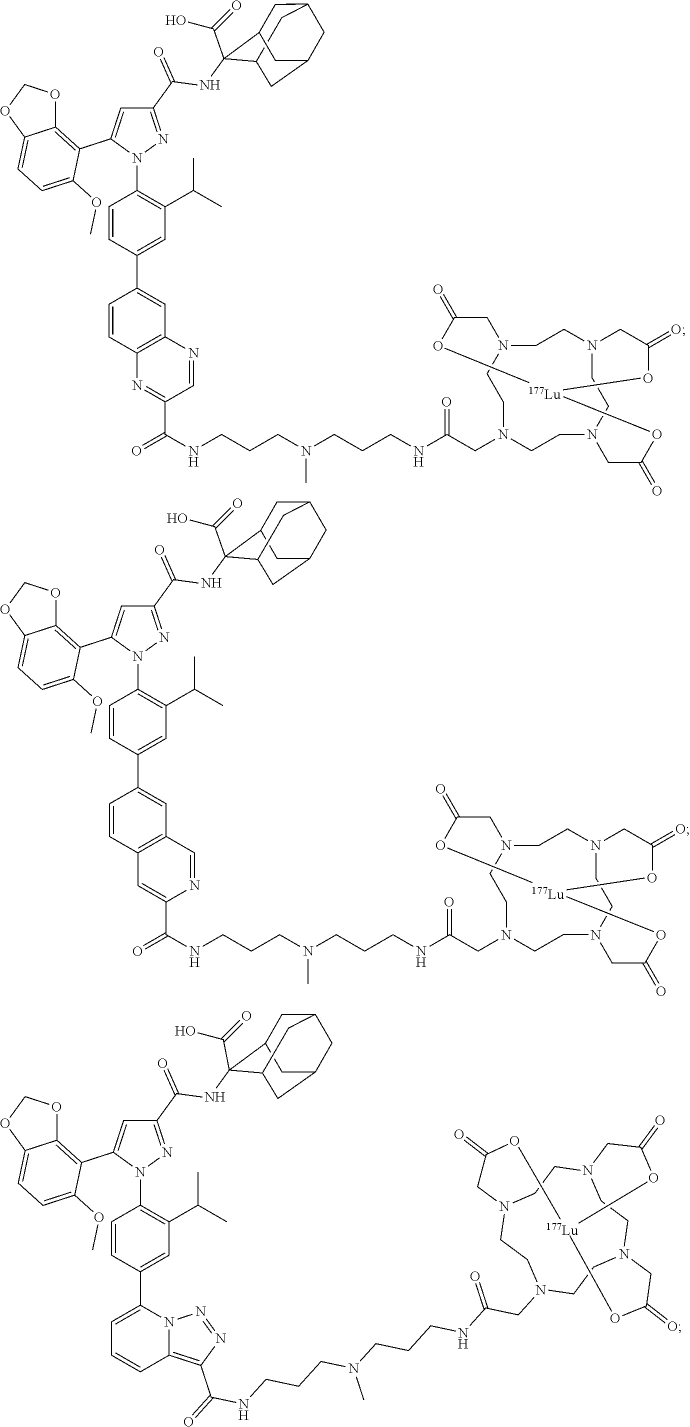

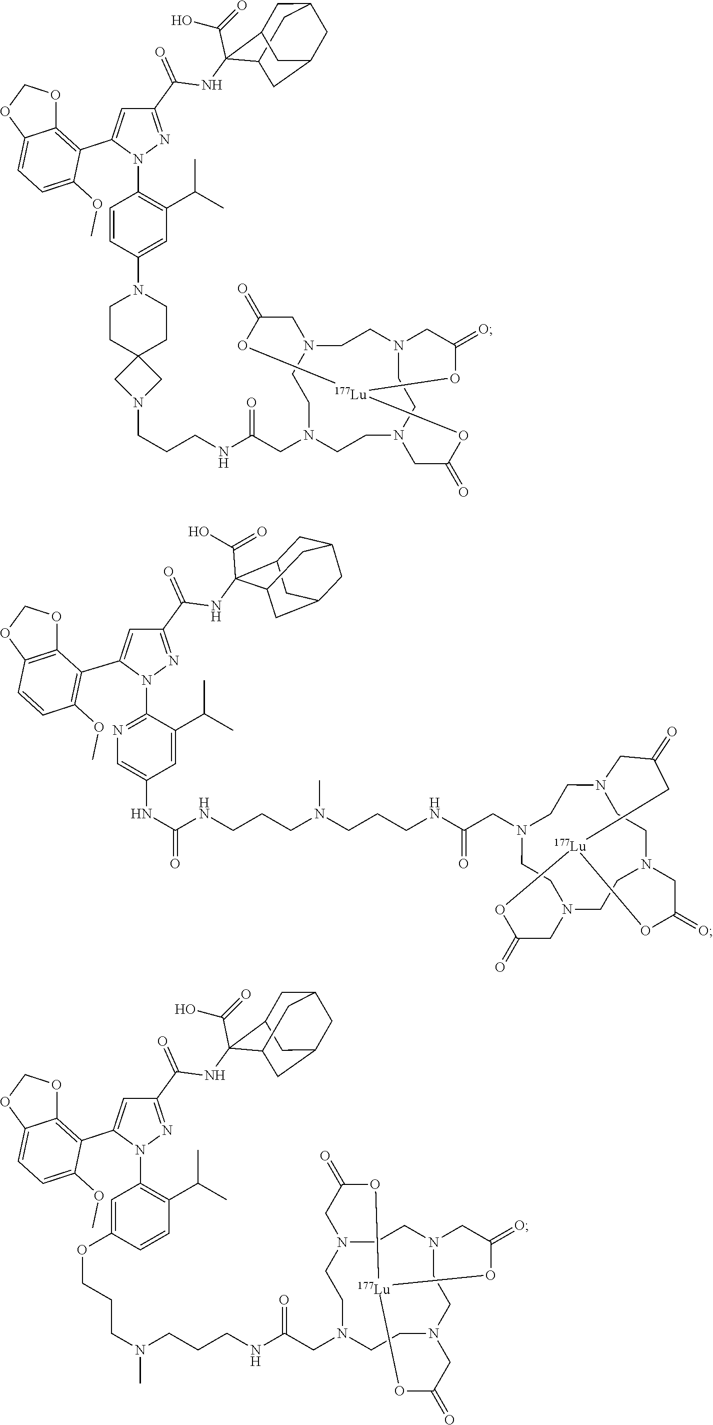

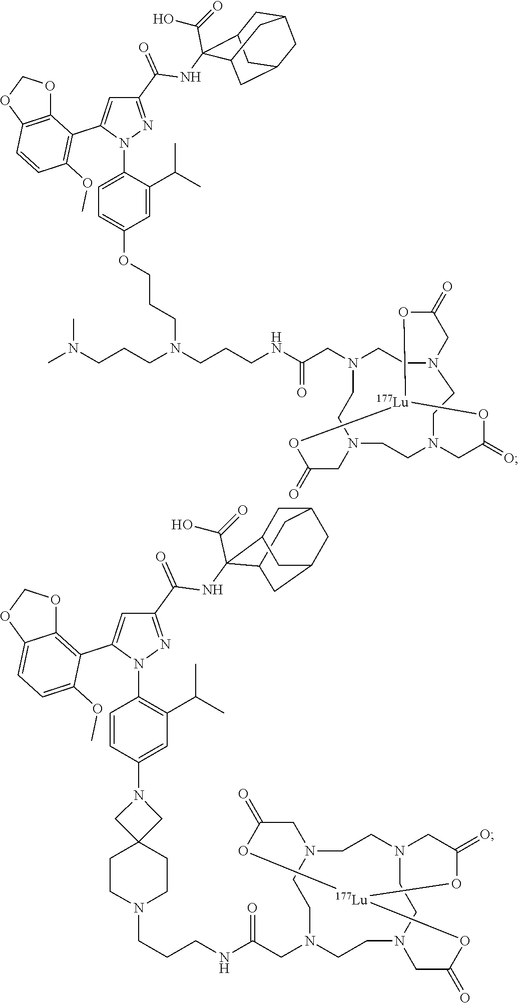

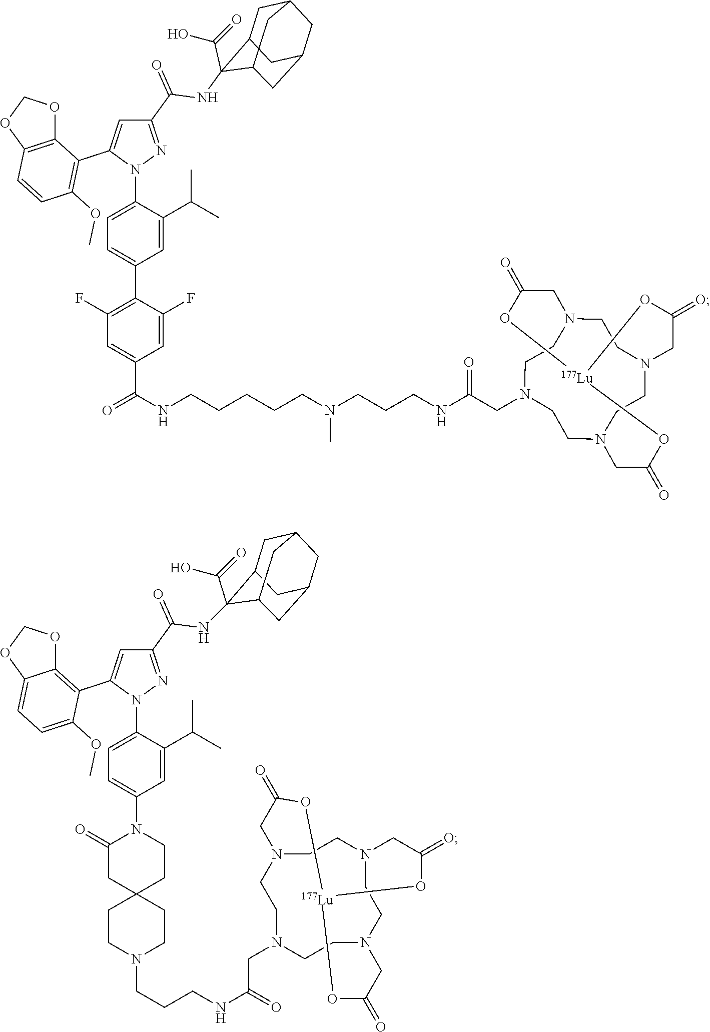

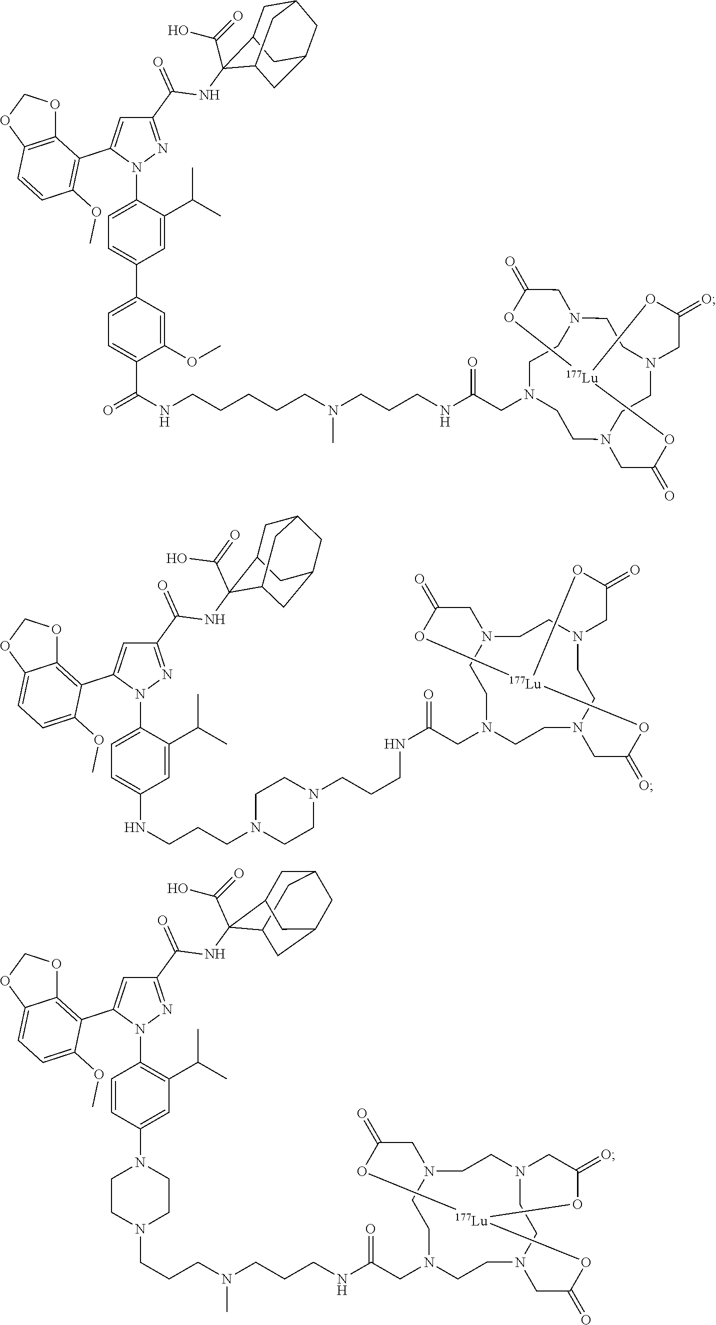

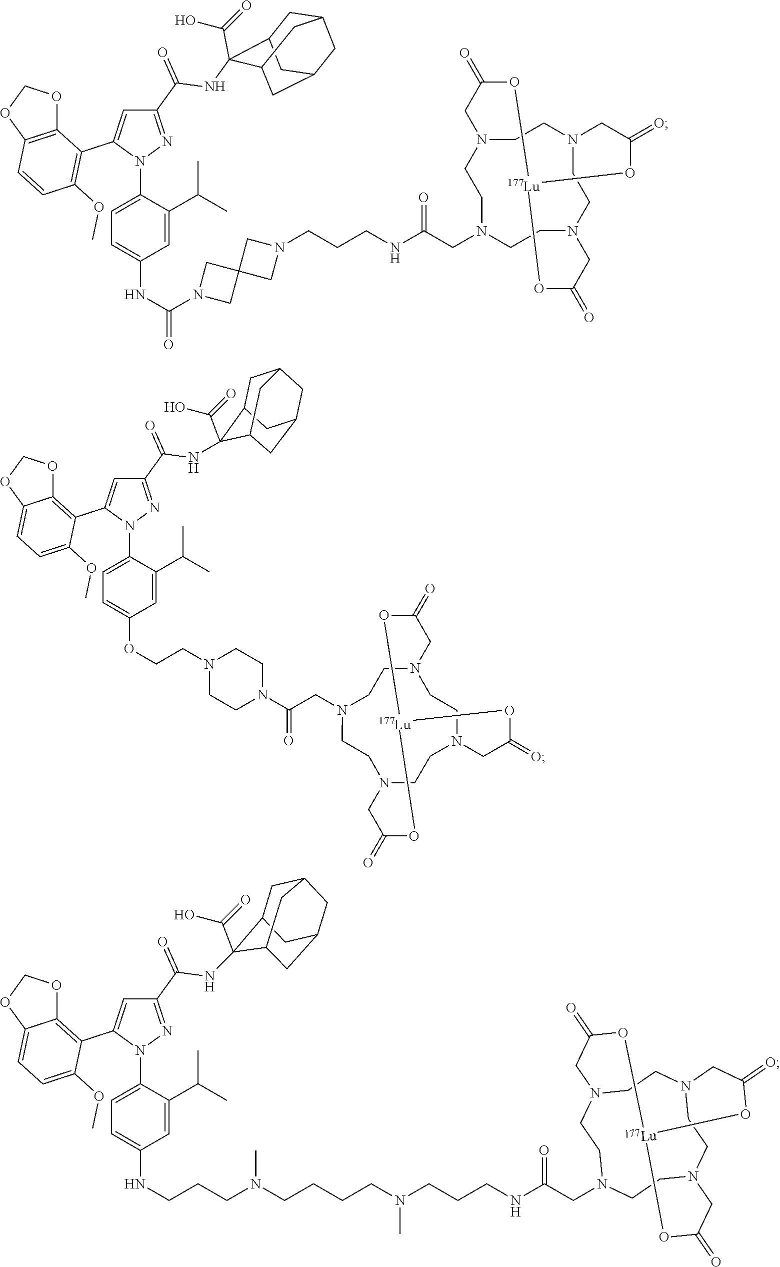

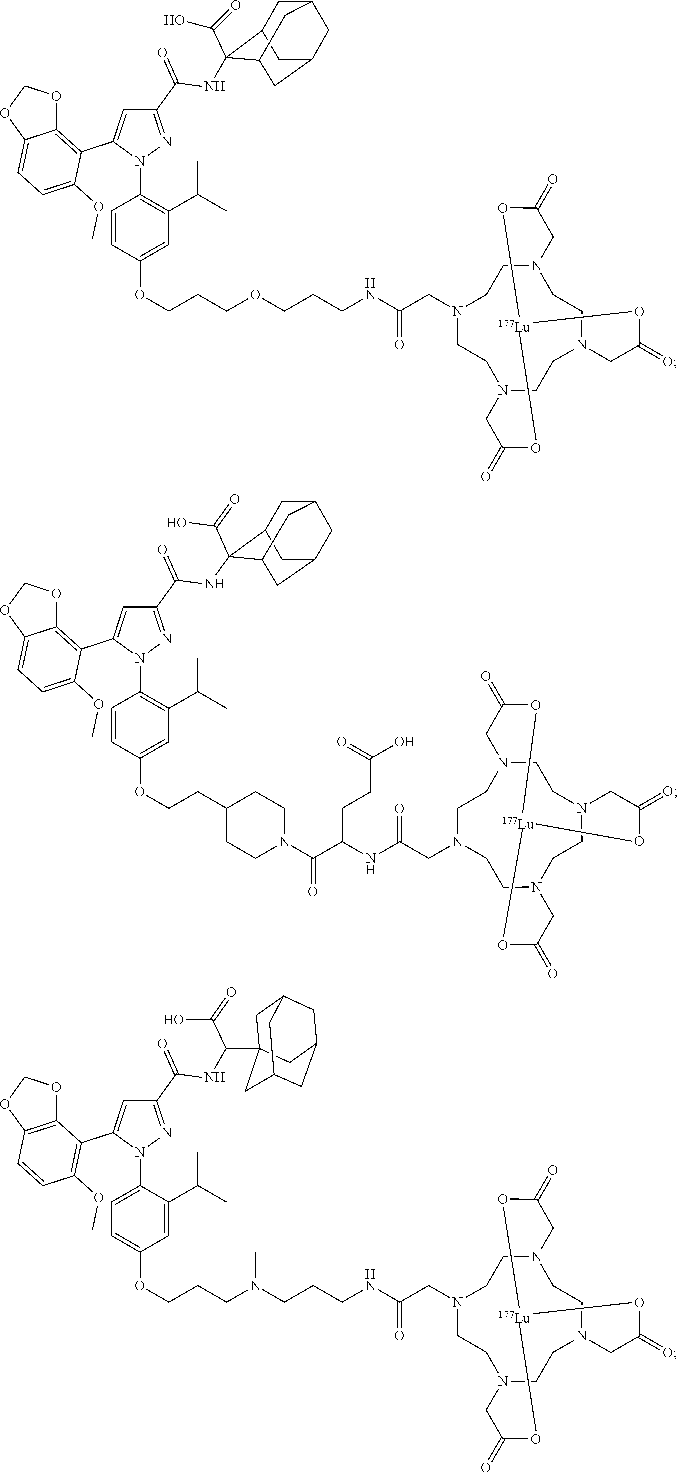

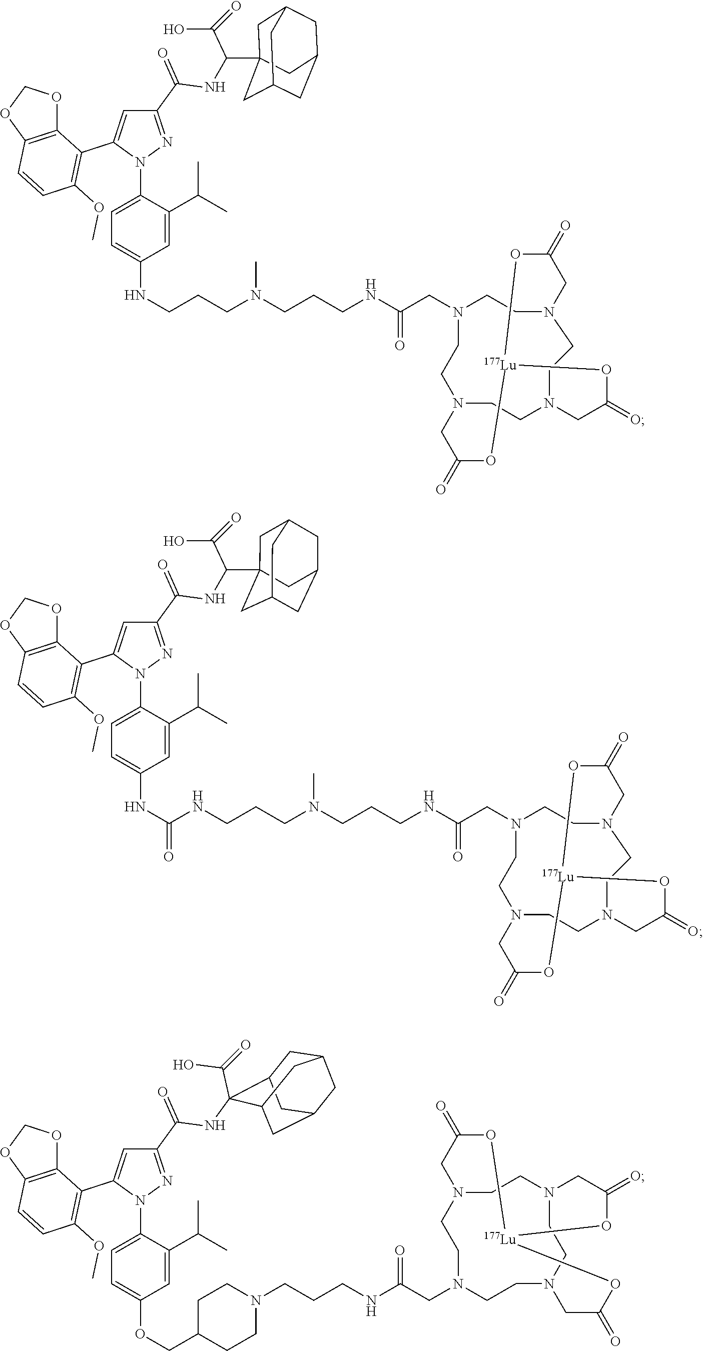

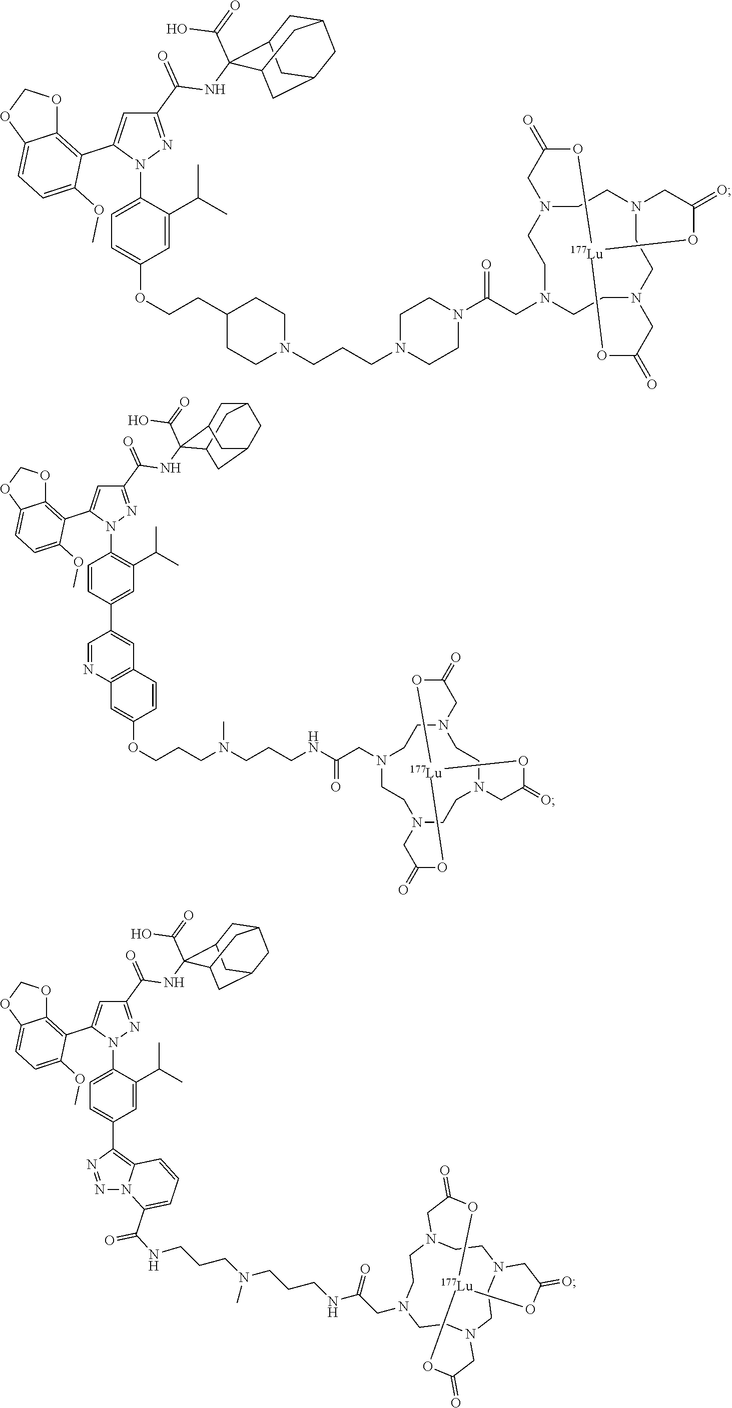

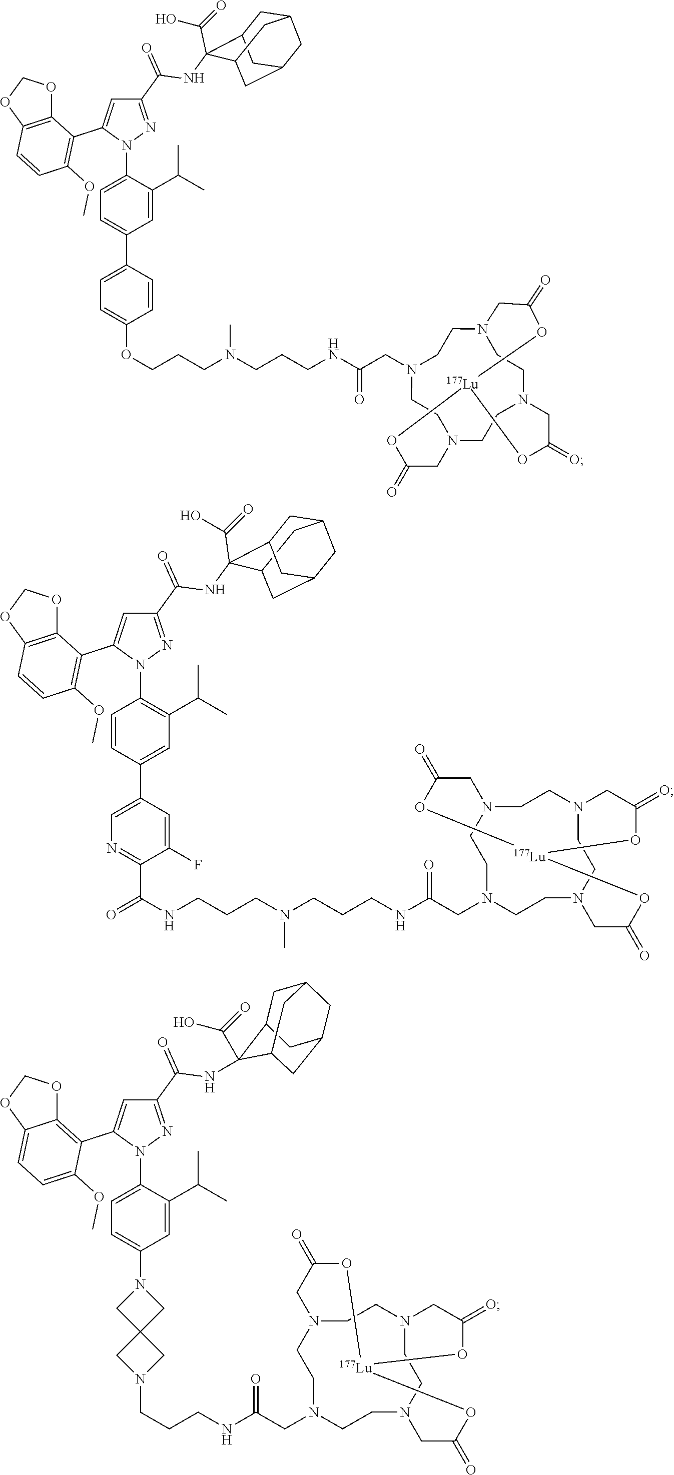

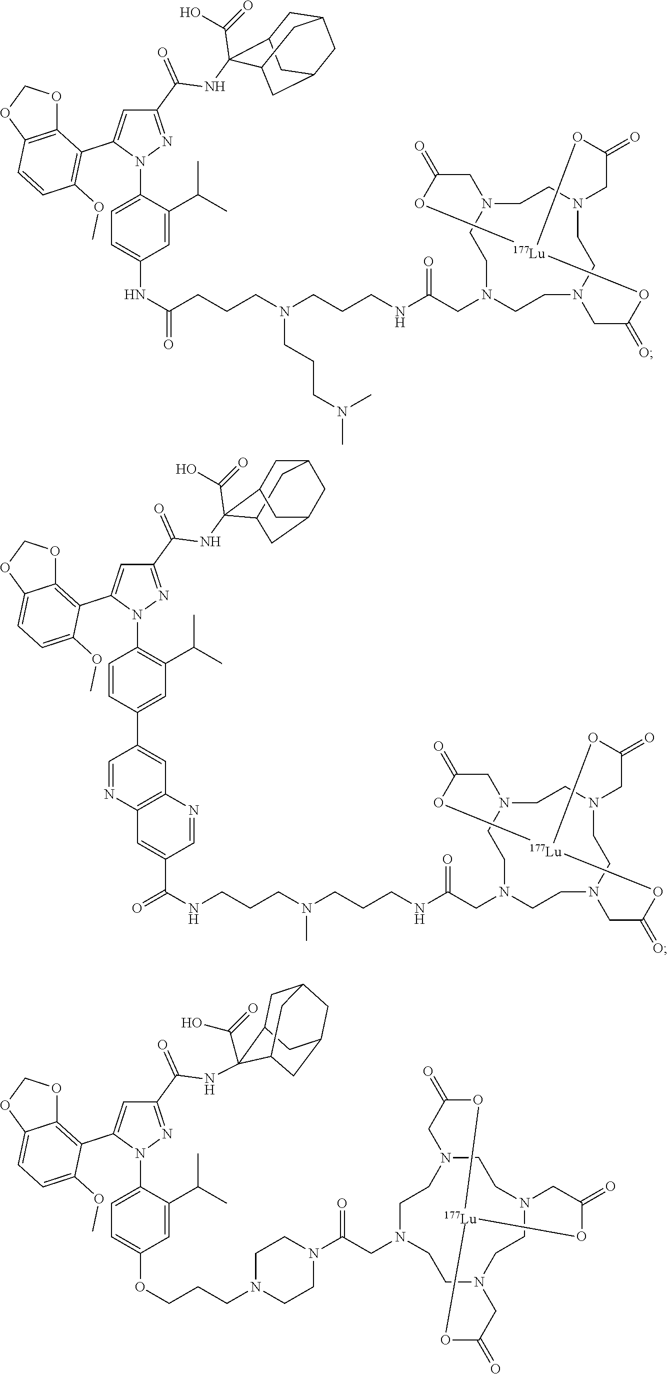

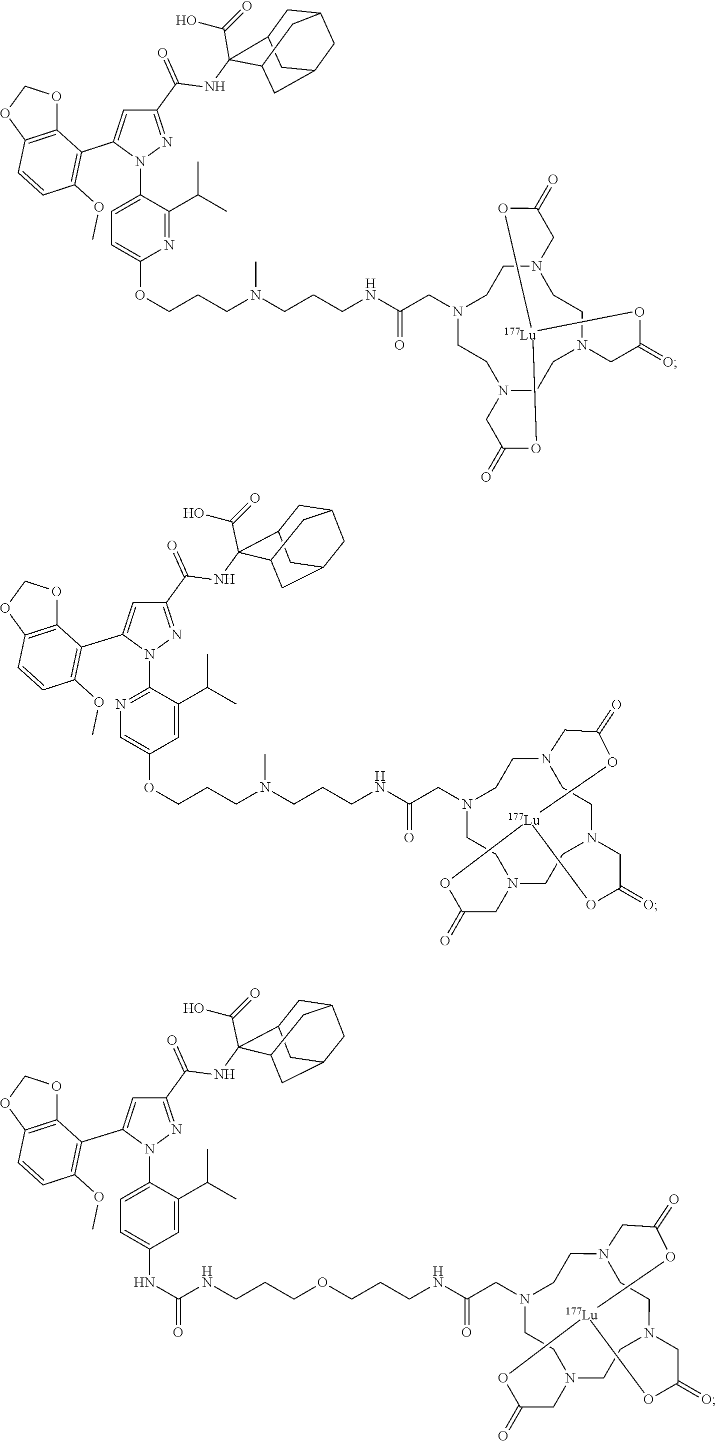

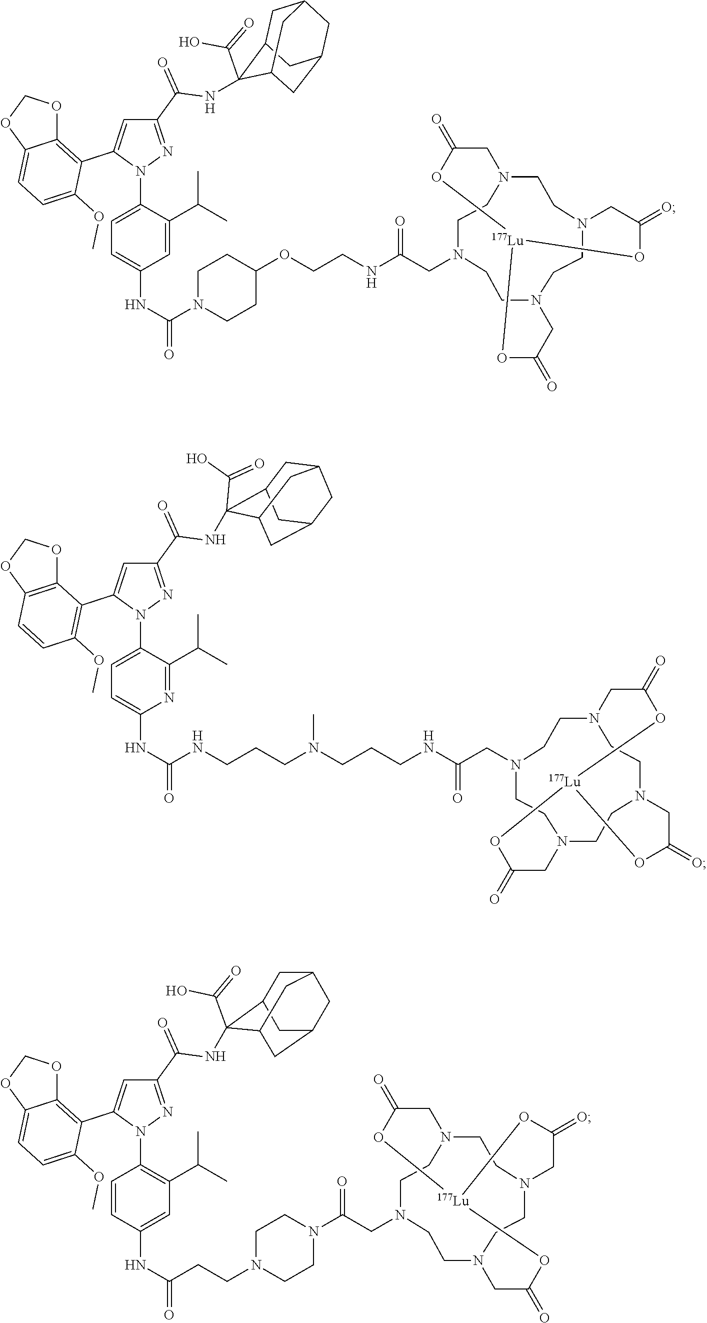

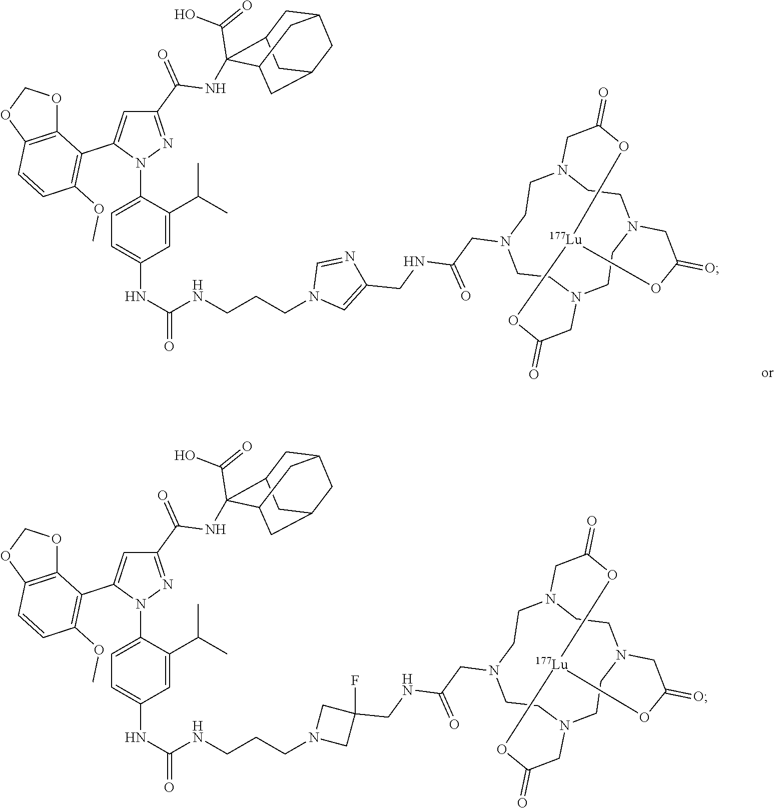

or a pharmaceutically acceptable salt thereof.

The present invention also provides a compound of Formula (II):

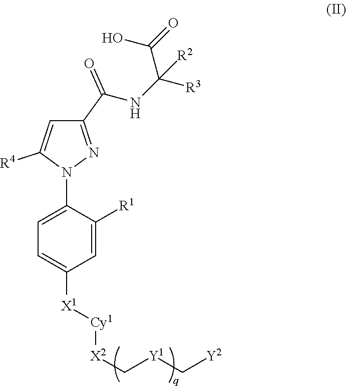

wherein R¹, R², R³, R⁴, X¹, Cy¹, and X² are as defined above; each Y¹ is independently a bond or —NR^{y1}—; Y² is —NR^{y2}R^{y3} or a 5- or 6-membered heterocycle selected from pyrrolidine, piperazine, or piperidine, where the 5- or 6-membered heterocycle is optionally substituted with a C₁-C₆ alkyl; R^{y1}, R^{y2}, and R^{y3} at each occurrence are independently selected from H and C₁-C₆ alkyl; and q is 0, 1, 2, 3, 4, 5, or 6; or a pharmaceutically acceptable salt thereof.

In an embodiment of a compound of Formula (II), or a pharmaceutically acceptable salt thereof, q is 3; Y¹ at each occurrence is a bond; and Y² is NR^{y2}R^{y3}.

In an embodiment of a compound of Formula (II), or a pharmaceutically acceptable salt thereof, q is 1; Y¹ is a bond; and Y² is a 5- or 6-membered heterocycle selected from pyrrolidine, piperazine, or piperidine, where the 5- or 6-membered heterocycle is optionally substituted with a C₁-C₆ alkyl.

In an embodiment of a compound of Formula (II), or a pharmaceutically acceptable salt thereof, q is 6; five occurrences of Y¹ are a bond, and one occurrence of Y¹ is —NR^{y1}—, and Y² is NR^{y2}R^{y3}.

In an embodiment of a compound of Formula (II), or a pharmaceutically acceptable salt thereof, —X²—(CH₂-Y¹)_{q}-CH₂-Y² is selected from:

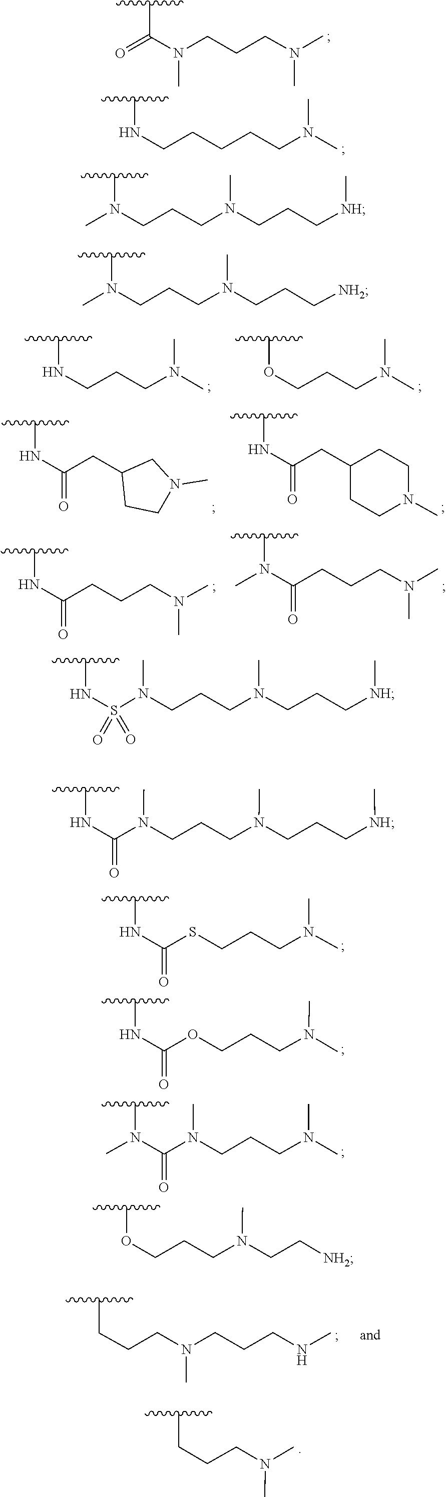

The present invention also provides a compound of Formula (II-a):

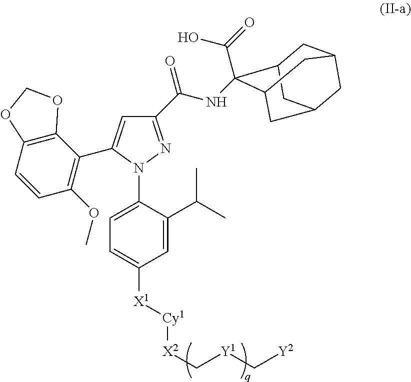

wherein X¹, Cy¹, X², Y¹, Y², and q are as defined above; or a pharmaceutically acceptable salt thereof.

The present invention also provides a compound selected from:

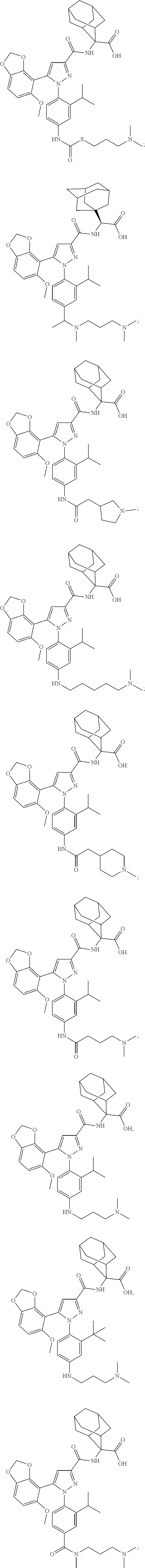

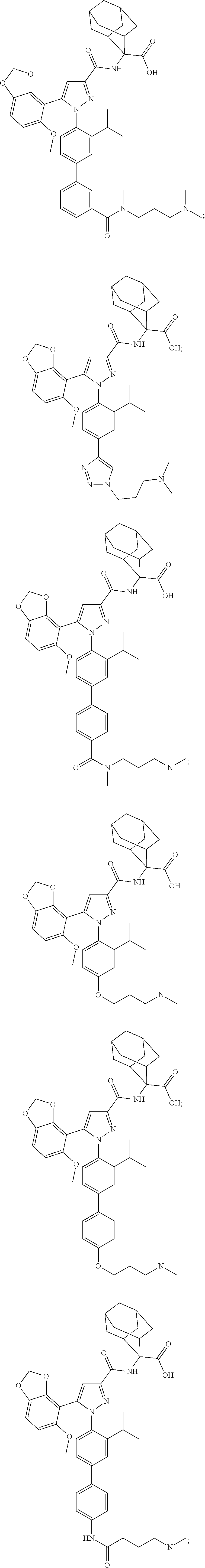

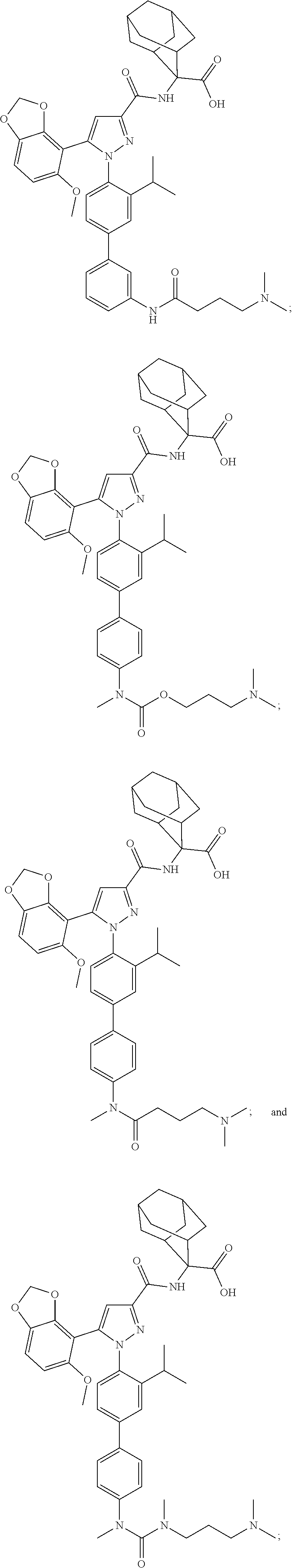 [SMILES: COc1ccc2c(c1-c1cc(C(=O)NC3(C(=O)O)C4CC5CC(C4)CC3C5)nn1-c1ccc(-c3cccc(NC(=O)CCCN(C)C)c3)cc1C(C)C)OCO2 ; COc1ccc2c(c1-c1cc(C(=O)NC3(C(=O)O)C4CC5CC(C4)CC3C5)nn1-c1ccc(-c3ccc(N(C)C(=O)OCCCN(C)C)cc3)cc1C(C)C)OCO2 ; COc1ccc2c(c1-c1cc(C(=O)NC3(C(=O)O)C4CC5CC(C4)CC3C5)nn1-c1ccc(-c3ccc(N(C)C(=O)CCCN(C)C)cc3)cc1C(C)C)OCO2 ; COc1ccc2c(c1-c1cc(C(=O)NC3(C(=O)O)C4CC5CC(C4)CC3C5)nn1-c1ccc(-c3ccc(N(C)C(=O)N(C)CCCN(C)C)cc3)cc1C(C)C)OCO2]

or a pharmaceutically acceptable salt thereof.

The present invention also provides a compound of Formula (III):

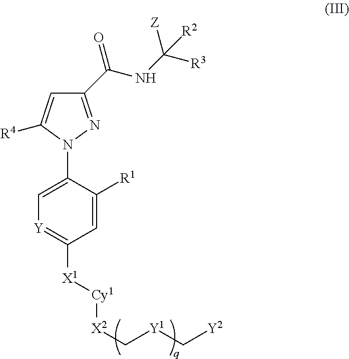

wherein R¹, R², R³, Z, R⁴, Y, X¹, Cy¹, and X² are as defined above; each Y¹ is independently a bond or —NR^{y1}—; Y² is —NR^{y2}R^{y3} or a 5- or 6-membered heterocycle that is pyrrolidine, piperazine, or piperidine, where the 5- or 6-membered heterocycle is optionally substituted with a C₁-C₆ alkyl; R^{y1}, R^{y2}, and R^{y3} at each occurrence are independently selected from H and C₁-C₆ alkyl; and q is 0, 1, 2, 3, 4, 5, or 6; or a pharmaceutically acceptable salt thereof.

In an embodiment of a compound of Formula (II), or a pharmaceutically acceptable salt thereof, q is 3; Y¹ at each occurrence is a bond; and Y² is NR^{y2}R^{y3}.

In an embodiment of a compound of Formula (II), or a pharmaceutically acceptable salt thereof, q is 1; Y¹ is a bond; and Y² is a 5- or 6-membered heterocycle that is pyrrolidine, piperazine, or piperidine, where the 5- or 6-membered heterocycle is optionally substituted with a C₁-C₆ alkyl.

In an embodiment of a compound of Formula (II), or a pharmaceutically acceptable salt thereof, q is 6; five occurrences of Y¹ are a bond, and one occurrence of Y¹ is —NR^{y1}—, and Y² is NR^{y2}R^{y3}.

In an embodiment of a compound of Formula (II), or a pharmaceutically acceptable salt thereof, —X²—(CH₂-Y¹)_{q}-CH₂-Y² is

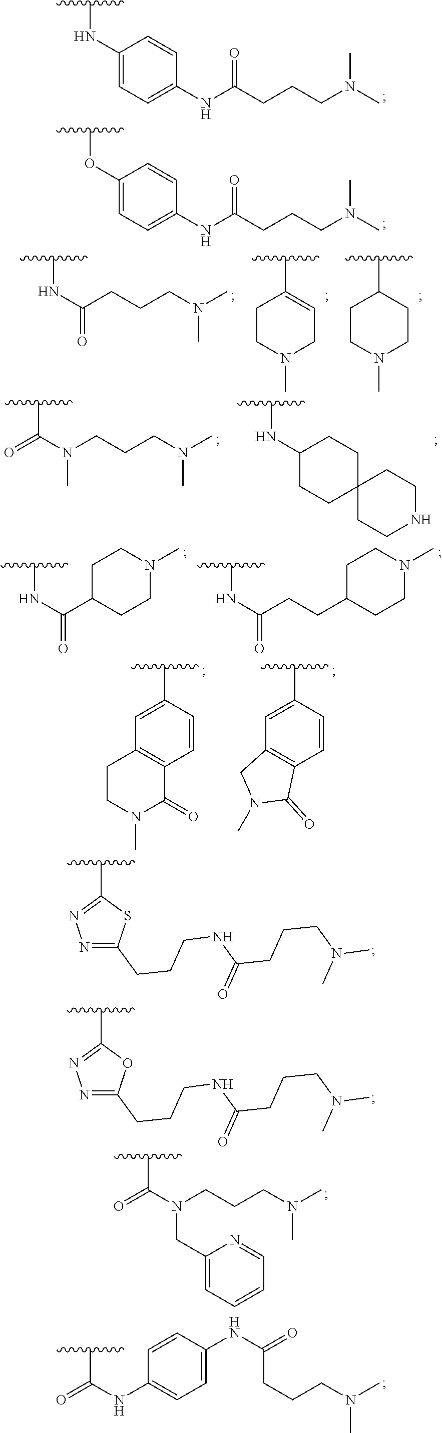

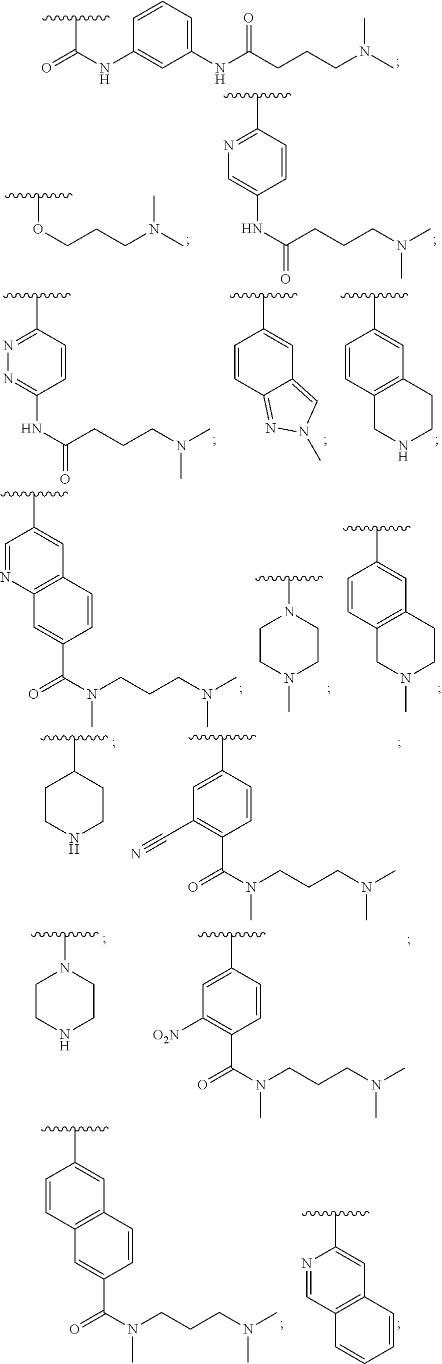

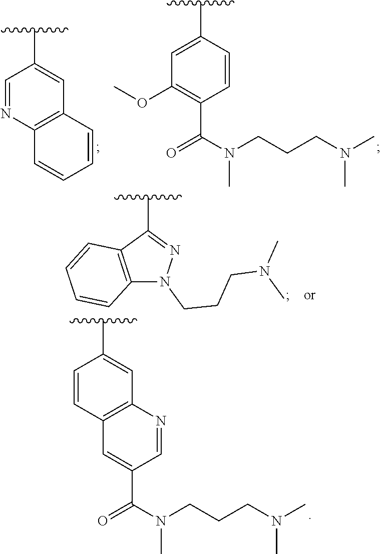

The present invention also provides a compound of Formula (III-a):

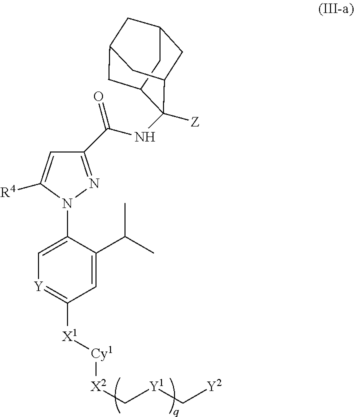

wherein Z, R⁴, Y, X¹, Cy¹, X², Y¹, Y², and q are as defined above; or a pharmaceutically acceptable salt thereof.

The present invention also provides a compound that is

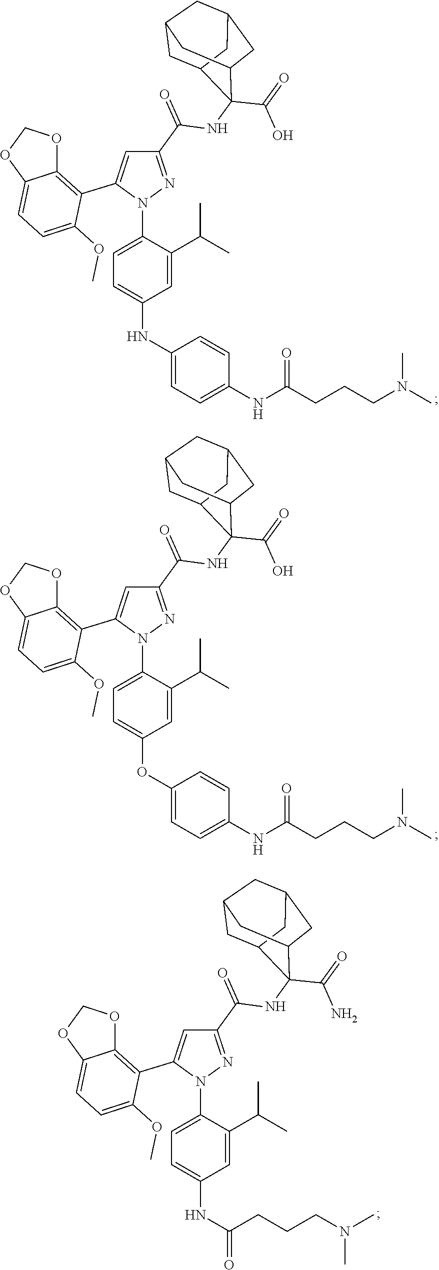

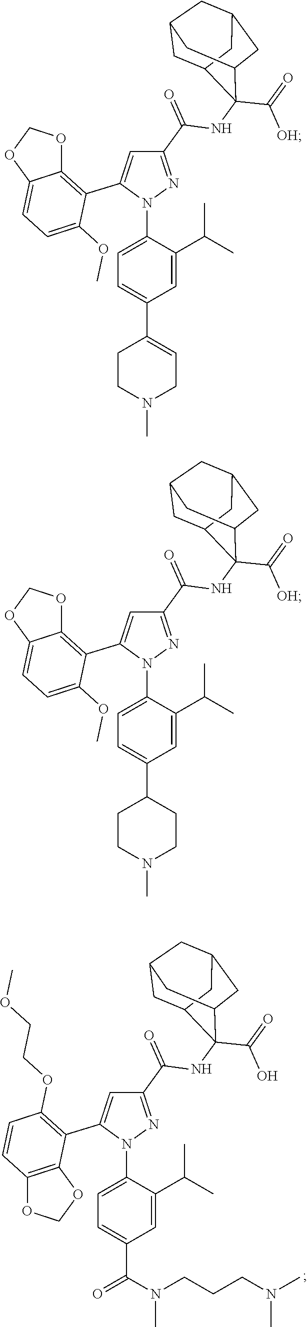 [SMILES: COc1ccc2c(c1-c1cc(C(=O)NC3(C(=O)O)C4CC5CC(C4)CC3C5)nn1-c1ccc(C3=CCN(C)CC3)cc1C(C)C)OCO2 ; COc1ccc2c(c1-c1cc(C(=O)NC3(C(=O)O)C4CC5CC(C4)CC3C5)nn1-c1ccc(C3CCN(C)CC3)cc1C(C)C)OCO2 ; COCCOc1ccc2c(c1-c1cc(C(=O)NC3(C(=O)O)C4CC5CC(C4)CC3C5)nn1-c1ccc(C(=O)N(C)CCCN(C)C)cc1C(C)C)OCO2]

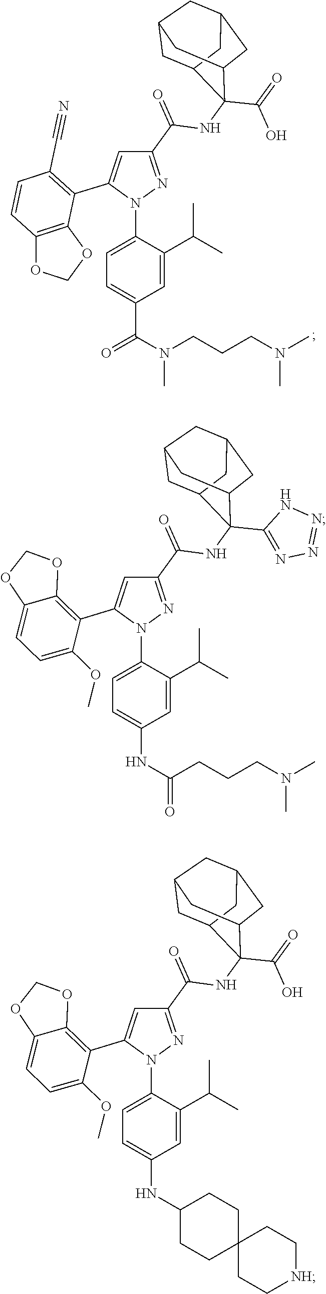 [SMILES: CC(C)c1cc(C(=O)N(C)CCCN(C)C)ccc1-n1nc(C(=O)NC2(C(=O)O)C3CC4CC(C3)CC2C4)cc1-c1c(C#N)ccc2c1OCO2 ; COc1ccc2c(c1-c1cc(C(=O)NC3(c4nnn[nH]4)C4CC5CC(C4)CC3C5)nn1-c1ccc(NC(=O)CCCN(C)C)cc1C(C)C)OCO2 ; COc1ccc2c(c1-c1cc(C(=O)NC3(C(=O)O)C4CC5CC(C4)CC3C5)nn1-c1ccc(NC3CCC4(CCNCC4)CC3)cc1C(C)C)OCO2]

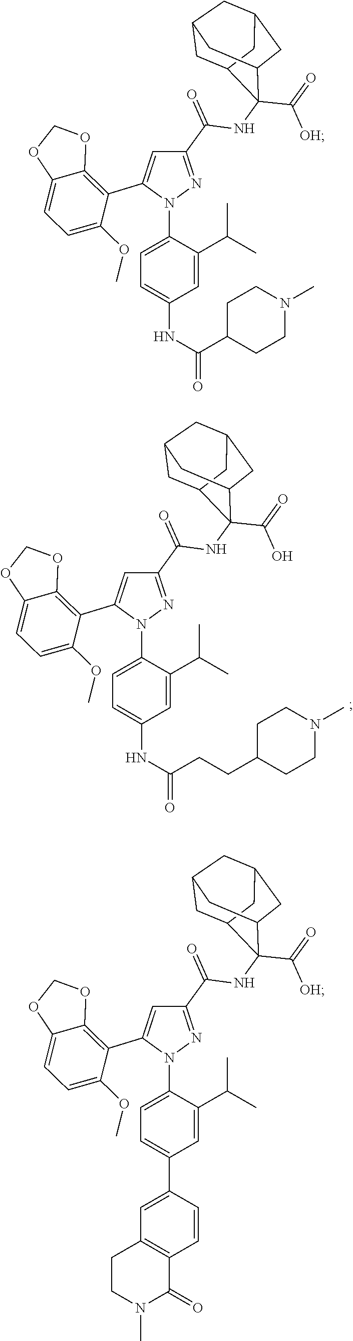

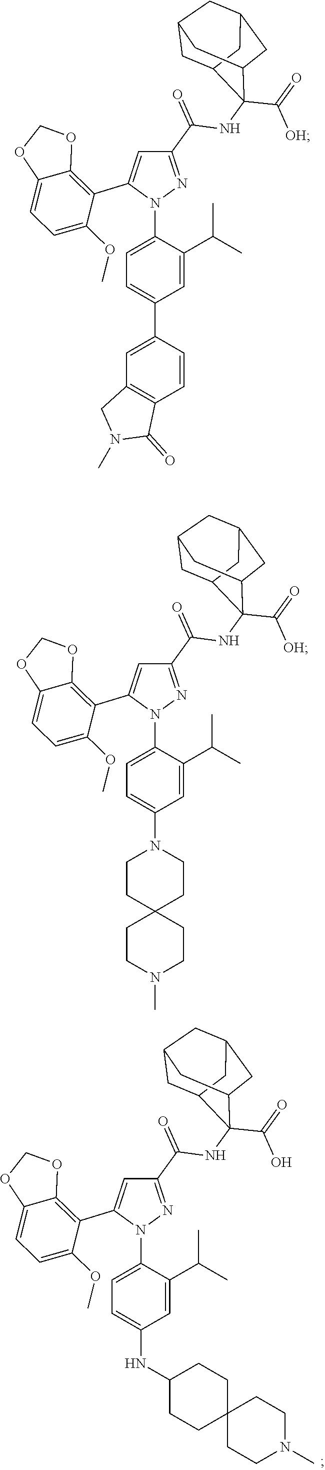

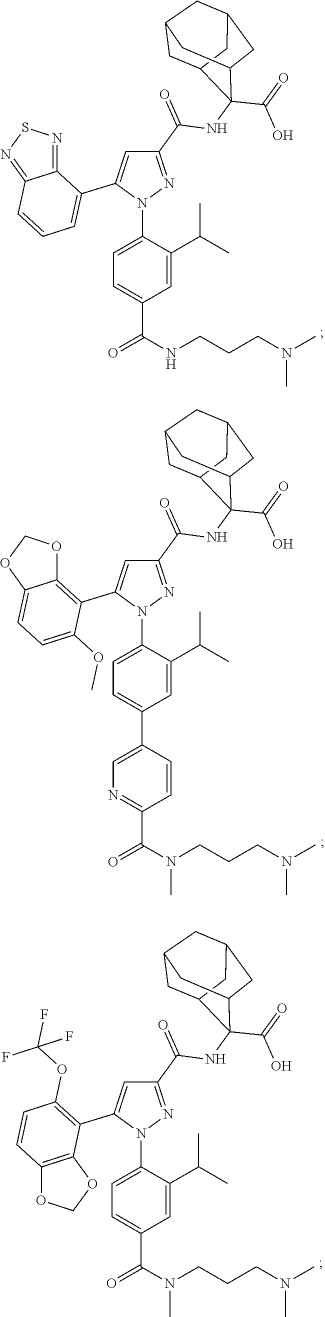

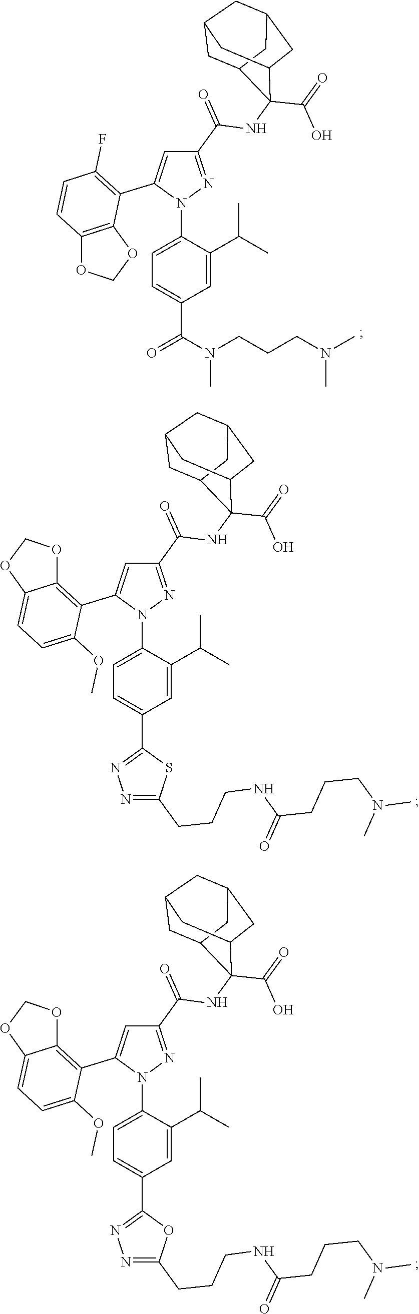

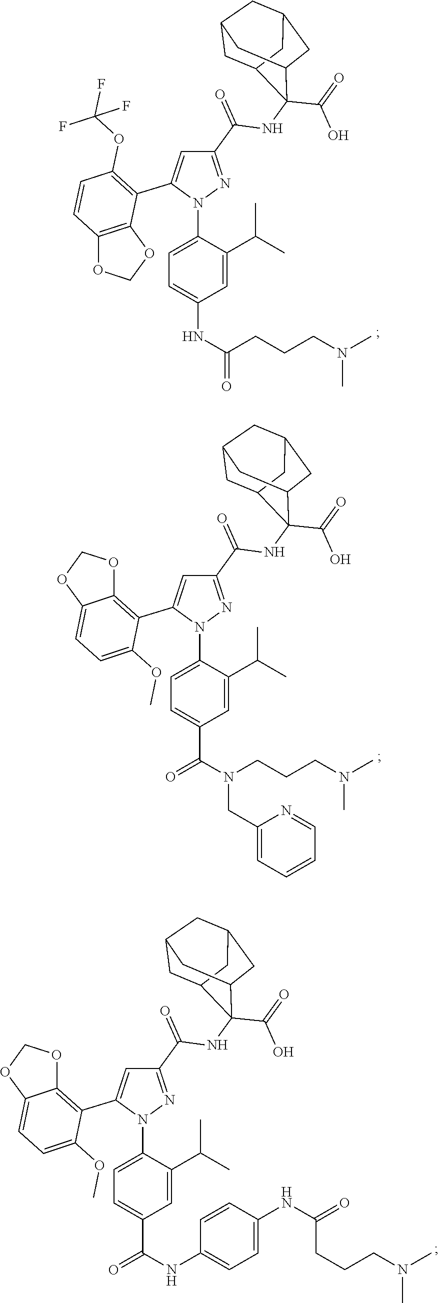 [SMILES: CC(C)c1cc(NC(=O)CCCN(C)C)ccc1-n1nc(C(=O)NC2(C(=O)O)C3CC4CC(C3)CC2C4)cc1-c1c(OC(F)(F)F)ccc2c1OCO2 ; COc1ccc2c(c1-c1cc(C(=O)NC3(C(=O)O)C4CC5CC(C4)CC3C5)nn1-c1ccc(C(=O)N(CCCN(C)C)Cc3ccccn3)cc1C(C)C)OCO2 ; COc1ccc2c(c1-c1cc(C(=O)NC3(C(=O)O)C4CC5CC(C4)CC3C5)nn1-c1ccc(C(=O)Nc3ccc(NC(=O)CCCN(C)C)cc3)cc1C(C)C)OCO2]

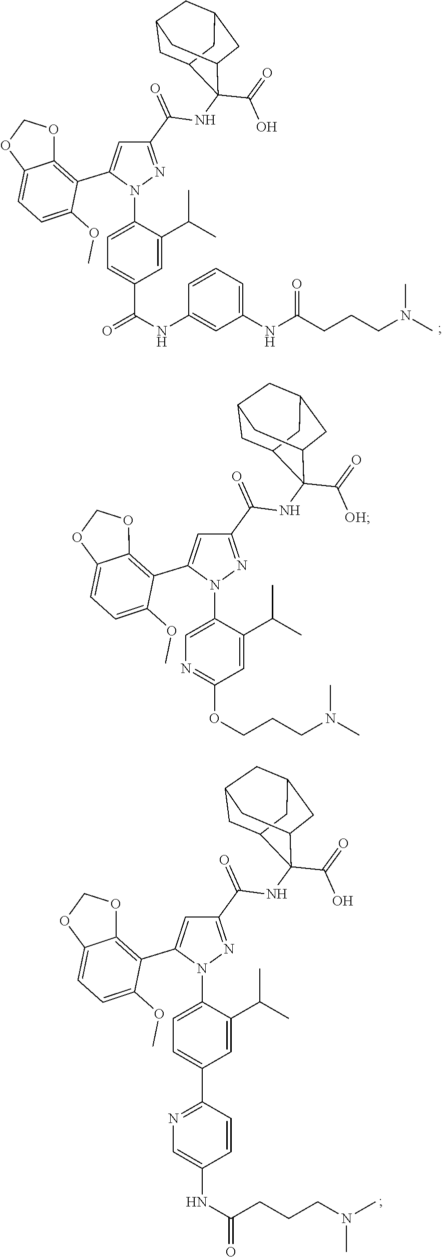

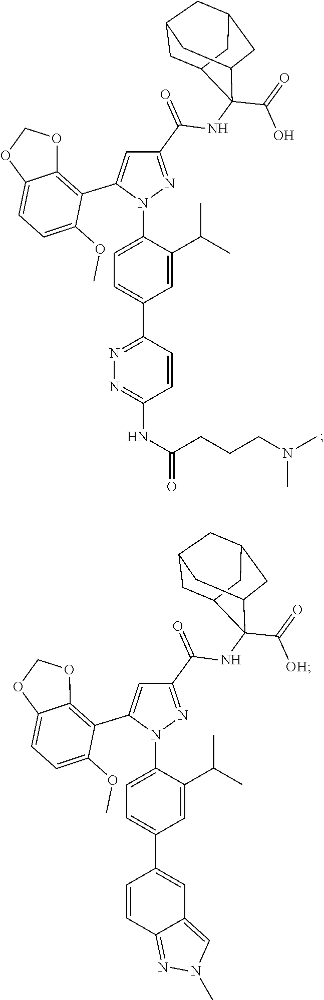 [SMILES: COc1ccc2c(c1-c1cc(C(=O)NC3(C(=O)O)C4CC5CC(C4)CC3C5)nn1-c1ccc(-c3ccc(NC(=O)CCCN(C)C)nn3)cc1C(C)C)OCO2 ; COc1ccc2c(c1-c1cc(C(=O)NC3(C(=O)O)C4CC5CC(C4)CC3C5)nn1-c1ccc(-c3ccc4nn(C)cc4c3)cc1C(C)C)OCO2]

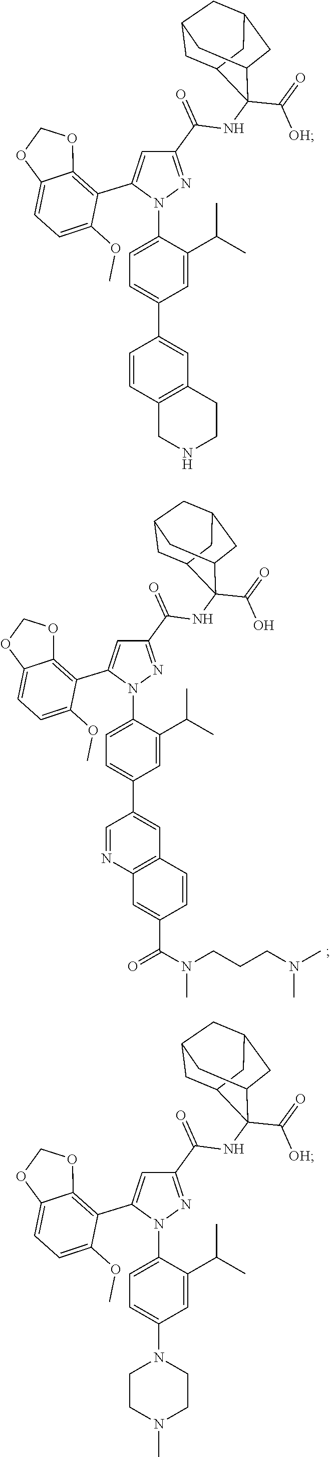

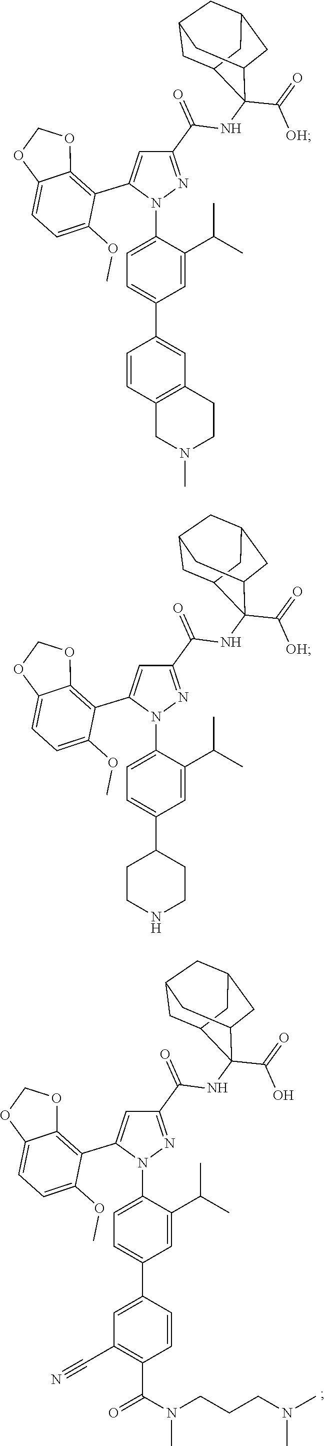

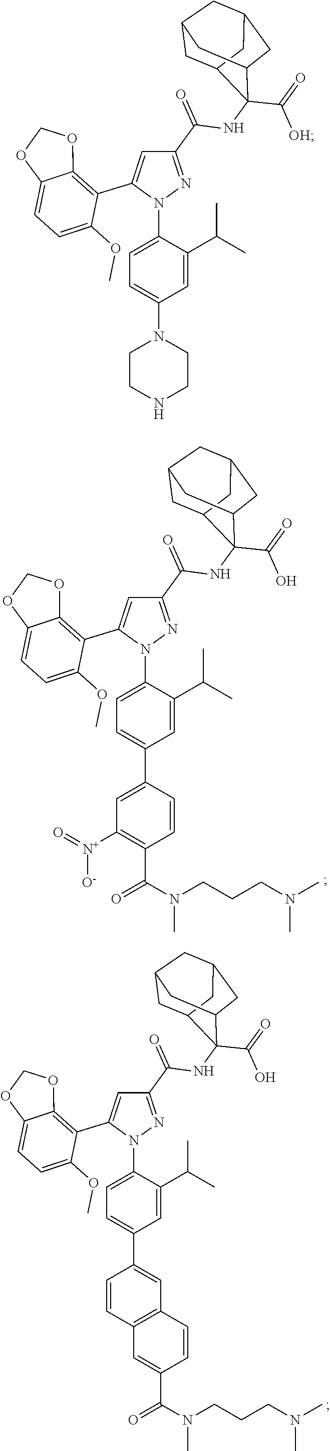

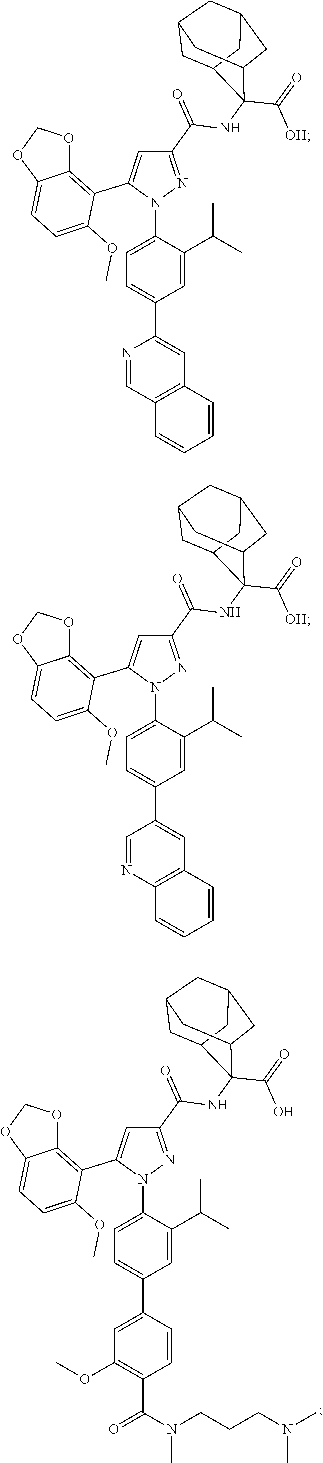

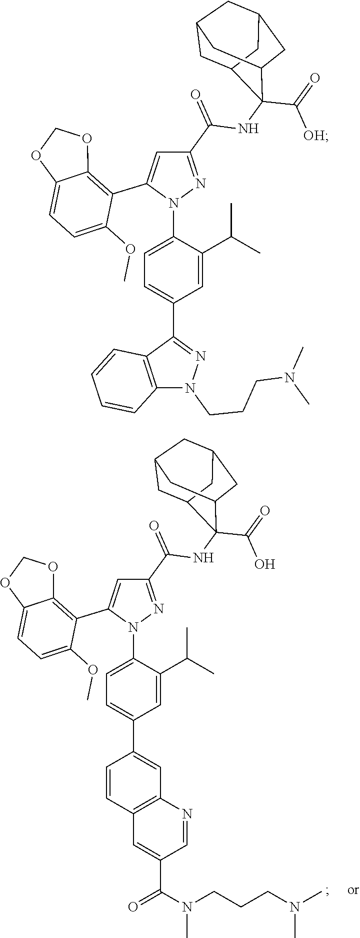

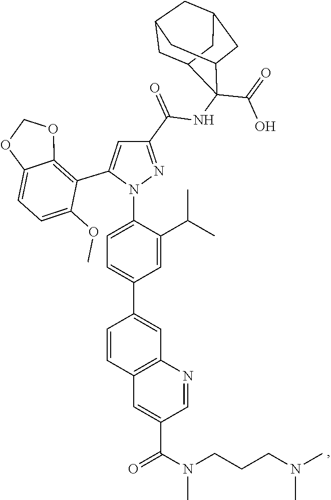 [SMILES: COc1ccc2c(c1-c1cc(C(=O)NC3(C(=O)O)C4CC5CC(C4)CC3C5)nn1-c1ccc(-c3ccc4cc(C(=O)N(C)CCCN(C)C)cnc4c3)cc1C(C)C)OCO2]

or a pharmaceutically acceptable salt thereof.

The present invention also provides a compound of Formula (IV):

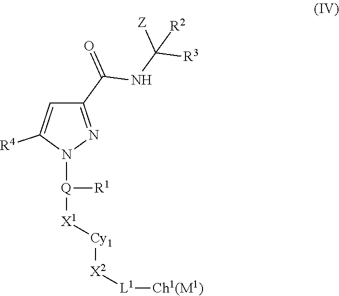

wherein R¹, R², R³, Z, R⁴, Q, X¹, Cy¹, and X² are as defined above; each Y¹ is independently a bond or —NR^{y1}—; Y² is —NR^{y2}R^{y3} or a 5- or 6-membered heterocycle that is pyrrolidine, piperazine, or piperidine, where the 5- or 6-membered heterocycle is optionally substituted with a C₁-C₆ alkyl; R^{y1}, R^{y2}, and R^{y3} at each occurrence are independently selected from H and C₁-C₆ alkyl; and q is 0, 1, 2, 3, 4, 5, or 6; or a pharmaceutically acceptable salt thereof.

In an embodiment of a compound of Formula (II), or a pharmaceutically acceptable salt thereof, q is 3; Y¹ at each occurrence is a bond; and Y² is NR^{y2}R^{y3}.

In an embodiment of a compound of Formula (II), or a pharmaceutically acceptable salt thereof, q is 1; Y¹ is a bond; and Y² is a 5- or 6-membered heterocycle that is pyrrolidine, piperazine, or piperidine, where the 5- or 6-membered heterocycle is optionally substituted with a C₁-C₆ alkyl.

In an embodiment of a compound of Formula (II), or a pharmaceutically acceptable salt thereof, q is 6; five occurrences of Y¹ are a bond, and one occurrence of Y¹ is —NR^{y1}—, and Y² is NR^{y2}R^{y3}.

In an embodiment of a compound of Formula (II), or a pharmaceutically acceptable salt thereof, —X²—(CH₂-Y¹)_{q}-CH₂-Y² is

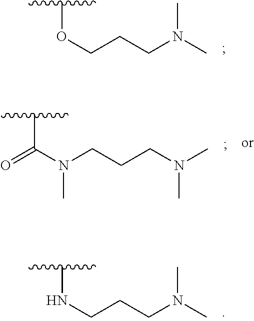

The present invention also provides a compound that is

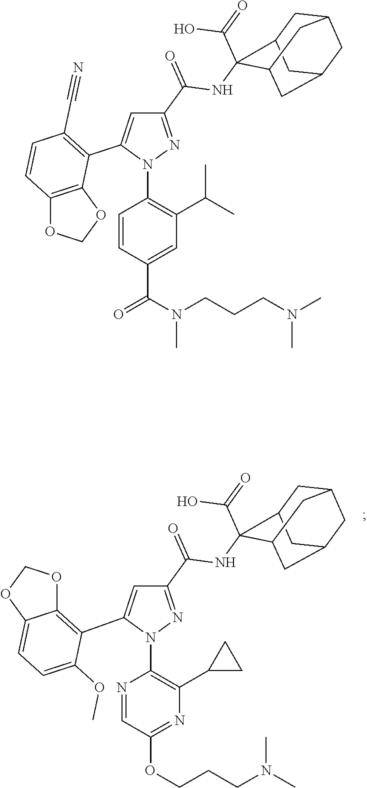 [SMILES: CC(C)c1cc(C(=O)N(C)CCCN(C)C)ccc1-n1nc(C(=O)NC2(C(=O)O)C3CC4CC(C3)CC2C4)cc1-c1c(C#N)ccc2c1OCO2 ; COc1ccc2c(c1-c1cc(C(=O)NC3(C(=O)O)C4CC5CC(C4)CC3C5)nn1-c1ncc(OCCCN(C)C)nc1C1CC1)OCO2]

A compound of Formula (I) is capable of reaction with a number of inorganic and organic acids to form a pharmaceutically acceptable acid addition salt. Examples, reactions and conditions for salt formation are known to the skilled artisan. See for example, P. Stahl, et al., Handbook of Pharmaceutical Salts: Properties, Selection and Use, (VCHA/Wiley-VCH, 2002); S. M. Berge, et al., “Pharmaceutical Salts,” Journal of Pharmaceutical Sciences, Vol. 66, No. 1, January 1977.

It will be understood a compound of Formula (I) may be depicted as a single stereoisomer. As used herein, references to a single stereoisomer are meant to also include stereoisomeric mixtures including the named or depicted compound of Formula (I). Herein, the Cahn-Ingold-Prelog designations of (R)- and (S)- may be used to refer to specific stereoisomers. Specific stereoisomers can be prepared by stereospecific synthesis using enantiomerically pure or enriched starting materials. The specific stereoisomers of either starting materials, intermediates, or racemic mixtures including compounds of Formula (I) can be resolved by techniques well known in the art, such as those found in Stereochemistry of Organic Compounds, E. I. Eliel and S. H. Wilen (Wiley 1994) and Enantiomers, Racemates, and Resolutions, J., Jacques, A Collet, and S. H. Wilen (Wiley 1991), including chromatography on chiral stationary phases, enzymatic resolutions, or fractional crystallization or chromatography of diastereomers formed for that purpose, such as diastereomeric salts. For compounds of Formula (I) having a configuration with all stereocenters shown, “substantially enantiomerically pure” means the isomeric purity is greater than 90% enantiomeric excess.

In another embodiment a compound of Formula (I)isomeric purity is greater than 95% enantiomeric excess.

In still another embodiment a compound of Formula (I)isomeric purity is greater than 98% enantiomeric excess.

In yet another embodiment a compound of Formula (I)isomeric purity is greater than 99% enantiomeric excess. All stereoisomers of the compounds of Formula (I) are contemplated within the scope of the present invention. The designations “isomer 1” and “isomer 2” refer to the compounds that elute from chiral chromatography first and second, respectively, and if chiral chromatography is initiated early in the synthesis, the same designation is applied to subsequent intermediates and examples.

The compounds of formula (I) or any depicted formulae, or salts thereof, may be prepared by a variety of procedures, some of which are illustrated in the Preparations and Examples below. The specific synthetic steps for each of the routes described may be combined in different ways, or in conjunction with steps from different routes, to prepare compounds or salts of the present invention. The products of each step in the Preparations below can be recovered by conventional methods, including extraction, evaporation, precipitation, chromatography, filtration, trituration, and crystallization.

### Synthetic Methods

Compounds of the present invention can be synthesized by following the steps outlined in General Schemes 1, 2 and 3. Starting materials are either commercially available, or made by known procedures in the art or as illustrated below.

Compounds of intermediate (6) may be prepared from intermediate (1) as illustrated in Scheme 1, wherein R¹, R², R³, R⁴, X¹, Cy¹, X², and L¹ are as previously defined, and Z¹ is halogen, preferably bromo or iodo, and Z² is C₁-C₆ alkyl, preferably ethyl.

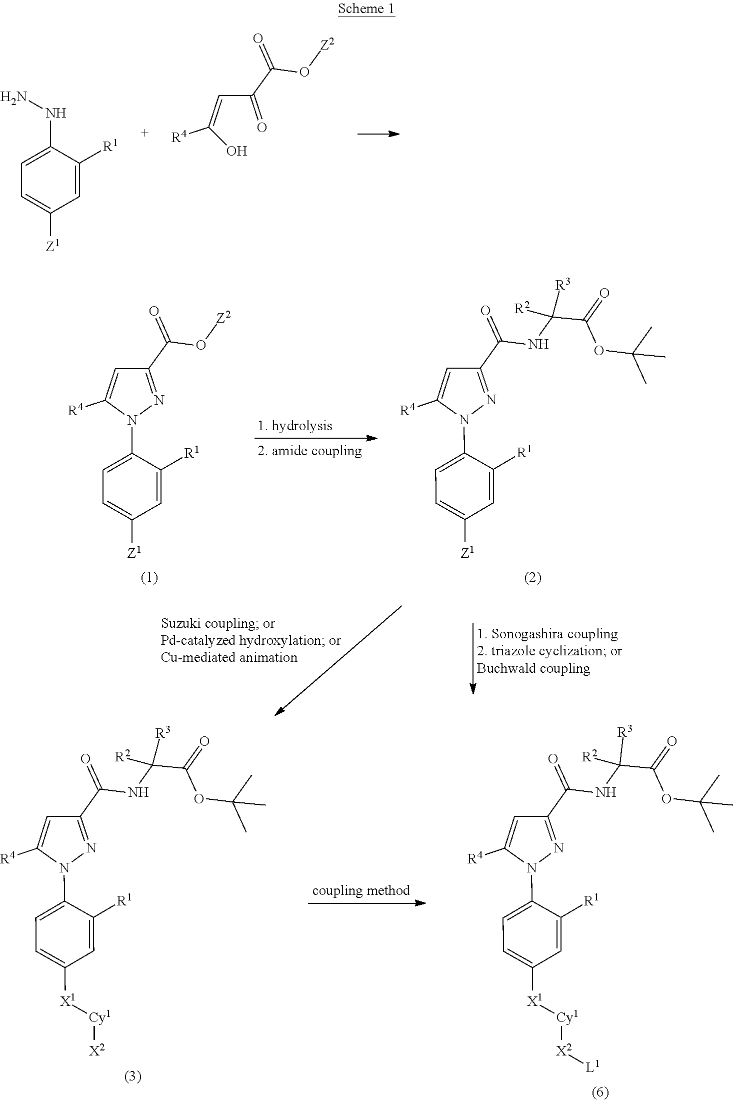

A phenylhydrazine can be reacted with a diketo-ester in a pyrazole cyclization reaction to provide intermediate (1). The phenylhydrazine and diketo-ester are either commercially available or may be prepared by methods well known to the skilled artisan.

The alkyl ester of intermediate (1) can be hydrolyzed using methods well known to the skilled artisan, such as LiOH in a solvent mixture of MeOH and H₂O. The resultant carboxylic acid can be coupled with an appropriate amino acid in a suitable solvent in the presence of a coupling agent and a suitable base to afford intermediate (2). A representative amino acid for this coupling is tert-butyl 2-aminoadamantane-2-carboxylate. Suitable coupling agents include TCFH and HATU. Suitable solvents include DMF and ACN. Suitable bases include DIEA and imidazole.

Intermediate (2) may undergo a Suzuki coupling at position Z¹ with an appropriate phenyl dioxaborolane in the presence of an appropriate palladium catalyst in a suitable solvent to provide compounds of intermediate (3). A representative phenyl dioxaborolane useful for this coupling is 4-carboxyphenylboronic acid pinacol ester. The requisite phenyl dioxaborolanes useful for this coupling are either commercially available or may be prepared by methods well known to the skilled artisan. Suitable palladium catalysts are commercially available and include PdCl₂(PPh₃)₂, Pd₂(dba)₃, and Pd(dppf)Cl₂. Suitable solvents include a mixture of dioxane and H₂O.

Alternatively, intermediate (2) may undergo a palladium catalyzed hydroxylation at the Z¹ position to afford a compound of intermediate (3). A suitable hydroxide source is KOH. Suitable palladium catalysts are commercially available and include t-BuBrettPhos Pd G3. Suitable solvents include a mixture of dioxane and H₂O.

Alternatively, intermediate (2) may undergo a copper mediated amination at the Z¹ position to afford a compound of intermediate (3). A suitable amine source is NH₃·H₂O. A suitable copper source is CuI. Suitable solvents include DMSO.

Intermediate (3) can be elaborated to intermediate (6) via attachment of a linker moiety L¹. The linker may be attached, for example, through an amide, amino, ether, carbamate, urea, carbamothioate, or sulfamide group formed using coupling methods well known to the skilled artisan. The requisite linkers are either commercially available or may be prepared by methods well known to the skilled artisan.

Alternatively intermediate (2) can undergo Sonogashira coupling to install an alkyne which upon treatment with a linker (L¹) functionalized azide will undergo a copper mediated 1,3-dipolar cycloaddition to afford triazole compounds of intermediate (6). The requisite functionalized azides useful for this cycloaddition are either commercially available or may be prepared by methods well known to the skilled artisan. Suitable catalyst systems for Sonogashira coupling are commercially available and include PdCl₂(PPh₃)₂ with CuI. Suitable solvents for Sonogashira coupling include toluene. Suitable copper source for dipolar cycloadditions include CuSO₄. Suitable solvent systems for dipolar cycloadditions include a mixture of MeOH, DCM and H₂O.

Alternatively, compounds of intermediate (2) may undergo a Buchwald coupling at the Z¹ position with an appropriate alkylamine linker (L¹) in the presence of an appropriate palladium catalyst and appropriate ligand in a suitable solvent to afford compounds of intermediate (6). The requisite alkylamine linkers useful for this coupling are either commercially available or may be prepared by methods well known to the skilled artisan. Suitable palladium catalysts are commercially available and include PdCl₂(PPh₃)₂, Pd₂(dba)₃, and Pd(dppf)Cl₂. Suitable ligands are commercially available and include XPhos. Suitable solvents include dioxane.

Alternatively, compounds of intermediate (6) may be prepared from intermediate (4) as illustrated in Scheme 2, wherein R¹, R², R³, R⁴, X¹, Cy¹, X², and L¹ are as previously defined, and Z² is C₁-C₆ alkyl, preferably ethyl.

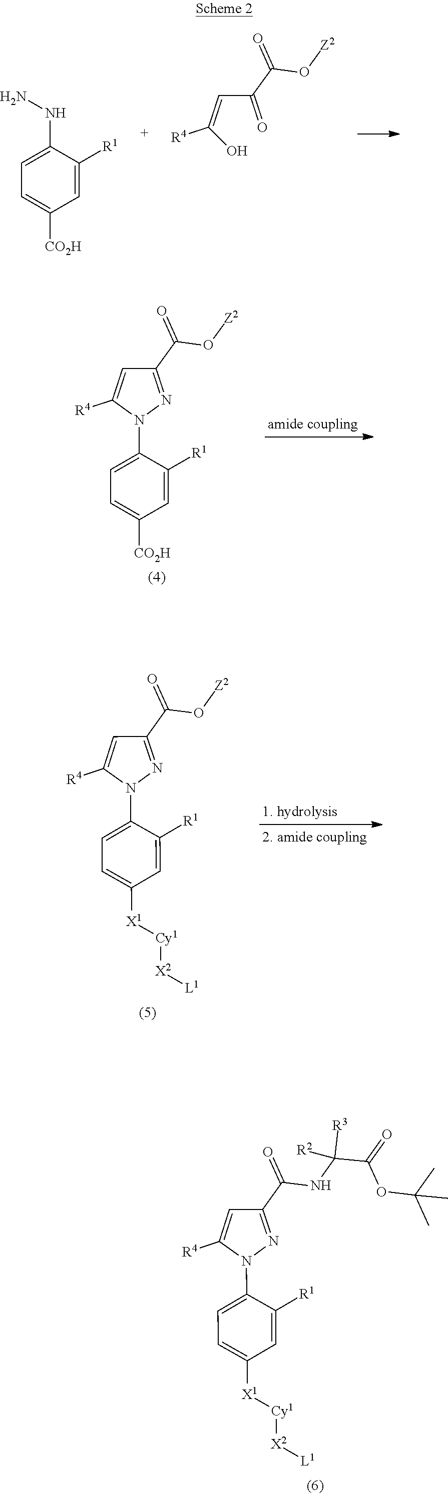

Intermediate (4) containing a carboxylic acid (CO₂H) may undergo an amide coupling with an appropriate alkylamine linker (L¹) in the presence of an amide coupling agent to afford compounds of intermediate (5). The requisite alkylamine linkers useful for this coupling are either commercially available or may be prepared by methods well known to the skilled artisan. Suitable amide coupling agents include T3P. Suitable solvents include DMF.

The alkyl ester of intermediate (5) may be hydrolyzed using methods well known to the skilled artisan, such as LiOH in a solvent mixture of MeOH and H₂O. The resultant carboxylic acid can be coupled with an appropriate amino acid in a suitable solvent in the presence of a coupling agent and a suitable base to afford intermediate (6). A representative amino acid for this coupling is tert-butyl 2-aminoadamantane-2-carboxylate. Suitable coupling agents include TCFH and HATU. Suitable solvents include DMF and ACN. Suitable bases include DIEA and imidazole.

Compounds of Formula (I) may be prepared from intermediate (6) as illustrated in Scheme 3 where R¹, R², R³, R⁴, X¹, Cy¹, X², L¹, Ch¹, and M¹ are as previously defined and Ch¹Pg is Ch¹ with an appropriate protecting group(s).

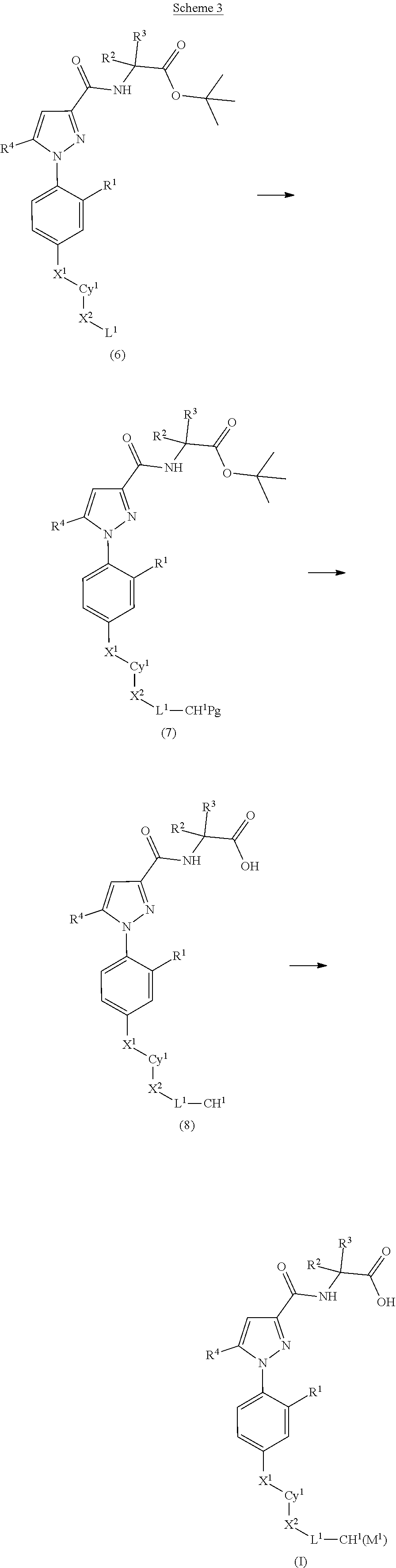

Intermediate (6) is coupled with a suitable chelator to afford intermediate (7). For example, an appropriate protecting group for a chelator may be tert-butyl ester. A representative chelator useful for this coupling may be [4,7-Bis-tert-butoxycarbonylmethyl-10-(2,5-dioxo-pyrrolidin-1-yloxycarbonylmethyl)-1,4,7,10-tetraaza-cyclododec-1-yl]-acetic acid tert-butyl ester. Suitable solvents include DCM and DMF. Suitable bases include pyridine, DIPEA and TEA.

Intermediate (8) may be obtained through global deprotection of intermediate (7) by methods known to the skilled artisan. For example, a tert-butyl ester protecting group may be cleaved by treatment with a suitable acid such as TFA, in a suitable solvent such as DCM.

Compounds of formula (I) may be obtained through metal chelation with intermediate (8). Metal chelation my occur through heating of a solution of intermediate (8) in the presence of a suitable metal. For example, a suitable solution may be pH 5 sodium acetate-acetic acid buffer and a suitable metal may be lutetium (III) chloride.

The skilled artisan will appreciate that the order of the steps described in the proceeding schemes may be varied as necessary or desired to provide useful synthetic intermediates and compounds of Formula (I). For example a linker or part of a linker may be coupled to the chelator prior to coupling with the intermediates.

Certain intermediates described in the following preparations may contain one or more nitrogen and oxygen protecting groups. It is understood that protecting groups may be varied as appreciated by one of skill in the art depending on the particular reaction conditions and the particular transformations to be performed. The protection and deprotection conditions are well known to the skilled artisan and are described in the literature (See for example “Greene's Protective Groups in Organic Synthesis”, Fifth Edition, by Peter G. M. Wuts and Theodora W. Greene, John Wiley and Sons, Inc. 2014).

Examples of known synthetic procedures and methods include those described in general reference texts such as Comprehensive Organic Transformations, VCH Publishers Inc, 1989; Compendium of Organic Synthetic Methods, Volumes 1-10, 197 4-2002, Wiley Interscience; Advanced Organic Chemistry, Reactions Mechanisms, and Structure, 5th Edition, Michael B. Smith and Jerry March, Wiley Interscience, 2001; Advanced Organic Chemistry, 4th Edition, Part B, Reactions and Synthesis, Francis A Carey and Richard J. Sundberg, Kluwer Academic/Plenum Publishers, 2000, etc., and references cited therein.

### Pharmaceutical Compositions

Also provided herein are pharmaceutical compositions including a compound of Formula (I), or a pharmaceutically acceptable salt thereof. A compound of Formula (I), or a pharmaceutically acceptable salt thereof, can be formulated for oral administration in forms such as tablets, capsules (each of which includes sustained release or timed release formulations), pills, powders, granules, elixirs, tinctures, suspensions, syrups and emulsions. A compound of Formula (I), or a pharmaceutically acceptable salt thereof, can also be formulated for intravenous (bolus or in-fusion), intraperitoneal, topical, subcutaneous, intramuscular or transdermal (e.g., patch) administration, all using forms well known to those of ordinary skill in the pharmaceutical arts.

A compound of Formula (I), or a pharmaceutically acceptable salt thereof, or a pharmaceutical composition thereof, may be administered to a subject by any convenient route of administration, whether systemically/peripherally or topically (i.e., at the site of desired action).

Routes of administration include, but are not limited to, oral (e.g. by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

In a preferred embodiment, a compound of Formula (I), or a pharmaceutically acceptable salt thereof, or a pharmaceutical composition thereof, is administered by injection.

A pharmaceutical composition containing a compound of Formula (I), or a pharmaceutically acceptable salt thereof, may be prepared using pharmaceutically acceptable carriers, diluents, or excipients that are compatible with the other additives of the composition or formulation and not deleterious to the patient. Examples of pharmaceutical compositions and processes for their preparation can be found in “Remington: The Science and Practice of Pharmacy”, Loyd, V., et al. Eds., 22nd Ed., Mack Publishing Co., 2012. Non-limiting examples of pharmaceutically acceptable carriers, diluents, and excipients include the following: saline, water, starch, sugars, mannitol, and silica derivatives; binding agents such as carboxymethyl cellulose, alginates, gelatin, and polyvinyl-pyrrolidone; kaolin and bentonite; and polyethyl glycols.

### Therapeutic Uses

Also provided herein are therapies including a compound of Formula (I), or a pharmaceutically acceptable salt thereof, for the treatment of patients with a disease, such as cancer.

Also provided herein is a method of treating cancer, including administering to a patient in need thereof, a therapeutically effective amount of a compound of Formula (I), or a pharmaceutically acceptable salt thereof. The cancer can have one or more cancer cells that overexpress neurotensin receptor 1 (NTSR1). In an embodiment, the cancer is breast cancer, colorectal cancer, endometrial cancer, gastric cancer, lung cancer, pancreatic cancer, prostate cancer, head and neck cancer, non-small-cell lung cancer (NSCLC), pleural mesothelioma, head and neck squamous cell carcinoma (HNSCC), glioma, glioblastoma multiforme (GBM), meningioma, Ewing's sarcoma, gastrointestinal stroma tumors, uterine leiomyoma, cutaneous T-cell lymphoma, small cell lung cancer, or pancreatic ductal adenocarcinoma.

Also provided herein is a method of treating cancer associated with neurotensin receptor 1 (NTSR1) overexpression, including administering to a patient in need thereof, a therapeutically effective amount of a compound of Formula (I), or a pharmaceutically acceptable salt thereof. The status of the cancer as associated with neurotensin receptor 1 (NTSR1) overexpression can be determined by a number of assays known in the art. For example, NTSR 1 overexpression may be detected by immunohistochemical (IHC) analysis of tumor biopsy samples, by enzyme-linked immunosorbent assay (ELISA), or by other suitable protein or gene expression detection methods. If desired, such assays may be utilized to screen patients and confirm that their tumor overexpresses NTSR1, thereby identifying patients who are likely to benefit from treatment with a compound of Formula (I). In an embodiment, the cancer is breast cancer, colorectal cancer, endometrial cancer, gastric cancer, lung cancer, pancreatic cancer, prostate cancer, head and neck cancer, non-small-cell lung cancer (NSCLC), pleural mesothelioma, head and neck squamous cell carcinoma (HNSCC), glioma, glioblastoma multiforme (GBM), meningioma, Ewing's sarcoma, gastrointestinal stroma tumors, uterine leiomyoma, cutaneous T-cell lymphoma, small cell lung cancer, or pancreatic ductal adenocarcinoma.

Also provided herein is a method of treating cancer, comprising administering to a patient in need thereof, a therapeutically effective amount of a compound according to Formula (I), or a pharmaceutically acceptable salt thereof, in which the cancer has one or more cells that overexpress neurotensin receptor 1 (NTSR1). The status of one or more cancer cells can be determined by a number of assays known in the art. In an embodiment, the cancer is breast cancer, colorectal cancer, endometrial cancer, gastric cancer, lung cancer, pancreatic cancer, prostate cancer, head and neck cancer, non-small-cell lung cancer (NSCLC), pleural mesothelioma, head and neck squamous cell carcinoma (HNSCC), glioma, glioblastoma multiforme (GBM), meningioma, Ewing's sarcoma, gastrointestinal stroma tumors, uterine leiomyoma, cutaneous T-cell lymphoma, small cell lung cancer, or pancreatic ductal adenocarcinoma.

Also provided herein is a method of treating cancer, comprising administering to a patient in need thereof, a therapeutically effective amount of a compound according to Formula (I), or a pharmaceutically acceptable salt thereof, wherein the patient has a cancer that was determined to overexpress neurotensin receptor 1 (NTSR1). In an embodiment, the cancer is breast cancer, colorectal cancer, endometrial cancer, gastric cancer, lung cancer, pancreatic cancer, prostate cancer, head and neck cancer, non-small-cell lung cancer (NSCLC), pleural mesothelioma, head and neck squamous cell carcinoma (HNSCC), glioma, glioblastoma multiforme (GBM), meningioma, Ewing's sarcoma, gastrointestinal stroma tumors, uterine leiomyoma, cutaneous T-cell lymphoma, small cell lung cancer, or pancreatic ductal adenocarcinoma.

Further provided herein is a compound or a pharmaceutically acceptable salt thereof according to Formula (I) for use in therapy. In an embodiment, the compound or a pharmaceutically acceptable salt thereof, is for use in treating cancer. The cancer can have one or more cancer cells that overexpress neurotensin receptor 1 (NTSR1). In an embodiment, the cancer is breast cancer, colorectal cancer, endometrial cancer, gastric cancer, lung cancer, pancreatic cancer, prostate cancer, head and neck cancer, non-small-cell lung cancer (NSCLC), pleural mesothelioma, head and neck squamous cell carcinoma (HNSCC), glioma, glioblastoma multiforme (GBM), meningioma, Ewing's sarcoma, gastrointestinal stroma tumors, uterine leiomyoma, cutaneous T-cell lymphoma, small cell lung cancer, or pancreatic ductal adenocarcinoma.

The compounds provided herein according to Formula (I), or a pharmaceutically acceptable salt thereof, may also be used in the manufacture of a medicament for treating cancer. The cancer can have one or more cancer cells that overexpress neurotensin receptor 1 (NTSR1). In an embodiment, the cancer is breast cancer, colorectal cancer, endometrial cancer, gastric cancer, lung cancer, pancreatic cancer, prostate cancer, head and neck cancer, non-small-cell lung cancer (NSCLC), pleural mesothelioma, head and neck squamous cell carcinoma (HNSCC), glioma, glioblastoma multiforme (GBM), meningioma, Ewing's sarcoma, gastrointestinal stroma tumors, uterine leiomyoma, cutaneous T-cell lymphoma, small cell lung cancer, or pancreatic ductal adenocarcinoma.

Also provided herein are methods of diagnosing cancer associated with neurotensin receptor 1 (NTSR1) overexpression, including administering to a patient in need thereof, a diagnostically effective amount of a compound of Formula (I), or a pharmaceutically acceptable salt thereof.

Also provided herein are methods of imaging cancer associated with neurotensin receptor 1 (NTSR1) overexpression, including administering to a patient in need thereof, an imaging effective amount of a compound of Formula (I), or a pharmaceutically acceptable salt thereof.

### Exemplary Aspects

Various aspects of the invention are set forth in the following numbered clauses.

Clause 1. A compound of the formula (I), or a pharmaceutically acceptable salt thereof,

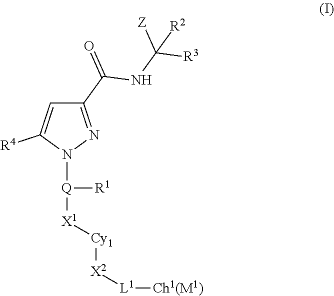

wherein, R¹ is C₁-C₆ alkyl or C₃-C₆ cycloalkyl; Z is —COOH, —C(═O)NH₂, or -tetrazolyl; R² is C₆-C₁₀ cycloalkyl or C₆-C₁₀ cycloalkyl-C₁-C₆ alkyl-, wherein the C₆-C₁₀ cycloalkyl and C₆-C₁₀ cycloalkyl-C₁-C₆ alkyl- are each optionally substituted with 1-3 halogen; R³ is H; or R² and R³ together with the carbon atom to which they are attached form a C₆-C₁₀ cycloalkyl optionally substituted with 1-3 halogen; R⁴ is a group of the formula:

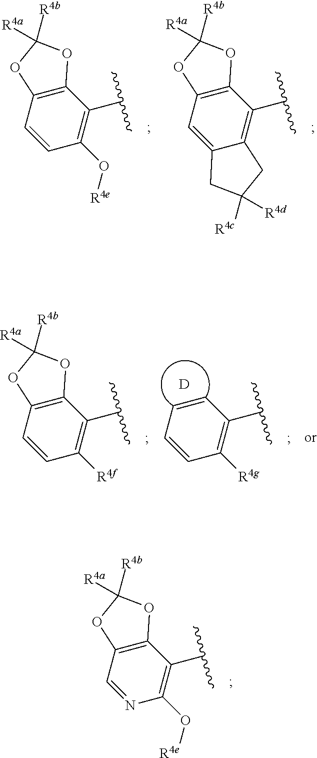

R^{4a}, R^{4b}, R^{4c}, and R^{4d} are each independently H or halogen; R^{4e} is hydrogen, methyl, trifluoromethyl, or methoxymethyl; R^{4f} is cyano or fluoro; R^{4g} is hydrogen; D is a 5- to 6-membered heteroaryl containing 1-3 heteroatoms independently selected from N, O, and S, optionally substituted with 1-3 groups independently selected from halo, trifluoromethyl, hydroxy, cyano, nitro, C₁-C₆ alkyl, C₁-C₆ alkoxy, C₁-C₄ alkylamino, or C₁-C₄ dialkylamino; Q is a 6-membered aryl or heteroaryl ring containing 0-3 ring heteroatoms independently selected from N, O, and S, the remaining ring atoms being carbon; X¹ is a bond, —O—, or —NR^{x1}; Cy¹ is a bond, phenyl, or a 4- or 10-membered heterocycle containing 1-3 heteroatoms independently selected from N, O, and S, which may be monocyclic, fused polycyclic, bridged, or spirocyclic ring system, each phenyl or heterocycle optionally substituted with 1-3 substituents independently selected from halo, trifluoromethyl, trifluoromethoxy, hydroxy, cyano, nitro, C₁-C₆ alkyl, C₁-C₆ alkoxy, C₁-C₄ alkylamino, or C₁-C₄ dialkylamino; provided that when Cy¹ is a bond, X¹ is a bond; X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x1}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}; R^{x1} is H or C₁-C₃ alkyl; R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl; L¹ is a linker; Ch¹ is a chelator; and M¹ is absent or is a radionuclide complexed with Ch¹; or a pharmaceutically acceptable salt thereof.

Clause 2. The compound of clause 1, wherein R⁴ is

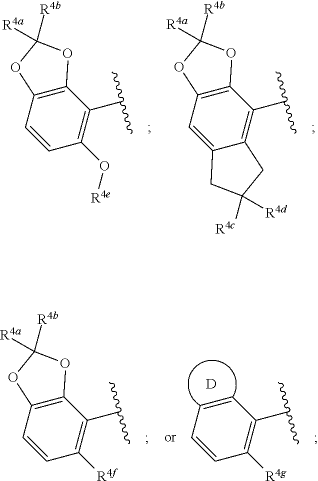

and
Q is

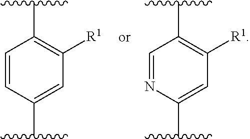

Clause 3. The compound of clause 1, wherein Z is —COOH; R⁴ is

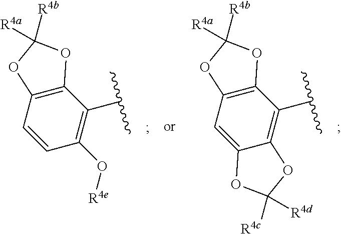

R^{4e} is methyl; Cy¹ is phenyl or a 5- or 6-membered heteroaryl containing 1-3 heteroatoms independently selected from N, O, and S; provided that when Cy¹ is a bond, X¹ is a bond, or a pharmaceutically acceptable salt thereof.

Clause 4. The compound of clause 1-3, or a pharmaceutically acceptable salt thereof, wherein, R¹ is C₁-C₆ alkyl.

Clause 5. The compound of clause 1 or clause 4, or a pharmaceutically acceptable salt thereof, wherein R¹ is isopropyl.

Clause 6. The compound of clause 1 or clause 4, or a pharmaceutically acceptable salt thereof, wherein R¹ is tert-butyl.

Clause 7. The compound of clause 1 or clause 4, or a pharmaceutically acceptable salt thereof, wherein R¹ is C₃-C₆ cycloalkyl.

Clause 8. The compound of clause 7, or a pharmaceutically acceptable salt thereof, wherein R¹ is cyclopropyl.

Clause 9. The compound of clause 7, or a pharmaceutically acceptable salt thereof, wherein R¹ is cyclopentyl.

Clause 10. The compound of any one of clauses 1-9, or a pharmaceutically acceptable salt thereof, wherein R² is C₆-C₁₀ cycloalkyl or C₆-C₁₀ cycloalkyl-C₁-C₆ alkyl-.

Clause 11. The compound of clause 10, or a pharmaceutically acceptable salt thereof, wherein R² is C₆-C₁₀ cycloalkyl.

Clause 12. The compound of clause 11, or a pharmaceutically acceptable salt thereof, wherein R² is adamantyl.

Clause 13. The compound of clause 10, or a pharmaceutically acceptable salt thereof, wherein R² is C₆-C₁₀ cycloalkyl-C₁-C₆ alkyl-.

Clause 14. The compound of clause 13, or a pharmaceutically acceptable salt thereof, wherein R² is adamantyl-CH₂—.

Clause 15. The compound of any one of clauses 1-9, or a pharmaceutically acceptable salt thereof, wherein R² and R³ together with the carbon atom to which they are attached form a C₆-C₁₀ cycloalkyl optionally substituted with 1-3 halogen.

Clause 16. The compound of clause 15, or a pharmaceutically acceptable salt thereof, wherein R² and R³ together with the carbon atom to which they are attached form an adamantyl.

Clause 17. The compound of any one of clauses 1-16, or a pharmaceutically acceptable salt thereof, wherein R⁴ is

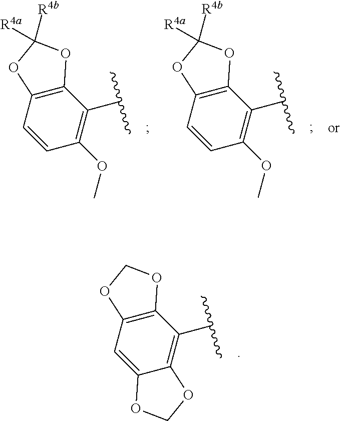

Clause 18. The compound of any one of clauses 1-17, or a pharmaceutically acceptable salt thereof, wherein R⁴ is

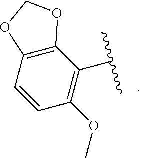 [SMILES: *c1c(OC)ccc2c1OCO2]

Clause 19. The compound of any one of clauses 1, 2 or 4-15, or a pharmaceutically acceptable salt thereof, wherein R⁴ is

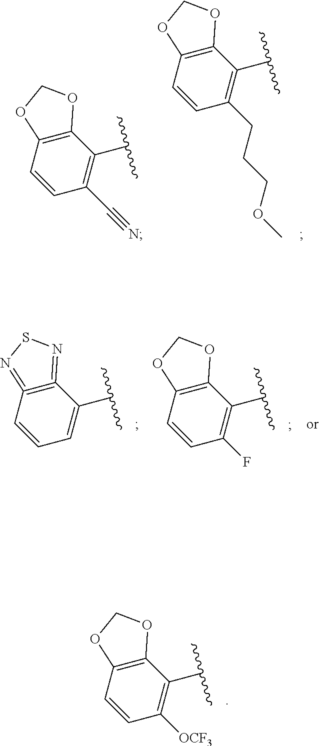

Clause 20. The compound of any one of clauses lor 4-15, or a pharmaceutically acceptable salt thereof, wherein R⁴ is

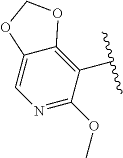 [SMILES: *c1c(OC)ncc2c1OCO2]

Clause 21. The compound of any one of clauses 1-19, or a pharmaceutically acceptable salt thereof, wherein X¹ is a bond.

Clause 22. The compound of any one of clauses 1-19, or a pharmaceutically acceptable salt thereof, wherein X¹ is —O—.

Clause 23. The compound of any one of clauses 1-19, or a pharmaceutically acceptable salt thereof, wherein X¹ is —NH—.

Clause 24. The compound of any one of clauses 1-19, or a pharmaceutically acceptable salt thereof, wherein X¹ is —NCH₃—.

Clause 25. The compound of any one of clauses 1-19, or a pharmaceutically acceptable salt thereof, wherein Cy¹ is a bond.

Clause 26. The compound of any one of clauses 1-24, or a pharmaceutically acceptable salt thereof, wherein Cy¹ is phenyl.

Clause 27. The compound of any one of clauses 1-24, or a pharmaceutically acceptable salt thereof, wherein Cy¹ is 5- or 6-membered heteroaryl containing 1-3 heteroatoms independently selected from N, O, and S.

Clause 28. The compound of clause 27, or a pharmaceutically acceptable salt thereof, wherein Cy¹ is triazolyl.

Clause 29. The compound of any one of clauses 1 or 3-28 or a pharmaceutically acceptable salt thereof, wherein Cy¹ is

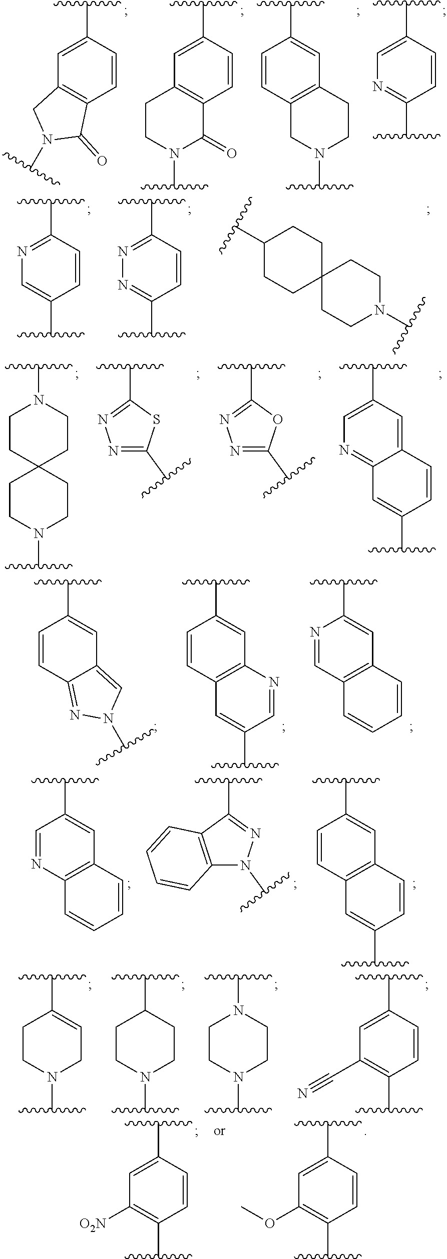

Clause 30. The compound of any one of clauses 1 or 4-28 or a pharmaceutically acceptable salt thereof, wherein Q is:

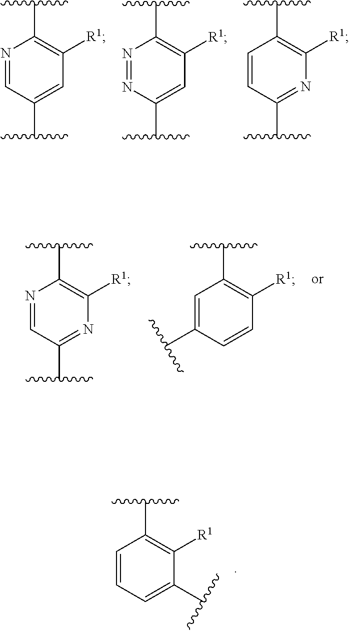

Clause 31. The compound of any one of clauses 1-29, or a pharmaceutically acceptable salt thereof, wherein X² is a bond, —NR^{x2}—, —O—, —NR^{x3}C(O)—, —C(O)NR^{x5}—, —NR^{x7}C(O)NR^{x8}—, —NR^{x11}C(O)O—, —NR^{x13}C(O)S—, or —NR^{x19}S(O)₂NR^{x20}—.

Clause 32. The compound of any one of clauses 1-29, or a pharmaceutically acceptable salt thereof, wherein X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NCH₃—, —NHC(O)O—, —NCH₃C(O)O—, —NHC(O)S—, —NCH₃C(O)S—, —NHS(O)₂NH—, —NHS(O)₂NCH₃—, or —NCH₃S(O)₂NCH₃—.

Clause 33. The compound of any one of clauses 1-30, or a pharmaceutically acceptable salt thereof, wherein X² is a bond, —NH—, —NCH₃—, —O—, —NHC(O)—, —NCH₃C(O)—, —C(O)NCH₃—, —NHC(O)NH—, —NHC(O)NCH₃—, —NCH₃C(O)NCH₃—, —NHC(O)O—, —NCH₃C(O)O—, —NHC(O)S—, or —NHS(O)₂NCH₃—.

Clause 34. The compound of any one of clauses 1-31, or a pharmaceutically acceptable salt thereof, wherein X² is a bond.

Clause 35. The compound of any one of clauses 1-31, or a pharmaceutically acceptable salt thereof, wherein X² is —NH—.

Clause 36. The compound of any one of clauses 1-31, or a pharmaceutically acceptable salt thereof, wherein X² is —NCH₃—.

Clause 37. The compound of any one of clauses 1-31, or a pharmaceutically acceptable salt thereof, wherein X² is —O—.

Clause 38. The compound of any one of clauses 1-31, or a pharmaceutically acceptable salt thereof, wherein X² is —NHC(O)—.

Clause 39. The compound of any one of clauses 1-31, or a pharmaceutically acceptable salt thereof, wherein X² is —NCH₃C(O)—.

Clause 40. The compound of any one of clauses 1-31, or a pharmaceutically acceptable salt thereof, wherein X² is —C(O)NCH₃—.

Clause 41. The compound of any one of clauses 1-31, or a pharmaceutically acceptable salt thereof, wherein X² is —NHC(O)NH—.

Clause 42. The compound of any one of clauses 1-31, or a pharmaceutically acceptable salt thereof, wherein X² is —NHC(O)NCH₃—.

Clause 43. The compound of any one of clauses 1-31, or a pharmaceutically acceptable salt thereof, wherein X² is —NCH₃C(O)NCH₃—.

Clause 44. The compound of any one of clauses 1-31, or a pharmaceutically acceptable salt thereof, wherein X² is —NHC(O)O—.

Clause 45. The compound of any one of clauses 1-31, or a pharmaceutically acceptable salt thereof, wherein X² is —NCH₃C(O)O—.

Clause 46. The compound of any one of clauses 1-31, or a pharmaceutically acceptable salt thereof, wherein X² is —NHC(O)S—.

Clause 47. The compound of any one of clauses 1-31, or a pharmaceutically acceptable salt thereof, wherein X² is —NHS(O)₂NCH₃—.

Clause 48. The compound of any one of clauses 1-47, or a pharmaceutically acceptable salt thereof, wherein L¹ comprises one or more groups independently selected from:

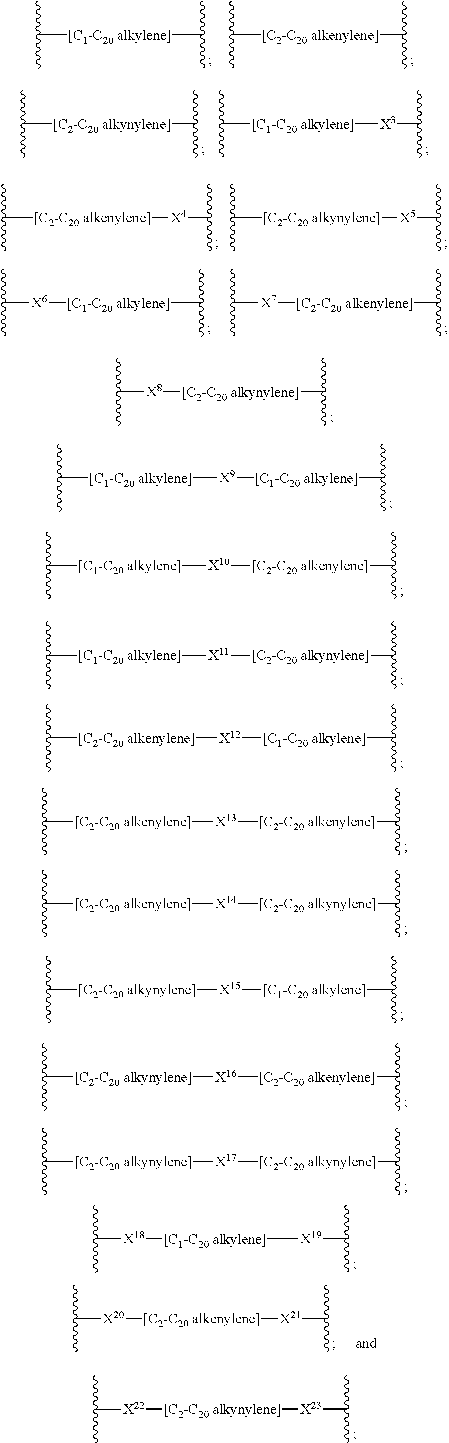

wherein X³, X⁴, X⁵, X⁶, X⁷, X⁸, X⁹, X¹⁰, X¹¹, X¹², X¹³, X¹⁴, X¹⁵, X¹⁶, X¹⁷, X¹⁸, X¹⁹, X²⁰, X²¹, X²², and X²³ at each occurrence are independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}, —C(S)NR^{x25}, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—; R^{x21}, R^{x2}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl; C₁-C₂₀ alkylene, C₂-C₂₀ alkenylene, and C₂-C₂₀ alkynylene at each occurrence are optionally interrupted by one or more groups independently selected from C₃-C₁₀ cycloalkylene, C₄-C₁₀ heterocycle, and C₆-C₁₀ arylene; and C₁-C₂₀ alkylene, C₁-C₂₀ alkenylene, C₁-C₂₀ alkynylene, C₃-C₁₀ cycloalkylene, C₄-C₁₀ heterocycle, and C₆-C₁₀ arylene, at each occurrence are optionally substituted with one or more groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with one or more groups independently selected from halo, —CO₂H, —OH, —NH₂, —NHC₁-C₄ alkyl, and —N(C₁-C₄ alkyl)(C₁-C₄ alkyl).

Clause 49. The compound of clause 48, or a pharmaceutically acceptable salt thereof, wherein L¹ comprises 1-20 groups independently selected from:

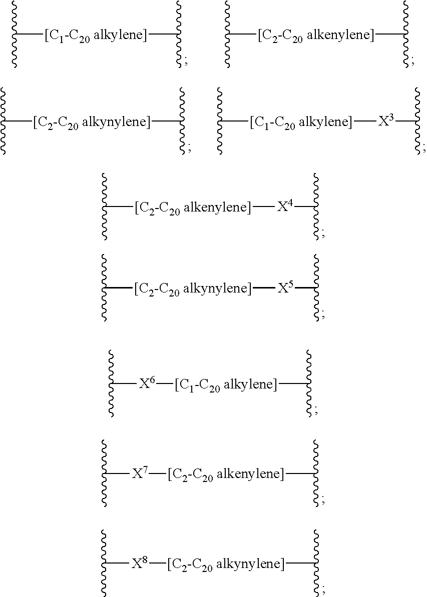

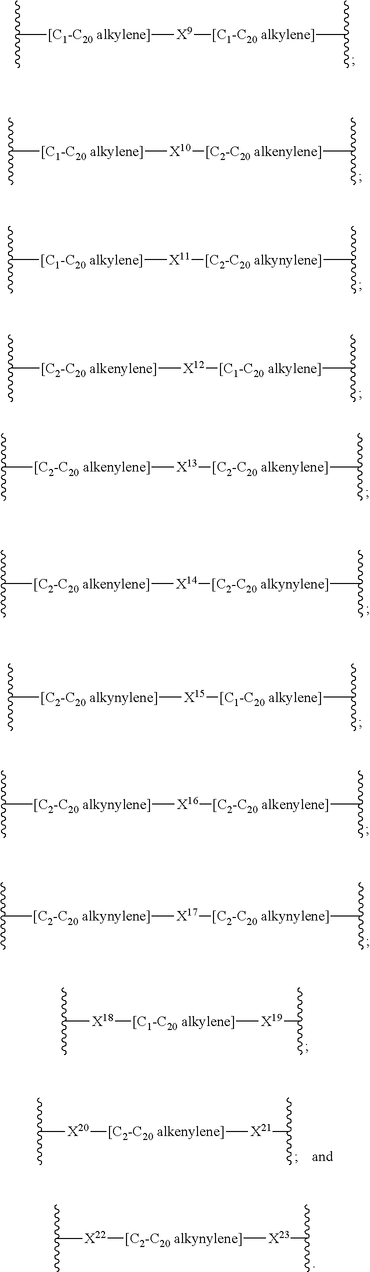

Clause 50. The compound of clause 48, or a pharmaceutically acceptable salt thereof, wherein L¹ comprises 1-5 groups independently selected from:

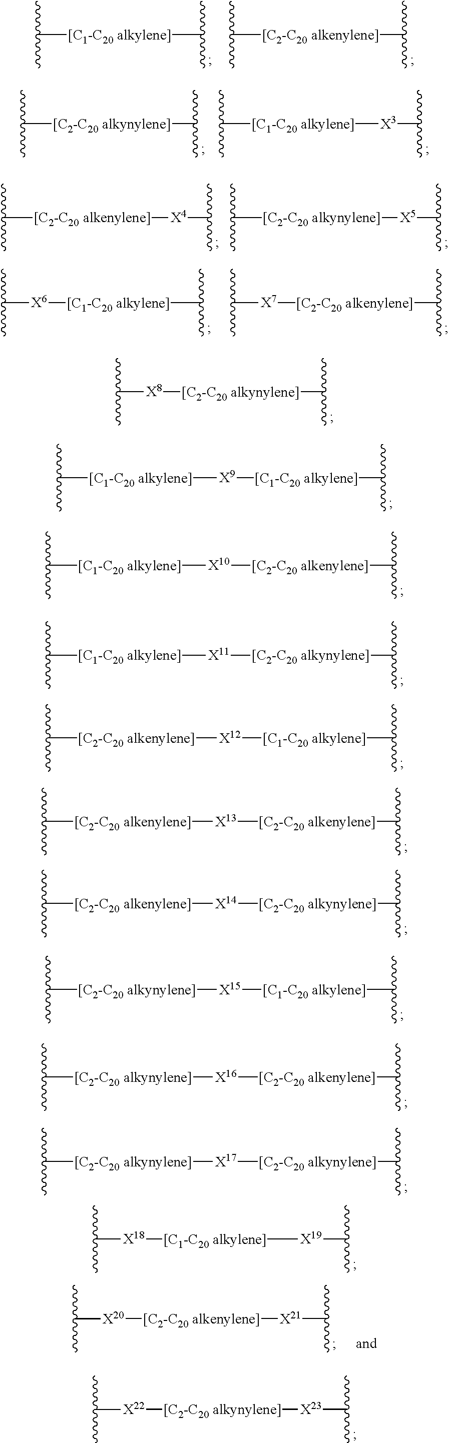

Clause 51. The compound of any one of clauses 1-47, or a pharmaceutically acceptable salt thereof, wherein L¹ comprises 1-20 groups, preferably 1-5 groups, independently selected from

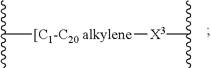

wherein X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}, —OC(S)NR^{x34}—, —SC(O)NR^{x35}—, —S(O)₂NR^{x36}—, —NR^{x37}S(O)₂—, or —NR^{x38}S(O)₂NR^{x39}—; R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl; C₁-C₂₀ alkylene at each occurrence is optionally interrupted by 1-5 groups independently selected from C₃-C₁₀ cycloalkylene, C₄-C₁₀ heterocycle, and, C₆-C₁₀ arylene; and C₁-C₂₀ alkylene, C₃-C₁₀ cycloalkylene, C₄-C₁₀ heterocycle, and, C₆-C₁₀ arylene; at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl).

Clause 52. The compound of any one of clauses 1-47, or a pharmaceutically acceptable salt thereof, wherein L¹ comprises 1-20 groups, preferably 1-5 groups, independently selected from

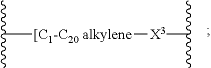

wherein X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—; R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl; C₁-C₃ alkylene at each occurrence is optionally interrupted by 1-5 groups independently selected from C₃-C₁₀ cycloalkylene, C₄-C₁₀ heterocycle, and C₆-C₁₀ arylene; and C₁-C₃ alkylene, C₃-C₁₀ cycloalkylene, C₄-C₁₀ heterocycle, and C₆-C₁₀ arylene; at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl).

Clause 53. The compound of any one of clauses 1-47, or a pharmaceutically acceptable salt thereof, wherein L¹ comprises 1-20 groups, preferably 1-5 groups, independently selected from

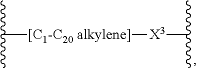

wherein X³ at each occurrence is independently selected from —NR^{x21}, —O—, and —NR^{x22}C(O)—, and R^{x21} and R^{x22} at each occurrence are independently selected from H and C₁-C₆ alkyl.

Clause 54. The compound of any one of clauses 1-47, or a pharmaceutically acceptable salt thereof, wherein L¹ includes 1-20 groups, preferably 1-5 groups, independently selected from

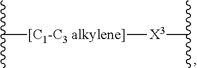

wherein X³ at each occurrence is independently selected from —NR^{x21}—, —O—, and —NR^{x22}C(O)—, and R^{x21} and R^{x22} at each occurrence are independently selected from H and C₁-C₆ alkyl.

Clause 55. The compound of any one of clauses 1-47, or a pharmaceutically acceptable salt thereof, wherein L¹ includes 1-20 groups, preferably 1-5 groups, independently selected from

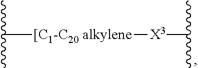

wherein X³ at each occurrence is independently selected from —NH—, —NCH₃—, —O—, and —NHC(O)—.

Clause 56. The compound of any one of clauses 1-47, or a pharmaceutically acceptable salt thereof, wherein L¹ includes 1-20 groups, preferably 1-5 groups, independently selected from

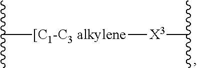

wherein X³ at each occurrence is independently selected from —NH—, —NCH₃—, —O—, and —NHC(O)—.

Clause 57. The compound of any one of clauses 1-47, or a pharmaceutically acceptable salt thereof, wherein L¹ is

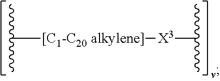

wherein X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}, —SC(O)NR^{x35}—, —S(O)₂NR^{x36}—, —NR^{x37}S(O)₂—, or —NR^{x38}S(O)₂NR^{x39}—; R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl; C₁-C₂₀ alkylene at each occurrence is optionally interrupted by 1-5 groups independently selected from C₃-C₁₀ cycloalkylene, C₄-C₁₀ heterocycle, and C₆-C₁₀ arylene; C₁-C₂₀ alkylene, C₃-C₁₀ cycloalkylene, C₄-C₁₀ heterocycle, and C₆-C₁₀ arylene; at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl); and v is 1, 2, 3, 4, or 5.

Clause 58. The compound of any one of clauses 1-47, or a pharmaceutically acceptable salt thereof, wherein L¹ is

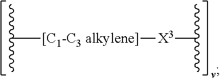

wherein X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—; R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl; C₁-C₃ alkylene at each occurrence is optionally interrupted by 1-5 groups independently selected from C₃-C₁₀ cycloalkylene, C₄-C₁₀, heterocycle, and C₆-C₁₀ arylene; C₁-C₃ alkylene, C₃-C₁₀ cycloalkylene, C₄-C₁₀ heterocycle, and C₆-C₁₀ arylene; at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl); and v is 1, 2, 3, 4, or 5.

Clause 59. The compound of any one of clauses 1-47, or a pharmaceutically acceptable salt thereof, wherein L¹ is

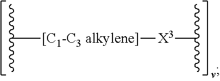

wherein X³ at each occurrence is independently selected from —NH—, —NCH₃—, —O—, and —NHC(O)—, and v is 1, 2, 3, 4, or 5.

Clause 57. The compound of any one of clauses 1-47, or a pharmaceutically acceptable salt thereof, wherein L¹ is selected from:

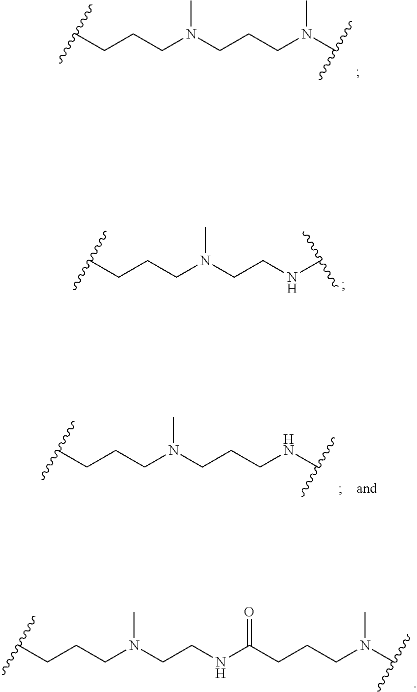

Clause 61. The compound of any one of clauses 1-47, or a pharmaceutically acceptable salt thereof, wherein L¹ is

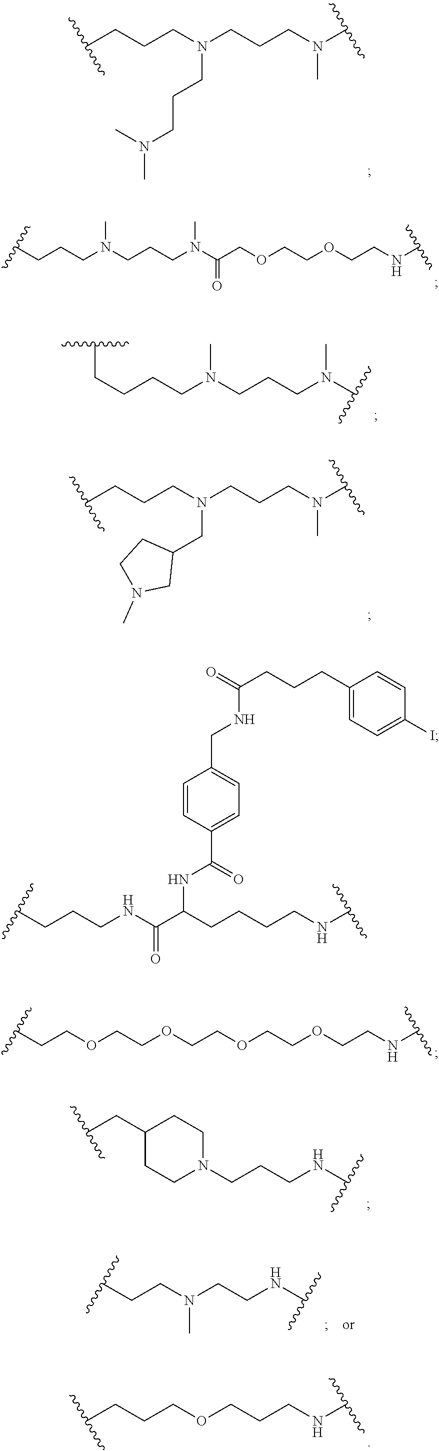

Clause 62. The compound of any one of clauses 1-47, or a pharmaceutically acceptable salt thereof, wherein L¹ is

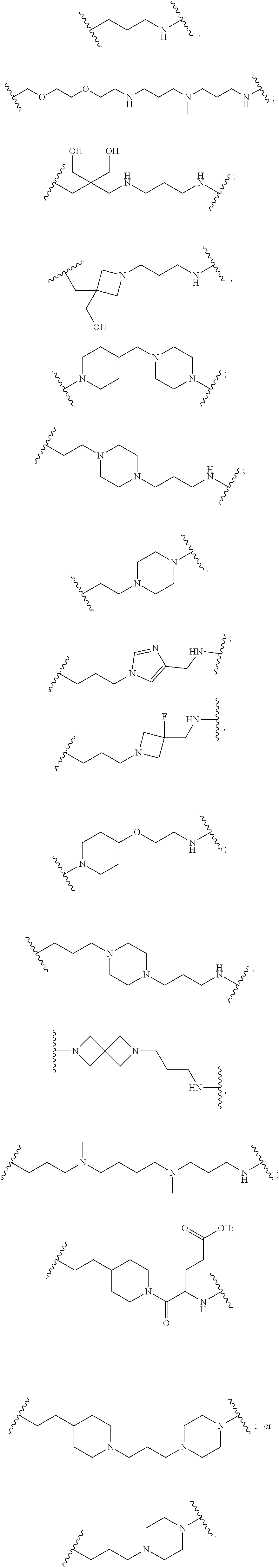

Clause 63. The compound of any one of clauses 1-31, or a pharmaceutically acceptable salt thereof, wherein X²-L¹ is selected from:

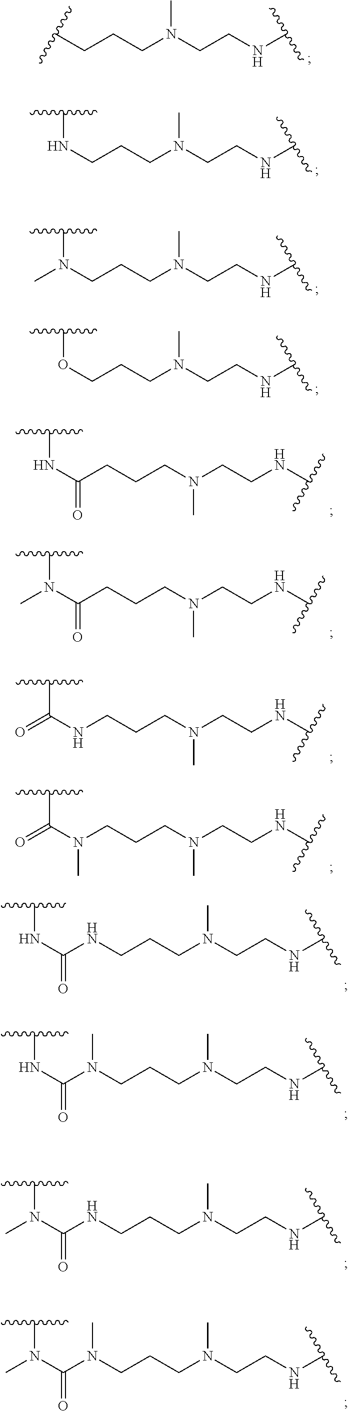

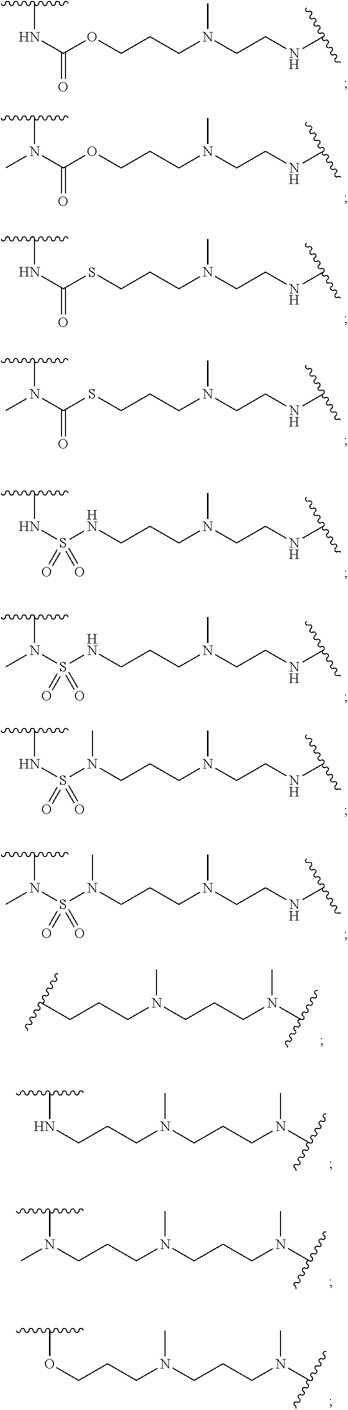

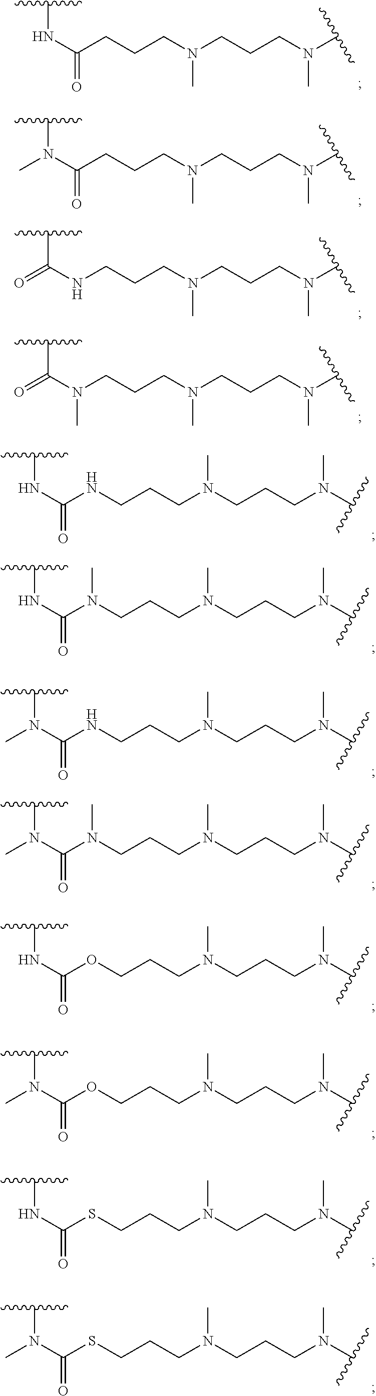

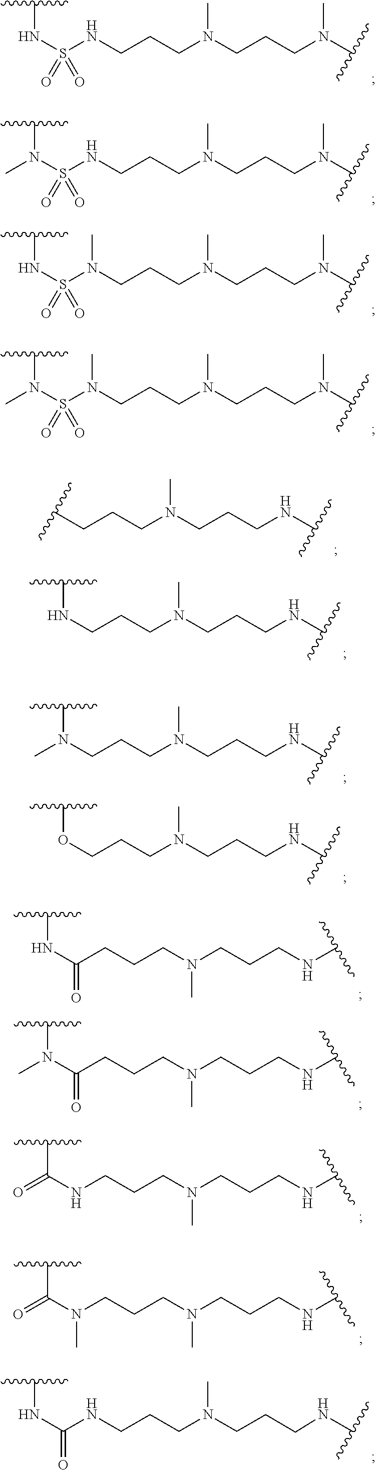

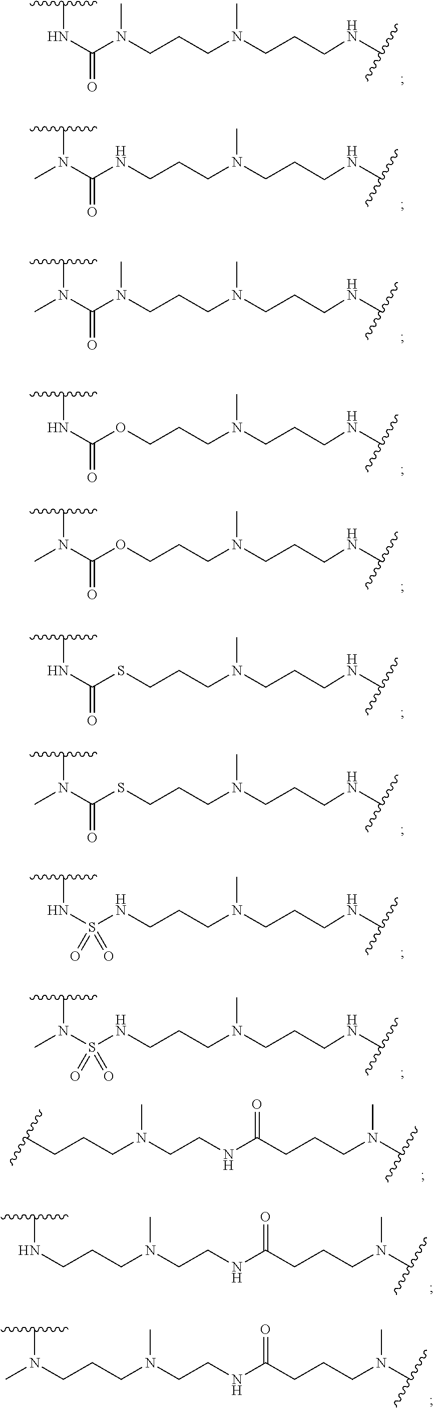

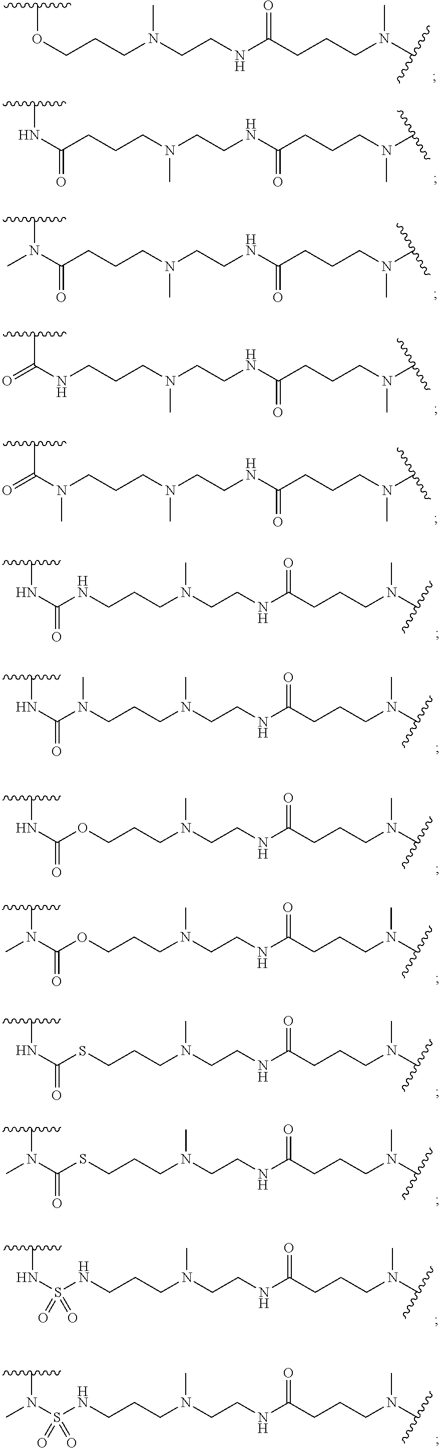

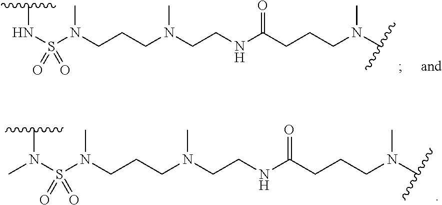 [SMILES: *NS(=O)(=O)N(C)CCCN(C)CCNC(=O)CCCN(*)C ; *N(C)CCCC(=O)NCCN(C)CCCN(C)S(=O)(=O)N(*)C]

Clause 64. The compound of any one of clauses 1-31, or a pharmaceutically acceptable salt thereof, wherein X²-L¹ is selected from:

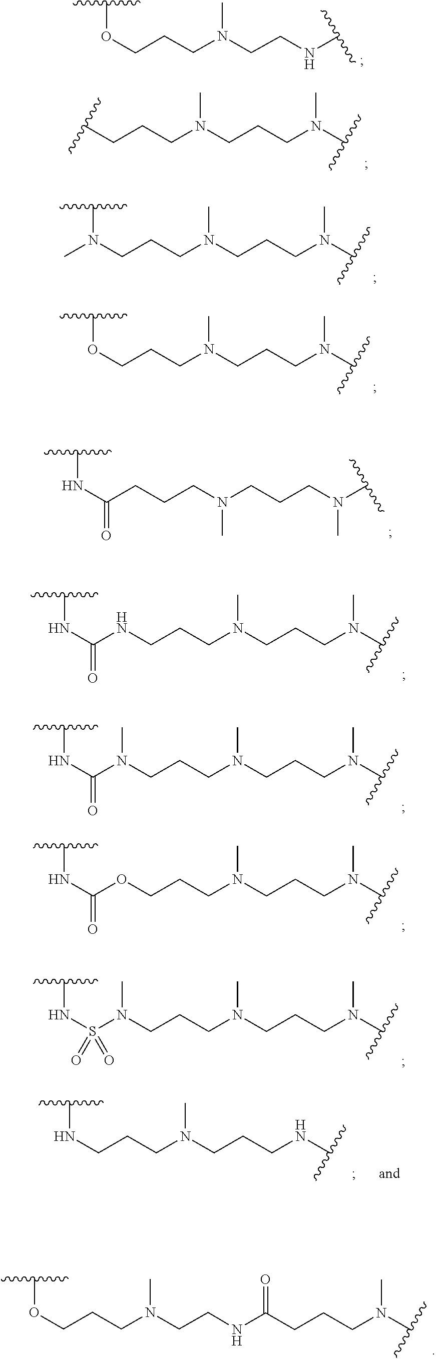

Clause 65. The compound of any one of clauses 1-31, or a pharmaceutically acceptable salt thereof, wherein Ch¹ is derived from 4,7-triazacyclononane (TACN); 1,4,7-triazacyclononane-triacetic acid (NOTA); 1,4,7-triazacyclononane-N-succinic acid-N′,N″-diacetic acid (NOTASA); 1,4,7-triazacyclononane-N-glutamic acid-N′,N″-diacetic acid (NODAGA); 1,4,7-triazacyclononane-N,N′,N″-tris(methylenephosphonic) acid (NOTP); 1,4,7,10-tetraazacyclododecane ([12]aneN4) (cyclen); 1,4,7,10-tetraazacyclotridecane ([13]aneN4); 1,4,7,11-tetraazacyclotetradecane (iso-cyclam); 1,4,7,10-tetraazacyclododecane-1,4,7,10-tetraacetic acid (DOTA); 2-(1,4,7,10-tetraazacyclododecan-1-yl) acetate (DO1A); 2,2′-(1,4,7,10-tetraazacyclododecane-1,7-di-yl) diacetic acid (DO2A); 2,2′,2″-(1,4,7,10-tetraazacyclododecane-1,4,7-triyl)triacetic acid (DO3A); 1,4,7,10-tetraazacyclododecane-1,4,7,10-tetra(methanephosphonic acid) (DOTP); 1,4,7,10-tetraazacyclododecane-1,7-di(methanephosphoric acid) (DO2P); 1,4,7,10-tetraazacyclododecane-1,4,7-tri(methanephosphonic acid) (DO3P); 1,4,7,10-tetraazacyclo-decane-1-glutamic acid-4,7,10-triacetic acid (DOTAGA); 1,4,7,10-tetraazacyclodecane-1-succinic acid-4,7,10-triacetic acid (DOTASA); 1,4,8,11-tetraazacyclotetradecane ([14]aneN4) (cyclam); 1,4,8,12-tetraazacyclopentadecane ([15]aneN4); 1,5,9,13-tetraazacyclohexadecane ([16]aneN4); 1,4-ethano-1,4,8,11-tetraazacyclo-tetradecane (et-cyclam); 1,4,8,11-tetraazacyclotetradecane-1,4,8,11-tetraacetic acid (TETA); 2-(1,4,8,11-tetraazacyclotetradecane-1-yl) acetic acid (TE1A); 2,2′-(1,4,8,11-tetraazacyclotetradecane-1,8-di-yl) diacetic acid (TE2A); 4,11-bis(carboxy methyl)-1,4,8,11-tetraazabicyclo[6.6.2]-hexadecane (CB-TE2A); 3,6,10,13,16,19-hexaazabicyclo[6.6.6]icosane (Sar); 2,2,2,2-(1,4,7,10-tetraazacyclododecane-1,4,7,10-tetraacetamide (TCMC); 1,4,7,10-tetraazacyclododecane-7-acetamide-1,4,10-triacetic acid (PSC); N,N′-bis[(6-carboxy-2-pyridil)methyl]-4,13-diaza-18-crown-6 (macropa), a phthalocyanine, or a porphoryin.

Clause 66. The compound of any one of clauses 1-64, or a pharmaceutically acceptable salt thereof, wherein Ch¹ is an aminopolycarboxylate chelator.

Clause 67. The compound of any one of clauses 1-64, or a pharmaceutically acceptable salt thereof, wherein Ch¹ is a macrocyclic aminopolycarboxylate chelator.

Clause 68. The compound of any one of clauses 1-64, or a pharmaceutically acceptable salt thereof, wherein Ch¹(M¹) is selected from:

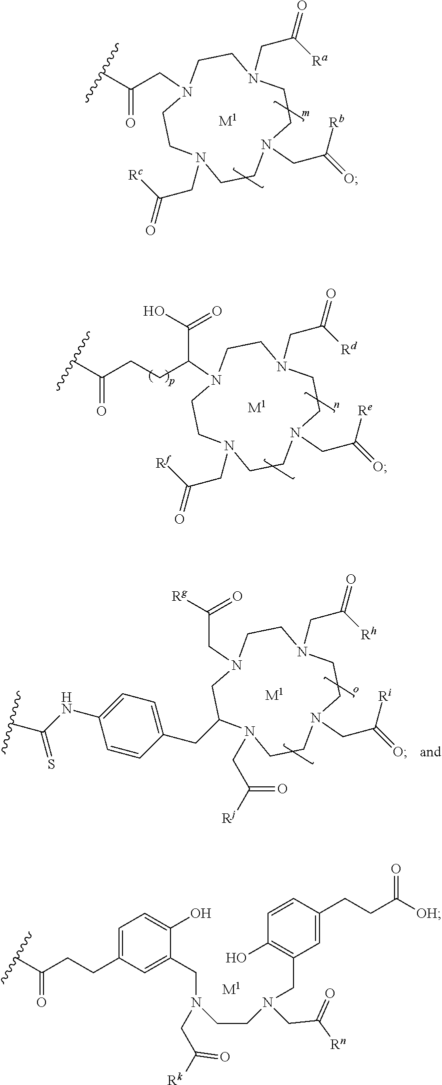

wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2.

Clause 69. The compound of any one of clauses 1-64, or a pharmaceutically acceptable salt thereof, wherein Ch¹(M¹) is selected from:

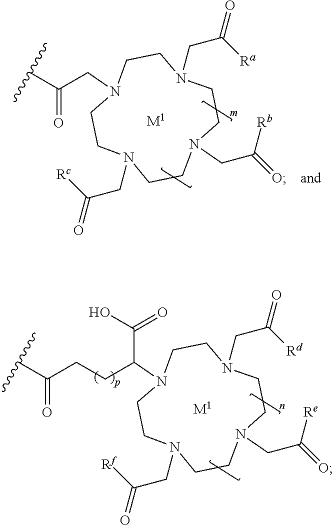

wherein R^{a}, R^{b}, R^{c}, R^{d}, R^{e}, and R^{f} are each independently selected from —OH and —NH₂; m, n, and p are each independently 0, 1, or 2.

Clause 70. The compound of any one of clauses 1-64, or a pharmaceutically acceptable salt thereof, wherein Ch¹(M¹) is selected from:

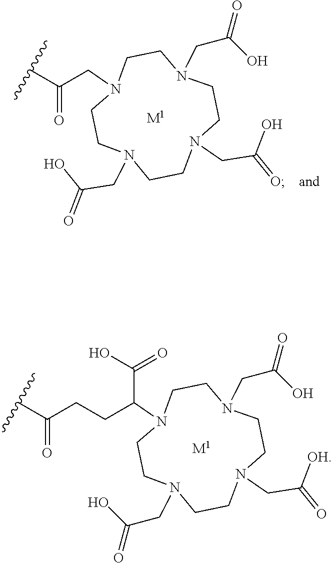

Clause 71. The compound of any one of clauses 1-70, or a pharmaceutically acceptable salt thereof, wherein M¹ is absent.

Clause 72. The compound of any one of clauses 1-70, or a pharmaceutically acceptable salt thereof, wherein M¹ is a radionuclide.

Clause 73. The compound of clause 72, or a pharmaceutically acceptable salt thereof, wherein M¹ is ¹⁷⁷Lu, ²¹²Pb, or ²²⁵Ac.

Clause 74. The compound of clause 72 or clause 73, or a pharmaceutically acceptable salt thereof, wherein M¹ is ¹⁷⁷Lu.

Clause 75. The compound of any one of clauses 1-47, or a pharmaceutically acceptable salt thereof, wherein L¹-Ch¹(M¹) is selected from:

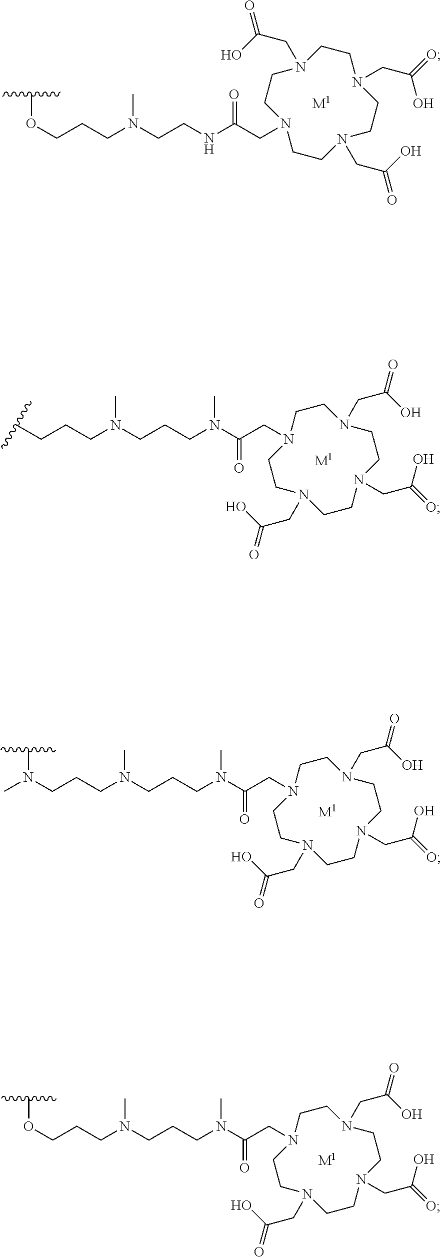

wherein M¹ is a radionuclide, preferably M¹ is ¹⁷⁷Lu, ²¹²Pb, or ²²⁵Ac.

Clause 76. The compound of any one of clauses 1-26, or a pharmaceutically acceptable salt thereof, wherein X²-L¹-Ch¹(M¹) is selected from:

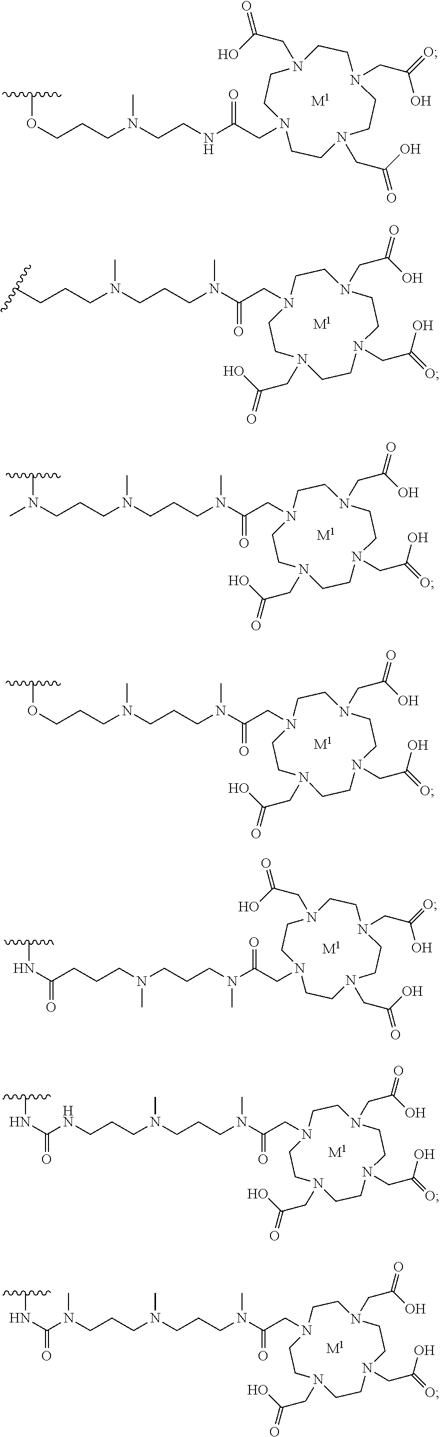

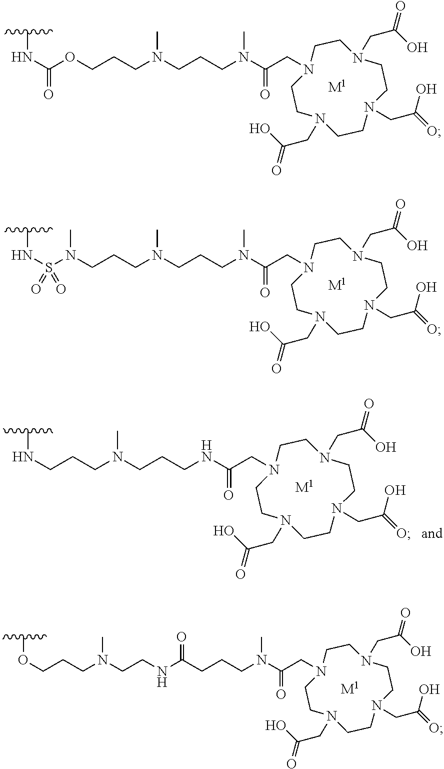

wherein M¹ is a radionuclide, preferably M¹ is ¹⁷⁷Lu, ²¹²Pb, or ²²⁵Ac.

Clause 77. The compound of clause 1, selected from:

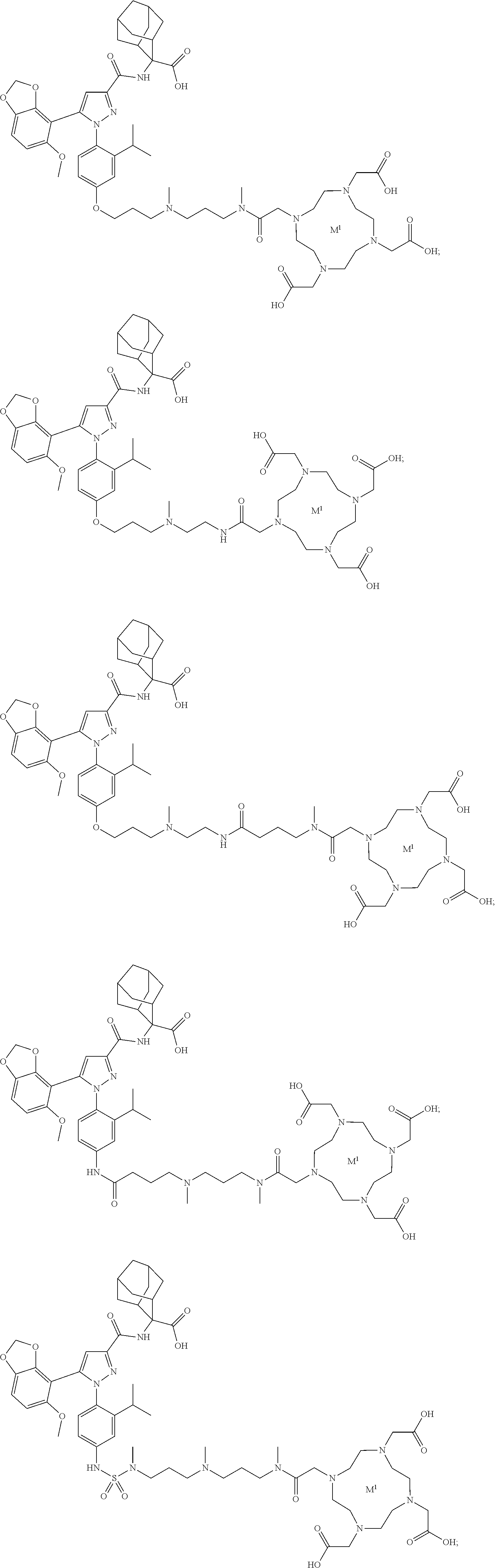

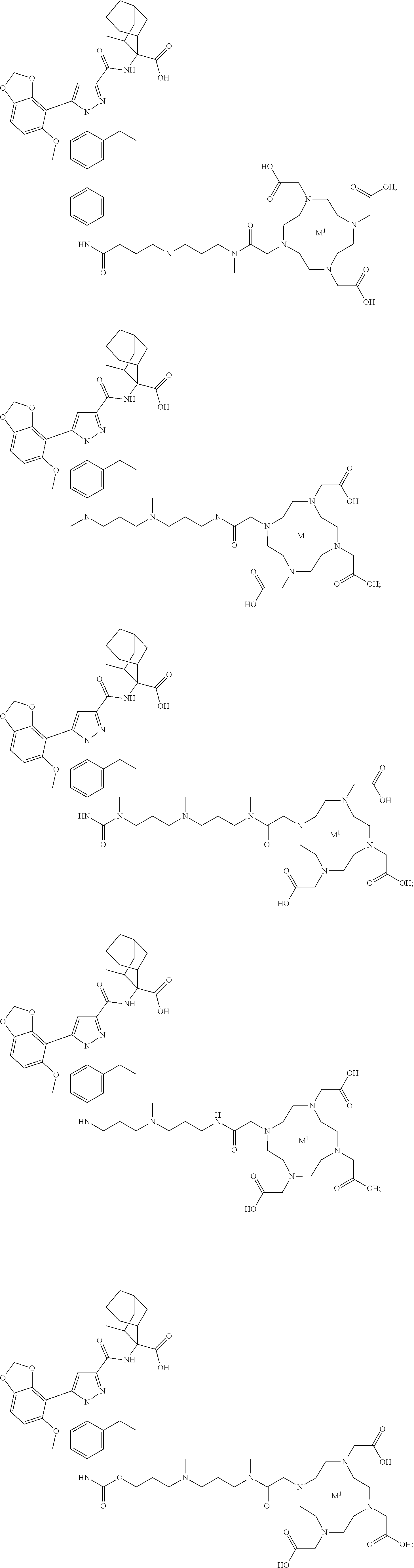

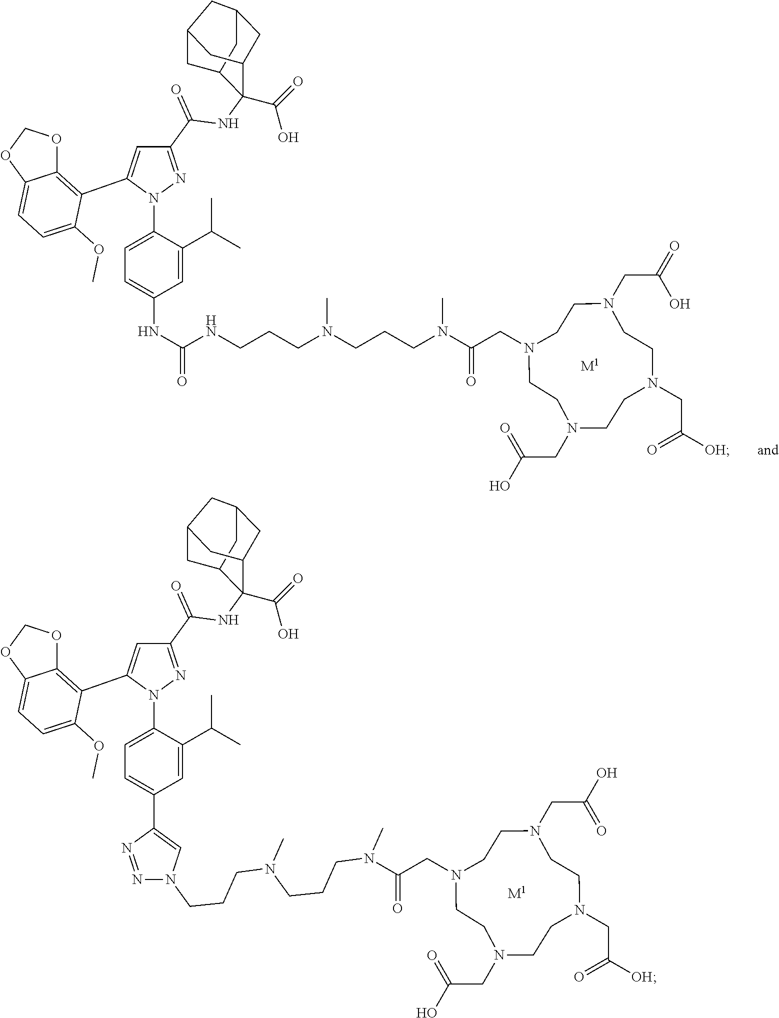 [SMILES: COc1ccc2c(c1-c1cc(C(=O)NC3(C(=O)O)C4CC5CC(C4)CC3C5)nn1-c1ccc(NC(=O)NCCCN(C)CCCN(C)C(=O)CN3CCN(CC(=O)O)CCN(CC(=O)O)CCN(CC(=O)O)CC3)cc1C(C)C)OCO2 ; "M1" ; "and" ; COc1ccc2c(c1-c1cc(C(=O)NC3(C(=O)O)C4CC5CC(C4)CC3C5)nn1-c1ccc(-c3cn(CCCN(C)CCCN(C)C(=O)CN4CCN(CC(=O)O)CCN(CC(=O)O)CCN(CC(=O)O)CC4)nn3)cc1C(C)C)OCO2 ; "M1"]

or a pharmaceutically acceptable salt thereof.

Clause 78. The compound of clause 1, which is

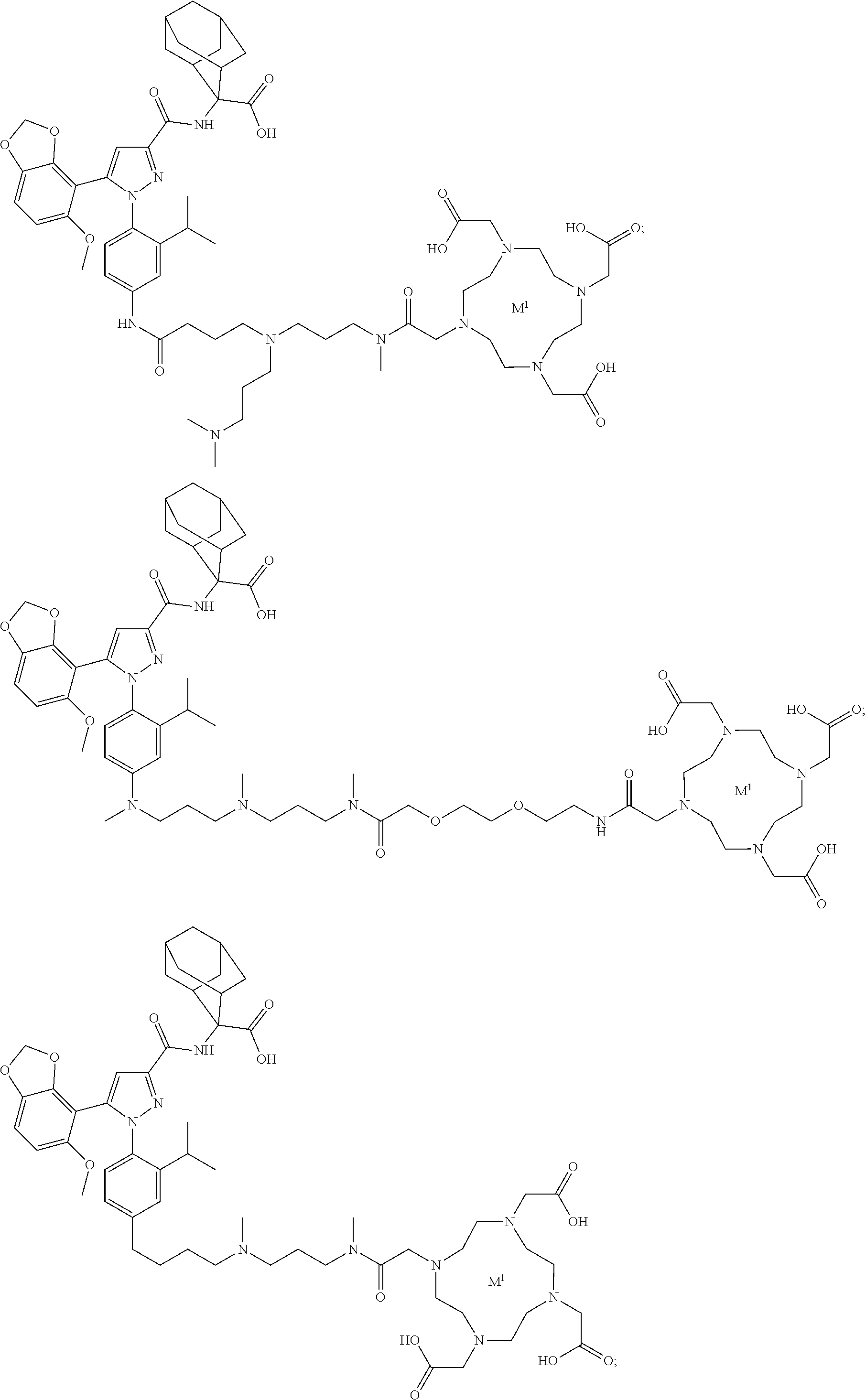

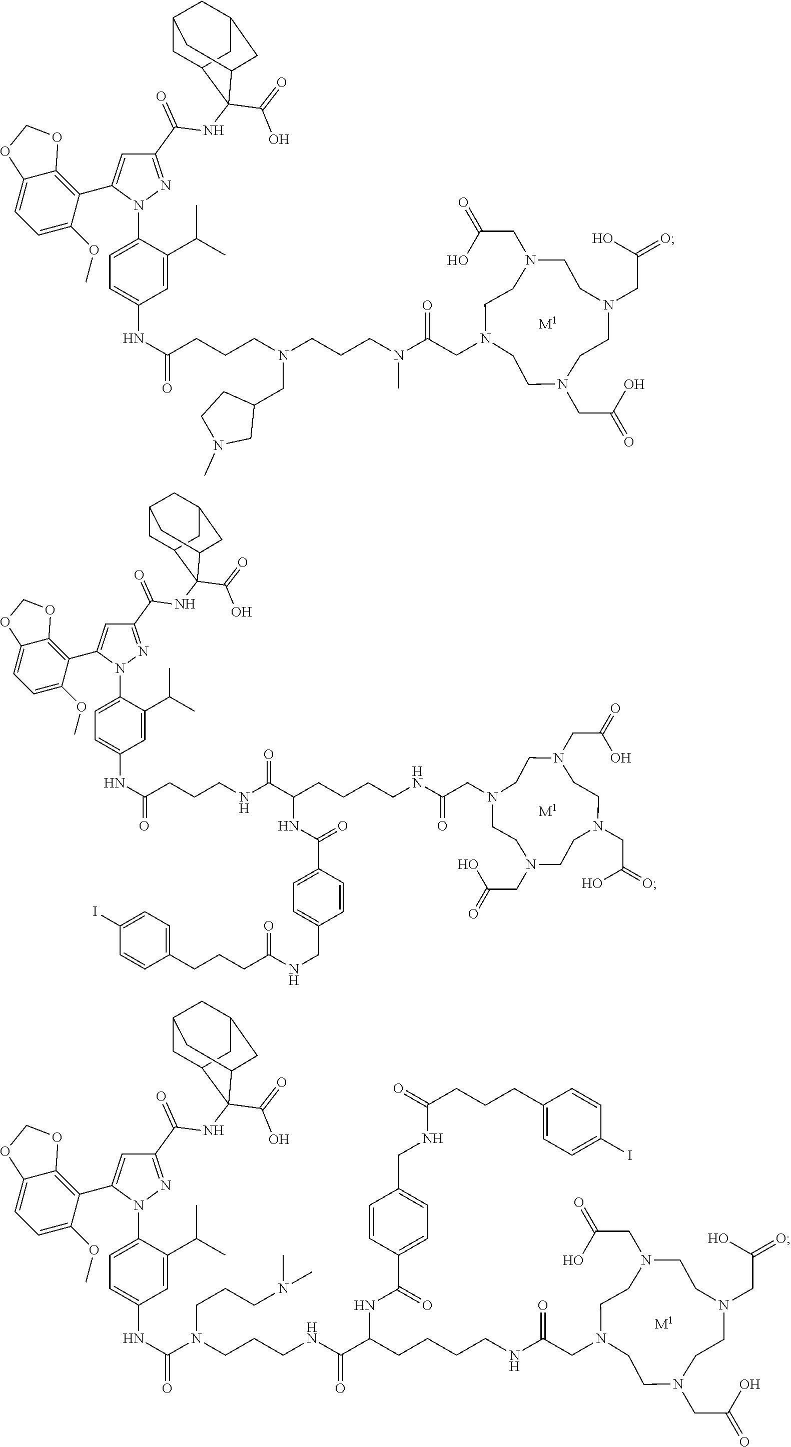

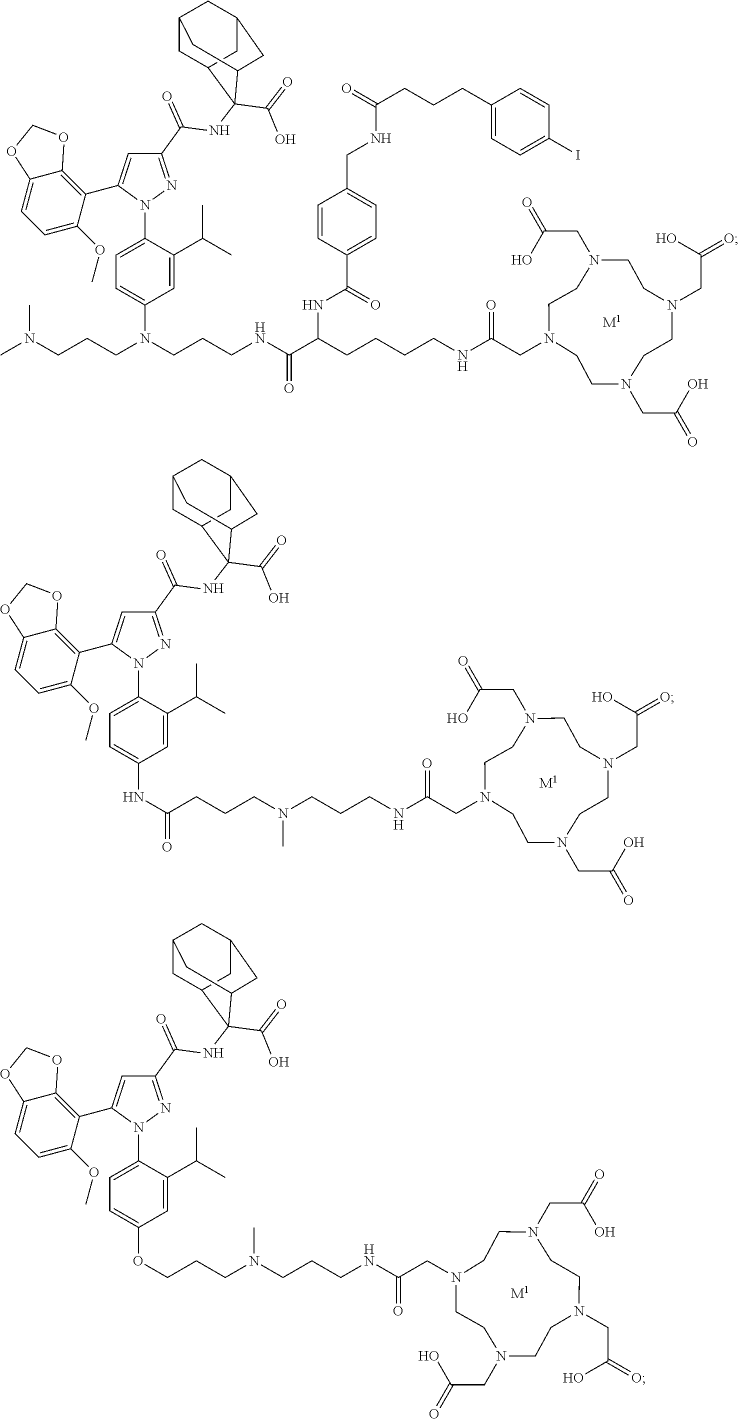

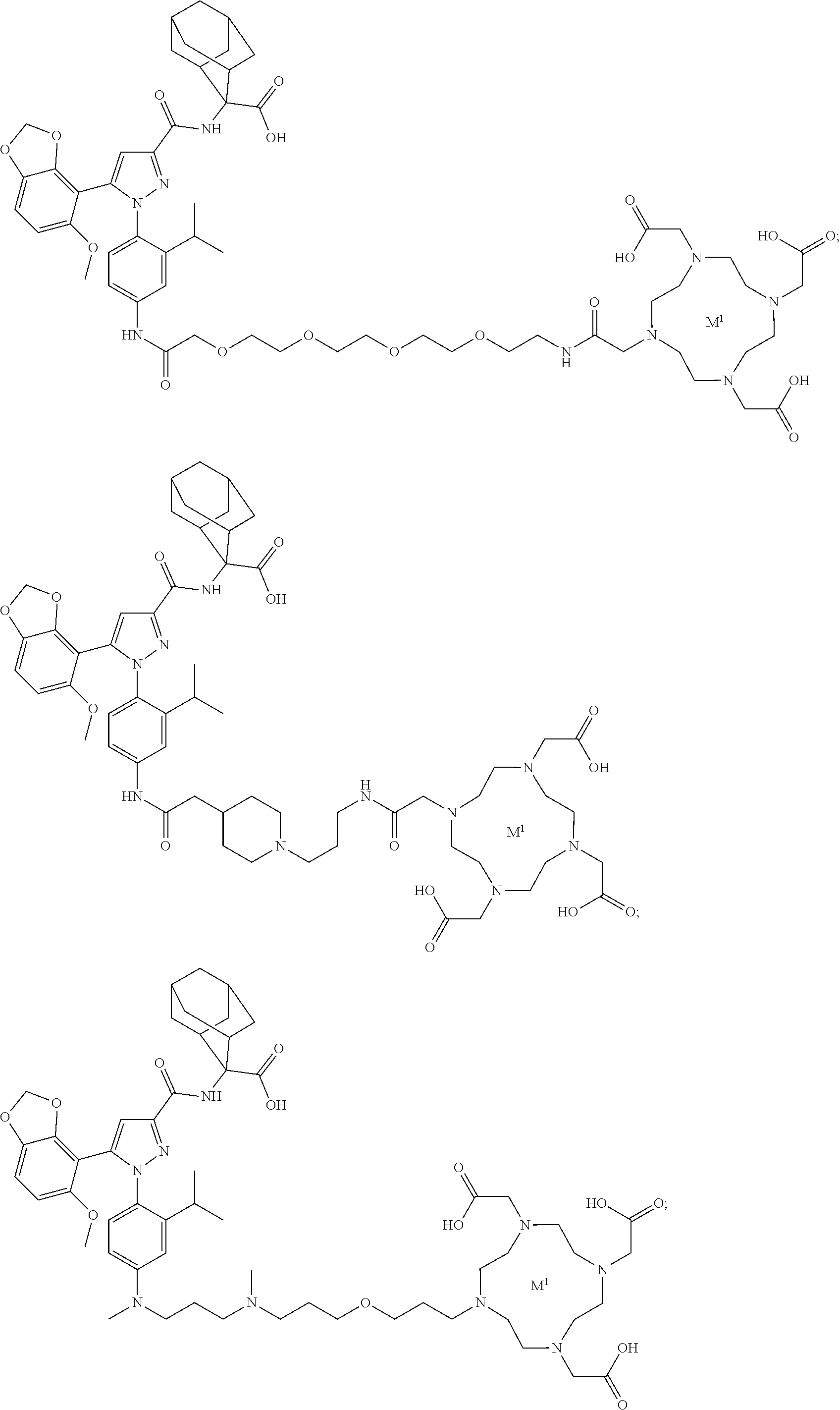

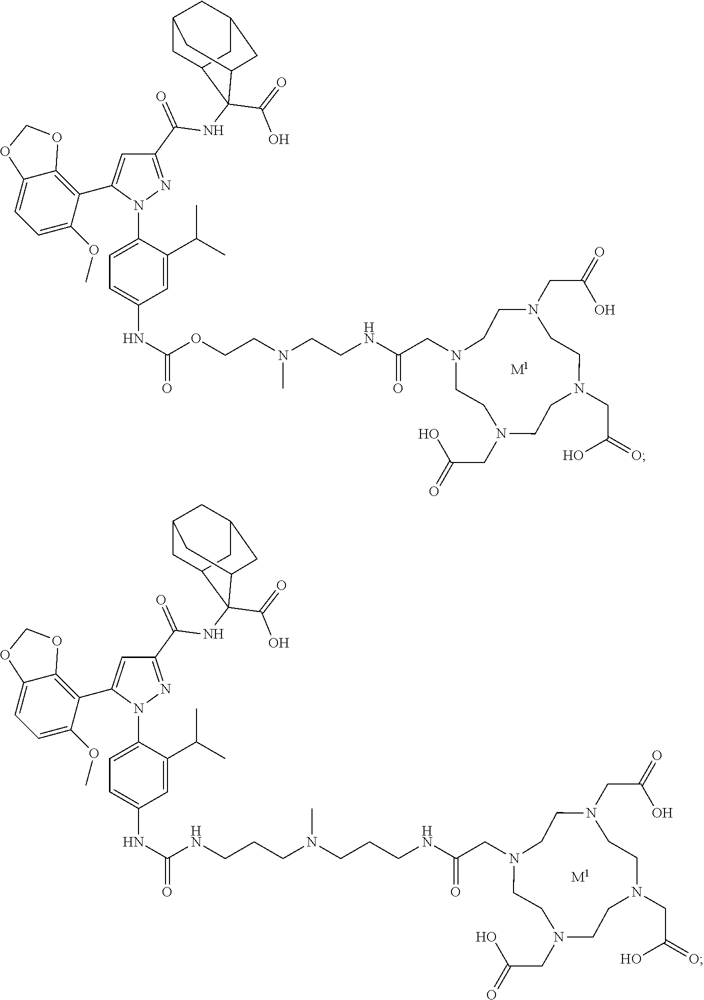

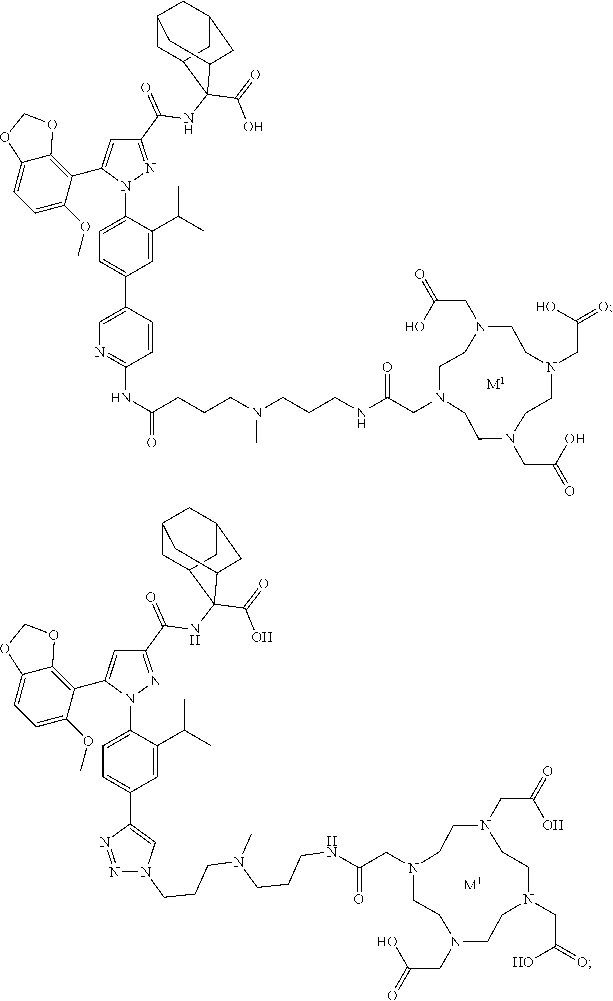

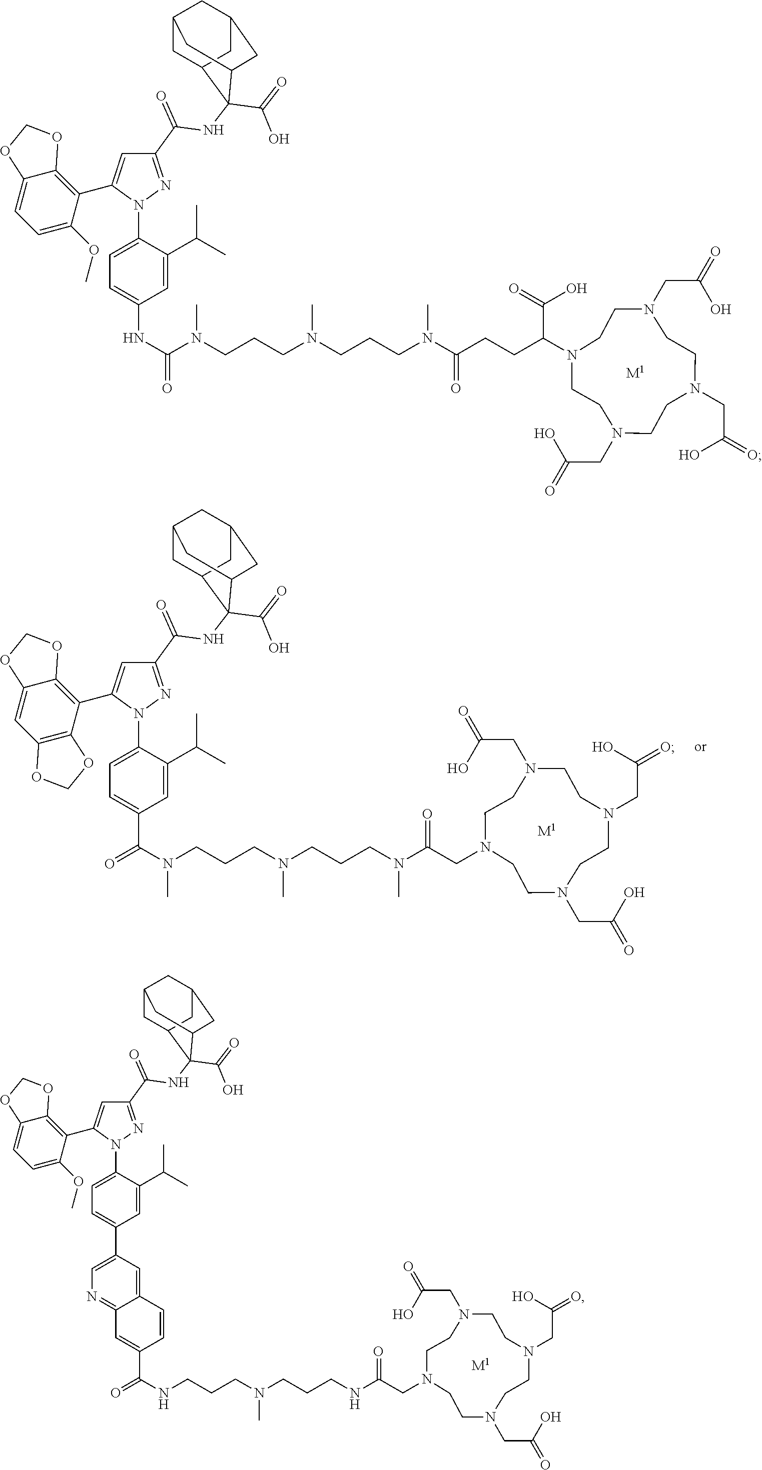

or a pharmaceutically acceptable salt thereof.

Clause 79. The compound of clause 1, selected from:

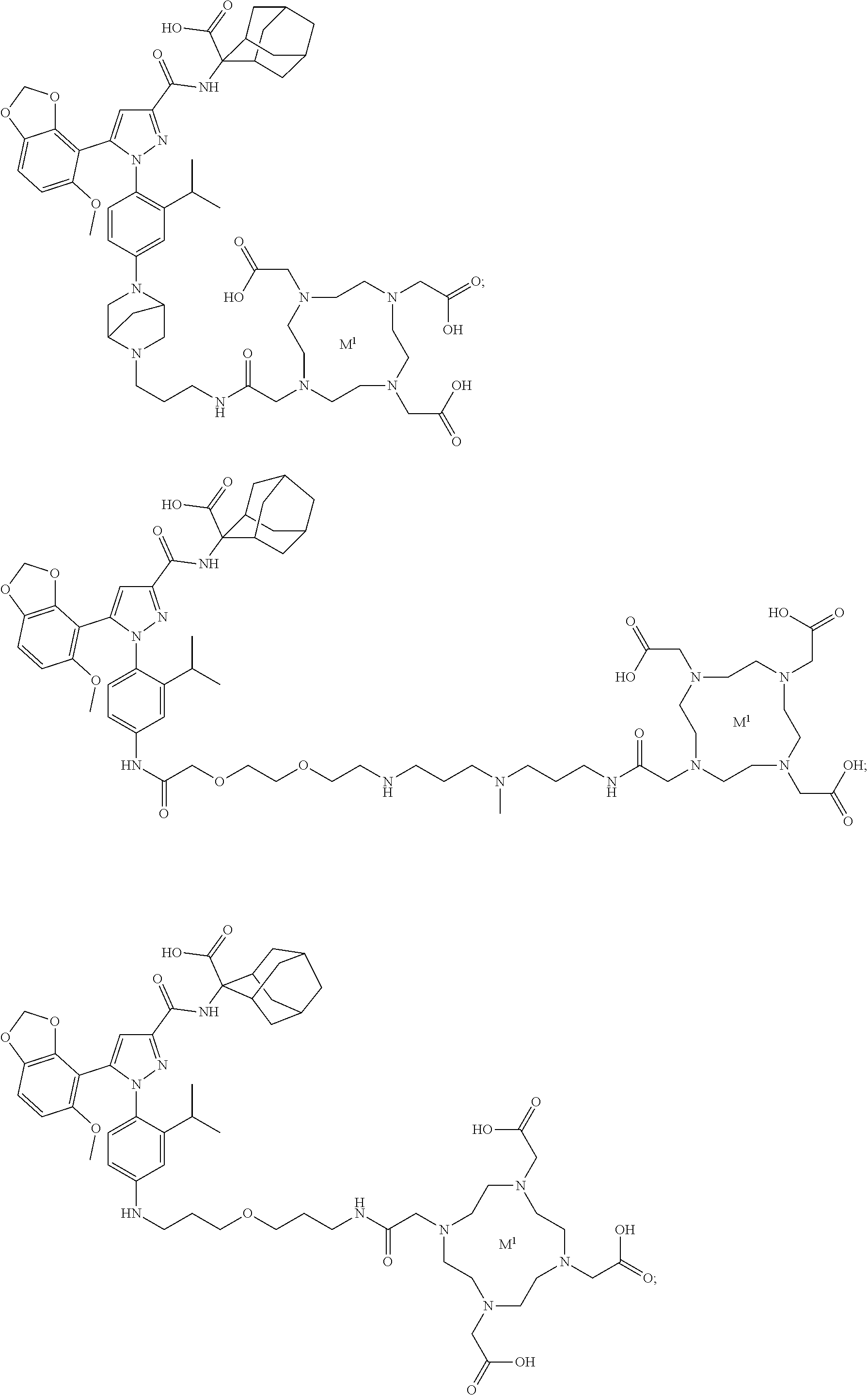

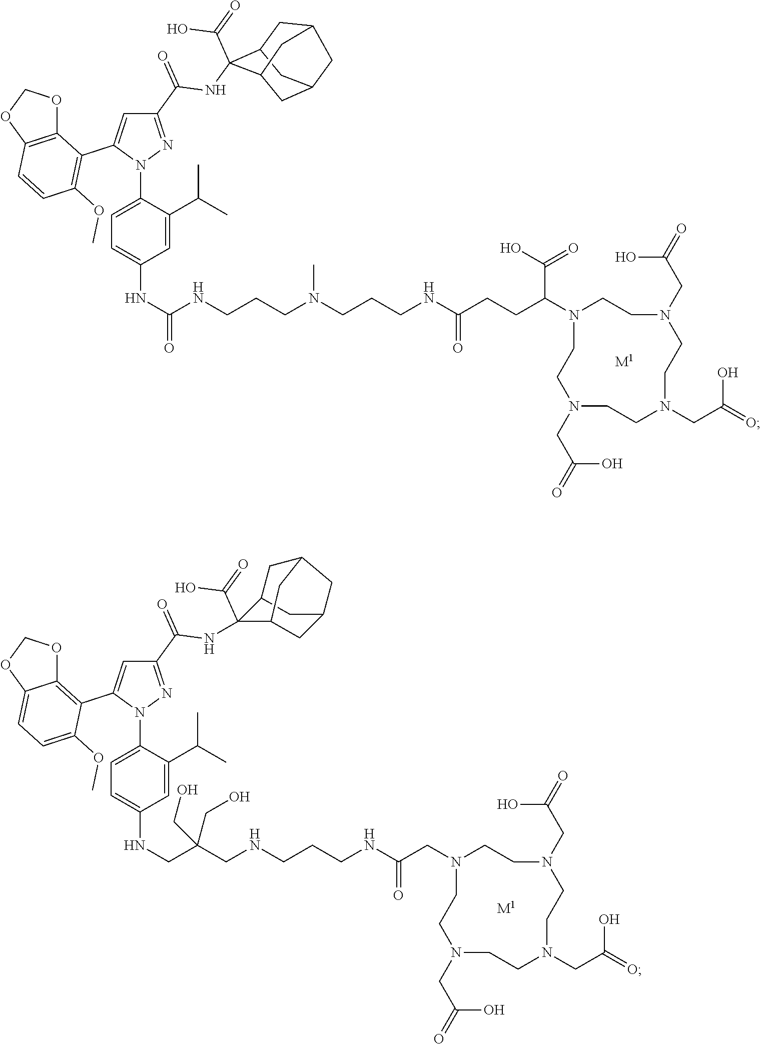

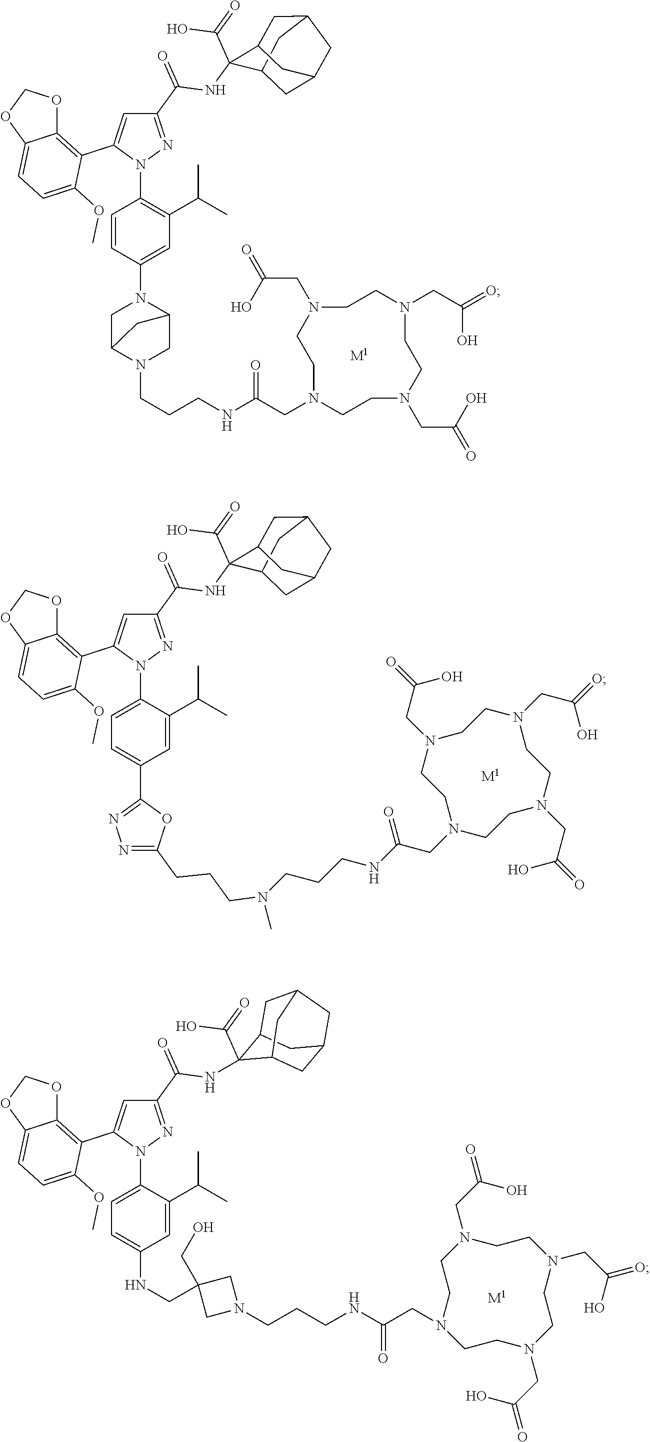

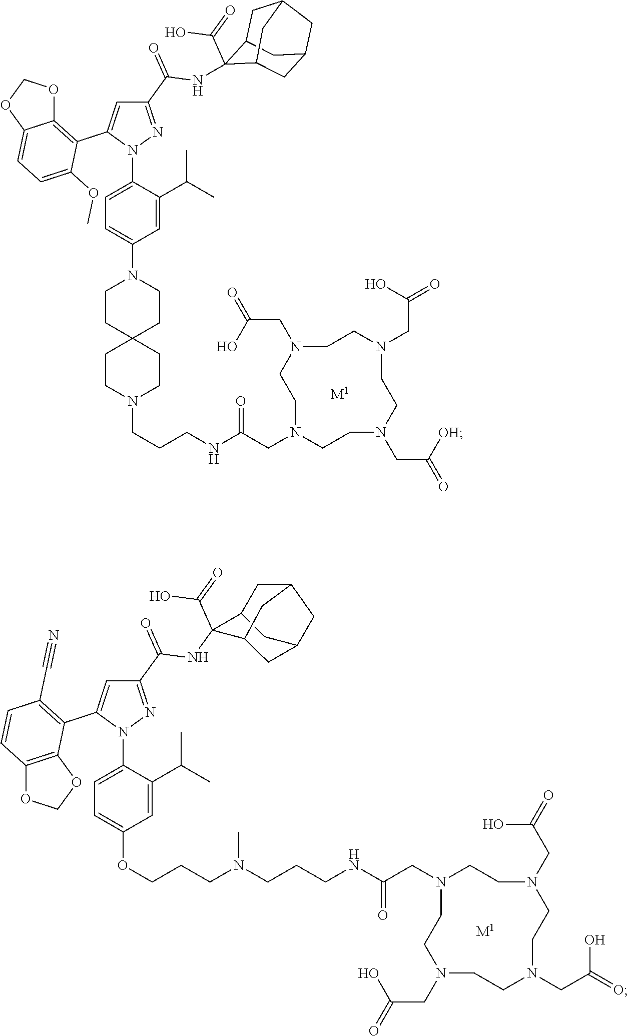

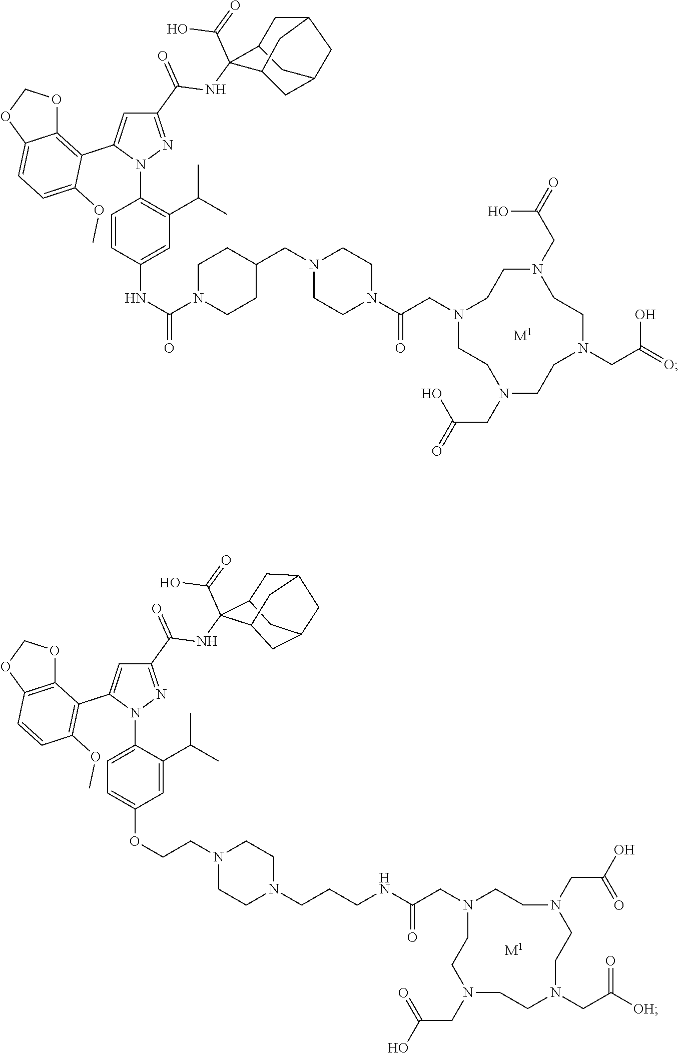

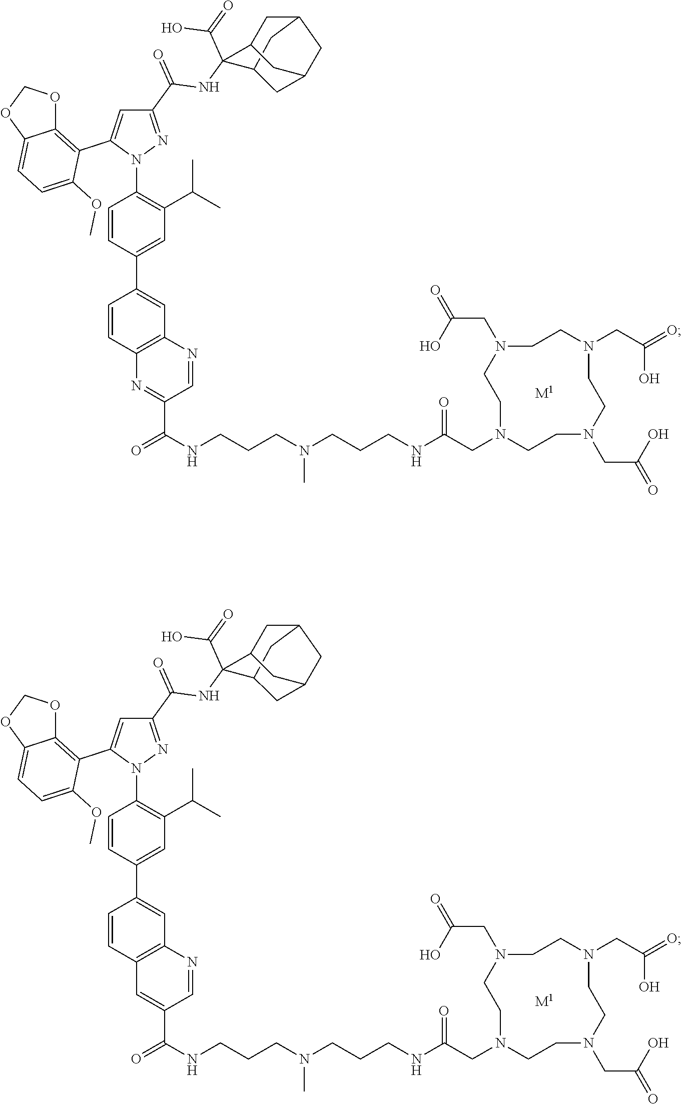

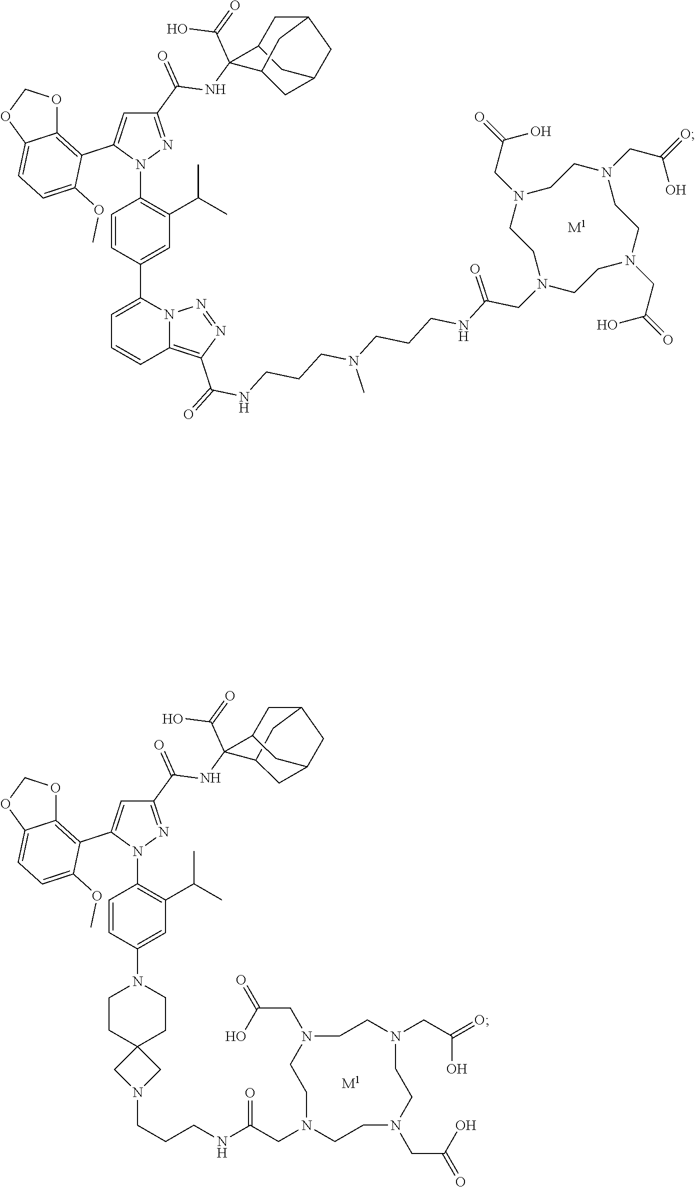

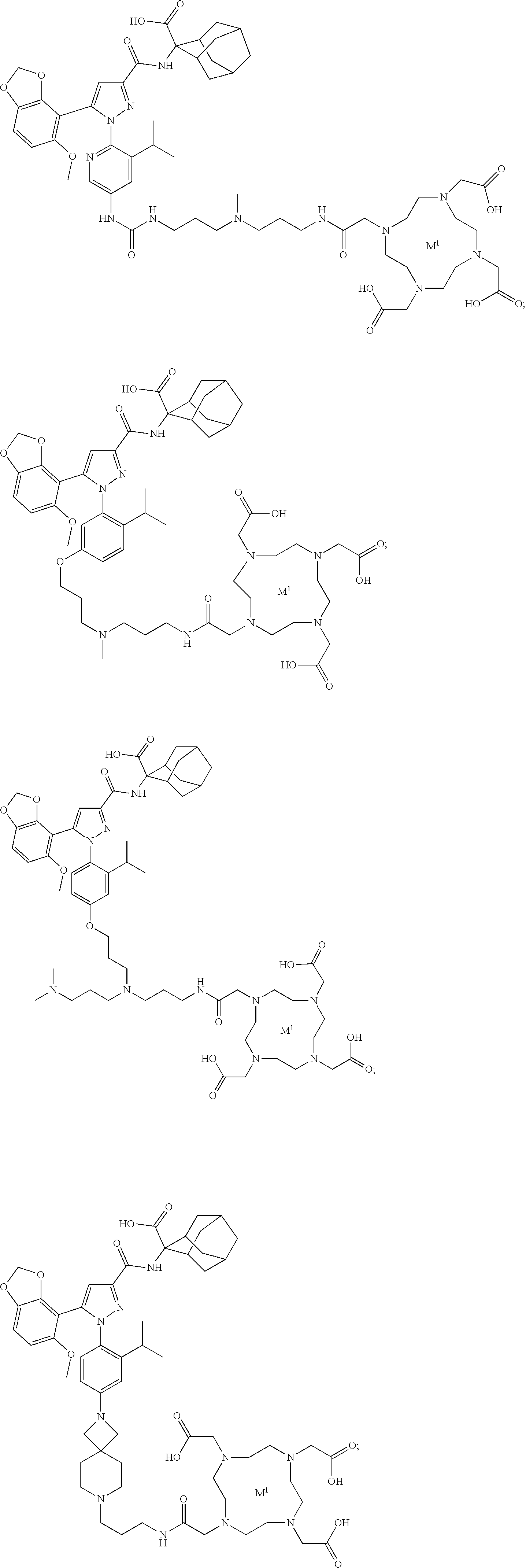

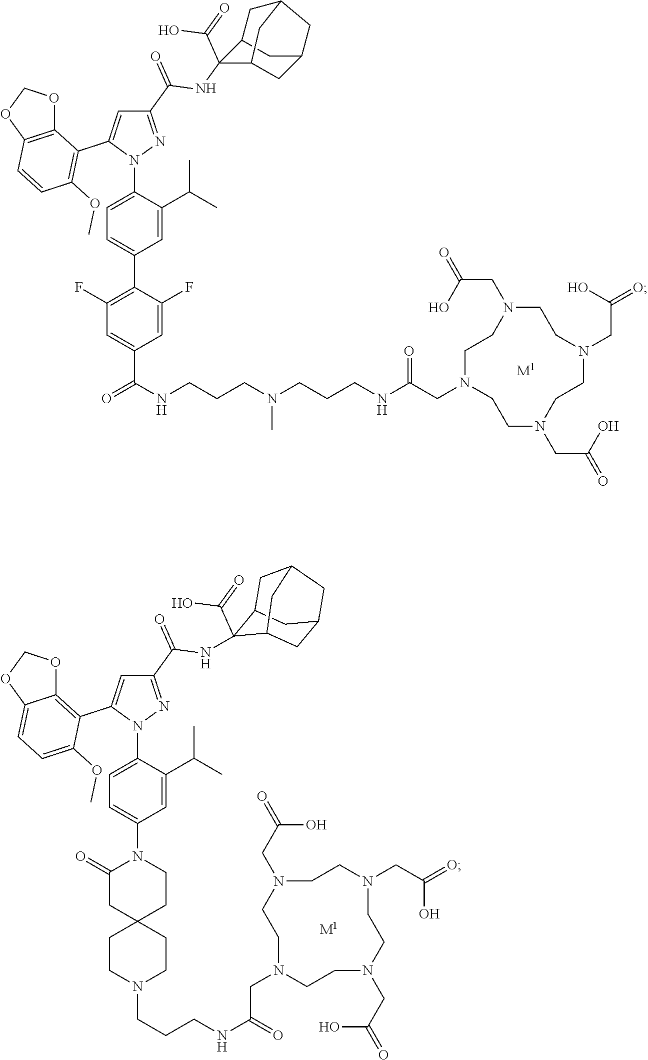

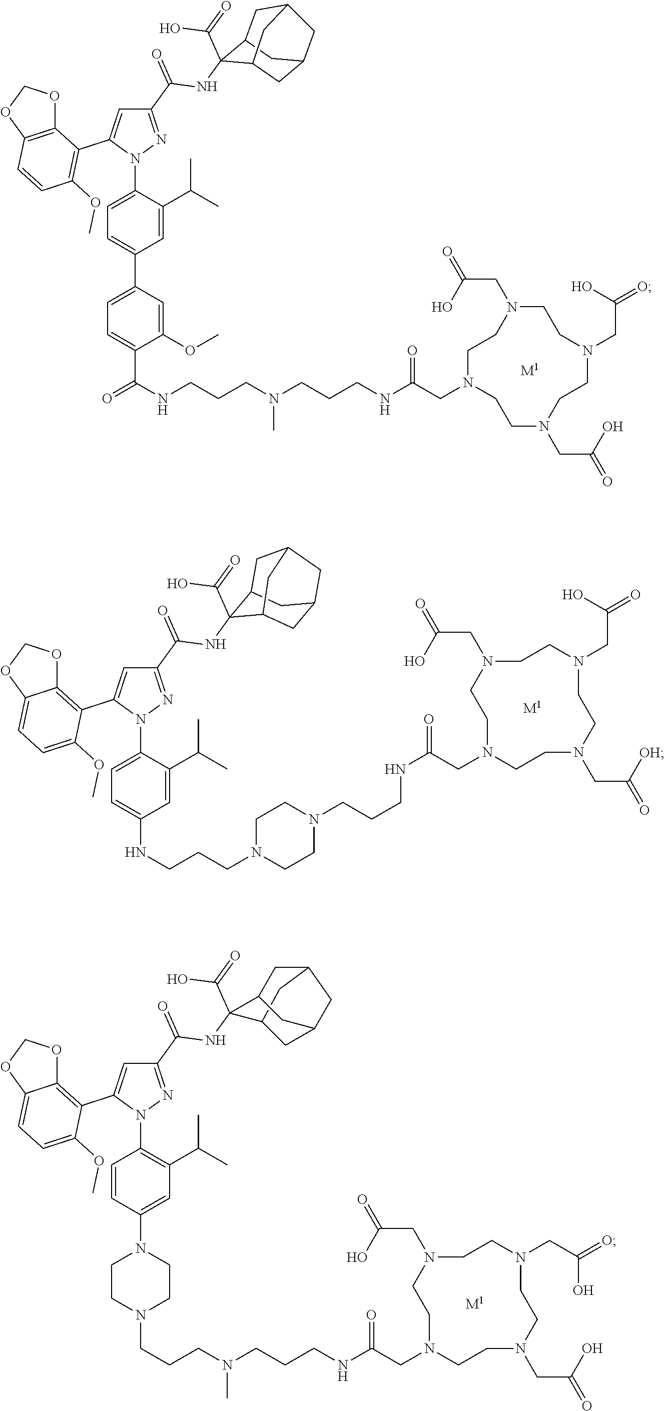

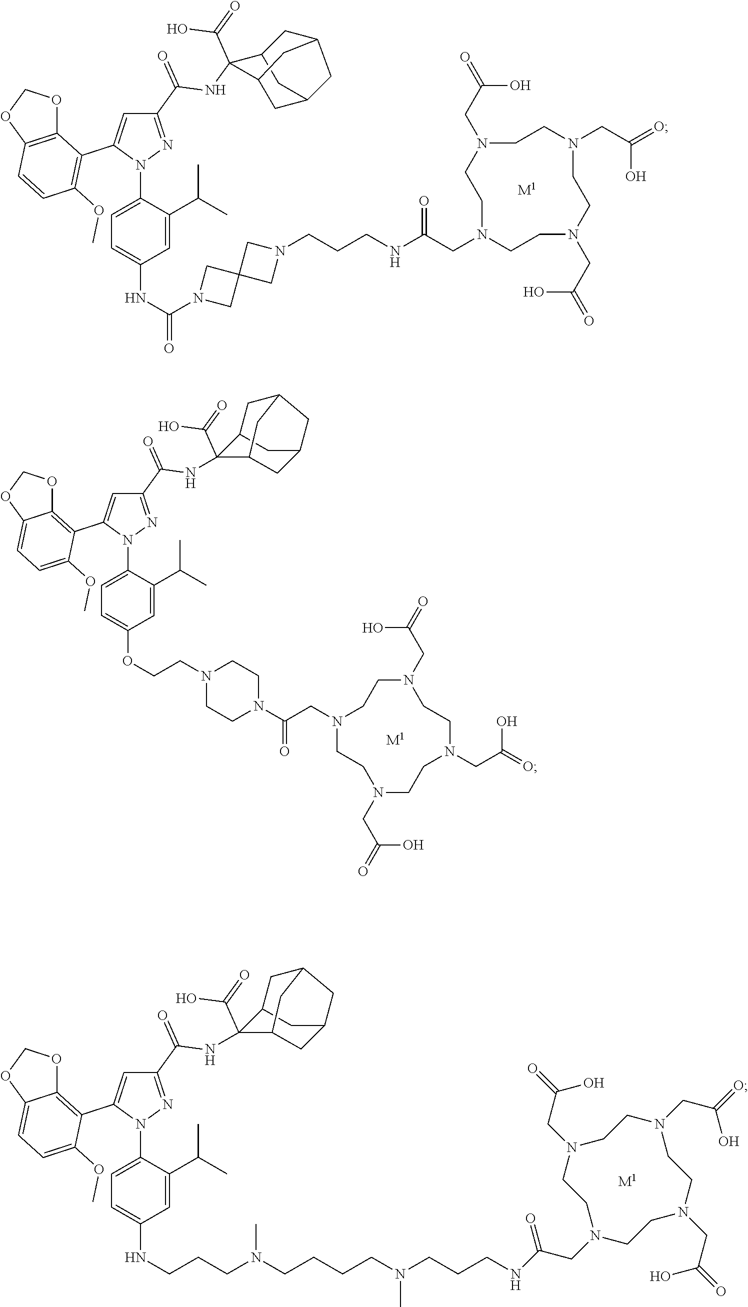

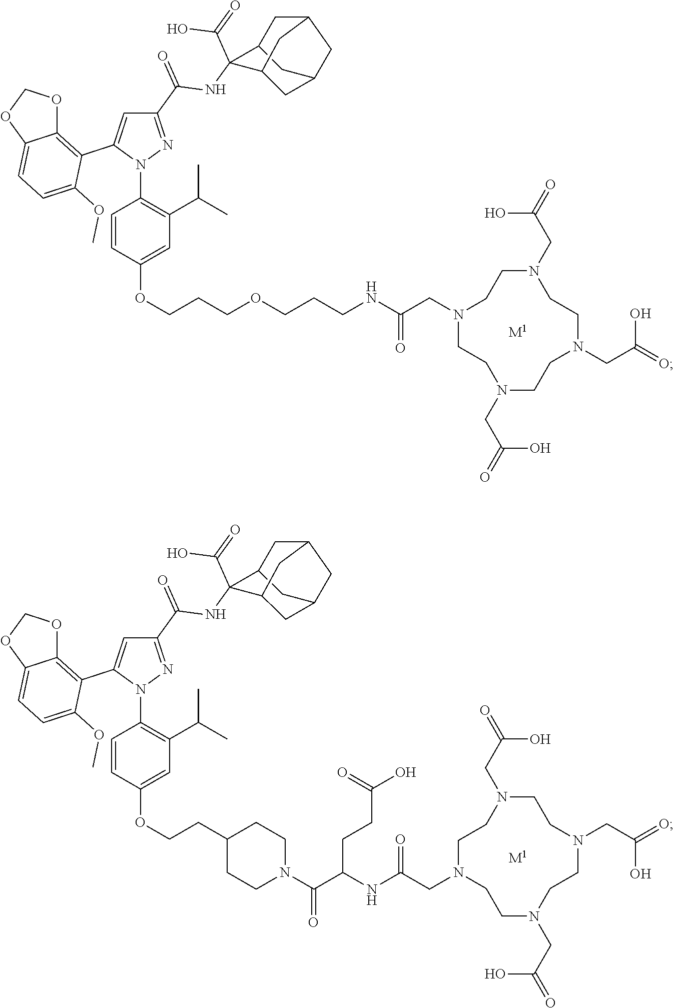

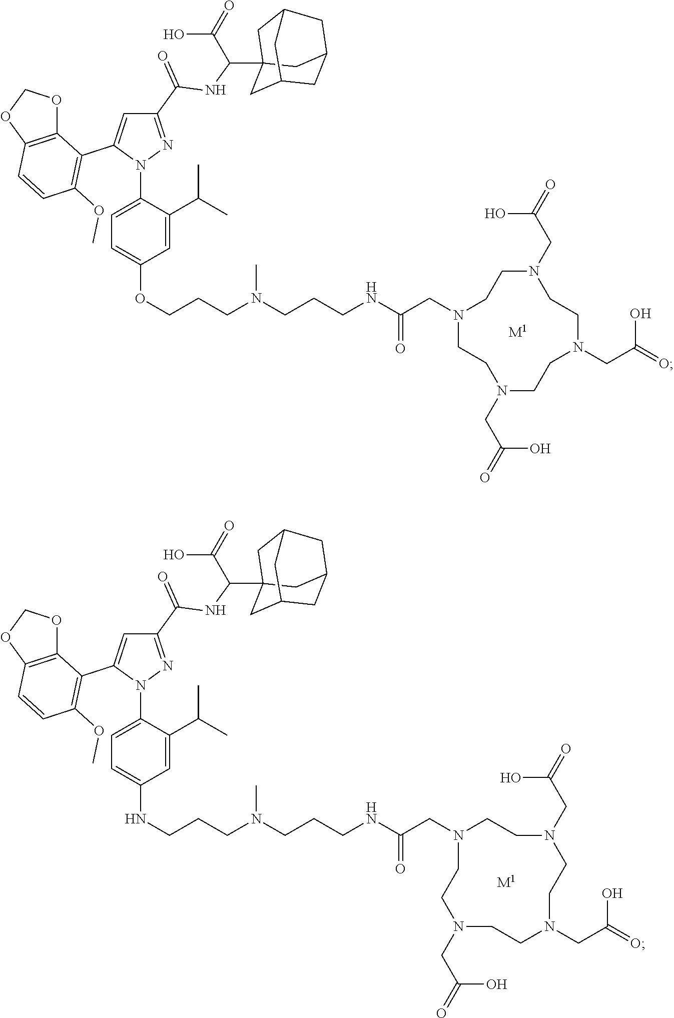

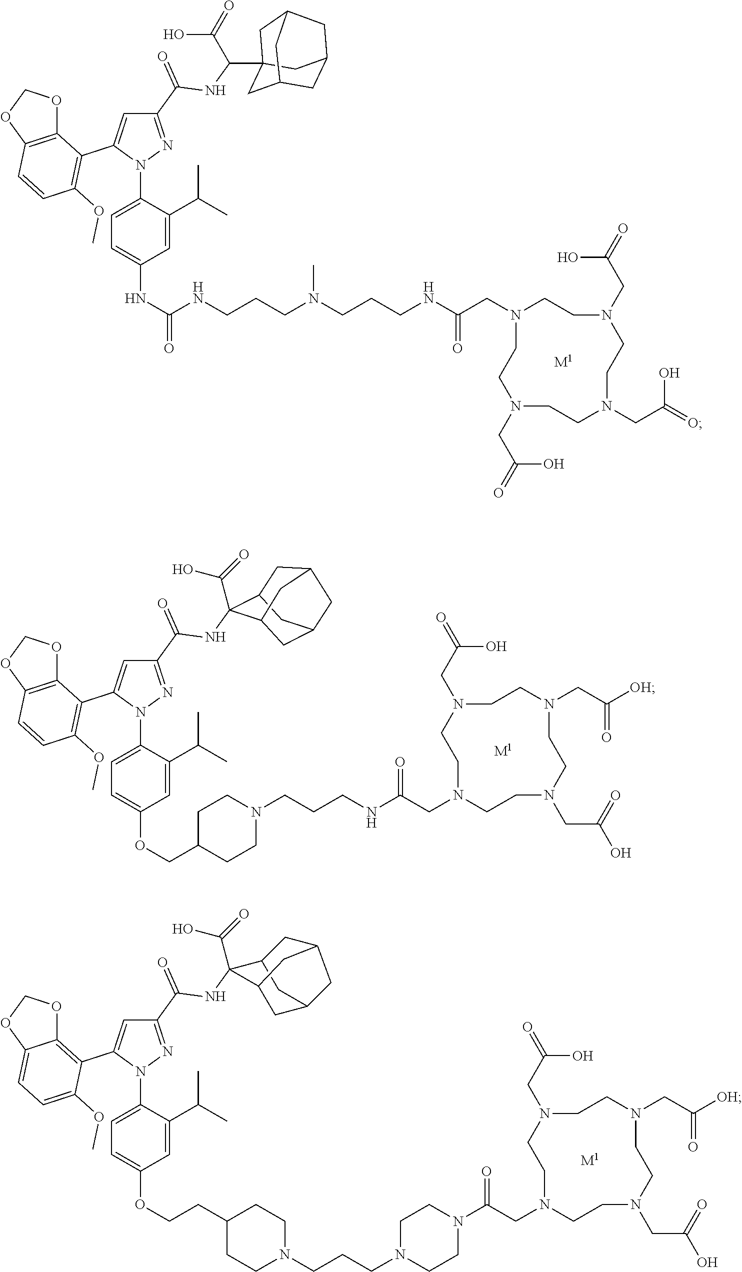

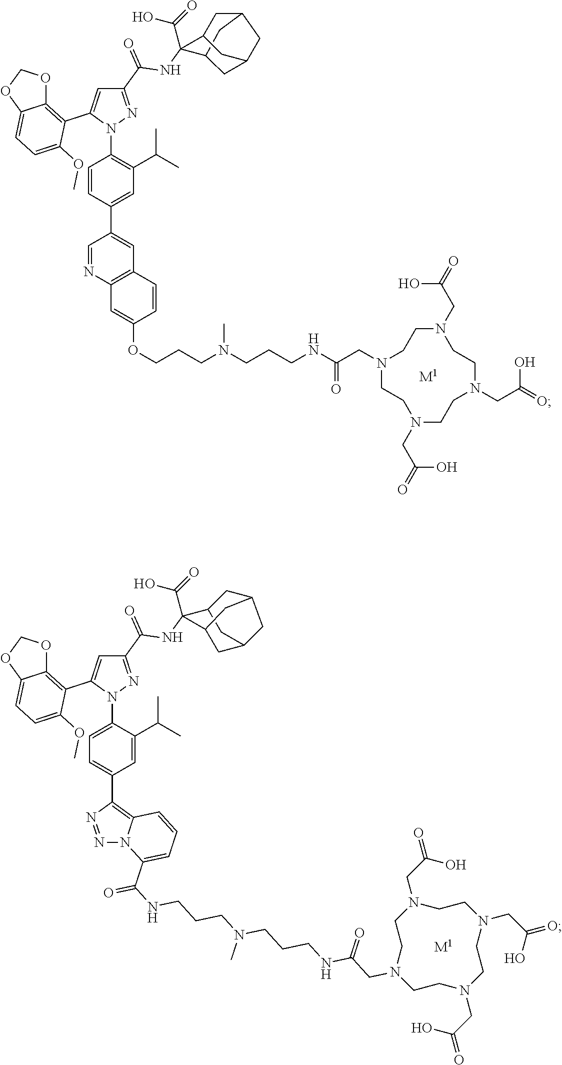

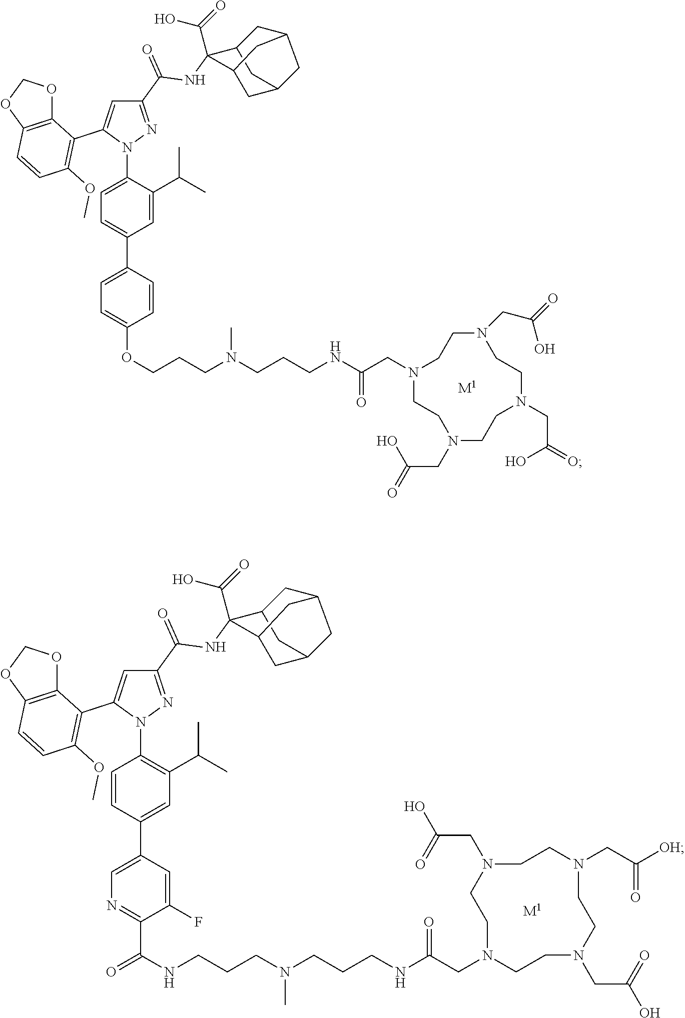

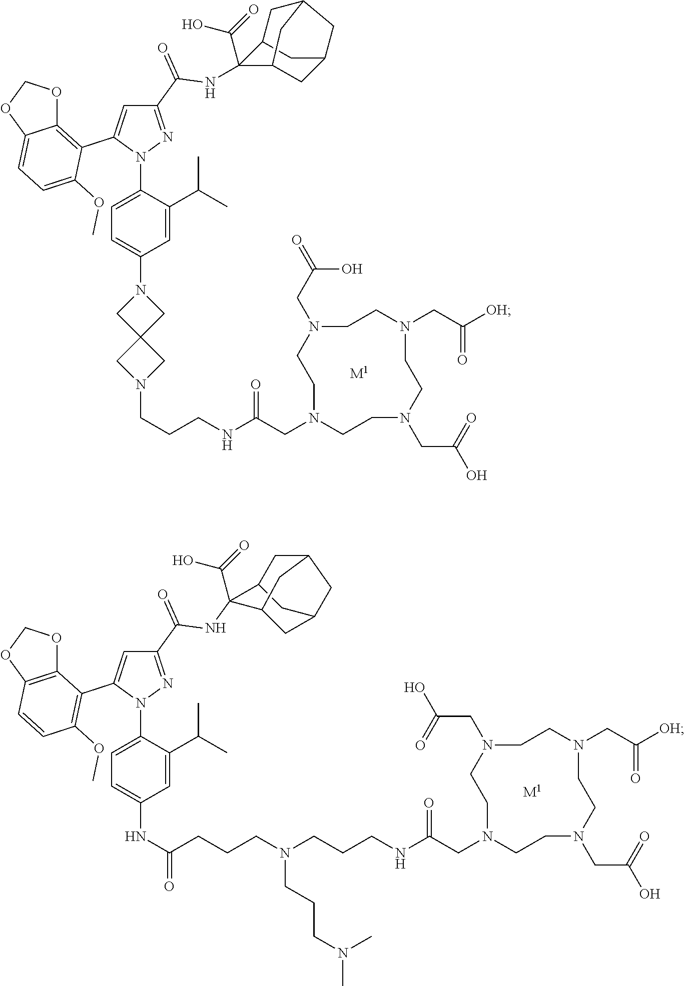

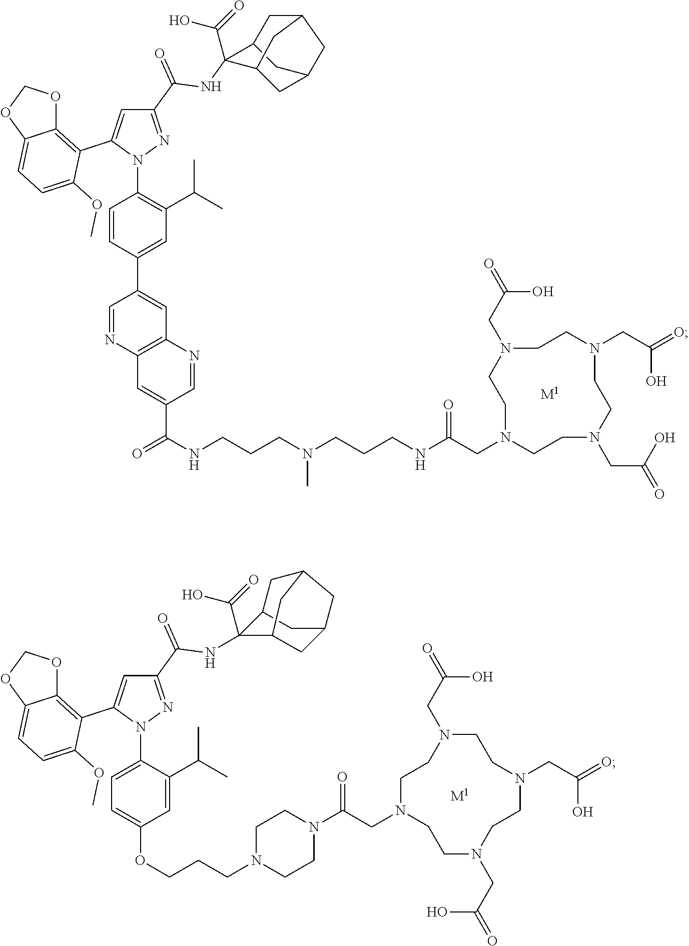

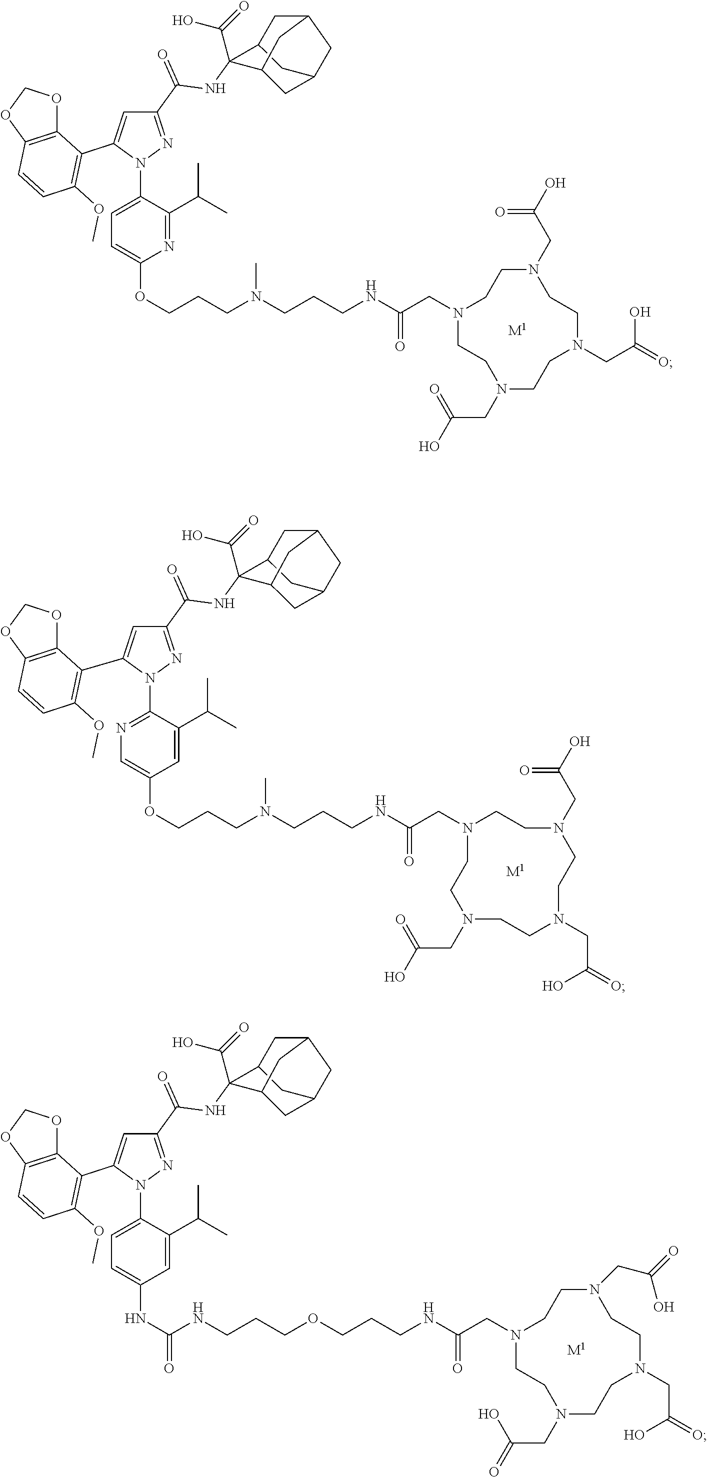

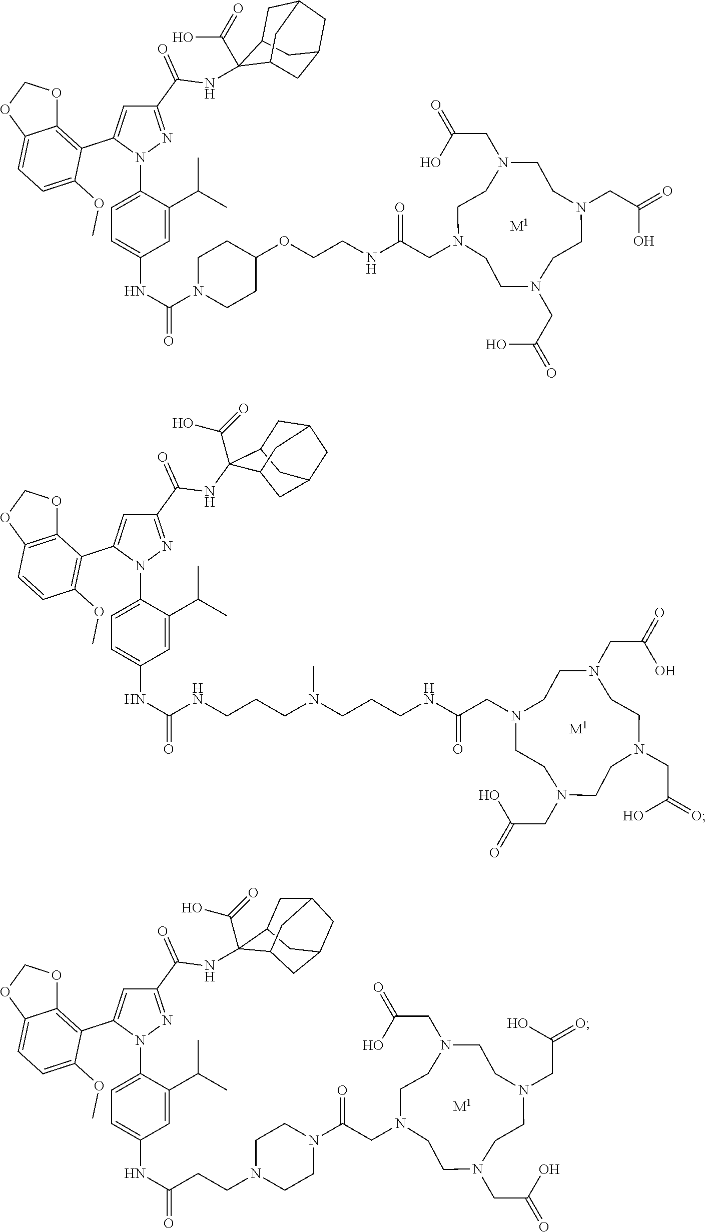

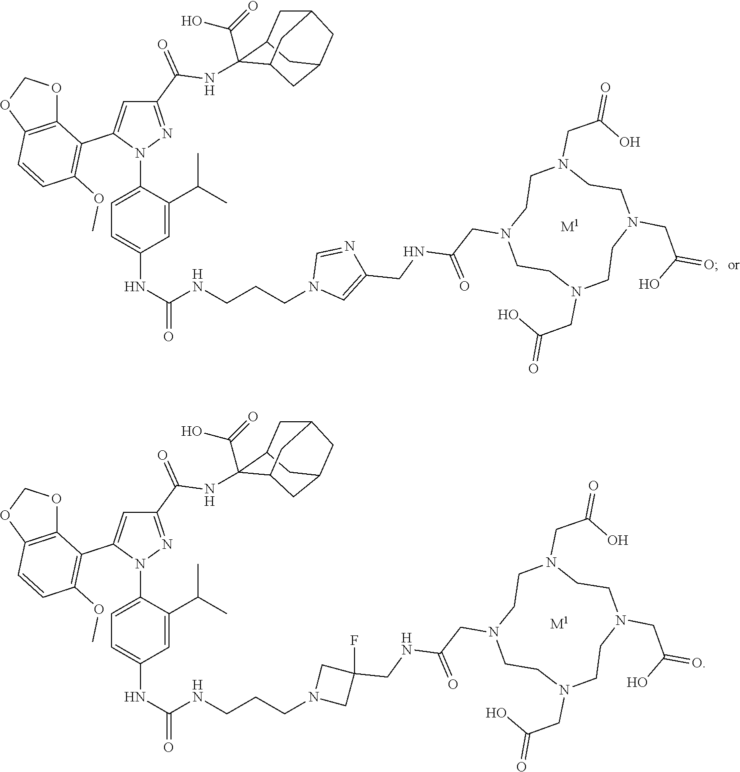

Clause 80. The compound of clause 77-79, or a pharmaceutically acceptable salt thereof, wherein M¹ is ¹⁷⁷Lu, ²¹²Pb, or ²²⁵Ac.

Clause 81. The compound of clause 80, or a pharmaceutically acceptable salt thereof, wherein M¹ is ¹⁷⁷Lu.

Clause 82. A pharmaceutical composition comprising a compound of any one of clauses 1-781, or a pharmaceutically acceptable salt thereof, and a pharmaceutically acceptable carrier, diluent or excipient.

Clause 83. A method of treating cancer, comprising administering to a patient in need thereof, a therapeutically effective amount of a compound of any one of clauses 1-81, or a pharmaceutically acceptable salt thereof.

Clause 84. The method of clause 83, wherein the cancer has one or more cancer cells that overexpress neurotensin receptor 1 (NTSR1).

Clause 85. The method of clause 83 or clause 84, wherein the cancer is breast cancer, colorectal cancer, endometrial cancer, gastric cancer, lung cancer, pancreatic cancer, prostate cancer, head and neck cancer, non-small-cell lung cancer (NSCLC), pleural mesothelioma, head and neck squamous cell carcinoma (HNSCC), glioma, glioblastoma multiforme (GBM), meningioma, Ewing's sarcoma, gastrointestinal stroma tumors, uterine leiomyoma, cutaneous T-cell lymphoma, small cell lung cancer, or pancreatic ductal adenocarcinoma.

Clause 86. A compound of any one of clauses 1-81, or a pharmaceutically acceptable salt thereof for use in therapy.

Clause 87. A compound of any one of clauses 1-81, or a pharmaceutically acceptable salt thereof, for use in the treatment of cancer.

Clause 88. The compound or a pharmaceutically acceptable salt thereof for use of clause 82, wherein the cancer has one or more cancer cells that overexpress neurotensin receptor 1 (NTSR1).

Clause 89. The compound or a pharmaceutically acceptable salt thereof for use of clause 87 or clause 88, wherein the cancer is breast cancer, colorectal cancer, endometrial cancer, gastric cancer, lung cancer, pancreatic cancer, prostate cancer, head and neck cancer, non-small-cell lung cancer (NSCLC), pleural mesothelioma, head and neck squamous cell carcinoma (HNSCC), glioma, glioblastoma multiforme (GBM), meningioma, Ewing's sarcoma, gastrointestinal stroma tumors, uterine leiomyoma, cutaneous T-cell lymphoma, small cell lung cancer, or pancreatic ductal adenocarcinoma.

Clause 90. Use of a compound of any one of claims 1-81, or pharmaceutically acceptable salts thereof, in the manufacture of a medicament for treating cancer.

Clause 91. The use of clause 90, wherein the cancer has one or more cancer cells that overexpress neurotensin receptor 1 (NTSR1).

Clause 92. The use of clause 90 or clause 91, wherein the cancer is breast cancer, colorectal cancer, endometrial cancer, gastric cancer, lung cancer, pancreatic cancer, prostate cancer, head and neck cancer, non-small-cell lung cancer (NSCLC), pleural mesothelioma, head and neck squamous cell carcinoma (HNSCC), glioma, glioblastoma multiforme (GBM), meningioma, Ewing's sarcoma, gastrointestinal stroma tumors, uterine leiomyoma, cutaneous T-cell lymphoma, small cell lung cancer, or pancreatic ductal adenocarcinoma.

Clause 93. The method of any one of clauses 83-85, the compound for use of any one of clauses 87-89, or the use of any one of clauses 85-87, wherein the cancer is breast cancer.

Clause 94. The method of any one of clauses 83-85, the compound for use of any one of clauses 87-89, or the use of any one of clauses 90-92, wherein the cancer is colorectal cancer.

Clause 95. The method of any one of clauses 83-85, the compound for use of any one of clauses 87-89, or the use of any one of clauses 90-92, wherein the cancer is endometrial cancer.

Clause 96. The method of any one of clauses 83-85, the compound for use of any one of clauses 87-89, or the use of any one of clauses 90-92, wherein the cancer is gastric cancer.

Clause 97. The method of any one of clauses 83-85, the compound for use of any one of clauses 87-89, or the use of any one of clauses 90-92, wherein the cancer is lung cancer.

Clause 98. The method of any one of clauses 83-85, the compound for use of any one of clauses 87-89, or the use of any one of clauses 90-92, wherein the cancer is pancreatic cancer.

Clause 99. The method of any one of clauses 83-85, the compound for use of any one of clauses 87-89, or the use of any one of clauses 90-92, wherein the cancer is prostate cancer.

Clause 9100. The method of any one of clauses 83-85, the compound for use of any one of clauses 87-89, or the use of any one of clauses 90-92, wherein the cancer is head and neck cancer.

Clause 101. The method of any one of clauses 83-85, the compound for use of any one of clauses 87-89, or the use of any one of clauses 90-92, wherein the cancer is non-small-cell lung cancer (NSCLC).

Clause 102. The method of any one of clauses 83-85, the compound for use of any one of clauses 87-89, or the use of any one of clauses 90-92, wherein the cancer is pleural mesothelioma.

Clause 103. The method of any one of clauses 83-85, the compound for use of any one of clauses 87-89, or the use of any one of clauses 90-92, wherein the cancer is head and neck squamous cell carcinoma (HNSCC).

Clause 104. The method of any one of clauses 83-85, the compound for use of any one of clauses 87-89, or the use of any one of clauses 90-92, wherein the cancer is glioma.

Clause 105. The method of any one of clauses 83-85, the compound for use of any one of clauses 87-89, or the use of any one of clauses 90-92, wherein the cancer is glioblastoma multiforme (GBM).

Clause 106. The method of any one of clauses 83-85, the compound for use of any one of clauses 87-89, or the use of any one of clauses 90-92, wherein the cancer is meningioma.

Clause 107. The method of any one of clauses 83-85, the compound for use of any one of clauses 87-89, or the use of any one of clauses 90-92, wherein the cancer is Ewing's sarcoma.

Clause 108. The method of any one of clauses 83-85, the compound for use of any one of clauses 87-89, or the use of any one of clauses 90-92, wherein the cancer is gastrointestinal stroma tumors.

Clause 109. The method of any one of clauses 83-85, the compound for use of any one of clauses 87-89, or the use of any one of clauses 90-92, wherein the cancer is uterine leiomyoma.

Clause 110. The method of any one of clauses 83-85, the compound for use of any one of clauses 87-89, or the use of any one of clauses 90-92, wherein the cancer is cutaneous T-cell lymphoma.

Clause 111. The method of any one of clauses 83-85, the compound for use of any one of clauses 87-89, or the use of any one of clauses 90-92, wherein the cancer is small cell lung cancer.

Clause 112. The method of any one of clauses 83-85, the compound for use of any one of clauses 87-89, or the use of any one of clauses 90-92, wherein the cancer is pancreatic ductal adenocarcinoma.

### Definitions

The term “albumin-binding group,” refers to a group capable of binding to albumin by covalent or non-covalent binding. An albumin-binding group may comprise or consist of a group selected from fatty acids, phthalocyanines, coumarins, flavonoids, tetracyclines, naphthalenes, arylcarboxylic acids, heteroarylcarboxylic acids, lipids, alkyl amines, cyclic or linear tetrapyrroles and organometallic compounds thereof, halo-substituted aromatic acid derivatives, organic dyes, and derivatives of tryptophan and thyroxine. Certain exemplary albumin-binding groups include:

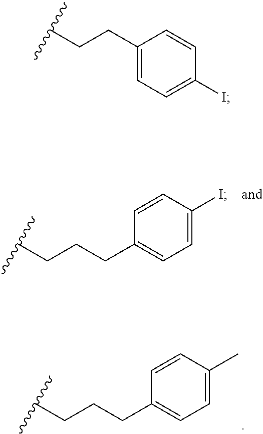 [SMILES: *CCc1ccc(I)cc1 ; *CCCc1ccc(I)cc1 ; *CCCc1ccc(C)cc1]

The term “alkenyl” refers to a straight or branched-chain monovalent hydrocarbon radical containing at least one double bond in the chain. The double bond of an alkenyl group can be unconjugated or conjugated to another unsaturated group. Examples of alkenyl groups include ethenyl, propenyl, n-butenyl, iso-butenyl, pentenyl, or hexenyl.

The term “alkenylene” refers to a straight or branched-chain divalent hydrocarbon radical containing at least one double bond in the chain.

The term “alkyl” refers to a saturated straight or branched-chain monovalent hydrocarbon radical, preferably containing 1-12 carbon atoms, more preferably 1-6 carbon atoms. Examples of a C₁-C₆ alkyl group include, but are not limited to, methyl, ethyl, propyl, butyl, pentyl, hexyl, isopropyl, isobutyl, sec-butyl, tert-butyl, isopentyl, neopentyl, and isohexyl.

The term “alkylene” refers to a saturated straight or branched-chain divalent hydrocarbon radical.

The term “alkynyl” refers to a straight or branched-chain monovalent hydrocarbon radical containing at least one triple bond in the chain. Examples of alkynyl groups include ethynyl, propargyl, n-butynyl, iso-butynyl, pentynyl, or hexynyl.

The term alkynylene” refers to a straight or branched-chain divalent hydrocarbon radical containing at least one triple bond in the chain.

The term “amino” refers to —NH₂.

The term “aryl” unless otherwise specifically defined refers to a cyclic, aromatic hydrocarbon group that has 1 to 3 aromatic rings, including monocyclic, or bicyclic groups such as phenyl, biphenyl, or naphthyl. Where containing two aromatic rings (bicyclic, etc.), the aromatic rings of the aryl group may be joined at a single point (e.g., biphenyl), or fused (e.g., naphthyl). Furthermore, when containing two fused rings the aryl groups herein defined may have one, or more saturated, or partially unsaturated ring fused with a fully unsaturated aromatic ring. Exemplary ring systems of these aryl groups include, but are not limited to, phenyl, biphenyl, naphthyl, anthracenyl, phenalenyl, phenanthrenyl, indanyl, indenyl, tetrahydronaphthalenyl, and tetrahydrobenzoannulenyl.

The term “arylene” refers to a divalent aryl group.

The terms “cancer” and “cancerous” as used herein refer to or describe the physiological condition in patients that is typically characterized by unregulated cell proliferation. Included in this definition are benign and malignant cancers.

The term “chelator,” refers to a group capable of complexing with a radionuclide. A chelator can complex with a radionuclide via one or more functional groups (e.g., carbonyl, carboxylic acid, amino, or amide groups) or atoms (e.g., via nitrogen or oxygen atoms). It is to be understood, that when present, one or more carboxylic acid groups of a chelator may exist as carboxylate anions, and one or more of the carboxylate anions may coordinate to a radionuclide. A chelator may be in completely ionized, partially ionized, or non-ionized form. A chelator complexed with a radionuclide may be illustrated in alternative fashions, for example, a DOTA-based chelator complexed with ¹⁷⁷Lu(3+) may be depicted as below:

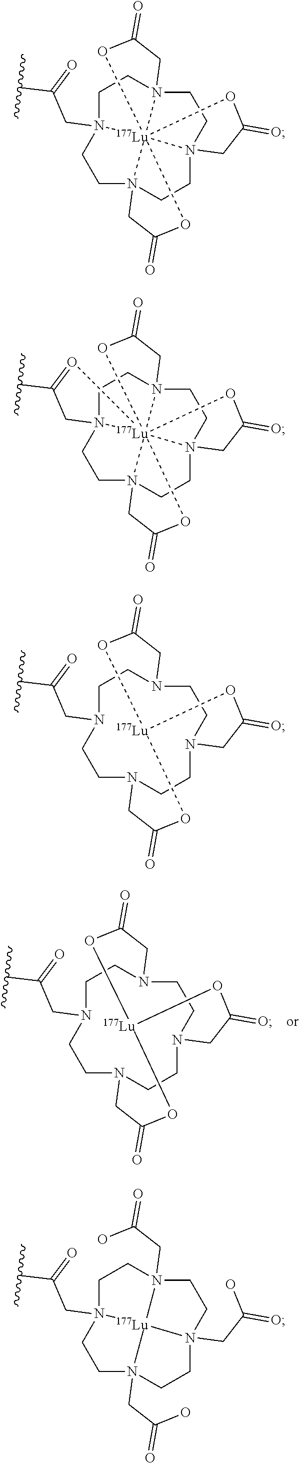

The term “cycloalkyl” unless otherwise specifically defined refers to a mono or polycyclic saturated carbon ring containing 3-18 carbon atoms, preferably 3-10 carbon atoms. Examples of cycloalkyl groups include, without limitation, cyclopropyl, cyclobutyl, cyclopentyl, cyclohexyl, cycloheptanyl, cyclooctanyl, norbornyl, bicyclo[2.2.2]octanyl, and adamantyl.

The term “cycloalkylene” refers to a divalent cycloalkyl group.

The term “diagnostically effective amount” when used in connection with a compound refers to the amount or dose of the compound which upon single or multiple dose administration to the patient provides the desired diagnostic effect. A diagnostically effective amount can be determined by one skilled in the art by use of known techniques.

The term “halogen” or “halo” refers to fluorine, chlorine, bromine, or iodine.

The term “heteroaryl” unless otherwise specifically defined refers to a monovalent monocyclic or polycyclic aromatic radical of 5 to 24 ring atoms, preferably 5 to 10 ring atoms, containing one or more ring heteroatoms selected from N, O, S, P, or B, preferably 1, 2, 3, or 4 ring heteroatoms selected from N, O, or S, the remaining ring atoms being carbon atoms. Examples of heteroaromatic groups include, but are not limited to, furyl, thienyl, pyrrolyl, pyridyl, pyrazolyl, pyrimidinyl, imidazolyl, isoxazolyl, oxazolyl, oxadiazolyl, pyrazinyl, indolyl, thiophen-2-yl, quinolyl, benzopyranyl, isothiazolyl, thiazolyl, thiadiazole, indazole, benzimidazolyl, thieno[3,2-b]thiophene, triazolyl, triazinyl, imidazo[1,2-b]pyrazolyl, furo[2,3-c]pyridinyl, imidazo[1,2-a]pyridinyl, indazolyl, pyrrolo[2,3-c]pyridinyl, pyrrolo[3,2-c]pyridinyl, pyrazolo[3,4-c]pyridinyl, thieno[3,2-c]pyridinyl, thieno[2,3-c]pyridinyl, thieno[2,3-b]pyridinyl, benzothiazolyl, indolyl, indolinyl, indolinonyl, dihydrobenzothiophenyl, dihydrobenzofuranyl, benzofuranyl, chromanyl, thiochromanyl, tetrahydroquinolinyl, dihydrobenzothiazinyl, quinolinyl, isoquinolinyl, 1,6-naphthyridinyl, benzo[de]isoquinolinyl, pyrido[4,3-b][1,6]naphthyridinyl, thieno[2,3-b]pyrazinyl, quinazolinyl, tetrazolo[1,5-a]pyridinyl, [1,2,4]triazolo[4,3-a]pyridinyl, isoindolyl, pyrrolo[2,3-b]pyridinyl, pyrrolo[3,4-b]pyridinyl, pyrrolo[3,2-b]pyridinyl, imidazo[5,4-b]pyridinyl, pyrrolo[1,2-a]pyrimidinyl, tetrahydro pyrrolo[1,2-a]pyrimidinyl, 3,4-dihydro-2H-1-pyrrolo[2,1-b]pyrimidine, dibenzo[b,d]thiophene, pyridin-2-one, furo[3,2-c]pyridinyl, furo[2,3-c]pyridinyl, 1H-pyrido[3,4-b][1,4]thiazinyl, benzooxazolyl, benzoisoxazolyl, furo[2,3-b]pyridinyl, benzothiophenyl, 1,5-naphthyridinyl, furo[3,2-b]pyridine, [1,2,4]triazolo[1,5-a]pyridinyl, benzo[1,2,3]triazolyl, imidazo[1,2-a]pyrimidinyl, [1,2,4]triazolo[4,3-b]pyridazinyl, benzo[c][1,2,5]thiadiazolyl, benzo[c][1,2,5]oxadiazole, 1,3-dihydro-2H-benzo[d]imidazol-2-one, 3,4-dihydro-2H-pyrazolo[1,5-b][1,2]oxazinyl, 4,5,6,7-tetrahydropyrazolo[1,5-a]pyridinyl, thiazolo[5,4-d]thiazolyl, imidazo[2,1-b][1,3,4]thiadiazolyl, thieno[2,3-b]pyrrolyl, and 3H-indolyl. Furthermore, when containing two, or more fused rings, the heteroaryl groups defined herein may have one, or more saturated, or partially unsaturated ring fused with one, or more fully unsaturated aromatic ring. In heteroaryl ring systems containing more than two fused rings, a saturated, or partially unsaturated ring may further be fused with a saturated, or partially unsaturated ring described herein. Furthermore, when containing three, or more fused rings, the heteroaryl groups defined herein may have one, or more saturated, or partially unsaturated ring spiro-fused. A saturated or partially unsaturated ring described herein may be optionally substituted with one or more oxo. Exemplary ring systems of these heteroaryl groups include, for example, indolinyl, indolinonyl, dihydrobenzothiophenyl, dihydrobenzofuran, chromanyl, thiochromanyl, tetrahydroquinolinyl, dihydrobenzothiazine, 3,4-dihydro-1H-isoquinolinyl, 2,3-dihydrobenzofuranyl, benzofuranonyl, oxindolyl, indolyl, 1,6-dihydro-7H-pyrazolo[3,4-c]pyridin-7-onyl, 7,8-dihydro-6H-pyrido[3,2-b]pyrrolizinyl, 8H-pyrido[3,2-b]pyrrolizinyl, 1,5,6,7-tetrahydrocyclopenta[b]pyrazolo[4,3-e]pyridinyl, 7,8-dihydro-6H-pyrido[3,2-b]pyrrolizinyl, pyrazolo[1,5-a]pyrimidin-7 (4H)-onyl, 3,4-dihydropyrazino[1,2-a]indol-1 (2H)-onyl, benzo[c][1,2]oxaborol-1 (3H)-olyl, 6,6a, 7,8-tetrahydro-9H-pyrido[2,3-b]pyrrolo[1,2-d][1,4]oxazin-9-onyl, and 6a′,7′-dihydro-6′H,9′H-spiro[cyclopropane-1,8′-pyrido[2,3-b]pyrrolo[1,2-d][1,4]oxazin]-9′-onyl.

The term “heteroarylene” refers to a divalent heteroaryl group.

The term “heterocyclyl”, “heterocycle”, or “heterocycloalkyl” unless otherwise specifically defined refers to a mono or polycyclic ring containing 3-24 atoms, preferably 3-10 atoms, which include carbon, and one or more heteroatoms selected from N, O, S, P, or B, preferably 1, 2, 3, or 4 heteroatoms selected from N, O, and S. As used herein, “heterocycle” may be aromatic or non-aromatic. The term therefore encompasses heteroaryl rings, which are aromatic heterocycles and are typically 5- to 10-membered monocyclic or fused bicyclic ring systems as defined for “heteroaryl,” as well as saturated or partially unsaturated heterocyclic rings, which are sometimes referred to as heterocycloalkyl or heterocycloalkenyl and generally comprise 4- to 10-membered rings. Any of these heterocycles may be fused, bridged, or spirocyclic as defined herein. Unless otherwise specified, a heterocycle is optionally substituted with 1-3 substituents independently selected from halo, trifluoromethyl, trifluoromethoxy, hydroxy, cyano, nitro, C₁-C₆ alkyl, C₁-C₆ alkoxy, C₁-C₆ alkylamino, C₁-C₆ haloalkoxy, or C₁-C₆ dialkylamino. Examples of heterocyclyl rings include, but are not limited to, oxetanyl, azetidinyl, tetrahydrofuranyl, tetrahydropyranyl, pyrrolidinyl, oxazolinyl, oxazolidinyl, thiazolinyl, thiazolidinyl, pyranyl, thiopyranyl, tetrahydropyranyl, dioxalinyl, piperidinyl, morpholinyl, thiomorpholinyl, thiomorpholinyl S-oxide, thiomorpholinyl S-dioxide, piperazinyl, azepinyl, oxepinyl, diazepinyl, tropanyl, oxazolidinonyl, and homotropanyl. Unless otherwise indicated, “heterocyclyl” includes rings that are saturated or partially unsaturated and may bear one or more ring “oxo” substituents. For example, carbonyl groups embedded in the ring, forming for example lactam, lactone, imide, cyclic urea, cyclic carbamate, or cyclic carbonate functionalities, provided the ring system is non-aromatic. Examples include, but are not limited to pyrrolidin-2-one-yl(2-oxopyrrolidinyl), piperidin-2-one-yl, morpholin-3-one-yl, 1,3-oxazinan-2-one-yl, imidazolidinone-yl, oxazolidinone-yl, thiazolidinone-yl, succinimidyl, and 1,3-dioxolan-2-one-yl.

The term “heterocycloalkenyl” refers to a non-aromatic mono- or polycyclic monovalent radical containing 3-24 ring atoms (preferably 3-10) in which one or more ring atoms are heteroatoms selected from N, O, S, P, or B (preferably N, O, or S), and in which the ring system contains at least one endocyclic carbon-carbon double bond. Unless otherwise indicated, heterocycloalkenyl groups may be fused, bridged, or spirocyclic as defined herein; may be attached via a ring carbon or ring heteroatom; and may include oxidized heteroatoms (e.g., N-oxides, S-oxides, S,S-dioxides) and/or ring “oxo” substituents (e.g., lactams). Non-limiting examples include 2,3-dihydrofuranyl, 3,4-dihydropyranyl, 2,3-dihydro-1,4-dioxinyl, 1,2-dihydropyridinyl, 3,4-dihydropyrazinyl, and 2,3-dihydro-1,3-oxazinyl.

The term “heterocycloalkylene” refers to a divalent non-aromatic heterocyclyl group. Unless otherwise indicated, attachment may occur via a ring carbon or a ring heteroatom, and the ring may be fused, bridged, or spirocyclic and may include oxidized heteroatoms or ring “oxo” substituents.

The term “fused” (or “fused ring system”) refers to two rings that share two adjacent atoms and the bond between them (a common edge). The fused rings may be carbocyclic or heterocyclic and may be aromatic, partially unsaturated, or saturated. The descriptor may modify aryl, heteroaryl, cycloalkyl, heterocycloalkyl, or heterocycloalkenyl groups.

The term “bridged” (or “bridged bicyclic/tricyclic ring system”) refers to a polycyclic system having two bridgehead atoms connected by one or more bridges such that the rings share the bridgeheads but do not share a single common edge. Such systems are commonly described by von Baeyer notation (e.g., bicyclo[a.b.c]). Non limiting examples include norbornyl (bicyclo[2.2.1]heptanyl), bicyclo[2.2.2]octanyl, quinuclidinyl, and adamantyl (including heteroatom containing analogs). The descriptor may modify aryl, heteroaryl, cycloalkyl, heterocycloalkyl, or heterocycloalkenyl groups.

The term “spirocyclic” (or “spiro”) refers to a polycyclic system in which two rings share exactly one common atom (the spiro atom), which may be carbon or a heteroatom; the rings otherwise do not share atoms or an edge. The descriptor may modify aryl, heteroaryl, cycloalkyl, heterocycloalkyl, or heterocycloalkenyl groups.

Ring size conventions for fused/bridged/spiro systems. Unless otherwise specified, when a ring size is recited for a fused, bridged, spiro, or other polycyclic system, the descriptor refers to the ring that includes the point of attachment to the remainder of the molecule. In spiro systems, the spiro junction atom is counted in the size of each ring. In bridged systems, ring sizes are assigned using the smallest set of unique rings in accordance with standard nomenclature conventions, including von Baeyer notation.

The term “imaging effective amount” when used in connection with a compound refers to the amount or dose of the compound which upon single or multiple dose administration to the patient provides the desired imaging effect. An imaging effective amount can be determined by one skilled in the art by use of known techniques. Known imaging techniques include positron emission tomography, single photon emission computerized tomography, and radioisotope renography.

The term “isomers” refers to compounds that have the same molecular formula but differ in the nature or sequence of bonding of their atoms or the arrangement of their atoms in space. Isomers that differ in the arrangement of their atoms in space are termed “stereoisomers”. Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomers or as a mixture thereof. A mixture containing equal proportions of the enantiomers is called a “racemic mixture”.

The term “linker,” refers to a molecular structure that connects two or more other molecular structures together. In exemplary embodiments, a linker includes one or more groups (e.g., 1-20 groups, 1-10 groups, 1-5 groups, or 1-3 groups) independently selected from amine, ether, thioether, carbonyl, thiocarbonyl, sulfone, sulfoxide, urea, thiourea, and amide; and one or more groups (e.g., 1-20 groups, 1-10 groups, 1-5 groups, or 1-3 groups) independently selected from C₁-C₂₀ alkylene, C₂-C₂₀ alkenylene, and C₂-C₂₀ alkynylene, any of which are optionally interrupted by one or more groups (e.g., 1-20 groups, 1-10 groups, 1-5 groups, or 1-3 groups) independently selected from amine, ether, thioether, carbonyl, thiocarbonyl, sulfone, sulfoxide, urea, thiourea, amide, C₃-C₁₀ cycloalkylene, C₄-C₁₀ heterocycloalkylene, C₆-C₁₀ arylene, and C₅-C₁₀ heteroarylene; wherein the one or more C₁-C₂₀ alkylene, C₂-C₂₀ alkenylene, C₂-C₂₀ alkynylene, C₃-C₁₀ cycloalkylene, C₄-C₁₀ heterocycloalkylene, C₆-C₁₀ arylene, and C₅-C₁₀ heteroarylene at each occurrence are optionally substituted with one or more groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl at each occurrence are optionally substituted with one or more groups independently selected from halo, —CO₂H, —OH, —NH₂, —NHC₁-C₄ alkyl, and —N(C₁-C₄ alkyl)(C₁-C₄ alkyl). In certain embodiments, a linker includes one or more albumin-binding groups. In certain embodiments, a linker includes one albumin-binding group. In certain embodiments, a linker includes zero albumin-binding groups.

The term “5-membered heteroaryl” unless otherwise specifically defined refers to a monovalent monocyclic aromatic radical of 5 ring atoms, containing one or more ring heteroatoms selected from N, O, S, P, or B, preferably 1, 2, 3, or 4 ring heteroatoms selected from N, O, or S, the remaining ring atoms being carbon atoms. Exemplary 5-membered heteroaryl groups include, but are not limited to, furyl, thiophenyl, pyrrolyl, pyrazolyl, imidazolyl, isoxazolyl, oxazolyl, oxadiazolyl, isothiazolyl, thiazolyl, thiadiazole, triazolyl and tetrazolyl.

The term “6-membered heteroaryl” unless otherwise specifically defined refers to a monovalent monocyclic aromatic radical of 6 ring atoms, containing one or more ring heteroatoms selected from N, O, S, P, or B, preferably 1, 2, 3, or 4 ring heteroatoms selected from N, O, or S, the remaining ring atoms being carbon atoms. Exemplary 6-membered heteroaryl groups include, but are not limited to, pyridyl, pyridazinyl, pyrimidinyl, pyrazinyl, or triazinyl.

The term “optionally substituted” refers to a group that is unsubstituted or substituted with one or more of the referenced substituent(s).

The term “patient”, or “subject” is a mammal, e.g., a human, mouse, rat, guinea pig, dog, cat, horse, cow, pig, or non-human primate, such as a monkey, chimpanzee, baboon, or rhesus. Preferably, the mammal is human.

The term “therapeutically effective amount” when used in connection with a compound refers to the amount or dose of the compound which upon single or multiple dose administration to the patient provides the desired effect in the patient under treatment. A therapeutically effective amount can be determined by one skilled in the art by use of known techniques. In determining the therapeutically effective amount for a patient, a number of factors may be considered, including, but not limited to: the species of patient; its size, age, and general health; the specific disease or disorder involved; the degree of or involvement or the severity of the disease or disorder; the response of the individual patient; the particular compound administered; the mode of administration; the bioavailability characteristics of the preparation administered; the dose regimen selected; the use of concomitant medication; and other relevant circumstances.

The term “treating” with regard to a subject, includes restraining, slowing, stopping, or reversing the progression or severity of an existing symptom or disorder.

### EXAMPLES

Certain abbreviations are defined as follows: “AcOH” refers to acetic acid; “ACN” refers to acetonitrile; “aq” refers to aqueous; “B2pin2” refers to Bis(pinacolato)diboron; “Boc₂O” refers to Di-tert-butyl decarbonate; “Burgess reagent” refers to methyl N-[(triethylazaniumyl)sulfonyl]carbamate oxidanide; “BuLi” refers to butyllithium; “CDMT” refers to 2-Chloro-4,6-dimethoxy-1,3,5-triazine; “CO” refers to carbon monoxide; “Cs₂CO₃” refers to cesium carbonate; “CuI” refers to copper iodide; “CuSO₄” refers to copper sulfate; “DBU” refers to 1,8-Diazabicyclo[5.4.0]undec-7-ene; “DCC” refers to N,N′-dicyclohexylcarbodiimide; “DCM” refers to dichloromethane; “DIAD” refers to diisopropyl azodicarboxylate; “DIEA” refers to N,N-diisopropylethylamine; “DMAP” refers to 4-dimethylaminopyridine; “DMF” refers to dimethylformamide; “DMP” refers to Dess-Martin periodinane; “DMSO” refers to dimethyl sulfoxide; “EA” refers to ethyl acetate; “EDCI” refers to 1-Ethyl-3-(3-dimethylaminopropyl)carbodiimide; “Et₃N” refers to triethylamine; “Et₂O” refers to diethyl ether; “EtOH” refers to ethyl alcohol; “EtOAc” refers to ethyl acetate; “FA” refers to formic acid; “h” refers to hour(s); “HATU” refers to 1-[bis(dimethylamino)methylene]-1H-1,2,3-triazolo[4,5-b]pyridinium 3-oxid hexafluorophosphate; “HCl” refers to hydrochloric acid; “H₂O” refers to water; “Hep” refers to heptane; “Hex” refers to hexane(s); “HOAc” refers to acetic acid; “HOBT” refers to Hydroxybenzotriazole; “HOSu” refers to N-Hydroxysuccinimide; “iPrOH” refers to isopropyl alcohol; “KOAc” refers to potassium acetate; “K₂CO₃” refers to potassium carbonate; “KOH” refers to potassium hydroxide; “Lawesson's reagent” refers to 2,4-Bis(4-methoxyphenyl)-2,4-dithioxo-1,3,2,4-dithiadiphosphetane; “LiOH” refers to lithium hydroxide; “Me” refers to methyl; “MeCN” refers to acetonitrile; “MeOH” refers to methanol; “Mel” refers to methyl iodide; “min” refers to minute(s); “N₂H₄” refers to hydrazine; “NaI” refers to sodium iodide; “NMM” refers to N-Methylmorpholine; “NaOAc” refers to sodium acetate; “NaBH(OAc)₃” refers to sodium triacetoxyborohydride. “N₂” refers to nitrogen; “NaNO₂” refers to sodium nitrite; “Na₂SO₄” refers to sodium sulfate; “NH₃” refers to ammonia; “NH₄OAc” refers to ammonium acetate; “ON” refers to overnight; “PCl₃” refers to Phosphorus trichloride; “PyBOP” refers to Benzotriazole-1-yl-oxytripyrrolidinophosphonium hexafluorophosphate; “Pd/C” refers to Palladium on Carbon; “Pd(OAc)₂” refers to palladium(II) acetate; “PdCl₂(PPh₃)₂” refers to bis(triphenylphosphine)palladium(II) dichloride; “Pd₂(dba)₃” refers to tris(dibenzylideneacetone) dipalladium (0); “Pd(dppf)Cl₂” refers to (1,1′-bis(diphenylphosphino)ferrocene)palladium(II) dichloride; “Pd(dppf)Cl₂·DCM” refers to (1,1′-bis(diphenylphosphino)ferrocene)palladium(II) dichloride complex with dichloromethane; “Pd(OH)₂/C” refers to palladium hydroxide on carbon; “PE” refers to petroleum ether; “PPh₃” refers to triphenylphosphine; “PtO2” refers to Platinum(IV)oxide; “sat” refers to saturated; “RT” refers to room temperature; “pTsOH” refers to p-toluenesulfonic acid; “STAB” refers to sodium triacetoxyborohydride; “SnCl₂” refers to tin (II) chloride; “soln” refers to solution; “t-BuBrettPhos Pd G3” refers to [(2-di-tert-butylphosphino-3,6-dimethoxy-2′,4′,6′-triisopropyl-1,1′-biphenyl)-2-(2′-amino-1,1′-biphenyl)]palladium(II) methanesulfonate generation 3; “TBTU” refers to O-(benzotriazol-1-yl)-N,N,N′,N′-tetramethyluronium Tetrafluoroborate or 2-(1H-benzotriazole-1-yl)-1,1,3,3-tetramethylaminium tetrafluoroborate; “TCFH” refers to chloro-N,N,N′,N′-tetramethylformamidinium hexafluorophosphate; “TEA” refers to triethylamine; “TFA” refers to trifluoroacetic acid; “THF” refers to tetrahydrofuran; “TMS” refers to chlorotrimethylsilane; “T3P” refers to propylphosphonic anhydride; “TIPS” refers to triisopropylsilane; “tol” refers to toluene; “XPhos” refers to 2-Dicyclohexylphosphino-2′,4′,6′-triisopropylbiphenyl; “wt.” refers to weight.

### Preparation 1

### tert-Butyl (3-((3-azidopropyl)(methyl)amino)propyl)(methyl)carbamate

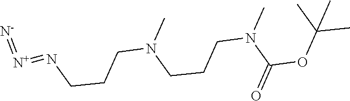 [SMILES: CN(CCCN=[N+]=[N-])CCCN(C)C(=O)OC(C)(C)C]

A soln of 3-azido-N-methylpropan-1-amine (200 mg, 1.75 mmol), Methyl-(3-oxo-propyl)-carbamic acid tert-butyl ester (361 mg, 1.93 mmol) in DCM (10 mL) was treated with STAB (557 mg, 2.63 mmol) and Acetic acid (121 μL, 2.10 mmol). After stirring at RT for 1 h, the mixture was diluted with DCM (10 mL), washed with sat NaHCO₃ (10 mL), extracted with DCM (3×15 ml), dried over Na₂SO₄, filtered, and concentrated to afford title compound (432 mg, 86%). MS ES+ m/z 286 [M+H]⁺.

The following compounds were prepared in a manner essentially analogous to the preparation method of tert-Butyl (3-((3-azidopropyl)(methyl)amino)propyl)(methyl)carbamate using the appropriate reagents, adjusting temperature, adjusting reaction time to determine completion of the reaction, and adjusting the purification system as appropriate.

TABLE 23

| | | | MS |
|---|---|---|---|
| Prep | | | ES+ |
| # | Chemical Name | Structure | m/z |
| 80 | tert-Butyl (3-((3- azidopropyl) (methyl)amino) propyl)carbamate | 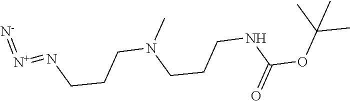 [SMILES: CN(CCCN=[N+]=[N-])CCCNC(=O)OC(C)(C)C] | 272 [M + H]⁺ |

### Preparation 81

### 2-(3-(3-Azidopropoxy)propyl)isoindoline-1,3-dione

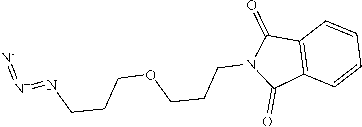 [SMILES: [N-]=[N+]=NCCCOCCCN1C(=O)c2ccccc2C1=O]

A soln of 3-Azido-1-propanol (0.183 mL, 1.98 mmol) in THF (8 mL) at 0° C. was treated with NaH (119 mg, 60% Wt, 2.97 mmol) after 20 min the mixture was treated with 1-Phthalimido-3-bromopropane (530 mg, 1.98 mmol) at 0° C. After stirring for 1 h while warming to RT, the mixture was cooled to 0° C. and treated with aq soln of NH₄Cl (5 mL). The mixture was basified to pH 8-9 using sat aq soln of Na₂CO₃. The mixture was extracted with EtOAc (3 x). The combined organic layer was dried over Na₂SO₄, concentrated and purified by silica gel column chromatography EtOAc/Hep (0 to 100%) to obtain the title compound (434 mg, 76.1%) as a white solid. MS ES+ m/z 311 [M+Na]⁺.

The following compounds were prepared in a manner essentially analogous to the preparation method of 2-(3-(3-Azidopropoxy)propyl)isoindoline-1,3-dione using the appropriate reagents, adjusting temperature, adjusting reaction time to determine completion of the reaction, and adjusting the purification system as appropriate.

TABLE 47

| | | | MS |
|---|---|---|---|
| Prep | | | ES+ |
| # | Chemical Name | Structure | m/z |
| 298 | 2-(3-((3- Hydroxypropyl) (methyl)amino) propyl) isoindoline- 1,3-dione | 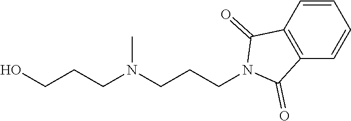 [SMILES: CN(CCCO)CCCN1C(=O)c2ccccc2C1=O] | 277 [M + H]⁺ |

### Preparation 2

### Ethyl 4-((3-((tert-butoxycarbonyl)(methyl)amino)propyl)(methyl)amino)butanoate

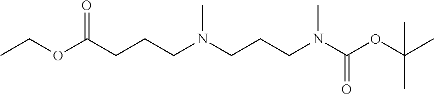 [SMILES: CCOC(=O)CCCN(C)CCCN(C)C(=O)OC(C)(C)C]

A soln of ethyl 4-(methylamino)butanoate hydrochloride (3.96 g, 21.812 mmol) and DIEA (15.38 mg, 0.120 mmol) in MeCN (100 mL) was treated with tert-butyl N-(3-bromopropyl)-N-methylcarbamate (5 g, 19.829 mmol) in portions at RT. After stirring at 80° C. for 16 h, the mixture was concentrated and purified by reversed-phase flash chromatography with the following conditions: column, C18; mobile phase, 10 to 40% ACN in H₂O (10 mmol/L NH₄HCO₃) to afford title compound (1.7 g, 47.81%) as a white liquid. MS ES+ m/z 317 [M+H]⁺.

The following compounds were prepared in a manner essentially analogous to the preparation method of Ethyl 4-((3-((tert-butoxycarbonyl)(methyl)amino)propyl)(methyl)amino)butanoate using the appropriate reagents, adjusting temperature, adjusting reaction time to determine completion of the reaction, and adjusting the purification system as appropriate.

TABLE 1

| | | | MS |
|---|---|---|---|
| Prep | | | ES+ |
| # | Chemical Name | Structure | m/z |
| 3 | tert-Butyl (3-((3- (benzyloxy)propyl) (methyl)amino) propyl)(methyl) carbamate | 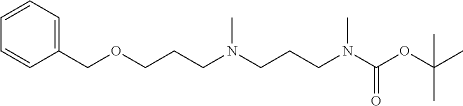 [SMILES: CN(CCCOCc1ccccc1)CCCN(C)C(=O)OC(C)(C)C] | 351 [M + H]⁺ |
| 81 | Benzyl (3-((3- ((tert- butoxycarbonyl) amino)propyl) (methyl)amino) propyl)carbamate | 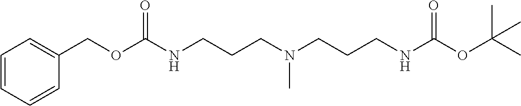 [SMILES: CN(CCCNC(=O)OCc1ccccc1)CCCNC(=O)OC(C)(C)C] | 380 [M + H]⁺ |
| 299 | tert-Butyl 4-(3- ((3- (((benzyloxy) carbonyl)amino) propyl) (methyl)amino) propyl)piperazine- 1-carboxylate | 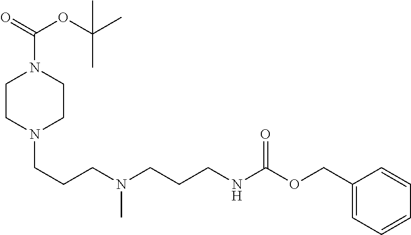 [SMILES: CN(CCCNC(=O)OCc1ccccc1)CCCN1CCN(C(=O)OC(C)(C)C)CC1] | 449 [M + H]⁺ |
| 300 | 3-Bromo-7-(3- bromopropoxy) quinoline | 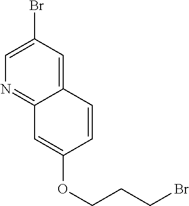 [SMILES: BrCCCOc1ccc2cc(Br)cnc2c1] | 344/346 [M + H]⁺ (⁷⁹Br/⁸¹Br) |
| 301 | tert-Butyl (3-((3- ((3- bromoquinolin-7- yl)oxy)propyl) (methyl)amino) propyl)carbamate | 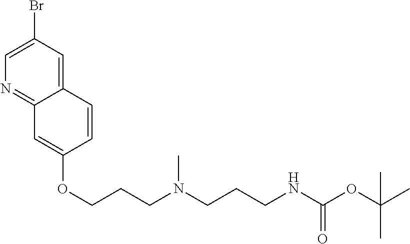 [SMILES: CN(CCCNC(=O)OC(C)(C)C)CCCOc1ccc2cc(Br)cnc2c1] | 452/454 [M + H]⁺ (⁷⁹Br/⁸¹Br) |
| 302 | tert-Butyl (3-((3- (benzyloxy)propyl) (methyl)amino) propyl)carbamate | 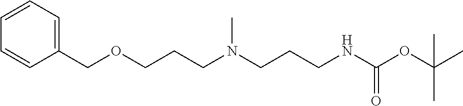 [SMILES: CN(CCCNC(=O)OC(C)(C)C)CCCOCc1ccccc1] | 337 [M + H]⁺ |

### Preparation 4

### 4-((3-((tert-Butoxycarbonyl)(methyl)amino)propyl)(methyl)amino)butanoic acid

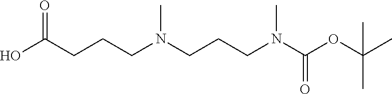 [SMILES: CN(CCCC(=O)O)CCCN(C)C(=O)OC(C)(C)C]

A soln of ethyl 4-({3-[(tert-butoxycarbonyl)(methyl)amino]propyl}(methyl)amino)butanoate (3 g, 9.48 mmol) in THF (30 mL) was treated with LiOH H₂O (0.80 g, 19.07 mmol) in H₂O (6 mL) dropwise at RT. After stirring at 50° C. for 1 h, the resulting mixture was concentrated to afford crude title compound (4 g, >100%). MS ES+ m/z 289 [M+H]⁺.

### Preparation 82

### tert-Butyl (3-((3-(dimethylamino)propyl)amino)propyl)(methyl)carbamate

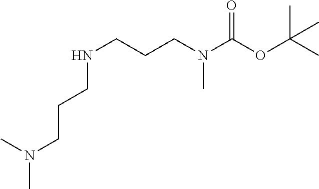 [SMILES: CN(C)CCCNCCCN(C)C(=O)OC(C)(C)C]

A soln of N1,N1-dimethylpropane-1,3-diamine (10.0 g, 97.7 mmol) and tert-butyl N-methyl-N-(3-oxopropyl)carbamate (13.5 g, 72.1 mmol) in EtOH (100 mL) at 0° C. under N₂ was treated with NaBH₄ (7.4 g, 195.7 mmol) in portions over 2 min. After stirring at RT for 16 h, the mixture was cooled to 0° C. and treated with ice water, concentrated and purified by silica gel column chromatography DCM/MeOH (1:99) to obtain the title compound (10 g, 37.4%) as a yellow solid. MS ES+ m/z 274 [M+H]⁺.

The following compounds were prepared in a manner essentially analogous to the preparation method of tert-Butyl (3-((3-(dimethylamino)propyl)amino)propyl)(methyl)carbamate using the appropriate reagents, adjusting temperature, adjusting reaction time to determine completion of the reaction, and adjusting the purification system as appropriate.

TABLE 24

| | | | MS |
|---|---|---|---|
| Prep | | | ES+ |
| # | Chemical Name | Structure | m/z |
| 83 | tert-Butyl methyl(3-(((1- methylpyrrolidin-3- yl)methyl)amino) propyl)carbamate | 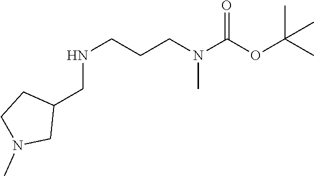 | 462 [M + H]⁺ |

### Preparation 84

### Benzyl 4-((3-((tert-butoxycarbonyl)amino)propyl)(methyl)amino)butanoate

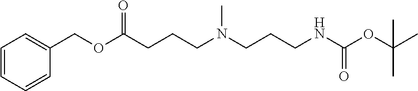 [SMILES: CN(CCCNC(=O)OC(C)(C)C)CCCC(=O)OCc1ccccc1]

A soln of benzyl 4-oxobutanoate (9 g, 46.8 mmol) in DCE (100 mL) was treated with tert-butyl N-[3-(methylamino)propyl]carbamate (9.70 g, 51.5 mmol). After stirring under N₂ at RT for 1 h, the resulting mixture was treated with STAB (19.85 g, 93.65 mmol). After stirring under N₂ at RT for 1 h, the resulting mixture was treated with ice water (10 mL) at 0° C. The resulting mixture was diluted with water (500 mL) and extracted with DCM (3×500 mL). The combined organic layers were washed with brine (2×200 mL), dried over Na₂SO₄, filtered, and concentrated. The residue was purified by silica gel column chromatography DCM/MeOH (10:1) to obtain the title compound (8 g, 46.8%) as a white solid. MS ES+ m/z 365 [M+H]⁺.

The following compounds were prepared in a manner essentially analogous to the preparation method of Benzyl 4-((3-((tert-butoxycarbonyl)amino)propyl)(methyl)amino)butanoate using the appropriate reagents, adjusting temperature, adjusting reaction time to determine completion of the reaction, and adjusting the purification system as appropriate.

TABLE 25

| Prep | | | MS ES+ |
|---|---|---|---|
| # | Chemical Name | Structure | m/z |
| 85 | 2-(1-(3- (((benzyloxy)carbonyl) amino)propyl)piperidin- 4-yl)acetic acid | 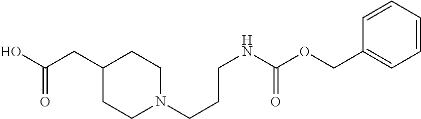 [SMILES: O=C(O)CC1CCN(CCCNC(=O)OCc2ccccc2)CC1] | 335 [M + H]⁺ |
| 86 | Benzyl 4-((3-((tert- butoxycarbonyl)(methyl) amino)propyl)(3- (dimethylamino)propyl) amino)butanoate | 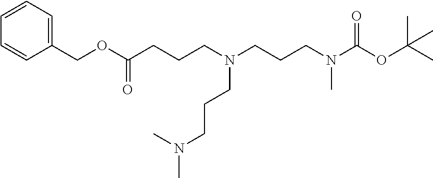 [SMILES: CN(C)CCCN(CCCC(=O)OCc1ccccc1)CCCN(C)C(=O)OC(C)(C)C] | 450 [M + H]⁺ |
| 87 | Benzyl 4-((3-((tert- butoxycarbonyl)(methyl) amino)propyl)((1- methylpyrrolidin-3- yl)methyl)amino) butanoate | 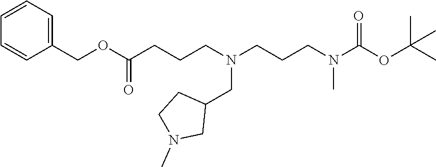 [SMILES: CN1CCC(CN(CCCC(=O)OCc2ccccc2)CCCN(C)C(=O)OC(C)(C)C)C1] | 462 [M + H]⁺ |

### Preparation 88

### N¹,N¹-Dimethyl-N³-(pyridin-2-ylmethyl)propane-1,3-diamine

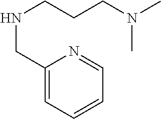

A soln of (3-aminopropyl)dimethylamine (307 μL, 2.45 mmol) and Pyridine-2-carbaldehyde (279 μL, 2.94 mmol) in DCM (10 mL) was treated with STAB (778 mg, 3.67 mmol) and AcOH (154 μL, 2.69 mmol)). After stirring at RT for 6 h, the reaction mixture was concentrated and purified by reversed-phase flash chromatography with the following conditions: column, C18; mobile phase, 0 to 50% DCM in MeOH to afford title compound (218 mg, 46.1%) as a yellow solid. MS ES+ m/z 194 [M+H]⁺.

### Preparation 5

### 4-(Methyl (3-(methylamino)propyl)amino)butanoic acid

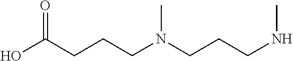

A soln of 4-({3-[(tert-butoxycarbonyl)(methyl)amino]propyl}(methyl)amino)butanoic acid (4 g, 13.870 mmol) and HCl in 1,4-dioxane (4.0 M) (20 mL) was stirred at RT for 6 h before being concentrated to afford crude title compound (6 g, >100%). MS ES+ m/z 189 [M+H]⁺.

The following compounds were prepared in a manner essentially analogous to the preparation method of 4-(Methyl (3-(methylamino)propyl)amino)butanoic acid using the appropriate reagents, adjusting temperature, adjusting reaction time to determine completion of the reaction, and adjusting the purification system as appropriate.

TABLE 2

| Prep | | | MS ES+ |
|---|---|---|---|
| # | Chemical Name | Structure | m/z |
| 6 | N1-(3- (Benzyloxy)propyl)- N1,N3-dimethylpropane- 1,3-diamine | 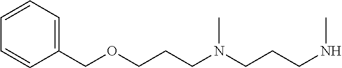 [SMILES: CNCCCN(C)CCCOCc1ccccc1] | 251 [M + H]⁺ |
| 89 | Benzyl 4-((3- aminopropyl)(methyl) amino)butanoate | 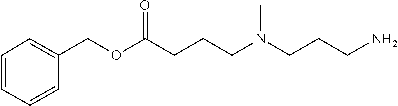 [SMILES: CN(CCCN)CCCC(=O)OCc1ccccc1] | 265 [M + H]⁺ |
| 303 | N1-(3- (Benzyloxy)propyl)-N1- methylpropane-1,3- diamine | 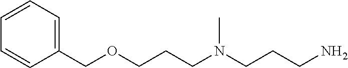 [SMILES: CN(CCCN)CCCOCc1ccccc1] | 237 [M + H]⁺ |

### Preparation 90

### Benzyl (3-((3-aminopropyl)(methyl)amino)propyl)carbamate

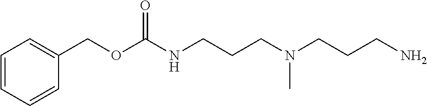 [SMILES: CN(CCCN)CCCNC(=O)OCc1ccccc1]

A soln of benzyl N-[3-({3-[(tert-butoxycarbonyl)amino]propyl}(methyl)amino)propyl]carbamate (12 g, 31.62 mmol) in DCM (120 mL) was treated dropwise with TFA (30 mL). After stirring ON at RT under N₂, the mixture was concentrated to obtain the title crude compound (12 g). MS ES+ m/z 280 [M+H]⁺.

The following compounds were prepared in a manner essentially analogous to the preparation method of Benzyl (3-((3-aminopropyl)(methyl)amino)propyl)carbamate using the appropriate reagents, adjusting temperature, adjusting reaction time to determine completion of the reaction, and adjusting the purification system as appropriate.

TABLE 26

| Prep | | | MS ES+ |
|---|---|---|---|
| # | Chemical Name | Structure | m/z |
| 91 | Benzyl 4-((3- (dimethylamino)propyl) (3- (methylamino)propyl) amino)butanoate | 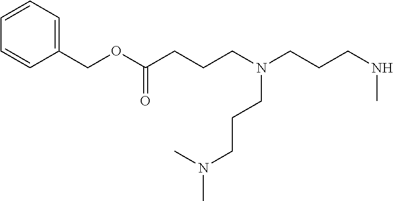 [SMILES: CNCCCN(CCCC(=O)OCc1ccccc1)CCCN(C)C] | 350 [M + H]⁺ |
| 92 | Benzyl 4-((3- (methylamino)propyl) ((1-methylpyrrolidin-3- yl)methyl)amino) butanoate | 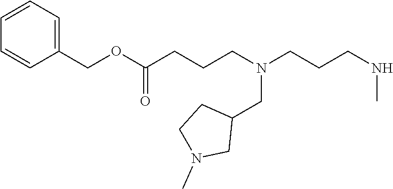 [SMILES: CNCCCN(CCCC(=O)OCc1ccccc1)CC1CCN(C)C1] | 362 [M + H]⁺ |
| 304 | Benzyl (3-(methyl(3- (piperazin-1- yl)propyl)amino)propyl) carbamate | 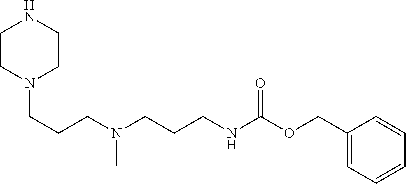 [SMILES: CN(CCCNC(=O)OCc1ccccc1)CCCN1CCNCC1] | 349 [M + H]⁺ |

### Preparation 93

### (5r,7r)-2-(1-Benzyl-1H-tetrazol-5-yl)adamantan-2-amine

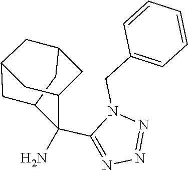 [SMILES: NC1(c2nnnn2Cc2ccccc2)C2CC3CC(C2)CC1C3]

A soln of adamantanone (300 mg, 1.997 mmol) and NH₄Cl (160 mg, 2.996 mmol) in MeOH (3 mL) and H₂O (1 mL) at RT under N₂ was treated with (isocyanomethyl)benzene (234 mg, 1.997 mmol) and NaN₃ (195 mg, 2.996 mmol). After stirring ON, the mixture was filtered, and the filter cake washed with H₂O (3×5 mL). The combined filtrates were concentrated under reduced pressure to afford crude title compound 2-(1-benzyl-1,2,3,4-tetrazol-5-yl)adamantan-2-amine (590 mg, 95.4%) as a white solid. MS ES+ m/z 310 [M+H]⁺.

### Preparation 94

### 3-(3-Iodo-1H-indazol-1-yl)-N,N-dimethylpropan-1-amine

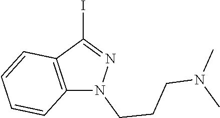 [SMILES: CN(C)CCCn1nc(I)c2ccccc21]

A soln of 3-iodo-1H-indazole (200 mg, 0.820 mmol) in DMF (4 mL) was treated with potassium tert-butoxide (138 mg, 1.23 mmol). After stirring at RT for 30 min, the mixture was treated with a soln of 3-chloro-N,N-dimethylpropan-1-amine (120 mg, 0.983 mmol) in DMF (1 mL). After stirring at RT for 23 h, the reaction was filtered, and directly purified by reversed-phase flash chromatography with the following conditions: column, C18; mobile phase, 30% to 50% ACN in MeOH (5% 10 mM NH₄HCO₃) to afford title compound (98 mg, 36%) as a white solid. MS ES+ m/z 330 [M+H]⁺.

### Preparation 95

### 2-(2-((2-Hydroxyethyl)(methyl)amino)ethyl)isoindoline-1,3-dione

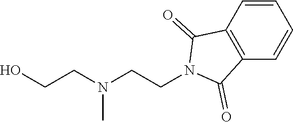 [SMILES: CN(CCO)CCN1C(=O)c2ccccc2C1=O]

A soln of 2-[(2-aminoethyl)methylamino]Ethanol (400.0 mg, 3.385 mmol) in 2-Methyltetrahydrofuran (3 mL) was treated with Phthalic anhydride (360 μL, 3.723 mmol) and TEA (1.41 mL, 10.15 mmol) at RT. After stirring at 85° C. for 3 h, the mixture was concentrated. The residue was diluted with saturated aq Na₂CO₃ soln (5 mL), extracted with EtOAc (2×10 mL), dried over anhydrous Na₂SO₄, and concentrated to afford crude title compound (570 mg, 67.8%) as a light yellow solid. MS ES+ m/z 249 [M+H]⁺.

### Preparation 96

### 3-Bromo-N-(3-(dimethylamino)propyl)-N-methylquinoline-7-carboxamide

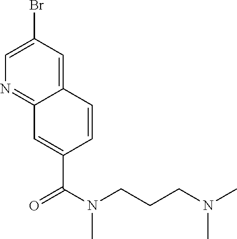 [SMILES: CN(C)CCCN(C)C(=O)c1ccc2cc(Br)cnc2c1]

A soln of 3-bromoquinoline-7-carboxylic acid (100 mg, 0.397 mmol) in DCM (2 mL) was treated with DIPEA (0.276 mL, 1.59 mmol) and HATU (151 mg, 0.397 mmol). After stirring for 3 min at RT, the mixture was treated with N,N,N′-trimethyl-1,3-propanediamine (0.116 mL, 0.793 mmol). After stirring at RT for 1 h, the reaction mixture was dried under N₂, dissolved in DMSO (2 mL) and purified reversed-phase flash chromatography with the following conditions: column, C18; mobile phase, 10 to 100% ACN in MeOH (10 mM NH₄HCO₃) to afford title compound (113 mg, 81.3%) as a colorless oil. MS ES+ m/z 350/352 [M+H]⁺ (¹⁹Br/⁸¹Br).

The following compounds were prepared in a manner essentially analogous to the preparation method of 3-Bromo-N-(3-(dimethylamino)propyl)-N-methylquinoline-7-carboxamide using the appropriate reagents, adjusting temperature, adjusting reaction time to determine completion of the reaction, and adjusting the purification system as appropriate.

TABLE 27

| Prep | | | MS ES+ |
|---|---|---|---|
| # | Chemical Name | Structure | m/z |
| 97 | 4-Bromo-2-cyano-N-(3- (dimethylamino)propyl)- N-methylbenzamide | 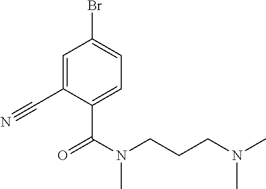 [SMILES: CN(C)CCCN(C)C(=O)c1ccc(Br)cc1C#N] | 324/326 [M + H]⁺ (⁷⁹Br/⁸¹Br) |
| 98 | 4-Bromo-N-(3- (dimethylamino)propyl)- N-methyl-2- nitrobenzamide | 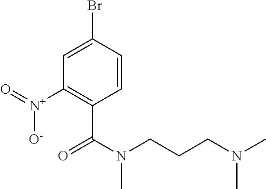 [SMILES: CN(C)CCCN(C)C(=O)c1ccc(Br)cc1[N+](=O)[O-]] | 344/346 [M + H]⁺ (⁷⁹Br/⁸¹Br) |
| 99 | 6-Bromo-N-(3- (dimethylamino)propyl)- N-methyl-2- naphthamide | 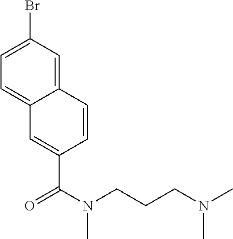 [SMILES: CN(C)CCCN(C)C(=O)c1ccc2cc(Br)ccc2c1] | 349/351 [M + H]⁺ (⁷⁹Br/⁸¹Br) |
| 100 | 4-Bromo-N-(3- (dimethylamino)propyl)- 2-methoxy-N- methylbenzamide | 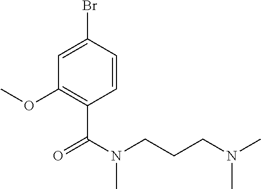 | 329/331 [M + H]⁺ (⁷⁹Br/⁸¹Br) |
| 101 | 7-Bromo-N-(3- (dimethylamino)propyl)- N-methylquinoline-3- carboxamide | 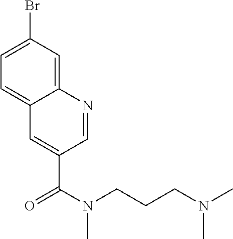 [SMILES: CN(C)CCCN(C)C(=O)c1cnc2cc(Br)ccc2c1] | 350/352 [M + H]⁺ (⁷⁹Br/⁸¹Br) |

### Preparation 102

### N-(4-(Benzyloxy)phenyl)-4-(dimethylamino)butanamide

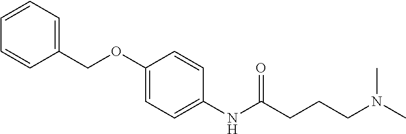 [SMILES: CN(C)CCCC(=O)Nc1ccc(OCc2ccccc2)cc1]

A soln of 4-(dimethylamino)butanoic acid hydrochloride (3 g, 17.897 mmol) in ACN (50 mL) was treated with TCFH (7.53 g, 26.845 mmol) and 1-methyl-1H-imidazole (2.94 g, 35.794 mmol). After stirring at RT under N₂ for 30 min, the mixture was treated with 4-(benzyloxy) aniline (4.28 g, 21.476 mmol). After stirring at RT for 2 h, the mixture was diluted with H₂O (100 mL) and extracted with EtOAc (3×150 mL). The combined organic layers were washed with brine (3×200 mL), dried over Na₂SO₄, filtered, concentrated and purified by reversed-phase flash chromatography with the following conditions: column, C18; mobile phase, 50 to 70% ACN in H₂O (0.1% FA) to afford title compound (3.5 g, 62.6%) as a yellow solid. MS ES+ m/z 313 [M+H]⁺.

The following compounds were prepared in a manner essentially analogous to the preparation method of N-(4-(Benzyloxy)phenyl)-4-(dimethylamino)butanamide using the appropriate reagents, adjusting temperature, adjusting reaction time to determine completion of the reaction, and adjusting the purification system as appropriate.

TABLE 28

| Prep | | | MS ES+ |
|---|---|---|---|
| # | Chemical Name | Structure | m/z |
| 103 | tert-Butyl 4-((4-(4- iodophenyl)butanamido) methyl)benzoate | 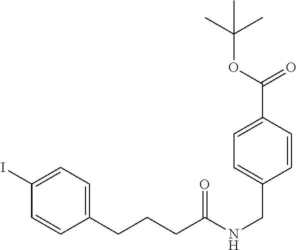 [SMILES: CC(C)(C)OC(=O)c1ccc(CNC(=O)CCCc2ccc(I)cc2)cc1] | 480 [M + H]⁺ |

### Preparation 104

### 4-((4-(4-Iodophenyl)butanamido)methyl)benzoic acid

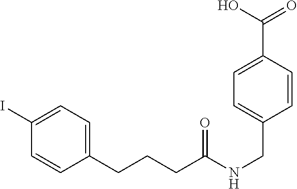 [SMILES: O=C(CCCc1ccc(I)cc1)NCc1ccc(C(=O)O)cc1]

A soln of tert-butyl 4-{[4-(4-iodophenyl)butanamido]methyl}benzoate (33 g, 68.842 mmol) in DCM (300 mL) was treated with HCl in 1,4-dioxane (4.0 M) (86 mL, 344.210 mmol) in portions at RT under N₂. After stirring at 50° C. for 4 h the mixture was concentrated to afford crude title compound (30 g) as a white solid. MS ES+ m/z 424 [M+H]⁺.

### Preparation 105

### tert-Butyl N6-(((9H-fluoren-9-yl)methoxy)carbonyl)-N2-(4-((4-(4-iodophenyl)butanamido)methyl)benzoyl)-L-lysinate

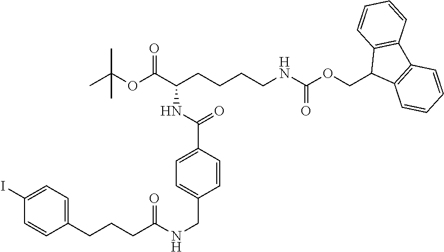 [SMILES: CC(C)(C)OC(=O)[C@H](CCCCNC(=O)OCC1c2ccccc2-c2ccccc21)NC(=O)c1ccc(CNC(=O)CCCc2ccc(I)cc2)cc1]

A soln of 4-{[4-(4-iodophenyl)butanamido]methyl}benzoic acid (30 g, 70.880 mol) and NMM (21.51 g, 212.640 mmol) in DMF (300 mL) was treated with CDMT (14.93 g, 85.056 mmol) in portions at RT under N₂. After stirring for 30 min the mixture was treated with tert-butyl(2S)-2-amino-6-{[(9H-fluoren-9-ylmethoxy)carbonyl]amino}hexanoate (36.11 g, 85.056 mmol). After stirring for 4 h the mixture was diluted with EtOAc (500 mL). The combined organic layers were washed with brine (3×300 mL), dried over Na₂SO₄, filtered, concentrated and purified by reversed-phase flash chromatography with the following conditions: column, C18; mobile phase, 50 to 70% ACN in H₂O (0.1% FA) to afford title compound (60 g) as a light yellow solid. MS ES+ m/z 830 [M+H]⁺.

### Preparation 106

### N6-(((9H-Fluoren-9-yl)methoxy)carbonyl)-N2-(4-((4-(4-iodophenyl)butanamido)methyl)benzoyl)-L-lysine

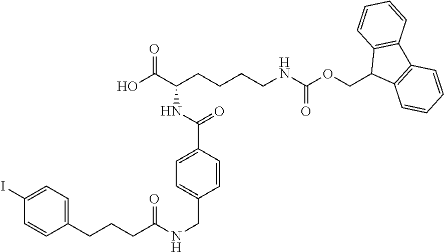 [SMILES: O=C(CCCc1ccc(I)cc1)NCc1ccc(C(=O)N[C@@H](CCCCNC(=O)OCC2c3ccccc3-c3ccccc32)C(=O)O)cc1]

A soln of tert-butyl(2S)-6-{[(9H-fluoren-9-ylmethoxy)carbonyl]amino}-2-[(4-{[4-(4-iodophenyl)butanamido]methyl}phenyl)formamido]hexanoate (20 g, 24.103 mmol) in DCM (200 mL) was treated with HCl in 1,4-dioxane (4.0 M) (12 mL, 48.206 mmol) at RT under N₂. After stirring for ON, the mixture was concentrated to afford crude title compound (18 g, 96.0%) as a yellow solid. MS ES+ m/z 774 [M+H]⁺.

### Preparation 107

### tert-Butyl rac-4-(6-((((9H-fluoren-9-yl)methoxy)carbonyl)amino)-2-(4-((4-(4-iodophenyl)butanamido)methyl)benzamido)hexanamido)butanoate

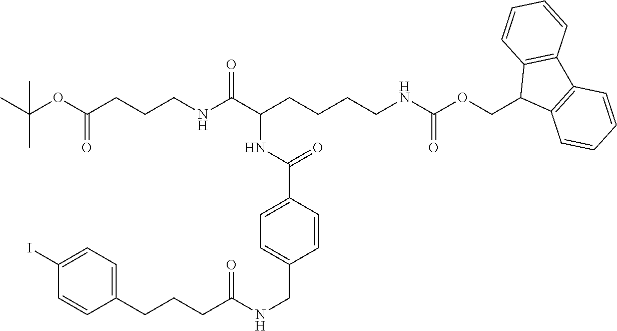 [SMILES: CC(C)(C)OC(=O)CCCNC(=O)C(CCCCNC(=O)OCC1c2ccccc2-c2ccccc21)NC(=O)c1ccc(CNC(=O)CCCc2ccc(I)cc2)cc1]

A soln of (2S)-6-{[(9H-fluoren-9-ylmethoxy)carbonyl]amino}-2-[(4-{[4-(4-iodophenyl)butanamido]methyl}phenyl)formamido]hexanoic acid (16 g, 20.681 mmol) in ACN (200 mL) was treated with 1-methyl-1H-imidazole (5.09 g, 62.043 mmol) and TCFH (8.70 g, 31.022 mmol) at RT under N₂. After stirring for 30 min the mixture was treated with tert-butyl 4-aminobutanoate (4.94 g, 31.022 mmol). After stirring at 50° C. for 16 h the mixture was diluted with H₂O (500 mL) and extracted with EtOAc (3×300 mL). The combined organic layers were washed with brine (1×100 mL), dried over Na₂SO₄, filtered, concentrated, and purified by reversed-phase flash chromatography with the following conditions: column, C18; mobile phase, 50 to 70% ACN in H₂O (0.1% FA) to afford title compound (9.6 g, 50.7%) as a white solid. MS ES+ m/z 915 [M+H]⁺.

### Preparation 108

### rac-4-(6-((((9H-Fluoren-9-yl)methoxy)carbonyl)amino)-2-(4-((4-(4-iodophenyl)butanamido)methyl)benzamido)hexanamido)butanoic acid

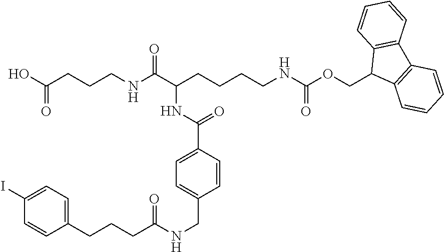 [SMILES: O=C(O)CCCNC(=O)C(CCCCNC(=O)OCC1c2ccccc2-c2ccccc21)NC(=O)c1ccc(CNC(=O)CCCc2ccc(I)cc2)cc1]

A soln of tert-butyl rac-4-(6-((((9H-fluoren-9-yl)methoxy)carbonyl)amino)-2-(4-((4-(4-iodophenyl)butanamido)methyl)benzamido)hexanamido)butanoate (9.6 g, 8.744 mmol) in DCM (50 mL) was treated with HCl in 1,4-dioxane (4.0 M) (10 mL, 0.002 mmol). After stirring at RT ON, the mixture was concentrated to afford crude title compound (8 g, 83.3%) as a yellow solid. MS ES+ m/z 859 [M+H]⁺.

### Preparation 109

### (S)-4-(6-((((9H-Fluoren-9-yl)methoxy)carbonyl)amino)-2-(4-((4-(4-iodophenyl)butanamido)methyl)benzamido)hexanamido)butanoic acid

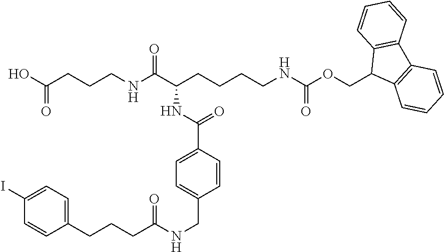 [SMILES: O=C(O)CCCNC(=O)[C@H](CCCCNC(=O)OCC1c2ccccc2-c2ccccc21)NC(=O)c1ccc(CNC(=O)CCCc2ccc(I)cc2)cc1]

Rac-4-(6-((((9H-fluoren-9-yl)methoxy)carbonyl)amino)-2-(4-((4-(4-iodophenyl)butanamido)methyl)benzamido)hexanamido)butanoic acid (8 g) was purified by Prep-SFC with the following conditions: Column, CHIRALPAK IE OBD 3*25 cm, 5 μm; Mobile Phase, 40% DCM/THF (1:1) in EtOH/THF (1:1). The second eluting peak fractions were concentrated and purified by reversed-phase flash chromatography with the following conditions: column, C18; mobile phase, 50 to 70% ACN in H₂O (0.1% FA) to afford title compound (2.008 g, 26.6%) as a yellow solid. MS ES+ m/z 859 [M+H]⁺.

### Preparation 110

### 3-((3-(Dimethylamino)propyl)amino)propanenitrile

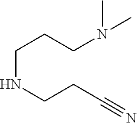

A soln of N1,N1-dimethylpropane-1,3-diamine (6.3 g, 44.039 mmol) in MeOH (40 mL) was treated with acrylonitrile (3.20 mL, 48.443 mmol) at 0° C. After stirring at 0° C. for 2 h the mixture was concentrated to afford crude title compound (7 g, 73.11%) as a colorless liquid. MS ES+ m/z 156 [M+H]⁺.

### Preparation 111

### tert-Butyl(2-cyanoethyl)(3-(dimethylamino)propyl)carbamate

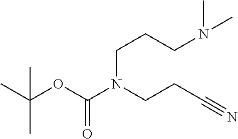 [SMILES: CN(C)CCCN(CCC#N)C(=O)OC(C)(C)C]

A soln of 3-{[3-(dimethylamino)propyl]amino}propanenitrile (7 g, 45.090 mmol) in MeOH (100 mL) was treated with Boc₂O (14.76 g, 67.635 mmol). After stirring at RT ON under N₂ the mixture was concentrated and purified by reversed-phase flash chromatography with the following conditions: column, C18; mobile phase, 10 to 50% ACN in H₂O (0.1% NH₄OH) to afford title compound (6.3 g, 54.71%) as a light yellow liquid. MS ES+ m/z 256 [M+H]⁺.

### Preparation 112

### tert-Butyl (3-aminopropyl)(3-(dimethylamino)propyl)carbamate

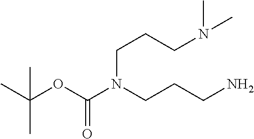 [SMILES: CN(C)CCCN(CCCN)C(=O)OC(C)(C)C]

A soln of Raney nickel (2 g, 34.075 mmol) and NH₄OH (1 mL) in MeOH (50 mL) was treated with tert-butyl N-(2-cyanoethyl)-N-[3-(dimethylamino)propyl]carbamate (6.3 g, 24.671 mmol). After stirring at RT ON under N₂, the mixture was filtered and the filter cake was washed with MeOH (3×20 mL). The filtrate was concentrated and purified by reversed-phase flash chromatography with the following conditions: column, C18; mobile phase, 10 to 50% ACN in H₂O (10 mmol/L NH₄HCO₃) to afford title compound (6.3 g, 98.45%) as a light yellow liquid. MS ES+ m/z 260 [M+H]⁺.

### Preparation 113

### tert-Butyl (3-(6-((((9H-fluoren-9-yl)methoxy)carbonyl)amino)-2-(4-((4-(4-iodophenyl)butanamido)methyl)benzamido)hexanamido)propyl)(3-(dimethylamino)propyl)carbamate

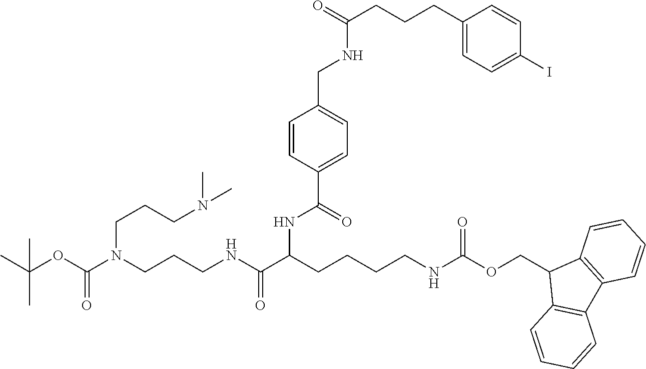 [SMILES: CN(C)CCCN(CCCNC(=O)C(CCCCNC(=O)OCC1c2ccccc2-c2ccccc21)NC(=O)c1ccc(CNC(=O)CCCc2ccc(I)cc2)cc1)C(=O)OC(C)(C)C]

A soln of (2S)-6-{[(9H-fluoren-9-ylmethoxy)carbonyl]amino}-2-[(4-{[4-(4-iodophenyl)butanamido]methyl}phenyl)formamido]hexanoic acid (14 g, 18.096 mmol) in DCM (200 mL) was treated with (3-[[(ethylimino)methylidene]amino]propyl)dimethylamine hydrochloride (5.20 g, 27.144 mmol) and HOBT (4.89 g, 36.192 mmol) at RT. After stirring for 5 min the mixture was treated with tert-butyl N-(3-aminopropyl)-N-[3-(dimethylamino)propyl]carbamate (5.63 g, 21.715 mmol). After stirring for 2 h, the mixture was diluted with H₂O (500 mL) and extracted with EtOAc (2×500 mL). The combined organic layers were dried over Na₂SO₄, acidified to pH 1 with concentrated HCl, filtered, concentrated and purified by reversed-phase flash chromatography with the following conditions: column, C18; mobile phase, 10 to 50% ACN in H₂O (0.1% HCl) to afford title compound (16 g, 67.65%) as a light yellow oil. MS ES+ m/z 1015 [M+H]⁺.

### Preparation 114

### (9H-Fluoren-9-yl)methyl Rac-(6-((3-((3-(dimethylamino)propyl)amino)propyl)amino)-5-(4-((4-(4-iodophenyl)butanamido)methyl)benzamido)-6-oxohexyl)carbamate

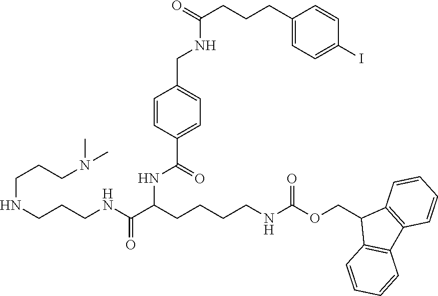 [SMILES: CN(C)CCCNCCCNC(=O)C(CCCCNC(=O)OCC1c2ccccc2-c2ccccc21)NC(=O)c1ccc(CNC(=O)CCCc2ccc(I)cc2)cc1]

A soln of tert-butyl rac-(3-(6-((((9H-fluoren-9-yl)methoxy)carbonyl)amino)-2-(4-((4-(4-iodophenyl)butanamido)methyl)benzamido)hexanamido)propyl)(3-(dimethylamino)propyl)carbamate (16 g, 11.034 mmol) in DCM (200 mL) was treated with HCl in 1,4-dioxane (4.0 M) (50 mL, 200 mmol) at RT. After stirring at RT for 16 h the mixture was concentrated to afford crude title compound (16 g). MS ES+ m/z 916 [M+H]⁺.

### Preparation 115

### (9H-Fluoren-9-yl)methyl(S)—(6-((3-((3-(dimethylamino)propyl)amino)propyl)amino)-5-(4-((4-(4-iodophenyl)butanamido)methyl)benzamido)-6-oxohexyl)carbamate

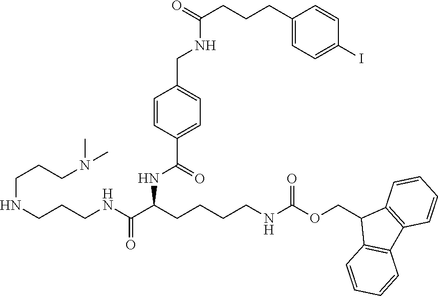 [SMILES: CN(C)CCCNCCCNC(=O)[C@H](CCCCNC(=O)OCC1c2ccccc2-c2ccccc21)NC(=O)c1ccc(CNC(=O)CCCc2ccc(I)cc2)cc1]

(9H-Fluoren-9-yl)methyl Rac-(6-((3-((3-(dimethylamino)propyl)amino)propyl)amino)-5-(4-((4-(4-iodophenyl)butanamido)methyl)benzamido)-6-oxohexyl)carbamate (16 g) was purified by Chiral-HPLC with the following conditions: Column, B_ASA CHIRALPAK IE OBD 5*25 cm, 10 μm; Mobile Phase, 35% MtBE (0.1% DEA) in EtOH (1:1). The second eluting peak fractions were acidified to pH 1 with conc. HCl, concentrated, and purified by reversed-phase flash chromatography with the following conditions: column, C18; mobile phase, 10 to 50% ACN in H₂O (0.1% FA) to afford title compound (3.2 g, 21.3%) as a light yellow solid. MS ES+ m/z 916 [M+H]⁺.

### Preparation 116

### 4-(Dimethylamino)-N-(4-nitrophenyl)butanamide

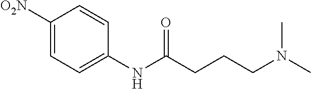 [SMILES: CN(C)CCCC(=O)Nc1ccc([N+](=O)[O-])cc1]

A soln of p-nitroaniline (1.65 g, 11.93 mmol) in pyridine (20 mL) at RT under N₂ was treated with PCl₃ (0.82 g, 5.966 mmol). After stirring for 1 h, the mixture was treated with 4-(dimethylamino)butanoic acid hydrochloride (1 g, 5.96 mmol). After stirring at 50° C. for an additional 3 h, the mixture was diluted with DCM (400 mL) and washed with 1M HCl (3×200 mL). The combined acidic layers were basified with 1M NaOH and extracted with CH₂Cl₂ (3×400 mL). The combined organic layers were washed with brine (1×500 mL), dried over Na₂SO₄, filtered, concentrated and purified by reversed-phase flash chromatography with the following conditions: column, C18; mobile phase, 30% to 50% ACN in H₂O (10 mmol/L NH₄HCO₃) to afford title compound (620 mg, 41.3%) as a yellow solid. MS ES+ m/z 252 [M+H]⁺.

### Preparation 117

### N-(4-Aminophenyl)-4-(dimethylamino)butanamide

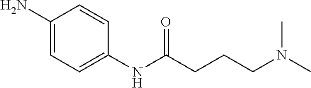 [SMILES: CN(C)CCCC(=O)Nc1ccc(N)cc1]

A soln of 4-(dimethylamino)-N-(4-nitrophenyl)butanamide (500 mg, 1.990 mmol) in EtOAc (6 mL) at RT under N₂ was treated with 10 wt. % Pd/C (424 mg). After stirring for 4 h under H₂, the mixture was filtered, the filter cake washed with EtOAc (6 mL) (3×5 mL), and the filtrate concentrated to afford crude title compound (400 mg, 90.8%) as a light yellow oil. MS ES+ m/z 222 [M+H]⁺.

### Preparation 118

### 4-(Dimethylamino)-N-(4-hydroxyphenyl)butanamide

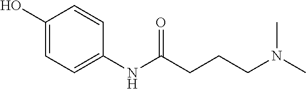 [SMILES: CN(C)CCCC(=O)Nc1ccc(O)cc1]

A soln of N-[4-(benzyloxy)phenyl]-4-(dimethylamino)butanamide (3 g, 9.603 mmol) in ethyl acetate (30 mL) was treated with 10 wt. % Pd/C (1.02 g). After stirring at RT under H₂ ON, the mixture was filtered and the filter cake washed with EtOAc (3×30 mL). The combined organic layers were washed with brine (2×100 mL), dried over Na₂SO₄, filtered, and concentrated to afford crude title compound (1.8 g, 84.3%) as a brown oil. ES+ m/z 223 [M+H]⁺.

### Preparation 7

### 4-(N-methyl-2-(4,7,10-tris(2-(tert-butoxy)-2-oxoethyl)-1,4,7,10-tetraazacyclododecan-1-yl)acetamido)butanoic acid

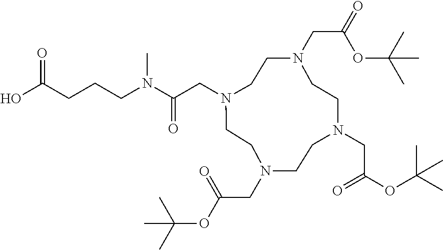 [SMILES: CN(CCCC(=O)O)C(=O)CN1CCN(CC(=O)OC(C)(C)C)CCN(CC(=O)OC(C)(C)C)CCN(CC(=O)OC(C)(C)C)CC1]

A soln of tert-butyl 2-{4,7-bis[2-(tert-butoxy)-2-oxoethyl]-10-{2-[(2,5-dioxopyrrolidin-1-yl)oxy]-2-oxoethyl}-1,4,7,10-tetraazacyclododecan-1-yl}acetate (4.00 g, 5.97 mmol) and 4-(methylamino)butyric acid hydrochloride (1.38 g, 8.95 mmol) in DCM (70 mL) was treated with DIEA (3.09 g, 23.88 mmol). After stirring under N₂ at RT for 2 h the mixture was concentrated under reduced pressure and purified by reversed-phase flash chromatography with the following conditions: column, C18; mobile phase, 30% to 50% ACN in H₂O (0.1% FA) to afford title compound (2.211 g, 51.5%) as a white solid. MS ES+ m/z 672 [M+H]⁺.

The following compounds were prepared in a manner essentially analogous to the preparation method of 4-(N-methyl-2-(4,7,10-tris(2-(tert-butoxy)-2-oxoethyl)-1,4,7,10-tetraazacyclododecan-1-yl)acetamido)butanoic acid using the appropriate reagents, adjusting temperature, adjusting reaction time to determine completion of the reaction, and adjusting the purification system as appropriate.

TABLE 3

| Prep | | | MS ES+ |
|---|---|---|---|
| # | Chemical Name | Structure | m/z |
| 7 | 4-(Methyl(3-(N-methyl- 2-(4,7,10-tris(2-(tert- butoxy)-2-oxoethyl)- 1,4,7,10- tetraazacyclododecan-1- yl)acetamido)propyl) amino)butanoic acid | 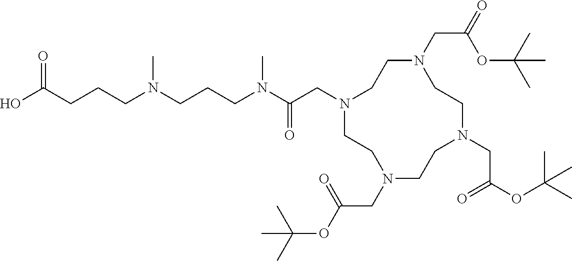 [SMILES: CN(CCCC(=O)O)CCCN(C)C(=O)CN1CCN(CC(=O)OC(C)(C)C)CCN(CC(=O)OC(C)(C)C)CCN(CC(=O)OC(C)(C)C)CC1] | 744 [M + H]⁺ |
| 8 | tri-tert-Butyl 2,2′,2″-(10- (2-((3-((3- (benzyloxy)propyl) (methyl)amino)propyl) (methyl)amino)-2- oxoethyl)-1,4,7,10- tetraazacyclododecane- 1,4,7-triyl)triacetate | 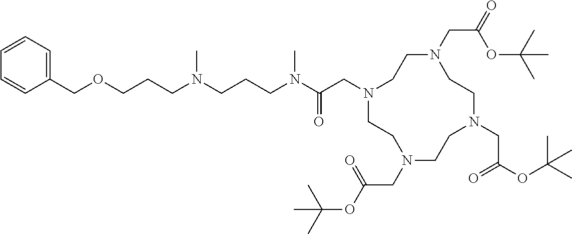 [SMILES: CN(CCCOCc1ccccc1)CCCN(C)C(=O)CN1CCN(CC(=O)OC(C)(C)C)CCN(CC(=O)OC(C)(C)C)CCN(CC(=O)OC(C)(C)C)CC1] | 806 [M + H]⁺ |
| 119 | tri-tert-Butyl 2,2′,2″-(10- (2-((3-((4-(benzyloxy)- 4- oxobutyl)(methyl)amino) propyl)amino)-2- oxoethyl)-1,4,7,10- tetraazacyclododecane- 1,4,7-triyl)triacetate | 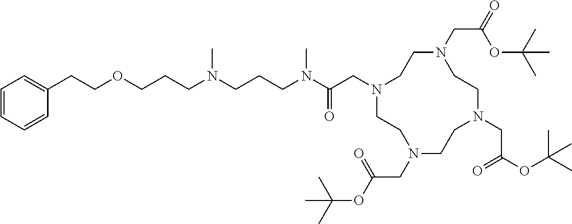 [SMILES: CN(CCCOCCc1ccccc1)CCCN(C)C(=O)CN1CCN(CC(=O)OC(C)(C)C)CCN(CC(=O)OC(C)(C)C)CCN(CC(=O)OC(C)(C)C)CC1] | 820 [M + H]⁺ |
| 120 | tri-tert-Butyl 2,2′,2″-(10- (8-methyl-3,13-dioxo-1- phenyl-2-oxa-4,8,12- triazatetradecan-14-yl)- 1,4,7,10- tetraazacyclododecane- 1,4,7-triyl)triacetate | 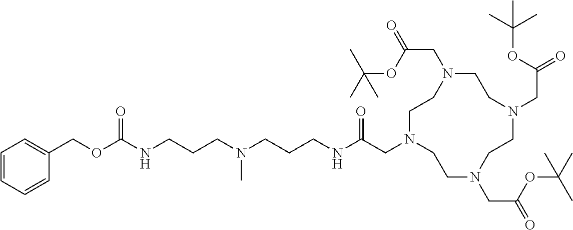 [SMILES: CN(CCCNC(=O)CN1CCN(CC(=O)OC(C)(C)C)CCN(CC(=O)OC(C)(C)C)CCN(CC(=O)OC(C)(C)C)CC1)CCCNC(=O)OCc1ccccc1] | 835 [M + H]⁺ |
| 121 | tri-tert-Butyl 2,2′,2″-(10- (2-((3-((4-(benzyloxy)- 4-oxobutyl)(3- (dimethylamino)propyl) amino)propyl)(methyl) amino)-2-oxoethyl)- 1,4,7,10- tetraazacyclododecane- 1,4,7-triyl)triacetate | 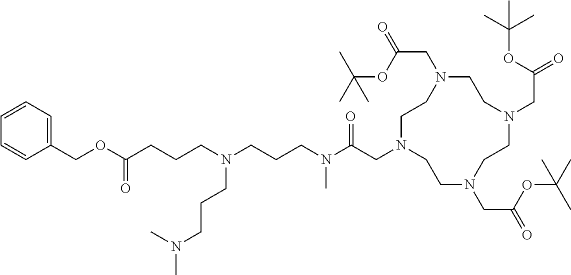 [SMILES: CN(C)CCCN(CCCC(=O)OCc1ccccc1)CCCN(C)C(=O)CN1CCN(CC(=O)OC(C)(C)C)CCN(CC(=O)OC(C)(C)C)CCN(CC(=O)OC(C)(C)C)CC1] | 905 [M + H]⁺ |
| 122 | tri-tert-Butyl 2,2′,2″-(10- (2-((3-((4-(benzyloxy)- 4-oxobutyl)((1- methylpyrrolidin-3- yl)methyl)amino)propyl) (methyl)amino)-2- oxoethyl)-1,4,7,10- tetraazacyclododecane- 1,4,7-triyl)triacetate | 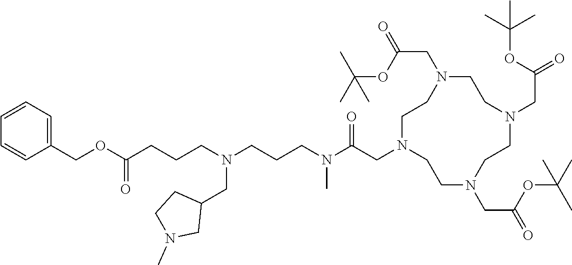 [SMILES: CN1CCC(CN(CCCC(=O)OCc2ccccc2)CCCN(C)C(=O)CN2CCN(CC(=O)OC(C)(C)C)CCN(CC(=O)OC(C)(C)C)CCN(CC(=O)OC(C)(C)C)CC2)C1] | 917 [M + H]⁺ |
| 305 | tri-tert-Butyl 2,2′,2″-(10- (2-((3-((3-(benzyloxy) propyl)(methyl) amino)propyl)amino)- 2-oxoethyl)-1,4,7,10- tetraazacyclododecane- 1,4,7-triyl)triacetate | 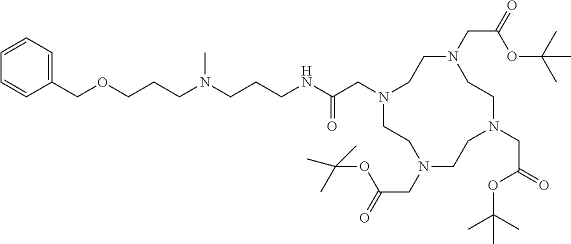 [SMILES: CN(CCCNC(=O)CN1CCN(CC(=O)OC(C)(C)C)CCN(CC(=O)OC(C)(C)C)CCN(CC(=O)OC(C)(C)C)CC1)CCCOCc1ccccc1] | 792 [M + H]⁺ |

### Preparation 9

### tri-tert-Butyl 2,2′,2″-(10-(2-(methyl(3-(methylamino)propyl)amino)-2-oxoethyl)-1,4,7,10-tetraazacyclododecane-1,4,7-triyl)triacetate

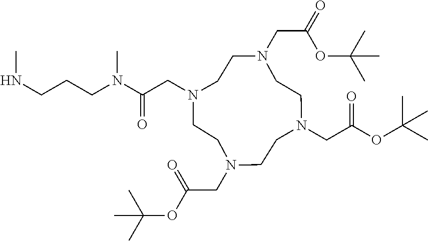 [SMILES: CNCCCN(C)C(=O)CN1CCN(CC(=O)OC(C)(C)C)CCN(CC(=O)OC(C)(C)C)CCN(CC(=O)OC(C)(C)C)CC1]

A soln of N1,N3-dimethylpropane-1,3-diamine (1 g, 0.01 mol), 2-(4,7,10-tris(2-(tert-butoxy)-2-oxoethyl)-1,4,7,10-tetraazacyclododecan-1-yl)acetic acid (3 g, 5 mmol) and N-ethyl-N-isopropylpropan-2-amine (3 g, 0.02 mol) in DMF (8 mL) was treated with T3P (7 g, 50% Wt, 0.01 mol) at 25° C. After stirring at RT for 16 h the mixture was concentrated under vacuum and purified by reversed-phase flash chromatography with the following conditions: column, C18; mobile phase, 0% to 30% ACN in H₂O (0.1% FA) to afford title compound (1.2 g, 30%) as a light yellow oil. MS ES+ m/z 658 [M+H]⁺.

### Preparation 10

### tri-tert-Butyl 2,2′,2″-(10-(2-((3-((3-hydroxypropyl)(methyl)amino)propyl)(methyl)amino)-2-oxoethyl)-1,4,7,10-tetraazacyclododecane-1,4,7-triyl)triacetate

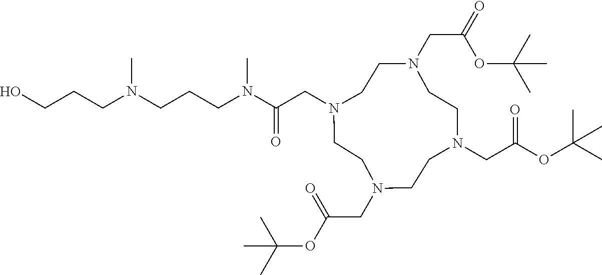 [SMILES: CN(CCCO)CCCN(C)C(=O)CN1CCN(CC(=O)OC(C)(C)C)CCN(CC(=O)OC(C)(C)C)CCN(CC(=O)OC(C)(C)C)CC1]

A soln of tert-butyl 2-(4-{[(3-{[3-(benzyloxy)propyl](methyl)amino}propyl)(methyl)carbamoyl]methyl}-7,10-bis[2-(tert-butoxy)-2-oxoethyl]-1,4,7,10-tetraazacyclododecan-1-yl)acetate (2 g, 2.484 mmol) in EtOH (100 mL) under nitrogen at RT was treated with 20 wt. % Pd(OH) 2/C (50% H₂O) (700 mg). After stirring at 80° C. for 13 h under H₂ (1.0 MPa), the mixture was filtered and the filter cake washed with EtOH (3×20 mL). The filtrate was concentrated and purified by Prep-HPLC with the following conditions: Column, Xtimate C18 OBD 50*250 mm, 10 μm; Mobile Phase, 10 to 40% MeOH in H₂O (0.1% FA) to afford title compound (298 mg, 16%) as a white solid. MS ES+ m/z 716 [M+H]⁺.

The following compounds were prepared in a manner essentially analogous to the preparation method of tri-tert-Butyl 2,2′,2″-(10-(2-((3-((3-hydroxypropyl)(methyl)amino)propyl)(methyl)amino)-2-oxoethyl)-1,4,7,10-tetraazacyclododecane-1,4,7-triyl)triacetate using the appropriate reagents, adjusting temperature, adjusting reaction time to determine completion of the reaction, and adjusting the purification system as appropriate.

TABLE 48

| Prep | | | MS ES+ |
|---|---|---|---|
| # | Chemical Name | Structure | m/z |
| 306 | tri-tert-Butyl 2,2′,2″-(10- (2-((3-((3- hydroxypropyl)(methyl) amino)propyl)amino)-2- oxoethyl)-1,4,7,10- tetraazacyclododecane- 1,4,7-triyl)triacetate | 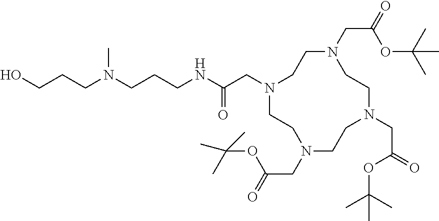 [SMILES: CN(CCCO)CCCNC(=O)CN1CCN(CC(=O)OC(C)(C)C)CCN(CC(=O)OC(C)(C)C)CCN(CC(=O)OC(C)(C)C)CC1] | 702 [M + H]⁺ |

### Preparation 123

### 4-(Methyl (3-(N-methyl-2-(4,7,10-tris(2-(tert-butoxy)-2-oxoethyl)-1,4,7,10-tetraazacyclododecan-1-yl)acetamido)propyl)amino)butanoic acid

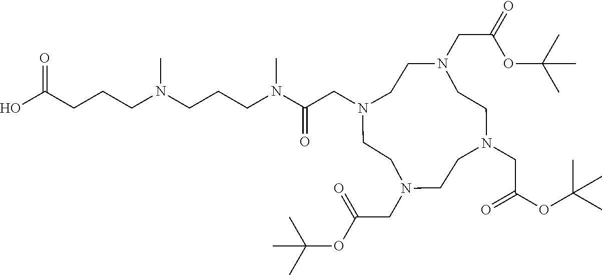 [SMILES: CN(CCCC(=O)O)CCCN(C)C(=O)CN1CCN(CC(=O)OC(C)(C)C)CCN(CC(=O)OC(C)(C)C)CCN(CC(=O)OC(C)(C)C)CC1]

A soln of benzyl 4-{methyl[3-(2-{4,7,10-tris[2-(tert-butoxy)-2-oxoethyl]-1,4,7,10-tetraazacyclododecan-1-yl}acetamido)propyl]amino}butanoate (5.3 g, 6.471 mmol) in MeOH (50 mL) was treated with 20 wt. % Pd(OH) 2/C (2.73 g). After stirring at RT for ON under H₂, the mixture was filtered and the filter cake washed with MeOH (3×21 mL). The filtrate was concentrated to afford crude title compound (3.05 g, 64.6%) as a yellow solid. MS ES+ m/z 730 [M+H]⁺.

### Preparation 124

### 4-((3-(Dimethylamino)propyl)(3-(N-methyl-2-(4,7,10-tris(2-(tert-butoxy)-2-oxoethyl)-1,4,7,10-tetraazacyclododecan-1-yl)acetamido)propyl)amino)butanoic acid

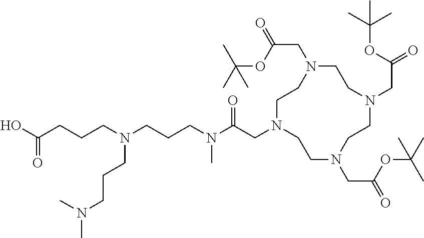 [SMILES: CN(C)CCCN(CCCC(=O)O)CCCN(C)C(=O)CN1CCN(CC(=O)OC(C)(C)C)CCN(CC(=O)OC(C)(C)C)CCN(CC(=O)OC(C)(C)C)CC1]

A soln of 10 wt. % Pd/C (0.82 g) in EtOAc (100 mL) at RT was treated with benzyl 4-{[3-(dimethylamino)propyl][3-(N-methyl-2-{4,7,10-tris[2-(tert-butoxy)-2-oxoethyl]-1,4,7,10-tetraazacyclododecan-1-yl}acetamido)propyl]amino}butanoate (7 g, 7.741 mmol) in portions. After stirring ON under H₂, the mixture was filtered, the filter cake washed with ACN (3×50 mL), and concentrated to afford crude title compound (4.5 g, 65.19%) as a white solid. MS ES+ m/z 815 [M+H]⁺.

The following compounds were prepared in a manner essentially analogous to the preparation method of 4-((3-(Dimethylamino)propyl)(3-(N-methyl-2-(4,7,10-tris(2-(tert-butoxy)-2-oxoethyl)-1,4,7,10-tetraazacyclododecan-1-yl)acetamido)propyl)amino)butanoic acid using the appropriate reagents, adjusting temperature, adjusting reaction time to determine completion of the reaction, and adjusting the purification system as appropriate.

TABLE 29

| Prep | | | MS ES+ |
|---|---|---|---|
| # | Chemical Name | Structure | m/z |
| 125 | 4-((3-(N-Methyl-2- (4,7,10-tris(2-(tert- butoxy)-2-oxoethyl)- 1,4,7,10- tetraazacyclododecan- 1- yl)acetamido)propyl) ((1- methylpyrrolidin-3- yl)methyl)amino) butanoic acid | 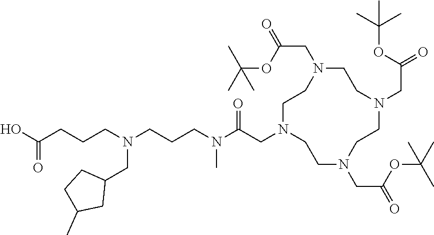 [SMILES: CC1CCC(CN(CCCC(=O)O)CCCN(C)C(=O)CN2CCN(CC(=O)OC(C)(C)C)CCN(CC(=O)OC(C)(C)C)CCN(CC(=O)OC(C)(C)C)CC2)C1] | 827 [M + H]⁺ |

### Preparation 126

### tri-tert-Butyl 2,2′,2″-(10-(2-((3-((3-aminopropyl)(methyl)amino)propyl)amino)-2-oxoethyl)-1,4,7,10-tetraazacyclododecane-1,4,7-triyl)triacetate

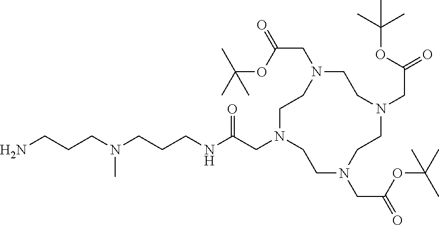 [SMILES: CN(CCCN)CCCNC(=O)CN1CCN(CC(=O)OC(C)(C)C)CCN(CC(=O)OC(C)(C)C)CCN(CC(=O)OC(C)(C)C)CC1]

A soln of tert-butyl 2-{4-[({3-[(3-{[(benzyloxy)carbonyl]amino}propyl)(methyl)amino]propyl}carbamoyl)methyl]-7,10-bis[2-(tert-butoxy)-2-oxoethyl]-1,4,7,10-tetraazacyclododecan-1-yl}acetate (7.1 g, 8.512 mmol) in EtOAc (70 mL) was treated with 10 wt. % Pd/C (2.5 g). After stirring at RT for ON under H₂, the mixture was filtered and the filter cake washed with ACN (3×100 mL). The filtrate was concentrated to afford crude title compound (5.486 g, 92%) as an off-white solid. MS ES+ m/z 701 [M+H]⁺.

### Preparation 388

### 1-(tert-Butyl) 5-(2,5-dioxopyrrolidin-1-yl) 2-(4,7,10-tris(2-(tert-butoxy)-2-oxoethyl)-1,4,7,10-tetraazacyclododecan-1-yl)pentanedioate

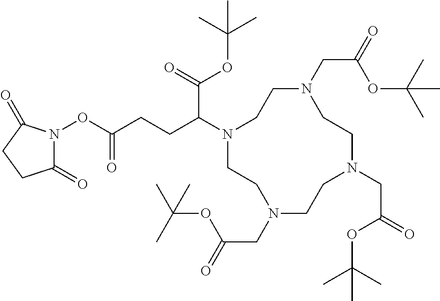 [SMILES: CC(C)(C)OC(=O)CN1CCN(CC(=O)OC(C)(C)C)CCN(C(CCC(=O)ON2C(=O)CCC2=O)C(=O)OC(C)(C)C)CCN(CC(=O)OC(C)(C)C)CC1]

To a soln of rac-(4R)-5-(tert-butoxy)-5-oxo-4-{4,7,10-tris[2-(tert-butoxy)-2-oxoethyl]-1,4,7,10-tetraazacyclododecan-1-yl}pentanoic acid (2 g, 2.85 mmol) and HOSu (656 mg, 5.7 mmol) in DCM (15 mL) at RT under N₂ was treated with DCC (1.18 g, 5.7 mmol). The mixture was stirred ON and concentrated under reduced pressure to afford title compound (2.2 g, 100%). MS ES+ m/z 799 [M+H]⁺.

### Preparation 11

### 4-Iodo-2-isopropylaniline

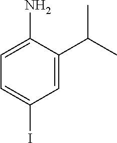

A soln of 2-isopropylaniline (50 g, 369.795 mmol) and Iodine (122.01 g, 480.734 mmol) in HOAc (500 mL) was treated with NaOAc (30.34 g, 369.795 mmol). After stirring under N₂ at RT for 2 h, the resulting mixture was concentrated. The concentrate was diluted with H₂O (500 mL). The resulting mixture was extracted with EA (3×500 mL). The combined organic layers were washed with brine (2×500 mL), dried over anhydrous Na₂SO₄, and concentrated. The residue was purified by silica gel column chromatography PE/EA (10:1) to obtain the title compound as a brown oil (84 g, 87.0%). MS ES+ m/z 262 [M+H]⁺.

### Preparation 127

### 6-Bromo-4-isopropylpyridin-3-amine

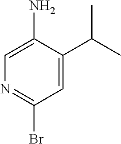

A soln of 4-isopropylpyridin-3-amine (1.03 g, 7.562 mmol) in DMF (50 mL) was cooled in an ice bath and treated with NBS (1.481 g, 8.319 mmol) in 3 portions. After stirring for 1 h the reaction was treated with H₂O (10 mL) and diluted with EtOAc (10 mL). The organic layer was separated, washed with brine, concentrated and purified by reversed-phase flash chromatography with the following conditions: column, C18; mobile phase, 5 to 35% ACN in H₂O (10 mmol/L NH₄HCO₃) to afford title compound (150 mg, 9.2%). MS ES+ m/z 217 [M+H]⁺.

### Preparation 12

### (4-Iodo-2-isopropylphenyl)hydrazine

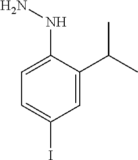

A soln of 4-iodo-2-isopropylaniline (40 g, 153.19 mmol) in HCl (80 mL) was treated with NaNO₂ (21.14 g, 306.38 mmol) in H₂O (40 ml) under N₂ at −10° C. After stirring under N₂ at 0° C. for 1 h SnCl₂·2H₂O (114.07 g, 505.540 mmol) in HCl (40 mL) was added at −10° C. After stirring under N₂ at 0° C. for 1 h the mixture was stirred at RT for an additional 3 h. Precipitated solids were collected by filtration and washed with Et₂O. The residue was triturated with PE/EA (5:1) (200 mL). The resulting mixture was filtered. The filter cake was washed with PE/EA (5:1) (2×50 mL). The filtrate was concentrated to obtain title compound (35 g, crude) as a yellow solid. MS ES+ m/z 277 [M+H]⁺.

The following compounds were prepared in a manner essentially analogous to the preparation method of (4-Iodo-2-isopropylphenyl)hydrazine using the appropriate reagents, adjusting temperature, adjusting reaction time to determine completion of the reaction, and adjusting the purification system as appropriate.

TABLE 4

| Prep | | | MS ES+ |
|---|---|---|---|
| # | Chemical Name | Structure | m/z |
| 13 | (4-bromo-2- isopropylphenyl) hydrazine hydrochloride | 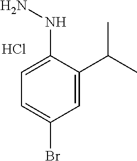 | 230 [M + H]⁺ |
| 14 | 4-Hydrazineyl-3- isopropylbenzoic acid | 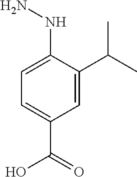 | 195 [M + H]⁺ |
| 15 | (4-Bromo-2-(tert- butyl)phenyl) hydrazine | 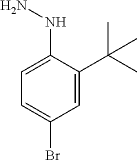 | 243 [M + H]⁺ |
| 128 | 4-Hydrazineyl-3- isopropylbenzoic acid hydrochloride | 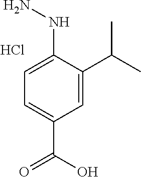 | 195 [M + H]⁺ |
| 129 | 2-Chloro-5- hydrazineyl- 4- isopropylpyridine | 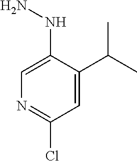 | 186 [M + H]⁺ |
| 130 | 3-Bromo-4- hydrazineyl- benzoic acid | 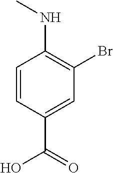 | 231/233 [M + H]⁺ (⁷⁹Br/⁸¹Br) |

### Preparation 131

### 4-(2-(tert-Butoxycarbonyl)hydrazineyl)-3-isopropylbenzoic acid

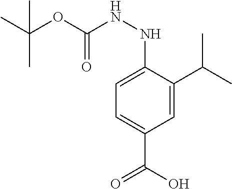 [SMILES: CC(C)c1cc(C(=O)O)ccc1NNC(=O)OC(C)(C)C]

A soln of 4-hydrazineyl-3-isopropylbenzoic acid hydrochloride (4 g, 20.594 mmol) in THF (40 mL) and H₂O (10 mL) was treated with Boc₂O (8.99 g, 41.188 mmol) and NaHCO₃ (3.46 g, 41.188 mmol). After stirring at RT for 1 h under N₂, the mixture was concentrated and purified by silica gel column chromatography PE/EA (5:1) to obtain the title compound (4 g, 65.9%) as a white solid. MS ES+ m/z 295 [M+H]⁺.

### Preparation 132

### tert-Butyl 2-(4-((3-(dimethylamino)propyl)(methyl)carbamoyl)-2-isopropylphenyl)hydrazine-1-carboxylate

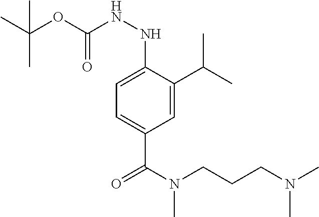 [SMILES: CC(C)c1cc(C(=O)N(C)CCCN(C)C)ccc1NNC(=O)OC(C)(C)C]

A soln of 4-{[(tert-butoxycarbonyl)amino]amino}-3-isopropylbenzoic acid (4 g, 13.589 mmol) in MeCN (50 mL) was treated with [3-(dimethylamino)propyl](methyl)amine (4.74 g, 40.767 mmol), 1-methyl-1H-imidazole (3.35 g, 40.767 mmol), and TCFH (11.44 g, 40.767 mmol). After stirring at RT ON under N₂, was concentrated and purified by reversed-phase flash chromatography with the following conditions: column, C18; mobile phase, 10 to 50% ACN in H₂O (0.1% FA) to afford title compound (4.6 g, 86.2%) as a white solid. MS ES+ m/z 393 [M+H]⁺.

### Preparation 133

### N-(3-(Dimethylamino)propyl)-4-hydrazineyl-3-isopropyl-N-methylbenzamide

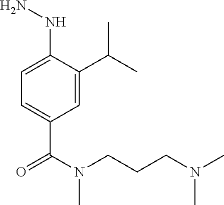 [SMILES: CC(C)c1cc(C(=O)N(C)CCCN(C)C)ccc1NN]

A soln of 4-{[(tert-butoxycarbonyl)amino]amino}-N-[3-(dimethylamino)propyl]-3-isopropyl-N-methylbenzamide (4.6 g, 11.718 mmol) in DCM (30 mL) was treated with HCl in 1,4-dioxane (4.0 M) (20 mL). After stirring at RT for 1 h under N₂, the mixture was concentrated to afford crude title compound (4.3 g) as white solid. MS ES+ m/z 293 [M+H]⁺.

### Preparation 16

### 5-Methoxybenzo[d][1,3]dioxole

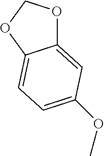

A soln of benzo[d][1,3]dioxol-5-ol (70 g, 506.79 mmol) and K₂CO₃ (210 g, 1520.39 mmol) in acetone (1000 mL) was treated with Mel (143 g, 1013.59 mmol) under N₂ at RT. After stirring under N₂ at 50° C. for 6 h the mixture was cooled to RT and filtered. The filter cake was washed with EA (3×100 mL). The filtrate was concentrated under reduced pressure, diluted with H₂O (1 L), and extracted with EA (3×700 mL). The combined organic layers were washed with brine (1×1000 mL), dried over anhydrous Na₂SO₄, and filtered. The filtrate was concentrated under reduced pressure and purified by silica gel column chromatography PE/EA (20:1) to obtain the title compound (72 g, 93.5%) as a light yellow oil. MS ES+ m/z 153 [M+H]⁺.

### Preparation 134

### 5-(Trifluoromethoxy)benzo[d][1,3]dioxole

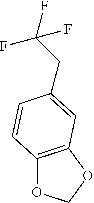 [SMILES: FC(F)(F)Cc1ccc2c(c1)OCO2]

A soln of 4-(Trifluoromethoxy)benzene-1,2-diol (1000 mg, 5.152 mmol) in DMF (10 mL) was treated with Cs₂CO₃ (2.35 g, 7.212 mmol) and Methylene bromide (1.254 g, 7.212 mmol). After heating at 110° C. for 3 h, was cooled, diluted with H₂O (20 mL), and extracted with EtOAc (3×20 mL). The organic layer was separated, washed with brine (20 mL), dried over Na₂SO₄, concentrated under reduced pressure, and purified by silica gel column chromatography hep/EtOAc (0-100%) to obtain the title compound (480 mg, 45.2%) as an oil. 1H NMR (500 MHz, DMSO) δ 7.05 (dt, J=1.7, 0.9 Hz, 1H), 6.99 (d, J=8.5 Hz, 1H), 6.87-6.80 (m, 1H), 6.11 (s, 2H).

The following compounds were prepared in a manner essentially analogous to the preparation method of 5-(Trifluoromethoxy)benzo[d][1,3]dioxole using the appropriate reagents, adjusting temperature, adjusting reaction time to determine completion of the reaction, and adjusting the purification system as appropriate.

TABLE 30

| Prep | | | |
|---|---|---|---|
| # | Chemical Name | Structure | ¹H NMR |
| 135 | Benzo[1,2-d:4,5- d′]bis([1,3]dioxole) | 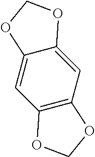 [SMILES: c1c2c(cc3c1OCO3)OCO2] | 1H NMR (400 MHz, DMSO-d6) δ 6.72 (s, 2H), 5.97 (s, 4H) |

### Preparation 17

### 4-Iodo-5-methoxybenzo[d][1,3]dioxole

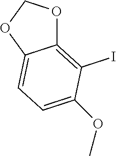

A soln of 5-methoxybenzo[d][1,3]dioxole (70 g, 460.07 mmol) in THF (1000 mL) was treated with n-BuLi (220 mL, 552.09 mmol) under N₂ at −20° C., and stirred for 1 h, before treatment with Iodine (163.48 g, 644.105 mmol). After stirring under N₂ at RT for 30 min the mixture was treated with sat. Na₂S₂O₃ (500 mL) at 0° C. The resulting mixture was diluted with H₂O (1000 mL), extracted with Et₂O Diethyl ether (3×800 mL). The combined organic layers were washed with brine (1×1000 mL), dried over anhydrous Na₂SO₄, and filtered. The filtrate was concentrated under reduced pressure and purified by silica gel column chromatography PE/EA (30:1) to afford title compound (71 g, 55.5%,) as a light yellow solid. MS ES+ m/z 279 [M+H]⁺.

The following compounds were prepared in a manner essentially analogous to the preparation method of 4-Iodo-5-methoxybenzo[d][1,3]dioxole using the appropriate reagents, adjusting temperature, adjusting reaction time to determine completion of the reaction, and adjusting the purification system as appropriate.

TABLE 31

| Prep | | | |
|---|---|---|---|
| # | Chemical Name | Structure | ¹H NMR |
| 136 | 4-Bromo-5- (trifluoromethoxy) benzo[d][1,3]dioxole | 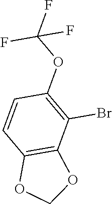 [SMILES: FC(F)(F)Oc1ccc2c(c1Br)OCO2] | (500 MHz, MeOD) δ 6.99-6.82 (m, 2H), 6.14 (s, 2H) |
| 137 | 1-(Benzo[1,2-d:4,5- d′]bis([1,3]dioxole)- 4-yl)ethan-1-one | 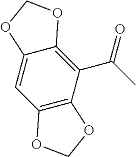 | (300 MHz, DMSO-d6) δ 7.04 (s, 1H), 6.03 (s, 4H), 2.49 (s, 3H) |
| 138 | 1-(5- Bromobenzo[d][1,3] dioxol-4-yl)ethan-1- one | 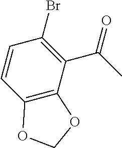 | (400 MHz, DMSO-d6) δ 7.14 (d, 1H), 6.98 (d, 1H), 6.16 (s, 2H), 2.53 (s, 3H). LCMS of 3H-NMR of 3 |

### Preparation 18

### 1-(5-Methoxybenzo[d][1,3]dioxol-4-yl)ethan-1-one

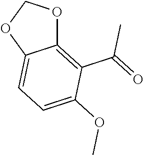

A soln of 4-iodo-5-methoxybenzo[d][1,3]dioxole (70 g, 251.75 mmol) and tributyl(1-ethoxyethenyl) stannane (136.39 g, 377.637 mmol) in Dioxane (800 mL) was treated with Pd(dppf)Cl2 (18.42 g, 25.17 mmol) under N₂ at RT. After stirring under N₂ at 80° C. for 3 h the mixture was treated with HCl in 1,4-dioxane (4.0 M) (95 mL, 377.63 mmol) at 0° C. After stirring at RT for 1 h the mixture was treated with sat. KF (1000 mL) at 0° C. and filtered. The filter cake was washed with EA (3×100 mL), the filtrate concentrated under reduced pressure and purified by silica gel column chromatography PE/EA (4:1) to afford title compound (35 g, 71.5%) as a yellow oil. MS ES+ m/z 195 [M+H]⁺.

### Preparation 19

### Ethyl (Z)-4-hydroxy-4-(5-methoxybenzo[d][1,3]dioxol-4-yl)-2-oxobut-3-enoate

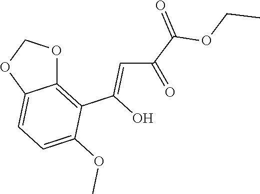 [SMILES: CCOC(=O)C(=O)/C=C(\O)c1c(OC)ccc2c1OCO2]

A soln of 1-(5-methoxybenzo[d][1,3]dioxol-4-yl)ethan-1-one (32 g, 164.79 mmol) in EtOH (400 mL) was treated with sodium ethanolate (112.14 g, 329.58 mmol, 20% in ethanol). After stirring under N₂ at RT for 30 min the mixture was treated with ethyl oxalate (36 g, 247.18 mmol). After stirring at 80° C. overnight the mixture was cooled to RT. Precipitated solids were collected by filtration and washed with H₂O (3×50 mL) to afford title compound (42 g, 86.6%) as a yellow solid. MS ES− m/z 293 [M−H]⁻.

The following compounds were prepared in a manner essentially analogous to the preparation method of Ethyl (Z)-4-hydroxy-4-(5-methoxybenzo[d][1,3]dioxol-4-yl)-2-oxobut-3-enoate using the appropriate reagents, adjusting temperature, adjusting reaction time to determine completion of the reaction, and adjusting the purification system as appropriate.

TABLE 32

| Prep | | | MS ES+ |
|---|---|---|---|
| # | Chemical Name | Structure | m/z |
| 139 | Ethyl (Z)-4-(benzo [1,2-d:4,5- d′]bis([1,3]dioxole)-4- yl)-4-hydroxy-2-oxobut- 3-enoate | 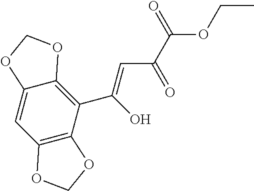 [SMILES: CCOC(=O)C(=O)/C=C(\O)c1c2c(cc3c1OCO3)OCO2] | 307 [M − H]⁻ |
| 140 | Ethyl (Z)-4-(2,6- dimethoxyphenyl)-4- hydroxy-2-oxobut-3-enoate | 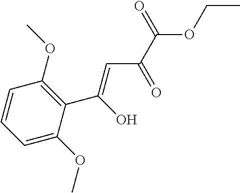 [SMILES: CCOC(=O)C(=O)/C=C(\O)c1c(OC)cccc1OC] | 281 [M + H]⁺ |
| 141 | Ethyl (Z)-4-(5- bromobenzo[d][1,3]dioxol- 4-yl)-4-hydroxy-2- oxobut-3-enoate PH-LOO-AUK-140 step 2 | 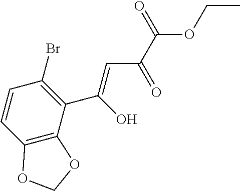 [SMILES: CCOC(=O)C(=O)/C=C(\O)c1c(Br)ccc2c1OCO2] | 343/345 [M + H]⁺ (⁷⁹Br/⁸¹Br) |

### Preparation 20

### Ethyl 1-(4-iodo-2-isopropylphenyl)-5-(5-methoxybenzo[d][1,3]dioxol-4-yl)-1H-pyrazole-3-carboxylate

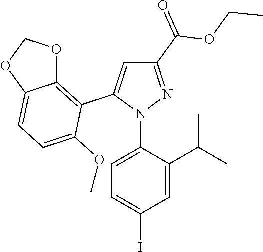 [SMILES: CCOC(=O)c1cc(-c2c(OC)ccc3c2OCO3)n(-c2ccc(I)cc2C(C)C)n1]

A soln of ethyl (Z)-4-hydroxy-4-(5-methoxybenzo[d][1,3]dioxol-4-yl)-2-oxobut-3-enoate (8.12 g, 28.97 mmol) in EtOH (80 mL) was treated with (4-iodo-2-isopropylphenyl)hydrazine (8 g, 28.97 mmol) under N₂ at RT. After stirring under N₂ at 50° C. for 4 h the mixture was concentrated under reduced pressure and purified by silica gel column chromatography PE/EA (2:1) to afford title compound (12 g, 79.6%) as a yellow solid. MS ES+ m/z 535 [M+H]⁺.

The following compounds were prepared in a manner essentially analogous to the preparation method of Ethyl 1-(4-iodo-2-isopropylphenyl)-5-(5-methoxybenzo[d][1,3]dioxol-4-yl)-1H-pyrazole-3-carboxylate using the appropriate reagents, adjusting temperature, adjusting reaction time to determine completion of the reaction, and adjusting the purification system as appropriate.

TABLE 33

| Prep | | | MS ES+ |
|---|---|---|---|
| # | Chemical Name | Structure | m/z |
| 142 | 4-(3-(Ethoxycarbonyl)-5- hydroxy-1H-pyrazol-1- yl)-3-isopropylbenzoic acid | 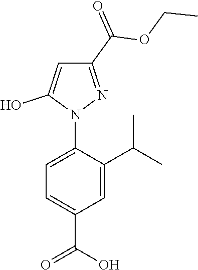 [SMILES: CCOC(=O)c1cc(O)n(-c2ccc(C(=O)O)cc2C(C)C)n1] | 318 [M + H]⁺ |
| 143 | Ethyl 5-(5- bromobenzo[d][1,3] dioxol-4-yl)-1-(4-((3- (dimethylamino)propyl) (methyl)carbamoyl)-2- isopropylphenyl)-1H- pyrazole-3-carboxylate | 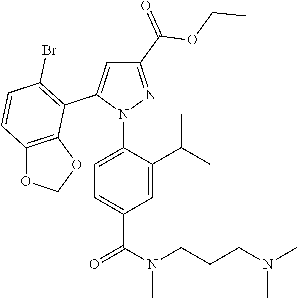 [SMILES: CCOC(=O)c1cc(-c2c(Br)ccc3c2OCO3)n(-c2ccc(C(=O)N(C)CCCN(C)C)cc2C(C)C)n1] | 599/601 [M + H]⁺ (⁷⁹Br/⁸¹Br) |
| 307 | Ethyl 5-(5- bromobenzo[d][1,3] dioxol-4-yl)-1-(4-iodo-2- isopropylphenyl)-1H- pyrazole-3-carboxylate | 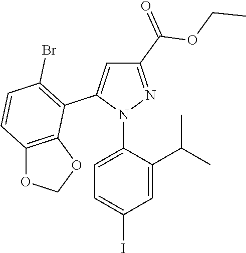 [SMILES: CCOC(=O)c1cc(-c2c(Br)ccc3c2OCO3)n(-c2ccc(I)cc2C(C)C)n1] | 583/585 [M + H]⁺ (⁷⁹Br/⁸¹Br) |

### Preparation 21

### Ethyl 1-(4-bromo-2-isopropylphenyl)-5-(5-methoxybenzo[d][1,3]dioxol-4-yl)-1H-pyrazole-3-carboxylate

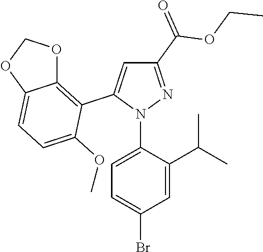 [SMILES: CCOC(=O)c1cc(-c2c(OC)ccc3c2OCO3)n(-c2ccc(Br)cc2C(C)C)n1]

A soln of (4-bromo-2-isopropylphenyl)hydrazine hydrochloride (2500 mg, 9.413 mmol) and sodium (Z)-4-ethoxy-1-(5-methoxybenzo[d][1,3]dioxol-4-yl)-3,4-dioxobut-1-en-1-olate (2.977 g, 9.413 mmol) in AcOH (20 mL) was irradiated with microwave radiation for 45 min at 110° C. The resulting mixture was concentrated, poured into ice-water (30 mL), filtered and the solid washed with H₂O to afford title compound (7.24 g, 9.4 mmol, 99%, 63% Purity) as a brown solid. MS ES+ m/z 487 [M+H]⁺.

The following compounds were prepared in a manner essentially analogous to the preparation method of Ethyl 1-(4-bromo-2-isopropylphenyl)-5-(5-methoxybenzo[d][1,3]dioxol-4-yl)-1H-pyrazole-3-carboxylate using the appropriate reagents, adjusting temperature, adjusting reaction time to determine completion of the reaction, and adjusting the purification system as appropriate.

TABLE 5

| Prep | | | MS ES+ |
|---|---|---|---|
| # | Chemical Name | Structure | m/z |
| 22 | Ethyl 1-(4-bromo-2-(tert- butyl)phenyl)-5-(5- methoxybenzo[d][1,3] dioxol-4-yl)-1H-pyrazole- 3-carboxylate | 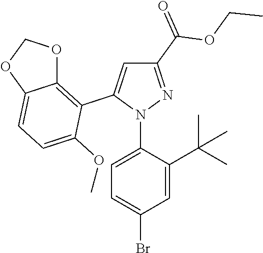 [SMILES: CCOC(=O)c1cc(-c2c(OC)ccc3c2OCO3)n(-c2ccc(Br)cc2C(C)(C)C)n1] | 501 [M + H]⁺ |
| 144 | 1-(6-Chloro-4- isopropylpyridin-3-yl)-5- (5- methoxybenzo[d][1,3] dioxol-4-yl)-1H-pyrazole- 3-carboxylic acid | 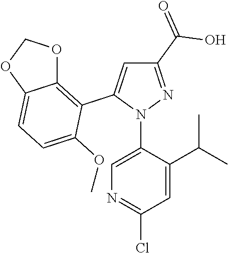 [SMILES: COc1ccc2c(c1-c1cc(C(=O)O)nn1-c1cnc(Cl)cc1C(C)C)OCO2] | 416 [M + H]⁺ |
| 145 | 4-(5-(Benzo[1,2-d:4,5- d′]bis([1,3]dioxole)-4- yl)-3-(ethoxycarbonyl)- 1H-pyrazol-1-yl)-3- isopropylbenzoic acid | 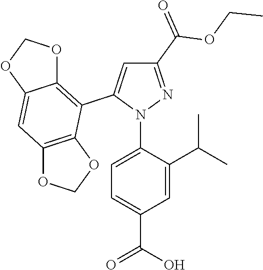 [SMILES: CCOC(=O)c1cc(-c2c3c(cc4c2OCO4)OCO3)n(-c2ccc(C(=O)O)cc2C(C)C)n1] | 467 [M + H]⁺ |

### Preparation 23

### 4-(3-(Ethoxycarbonyl)-5-(5-methoxybenzo[d][1,3]dioxol-4-yl)-1H-pyrazol-1-yl)-3-isopropylbenzoic acid

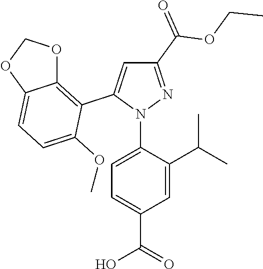 [SMILES: CCOC(=O)c1cc(-c2c(OC)ccc3c2OCO3)n(-c2ccc(C(=O)O)cc2C(C)C)n1]

A soln of 4-hydrazineyl-3-isopropylbenzoic acid (350 mg, 1.80 mmol) and ethyl (Z)-4-hydroxy-4-(5-methoxybenzo[d][1,3]dioxol-4-yl)-2-oxobut-3-enoate (530 mg, 1.80 mmol) in AcOH (6 mL) was stirred at reflux. After 12 h the suspension was poured into an ice-water bath (15 mL), filter, the isolated solid washed with H₂O, and purified by silica gel column chromatography MeOH/DCM (0 to 30%) to afford title compound (390 mg, 48%) as a yellow solid. MS ES+ m/z 453 [M+H]⁺.

### Preparation 146

### 3-Bromo-4-(5-(2,6-dimethoxyphenyl)-3-(ethoxycarbonyl)-1H-pyrazol-1-yl)benzoic acid

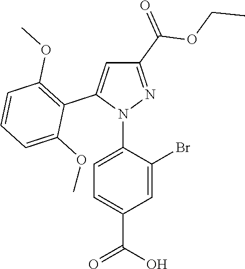 [SMILES: CCOC(=O)c1cc(-c2c(OC)cccc2OC)n(-c2ccc(C(=O)O)cc2Br)n1]

A soln of 3-bromo-4-hydrazinylbenzoic acid (30 g, 90.890 mmol) in EtOH (200 mL) was treated with ethyl (3Z)-4-(2,6-dimethoxyphenyl)-4-hydroxy-2-oxobut-3-enoate (30.57 g, 109.068 mmol). After stirring at RT for 1 h, the precipitated solids were collected by filtration, washed with EtOH (200 mL) (3×50 mL), and dried to afford crude title compound (37 g, 100%) as a light yellow solid. MS ES+ m/z 475/477 [M+H]⁺ (¹⁹Br/⁸¹Br).

### Preparation 147

### Ethyl 1-(4-amino-2-isopropylphenyl)-5-(5-methoxybenzo[d][1,3]dioxol-4-yl)-1H-pyrazole-3-carboxylate

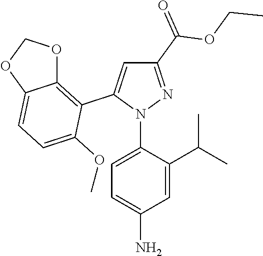 [SMILES: CCOC(=O)c1cc(-c2c(OC)ccc3c2OCO3)n(-c2ccc(N)cc2C(C)C)n1]

A soln of ethyl 1-(4-iodo-2-isopropylphenyl)-5-(5-methoxy-2H-1,3-benzodioxol-4-yl)pyrazole-3-carboxylate (2.5 g, 4.679 mmol) and (2S)-pyrrolidine-2-(13C) carboxylic acid (0.81 g, 7.019 mmol) in DMSO (20 mL) at RT under N₂ was treated with 38% NH₄OH (21.57 g, 233.950 mmol) and CuI (1.78 g, 9.358 mmol). After stirring at 100° C. for 6 h under N₂, the mixture was cooled to room temperature, diluted with EtOAc (150 mL), and washed with brine (5×150 mL). The organic layer was dried over Na₂SO₄, filtered, concentrated and purified by silica gel column chromatography PE/EtOAc (1:1) to afford title compound (980 mg, 49.4%) as a yellow solid. MS ES+ m/z 424 [M+H]⁺.

### Preparation 24

### Ethyl 1-(4-((3-(dimethylamino)propyl)(methyl)carbamoyl)-2-isopropylphenyl)-5-(5-methoxybenzo[d][1,3]dioxol-4-yl)-1H-pyrazole-3-carboxylate

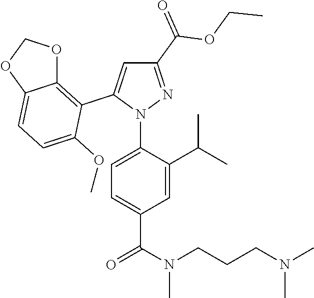 [SMILES: CCOC(=O)c1cc(-c2c(OC)ccc3c2OCO3)n(-c2ccc(C(=O)N(C)CCCN(C)C)cc2C(C)C)n1]

A soln of 4-(3-(ethoxycarbonyl)-5-(5-methoxybenzo[d][1,3]dioxol-4-yl)-1H-pyrazol-1-yl)-3-isopropylbenzoic acid (194 mg, 429 μmol), N,N,N-Trimethyl-1,3-Propanediamine (144 μL, 1.07 mmol), DIEA (554 mg, 738 μL, 10 Eq, 4.29 mmol) and T3P (657 μL, 50% Wt, 1.07 mmol) in degassed DMF (15 mL). After stirring under N₂ at 22° C. for 1.5 h, the mixture was concentrated to afford crude title compound (236 mg, 100%) as a yellow liquid. MS ES+ m/z 551 [M+H]⁺.

The following compounds were prepared in a manner essentially analogous to the preparation method of Ethyl 1-(4-((3-(dimethylamino)propyl)(methyl)carbamoyl)-2-isopropylphenyl)-5-(5-methoxybenzo[d][1,3]dioxol-4-yl)-1H-pyrazole-3-carboxylate using the appropriate reagents (the starting aryl halide is preferably bromo or iodo), adjusting temperature, adjusting reaction time to determine completion of the reaction, and adjusting the purification system as appropriate.

TABLE 6

| Prep | | | MS ES+ |
|---|---|---|---|
| # | Chemical Name | Structure | m/z |
| 148 | Ethyl 1-(4-(2-(4-((tert- butoxycarbonyl)amino) butanoyl)hydrazine-1- carbonyl)-2- isopropylphenyl)-5-(5- methoxybenzo[d][1,3] dioxol-4-yl)-1H-pyrazole- 3-carboxylate | 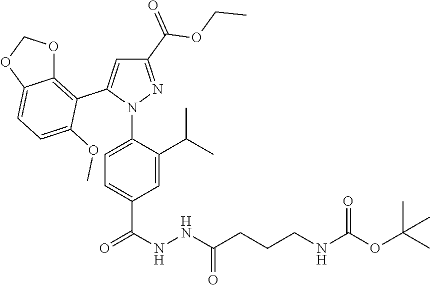 [SMILES: CCOC(=O)c1cc(-c2c(OC)ccc3c2OCO3)n(-c2ccc(C(=O)NNC(=O)CCCNC(=O)OC(C)(C)C)cc2C(C)C)n1] | 650 [M + H]⁺ |
| 149 | Ethyl 1-(4-((4-((tert- butoxycarbonyl)amino) phenyl)carbamoyl)-2- isopropylphenyl)-5-(5- methoxybenzo[d][1,3] dioxol-4-yl)-1H-pyrazole- 3-carboxylate | 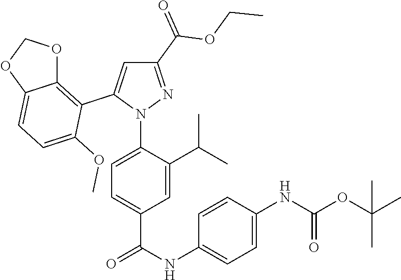 [SMILES: CCOC(=O)c1cc(-c2c(OC)ccc3c2OCO3)n(-c2ccc(C(=O)Nc3ccc(NC(=O)OC(C)(C)C)cc3)cc2C(C)C)n1] | 643 [M + H]⁺ |
| 150 | Ethyl 1-(4-((3-((tert- butoxycarbonyl)amino) phenyl)carbamoyl)-2- isopropylphenyl)-5-(5- methoxybenzo[d][1,3] dioxol-4-yl)-1H-pyrazole- 3-carboxylate | 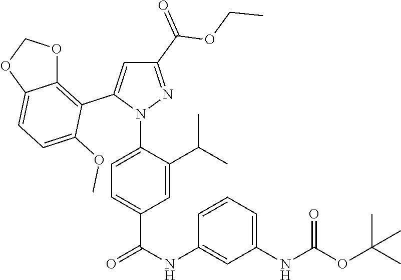 [SMILES: CCOC(=O)c1cc(-c2c(OC)ccc3c2OCO3)n(-c2ccc(C(=O)Nc3cccc(NC(=O)OC(C)(C)C)c3)cc2C(C)C)n1] | 641 [M − H]⁻ |
| 151 | Ethyl 5-(benzo[1,2-d:4,5- d′]bis([1,3]dioxole)-4- yl)-1-(2-isopropyl-4- (methyl(3-(methyl(3- (methylamino)propyl) amino)propyl)carbamoyl) phenyl)-1H-pyrazole-3- carboxylate | 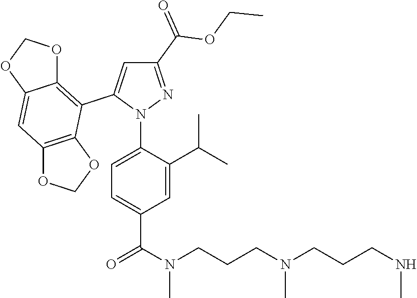 [SMILES: CCOC(=O)c1cc(-c2c3c(cc4c2OCO4)OCO3)n(-c2ccc(C(=O)N(C)CCCN(C)CCCNC)cc2C(C)C)n1] | 622 [M + H]⁺ |
| 308 | Ethyl 1-(4-(2-(4-((tert- butoxycarbonyl)amino) butanoyl)hydrazine-1- carbonyl)-2- isopropylphenyl)-5-(5- methoxybenzo[d][1,3] dioxol-4-yl)-1H-pyrazole- 3-carboxylate | 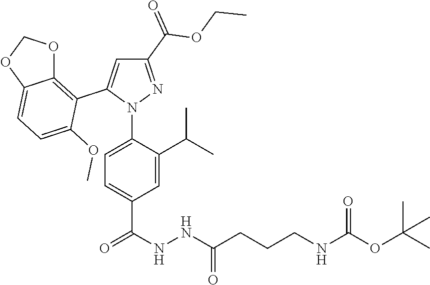 [SMILES: CCOC(=O)c1cc(-c2c(OC)ccc3c2OCO3)n(-c2ccc(C(=O)NNC(=O)CCCNC(=O)OC(C)(C)C)cc2C(C)C)n1] | 552 [M + H]⁺ |

### Preparation 152

### Ethyl 1-(4-((3-(dimethylamino)propyl)(pyridin-2-ylmethyl)carbamoyl)-2-isopropylphenyl)-5-(5-methoxybenzo[d][1,3]dioxol-4-yl)-1H-pyrazole-3-carboxylate

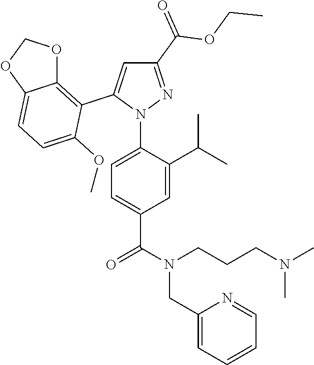 [SMILES: CCOC(=O)c1cc(-c2c(OC)ccc3c2OCO3)n(-c2ccc(C(=O)N(CCCN(C)C)Cc3ccccn3)cc2C(C)C)n1]

A soln of 4-(3-(ethoxycarbonyl)-5-(5-methoxybenzo[d][1,3]dioxol-4-yl)-1H-pyrazol-1-yl)-3-isopropylbenzoic acid (120 mg, 0.265 mmol) in N¹,N¹-dimethyl-N³-(pyridin-2-ylmethyl)propane-1,3-diamine (76.9 mg, 0.398 mmol) in DMF (4 mL) was cooled to 0° C. and treated with DIEA (0.137 mL, 0.796 mmol) and T3P (0.468 mL, 50% Wt, 0.796 mmol). After stirring at RT for 1 h, the mixture was diluted with H₂O (10 mL) and extracted with EtOAc (3×10 mL). The combined organic phases were washed with sat. NaHCO₃soln (10 mL), washed with brine (10 mL), dried over Na₂SO₄, filtered, and concentrated to afford crude title compound (103 mg, 61.9%) as a white solid. MS ES+ m/z 628 [M+H]⁺.

### Preparation 153

### Ethyl 1-(2-bromo-4-((3-(dimethylamino)propyl)(methyl)carbamoyl)phenyl)-5-(2,6-dimethoxyphenyl)-1H-pyrazole-3-carboxylate

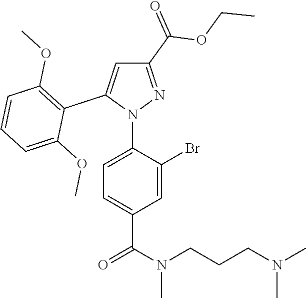 [SMILES: CCOC(=O)c1cc(-c2c(OC)cccc2OC)n(-c2ccc(C(=O)N(C)CCCN(C)C)cc2Br)n1]

A soln of 3-bromo-4-[5-(2,6-dimethoxyphenyl)-3-(ethoxycarbonyl)pyrazol-1-yl]benzoic acid (37 g, 77.846 mmol) and [3-(dimethylamino)propyl](methyl)amine (10.86 g, 93.415 mmol) in ACN (500 mL) at 0° C. was treated with 1-methyl-1H-imidazole (19.17 g, 233.538 mmol) and TCFH (26.21 g, 93.415 mmol). After stirring at room temperature for 1 h, the mixture was diluted with H₂O (800 mL) and extracted with EtOAc (2×800 mL). The combined organic layers were washed with brine (2×500 mL), dried over Na₂SO₄, filtered and purified by reversed-phase flash chromatography with the following conditions: column, C18; mobile phase, 10 to 50% ACN in H₂O (0.1% FA) to afford title compound (27 g, 36.21%) as a light yellow solid. MS ES+ m/z 573/575 [M+H]⁺ (¹⁹Br/⁸¹Br).

### Preparation 154

### Ethyl 1-(4-(5-(3-((tert-butoxycarbonyl)amino)propyl)-1,3,4-thiadiazol-2-yl)-2-isopropylphenyl)-5-(5-methoxybenzo[d][1,3]dioxol-4-yl)-1H-pyrazole-3-carboxylate

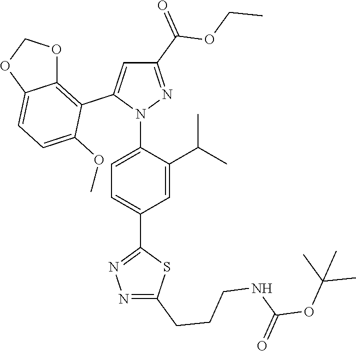 [SMILES: CCOC(=O)c1cc(-c2c(OC)ccc3c2OCO3)n(-c2ccc(-c3nnc(CCCNC(=O)OC(C)(C)C)s3)cc2C(C)C)n1]

A soln of ethyl 1-(4-(2-(4-((tert-butoxycarbonyl)amino)butanoyl)hydrazine-1-carbonyl)-2-isopropylphenyl)-5-(5-methoxybenzo[d][1,3]dioxol-4-yl)-1H-pyrazole-3-carboxylate (400 mg, 0.614 mmol) in anhydrous THF (8 mL), was treated with Lawesson's reagent (496 mg, 1.23 mmol). After microwave heating at 120° C. for 1 h, the mixture was cooled to RT, treated with a sat. soln of NaHCO₃ (10 mL) and extracted with EtOAc (3×25 mL). The combined organic layer was washed with water, brine, dried over Na₂SO₄, concentrated to dryness, and purified by silica gel column chromatography Hep/EtOAc (0-100%) to afford title compound (210 mg, 52.7%) as a white solid. MS ES+ m/z 650 [M+H]⁺.

### Preparation 155

### Ethyl 1-(4-(5-(3-((tert-butoxycarbonyl)amino)propyl)-1,3,4-oxadiazol-2-yl)-2-isopropylphenyl)-5-(5-methoxybenzo[d][1,3]dioxol-4-yl)-1H-pyrazole-3-carboxylate

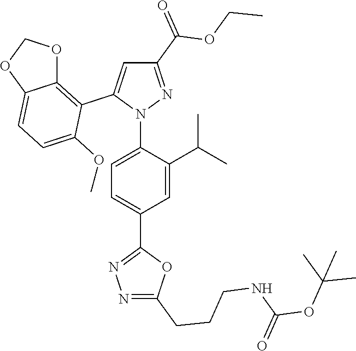 [SMILES: CCOC(=O)c1cc(-c2c(OC)ccc3c2OCO3)n(-c2ccc(-c3nnc(CCCNC(=O)OC(C)(C)C)o3)cc2C(C)C)n1]

A soln of ethyl 1-(4-(2-(4-((tert-butoxycarbonyl)amino)butanoyl)hydrazine-1-carbonyl)-2-isopropylphenyl)-5-(5-methoxybenzo[d][1,3]dioxol-4-yl)-1H-pyrazole-3-carboxylate (260 mg, 0.399 mmol) in anhydrous THF (8 mL), was treated with Burgess reagent (285 mg, 1.20 mmol). After microwave heating at 100° C. for 1 h, the mixture was concentrated and purified by silica gel column chromatography Hep/EtOAc (0-100%) to afford title compound (219 mg, 86.6%) as a yellow gum. MS ES+ m/z 634 [M+H]⁺.

### Preparation 156

### Ethyl 1-(4-(5-(3-aminopropyl)-1,3,4-thiadiazol-2-yl)-2-isopropylphenyl)-5-(5-methoxybenzo[d][1,3]dioxol-4-yl)-1H-pyrazole-3-carboxylate

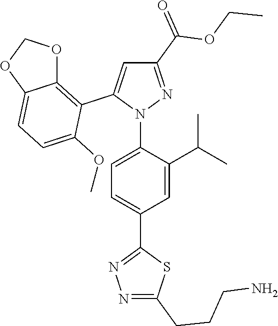 [SMILES: CCOC(=O)c1cc(-c2c(OC)ccc3c2OCO3)n(-c2ccc(-c3nnc(CCCN)s3)cc2C(C)C)n1]

A soln of ethyl 1-(4-(5-(3-((tert-butoxycarbonyl)amino)propyl)-1,3,4-thiadiazol-2-yl)-2-isopropylphenyl)-5-(5-methoxybenzo[d][1,3]dioxol-4-yl)-1H-pyrazole-3-carboxylate (210 mg, 0.323 mmol) in DCM (2 mL) was treated with TFA (2 mL). After stirring at RT for 1 h, the mixture was concentrated, dissolved in DCM (1 mL) and heptane (3 mL), and concentrated to afford crude title compound (191 mg, 89%) as a slight green solid. MS ES+ m/z 550 [M+H]⁺.

The following compounds were prepared in a manner essentially analogous to the preparation method of Ethyl 1-(4-(5-(3-aminopropyl)-1,3,4-thiadiazol-2-yl)-2-isopropylphenyl)-5-(5-methoxybenzo[d][1,3]dioxol-4-yl)-1H-pyrazole-3-carboxylate using the appropriate reagents, adjusting temperature, adjusting reaction time to determine completion of the reaction, and adjusting the purification system as appropriate.

TABLE 34

| | | | MS |
|---|---|---|---|
| Prep | | | ES+ |
| # | Chemical Name | Structure | m/z |
| 157 | Ethyl 1-(4-(5-(3- aminopropyl)-1,3,4- oxadiazol-2-yl)-2- isopropylphenyl)-5-(5- methoxybenzo[d][1,3] dioxol-4-yl)-1H-pyrazole-3- carboxylate | 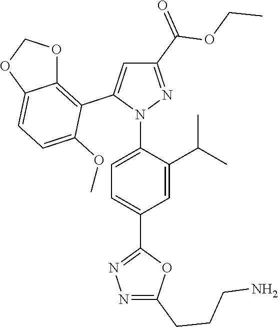 [SMILES: CCOC(=O)c1cc(-c2c(OC)ccc3c2OCO3)n(-c2ccc(-c3nnc(CCCN)o3)cc2C(C)C)n1] | 534 [M + H]⁺ |
| 158 | Ethyl 1-(4-((4- aminophenyl)carbamoyl)- 2-isopropylphenyl)-5-(5- methoxybenzo[d][1,3] dioxol-4-yl)-1H-pyrazole-3- carboxylate | 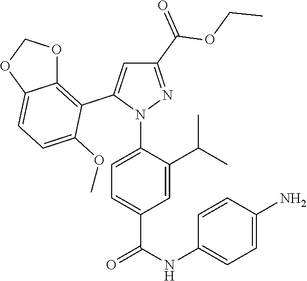 [SMILES: CCOC(=O)c1cc(-c2c(OC)ccc3c2OCO3)n(-c2ccc(C(=O)Nc3ccc(N)cc3)cc2C(C)C)n1] | 543 [M + H]⁺ |
| 159 | Ethyl 1-(4-((3- aminophenyl)carbamoyl)- 2-isopropylphenyl)-5-(5- methoxybenzo[d][1,3] dioxol-4-yl)-1H-pyrazole-3- carboxylate | 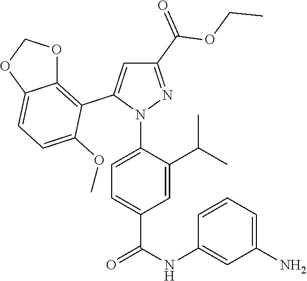 [SMILES: CCOC(=O)c1cc(-c2c(OC)ccc3c2OCO3)n(-c2ccc(C(=O)Nc3cccc(N)c3)cc2C(C)C)n1] | 543 [M + H]⁺ |

### Preparation 160

### 1-(4-(5-(3-(4-(Dimethylamino)butanamido)propyl)-1,3,4-thiadiazol-2-yl)-2-isopropylphenyl)-5-(5-methoxybenzo[d][1,3]dioxol-4-yl)-N-methyl-1H-pyrazole-3-carboxamide

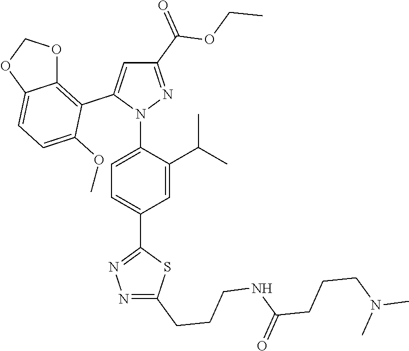 [SMILES: CCOC(=O)c1cc(-c2c(OC)ccc3c2OCO3)n(-c2ccc(-c3nnc(CCCNC(=O)CCCN(C)C)s3)cc2C(C)C)n1]

A soln of ethyl 1-(4-(5-(3-aminopropyl)-1,3,4-thiadiazol-2-yl)-2-isopropylphenyl)-5-(5-methoxybenzo[d][1,3]dioxol-4-yl)-1H-pyrazole-3-carboxylate (120 mg, 0.218 mmol) in DMF (4 mL), was treated with 4-(N,N-Dimethylamino)butanoic acid (28.6 mg, 0.218 mmol). The mixture was cooled to 0° C. and treated with DIEA (0.113 mL, 0.655 mmol) and T3P 50% wt. (0.257 mL, 0.437 mol). After stirring at RT for 1 h, the mixture was diluted with H₂O (15 mL) and extracted with EtOAc (3×10 mL). The combined organic phases were washed with sat. NaHCO₃ solution (10 mL), brine (10 mL), dried over Na₂SO₄, filtered, and concentrated to afford crude title compound (128 mg, 88.5%) as a beige solid. MS ES+ m/z 663 [M+H]⁺.

The following compounds were prepared in a manner essentially analogous to the preparation method of 1-(4-(5-(3-(4-(Dimethylamino)butanamido)propyl)-1,3,4-thiadiazol-2-yl)-2-isopropylphenyl)-5-(5-methoxybenzo[d][1,3]dioxol-4-yl)-N-methyl-1H-pyrazole-3-carboxamide using the appropriate reagents, adjusting temperature, adjusting reaction time to determine completion of the reaction, and adjusting the purification system as appropriate.

TABLE 35

| | | | MS |
|---|---|---|---|
| Prep | | | ES+ |
| # | Chemical Name | Structure | m/z |
| 161 | Ethyl 1-(4-(5-(3-(4- (dimethylamino)butanamido) propyl)-1,3,4- oxadiazol-2-yl)-2- isopropylphenyl)-5-(5- methoxybenzo[d][1,3]dioxol- 4-yl)-1H-pyrazole-3- carboxylate | 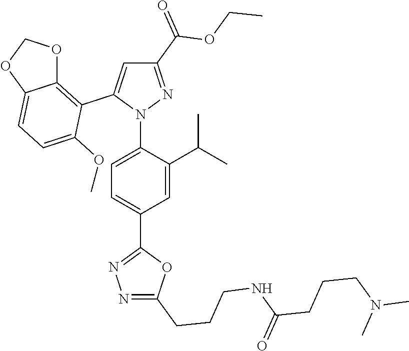 | 447 [M + H]⁺ |
| 162 | Ethyl 1-(4-((4-(4- (dimethylamino)butanamido) phenyl)carbamoyl)- 2-isopropylphenyl)-5-(5- methoxybenzo[d][1,3] dioxol-4-yl)-1H-pyrazole-3- carboxylate | 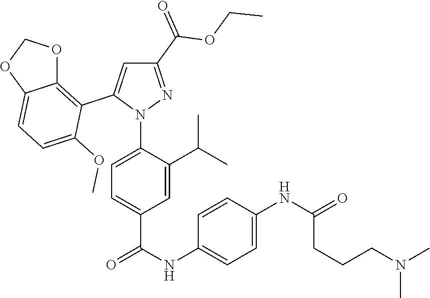 [SMILES: CCOC(=O)c1cc(-c2c(OC)ccc3c2OCO3)n(-c2ccc(C(=O)Nc3ccc(NC(=O)CCCN(C)C)cc3)cc2C(C)C)n1] | 656 [M + H]⁺ |
| 163 | Ethyl 1-(4-((3-(4- (dimethylamino)butanamido) phenyl)carbamoyl)- 2-isopropylphenyl)-5-(5- methoxybenzo[d][1,3] dioxol-4-yl)-1H-pyrazole-3- carboxylate | 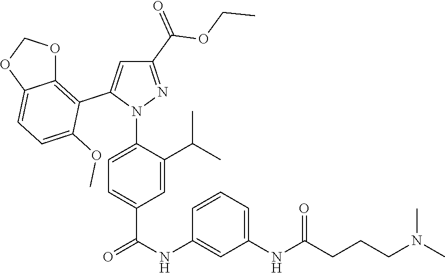 [SMILES: CCOC(=O)c1cc(-c2c(OC)ccc3c2OCO3)n(-c2ccc(C(=O)Nc3cccc(NC(=O)CCCN(C)C)c3)cc2C(C)C)n1] | 656 [M + H]⁺ |

### Preparation 164

### Ethyl 1-(4-(4-(dimethylamino)butanamido)-2-isopropylphenyl)-5-(5-methoxybenzo[d][1,3]dioxol-4-yl)-1H-pyrazole-3-carboxylate

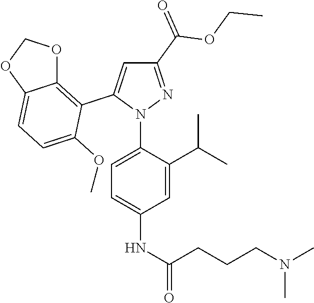 [SMILES: CCOC(=O)c1cc(-c2c(OC)ccc3c2OCO3)n(-c2ccc(NC(=O)CCCN(C)C)cc2C(C)C)n1]

A soln of ethyl 1-(4-amino-2-isopropylphenyl)-5-(5-methoxy-2H-1,3-benzodioxol-4-yl)pyrazole-3-carboxylate (200 mg, 0.472 mmol) and 4-(dimethylamino)butanoic acid hydrochloride (119 mg, 0.708 mmol) in DCM (5 mL) at RT under N₂ was treated with DIEA (305 mg, 2.360 mmol) and T3P (50% in EtOAc) (451 mg, 1.416 mmol). After stirring for 4 h, the mixture was concentrated purified by reversed-phase flash chromatography with the following conditions: column, C18; mobile phase, 30 to 50% ACN in H₂O (0.1% FA) to afford title compound (200 mg, 78.9%) as a white solid. MS ES+ m/z 537 [M+H]⁺.

### Preparation 25

### 1-(4-Iodo-2-isopropylphenyl)-5-(5-methoxybenzo[d][1,3]dioxol-4-yl)-1H-pyrazole-3-carboxylic acid

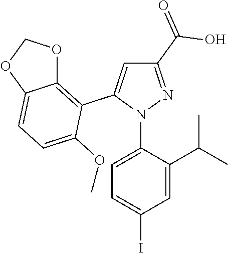 [SMILES: COc1ccc2c(c1-c1cc(C(=O)O)nn1-c1ccc(I)cc1C(C)C)OCO2]

A soln of ethyl 1-(4-iodo-2-isopropylphenyl)-5-(5-methoxybenzo[d][1,3]dioxol-4-yl)-1H-pyrazole-3-carboxylate (6 g, 11.22 mmol) in MeOH (60 mL) and H₂O (12 mL) was treated with LiOH·H₂O (0.94 g, 22.45 mmol) in portions under N₂ at RT. After stirring under N₂ at 50° C. for 4 h the mixture was diluted with H₂O (100 mL) and extracted with EA (2×200 mL). The combined organic layers were washed with brine (2×100 mL), dried over anhydrous Na₂SO₄. The filtrate was concentrated under reduced pressure and purified by silica gel column chromatography PE/EtOAc (2:1) to afford title compound (5.4 g, 94.9%) as a light yellow solid. MS ES+ m/z 507 [M+H]⁺.

The following compounds were prepared in a manner essentially analogous to the preparation method of 1-(4-Iodo-2-isopropylphenyl)-5-(5-methoxybenzo[d][1,3]dioxol-4-yl)-1H-pyrazole-3-carboxylic acid using the appropriate reagents, adjusting temperature, adjusting reaction time to determine completion of the reaction, and adjusting the purification system as appropriate.

TABLE 6

| | | | MS |
|---|---|---|---|
| Prep | | | ES+ |
| # | Chemical Name | Structure | m/z |
| 26 | 1-(4-Bromo-2- isopropylphenyl)-5-(5- methoxybenzo[d][1,3] dioxol-4-yl)-1H-pyrazole-3- carboxylic acid | 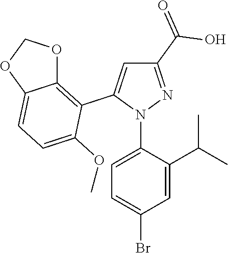 [SMILES: COc1ccc2c(c1-c1cc(C(=O)O)nn1-c1ccc(Br)cc1C(C)C)OCO2] | 459 [M + H]⁺ |
| 27 | 1-(4-((3- (Dimethylamino)propyl) (methyl)carbamoyl)-2- isopropylphenyl)-5-(5- methoxybenzo[d][1,3] dioxol-4-yl)-1H-pyrazole-3- carboxylic acid | 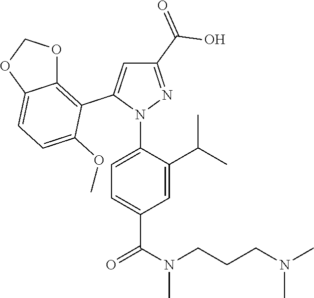 [SMILES: COc1ccc2c(c1-c1cc(C(=O)O)nn1-c1ccc(C(=O)N(C)CCCN(C)C)cc1C(C)C)OCO2] | 523 [M + H]⁺ |
| 28 | 1-(4-Bromo-2-(tert- butyl)phenyl)-5-(5- methoxybenzo[d][1,3] dioxol-4-yl)-1H-pyrazole-3- carboxylic acid | 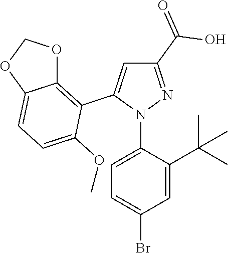 [SMILES: COc1ccc2c(c1-c1cc(C(=O)O)nn1-c1ccc(Br)cc1C(C)(C)C)OCO2] | 473 [M + H]⁺ |
| 165 | 1-(4-(5-(3-(4- (Dimethylamino) butanamido) propyl)-1,3,4- thiadiazol-2-yl)-2- isopropylphenyl)-5-(5- methoxybenzo[d][1,3] dioxol-4-yl)-1H-pyrazole-3- carboxylic acid | 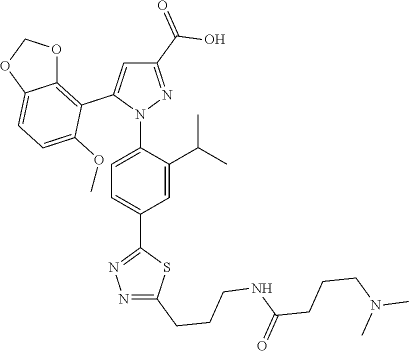 [SMILES: COc1ccc2c(c1-c1cc(C(=O)O)nn1-c1ccc(-c3nnc(CCCNC(=O)CCCN(C)C)s3)cc1C(C)C)OCO2] | 635 [M + H]⁺ |
| 166 | 1-(4-(5-(3-(4- (Dimethylamino) butanamido) propyl)-1,3,4- oxadiazol-2-yl)-2- isopropylphenyl)-5-(5- methoxybenzo[d][1,3] dioxol-4-yl)-1H-pyrazole-3- carboxylic acid | 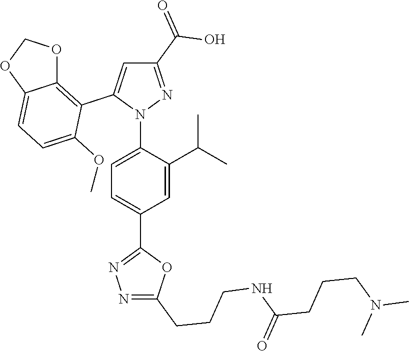 [SMILES: COc1ccc2c(c1-c1cc(C(=O)O)nn1-c1ccc(-c3nnc(CCCNC(=O)CCCN(C)C)o3)cc1C(C)C)OCO2] | 619 [M + H]⁺ |
| 167 | 1-(4-((3- (Dimethylamino)propyl) (pyridin-2- ylmethyl)carbamoyl)-2- isopropylphenyl)-5-(5- methoxybenzo[d][1,3] dioxol-4-yl)-1H-pyrazole-3- carboxylic acid | 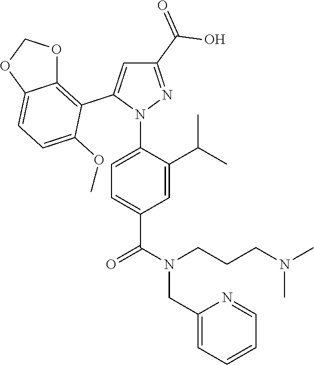 [SMILES: COc1ccc2c(c1-c1cc(C(=O)O)nn1-c1ccc(C(=O)N(CCCN(C)C)Cc3ccccn3)cc1C(C)C)OCO2] | 600 [M + H]⁺ |
| 168 | 1-(4-((4-(4- (Dimethylamino) butanamido) phenyl)carbamoyl)- 2-isopropylphenyl)-5-(5- methoxybenzo[d][1,3] dioxol-4-yl)-1H-pyrazole-3- carboxylic acid | 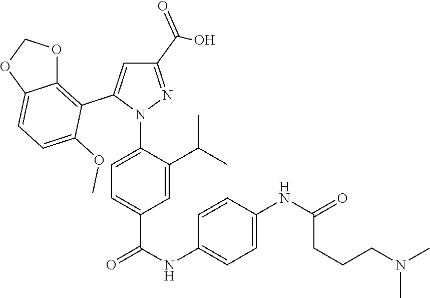 [SMILES: COc1ccc2c(c1-c1cc(C(=O)O)nn1-c1ccc(C(=O)Nc3ccc(NC(=O)CCCN(C)C)cc3)cc1C(C)C)OCO2] | 628 [M + H]⁺ |
| 169 | 1-(4-((3-(4- (Dimethylamino) butanamido) phenyl)carbamoyl)- 2-isopropylphenyl)-5-(5- methoxybenzo[d][1,3] dioxol-4-yl)-1H-pyrazole-3- carboxylic acid | 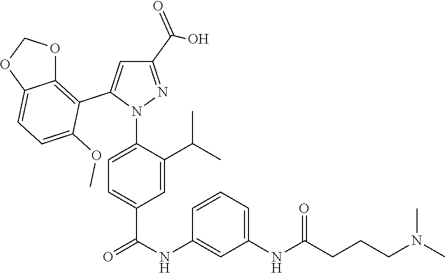 [SMILES: COc1ccc2c(c1-c1cc(C(=O)O)nn1-c1ccc(C(=O)Nc3cccc(NC(=O)CCCN(C)C)c3)cc1C(C)C)OCO2] | 628 [M + H]⁺ |
| 170 | 1-(2-Bromo-4-((3- (dimethylamino)propyl) (methyl)carbamoyl) phenyl)-5-(2,6- dimethoxyphenyl)-1H- pyrazole-3-carboxylic acid | 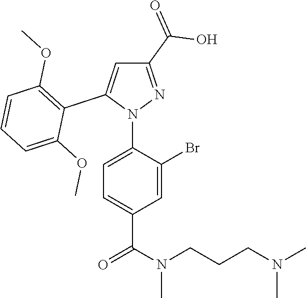 [SMILES: COc1cccc(OC)c1-c1cc(C(=O)O)nn1-c1ccc(C(=O)N(C)CCCN(C)C)cc1Br] | 545/547 [M + H]⁺ (⁷⁹Br/ ⁸¹Br) |
| 171 | 5-(5- Bromobenzo[d][1,3]dioxol- 4-yl)-1-(4-((3- (dimethylamino)propyl) (methyl)carbamoyl)-2- isopropylphenyl)-1H- pyrazole-3-carboxylic acid | 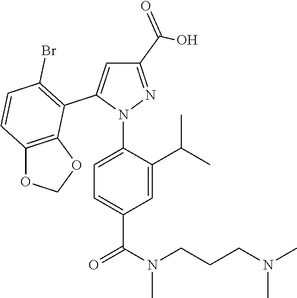 [SMILES: CC(C)c1cc(C(=O)N(C)CCCN(C)C)ccc1-n1nc(C(=O)O)cc1-c1c(Br)ccc2c1OCO2] | 571/573 [M + H]⁺ (⁷⁹Br/ ⁸¹Br) |
| 172 | 1-(4-(4- (Dimethylamino) butanamido)-2- isopropylphenyl)-5-(5- methoxybenzo[d][1,3] dioxol-4-yl)-1H-pyrazole-3- carboxylic acid | 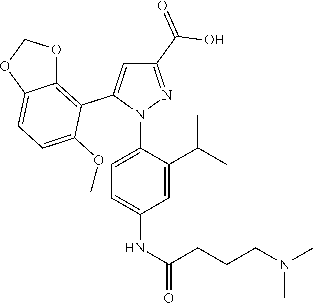 [SMILES: COc1ccc2c(c1-c1cc(C(=O)O)nn1-c1ccc(NC(=O)CCCN(C)C)cc1C(C)C)OCO2] | 509 [M + H]⁺ |
| 309 | 5-(5- bromobenzo[d][1,3]dioxol- 4-yl)-1-(4-hydroxy-2- isopropylphenyl)-1H- pyrazole-3-carboxylic acid | 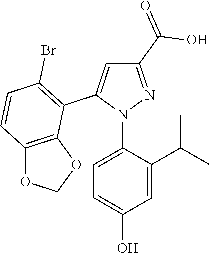 [SMILES: CC(C)c1cc(O)ccc1-n1nc(C(=O)O)cc1-c1c(Br)ccc2c1OCO2] | 445/447 [M + H]⁺ (⁷⁹Br/ ⁸¹Br) |

### Preparation 173

### Ethyl 5-bromo-1-(4-(ethoxycarbonyl)-2-isopropylphenyl)-1H-pyrazole-3-carboxylate

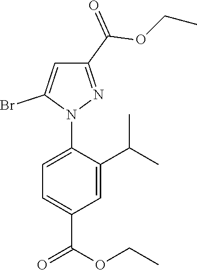 [SMILES: CCOC(=O)c1ccc(-n2nc(C(=O)OCC)cc2Br)c(C(C)C)c1]

4-[3-(Ethoxycarbonyl)-5-hydroxypyrazol-1-yl]-3-isopropylbenzoic acid (1 g, 3.14 mmol, 1.00 equiv) was treated with phosphoroyl tribromide (20 g) at RT under N₂. After stirring at 150° C. ON under N₂, the mixture was allowed to cool to RT, and diluted with EtOAc (800 mL) prior to treatment with EtOH (1.6 L) at 0° C. The resulting mixture was filtered, the filter cake washed with EtOAc (3×100 mL), the filtrate concentrated and purified by silica gel column chromatography PE/EtOAc (7:1) to afford title compound (24 g, 30%) as a yellow oil MS ES+ m/z 409/411 [M+H]⁺ (¹⁹Br/⁸¹Br).

### Preparation 174

### 5-Bromo-1-(4-(ethoxycarbonyl)-2-isopropylphenyl)-1H-pyrazole-3-carboxylic acid

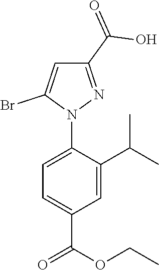 [SMILES: CCOC(=O)c1ccc(-n2nc(C(=O)O)cc2Br)c(C(C)C)c1]

A soln of ethyl 5-bromo-1-[4-(ethoxycarbonyl)-2-isopropylphenyl]pyrazole-3-carboxylate (23 g, 56.19 mmol) in DCE (400 mL) was treated with trimethylstannanol (20.32 g, 112.39 mmol). After stirring at 80° C. for 2 h under N₂, the mixture was to cooled to RT, diluted with H₂O (1000 mL), extracted with EtOAc (3×600 mL), washed with brine (1×800 mL), dried over anhydrous Na₂SO₄, filtered and purified by silica gel column chromatography PE/EtOAc (1:1) to afford title compound (9.5 g, 44.3%) as a yellow solid. MS ES+ m/z 381/383 [M+H]⁺ (¹⁹Br/⁸¹Br).

### Preparation 29

### tert-Butyl (5r,7r)-2-(1-(4-iodo-2-isopropylphenyl)-5-(5-methoxybenzo[d][1,3]dioxol-4-yl)-1H-pyrazole-3-carboxamido)adamantane-2-carboxylate

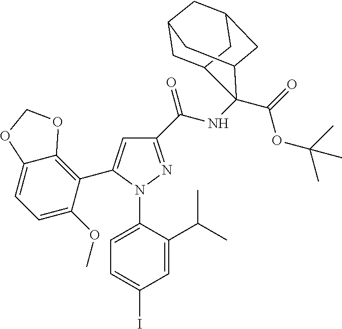 [SMILES: COc1ccc2c(c1-c1cc(C(=O)NC3(C(=O)OC(C)(C)C)C4CC5CC(C4)CC3C5)nn1-c1ccc(I)cc1C(C)C)OCO2]

A soln of 1-(4-iodo-2-isopropylphenyl)-5-(5-methoxybenzo[d][1,3]dioxol-4-yl)-1H-pyrazole-3-carboxylic acid (5 g, 9.876 mmol) and tert-butyl 2-aminoadamantane-2-carboxylate (3.72 g, 14.81 mmol) in ACN (50 mL) was treated with 1-methyl-1H-imidazole (2.43 g, 29.628 mmol) and TCFH (4.16 g, 14.81 mmol). After stirring under N₂ at RT for 2 h the mixture was diluted with water (200 mL) and extracted with EA (2×300 mL). The combined organic layers were washed with brine (2×100 mL), dried over anhydrous Na₂SO₄, and filtered. The filtrate was concentrated under reduced pressure and lyophilized under vacuum to afford title compound (5.2 g, 71.1%) as a yellow solid. MS ES− m/z 738 [M−H]⁻.

The following compounds were prepared in a manner essentially analogous to the preparation method of tert-Butyl (5r,7r)-2-(1-(4-iodo-2-isopropylphenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 36

| Prep | | | MS ES+ |
|---|---|---|---|
| # | Chemical Name | Structure | m/z |
| 175 | tert-Butyl (5r,7r)-2-(5- bromo-1-(4- (ethoxycarbonyl)-2- isopropylphenyl)-1H- pyrazole-3- carboxamido)adamantane- 2-carboxylate | 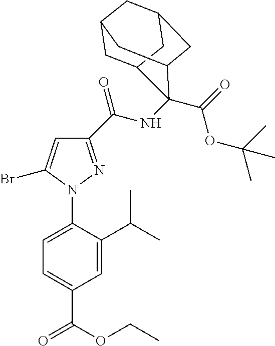 [SMILES: CCOC(=O)c1ccc(-n2nc(C(=O)NC3(C(=O)OC(C)(C)C)C4CC5CC(C4)CC3C5)cc2Br)c(C(C)C)c1] | 614/616 [M + H]⁺ (⁷⁹Br/⁸¹Br) |
| 176 | tert-Butyl (5r,7r)-2-(5-(5- bromobenzo[d][1,3] dioxol-4-yl)-1-(4-((3- (dimethylamino)propyl) (methyl)carbamoyl)-2- isopropylphenyl)-1H- pyrazole-3- carboxamido)adamantane- 2-carboxylate | 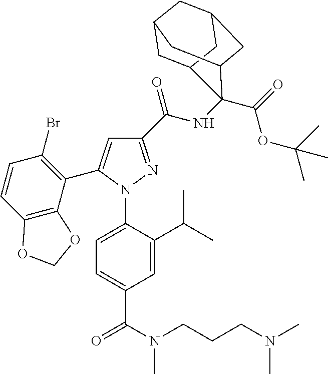 [SMILES: CC(C)c1cc(C(=O)N(C)CCCN(C)C)ccc1-n1nc(C(=O)NC2(C(=O)OC(C)(C)C)C3CC4CC(C3)CC2C4)cc1-c1c(Br)ccc2c1OCO2] | 804/806 [M + H]⁺ (⁷⁹Br/⁸¹Br) |
| 177 | N-((5r,7r)-2-(1-Benzyl- 1H-tetrazol-5- yl)adamantan-2-yl)-1-(4- (4- (dimethylamino) butanamido)- 2-isopropylphenyl)- 5-(5- methoxybenzo[d][1,3] dioxol-4-yl)-1H-pyrazole-3- carboxamide | 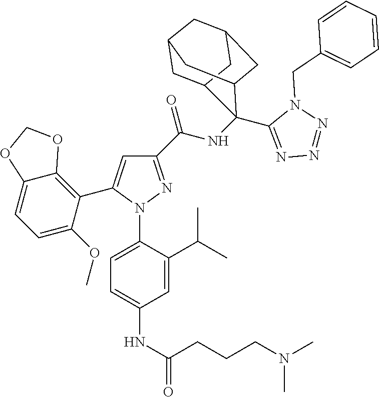 [SMILES: COc1ccc2c(c1-c1cc(C(=O)NC3(c4nnnn4Cc4ccccc4)C4CC5CC(C4)CC3C5)nn1-c1ccc(NC(=O)CCCN(C)C)cc1C(C)C)OCO2] | 801 [M + H]⁺ |

### Preparation 389

### tert-Butyl (5r,7r)-2-(5-(5-bromobenzo[d][1,3]dioxol-4-yl)-1-(4-hydroxy-2-isopropylphenyl)-1H-pyrazole-3-carboxamido)adamantane-2-carboxylate

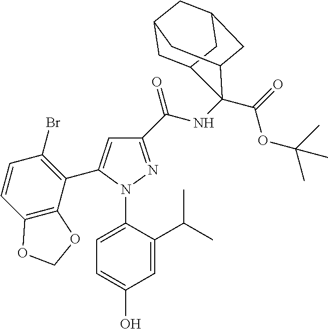 [SMILES: CC(C)c1cc(O)ccc1-n1nc(C(=O)NC2(C(=O)OC(C)(C)C)C3CC4CC(C3)CC2C4)cc1-c1c(Br)ccc2c1OCO2]

A soln of tert-butyl 2-aminoadamantane-2-carboxylate (677 mg, 2.7 mmol) in THF (10 mL) at RT under N₂ was treated with 5-(5-bromo-2H-1,3-benzodioxol-4-yl)-1-(4-hydroxy-2-isopropylphenyl)pyrazole-3-carboxylic acid (1.2 g, 2.7 mmol), EDCI·HCl (619 mg, 3.2 mmol), HOBT (437 mg, 3.2 mmol), DIEA (696 mg, 5.4 mmol). The mixture was stirred ON, concentrated under reduced pressure, and purified by reversed-phase flash chromatography with the following conditions: column, C18; mobile phase, 10% to 50% ACN in H₂O (0.1% FA) to afford title compound (1 g, 55%) as a white solid. MS ES+ m/z 678/680 [M+H]⁺ (¹⁹Br/⁸¹Br).

### Preparation 178

### tert-Butyl (5r,7r)-2-(5-(5-cyanobenzo[d][1,3]dioxol-4-yl)-1-(4-((3-(dimethylamino)propyl)(methyl)carbamoyl)-2-isopropylphenyl)-1H-pyrazole-3-carboxamido)adamantane-2-carboxylate

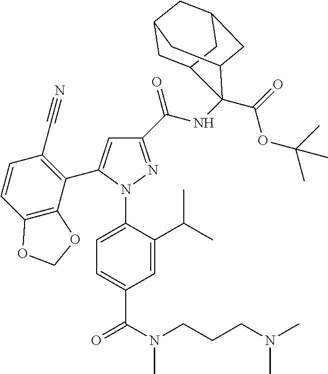 [SMILES: CC(C)c1cc(C(=O)N(C)CCCN(C)C)ccc1-n1nc(C(=O)NC2(C(=O)OC(C)(C)C)C3CC4CC(C3)CC2C4)cc1-c1c(C#N)ccc2c1OCO2]

A soln of tert-butyl 2-[5-(5-bromo-2H-1,3-benzodioxol-4-yl)-1-(4-{[3-(dimethylamino)propyl](methyl)carbamoyl}-2-isopropylphenyl)pyrazole-3-amido]adamantane-2-carboxylate (300 mg, 0.373 mmol) and Zn(CN)2 (88 mg, 0.746 mmol) in DMF (2 mL) was treated with XantPhos Pd G3 (71 mg, 0.075 mmol) at RT under N₂. After stirring at 80° C. for 2 h under N₂, the mixture was concentrated and purified by reversed-phase flash chromatography with the following conditions: column, C18; mobile phase, 30% to 70% ACN in H₂O (0.1% FA) to afford title compound (240 mg, 85.74%) as a light yellow solid. MS ES+ m/z 752 [M+H]⁺.

The following compounds were prepared in a manner essentially analogous to the preparation method of tert-Butyl (5r,7r)-2-(5-(5-cyanobenzo[d][1,3]dioxol-4-yl)-1-(4-((3-(dimethylamino)propyl)(methyl)carbamoyl)-2-isopropylphenyl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 49

| Prep | | | MS ES+ |
|---|---|---|---|
| # | Chemical Name | Structure | m/z |
| 310 | tert-Butyl (5r,7r)-2-(5-(5- cyanobenzo[d][1,3]dioxol- 4-yl)-1-(4-hydroxy-2- isopropylphenyl)-1H- pyrazole-3- carboxamido)adamantane- 2-carboxylatetert-butyl (5r,7r)-2-(5-(5- cyanobenzo[d][1,3]dioxol- 4-yl)-1-(4-hydroxy-2- isopropylphenyl)-1H- pyrazole-3- carboxamido)adamantane- 2-carboxylate | 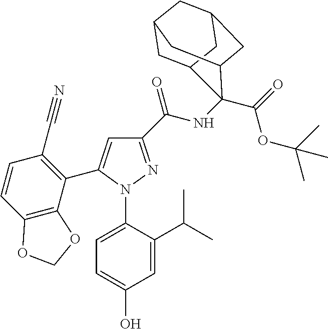 [SMILES: CC(C)c1cc(O)ccc1-n1nc(C(=O)NC2(C(=O)OC(C)(C)C)C3CC4CC(C3)CC2C4)cc1-c1c(C#N)ccc2c1OCO2] | 625 [M + H]⁺ |

### Preparation 179

### tert-Butyl (5r,7r)-2-(1-(2-bromo-4-((3-(dimethylamino)propyl)(methyl)carbamoyl)phenyl)-5-(2,6-dimethoxyphenyl)-1H-pyrazole-3-carboxamido)adamantane-2-carboxylate

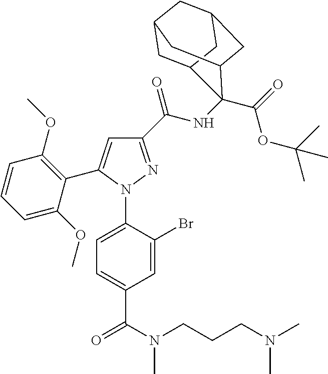 [SMILES: COc1cccc(OC)c1-c1cc(C(=O)NC2(C(=O)OC(C)(C)C)C3CC4CC(C3)CC2C4)nn1-c1ccc(C(=O)N(C)CCCN(C)C)cc1Br]

A soln of 1-(2-bromo-4-{[3-(dimethylamino)propyl](methyl)carbamoyl}phenyl)-5-(2,6-dimethoxyphenyl)pyrazole-3-carboxylic acid (16 g, 29.334 mmol) in DMF (150 mL) was treated with PyBOP (22.90 g, 44.001 mmol) and DIEA (11.37 g, 88.002 mmol). After stirring at RT for 10 min, the mixture was treated with tert-butyl 2-aminoadamantane-2-carboxylate (14.75 g, 58.668 mmol). After stirring at RT for 2 h, the mixture was diluted with H₂O (800 mL) and extracted with EtOAc (2×500 mL). The combined organic layers were dried over Na₂SO₄, filtered, concentrated and purified by reversed-phase flash chromatography with the following conditions: column, C18; mobile phase, 10 to 50% ACN in H₂O (0.1% FA) to afford title compound (7.5 g, 32.83%) as a light yellow solid. MS ES+ m/z 778/780 [M+H]⁺ (79Br/⁸¹Br).

### Preparation 180

### 4-(5-Bromo-3-(((5r,7r)-2-(tert-butoxycarbonyl)adamantan-2-yl)carbamoyl)-1H-pyrazol-1-yl)-3-isopropylbenzoic acid

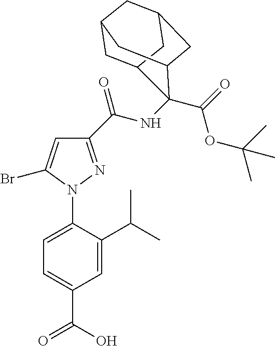 [SMILES: CC(C)c1cc(C(=O)O)ccc1-n1nc(C(=O)NC2(C(=O)OC(C)(C)C)C3CC4CC(C3)CC2C4)cc1Br]

A soln of tert-butyl 2-{5-bromo-1-[4-(ethoxycarbonyl)-2-isopropylphenyl]pyrazole-3-amido}adamantane-2-carboxylate (3.60 g, 5.85 mmol) in THF (50 mL) and H₂O (10 mL) was treated with LiOH·H₂O (368.68 mg, 8.78 mmol) at RT under N₂. After stirring at 50° C. for 4 h under N₂, the mixture was cooled to RT, concentrated, diluted with H₂O (800 mL), and extracted with EtOAc (2×400 mL). The combined organic layers were washed with brine (1×300 mL), dried over anhydrous Na₂SO₄, filtered, and concentrated under reduced pressure. The residue was purified by reversed-phase flash chromatography with the following conditions: column, C18; mobile phase, 70 to 90% ACN in H₂O (0.1% FA) to afford title compound (2.85 g, 82.9%) as a white solid. MS ES+ m/z 586/588 [M+H]⁺ (¹⁹Br/⁸¹Br).

The following compounds were prepared in a manner essentially analogous to the preparation method of 4-(5-Bromo-3-(((5r,7r)-2-(tert-butoxycarbonyl)adamantan-2-yl)carbamoyl)-1H-pyrazol-1-yl)-3-isopropylbenzoic acid using the appropriate reagents, adjusting temperature, adjusting reaction time to determine completion of the reaction, and adjusting the purification system as appropriate.

TABLE 50

| Prep | | | MS ES+ |
|---|---|---|---|
| # | Chemical Name | Structure | m/z |
| 311 | 5-Borono-3- fluoropicolinic acid | 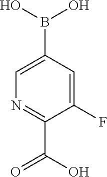 [SMILES: O=C(O)c1ncc(B(O)O)cc1F] | 186 [M + H]⁺ |

### Preparation 181

### tert-Butyl (5r,7r)-2-(1-(4-amino-2-isopropylphenyl)-5-bromo-1H-pyrazole-3-carboxamido)adamantane-2-carboxylate

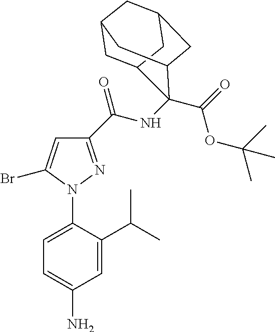 [SMILES: CC(C)c1cc(N)ccc1-n1nc(C(=O)NC2(C(=O)OC(C)(C)C)C3CC4CC(C3)CC2C4)cc1Br]

A soln of 4-(5-bromo-3-{[2-(tert-butoxycarbonyl)adamantan-2-yl]carbamoyl}pyrazol-1-yl)-3-isopropylbenzoic acid (4.00 g, 6.82 mmol) and azidotrimethylsilane (3.14 g, 27.28 mmol) in Dioxane (60 mL) was treated with T3P (50% in EtOAc) (8.68 g, 13.64 mmol) and Et₃N (2.76 g, 27.28 mmol) at RT under N₂. After stirring at 100° C. for 2 h the mixture was treated with H₂O (1.23 g, 68.2 mmol) at RT. After stirring at 100° C. for 2 h the mixture was cooled to RT, the mixture was diluted with H₂O (500 mL) and extracted with EtOAc (3×300 mL). The combined organic layers were washed with brine (1×500 mL), dried over anhydrous Na₂SO₄, filtered, and concentrated. The residue was purified by reversed-phase flash chromatography with the following conditions: column, C18; mobile phase, 70 to 90% ACN in H₂O (10 mmol/L NH₄HCO₃) to afford title compound (1.9 g, 50%) as a light yellow solid. MS ES+ m/z 557/559 [M+H]⁺ (¹⁹Br/⁸¹Br).

### Preparation 30

### tert-Butyl (5r,7r)-2-(1-(4-bromo-2-isopropylphenyl)-5-(5-methoxybenzo[d][1,3]dioxol-4-yl)-1H-pyrazole-3-carboxamido)adamantane-2-carboxylate

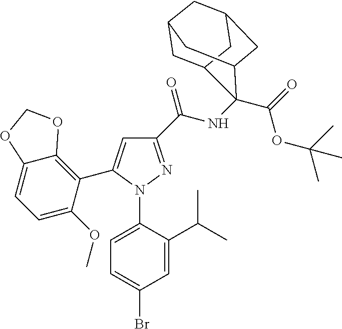 [SMILES: COc1ccc2c(c1-c1cc(C(=O)NC3(C(=O)OC(C)(C)C)C4CC5CC(C4)CC3C5)nn1-c1ccc(Br)cc1C(C)C)OCO2]

A soln of 1-(4-Bromo-2-isopropylphenyl)-5-(5-methoxybenzo[d][1,3]dioxol-4-yl)-1H-pyrazole-3-carboxylic acid (3000 mg, 6.532 mmol) in DMF (12 mL) at 0° C. was treated with T3P (5.768 mL, 19.60 mmol), DIEA (3.37 mL, 19.60 mmol), and tert-butyl (5r,7r)-2-aminoadamantane-2-carboxylate (3.284 g, 13.06 mmol). After stirring at RT for 24 h the mixture was treated with another batch of tert-butyl (5r,7r)-2-aminoadamantane-2-carboxylate (3.284 g, 13.06 mmol), T3P (5.768 mL, 19.60 mmol), DIEA (3.37 mL, 19.60 mmol) and stirred for 24 h. The mixture was cooled to 0° C. and treated with cold ice H₂O. The resultant mixture was filtered, the isolated solid washed with H₂O (5 mL) and then dried in a heated oven ON to afford title compound (4.0 g, 5.8 mmol, 88%) as a beige solid. MS ES+ m/z 692 [M+H]⁺.

The following compounds were prepared in a manner essentially analogous to the preparation method of tert-Butyl (5r,7r)-2-(1-(4-bromo-2-isopropylphenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 7

| Prep | | | MS ES+ |
|---|---|---|---|
| # | Chemical Name | Structure | m/z |
| 31 | tert-Butyl (5r,7r)-2-(1-(4- bromo-2-(tert- butyl)phenyl)-5-(5- methoxybenzo[d][1,3] dioxol-4-yl)-1H-pyrazole-3- carboxamido)adamantane- 2-carboxylate | 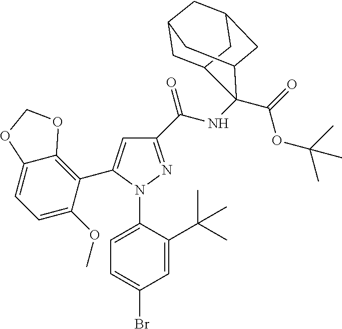 [SMILES: COc1ccc2c(c1-c1cc(C(=O)NC3(C(=O)OC(C)(C)C)C4CC5CC(C4)CC3C5)nn1-c1ccc(Br)cc1C(C)(C)C)OCO2] | 706/708 [M + H]⁺ (⁷⁹Br/⁸¹Br) |
| 182 | tert-Butyl (5r,7r)-2-(1-(4- (5-(3-(4- (dimethylamino) butanamido) propyl)-1,3,4- thiadiazol-2-yl)-2- isopropylphenyl)-5-(5- methoxybenzo[d][1,3] dioxol-4-yl)-1H-pyrazole-3- carboxamido)adamantane- 2-carboxylate | 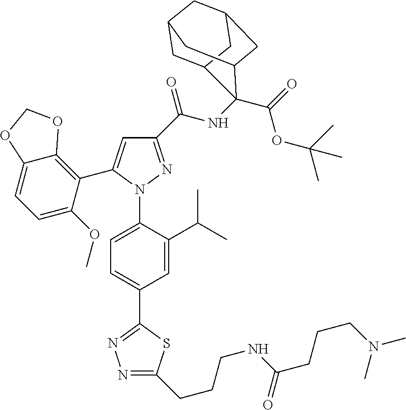 [SMILES: COc1ccc2c(c1-c1cc(C(=O)NC3(C(=O)OC(C)(C)C)C4CC5CC(C4)CC3C5)nn1-c1ccc(-c3nnc(CCCNC(=O)CCCN(C)C)s3)cc1C(C)C)OCO2] | 869 [M + H]⁺ |
| 183 | tert-Butyl (5r,7r)-2-(1-(4- (5-(3-(4- (dimethylamino) butanamido)propyl)-1,3,4- oxadiazol-2-yl)-2- isopropylphenyl)-5-(5- methoxybenzo[d][1,3] dioxol-4-yl)-1H-pyrazole-3- carboxamido)adamantane- 2-carboxylate | 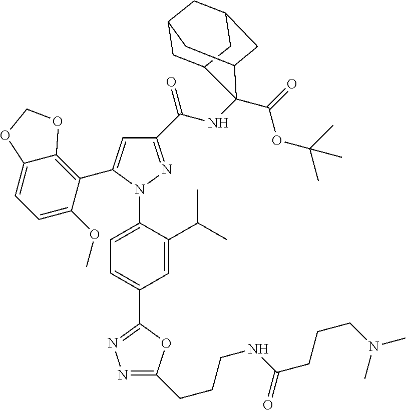 [SMILES: COc1ccc2c(c1-c1cc(C(=O)NC3(C(=O)OC(C)(C)C)C4CC5CC(C4)CC3C5)nn1-c1ccc(-c3nnc(CCCNC(=O)CCCN(C)C)o3)cc1C(C)C)OCO2] | 853 [M + H]⁺ |
| 184 | tert-Butyl (5r,7r)-2-(1-(4- ((3- (dimethylamino)propyl) (pyridin-2- ylmethyl)carbamoyl)-2- isopropylphenyl)-5-(5- methoxybenzo[d][1,3] dioxol-4-yl)-1H-pyrazole-3- carboxamido)adamantane- 2-carboxylate | 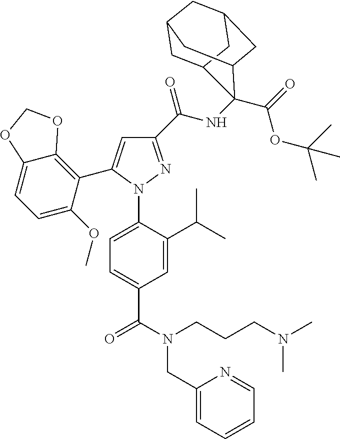 [SMILES: COc1ccc2c(c1-c1cc(C(=O)NC3(C(=O)OC(C)(C)C)C4CC5CC(C4)CC3C5)nn1-c1ccc(C(=O)N(CCCN(C)C)Cc3ccccn3)cc1C(C)C)OCO2] | 834 [M + H]⁺ |
| 185 | tert-Butyl (5r,7r)-2-(1-(4- ((4-(4- (dimethylamino) butanamido) phenyl)carbamoyl)- 2-isopropylphenyl)-5-(5- methoxybenzo[d][1,3] dioxol-4-yl)-1H-pyrazole-3- carboxamido)adamantane- 2-carboxylate | 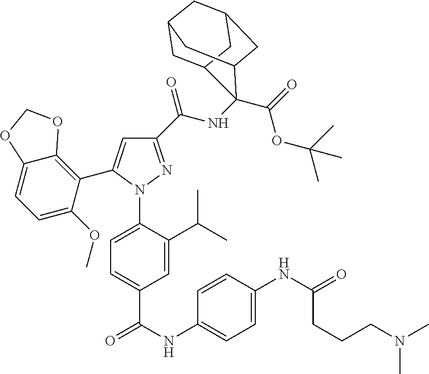 [SMILES: COc1ccc2c(c1-c1cc(C(=O)NC3(C(=O)OC(C)(C)C)C4CC5CC(C4)CC3C5)nn1-c1ccc(C(=O)Nc3ccc(NC(=O)CCCN(C)C)cc3)cc1C(C)C)OCO2] | 861 [M + H]⁺ |
| 186 | tert-Butyl (5r, 7r)-2-(1-(4- ((3-(4- (dimethylamino) butanamido) phenyl)carbamoyl)- 2-isopropylphenyl)-5-(5- methoxybenzo[d][1,3] dioxol-4-yl)-1H-pyrazole-3- carboxamido)adamantane- 2-carboxylate | 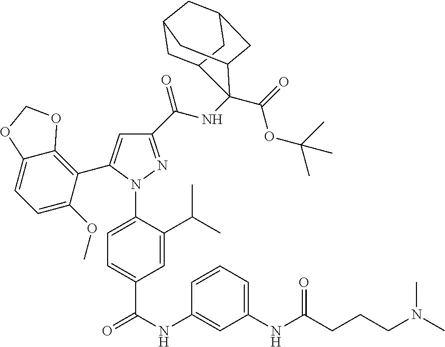 [SMILES: COc1ccc2c(c1-c1cc(C(=O)NC3(C(=O)OC(C)(C)C)C4CC5CC(C4)CC3C5)nn1-c1ccc(C(=O)Nc3cccc(NC(=O)CCCN(C)C)c3)cc1C(C)C)OCO2] | 861 [M + H]⁺ |

### Preparation 187

### tert-Butyl (5r,7r)-2-(1-(6-chloro-4-isopropylpyridin-3-yl)-5-(5-methoxybenzo[d][1,3]dioxol-4-yl)-1H-pyrazole-3-carboxamido)adamantane-2-carboxylate

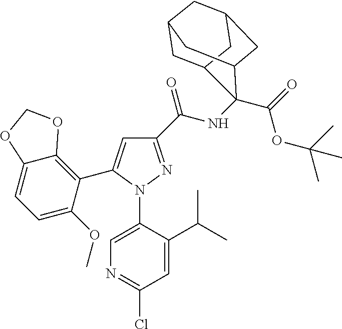 [SMILES: COc1ccc2c(c1-c1cc(C(=O)NC3(C(=O)OC(C)(C)C)C4CC5CC(C4)CC3C5)nn1-c1cnc(Cl)cc1C(C)C)OCO2]

A soln of 1-(6-chloro-4-isopropylpyridin-3-yl)-5-(5-methoxybenzo[d][1,3]dioxol-4-yl)-1H-pyrazole-3-carboxylic acid (170 mg, 0.409 mmol) in DMF (4 mL) was treated with tert-butyl (5r,7r)-2-aminoadamantane-2-carboxylate (154 mg, 0.613 mmol), DIEA (0.356 mL, 2.04 mmol) and HATU (466 mg, 1.23 mmol). After stirring ON, the mixture was treated with water (3 mL), and extracted with iPrOH/CHCl₃ (1:3) (4×5 mL). The combined organic layers were washed with brine and concentrated to afford crude title compound (170 mg, 79.9%) as a brown solid. MS ES+ m/z 649 [M+H]⁺.

### Preparation 32

### tert-Butyl (5r,7r)-2-(1-(4-amino-2-isopropylphenyl)-5-(5-methoxybenzo[d][1,3]dioxol-4-yl)-1H-pyrazole-3-carboxamido)adamantane-2-carboxylate

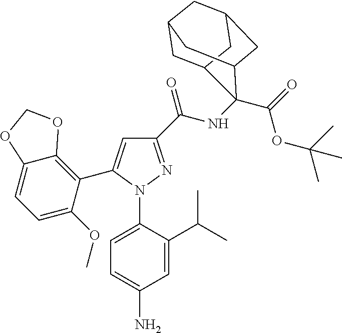 [SMILES: COc1ccc2c(c1-c1cc(C(=O)NC3(C(=O)OC(C)(C)C)C4CC5CC(C4)CC3C5)nn1-c1ccc(N)cc1C(C)C)OCO2]

A soln of tert-butyl (5r,7r)-2-(1-(4-iodo-2-isopropylphenyl)-5-(5-methoxybenzo[d][1,3]dioxol-4-yl)-1H-pyrazole-3-carboxamido)adamantane-2-carboxylate (250 mg, 0.338 mmol) and (2S)-pyrrolidine-2-carboxylic acid (59 mg, 0.507 mmol) in DMSO (3 mL) was treated with NH₃·H₂O (0.1 mL) and CuI (129 mg, 0.676 mmol) under N₂ at RT. After stirring under N₂ at 100° C. for 2 h the mixture was cooled to RT, concentrated, and purified by reversed-phase flash chromatography with the following conditions: column, C18; mobile phase, 90% to 95% ACN in H₂O (0.1% NH₃·H₂O) to afford title compound (170 mg, 79.9%) as a white solid. MS ES+ m/z 629 [M+H]⁺.

### Preparation 33

### tert-Butyl (5r,7r)-2-(1-(4-((5-(dimethylamino) pentyl)amino)-2-isopropylphenyl)-5-(5-methoxybenzo[d][1,3]dioxol-4-yl)-1H-pyrazole-3-carboxamido)adamantane-2-carboxylate

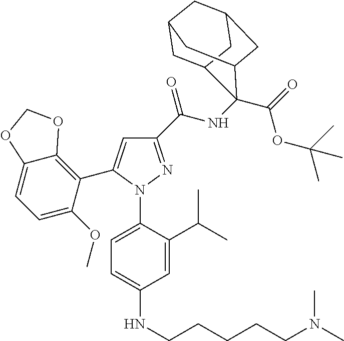 [SMILES: COc1ccc2c(c1-c1cc(C(=O)NC3(C(=O)OC(C)(C)C)C4CC5CC(C4)CC3C5)nn1-c1ccc(NCCCCCN(C)C)cc1C(C)C)OCO2]

A soln of N1,N1-dimethylpentane-1,5-diamine (46 μL, 0.30 mmol), tert-butyl (5r,7r)-2-(1-(4-iodo-2-isopropylphenyl)-5-(5-methoxybenzo[d][1,3]dioxol-4-yl)-1H-pyrazole-3-carboxamido)adamantane-2-carboxylate (30 mg, 41 μmol), Cs₂CO₃ (40 mg, 0.12 mmol), XPhos (1.9 mg, 4.1 μmol), Pd₂(dba)₃ (3.7 mg, 4.1 μmol), in 1,4-Dioxane (1 mL) was degassed with argon for 2 minutes and stirred at 120° C. for 17 h before being concentrated to afford title compound (30 mg, 100%). MS ES+ m/z 743 [M+H]⁺.

The following compounds were prepared in a manner essentially analogous to the preparation method of tert-Butyl (5r,7r)-2-(1-(4-((5-(dimethylamino) pentyl)amino)-2-isopropylphenyl)-5-(5-methoxybenzo[d][1,3]dioxol-4-yl)-1H-pyrazole-3-carboxamido)adamantane-2-carboxylate using the appropriate reagents (the starting aryl halide is preferably bromo or iodo), adjusting temperature, adjusting reaction time to determine completion of the reaction, and adjusting the purification system as appropriate.

TABLE 8

| Prep | | | |
|---|---|---|---|
| # | Chemical Name | Structure | MS ES+ m/z |
| 34 | tert-Butyl (5r,7r)-2-(1- (2-isopropyl-4- (methyl(3-(methyl(3- (methylamino)propyl) amino)propyl)amino) phenyl)-5-(5- methoxybenzo[d][1,3] dioxol-4-yl)-1H- pyrazole-3- carboxamido)adamantane- 2-carboxylate | 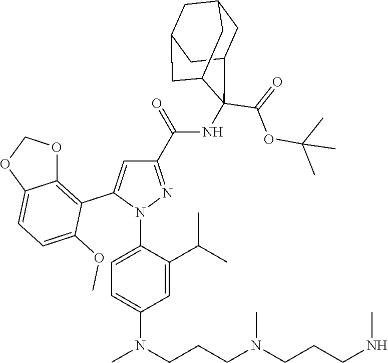 [SMILES: CNCCCN(C)CCCN(C)c1ccc(-n2nc(C(=O)NC3(C(=O)OC(C)(C)C)C4CC5CC(C4)CC3C5)cc2-c2c(OC)ccc3c2OCO3)c(C(C)C)c1] | 786 [M + H]⁺ |
| 35 | tert-Butyl (5r,7r)-2-(1- (4-((3-((3- aminopropyl)(methyl) amino)propyl)amino)-2- isopropylphenyl)-5-(5- methoxybenzo[d][1,3] dioxol-4-yl)-1H- pyrazole-3- carboxamido)adamantane- 2-carboxylate | 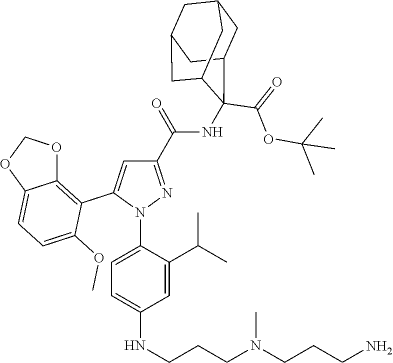 [SMILES: COc1ccc2c(c1-c1cc(C(=O)NC3(C(=O)OC(C)(C)C)C4CC5CC(C4)CC3C5)nn1-c1ccc(NCCCN(C)CCCN)cc1C(C)C)OCO2] | 758 [M + H]⁺ |
| 36 | tert-Butyl (5r,7r)-2-(1- (4-((3- (dimethylamino)propyl) amino)-2- isopropylphenyl)-5-(5- methoxybenzo[d][1,3] dioxol-4-yl)-1H- pyrazole-3- carboxamido)adamantane- 2-carboxylate | 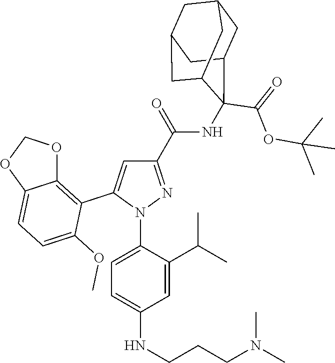 [SMILES: COc1ccc2c(c1-c1cc(C(=O)NC3(C(=O)OC(C)(C)C)C4CC5CC(C4)CC3C5)nn1-c1ccc(NCCCN(C)C)cc1C(C)C)OCO2] | 715 [M + H]⁺ |
| 37 | tert-Butyl (5r,7r)-2-(1- (2-(tert-butyl)-4-((3- (dimethylamino)propyl) amino)phenyl)-5-(5- methoxybenzo[d][1,3] dioxol-4-yl)-1H- pyrazole-3- carboxamido)adamantane- 2-carboxylate | 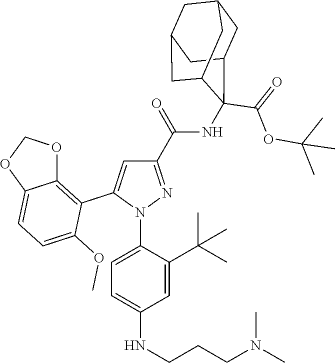 [SMILES: COc1ccc2c(c1-c1cc(C(=O)NC3(C(=O)OC(C)(C)C)C4CC5CC(C4)CC3C5)nn1-c1ccc(NCCCN(C)C)cc1C(C)(C)C)OCO2] | 729 [M + H]⁺ |
| 188 | tert-Butyl 9-((4-(3- (((5r,7r)-2-(tert- butoxycarbonyl) adamantan- 2-yl)carbamoyl)- 5-(5- methoxybenzo[d][1,3] dioxol-4-yl)-1H-pyrazol- 1-yl)-3- isopropylphenyl)amino)- 3- azaspiro[5.5]undecane- 3-carboxylate | 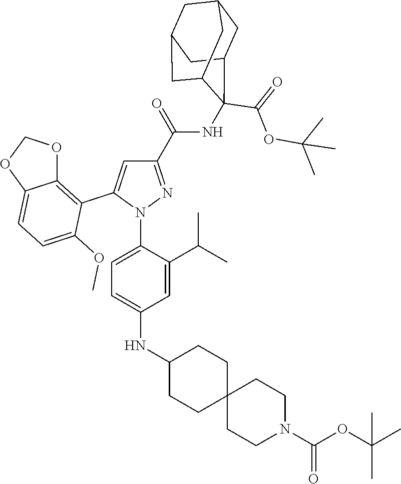 [SMILES: COc1ccc2c(c1-c1cc(C(=O)NC3(C(=O)OC(C)(C)C)C4CC5CC(C4)CC3C5)nn1-c1ccc(NC3CCC4(CC3)CCN(C(=O)OC(C)(C)C)CC4)cc1C(C)C)OCO2] | 881 [M + H]⁺ |
| 189 | tert-Butyl (5r,7r)-2-(1- (2-isopropyl-4-(9- methyl-3,9- diazaspiro[5.5]undecan- 3-yl)phenyl)-5-(5- methoxybenzo[d][1,3] dioxol-4-yl)-1H- pyrazole-3- carboxamido)adamantane- 2-carboxylate | 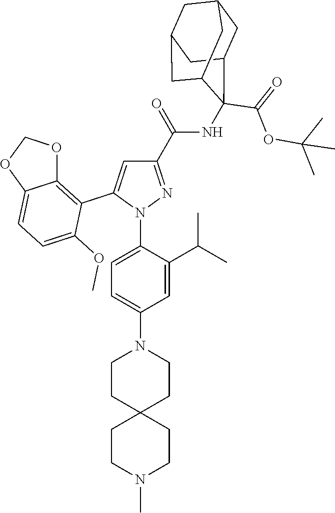 [SMILES: COc1ccc2c(c1-c1cc(C(=O)NC3(C(=O)OC(C)(C)C)C4CC5CC(C4)CC3C5)nn1-c1ccc(N3CCC4(CCN(C)CC4)CC3)cc1C(C)C)OCO2] | 781 [M + H]⁺ |
| 190 | tert-Butyl (5r,7r)-2-(1- (2-isopropyl-4-((3- methyl-3- azaspiro[5.5]undecan- 9-yl)amino)phenyl)-5- (5- methoxybenzo[d][1,3] dioxol-4-yl)-1H- pyrazole-3- carboxamido)adamantane- 2-carboxylate | 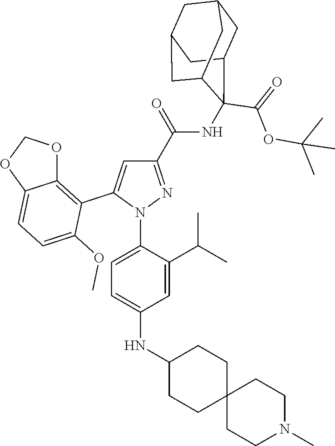 [SMILES: COc1ccc2c(c1-c1cc(C(=O)NC3(C(=O)OC(C)(C)C)C4CC5CC(C4)CC3C5)nn1-c1ccc(NC3CCC4(CC3)CCN(C)CC4)cc1C(C)C)OCO2] | 795 [M + H]⁺ |
| 191 | tert-Butyl (5r,7r)-2-(1- (2-isopropyl-4- (methyl(3- (methylamino)propyl) amino)phenyl)-5-(5- methoxybenzo[d][1,3] dioxol-4-yl)-1H- pyrazole-3- carboxamido)adamantane- 2-carboxylate | 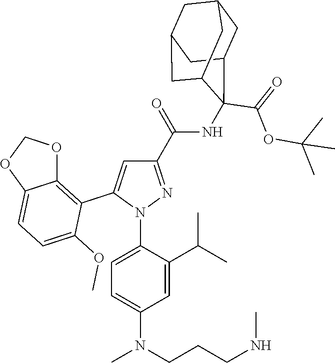 [SMILES: CNCCCN(C)c1ccc(-n2nc(C(=O)NC3(C(=O)OC(C)(C)C)C4CC5CC(C4)CC3C5)cc2-c2c(OC)ccc3c2OCO3)c(C(C)C)c1] | 715 [M + H]⁺ |
| 192 | tert-Butyl (5r,7r)-2-(1- (2-isopropyl-4-(4- methylpiperazin-1- yl)phenyl)-5-(5- methoxybenzo[d][1,3] dioxol-4-yl)-1H- pyrazole-3- carboxamido)adamantane- 2-carboxylate | 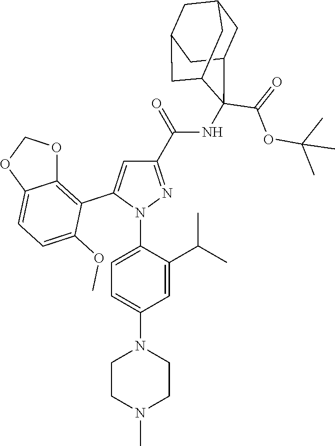 [SMILES: COc1ccc2c(c1-c1cc(C(=O)NC3(C(=O)OC(C)(C)C)C4CC5CC(C4)CC3C5)nn1-c1ccc(N3CCN(C)CC3)cc1C(C)C)OCO2] | 713 [M + H]⁺ |
| 193 | tert-Butyl 4-(4-(3- (((5r,7r)-2-(tert- butoxycarbonyl) adamantan- 2-yl)carbamoyl)- 5-(5- methoxybenzo[d][1,3] dioxol-4-yl)-1H-pyrazol- 1-yl)-3- isopropylphenyl) piperazine- 1-carboxylate | 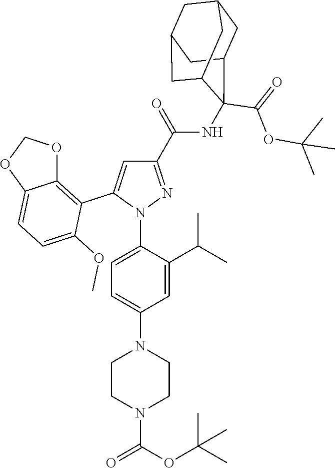 [SMILES: COc1ccc2c(c1-c1cc(C(=O)NC3(C(=O)OC(C)(C)C)C4CC5CC(C4)CC3C5)nn1-c1ccc(N3CCN(C(=O)OC(C)(C)C)CC3)cc1C(C)C)OCO2] | 742 [M − tBu + H]⁺ |
| 194 | tert-Butyl (5r,7r)-2-(1- (4-((4-(4- (dimethylamino) butanamido) phenyl)amino)-2- isopropylphenyl)-5-(5- methoxybenzo[d][1,3] dioxol-4-yl)-1H- pyrazole-3- carboxamido)adamantane- 2-carboxylate | 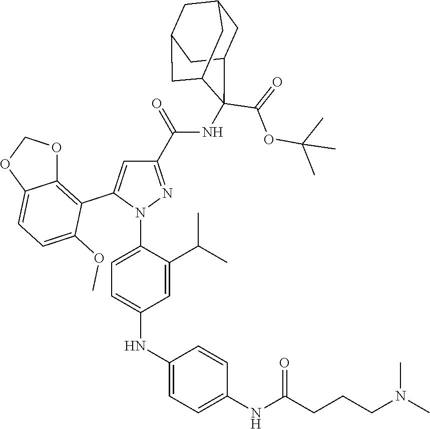 [SMILES: COc1ccc2c(c1-c1cc(C(=O)NC3(C(=O)OC(C)(C)C)C4CC5CC(C4)CC3C5)nn1-c1ccc(Nc3ccc(NC(=O)CCCN(C)C)cc3)cc1C(C)C)OCO2] | 834 [M + H]⁺ |
| 195 | tert-Butyl (5r,7r)-2-(1- (4-((2-cyanoethyl)(3- (dimethylamino)propyl) amino)-2- isopropylphenyl)-5-(5- methoxybenzo[d][1,3] dioxol-4-yl)-1H- pyrazole-3- carboxamido)adamantane- 2-carboxylate | 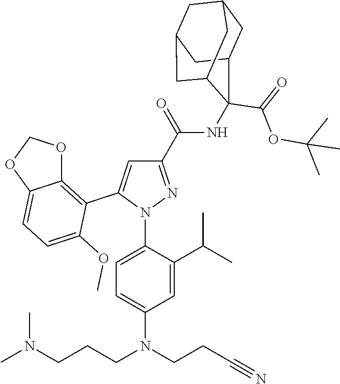 [SMILES: COc1ccc2c(c1-c1cc(C(=O)NC3(C(=O)OC(C)(C)C)C4CC5CC(C4)CC3C5)nn1-c1ccc(N(CCC#N)CCCN(C)C)cc1C(C)C)OCO2] | 768 [M + H]⁺ |
| 196 | tert-Butyl (5r,7r)-2-(1- (2-isopropyl-4- (methyl(3-(methyl(3- (methylamino)propyl) amino)propyl)amino) phenyl)-5-(5- methoxybenzo[d][1,3] dioxol-4-yl)-1H- pyrazole-3- carboxamido)adamantane- 2-carboxylate | 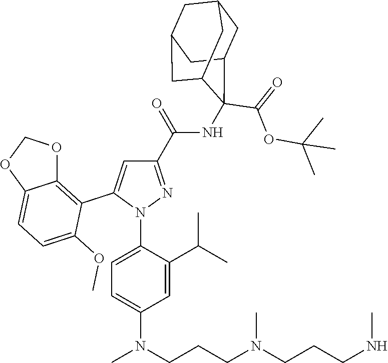 [SMILES: CNCCCN(C)CCCN(C)c1ccc(-n2nc(C(=O)NC3(C(=O)OC(C)(C)C)C4CC5CC(C4)CC3C5)cc2-c2c(OC)ccc3c2OCO3)c(C(C)C)c1] | 786 [M + H]⁺ |
| 312 | tert-Butyl (1R,4R)-5- (4-(3-(((5R,7R)-2-(tert- butoxycarbonyl) adamantan- 2-yl)carbamoyl)- 5-(5- methoxybenzo[d][1,3] dioxol-4-yl)-1H-pyrazol- 1-yl)-3- isopropylphenyl)-2,5- diazabicyclo[2.2.1] heptane-2-carboxylate | 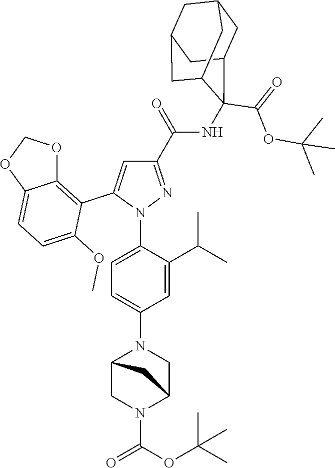 [SMILES: COc1ccc2c(c1-c1cc(C(=O)NC3(C(=O)OC(C)(C)C)C4CC5CC(C4)CC3C5)nn1-c1ccc(N3C[C@H]4C[C@@H]3CN4C(=O)OC(C)(C)C)cc1C(C)C)OCO2] | 810 [M + H]⁺ |
| 313 | tert-Butyl (1S,4S)-5-(4- (3-(((5S,7S)-2-(tert- butoxycarbonyl) adamantan- 2-yl)carbamoyl)- 5-(5- methoxybenzo[d][1,3] dioxol-4-yl)-1H-pyrazol- 1-yl)-3- isopropylphenyl)-2,5- diazabicyclo[2.2.1] heptane-2-carboxylate | 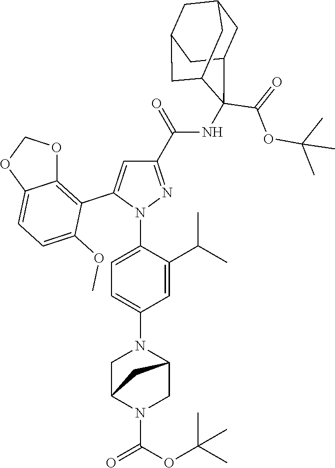 [SMILES: COc1ccc2c(c1-c1cc(C(=O)NC3(C(=O)OC(C)(C)C)C4CC5CC(C4)CC3C5)nn1-c1ccc(N3C[C@@H]4C[C@H]3CN4C(=O)OC(C)(C)C)cc1C(C)C)OCO2] | 811 [M + H]⁺ |
| 314 | tert-Butyl 7-(4-(3- (((5r,7r)-2-(tert- butoxycarbonyl) adamantan- 2-yl)carbamoyl)- 5-(5- methoxybenzo[d][1,3] dioxol-4-yl)-1H-pyrazol- 1-yl)-3- isopropylphenyl)-2,7- diazaspiro[3.5]nonane- 2-carboxylate | 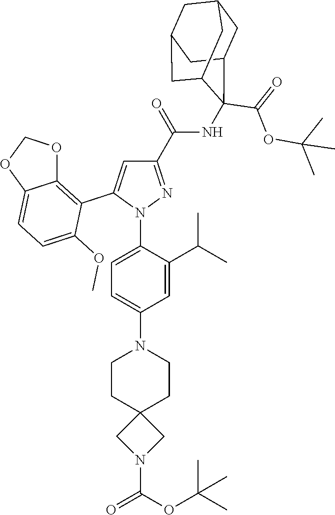 [SMILES: COc1ccc2c(c1-c1cc(C(=O)NC3(C(=O)OC(C)(C)C)C4CC5CC(C4)CC3C5)nn1-c1ccc(N3CCC4(CC3)CN(C(=O)OC(C)(C)C)C4)cc1C(C)C)OCO2] | 839 [M + H]⁺ |
| 315 | tert-Butyl 2-(4-(3- (((5r,7r)-2-(tert- butoxycarbonyl) adamantan- 2-yl)carbamoyl)- 5-(5- methoxybenzo[d][1,3] dioxol-4-yl)-1H-pyrazol- 1-yl)-3- isopropylphenyl)-2,7- diazaspiro[3.5]nonane- 7-carboxylate | 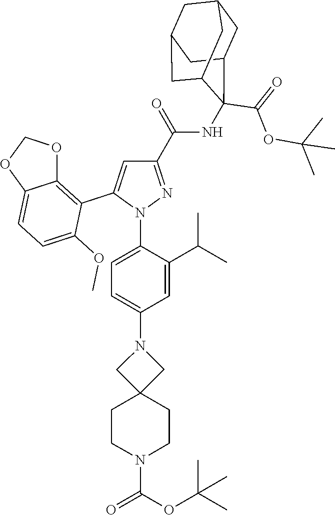 [SMILES: COc1ccc2c(c1-c1cc(C(=O)NC3(C(=O)OC(C)(C)C)C4CC5CC(C4)CC3C5)nn1-c1ccc(N3CC4(CCN(C(=O)OC(C)(C)C)CC4)C3)cc1C(C)C)OCO2] | 839 [M + H]⁺ |
| 316 | tert-utyl (5r,7r)-2-(1-(4- (4-(3-((3- (((benzyloxy)carbonyl) amino)propyl)(methyl) amino)propyl)piperazin- 1-yl)-2- isopropylphenyl)-5-(5- methoxybenzo[d][1,3] dioxol-4-yl)-1H- pyrazole-3- carboxamido)adamantane- 2-carboxylate | 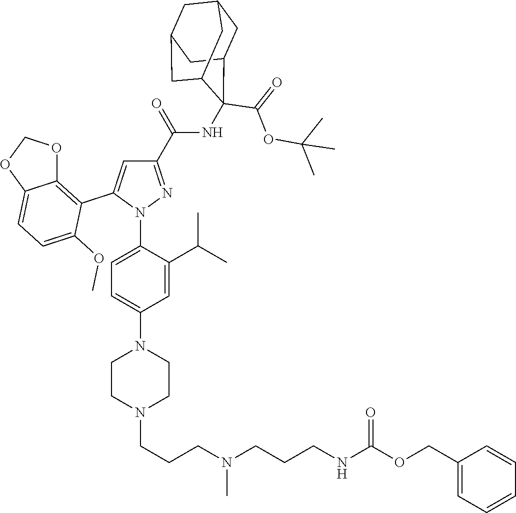 [SMILES: COc1ccc2c(c1-c1cc(C(=O)NC3(C(=O)OC(C)(C)C)C4CC5CC(C4)CC3C5)nn1-c1ccc(N3CCN(CCCN(C)CCCNC(=O)OCc4ccccc4)CC3)cc1C(C)C)OCO2] | 961 [M + H]⁺ |
| 317 | (5r,7r)-2-(1-(2- isopropyl-4-(3,9- diazaspiro[5.5]undecan- 3-yl)phenyl)-5-(5- methoxybenzo[d][1,3] dioxol-4-yl)-1H- pyrazole-3- carboxamido)adamantane- 2-carboxylic acid | 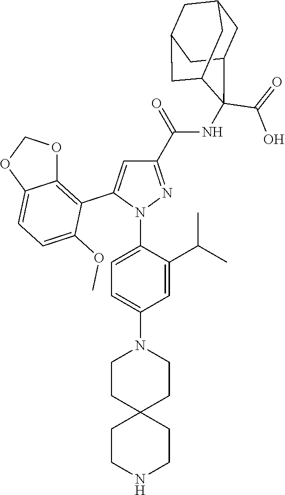 | 767 [M + H]⁺ |
| 318 | tert-Butyl (5r,7r)-2-(1- (4-((3-(4-(3- aminopropyl)piperazin- 1-yl)propyl)amino)-2- isopropylphenyl)-5-(5- methoxybenzo[d][1,3] dioxol-4-yl)-1H- pyrazole-3- carboxamido)adamantane- 2-carboxylate | 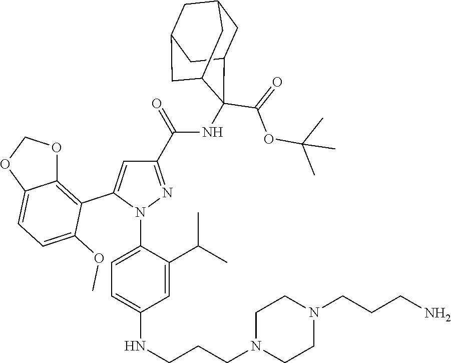 [SMILES: COc1ccc2c(c1-c1cc(C(=O)NC3(C(=O)OC(C)(C)C)C4CC5CC(C4)CC3C5)nn1-c1ccc(NCCCN3CCN(CCCN)CC3)cc1C(C)C)OCO2] | 811 [M + H]⁺ |
| 319 | tert-Butyl (5r,7r)-2-(1- (4-((3-((4-((3- aminopropyl)(methyl) amino)butyl)(methyl) amino)propyl)amino)-2- isopropylphenyl)-5-(5- methoxybenzo[d][1,3] dioxol-4-yl)-1H- pyrazole-3- carboxamido)adamantane- 2-carboxylate | 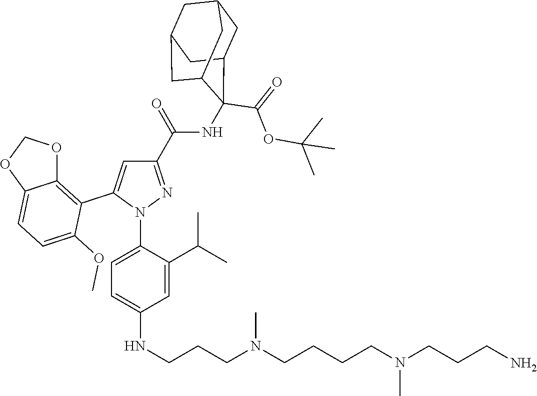 [SMILES: COc1ccc2c(c1-c1cc(C(=O)NC3(C(=O)OC(C)(C)C)C4CC5CC(C4)CC3C5)nn1-c1ccc(NCCCN(C)CCCCN(C)CCCN)cc1C(C)C)OCO2] | 842 [M + H]⁺ |
| 320 | tert-Butyl 9-(4-(3- (((5r,7r)-2-(tert- butoxycarbonyl) adamantan- 2-yl)carbamoyl)- 5-(5- methoxybenzo[d][1,3] dioxol-4-yl)-1H-pyrazol- 1-yl)-3- isopropylphenyl)-8- oxo-3,9- diazaspiro[5.5]undecane- 3-carboxylate | 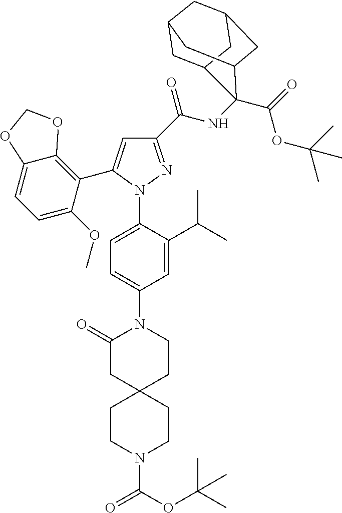 [SMILES: COc1ccc2c(c1-c1cc(C(=O)NC3(C(=O)OC(C)(C)C)C4CC5CC(C4)CC3C5)nn1-c1ccc(N3CCC4(CCN(C(=O)OC(C)(C)C)CC4)CC3=O)cc1C(C)C)OCO2] | 825 [M + H]⁺ |

### Preparation 390

### tert-Butyl (5R,7R)-2-(1-(4-((1R,4R)-2,5-diazabicyclo[2.2.1]heptan-2-yl)-2-isopropylphenyl)-5-(5-methoxybenzo[d][1,3]dioxol-4-yl)-1H-pyrazole-3-carboxamido)adamantane-2-carboxylate

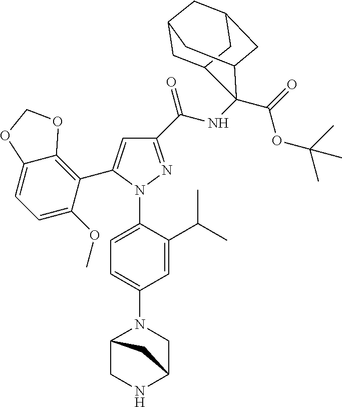 [SMILES: COc1ccc2c(c1-c1cc(C(=O)NC3(C(=O)OC(C)(C)C)C4CC5CC(C4)CC3C5)nn1-c1ccc(N3C[C@H]4C[C@@H]3CN4)cc1C(C)C)OCO2]

To a soln of tert-butyl (1R,4R)-5-(4-((M)-3-(((2r,5S,7S)-2-(tert-butoxycarbonyl)adamantan-2-yl)carbamoyl)-5-(5-methoxybenzo[d][1,3]dioxol-4-yl)-1H-pyrazol-1-yl)-3-isopropylphenyl)-2,5-diazabicyclo[2.2.1]heptane-2-carboxylate (345 mg, 0.43 mmol) in THF (3 mL) and MeOH (0.6 mL) was added TMS (0.28 mL, 2.17 mmol). After stirring for 16 h at RT the mixture was filtered and purified by reversed-phase flash chromatography with the following conditions: column, C18; mobile phase, 10% to 100% ACN in H₂O (0.1% FA) to provide title compound (163 mg, 54%) as a white solid. MS ES+ m/z 711 [M+H]⁺.

The following compounds were prepared in a manner essentially analogous to the preparation method of tert-Butyl (5R,7R)-2-(1-(4-((1R,4R)-2,5-diazabicyclo[2.2.1]heptan-2-yl)-2-isopropylphenyl)-5-(5-methoxybenzo[d][1,3]dioxol-4-yl)-1H-pyrazole-3-carboxamido)adamantane-2-carboxylate using the appropriate reagents (the starting aryl halide is preferably bromo or iodo), adjusting temperature, adjusting reaction time to determine completion of the reaction, and adjusting the purification system as appropriate.

TABLE 51

| Prep | | | MS ES+ |
|---|---|---|---|
| # | Chemical Name | Structure | m/z |
| 321 | tert-Butyl (5S,7S)-2-(1- (4-((1S,4S)-2,5- diazabicyclo[2.2.1]heptan- 2-yl)-2- isopropylphenyl)-5-(5- methoxybenzo[d][1,3] dioxol-4-yl)-1H-pyrazole-3- carboxamido)adamantane- 2-carboxylate | 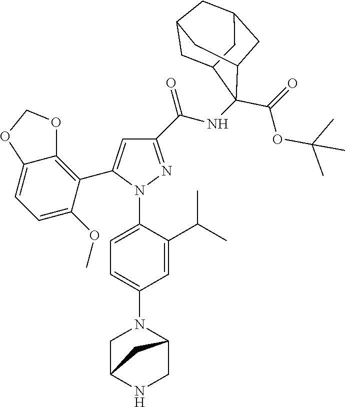 [SMILES: COc1ccc2c(c1-c1cc(C(=O)NC3(C(=O)OC(C)(C)C)C4CC5CC(C4)CC3C5)nn1-c1ccc(N3C[C@@H]4C[C@H]3CN4)cc1C(C)C)OCO2] | 711 [M + H]⁺ |
| 322 | tert-Butyl (5r,7r)-2-(1-(2- isopropyl-4-(2,7- diazaspiro[3.5]nonan-7- yl)phenyl)-5-(5- methoxybenzo[d][1,3] dioxol-4-yl)-1H-pyrazole-3- carboxamido)adamantane- 2-carboxylate | 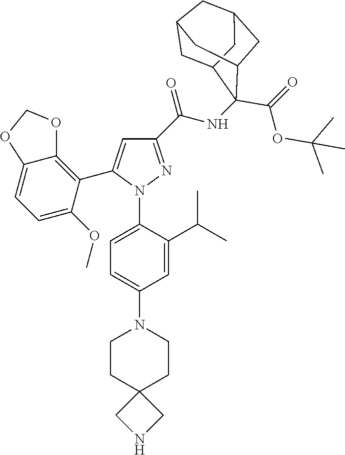 [SMILES: COc1ccc2c(c1-c1cc(C(=O)NC3(C(=O)OC(C)(C)C)C4CC5CC(C4)CC3C5)nn1-c1ccc(N3CCC4(CC3)CNC4)cc1C(C)C)OCO2] | 739 [M + H]⁺ |

### Preparation 391

### tert-Butyl (5r,7r)-2-(1-(2-isopropyl-4-(2,7-diazaspiro[3.5]nonan-2-yl)phenyl)-5-(5-methoxybenzo[d][1,3]dioxol-4-yl)-1H-pyrazole-3-carboxamido)adamantane-2-carboxylate

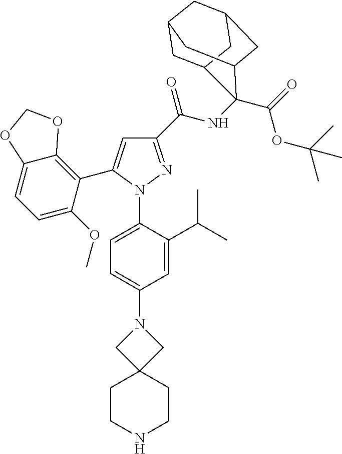 [SMILES: COc1ccc2c(c1-c1cc(C(=O)NC3(C(=O)OC(C)(C)C)C4CC5CC(C4)CC3C5)nn1-c1ccc(N3CC4(CCNCC4)C3)cc1C(C)C)OCO2]

To a soln of tert-butyl 2-(4-((M)-3-(((2r,5S,7S)-2-(tert-butoxycarbonyl)adamantan-2-yl)carbamoyl)-5-(5-methoxybenzo[d][1,3]dioxol-4-yl)-1H-pyrazol-1-yl)-3-isopropylphenyl)-2,7-diazaspiro[3.5]nonane-7-carboxylate (222 mg, 0.265 mmol) in MeCN (3 mL) was added pTsOH. H₂O (101 mg, 0.53 mmol) and stirred at RT for 16 hours. An additional pTsOH. H₂O (101 mg, 0.53 mmol) was added and the soln stirred for 4 hours. The mixture was treated with sat NaHCO₃ and extracted with CHCl₃/IPA (3:1). The aq layer was separated and extracted CHCl₃/IPA (3:1) (×2). The combined organic layers were concentrated under reduced pressure, dried under a stream of N₂, dissolved in DMSO (3 mL), and purified by reversed-phase flash chromatography with the following conditions: column, C18; mobile phase, 10 to 100% ACN in H₂O (0.1% FA) to afford title compound (100 mg, 51%) as an off-white semisolid. MS ES+ m/z 734 [M+H]⁺.

### Preparation 392

### (5r,7r)-2-(1-(2-Isopropyl-4-(2-oxo-3,9-diazaspiro[5.5]undecan-3-yl)phenyl)-5-(5-methoxybenzo[d][1,3]dioxol-4-yl)-1H-pyrazole-3-carboxamido)adamantane-2-carboxylic acid

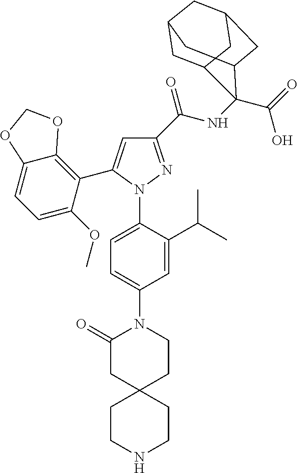 [SMILES: COc1ccc2c(c1-c1cc(C(=O)NC3(C(=O)O)C4CC5CC(C4)CC3C5)nn1-c1ccc(N3CCC4(CCNCC4)CC3=O)cc1C(C)C)OCO2]

A soln of tert-butyl 9-(4-(3-(((1R,3S,5r,7r)-2-(tert-butoxycarbonyl)adamantan-2-yl)carbamoyl)-5-(5-methoxybenzo[d][1,3]dioxol-4-yl)-1H-pyrazol-1-yl)-3-isopropylphenyl)-8-oxo-3,9-diazaspiro[5.5]undecane-3-carboxylate (200 mg, 0.227 mmol) in DCM (1 mL) was treated with TFA (1 mL). The mixture was stirred at RT for 1 h and concentrated to afford title compound (164 mg, 100%). MS ES+ m/z 725 [M+H]⁺.

### Preparation 197

### tert-Butyl (5r,7r)-2-(1-(4-((3-aminopropyl)(3-(dimethylamino)propyl)amino)-2-isopropylphenyl)-5-(5-methoxybenzo[d][1,3]dioxol-4-yl)-1H-pyrazole-3-carboxamido)adamantane-2-carboxylate

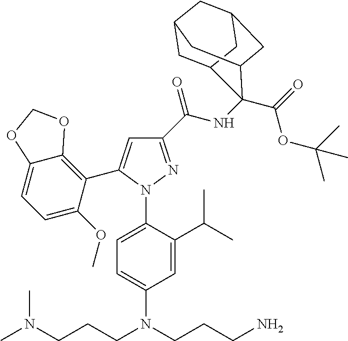 [SMILES: COc1ccc2c(c1-c1cc(C(=O)NC3(C(=O)OC(C)(C)C)C4CC5CC(C4)CC3C5)nn1-c1ccc(N(CCCN)CCCN(C)C)cc1C(C)C)OCO2]

A soln of tert-butyl 2-(1-{4-[(2-cyanoethyl) [3-(dimethylamino)propyl]amino]-2-isopropylphenyl}-5-(5-methoxy-2H-1,3-benzodioxol-4-yl)pyrazole-3-amido)adamantane-2-carboxylate (900 mg, 1.17 mmol) in MeOH (10 mL) was treated with Nickel (100 mg) and NH₄OH (0.5 mL). After stirring at RT ON under H₂, mixture was filtered and the filter cake washed with MeOH (3×5 mL). The filtrate was concentrated and purified by reversed-phase flash chromatography with the following conditions: column, C18; mobile phase, 40 to 60% MeOH in H₂O (0.1% FA) to afford title compound (550 mg, 60.7%) as a yellow solid. MS ES+ m/z 772 [M+H]⁺.

### Preparation 198

### tert-Butyl rac-(5r,7r)-2-(1-(4-((3-(6-((((9H-fluoren-9-yl)methoxy)carbonyl)amino)-2-(4-((4-(4-iodophenyl)butanamido)methyl)benzamido)hexanamido)propyl)(3-(dimethylamino)propyl)amino)-2-isopropylphenyl)-5-(5-methoxybenzo[d][1,3]dioxol-4-yl)-1H-pyrazole-3-carboxamido)adamantane-2-carboxylate

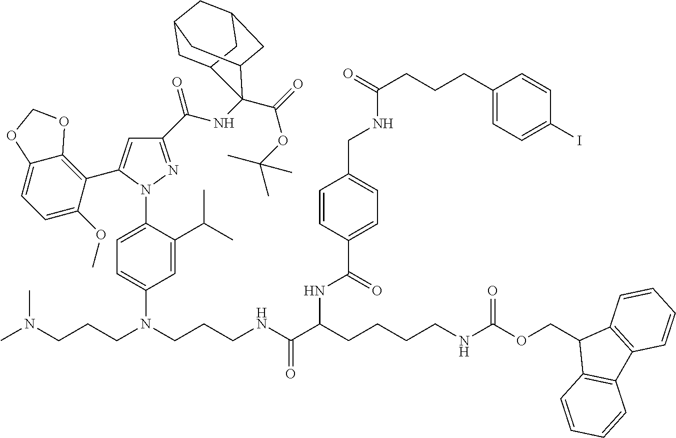 [SMILES: COc1ccc2c(c1-c1cc(C(=O)NC3(C(=O)OC(C)(C)C)C4CC5CC(C4)CC3C5)nn1-c1ccc(N(CCCNC(=O)C(CCCCNC(=O)OCC3c4ccccc4-c4ccccc43)NC(=O)c3ccc(CNC(=O)CCCc4ccc(I)cc4)cc3)CCCN(C)C)cc1C(C)C)OCO2]

A soln of (2S)-6-{[(9H-fluoren-9-ylmethoxy)carbonyl]amino}-2-[(4-{[4-(4-iodophenyl)butanamido]methyl}phenyl)formamido]hexanoic acid (662 mg, 0.85 mmol) in DCM (8 mL) was treated with EDCI (221 mg, 1.42 mmol) and HOBT (289 mg, 2.13 mmol). After stirring at RT for 30 min under N₂, the mixture was treated with tert-butyl 2-(1-{4-[(3-aminopropyl) [3-(dimethylamino)propyl]amino]-2-isopropylphenyl}-5-(5-methoxy-2H-1,3-benzodioxol-4-yl)pyrazole-3-amido)adamantane-2-carboxylate (550 mg, 0.71 mmol). After stirring at RT for 2 h, the mixture was diluted with H₂O (100 mL) and extracted with DCM (3×50 mL). The combined organic layers were washed with brine (1×100 mL), dried over Na₂SO₄, filtered, concentrated, and purified by reversed-phase flash chromatography with the following conditions: column, C18; mobile phase, 40 to 60% ACN in H₂O (0.1% FA) to afford title compound (450 mg, 41.3%) as an off-white solid. MS ES+ m/z 1527/1528 [M+H]⁺.

### Preparation 199

### tert-Butyl (5r,7r)-2-(1-(4-((3-(6-((((9H-fluoren-9-yl)methoxy)carbonyl)amino)-2-(4-((4-(4-iodophenyl)butanamido)methyl)benzamido)hexanamido)propyl)(3-(dimethylamino)propyl)amino)-2-isopropylphenyl)-5-(5-methoxybenzo[d][1,3]dioxol-4-yl)-1H-pyrazole-3-carboxamido)adamantane-2-carboxylate; Isomer 1 & Isomer 2

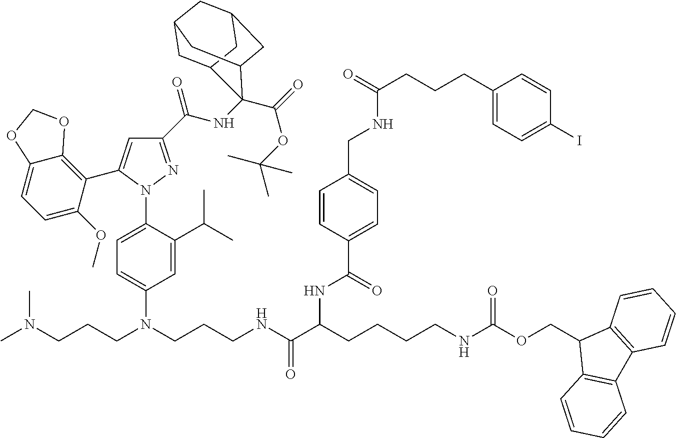 [SMILES: COc1ccc2c(c1-c1cc(C(=O)NC3(C(=O)OC(C)(C)C)C4CC5CC(C4)CC3C5)nn1-c1ccc(N(CCCNC(=O)C(CCCCNC(=O)OCC3c4ccccc4-c4ccccc43)NC(=O)c3ccc(CNC(=O)CCCc4ccc(I)cc4)cc3)CCCN(C)C)cc1C(C)C)OCO2]

Tert-butyl rac-2-[1-(4-{[3-(dimethylamino)propyl]({3-[(2R)-6-{[(9H-fluoren-9-ylmethoxy)carbonyl]amino}-2-[(4-{[4-(4-iodophenyl)butanamido]methyl}phenyl)formamido]hexanamido]propyl}) amino}-2-isopropylphenyl)-5-(5-methoxy-2H-1,3-benzodioxol-4-yl)pyrazole-3-amido]adamantane-2-carboxylate (500 mg) was purified by PREP-CHIRAL-HPLC with the following conditions: Column, CHIRALPAK SZ OBD 3*25 cm, 5 μm; Mobile Phase, 35% Hex (0.1% TFA) in EtOH/DCM (1:1) (0.1% DEA+0.1% TFA). The first eluting peak (Isomer 1) fractions were concentrated to afford title Isomer 1 compound (135 mg, 30%) as an off-white solid MS ES+ m/z 1528 [M+H]⁺. The first eluting peak (Isomer 1) fractions were concentrated to afford title Isomer 1 compound (135 mg, 30%) as an off-white solid MS ES+ m/z 1528 [M+H]⁺. The second eluting peak (Isomer 2) fractions were concentrated to afford title Isomer 2 compound (135 mg, 30%) as an off-white solid MS ES+ m/z 1528 [M+H]⁺. The first eluting peak (Isomer 1) fractions were concentrated to afford title Isomer 1 compound (140 mg, 31.1%) as an off-white solid MS ES+ m/z 1527 [M+H]⁺.

### Preparation 38

### tert-Butyl (5r,7r)-2-(1-(4-hydroxy-2-isopropylphenyl)-5-(5-methoxybenzo[d][1,3]dioxol-4-yl)-1H-pyrazole-3-carboxamido)adamantane-2-carboxylate

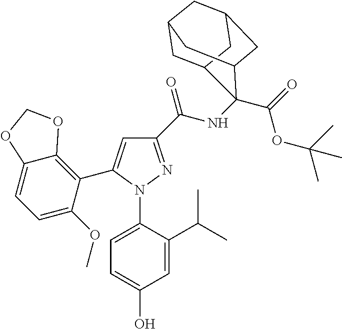 [SMILES: COc1ccc2c(c1-c1cc(C(=O)NC3(C(=O)OC(C)(C)C)C4CC5CC(C4)CC3C5)nn1-c1ccc(O)cc1C(C)C)OCO2]

A reaction vessel was charged with tert-butyl (5r,7r)-2-(1-(4-bromo-2-isopropylphenyl)-5-(5-methoxybenzo[d][1,3]dioxol-4-yl)-1H-pyrazole-3-carboxamido)adamantane-2-carboxylate (500 mg, 722 μmol) and KOH (122 mg, 2.17 mmol). The vessel was degassed under alternating between vacuum and nitrogen (3×). Degassed deionized H₂O (260 mg, 14.4 mmol) and 1,4-Dioxane (6 mL) were added to the vessel. The mixture was charged with t-BuBrettPhos Pd G3 (30.3 mg, 0.036 mmol) and degassed with alternating between vacuum and nitrogen (3×). After stirring at 100° C. for 80 min the mixture was cooled to RT and diluted with sat NH₄Cl aq solution and EA. The layers were separated, and the organic layer was extracted with H₂O (2×). The EA layer was concentrated and purified by silica gel column chromatography EA/Hep (0 to 100%) to afford title compound (250 mg, 55%) as a white solid. MS ES+ m/z 630 [M+H]⁺.

The following compounds were prepared in a manner essentially analogous to the preparation method of tert-butyl (5r,7r)-2-(1-(4-hydroxy-2-isopropylphenyl)-5-(5-methoxybenzo[d][1,3]dioxol-4-yl)-1H-pyrazole-3-carboxamido)adamantane-2-carboxylate using the appropriate reagents (the starting aryl halide is preferably bromo or iodo), adjusting temperature, adjusting reaction time to determine completion of the reaction, and adjusting the purification system as appropriate.

TABLE 37

| Prep | | | MS ES+ |
|---|---|---|---|
| # | Chemical Name | Structure | m/z |
| 200 | tert-Butyl (5r,7r)-2-(1-(4- (2-((2-(1,3- dioxoisoindolin-2- yl)ethyl)(methyl)amino) ethoxy)-2- isopropylphenyl)-5-(5- methoxybenzo[d][1,3] dioxol-4-yl)-1H-pyrazole-3- carboxamido)adamantane- 2-carboxylate | 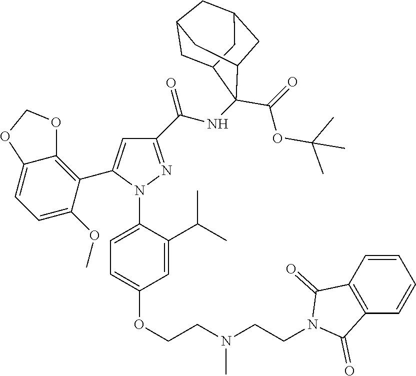 [SMILES: COc1ccc2c(c1-c1cc(C(=O)NC3(C(=O)OC(C)(C)C)C4CC5CC(C4)CC3C5)nn1-c1ccc(OCCN(C)CCN3C(=O)c4ccccc4C3=O)cc1C(C)C)OCO2] | 861 [M + H]⁺ |
| 201 | tert-Butyl (5r,7r)-2-(1-(4- ((3- (dimethylamino)propyl) (methyl)carbamoyl)-2- isopropylphenyl)-5-(5- hydroxybenzo[d][1,3] dioxol-4-yl)-1H-pyrazole-3- carboxamido)adamantane- 2-carboxylate | 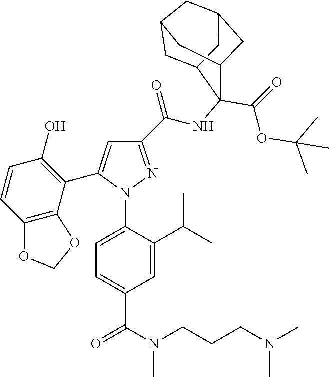 [SMILES: CC(C)c1cc(C(=O)N(C)CCCN(C)C)ccc1-n1nc(C(=O)NC2(C(=O)OC(C)(C)C)C3CC4CC(C3)CC2C4)cc1-c1c(O)ccc2c1OCO2] | 743 [M + H]⁺ |
| 323 | tert-Butyl (5r,7r)-2-(1- (4′-(3-((3-(1,3- dioxoisoindolin-2- yl)propyl)(methyl)amino) propoxy)-3-isopropyl-[ [1,1′-biphenyl]-4-yl)-5- (5- methoxybenzo[d][1,3] dioxol-4-yl)-1H-pyrazole-3- carboxamido)adamantane- 2-carboxylate | 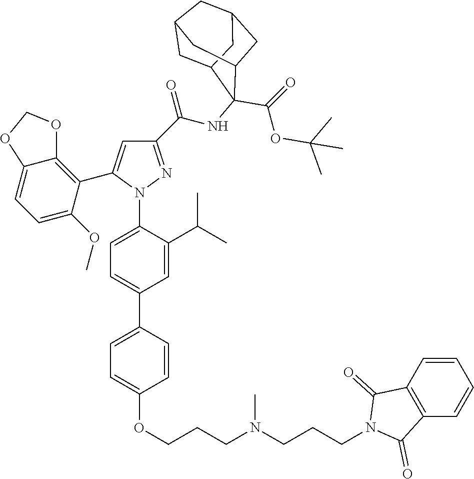 [SMILES: COc1ccc2c(c1-c1cc(C(=O)NC3(C(=O)OC(C)(C)C)C4CC5CC(C4)CC3C5)nn1-c1ccc(-c3ccc(OCCCN(C)CCCN4C(=O)c5ccccc5C4=O)cc3)cc1C(C)C)OCO2] | 965 [M + H]⁺ |
| 324 | Ethyl 5-(5- bromobenzo[d][1,3] dioxol-4-yl)-1-(4-hydroxy-2- isopropylphenyl)-1H- pyrazole-3-carboxylate | 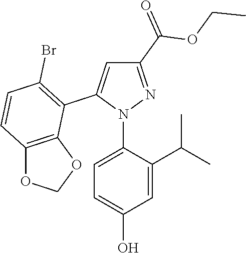 [SMILES: CCOC(=O)c1cc(-c2c(Br)ccc3c2OCO3)n(-c2ccc(O)cc2C(C)C)n1] | 473/475 [M + H]⁺ (⁷⁹Br/⁸¹Br) |

### Preparation 393 (5r,7r)-2-(1-(4-(2-(4-(tert-Butoxycarbonyl)piperazin-1-yl)ethoxy)-2-isopropylphenyl)-5-(5-methoxybenzo[d][1,3]dioxol-4-yl)-1H-pyrazole-3-carboxamido)adamantane-2-carboxylic acid

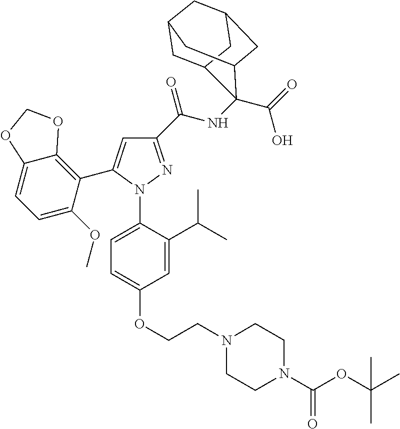 [SMILES: COc1ccc2c(c1-c1cc(C(=O)NC3(C(=O)O)C4CC5CC(C4)CC3C5)nn1-c1ccc(OCCN3CCN(C(=O)OC(C)(C)C)CC3)cc1C(C)C)OCO2]

A soln of 1-Piperazinecarboxylic acid, 4-(2-hydroxyethyl)-, 1,1-dimethylethyl ester (43.9 mg, 0.19 mmol) and tert-butyl(2r,5S,7S)-2-((M)-1-(4-hydroxy-2-isopropylphenyl)-5-(5-methoxybenzo[d][1,3]dioxol-4-yl)-1H-pyrazole-3-carboxamido)adamantane-2-carboxylate (100 mg, 0.16 mmol) in tol (1 mL) was purged with N₂ for 3 min and treated with dropwise addition of (cyanomethylene)tributylphosphorane (0.21 mL, 0.79 mmol). The mixture was stirred for 2 h at 80° C., concentrated under stream of N₂, and purified by reversed-phase flash chromatography with the following conditions: column, C18; mobile phase, 70% to 100% ACN in H₂O to afford title compound (110 mg, 82%) as a white solid. MS ES+ m/z 843 [M+H]⁺.

The following compounds were prepared in a manner essentially analogous to the preparation method of (5r,7r)-2-(1-(4-(2-(4-(tert-Butoxycarbonyl)piperazin-1-yl)ethoxy)-2-isopropylphenyl)-5-(5-methoxybenzo[d][1,3]dioxol-4-yl)-1H-pyrazole-3-carboxamido)adamantane-2-carboxylic acid using the appropriate reagents (the starting aryl halide is preferably bromo or iodo), adjusting temperature, adjusting reaction time to determine completion of the reaction, and adjusting the purification system as appropriate.

TABLE 52

| Prep | | | MS ES+ |
|---|---|---|---|
| # | Chemical Name | Structure | m/z |
| 325 | tert-Butyl (5r,7r)-2-(1-(4- (3-(3-((tert- butoxycarbonyl)amino) propoxy)propoxy)-2- isopropylphenyl)-5-(5- methoxybenzo[d][1,3] dioxol-4-yl)-1H-pyrazole-3- carboxamido)adamantane- 2-carboxylate | 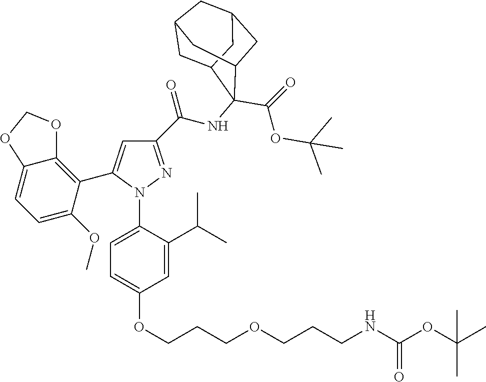 [SMILES: COc1ccc2c(c1-c1cc(C(=O)NC3(C(=O)OC(C)(C)C)C4CC5CC(C4)CC3C5)nn1-c1ccc(OCCCOCCCNC(=O)OC(C)(C)C)cc1C(C)C)OCO2] | 745 [M − Boc + H]⁺ |
| 326 | tert-Butyl 4-(2-(4-(3- (((5r,7r)-2-(tert- butoxycarbonyl)adamantan- 2-yl)carbamoyl)-5-(5- methoxybenzo[d][1,3] dioxol-4-yl)-1H-pyrazol-1- yl)-3- isopropylphenoxy)ethyl) piperidine-1-carboxylate | 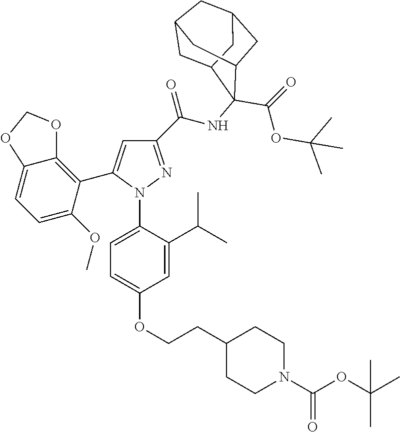 [SMILES: COc1ccc2c(c1-c1cc(C(=O)NC3(C(=O)OC(C)(C)C)C4CC5CC(C4)CC3C5)nn1-c1ccc(OCCC3CCN(C(=O)OC(C)(C)C)CC3)cc1C(C)C)OCO2] | 741 [M − Boc + H]⁺ |
| 327 | tert-Butyl 4-(3-(4-(3- (((5r,7r)-2-(tert- butoxycarbonyl)adamantan- 2-yl)carbamoyl)-5-(5- methoxybenzo[d][1,3] dioxol-4-yl)-1H-pyrazol-1- yl)-3- isopropylphenoxy)propyl) piperazine-1-carboxylate | 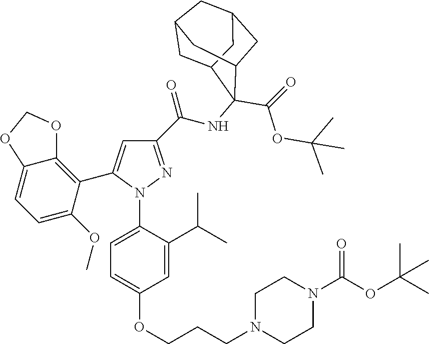 [SMILES: COc1ccc2c(c1-c1cc(C(=O)NC3(C(=O)OC(C)(C)C)C4CC5CC(C4)CC3C5)nn1-c1ccc(OCCCN3CCN(C(=O)OC(C)(C)C)CC3)cc1C(C)C)OCO2] | 857 [M + H]⁺ |

### Preparation 394

### tert-Butyl (5r,7r)-2-(1-(2-isopropyl-4-(2-(piperidin-4-yl)ethoxy)phenyl)-5-(5-methoxybenzo[d][1,3]dioxol-4-yl)-1H-pyrazole-3-carboxamido)adamantane-2-carboxylate

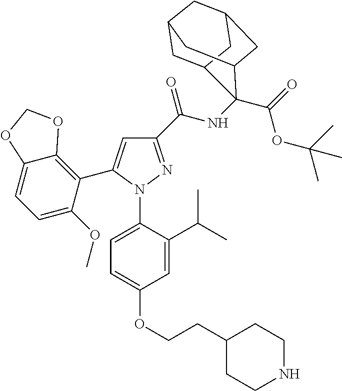 [SMILES: COc1ccc2c(c1-c1cc(C(=O)NC3(C(=O)OC(C)(C)C)C4CC5CC(C4)CC3C5)nn1-c1ccc(OCCC3CCNCC3)cc1C(C)C)OCO2]

A soln of tert-butyl 4-(2-(4-(3-(((1R,3S,5r,7r)-2-(tert-butoxycarbonyl)adamantan-2-yl)carbamoyl)-5-(5-methoxybenzo[d][1,3]dioxol-4-yl)-1H-pyrazol-1-yl)-3-isopropylphenoxy) ethyl)piperidine-1-carboxylate (378.4 mg, 0.45 mmol) in THF (4 mL) and MeOH (0.8 mL) was treated with TMSCl (0.29 mL, 2.25 mmol) and stirred for 18 hours. The mixture was concentrated under a stream of N₂ and purified by reversed-phase flash chromatography with the following conditions: column, C18; mobile phase, 30% to 100% ACN in H₂O to afford title compound (259 mg, 78%) as a brown solid. MS ES+ m/z 742 [M+H]⁺.

### Preparation 202

### tert-Butyl (5r,7r)-2-(1-(4-((3-(dimethylamino)propyl)(methyl)carbamoyl)-2-isopropylphenyl)-5-(5-(2-methoxyethoxy)benzo[d][1,3]dioxol-4-yl)-1H-pyrazole-3-carboxamido)adamantane-2-carboxylate

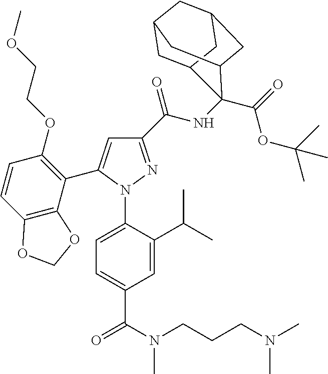 [SMILES: COCCOc1ccc2c(c1-c1cc(C(=O)NC3(C(=O)OC(C)(C)C)C4CC5CC(C4)CC3C5)nn1-c1ccc(C(=O)N(C)CCCN(C)C)cc1C(C)C)OCO2]

A soln of tert-butyl 2-[1-(4-{[3-(dimethylamino)propyl](methyl)carbamoyl}-2-isopropylphenyl)-5-(5-hydroxy-2H-1,3-benzodioxol-4-yl)pyrazole-3-amido]adamantane-2-carboxylate (55 mg, 0.074 mmol) and 2-methoxyethanol (11 mg, 0.148 mmol) in toluene (1 mL) was treated with 2-(tributyl-1\hat{ }[5]-phosphanylidene) acetonitrile (358 mg, 1.480 mmol) at RT under N₂. After stirring at 80° C. ON under N₂, the mixture was concentrated and purified by silica gel column chromatography MeOH/DCM (1:4) to afford title compound (30 mg, 50.59%) as a light yellow solid. MS ES+ m/z 800 [M+H]⁺.

### Preparation 39

### tert-Butyl (5r,7r)-2-(1-(4-(3-(dimethylamino)propoxy)-2-isopropylphenyl)-5-(5-methoxybenzo[d][1,3]dioxol-4-yl)-1H-pyrazole-3-carboxamido)adamantane-2-carboxylate

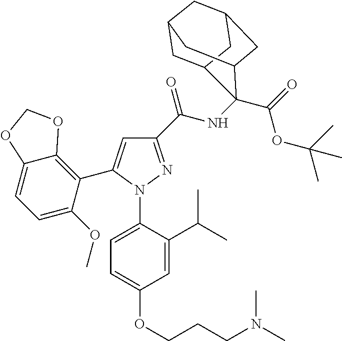 [SMILES: COc1ccc2c(c1-c1cc(C(=O)NC3(C(=O)OC(C)(C)C)C4CC5CC(C4)CC3C5)nn1-c1ccc(OCCCN(C)C)cc1C(C)C)OCO2]

A soln of tert-butyl (5r,7r)-2-(1-(4-bromo-2-isopropylphenyl)-5-(5-methoxybenzo[d][1,3]dioxol-4-yl)-1H-pyrazole-3-carboxamido)adamantane-2-carboxylate (120 mg, 173 μmol), Cs₂CO₃ (169 mg, 520 μmol), CuI (66.0 mg, 346 μmol) in 3-(dimethylamino)propan-1-ol (1.79 g, 17.3 mmol) was flushed with N₂ and heated in a microwave at 130° C. for 36 h. The mixture was concentrated and purified by silica gel column chromatography MeOH/DCM (0 to 50%) to afford title compound (55 mg, 44%) as a green solid. MS ES+ m/z 716 [M+H]⁺.

The following compounds were prepared in a manner essentially analogous to the preparation method of tert-Butyl (5r,7r)-2-(1-(4-(3-(dimethylamino)propoxy)-2-isopropylphenyl)-5-(5-methoxybenzo[d][1,3]dioxol-4-yl)-1H-pyrazole-3-carboxamido)adamantane-2-carboxylate using the appropriate reagents (the starting aryl halide is preferably bromo or iodo), adjusting temperature, adjusting reaction time to determine completion of the reaction, and adjusting the purification system as appropriate.

TABLE 38

| Prep | | | MS ES+ |
|---|---|---|---|
| # | Chemical Name | Structure | m/z |
| 203 | tert-Butyl (5r,7r)-2-(1-(4- (4-(4- (dimethylamino)butanamido) phenoxy)-2- isopropylphenyl)-5-(5- methoxybenzo[d][1,3] dioxol-4-yl)-1H-pyrazole-3- carboxamido)adamantane- 2-carboxylate | 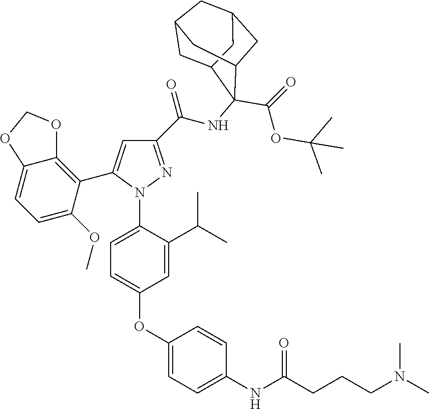 [SMILES: COc1ccc2c(c1-c1cc(C(=O)NC3(C(=O)OC(C)(C)C)C4CC5CC(C4)CC3C5)nn1-c1ccc(Oc3ccc(NC(=O)CCCN(C)C)cc3)cc1C(C)C)OCO2] | 834 [M + H]⁺ |

### Preparation 204

### tert-Butyl (5r,7r)-2-(1-(6-(3-(dimethylamino)propoxy)-4-isopropylpyridin-3-yl)-5-(5-methoxybenzo[d][1,3]dioxol-4-yl)-1H-pyrazole-3-carboxamido)adamantane-2-carboxylate

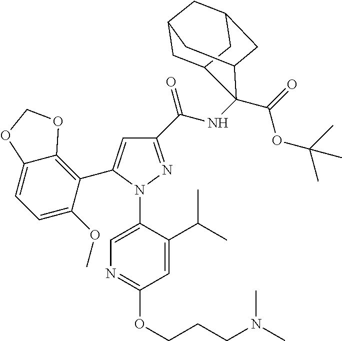 [SMILES: COc1ccc2c(c1-c1cc(C(=O)NC3(C(=O)OC(C)(C)C)C4CC5CC(C4)CC3C5)nn1-c1cnc(OCCCN(C)C)cc1C(C)C)OCO2]

A soln of NaH (22.7 mg, 60% Wt, 0.567 mmol) was in DMA (0.5 mL) at 21° C. under N₂, was treated with 3-(dimethylamino)propan-1-ol (54.8 mg, 0.531 mmol). After stirring for 1 h, the mixture was treated with tert-butyl (5r,7r)-2-(1-(6-chloro-4-isopropylpyridin-3-yl)-5-(5-methoxybenzo[d][1,3]dioxol-4-yl)-1H-pyrazole-3-carboxamido)adamantane-2-carboxylate (230 mg, 0.354 mmol) in DMA (0.5 mL). After stirring at 80° C. for 1 h, the mixture was diluted with H₂O, extracted with DCM, washed with brine and dried over MgSO₄ and concentrated to afford crude title compound (200 mg, 78.9%) as a brown oil. MS ES+ m/z 716 [M+H]⁺.

### Preparation 40

### 4′-(3-(((5r,7r)-2-(tert-Butoxycarbonyl)adamantan-2-yl)carbamoyl)-5-(5-methoxybenzo[d][1,3]dioxol-4-yl)-1H-pyrazol-1-yl)-3′-isopropyl-[1,1′-biphenyl]-4-carboxylic acid

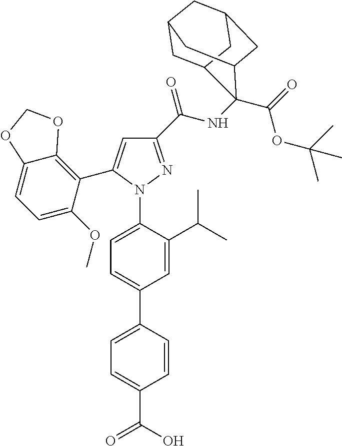 [SMILES: COc1ccc2c(c1-c1cc(C(=O)NC3(C(=O)OC(C)(C)C)C4CC5CC(C4)CC3C5)nn1-c1ccc(-c3ccc(C(=O)O)cc3)cc1C(C)C)OCO2]

A soln of tert-butyl (5r,7r)-2-(1-(4-bromo-2-isopropylphenyl)-5-(5-methoxybenzo[d][1,3]dioxol-4-yl)-1H-pyrazole-3-carboxamido)adamantane-2-carboxylate (150 mg, 0.217 mmol) and 4-carboxyphenylboronic acid, pinacol ester (59.1 mg, 0.238 mmol) in 1,4-Dioxane (3 mL) and H₂O (0.5 mL) under N₂ at RT was treated with K₂CO₃ (89.8 mg, 0.650 mmol) and PdCl₂(PPh₃)₂ (22.8 mg, 0.0325 mmol). The mixture degassed with N₂ for 5 min and then stirred at 100° C. for 1 h before being cooled, diluted with H₂O (10 mL), and extracted with EA (3×15 ml). The combined organic layer was washed with H₂O (10 mL), brine (10 mL), dried over Na₂SO₄, concentrated to dryness, and purified by silica gel column chromatography MeOH/DCM (0 to 50%) to afford title compound (170 mg, 91%) as a brown solid. MS ES+ m/z 678 [M+H]⁺.

The following compounds were prepared in a manner essentially analogous to the preparation method of 4′-(3-(((5r,7r)-2-(tert-Butoxycarbonyl)adamantan-2-yl)carbamoyl)-5-(5-methoxybenzo[d][1,3]dioxol-4-yl)-1H-pyrazol-1-yl)-3′-isopropyl-[1,1′-biphenyl]-4-carboxylic acid using the appropriate reagents (the starting aryl halide is preferably bromo or iodo), adjusting temperature, adjusting reaction time to determine completion of the reaction, and adjusting the purification system as appropriate.

TABLE 9

| Prep | | | MS ES+ |
|---|---|---|---|
| # | Chemical Name | Structure | m/z |
| 41 | 4′-(3-(((5r,7r)-2-(tert- Butoxycarbonyl)adamantan- 2-yl)carbamoyl)-5-(5- methoxybenzo[d][1,3] dioxol-4-yl)-1H-pyrazol-1- yl)-3′-isopropyl-[1,1′- biphenyl]-3-carboxylic acid | 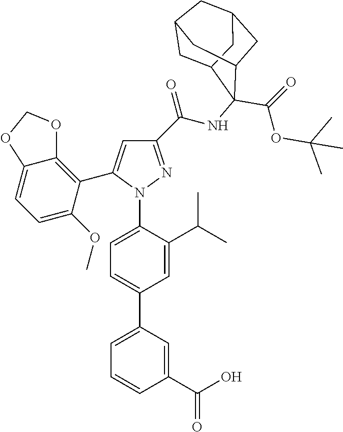 [SMILES: COc1ccc2c(c1-c1cc(C(=O)NC3(C(=O)OC(C)(C)C)C4CC5CC(C4)CC3C5)nn1-c1ccc(-c3cccc(C(=O)O)c3)cc1C(C)C)OCO2] | 678 [M + H]⁺ |
| 42 | tert-Butyl (5r,7r)-2-(1- (4′-(3- (dimethylamino)propoxy)- 3-isopropyl-[1,1′- biphenyl]-4-yl)-5-(5- methoxybenzo[d][1,3] dioxol-4-yl)-1H-pyrazole-3- carboxamido)adamantane- 2-carboxylate | 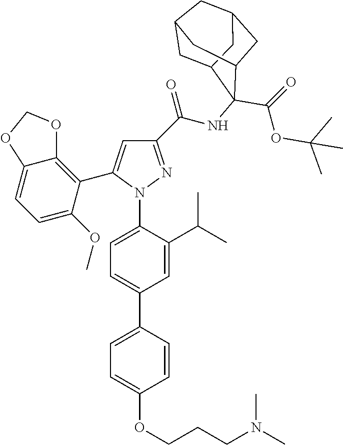 [SMILES: COc1ccc2c(c1-c1cc(C(=O)NC3(C(=O)OC(C)(C)C)C4CC5CC(C4)CC3C5)nn1-c1ccc(-c3ccc(OCCCN(C)C)cc3)cc1C(C)C)OCO2] | 792 [M + H]⁺ |
| 43 | tert-Butyl (5r,7r)-2-(1- (4′-amino-3-isopropyl- [1,1′-biphenyl]-4-yl)-5- (5- methoxybenzo[d][1,3] dioxol-4-yl)-1H-pyrazole-3- carboxamido)adamantane- 2-carboxylate | 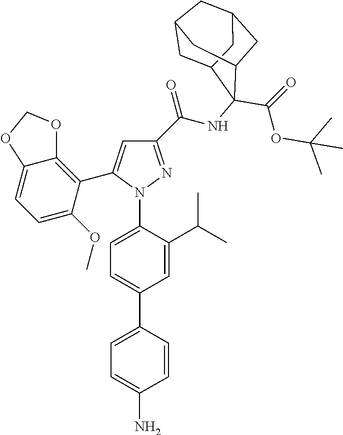 [SMILES: COc1ccc2c(c1-c1cc(C(=O)NC3(C(=O)OC(C)(C)C)C4CC5CC(C4)CC3C5)nn1-c1ccc(-c3ccc(N)cc3)cc1C(C)C)OCO2] | 649 [M − tBuH]⁺ |
| 44 | tert-Butyl (5r,7r)-2-(1- (3′-amino-3-isopropyl- [1,1′-biphenyl]-4-yl)-5- (5- methoxybenzo[d][1,3] dioxol-4-yl)-1H-pyrazole-3- carboxamido)adamantane- 2-carboxylate | 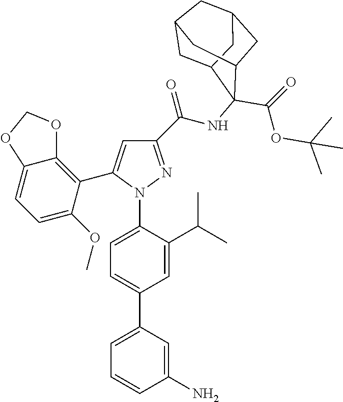 [SMILES: COc1ccc2c(c1-c1cc(C(=O)NC3(C(=O)OC(C)(C)C)C4CC5CC(C4)CC3C5)nn1-c1ccc(-c3cccc(N)c3)cc1C(C)C)OCO2] | 649 [M − tBuH]⁺ |
| 45 | tert-Butyl (5r,7r)-2-(1-(3- isopropyl-4′- (methylamino)-[1,1′- biphenyl]-4-yl)-5-(5- methoxybenzo[d][1,3] dioxol-4-yl)-1H-pyrazole-3- carboxamido)adamantane- 2-carboxylate | 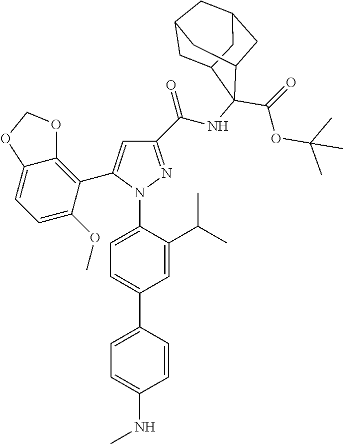 [SMILES: CNc1ccc(-c2ccc(-n3nc(C(=O)NC4(C(=O)OC(C)(C)C)C5CC6CC(C5)CC4C6)cc3-c3c(OC)ccc4c3OCO4)c(C(C)C)c2)cc1] | 664 [M − tBuH]⁺ |
| 205 | tert-Butyl (5r,7r)-2-(1-(2- isopropyl-4-(2-methyl-1- oxo-1,2,3,4- tetrahydroisoquinolin-6- yl)phenyl)-5-(5- methoxybenzo[d][1,3] dioxol-4-yl)-1H-pyrazole-3- carboxamido)adamantane- 2-carboxylate | 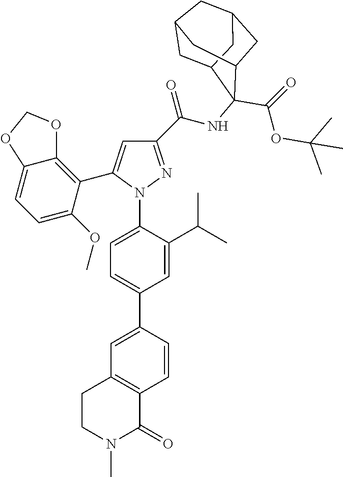 [SMILES: COc1ccc2c(c1-c1cc(C(=O)NC3(C(=O)OC(C)(C)C)C4CC5CC(C4)CC3C5)nn1-c1ccc(-c3ccc4c(c3)CCN(C)C4=O)cc1C(C)C)OCO2] | 774 [M + H]⁺ |
| 206 | tert-Butyl (5r,7r)-2-(1-(2- isopropyl-4-(2-methyl-1- oxoisoindolin-5- yl)phenyl)-5-(5- methoxybenzo[d][1,3] dioxol-4-yl)-1H-pyrazole- 3-carboxamido)adamantane- 2-carboxylate | 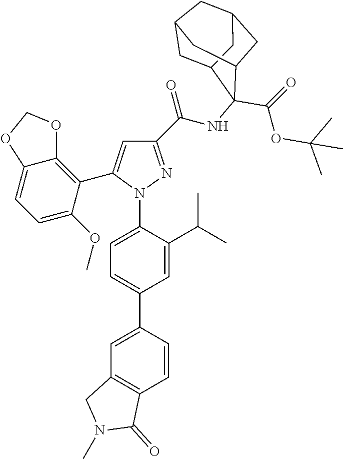 [SMILES: COc1ccc2c(c1-c1cc(C(=O)NC3(C(=O)OC(C)(C)C)C4CC5CC(C4)CC3C5)nn1-c1ccc(-c3ccc4c(c3)CN(C)C4=O)cc1C(C)C)OCO2] | 759 [M + H]⁺ |
| 207 | 5-(4-(3-(((5r,7r)-2-(tert- Butoxycarbonyl)adamantan- 2-yl)carbamoyl)-5-(5- methoxybenzo[d][1,3] dioxol-4-yl)-1H-pyrazol-1- yl)-3- isopropylphenyl)picolinic acid | 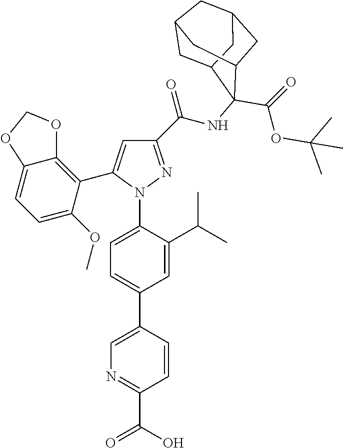 [SMILES: COc1ccc2c(c1-c1cc(C(=O)NC3(C(=O)OC(C)(C)C)C4CC5CC(C4)CC3C5)nn1-c1ccc(-c3ccc(C(=O)O)nc3)cc1C(C)C)OCO2] | 735 [M + H]⁺ |
| 208 | tert-Butyl (5r,7r)-2-(1-(2- isopropyl-4-(2-methyl- 2H-indazol-5-yl)phenyl)- 5-(5- methoxybenzo[d][1,3] dioxol-4-yl)-1H-pyrazole-3- carboxamido)adamantane- 2-carboxylate | 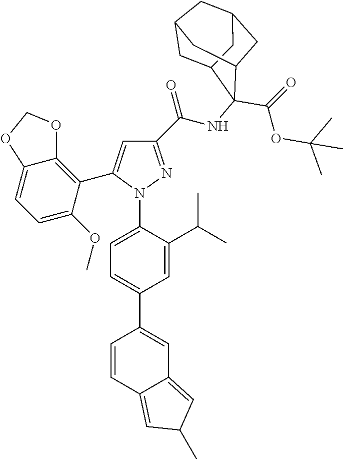 [SMILES: COc1ccc2c(c1-c1cc(C(=O)NC3(C(=O)OC(C)(C)C)C4CC5CC(C4)CC3C5)nn1-c1ccc(-c3ccc4c(c3)=CC(C)C=4)cc1C(C)C)OCO2] | 744 [M + H]⁺ |
| 209 | tert-Butyl 6-(4-(3- (((5r,7r)-2-(tert- butoxycarbonyl)adamantan- 2-yl)carbamoyl)-5-(5- methoxybenzo[d][1,3] dioxol-4-yl)-1H-pyrazol-1- yl)-3-isopropylphenyl)- 3,4-dihydroisoquinoline- 2(1H)-carboxylate | 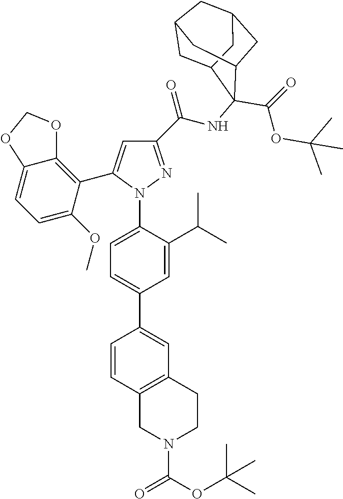 [SMILES: COc1ccc2c(c1-c1cc(C(=O)NC3(C(=O)OC(C)(C)C)C4CC5CC(C4)CC3C5)nn1-c1ccc(-c3ccc4c(c3)CCN(C(=O)OC(C)(C)C)C4)cc1C(C)C)OCO2] | 789 [M − tBu + H]⁺ |
| 210 | tert-Butyl (5r,7r)-2-(1-(2- isopropyl-4-(2-methyl- 1,2,3,4- tetrahydroisoquinolin-6- yl)phenyl)-5-(5- methoxybenzo[d][1,3] dioxol-4-yl)-1H-pyrazole-3- carboxamido)adamantane- 2-carboxylate | 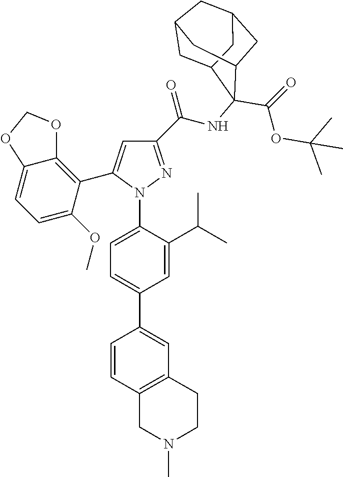 | 780 [M + H]⁺ |
| 211 | tert-Butyl 4-(4-(3- (((5r,7r)-2-(tert- butoxycarbonyl)adamantan- 2-yl)carbamoyl)-5-(5- methoxybenzo[d][1,3] dioxol-4-yl)-1H-pyrazol-1- yl)-3- isopropylphenyl)piperidine- 1-carboxylate | 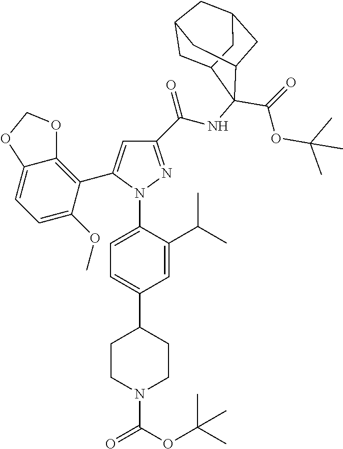 [SMILES: COc1ccc2c(c1-c1cc(C(=O)NC3(C(=O)OC(C)(C)C)C4CC5CC(C4)CC3C5)nn1-c1ccc(C3CCN(C(=O)OC(C)(C)C)CC3)cc1C(C)C)OCO2] | 741 [M − tBu + H]⁺ |
| 212 | tert-Butyl (5r,7r)-2-(1-(2- isopropyl-4-(1-methyl- 1,2,3,6- tetrahydropyridin-4- yl)phenyl)-5-(5- methoxybenzo[d][1,3] dioxol-4-yl)-1H-pyrazole-3- carboxamido)adamantane- 2-carboxylate | 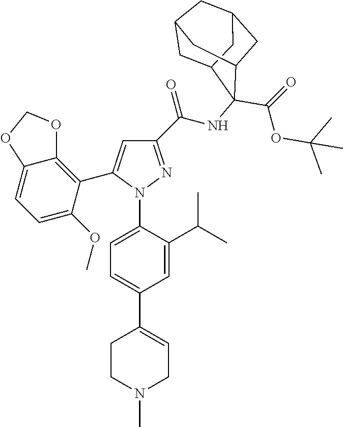 [SMILES: COc1ccc2c(c1-c1cc(C(=O)NC3(C(=O)OC(C)(C)C)C4CC5CC(C4)CC3C5)nn1-c1ccc(C3=CCN(C)CC3)cc1C(C)C)OCO2] | 710 [M + H]⁺ |
| 328 | 5-(4-(3-(((5r,7r)-2-(tert- Butoxycarbonyl)adamantan- 2-yl)carbamoyl)-5-(5- methoxybenzo[d][1,3] dioxol-4-yl)-1H-pyrazol-1- yl)-3-isopropylphenyl)-3- methylpicolinic acid | 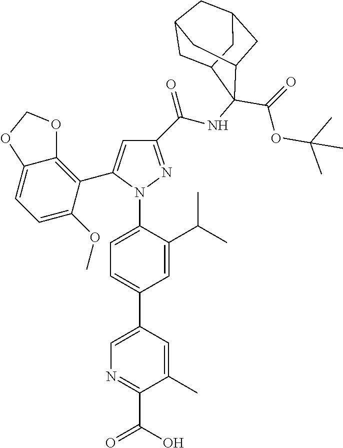 | 697 [M + H]⁺ |
| 329 | tert-Butyl (5r,7r)-2-(1- (4′-bromo-3-isopropyl- [1,1′-biphenyl]-4-yl)-5- (5- methoxybenzo[d][1,3] dioxol-4-yl)-1H-pyrazole-3- carboxamido)adamantane- 2-carboxylate | 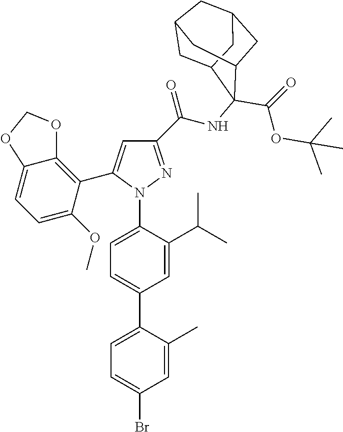 | 714 [M + H]⁺ |

### Preparation 213

### tert-Butyl (5r,7r)-2-(1-(2-isopropyl-4-(1-methylpiperidin-4-yl)phenyl)-5-(5-methoxybenzo[d][1,3]dioxol-4-yl)-1H-pyrazole-3-carboxamido)adamantane-2-carboxylate

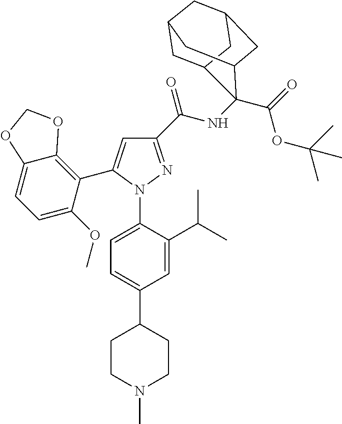 [SMILES: COc1ccc2c(c1-c1cc(C(=O)NC3(C(=O)OC(C)(C)C)C4CC5CC(C4)CC3C5)nn1-c1ccc(C3CCN(C)CC3)cc1C(C)C)OCO2]

A soln tert-butyl 2-{1-[2-isopropyl-4-(1-methyl-3,6-dihydro-2H-pyridin-4-yl)phenyl]-5-(5-methoxy-2H-1,3-benzodioxol-4-yl)pyrazole-3-amido}adamantane-2-carboxylate (80 mg, 0.11 mmol) in MeOH (4 mL) was treated with Pd/C (20 mg, 10%). After stirring at RT ON under H₂, the mixture was filtered and the filter cake washed with methanol (3×4 mL). The filtrate was concentrated and purified by reversed-phase flash chromatography with the following conditions: column, C18; mobile phase, 40 to 60% ACN in H₂O (0.1% FA) to afford title compound (20 mg, 24.94%) as a yellow solid. MS ES+ m/z 712 [M+H]⁺.

### Preparation 214

### tert-Butyl (5r,7r)-2-(1-(2-isopropyl-4-(4,4,5,5-tetramethyl-1,3,2-dioxaborolan-2-yl)phenyl)-5-(5-methoxybenzo[d][1,3]dioxol-4-yl)-1H-pyrazole-3-carboxamido)adamantane-2-carboxylate

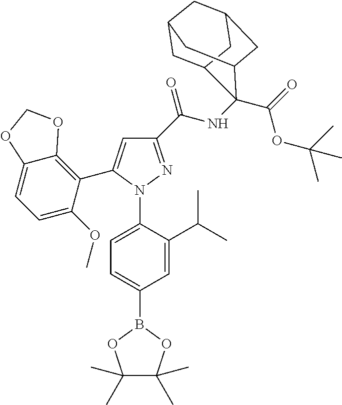 [SMILES: COc1ccc2c(c1-c1cc(C(=O)NC3(C(=O)OC(C)(C)C)C4CC5CC(C4)CC3C5)nn1-c1ccc(B3OC(C)(C)C(C)(C)O3)cc1C(C)C)OCO2]

A soln of tert-butyl 2-[1-(4-bromo-2-isopropylphenyl)-5-(5-methoxy-2H-1,3-benzodioxol-4-yl)pyrazole-3-amido]adamantane-2-carboxylate (7.5 g, 10.828 mmol) and B2pin2 (5.50 g, 21.656 mmol) in Dioxane (100 mL) was treated with Pd(dppf)Cl₂ (792.29 mg, 1.083 mmol) and AcOK (2.66 g, 27.070 mmol) at RT under N₂. After stirring at 80° C. for 6 h, the mixture was allowed to cool to RT, filtered, and the filter cake washed with EtOAc (3×100 mL). The filtrate was concentrated and purified by reversed-phase flash chromatography with the following conditions: column, C18; mobile phase, 95 to 100% ACN in H₂O (0.1% FA) to afford title compound (5 g, 62.4%) as an off-white solid. MS ES+ m/z 741 [M+H]⁺.

### Preparation 215

### tert-Butyl (5r,7r)-2-(1-(4-(5-aminopyridin-2-yl)-2-isopropylphenyl)-5-(5-methoxybenzo[d][1,3]dioxol-4-yl)-1H-pyrazole-3-carboxamido)adamantane-2-carboxylate

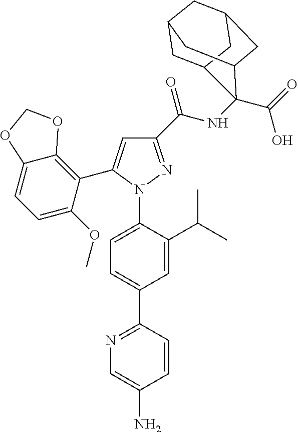 [SMILES: COc1ccc2c(c1-c1cc(C(=O)NC3(C(=O)O)C4CC5CC(C4)CC3C5)nn1-c1ccc(-c3ccc(N)cn3)cc1C(C)C)OCO2]

A soln of tert-butyl (5r,7r)-2-(1-(2-isopropyl-4-(4,4,5,5-tetramethyl-1,3,2-dioxaborolan-2-yl)phenyl)-5-(5-methoxybenzo[d][1,3]dioxol-4-yl)-1H-pyrazole-3-carboxamido)adamantane-2-carboxylate (102 mg, 0.138 mmol) in 1,4-Dioxane (2 mL) was treated with 6-bromopyridin-3-amine (28.6 mg, 0.165 mmol), Na₂CO₃ (29.2 mg, 0.276 mmol) and Pd(dppf)Cl₂·DCM (11.3 mg, 0.0138 mmol). After degassing, the mixture was heated to 95° C. for 18 h. The mixture was cooled to RT, filtered through diatomaceous earth, washed with EtOAc and concentrated to afford crude title compound (90 mg, 92%). MS ES+ m/z 706 [M+H]⁺.

The following compounds were prepared in a manner essentially analogous to the preparation method of tert-Butyl (5r,7r)-2-(1-(4-(5-aminopyridin-2-yl)-2-isopropylphenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 39

| Prep | | | MS ES+ |
|---|---|---|---|
| # | Chemical Name | Structure | m/z |
| 216 | tert-Butyl (5r,7r)-2-(1-(4- (6-aminopyridazin-3-yl)- 2-isopropylphenyl)-5-(5- methoxybenzo[d][1,3] dioxol-4-yl)-1H-pyrazole-3- carboxamido)adamantane- 2-carboxylate | 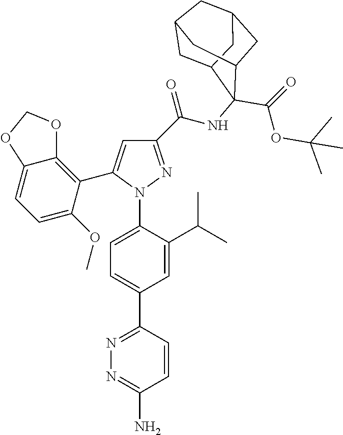 [SMILES: COc1ccc2c(c1-c1cc(C(=O)NC3(C(=O)OC(C)(C)C)C4CC5CC(C4)CC3C5)nn1-c1ccc(-c3ccc(N)nn3)cc1C(C)C)OCO2] | 707 [M + H]⁺ |
| 217 | tert-Butyl (5r,7r)-2-(1-(4- (7-((3- (dimethylamino)propyl) (methyl)carbamoyl)quinolin- 3-yl)-2- isopropylphenyl)-5-(5- methoxybenzo[d][1,3] dioxol-4-yl)-1H-pyrazole-3- carboxamido)adamantane- 2-carboxylate | 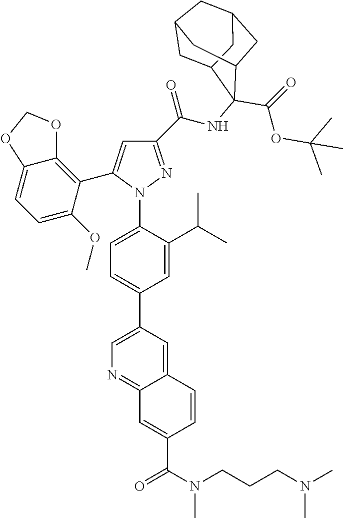 [SMILES: COc1ccc2c(c1-c1cc(C(=O)NC3(C(=O)OC(C)(C)C)C4CC5CC(C4)CC3C5)nn1-c1ccc(-c3cnc4cc(C(=O)N(C)CCCN(C)C)ccc4c3)cc1C(C)C)OCO2] | 884 [M + H]⁺ |
| 218 | tert-Butyl (5r,7r)-2-(1- (3′-cyano-4′-((3- (dimethylamino)propyl) (methyl)carbamoyl)-3- isopropyl-[1,1′- biphenyl]-4-yl)-5-(5- methoxybenzo[d][1,3] dioxol-4-yl)-1H-pyrazole-3- carboxamido)adamantane- 2-carboxylate | 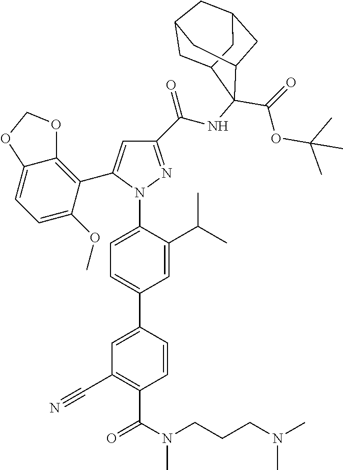 [SMILES: COc1ccc2c(c1-c1cc(C(=O)NC3(C(=O)OC(C)(C)C)C4CC5CC(C4)CC3C5)nn1-c1ccc(-c3ccc(C(=O)N(C)CCCN(C)C)c(C#N)c3)cc1C(C)C)OCO2] | 858 [M + H]⁺ |
| 219 | tert-Butyl (5r,7r)-2-(1- (4′-((3- (dimethylamino)propyl) (methyl)carbamoyl)-3- isopropyl-3′-nitro-[1,1′- biphenyl]-4-yl)-5-(5- methoxybenzo[d][1,3] dioxol-4-yl)-1H-pyrazole-3- carboxamido)adamantane- 2-carboxylate | 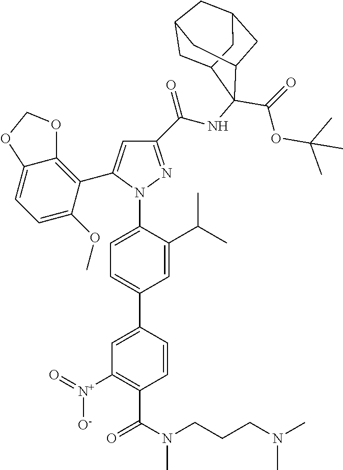 [SMILES: COc1ccc2c(c1-c1cc(C(=O)NC3(C(=O)OC(C)(C)C)C4CC5CC(C4)CC3C5)nn1-c1ccc(-c3ccc(C(=O)N(C)CCCN(C)C)c([N+](=O)[O-])c3)cc1C(C)C)OCO2] | 878 [M + H]⁺ |
| 220 | tert-Butyl (5r,7r)-2-(1-(4- (6-((3- (dimethylamino)propyl) (methyl)carbamoyl) naphthalen-2-yl)-2- isopropylphenyl)-5-(5- methoxybenzo[d][1,3] dioxol-4-yl)-1H-pyrazole-3- carboxamido)adamantane- 2-carboxylate | 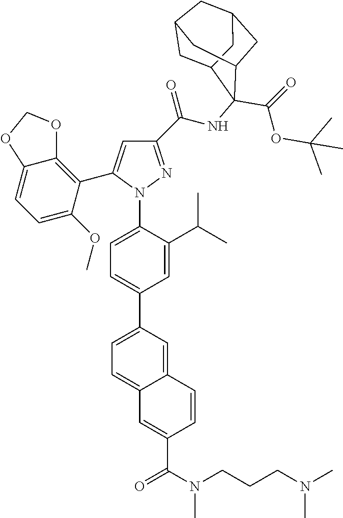 [SMILES: COc1ccc2c(c1-c1cc(C(=O)NC3(C(=O)OC(C)(C)C)C4CC5CC(C4)CC3C5)nn1-c1ccc(-c3ccc4cc(C(=O)N(C)CCCN(C)C)ccc4c3)cc1C(C)C)OCO2] | 883 [M + H]⁺ |
| 221 | tert-Butyl (5r,7r)-2-(1-(2- isopropyl-4-(isoquinolin- 3-yl)phenyl)-5-(5- methoxybenzo[d][1,3] dioxol-4-yl)-1H-pyrazole-3- carboxamido)adamantane- 2-carboxylate | 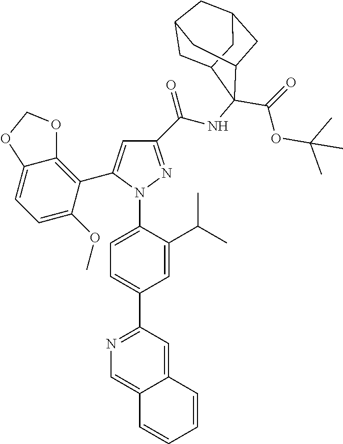 [SMILES: COc1ccc2c(c1-c1cc(C(=O)NC3(C(=O)OC(C)(C)C)C4CC5CC(C4)CC3C5)nn1-c1ccc(-c3cc4ccccc4cn3)cc1C(C)C)OCO2] | 685 [M− tBu + H]⁺ |
| 222 | tert-Butyl (5r,7r)-2-(1-(2- isopropyl-4-(quinolin-3- yl)phenyl)-5-(5- methoxybenzo[d][1,3] dioxol-4-yl)-1H-pyrazole-3- carboxamido)adamantane- 2-carboxylate | 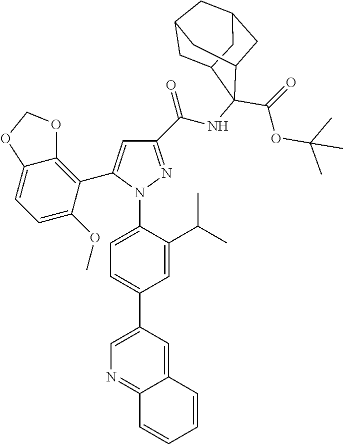 [SMILES: COc1ccc2c(c1-c1cc(C(=O)NC3(C(=O)OC(C)(C)C)C4CC5CC(C4)CC3C5)nn1-c1ccc(-c3cnc4ccccc4c3)cc1C(C)C)OCO2] | 742 [M + H]⁺ |
| 223 | tert-Butyl (5r,7r)-2-(1-(4- (6-((3- (dimethylamino)propyl) (methyl)carbamoyl)-5- methoxynaphthalen-2- yl)-2-isopropylphenyl)-5- (5- methoxybenzo[d][1,3] dioxol-4-yl)-1H-pyrazole-3- carboxamido)adamantane- 2-carboxylate | 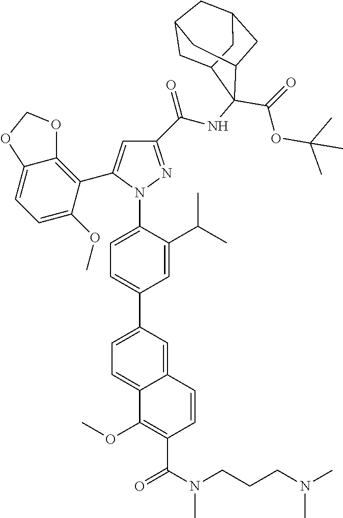 | 863 [M + H]⁺ |
| 224 | tert-Butyl (5r,7r)-2-(1-(4- (1-(3- (dimethylamino)propyl)- 1H-indazol-3-yl)-2- isopropylphenyl)-5-(5- methoxybenzo[d][1,3] dioxol-4-yl)-1H-pyrazole-3- carboxamido)adamantane- 2-carboxylate | 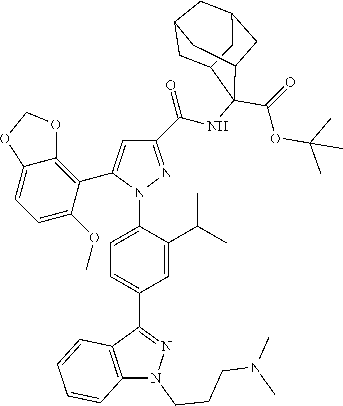 [SMILES: COc1ccc2c(c1-c1cc(C(=O)NC3(C(=O)OC(C)(C)C)C4CC5CC(C4)CC3C5)nn1-c1ccc(-c3nn(CCCN(C)C)c4ccccc34)cc1C(C)C)OCO2] | 816 [M + H]⁺ |
| 225 | 3-(4-(3-(((5r,7r)-2-(tert- Butoxycarbonyl)adamantan- 2-yl)carbamoyl)-5-(5- methoxybenzo[d][1,3] dioxol-4-yl)-1H-pyrazol-1- yl)-3- isopropylphenyl)quinoline- 7-carboxylic acid | 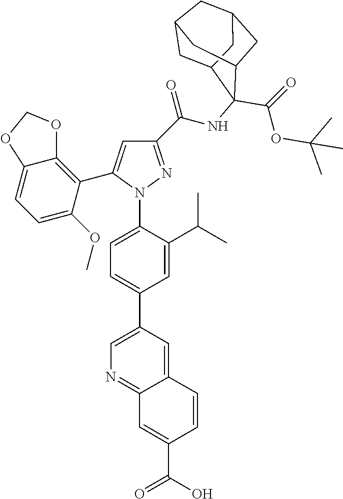 [SMILES: COc1ccc2c(c1-c1cc(C(=O)NC3(C(=O)OC(C)(C)C)C4CC5CC(C4)CC3C5)nn1-c1ccc(-c3cnc4cc(C(=O)O)ccc4c3)cc1C(C)C)OCO2] | 783 [M − H]⁻ |
| 226 | tert-Butyl (5r,7r)-2-(1-(4- (3-((3- (dimethylamino)propyl) (methyl)carbamoyl)quinolin- 7-yl)-2- isopropylphenyl)-5-(5- methoxybenzo[d][1,3] dioxol-4-yl)-1H-pyrazole-3- carboxamido)adamantane- 2-carboxylate | 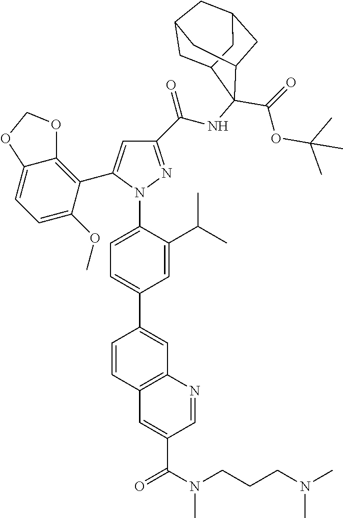 [SMILES: COc1ccc2c(c1-c1cc(C(=O)NC3(C(=O)OC(C)(C)C)C4CC5CC(C4)CC3C5)nn1-c1ccc(-c3ccc4cc(C(=O)N(C)CCCN(C)C)cnc4c3)cc1C(C)C)OCO2] | 884 [M + H]⁺ |
| 330 | 6-(4-(3-(((5r,7r)-2-(tert- Butoxycarbonyl)adamantan- 2-yl)carbamoyl)-5-(5- methoxybenzo[d][1,3] dioxol-4-yl)-1H-pyrazol-1- yl)-3- isopropylphenyl)quinoxaline- 2-carboxylic acid | 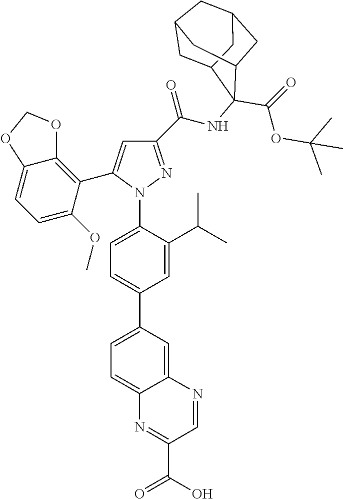 [SMILES: COc1ccc2c(c1-c1cc(C(=O)NC3(C(=O)OC(C)(C)C)C4CC5CC(C4)CC3C5)nn1-c1ccc(-c3ccc4nc(C(=O)O)cnc4c3)cc1C(C)C)OCO2] | 730 [M − tBu + H]⁺ |
| 331 | 7-(4-(3-(((5r,7r)-2-(tert- Butoxycarbonyl)adamantan- 2-yl)carbamoyl)-5-(5- methoxybenzo[d][1,3] dioxol-4-yl)-1H-pyrazol-1- yl)-3- isopropylphenyl)quinoline- 3-carboxylic acid | 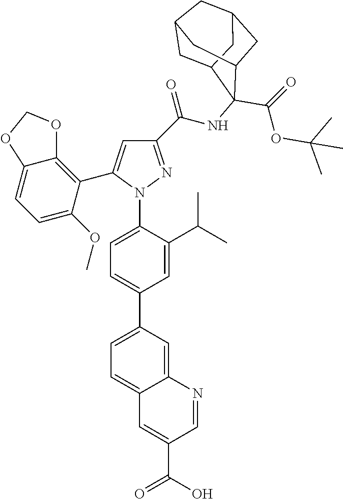 [SMILES: COc1ccc2c(c1-c1cc(C(=O)NC3(C(=O)OC(C)(C)C)C4CC5CC(C4)CC3C5)nn1-c1ccc(-c3ccc4cc(C(=O)O)cnc4c3)cc1C(C)C)OCO2] | 783 [M − H]⁻ |
| 332 | tert-butyl (5r,7r)-2-(1-(4- (7-(3-((3-((tert- butoxycarbonyl)amino) propyl)(methyl)amino) propoxy)quinolin-3-yl)-2- isopropylphenyl)-5-(5- methoxybenzo[d][1,3] dioxol-4-yl)-1H-pyrazole-3- carboxamido)adamantane- 2-carboxylate | 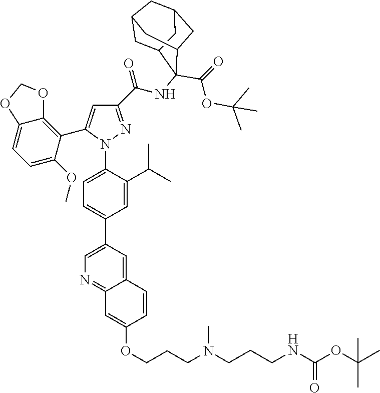 [SMILES: COc1ccc2c(c1-c1cc(C(=O)NC3(C(=O)OC(C)(C)C)C4CC5CC(C4)CC3C5)nn1-c1ccc(-c3cnc4cc(OCCCN(C)CCCNC(=O)OC(C)(C)C)ccc4c3)cc1C(C)C)OCO2] | 986 [M + H]⁺ |
| 333 | 7-(4-(3-(((5r,7r)-2-(tert- Butoxycarbonyl)adamantan- 2-yl)carbamoyl)-5-(5- methoxybenzo[d][1,3] dioxol-4-yl)-1H-pyrazol-1- yl)-3-isopropylphenyl)- 1,5-naphthyridine-3- carboxylic acid | 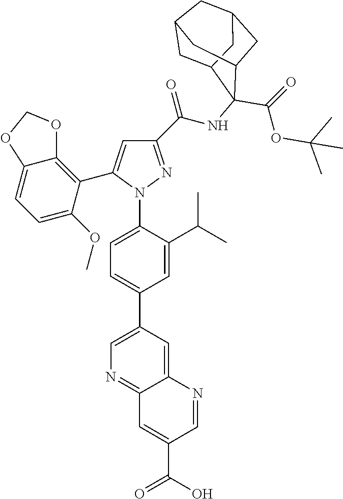 [SMILES: COc1ccc2c(c1-c1cc(C(=O)NC3(C(=O)OC(C)(C)C)C4CC5CC(C4)CC3C5)nn1-c1ccc(-c3cnc4cc(C(=O)O)cnc4c3)cc1C(C)C)OCO2] | 730 [M − ^{t}Bu + H]⁺ |
| 334 | 4′-(3-(((5r,7r)-2-(tert- Butoxycarbonyl)adamantan- 2-yl)carbamoyl)-5-(5- methoxybenzo[d][1,3] dioxol-4-yl)-1H-pyrazol-1- yl)-2,6-difluoro-3′- isopropyl-[1,1′- biphenyl]-4-carboxylic acid | 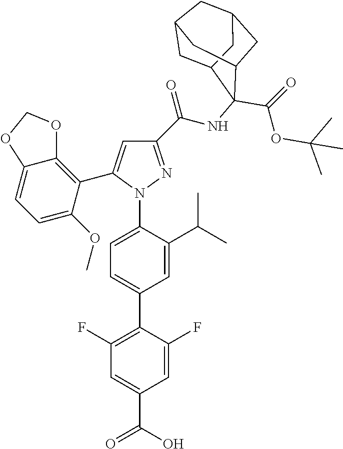 [SMILES: COc1ccc2c(c1-c1cc(C(=O)NC3(C(=O)OC(C)(C)C)C4CC5CC(C4)CC3C5)nn1-c1ccc(-c3c(F)cc(C(=O)O)cc3F)cc1C(C)C)OCO2] | 714 [M − ^{t}Bu + H]⁺ |
| 335 | 4′-(3-(((5r,7r)-2-(tert- Butoxycarbonyl)adamantan- 2-yl)carbamoyl)-5-(5- methoxybenzo[d][1,3] dioxol-4-yl)-1H-pyrazol-1- yl)-3′-isopropyl-3- methoxy-[1,1′-biphenyl]- 4-carboxylic acid | 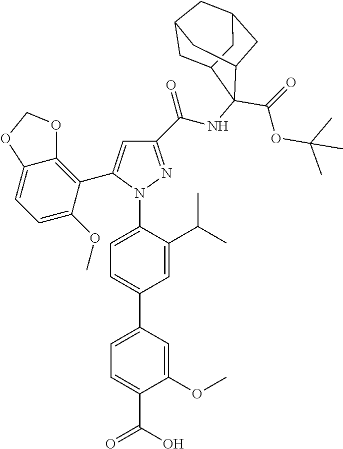 [SMILES: COc1cc(-c2ccc(-n3nc(C(=O)NC4(C(=O)OC(C)(C)C)C5CC6CC(C5)CC4C6)cc3-c3c(OC)ccc4c3OCO4)c(C(C)C)c2)ccc1C(=O)O] | 708 [M − ^{t}Bu + H]⁺ |

### Preparation 227

### 4,4,5,5-Tetramethyl-2-(5-(trifluoromethoxy)benzo[d][1,3]dioxol-4-yl)-1,3,2-dioxaborolane

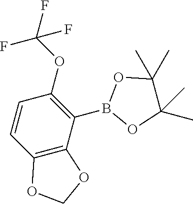 [SMILES: CC1(C)OB(c2c(OC(F)(F)F)ccc3c2OCO3)OC1(C)C]

A soln of 4-bromo-5-(trifluoromethoxy)benzo[d][1,3]dioxole (150 mg, 0.526 mmol), Bis(pinacolato) diborane (200 mg, 0.789 mmol), KOAc (155 mg, 1.58 mmol), [1,1′-Pd(dppf)Cl₂·DCM (21.5 mg, 0.05 Eq, 0.0263 mmol) in 1,4-Dioxane (4 mL). The mixture was purged with N₂ for 5 min. After stirring at 100° C. for 12 h, the reaction was cooled to RT to afford crude title compound (175 mg, 100%). MS ES+ m/z 331 [M+H]⁺.

The following compounds were prepared in a manner essentially analogous to the preparation method of 4,4,5,5-Tetramethyl-2-(5-(trifluoromethoxy)benzo[d][1,3]dioxol-4-yl)-1,3,2-dioxaborolane using the appropriate reagents, adjusting temperature, adjusting reaction time to determine completion of the reaction, and adjusting the purification system as appropriate.

TABLE 40

| Prep | | | MS ES+ |
|---|---|---|---|
| # | Chemical Name | Structure | m/z |
| 228 | 2-(5- Fluorobenzo [d][1,3]dioxol- 4-yl)-4,4,5,5- tetramethyl-1,3,2- dioxaborolane | 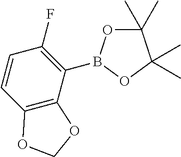 [SMILES: CC1(C)OB(c2c(F)ccc3c2OCO3)OC1(C)C] | 267 [M + H]⁺ |

### Preparation 229

### tert-Butyl (5r,7r)-2-(5-(benzo[c][1,2,5]thiadiazol-4-yl)-1-(4-(ethoxycarbonyl)-2-isopropylphenyl)-1H-pyrazole-3-carboxamido)adamantane-2-carboxylate

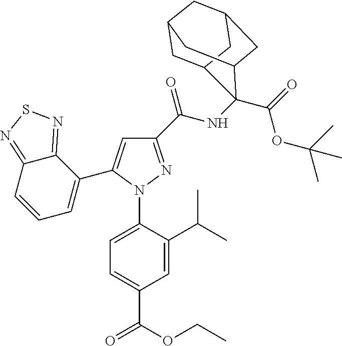 [SMILES: CCOC(=O)c1ccc(-n2nc(C(=O)NC3(C(=O)OC(C)(C)C)C4CC5CC(C4)CC3C5)cc2-c2cccc3nsnc23)c(C(C)C)c1]

A soln of tert-butyl (5r,7r)-2-(5-bromo-1-(4-(ethoxycarbonyl)-2-isopropylphenyl)-1H-pyrazole-3-carboxamido)adamantane-2-carboxylate (75 mg, 0.12 mmol) and 4-(4,4,5,5-Tetramethyl-1,3,2-dioxaborolan-2-yl)benzo[c][1,2,5]thiadiazole (73 mg, 0.37 mmol) in 1,4-Dioxane (3 mL) and H₂O (0.5 mL) was treated with K₂CO₃ (51 mg, 0.37 mmol) and PdCl₂(PPh₃)₂ (21 mg, 0.031 mmol) at RT under N₂. The mixture was degassed with N₂ for 5 min. After stirring at 100° C. for 16 h the mixture was cooled, concentrated, and purified by silica gel column chromatography EA/Hep (0 to 100%) to afford title compound (92 mg, 87%) as a beige solid. MS ES+ m/z 671 [M+H]⁺.

The following compounds were prepared in a manner essentially analogous to the preparation method of tert-Butyl (5r,7r)-2-(5-(benzo[c][1,2,5]thiadiazol-4-yl)-1-(4-(ethoxycarbonyl)-2-isopropylphenyl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 41

| Prep | | | |
|---|---|---|---|
| # | Chemical Name | Structure | MS ES+ m/z |
| 230 | tert-Butyl (5r,7r)-2-(1-(4- (ethoxycarbonyl)-2- isopropylphenyl)-5-(5- (trifluoromethoxy)benzo [d][1,3]dioxol-4-yl)-1H- pyrazole-3- carboxamido)adamantane- 2-carboxylate | 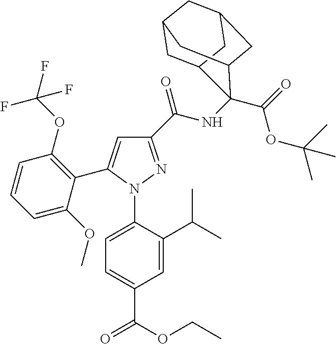 [SMILES: CCOC(=O)c1ccc(-n2nc(C(=O)NC3(C(=O)OC(C)(C)C)C4CC5CC(C4)CC3C5)cc2-c2c(OC)cccc2OC(F)(F)F)c(C(C)C)c1] | 656 [M − tBu + H]⁺ |
| 231 | tert-Butyl (5r,7r)-2-(1-(4- (ethoxycarbonyl)-2- isopropylphenyl)-5-(5- fluorobenzo[d][1,3]dioxol- 4-yl)-1H-pyrazole-3- carboxamido)adamantane- 2-carboxylate | 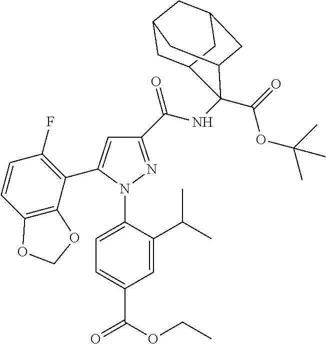 [SMILES: CCOC(=O)c1ccc(-n2nc(C(=O)NC3(C(=O)OC(C)(C)C)C4CC5CC(C4)CC3C5)cc2-c2c(F)ccc3c2OCO3)c(C(C)C)c1] | 618 [M − tBu]⁺ |

### Preparation 232

### tert-Butyl (5r,7r)-2-(1-(2-cyclobutyl-4-((3-(dimethylamino)propyl)(methyl)carbamoyl)phenyl)-5-(2,6-dimethoxyphenyl)-1H-pyrazole-3-carboxamido)adamantane-2-carboxylate

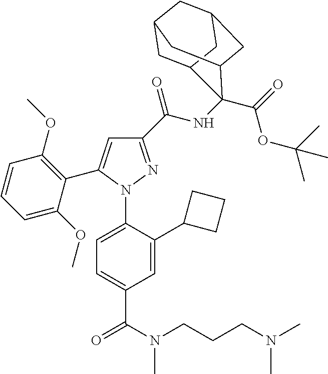 [SMILES: COc1cccc(OC)c1-c1cc(C(=O)NC2(C(=O)OC(C)(C)C)C3CC4CC(C3)CC2C4)nn1-c1ccc(C(=O)N(C)CCCN(C)C)cc1C1CCC1]

A soln of tert-butyl (5r,7r)-2-(1-(2-bromo-4-((3-(dimethylamino)propyl)(methyl)carbamoyl)phenyl)-5-(2,6-dimethoxyphenyl)-1H-pyrazole-3-carboxamido)adamantane-2-carboxylate (70 mg, 0.090 mmol), potassium cyclobutyltrifluoroboranuide (29 mg, 0.18 mmol), Pd(OAc) 2 (4.0 mg, 0.018 mmol), di(adamantan-1-yl)(butyl)phosphine (9.7 mg, 0.027 mmol), and Cs₂CO₃ (88 mg, 0.27 mmol) in toluene (2 mL) and H₂O (0.2 mL) was purged with argon for 2 minutes. After stirring at 110° C. for 16 h, the mixture was dried under a stream of N₂, dissolved in DMF, filtered and purified by reversed-phase flash chromatography with the following conditions: column, C18; mobile phase, 10 to 100% ACN in MeOH (10 mM NH₄HCO₃) to afford title compound (21 mg, 31%) as a white solid. MS ES+ m/z 755 [M+H]⁺.

### Preparation 233

### 4-(5-(Benzo[c][1,2,5]thiadiazol-4-yl)-3-(((5r,7r)-2-(tert-butoxycarbonyl)adamantan-2-yl)carbamoyl)-1H-pyrazol-1-yl)-3-isopropylbenzoic acid

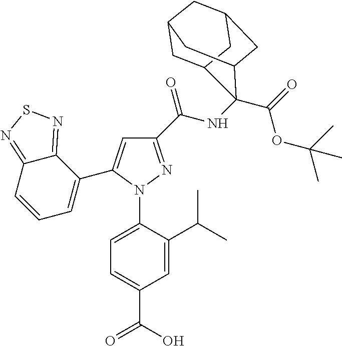 [SMILES: CC(C)c1cc(C(=O)O)ccc1-n1nc(C(=O)NC2(C(=O)OC(C)(C)C)C3CC4CC(C3)CC2C4)cc1-c1cccc2nsnc12]

A soln of tert-butyl (5r,7r)-2-(5-(benzo[c][1,2,5]thiadiazol-4-yl)-1-(4-(ethoxycarbonyl)-2-isopropylphenyl)-1H-pyrazole-3-carboxamido)adamantane-2-carboxylate (26 mg, 39 μmol) in 2-Methyltetrahydrofuran (0.5 mL) and EtOH (0.5 mL), was treated with LiOH (4.6 mg, 0.19 mmol) in H₂O (0.5 mL). After stirring for 3 h at RT the mixture was concentrated to afford crude title compound (25 mg, 100%). MS ES+ m/z 643 [M+H]⁺.

The following compounds were prepared in a manner essentially analogous to the preparation method of 4-(5-(Benzo[c][1,2,5]thiadiazol-4-yl)-3-(((5r,7r)-2-(tert-butoxycarbonyl)adamantan-2-yl)carbamoyl)-1H-pyrazol-1-yl)-3-isopropylbenzoic acid using the appropriate reagents, adjusting temperature, adjusting reaction time to determine completion of the reaction, and adjusting the purification system as appropriate.

TABLE 42

| Prep | | | |
|---|---|---|---|
| # | Chemical Name | Structure | MS ES+ m/z |
| 234 | 4-(3-(((5r,7r)-2-(tert- Butoxycarbonyl)adamantan- 2-yl)carbamoyl)-5-(5- (trifluoromethoxy)benzo[d] [1,3]dioxol-4-yl)-1H- pyrazol-1-yl)-3- isopropylbenzoic acid | 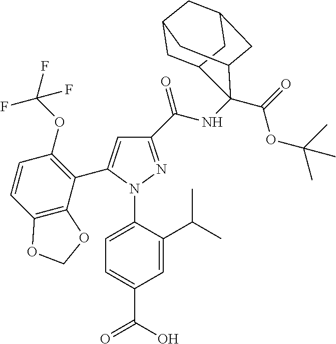 [SMILES: CC(C)c1cc(C(=O)O)ccc1-n1nc(C(=O)NC2(C(=O)OC(C)(C)C)C3CC4CC(C3)CC2C4)cc1-c1c(OC(F)(F)F)ccc2c1OCO2] | 656 [M − tBu + H]⁺ |
| 235 | 4-(3-(((5r,7r)-2-(tert- Butoxycarbonyl)adamantan- 2-yl)carbamoyl)-5-(5- fluorobenzo[d][1,3]dioxol- 4-yl)-1H-pyrazol-1-yl)- 3-isopropylbenzoic acid | 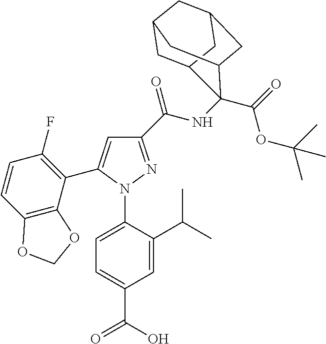 [SMILES: CC(C)c1cc(C(=O)O)ccc1-n1nc(C(=O)NC2(C(=O)OC(C)(C)C)C3CC4CC(C3)CC2C4)cc1-c1c(F)ccc2c1OCO2] | 644 [M − H]⁻ |
| 236 | tert-Butyl (5r,7r)-2-(1-(4- amino-2- isopropylphenyl)-5-(5- (trifluoromethoxy)benzo [d][1,3]dioxol-4-yl)-1H- pyrazole-3- carboxamido)adamantane- 2-carboxylate | 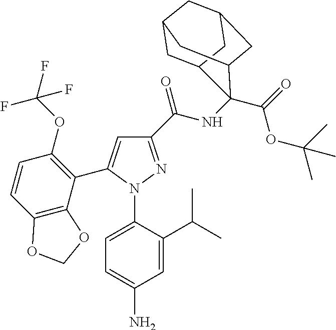 [SMILES: CC(C)c1cc(N)ccc1-n1nc(C(=O)NC2(C(=O)OC(C)(C)C)C3CC4CC(C3)CC2C4)cc1-c1c(OC(F)(F)F)ccc2c1OCO2] | 681 [M − H]⁻ |

### Preparation 46

### tert-Butyl (5r,7r)-2-(1-(2-isopropyl-4-((trimethylsilyl) ethynyl)phenyl)-5-(5-methoxybenzo[d][1,3]dioxol-4-yl)-1H-pyrazole-3-carboxamido)adamantane-2-carboxylate

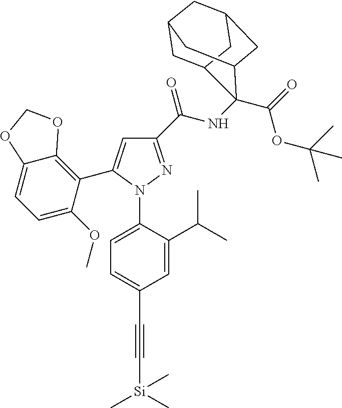 [SMILES: COc1ccc2c(c1-c1cc(C(=O)NC3(C(=O)OC(C)(C)C)C4CC5CC(C4)CC3C5)nn1-c1ccc(C#C[Si](C)(C)C)cc1C(C)C)OCO2]

A soln of tert-butyl (5r,7r)-2-(1-(4-bromo-2-isopropylphenyl)-5-(5-methoxybenzo[d][1,3]dioxol-4-yl)-1H-pyrazole-3-carboxamido)adamantane-2-carboxylate (450 mg, 520 μmol), in Toluene (10 mL) at RT under N₂ was treated with PdCl₂(PPh₃)₂ (18.2 mg, 26.0 μmol), TEA (217 μL, 1.56 mmol) CuI (4.95 mg, 26.0 μmol) and Ethynyl-trimethyl-silane (218 μL, 1.56 mmol). The mixture was degassed with N₂ and stirred at 100° C. for 1 h before being cooled. The mixture was filtered, concentrated, and purified by silica gel column chromatography EA/Hep (0 to 100%) to afford title compound (280 mg, 76%) as a yellow oil. MS ES+ m/z 711 [M+H]⁺.

The following compounds were prepared in a manner essentially analogous to the preparation method of tert-Butyl (5r,7r)-2-(1-(2-isopropyl-4-((trimethylsilyl) ethynyl)phenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 43

| Prep | | | MS ES+ |
|---|---|---|---|
| # | Chemical Name | Structure | m/z |
| 237 | tert-Butyl (5r,7r)-2- (1-(4-(4-hydroxybut- 1-yn-1-yl)-2- isopropylphenyl)-5- (5- methoxybenzo[d][1,3] dioxol-4-yl)-1H- pyrazole-3- carboxamido)adamantane- 2-carboxylate | 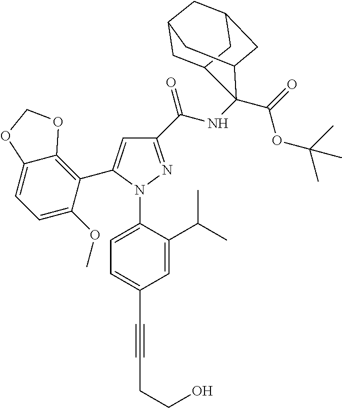 [SMILES: COc1ccc2c(c1-c1cc(C(=O)NC3(C(=O)OC(C)(C)C)C4CC5CC(C4)CC3C5)nn1-c1ccc(C#CCCO)cc1C(C)C)OCO2] | 682 [M + H]⁺ |

### Preparation 395

### tert-Butyl (5r,7r)-2-(1-(2-isopropyl-4-(methoxycarbonyl)phenyl)-5-(5-methoxybenzo[d][1,3]dioxol-4-yl)-1H-pyrazole-3-carboxamido)adamantane-2-carboxylate

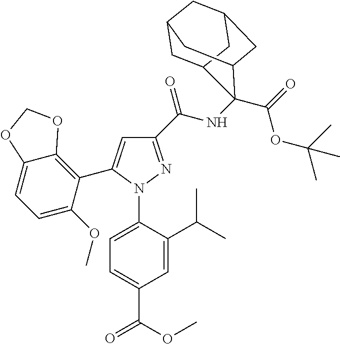 [SMILES: COC(=O)c1ccc(-n2nc(C(=O)NC3(C(=O)OC(C)(C)C)C4CC5CC(C4)CC3C5)cc2-c2c(OC)ccc3c2OCO3)c(C(C)C)c1]

A soln of tert-butyl 2-[1-(4-bromo-2-isopropylphenyl)-5-(5-methoxy-2H-1,3-benzodioxol-4-yl)pyrazole-3-amido]adamantane-2-carboxylate (600 mg, 0.87 mmol) and TEA (175 mg, 1.7 mmol) in MeOH (15 mL) at RT under N₂ was treated with XantPhos (100 mg, 0.17 mmol) and Pd(dppf)Cl₂ (126 mg, 0.17 mmol) via portionwise addition. After stirring at 120° C. ON under CO, the mixture was concentrated and purified by reversed-phase flash chromatography with the following conditions: column, C18; mobile phase, 10% to 100% ACN in H₂O (10 mmol/L NH₄HCO₃) to afford title compound (470 mg, 81%) as an off-white solid. MS ES+ m/z 672 [M+H]⁺.

### Preparation 47

### tert-Butyl (5r,7r)-2-(1-(4-ethynyl-2-isopropylphenyl)-5-(5-methoxybenzo[d][1,3]dioxol-4-yl)-1H-pyrazole-3-carboxamido)adamantane-2-carboxylate

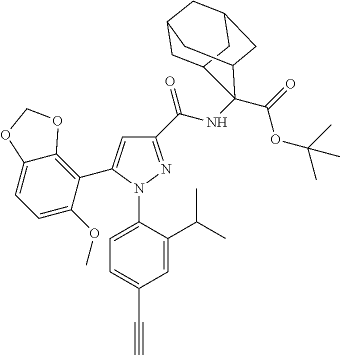 [SMILES: C#Cc1ccc(-n2nc(C(=O)NC3(C(=O)OC(C)(C)C)C4CC5CC(C4)CC3C5)cc2-c2c(OC)ccc3c2OCO3)c(C(C)C)c1]

A soln of tert-butyl (5r,7r)-2-(1-(2-isopropyl-4-((trimethylsilyl) ethynyl)phenyl)-5-(5-methoxybenzo[d][1,3]dioxol-4-yl)-1H-pyrazole-3-carboxamido)adamantane-2-carboxylate (220 mg, 310 μmol) in MeOH (8 mL) was treated with K₂CO₃ (128 mg, 930 μmol). After stirring at RT for 1 h, the mixture was filtered and concentrated to afford title compound (280 mg, 76%). MS ES+ m/z 639 [M+H]⁺.

### Preparation 48

### tert-Butyl (5r,7r)-2-(1-(4-(1-(3-((3-((tert-butoxycarbonyl)(methyl)amino)propyl)(methyl)amino)propyl)-1H-1,2,3-triazol-4-yl)-2-isopropylphenyl)-5-(5-methoxybenzo[d][1,3]dioxol-4-yl)-1H-pyrazole-3-carboxamido)adamantane-2-carboxylate

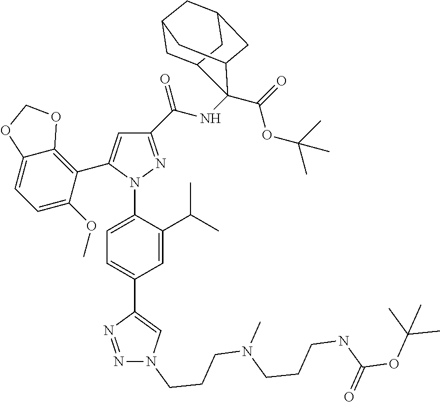

A soln of tert-butyl (5r,7r)-2-(1-(4-ethynyl-2-isopropylphenyl)-5-(5-methoxybenzo[d][1,3]dioxol-4-yl)-1H-pyrazole-3-carboxamido)adamantane-2-carboxylate (120 mg, 188 μmol), tert-butyl (3-((3-azidopropyl)(methyl)amino)propyl)(methyl)carbamate (53.7 mg, 188 μmol) in MeOH (4 mL), DCM (2 mL), and H₂O (2 mL) was treated with Sodium 2-(1,2-dihydroxy-ethyl)-4-hydroxy-5-oxo-2,5-dihydro-furan-3-olate (37.3 mg, 188 μmol), and CuSO₄·5H₂O (47.0 mg, 188 μmol). After stirring at RT for 1 h, the mixture was concentrated and purified by silica gel column chromatography EA/Hep (0 to 100%) to afford title compound (139 mg, 80%) as a white solid. MS ES+ m/z 924 [M+H]⁺.

The following compounds were prepared in a manner essentially analogous to the preparation method of tert-Butyl (5r,7r)-2-(1-(4-(1-(3-((3-((tert-butoxycarbonyl)(methyl)amino)propyl)(methyl)amino)propyl)-1H-1,2,3-triazol-4-yl)-2-isopropylphenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 10

| Prep | | | MS ES+ |
|---|---|---|---|
| # | Chemical Name | Structure | m/z |
| 49 | tert-Butyl (5r,7r)-2- (1-(4-(1-(3- (dimethylamino) propyl)- 1H-1,2,3-triazol- 4-yl)-2- isopropylphenyl)-5-(5- methoxybenzo[d][1,3] dioxol-4-yl)-1H- pyrazole-3- carboxamido) adamantane- 2-carboxylate | 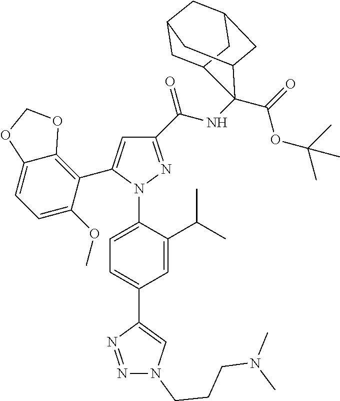 [SMILES: COc1ccc2c(c1-c1cc(C(=O)NC3(C(=O)OC(C)(C)C)C4CC5CC(C4)CC3C5)nn1-c1ccc(-c3cn(CCCN(C)C)nn3)cc1C(C)C)OCO2] | 710 [M − tBu]⁺ |
| 238 | tert-Butyl (5r,7r)-2- (1-(4-(1-(3-((3-((tert- butoxycarbonyl) amino)propyl) (methyl)amino) propyl)-1H-1,2,3- triazol-4-yl)-2- isopropylphenyl)-5- (5- methoxybenzo [d][1,3] dioxol-4-yl)-1H- pyrazole-3- carboxamido) adamantane- 2-carboxylate | 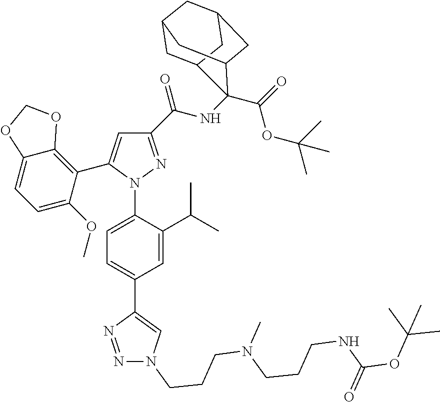 | 810 [M − tBu]⁻ |
| 239 | tert-Butyl (5r,7r)-2- (1-(4-(1-(3-(3-(1,3- dioxoisoindolin-2- yl)propoxy)propyl)- 1H-1,2,3-triazol-4- yl)-2- isopropylphenyl)-5-(5- methoxybenzo[d][1,3] dioxol-4-yl)-1H- pyrazole-3- carboxamido) adamantane-2- carboxylate | 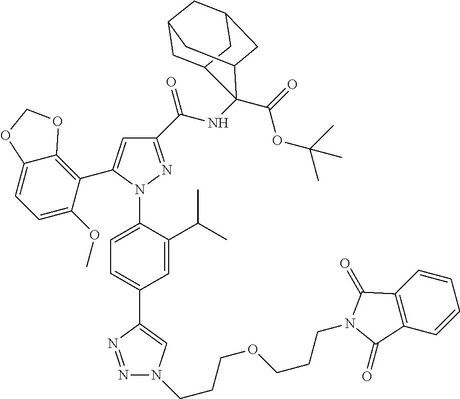 [SMILES: COc1ccc2c(c1-c1cc(C(=O)NC3(C(=O)OC(C)(C)C)C4CC5CC(C4)CC3C5)nn1-c1ccc(-c3cn(CCCOCCCN4C(=O)c5ccccc5C4=O)nn3)cc1C(C)C)OCO2] | 870 [M − tBu]- |

### Preparation 396

### tert-Butyl (5r,7r)-2-(1-(4-(hydrazinecarbonyl)-2-isopropylphenyl)-5-(5-methoxybenzo[d][1,3]dioxol-4-yl)-1H-pyrazole-3-carboxamido)adamantane-2-carboxylate

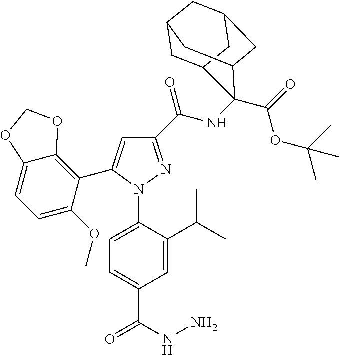 [SMILES: COc1ccc2c(c1-c1cc(C(=O)NC3(C(=O)OC(C)(C)C)C4CC5CC(C4)CC3C5)nn1-c1ccc(C(=O)NN)cc1C(C)C)OCO2]

A soln of tert-butyl 2-{1-[2-isopropyl-4-(methoxycarbonyl)phenyl]-5-(5-methoxy-2H-1,3-benzodioxol-4-yl)pyrazole-3-amido}adamantane-2-carboxylate (460 mg, 0.685 mmol) in EtOH (5 mL) at RT under N₂ was treated with N₂H₄·H₂O (343 mg, 6.85 mmol) via dropwise addition. After stirring at 80° C. ON, the reaction was cooled to RT and quenched with H₂O (15 mL). The aq layer was extracted with EtOAc (3×20 mL), and the combined organic layers were concentrated to afford title compound (450 mg, 100%) as a yellow solid. MS ES+ m/z 672 [M+H]⁺.

### Preparation 397

### tert-Butyl (5r,7r)-2-(1-(4-(2-(4-chlorobutanoyl)hydrazine-1-carbonyl)-2-isopropylphenyl)-5-(5-methoxybenzo[d][1,3]dioxol-4-yl)-1H-pyrazole-3-carboxamido)adamantane-2-carboxylate

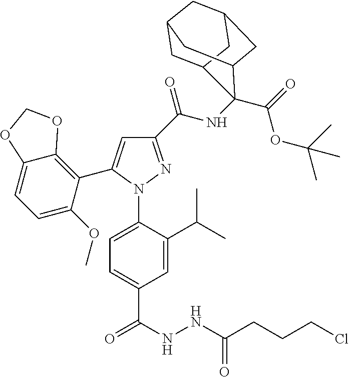 [SMILES: COc1ccc2c(c1-c1cc(C(=O)NC3(C(=O)OC(C)(C)C)C4CC5CC(C4)CC3C5)nn1-c1ccc(C(=O)NNC(=O)CCCCl)cc1C(C)C)OCO2]

A soln of tert-butyl 2-{1-[4-(hydrazinecarbonyl)-2-isopropylphenyl]-5-(5-methoxy-2H-1,3-benzodioxol-4-yl)pyrazole-3-amido}adamantane-2-carboxylate (450 mg, 0.67 mmol) in THF (5 mL) at 0° C. under N₂ was treated with 4-chloro-butanoyl chloride (142 mg, 1.0 mmol) via dropwise addition. After stirring at RT for ON, the mixture was cooled to 0° C. and quenched with ice H₂O. The resulting mixture was extracted with CH₂Cl₂ (3×50 mL). The combined organic layers were washed with brine (1×50 mL), dried over Na₂SO₄, filtered, and concentrated to afford title compound (450 mg, 86.5%) as a yellow solid. MS ES+ m/z 776 [M+H]⁺.

### Preparation 405

### tert-Butyl (5r,7r)-2-(1-(4-(5-(3-chloropropyl)-1,3,4-oxadiazol-2-yl)-2-isopropylphenyl)-5-(5-methoxybenzo[d][1,3]dioxol-4-yl)-1H-pyrazole-3-carboxamido)adamantane-2-carboxylate

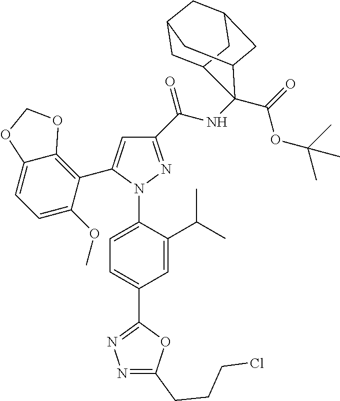 [SMILES: COc1ccc2c(c1-c1cc(C(=O)NC3(C(=O)OC(C)(C)C)C4CC5CC(C4)CC3C5)nn1-c1ccc(-c3nnc(CCCCl)o3)cc1C(C)C)OCO2]

A soln of tert-butyl 2-(1-{4-[N′-(4-chlorobutanoyl)hydrazinecarbonyl]-2-isopropylphenyl}-5-(5-methoxy-2H-1,3-benzodioxol-4-yl)pyrazole-3-amido)adamantane-2-carboxylate (400 mg, 0.515 mmol) in Dioxane (5 mL) at RT was treated with Burgess reagent (368 mg, 1.545 mmol) in portions. After stirring at 120° C. ON under N₂, the mixture was concentrated, dissolved in ACN (4 mL) and purified by reversed-phase flash chromatography with the following conditions: column, C18; mobile phase, 85% to 90% ACN in H₂O (10 mmol/L NH₄HCO₃) to afford title compound (310 mg, 100%) as an off-white solid. MS ES+ m/z 758 [M+H]⁺.

### Preparation 50

### tert-Butyl (5r,7r)-2-(1-(2-isopropyl-4-(2-(1-methylpyrrolidin-3-yl)acetamido) phenyl)-5-(5-methoxybenzo[d][1,3]dioxol-4-yl)-1H-pyrazole-3-carboxamido)adamantane-2-carboxylate

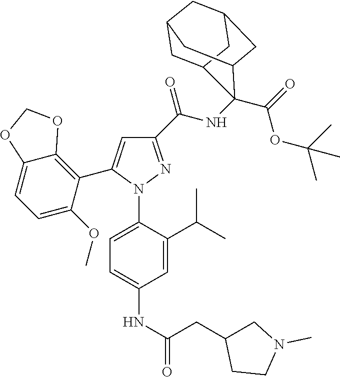 [SMILES: COc1ccc2c(c1-c1cc(C(=O)NC3(C(=O)OC(C)(C)C)C4CC5CC(C4)CC3C5)nn1-c1ccc(NC(=O)CC3CCN(C)C3)cc1C(C)C)OCO2]

A soln of tert-butyl (5r,7r)-2-(1-(4-amino-2-isopropylphenyl)-5-(5-methoxybenzo[d][1,3]dioxol-4-yl)-1H-pyrazole-3-carboxamido)adamantane-2-carboxylate (30 mg, 48 μmol), DMF (0.5 mL), 2-(1-methylpyrrolidin-3-yl)acetic acid (14 mg, 95 μmol), DIEA (41 μL, 0.24 mmol) and T3P (84 μL, 50% Wt, 0.14 mmol) was stirred at 22° C. for ON. The mixture was concentrated to afford crude title compound (36 mg, 100%). MS ES+ m/z 755 [M+H]⁺.

The following compounds were prepared in a manner essentially analogous to the preparation method of tert-Butyl (5r,7r)-2-(1-(2-isopropyl-4-(2-(1-methylpyrrolidin-3-yl)acetamido) phenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 11

| Prep | | | |
|---|---|---|---|
| # | Chemical Name | Structure | MS ES+ m/z |
| 51 | tert-Butyl (5r,7r)-2-(1-(2- isopropyl-4-(2-(1- methylpiperidin-4- yl)acetamido)phenyl)-5- (5-methoxybenzo[d][1,3] dioxol-4-yl)-1H-pyrazole-3- carboxamido)adamantane- 2-carboxylate | 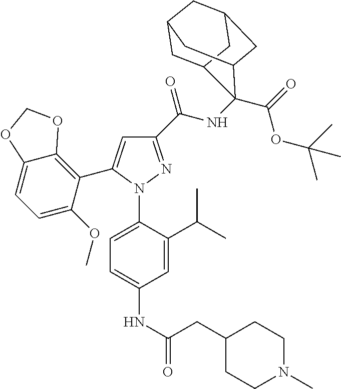 [SMILES: COc1ccc2c(c1-c1cc(C(=O)NC3(C(=O)OC(C)(C)C)C4CC5CC(C4)CC3C5)nn1-c1ccc(NC(=O)CC3CCN(C)CC3)cc1C(C)C)OCO2] | 769 [M + H]⁺ |
| 52 | tert-Butyl (5r,7r)-2-(1-(4- (4- (dimethylamino)butanamido)- 2-isopropylphenyl)- 5-(5- methoxybenzo[d][1,3] dioxol-4-yl)-1H-pyrazole-3- carboxamido)adamantane- 2-carboxylate | 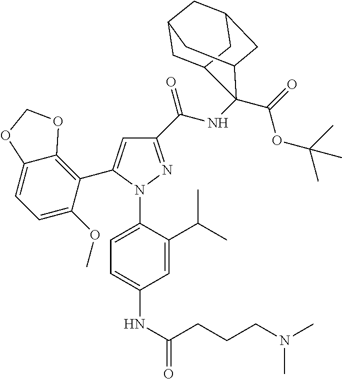 [SMILES: COc1ccc2c(c1-c1cc(C(=O)NC3(C(=O)OC(C)(C)C)C4CC5CC(C4)CC3C5)nn1-c1ccc(NC(=O)CCCN(C)C)cc1C(C)C)OCO2] | 743 [M + H]⁺ |
| 53 | tert-Butyl (5r,7r)-2-(1- (4′-(4- (dimethylamino)butanamido)- 3-isopropyl-[1,1′- biphenyl]-4-yl)-5-(5- methoxybenzo[d][1,3] dioxol-4-yl)-1H-pyrazole-3- carboxamido)adamantane- 2-carboxylate | 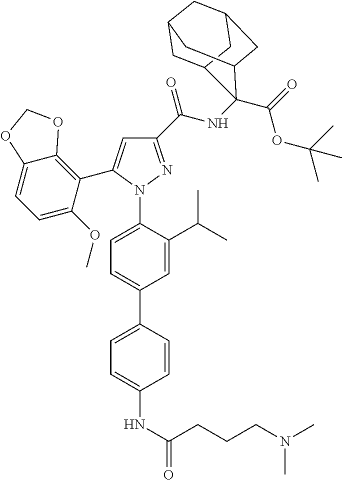 [SMILES: COc1ccc2c(c1-c1cc(C(=O)NC3(C(=O)OC(C)(C)C)C4CC5CC(C4)CC3C5)nn1-c1ccc(-c3ccc(NC(=O)CCCN(C)C)cc3)cc1C(C)C)OCO2] | 382 [M − tBu⁺2H]/2⁺ |
| 54 | tert-Butyl (5r,7r)-2-(1- (3′-(4- (dimethylamino)butanamido)- 3-isopropyl-[1,1′- biphenyl]-4-yl)-5-(5- methoxybenzo[d][1,3] dioxol-4-yl)-1H-pyrazole-3- carboxamido)adamantane- 2-carboxylate | 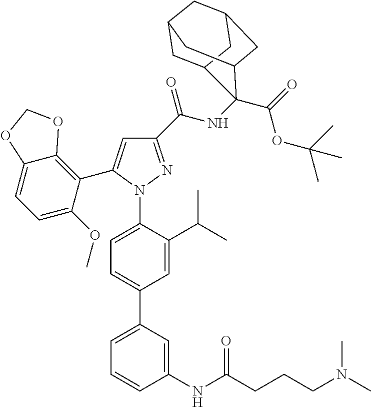 [SMILES: COc1ccc2c(c1-c1cc(C(=O)NC3(C(=O)OC(C)(C)C)C4CC5CC(C4)CC3C5)nn1-c1ccc(-c3cccc(NC(=O)CCCN(C)C)c3)cc1C(C)C)OCO2] | 819 [M + H]⁺ |
| 55 | tert-Butyl (5r,7r)-2-(1- (4′-(4-(dimethylamino)- N-methylbutanamido)-3- isopropyl-[1,1′- biphenyl]-4-yl)-5-(5- methoxybenzo[d][1,3] dioxol-4-yl)-1H-pyrazole-3- carboxamido)adamantane- 2-carboxylate | 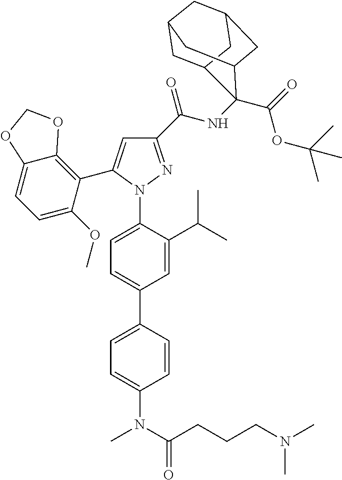 [SMILES: COc1ccc2c(c1-c1cc(C(=O)NC3(C(=O)OC(C)(C)C)C4CC5CC(C4)CC3C5)nn1-c1ccc(-c3ccc(N(C)C(=O)CCCN(C)C)cc3)cc1C(C)C)OCO2] | 833 [M + H]⁺ |
| 240 | tert-butyl (5r,7r)-2-(1-(2- isopropyl-4-(1- methylpiperidine-4- carboxamido)phenyl)-5- (5-methoxybenzo[d][1,3] dioxol-4-yl)-1H-pyrazole-3- carboxamido)adamantane- 2-carboxylate | 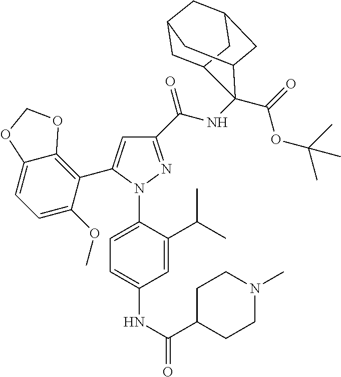 [SMILES: COc1ccc2c(c1-c1cc(C(=O)NC3(C(=O)OC(C)(C)C)C4CC5CC(C4)CC3C5)nn1-c1ccc(NC(=O)C3CCN(C)CC3)cc1C(C)C)OCO2] | 752 [M + H]⁺ |
| 241 | tert-Butyl (5r,7r)-2-(1-(2- isopropyl-4-(3-(1- methylpiperidin-4- yl)propanamido)phenyl)- 5-(5- methoxybenzo[d][1,3] dioxol-4-yl)-1H-pyrazole-3- carboxamido)adamantane- 2-carboxylate | 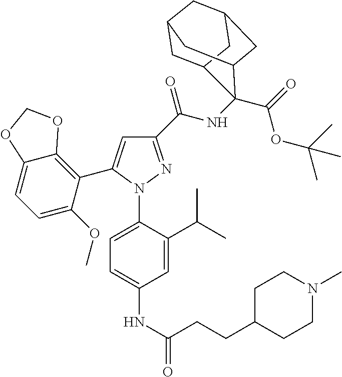 [SMILES: COc1ccc2c(c1-c1cc(C(=O)NC3(C(=O)OC(C)(C)C)C4CC5CC(C4)CC3C5)nn1-c1ccc(NC(=O)CCC3CCN(C)CC3)cc1C(C)C)OCO2] | 783 [M + H]⁺ |
| 242 | tert-Butyl (5r,7r)-2-(1-(4- (2-(1-(3- (((benzyloxy)carbonyl) amino)propyl)piperidin-4- yl)acetamido)-2- isopropylphenyl)-5-(5- methoxybenzo [d][1,3]dioxol- 4-yl)-1H-pyrazole-3- carboxamido)adamantane- 2-carboxylate | 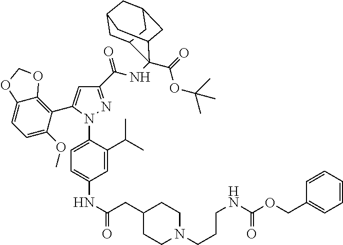 [SMILES: COc1ccc2c(c1-c1cc(C(=O)NC3(C(=O)OC(C)(C)C)C4CC5CC(C4)CC3C5)nn1-c1ccc(NC(=O)CC3CCN(CCCNC(=O)OCc4ccccc4)CC3)cc1C(C)C)OCO2] | 946 [M + H]⁺ |
| 243 | tert-Butyl (5r,7r)-2-(1-(4- (5-(4- (dimethylamino)butanamido) pyridin-2-yl)-2- isopropylphenyl)-5-(5- methoxybenzo[d][1,3] dioxol-4-yl)-1H-pyrazole-3- carboxamido)adamantane- 2-carboxylate | 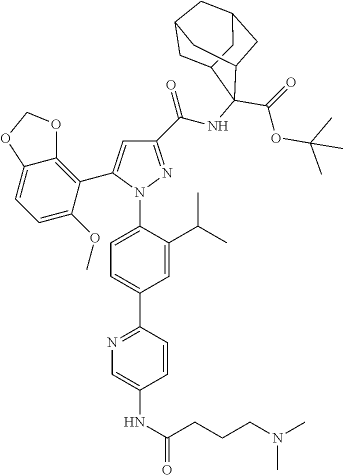 [SMILES: COc1ccc2c(c1-c1cc(C(=O)NC3(C(=O)OC(C)(C)C)C4CC5CC(C4)CC3C5)nn1-c1ccc(-c3ccc(NC(=O)CCCN(C)C)cn3)cc1C(C)C)OCO2] | 820 [M + H]⁺ |
| 244 | tert-Butyl (5r,7r)-2-(1-(4- (6-(4- (dimethylamino)butanamido) pyridazin-3-yl)-2- isopropylphenyl)-5-(5- methoxybenzo[d][1,3] dioxol-4-yl)-1H-pyrazole-3- carboxamido)adamantane- 2-carboxylate | 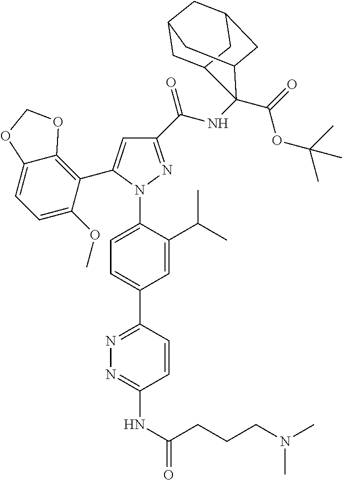 [SMILES: COc1ccc2c(c1-c1cc(C(=O)NC3(C(=O)OC(C)(C)C)C4CC5CC(C4)CC3C5)nn1-c1ccc(-c3ccc(NC(=O)CCCN(C)C)nn3)cc1C(C)C)OCO2] | 821 [M + H]⁺ |
| 245 | tert-Butyl (5r,7r)-2-(1-(4-(4- (dimethylamino)butanamido)- 2-isopropylphenyl)-5-(5- (trifluoromethoxy)benzo [d][1,3]dioxol-4-yl)-1H- pyrazole-3- carboxamido)adamantane- 2-carboxylate | 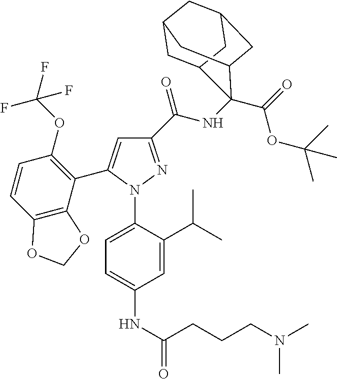 [SMILES: CC(C)c1cc(NC(=O)CCCN(C)C)ccc1-n1nc(C(=O)NC2(C(=O)OC(C)(C)C)C3CC4CC(C3)CC2C4)cc1-c1c(OC(F)(F)F)ccc2c1OCO2] | 794 [M − H]⁻ |
| 246 | tert-Butyl (5r,7r)-2-(1-(4- (4-((S)-6-((((9H-fluoren- 9-yl)methoxy)carbonyl) amino)-2-(4-((4-(4- iodophenyl)butanamido) methyl)benzamido) hexanamido)butanamido)-2- isopropylphenyl)-5-(5- methoxybenzo[d][1,3] dioxol-4-yl)-1H-pyrazole-3- carboxamido) adamantane-2-carboxylate | 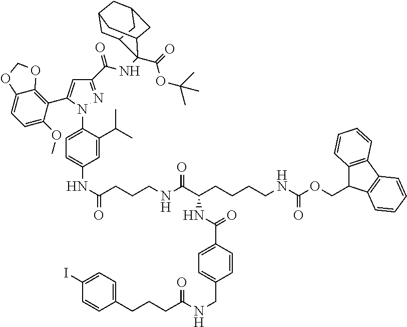 [SMILES: COc1ccc2c(c1-c1cc(C(=O)NC3(C(=O)OC(C)(C)C)C4CC5CC(C4)CC3C5)nn1-c1ccc(NC(=O)CCCNC(=O)[C@H](CCCCNC(=O)OCC3c4ccccc4-c4ccccc43)NC(=O)c3ccc(CNC(=O)CCCc4ccc(I)cc4)cc3)cc1C(C)C)OCO2] | 1470 [M + H]⁺ |
| 336 | tert-Butyl (5r,7r)-2-(1-(4- (2-(1-((S)-5-(tert- butoxy)-2-((tert- butoxycarbonyl)amino)-5- oxopentanoyl)piperidin- 4-yl)ethoxy)-2- isopropylphenyl)-5-(5- methoxybenzo[d][1,3] dioxol-4-yl)-1H-pyrazole-3- carboxamido)adamantane- 2-carboxylate | 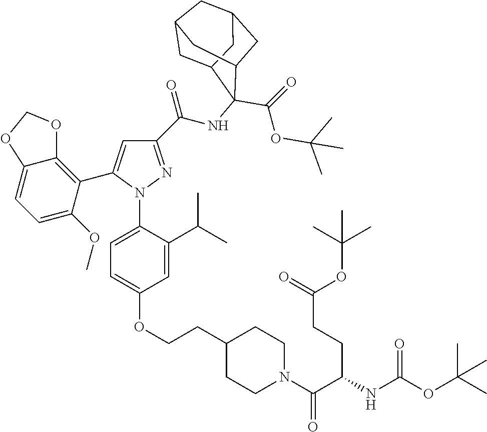 [SMILES: COc1ccc2c(c1-c1cc(C(=O)NC3(C(=O)OC(C)(C)C)C4CC5CC(C4)CC3C5)nn1-c1ccc(OCCC3CCN(C(=O)[C@H](CCC(=O)OC(C)(C)C)NC(=O)OC(C)(C)C)CC3)cc1C(C)C)OCO2] | 1027 [M + H]⁺ |

### Preparation 247

### tert-Butyl (5r,7r)-2-(1-(4-(2-(1-(3-aminopropyl)piperidin-4-yl)acetamido)-2-isopropylphenyl)-5-(5-methoxybenzo[d][1,3]dioxol-4-yl)-1H-pyrazole-3-carboxamido)adamantane-2-carboxylate

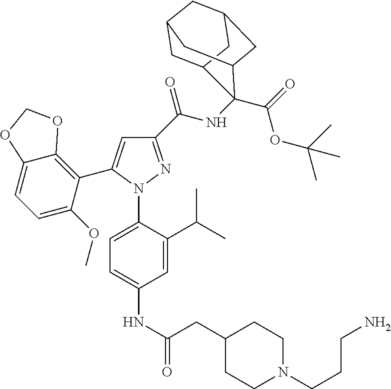 [SMILES: COc1ccc2c(c1-c1cc(C(=O)NC3(C(=O)OC(C)(C)C)C4CC5CC(C4)CC3C5)nn1-c1ccc(NC(=O)CC3CCN(CCCN)CC3)cc1C(C)C)OCO2]

A soln of tert-butyl (5r,7r)-2-(1-(4-(2-(1-(3-(((benzyloxy)carbonyl)amino)propyl)piperidin-4-yl)acetamido)-2-isopropylphenyl)-5-(5-methoxybenzo[d][1,3]dioxol-4-yl)-1H-pyrazole-3-carboxamido)adamantane-2-carboxylate (78 mg, 83 μmol) in EtOH (1.5 mL) was treated with 5 wt. % Pd(OH) 2/C (35 mg, 17 μmol)). After stirring at RT for ON under H₂, the mixture was filtered through diatomaceous earth. The filtrate was concentrated to afford crude title compound (67 mg, >100%) as an off-white solid. MS ES+ m/z 812 [M+H]⁺.

### Preparation 56

### tert-Butyl (5r,7r)-2-(1-(4-((3-(dimethylamino)propyl)(methyl)carbamoyl)-2-isopropylphenyl)-5-(5-methoxybenzo[d][1,3]dioxol-4-yl)-1H-pyrazole-3-carboxamido)adamantane-2-carboxylate

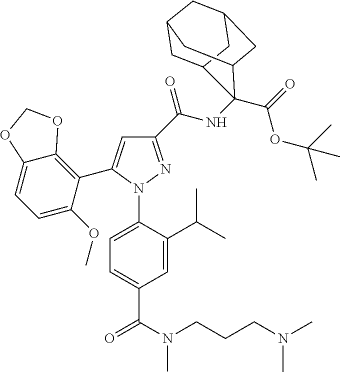 [SMILES: COc1ccc2c(c1-c1cc(C(=O)NC3(C(=O)OC(C)(C)C)C4CC5CC(C4)CC3C5)nn1-c1ccc(C(=O)N(C)CCCN(C)C)cc1C(C)C)OCO2]

A soln of 1-(4-((3-(dimethylamino)propyl)(methyl)carbamoyl)-2-isopropylphenyl)-5-(5-methoxybenzo[d][1,3]dioxol-4-yl)-1H-pyrazole-3-carboxylic acid (70 mg, 0.13 mmol) in DMF (2 mL), tert-butyl (5r,7r)-2-aminoadamantane-2-carboxylate (40 mg, 0.16 mmol) at 0° C. was treated with DIEA (70 μL, 0.40 mmol) and T3P (0.12 mL, 0.40 mmol). After stirring at RT for 1 h the mixture was diluted with water, and extracted with EA (3×10 mL). The combined organic layers were washed with sat. NaHCO₃ solution, brine, dried over Na₂SO₄, filtered, and concentrated to afford crude title compound (50 mg, 49%) as a brown oil. MS ES+ m/z 756 [M+H]⁺.

The following compounds were prepared in a manner essentially analogous to the preparation method of tert-Butyl (5r,7r)-2-(1-(4-((3-(dimethylamino)propyl)(methyl)carbamoyl)-2-isopropylphenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 12

| | | | MS |
|---|---|---|---|
| Prep | | | ES⁺ |
| # | Chemical Name | Structure | m/z |
| 57 | tert-Butyl (5r,7r)-2-(1- (3′-((3- (dimethylamino)propyl) (methyl)carbamoyl)-3- isopropyl-[1,1′- biphenyl]-4-yl)-5-(5- methoxybenzo[d][1,3] dioxol-4-yl)-1H-pyrazole-3- carboxamido)adamantane- 2-carboxylate | 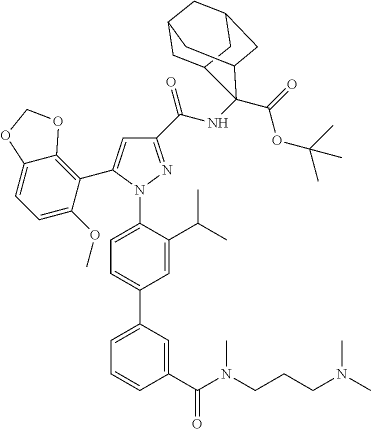 [SMILES: COc1ccc2c(c1-c1cc(C(=O)NC3(C(=O)OC(C)(C)C)C4CC5CC(C4)CC3C5)nn1-c1ccc(-c3cccc(C(=O)N(C)CCCN(C)C)c3)cc1C(C)C)OCO2] | 833 [M + H]⁺ |
| 58 | tert-Butyl (5r,7r)-2-(1- (4′-((3- (dimethylamino)propyl) (methyl)carbamoyl)-3- isopropyl-[1,1′- biphenyl]-4-yl)-5-(5- methoxybenzo[d][1,3] dioxol-4-yl)-1H-pyrazole-3- carboxamido)adamantane- 2-carboxylate | 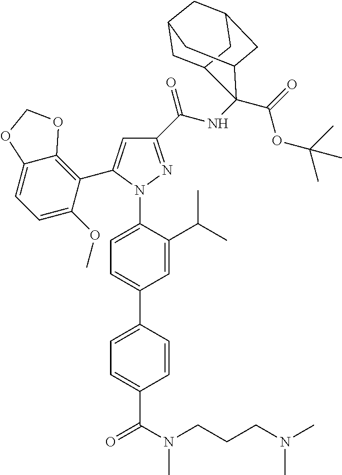 [SMILES: COc1ccc2c(c1-c1cc(C(=O)NC3(C(=O)OC(C)(C)C)C4CC5CC(C4)CC3C5)nn1-c1ccc(-c3ccc(C(=O)N(C)CCCN(C)C)cc3)cc1C(C)C)OCO2] | 389 [M − tBu + 2H] 1/2⁺ |
| 248 | tert-Butyl (5r,7r)-2-(5- (benzo[c][1,2,5]thiadiazo 1-4-yl)-1-(4-((3- (dimethylamino)propyl) (methyl)carbamoyl)-2- isopropylphenyl)-1H- pyrazole-3- carboxamido)adamantane- 2-carboxylate | 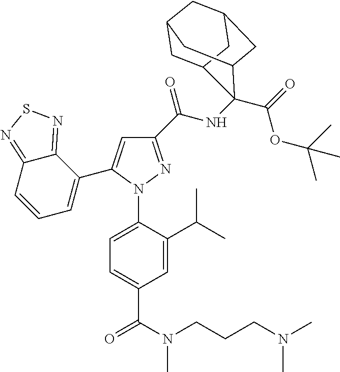 [SMILES: CC(C)c1cc(C(=O)N(C)CCCN(C)C)ccc1-n1nc(C(=O)NC2(C(=O)OC(C)(C)C)C3CC4CC(C3)CC2C4)cc1-c1cccc2nsnc12] | 740 [M + H]⁺ |
| 249 | tert-Butyl (5r,7r)-2-(1-(4- (6-((3- (dimethylamino)propyl) (methyl)carbamoyl)pyridin- 3-yl)-2- isopropylphenyl)-5-(5- methoxybenzo[d][1,3]dioxol- 4-yl)-1H-pyrazole-3- carboxamido)adamantane- 2-carboxylate | 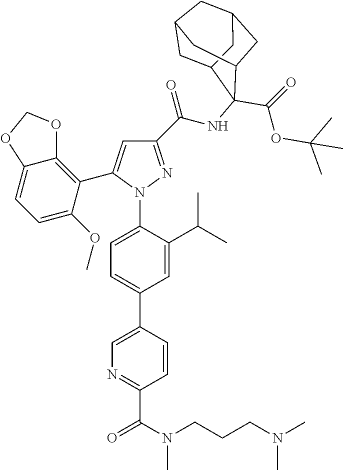 [SMILES: COc1ccc2c(c1-c1cc(C(=O)NC3(C(=O)OC(C)(C)C)C4CC5CC(C4)CC3C5)nn1-c1ccc(-c3ccc(C(=O)N(C)CCCN(C)C)nc3)cc1C(C)C)OCO2] | 834 [M + H]⁺ |
| 250 | tri-tert-Butyl 2,2′,2″-(10- (2-((3-((3-(5-(4-(3- (((5r,7r)-2-(tert- butoxycarbonyl) adamantan- 2-yl)carbamoyl)-5-(5- methoxybenzo[d][1,3] dioxol-4-yl)-1H-pyrazol- 1-yl)-3- isopropylphenyl) picolinamido) propyl)(methyl) amino)propyl)amino)-2- oxoethyl)-1,4,7,10- tetraazacyclododecane- 1,4,7-triyl)triacetate | 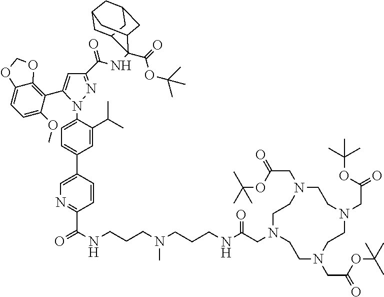 [SMILES: COc1ccc2c(c1-c1cc(C(=O)NC3(C(=O)OC(C)(C)C)C4CC5CC(C4)CC3C5)nn1-c1ccc(-c3ccc(C(=O)NCCCN(C)CCCNC(=O)CN4CCN(CC(=O)OC(C)(C)C)CCN(CC(=O)OC(C)(C)C)CCN(CC(=O)OC(C)(C)C)CC4)nc3)cc1C(C)C)OCO2] | 1417 [M + H]⁺ |
| 251 | tert-Butyl (5r,7r)-2-(1-(4- ((3- (dimethylamino)propyl) (methyl)carbamoyl)-2- isopropylphenyl)-5-(5- (trifluoromethoxy)benzo [d][1,3]dioxol-4-yl)-1H- pyrazole-3- carboxamido)adamantane- 2-carboxylate | 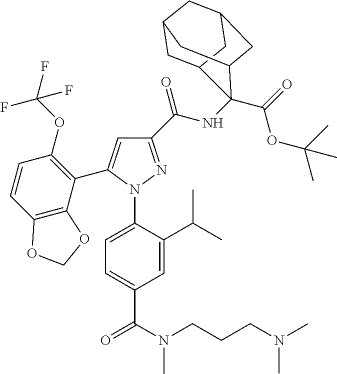 [SMILES: CC(C)c1cc(C(=O)N(C)CCCN(C)C)ccc1-n1nc(C(=O)NC2(C(=O)OC(C)(C)C)C3CC4CC(C3)CC2C4)cc1-c1c(OC(F)(F)F)ccc2c1OCO2] | 811 [M + H]⁺ |
| 252 | tert-Butyl (5r,7r)-2-(1-(4- ((3- (dimethylamino)propyl) (methyl)carbamoyl)-2- isopropylphenyl)-5-(5- fluorobenzo[d][1,3]dioxol- 4-yl)-1H-pyrazole-3- carboxamido)adamantane- 2-carboxylate | 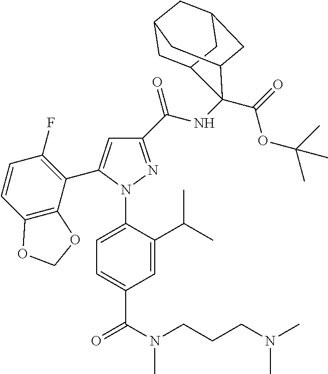 [SMILES: CC(C)c1cc(C(=O)N(C)CCCN(C)C)ccc1-n1nc(C(=O)NC2(C(=O)OC(C)(C)C)C3CC4CC(C3)CC2C4)cc1-c1c(F)ccc2c1OCO2] | 745 [M + H]⁺ |
| 253 | tri-tert-Butyl 2,2′,2″-(10- (2-((3-((3-(3-(4-(3- (((5r,7r)-2-(tert- butoxycarbonyl)adamantan- 2-yl)carbamoyl)-5-(5- methoxybenzo[d][1,3] dioxol-4-yl)-1H-pyrazol-1- yl)-3- isopropylphenyl)quinoline- 7- carboxamido)propyl) (methyl)amino)propyl)amino)- 2-oxoethyl)-1,4,7,10- tetraazacyclododecane- 1,4,7-triyl)triacetate | 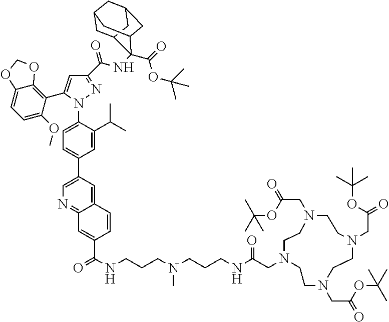 [SMILES: COc1ccc2c(c1-c1cc(C(=O)NC3(C(=O)OC(C)(C)C)C4CC5CC(C4)CC3C5)nn1-c1ccc(-c3cnc4cc(C(=O)NCCCN(C)CCCNC(=O)CN5CCN(CC(=O)OC(C)(C)C)CCN(CC(=O)OC(C)(C)C)CCN(CC(=O)OC(C)(C)C)CC5)ccc4c3)cc1C(C)C)OCO2] | 1467 [M + H]⁺ |
| 337 | tri-tert-Butyl 2,2′,2″-(10- (2-((3-((3-(6-(4-(3- (((5r, 7r)-2-(tert- butoxycarbonyl)adamantan- 2-yl)carbamoyl)-5-(5- methoxybenzo[d][1,3]dioxol- 4-yl)-1H-pyrazol-1- yl)-3- isopropylphenyl)quinoxaline- 2- carboxamido)propyl)(methyl) amino)propyl)amino)- 2-oxoethyl)-1,4,7,10- tetraazacyclododecane- 1,4,7-triyl)triacetate | 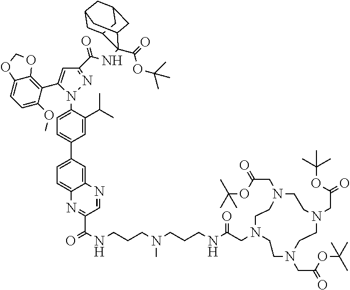 [SMILES: COc1ccc2c(c1-c1cc(C(=O)NC3(C(=O)OC(C)(C)C)C4CC5CC(C4)CC3C5)nn1-c1ccc(-c3ccc4nc(C(=O)NCCCN(C)CCCNC(=O)CN5CCN(CC(=O)OC(C)(C)C)CCN(CC(=O)OC(C)(C)C)CCN(CC(=O)OC(C)(C)C)CC5)cnc4c3)cc1C(C)C)OCO2] | 1468 [M + H]⁺ |
| 338 | tri-tert-Butyl 2,2′,2″-(10- (2-((3-((3-(7-(4-(3- (((5r,7r)-2-(tert- butoxycarbonyl)adamantan- 2-yl)carbamoyl)-5-(5- methoxybenzo[d][1,3] dioxol-4-yl)-1H-pyrazol-1- yl)-3-isopropylphenyl) quinoline-3- carboxamido)propyl) (methyl)amino) propyl)amino)- 2-oxoethyl)-1,4,7,10- tetraazacyclododecane- 1,4,7-triyl)triacetate | 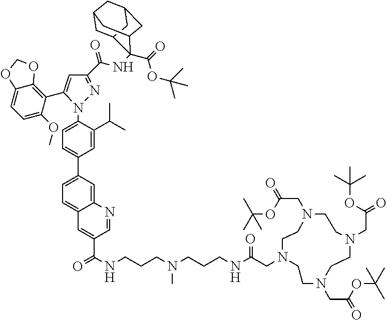 [SMILES: COc1ccc2c(c1-c1cc(C(=O)NC3(C(=O)OC(C)(C)C)C4CC5CC(C4)CC3C5)nn1-c1ccc(-c3ccc4cc(C(=O)NCCCN(C)CCCNC(=O)CN5CCN(CC(=O)OC(C)(C)C)CCN(CC(=O)OC(C)(C)C)CCN(CC(=O)OC(C)(C)C)CC5)cnc4c3)cc1C(C)C)OCO2] | 1467 [M + H]⁺ |
| 339 | tri-tert-Butyl 2,2′,2″-(10- (2-((3-((3-(7-(4-(3- (((5r,7r)-2-(tert- butoxycarbonyl)adamantan- 2-yl)carbamoyl)-5-(5- methoxybenzo[d][1,3] dioxol-4-yl)-1H-pyrazol-1- yl)-3-isopropylphenyl)- 1,5-naphthyridine-3- carboxamido)propyl) (methyl)amino)propyl)amino)- 2-oxoethyl)-1,4,7,10- tetraazacyclododecane- 1,4,7-triyl)triacetate | 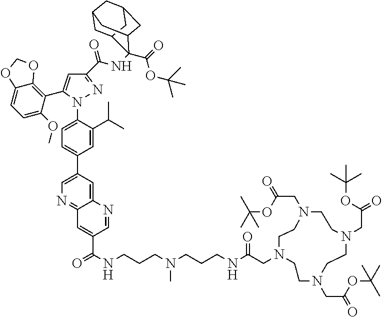 [SMILES: COc1ccc2c(c1-c1cc(C(=O)NC3(C(=O)OC(C)(C)C)C4CC5CC(C4)CC3C5)nn1-c1ccc(-c3cnc4cc(C(=O)NCCCN(C)CCCNC(=O)CN5CCN(CC(=O)OC(C)(C)C)CCN(CC(=O)OC(C)(C)C)CCN(CC(=O)OC(C)(C)C)CC5)cnc4c3)cc1C(C)C)OCO2] | 1468 [M + H]⁺ |
| 340 | tri-tert-Butyl 2,2′,2″-(10- (2-((3-((3-(5-(4-(3- (((5r,7r)-2-(tert- butoxycarbonyl)adamantan- 2-yl)carbamoyl)-5-(5- methoxybenzo[d][1,3] dioxol-4-yl)-1H-pyrazol-1- yl)-3-isopropylphenyl)-3- fluoropicolinamido)propyl) (methyl)amino)propyl) amino)-2-oxoethyl)- 1,4,7,10- tetraazacyclododecane- 1,4,7-triyl)triacetate | 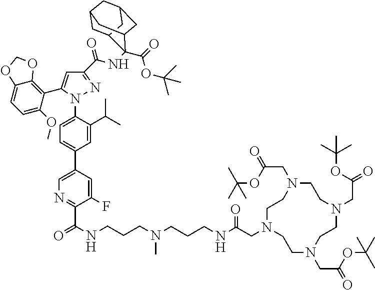 [SMILES: COc1ccc2c(c1-c1cc(C(=O)NC3(C(=O)OC(C)(C)C)C4CC5CC(C4)CC3C5)nn1-c1ccc(-c3cnc(C(=O)NCCCN(C)CCCNC(=O)CN4CCN(CC(=O)OC(C)(C)C)CCN(CC(=O)OC(C)(C)C)CCN(CC(=O)OC(C)(C)C)CC4)c(F)c3)cc1C(C)C)OCO2] | 1435 [M + H]⁺ |
| 341 | tri-tert-Butyl 2,2′,2″-(10- (2-((3-((3-(4′-(3-(((5r,7r)- 2-(tert- butoxycarbonyl)adamantan- 2-yl)carbamoyl)-5-(5- methoxybenzo[d][1,3] dioxol-4-yl)-1H-pyrazol-1- yl)-2,6-difluoro-3′- isopropyl-[1,1′- biphenyl]-4- carboxamido)propyl) (methyl)amino)propyl)amino)- 2-oxoethyl)-1,4,7,10- tetraazacyclododecane- 1,4,7-triyl)triacetate | 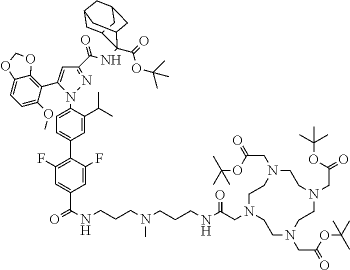 [SMILES: COc1ccc2c(c1-c1cc(C(=O)NC3(C(=O)OC(C)(C)C)C4CC5CC(C4)CC3C5)nn1-c1ccc(-c3c(F)cc(C(=O)NCCCN(C)CCCNC(=O)CN4CCN(CC(=O)OC(C)(C)C)CCN(CC(=O)OC(C)(C)C)CCN(CC(=O)OC(C)(C)C)CC4)cc3F)cc1C(C)C)OCO2] | 1452 [M + H]⁺ |
| 342 | tri-tert-Butyl 2,2′,2″-(10- (2-((3-((3-(4′-(3-(((5r,7r)- 2-(tert- butoxycarbonyl)adamantan- 2-yl)carbamoyl)-5-(5- methoxybenzo[d][1,3]dioxol- 4-yl)-1H-pyrazol-1- yl)-3′-isopropyl-3- methoxy-[1,1′-biphenyl]-4- carboxamido)propyl) (methyl)amino)propyl)amino)- 2-oxoethyl)-1,4,7,10- tetraazacyclododecane- 1,4,7-triyl)triacetate | 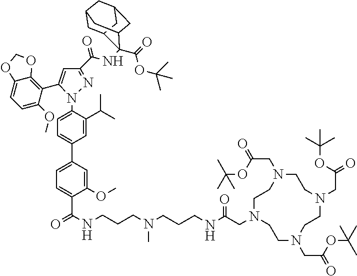 [SMILES: COc1cc(-c2ccc(-n3nc(C(=O)NC4(C(=O)OC(C)(C)C)C5CC6CC(C5)CC4C6)cc3-c3c(OC)ccc4c3OCO4)c(C(C)C)c2)ccc1C(=O)NCCCN(C)CCCNC(=O)CN1CCN(CC(=O)OC(C)(C)C)CCN(CC(=O)OC(C)(C)C)CCN(CC(=O)OC(C)(C)C)CC1] | 1446 [M + H]⁺ |

### Preparation 59

### tert-Butyl (5r,7r)-2-(1-(2-isopropyl-4-((N-methyl-N-(3-(methyl(3-(methylamino)propyl)amino)propyl)sulfamoyl)amino)phenyl)-5-(5-methoxybenzo[d][1,3]dioxol-4-yl)-1H-pyrazole-3-carboxamido)adamantane-2-carboxylate

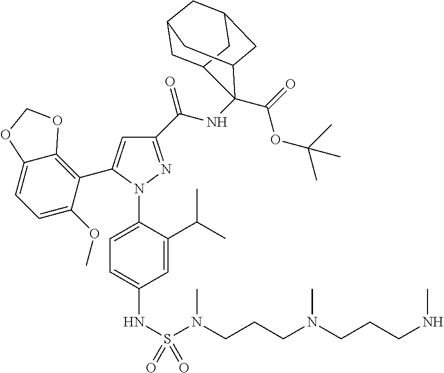 [SMILES: CNCCCN(C)CCCN(C)S(=O)(=O)Nc1ccc(-n2nc(C(=O)NC3(C(=O)OC(C)(C)C)C4CC5CC(C4)CC3C5)cc2-c2c(OC)ccc3c2OCO3)c(C(C)C)c1]

A soln of tert-butyl (5r,7r)-2-(1-(4-amino-2-isopropylphenyl)-5-(5-methoxybenzo[d][1,3]dioxol-4-yl)-1H-pyrazole-3-carboxamido)adamantane-2-carboxylate (50 mg, 80 μmol) in DMF (2 mL) at 0° C. was treated with 1-(fluorosulfonyl)-2,3-dimethyl-1H-imidazol-3-ium trifluoromethanesulfonate (52 mg, 0.16 mmol). After stirring at 0° C. for 2 h the mixture was treated with N,N-bis[3-(methylamino)propyl]methylamine (69 mg, 40 mmol) in DMF (0.5 mL). After stirring for 19 h, the mixture was purified by reversed-phase flash chromatography with the following conditions: column, C18; mobile phase, 10 to 100% ACN in H₂O (0.1% FA) to afford title compound (9 mg, 10%) as a colorless oil. MS ES+ m/z 864 [M+H]⁺.

### Preparation 60

### tert-Butyl (5r,7r)-2-(1-(2-isopropyl-4-(3-methyl-3-(3-(methyl(3-(methylamino)propyl)amino)propyl) ureido) phenyl)-5-(5-methoxybenzo[d][1,3]dioxol-4-yl)-1H-pyrazole-3-carboxamido)adamantane-2-carboxylate

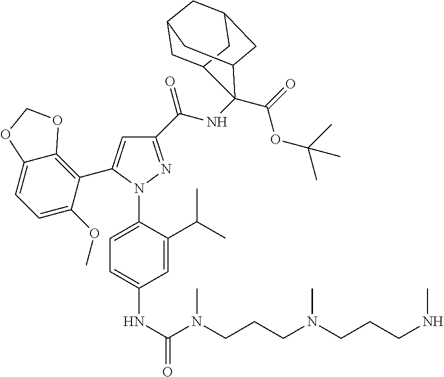 [SMILES: CNCCCN(C)CCCN(C)C(=O)Nc1ccc(-n2nc(C(=O)NC3(C(=O)OC(C)(C)C)C4CC5CC(C4)CC3C5)cc2-c2c(OC)ccc3c2OCO3)c(C(C)C)c1]

A soln of tert-butyl (5r,7r)-2-(1-(4-amino-2-isopropylphenyl)-5-(5-methoxybenzo[d][1,3]dioxol-4-yl)-1H-pyrazole-3-carboxamido)adamantane-2-carboxylate (50 mg, 80 μmol), DIEA (0.14 mL, 0.80 mmol) in DCM (4 mL) was stirred at 0° C. for 5 minutes before dropwise treatment with triphosgene (0.12 g, 0.40 mmol) in DCM (0.5 mL). After stirring at 0° C. for 5 mins, the mixture was treated with N1,N3-dimethyl-N1-(3-(methylamino)propyl)propane-1,3-diamine (0.28 g, 1.6 mmol). The mixture was slowly warmed to RT, and stirred for 3 h before treatment with ice H₂O (1 mL). After warming to RT the mixture was diluted with CHCl₃/iPrOH (3:1) (30 mL), washed with sat. aq Na₂CO₃ soln (4 mL) and brine soln (4 mL). The organic layer was dried with Na₂SO₄, concentrated and purified by silica gel column chromatography EA/Hep (0 to 100%) followed by (20% MeOH (8 N NH₃) in DCM)/Hep (0 to 100%) to afford title compound (47.2 mg, 72%). MS ES+ m/z 829 [M+H]⁺.

The following compounds were prepared in a manner essentially analogous to the preparation method of tert-Butyl (5r,7r)-2-(1-(2-isopropyl-4-(3-methyl-3-(3-(methyl(3-(methylamino)propyl)amino)propyl) ureido) phenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 13

| Prep | | |
|---|---|---|
| # | Chemical Name | Structure |
| 61 | tert-Butyl (5r,7r)-2-(1-(4- ((((3- (dimethylamino)propyl) thio)carbonyl)amino)-2- isopropylphenyl)-5-(5- methoxybenzo[d][1,3] dioxol-4-yl)-1H-pyrazole-3- carboxamido)adamantane- 2-carboxylate | 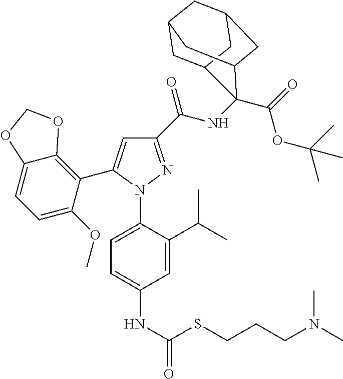 [SMILES: COc1ccc2c(c1-c1cc(C(=O)NC3(C(=O)OC(C)(C)C)C4CC5CC(C4)CC3C5)nn1-c1ccc(NC(=O)SCCCN(C)C)cc1C(C)C)OCO2] |
| 62 | tri-tert-Butyl 2,2′,2″-(10- (2-((3-((3-(((4-(3- (((5r,7r)-2-(tert- butoxycarbonyl)adamantan- 2-yl)carbamoyl)-5-(5- methoxybenzo[d][1,3] dioxol-4-yl)-1H-pyrazol-1- yl)-3- isopropylphenyl)carbamoyl) oxy)propyl)(methyl)amino) propyl)(methyl)amino)- 2-oxoethyl)-1,4,7,10- tetraazacyclododecane- 1,4,7-triyl)triacetate | 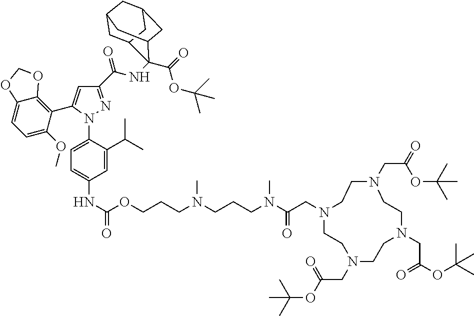 [SMILES: COc1ccc2c(c1-c1cc(C(=O)NC3(C(=O)OC(C)(C)C)C4CC5CC(C4)CC3C5)nn1-c1ccc(NC(=O)OCCCN(C)CCCN(C)C(=O)CN3CCN(CC(=O)OC(C)(C)C)CCN(CC(=O)OC(C)(C)C)CCN(CC(=O)OC(C)(C)C)CC3)cc1C(C)C)OCO2] |
| 63 | tri-tert-Butyl 2,2′,2″-(10- (2-((3-((3-(3-(4-(3- (((5r,7r)-2-(tert- butoxycarbonyl)adamantan- 2-yl)carbamoyl)-5-(5- methoxybenzo[d][1,3] dioxol-4-yl)-1H-pyrazol-1- yl)-3- isopropylphenyl)ureido) propyl)(methyl)amino) propyl)(methyl)amino)-2- oxoethyl)-1,4,7,10- tetraazacyclododecane- 1,4,7-triyl)triacetate | 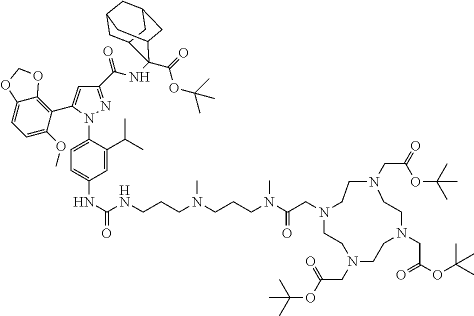 [SMILES: COc1ccc2c(c1-c1cc(C(=O)NC3(C(=O)OC(C)(C)C)C4CC5CC(C4)CC3C5)nn1-c1ccc(NC(=O)NCCCN(C)CCCN(C)C(=O)CN3CCN(CC(=O)OC(C)(C)C)CCN(CC(=O)OC(C)(C)C)CCN(CC(=O)OC(C)(C)C)CC3)cc1C(C)C)OCO2] |
| 64 | tert-Butyl (5r,7r)-2-(1- (4′-(((3- (dimethylamino)propoxy) carbonyl)(methyl)amino)- 3-isopropyl-[1,1′- biphenyl]-4-yl)-5-(5- methoxybenzo[d][1,3] dioxol-4-yl)-1H-pyrazole-3- carboxamido)adamantane- 2-carboxylate | 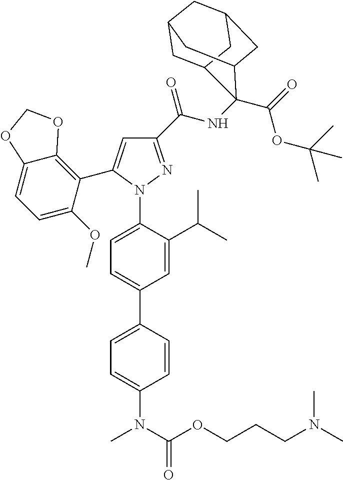 [SMILES: COc1ccc2c(c1-c1cc(C(=O)NC3(C(=O)OC(C)(C)C)C4CC5CC(C4)CC3C5)nn1-c1ccc(-c3ccc(N(C)C(=O)OCCCN(C)C)cc3)cc1C(C)C)OCO2] |
| 65 | tert-Butyl (5r,7r)-2-(1- (4′-(3-(3- (dimethylamino)propyl)- 1,3-dimethylureido)-3- isopropyl-[1,1′- biphenyl]-4-yl)-5-(5- methoxybenzo[d][1,3] dioxol-4-yl)-1H-pyrazole-3- carboxamido)adamantane- 2-carboxylate | 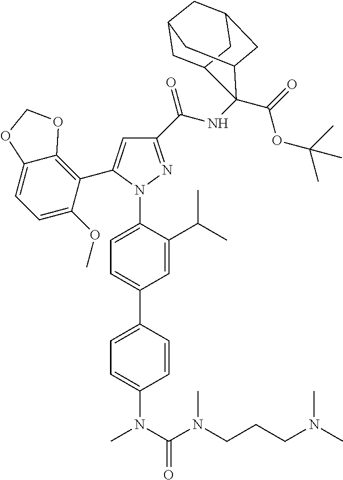 [SMILES: COc1ccc2c(c1-c1cc(C(=O)NC3(C(=O)OC(C)(C)C)C4CC5CC(C4)CC3C5)nn1-c1ccc(-c3ccc(N(C)C(=O)N(C)CCCN(C)C)cc3)cc1C(C)C)OCO2] |
| 254 | tert-Butyl (5r,7r)-2-(1-(4- (((2-((2-(1,3- dioxoisoindolin-2- yl)ethyl)(methyl)amino) ethoxy)carbonyl)amino)- 2-isopropylphenyl)-5-(5- methoxybenzo[d][1,3] dioxol-4-yl)-1H-pyrazole-3- carboxamido)adamantane- 2-carboxylate | 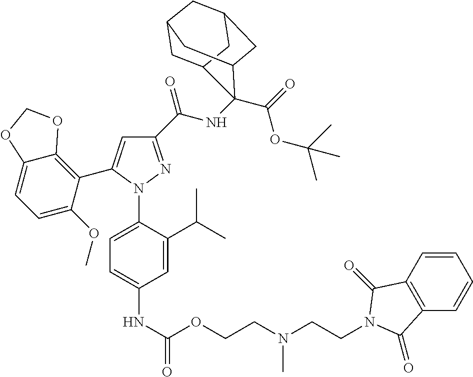 [SMILES: COc1ccc2c(c1-c1cc(C(=O)NC3(C(=O)OC(C)(C)C)C4CC5CC(C4)CC3C5)nn1-c1ccc(NC(=O)OCCN(C)CCN3C(=O)c4ccccc4C3=O)cc1C(C)C)OCO2] |
| 255 | tert-Butyl (5r,7r)-2-(1-(4- (3-(3-((3-((tert- butoxycarbonyl)amino) propyl)(methyl)amino) propyl)ureido)-2- isopropylphenyl)-5-(5- methoxybenzo[d][1,3] dioxol-4-yl)-1H-pyrazole-3- carboxamido)adamantane- 2-carboxylate | 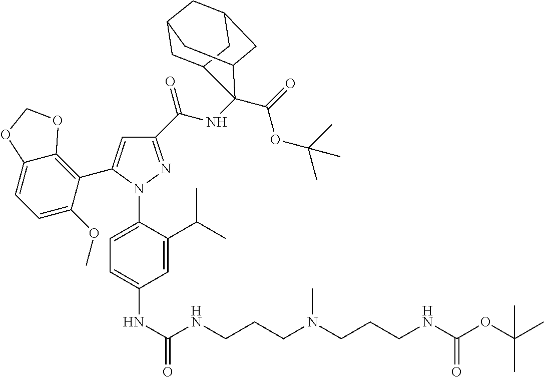 [SMILES: COc1ccc2c(c1-c1cc(C(=O)NC3(C(=O)OC(C)(C)C)C4CC5CC(C4)CC3C5)nn1-c1ccc(NC(=O)NCCCN(C)CCCNC(=O)OC(C)(C)C)cc1C(C)C)OCO2] |
| 343 | (5r,7r)-2-(1-(4-(4-((4- (tert- Butoxycarbonyl)piperazin- 1-yl)methyl)piperidine- 1-carboxamido)-2- isopropylphenyl)-5-(5- methoxybenzo[d][1,3] dioxol-4-yl)-1H-pyrazole-3- carboxamido)adamantane- 2-carboxylic acid | 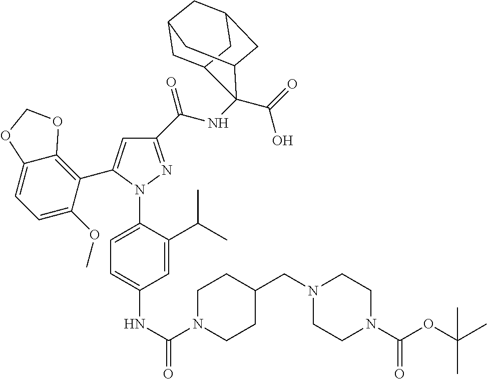 [SMILES: COc1ccc2c(c1-c1cc(C(=O)NC3(C(=O)O)C4CC5CC(C4)CC3C5)nn1-c1ccc(NC(=O)N3CCC(CN4CCN(C(=O)OC(C)(C)C)CC4)CC3)cc1C(C)C)OCO2] |
| 344 | tert-Butyl (5r,7r)-2-(1-(4- (4-(2-((tert- butoxycarbonyl)amino) ethoxy)piperidine-1- carboxamido)-2- isopropylphenyl)-5-(5- methoxybenzo[d][1,3] dioxol-4-yl)-1H-pyrazole-3- carboxamido)adamantane- 2-carboxylate | 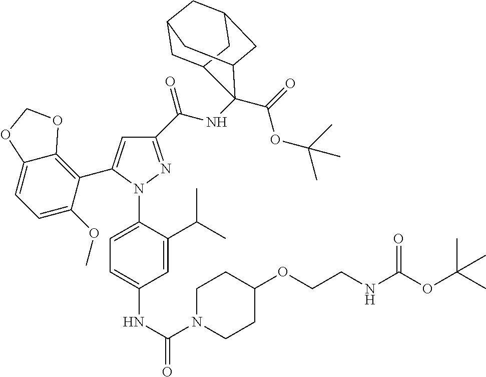 [SMILES: COc1ccc2c(c1-c1cc(C(=O)NC3(C(=O)OC(C)(C)C)C4CC5CC(C4)CC3C5)nn1-c1ccc(NC(=O)N3CCC(OCCNC(=O)OC(C)(C)C)CC3)cc1C(C)C)OCO2] |
| 345 | tert-Butyl (5r,7r)-2-(1-(4- (3-(3-((3-((tert- butoxycarbonyl)amino) propyl)(methyl)amino) propyl)ureido)-2- isopropylphenyl)-5-(5- methoxybenzo[d][1,3] dioxol-4-yl)-1H-pyrazole-3- carboxamido)adamantane- 2-carboxylate | 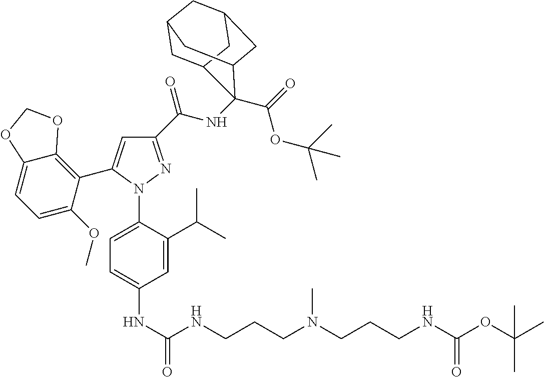 [SMILES: COc1ccc2c(c1-c1cc(C(=O)NC3(C(=O)OC(C)(C)C)C4CC5CC(C4)CC3C5)nn1-c1ccc(NC(=O)NCCCN(C)CCCNC(=O)OC(C)(C)C)cc1C(C)C)OCO2] |

| | MS |
|---|---|
| Prep | ES+ |
| # | m/z |
| 61 | 775 |
| | [M + H]⁺ |
| 62 | 1370 |
| | [M + H]⁺ |
| 63 | 1369 |
| | [M + H]⁺ |
| 64 | 849 |
| | [M + H]⁺ |
| 65 | 862 |
| | [M + H]⁺ |
| 254 | 903 |
| | [M + H]⁺ |
| 255 | 901 |
| | [M + H]⁺ |
| 343 | 939 |
| | [M + H]⁺ |
| 344 | 900 |
| | [M + H]⁺ |
| 345 | 901 |
| | [M + H]⁺ |

### Preparation 256

### tert-Butyl (5r,7r)-2-(1-(4-(3-(3-((S)-6-((((9H-fluoren-9-yl)methoxy)carbonyl)amino)-2-(4-((4-(4-iodophenyl)butanamido)methyl)benzamido)hexanamido)propyl)-3-(3-(dimethylamino)propyl) ureido)-2-isopropylphenyl)-5-(5-methoxybenzo[d][1,3]dioxol-4-yl)-1H-pyrazole-3-carboxamido)adamantane-2-carboxylate

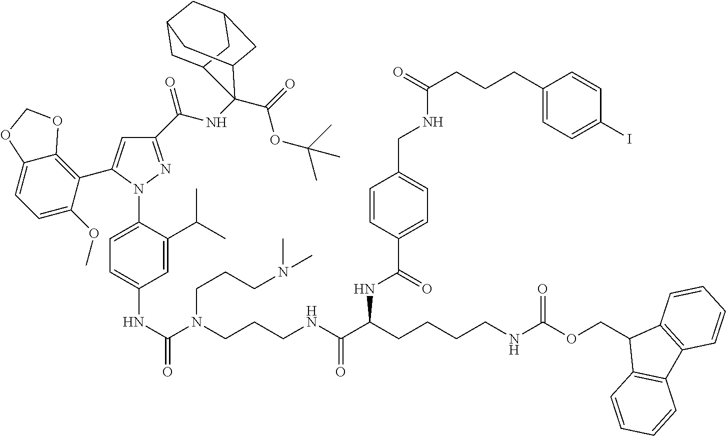 [SMILES: COc1ccc2c(c1-c1cc(C(=O)NC3(C(=O)OC(C)(C)C)C4CC5CC(C4)CC3C5)nn1-c1ccc(NC(=O)N(CCCNC(=O)[C@H](CCCCNC(=O)OCC3c4ccccc4-c4ccccc43)NC(=O)c3ccc(CNC(=O)CCCc4ccc(I)cc4)cc3)CCCN(C)C)cc1C(C)C)OCO2]

A soln of tert-butyl 2-[1-(4-amino-2-isopropylphenyl)-5-(5-methoxy-2H-1,3-benzodioxol-4-yl)pyrazole-3-amido]adamantane-2-carboxylate (250 mg, 0.398 mmol) and TEA (48 mg, 0.478 mmol) in DCM (15 mL) was treated with chloro(trichloromethoxy)methanone (94 mg, 0.478 mmol) in portions at RT under N₂. After stirring at 0° C. for 30 min, the mixture was treated with 9H-fluoren-9-ylmethyl N-[(5S)-5-[(3-{[3-(dimethylamino)propyl]amino}propyl)carbamoyl]-5-[(4-{[4-(4-iodophenyl)butanamido]methyl}phenyl)formamido]pentyl]carbamate (363 mg, 0.398 mmol) in portions at RT. After stirring for 3 h, the mixture was concentrated and purified by reversed-phase flash chromatography with the following conditions: column, C18; mobile phase, 10 to 100% ACN in H₂O (0.1% FA) to afford title compound (240 mg, 38.4%) as an off-white solid. MS ES+ m/z 1571 [M+H]⁺.

### Preparation 66

### tert-Butyl (5r,7r)-2-(1-(4-(3-bromopropoxy)-2-isopropylphenyl)-5-(5-methoxybenzo[d][1,3]dioxol-4-yl)-1H-pyrazole-3-carboxamido)adamantane-2-carboxylate

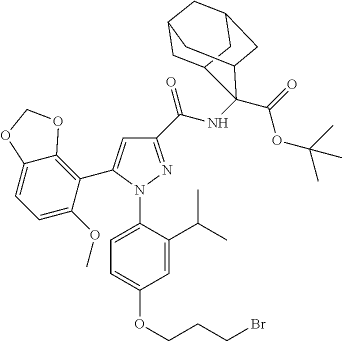 [SMILES: COc1ccc2c(c1-c1cc(C(=O)NC3(C(=O)OC(C)(C)C)C4CC5CC(C4)CC3C5)nn1-c1ccc(OCCCBr)cc1C(C)C)OCO2]

A soln of tert-butyl (5r,7r)-2-(1-(4-hydroxy-2-isopropylphenyl)-5-(5-methoxybenzo[d][1,3]dioxol-4-yl)-1H-pyrazole-3-carboxamido)adamantane-2-carboxylate (250 mg, 397 μmol), 1,3-dibromopropane (401 mg, 1.98 mmol) and K₂CO₃ (329 mg, 2.38 mmol) and DMF (8 mL) after stirring at 25° C. for 5 h the mixture was dried under vacuum and purified by silica gel column chromatography EA/Hex (0 to 50%) to afford title compound (60 mg, 20%) as white solid. MS ES+ m/z 750 [M+H]⁺.

The following compounds were prepared in a manner essentially analogous to the preparation method of tert-Butyl (5r,7r)-2-(1-(4-(3-bromopropoxy)-2-isopropylphenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 53

| Prep | | | MS ES+ |
|---|---|---|---|
| # | Chemical Name | Structure | m/z |
| 346 | tert-Butyl (5r,7r)-2-(1- (4-(2-bromoethoxy)-2- isopropylphenyl)-5-(5- methoxybenzo[d][1,3] dioxol-4-yl)-1H-pyrazole- 3-carboxamido) adamantane- 2-carboxylate | 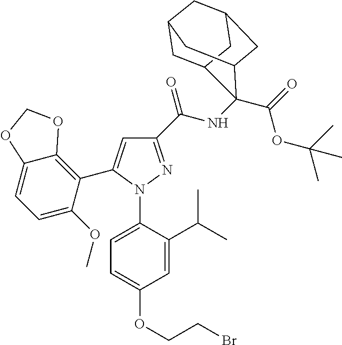 [SMILES: COc1ccc2c(c1-c1cc(C(=O)NC3(C(=O)OC(C)(C)C)C4CC5CC(C4)CC3C5)nn1-c1ccc(OCCBr)cc1C(C)C)OCO2] | 736/738 [M + H]⁺ (⁷⁹Br/⁸¹Br) |

### Preparation 398

### tert-Butyl (5R,7R)-2-(1-(4-((1R,4R)-5-(3-((tert-butoxycarbonyl)amino)propyl)-2,5-diazabicyclo[2.2.1]heptan-2-yl)-2-isopropylphenyl)-5-(5-methoxybenzo[d][1,3]dioxol-4-yl)-1H-pyrazole-3-carboxamido)adamantane-2-carboxylate

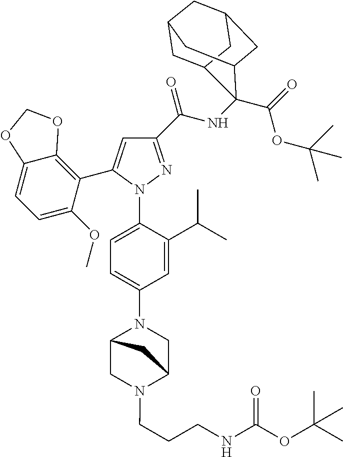 [SMILES: COc1ccc2c(c1-c1cc(C(=O)NC3(C(=O)OC(C)(C)C)C4CC5CC(C4)CC3C5)nn1-c1ccc(N3C[C@H]4C[C@@H]3CN4CCCNC(=O)OC(C)(C)C)cc1C(C)C)OCO2]

To a soln of tert-butyl(2r,5S,7S)-2-((M)-1-(4-((1R,4R)-2,5-diazabicyclo[2.2.1]heptan-2-yl)-2-isopropylphenyl)-5-(5-methoxybenzo[d][1,3]dioxol-4-yl)-1H-pyrazole-3-carboxamido)adamantane-2-carboxylate (163 mg, 0.23 mmol) and K₂CO₃ (159 mg, 1.15 mmol) in ACN (3 mL) at RT was added tert-butyl (3-bromopropyl)carbamate (137 mg, 0.57 mmol). The mixture was stirred for 18 h at 70° C. and filtered. The filtrate was purified by reversed-phase flash chromatography with the following conditions: column, C18; mobile phase, 10% to 100% ACN in H₂O (0.1% FA) to afford title compound (170 mg, 85%) as a white solid. MS ES+ m/z 868 [M+H]⁺.

The following compounds were prepared in a manner essentially analogous to the preparation method of tert-Butyl (5R,7R)-2-(1-(4-((1R,4R)-5-(3-((tert-butoxycarbonyl)amino)propyl)-2,5-diazabicyclo[2.2.1]heptan-2-yl)-2-isopropylphenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 54

| Prep | | | MS ES+ |
|---|---|---|---|
| # | Chemical Name | Structure | m/z |
| 347 | tert-Butyl (5r,7r)-2-(1- (4-(2-(3-aminopropyl)- 2,7 diazaspiro[3.5]nonan-7- yl)-2-isopropylphenyl)- 5-(5- methoxybenzo[d][1,3] dioxol-4-yl)-1H-pyrazole- 3-carboxamido) adamantane- 2-carboxylate | 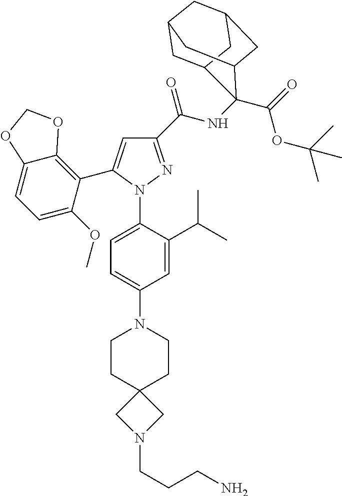 [SMILES: COc1ccc2c(c1-c1cc(C(=O)NC3(C(=O)OC(C)(C)C)C4CC5CC(C4)CC3C5)nn1-c1ccc(N3CCC4(CC3)CN(CCCN)C4)cc1C(C)C)OCO2] | 796 [M + H]⁺ |
| 348 | tert-Butyl (5r,7r)-2-(1- (4-(7-(3-((tert- butoxycarbonyl)amino) propyl)-2,7- diazaspiro[3.5]nonan-2- yl)-2-isopropylphenyl)- 5-(5- methoxybenzo[d][1,3] dioxol-4-yl)-1H-pyrazole- 3-carboxamido) adamantane- 2-carboxylate | 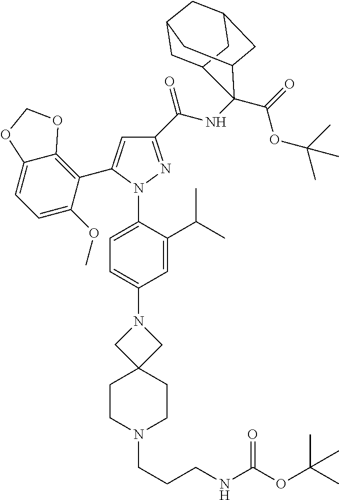 [SMILES: COc1ccc2c(c1-c1cc(C(=O)NC3(C(=O)OC(C)(C)C)C4CC5CC(C4)CC3C5)nn1-c1ccc(N3CC4(CCN(CCCNC(=O)OC(C)(C)C)CC4)C3)cc1C(C)C)OCO2] | 896 [M + H]⁺ |

### Preparation 399

### tert-Butyl (5S,7S)-2-(1-(4-((1S,4S)-5-(3-((tert-butoxycarbonyl)amino)propyl)-2,5-diazabicyclo[2.2.1]heptan-2-yl)-2-isopropylphenyl)-5-(5-methoxybenzo[d][1,3]dioxol-4-yl)-1H-pyrazole-3-carboxamido)adamantane-2-carboxylate

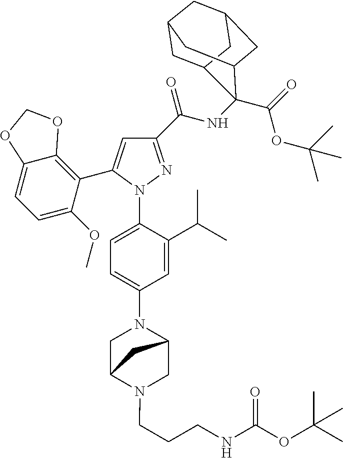 [SMILES: COc1ccc2c(c1-c1cc(C(=O)NC3(C(=O)OC(C)(C)C)C4CC5CC(C4)CC3C5)nn1-c1ccc(N3C[C@@H]4C[C@H]3CN4CCCNC(=O)OC(C)(C)C)cc1C(C)C)OCO2]

To a mixture of tert-butyl(2r,5S,7S)-2-((M)-1-(4-((1S,4S)-2,5-diazabicyclo[2.2.1]heptan-2-yl)-2-isopropylphenyl)-5-(5-methoxybenzo[d][1,3]dioxol-4-yl)-1H-pyrazole-3-carboxamido)adamantane-2-carboxylate (176 mg, 0.248 mmol) was added tert-butyl (3-oxopropyl)carbamate (64.5 mg, 0.372 mmol), STAB (158 mg, 0.744 mmol), DCM (5 mL), and AcOH (0.043 mL, 0.744 mmol) was stirred for 2 h at RT. An additional soln of tert-butyl (3-oxopropyl)carbamate (64.5 mg, 0.372 mmol), STAB (158 mg, 0.744 mmol), DCM (1 mL), and AcOH (0.04 mL, 0.744 mmol). After stirring at RT for 17 h the mixture was filtered and dried. The residue was dissolved in a mixture of MeOH/DMSO and filtered. The filtrate was purified by reversed-phase flash chromatography with the following conditions: column, C18; mobile phase, 10% to 100% ACN in H₂O (0.1% FA) to afford title compound (58 mg, 27%). MS ES+ m/z 868 [M+H]⁺.

The following compounds were prepared in a manner essentially analogous to the preparation method of tert-Butyl (5S,7S)-2-(1-(4-((1S,4S)-5-(3-((tert-butoxycarbonyl)amino)propyl)-2,5-diazabicyclo[2.2.1]heptan-2-yl)-2-isopropylphenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 55

| Prep | | | MS ES+ |
|---|---|---|---|
| # | Chemical Name | Structure | m/z |
| 349 | (5r,7r)-2-(1-(4-(9-(3- ((tert- butoxycarbonyl)amino) propyl)-3,9- diazaspiro[5.5]undecan- 3-yl)-2- isopropylphenyl)-5-(5- methoxybenzo[d][1,3] dioxol-4-yl)-1H-pyrazole- 3-carboxamido) adamantane- 2-carboxylic acid | 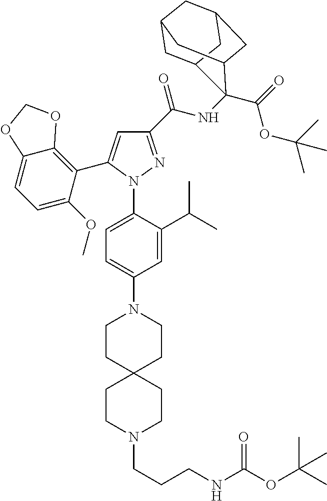 [SMILES: COc1ccc2c(c1-c1cc(C(=O)NC3(C(=O)OC(C)(C)C)C4CC5CC(C4)CC3C5)nn1-c1ccc(N3CCC4(CCN(CCCNC(=O)OC(C)(C)C)CC4)CC3)cc1C(C)C)OCO2] | 924 [M + H]⁺ |
| 350 | (5r,7r)-2-(1-(4-(9-(3- ((tert- Butoxycarbonyl)amino) propyl)-2-oxo-3,9- diazaspiro[5.5]undecan- 3-yl)-2- isopropylphenyl)-5-(5- methoxybenzo[d][1,3] dioxol-4-yl)-1H-pyrazole- 3-carboxamido) adamantane- 2-carboxylic acid | 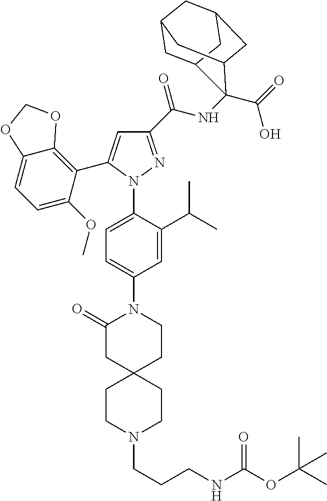 [SMILES: COc1ccc2c(c1-c1cc(C(=O)NC3(C(=O)O)C4CC5CC(C4)CC3C5)nn1-c1ccc(N3CCC4(CCN(CCCNC(=O)OC(C)(C)C)CC4)CC3=O)cc1C(C)C)OCO2] | 882 [M + H]⁺ |

### Preparation 67

### tri-tert-Butyl 2,2′,2″-(10-(2-((3-((4-((4-(3-(((5r,7r)-2-(tert-butoxycarbonyl)adamantan-2-yl)carbamoyl)-5-(5-methoxybenzo[d][1,3]dioxol-4-yl)-1H-pyrazol-1-yl)-3-isopropylphenyl)amino)-4-oxobutyl)(methyl)amino)propyl)(methyl)amino)-2-oxoethyl)-1,4,7,10-tetraazacyclododecane-1,4,7-triyl)triacetate

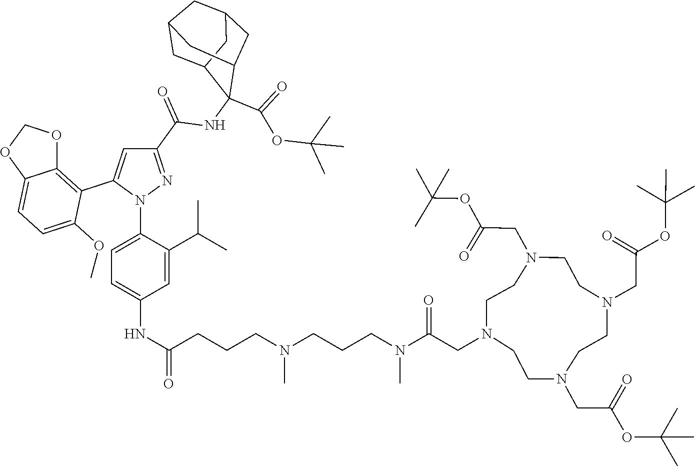 [SMILES: COc1ccc2c(c1-c1cc(C(=O)NC3(C(=O)OC(C)(C)C)C4CC5CC(C4)CC3C5)nn1-c1ccc(NC(=O)CCCN(C)CCCN(C)C(=O)CN3CCN(CC(=O)OC(C)(C)C)CCN(CC(=O)OC(C)(C)C)CCN(CC(=O)OC(C)(C)C)CC3)cc1C(C)C)OCO2]

A soln of tert-butyl (5r,7r)-2-(1-(4-amino-2-isopropylphenyl)-5-(5-methoxybenzo[d][1,3]dioxol-4-yl)-1H-pyrazole-3-carboxamido)adamantane-2-carboxylate (45 mg, 0.072 mmol), DIEA (0.05 mL, 0.29 mmol) DMAP (0.44 mg, 0.0036 mmol), 4-(methyl(3-(N-methyl-2-(4,7,10-tris(2-(tert-butoxy)-2-oxoethyl)-1,4,7,10-tetraazacyclododecan-1-yl)acetamido)propyl)amino)butanoic acid (100 mg, 0.135 mmol) and T3P (0.18 mL, 50% wt, 0.29 mmol) in degassed DMF (5 mL) was stirred under N₂ at 22° C. for 1 hour. After heating at 60° C. for 1 h the mixture was charged with additional DMAP (0.44 mg, 0.0036 mmol) and T3P (0.09 mL, 50% Wt, 0.14 mmol) and purged with N₂ and stirred for 1 h at 60° C. The mixture was charged with additional T3P (0.39 mL, 50% Wt, 0.64 mmol) and DIEA (0.07 mL, 0.43 mmol). After stirring at 60° C. for 2 h the mixture was concentrated and purified by reversed-phase flash chromatography with the following conditions: column, C18; mobile phase, 0% to 50% ACN in H₂O (0.1% FA) to afford title compound (33 mg, 34%). MS ES+ m/z 1354 [M+H]⁺.

The following compounds were prepared in a manner essentially analogous to the preparation method of tri-tert-Butyl 2,2′,2″-(10-(2-((3-((4-((4-(3-(((5r,7r)-2-(tert-butoxycarbonyl)adamantan-2-yl)carbamoyl)-5-(5-methoxybenzo[d][1,3]dioxol-4-yl)-1H-pyrazol-1-yl)-3-isopropylphenyl)amino)-4-oxobutyl)(methyl)amino)propyl)(methyl)amino)-2-oxoethyl)-1,4,7,10-tetraazacyclododecane-1,4,7-triyl)triacetate using the appropriate reagents, adjusting temperature, adjusting reaction time to determine completion of the reaction, and adjusting the purification system as appropriate.

TABLE 14

| | | | MS |
|---|---|---|---|
| Prep | | | ES+ |
| # | Chemical Name | Structure | m/z |
| 68 | tri-tert-Butyl 2,2′,2″-(10- (2-((3-((4-((4′-(3- (((5r,7r)-2-(tert- butoxycarbonyl) adamantan- 2-yl)carbamoyl)-5- (5- methoxybenzo[d][1,3] dioxol-4-yl)-1H-pyrazol- 1-yl)-3′-isopropyl-[1,1′- biphenyl]-4-yl)amino)- 4- oxobutyl)(methyl)amino) propyl)(methyl)amino)- 2-oxoethyl)-1,4,7,10- tetraazacyclododecane- 1,4,7-triyl)triacetate | 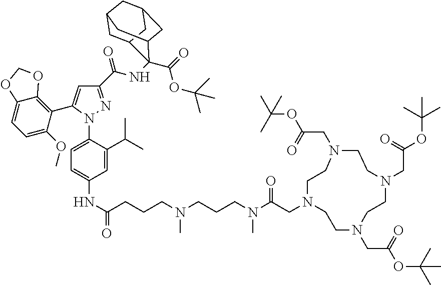 | 1430 [M + H]⁺ |
| 257 | tri-tert-Butyl 2,2′,2″-(10- (2-((3-((4-((4-(3- (((5r,7r)-2-(tert- butoxycarbonyl) adamantan- 2-yl)carbamoyl)-5- (5- methoxybenzo[d][1,3] dioxol-4-yl)-1H-pyrazol- 1-yl)-3- isopropylphenyl) amino)-4-oxobutyl) (methyl)amino) propyl)amino)-2- oxoethyl)-1,4,7,10- tetraazacyclododecane- 1,4,7-triyl)triacetate | 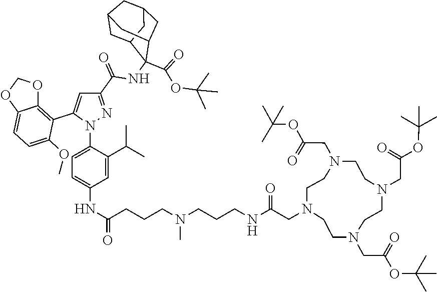 [SMILES: COc1ccc2c(c1-c1cc(C(=O)NC3(C(=O)OC(C)(C)C)C4CC5CC(C4)CC3C5)nn1-c1ccc(NC(=O)CCCN(C)CCCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 1340 [M + H]⁺ |
| 258 | (5r,7r)-2-(1-(4-(2,2- Dimethyl-4-oxo- 3,8,11,14,17-pentaoxa- 5-azanonadecan-19- amido)-2- isopropylphenyl)-5-(5- methoxybenzo[d][1,3] dioxol-4-yl)-1H-pyrazole- 3-carboxamido) adamantane- 2-carboxylic acid | 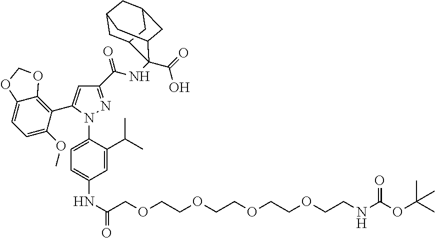 [SMILES: COc1ccc2c(c1-c1cc(C(=O)NC3(C(=O)O)C4CC5CC(C4)CC3C5)nn1-c1ccc(NC(=O)COCCOCCOCCOCCNC(=O)OC(C)(C)C)cc1C(C)C)OCO2] | 863 [M − tBu]⁺ |
| 259 | tri-tert-Butyl 2,2′,2″-(10- (2-((3-((4-((4-(3- (((5r,7r)-2-(tert- butoxycarbonyl) adamantan- 2-yl)carbamoyl)-5- (5- methoxybenzo[d][1,3] dioxol-4-yl)-1H-pyrazol- 1-yl)-3- isopropylphenyl)amino)- 4-oxobutyl)(3- (dimethylamino)propyl) amino)propyl)(methyl) amino)-2-oxoethyl)- 1,4,7,10- tetraazacyclododecane- 1,4,7-triyl)triacetate | 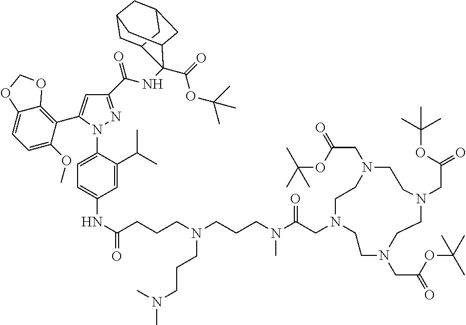 [SMILES: COc1ccc2c(c1-c1cc(C(=O)NC3(C(=O)OC(C)(C)C)C4CC5CC(C4)CC3C5)nn1-c1ccc(NC(=O)CCCN(CCCN(C)C)CCCN(C)C(=O)CN3CCN(CC(=O)OC(C)(C)C)CCN(CC(=O)OC(C)(C)C)CCN(CC(=O)OC(C)(C)C)CC3)cc1C(C)C)OCO2] | 1425 [M + H]⁺ |
| 260 | tri-tert-Butyl 2,2′,2″-(10- (2-((3-((4-((4-(3- (((5r,7r)-2-(tert- butoxycarbonyl) adamantan- 2-yl)carbamoyl)-5- (5- methoxybenzo[d][1,3] dioxol-4-yl)-1H-pyrazol- 1-yl)-3- isopropylphenyl)amino)- 4-oxobutyl)((1- methylpyrrolidin-3- yl)methyl)amino)propyl) (methyl)amino)-2- oxoethyl)-1,4,7,10- tetraazacyclododecane- 1,4,7-triyl)triacetate | 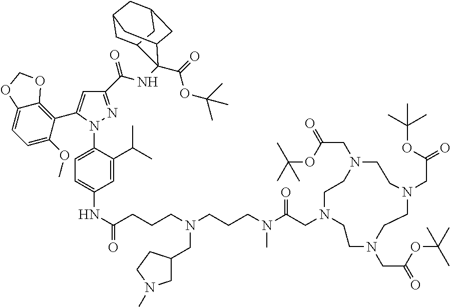 [SMILES: COc1ccc2c(c1-c1cc(C(=O)NC3(C(=O)OC(C)(C)C)C4CC5CC(C4)CC3C5)nn1-c1ccc(NC(=O)CCCN(CCCN(C)C(=O)CN3CCN(CC(=O)OC(C)(C)C)CCN(CC(=O)OC(C)(C)C)CCN(CC(=O)OC(C)(C)C)CC3)CC3CCN(C)C3)cc1C(C)C)OCO2] | 1437 [M + H]⁺ |
| 351 | tert-Butyl (5r,7r)-2-(1- (4-(2-(2-(2- bromoethoxy)ethoxy) acetamido)-2- isopropylphenyl)-5-(5- methoxybenzo[d][1,3] dioxo1-4-yl)-1H- pyrazole-3- carboxamido) adamantane- 2-carboxylate | 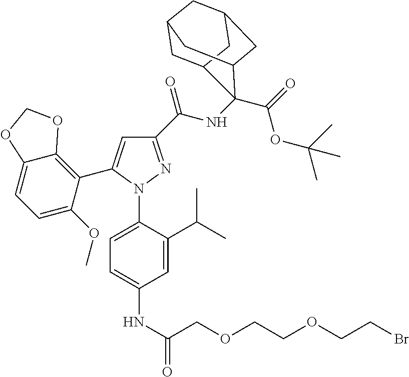 | 1437 [M + H]⁺ |

### Preparation 69

### tri-tert-Butyl 2,2′,2″-(10-(2-((3-((3-(4-(3-(((5r,7r)-2-(tert-butoxycarbonyl)adamantan-2-yl)carbamoyl)-5-(5-methoxybenzo[d][1,3]dioxol-4-yl)-1H-pyrazol-1-yl)-3-isopropylphenoxy)propyl)(methyl)amino)propyl)(methyl)amino)-2-oxoethyl)-1,4,7,10-tetraazacyclododecane-1,4,7-triyl)triacetate

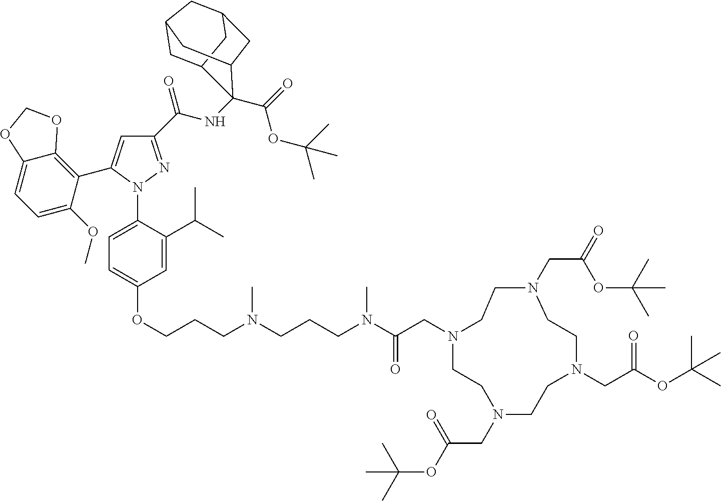 [SMILES: COc1ccc2c(c1-c1cc(C(=O)NC3(C(=O)OC(C)(C)C)C4CC5CC(C4)CC3C5)nn1-c1ccc(OCCCN(C)CCCN(C)C(=O)CN3CCN(CC(=O)OC(C)(C)C)CCN(CC(=O)OC(C)(C)C)CCN(CC(=O)OC(C)(C)C)CC3)cc1C(C)C)OCO2]

A soln of tert-butyl (5r,7r)-2-(1-(4-(3-bromopropoxy)-2-isopropylphenyl)-5-(5-methoxybenzo[d][1,3]dioxol-4-yl)-1H-pyrazole-3-carboxamido)adamantane-2-carboxylate (60 mg, 0.08 mmol), tri-tert-butyl 2,2′,2″-(10-(2-(methyl(3-(methylamino)propyl)amino)-2-oxoethyl)-1,4,7,10-tetraazacyclododecane-1,4,7-triyl)triacetate (53 mg, 0.08 mmol) and K₂CO₃ (44 mg, 0.32 mmol) NaI (24 mg, 0.16 mmol) in MeCN (8 mL) stirred for 3 h at 80° C. The reaction was dried under vacuum and purified by reversed-phase flash chromatography with the following conditions: column, C18; mobile phase, 0% to 30% ACN in H₂O (0.1% FA) to afford title compound (20 mg, 19%) as white solid. MS ES+ m/z 1327 [M+H]⁺.

The following compounds were prepared in a manner essentially analogous to the preparation method of tri-tert-Butyl 2,2′,2″-(10-(2-((3-((3-(4-(3-(((5r,7r)-2-(tert-butoxycarbonyl)adamantan-2-yl)carbamoyl)-5-(5-methoxybenzo[d][1,3]dioxol-4-yl)-1H-pyrazol-1-yl)-3-isopropylphenoxy)propyl)(methyl)amino)propyl)(methyl)amino)-2-oxoethyl)-1,4,7,10-tetraazacyclododecane-1,4,7-triyl)triacetate using the appropriate reagents, adjusting temperature, adjusting reaction time to determine completion of the reaction, and adjusting the purification system as appropriate.

TABLE 15

| | | | MS |
|---|---|---|---|
| Prep | | | ES+ |
| # | Chemical Name | Structure | m/z |
| 70 | tert-Butyl (5r,7r)-2-(1-(4- (3-((2-((tert- butoxycarbonyl)amino)ethyl) (methyl)amino)propoxy)- 2-isopropylphenyl)- 5-(5- methoxybenzo[d][1,3] dioxol-4-yl)-1H-pyrazole-3- carboxamido)adamantane- 2-carboxylate | 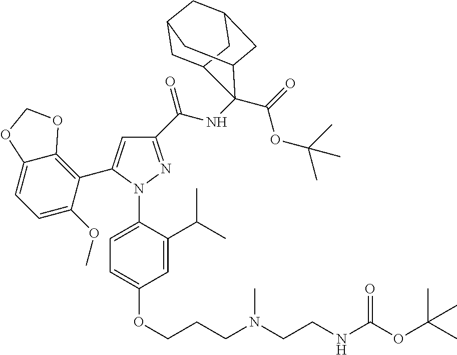 [SMILES: COc1ccc2c(c1-c1cc(C(=O)NC3(C(=O)OC(C)(C)C)C4CC5CC(C4)CC3C5)nn1-c1ccc(OCCCN(C)CCNC(=O)OC(C)(C)C)cc1C(C)C)OCO2] | 845 [M + H]⁺ |
| 261 | tert-butyl (5r,7r)-2-(1-(4- (3-((3-((tert- butoxycarbonyl)amino) propyl)(methyl)amino) propoxy)-2- isopropylphenyl)-5-(5- methoxybenzo[d][1,3] dioxol-4-yl)-1H-pyrazole-3- carboxamido)adamantane- 2-carboxylate | 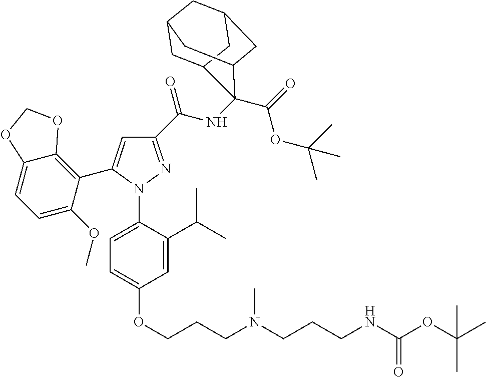 [SMILES: COc1ccc2c(c1-c1cc(C(=O)NC3(C(=O)OC(C)(C)C)C4CC5CC(C4)CC3C5)nn1-c1ccc(OCCCN(C)CCCNC(=O)OC(C)(C)C)cc1C(C)C)OCO2] | 858 [M + H]⁺ |
| 352 | tri-tert-Butyl 2,2′,2″-(10- (1-((4-(3-(((5r,7r)-2-(tert- butoxycarbonyl)adamantan- 2-yl)carbamoyl)-5-(5- methoxybenzo[d][1,3] dioxol-4-yl)-1H-pyrazol-1- yl)-3- isopropylphenyl)amino)- 13-methyl-1,18-dioxo- 3,6-dioxa-9,13,17- triazanonadecan-19-yl)- 1,4,7,10- tetraazacyclododecane- 1,4,7-triyl)triacetate | 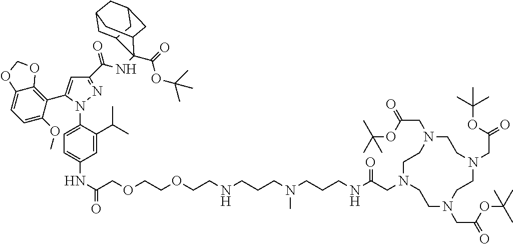 [SMILES: COc1ccc2c(c1-c1cc(C(=O)NC3(C(=O)OC(C)(C)C)C4CC5CC(C4)CC3C5)nn1-c1ccc(NC(=O)COCCOCCNCCCN(C)CCCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 1457 [M + H]⁺ |
| 353 | tert-Butyl (5r,7r)-2-(1-(4- (5-(3-((3-((tert- butoxycarbonyl)amino) propyl)(methyl) amino)propyl)- 1,3,4-oxadiazol-2-yl)- 2-isopropylphenyl)-5-(5- methoxybenzo[d][1,3] dioxol-4-yl)-1H-pyrazole- 3-carboxamido) adamantane-2-carboxylate | 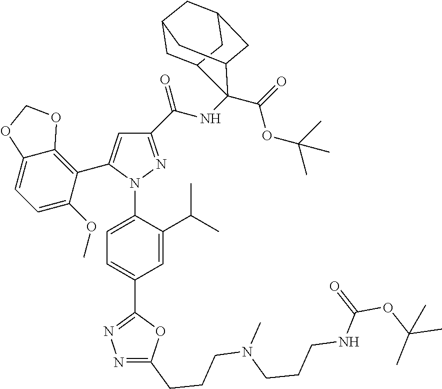 [SMILES: COc1ccc2c(c1-c1cc(C(=O)NC3(C(=O)OC(C)(C)C)C4CC5CC(C4)CC3C5)nn1-c1ccc(-c3nnc(CCCN(C)CCCNC(=O)OC(C)(C)C)o3)cc1C(C)C)OCO2] | 911 [M + H]⁺ |
| 354 | tert-Butyl (5r,7r)-2-(1-(4- (2-(4-(3-((tert- butoxycarbonyl)amino) propyl)piperazin-1- yl)ethoxy)-2- isopropylphenyl)-5-(5- methoxybenzo[d][1,3] dioxol-4-yl)-1H-pyrazole-3- carboxamido)adamantane- 2-carboxylate | 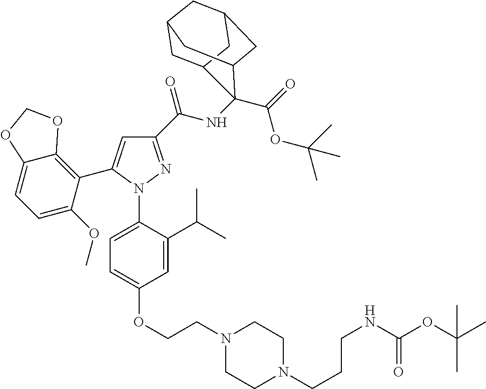 [SMILES: COc1ccc2c(c1-c1cc(C(=O)NC3(C(=O)OC(C)(C)C)C4CC5CC(C4)CC3C5)nn1-c1ccc(OCCN3CCN(CCCNC(=O)OC(C)(C)C)CC3)cc1C(C)C)OCO2] | 900 [M + H]⁺ |

### Preparation 262

### tert-Butyl (5r,7r)-2-(1-(2-isopropyl-4-(methyl(2,2,14,18-tetramethyl-4,13-dioxo-3,8,11-trioxa-5,14,18-triazahenicosan-21-yl)amino)phenyl)-5-(5-methoxybenzo[d][1,3]dioxol-4-yl)-1H-pyrazole-3-carboxamido)adamantane-2-carboxylate

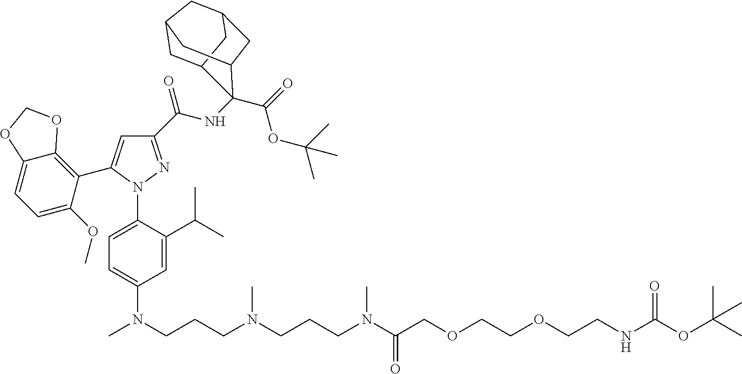 [SMILES: COc1ccc2c(c1-c1cc(C(=O)NC3(C(=O)OC(C)(C)C)C4CC5CC(C4)CC3C5)nn1-c1ccc(N(C)CCCN(C)CCCN(C)C(=O)COCCOCCNC(=O)OC(C)(C)C)cc1C(C)C)OCO2]

A soln of (2-{2-[(tert-butoxycarbonyl)amino]ethoxy}ethoxy)acetic acid (140 mg, 0.535 mmol) and HATU (271 mg, 0.714 mmol) in DMF (4 mL) at RT under N₂ was treated with DIEA (138 mg, 1.071 mmol). After stirring for 30 min, the mixture was treated with tert-butyl 2-(1-{2-isopropyl-4-[methyl (3-{methyl[3-(methylamino)propyl]amino}propyl)amino]phenyl}-5-(5-methoxy-2H-1,3-benzodioxol-4-yl)pyrazole-3-amido)adamantane-2-carboxylate (280 mg, 0.357 mmol). After stirring at RT for 2 h, the mixture was concentrated and purified by reversed-phase flash chromatography with the following conditions: column, C18; mobile phase, 10% to 100% ACN in H₂O (0.1% FA) to provide title compound (130 mg, 35%) as a yellow solid. MS ES+ m/z 1031 [M+H]⁺.

### Preparation 263

### tert-Butyl (5r,7r)-2-(1-(4-((3-((3-(3-bromopropoxy)propyl)(methyl)amino)propyl)(methyl)amino)-2-isopropylphenyl)-5-(5-methoxybenzo[d][1,3]dioxol-4-yl)-1H-pyrazole-3-carboxamido)adamantane-2-carboxylate

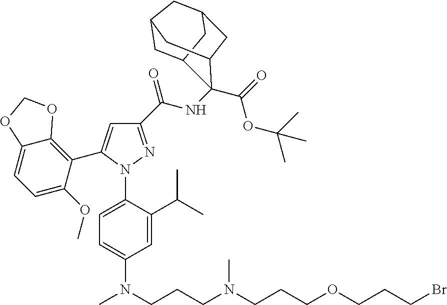 [SMILES: COc1ccc2c(c1-c1cc(C(=O)NC3(C(=O)OC(C)(C)C)C4CC5CC(C4)CC3C5)nn1-c1ccc(N(C)CCCN(C)CCCOCCCBr)cc1C(C)C)OCO2]

A soln of tert-butyl (5r,7r)-2-(1-(2-isopropyl-4-(methyl(3-(methylamino)propyl)amino)phenyl)-5-(5-methoxybenzo[d][1,3]dioxol-4-yl)-1H-pyrazole-3-carboxamido)adamantane-2-carboxylate (20 mg, 0.028 mmol) in DCM (1 mL) was treated with Propane, 1,1′-oxybis[3-bromo-(15 mg, 0.056 mmol) and K₂CO₃ (23 mg, 0.17 mmol). After stirring at RT for 18 the mixture was concentrated and purified by silica gel column chromatography (20% MeOH (8 N NH₃) in DCM)/hep (0% to 100%) to afford title compound (12.2 mg, 49%).

### Preparation 264

### tri-tert-Butyl 2,2′,2″-(10-(3-(3-((3-((4-(3-(((5r,7r)-2-(tert-butoxycarbonyl)adamantan-2-yl)carbamoyl)-5-(5-methoxybenzo[d][1,3]dioxol-4-yl)-1H-pyrazol-1-yl)-3-isopropylphenyl)(methyl)amino)propyl)(methyl)amino)propoxy)propyl)-1,4,7,10-tetraazacyclododecane-1,4,7-triyl)triacetate

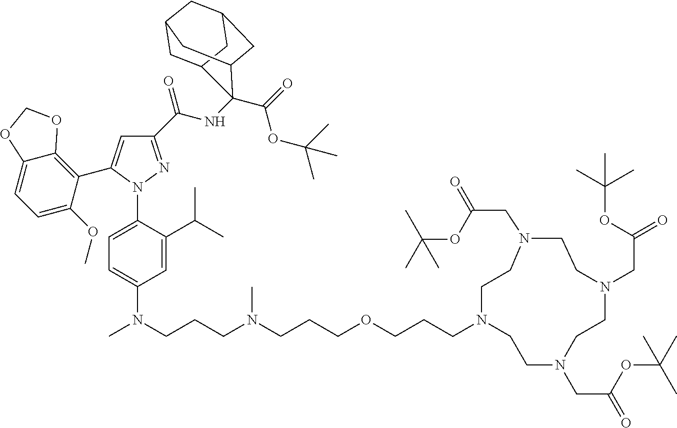 [SMILES: COc1ccc2c(c1-c1cc(C(=O)NC3(C(=O)OC(C)(C)C)C4CC5CC(C4)CC3C5)nn1-c1ccc(N(C)CCCN(C)CCCOCCCN3CCN(CC(=O)OC(C)(C)C)CCN(CC(=O)OC(C)(C)C)CCN(CC(=O)OC(C)(C)C)CC3)cc1C(C)C)OCO2]

A soln of tert-butyl (5r,7r)-2-(1-(4-((3-((3-(3-bromopropoxy)propyl)(methyl)amino)propyl)(methyl)amino)-2-isopropylphenyl)-5-(5-methoxybenzo[d][1,3]dioxol-4-yl)-1H-pyrazole-3-carboxamido)adamantane-2-carboxylate (12.2 mg, 0.014 mmol) in DMF (1 mL) was treated with tert-butyl 2-[4,7-bis(2-tert-butoxy-2-oxo-ethyl)-1,4,7,10-tetrazacyclododec-1-yl]acetate (14.1 mg, 0.027 mmol) and K₂CO₃ (11.3 mg, 0.082 mmol). After stirring at RT for 18 h, the mixture was heated to 40° C. for 4 h. The mixture was concentrated and purified by silica gel column chromatography EA/hep (0% to 100%) to afford title compound (16 mg, 44%). MS ES+ m/z 1327 [M+H]⁺.

### Preparation 71

### (5r,7r)-2-(1-(4-(3-((2-Aminoethyl)(methyl)amino)propoxy)-2-isopropylphenyl)-5-(5-methoxybenzo[d][1,3]dioxol-4-yl)-1H-pyrazole-3-carboxamido)adamantane-2-carboxylic acid

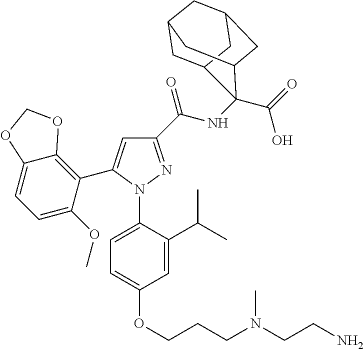 [SMILES: COc1ccc2c(c1-c1cc(C(=O)NC3(C(=O)O)C4CC5CC(C4)CC3C5)nn1-c1ccc(OCCCN(C)CCN)cc1C(C)C)OCO2]

A soln of tert-butyl (5r,7r)-2-(1-(4-(3-((2-((tert-butoxycarbonyl)amino)ethyl)(methyl)amino)propoxy)-2-isopropylphenyl)-5-(5-methoxybenzo[d][1,3]dioxol-4-yl)-1H-pyrazole-3-carboxamido)adamantane-2-carboxylate (160 mg, 0.19 mmol) in DCM (8 mL) was treated with TFA (8 mL). After stirring at 40° C. for 3 h the mixture was concentrated under vacuum to give crude title compound (130 mg, 77%) as a colorless foam. MS ES+ m/z 688 [M+H]⁺.

The following compounds were prepared in a manner essentially analogous to the preparation method of (5r,7r)-2-(1-(4-(3-((2-Aminoethyl)(methyl)amino)propoxy)-2-isopropylphenyl)-5-(5-methoxybenzo[d][1,3]dioxol-4-yl)-1H-pyrazole-3-carboxamido)adamantane-2-carboxylic acid using the appropriate reagents, adjusting temperature, adjusting reaction time to determine completion of the reaction, and adjusting the purification system as appropriate.

TABLE 16

| | | | MS |
|---|---|---|---|
| Prep | | | ES+ |
| # | Chemical Name | Structure | m/z |
| 72 | (5r,7r)-2-(1-(2- Isopropyl-4-(1-(3- (methyl(3- (methylamino)propyl) amino)propyl)-1H- 1,2,3-triazol-4- yl)phenyl)-5-(5- methoxybenzo[d][1,3] dioxol-4-yl)-1H- pyrazole-3-carboxamido) adamantane- 2-carboxylic acid | 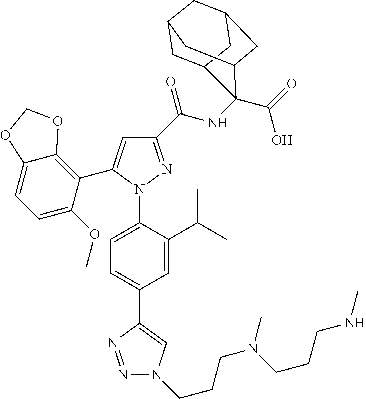 [SMILES: CNCCCN(C)CCCn1cc(-c2ccc(-n3nc(C(=O)NC4(C(=O)O)C5CC6CC(C5)CC4C6)cc3-c3c(OC)ccc4c3OCO4)c(C(C)C)c2)nn1] | 768 [M + H]⁺ |
| 265 | (5r,7r)-2-(1-(4-(3-((3- Aminopropyl)(methyl) amino)propoxy)-2- isopropylphenyl)-5-(5- methoxybenzo[d][1,3] dioxol-4-yl)-1H- pyrazole-3- carboxamido) adamantane- 2-carboxylic acid | 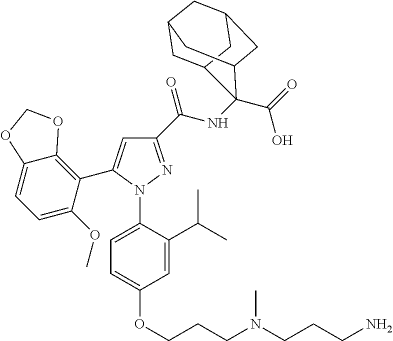 [SMILES: COc1ccc2c(c1-c1cc(C(=O)NC3(C(=O)O)C4CC5CC(C4)CC3C5)nn1-c1ccc(OCCCN(C)CCCN)cc1C(C)C)OCO2] | 702 [M + H]⁺ |
| 266 | (5r,7r)-2-(1-(4-(14- Amino-3,6,9,12- tetraoxatetradecanamido)- 2-isopropylphenyl)-5-(5- methoxybenzo[d][1,3] dioxol-4-yl)-1H- pyrazole-3- carboxamido) adamantane- 2-carboxylic acid | 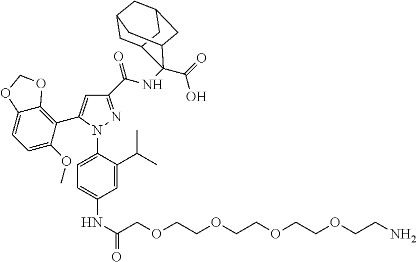 [SMILES: COc1ccc2c(c1-c1cc(C(=O)NC3(C(=O)O)C4CC5CC(C4)CC3C5)nn1-c1ccc(NC(=O)COCCOCCOCCOCCN)cc1C(C)C)OCO2] | 806 [M + H]⁺ |
| 267 | tert-Butyl (5r,7r)-2-(1- (4-(3-(3-((3- aminopropyl)(methyl) amino)propyl)ureido)- 2-isopropylphenyl)-5- (5- methoxybenzo[d][1,3] dioxol-4-yl)-1H- pyrazole-3- carboxamido) adamantane- 2-carboxylate | 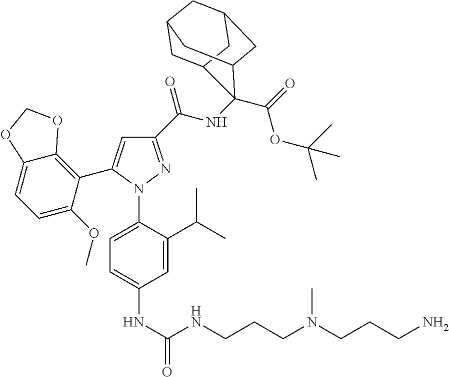 [SMILES: COc1ccc2c(c1-c1cc(C(=O)NC3(C(=O)OC(C)(C)C)C4CC5CC(C4)CC3C5)nn1-c1ccc(NC(=O)NCCCN(C)CCCN)cc1C(C)C)OCO2] | 745 [M + H]⁺ |
| 268 | (5r,7r)-2-(1-(4-(1-(3- ((3- Aminopropyl)(methyl) amino)propyl)-1H- 1,2,3-triazol-4-yl)-2- isopropylphenyl)-5-(5- methoxybenzo[d][1,3] dioxol-4-yl)-1H- pyrazole-3- carboxamido) adamantane- 2-carboxylic acid | 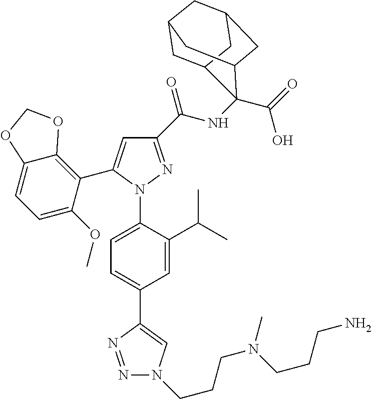 [SMILES: COc1ccc2c(c1-c1cc(C(=O)NC3(C(=O)O)C4CC5CC(C4)CC3C5)nn1-c1ccc(-c3cn(CCCN(C)CCCN)nn3)cc1C(C)C)OCO2] | 751 [M + H]⁺ |
| 269 | (5r,7r)-2-(1-(4-((1- Amino-9,13-dimethyl- 8-oxo-3,6-dioxa-9,13- diazahexadecan-16- yl)(methyl)amino)-2- isopropylphenyl)-5-(5- methoxybenzo[d][1,3] dioxol-4-yl)-1H- pyrazole-3- carboxamido) adamantane-2- carboxylic acid | 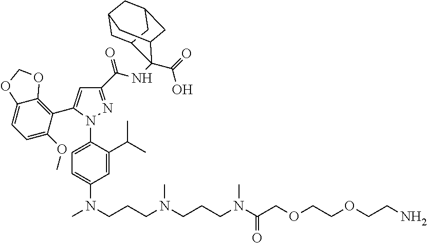 [SMILES: COc1ccc2c(c1-c1cc(C(=O)NC3(C(=O)O)C4CC5CC(C4)CC3C5)nn1-c1ccc(N(C)CCCN(C)CCCN(C)C(=O)COCCOCCN)cc1C(C)C)OCO2] | 438 [M/2 + H]⁺ |
| 355 | (5r,7r)-2-(1-(4-(9-(3- Aminopropyl)-3,9- diazaspiro[5.5] undecan-3-yl)-2- isopropylphenyl)-5-(5- methoxybenzo[d][1,3] dioxol-4-yl)-1H- pyrazole-3- carboxamido) adamantane- 2-carboxylic acid | 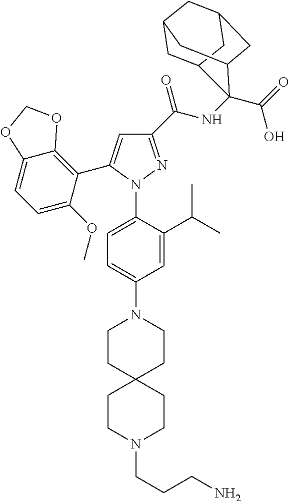 [SMILES: COc1ccc2c(c1-c1cc(C(=O)NC3(C(=O)O)C4CC5CC(C4)CC3C5)nn1-c1ccc(N3CCC4(CCN(CCCN)CC4)CC3)cc1C(C)C)OCO2] | 765 [M − H]]⁻ |
| 356 | (5r,7r)-2-(1-(2- Isopropyl-4-(4- (piperazin-1- ylmethyl)piperidine-1- carboxamido)phenyl)- 5-(5- methoxybenzo[d][1,3] dioxol-4-yl)-1H- pyrazole-3- carboxamido) adamantane-2- carboxylic acid | 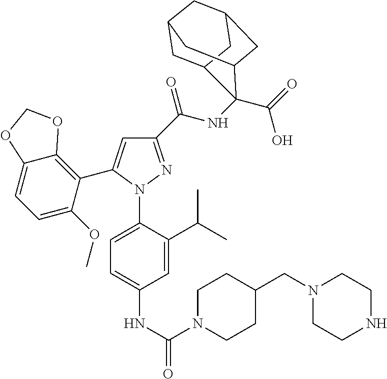 [SMILES: COc1ccc2c(c1-c1cc(C(=O)NC3(C(=O)O)C4CC5CC(C4)CC3C5)nn1-c1ccc(NC(=O)N3CCC(CN4CCNCC4)CC3)cc1C(C)C)OCO2] | 780 [M − H]]⁻ |
| 357 | (5r,7r)-2-(1-(2- isopropyl-4-(3- (piperazin-1- yl)propoxy)phenyl)-5- (5- methoxybenzo[d][1,3] dioxol-4-yl)-1H- pyrazole-3- carboxamido) adamantane- 2-carboxylic acid | 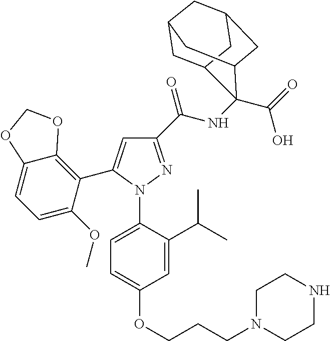 [SMILES: COc1ccc2c(c1-c1cc(C(=O)NC3(C(=O)O)C4CC5CC(C4)CC3C5)nn1-c1ccc(OCCCN3CCNCC3)cc1C(C)C)OCO2] | 700 [M + H]⁺ |
| 358 | (5r,7r)-2-(1-(2- Isopropyl-4-(2- (piperazin-1- yl)ethoxy)phenyl)-5- (5- methoxybenzo[d][1,3] dioxol-4-yl)-1H- pyrazole-3- carboxamido)adamantane- 2-carboxylic acid | 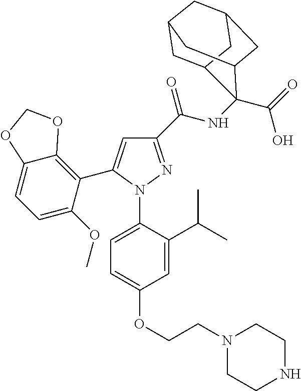 [SMILES: COc1ccc2c(c1-c1cc(C(=O)NC3(C(=O)O)C4CC5CC(C4)CC3C5)nn1-c1ccc(OCCN3CCNCC3)cc1C(C)C)OCO2] | 686 [M + H]⁺ |
| 359 | (5r,7r)-2-(1-(4-(3-(3- Aminopropoxy) propoxy)-2- isopropylphenyl)-5-(5- methoxybenzo[d][1,3] dioxol-4-yl)-1H- pyrazole-3- carboxamido)adamantane- 2-carboxylic acid | 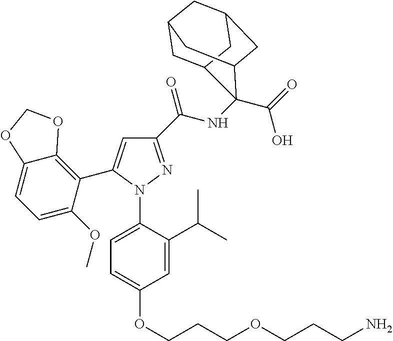 | 686 [M + H]⁺ |
| 360 | (5r,7r)-2-(1-(4-(9-(3- Aminopropyl)-2-oxo- 3,9- diazaspiro[5.5]undecan- 3-yl)-2- isopropylphenyl)-5-(5- methoxybenzo[d][1,3] dioxol-4-yl)-1H- pyrazole-3- carboxamido)adamantane- 2-carboxylic acid | 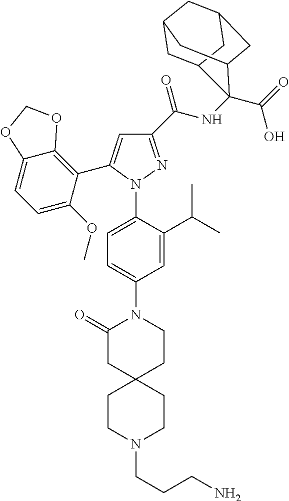 [SMILES: COc1ccc2c(c1-c1cc(C(=O)NC3(C(=O)O)C4CC5CC(C4)CC3C5)nn1-c1ccc(N3CCC4(CCN(CCCN)CC4)CC3=O)cc1C(C)C)OCO2] | 782 [M + H]⁺ |
| 361 | (5r,7r)-2-(1-(4-(5-(3- Aminopropyl)(methyl) amino)propyl)-1,3,4- isopropylphenyl)-5-(5- methoxybenzo[d][1,3] oxadiazol-2-yl)-2- dioxol-4-yl)-1H- pyrazole-3- carboxamido)adamant ane-2-carboxylic acid | 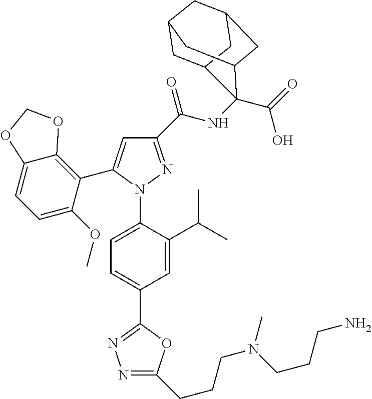 [SMILES: COc1ccc2c(c1-c1cc(C(=O)NC3(C(=O)O)C4CC5CC(C4)CC3C5)nn1-c1ccc(-c3nnc(CCCN(C)CCCN)o3)cc1C(C)C)OCO2] | 755 [M + H]⁺ |

### Preparation 400

### (5r,7r)-2-(1-(4-(7-(3-Aminopropyl)-2,7-diazaspiro[3.5]nonan-2-yl)-2-isopropylphenyl)-5-(5-methoxybenzo[d][1,3]dioxol-4-yl)-1H-pyrazole-3-carboxamido)adamantane-2-carboxylic acid

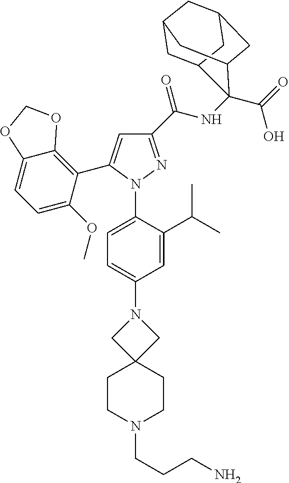 [SMILES: COc1ccc2c(c1-c1cc(C(=O)NC3(C(=O)O)C4CC5CC(C4)CC3C5)nn1-c1ccc(N3CC4(CCN(CCCN)CC4)C3)cc1C(C)C)OCO2]

To a soln of tert-butyl(2r,5S,7S)-2-((M)-1-(4-(7-(3-((tert-butoxycarbonyl)amino)propyl)-2,7-diazaspiro[3.5]nonan-2-yl)-2-isopropylphenyl)-5-(5-methoxybenzo[d][1,3]dioxol-4-yl)-1H-pyrazole-3-carboxamido)adamantane-2-carboxylate (121 mg, 0.135 mmol) in ACN (3 mL) was added pTsOH. H₂O (51.4 mg, 0.27 mmol) and stirred at RT for 15 hours. An additional pTsOH·H₂O (51.4 mg, 0.27 mmol) was added and stirred at RT for 7 hours. The mixture was dissolved with CHCl₃/IPA (3:1) and washed with sat soln of NaHCO₃. The organic layer was separated, and the aq layer extracted with CHCl₃/IPA (3:1) (×2). The combined organic layers were washed with sat soln of NaHCO₃, concentrated under reduced pressure and dried under a stream of N₂ to afford title compound (72 mg, 72%) as a yellow semisolid. MS ES+ m/z 739 [M+H]⁺.

### Preparation 401

### (5r,7r)-2-(1-(4-(4-(2-Aminoethoxy)piperidine-1-carboxamido)-2-isopropylphenyl)-5-(5-methoxybenzo[d][1,3]dioxol-4-yl)-1H-pyrazole-3-carboxamido)adamantane-2-carboxylic acid

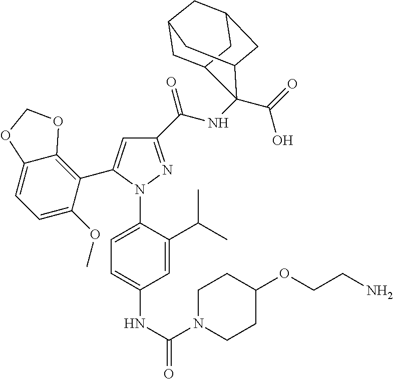 [SMILES: COc1ccc2c(c1-c1cc(C(=O)NC3(C(=O)O)C4CC5CC(C4)CC3C5)nn1-c1ccc(NC(=O)N3CCC(OCCN)CC3)cc1C(C)C)OCO2]

A soln of tert-butyl (1R,3S,5r,7r)-2-(1-(4-(4-(2-((tert-butoxycarbonyl)amino) ethoxy)piperidine-1-carboxamido)-2-isopropylphenyl)-5-(5-methoxybenzo[d][1,3]dioxol-4-yl)-1H-pyrazole-3-carboxamido)adamantane-2-carboxylate (143 mg, 0.16 mmol) in DCM (1 mL) was treated with HCl in dioxane (0.2 mL, 4 M, 0.8 mmol). The mixture was stirred for 18 h and concentrated under a steam of N₂ to provide title compound (120 mg, 100%) as a yellow oil. MS ES+ m/z 741 [M+H]⁺.

The following compounds were prepared in a manner essentially analogous to the preparation method of (5r,7r)-2-(1-(4-(4-(2-Aminoethoxy)piperidine-1-carboxamido)-2-isopropylphenyl)-5-(5-methoxybenzo[d][1,3]dioxol-4-yl)-1H-pyrazole-3-carboxamido)adamantane-2-carboxylic acid using the appropriate reagents, adjusting temperature, adjusting reaction time to determine completion of the reaction, and adjusting the purification system as appropriate.

TABLE 56

| | | | MS |
|---|---|---|---|
| Prep | | | ES+ |
| # | Chemical Name | Structure | m/z |
| 362 | (5r,7r)-2-(1-(4-(3-(3-((3- aminopropyl)(methyl) amino)propyl)ureido)- 2-isopropylphenyl)-5-(5- methoxybenzo[d][1,3] dioxol-4-yl)-1H- pyrazole-3- carboxamido) adamantane- 2-carboxylic acid(5r,7r)-2-(1-(4-(3- (3-((3- aminopropyl)(methyl) amino)propyl)ureido)- 2-isopropylphenyl)-5- (5- methoxybenzo[d][1,3] dioxol-4-yl)-1H- pyrazole-3- carboxamido) adamantane-2- carboxylic acid | 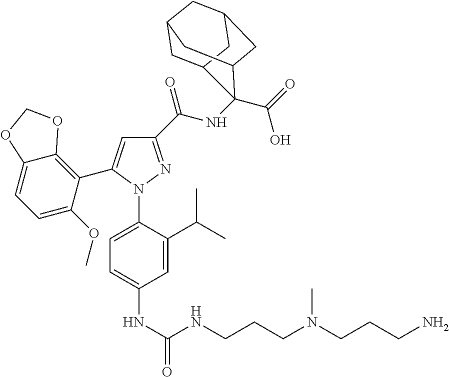 [SMILES: COc1ccc2c(c1-c1cc(C(=O)NC3(C(=O)O)C4CC5CC(C4)CC3C5)nn1-c1ccc(NC(=O)NCCCN(C)CCCN)cc1C(C)C)OCO2] | 745 [M + H]⁺ |
| 363 | (5r,7r)-2-(1-(4-(2-(4- (3- Aminopropyl)piperazin- 1-yl)ethoxy)-2- isopropylphenyl)-5-(5- methoxybenzo[d][1,3] dioxol-4-yl)-1H- pyrazole-3- carboxamido)adamantane- 2-carboxylic acid | 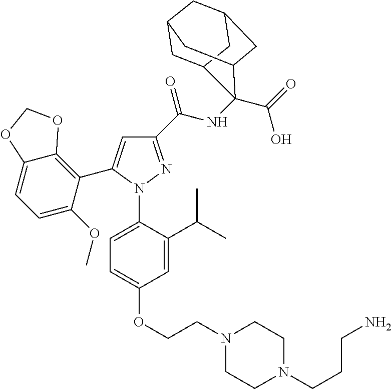 [SMILES: COc1ccc2c(c1-c1cc(C(=O)NC3(C(=O)O)C4CC5CC(C4)CC3C5)nn1-c1ccc(OCCN3CCN(CCCN)CC3)cc1C(C)C)OCO2] | 744 [M + H]⁺ |

### Preparation 402

### tert-Butyl (5R,7R)-2-(1-(4-((1R,4R)-5-(3-aminopropyl)-2,5-diazabicyclo[2.2.1]heptan-2-yl)-2-isopropylphenyl)-5-(5-methoxybenzo[d][1,3]dioxol-4-yl)-1H-pyrazole-3-carboxamido)adamantane-2-carboxylate

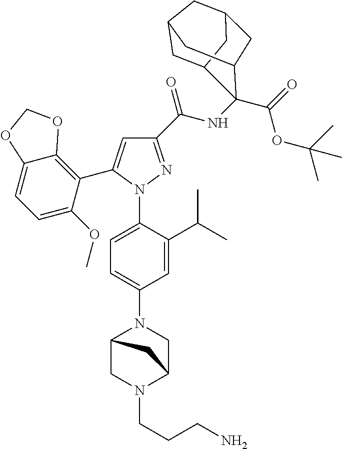 [SMILES: COc1ccc2c(c1-c1cc(C(=O)NC3(C(=O)OC(C)(C)C)C4CC5CC(C4)CC3C5)nn1-c1ccc(N3C[C@H]4C[C@@H]3CN4CCCN)cc1C(C)C)OCO2]

To a soln of tert-butyl(2r,5S,7S)-2-((M)-1-(4-((1R,4R)-5-(3-((tert-butoxycarbonyl)amino)propyl)-2,5-diazabicyclo[2.2.1]heptan-2-yl)-2-isopropylphenyl)-5-(5-methoxybenzo[d][1,3]dioxol-4-yl)-1H-pyrazole-3-carboxamido)adamantane-2-carboxylate (170 mg, 0.2 mmol) in THF (3 mL) and MeOH (0.6 mL) was added TMS (0.18 mL, 1.47 mmol). After stirring for 18 h at RT the mixture was purified by reversed-phase flash chromatography with the following conditions: column, C18; mobile phase, 10% to 100% ACN in H₂O (0.1% FA) to provide title compound (109 mg, 73%) as a white solid. MS ES+ m/z 767 [M+H]⁺.

The following compounds were prepared in a manner essentially analogous to the preparation method of tert-Butyl (5R,7R)-2-(1-(4-((1R,4R)-5-(3-aminopropyl)-2,5-diazabicyclo[2.2.1]heptan-2-yl)-2-isopropylphenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 57

| | | | MS |
|---|---|---|---|
| Prep | | | ES+ |
| # | Chemical Name | Structure | m/z |
| 364 | tert-Butyl (5S,7S)-2- (1-(4-((1S,4S)-5-(3- aminopropyl)-2,5- diazabicyclo[2.2.1] heptan-2-yl)-2- isopropylphenyl)-5-(5- methoxybenzo[d][1,3] dioxol-4-yl)-1H- pyrazole-3- carboxamido) adamantane- 2-carboxylate | 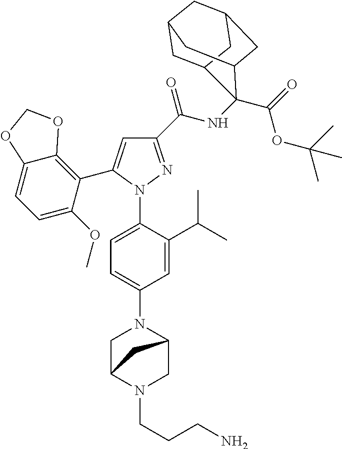 [SMILES: COc1ccc2c(c1-c1cc(C(=O)NC3(C(=O)OC(C)(C)C)C4CC5CC(C4)CC3C5)nn1-c1ccc(N3C[C@@H]4C[C@H]3CN4CCCN)cc1C(C)C)OCO2] | 768 [M + H]⁺ |
| 365 | tert-Butyl (5r,7r)-2-(1- (4-(7-(3-((3- aminopropyl)(methyl) amino)propoxy) quinolin-3-yl)-2- isopropylphenyl)-5-(5- methoxybenzo[d][1,3] dioxol-4-yl)-1H- pyrazole-3- carboxamido) adamantane- 2-carboxylate | 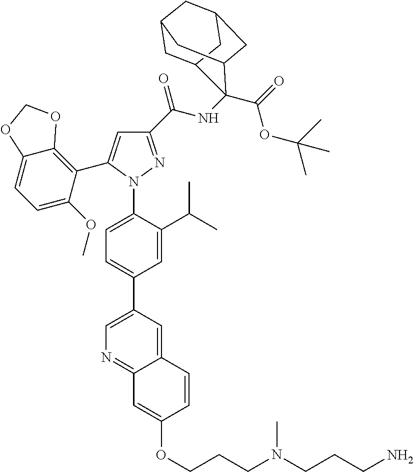 [SMILES: COc1ccc2c(c1-c1cc(C(=O)NC3(C(=O)OC(C)(C)C)C4CC5CC(C4)CC3C5)nn1-c1ccc(-c3cnc4cc(OCCCN(C)CCCN)ccc4c3)cc1C(C)C)OCO2] | 886 [M + H]⁺ |
| 366 | tert-Butyl (5r,7r)-2-(1- (4-(2-(1-((S)-2-amino- 5-(tert-butoxy)-5- oxopentanoyl) piperidin- 4-yl)ethoxy)-2- isopropylphenyl)-5-(5- methoxybenzo[d][1,3] dioxol-4-yl)-1H- pyrazole-3- carboxamido) adamantane- 2-carboxylate | 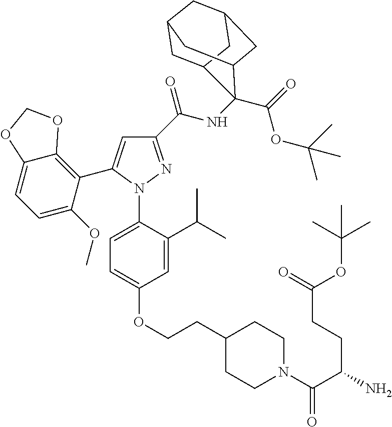 [SMILES: COc1ccc2c(c1-c1cc(C(=O)NC3(C(=O)OC(C)(C)C)C4CC5CC(C4)CC3C5)nn1-c1ccc(OCCC3CCN(C(=O)[C@@H](N)CCC(=O)OC(C)(C)C)CC3)cc1C(C)C)OCO2] | 927 [M + H]⁺ |

### Preparation 270

### tert-Butyl (5r,7r)-2-(1-(4-(2-((2-aminoethyl)(methyl)amino)ethoxy)-2-isopropylphenyl)-5-(5-methoxybenzo[d][1,3]dioxol-4-yl)-1H-pyrazole-3-carboxamido)adamantane-2-carboxylate

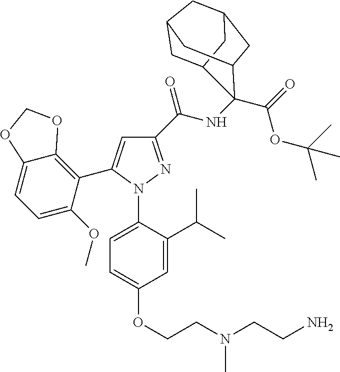 [SMILES: COc1ccc2c(c1-c1cc(C(=O)NC3(C(=O)OC(C)(C)C)C4CC5CC(C4)CC3C5)nn1-c1ccc(OCCN(C)CCN)cc1C(C)C)OCO2]

A soln of tert-butyl (5r,7r)-2-(1-(4-(2-((2-(1,3-dioxoisoindolin-2-yl)ethyl)(methyl)amino)ethoxy)-2-isopropylphenyl)-5-(5-methoxybenzo[d][1,3]dioxol-4-yl)-1H-pyrazole-3-carboxamido)adamantane-2-carboxylate (53.2 mg, 0.062 μmol) in EtOH (1 mL) was treated with N₂H₄·H₂O (0.024 mL, 0.495 mmol). After heating at 80° C. for 16 h the mixture was heated at 95° C. for 6 hours. The mixture was concentrated to afford crude title compound (45 mg, 100%). MS ES+ m/z 730 [M+H]⁺.

The following compounds were prepared in a manner essentially analogous to the preparation method of tert-Butyl (5r,7r)-2-(1-(4-(2-((2-aminoethyl)(methyl)amino)ethoxy)-2-isopropylphenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 44

| | | | MS |
|---|---|---|---|
| Prep | | | ES+ |
| # | Chemical Name | Structure | m/z |
| 271 | tert-Butyl (5r,7r)-2-(1- (4-(((2-((2- aminoethyl)(methyl)a- mino)ethoxy)carbonyl) amino)-2- isopropylphenyl)-5-(5- methoxybenzo[d][1,3] dioxol-4-yl)-1H- pyrazole-3- carboxamido)adamantane- 2-carboxylate | 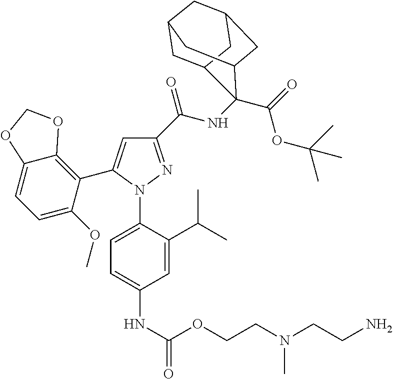 [SMILES: COc1ccc2c(c1-c1cc(C(=O)NC3(C(=O)OC(C)(C)C)C4CC5CC(C4)CC3C5)nn1-c1ccc(NC(=O)OCCN(C)CCN)cc1C(C)C)OCO2] | 774 [M + H]⁺ |
| 272 | (5r,7r)-2-(1-(4-(1-(3- (3- Aminopropoxy)propyl)- 1H-1,2,3-triazol-4- yl)-2- isopropylphenyl)-5-(5- methoxybenzo[d][1,3] dioxol-4-yl)-1H- pyrazole-3- carboxamido)adamantane- 2-carboxylic acid | 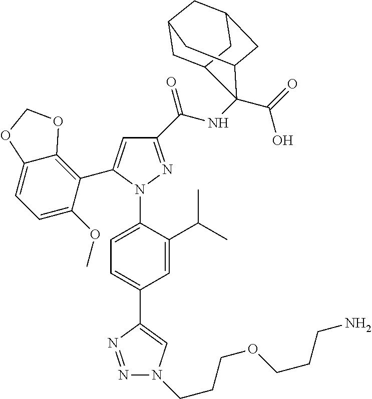 | 797 [M + H]⁺ |
| 367 | tert-Butyl (5r,7r)-2-(1- (4′-(3-((3- aminopropyl)(methyl) amino)propoxy)-3- isopropyl-[1,1′- biphenyl]-4-yl)-5-(5- methoxybenzo[d][1,3] dioxol-4-yl)-1H- pyrazole-3- carboxamido)adamantane- 2-carboxylate | 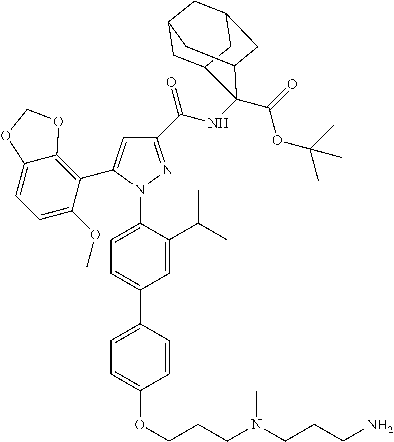 [SMILES: COc1ccc2c(c1-c1cc(C(=O)NC3(C(=O)OC(C)(C)C)C4CC5CC(C4)CC3C5)nn1-c1ccc(-c3ccc(OCCCN(C)CCCN)cc3)cc1C(C)C)OCO2] | 835 [M + H]⁺ |

### Preparation 403

### tert-Butyl (5r,7r)-2-(1-(4-(4-(3-((3-aminopropyl)(methyl)amino)propyl)piperazin-1-yl)-2-isopropylphenyl)-5-(5-methoxybenzo[d][1,3]dioxol-4-yl)-1H-pyrazole-3-carboxamido)adamantane-2-carboxylate

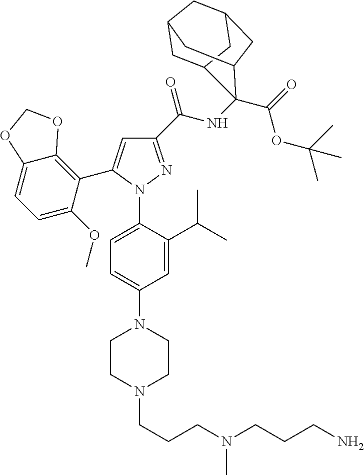 [SMILES: COc1ccc2c(c1-c1cc(C(=O)NC3(C(=O)OC(C)(C)C)C4CC5CC(C4)CC3C5)nn1-c1ccc(N3CCN(CCCN(C)CCCN)CC3)cc1C(C)C)OCO2]

A soln of tert-butyl(2r,5S,7S)-2-((M)-1-(4-(4-(3-((3-(((benzyloxy)carbonyl)amino)propyl)(methyl)amino)propyl)piperazin-1-yl)-2-isopropylphenyl)-5-(5-methoxybenzo[d][1,3]dioxol-4-yl)-1H-pyrazole-3-carboxamido)adamantane-2-carboxylate (154 mg, 0.16 mmol) in EtOH (5 mL) was degassed with argon for 3 minutes, and treated Pd/C (17.1 mg, 20% wt, 0.032 mmol). The mixture was degassed with argon and purged with H₂. The mixture was stirred at RT under a H₂ balloon for 2 hours. Additional Pd/C (34 mg, 20% wt, 0.064 mol) was added and the mixture purged with H₂ (×3) and stirred for 2 h under a H₂ balloon. The mixture was filtered through diatomaceous earth and filter cake washed with EtOH. The combined filtrate was concentrated to afford title compound (120 mg, 91%) as an orange oil. MS ES+ m/z 827 [M+H]⁺.

### Preparation 273

### tert-Butyl (5r,7r)-2-(1-(4-(4-hydroxybutyl)-2-isopropylphenyl)-5-(5-methoxybenzo[d][1,3]dioxol-4-yl)-1H-pyrazole-3-carboxamido)adamantane-2-carboxylate

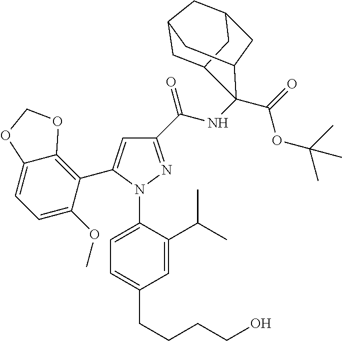 [SMILES: COc1ccc2c(c1-c1cc(C(=O)NC3(C(=O)OC(C)(C)C)C4CC5CC(C4)CC3C5)nn1-c1ccc(CCCCO)cc1C(C)C)OCO2]

A soln of PtO₂ (32 mg, 0.141 mmol) in EtOH (5 mL) at RT was treated with tert-butyl 2-{1-[4-(4-hydroxybut-1-yn-1-yl)-2-isopropylphenyl]-5-(5-methoxy-2H-1,3-benzodioxol-4-yl)pyrazole-3-amido}adamantane-2-carboxylate (480 mg, 0.704 mmol) in portions. After stirring at RT for 2 h under H₂, the mixture was filtered, and the filter cake was washed with EtOH (3×10 mL). The filtrate was concentrated to afford the title crude product (400 mg). MS ES+ m/z 686 [M+H]⁺.

### Preparation 274

### tert-Butyl (5r,7r)-2-(1-(2-isopropyl-4-(4-oxobutyl)phenyl)-5-(5-methoxybenzo[d][1,3]dioxol-4-yl)-1H-pyrazole-3-carboxamido)adamantane-2-carboxylate

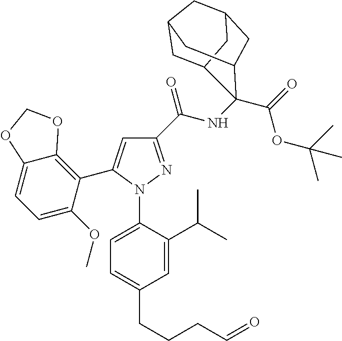 [SMILES: COc1ccc2c(c1-c1cc(C(=O)NC3(C(=O)OC(C)(C)C)C4CC5CC(C4)CC3C5)nn1-c1ccc(CCCC=O)cc1C(C)C)OCO2]

A soln of tert-butyl 2-{1-[4-(4-hydroxybutyl)-2-isopropylphenyl]-5-(5-methoxy-2H-1,3-benzodioxol-4-yl)pyrazole-3-amido}adamantane-2-carboxylate (100 mg, 0.146 mmol) in DCM (5 mL) was treated with DMP (124 mg, 0.292 mmol) in portions at 0° C. After stirring at 50° C. for 2 h, the mixture was filtered, and the filter cake was washed with DCM (3×10 mL). The filtrate was concentrated to provide crude title compound (260 mg). MS ES+ m/z 684 [M+H]⁺.

### Preparation 275

### tert-Butyl (5r,7r)-2-(1-(2-isopropyl-4-(4-(methyl(3-(methylamino)propyl)amino)butyl)phenyl)-5-(5-methoxybenzo[d][1,3]dioxol-4-yl)-1H-pyrazole-3-carboxamido)adamantane-2-carboxylate

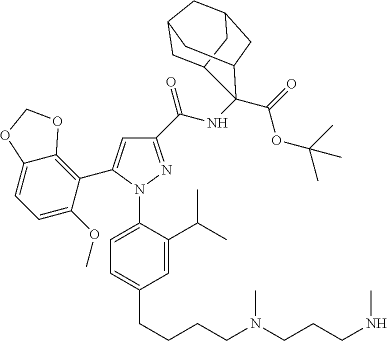 [SMILES: CNCCCN(C)CCCCc1ccc(-n2nc(C(=O)NC3(C(=O)OC(C)(C)C)C4CC5CC(C4)CC3C5)cc2-c2c(OC)ccc3c2OCO3)c(C(C)C)c1]

A soln of tert-butyl 2-{1-[2-isopropyl-4-(4-oxobutyl)phenyl]-5-(5-methoxy-2H-1,3-benzodioxol-4-yl)pyrazole-3-amido}adamantane-2-carboxylate (250 mg, 0.366 mmol) in MeOH (10 mL) was treated with methyl[3-(methylamino)propyl]amine (75 mg, 0.732 mmol) at RT. After stirring for 30 min the mixture was treated with STAB (387 mg, 1.828 mmol) at 0° C. After stirring at RT for 2 h, the mixture was purified by reversed-phase flash chromatography with the following conditions: column, C18; mobile phase, 10% to 50% ACN in MeOH (0.1% FA) to provide title compound (250 mg, 50%) as a light yellow solid. MS ES+ m/z 771 [M+H]⁺.

### Preparation 276

### tert-Butyl (5r, 7r)-2-(1-(4-(4-((S)-6-amino-2-(4-((4-(4-iodophenyl)butanamido)methyl)benzamido)hexanamido)butanamido)-2-isopropylphenyl)-5-(5-methoxybenzo[d][1,3]dioxol-4-yl)-1H-pyrazole-3-carboxamido)adamantane-2-carboxylate

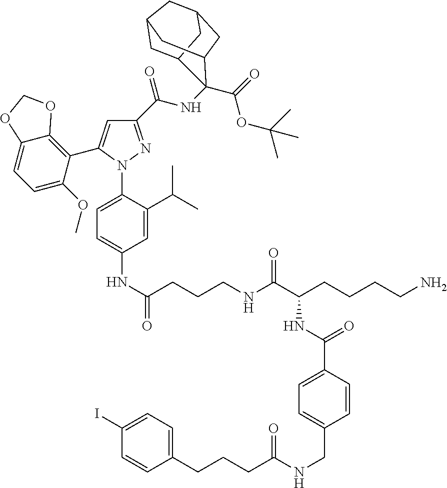 [SMILES: COc1ccc2c(c1-c1cc(C(=O)NC3(C(=O)OC(C)(C)C)C4CC5CC(C4)CC3C5)nn1-c1ccc(NC(=O)CCCNC(=O)[C@H](CCCCN)NC(=O)c3ccc(CNC(=O)CCCc4ccc(I)cc4)cc3)cc1C(C)C)OCO2]

A soln of tert-butyl 2-[1-(4-{4-[(2S)-6-{[(9H-fluoren-9-ylmethoxy)carbonyl]amino}-2-[(4-{[4-(4-iodophenyl)butanamido]methyl}phenyl)formamido]hexanamido]butanamido}-2-isopropylphenyl)-5-(5-methoxy-2H-1,3-benzodioxol-4-yl)pyrazole-3-amido]adamantane-2-carboxylate (120 mg, 0.082 mmol) and DBU (37 mg, 0.246 mmol) in DCM (4 mL) was stirred at RT under N₂. After 2 h the mixture was concentrated and purified by reversed-phase flash chromatography with the following conditions: column, C18; mobile phase, 55% to 60% ACN in H₂O (0.1% HCl) to provide title compound (91 mg, 89%). MS ES+ m/z 1248 [M+H]⁺.

The following compounds were prepared in a manner essentially analogous to the preparation method of tert-Butyl (5r, 7r)-2-(1-(4-(4-((S)-6-amino-2-(4-((4-(4-iodophenyl)butanamido)methyl)benzamido)hexanamido)butanamido)-2-isopropylphenyl)-5-(5-methoxybenzo[d][1,3]dioxol-4-yl)-1H-pyrazole-3-carboxamido)adamantane-2-carboxylate using the appropriate reagents, adjusting temperature, adjusting reaction time to determine completion of the reaction, and adjusting the purification system as appropriate.

TABLE 45

| Prep | | | MS ES+ |
|---|---|---|---|
| # | Chemical Name | Structure | m/z |
| 277 | tert-Butyl (5r,7r)-2- (1-(4-(3-(3-((S)-6- amino-2-(4-((4-(4- iodophenyl)butanami- do)methyl)benzamido) hexanamido)propyl)- 3-(3- (dimethylamino)pro- pyl)ureido)-2- isopropylphenyl)-5- (5- methoxybenzo[d][1,3] dioxol-4-yl)-1H- pyrazole-3- carboxamido)adaman- tane-2-carboxylate | 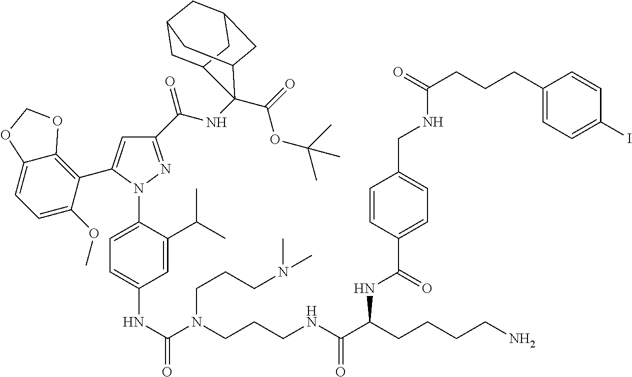 [SMILES: COc1ccc2c(c1-c1cc(C(=O)NC3(C(=O)OC(C)(C)C)C4CC5CC(C4)CC3C5)nn1-c1ccc(NC(=O)N(CCCNC(=O)[C@H](CCCCN)NC(=O)c3ccc(CNC(=O)CCCc4ccc(I)cc4)cc3)CCCN(C)C)cc1C(C)C)OCO2] | 675 [M/2 + H]⁺ |
| 278 | (5r,7r)-2-(1-(4-((3-(6- Amino-2-(4-((4-(4- iodophenyl)butanami- do)methyl)benzamido) hexanamido)propyl)(3- (dimethylamino)pro- pyl)amino)-2- isopropylphenyl)-5- (5- methoxybenzo[d][1,3] dioxol-4-yl)-1H- pyrazole-3- carboxamido)adaman- tane-2-carboxylic acid, Isomer 1 | 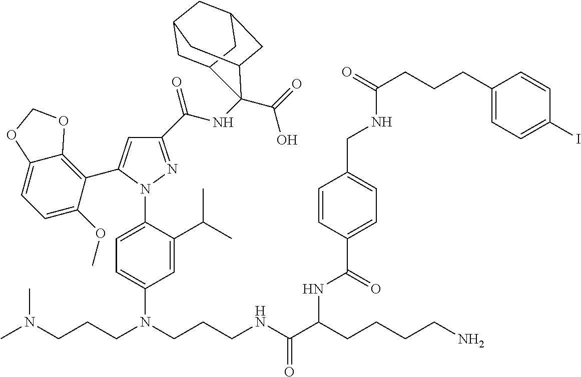 [SMILES: COc1ccc2c(c1-c1cc(C(=O)NC3(C(=O)O)C4CC5CC(C4)CC3C5)nn1-c1ccc(N(CCCNC(=O)C(CCCCN)NC(=O)c3ccc(CNC(=O)CCCc4ccc(I)cc4)cc3)CCCN(C)C)cc1C(C)C)OCO2] | 625 [M/2 + H]⁺ |
| 279 | tert-Butyl (5r,7r)-2- (1-(4-((3-(6-amino-2- (4-((4-(4- iodophenyl)butanami- do)methyl)benzamido) hexanamido)propyl)(3- (dimethylamino)propyl) amino)-2- isopropylphenyl)-5- (5- methoxybenzo[d][1,3] dioxol-4-yl)-1H- pyrazole-3- carboxamido)adaman- tane-2-carboxylate, Isomer 2 | 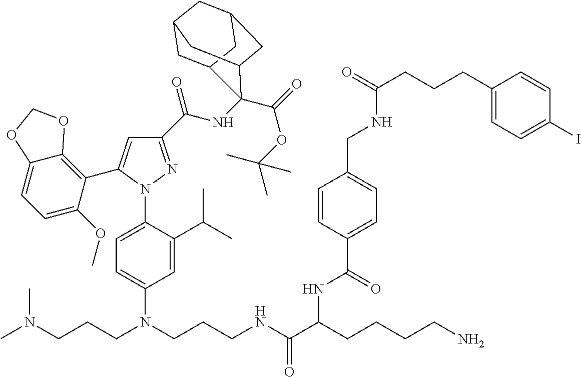 [SMILES: COc1ccc2c(c1-c1cc(C(=O)NC3(C(=O)OC(C)(C)C)C4CC5CC(C4)CC3C5)nn1-c1ccc(N(CCCNC(=O)C(CCCCN)NC(=O)c3ccc(CNC(=O)CCCc4ccc(I)cc4)cc3)CCCN(C)C)cc1C(C)C)OCO2] | 1305 [M + H]⁺ |

### Preparation 73

### (5r,7r)-2-(1-(2-Isopropyl-4-(3-(methyl(2-(2-(4,7,10-tris(2-(tert-butoxy)-2-oxoethyl)-1,4,7,10-tetraazacyclododecan-1-yl)acetamido)ethyl)amino)propoxy)phenyl)-5-(5-methoxybenzo[d][1,3]dioxol-4-yl)-1H-pyrazole-3-carboxamido)adamantane-2-carboxylic acid

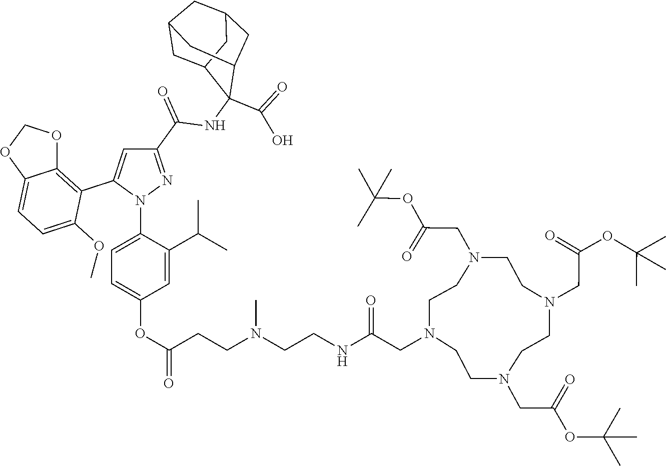 [SMILES: COc1ccc2c(c1-c1cc(C(=O)NC3(C(=O)O)C4CC5CC(C4)CC3C5)nn1-c1ccc(OC(=O)CCN(C)CCNC(=O)CN3CCN(CC(=O)OC(C)(C)C)CCN(CC(=O)OC(C)(C)C)CCN(CC(=O)OC(C)(C)C)CC3)cc1C(C)C)OCO2]

A soln of tri-tert-butyl 2,2′,2″-(10-(2-((2,5-dioxopyrrolidin-1-yl)oxy)-2-oxoethyl)-1,4,7,10-tetraazacyclododecane-1,4,7-triyl)triacetate (102 mg, 0.153 mmol) and (5r,7r)-2-(1-(4-(3-((2-aminoethyl)(methyl)amino)propoxy)-2-isopropylphenyl)-5-(5-methoxybenzo[d][1,3]dioxol-4-yl)-1H-pyrazole-3-carboxamido)adamantane-2-carboxylic acid (70 mg, 0.102 mmol) in DMF (1 mL) was treated with DIEA (78.9 mg, 0.611 mmol). After stirring at RT for 0.5 h the mixture was concentrated to afford crude title compound (126 mg, 100%). MS ES+ m/z 1243 [M+H]⁺.

The following compounds were prepared in a manner essentially analogous to the preparation method of (5r,7r)-2-(1-(2-Isopropyl-4-(3-(methyl(2-(2-(4,7,10-tris(2-(tert-butoxy)-2-oxoethyl)-1,4,7,10-tetraazacyclododecan-1-yl)acetamido)ethyl)amino)propoxy)phenyl)-5-(5-methoxybenzo[d][1,3]dioxol-4-yl)-1H-pyrazole-3-carboxamido)adamantane-2-carboxylic acid using the appropriate reagents, adjusting temperature, adjusting reaction time to determine completion of the reaction, and adjusting the purification system as appropriate.

TABLE 17

| Prep | | | MS ES+ |
|---|---|---|---|
| # | Chemical Name | Structure | m/z |
| 74 | tri-tert-Butyl 2,2′,2″- (10-(2-((3-((3-((N-(4- (3-(((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)sulfa- moyl)(methyl)amino) propyl)(methyl)amino) propyl)(methyl)ami- no)-2-oxoethyl)- 1,4,7,10- tetraazacyclododecane- 1,4,7-triyl)triacetate | 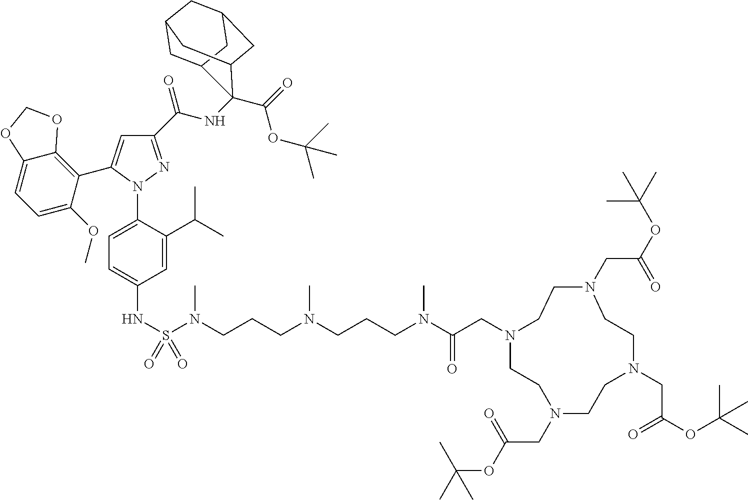 [SMILES: COc1ccc2c(c1-c1cc(C(=O)NC3(C(=O)OC(C)(C)C)C4CC5CC(C4)CC3C5)nn1-c1ccc(NS(=O)(=O)N(C)CCCN(C)CCCN(C)C(=O)CN3CCN(CC(=O)OC(C)(C)C)CCN(CC(=O)OC(C)(C)C)CCN(CC(=O)OC(C)(C)C)CC3)cc1C(C)C)OCO2] | 1419 [M + H]⁺ |
| 75 | tri-tert-Butyl 2,2′,2″- (10-(2-((3-((3-((4-(3- (((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)amino) propyl)(methyl)ami- no)propyl)amino)-2- oxoethyl)-1,4,7,10- tetraazacyclododecane- 1,4,7-triyl)triacetate | 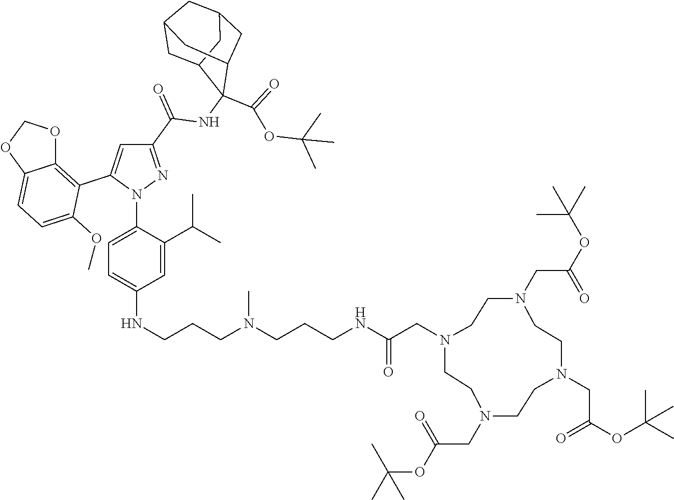 [SMILES: COc1ccc2c(c1-c1cc(C(=O)NC3(C(=O)OC(C)(C)C)C4CC5CC(C4)CC3C5)nn1-c1ccc(NCCCN(C)CCCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 1312 [M + H]⁺ |
| 76 | (5r,7r)-2-(1-(2- Isopropyl-4-(1-(3- (methyl(3-(N-methyl- 2-(4,7,10-tris(2-(tert- butoxy)-2-oxoethyl)- 1,4,7,10- tetraazacyclododecan- 1- yl)acetamido)propyl) amino)propyl)-1H- 1,2,3-triazol-4- yl)phenyl)-5-(5- methoxybenzo[d][1,3] dioxol-4-yl)-1H- pyrazole-3- carboxamido)adaman- tane-2-carboxylic acid | 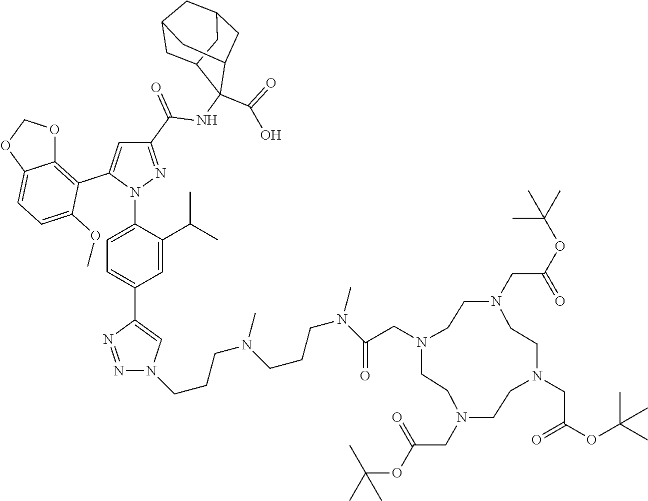 [SMILES: COc1ccc2c(c1-c1cc(C(=O)NC3(C(=O)O)C4CC5CC(C4)CC3C5)nn1-c1ccc(-c3cn(CCCN(C)CCCN(C)C(=O)CN4CCN(CC(=O)OC(C)(C)C)CCN(CC(=O)OC(C)(C)C)CCN(CC(=O)OC(C)(C)C)CC4)nn3)cc1C(C)C)OCO2] | 1323 [M + H]⁺ |
| 280 | (5r,7r)-2-(1-(2- Isopropyl-4-(3- (methyl(3-(2-(4,7,10- tris(2-(tert-butoxy)-2- oxoethyl)-1,4,7,10- tetraazacyclododecan- 1- yl)acetamido)propyl) amino)propoxy)phenyl)- 5-(5- methoxybenzo[d][1,3] dioxol-4-yl)-1H- pyrazole-3- carboxamido)adaman- tane-2-carboxylic acid | 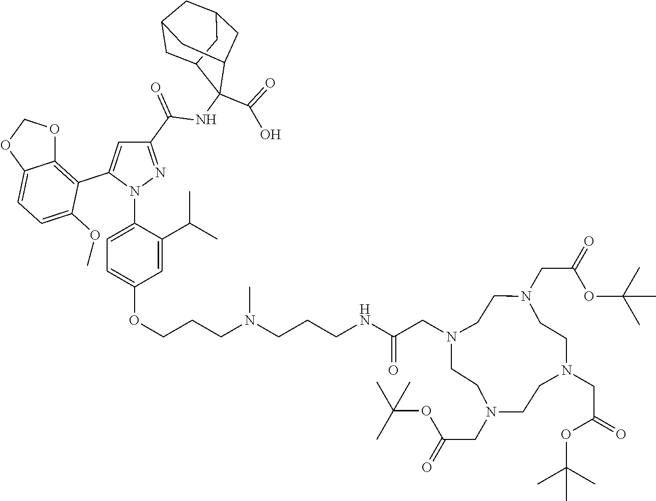 [SMILES: COc1ccc2c(c1-c1cc(C(=O)NC3(C(=O)O)C4CC5CC(C4)CC3C5)nn1-c1ccc(OCCCN(C)CCCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 1257 [M + H]⁺ |
| 281 | (5r,7r)-2-(1-(2- Isopropyl-4-(2-oxo-1- (4,7,10-tris(2-(tert- butoxy)-2-oxoethyl)- 1,4,7,10- tetraazacyclododecan- 1-yl)-6,9,12,15- tetraoxa-3- azaheptadecan-17- amido)phenyl)-5-(5- methoxybenzo[d][1,3] dioxol-4-yl)-1H- pyrazole-3- carboxamido)adaman- tane-2-carboxylic acid | 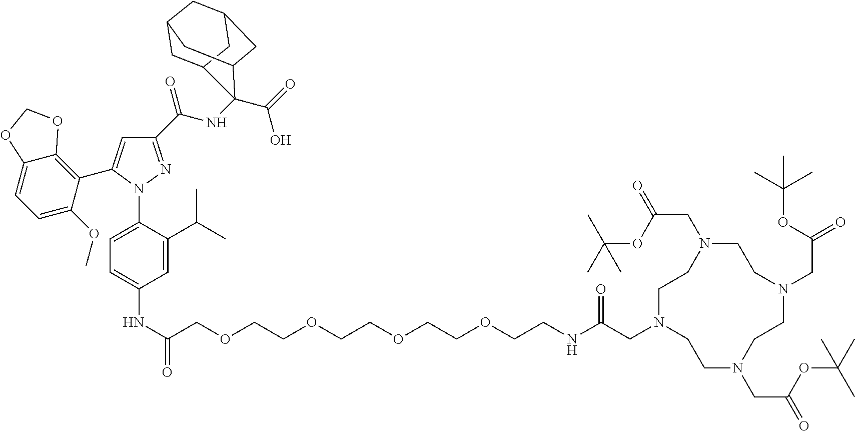 [SMILES: COc1ccc2c(c1-c1cc(C(=O)NC3(C(=O)O)C4CC5CC(C4)CC3C5)nn1-c1ccc(NC(=O)COCCOCCOCCOCCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 1361 [M + H]⁺ |
| 282 | tri-tert-butyl 2,2′,2″- (10-(2-((3-(4-(2-((4- (3-(((5r,7r)-2-(tert- Butoxycarbonyl)ada- mantan-2- yl)carbamoyl)-5-(5- methoxybenzo[d][1,3] dioxol-4-yl)-1H- pyrazol-1-yl)-3- isopropylphenyl)amino)- 2- oxoethyl)piperidin-1- yl)propyl)amino)-2- oxoethyl)-1,4,7,10- tetraazacyclododecane- 1,4,7-triyl)triacetate | 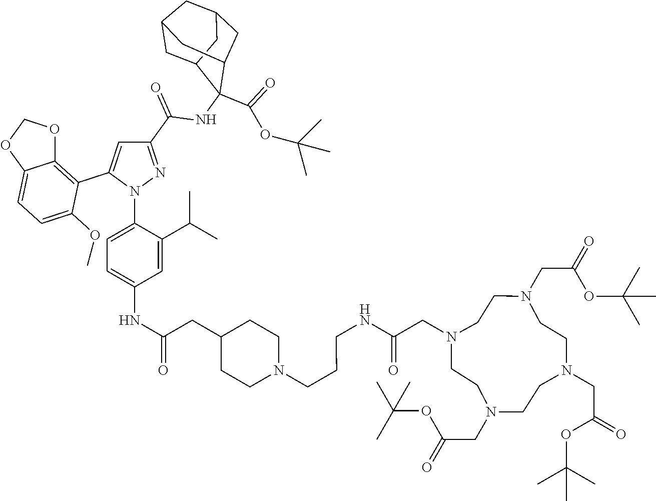 [SMILES: COc1ccc2c(c1-c1cc(C(=O)NC3(C(=O)OC(C)(C)C)C4CC5CC(C4)CC3C5)nn1-c1ccc(NC(=O)CC3CCN(CCCNC(=O)CN4CCN(CC(=O)OC(C)(C)C)CCN(CC(=O)OC(C)(C)C)CCN(CC(=O)OC(C)(C)C)CC4)CC3)cc1C(C)C)OCO2] | 1366 [M + H]⁺ |
| 283 | tri-tert-Butyl 2,2′,2″- (10-(2-((2-((2-(4-(3- (((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenoxy)eth- yl)(methyl)amino)eth- yl)amino)-2- oxoethyl)-1,4,7,10- tetraazacyclododecane- 1,4,7-triyl)triacetate | 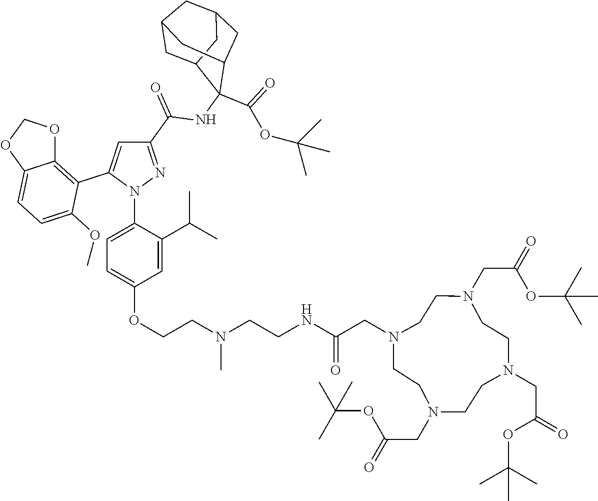 [SMILES: COc1ccc2c(c1-c1cc(C(=O)NC3(C(=O)OC(C)(C)C)C4CC5CC(C4)CC3C5)nn1-c1ccc(OCCN(C)CCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 1285 [M + H]⁺ |
| 284 | tri-tert-Butyl 2,2′,2″- (10-(2-((2-((2-(((4-(3- (((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)carba- moyl)oxy)ethyl)(meth- yl)amino)ethyl)amino)- 2-oxoethyl)- 1,4,7,10- tetraazacyclododecane- 1,4,7-triyl)triacetate | 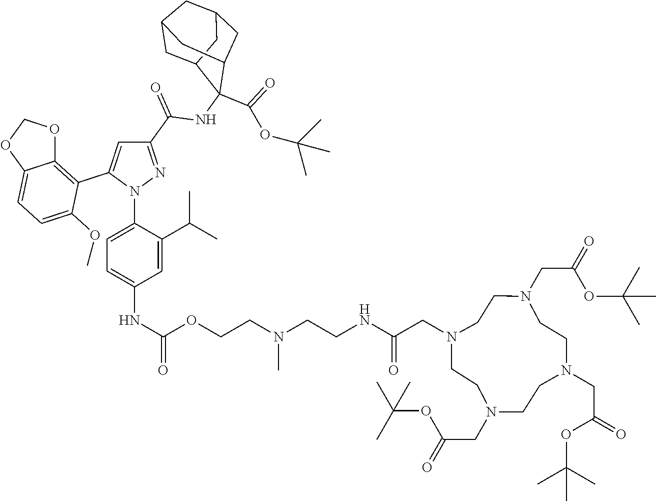 [SMILES: COc1ccc2c(c1-c1cc(C(=O)NC3(C(=O)OC(C)(C)C)C4CC5CC(C4)CC3C5)nn1-c1ccc(NC(=O)OCCN(C)CCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 1328 [M + H]⁺ |
| 285 | tri-tert-Butyl 2,2′,2″- (10-(2-((3-((3-(3-(4- (3-(((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)urei- do)propyl)(methyl)a- mino)propyl)amino)- 2-oxoethyl)-1,4,7,10- tetraazacyclododecane- 1,4,7-triyl)triacetate | 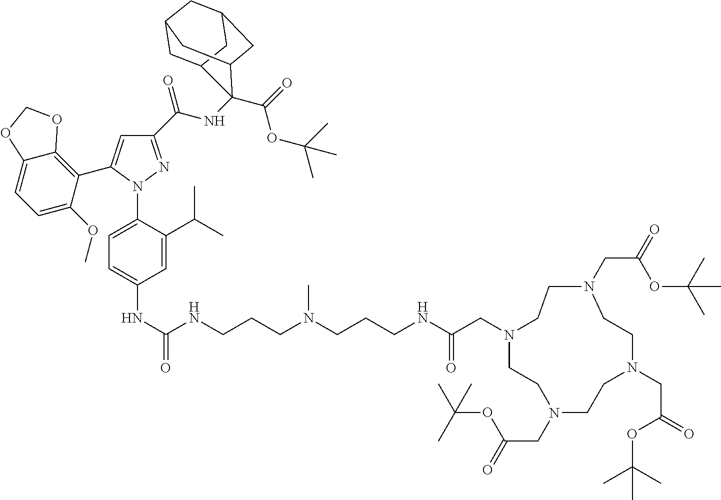 | 1300 [M + H]⁺ |
| 286 | (5r,7r)-2-(1-(2- Isopropyl-4-(1-(3- (methyl(3-(2-(4,7,10- tris(2-(tert-butoxy)-2- oxoethyl)-1,4,7,10- tetraazacyclododecan- 1- yl)acetamido)propyl) amino)propyl)-1H- 1,2,3-triazol-4- yl)phenyl)-5-(5- methoxybenzo[d][1,3] dioxol-4-yl)-1H- pyrazole-3- carboxamido)adaman- tane-2-carboxylic acid | 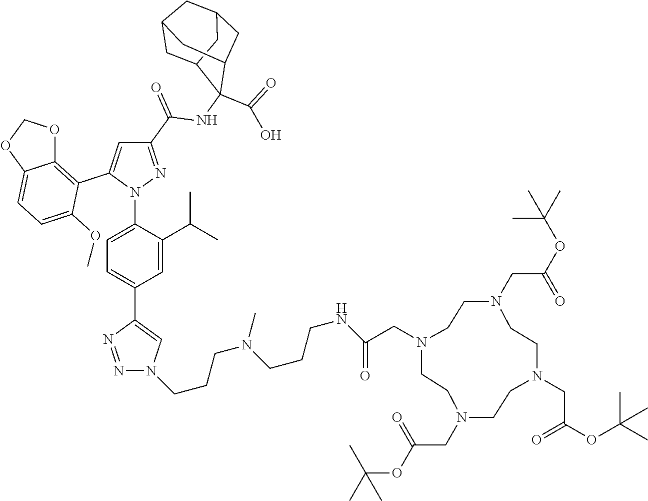 [SMILES: COc1ccc2c(c1-c1cc(C(=O)NC3(C(=O)O)C4CC5CC(C4)CC3C5)nn1-c1ccc(-c3cn(CCCN(C)CCCNC(=O)CN4CCN(CC(=O)OC(C)(C)C)CCN(CC(=O)OC(C)(C)C)CCN(CC(=O)OC(C)(C)C)CC4)nn3)cc1C(C)C)OCO2] | 1308 [M + H]⁺ |
| 287 | (5r,7r)-2-(1-(2- Isopropyl-4-(1-(3-(3- (2-(4,7,10-tris(2-(tert- butoxy)-2-oxoethyl)- 1,4,7,10- tetraazacyclododecan- 1- yl)acetamido)propoxy) propyl)-1H-1,2,3- triazol-4-yl)phenyl)- 5-(5- methoxybenzo[d][1,3] dioxol-4-yl)-1H- pyrazole-3- carboxamido)adaman- tane-2-carboxylic acid | 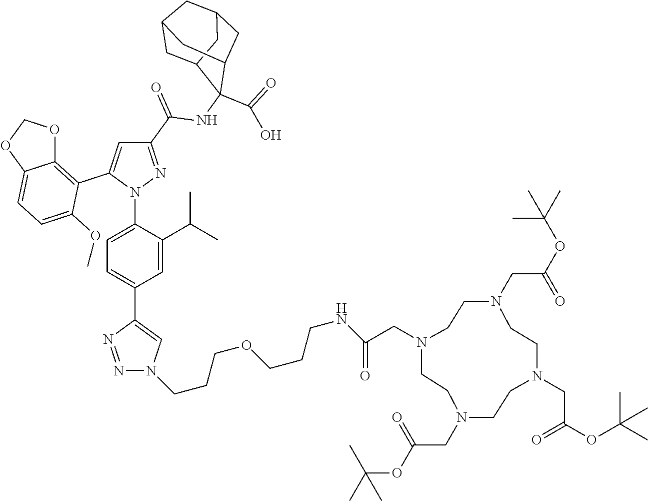 | 1351 [M + H]⁺ |
| 288 | tri-tert-Butyl 2,2′,2″- (10-(2-((3-((3-(4-(3- (ethoxycarbonyl)-5- (5- methoxybenzo[d][1,3] dioxol-4-yl)-1H- pyrazol-1-yl)-3- isopropyl-N- methylbenzamido)pro- pyl)(methyl)amino)pro- pyl)(methyl)amino)- 2-oxoethyl)-1,4,7,10- tetraazacyclododecane- 1,4,7-triyl)triacetate | 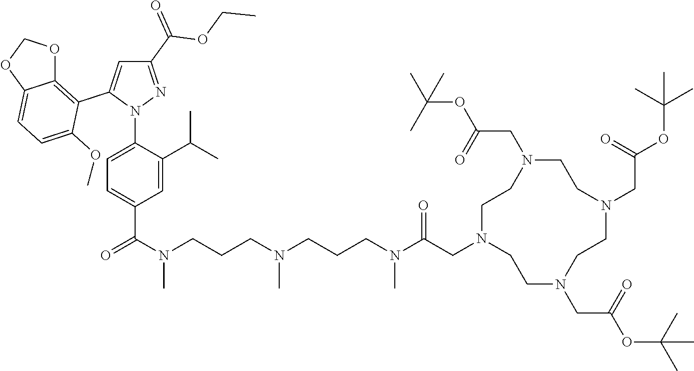 | 1177 [M + H]⁺ |
| 289 | tri-tert-Butyl 2,2′,2″- (10-(2-((3-((4-(4-(3- (((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)butyl) (methyl)amino)propyl) (methyl)amino)-2- oxoethyl)-1,4,7,10- tetraazacyclododecane- 1,4,7-triyl)triacetate | 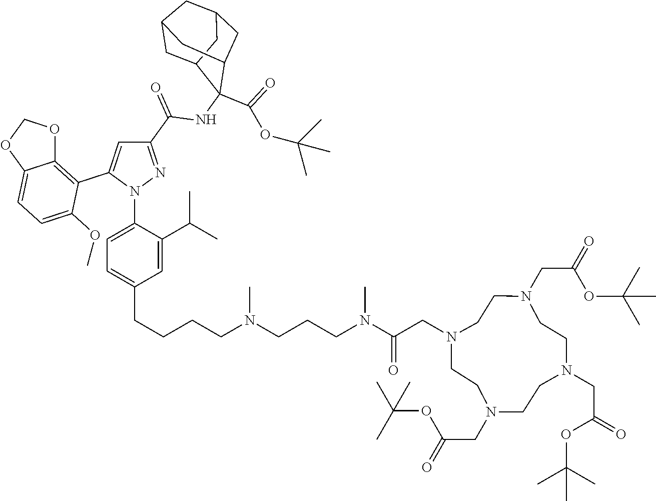 [SMILES: COc1ccc2c(c1-c1cc(C(=O)NC3(C(=O)OC(C)(C)C)C4CC5CC(C4)CC3C5)nn1-c1ccc(CCCCN(C)CCCN(C)C(=O)CN3CCN(CC(=O)OC(C)(C)C)CCN(CC(=O)OC(C)(C)C)CCN(CC(=O)OC(C)(C)C)CC3)cc1C(C)C)OCO2] | 1325 [M + H]⁺ |
| 290 | tri-tert-Butyl 2,2′,2″- (10-(2-(((5S)-6-((4- ((4-(3-(((5r,7r)-2- (tert- butoxycarbonyl)adaman- tan-2- yl)carbamoyl)-5-(5- methoxybenzo[d][1,3] dioxol-4-yl)-1H- pyrazol-1-yl)-3- isopropylphenyl)amino)- 4- oxobutyl)amino)-5- (4-((4-(4- iodophenyl)butanami- do)methyl)benzamido)- 6-oxohexyl)amino)- 2-oxoethyl)-1,4,7,10- tetraazacyclododecane- 1,4,7-triyl)triacetate | 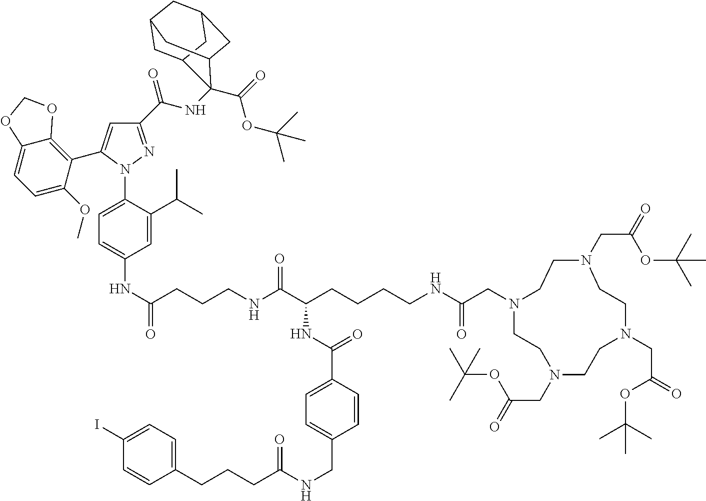 [SMILES: COc1ccc2c(c1-c1cc(C(=O)NC3(C(=O)OC(C)(C)C)C4CC5CC(C4)CC3C5)nn1-c1ccc(NC(=O)CCCNC(=O)[C@H](CCCCNC(=O)CN3CCN(CC(=O)OC(C)(C)C)CCN(CC(=O)OC(C)(C)C)CCN(CC(=O)OC(C)(C)C)CC3)NC(=O)c3ccc(CNC(=O)CCCc4ccc(I)cc4)cc3)cc1C(C)C)OCO2] | 1802/1803 [M + H]⁺ |
| 291 | tri-tert-Butyl 2,2′,2″- (10-((12S)-6-((4-(3- (((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)carba- moyl)-12-(4-((4-(4- iodophenyl)butanami- do)methyl)benzamido)- 2-methyl-11,18- dioxo-2,6,10,17- tetraazanonadecan- 19-yl)-1,4,7,10- tetraazacyclododecane- 1,4,7-triyl)triacetate | 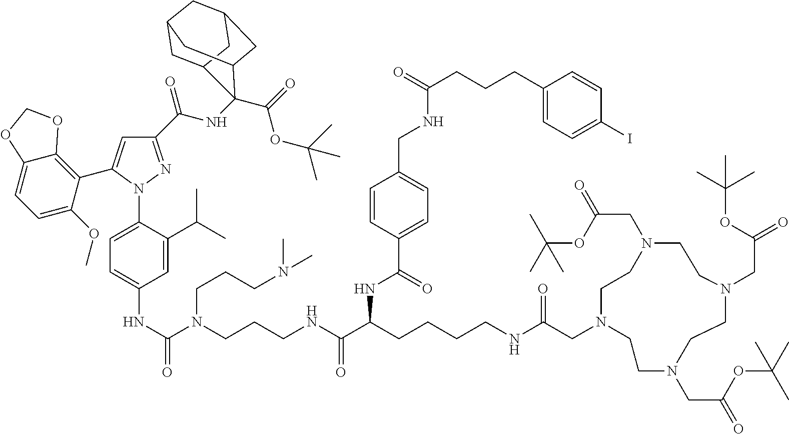 [SMILES: COc1ccc2c(c1-c1cc(C(=O)NC3(C(=O)OC(C)(C)C)C4CC5CC(C4)CC3C5)nn1-c1ccc(NC(=O)N(CCCNC(=O)[C@H](CCCCNC(=O)CN3CCN(CC(=O)OC(C)(C)C)CCN(CC(=O)OC(C)(C)C)CCN(CC(=O)OC(C)(C)C)CC3)NC(=O)c3ccc(CNC(=O)CCCc4ccc(I)cc4)cc3)CCCN(C)C)cc1C(C)C)OCO2] | 952 [M/2 + H]⁺ |
| 292 | (5r,7r)-2-(1-(4-((3- (Dimethylamino)propyl) (3-(2-(4-((4-(4- iodophenyl)butanami- do)methyl)benzamido)- 6-(2-(4,7,10-tris(2- (tert-butoxy)-2- oxoethyl)-1,4,7,10- tetraazacyclododecan- 1- yl)acetamido)hexana- mido)propyl)amino)- 2-isopropylphenyl)-5- (5- methoxybenzo[d][1,3] dioxol-4-yl)-1H- pyrazole-3- carboxamido)adaman- tane-2-carboxylic acid, Isomer 1 | 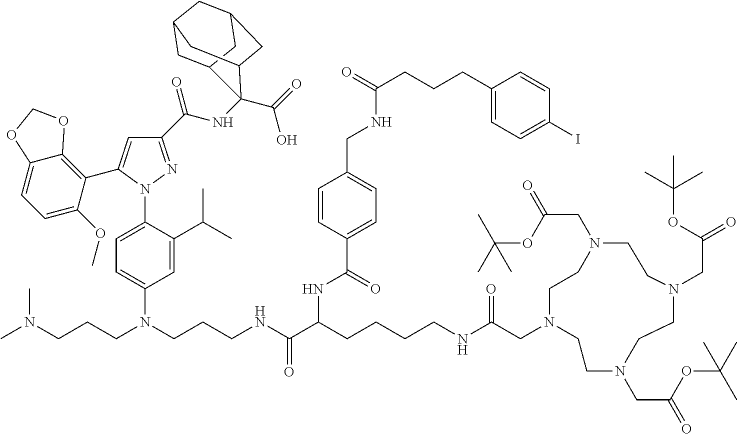 [SMILES: COc1ccc2c(c1-c1cc(C(=O)NC3(C(=O)O)C4CC5CC(C4)CC3C5)nn1-c1ccc(N(CCCNC(=O)C(CCCCNC(=O)CN3CCN(CC(=O)OC(C)(C)C)CCN(CC(=O)OC(C)(C)C)CCN(CC(=O)OC(C)(C)C)CC3)NC(=O)c3ccc(CNC(=O)CCCc4ccc(I)cc4)cc3)CCCN(C)C)cc1C(C)C)OCO2] | 902 [M/2 + H]⁺ |
| 293 | tri-tert-Butyl 2,2′,2″- (10-(6-(4-(3-(((5r,7r)- 2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)-12- (4-((4-(4- iodophenyl)butanami- do)methyl)benzamido)- 2-methyl-11,18- dioxo-2,6,10,17- tetraazanonadecan- 19-yl)-1,4,7,10- tetraazacyclododecane- 1,4,7- triyl)triacetate, Isomer 2 | 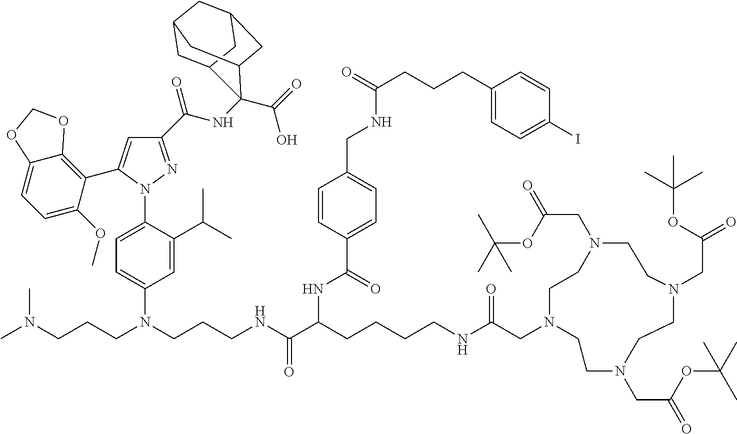 [SMILES: COc1ccc2c(c1-c1cc(C(=O)NC3(C(=O)O)C4CC5CC(C4)CC3C5)nn1-c1ccc(N(CCCNC(=O)C(CCCCNC(=O)CN3CCN(CC(=O)OC(C)(C)C)CCN(CC(=O)OC(C)(C)C)CCN(CC(=O)OC(C)(C)C)CC3)NC(=O)c3ccc(CNC(=O)CCCc4ccc(I)cc4)cc3)CCCN(C)C)cc1C(C)C)OCO2] | 930 [M/2 + H]⁺ |
| 294 | (5r,7r)-2-(1-(4- ((12,16-Dimethyl- 2,11-dioxo-1-(4,7,10- tris(2-(tert-butoxy)-2- oxoethyl)-1,4,7,10- tetraazacyclododecan- 1-yl)-6,9-dioxa- 3,12,16- triazanonadecan-19- yl)(methyl)amino)-2- isopropylphenyl)-5- (5- methoxybenzo[d][1,3] dioxol-4-yl)-1H- pyrazole-3- carboxamido)adaman- tane-2-carboxylic acid | 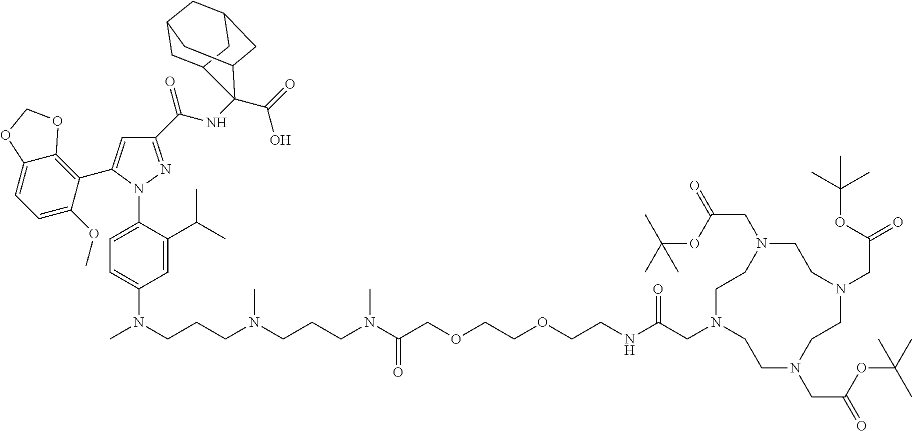 [SMILES: COc1ccc2c(c1-c1cc(C(=O)NC3(C(=O)O)C4CC5CC(C4)CC3C5)nn1-c1ccc(N(C)CCCN(C)CCCN(C)C(=O)COCCOCCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 715 [M/2 + H]⁺ |
| 368 | tri-tert-Butyl 2,2′,2″- (10-(2-((3-((1R,4R)- 5-(4-(3-(((5R,7R)-2- (tert- butoxycarbonyl)adaman- tan-2- yl)carbamoyl)-5-(5- methoxybenzo[d][1,3] dioxol-4-yl)-1H- pyrazol-1-yl)-3- isopropylphenyl)-2,5- diazabicyclo[2.2.1]hep- tan-2- yl)propyl)amino)-2- oxoethyl)-1,4,7,10- tetraazacyclododecane- 1,4,7-triyl)triacetate | 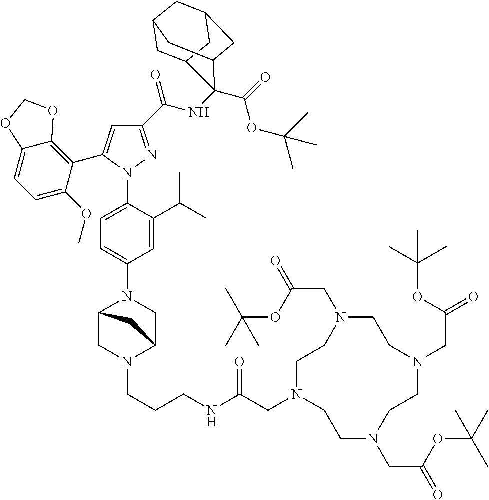 [SMILES: COc1ccc2c(c1-c1cc(C(=O)NC3(C(=O)OC(C)(C)C)C4CC5CC(C4)CC3C5)nn1-c1ccc(N3C[C@H]4C[C@@H]3CN4CCCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 1322 [M + H]⁺ |
| 369 | tri-tert-butyl 2,2′,2″- (10-(2-((3-((1S,4S)- 5-(4-(3-(((5S,7S)-2- (tert- butoxycarbonyl)adaman- tan-2- yl)carbamoyl)-5-(5- methoxybenzo[d][1,3] dioxol-4-yl)-1H- pyrazol-1-yl)-3- isopropylphenyl)-2,5- diazabicyclo[2.2.1]hep- tan-2- yl)propyl)amino)-2- oxoethyl)-1,4,7,10- tetraazacyclododecane- 1,4,7-triyl)triacetate | 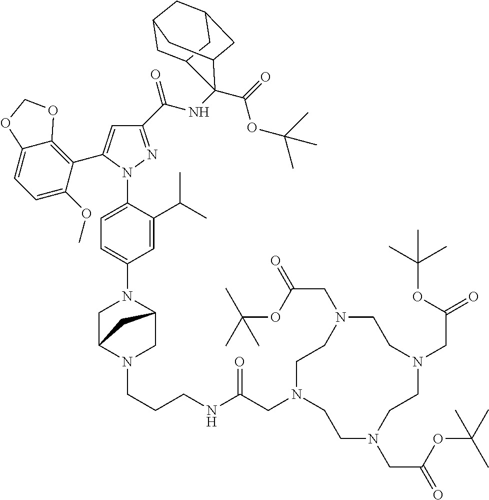 [SMILES: COc1ccc2c(c1-c1cc(C(=O)NC3(C(=O)OC(C)(C)C)C4CC5CC(C4)CC3C5)nn1-c1ccc(N3C[C@@H]4C[C@H]3CN4CCCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 1322 [M + H]⁺ |
| 370 | tri-tert-Butyl 2,2′,2″- (10-(2-((3-(7-(4-(3- (((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)-2,7- diazaspiro[3.5]nonan- 2-yl)propyl)amino)- 2-oxoethyl)-1,4,7,10- tetraazacyclododecane- 1,4,7-triyl)triacetate | 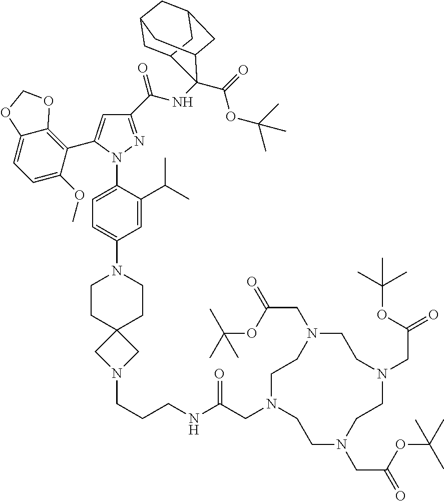 [SMILES: COc1ccc2c(c1-c1cc(C(=O)NC3(C(=O)OC(C)(C)C)C4CC5CC(C4)CC3C5)nn1-c1ccc(N3CCC4(CC3)CN(CCCNC(=O)CN3CCN(CC(=O)OC(C)(C)C)CCN(CC(=O)OC(C)(C)C)CCN(CC(=O)OC(C)(C)C)CC3)C4)cc1C(C)C)OCO2] | 1350 [M + H]⁺ |
| 371 | (5r,7r)-2-(1-(2- Isopropyl-4-(7-(3-(2- (4,7,10-tris(2-(tert- butoxy)-2-oxoethyl)- 1,4,7,10- tetraazacyclododecan- 1- yl)acetamido)propyl)- 2,7- diazaspiro[3.5]nonan- 2-yl)phenyl)-5-(5- methoxybenzo[d][1,3] dioxol-4-yl)-1H- pyrazole-3- carboxamido)adaman- tane-2-carboxylic acid | 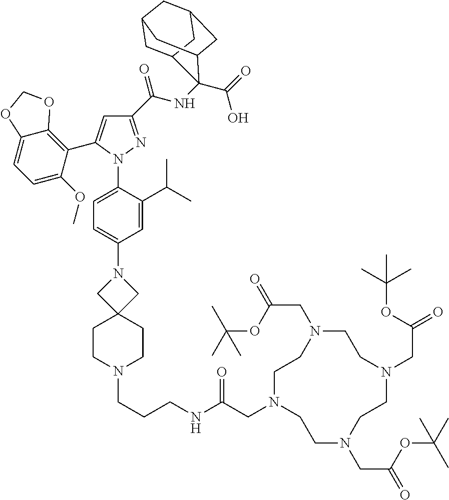 [SMILES: COc1ccc2c(c1-c1cc(C(=O)NC3(C(=O)O)C4CC5CC(C4)CC3C5)nn1-c1ccc(N3CC4(CCN(CCCNC(=O)CN5CCN(CC(=O)OC(C)(C)C)CCN(CC(=O)OC(C)(C)C)CCN(CC(=O)OC(C)(C)C)CC5)CC4)C3)cc1C(C)C)OCO2] | 1294 [M + H]⁺ |
| 372 | tri-tert-Butyl 2,2′,2″- (10-(2-((3-((3-(4-(4- (3-(((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)piper- azin-1- yl)propyl)(methyl)ami- no)propyl)amino)-2- oxoethyl)-1,4,7,10- tetraazacyclododecane- 1,4,7-triyl)triacetate | 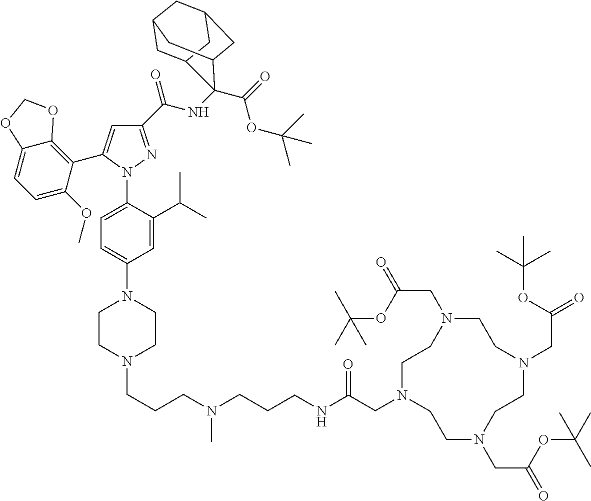 [SMILES: COc1ccc2c(c1-c1cc(C(=O)NC3(C(=O)OC(C)(C)C)C4CC5CC(C4)CC3C5)nn1-c1ccc(N3CCN(CCCN(C)CCCNC(=O)CN4CCN(CC(=O)OC(C)(C)C)CCN(CC(=O)OC(C)(C)C)CCN(CC(=O)OC(C)(C)C)CC4)CC3)cc1C(C)C)OCO2] | 1382 [M + H]⁺ |
| 373 | tri-tert-Butyl 2,2′,2″- (4-(2-((3-((3-((3-(4- (3-(((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)quino- lin-7- yl)oxy)propyl)(methyl) amino)propyl)amino)- 2-oxoethyl)-1,4,7- triazacyclododecane- 1,7,10-triyl)triacetate | 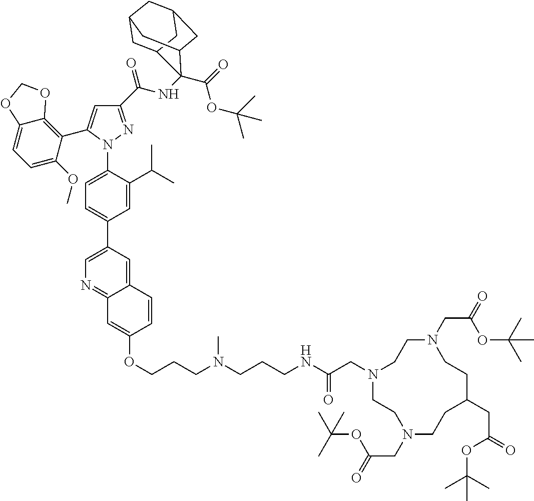 [SMILES: COc1ccc2c(c1-c1cc(C(=O)NC3(C(=O)OC(C)(C)C)C4CC5CC(C4)CC3C5)nn1-c1ccc(-c3cnc4cc(OCCCN(C)CCCNC(=O)CN5CCN(CC(=O)OC(C)(C)C)CCC(CC(=O)OC(C)(C)C)CCN(CC(=O)OC(C)(C)C)CC5)ccc4c3)cc1C(C)C)OCO2] | 1440 [M + H]⁺ |
| 374 | tri-tert-butyl 2,2′,2″- (10-(2-((3-(9-(4-(3- (((5r,7r)-2-(tert- Butoxycarbonyl)ada- mantan-2- yl)carbamoyl)-5-(5- methoxybenzo[d][1,3] dioxol-4-yl)-1H- pyrazol-1-yl)-3- isopropylphenyl)-3,9- diazaspiro[5.5]undecan- 3- yl)propyl)amino)-2- oxoethyl)-1,4,7,10- tetraazacyclododecane- 1,4,7-triyl)triacetate | 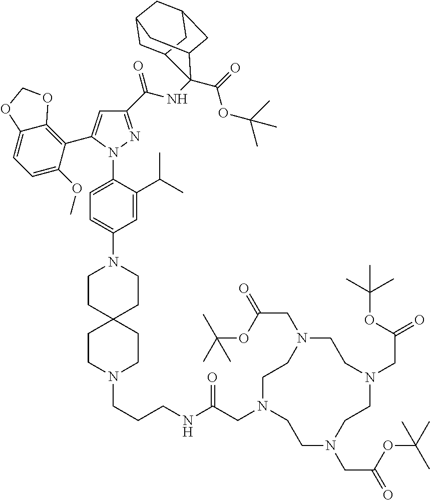 [SMILES: COc1ccc2c(c1-c1cc(C(=O)NC3(C(=O)OC(C)(C)C)C4CC5CC(C4)CC3C5)nn1-c1ccc(N3CCC4(CCN(CCCNC(=O)CN5CCN(CC(=O)OC(C)(C)C)CCN(CC(=O)OC(C)(C)C)CCN(CC(=O)OC(C)(C)C)CC5)CC4)CC3)cc1C(C)C)OCO2] | 1322 [M + H]⁺ |
| 375 | (5r,7r)-2-(1-(2- Isopropyl-4-(4-((4-(2- (4,7,10-tris(2-(tert- butoxy)-2-oxoethyl)- 1,4,7,10- tetraazacyclododecan- 1- yl)acetyl)piperazin-1- yl)methyl)piperidine- 1- carboxamido)phenyl)- 5-(5- methoxybenzo[d][1,3] dioxol-4-yl)-1H- pyrazole-3- carboxamido)adaman- tane-2-carboxylic acid | 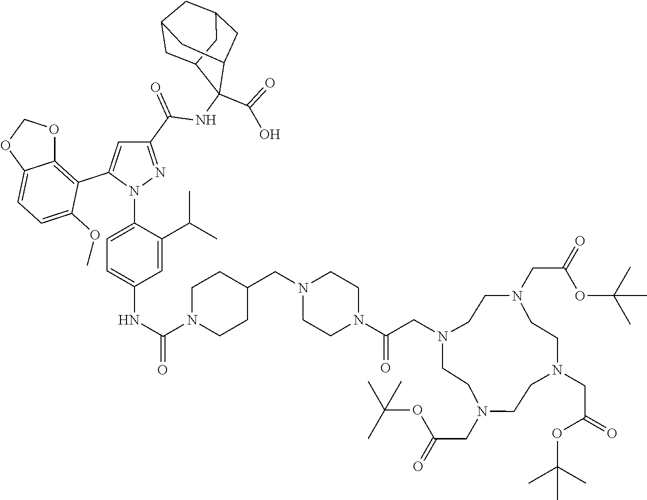 [SMILES: COc1ccc2c(c1-c1cc(C(=O)NC3(C(=O)O)C4CC5CC(C4)CC3C5)nn1-c1ccc(NC(=O)N3CCC(CN4CCN(C(=O)CN5CCN(CC(=O)OC(C)(C)C)CCN(CC(=O)OC(C)(C)C)CCN(CC(=O)OC(C)(C)C)CC5)CC4)CC3)cc1C(C)C)OCO2] | 1337 [M + H]⁺ |
| 376 | tri-tert-Butyl 2,2′,2″- (10-(2-((3-(4-(3-((4- (3-(((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)amino) propyl)piperazin-1- yl)propyl)amino)-2- oxoethyl)-1,4,7,10- tetraazacyclododecane- 1,4,7-triyl)triacetate | 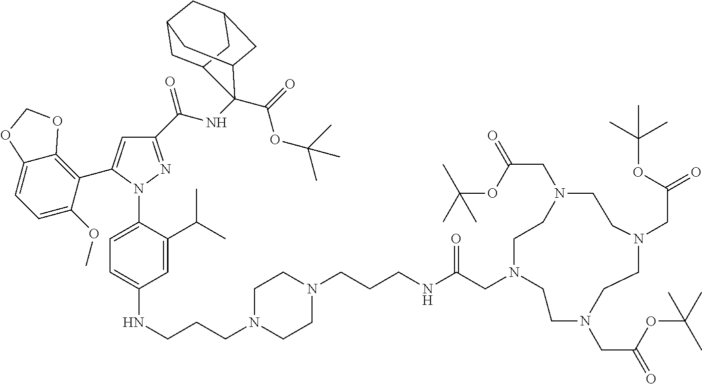 [SMILES: COc1ccc2c(c1-c1cc(C(=O)NC3(C(=O)OC(C)(C)C)C4CC5CC(C4)CC3C5)nn1-c1ccc(NCCCN3CCN(CCCNC(=O)CN4CCN(CC(=O)OC(C)(C)C)CCN(CC(=O)OC(C)(C)C)CCN(CC(=O)OC(C)(C)C)CC4)CC3)cc1C(C)C)OCO2] | 1367 [M + H]⁺ |
| 377 | (5r,7r)-2-(1-(2- Isopropyl-4-(2-(4-(2- (4,7,10-tris(2-(tert- butoxy)-2-oxoethyl)- 1,4,7,10- tetraazacyclododecan- 1- yl)acetyl)piperazin-1- yl)ethoxy)phenyl)-5- (5- methoxybenzo[d][1,3] dioxol-4-yl)-1H- pyrazole-3- carboxamido)adaman- tane-2-carboxylic acid | 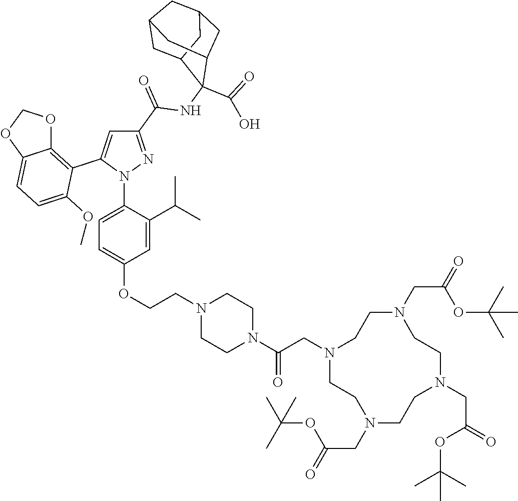 [SMILES: COc1ccc2c(c1-c1cc(C(=O)NC3(C(=O)O)C4CC5CC(C4)CC3C5)nn1-c1ccc(OCCN3CCN(C(=O)CN4CCN(CC(=O)OC(C)(C)C)CCN(CC(=O)OC(C)(C)C)CCN(CC(=O)OC(C)(C)C)CC4)CC3)cc1C(C)C)OCO2] | 1240 [M + H]⁺ |
| 378 | tri-tert-Butyl 2,2′,2″- (10-(2-((3-((4-((3-((4- (3-(((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenyl)amino) propyl)(methyl)ami- no)butyl)(methyl)ami- no)propyl)amino)-2- oxoethyl)-1,4,7,10- tetraazacyclododecane- 1,4,7-triyl)triacetate | 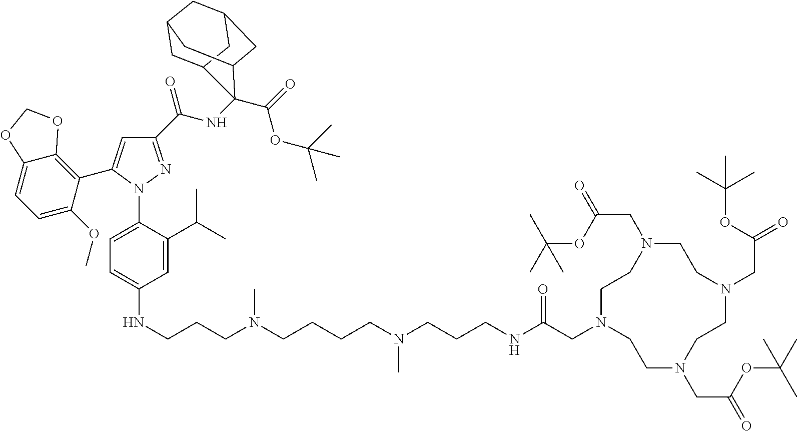 [SMILES: COc1ccc2c(c1-c1cc(C(=O)NC3(C(=O)OC(C)(C)C)C4CC5CC(C4)CC3C5)nn1-c1ccc(NCCCN(C)CCCCN(C)CCCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 1397 [M + H]⁺ |
| 379 | (5r,7r)-2-(1-(2- isopropyl-4-(3-(3-(2- (4,7,10-tris(2-(tert- butoxy)-2-oxoethyl)- 1,4,7,10- tetraazacyclododecan- 1- yl)acetamido)propoxy) propoxy)phenyl)-5- (5- methoxybenzo[d][1,3] dioxol-4-yl)-1H- pyrazole-3- carboxamido)adaman- tane-2-carboxylic acid | 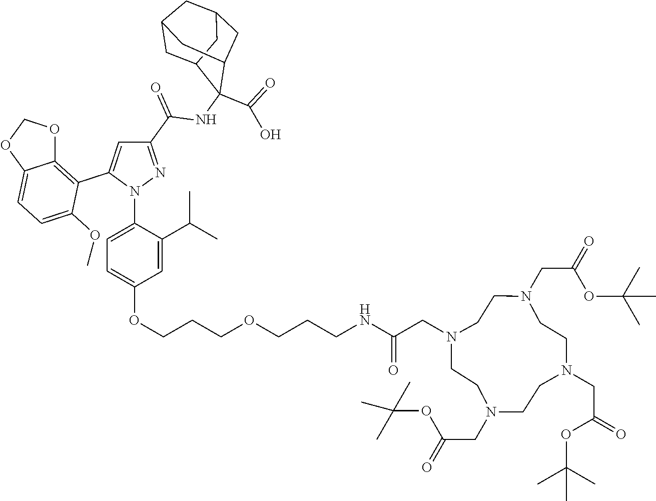 [SMILES: COc1ccc2c(c1-c1cc(C(=O)NC3(C(=O)O)C4CC5CC(C4)CC3C5)nn1-c1ccc(OCCCOCCCNC(=O)CN3CCN(CC(=O)OC(C)(C)C)CCN(CC(=O)OC(C)(C)C)CCN(CC(=O)OC(C)(C)C)CC3)cc1C(C)C)OCO2] | 1244 [M + H]⁺ |
| 380 | tri-tert-Butyl 2,2′,2″- (10-(2-(((2S)-5-(tert- butoxy)-1-(4-(2-(4- (3-(((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3- isopropylphenoxy)eth- yl)piperidin-1-yl)- 1,5-dioxopentan-2- yl)amino)-2- oxoethyl)-1,4,7,10- tetraazacyclododecane- 1,4,7-triyl)triacetate | 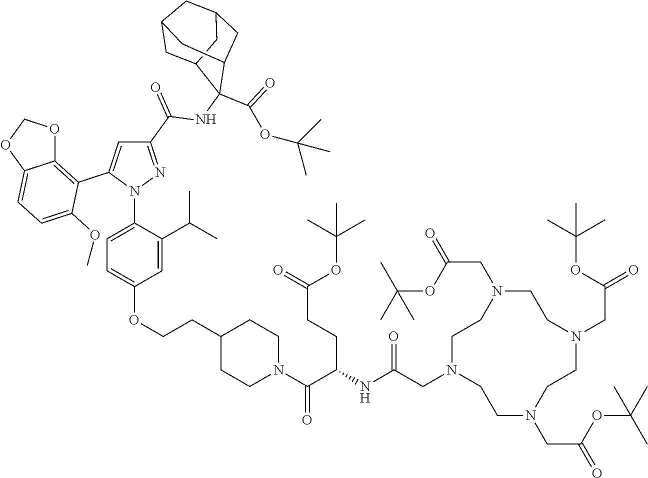 [SMILES: COc1ccc2c(c1-c1cc(C(=O)NC3(C(=O)OC(C)(C)C)C4CC5CC(C4)CC3C5)nn1-c1ccc(OCCC3CCN(C(=O)[C@H](CCC(=O)OC(C)(C)C)NC(=O)CN4CCN(CC(=O)OC(C)(C)C)CCN(CC(=O)OC(C)(C)C)CCN(CC(=O)OC(C)(C)C)CC4)CC3)cc1C(C)C)OCO2] | 1481 [M + H]⁺ |
| 381 | (5r,7r)-2-(1-(2- isopropyl-4-(3-(4-(2- (4,7,10-tris(2-(tert- butoxy)-2-oxoethyl)- 1,4,7,10- tetraazacyclododecan- 1- yl)acetyl)piperazin-1- yl)propoxy)phenyl)- 5-(5- methoxybenzo[d][1,3] dioxol-4-yl)-1H- pyrazole-3- carboxamido)adaman- tane-2-carboxylic acid | 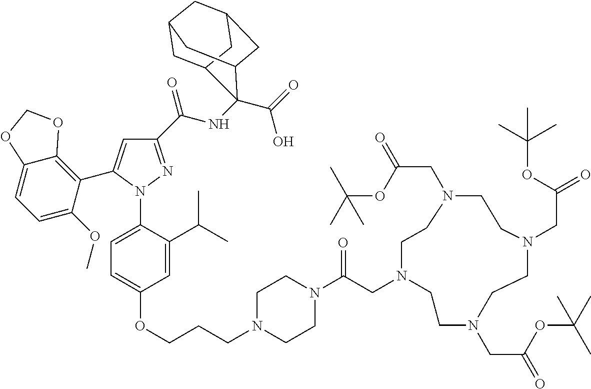 [SMILES: COc1ccc2c(c1-c1cc(C(=O)NC3(C(=O)O)C4CC5CC(C4)CC3C5)nn1-c1ccc(OCCCN3CCN(C(=O)CN4CCN(CC(=O)OC(C)(C)C)CCN(CC(=O)OC(C)(C)C)CCN(CC(=O)OC(C)(C)C)CC4)CC3)cc1C(C)C)OCO2] | 1255 [M + H]⁺ |
| 382 | (5r,7r)-2-(1-(2- Isopropyl-4-(4-(2-(2- (4,7,10-tris(2-(tert- butoxy)-2-oxoethyl)- 1,4,7,10- tetraazacyclododecan- 1- yl)acetamido)ethoxy) piperidine-1- carboxamido)phenyl)- 5-(5- methoxybenzo[d][1,3] dioxol-4-yl)-1H- pyrazole-3- carboxamido)adaman- tane-2-carboxylic acid | 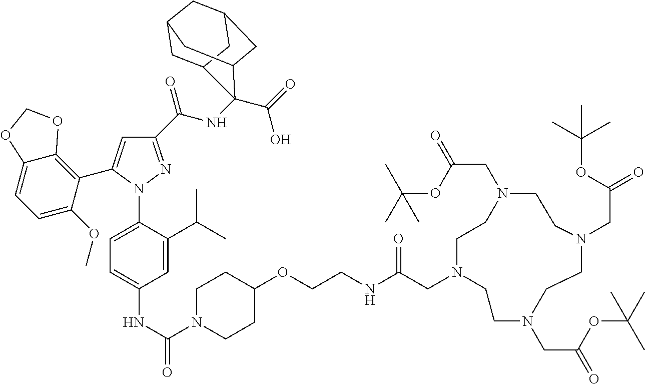 [SMILES: COc1ccc2c(c1-c1cc(C(=O)NC3(C(=O)O)C4CC5CC(C4)CC3C5)nn1-c1ccc(NC(=O)N3CCC(OCCNC(=O)CN4CCN(CC(=O)OC(C)(C)C)CCN(CC(=O)OC(C)(C)C)CCN(CC(=O)OC(C)(C)C)CC4)CC3)cc1C(C)C)OCO2] | 1298 [M + H]⁺ |
| 383 | tri-tert-Butyl 2,2′,2″- (10-(2-((3-((3-((4′-(3- (((5r,7r)-2-(tert- butoxycarbonyl)adaman- tan-2- yl)carbamoyl)-5-(5- methoxybenzo[d][1,3] dioxol-4-yl)-1H- pyrazol-1-yl)-3′- isopropyl-[1,1′- biphenyl]-4- yl)oxy)propyl)(methyl) amino)propyl)amino)- 2-oxoethyl)- 1,4,7,10- tetraazacyclododecane- 1,4,7-triyl)triacetate | 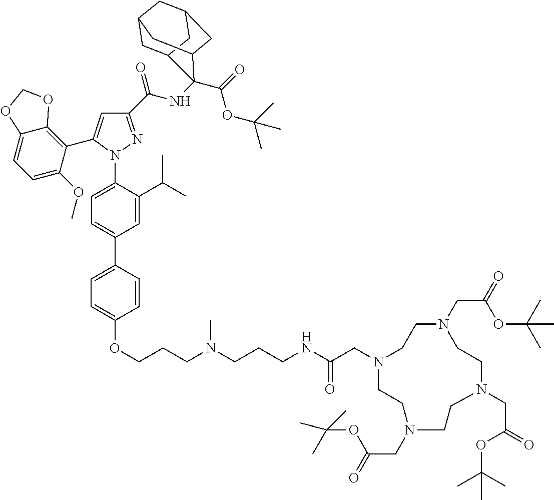 [SMILES: COc1ccc2c(c1-c1cc(C(=O)NC3(C(=O)OC(C)(C)C)C4CC5CC(C4)CC3C5)nn1-c1ccc(-c3ccc(OCCCN(C)CCCNC(=O)CN4CCN(CC(=O)OC(C)(C)C)CCN(CC(=O)OC(C)(C)C)CCN(CC(=O)OC(C)(C)C)CC4)cc3)cc1C(C)C)OCO2] | 1389 [M + H]⁺ |
| 384 | (5r,7r)-2-(1-(2- Isopropyl-4-(2-oxo-9- (3-(2-(4,7,10-tris(2- (tert-butoxy)-2- oxoethyl)-1,4,7,10- tetraazacyclododecan- 1- yl)acetamido)propyl)- 3,9- diazaspiro[5.5]undecan- 3-yl)phenyl)-5-(5- methoxybenzo[d][1,3] dioxol-4-yl)-1H- pyrazole-3- carboxamido)adaman- tane-2-carboxylic acid | 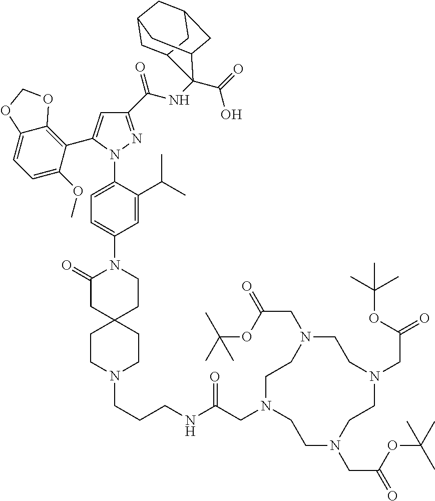 [SMILES: COc1ccc2c(c1-c1cc(C(=O)NC3(C(=O)O)C4CC5CC(C4)CC3C5)nn1-c1ccc(N3CCC4(CCN(CCCNC(=O)CN5CCN(CC(=O)OC(C)(C)C)CCN(CC(=O)OC(C)(C)C)CCN(CC(=O)OC(C)(C)C)CC5)CC4)CC3=O)cc1C(C)C)OCO2] | 1336 [M + H]⁺ |
| 385 | (5r,7r)-2-(1-(4-(3-(3- ((3-(5-(tert-Butoxy)- 5-oxo-4-(4,7,10- tris(2-(tert-butoxy)-2- oxoethyl)-1,4,7,10- tetraazacyclododecan- 1- yl)pentanamido)propyl) (methyl)amino)pro- pyl)ureido)-2- isopropylphenyl)-5- (5- methoxybenzo[d][1,3] dioxol-4-yl)-1H- pyrazole-3- carboxamido)adaman- tane-2-carboxylic acid | 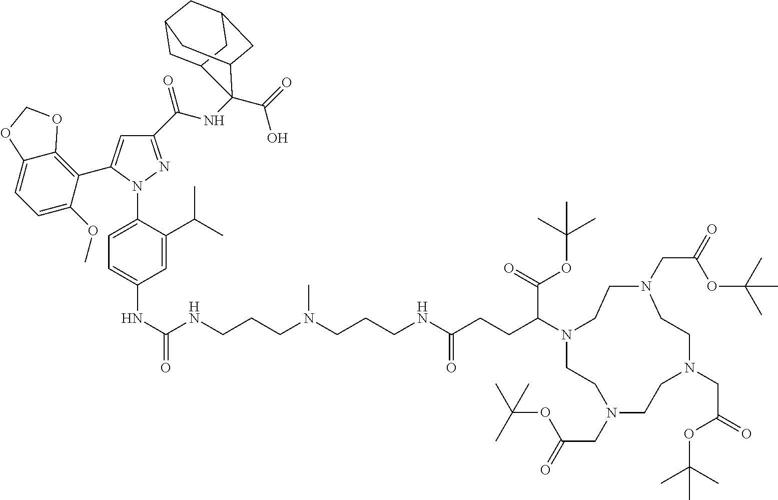 [SMILES: COc1ccc2c(c1-c1cc(C(=O)NC3(C(=O)O)C4CC5CC(C4)CC3C5)nn1-c1ccc(NC(=O)NCCCN(C)CCCNC(=O)CCC(C(=O)OC(C)(C)C)N3CCN(CC(=O)OC(C)(C)C)CCN(CC(=O)OC(C)(C)C)CCN(CC(=O)OC(C)(C)C)CC3)cc1C(C)C)OCO2] | 1427 [M + H]⁺ |
| 386 | (5r,7r)-2-(1-(2- Isopropyl-4-(5-(3- (methyl(3-(2-(4,7,10- tris(2-(tert-butoxy)-2- oxoethyl)-1,4,7,10- tetraazacyclododecan- 1- yl)acetamido)propyl) amino)propyl)-1,3,4- oxadiazol-2- yl)phenyl)-5-(5- methoxybenzo[d][1,3] dioxol-4-yl)-1H- pyrazole-3- carboxamido)adaman- tane-2-carboxylic acid | 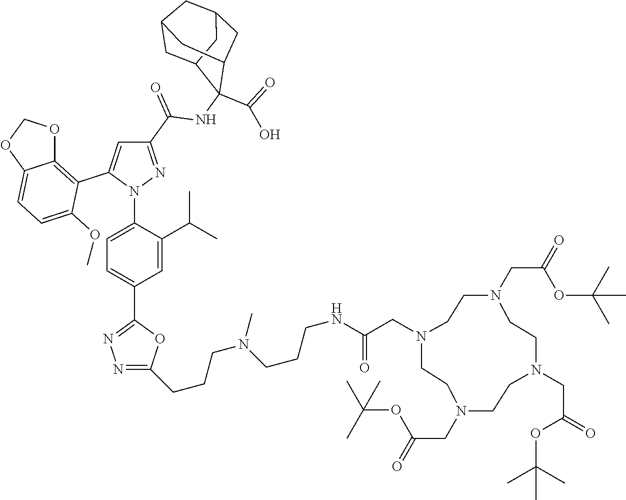 [SMILES: COc1ccc2c(c1-c1cc(C(=O)NC3(C(=O)O)C4CC5CC(C4)CC3C5)nn1-c1ccc(-c3nnc(CCCN(C)CCCNC(=O)CN4CCN(CC(=O)OC(C)(C)C)CCN(CC(=O)OC(C)(C)C)CCN(CC(=O)OC(C)(C)C)CC4)o3)cc1C(C)C)OCO2] | 655 [M/2 + H]⁺ |
| 387 | (5r,7r)-2-(1-(2- Isopropyl-4-(2-(4-(3- (2-(4,7,10-tris(2-(tert- butoxy)-2-oxoethyl)- 1,4,7,10- tetraazacyclododecan- 1- yl)acetamido)propyl) piperazin-1- yl)ethoxy)phenyl)-5- (5- methoxybenzo[d][1,3] dioxol-4-yl)-1H- pyrazole-3- carboxamido)adaman- tane-2-carboxylic acid | 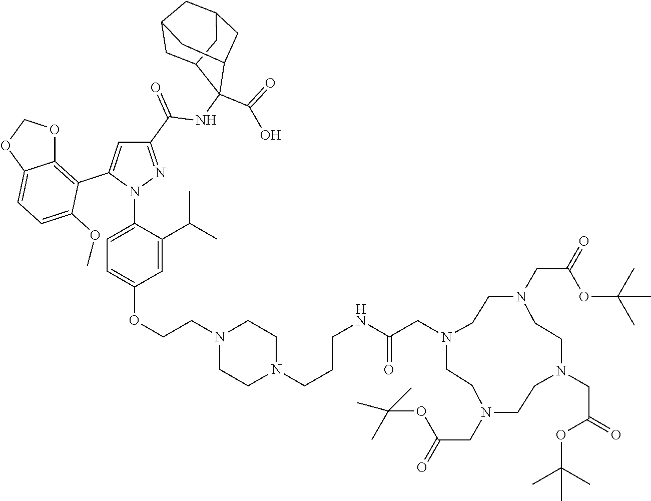 [SMILES: COc1ccc2c(c1-c1cc(C(=O)NC3(C(=O)O)C4CC5CC(C4)CC3C5)nn1-c1ccc(OCCN3CCN(CCCNC(=O)CN4CCN(CC(=O)OC(C)(C)C)CCN(CC(=O)OC(C)(C)C)CCN(CC(=O)OC(C)(C)C)CC4)CC3)cc1C(C)C)OCO2] | 1298 [M + H]⁺ |

### Preparation 295

### 1-(2-Isopropyl-4-(methyl(3-(methyl(3-(N-methyl-2-(4,7,10-tris(2-(tert-butoxy)-2-oxoethyl)-1,4,7,10-tetraazacyclododecan-1-yl)acetamido)propyl)amino)propyl)carbamoyl)phenyl)-5-(5-methoxybenzo[d][1,3]dioxol-4-yl)-1H-pyrazole-3-carboxylic acid

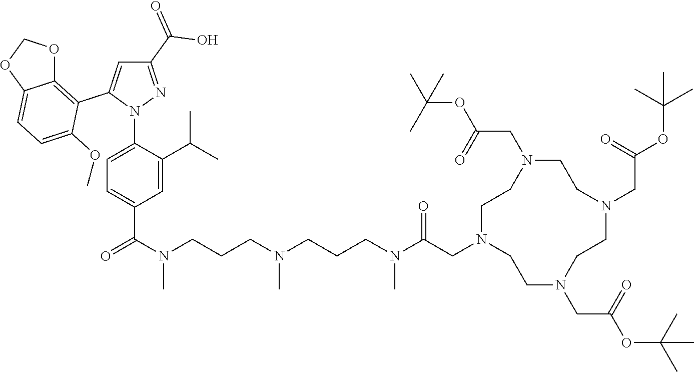 [SMILES: COc1ccc2c(c1-c1cc(C(=O)O)nn1-c1ccc(C(=O)N(C)CCCN(C)CCCN(C)C(=O)CN3CCN(CC(=O)OC(C)(C)C)CCN(CC(=O)OC(C)(C)C)CCN(CC(=O)OC(C)(C)C)CC3)cc1C(C)C)OCO2]

A soln of tri-tert-butyl 2,2′,2″-(10-(2-((3-((3-(4-(5-(benzo[1,2-d:4,5-d′]bis([1,3]dioxole)-4-yl)-3-(ethoxycarbonyl)-1H-pyrazol-1-yl)-3-isopropyl-N-methylbenzamido)propyl)(methyl)amino)propyl)(methyl)amino)-2-oxoethyl)-1,4,7,10-tetraazacyclododecane-1,4,7-triyl)triacetate (320 mg, 0.272 mmol) in THF (3 mL), H₂O (1 mL) and MeOH (1 mL) was treated with LiOH (32.6 mg, 1.36 mmol). After stirring at 40° C. for 2.5 h, the pH of the mixture was adjusted to 7 with 1N HCl. The mixture was extracted with iPrOH/CHCl₃ (3:1) (2×5 mL). The combined organic layers were washed with brine, dried over MgSO₄ and concentrated to afford crude title compound (300 mg, 96%). MS ES+ m/z 1149 [M+H]⁺.

### Preparation 296

### tri-tert-Butyl 2,2′,2″-(10-(2-((3-((3-(4-(5-(benzo[1,2-d:4,5-d′]bis([1,3]dioxole)-4-yl)-3-(((5r,7r)-2-(tert-butoxycarbonyl)adamantan-2-yl)carbamoyl)-1H-pyrazol-1-yl)-3-isopropyl-N-methylbenzamido)propyl)(methyl)amino)propyl)(methyl)amino)-2-oxoethyl)-1,4,7,10-tetraazacyclododecane-1,4,7-triyl)triacetate

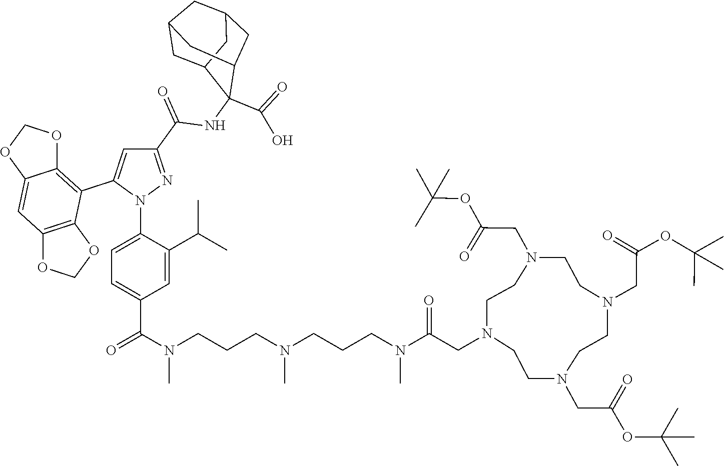 [SMILES: CC(C)c1cc(C(=O)N(C)CCCN(C)CCCN(C)C(=O)CN2CCN(CC(=O)OC(C)(C)C)CCN(CC(=O)OC(C)(C)C)CCN(CC(=O)OC(C)(C)C)CC2)ccc1-n1nc(C(=O)NC2(C(=O)O)C3CC4CC(C3)CC2C4)cc1-c1c2c(cc3c1OCO3)OCO2]

A soln of 5-(benzo[1,2-d:4,5-d′]bis([1,3]dioxole)-4-yl)-1-(2-isopropyl-4-(methyl(3-(methyl(3-(N-methyl-2-(4,7,10-tris(2-(tert-butoxy)-2-oxoethyl)-1,4,7,10-tetraazacyclododecan-1-yl)acetamido)propyl)amino)propyl)carbamoyl)phenyl)-1H-pyrazole-3-carboxylic acid (300 mg, 0.261 mmol) in DMF (3 mL) was treated with tert-butyl (5r,7r)-2-aminoadamantane-2-carboxylate (98.5 mg, 0.392 mmol), HATU (298 mg, 0.784 mmol) and DIEA (0.228 mL, 1.31 mmol). After stirring at 40° C. for 1 h, the mixture was diluted with H₂O and extracted CHCl₃/iPrOH (3:1) (3×10 mL). The combined organic layers were concentrated and purified by reversed-phase flash chromatography with the following conditions: column, C18; mobile phase, 5% to 50% DCM (0.3% Et₃N) in MeOH to provide title compound (150 mg, 42%). MS ES+ m/z 1383 [M+H]⁺.

### Preparation 297

### tri-tert-Butyl 2,2′,2″-(10-(1-((4-(3-(((5r,7r)-2-(tert-butoxycarbonyl)adamantan-2-yl)carbamoyl)-5-(5-methoxybenzo[d][1,3]dioxol-4-yl)-1H-pyrazol-1-yl)-3-isopropylphenyl)amino)-2,6,10,17,17-pentamethyl-1,11,15-trioxo-16-oxa-2,6,10-triazaoctadecan-14-yl)-1,4,7,10-tetraazacyclododecane-1,4,7-triyl)triacetate

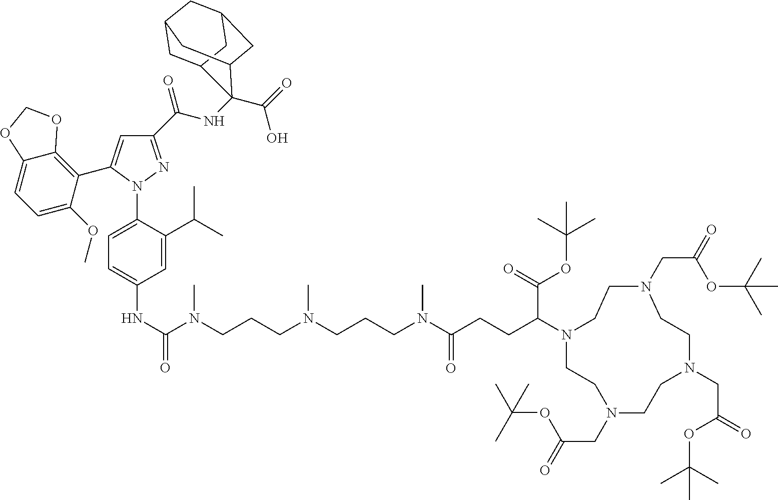 [SMILES: COc1ccc2c(c1-c1cc(C(=O)NC3(C(=O)O)C4CC5CC(C4)CC3C5)nn1-c1ccc(NC(=O)N(C)CCCN(C)CCCN(C)C(=O)CCC(C(=O)OC(C)(C)C)N3CCN(CC(=O)OC(C)(C)C)CCN(CC(=O)OC(C)(C)C)CCN(CC(=O)OC(C)(C)C)CC3)cc1C(C)C)OCO2]

A soln of tert-butyl (5r,7r)-2-(1-(2-isopropyl-4-(3-methyl-3-(3-(methyl(3-(methylamino)propyl)amino)propyl) ureido) phenyl)-5-(5-methoxybenzo[d][1,3]dioxol-4-yl)-1H-pyrazole-3-carboxamido)adamantane-2-carboxylate (54 mg, 0.065 mmol) in DMF (2 mL) and DIEA (0.11 mL, 0.65 mmol) was treated with 5-(tert-butoxy)-5-oxo-4-(4,7,10-tris(2-(tert-butoxy)-2-oxoethyl)-1,4,7,10-tetraazacyclododecan-1-yl)pentanoic acid (55 mg, 0.078 mmol) and T3P (0.12 mL, 50% wt, 0.20 mmol). After stirring at 21° C. for 4 h, the mixture was treated with sat NH₄Cl and extracted with CHCl₃/iPrOH (3:1) (4×4 mL). The combined organic layers were concentrated and purified by reversed-phase flash chromatography with the following conditions: column, C18; mobile phase, 5% to 50% ACN in H₂O (0.1% FA) to provide title compound (15 mg, 15%). MS ES+ m/z 1512 [M+H]⁺.

### Preparation 77

### (5r,7r)-2-(1-(2-Isopropyl-4-(3-(methyl(2-(4-(N-methyl-2-(4,7,10-tris(2-(tert-butoxy)-2-oxoethyl)-1,4,7,10-tetraazacyclododecan-1-yl)acetamido)butanamido)ethyl)amino)propoxy)phenyl)-5-(5-methoxybenzo[d][1,3]dioxol-4-yl)-1H-pyrazole-3-carboxamido)adamantane-2-carboxylic acid

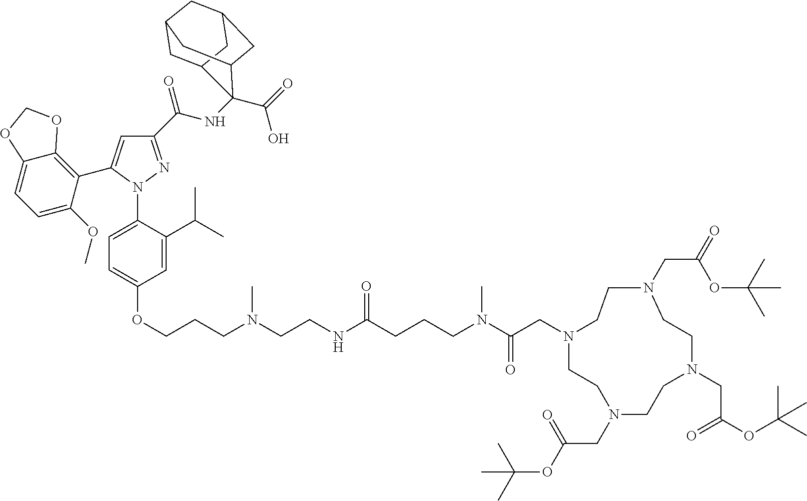 [SMILES: COc1ccc2c(c1-c1cc(C(=O)NC3(C(=O)O)C4CC5CC(C4)CC3C5)nn1-c1ccc(OCCCN(C)CCNC(=O)CCCN(C)C(=O)CN3CCN(CC(=O)OC(C)(C)C)CCN(CC(=O)OC(C)(C)C)CCN(CC(=O)OC(C)(C)C)CC3)cc1C(C)C)OCO2]

A soln of 4-(N-methyl-2-(4,7,10-tris(2-(tert-butoxy)-2-oxoethyl)-1,4,7,10-tetraazacyclododecan-1-yl)acetamido)butanoic acid (68 mg, 0.10 mmol), TBTU (65 mg, 0.20 mmol), DIEA (79 mg, 0.61 mmol) in DMF (3 mL) was treated with (5r,7r)-2-(1-(4-(3-((2-aminoethyl)(methyl)amino)propoxy)-2-isopropylphenyl)-5-(5-methoxybenzo[d][1,3]dioxol-4-yl)-1H-pyrazole-3-carboxamido)adamantane-2-carboxylic acid (70 mg, 0.10 mmol) in DMF (1 mL). After stirring at RT for 3 h the mixture was purified by reversed-phase flash chromatography with the following conditions: column, C18; mobile phase, 0% to 60% ACN in H₂O (0.1% FA) to provide title compound (50 mg, 37%) as white foam. MS ES+ m/z 1342 [M+H]⁺.

The following compounds were prepared in a manner essentially analogous to the preparation method of (5r,7r)-2-(1-(2-Isopropyl-4-(3-(methyl(2-(4-(N-methyl-2-(4,7,10-tris(2-(tert-butoxy)-2-oxoethyl)-1,4,7,10-tetraazacyclododecan-1-yl)acetamido)butanamido)ethyl)amino)propoxy)phenyl)-5-(5-methoxybenzo[d][1,3]dioxol-4-yl)-1H-pyrazole-3-carboxamido)adamantane-2-carboxylic acid using the appropriate reagents, adjusting temperature, adjusting reaction time to determine completion of the reaction, and adjusting the purification system as appropriate.

TABLE 18

| Prep | | | |
|---|---|---|---|
| # | Chemical Name | Structure | MS ES+ m/z |
| 78 | tri-tert-Butyl 2,2′,2″- (10-(2-((3-((3-((4-(3- (((5r,7r)-2-(tert- butoxycarbonyl) adamantan-2-yl) carbamoyl)-5-(5- methoxybenzo[d] [1,3]dioxol-4-yl)- 1H-pyrazol-1-yl)-3- isopropylphenyl) (methyl)amino) propyl)(methyl) amino)propyl) (methyl)amino)-2- oxoethyl)-1,4,7,10- tetraazacyclo- dodecane-1,4,7- triyl)triacetate | 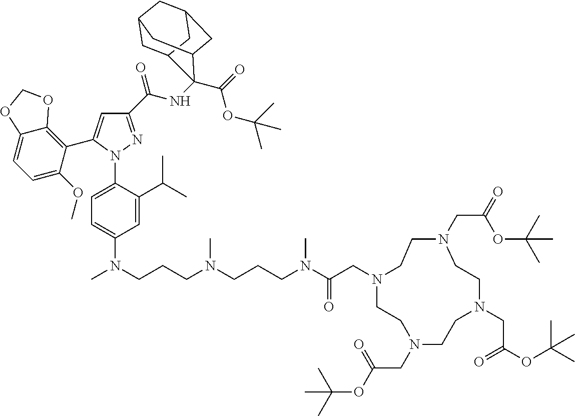 [SMILES: COc1ccc2c(c1-c1cc(C(=O)NC3(C(=O)OC(C)(C)C)C4CC5CC(C4)CC3C5)nn1-c1ccc(N(C)CCCN(C)CCCN(C)C(=O)CN3CCN(CC(=O)OC(C)(C)C)CCN(CC(=O)OC(C)(C)C)CCN(CC(=O)OC(C)(C)C)CC3)cc1C(C)C)OCO2] | 1340 [M + H]⁺ |
| 79 | tri-tert-Butyl 2,2′,2″- (10-(2-((3-((3-(3-(4- (3-(((5r,7r)-2-(tert-butoxy- carbonyl)adamantan-2- yl)carbamoyl)-5-(5- methoxybenzo[d][1,3] dioxol-4-yl)-1H-pyrazol- 1-yl)-3-isopropylphenyl)- 1-methylureido)propyl) (methyl)amino)propyl) (methyl)amino)-2- oxoethyl)-1,4,7,10- tetraazacyclododecane- 1,4,7-triyl)triacetate | 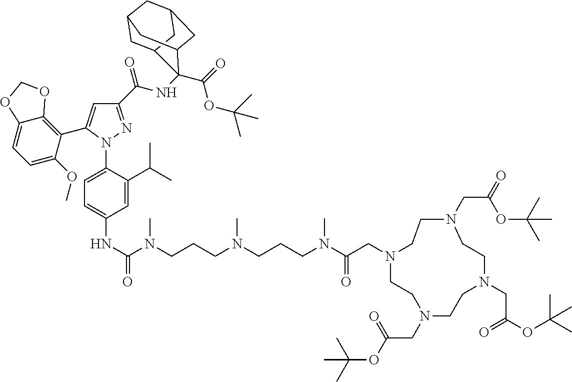 [SMILES: COc1ccc2c(c1-c1cc(C(=O)NC3(C(=O)OC(C)(C)C)C4CC5CC(C4)CC3C5)nn1-c1ccc(NC(=O)N(C)CCCN(C)CCCN(C)C(=O)CN3CCN(CC(=O)OC(C)(C)C)CCN(CC(=O)OC(C)(C)C)CCN(CC(=O)OC(C)(C)C)CC3)cc1C(C)C)OCO2] | 1383 [M + H]⁺ |

### Preparation 404

### tri-tert-Butyl 2,2′,2″-(10-(2-((3-((3-(4-(3-(((5r,7r)-2-(tert-butoxycarbonyl)adamantan-2-yl)carbamoyl)-5-(5-cyanobenzo[d][1,3]dioxol-4-yl)-1H-pyrazol-1-yl)-3-isopropylphenoxy)propyl)(methyl)amino)propyl)amino)-2-oxoethyl)-1,4,7,10-tetraazacyclododecane-1,4,7-triyl)triacetate

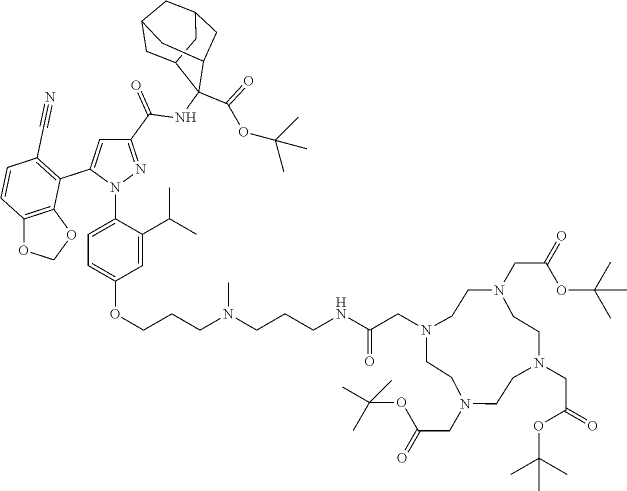 [SMILES: CC(C)c1cc(OCCCN(C)CCCNC(=O)CN2CCN(CC(=O)OC(C)(C)C)CCN(CC(=O)OC(C)(C)C)CCN(CC(=O)OC(C)(C)C)CC2)ccc1-n1nc(C(=O)NC2(C(=O)OC(C)(C)C)C3CC4CC(C3)CC2C4)cc1-c1c(C#N)ccc2c1OCO2]

A soln of PPh₃ (503 mg, 1.9 mmol) in THF (10 mL) at 0° C. under N₂, was treated with DIAD (427 mg, 2.1 mmol) and stirred for 10 min prior to treatment with tert-butyl 2-{4,7-bis[2-(tert-butoxy)-2-oxoethyl]-10-[({3-[(3-hydroxypropyl)(methyl)amino]propyl}carbamoyl)methyl]-1,4,7,10-tetraazacyclododecan-1-yl}acetate (898 mg, 1.3 mmol) and tert-butyl 2-[5-(5-cyano-2H-1,3-benzodioxol-4-yl)-1-(4-hydroxy-2-isopropylphenyl)pyrazole-3-amido]adamantane-2-carboxylate (400 mg, 0.64 mmol). The mixture was stirred ON at RT, concentrated under reduced pressure, and purified by reversed-phase flash chromatography with the following conditions: column, C18; mobile phase, 10% to 50% ACN in H₂O (0.1% TFA) to provide title compound (470 mg, 56%) as white solid. MS ES+ m/z 1308 [M+H]⁺.

### Example A

(5r,7r)-2-(1-(4-((((3-(Dimethylamino)propyl)thio) carbonyl)amino)-2-isopropylphenyl)-5-(5-methoxybenzo[d][1,3]dioxol-4-yl)-1H-pyrazole-3-carboxamido)adamantane-2-carboxylic acid

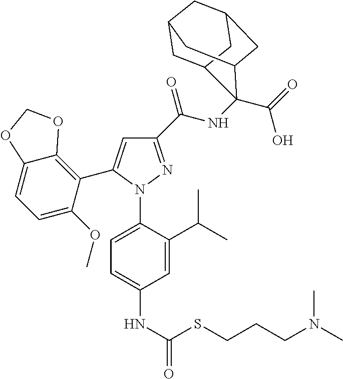 [SMILES: COc1ccc2c(c1-c1cc(C(=O)NC3(C(=O)O)C4CC5CC(C4)CC3C5)nn1-c1ccc(NC(=O)SCCCN(C)C)cc1C(C)C)OCO2]

A soln of tert-butyl (5r,7r)-2-(1-(4-((((3-(dimethylamino)propyl)thio) carbonyl)amino)-2-isopropylphenyl)-5-(5-methoxybenzo[d][1,3]dioxol-4-yl)-1H-pyrazole-3-carboxamido)adamantane-2-carboxylate (46 mg, 0.059 mmol) and HCl in 1,4-dioxane (4.0 M, 0.5 mL) in DCM (3 mL) was stirred under N₂ at 50° C. for 1 h, cooled to RT, concentrated under reduced pressure, and purified by Prep-HPLC with the following conditions: Column, Xselect CSH C18 OBD 30*150 mm, 5 μm; Mobile Phase, 16% to 26% ACN in H₂O (0.1% FA) to afford title compound (28.7 mg, 67%) as a white solid. MS ES+ m/z 718 [M+H]⁺.

### Example B

### (S)-2-((3S,5S,7S)-Adamantan-1-yl)-2-(1-(4-((3-(dimethylamino)propyl)(methyl)carbamoyl)-2-isopropylphenyl)-5-(5-methoxybenzo[d][1,3]dioxol-4-yl)-1H-pyrazole-3-carboxamido) acetic acid

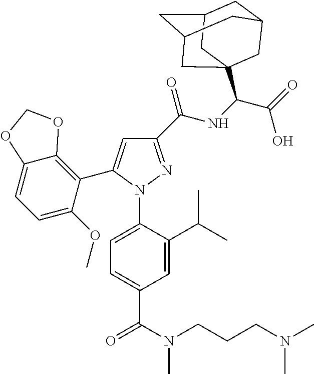 [SMILES: COc1ccc2c(c1-c1cc(C(=O)N[C@H](C(=O)O)C34CC5CC(CC(C5)C3)C4)nn1-c1ccc(C(=O)N(C)CCCN(C)C)cc1C(C)C)OCO2]

A soln of 1-(4-((3-(dimethylamino)propyl)(methyl)carbamoyl)-2-isopropylphenyl)-5-(5-methoxybenzo[d][1,3]dioxol-4-yl)-1H-pyrazole-3-carboxylic acid (30 mg, 57 μmol), HATU (44 mg, 0.11 mmol) in DMF (1.0 mL) and DIEA (0.08 mL, 0.46 mmol) was stirred for 30 min at RT. The soln was treated with (S)-2-(Adamantan-1-yl)-2-aminoacetic acid hydrochloride (28 mg, 0.11 mmol) and stirred at 22° C. for 2 h. The mixture was filtered and the filtrate purified by Prep-HPLC with the following conditions: Column, CSH C18 OBD 2.1*50 mm, 1.7 μm; Mobile Phase, 5% to 95% ACN in H₂O (10 mM NH₄OAc) to afford title compound (7 mg, 20%) as a white solid. MS ES+ m/z 714 [M+H]⁺.

### Example C

### (5r,7r)-2-(1-(2-Isopropyl-4-(2-(1-methylpyrrolidin-3-yl)acetamido) phenyl)-5-(5-methoxybenzo[d][1,3]dioxol-4-yl)-1H-pyrazole-3-carboxamido)adamantane-2-carboxylic acid

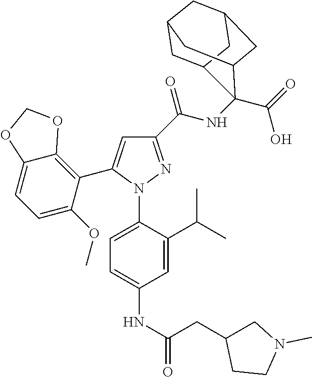 [SMILES: COc1ccc2c(c1-c1cc(C(=O)NC3(C(=O)O)C4CC5CC(C4)CC3C5)nn1-c1ccc(NC(=O)CC3CCN(C)C3)cc1C(C)C)OCO2]

A soln of tert-butyl (5r,7r)-2-(1-(2-isopropyl-4-(2-(1-methylpyrrolidin-3-yl)acetamido) phenyl)-5-(5-methoxybenzo[d][1,3]dioxol-4-yl)-1H-pyrazole-3-carboxamido)adamantane-2-carboxylate (36 mg, 48 mmol), DCM (2 mL) and TFA (1 mL, 0.01 mol) was stirred for 2 h before being concentrated and purified by Prep-HPLC with the following conditions: Column, Xselect CSH C18 OBD 30*250 mm, 5 μm; Mobile Phase, 5 to 95% ACN in H₂O (10 mM NH₄OAc) to afford title compound (6.6 mg, 20%) as a white solid. MS ES+ m/z 699 [M+H]⁺.

The following compounds were prepared in a manner essentially analogous to the preparation method of (5r,7r)-2-(1-(2-Isopropyl-4-(2-(1-methylpyrrolidin-3-yl)acetamido) phenyl)-5-(5-methoxybenzo[d][1,3]dioxol-4-yl)-1H-pyrazole-3-carboxamido)adamantane-2-carboxylic acid using the appropriate reagents, adjusting temperature, adjusting reaction time to determine completion of the reaction, and adjusting the purification system as appropriate.

TABLE 19

| Ex # | Chemical Name | Structure | MS ES+ m/z |
|---|---|---|---|
| D | (5r,7r)-2-(1-(4-((5- (Dimethylamino)pentyl) amino)-2- isopropylphenyl)-5-(5- methoxybenzo[d][1,3] dioxol-4-yl)-1H- pyrazole-3- carboxamido)adamantane- 2-carboxylic acid | 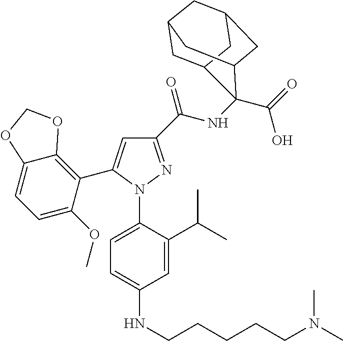 [SMILES: COc1ccc2c(c1-c1cc(C(=O)NC3(C(=O)O)C4CC5CC(C4)CC3C5)nn1-c1ccc(NCCCCCN(C)C)cc1C(C)C)OCO2] | 685 [M + H]⁺ |
| E | (5r,7r)-2-(1-(2- Isopropyl-4-(2-(1- methylpiperidin-4- yl)acetamido)phenyl)- 5-(5- methoxybenzo[d][1,3] dioxol-4-yl)-1H- pyrazole-3- carboxamido)adamantane- 2-carboxylic acid | 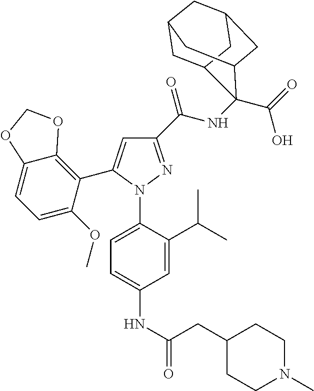 [SMILES: COc1ccc2c(c1-c1cc(C(=O)NC3(C(=O)O)C4CC5CC(C4)CC3C5)nn1-c1ccc(NC(=O)CC3CCN(C)CC3)cc1C(C)C)OCO2] | 713 [M + H]⁺ |
| F | (5r,7r)-2-(1-(4-(4- (Dimethylamino) butanamido)-2- isopropylphenyl)-5-(5- methoxybenzo[d][1,3] dioxol-4-yl)-1H- pyrazole-3- carboxamido)adamantane- 2-carboxylic acid | 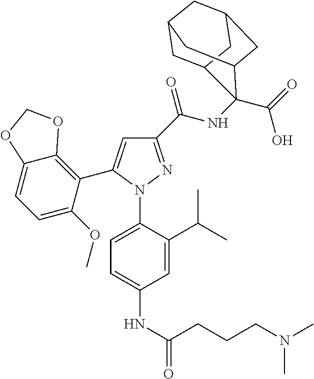 [SMILES: COc1ccc2c(c1-c1cc(C(=O)NC3(C(=O)O)C4CC5CC(C4)CC3C5)nn1-c1ccc(NC(=O)CCCN(C)C)cc1C(C)C)OCO2] | 687 [M + H]⁺ |
| G | (5r,7r)-2-(1-(4-((3- (Dimethylamino)propyl) amino)-2- isopropylphenyl)-5-(5- methoxybenzo[d][1,3] dioxol-4-yl)-1H- pyrazole-3- carboxamido)adamantane- 2-carboxylic acid | 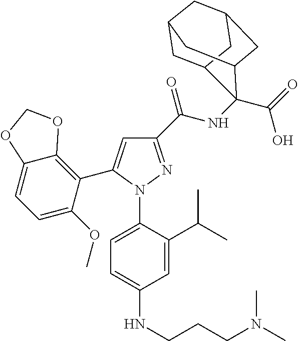 [SMILES: COc1ccc2c(c1-c1cc(C(=O)NC3(C(=O)O)C4CC5CC(C4)CC3C5)nn1-c1ccc(NCCCN(C)C)cc1C(C)C)OCO2] | 659 [M + H]⁺ |
| H | (5r,7r)-2-(1-(2-(tert- Butyl)-4-((3- (dimethylamino)propyl) amino)phenyl)-5-(5- methoxybenzo[d][1,3] dioxol-4-yl)-1H- pyrazole-3- carboxamido)adamantane- 2-carboxylic acid | 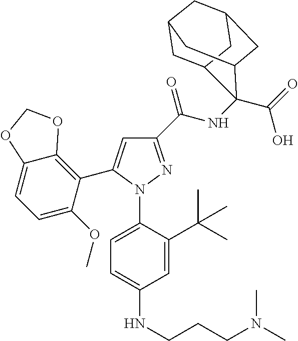 [SMILES: COc1ccc2c(c1-c1cc(C(=O)NC3(C(=O)O)C4CC5CC(C4)CC3C5)nn1-c1ccc(NCCCN(C)C)cc1C(C)(C)C)OCO2] | 672 [M + H]⁺ |
| I | (5r,7r)-2-(1-(4-((3- (Dimethylamino)propyl) (methyl)carbamoyl)-2- isopropylphenyl)-5- (5- methoxybenzo[d][1,3] dioxol-4-yl)-1H- pyrazole-3- carboxamido)adamantane- 2-carboxylic acid | 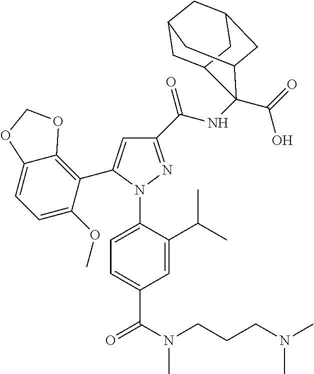 [SMILES: COc1ccc2c(c1-c1cc(C(=O)NC3(C(=O)O)C4CC5CC(C4)CC3C5)nn1-c1ccc(C(=O)N(C)CCCN(C)C)cc1C(C)C)OCO2] | 700 [M + H]⁺ |
| J | (5r,7r)-2-(1-(3′-((3- (Dimethylamino)propyl) (methyl)carbamoyl)-3- isopropyl-[1,1′- biphenyl]-4-yl)-5-(5- methoxybenzo[d][1,3] dioxol-4-yl)-1H- pyrazole-3- carboxamido)adamantane- 2-carboxylic acid | 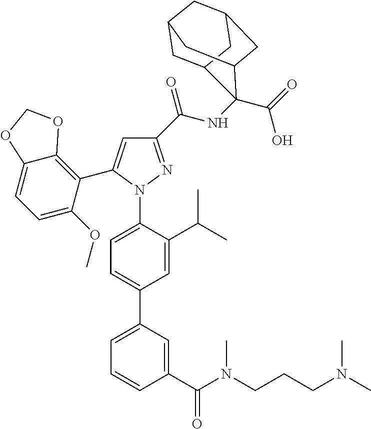 [SMILES: COc1ccc2c(c1-c1cc(C(=O)NC3(C(=O)O)C4CC5CC(C4)CC3C5)nn1-c1ccc(-c3cccc(C(=O)N(C)CCCN(C)C)c3)cc1C(C)C)OCO2] | 774 [M − H]⁻ |
| K | (5r,7r)-2-(1-(4-(1-(3- (Dimethylamino)propyl)- 1H-1,2,3-triazol-4-yl)-2- isopropylphenyl)-5-(5- methoxybenzo[d][1,3] dioxol-4-yl)-1H- pyrazole-3- carboxamido)adamantane- 2-carboxylic acid | 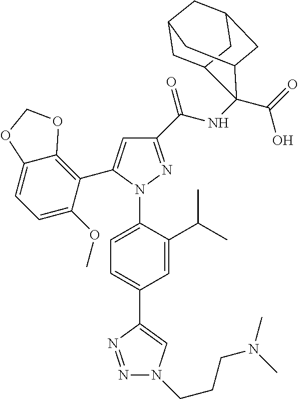 [SMILES: COc1ccc2c(c1-c1cc(C(=O)NC3(C(=O)O)C4CC5CC(C4)CC3C5)nn1-c1ccc(-c3cn(CCCN(C)C)nn3)cc1C(C)C)OCO2] | 711 [M + H]⁺ |
| L | (5r,7r)-2-(1-(4′-((3- (Dimethylamino)propyl) (methyl)carbamoyl)-3- isopropyl-[1,1′- biphenyl]-4-yl)-5-(5- methoxybenzo[d][1,3] dioxol-4-yl)-1H- pyrazole-3- carboxamido)adamantane- 2-carboxylic acid | 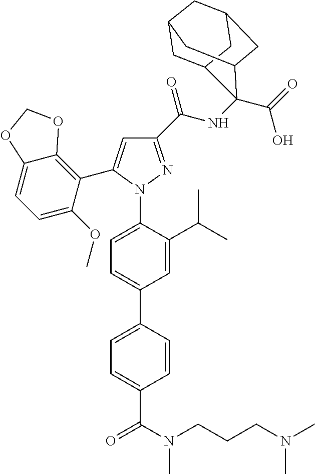 [SMILES: COc1ccc2c(c1-c1cc(C(=O)NC3(C(=O)O)C4CC5CC(C4)CC3C5)nn1-c1ccc(-c3ccc(C(=O)N(C)CCCN(C)C)cc3)cc1C(C)C)OCO2] | 776 [M + H]⁺ |
| M | (5r,7r)-2-(1-(4-(3- (Dimethylamino) propoxy)-2- isopropylphenyl)-5-(5- methoxybenzo[d][1,3] dioxol-4-yl)-1H- pyrazole-3- carboxamido)adamantane- 2-carboxylic acid | 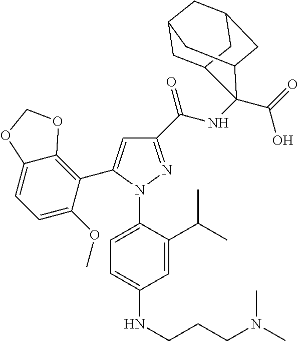 [SMILES: COc1ccc2c(c1-c1cc(C(=O)NC3(C(=O)O)C4CC5CC(C4)CC3C5)nn1-c1ccc(NCCCN(C)C)cc1C(C)C)OCO2] | 659 [M + H]⁺ |
| N | (5r,7r)-2-(1-(4′-(3- (Dimethylamino)propoxy)- 3-isopropyl-[1,1′- biphenyl]-4-yl)-5-(5- methoxybenzo[d][1,3] dioxol-4-yl)-1H- pyrazole-3- carboxamido)adamantane- 2-carboxylic acid | 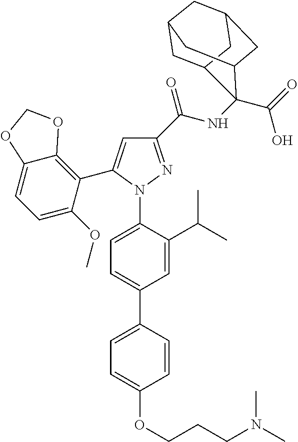 [SMILES: COc1ccc2c(c1-c1cc(C(=O)NC3(C(=O)O)C4CC5CC(C4)CC3C5)nn1-c1ccc(-c3ccc(OCCCN(C)C)cc3)cc1C(C)C)OCO2] | 736 [M + H]⁺ |
| O | (5r,7r)-2-(1-(4′-(4- (Dimethylamino) butanamido)-3- isopropyl-[1,1′-biphenyl]- 4-yl)-5-(5- methoxybenzo[d][1,3] dioxol-4-yl)-1H- pyrazole-3- carboxamido)adamantane- 2-carboxylic acid | 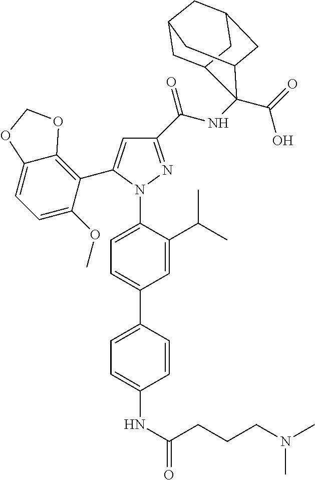 [SMILES: COc1ccc2c(c1-c1cc(C(=O)NC3(C(=O)O)C4CC5CC(C4)CC3C5)nn1-c1ccc(-c3ccc(NC(=O)CCCN(C)C)cc3)cc1C(C)C)OCO2] | 760 [M − H]⁻ |
| P | (5r,7r)-2-(1-(3′-(4- (Dimethylamino) butanamido)-3-isopropyl- [1,1′-biphenyl]-4-yl)-5-(5- methoxybenzo[d][1,3] dioxol-4-yl)-1H- pyrazole-3- carboxamido)adamantane- 2-carboxylic acid | 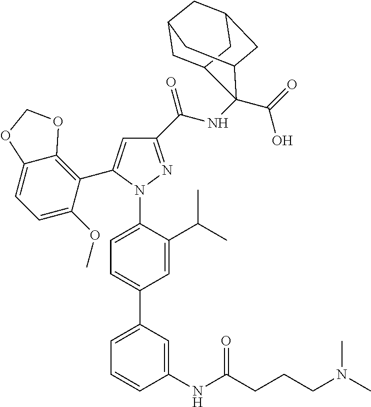 [SMILES: COc1ccc2c(c1-c1cc(C(=O)NC3(C(=O)O)C4CC5CC(C4)CC3C5)nn1-c1ccc(-c3cccc(NC(=O)CCCN(C)C)c3)cc1C(C)C)OCO2] | 760 [M − H]⁻ |
| Q | (5r,7r)-2-(1-(4′-(((3- (Dimethylamino) propoxy)carbonyl)(methyl) amino)-3-isopropyl- [1,1′-biphenyl]-4-yl)- 5-(5- methoxybenzo[d][1,3] dioxol-4-yl)-1H- pyrazole-3- carboxamido)adamantane- 2-carboxylic acid | 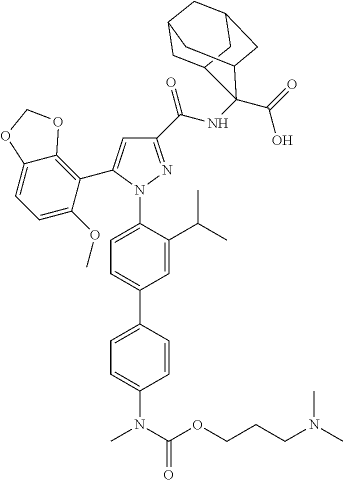 [SMILES: COc1ccc2c(c1-c1cc(C(=O)NC3(C(=O)O)C4CC5CC(C4)CC3C5)nn1-c1ccc(-c3ccc(N(C)C(=O)OCCCN(C)C)cc3)cc1C(C)C)OCO2] | 790 [M − H]⁻ |
| R | (5r,7r)-2-(1-(4′-(4- (Dimethylamino)-N- methylbutanamido)-3- isopropyl-[1,1- biphenyl]-4-yl)-5-(5- methoxybenzo[d][1,3] dioxol-4-yl)-1H- pyrazole-3- carboxamido)adamantane- 2-carboxylic acid | 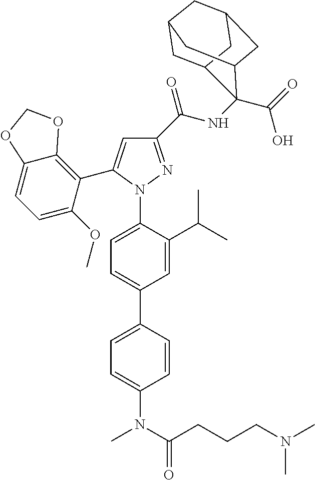 | 776 [M + H]⁺ |
| S | (5r,7r)-2-(1-(4′-(3-(3- (Dimethylamino)propyl)- 1,3-dimethylureido)-3- isopropyl-[1,1′- biphenyl]-4-yl)-5-(5- methoxybenzo[d][1,3] dioxol-4-yl)-1H- pyrazole-3- carboxamido)adamantane- 2-carboxylic acid | 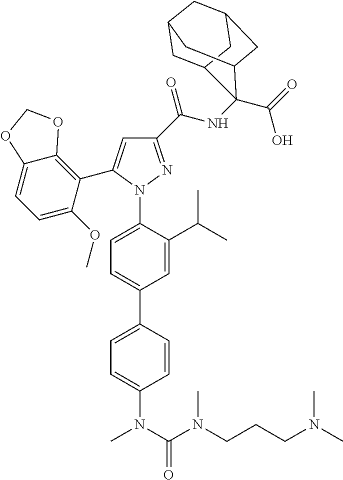 [SMILES: COc1ccc2c(c1-c1cc(C(=O)NC3(C(=O)O)C4CC5CC(C4)CC3C5)nn1-c1ccc(-c3ccc(N(C)C(=O)N(C)CCCN(C)C)cc3)cc1C(C)C)OCO2] | 806 [M + H]⁺ |
| T | (5r,7r)-2-(1-(4-((3- Azaspiro[5.5]undecan- 9-yl)amino)-2- isopropylphenyl)-5-(5- methoxybenzo[d][1,3] dioxol-4-yl)-1H- pyrazole-3- carboxamido)adamantane- 2-carboxylic acid | 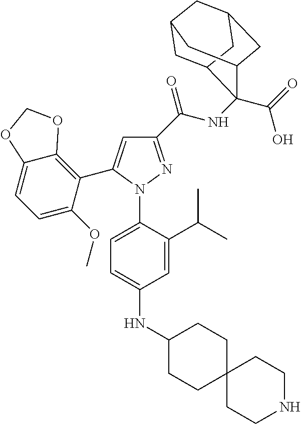 [SMILES: COc1ccc2c(c1-c1cc(C(=O)NC3(C(=O)O)C4CC5CC(C4)CC3C5)nn1-c1ccc(NC3CCC4(CCNCC4)CC3)cc1C(C)C)OCO2] | 725 [M + H]⁺ |
| U | (5r,7r)-2-(1-(2- Isopropyl-4-(1- methylpiperidine-4- carboxamido)phenyl)- 5-(5- methoxybenzo[d][1,3] dioxol-4-yl)-1H- pyrazole-3- carboxamido)adamantane- 2-carboxylic acid | 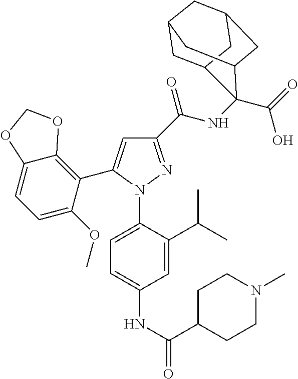 [SMILES: COc1ccc2c(c1-c1cc(C(=O)NC3(C(=O)O)C4CC5CC(C4)CC3C5)nn1-c1ccc(NC(=O)C3CCN(C)CC3)cc1C(C)C)OCO2] | 698 [M + H]⁺ |
| V | (5r,7r)-2-(1-(2- Isopropyl-4-(3-(1- methylpiperidin-4- yl)propanamido)phenyl)- 5-(5- methoxybenzo[d][1,3] dioxol-4-yl)-1H- pyrazole-3- carboxamido)adamantane- 2-carboxylic acid | 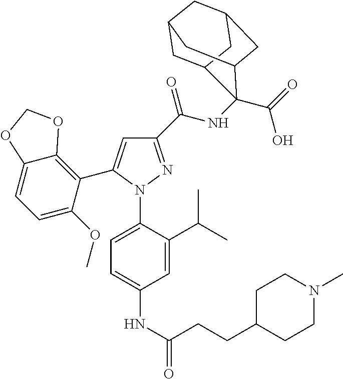 [SMILES: COc1ccc2c(c1-c1cc(C(=O)NC3(C(=O)O)C4CC5CC(C4)CC3C5)nn1-c1ccc(NC(=O)CCC3CCN(C)CC3)cc1C(C)C)OCO2] | 727 [M + H]⁺ |
| W | (5r,7r)-2-(1-(2- Isopropyl-4-(2- methyl-1-oxo-1,2,3,4- tetrahydroisoquinolin- 6-yl)phenyl)-5-(5- methoxybenzo[d][1,3] dioxol-4-yl)-1H- pyrazole-3- carboxamido)adamantane- 2-carboxylic acid | 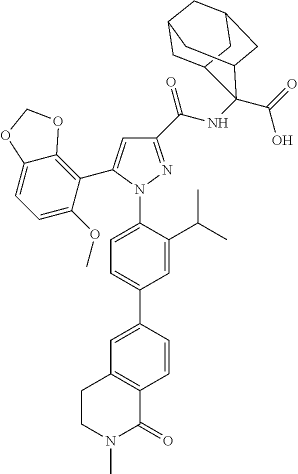 [SMILES: COc1ccc2c(c1-c1cc(C(=O)NC3(C(=O)O)C4CC5CC(C4)CC3C5)nn1-c1ccc(-c3ccc4c(c3)CCN(C)C4=O)cc1C(C)C)OCO2] | 717 [M + H]⁺ |
| X | (5r,7r)-2-(1-(2- Isopropyl-4-(2- methyl-1- oxoisoindolin-5- yl)phenyl)-5-(5- methoxybenzo[d][1,3] dioxol-4-yl)-1H- pyrazole-3- carboxamido)adamantane- 2-carboxylic acid | 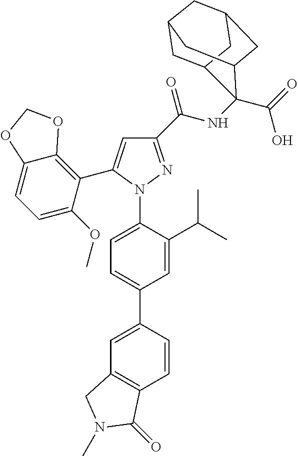 [SMILES: COc1ccc2c(c1-c1cc(C(=O)NC3(C(=O)O)C4CC5CC(C4)CC3C5)nn1-c1ccc(-c3ccc4c(c3)CN(C)C4=O)cc1C(C)C)OCO2] | 703 [M + H]⁺ |
| Y | (5r,7r)-2-(1-(2- Isopropyl-4-(9- methyl-3,9- diazaspiro[5.5]undecan- 3-yl)phenyl)-5-(5- methoxybenzo[d][1,3] dioxol-4-yl)-1H- pyrazole-3- carboxamido)adamantane- 2-carboxylic acid | 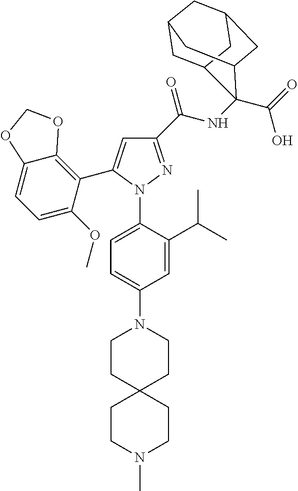 [SMILES: COc1ccc2c(c1-c1cc(C(=O)NC3(C(=O)O)C4CC5CC(C4)CC3C5)nn1-c1ccc(N3CCC4(CCN(C)CC4)CC3)cc1C(C)C)OCO2] | 724 [M + H]⁺ |
| Z | (5r,7r)-2-(1-(2- Isopropyl-4-((3- methyl-3- azaspiro[5.5]undecan- 9-yl)amino)phenyl)-5-(5- methoxybenzo[d][1,3] dioxol-4-yl)-1H- pyrazole-3- carboxamido)adamantane- 2-carboxylic acid | 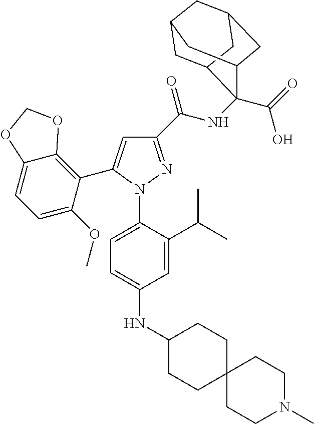 [SMILES: COc1ccc2c(c1-c1cc(C(=O)NC3(C(=O)O)C4CC5CC(C4)CC3C5)nn1-c1ccc(NC3CCC4(CC3)CCN(C)CC4)cc1C(C)C)OCO2] | 739 [M + H]⁺ |
| AA | (5r,7r)-2-(5- (enzo[c][1,2,5]thiadiazol- 4-yl)-1-(4-((3- (dimethylamino)propyl) (methyl)carbamoyl)- 2-isopropylphenyl)- 1H-pyrazole-3- carboxamido) adamantane-2- carboxylic acid | 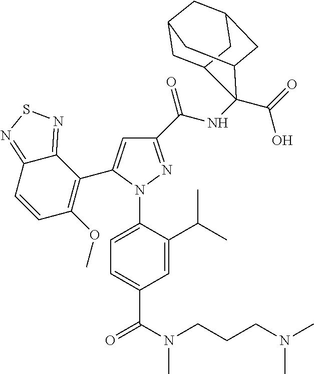 | 684 [M + H]⁺ |
| AB | (5r,7r)-2-(1-(4-(6-((3- Dimethylamino)propyl) (methyl)carbamoyl) pyridin-3-yl)-2- isopropylphenyl)-5-(5- methoxybenzo[d][1,3] dioxol-4-yl)-1H- pyrazole-3- carboxamido)adamantane- 2-carboxylic acid | 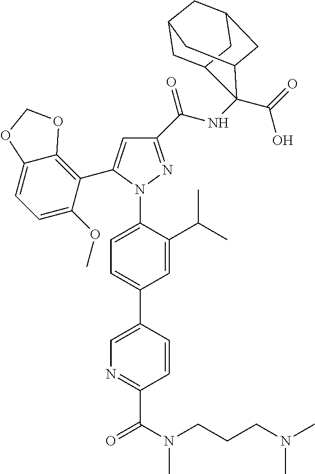 [SMILES: COc1ccc2c(c1-c1cc(C(=O)NC3(C(=O)O)C4CC5CC(C4)CC3C5)nn1-c1ccc(-c3ccc(C(=O)N(C)CCCN(C)C)nc3)cc1C(C)C)OCO2] | 777 [M + H]⁺ |
| AC | (5r,7r)-2-(1-(4-((3- (Dimethylamino)propyl) (methyl)carbamoyl)-2- isopropylphenyl)-5-(5- (trifluoromethoxy) benzo[d][1,3]dioxol-4-yl)- 1H-pyrazole-3- carboxamido) adamantane-2-carboxylic acid | 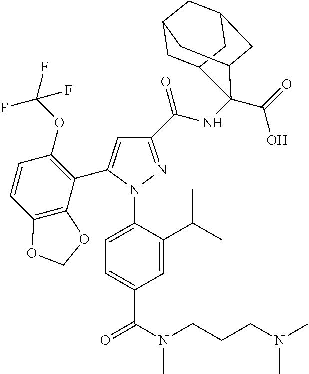 [SMILES: CC(C)c1cc(C(=O)N(C)CCCN(C)C)ccc1-n1nc(C(=O)NC2(C(=O)O)C3CC4CC(C3)CC2C4)cc1-c1c(OC(F)(F)F)ccc2c1OCO2] | 754 [M + H]⁺ |
| AD | (5r,7r)-2-(1-(4-((3- (Dimethylamino)propyl) (methyl)carbamoyl)-2- isopropylphenyl)-5-(5- fluorobenzo[d][1,3] dioxol-4-yl)- 1H-pyrazole-3- carboxamido) adamantane-2-carboxylic acid | 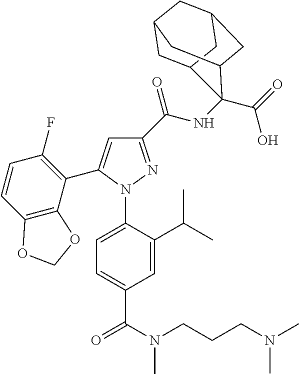 [SMILES: CC(C)c1cc(C(=O)N(C)CCCN(C)C)ccc1-n1nc(C(=O)NC2(C(=O)O)C3CC4CC(C3)CC2C4)cc1-c1c(F)ccc2c1OCO2] | 688 [M + H]⁺ |
| AE | (5r,7r)-2-(1-(4-(5-(3- (4- (Dimethylamino) butanamido)propyl)-1,3,4- thiadiazol-2-yl)-2- isopropylphenyl)-5-(5- methoxybenzo[d][1,3] dioxol-4-yl)- 1H-pyrazole-3- carboxamido) adamantane-2-carboxylic acid | 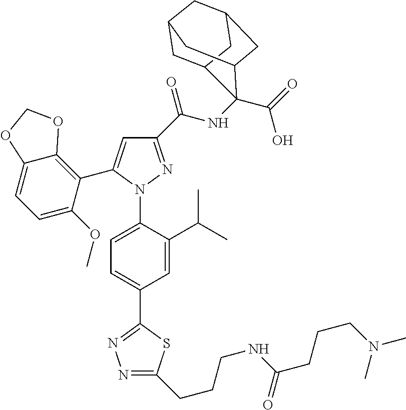 [SMILES: COc1ccc2c(c1-c1cc(C(=O)NC3(C(=O)O)C4CC5CC(C4)CC3C5)nn1-c1ccc(-c3nnc(CCCNC(=O)CCCN(C)C)s3)cc1C(C)C)OCO2] | 810 [M − H]⁻ |
| AF | (5r,7r)-2-(1-(4-(5-(3- (4- (Dimethylamino) butanamido)propyl)-1,3,4- oxadiazol-2-yl)-2- isopropylphenyl)-5-(5- methoxybenzo[d][1,3] dioxol-4-yl)- 1H-pyrazole-3- carboxamido) adamantane-2-carboxylic acid | 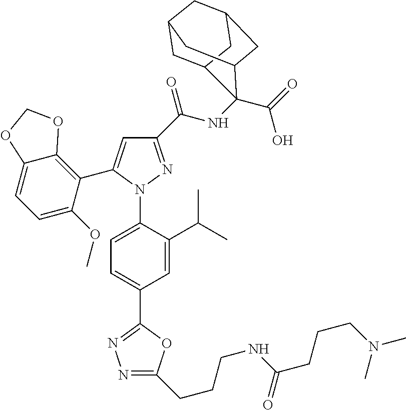 [SMILES: COc1ccc2c(c1-c1cc(C(=O)NC3(C(=O)O)C4CC5CC(C4)CC3C5)nn1-c1ccc(-c3nnc(CCCNC(=O)CCCN(C)C)o3)cc1C(C)C)OCO2] | 791 [M − H]⁻ |
| AG | (5r,7r)-2-(1-(4-(4- (Dimethylamino) butanamido)-2- isopropylphenyl)-5-(5- (trifluoromethoxy) benzo[d][1,3]dioxol- 4-yl)-1H-pyrazole-3- carboxamido) adamantane-2-carboxylic acid | 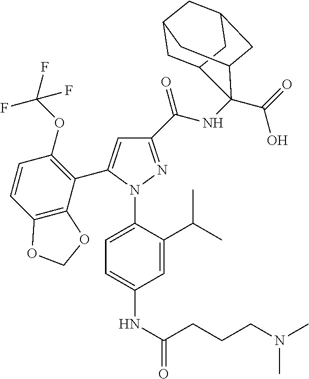 [SMILES: CC(C)c1cc(NC(=O)CCCN(C)C)ccc1-n1nc(C(=O)NC2(C(=O)O)C3CC4CC(C3)CC2C4)cc1-c1c(OC(F)(F)F)ccc2c1OCO2] | 740 [M + H]⁺ |
| AH | (5r,7r)-2-(1-(4-(3- Ethyl-5-(1- methylpiperidin-4- yl)pent-1-en-2-yl)-2- isopropylphenyl)-5-(5- methoxybenzo[d][1,3] dioxol-4-yl)-1H-pyrazole-3- carboxamido) adamantane-2-carboxylic acid | 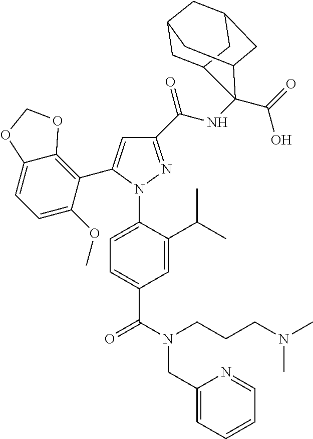 [SMILES: COc1ccc2c(c1-c1cc(C(=O)NC3(C(=O)O)C4CC5CC(C4)CC3C5)nn1-c1ccc(C(=O)N(CCCN(C)C)Cc3ccccn3)cc1C(C)C)OCO2] | 778 [M + H]⁺ |
| AI | (5r,7r)-2-(1-(4-((4-(4- (Dimethylamino) butanamido)phenyl) carbamoyl)-2- isopropylphenyl)-5-(5- methoxybenzo[d][1,3] dioxol-4-yl)-1H-pyrazole-3- carboxamido) adamantane-2-carboxylic acid | 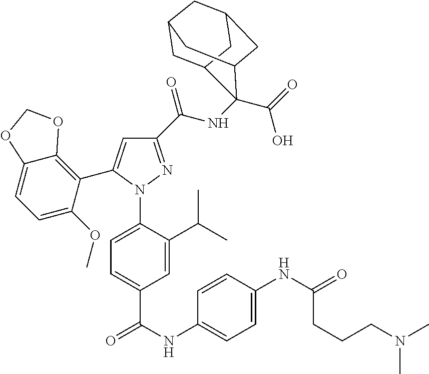 [SMILES: COc1ccc2c(c1-c1cc(C(=O)NC3(C(=O)O)C4CC5CC(C4)CC3C5)nn1-c1ccc(C(=O)Nc3ccc(NC(=O)CCCN(C)C)cc3)cc1C(C)C)OCO2] | 805 [M + H]⁺ |
| AJ | (5r,7r)-2-(1-(4-((3-(4- (Dimethylamino) butanamido)phenyl) carbamoyl)-2- isopropylphenyl)-5-(5- methoxybenzo[d][1,3] dioxol-4-yl)-1H-pyrazole-3- carboxamido) adamantane-2-carboxylic acid | 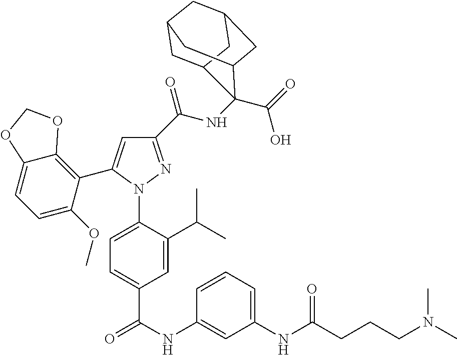 [SMILES: COc1ccc2c(c1-c1cc(C(=O)NC3(C(=O)O)C4CC5CC(C4)CC3C5)nn1-c1ccc(C(=O)Nc3cccc(NC(=O)CCCN(C)C)c3)cc1C(C)C)OCO2] | 805 [M + H]⁺ |
| AK | (5r,7r)-2-(1-(6-(3- (Dimethylamino) propoxy)-4- isopropylpyridin-3- yl)-5-(5- methoxybenzo[d][1,3] dioxol-4-yl)-1H-pyrazole-3- carboxamido) adamantane-2-carboxylic acid | 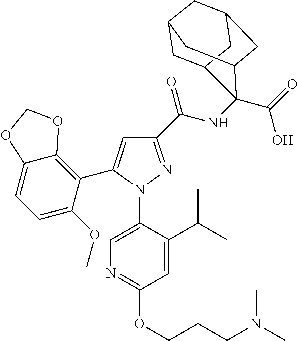 [SMILES: COc1ccc2c(c1-c1cc(C(=O)NC3(C(=O)O)C4CC5CC(C4)CC3C5)nn1-c1cnc(OCCCN(C)C)cc1C(C)C)OCO2] | 660 [M + H]⁺ |
| AL | (5r,7r)-2-(1-(4-(5-(4- (Dimethylamino) butanamido)pyridin-2-yl)- 2-isopropylphenyl)-5-(5- methoxybenzo[d][1,3] dioxol-4-yl)-1H-pyrazole-3- carboxamido) adamantane-2-carboxylic acid | 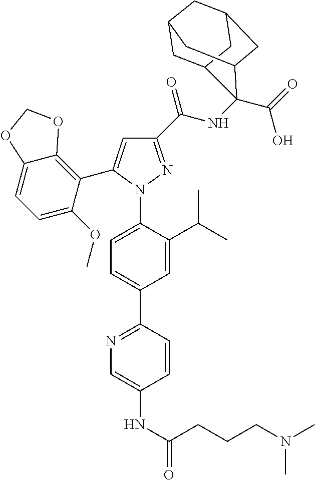 [SMILES: COc1ccc2c(c1-c1cc(C(=O)NC3(C(=O)O)C4CC5CC(C4)CC3C5)nn1-c1ccc(-c3ccc(NC(=O)CCCN(C)C)cn3)cc1C(C)C)OCO2] | 763 [M + H]⁺ |
| AM | (5r,7r)-2-(1-(4-(6-(4- (Dimethylamino) butanamido)pyridazin-3- yl)-2-isopropylphenyl)-5- (5- methoxybenzo[d][1,3] dioxol-4-yl)-1H-pyrazole-3- carboxamido) adamantane-2-carboxylic acid | 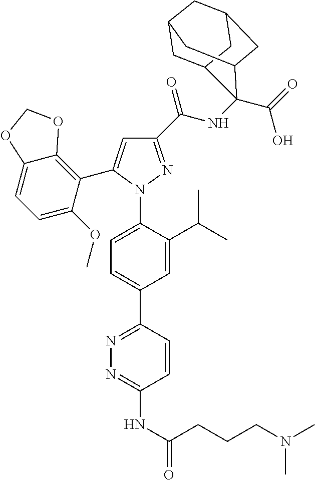 [SMILES: COc1ccc2c(c1-c1cc(C(=O)NC3(C(=O)O)C4CC5CC(C4)CC3C5)nn1-c1ccc(-c3ccc(NC(=O)CCCN(C)C)nn3)cc1C(C)C)OCO2] | 764 [M + H]⁺ |
| AN | (5r,7r)-2-(1-(2- Cyclobutyl-4-((3- (dimethylamino)propyl) (methyl)carbamoyl) phenyl)-5-(2,6- dimethoxyphenyl)-1H- pyrazole-3- carboxamido)adamantane- 2-carboxylic acid | 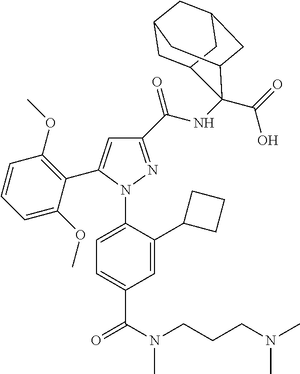 [SMILES: COc1cccc(OC)c1-c1cc(C(=O)NC2(C(=O)O)C3CC4CC(C3)CC2C4)nn1-c1ccc(C(=O)N(C)CCCN(C)C)cc1C1CCC1] | 698 [M + H]⁺ |
| AO | (5r,7r)-2-(1-(2- Isopropyl-4-(2- methyl-2H-indazol-5- yl)phenyl)-5-(5- methoxybenzo[d][1,3] dioxol-4-yl)-1H-pyrazole-3- carboxamido) adamantane-2-carboxylic acid | 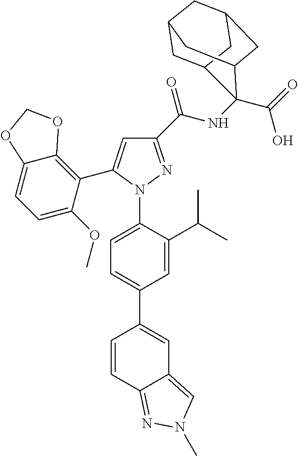 [SMILES: COc1ccc2c(c1-c1cc(C(=O)NC3(C(=O)O)C4CC5CC(C4)CC3C5)nn1-c1ccc(-c3ccc4nn(C)cc4c3)cc1C(C)C)OCO2] | 688 [M + H]⁺ |
| AP | (5r,7r)-2-(1-(2- Isopropyl-4-(1,2,3,4- tetrahydroisoquinolin- 6-yl)phenyl)-5-(5- methoxybenzo[d][1,3] dioxol-4-yl)-1H-pyrazole-3- carboxamido) adamantane-2-carboxylic acid | 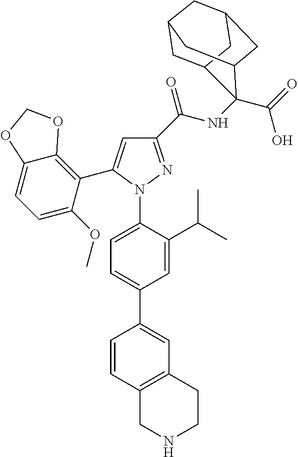 [SMILES: COc1ccc2c(c1-c1cc(C(=O)NC3(C(=O)O)C4CC5CC(C4)CC3C5)nn1-c1ccc(-c3ccc4c(c3)CCNC4)cc1C(C)C)OCO2] | 689 [M + H]⁺ |
| AQ | (5r,7r)-2-(1-(4-(7-((3- (Dimethylamino)propyl) (methyl)carbamoyl) quinolin-3-yl)-2- isopropylphenyl)-5-(5- methoxybenzo[d][1,3] dioxol-4-yl)-1H-pyrazole-3- carboxamido) adamantane-2-carboxylic acid | 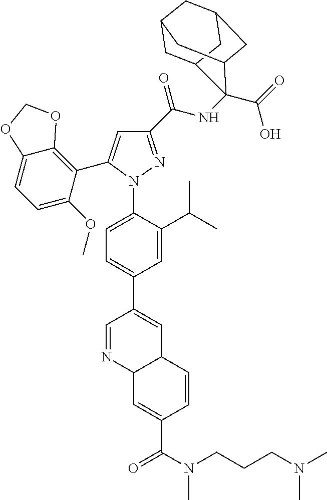 [SMILES: COc1ccc2c(c1-c1cc(C(=O)NC3(C(=O)O)C4CC5CC(C4)CC3C5)nn1-c1ccc(C3=CC4C=CC(C(=O)N(C)CCCN(C)C)=CC4N=C3)cc1C(C)C)OCO2] | 828 [M + H]⁺ |
| AR | (5r,7r)-2-(1-(2- Isopropyl-4-(4- methylpiperazin-1- yl)phenyl)-5-(5- methoxybenzo[d][1,3] dioxol-4-yl)-1H-pyrazole-3- carboxamido) adamantane-2-carboxylic acid | 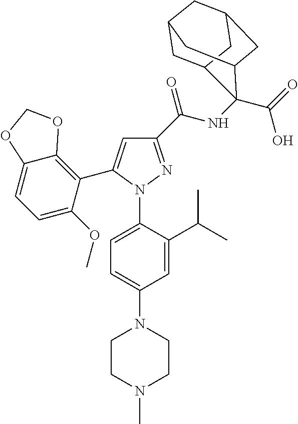 [SMILES: COc1ccc2c(c1-c1cc(C(=O)NC3(C(=O)O)C4CC5CC(C4)CC3C5)nn1-c1ccc(N3CCN(C)CC3)cc1C(C)C)OCO2] | 656 [M + H]⁺ |
| AS | (5r,7r)-2-(1-(2- Isopropyl-4-(2- methyl-1,2,3,4- tetrahydroisoquinolin- 6-yl)phenyl)-5-(5- methoxybenzo[d][1,3] dioxol-4-yl)-1H-pyrazole-3- carboxamido) adamantane-2-carboxylic acid | 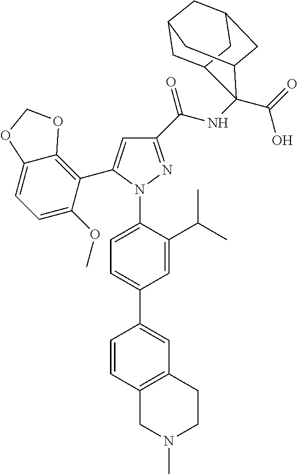 [SMILES: COc1ccc2c(c1-c1cc(C(=O)NC3(C(=O)O)C4CC5CC(C4)CC3C5)nn1-c1ccc(-c3ccc4c(c3)CCN(C)C4)cc1C(C)C)OCO2] | 703 [M + H]⁺ |
| AT | (5r,7r)-2-(1-(2- Isopropyl-4-(piperidin-4- yl)phenyl)-5-(5- methoxybenzo[d][1,3] dioxol-4-yl)-1H-pyrazole-3- carboxamido) adamantane-2-carboxylic acid | 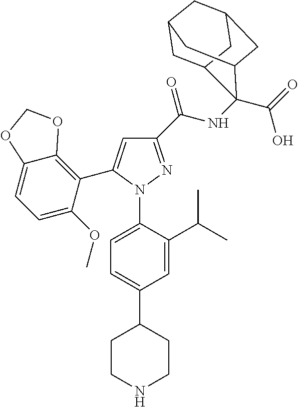 [SMILES: COc1ccc2c(c1-c1cc(C(=O)NC3(C(=O)O)C4CC5CC(C4)CC3C5)nn1-c1ccc(C3CCNCC3)cc1C(C)C)OCO2] | 641 [M + H]⁺ |
| AU | (5r,7r)-2-(1-(3′-Cyano- 4′-((3- (dimethylamino)propyl) (methyl)carbamoyl)- 3-isopropyl-[1,1′- biphenyl]-4-yl)-5-(5- methoxybenzo[d][1,3] dioxol-4-yl)-1H-pyrazole-3- carboxamido) adamantane-2-carboxylic acid | 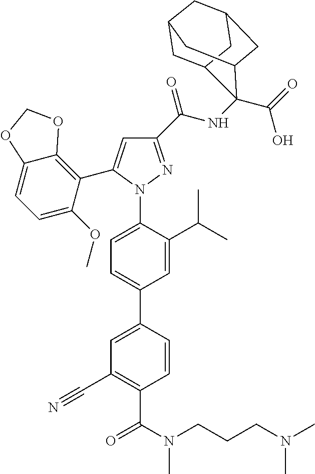 [SMILES: COc1ccc2c(c1-c1cc(C(=O)NC3(C(=O)O)C4CC5CC(C4)CC3C5)nn1-c1ccc(-c3ccc(C(=O)N(C)CCCN(C)C)c(C#N)c3)cc1C(C)C)OCO2] | 801 [M + H]⁺ |
| AV | (5r,7r)-2-(1-(2- Isopropyl-4- (piperazin-1-yl)phenyl)- 5-(5-methoxybenzo [d][1,3] dioxol-4-yl)-1H-pyrazole-3- carboxamido) adamantane-2-carboxylic acid | 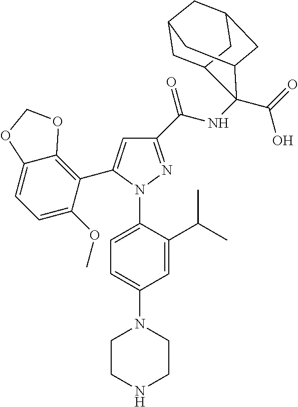 [SMILES: COc1ccc2c(c1-c1cc(C(=O)NC3(C(=O)O)C4CC5CC(C4)CC3C5)nn1-c1ccc(N3CCNCC3)cc1C(C)C)OCO2] | 642 [M + H]⁺ |
| AW | (5r,7r)-2-(1-(4′-((3- (Dimethylamino)propyl) (methyl)carbamoyl)-3- isopropyl-3′-nitro- [1,1′-biphenyl]-4-yl)-5-(5- methoxybenzo[d][1,3] dioxol-4-yl)-1H-pyrazole-3- carboxamido) adamantane-2-carboxylic acid | 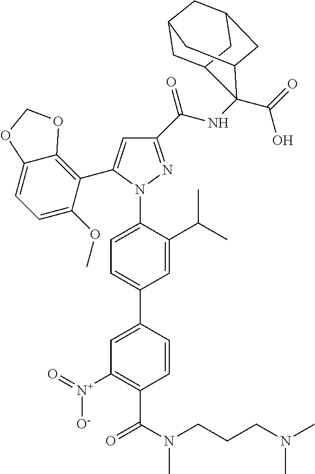 [SMILES: COc1ccc2c(c1-c1cc(C(=O)NC3(C(=O)O)C4CC5CC(C4)CC3C5)nn1-c1ccc(-c3ccc(C(=O)N(C)CCCN(C)C)c([N+](=O)[O-])c3)cc1C(C)C)OCO2] | 821 [M + H]⁺ |
| AX | (5r,7r)-2-(1-(4-(6-((3- (Dimethylamino) propyl)(methyl)carbamoyl) naphthalen-2-yl)-2- isopropylphenyl)-5-(5- methoxybenzo[d][1,3] dioxol-4-yl)-1H-pyrazole-3- carboxamido) adamantane-2-carboxylic acid | 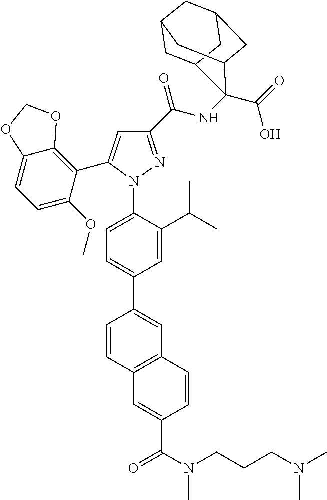 [SMILES: COc1ccc2c(c1-c1cc(C(=O)NC3(C(=O)O)C4CC5CC(C4)CC3C5)nn1-c1ccc(-c3ccc4cc(C(=O)N(C)CCCN(C)C)ccc4c3)cc1C(C)C)OCO2] | 827 [M + H]⁺ |
| AY | (5r,7r)-2-(1-(2- Isopropyl-4- (isoquinolin-3-yl)phenyl)- 5-(5-methoxybenzo[d][1,3] dioxol-4-yl)-1H-pyrazole-3- carboxamido) adamantane-2-carboxylic acid | 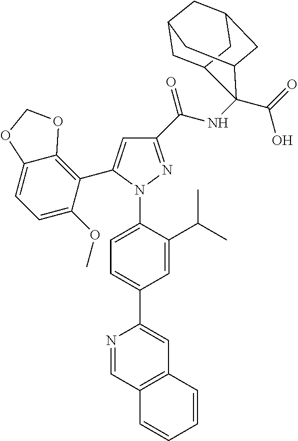 [SMILES: COc1ccc2c(c1-c1cc(C(=O)NC3(C(=O)O)C4CC5CC(C4)CC3C5)nn1-c1ccc(-c3cc4ccccc4cn3)cc1C(C)C)OCO2] | 685 [M + H]⁺ |
| AZ | (5r,7r)-2-(1-(2- Isopropyl-4-(quinolin- 3-yl)phenyl)-5-(5- methoxybenzo[d][1,3] dioxol-4-yl)-1H-pyrazole-3- carboxamido) adamantane-2-carboxylic acid | 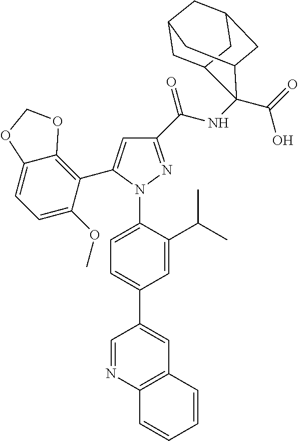 [SMILES: COc1ccc2c(c1-c1cc(C(=O)NC3(C(=O)O)C4CC5CC(C4)CC3C5)nn1-c1ccc(-c3cnc4ccccc4c3)cc1C(C)C)OCO2] | 685 [M + H]⁺ |
| BA | (5r,7r)-2-(1-(4′-((3- (Dimethylamino)propyl) (methyl)carbamoyl)-3- isopropyl-3′-methoxy- [1,1′-biphenyl]-4-yl)-5-(5- methoxybenzo[d][1,3] dioxol-4-yl)-1H-pyrazole-3- carboxamido) adamantane-2-carboxylic acid | 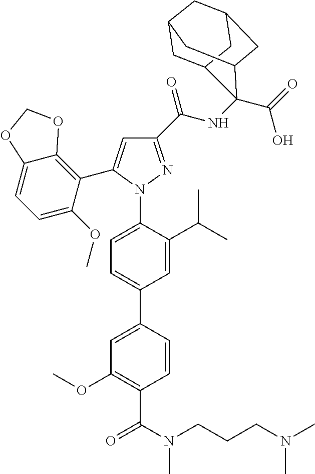 [SMILES: COc1cc(-c2ccc(-n3nc(C(=O)NC4(C(=O)O)C5CC6CC(C5)CC4C6)cc3-c3c(OC)ccc4c3OCO4)c(C(C)C)c2)ccc1C(=O)N(C)CCCN(C)C] | 806 [M + H]⁺ |
| BB | (5r,7r)-2-(1-(4-(1-(3- (Dimethylamino)propyl)- 1H-indazol-3-yl)-2- isopropylphenyl)-5-(5- methoxybenzo[d][1,3] dioxol-4-yl)-1H-pyrazole-3- carboxamido) adamantane-2-carboxylic acid | 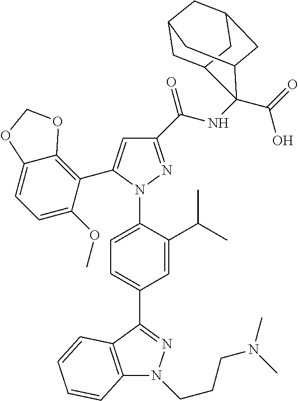 [SMILES: COc1ccc2c(c1-c1cc(C(=O)NC3(C(=O)O)C4CC5CC(C4)CC3C5)nn1-c1ccc(-c3nn(CCCN(C)C)c4ccccc34)cc1C(C)C)OCO2] | 759 [M + H]⁺ |
| BC | (5r,7r)-2-(1-(4-(3-((3- (Dimethylamino)propyl) (methyl)carbamoyl) quinolin-7-yl)-2- isopropylphenyl)-5-(5- methoxybenzo[d][1,3] dioxol-4-yl)-1H-pyrazole-3- carboxamido) adamantane-2-carboxylic acid | 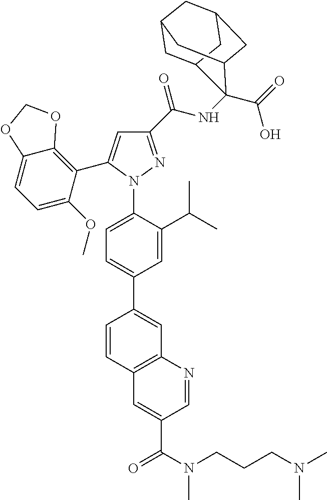 [SMILES: COc1ccc2c(c1-c1cc(C(=O)NC3(C(=O)O)C4CC5CC(C4)CC3C5)nn1-c1ccc(-c3ccc4cc(C(=O)N(C)CCCN(C)C)cnc4c3)cc1C(C)C)OCO2] | 828 [M + H]⁺ |
| BD | (5r,7r)-2-(1-(4-((4-(4- (Dimethylamino) butanamido)phenyl)amino)- 2-isopropylphenyl)-5- (5-methoxybenzo[d][1,3] dioxol-4-yl)-1H-pyrazole-3- carboxamido) adamantane-2-carboxylic acid | 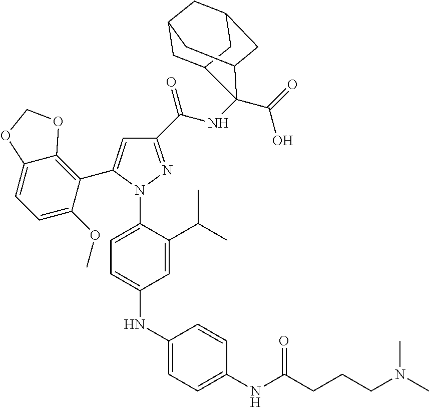 [SMILES: COc1ccc2c(c1-c1cc(C(=O)NC3(C(=O)O)C4CC5CC(C4)CC3C5)nn1-c1ccc(Nc3ccc(NC(=O)CCCN(C)C)cc3)cc1C(C)C)OCO2] | 778 [M + H]⁺ |
| BE | (5r,7r)-2-(1-(4-(4-(4- (Dimethylamino) butanamido)phenoxy)-2- isopropylphenyl)-5-(5- methoxybenzo[d][1,3] dioxol-4-yl)-1H-pyrazole-3- carboxamido) adamantane-2-carboxylic acid | 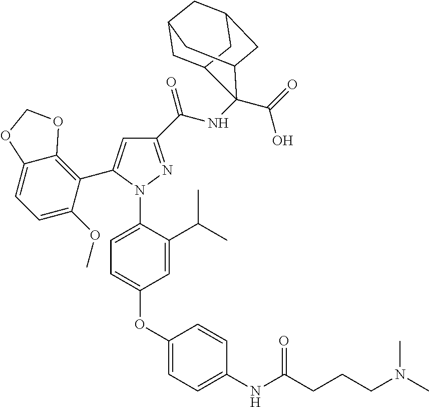 [SMILES: COc1ccc2c(c1-c1cc(C(=O)NC3(C(=O)O)C4CC5CC(C4)CC3C5)nn1-c1ccc(Oc3ccc(NC(=O)CCCN(C)C)cc3)cc1C(C)C)OCO2] | 779 [M + H]⁺ |
| BF | (5r,7r)-2-(1-(2- Isopropyl-4-(1- methyl-1,2,3,6- tetrahydropyridin-4- yl)phenyl)-5-(5- methoxybenzo[d][1,3] dioxol-4-yl)-1H-pyrazole-3- carboxamido) adamantane-2-carboxylic acid | 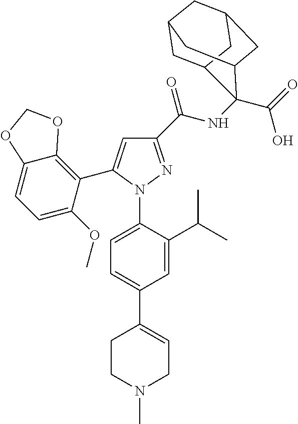 [SMILES: COc1ccc2c(c1-c1cc(C(=O)NC3(C(=O)O)C4CC5CC(C4)CC3C5)nn1-c1ccc(C3=CCN(C)CC3)cc1C(C)C)OCO2] | 653 [M + H]⁺ |
| BG | (5r,7r)-2-(1-(2- Isopropyl-4-(1- methylpiperidin-4- yl)phenyl)-5-(5- methoxybenzo[d][1,3] dioxol-4-yl)-1H-pyrazole-3- carboxamido) adamantane-2-carboxylic acid | 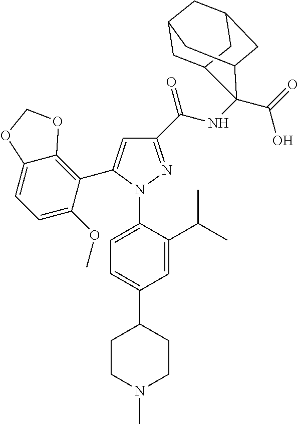 [SMILES: COc1ccc2c(c1-c1cc(C(=O)NC3(C(=O)O)C4CC5CC(C4)CC3C5)nn1-c1ccc(C3CCN(C)CC3)cc1C(C)C)OCO2] | 655 [M + H]⁺ |
| BH | (5r,7r)-2-(1-(4-((3- (Dimethylamino) propyl)(methyl)carbamoyl)- 2-isopropylphenyl)-5- (5-(2- methoxyethoxy) benzo[d][1,3] dioxol-4-yl)-1H-pyrazole-3- carboxamido) adamantane-2-carboxylic acid | 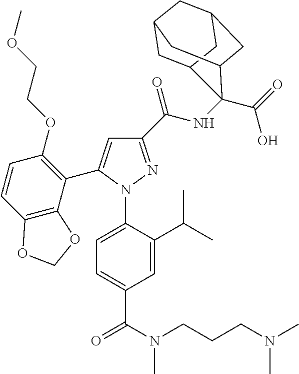 [SMILES: COCCOc1ccc2c(c1-c1cc(C(=O)NC3(C(=O)O)C4CC5CC(C4)CC3C5)nn1-c1ccc(C(=O)N(C)CCCN(C)C)cc1C(C)C)OCO2] | 645 [M + H]⁺ |
| BI | (5r,7r)-2-(5-(5- Cyanobenzo[d][1,3] dioxol-4-yl)-1-(4-((3- (dimethylamino)propyl) (methyl)carbamoyl)-2- isopropylphenyl)-1H- pyrazole-3-carboxamido) adamantane-2-carboxylic acid | 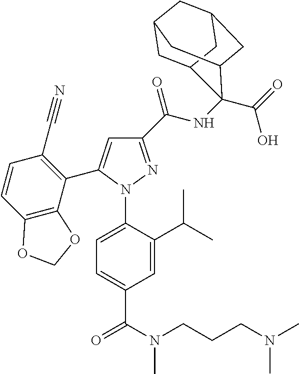 [SMILES: CC(C)c1cc(C(=O)N(C)CCCN(C)C)ccc1-n1nc(C(=O)NC2(C(=O)O)C3CC4CC(C3)CC2C4)cc1-c1c(C#N)ccc2c1OCO2] | 695 [M + H]⁺ |

### Example BJ

### N-((5r,7r)-2-(1H-Tetrazol-5-yl)adamantan-2-yl)-1-(4-(4-(dimethylamino)butanamido)-2-isopropylphenyl)-5-(5-methoxybenzo[d][1,3]dioxol-4-yl)-1H-pyrazole-3-carboxamide

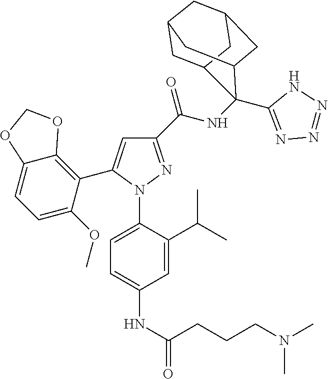 [SMILES: COc1ccc2c(c1-c1cc(C(=O)NC3(c4nnn[nH]4)C4CC5CC(C4)CC3C5)nn1-c1ccc(NC(=O)CCCN(C)C)cc1C(C)C)OCO2]

A soln of 10 wt % Pd/C (43.23 mg, 0.041 mmol) and N-[2-(1-benzyl-1,2,3,4-tetrazol-5-yl)adamantan-2-yl]-1-{4-[4-(dimethylamino)butanamido]-2-isopropylphenyl}-5-(5-methoxy-2H-1,3-benzodioxol-4-yl)pyrazole-3-carboxamide (65 mg, 0.081 mmol) in methanol (5 mL) was stirred at RT for 2 h under H₂. The mixture was filtered and the filter cake washed with MeOH (2×10 mL). The filtrate was concentrated and Prep-HPLC with the following conditions: Column, XBridge Shield RP18 OBD 30*150 mm, 5 μm; Mobile Phase, 45 to 55% MeOH in H₂O (0.1% FA) to afford title compound (5.4 mg, 9%) as a white solid. MS ES+ m/z 711 [M+H]⁺.

### Example BK

### N-((5r,7r)-2-carbamoyladamantan-2-yl)-1-(4-(4-(dimethylamino)butanamido)-2-isopropylphenyl)-5-(5-methoxybenzo[d][1,3]dioxol-4-yl)-1H-pyrazole-3-carboxamide; formic acid

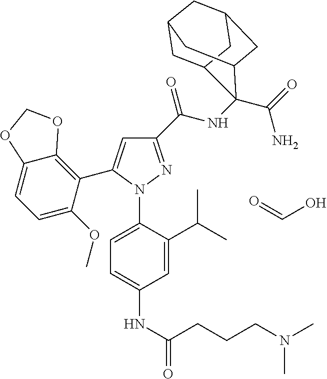

A soln of 2-(1-{4-[4-(dimethylamino)butanamido]-2-isopropylphenyl}-5-(5-methoxy-2H-1,3-benzodioxol-4-yl)pyrazole-3-amido)adamantane-2-carboxylic acid (340 mg, 0.5 mmol) and HATU (282 mg, 0.74 mmol) in DMF (4 mL) was treated with DIEA (448 mg, 3.47 mmol) and NH₄OMe (0.45 mL, 3.13 mmol). After stirring a RT ON under N₂, the mixture was concentrated and purified by reversed-phase flash chromatography with the following conditions: column, C18; mobile phase, 10% to 100% ACN in H₂O (0.1% FA) to provide title compound (170 mg, 35%). MS ES+ m/z 686 [M+H]⁺.

The compounds in Table 58 were prepared and the MS data is recorded below. These compounds can be prepared according to Schemes 1-3 or the foregoing Preparations and Examples. Optimum reaction conditions can vary with the particular reactants or solvents used, but such conditions can be determined by one skilled in the art.

TABLE 58

| | | | MS ES+ |
|---|---|---|---|
| Ex # | Chemical Name | Structure | m/z |
| BL | (5r,7r)-2-(1-(3- Cyclopropyl-5-(3- (dimethylamino)propo- xy)pyrazin-2-yl)-5-(5- methoxybenzo[d][1,3] dioxol-4-yl)-1H- pyrazole-3- carboxamido)adaman- tane-2-carboxylic acid | 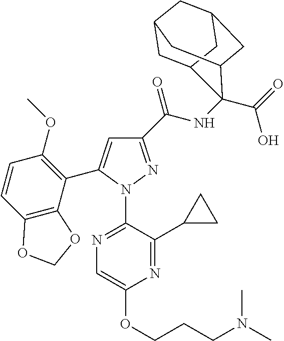 [SMILES: COc1ccc2c(c1-c1cc(C(=O)NC3(C(=O)O)C4CC5CC(C4)CC3C5)nn1-c1ncc(OCCCN(C)C)nc1C1CC1)OCO2] | 659 [M + H]⁺ |
| BM | (5r,7r)-2-(1-(6-((3- (Dimethylamino)pro- pyl)(methyl)amino)-4- isopropylpyridazin-3- yl)-5-(5- methoxybenzo[d][1,3] dioxol-4-yl)-1H- pyrazole-3- carboxamido)adaman- tane-2-carboxylic acid | 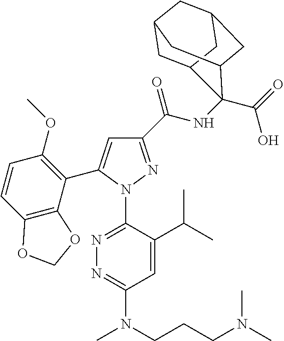 | 702 [M + H]⁺ |
| BN | (5r,7r)-2-(1-(3- Cyclopropyl-5-(3- (dimethylamino)propo- xy)pyridin-2-yl)-5-(5- methoxybenzo[d][1,3] dioxol-4-yl)-1H- pyrazole-3- carboxamido)adaman- tane-2-carboxylic acid | 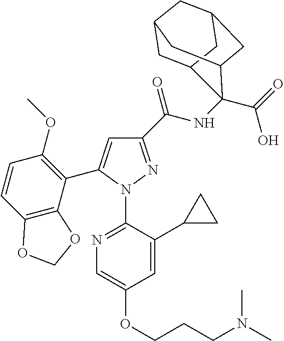 [SMILES: COc1ccc2c(c1-c1cc(C(=O)NC3(C(=O)O)C4CC5CC(C4)CC3C5)nn1-c1ncc(OCCCN(C)C)cc1C1CC1)OCO2] | 659 [M + H]⁺ |
| BO | (5r,7r)-2-(1-(4-((3- (Dimethylamino)pro- pyl)amino)-2- isopropylphenyl)-5-(5- methoxybenzo[d][1,3] dioxol-4-yl)-1H- pyrazole-3- carboxamido)adaman- tane-2-carboxylic acid | 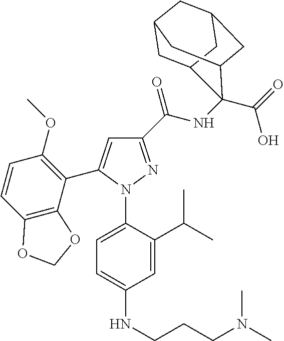 [SMILES: COc1ccc2c(c1-c1cc(C(=O)NC3(C(=O)O)C4CC5CC(C4)CC3C5)nn1-c1ccc(NCCCN(C)C)cc1C(C)C)OCO2] | 335 [M/2 + H]⁺ |
| BP | (5r,7r)-2-(1-(4-((3- (Dimethylamino)pro- pyl)carbamoyl)-2- isopropylphenyl)-5-(5- methoxybenzo[d][1,3] dioxol-4-yl)-1H- pyrazole-3- carboxamido)adaman- tane-2-carboxylic acid | 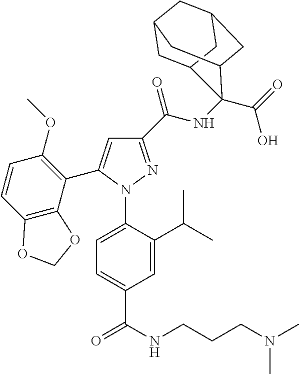 | 695 [M + H]⁺ |
| BQ | (5r,7r)-2-(1-(4-((3- (Dimethylamino)pro- pyl)carbamoyl)-2- isopropylphenyl)-5-(6- methoxy- [1,3]dioxolo[4,5- c]pyridin-7-yl)-1H- pyrazole-3- carboxamido)adaman- tane-2-carboxylic acid | 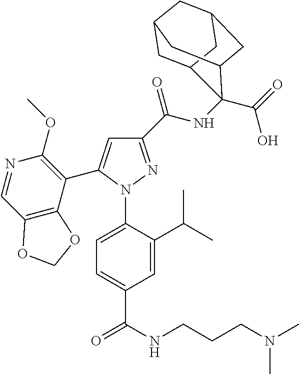 | 702 [M + H]⁺ |
| BR | (5r,7r)-2-(1-(3-(3- (Dimethylamino)pro- poxy)-2- isopropylphenyl)-5-(5- methoxybenzo[d][1,3] dioxol-4-yl)-1H- pyrazole-3- carboxamido)adaman- tane-2-carboxylic acid | 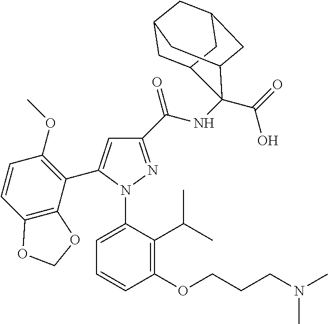 [SMILES: COc1ccc2c(c1-c1cc(C(=O)NC3(C(=O)O)C4CC5CC(C4)CC3C5)nn1-c1cccc(OCCCN(C)C)c1C(C)C)OCO2] | 659 [M + H]⁺ |

### Example 1

### 2,2′,2″-(10-(2-((3-((3-(4-(3-(((5r,7r)-2-Carboxyadamantan-2-yl)carbamoyl)-5-(5-methoxybenzo[d][1,3]dioxol-4-yl)-1H-pyrazol-1-yl)-3-isopropylphenoxy)propyl)(methyl)amino)propyl)(methyl)amino)-2-oxoethyl)-1,4,7,10-tetraazacyclododecane-1,4,7-triyl)triacetic acid

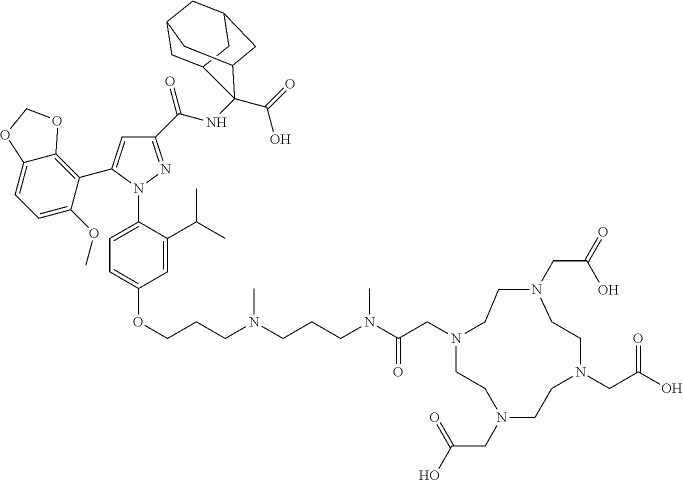 [SMILES: COc1ccc2c(c1-c1cc(C(=O)NC3(C(=O)O)C4CC5CC(C4)CC3C5)nn1-c1ccc(OCCCN(C)CCCN(C)C(=O)CN3CCN(CC(=O)O)CCN(CC(=O)O)CCN(CC(=O)O)CC3)cc1C(C)C)OCO2]

A soln of tri-tert-butyl 2,2′,2″-(10-(2-((3-((3-(4-(3-(((5r,7r)-2-(tert-butoxycarbonyl)adamantan-2-yl)carbamoyl)-5-(5-methoxybenzo[d][1,3]dioxol-4-yl)-1H-pyrazol-1-yl)-3-isopropylphenoxy)propyl)(methyl)amino)propyl)(methyl)amino)-2-oxoethyl)-1,4,7,10-tetraazacyclododecane-1,4,7-triyl)triacetate (20 mg, 0.015 mmol), TIPS (2.4 mg, 0.015 mmol) in DCM (1 mL) was treated with TFA (1 mL). After stirring at 45° C. for 3 h the mixture was concentrated under vacuum to afford crude title compound (17 mg, 100%) as a colorless foam. MS ES+ m/z 1103 [M+H]⁺.

The following compounds were prepared in a manner essentially analogous to the preparation method of 2,2′,2″-(10-(2-((3-((3-(4-(3-(((5r,7r)-2-Carboxyadamantan-2-yl)carbamoyl)-5-(5-methoxybenzo[d][1,3]dioxol-4-yl)-1H-pyrazol-1-yl)-3-isopropylphenoxy)propyl)(methyl)amino)propyl)(methyl)amino)-2-oxoethyl)-1,4,7,10-tetraazacyclododecane-1,4,7-triyl)triacetic acid using the appropriate reagents, adjusting temperature, adjusting reaction time to determine completion of the reaction, and adjusting the purification system as appropriate.

TABLE 20

| | | | MS |
|---|---|---|---|
| | | | ES+ |
| Ex # | Chemical Name | Structure | m/z |
| 2 | 2,2′,2″-(10-(2-((2-((3- (4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenoxy)pro- pyl)(methyl)amino)eth- yl)amino)-2- oxoethyl)-1,4,7,10- tetraazacyclododecane- 1,4,7-triyl)triacetic acid | 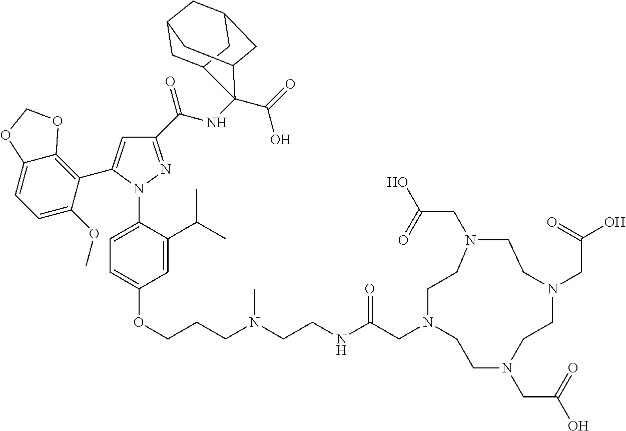 [SMILES: COc1ccc2c(c1-c1cc(C(=O)NC3(C(=O)O)C4CC5CC(C4)CC3C5)nn1-c1ccc(OCCCN(C)CCNC(=O)CN3CCN(CC(=O)O)CCN(CC(=O)O)CCN(CC(=O)O)CC3)cc1C(C)C)OCO2] | 1075 [M + H]⁺ |
| 3 | 2,2′,2″-(10-(2-((4-((2- ((3-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenoxy)pro- pyl)(methyl)amino)eth- yl)amino)-4- oxobutyl)(methyl)ami- no)-2-oxoethyl)- 1,4,7,10- tetraazacyclododecane- 1,4,7-triyl)triacetic acid | 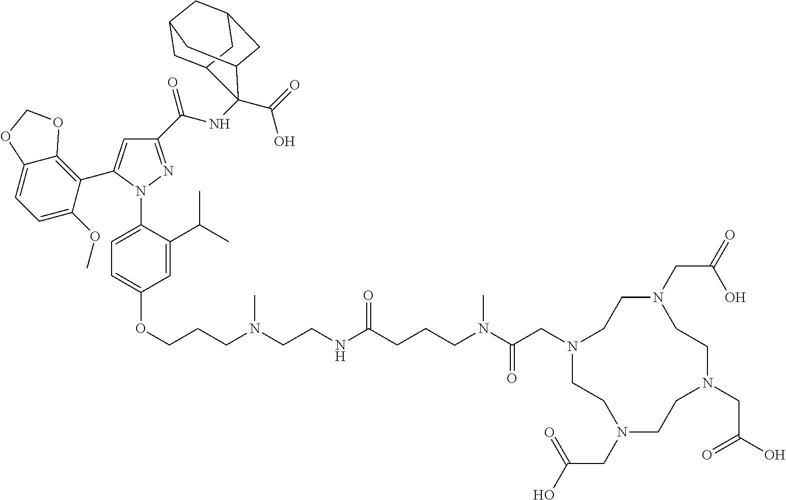 [SMILES: COc1ccc2c(c1-c1cc(C(=O)NC3(C(=O)O)C4CC5CC(C4)CC3C5)nn1-c1ccc(OCCCN(C)CCNC(=O)CCCN(C)C(=O)CN3CCN(CC(=O)O)CCN(CC(=O)O)CCN(CC(=O)O)CC3)cc1C(C)C)OCO2] | 1174 [M + H]⁺ |
| 4 | 2,2′,2″-(10-(2-((3-((4- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)amino)- 4- oxobutyl)(methyl)ami- no)propyl)(methyl)ami- no)-2-oxoethyl)- 1,4,7,10- tetraazacyclododecane- 1,4,7-triyl)triacetic acid | 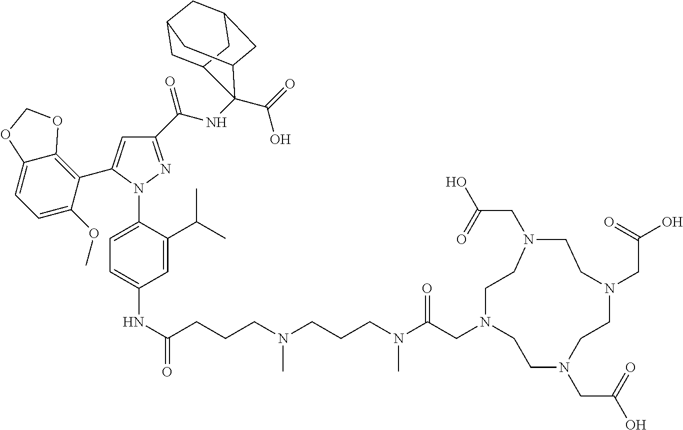 [SMILES: COc1ccc2c(c1-c1cc(C(=O)NC3(C(=O)O)C4CC5CC(C4)CC3C5)nn1-c1ccc(NC(=O)CCCN(C)CCCN(C)C(=O)CN3CCN(CC(=O)O)CCN(CC(=O)O)CCN(CC(=O)O)CC3)cc1C(C)C)OCO2] | 1130 [M + H]⁺ |
| 5 | 2,2′,2″-(10-(2-((3-((3- ((N-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)sulfa- moyl)(methyl)amino)pro- pyl)(methyl)amino)pro- pyl)(methyl)amino)- 2-oxoethyl)-1,4,7,10- tetraazacyclododecane- 1,4,7-triyl)triacetic acid | 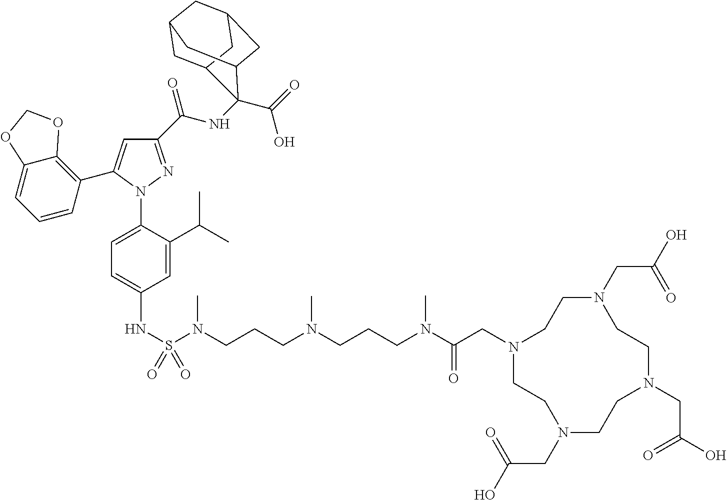 | 1195 [M + H]⁺ |
| 6 | 2,2′,2″-(10-(2-((3-((4- ((4′-(3-(((5r,7r)-2- Carboxyadamantan-2- yl)carbamoyl)-5-(5- methoxybenzo[d][1,3] dioxol-4-yl)-1H- pyrazol-1-yl)-3′- isopropyl-[1,1′- biphenyl]-4- yl)amino)-4- oxobutyl)(methyl)ami- no)propyl)(methyl)ami- no)-2-oxoethyl)- 1,4,7,10- tetraazacyclododecane- 1,4,7-triyl)triacetic acid | 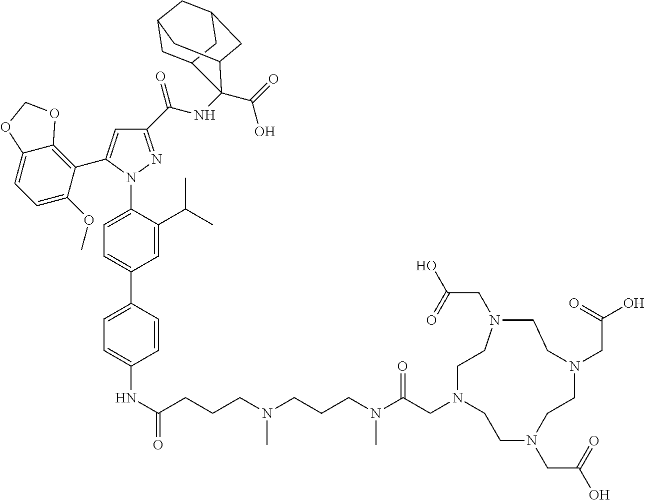 [SMILES: COc1ccc2c(c1-c1cc(C(=O)NC3(C(=O)O)C4CC5CC(C4)CC3C5)nn1-c1ccc(-c3ccc(NC(=O)CCCN(C)CCCN(C)C(=O)CN4CCN(CC(=O)O)CCN(CC(=O)O)CCN(CC(=O)O)CC4)cc3)cc1C(C)C)OCO2] | 1205 [M + H]⁺ |
| 7 | 2,2′,2″-(10-(2-((3-((3- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)(meth- yl)amino)propyl)(meth- yl)amino)propyl)(meth- yl)amino)-2- oxoethyl)-1,4,7,10- tetraazacyclododecane- 1,4,7-triyl)triacetic acid | 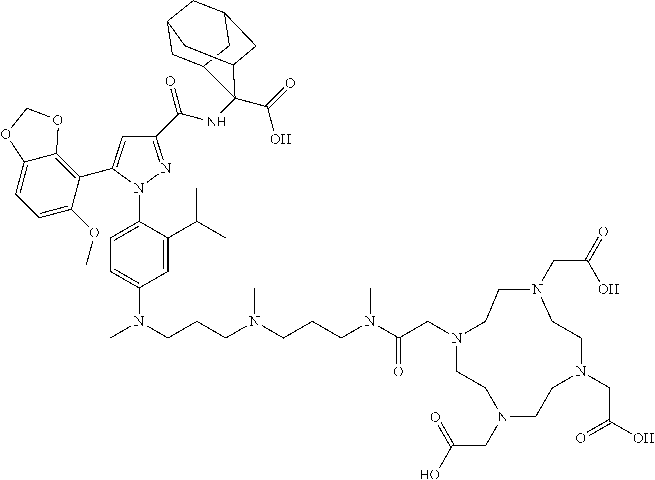 [SMILES: COc1ccc2c(c1-c1cc(C(=O)NC3(C(=O)O)C4CC5CC(C4)CC3C5)nn1-c1ccc(N(C)CCCN(C)CCCN(C)C(=O)CN3CCN(CC(=O)O)CCN(CC(=O)O)CCN(CC(=O)O)CC3)cc1C(C)C)OCO2] | 1116 [M + H]⁺ |
| 8 | 2,2′,2″-(10-(2-((3-((3- (3-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-1- methylureido)propyl)(meth- yl)amino)propyl)(meth- yl)amino)-2- oxoethyl)-1,4,7,10- tetraazacyclododecane- 1,4,7-triyl)triacetic acid | 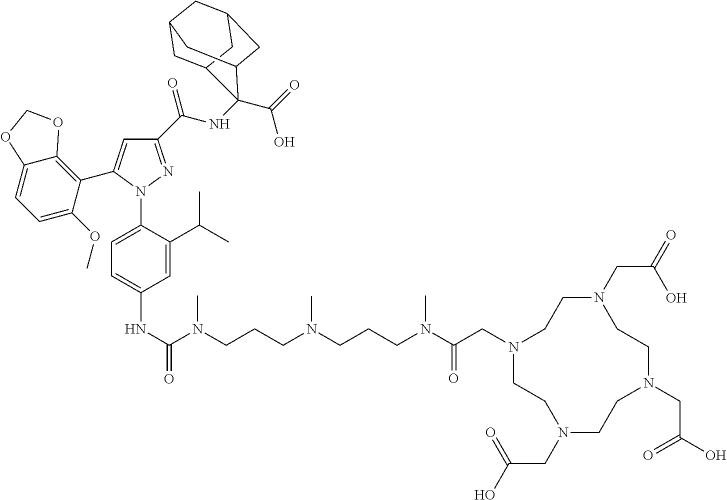 [SMILES: COc1ccc2c(c1-c1cc(C(=O)NC3(C(=O)O)C4CC5CC(C4)CC3C5)nn1-c1ccc(NC(=O)N(C)CCCN(C)CCCN(C)C(=O)CN3CCN(CC(=O)O)CCN(CC(=O)O)CCN(CC(=O)O)CC3)cc1C(C)C)OCO2] | 1159 [M + H]⁺ |
| 9 | 2,2′,2″-(10-(2-((3-((3- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)amino) propyl)(methyl)amino) propyl)amino)-2- oxoethyl)-1,4,7,10- tetraazacyclododecane- 1,4,7-triyl)triacetic acid | 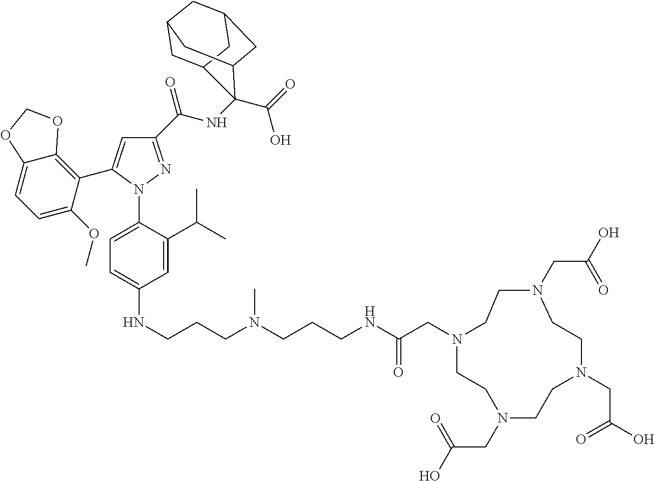 [SMILES: COc1ccc2c(c1-c1cc(C(=O)NC3(C(=O)O)C4CC5CC(C4)CC3C5)nn1-c1ccc(NCCCN(C)CCCNC(=O)CN3CCN(CC(=O)O)CCN(CC(=O)O)CCN(CC(=O)O)CC3)cc1C(C)C)OCO2] | 1088 [M + H]⁺ |
| 10 | 2,2′,2″-(10-(2-((3-((3- (((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)carba- moyl)oxy)propyl)(meth- yl)amino)propyl)(meth- yl)amino)-2- oxoethyl)-1,4,7,10- tetraazacyclododecane- 1,4,7-triyl)triacetic acid | 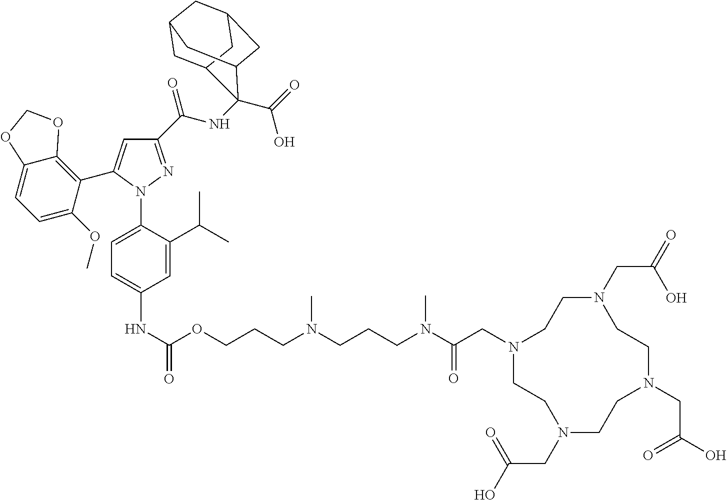 [SMILES: COc1ccc2c(c1-c1cc(C(=O)NC3(C(=O)O)C4CC5CC(C4)CC3C5)nn1-c1ccc(NC(=O)OCCCN(C)CCCN(C)C(=O)CN3CCN(CC(=O)O)CCN(CC(=O)O)CCN(CC(=O)O)CC3)cc1C(C)C)OCO2] | 1146 [M + H]⁺ |
| 11 | 2,2′,2″-(10-(2-((3-((3- (3-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)ureido) propyl)(methyl)amino) propyl)(methyl)amino)- 2-oxoethyl)- 1,4,7,10- tetraazacyclododecane- 1,4,7-triyl)triacetic acid | 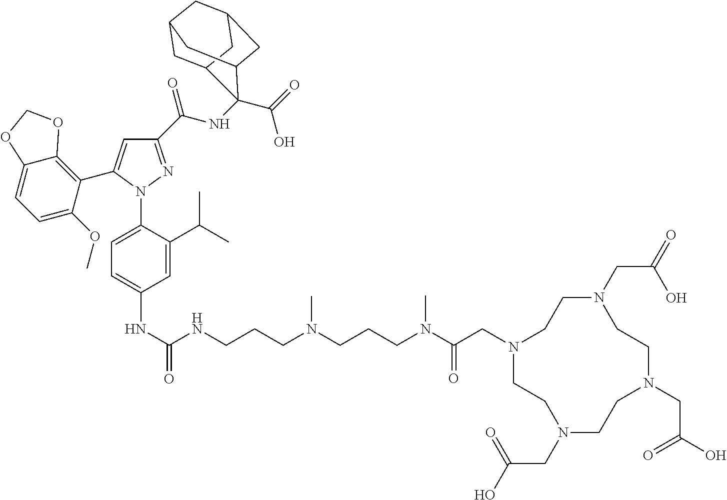 [SMILES: COc1ccc2c(c1-c1cc(C(=O)NC3(C(=O)O)C4CC5CC(C4)CC3C5)nn1-c1ccc(NC(=O)NCCCN(C)CCCN(C)C(=O)CN3CCN(CC(=O)O)CCN(CC(=O)O)CCN(CC(=O)O)CC3)cc1C(C)C)OCO2] | 1145 [M + H]⁺ |
| 12 | 2,2′,2″-(10-(2-((3-((3- (4-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-1H- 1,2,3-triazol-1- yl)propyl)(methyl)ami- no)propyl)(methyl)ami- no)-2-oxoethyl)- 1,4,7,10- tetraazacyclododecane- 1,4,7-triyl)triacetic acid | 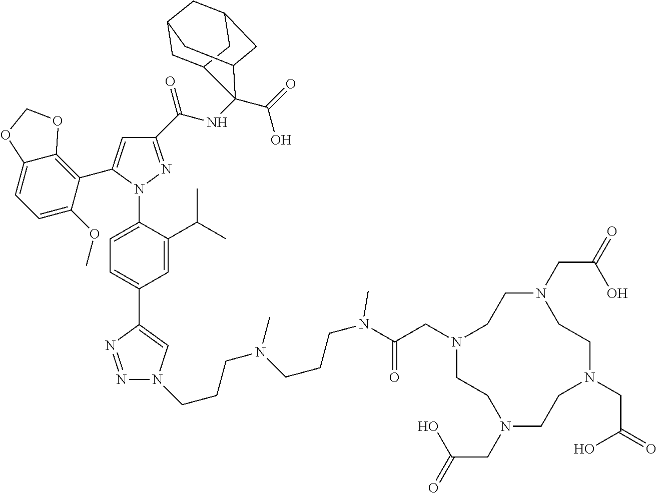 [SMILES: COc1ccc2c(c1-c1cc(C(=O)NC3(C(=O)O)C4CC5CC(C4)CC3C5)nn1-c1ccc(-c3cn(CCCN(C)CCCN(C)C(=O)CN4CCN(CC(=O)O)CCN(CC(=O)O)CCN(CC(=O)O)CC4)nn3)cc1C(C)C)OCO2] | 1154 [M + H]⁺ |
| 13 | 2,2′,2″-(10-(2-((3-((4- ((4-(3-(((5r,7r)-2- carboxyadamantan-2- yl)carbamoyl)-5-(5- methoxybenzo[d][1,3] dioxol-4-yl)-1H- pyrazol-1-yl)-3- isopropylphenyl)amino)- 4- oxobutyl)(methyl)ami- no)propyl)amino)-2- oxoethyl)-1,4,7,10- tetraazacyclododecane- 1,4,7-triyl)triacetic acid | 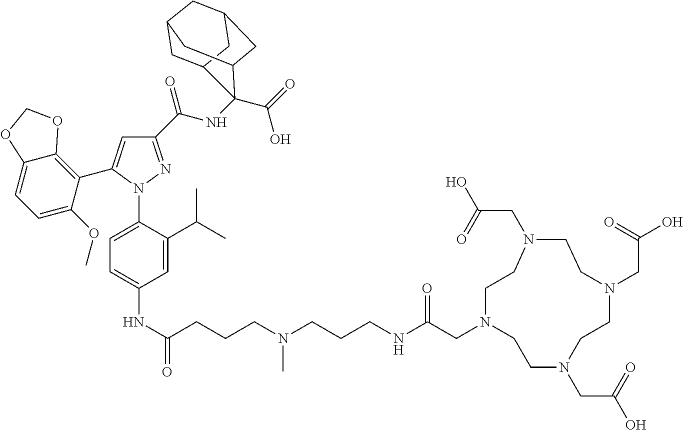 [SMILES: COc1ccc2c(c1-c1cc(C(=O)NC3(C(=O)O)C4CC5CC(C4)CC3C5)nn1-c1ccc(NC(=O)CCCN(C)CCCNC(=O)CN3CCN(CC(=O)O)CCN(CC(=O)O)CCN(CC(=O)O)CC3)cc1C(C)C)OCO2] | 1116 [M + H]⁺ |
| 14 | 2,2′,2″-(10-(2-((3-((3- (4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenoxy)pro- pyl)(methyl)amino)pro- pyl)amino)-2- oxoethyl)-1,4,7,10- tetraazacyclododecane- 1,4,7-triyl)triacetic acid | 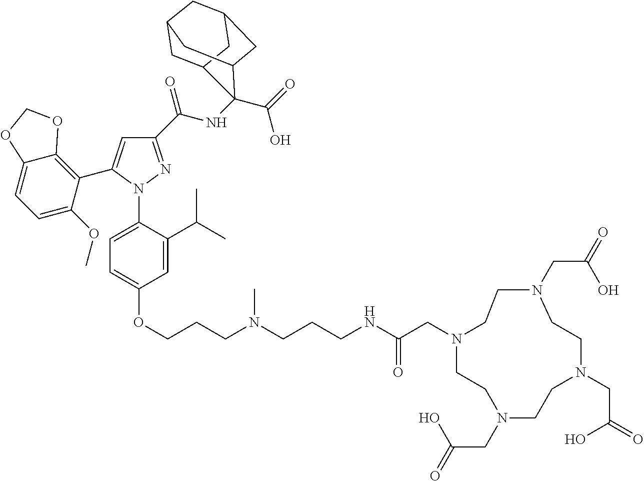 [SMILES: COc1ccc2c(c1-c1cc(C(=O)NC3(C(=O)O)C4CC5CC(C4)CC3C5)nn1-c1ccc(OCCCN(C)CCCNC(=O)CN3CCN(CC(=O)O)CCN(CC(=O)O)CCN(CC(=O)O)CC3)cc1C(C)C)OCO2] | 1089 [M + H]⁺ |
| 15 | 2,2′,2″-(10-(17-((4-(3- (((5r,7r)-2- Carboxyadamantan-2- yl)carbamoyl)-5-(5- methoxybenzo[d][1,3] dioxol-4-yl)-1H- pyrazol-1-yl)-3- isopropylphenyl)amino)- 2,17-dioxo- 6,9,12,15-tetraoxa-3- azaheptadecyl)- 1,4,7,10- tetraazacyclododecane- 1,4,7-triyl)triacetic acid | 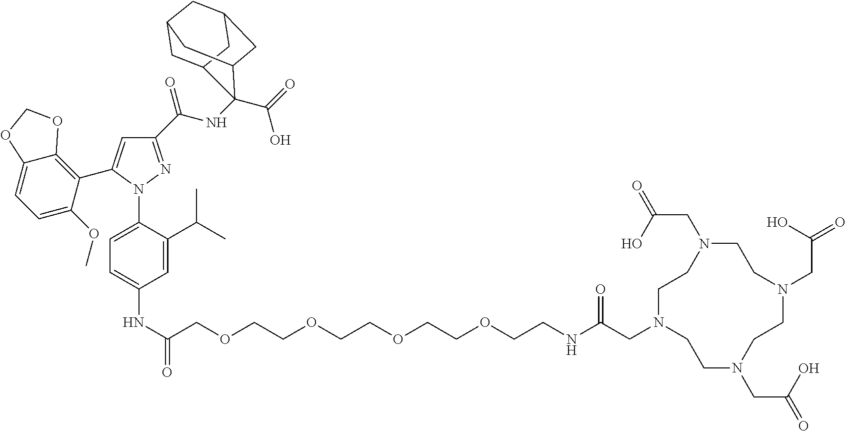 [SMILES: COc1ccc2c(c1-c1cc(C(=O)NC3(C(=O)O)C4CC5CC(C4)CC3C5)nn1-c1ccc(NC(=O)COCCOCCOCCOCCNC(=O)CN3CCN(CC(=O)O)CCN(CC(=O)O)CCN(CC(=O)O)CC3)cc1C(C)C)OCO2] | 1193 [M + H]⁺ |
| 16 | 2,2′,2″-(10-(2-((3-(4- (2-((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)amino)- 2- oxoethyl)piperidin-1- yl)propyl)amino)-2- oxoethyl)-1,4,7,10- tetraazacyclododecane- 1,4,7-triyl)triacetic acid | 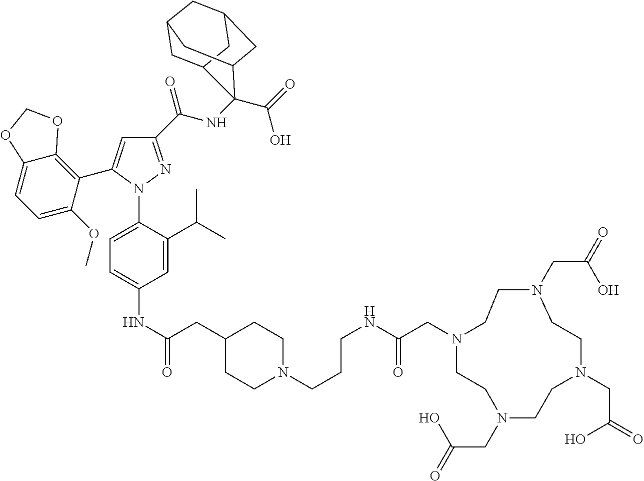 [SMILES: COc1ccc2c(c1-c1cc(C(=O)NC3(C(=O)O)C4CC5CC(C4)CC3C5)nn1-c1ccc(NC(=O)CC3CCN(CCCNC(=O)CN4CCN(CC(=O)O)CCN(CC(=O)O)CCN(CC(=O)O)CC4)CC3)cc1C(C)C)OCO2] | 1142 [M + H]⁺ |
| 17 | 2,2′,2″-(10-(3-(3-((3- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)(meth- yl)amino)propyl)(meth- yl)amino)propoxy)pro- pyl)-1,4,7,10- tetraazacyclododecane- 1,4,7-triyl)triacetic acid | 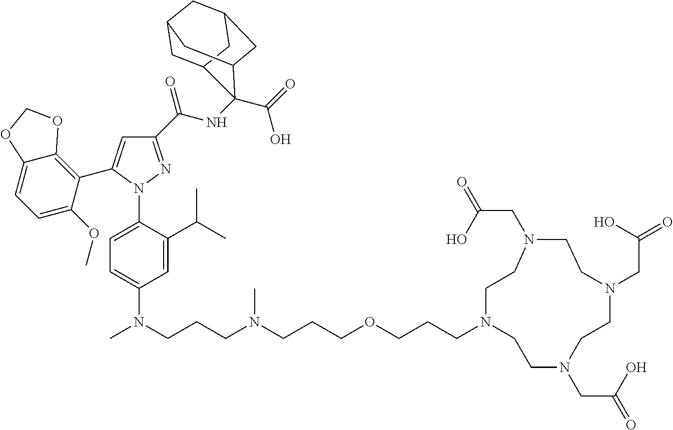 [SMILES: COc1ccc2c(c1-c1cc(C(=O)NC3(C(=O)O)C4CC5CC(C4)CC3C5)nn1-c1ccc(N(C)CCCN(C)CCCOCCCN3CCN(CC(=O)O)CCN(CC(=O)O)CCN(CC(=O)O)CC3)cc1C(C)C)OCO2] | 1103 [M + H]⁺ |
| 18 | 2,2′,2″-(10-(2-((2-((2- (4-(3-(((5r,7r)-2- carboxyadamantan-2- yl)carbamoyl)-5-(5- methoxybenzo[d][1,3] dioxol-4-yl)-1H- pyrazol-1-yl)-3- isopropylphenoxy)eth- yl)(methyl)amino)ethyl) amino)-2-oxoethyl)- 1,4,7,10- tetraazacyclododecane- 1,4,7-triyl)triacetic acid | 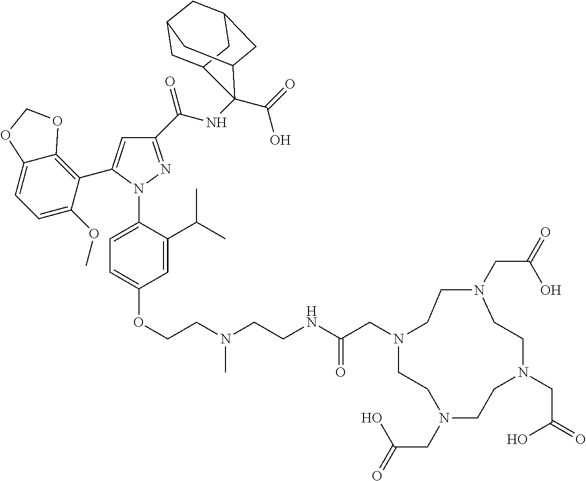 [SMILES: COc1ccc2c(c1-c1cc(C(=O)NC3(C(=O)O)C4CC5CC(C4)CC3C5)nn1-c1ccc(OCCN(C)CCNC(=O)CN3CCN(CC(=O)O)CCN(CC(=O)O)CCN(CC(=O)O)CC3)cc1C(C)C)OCO2] | 1061 [M + H]⁺ |
| 19 | 2,2′,2″-(10-(2-((2-((2- (((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)carba- moyl)oxy)ethyl)(meth- yl)amino)ethyl)amino)- 2-oxoethyl)-1,4,7,10- tetraazacyclododecane- 1,4,7-triyl)triacetic acid | 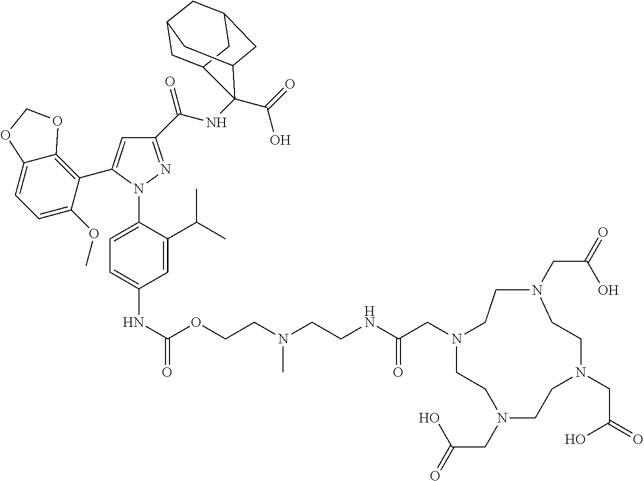 [SMILES: COc1ccc2c(c1-c1cc(C(=O)NC3(C(=O)O)C4CC5CC(C4)CC3C5)nn1-c1ccc(NC(=O)OCCN(C)CCNC(=O)CN3CCN(CC(=O)O)CCN(CC(=O)O)CCN(CC(=O)O)CC3)cc1C(C)C)OCO2] | 1104 [M + H]⁺ |
| 20 | 2,2′,2″-(10-(2-((3-((3- (3-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)ureido) propyl)(methyl)amino) propyl)amino)-2- oxoethyl)-1,4,7,10- tetraazacyclododecane- 1,4,7-triyl)triacetic acid | 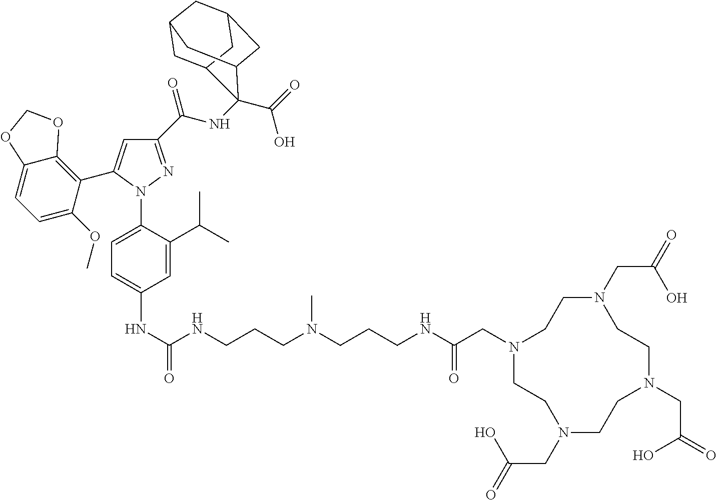 [SMILES: COc1ccc2c(c1-c1cc(C(=O)NC3(C(=O)O)C4CC5CC(C4)CC3C5)nn1-c1ccc(NC(=O)NCCCN(C)CCCNC(=O)CN3CCN(CC(=O)O)CCN(CC(=O)O)CCN(CC(=O)O)CC3)cc1C(C)C)OCO2] | 1131 [M + H]⁺ |
| 21 | 2,2′,2″-(10-(2-((3-((3- (5-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)picoli- namido)propyl)(methyl) amino)propyl)amino)- 2-oxoethyl)-1,4,7,10- tetraazacyclododecane- 1,4,7-triyl)triacetic acid | 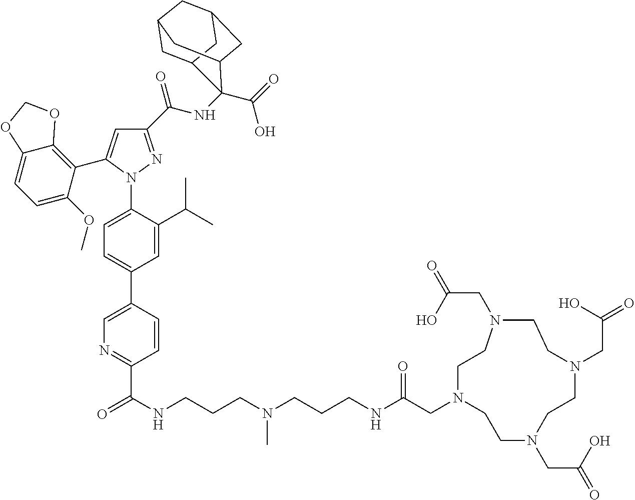 [SMILES: COc1ccc2c(c1-c1cc(C(=O)NC3(C(=O)O)C4CC5CC(C4)CC3C5)nn1-c1ccc(-c3ccc(C(=O)NCCCN(C)CCCNC(=O)CN4CCN(CC(=O)O)CCN(CC(=O)O)CCN(CC(=O)O)CC4)nc3)cc1C(C)C)OCO2] | 1193 [M + H]⁺ |
| 22 | 2,2′,2″-(10-(2-((3-((3- (4-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-1H- 1,2,3-triazol-1- yl)propyl)(methyl)ami- no)propyl)amino)-2- oxoethyl)-1,4,7,10- tetraazacyclododecane- 1,4,7-triyl)triacetic acid | 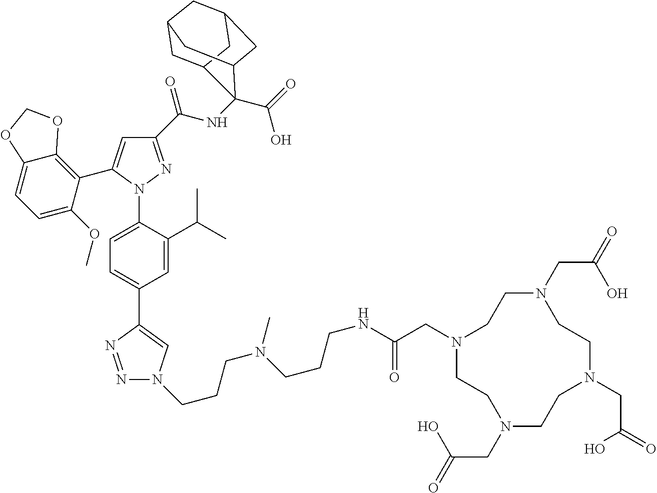 [SMILES: COc1ccc2c(c1-c1cc(C(=O)NC3(C(=O)O)C4CC5CC(C4)CC3C5)nn1-c1ccc(-c3cn(CCCN(C)CCCNC(=O)CN4CCN(CC(=O)O)CCN(CC(=O)O)CCN(CC(=O)O)CC4)nn3)cc1C(C)C)OCO2] | 1140 [M + H]⁺ |
| 23 | 2,2′,2″-(10-(2-((3-(3- (4-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-1H- 1,2,3-triazol-1- yl)propoxy)propyl)ami- no)-2-oxoethyl)- 1,4,7,10- tetraazacyclododecane- 1,4,7-triyl)triacetic acid | 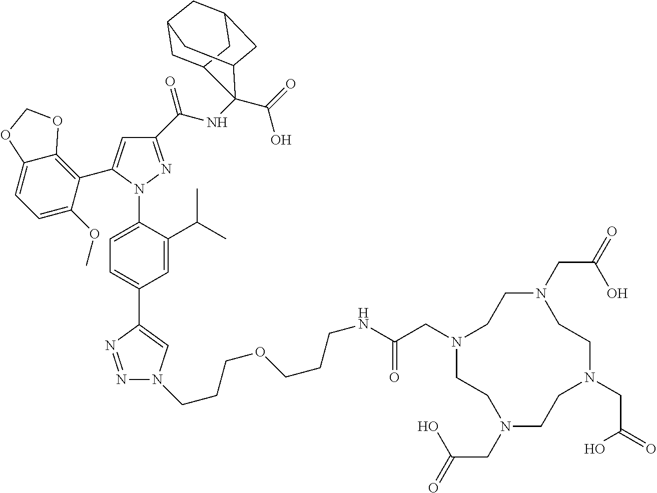 [SMILES: COc1ccc2c(c1-c1cc(C(=O)NC3(C(=O)O)C4CC5CC(C4)CC3C5)nn1-c1ccc(-c3cn(CCCOCCCNC(=O)CN4CCN(CC(=O)O)CCN(CC(=O)O)CCN(CC(=O)O)CC4)nn3)cc1C(C)C)OCO2] | 1127 [M + H]⁺ |
| 24 | 2,2′,2″-(10-(1- Carboxy-4-((3-((3-(3- (4-(3-(((5r,7r)-2- carboxyadamantan-2- yl)carbamoyl)-5-(5- methoxybenzo[d][1,3] dioxol-4-yl)-1H- pyrazol-1-yl)-3- isopropylphenyl)-1- methylureido)propyl)(meth- yl)amino)propyl)(meth- yl)amino)-4- oxobutyl)-1,4,7,10- tetraazacyclododecane- 1,4,7-triyl)triacetic acid | 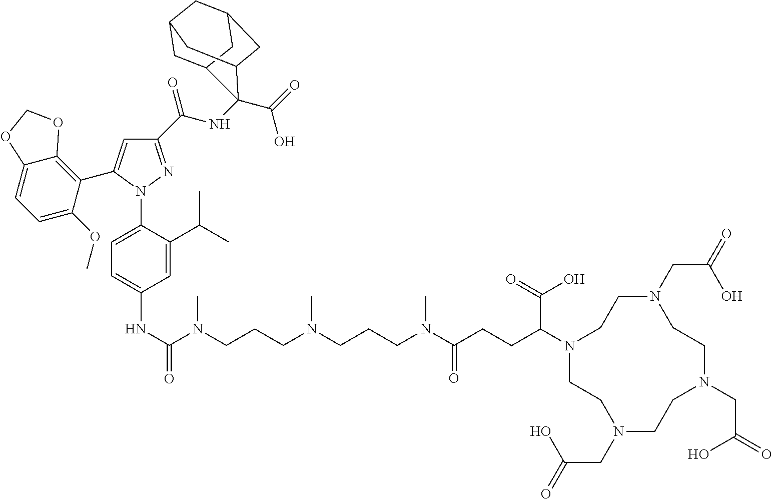 [SMILES: COc1ccc2c(c1-c1cc(C(=O)NC3(C(=O)O)C4CC5CC(C4)CC3C5)nn1-c1ccc(NC(=O)N(C)CCCN(C)CCCN(C)C(=O)CCC(C(=O)O)N3CCN(CC(=O)O)CCN(CC(=O)O)CCN(CC(=O)O)CC3)cc1C(C)C)OCO2] | 1231 [M + H]⁺ |
| 25 | 2,2′,2″-(10-(2-((3-((3- (4-(5-(Benzo[1,2- d:4,5- d′]bis([1,3]dioxole)-4- yl)-3-(((5r,7r)-2- carboxyadamantan-2- yl)carbamoyl)-1H- pyrazol-1-yl)-3- isopropyl-N- methylbenzamido)pro- pyl)(methyl)amino)pro- pyl)(methyl)amino)-2- oxoethyl)-1,4,7,10- tetraazacyclododecane- 1,4,7-triyl)triacetic acid | 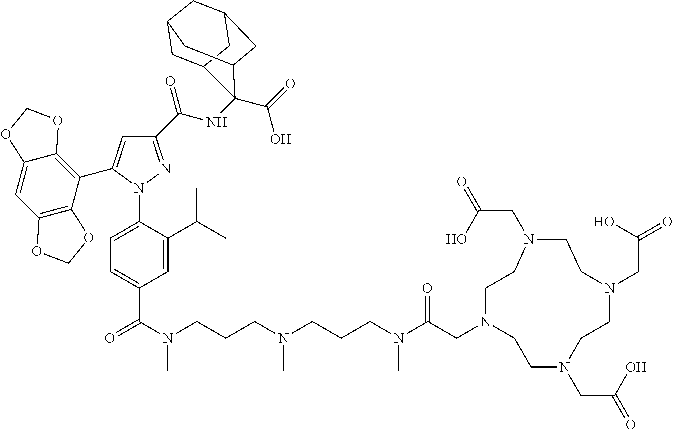 [SMILES: CC(C)c1cc(C(=O)N(C)CCCN(C)CCCN(C)C(=O)CN2CCN(CC(=O)O)CCN(CC(=O)O)CCN(CC(=O)O)CC2)ccc1-n1nc(C(=O)NC2(C(=O)O)C3CC4CC(C3)CC2C4)cc1-c1c2c(cc3c1OCO3)OCO2] | 1158 [M + H]⁺ |
| 26 | 2,2′,2″-(10-(2-((3-((3- (3-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)quino- line-7- carboxamido)propyl)(meth- yl)amino)propyl) amino)-2-oxoethyl)- 1,4,7,10- tetraazacyclododecane- 1,4,7-triyl)triacetic acid | 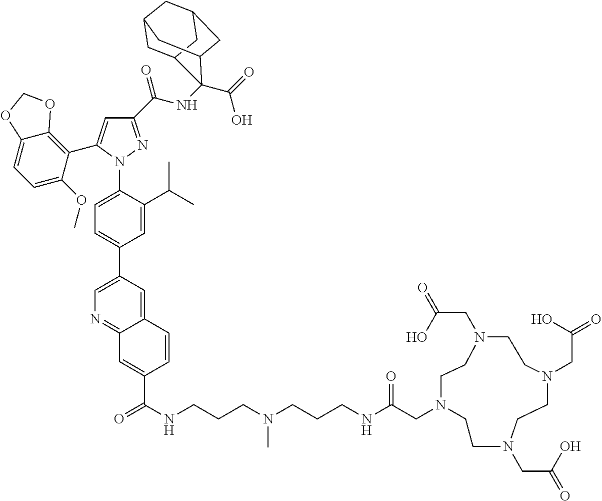 [SMILES: COc1ccc2c(c1-c1cc(C(=O)NC3(C(=O)O)C4CC5CC(C4)CC3C5)nn1-c1ccc(-c3cnc4cc(C(=O)NCCCN(C)CCCNC(=O)CN5CCN(CC(=O)O)CCN(CC(=O)O)CCN(CC(=O)O)CC5)ccc4c3)cc1C(C)C)OCO2] | 1243 [M + H]⁺ |
| 35 | 2,2′,2″-(10-(2-((3- ((1R,4R)-5-(4-(3- (((5R,7R)-2- Carboxyadamantan-2- yl)carbamoyl)-5-(5- methoxybenzo[d][1,3] dioxol-4-yl)-1H- pyrazol-1-yl)-3- isopropylphenyl)-2,5- diazabicyclo[2.2.1]hep- tan-2- yl)propyl)amino)-2- oxoethyl)-1,4,7,10- tetraazacyclododecane- 1,4,7-triyl)triacetic acid | 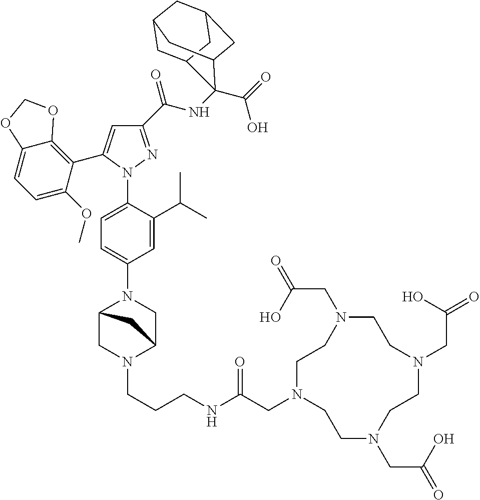 [SMILES: COc1ccc2c(c1-c1cc(C(=O)NC3(C(=O)O)C4CC5CC(C4)CC3C5)nn1-c1ccc(N3C[C@H]4C[C@@H]3CN4CCCNC(=O)CN3CCN(CC(=O)O)CCN(CC(=O)O)CCN(CC(=O)O)CC3)cc1C(C)C)OCO2] | 1098 [M + H]⁺ |
| 36 | 2,2′,2″-(10-(2-((3- ((1S,4S)-5-(4-(3- (((5S,7S)-2- Carboxyadamantan-2- yl)carbamoyl)-5-(5- methoxybenzo[d][1,3] dioxol-4-yl)-1H- pyrazol-1-yl)-3- isopropylphenyl)-2,5- diazabicyclo[2.2.1]hep- tan-2- yl)propyl)amino)-2- oxoethyl)-1,4,7,10- tetraazacyclododecane- 1,4,7-triyl)triacetic acid | 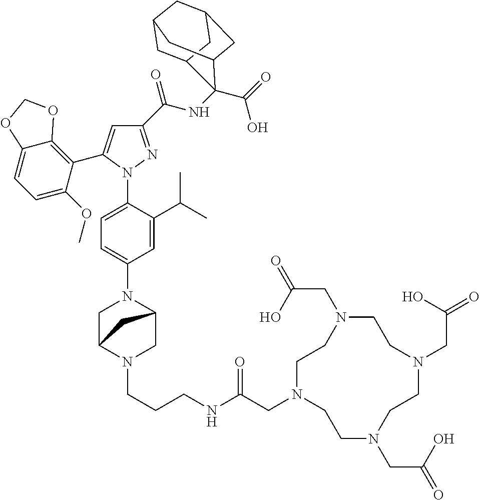 [SMILES: COc1ccc2c(c1-c1cc(C(=O)NC3(C(=O)O)C4CC5CC(C4)CC3C5)nn1-c1ccc(N3C[C@@H]4C[C@H]3CN4CCCNC(=O)CN3CCN(CC(=O)O)CCN(CC(=O)O)CCN(CC(=O)O)CC3)cc1C(C)C)OCO2] | 1098 [M + H]⁺ |
| 37 | 2,2′,2″-(10-(2-((3-((3- (6-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)quin- oxaline-2- carboxamido)propyl)(meth- yl)amino)propyl) amino)-2-oxoethyl)- 1,4,7,10- tetraazacyclododecane- 1,4,7-triyl)triacetic acid | 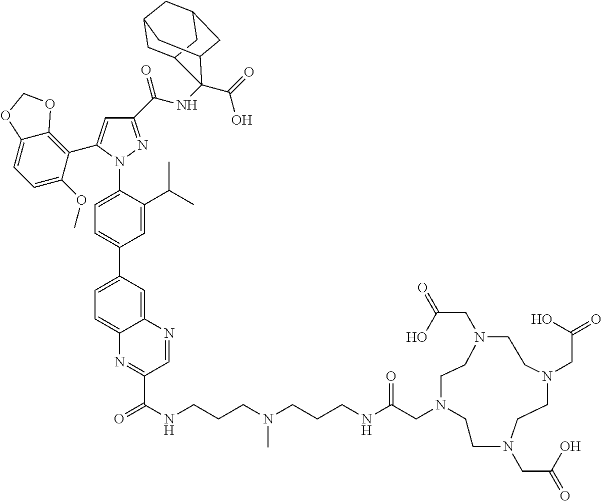 [SMILES: COc1ccc2c(c1-c1cc(C(=O)NC3(C(=O)O)C4CC5CC(C4)CC3C5)nn1-c1ccc(-c3ccc4nc(C(=O)NCCCN(C)CCCNC(=O)CN5CCN(CC(=O)O)CCN(CC(=O)O)CCN(CC(=O)O)CC5)cnc4c3)cc1C(C)C)OCO2] | 1244 [M + H]⁺ |
| 38 | 2,2′,2″-(10-(2-((3-((3- (7-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)quino- line-3- carboxamido)propyl)(meth- yl)amino)propyl) amino)-2-oxoethyl)- 1,4,7,10- tetraazacyclododecane- 1,4,7-triyl)triacetic acid | 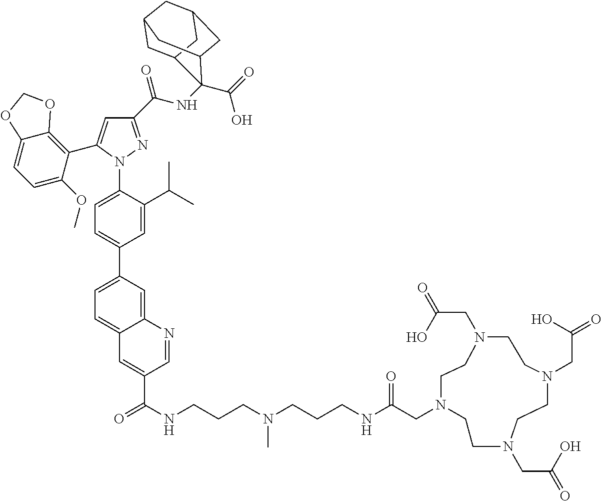 [SMILES: COc1ccc2c(c1-c1cc(C(=O)NC3(C(=O)O)C4CC5CC(C4)CC3C5)nn1-c1ccc(-c3ccc4cc(C(=O)NCCCN(C)CCCNC(=O)CN5CCN(CC(=O)O)CCN(CC(=O)O)CCN(CC(=O)O)CC5)cnc4c3)cc1C(C)C)OCO2] | 1243 [M + H]⁺ |
| 39 | 2,2′,2″-(10-(2-((3-(7- (4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-2,7- diazaspiro[3.5]nonan- 2-yl)propyl)amino)-2- oxoethyl)-1,4,7,10- tetraazacyclododecane- 1,4,7-triyl)triacetic acid | 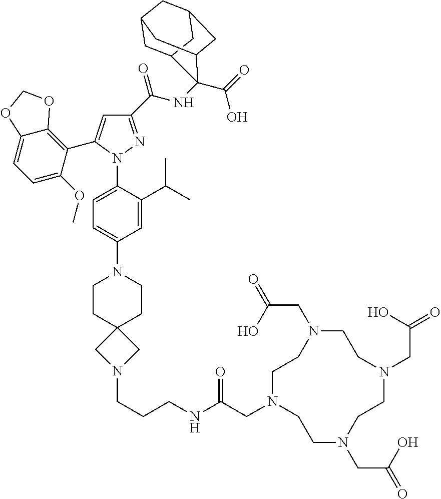 [SMILES: COc1ccc2c(c1-c1cc(C(=O)NC3(C(=O)O)C4CC5CC(C4)CC3C5)nn1-c1ccc(N3CCC4(CC3)CN(CCCNC(=O)CN3CCN(CC(=O)O)CCN(CC(=O)O)CCN(CC(=O)O)CC3)C4)cc1C(C)C)OCO2] | 1126 [M + H]⁺ |
| 40 | 2,2′,2″-(10-(2-((3-(2- (4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-2,7- diazaspiro[3.5]nonan- 7-yl)propyl)amino)-2- oxoethyl)-1,4,7,10- tetraazacyclododecane- 1,4,7-triyl)triacetic acid | 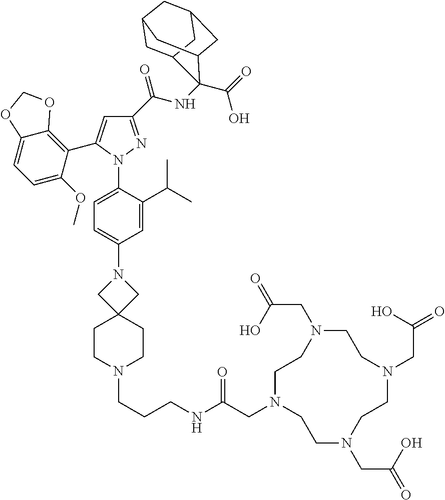 [SMILES: COc1ccc2c(c1-c1cc(C(=O)NC3(C(=O)O)C4CC5CC(C4)CC3C5)nn1-c1ccc(N3CC4(CCN(CCCNC(=O)CN5CCN(CC(=O)O)CCN(CC(=O)O)CCN(CC(=O)O)CC5)CC4)C3)cc1C(C)C)OCO2] | 1126 [M + H]⁺ |
| 41 | 2,2′,2″-(10-(2-((3-((3- (4-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)piper- azin-1- yl)propyl)(methyl)ami- no)propyl)amino)-2- oxoethyl)-1,4,7,10- tetraazacyclododecane- 1,4,7-triyl)triacetic acid | 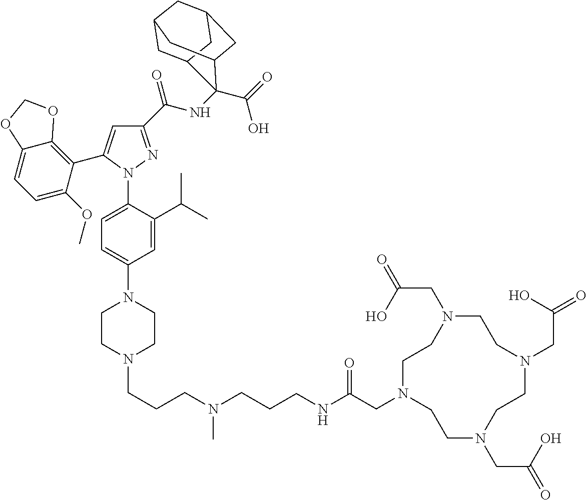 [SMILES: COc1ccc2c(c1-c1cc(C(=O)NC3(C(=O)O)C4CC5CC(C4)CC3C5)nn1-c1ccc(N3CCN(CCCN(C)CCCNC(=O)CN4CCN(CC(=O)O)CCN(CC(=O)O)CCN(CC(=O)O)CC4)CC3)cc1C(C)C)OCO2] | 1157 [M + H]⁺ |
| 42 | 2,2′,2″-(4-(2-((3-((3- ((3-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)quino- lin-7- yl)oxy)propyl)(methyl) amino)propyl)amino)- 2-oxoethyl)-1,4,7- triazacyclododecane- 1,7,10-triyl)triacetic acid | 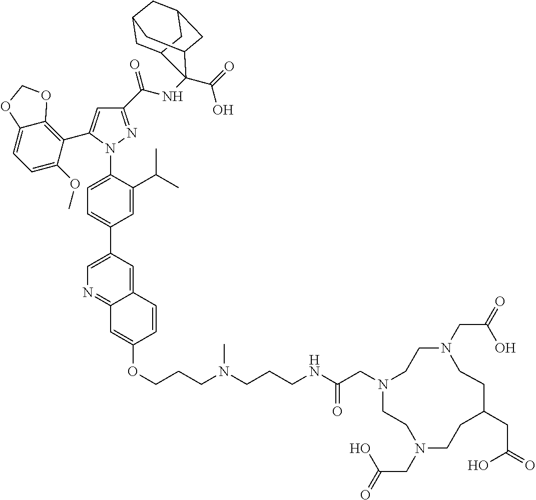 [SMILES: COc1ccc2c(c1-c1cc(C(=O)NC3(C(=O)O)C4CC5CC(C4)CC3C5)nn1-c1ccc(-c3cnc4cc(OCCCN(C)CCCNC(=O)CN5CCN(CC(=O)O)CCC(CC(=O)O)CCN(CC(=O)O)CC5)ccc4c3)cc1C(C)C)OCO2] | 1216 [M + H]⁺ |
| 43 | 2,2′,2″-(10-(2-((3-((3- (7-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-1,5- naphthyridine-3- carboxamido)propyl)(meth- yl)amino)propyl) amino)-2-oxoethyl)- 1,4,7,10- tetraazacyclododecane- 1,4,7-triyl)triacetic acid | 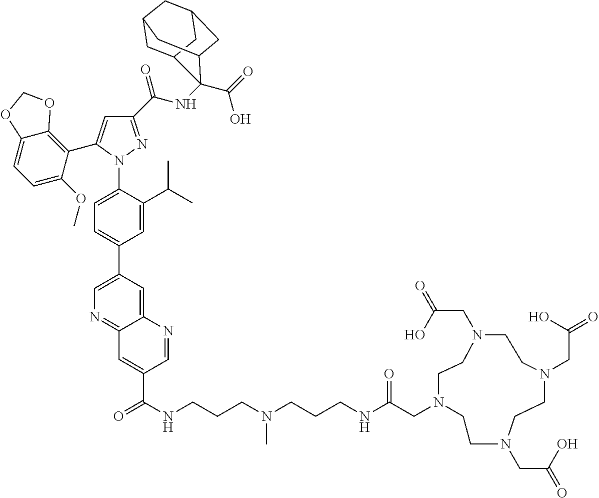 [SMILES: COc1ccc2c(c1-c1cc(C(=O)NC3(C(=O)O)C4CC5CC(C4)CC3C5)nn1-c1ccc(-c3cnc4cc(C(=O)NCCCN(C)CCCNC(=O)CN5CCN(CC(=O)O)CCN(CC(=O)O)CCN(CC(=O)O)CC5)cnc4c3)cc1C(C)C)OCO2] | 1244 [M + H]⁺ |
| 44 | 2,2′,2″-(10-(2-((3-((3- (5-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-3- fluoropicolinamido)pro- pyl)(methyl)amino)pro- pyl)amino)-2- oxoethyl)-1,4,7,10- tetraazacyclododecane- 1,4,7-triyl)triacetic acid | 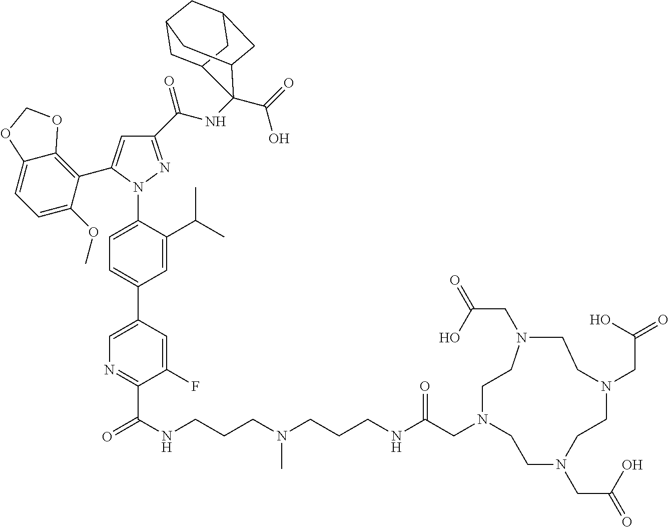 [SMILES: COc1ccc2c(c1-c1cc(C(=O)NC3(C(=O)O)C4CC5CC(C4)CC3C5)nn1-c1ccc(-c3cnc(C(=O)NCCCN(C)CCCNC(=O)CN4CCN(CC(=O)O)CCN(CC(=O)O)CCN(CC(=O)O)CC4)c(F)c3)cc1C(C)C)OCO2] | 1211 [M + H]⁺ |
| 45 | 2,2′,2″-(10-(1-((4-(3- (((5r,7r)-2- Carboxyadamantan-2- yl)carbamoyl)-5-(5- methoxybenzo[d][1,3] dioxol-4-yl)-1H- pyrazol-1-yl)-3- isopropylphenyl)amino)- 13-methyl-1,18- dioxo-3,6-dioxa- 9,13,17- triazanonadecan-19- yl)-1,4,7,10- tetraazacyclododecane- 1,4,7-triyl)triacetic acid | 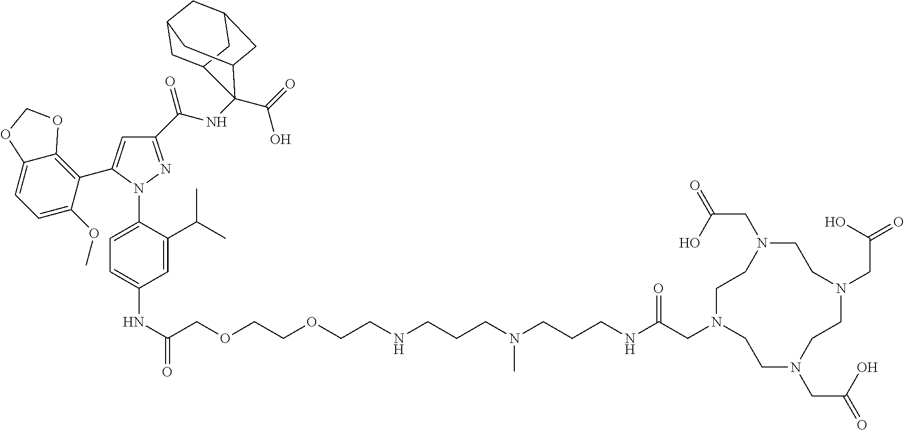 [SMILES: COc1ccc2c(c1-c1cc(C(=O)NC3(C(=O)O)C4CC5CC(C4)CC3C5)nn1-c1ccc(NC(=O)COCCOCCNCCCN(C)CCCNC(=O)CN3CCN(CC(=O)O)CCN(CC(=O)O)CCN(CC(=O)O)CC3)cc1C(C)C)OCO2] | 1234 [M + H]⁺ |
| 46 | 2,2′,2″-(10-(2-((3-(9- (4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-3,9- diazaspiro[5.5]undecan- 3-yl)propyl)amino)- 2-oxoethyl)-1,4,7,10- tetraazacyclododecane- 1,4,7-triyl)triacetic acid | 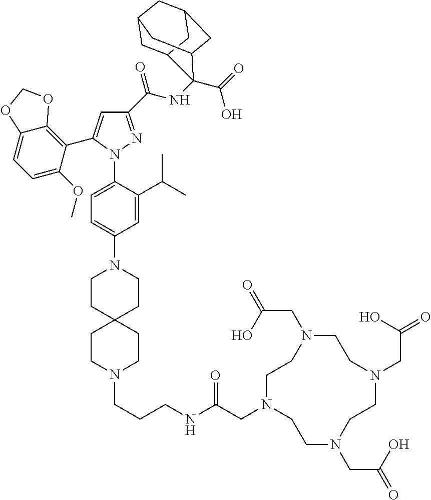 [SMILES: COc1ccc2c(c1-c1cc(C(=O)NC3(C(=O)O)C4CC5CC(C4)CC3C5)nn1-c1ccc(N3CCC4(CCN(CCCNC(=O)CN5CCN(CC(=O)O)CCN(CC(=O)O)CCN(CC(=O)O)CC5)CC4)CC3)cc1C(C)C)OCO2] | 1154 [M + H]⁺ |
| 47 | 2,2′,2″-(10-(2-(4-((1- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)carba- moyl)piperidin-4- yl)methyl)piperazin-1- yl)-2-oxoethyl)- 1,4,7,10- tetraazacyclododecane- 1,4,7-triyl)triacetic acid | 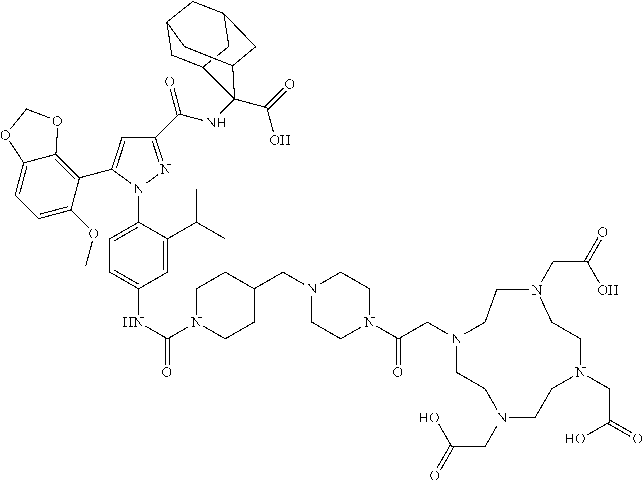 [SMILES: COc1ccc2c(c1-c1cc(C(=O)NC3(C(=O)O)C4CC5CC(C4)CC3C5)nn1-c1ccc(NC(=O)N3CCC(CN4CCN(C(=O)CN5CCN(CC(=O)O)CCN(CC(=O)O)CCN(CC(=O)O)CC5)CC4)CC3)cc1C(C)C)OCO2] | 1169 [M + H]⁺ |
| 48 | 2,2′,2″-(10-(2-((3-((3- (4′-(3-(((5r,7r)-2- Carboxyadamantan-2- yl)carbamoyl)-5-(5- methoxybenzo[d][1,3] dioxol-4-yl)-1H- pyrazol-1-yl)-2,6- difluoro-3′-isopropyl- [1,1′-biphenyl]-4- carboxamido)propyl)(meth- yl)amino)propyl) amino)-2-oxoethyl)- 1,4,7,10- tetraazacyclododecane- 1,4,7-triyl)triacetic acid | 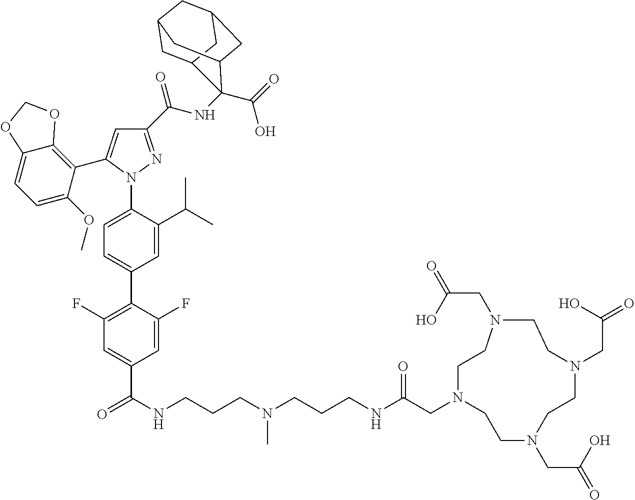 [SMILES: COc1ccc2c(c1-c1cc(C(=O)NC3(C(=O)O)C4CC5CC(C4)CC3C5)nn1-c1ccc(-c3c(F)cc(C(=O)NCCCN(C)CCCNC(=O)CN4CCN(CC(=O)O)CCN(CC(=O)O)CCN(CC(=O)O)CC4)cc3F)cc1C(C)C)OCO2] | 1228 [M + H]⁺ |
| 49 | 2,2′,2″-(10-(2-((3-((3- (4′-(3-(((5r,7r)-2- Carboxyadamantan-2- yl)carbamoyl)-5-(5- methoxybenzo[d][1,3] dioxol-4-yl)-1H- pyrazol-1-yl)-3′- isopropyl-3-methoxy- [1,1′-biphenyl]-4- carboxamido)propyl)(meth- yl)amino)propyl) amino)-2-oxoethyl)- 1,4,7,10- tetraazacyclododecane- 1,4,7-triyl)triacetic acid | 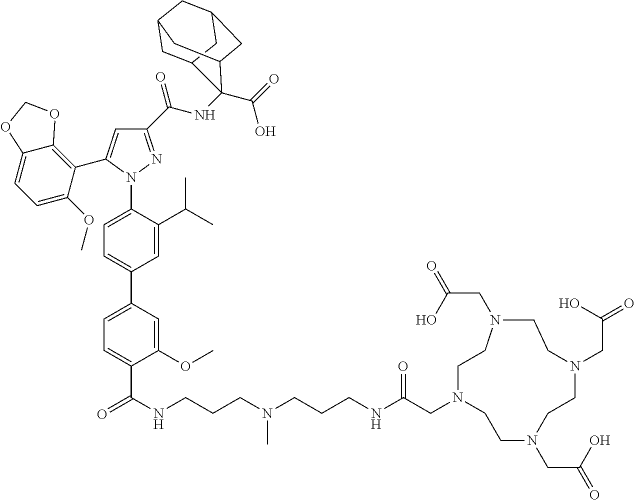 [SMILES: COc1cc(-c2ccc(-n3nc(C(=O)NC4(C(=O)O)C5CC6CC(C5)CC4C6)cc3-c3c(OC)ccc4c3OCO4)c(C(C)C)c2)ccc1C(=O)NCCCN(C)CCCNC(=O)CN1CCN(CC(=O)O)CCN(CC(=O)O)CCN(CC(=O)O)CC1] | 1222 [M + H]⁺ |
| 50 | 2,2′,2″-(10-(2-((3-(4- (3-((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)amino) propyl)piperazin-1- yl)propyl)amino)-2- oxoethyl)-1,4,7,10- tetraazacyclododecane- 1,4,7-triyl)triacetic acid | 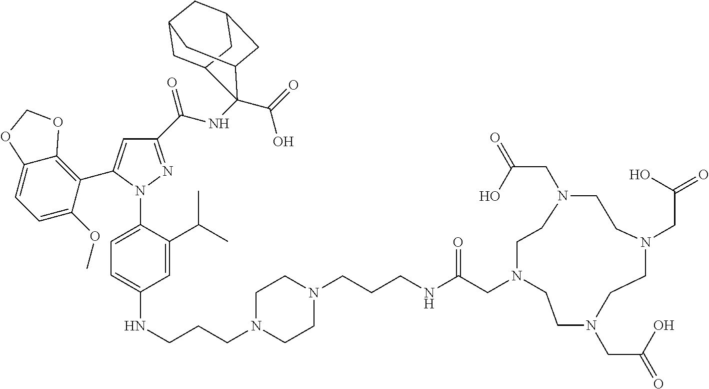 [SMILES: COc1ccc2c(c1-c1cc(C(=O)NC3(C(=O)O)C4CC5CC(C4)CC3C5)nn1-c1ccc(NCCCN3CCN(CCCNC(=O)CN4CCN(CC(=O)O)CCN(CC(=O)O)CCN(CC(=O)O)CC4)CC3)cc1C(C)C)OCO2] | 1143 [M + H]⁺ |
| 51 | 2,2′,2″-(10-(2-(4-(2-(4- (3-(((5r,7r)-2- Carboxyadamantan-2- yl)carbamoyl)-5-(5- methoxybenzo[d][1,3] dioxol-4-yl)-1H- pyrazol-1-yl)-3- isopropylphenoxy)eth- yl)piperazin-1-yl)-2- oxoethyl)-1,4,7,10- tetraazacyclododecane- 1,4,7-triyl)triacetic acid | 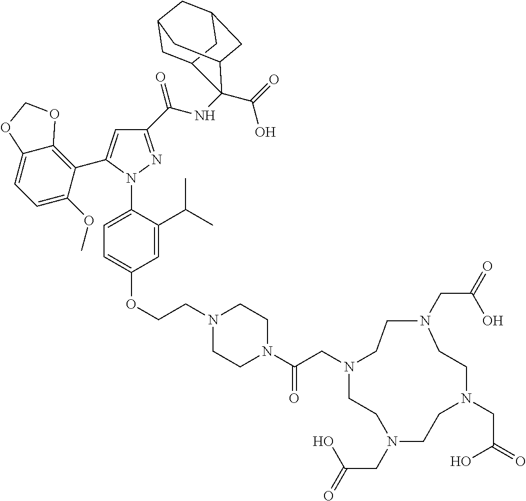 [SMILES: COc1ccc2c(c1-c1cc(C(=O)NC3(C(=O)O)C4CC5CC(C4)CC3C5)nn1-c1ccc(OCCN3CCN(C(=O)CN4CCN(CC(=O)O)CCN(CC(=O)O)CCN(CC(=O)O)CC4)CC3)cc1C(C)C)OCO2] | 1073 [M + H]⁺ |
| 52 | 2,2′,2″-(10-(2-((3-((4- ((3-((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)amino) propyl)(methyl)amino) butyl)(methyl)amino) propyl)amino)-2- oxoethyl)-1,4,7,10- tetraazacyclododecane- 1,4,7-triyl)triacetic acid | 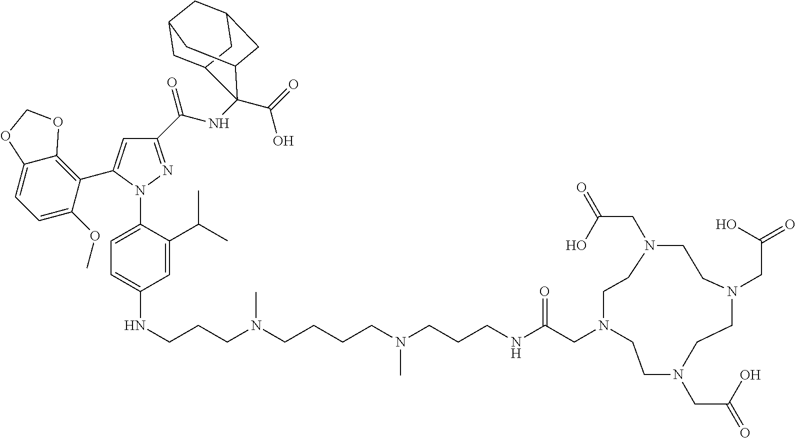 [SMILES: COc1ccc2c(c1-c1cc(C(=O)NC3(C(=O)O)C4CC5CC(C4)CC3C5)nn1-c1ccc(NCCCN(C)CCCCN(C)CCCNC(=O)CN3CCN(CC(=O)O)CCN(CC(=O)O)CCN(CC(=O)O)CC3)cc1C(C)C)OCO2] | 1173 [M + H]⁺ |
| 54 | 2,2′,2″-(10-(2-(((2S)- 4-Carboxy-1-(4-(2-(4- (3-(((5r,7r)-2- carboxyadamantan-2- yl)carbamoyl)-5-(5- methoxybenzo[d][1,3] dioxol-4-yl)-1H- pyrazol-1-yl)-3- isopropylphenoxy)eth- yl)piperidin-1-yl)-1- oxobutan-2-yl)amino)- 2-oxoethyl)-1,4,7,10- tetraazacyclododecane- 1,4,7-triyl)triacetic acid | 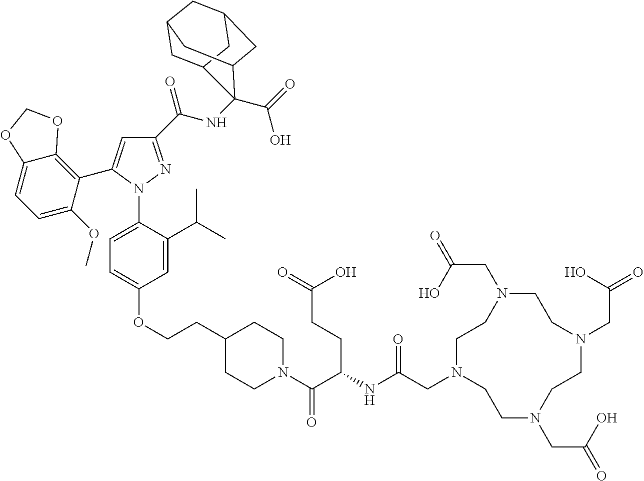 [SMILES: COc1ccc2c(c1-c1cc(C(=O)NC3(C(=O)O)C4CC5CC(C4)CC3C5)nn1-c1ccc(OCCC3CCN(C(=O)[C@H](CCC(=O)O)NC(=O)CN4CCN(CC(=O)O)CCN(CC(=O)O)CCN(CC(=O)O)CC4)CC3)cc1C(C)C)OCO2] | 1201 [M + H]⁺ |
| 55 | 2,2′,2″-(10-(2-(4-(3-(4- (3-(((5r,7r)-2- Carboxyadamantan-2- yl)carbamoyl)-5-(5- methoxybenzo[d][1,3] dioxol-4-yl)-1H- pyrazol-1-yl)-3- isopropylphenoxy)pro- pyl)piperazin-1-yl)-2- oxoethyl)-1,4,7,10- tetraazacyclododecane- 1,4,7-triyl)triacetic acid | 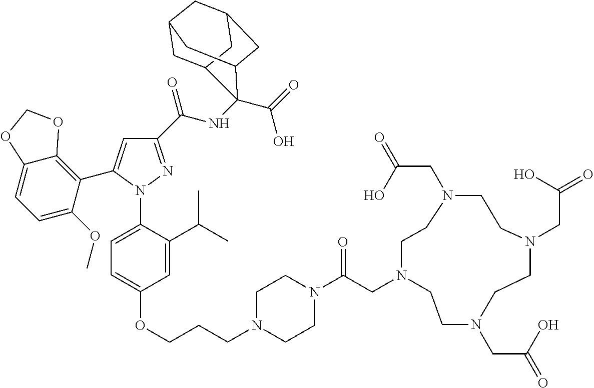 [SMILES: COc1ccc2c(c1-c1cc(C(=O)NC3(C(=O)O)C4CC5CC(C4)CC3C5)nn1-c1ccc(OCCCN3CCN(C(=O)CN4CCN(CC(=O)O)CCN(CC(=O)O)CCN(CC(=O)O)CC4)CC3)cc1C(C)C)OCO2] | 1087 [M + H]⁺ |
| 56 | 2,2′,2″-(10-(2-((2-((1- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)carba- moyl)piperidin-4- yl)oxy)ethyl)amino)- 2-oxoethyl)-1,4,7,10- tetraazacyclododecane- 1,4,7-triyl)triacetic acid | 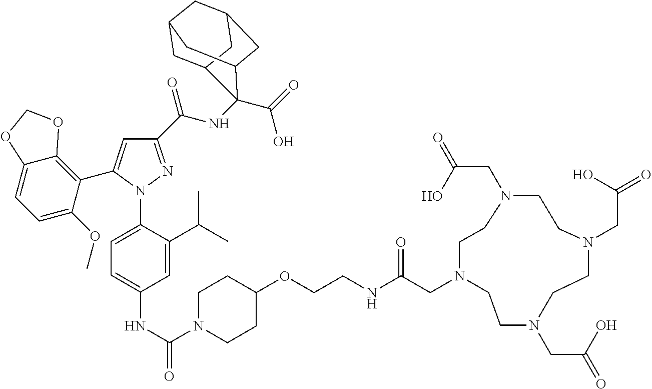 [SMILES: COc1ccc2c(c1-c1cc(C(=O)NC3(C(=O)O)C4CC5CC(C4)CC3C5)nn1-c1ccc(NC(=O)N3CCC(OCCNC(=O)CN4CCN(CC(=O)O)CCN(CC(=O)O)CCN(CC(=O)O)CC4)CC3)cc1C(C)C)OCO2] | 1130 [M + H]⁺ |
| 57 | 2,2′,2″-(10-(2-((3-((3- ((4′-(3-(((5r,7r)-2- Carboxyadamantan-2- yl)carbamoyl)-5-(5- methoxybenzo[d][1,3] dioxol-4-yl)-1H- pyrazol-1-yl)-3′- isopropyl-[1,1′- biphenyl]-4- yl)oxy)propyl)(methyl) amino)propyl)amino)- 2-oxoethyl)-1,4,7,10- tetraazacyclododecane- 1,4,7-triyl)triacetic acid | 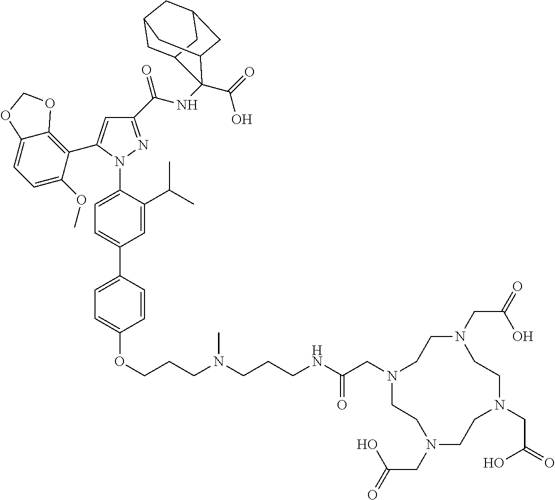 [SMILES: COc1ccc2c(c1-c1cc(C(=O)NC3(C(=O)O)C4CC5CC(C4)CC3C5)nn1-c1ccc(-c3ccc(OCCCN(C)CCCNC(=O)CN4CCN(CC(=O)O)CCN(CC(=O)O)CCN(CC(=O)O)CC4)cc3)cc1C(C)C)OCO2] | 1165 [M + H]⁺ |
| 58 | 2,2′,2″-(10-(2-((3-(9- (4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-8- oxo-3,9- diazaspiro[5.5]undecan- 3-yl)propyl)amino)- 2-oxoethyl)-1,4,7,10- tetraazacyclododecane- 1,4,7-triyl)triacetic acid | 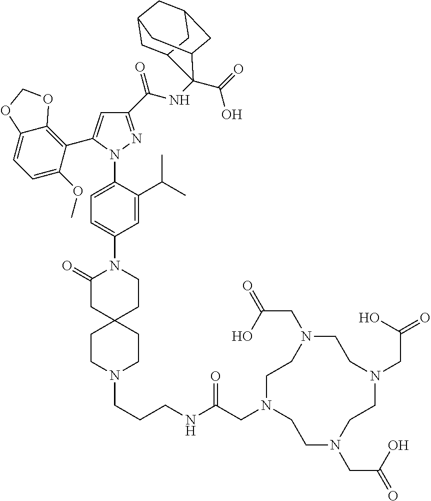 [SMILES: COc1ccc2c(c1-c1cc(C(=O)NC3(C(=O)O)C4CC5CC(C4)CC3C5)nn1-c1ccc(N3CCC4(CCN(CCCNC(=O)CN5CCN(CC(=O)O)CCN(CC(=O)O)CCN(CC(=O)O)CC5)CC4)CC3=O)cc1C(C)C)OCO2] | 1168 [M + H]⁺ |
| 59 | 2,2′,2″-(10-(1- Carboxy-4-((3-((3-(3- (4-(3-(((5r,7r)-2- carboxyadamantan-2- yl)carbamoyl)-5-(5- methoxybenzo[d][1,3] dioxol-4-yl)-1H- pyrazol-1-yl)-3- isopropylphenyl)ureido) propyl)(methyl)amino) propyl)amino)-4- oxobutyl)-1,4,7,10- tetraazacyclododecane- 1,4,7-triyl)triacetic acid | 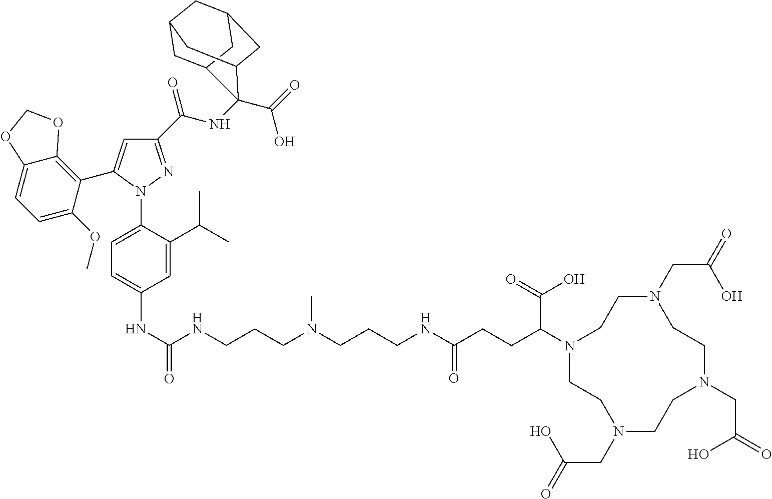 [SMILES: COc1ccc2c(c1-c1cc(C(=O)NC3(C(=O)O)C4CC5CC(C4)CC3C5)nn1-c1ccc(NC(=O)NCCCN(C)CCCNC(=O)CCC(C(=O)O)N3CCN(CC(=O)O)CCN(CC(=O)O)CCN(CC(=O)O)CC3)cc1C(C)C)OCO2] | 1203 [M + H]⁺ |

### Example 27

### 2,2′,2″-(10-(2-((3-((4-((4-(3-(((5r,7r)-2-Carboxyadamantan-2-yl)carbamoyl)-5-(5-methoxybenzo[d][1,3]dioxol-4-yl)-1H-pyrazol-1-yl)-3-isopropylphenyl)amino)-4-oxobutyl)(3-(dimethylamino)propyl)amino)propyl)(methyl)amino)-2-oxoethyl)-1,4,7,10-tetraazacyclododecane-1,4,7-triyl)triacetic acid

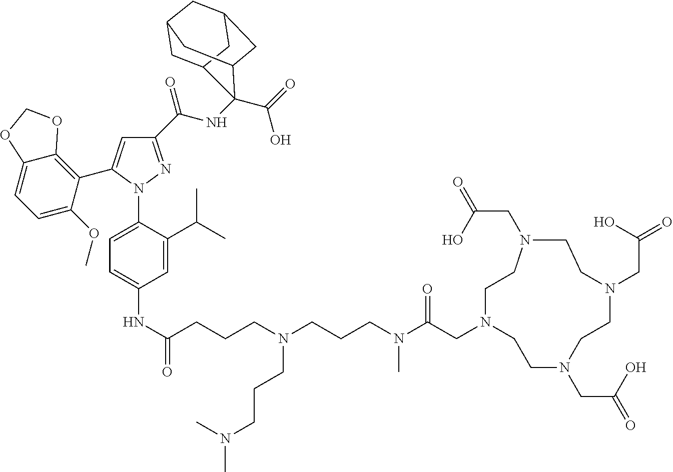 [SMILES: COc1ccc2c(c1-c1cc(C(=O)NC3(C(=O)O)C4CC5CC(C4)CC3C5)nn1-c1ccc(NC(=O)CCCN(CCCN(C)C)CCCN(C)C(=O)CN3CCN(CC(=O)O)CCN(CC(=O)O)CCN(CC(=O)O)CC3)cc1C(C)C)OCO2]

A soln of tert-butyl 2-{1-[4-(4-{[3-(dimethylamino)propyl][3-(N-methyl-2-{4,7,10-tris[2-(tert-butoxy)-2-oxoethyl]-1,4,7,10-tetraazacyclododecan-1-yl}acetamido)propyl]amino}butanamido)-2-isopropylphenyl]-5-(5-methoxy-2H-1,3-benzodioxol-4-yl)pyrazole-3-amido}adamantane-2-carboxylate (61 mg, 0.043 mmol) in DCM (2 mL) at RT under N₂ was treated with HCl in 1,4-dioxane (4.0 M, 2 mL). After stirring for 1 h, the mixture was concentrated and purified by Prep-HPLC with the following conditions: Column, Xselect CSH C18 OBD 30*150 mm, 5 μm; Mobile Phase, 16 to 26% ACN in H₂O (0.1% FA) to afford title compound (41 mg, 79.7%) as a white solid. MS ES+ m/z 1201 [M+H]⁺.

The following compounds were prepared in a manner essentially analogous to the preparation method of 2,2′,2″-(10-(2-((3-((4-((4-(3-(((5r,7r)-2-Carboxyadamantan-2-yl)carbamoyl)-5-(5-methoxybenzo[d][1,3]dioxol-4-yl)-1H-pyrazol-1-yl)-3-isopropylphenyl)amino)-4-oxobutyl)(3-(dimethylamino)propyl)amino)propyl)(methyl)amino)-2-oxoethyl)-1,4,7,10-tetraazacyclododecane-1,4,7-triyl)triacetic acid using the appropriate reagents, adjusting temperature, adjusting reaction time to determine completion of the reaction, and adjusting the purification system as appropriate.

TABLE 45

| | | | MS |
|---|---|---|---|
| | | | ES+ |
| Ex # | Chemical Name | Structure | m/z |
| 28 | 2,2′,2″-(10-(2-((3-((4- (4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)butyl) (methyl)amino)propyl) (methyl)amino)-2- oxoethyl)-1,4,7,10- tetraazacyclododecane- 1,4,7-triyl)triacetic acid | 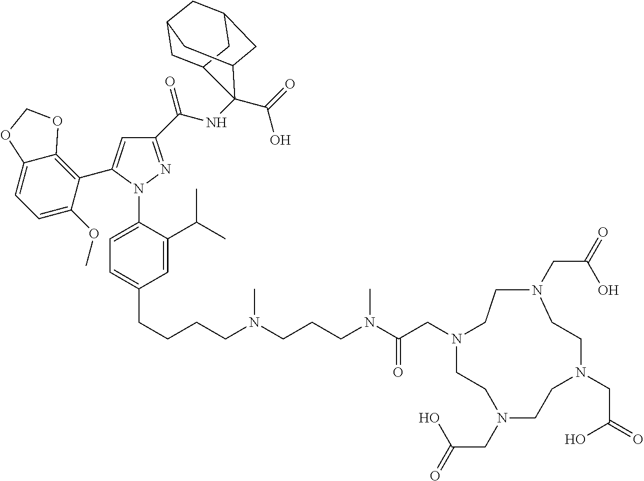 [SMILES: COc1ccc2c(c1-c1cc(C(=O)NC3(C(=O)O)C4CC5CC(C4)CC3C5)nn1-c1ccc(CCCCN(C)CCCN(C)C(=O)CN3CCN(CC(=O)O)CCN(CC(=O)O)CCN(CC(=O)O)CC3)cc1C(C)C)OCO2] | 1101 [M + H]⁺ |
| 29 | 2,2′,2″-(10-(2-(((5S)- 6-((4-((4-(3-(((5r,7r)- 2-Carboxyadamantan- 2-yl)carbamoyl)-5-(5- methoxybenzo[d][1,3] dioxol-4-yl)-1H- pyrazol-1-yl)-3- isopropylphenyl)amino)- 4-oxobutyl)amino)- 5-(4-((4-(4- iodophenyl)butanamido) methyl)benzamido)- 6-oxohexyl)amino)-2- oxoethyl)-1,4,7,10- tetraazacyclododecane- 1,4,7-triyl)triacetic acid | 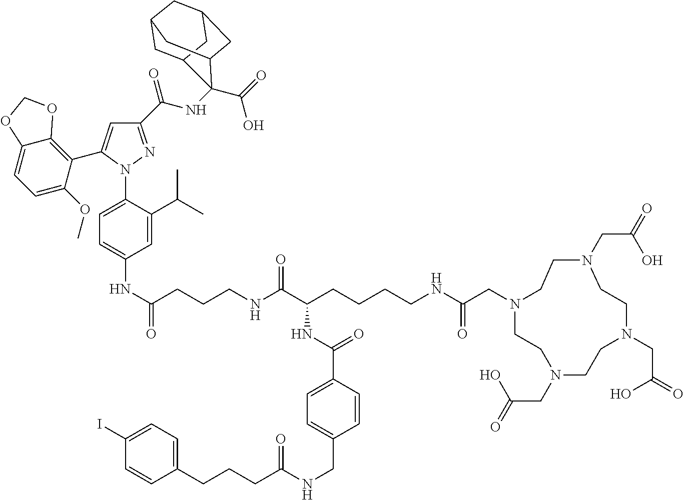 [SMILES: COc1ccc2c(c1-c1cc(C(=O)NC3(C(=O)O)C4CC5CC(C4)CC3C5)nn1-c1ccc(NC(=O)CCCNC(=O)[C@H](CCCCNC(=O)CN3CCN(CC(=O)O)CCN(CC(=O)O)CCN(CC(=O)O)CC3)NC(=O)c3ccc(CNC(=O)CCCc4ccc(I)cc4)cc3)cc1C(C)C)OCO2] | 1578 [M + H]⁺ |
| 30 | 2,2′,2″-(10-((12S)-6- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl) carbamoyl)-12-(4-((4-(4- iodophenyl)butanamido) methyl)benzamido)- 2-methyl-11,18-dioxo- 2,6,10,17- tetraazanonadecan-19- yl)-1,4,7,10- tetraazacyclododecane- 1,4,7-triyl)triacetic acid | 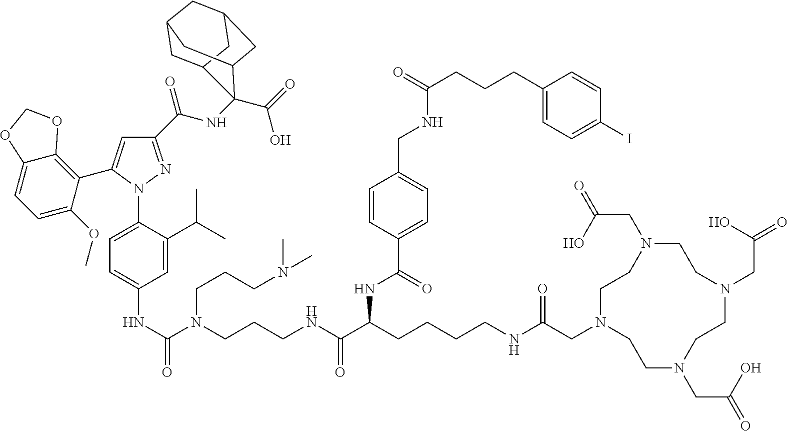 [SMILES: COc1ccc2c(c1-c1cc(C(=O)NC3(C(=O)O)C4CC5CC(C4)CC3C5)nn1-c1ccc(NC(=O)N(CCCNC(=O)[C@H](CCCCNC(=O)CN3CCN(CC(=O)O)CCN(CC(=O)O)CCN(CC(=O)O)CC3)NC(=O)c3ccc(CNC(=O)CCCc4ccc(I)cc4)cc3)CCCN(C)C)cc1C(C)C)OCO2] | 1678 [M + H]⁺ |
| 31 | 2,2′,2″-(10-(6-(4-(3- (((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-12- (4-((4-(4- iodophenyl)butanamido) methyl)benzamido)- 2-methyl-11,18-dioxo- 2,6,10,17- tetraazanonadecan-19- yl)-1,4,7,10- tetraazacyclododecane- 1,4,7-triyl)triacetic acid, Isomer 1 | 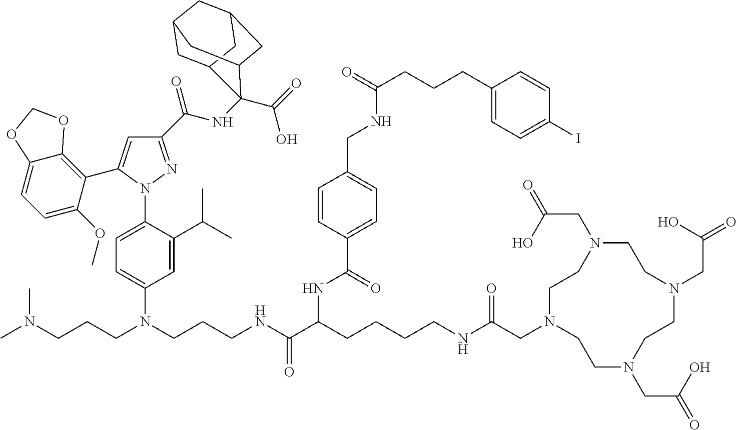 [SMILES: COc1ccc2c(c1-c1cc(C(=O)NC3(C(=O)O)C4CC5CC(C4)CC3C5)nn1-c1ccc(N(CCCNC(=O)C(CCCCNC(=O)CN3CCN(CC(=O)O)CCN(CC(=O)O)CCN(CC(=O)O)CC3)NC(=O)c3ccc(CNC(=O)CCCc4ccc(I)cc4)cc3)CCCN(C)C)cc1C(C)C)OCO2] | 1635 [M + H]⁺ |
| 32 | 2,2′,2″-(10-(6-(4-(3- (((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-12- (4-((4-(4- iodophenyl)butanamido) methyl)benzamido)- 2-methyl-11,18-dioxo- 2,6,10,17- tetraazanonadecan-19- yl)-1,4,7,10- tetraazacyclododecane- 1,4,7-triyl)triacetic acid, Isomer 2 | 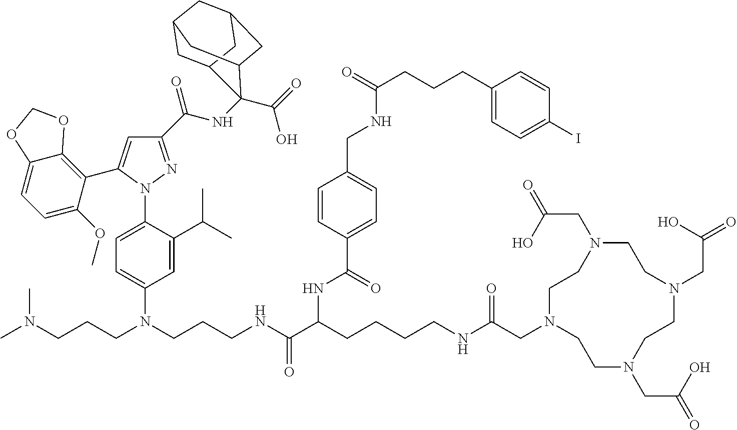 [SMILES: COc1ccc2c(c1-c1cc(C(=O)NC3(C(=O)O)C4CC5CC(C4)CC3C5)nn1-c1ccc(N(CCCNC(=O)C(CCCCNC(=O)CN3CCN(CC(=O)O)CCN(CC(=O)O)CCN(CC(=O)O)CC3)NC(=O)c3ccc(CNC(=O)CCCc4ccc(I)cc4)cc3)CCCN(C)C)cc1C(C)C)OCO2] | 1635/1636 [M + H]⁺ |
| 33 | 2,2′,2″-(10-(2-(4-(3- (((5r,7r)-2- Carboxyadamantan-2- yl)carbamoyl)-5-(5- methoxybenzo[d][1,3] dioxol-4-yl)-1H- pyrazol-1-yl)-3- isopropylphenyl)- 6,10-dimethyl-11,20- dioxo-13,16-dioxa- 2,6,10,19- tetraazahenicosan-21- yl)-1,4,7,10- tetraazacyclododecane- 1,4,7-triyl)triacetic acid | 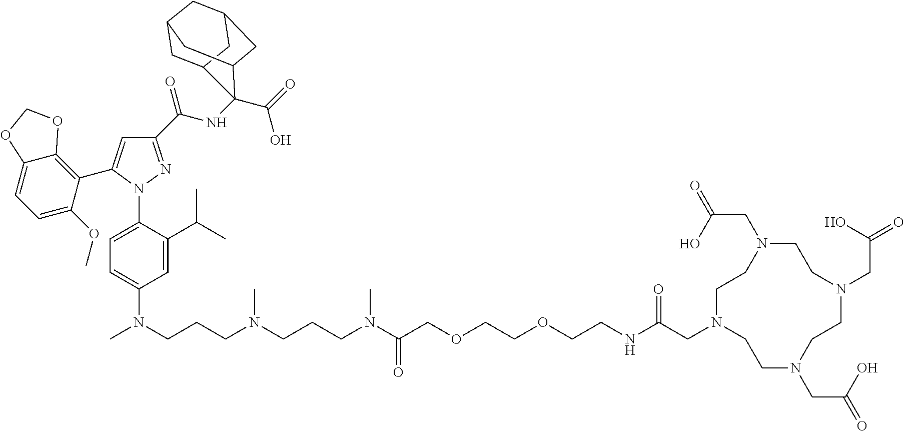 [SMILES: COc1ccc2c(c1-c1cc(C(=O)NC3(C(=O)O)C4CC5CC(C4)CC3C5)nn1-c1ccc(N(C)CCCN(C)CCCN(C)C(=O)COCCOCCNC(=O)CN3CCN(CC(=O)O)CCN(CC(=O)O)CCN(CC(=O)O)CC3)cc1C(C)C)OCO2] | 1261 [M + H]⁺ |
| 34 | 2,2′,2″-(10-(2-((3-((4- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)amino)- 4-oxobutyl)((1- methylpyrrolidin-3- yl)methyl)amino)propyl) (methyl)amino)-2- oxoethyl)-1,4,7,10- tetraazacyclododecane- 1,4,7-triyl)triacetic acid | 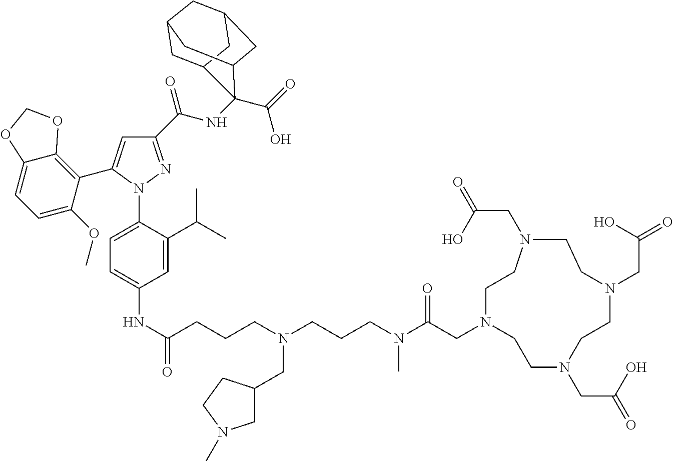 [SMILES: COc1ccc2c(c1-c1cc(C(=O)NC3(C(=O)O)C4CC5CC(C4)CC3C5)nn1-c1ccc(NC(=O)CCCN(CCCN(C)C(=O)CN3CCN(CC(=O)O)CCN(CC(=O)O)CCN(CC(=O)O)CC3)CC3CCN(C)C3)cc1C(C)C)OCO2] | 1213 [M + H]⁺ |
| 53 | 2,2′,2″-(10-(2-((3-(3- (4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenoxy) propoxy)propyl)amino)- 2-oxoethyl)-1,4,7,10- tetraazacyclododecane- 1,4,7-triyl)triacetic acid | 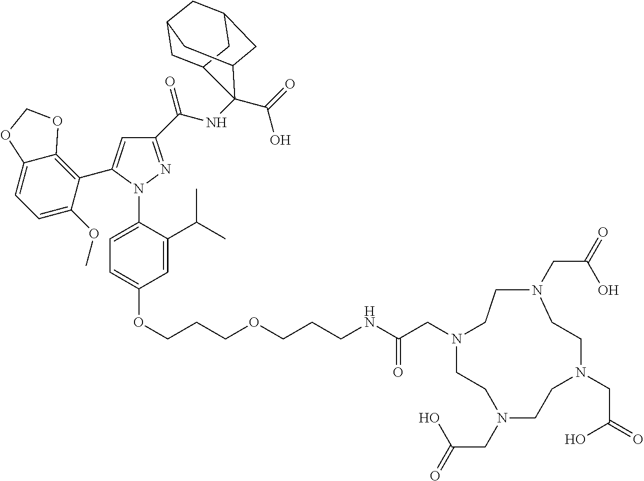 [SMILES: COc1ccc2c(c1-c1cc(C(=O)NC3(C(=O)O)C4CC5CC(C4)CC3C5)nn1-c1ccc(OCCCOCCCNC(=O)CN3CCN(CC(=O)O)CCN(CC(=O)O)CCN(CC(=O)O)CC3)cc1C(C)C)OCO2] | 1076 [M + H]⁺ |
| 60 | 2,2′,2″-(10-(2-((3-((3- (5-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)- 1,3,4-oxadiazol-2- yl)propyl)(methyl) amino)propyl)amino)-2- oxoethyl)-1,4,7,10- tetraazacyclododecane- 1,4,7-triyl)triacetic acid | 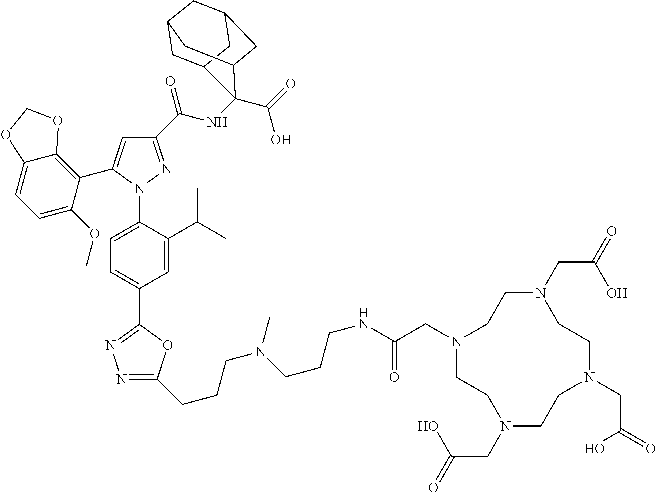 [SMILES: COc1ccc2c(c1-c1cc(C(=O)NC3(C(=O)O)C4CC5CC(C4)CC3C5)nn1-c1ccc(-c3nnc(CCCN(C)CCCNC(=O)CN4CCN(CC(=O)O)CCN(CC(=O)O)CCN(CC(=O)O)CC4)o3)cc1C(C)C)OCO2] | 572 [M/2 + H]⁺ |
| 61 | 2,2′,2″-(10-(2-((3-((3- (4-(3-(((5r,7r)-2- Carboxyadamantan-2- yl)carbamoyl)-5-(5- cyanobenzo[d][1,3] dioxol-4-yl)-1H-pyrazol- 1-yl)-3- isopropylphenoxy) propyl)(methyl)amino) propyl)amino)-2- oxoethyl)-1,4,7,10- tetraazacyclododecane- 1,4,7-triyl)triacetic acid | 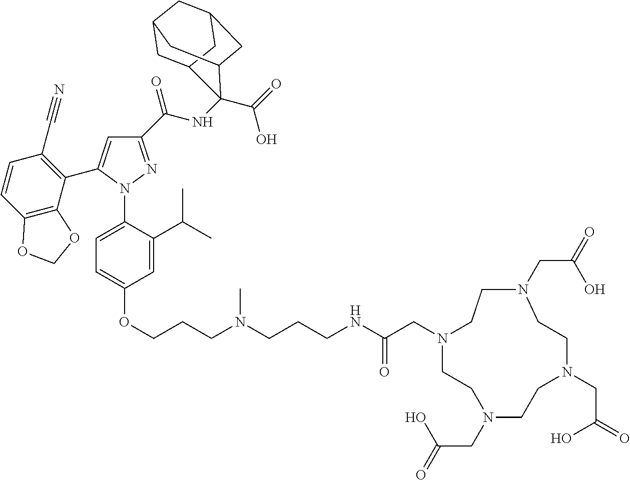 [SMILES: CC(C)c1cc(OCCCN(C)CCCNC(=O)CN2CCN(CC(=O)O)CCN(CC(=O)O)CCN(CC(=O)O)CC2)ccc1-n1nc(C(=O)NC2(C(=O)O)C3CC4CC(C3)CC2C4)cc1-c1c(C#N)ccc2c1OCO2] | 1083 [M + H]⁺ |
| 62 | 2,2′,2″-(10-(2-((3-(4- (2-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenoxy) ethyl)piperazin-1- yl)propyl)amino)-2- oxoethyl)-1,4,7,10- tetraazacyclododecane- 1,4,7-triyl)triacetic acid | 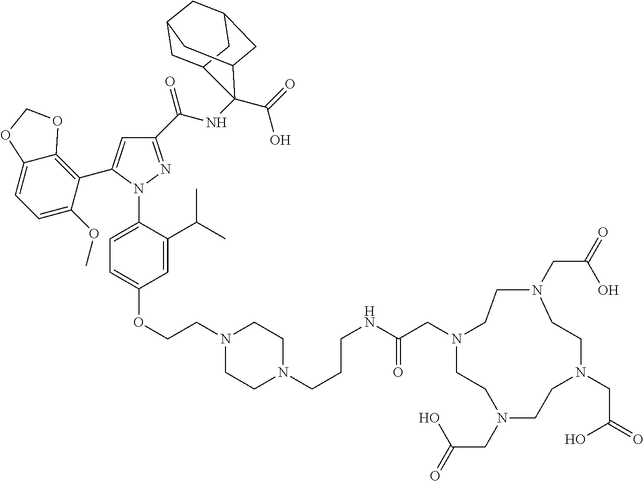 [SMILES: COc1ccc2c(c1-c1cc(C(=O)NC3(C(=O)O)C4CC5CC(C4)CC3C5)nn1-c1ccc(OCCN3CCN(CCCNC(=O)CN4CCN(CC(=O)O)CCN(CC(=O)O)CCN(CC(=O)O)CC4)CC3)cc1C(C)C)OCO2] | 1130 [M + H]⁺ |

The compounds in Table 59 were prepared and the MS data is recorded below. These compounds can be prepared according to Schemes 1-3 or the foregoing Preparations and Examples. Optimum reaction conditions can vary with the particular reactants or solvents used, but such conditions can be determined by one skilled in the art.

TABLE 59

| | | | MS ES+ |
|---|---|---|---|
| Ex # | Chemical Name | Structure | m/z |
| 63 | 2,2′,2″-(10-(2-((3-((3- (7-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)- [1,2,3]triazolo[1,5- a]pyridine-3- carboxamido)propyl) (methyl)amino)propyl) amino)-2-oxoethyl)- 1,4,7,10- tetraazacyclododecane- 1,4,7-triyl)triacetic acid | 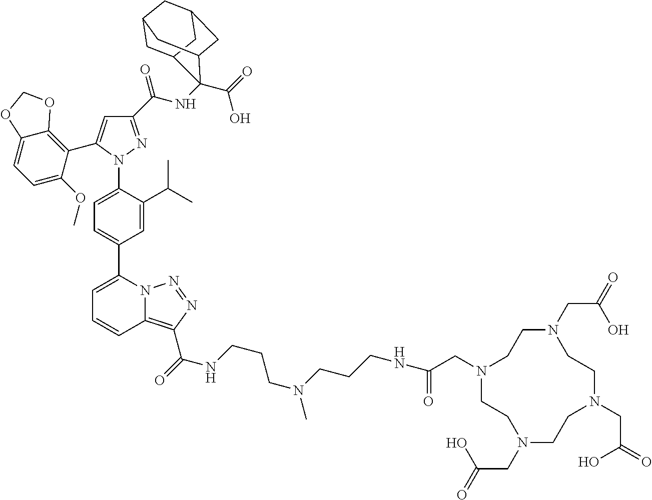 [SMILES: COc1ccc2c(c1-c1cc(C(=O)NC3(C(=O)O)C4CC5CC(C4)CC3C5)nn1-c1ccc(-c3cccc4c(C(=O)NCCCN(C)CCCNC(=O)CN5CCN(CC(=O)O)CCN(CC(=O)O)CCN(CC(=O)O)CC5)nnn34)cc1C(C)C)OCO2] | 1233 [M + H]⁺ |
| 64 | 2,2′,2″-(10-(2-((3-((3- (3-(6-(3-(((5r,7r)-2- Carboxyadamantan-2- yl)carbamoyl)-5-(5- methoxybenzo[d][1,3] dioxol-4-yl)-1H- pyrazol-1-yl)-5- isopropylpyridin-3- yl)ureido)propyl) (methyl)amino)propyl) amino)-2-oxoethyl)- 1,4,7,10- tetraazacyclododecane- 1,4,7-triyl)triacetic acid | 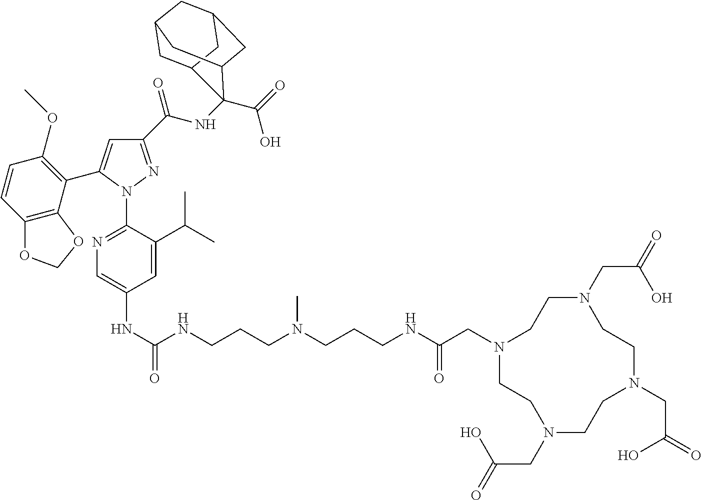 [SMILES: COc1ccc2c(c1-c1cc(C(=O)NC3(C(=O)O)C4CC5CC(C4)CC3C5)nn1-c1ncc(NC(=O)NCCCN(C)CCCNC(=O)CN3CCN(CC(=O)O)CCN(CC(=O)O)CCN(CC(=O)O)CC3)cc1C(C)C)OCO2] | 1132 [M + H]⁺ |
| 65 | 2,2′,2″-(10-(2-((3-(3- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)amino) propoxy)propyl)amino)- 2-oxoethyl)- 1,4,7,10- tetraazacyclododecane- 1,4,7-triyl)triacetic acid | 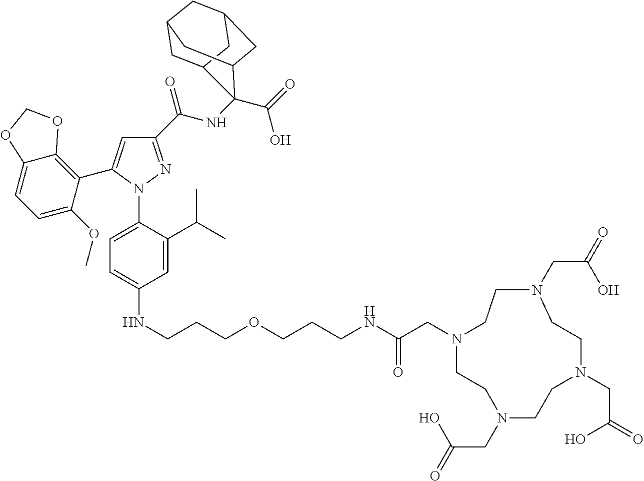 [SMILES: COc1ccc2c(c1-c1cc(C(=O)NC3(C(=O)O)C4CC5CC(C4)CC3C5)nn1-c1ccc(NCCCOCCCNC(=O)CN3CCN(CC(=O)O)CCN(CC(=O)O)CCN(CC(=O)O)CC3)cc1C(C)C)OCO2] | 1075 [M + H]⁺ |
| 66 | 2,2′,2″-(10-(2-((3-((3- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl) amino)-2,2- bis(hydroxymethyl) propyl)amino)propyl) amino)-2-oxoethyl)- 1,4,7,10- tetraazacyclododecane- 1,4,7-triyl)triacetic acid | 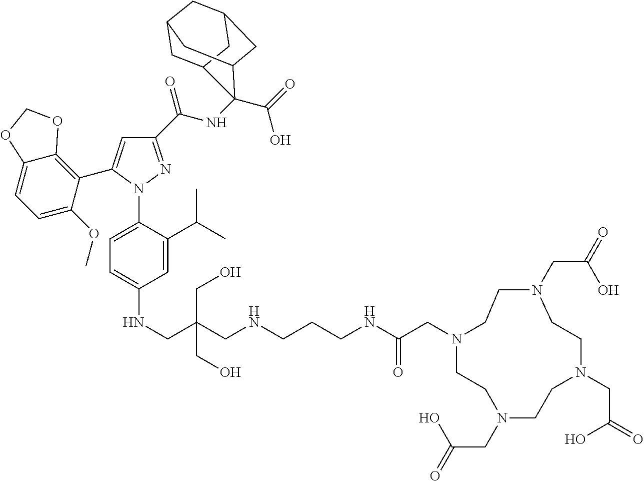 | 1098 [M + H]⁺ |
| 67 | 2,2′,2″-(10-(2-((3-(3- (((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl) amino)methyl)-3- (hydroxymethyl)azetidin- 1-yl)propyl)amino)- 2-oxoethyl)-1,4,7,10- tetraazacyclododecane- 1,4,7-triyl)triacetic acid | 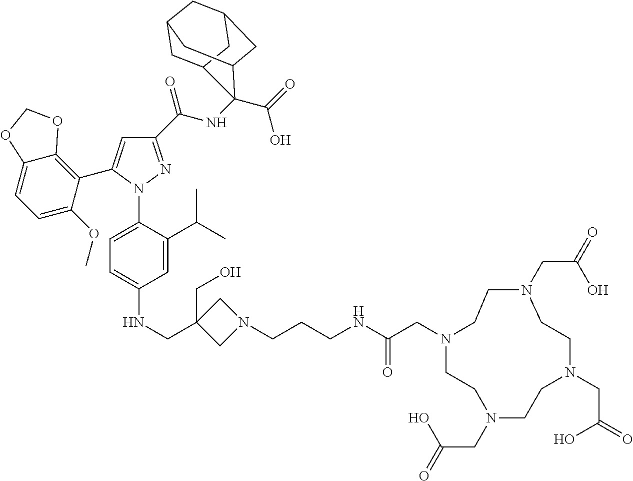 [SMILES: COc1ccc2c(c1-c1cc(C(=O)NC3(C(=O)O)C4CC5CC(C4)CC3C5)nn1-c1ccc(NCC3(CO)CN(CCCNC(=O)CN4CCN(CC(=O)O)CCN(CC(=O)O)CCN(CC(=O)O)CC4)C3)cc1C(C)C)OCO2] | 1098 [M + H]⁺ |
| 68 | 2,2′,2″-(10-(2-((3-((3- (4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenoxy) propyl)(methyl)amino) propyl)amino)-2- oxoethyl)-1,4,7,10- tetraazacyclododecane- 1,4,7-triyl)triacetic acid | 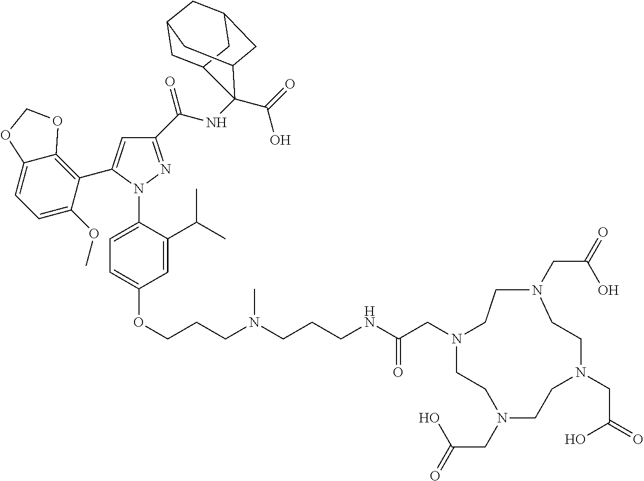 [SMILES: COc1ccc2c(c1-c1cc(C(=O)NC3(C(=O)O)C4CC5CC(C4)CC3C5)nn1-c1ccc(OCCCN(C)CCCNC(=O)CN3CCN(CC(=O)O)CCN(CC(=O)O)CCN(CC(=O)O)CC3)cc1C(C)C)OCO2] | 1089 [M + H]⁺ |
| 69 | 2,2′,2″-(10-(2-((3-((3- (4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenoxy) propyl)(3- (dimethylamino)propyl) amino)propyl)amino)- 2-oxoethyl)-1,4,7,10- tetraazacyclododecane- 1,4,7-triyl)triacetic acid | 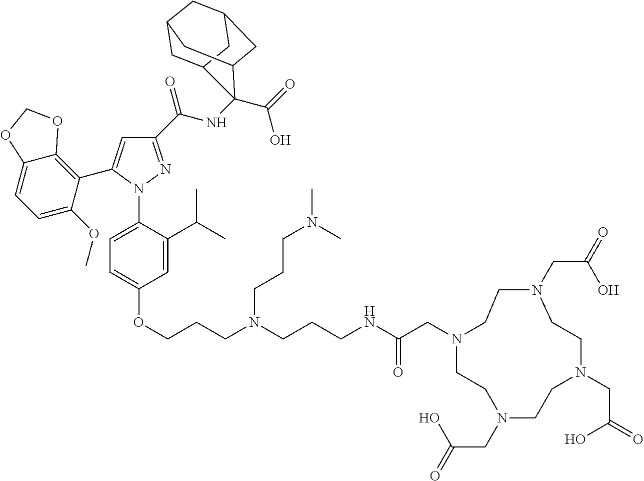 [SMILES: COc1ccc2c(c1-c1cc(C(=O)NC3(C(=O)O)C4CC5CC(C4)CC3C5)nn1-c1ccc(OCCCN(CCCNC(=O)CN3CCN(CC(=O)O)CCN(CC(=O)O)CCN(CC(=O)O)CC3)CCCN(C)C)cc1C(C)C)OCO2] | 1161 [M + H]⁺ |
| 70 | 2,2′,2″-(10-(2-((3-(6- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl) carbamoyl)-2,6- diazaspiro[3.3]heptan- 2-yl)propyl)amino)-2- oxoethyl)-1,4,7,10- tetraazacyclododecane- 1,4,7-triyl)triacetic acid | 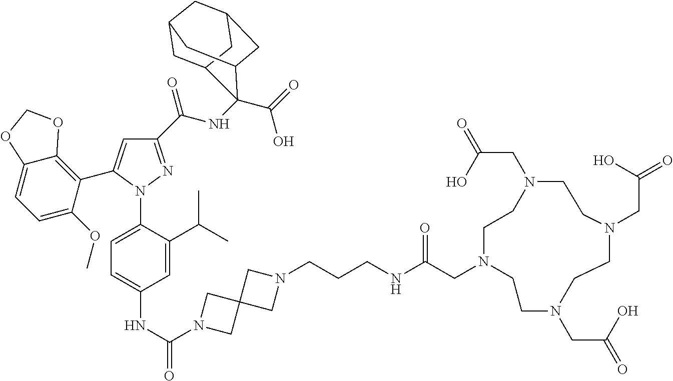 [SMILES: COc1ccc2c(c1-c1cc(C(=O)NC3(C(=O)O)C4CC5CC(C4)CC3C5)nn1-c1ccc(NC(=O)N3CC4(CN(CCCNC(=O)CN5CCN(CC(=O)O)CCN(CC(=O)O)CCN(CC(=O)O)CC5)C4)C3)cc1C(C)C)OCO2] | 1141 [M + H]⁺ |
| 71 | 2,2′,2″-(10-(2-((3-((3- (4-(3-(((S)- ((3S,5S,7S)- Adamantan-1- yl)(carboxy)methyl) carbamoyl)-5-(5- methoxybenzo[d][1,3] dioxol-4-yl)-1H- pyrazol-1-yl)-3- isopropylphenoxy) propyl)(methyl)amino) propyl)amino)-2- oxoethyl)-1,4,7,10- tetraazacyclododecane- 1,4,7-triyl)triacetic acid | 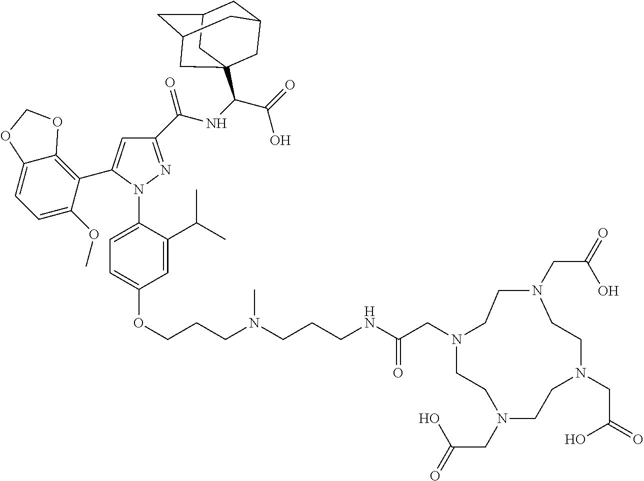 [SMILES: COc1ccc2c(c1-c1cc(C(=O)N[C@H](C(=O)O)C34CC5CC(CC(C5)C3)C4)nn1-c1ccc(OCCCN(C)CCCNC(=O)CN3CCN(CC(=O)O)CCN(CC(=O)O)CCN(CC(=O)O)CC3)cc1C(C)C)OCO2] | 1103 [M + H]⁺ |
| 72 | 2,2′,2″-(10-(2-((3-((3- ((4-(3-(((S)- ((3S,5S,7S)- Adamantan-1- yl)(carboxy)methyl) carbamoyl)-5-(5- methoxybenzo[d][1,3] dioxol-4-yl)-1H- pyrazol-1-yl)-3- isopropylphenyl)amino) propyl)(methyl)amino) propyl)amino)-2- oxoethyl)-1,4,7,10- tetraazacyclododecane- 1,4,7-triyl)triacetic acid | 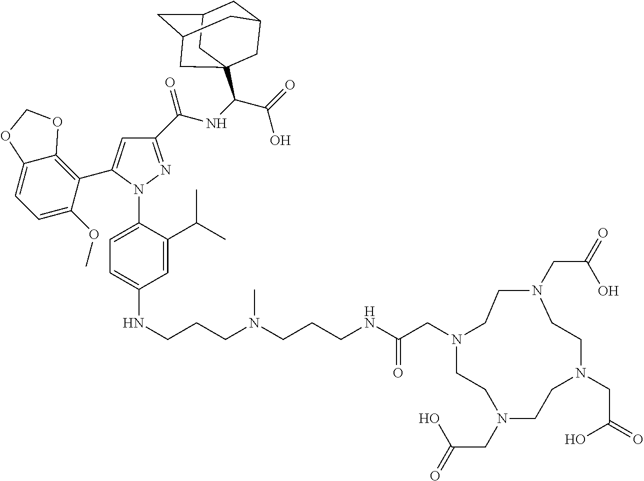 [SMILES: COc1ccc2c(c1-c1cc(C(=O)N[C@H](C(=O)O)C34CC5CC(CC(C5)C3)C4)nn1-c1ccc(NCCCN(C)CCCNC(=O)CN3CCN(CC(=O)O)CCN(CC(=O)O)CCN(CC(=O)O)CC3)cc1C(C)C)OCO2] | 1102 [M + H]⁺ |
| 73 | 2,2′,2″-(10-(2-((3-((3- (3-(4-(3-(((S)- ((3S,5S,7S)- Adamantan-1- yl)(carboxy)methyl) carbamoyl)-5-(5- methoxybenzo[d][1,3] dioxol-4-yl)-1H- pyrazol-1-yl)-3- isopropylphenyl)ureido) propyl)(methyl)amino) propyl)amino)-2- oxoethyl)-1,4,7,10- tetraazacyclododecane- 1,4,7-triyl)triacetic acid | 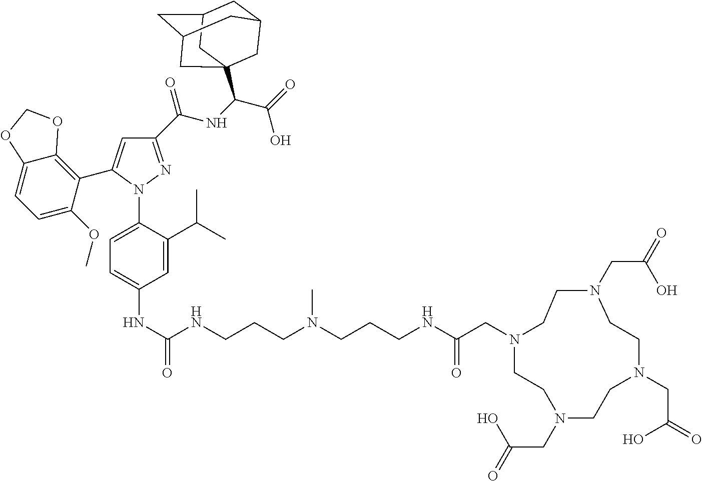 [SMILES: COc1ccc2c(c1-c1cc(C(=O)N[C@H](C(=O)O)C34CC5CC(CC(C5)C3)C4)nn1-c1ccc(NC(=O)NCCCN(C)CCCNC(=O)CN3CCN(CC(=O)O)CCN(CC(=O)O)CCN(CC(=O)O)CC3)cc1C(C)C)OCO2] | 1145 [M + H]⁺ |
| 74 | 2,2′,2″-(10-(2-((3-(4- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenoxy) methyl)piperidin-1- yl)propyl)amino)-2- oxoethyl)-1,4,7,10- tetraazacyclododecane- 1,4,7-triyl)triacetic acid | 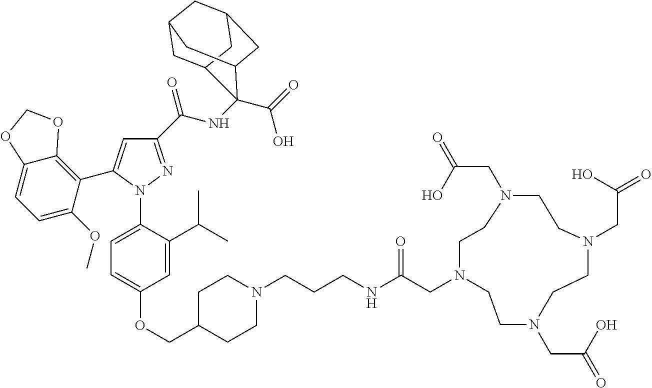 [SMILES: COc1ccc2c(c1-c1cc(C(=O)NC3(C(=O)O)C4CC5CC(C4)CC3C5)nn1-c1ccc(OCC3CCN(CCCNC(=O)CN4CCN(CC(=O)O)CCN(CC(=O)O)CCN(CC(=O)O)CC4)CC3)cc1C(C)C)OCO2] | 558 [M/2 + H]⁺ |
| 75 | 2,2′,2″-(10-(2-(4-(3-(4- (2-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenoxy) ethyl)piperazin-1- yl)propyl)piperazin-1- yl)-2-oxoethyl)- 1,4,7,10- tetraazacyclododecane- 1,4,7-triyl)triacetic acid | 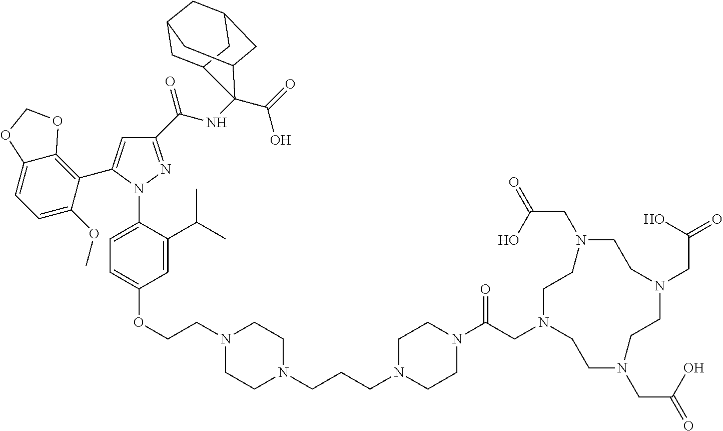 [SMILES: COc1ccc2c(c1-c1cc(C(=O)NC3(C(=O)O)C4CC5CC(C4)CC3C5)nn1-c1ccc(OCCN3CCN(CCCN4CCN(C(=O)CN5CCN(CC(=O)O)CCN(CC(=O)O)CCN(CC(=O)O)CC5)CC4)CC3)cc1C(C)C)OCO2] | 1198 [M + H]⁺ |
| 76 | 2,2′,2″-(10-(2-((3-((3- (3-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)- [1,2,3]triazolo[1,5- a]pyridine-7- carboxamido)propyl) (methyl)amino)propyl) amino)-2-oxoethyl)- 1,4,7,10- tetraazacyclododecane- 1,4,7-triyl)triacetic acid | 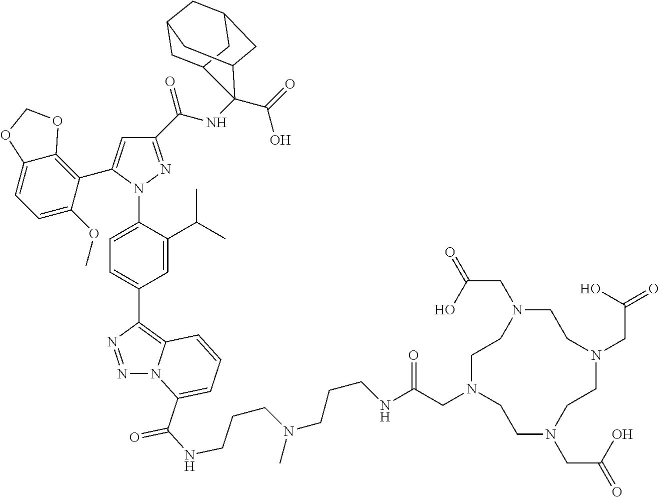 [SMILES: COc1ccc2c(c1-c1cc(C(=O)NC3(C(=O)O)C4CC5CC(C4)CC3C5)nn1-c1ccc(-c3nnn4c(C(=O)NCCCN(C)CCCNC(=O)CN5CCN(CC(=O)O)CCN(CC(=O)O)CCN(CC(=O)O)CC5)cccc34)cc1C(C)C)OCO2] | 1233 [M + H]⁺ |
| 77 | 2,2′,2″-(10-(2-((3-(6- (4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-2,6- diazaspiro[3.3]heptan- 2-yl)propyl)amino)-2- oxoethyl)-1,4,7,10- tetraazacyclododecane- 1,4,7-triyl)triacetic acid | 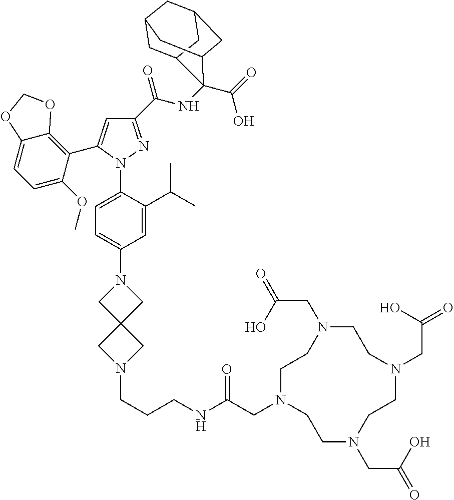 [SMILES: COc1ccc2c(c1-c1cc(C(=O)NC3(C(=O)O)C4CC5CC(C4)CC3C5)nn1-c1ccc(N3CC4(CN(CCCNC(=O)CN5CCN(CC(=O)O)CCN(CC(=O)O)CCN(CC(=O)O)CC5)C4)C3)cc1C(C)C)OCO2] | 1098 [M + H]⁺ |
| 78 | 2,2′,2″-(10-(2-((3-((4- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl) amino)-4-oxobutyl)(3- (dimethylamino)propyl) amino)propyl)(methyl) amino)-2-oxoethyl)- 1,4,7,10- tetraazacyclododecane- 1,4,7-triyl)triacetic acid | 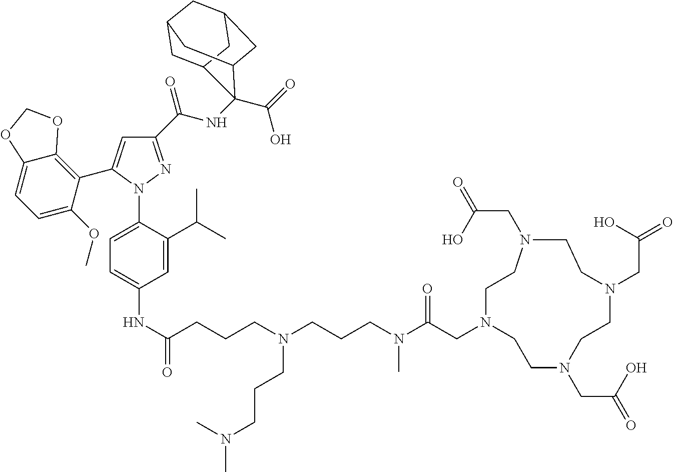 | 1187 [M + H]⁺ |
| 79 | 2,2′,2″-(10-(2-((3-((3- ((6-(3-(((5r,7r)-2- Carboxyadamantan-2- yl)carbamoyl)-5-(5- methoxybenzo[d][1,3] dioxol-4-yl)-1H- pyrazol-1-yl)-5- isopropylpyridin-3- yl)oxy)propyl)(methyl) amino)propyl)amino)- 2-oxoethyl)-1,4,7,10- tetraazacyclododecane- 1,4,7-triyl)triacetic acid | 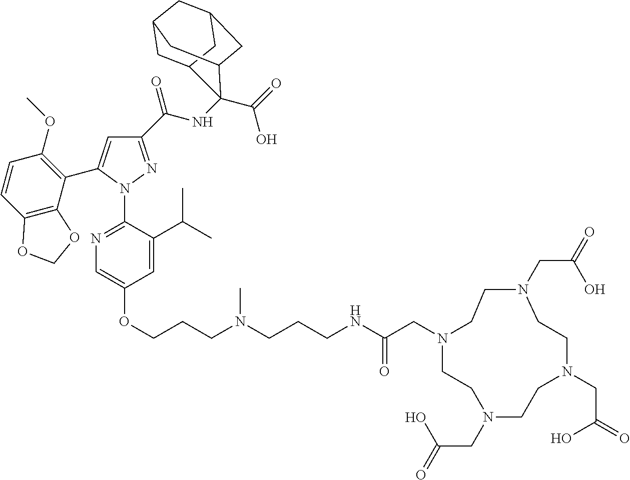 [SMILES: COc1ccc2c(c1-c1cc(C(=O)NC3(C(=O)O)C4CC5CC(C4)CC3C5)nn1-c1ncc(OCCCN(C)CCCNC(=O)CN3CCN(CC(=O)O)CCN(CC(=O)O)CCN(CC(=O)O)CC3)cc1C(C)C)OCO2] | 1090 [M + H]⁺ |
| 80 | 2,2′,2″-(10-(2-((3-((3- ((5-(3-(((5r,7r)-2- Carboxyadamantan-2- yl)carbamoyl)-5-(5- methoxybenzo[d][1,3] dioxol-4-yl)-1H- pyrazol-1-yl)-6- isopropylpyridin-2- yl)oxy)propyl)(methyl) amino)propyl)amino)- 2-oxoethyl)-1,4,7,10- tetraazacyclododecane- 1,4,7-triyl)triacetic acid | 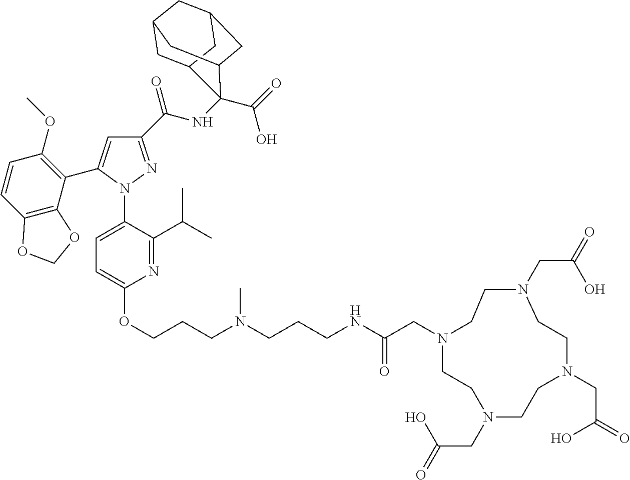 [SMILES: COc1ccc2c(c1-c1cc(C(=O)NC3(C(=O)O)C4CC5CC(C4)CC3C5)nn1-c1ccc(OCCCN(C)CCCNC(=O)CN3CCN(CC(=O)O)CCN(CC(=O)O)CCN(CC(=O)O)CC3)nc1C(C)C)OCO2] | 1090 [M + H]⁺ |
| 81 | 2,2′,2″-(10-(2-((3-(3- (3-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl)ureido) propoxy)propyl)amino)- 2-oxoethyl)- 1,4,7,10- tetraazacyclododecane- 1,4,7-triyl)triacetic acid | 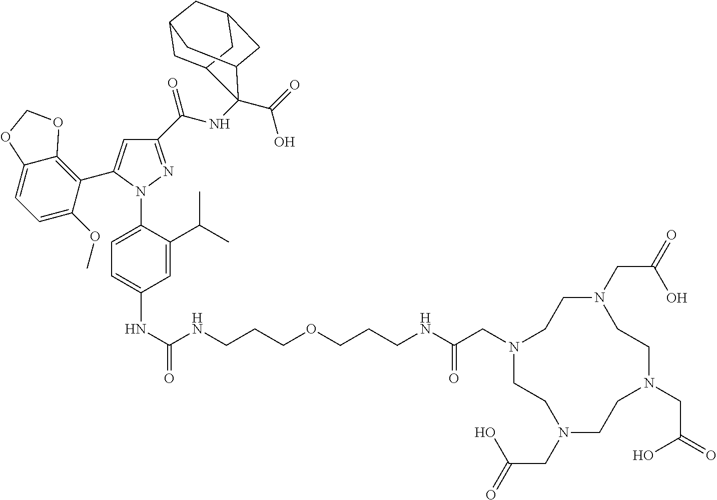 [SMILES: COc1ccc2c(c1-c1cc(C(=O)NC3(C(=O)O)C4CC5CC(C4)CC3C5)nn1-c1ccc(NC(=O)NCCCOCCCNC(=O)CN3CCN(CC(=O)O)CCN(CC(=O)O)CCN(CC(=O)O)CC3)cc1C(C)C)OCO2] | 1118 [M + H]⁺ |
| 82 | 2,2′,2″-(10-(2-((3-((3- (3-(5-(3-(((5r,7r)-2- Carboxyadamantan-2- yl)carbamoyl)-5-(5- methoxybenzo[d][1,3] dioxol-4-yl)-1H- pyrazol-1-yl)-6- isopropylpyridin-2- yl)ureido)propyl) (methyl)amino)propyl) amino)-2-oxoethyl)- 1,4,7,10- tetraazacyclododecane- 1,4,7-triyl)triacetic acid | 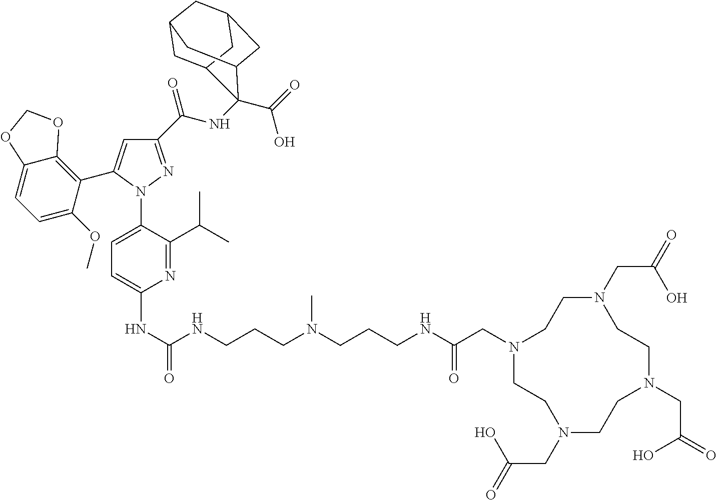 [SMILES: COc1ccc2c(c1-c1cc(C(=O)NC3(C(=O)O)C4CC5CC(C4)CC3C5)nn1-c1ccc(NC(=O)NCCCN(C)CCCNC(=O)CN3CCN(CC(=O)O)CCN(CC(=O)O)CCN(CC(=O)O)CC3)nc1C(C)C)OCO2] | 1132 [M + H]⁺ |
| 83 | 2,2′,2″-(10-(2-(4-(3- ((4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl) amino)-3- oxopropyl)piperazin- 1-yl)-2-oxoethyl)- 1,4,7,10- tetraazacyclododecane- 1,4,7-triyl)triacetic acid | 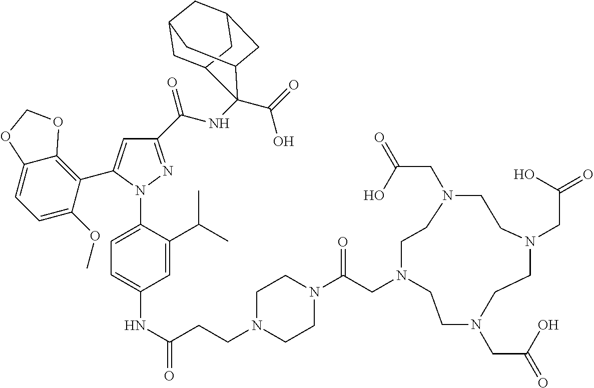 [SMILES: COc1ccc2c(c1-c1cc(C(=O)NC3(C(=O)O)C4CC5CC(C4)CC3C5)nn1-c1ccc(NC(=O)CCN3CCN(C(=O)CN4CCN(CC(=O)O)CCN(CC(=O)O)CCN(CC(=O)O)CC4)CC3)cc1C(C)C)OCO2] | 1100 [M + H]⁺ |
| 84 | 2,2′,2″-(10-(2-(((1-(3- (3-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl) ureido)propyl)-1H- imidazol-4- yl)methyl)amino)-2- oxoethyl)-1,4,7,10- tetraazacyclododecane- 1,4,7-triyl)triacetic acid | 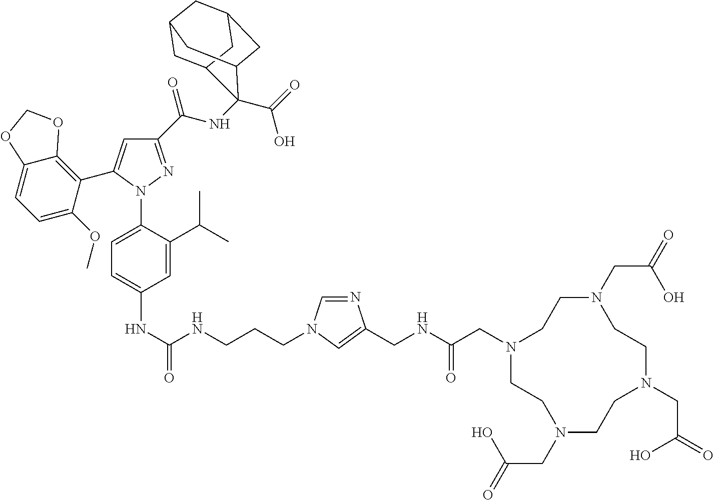 [SMILES: COc1ccc2c(c1-c1cc(C(=O)NC3(C(=O)O)C4CC5CC(C4)CC3C5)nn1-c1ccc(NC(=O)NCCCn3cnc(CNC(=O)CN4CCN(CC(=O)O)CCN(CC(=O)O)CCN(CC(=O)O)CC4)c3)cc1C(C)C)OCO2] | 1140 [M + H]⁺ |
| 85 | 2,2′,2″-(10-(2-(((1-(3- (3-(4-(3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl) ureido)propyl)-3- fluoroazetidin-3- yl)methyl)amino)-2- oxoethyl)-1,4,7,10- tetraazacyclododecane- 1,4,7-triyl)triacetic acid | 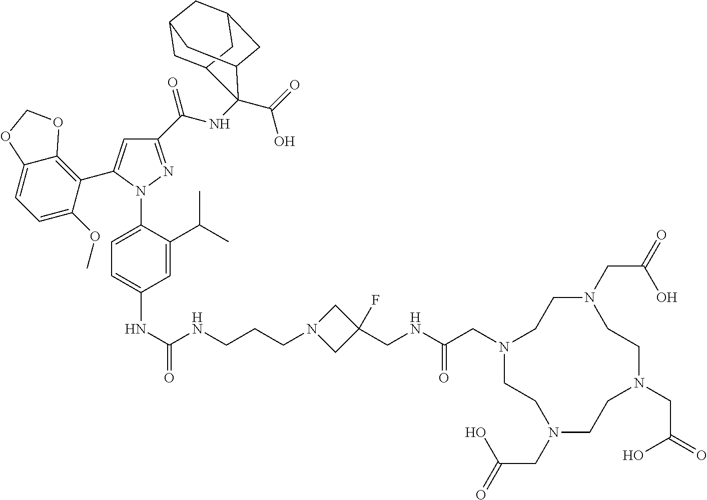 [SMILES: COc1ccc2c(c1-c1cc(C(=O)NC3(C(=O)O)C4CC5CC(C4)CC3C5)nn1-c1ccc(NC(=O)NCCCN3CC(F)(CNC(=O)CN4CCN(CC(=O)O)CCN(CC(=O)O)CCN(CC(=O)O)CC4)C3)cc1C(C)C)OCO2] | 1147 [M + H]⁺ |
| 86 | 2,2′,2″-(10-(2-((3-((3- (3-(4-(3-(((5r,7r)-2- Carboxyadamantan-2- yl)carbamoyl)-5-(5- cyanobenzo[d][1,3] dioxol-4-yl)-1H- pyrazol-1-yl)-3- isopropylphenyl)ureido) propyl)(methyl)amino) propyl)amino)-2- oxoethyl)-1,4,7,10- tetraazacyclododecane- 1,4,7-triyl)triacetic acid | 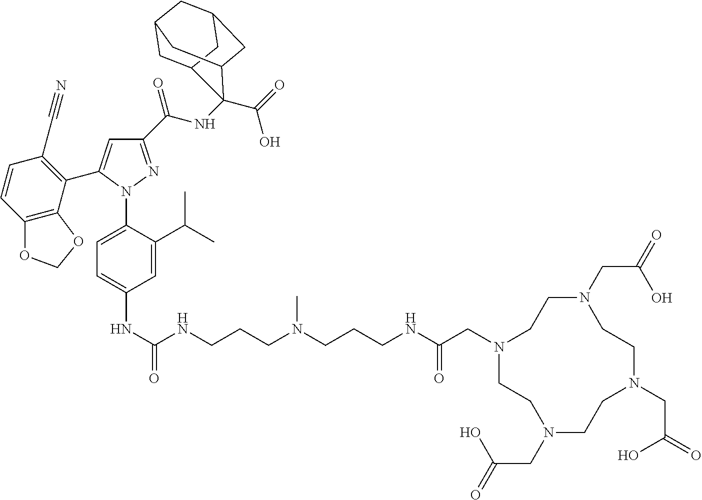 [SMILES: CC(C)c1cc(NC(=O)NCCCN(C)CCCNC(=O)CN2CCN(CC(=O)O)CCN(CC(=O)O)CCN(CC(=O)O)CC2)ccc1-n1nc(C(=O)NC2(C(=O)O)C3CC4CC(C3)CC2C4)cc1-c1c(C#N)ccc2c1OCO2] | 1125 [M + H]⁺ |

### Example 1-Lu

### Lutetium (III) 2,2′,2″-(10-(2-((3-((3-(4-(3-(((5r,7r)-2-Carboxyadamantan-2-yl)carbamoyl)-5-(5-methoxybenzo[d][1,3]dioxol-4-yl)-1H-pyrazol-1-yl)-3-isopropylphenoxy)propyl)(methyl)amino)propyl)(methyl)amino)-2-oxoethyl)-1,4,7,10-tetraazacyclododecane-1,4,7-triyl)triacetic acid

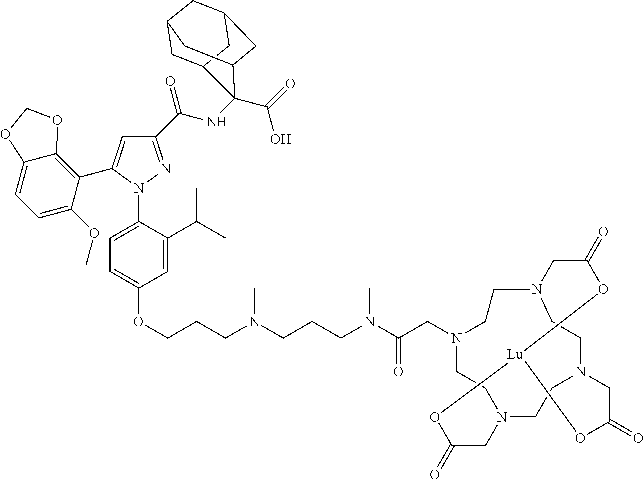

A soln of 2,2′,2″-(10-(2-((3-((3-(4-(3-(((5r,7r)-2-carboxyadamantan-2-yl)carbamoyl)-5-(5-methoxybenzo[d][1,3]dioxol-4-yl)-1H-pyrazol-1-yl)-3-isopropylphenoxy)propyl)(methyl)amino)propyl)(methyl)amino)-2-oxoethyl)-1,4,7,10-tetraazacyclododecane-1,4,7-triyl)triacetic acid (17 mg, 0.015 mmol) in pH 5 NaOAc/AcOH buffer (2 mL) was treated with lutetium (III) chloride (13 mg, 0.046 mmol) in portions at RT. The pH of the soln was adjusted to a pH between 4 and 5 with sat. NaHCO₃ solution. After stirring at 80° C. for 30 min the solution was diluted by DMF (1 mL), filtered, and purified by prep-HPLC with the following conditions: Column, Xselect CSH C18 OBD 50*250 mm, 5 μm; Mobile Phase, 20% to 40% ACN in H₂O (10 mM NH₄OAc) to afford title compound (17 mg, 87%) as a white solid. MS ES+ m/z 1275 [M+H]⁺.

The following compounds were prepared in a manner essentially analogous to the preparation method of Lutetium (III) 2,2′,2″-(10-(2-((3-((3-(4-(3-(((5r,7r)-2-Carboxyadamantan-2-yl)carbamoyl)-5-(5-methoxybenzo[d][1,3]dioxol-4-yl)-1H-pyrazol-1-yl)-3-isopropylphenoxy)propyl)(methyl)amino)propyl)(methyl)amino)-2-oxoethyl)-1,4,7,10-tetraazacyclododecane-1,4,7-triyl)triacetic acid using the appropriate reagents, adjusting temperature, adjusting reaction time to determine completion of the reaction, and adjusting the purification system as appropriate.

TABLE 21

| | | | MS ES+ |
|---|---|---|---|
| Ex # | Chemical Name | Structure | m/z |
| 2-Lu | Lutetium(III) 2,2′,2″- (10-(2-((2-((3-(4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenoxy) propyl)(methyl) amino)ethyl)amino)- 2-oxoethyl)-1,4,7,10- tetraazacyclo- dodecane-1,4,7- triyl)triacetic acid | 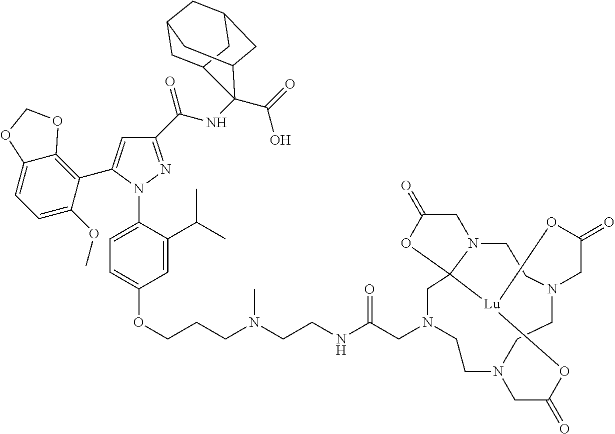 | 1247 [M + H]⁺ |
| 3-Lu | Lutetium(III) 2,2′,2″- (10-(2-((4-((2-((3-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenoxy) propyl)(methyl)amino) ethyl)amino)-4- oxobutyl)(methyl) amino)-2-oxoethyl)- 1,4,7,10- tetraazacyclododecane- 1,4,7-triyl)triacetic acid | 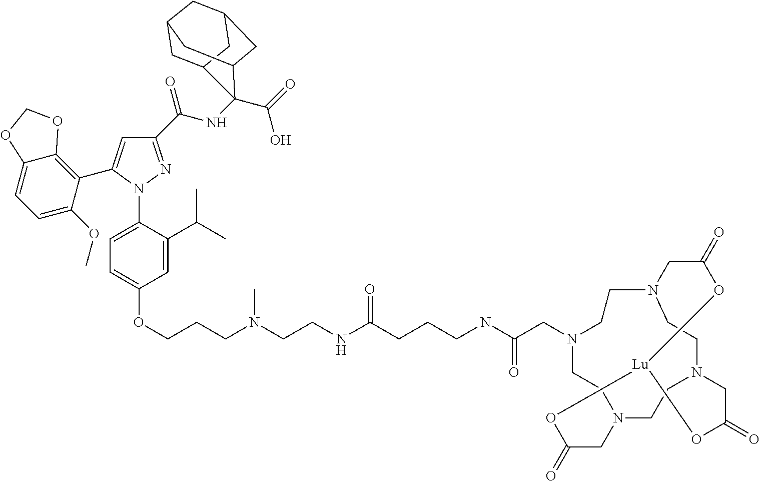 | 1346 [M + H]⁺ |
| 4-Lu | Lutetium(III) 2,2′,2″- (10-(2-((3-((4-((4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl) amino)-4- oxobutyl)(methyl) amino)propyl)(methyl) amino)-2-oxoethyl)- 1,4,7,10- tetraazacyclododecane- 1,4,7-triyl)triacetic acid | 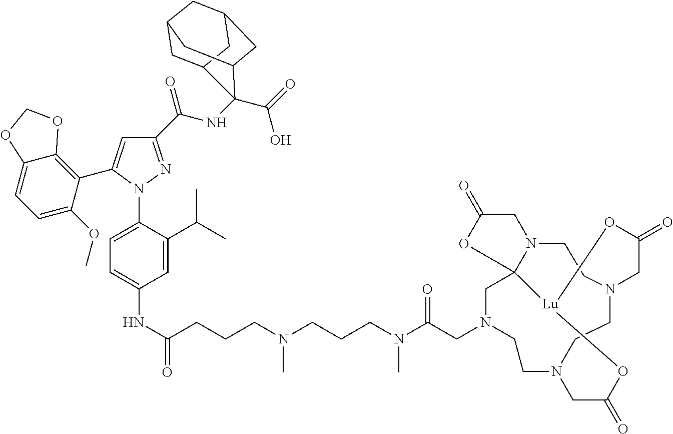 | 1302 [M + H]⁺ |
| 5-Lu | Lutetium(III) 2,2′,2″- (10-(2-((3-((3-((N-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl) sulfamoyl)(methyl) amino)propyl)(methyl) amino)propyl)(methyl) amino)-2-oxoethyl)- 1,4,7,10- tetraazacyclododecane- 1,4,7-triyl)triacetic acid | 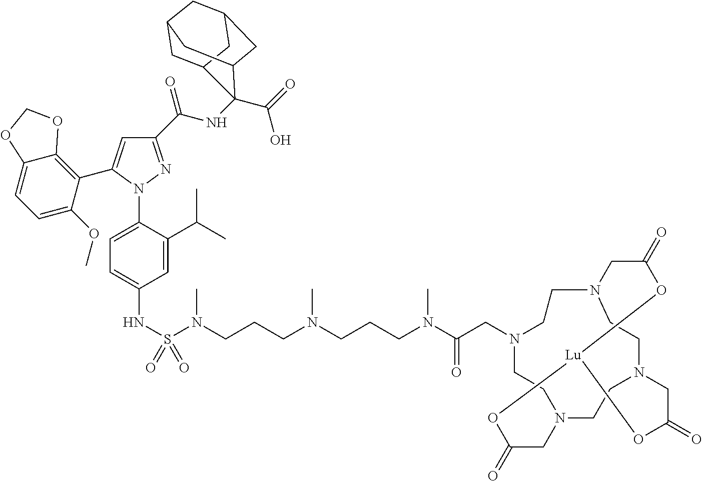 | 1367 [M + H]⁺ |
| 6-Lu | Lutetium(III) 2,2′,2″- (10-(2-((3-((4-((4′-(3- (((5r,7r)-2- Carboxyadamantan-2- yl)carbamoy1)-5-(5- methoxybenzo[d][1,3] dioxol-4-yl)-1H- pyrazol-1-yl)-3′- isopropyl-[1,1′- bipheny1]-4- yl)amino)-4- oxobutyl)(methyl) amino)propyl) (methyl)amino)- 2-oxoethyl)- 1,4,7,10- tetraazacyclododecane- 1,4,7-triyl)triacetic acid | 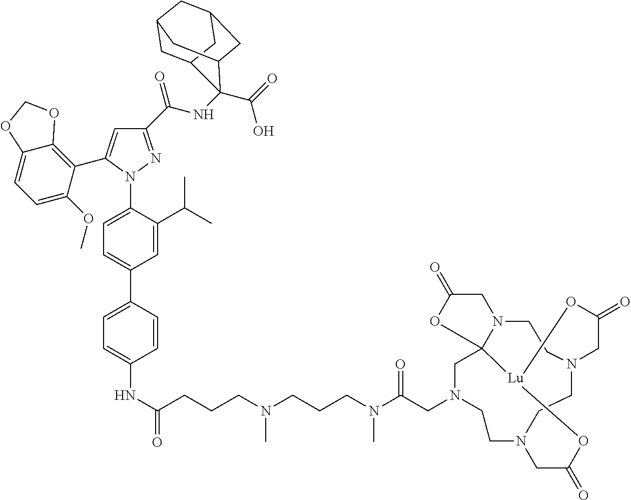 | 1378 [M + H]⁺ |
| 7-Lu | Lutetium(III) 2,2′,2″- (10-(2-((3-((3-((4-(3- (((5r,7r)-2- Carboxyadamantan-2- yl)carbamoy1)-5-(5- methoxybenzo[d][1,3] dioxol-4-y1)-1H- pyrazol-1-yl)-3- isopropylphenyl) (methyl)amino) propyl)(methyl) amino)propyl) (methyl)amino)-2- oxoethyl)-1,4,7,10- tetraazacyclododecane- 1,4,7-triyl)triacetic acid | 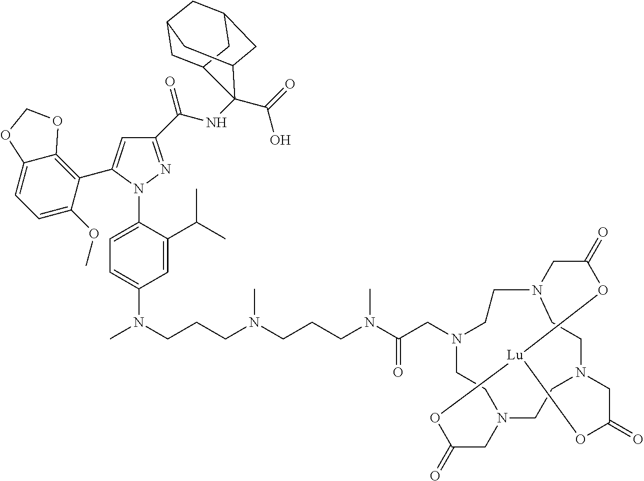 | 1288 [M + H]⁺ |
| 8-Lu | Lutetium(III) 2,2′,2″- (10-(2-((3-((3-(3-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl)-1- methylureido)propyl) (methyl)amino)propyl) (methyl)amino)-2- oxoethyl)-1,4,7,10- tetraazacyclododecane- 1,4,7-triyl)triacetic acid | 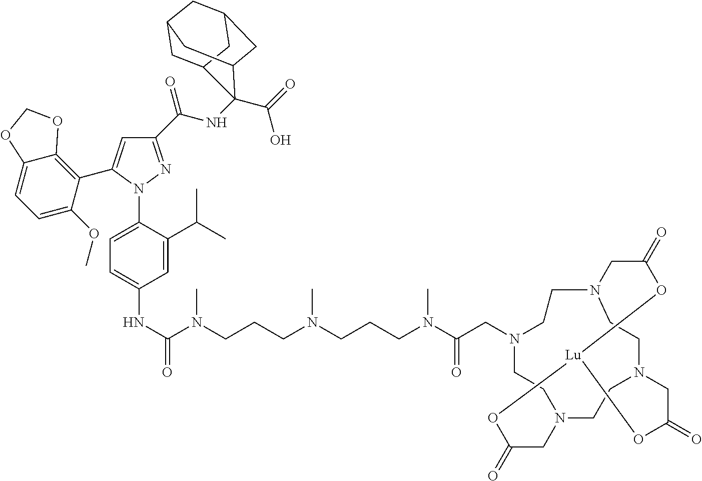 | 1331 [M + H]⁺ |
| 9-Lu | Lutetium(III) 2,2′,2″- (10-(2-((3-((3-((4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl) amino)propyl)(methyl) amino)propyl)amino)- 2-oxoethyl)-1,4,7,10- tetraazacyclododecane- 1,4,7-triyl)triacetic acid | 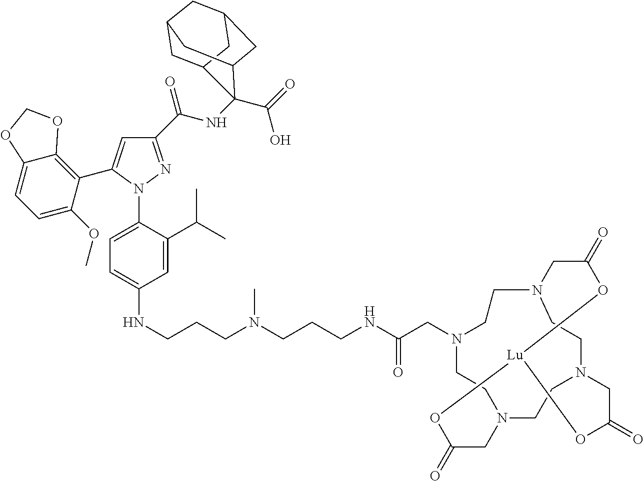 | 1260 [M + H]⁺ |
| 10-Lu | Lutetium(III) 2,2′,2″- (10-(2-((3-((3-(((4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl) carbamoyl)oxy)propyl) (methyl)amino)propyl) (methyl)amino)-2- oxoethyl)-1,4,7,10- tetraazacyclododecane- 1,4,7-triyl)triacetic acid | 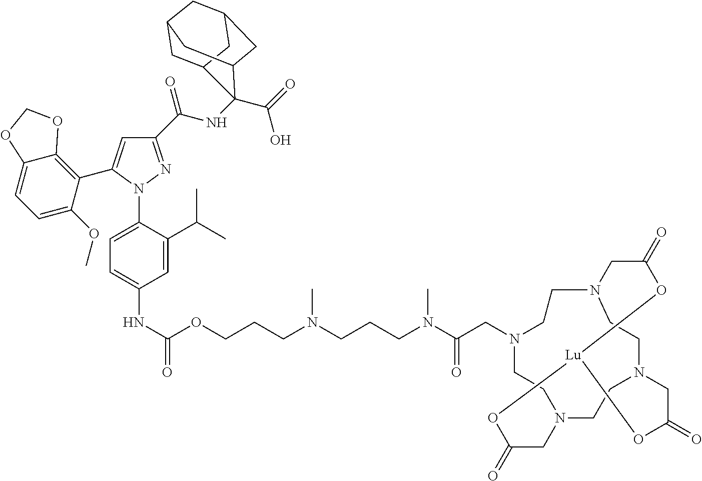 | 1318 [M + H]⁺ |
| 11-Lu | Lutetium(III) 2,2′,2″- (10-(2-((3-((3-(3-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl) ureido)propyl)(methyl) amino)propyl)(methyl) amino)-2-oxoethyl)- 1,4,7,10- tetraazacyclododecane- 1,4,7-triyl)triacetic acid | 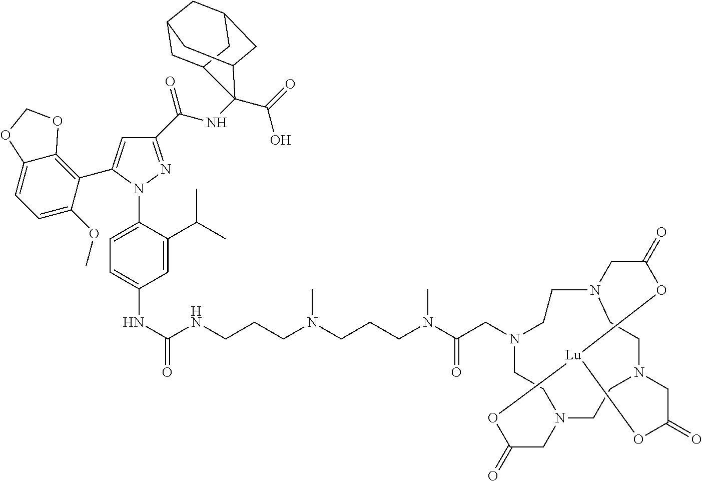 | 1317 [M + H]⁺ |
| 12-Lu | Lutetium(III) 2,2′,2″- (10-(2-((3-((3-(4-(4- (3-(((5r,7r)-2-(tert- Butoxycarbonyl) adamantan-2- yl)carbamoyl)-5-(5- methoxybenzo[d][1,3] dioxol-4-y1)-1H- pyrazol-1-yl)-3- isopropylphenyl)-1H- 1,2,3-triazol-1- yl)propyl)(methyl) amino)propyl)(methyl) amino)-2-oxoethyl)- 1,4,7,10- tetraazacyclododecane- 1,4,7-triyl)triacetic acid | 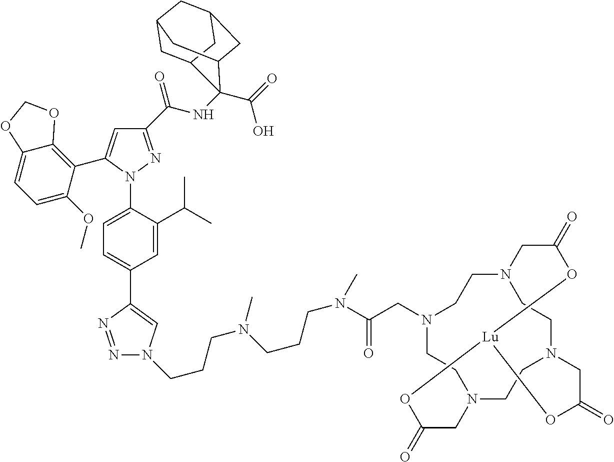 | 1326 [M + H]⁺ |
| 13-Lu | Lutetium(III) 2,2′,2″- (10-(2-((3-((4-((4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl) amino)-4- oxobutyl)(methyl) amino)propyl) amino)-2- oxoethyl)-1,4,7,10- tetraazacyclododecane- 1,4,7-triyl)triacetic acid | 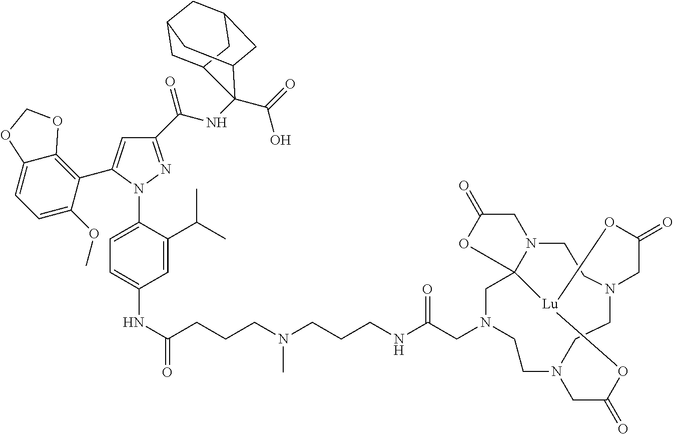 | 1288 [M + H]⁺ |
| 14-Lu | Lutetium(III) 2,2′,2″- (10-(2-((3-((3-(4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenoxy) propyl)(methyl)amino) propyl)amino)-2- oxoethyl)-1,4,7,10- tetraazacyclododecane- 1,4,7-triyl)triacetic acid | 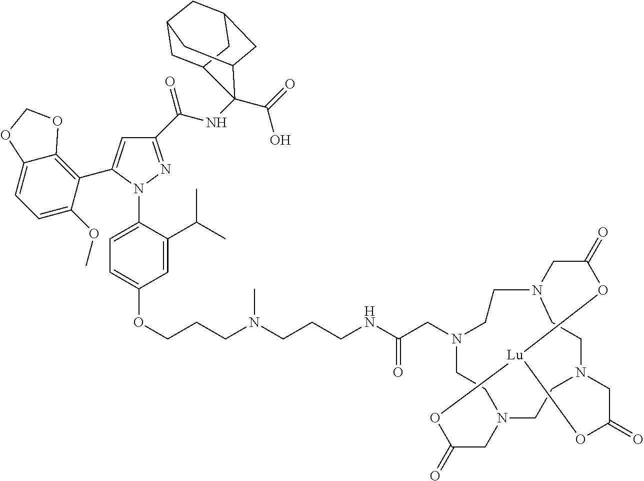 | 1261 [M + H]⁺ |
| 15-Lu | Lutetium(III) 2,2′,2″- (10-(17-((4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl) amino)-2,17-dioxo- 6,9,12,15-tetraoxa-3- azaheptadecyl)- 1,4,7,10- tetraazacyclododecane- 1,4,7-triyl)triacetic acid | 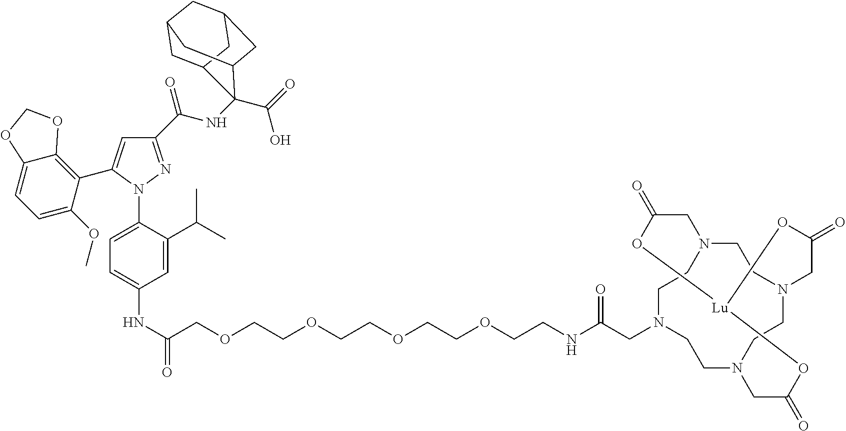 | 1365 [M + H]⁺ |
| 16-Lu | Lutetium(III) 2,2′,2″- (10-(2-((3-(4-(2-((4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl) amino)-2- oxoethyl)piperidin-1- yl)propyl)amino)-2- oxoethyl)-1,4,7,10- tetraazacyclododecane- 1,4,7-triyl)triacetic acid | 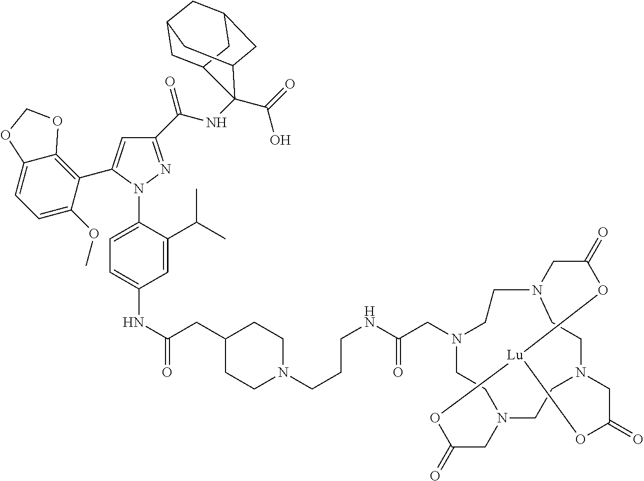 | 1314 [M + H]⁺ |
| 17-Lu | Lutetium(III) 2,2′,2″- (10-(3-(3-((3-((4-(3- (((5r,7r)-2- Carboxyadamantan-2- yl)carbamoyl)-5-(5- methoxybenzo[d][1,3] dioxol-4-yl)-1H- pyrazol-1-yl)-3- isopropylphenyl) (methyl)amino)propyl) (methyl)amino)propoxy) propyl)-1,4,7,10- tetraazacyclododecane- 1,4,7-triyl)triacetic acid | 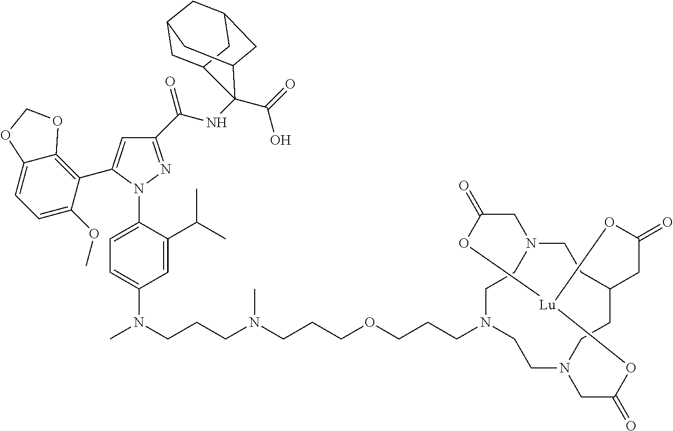 | 1275 [M + H]⁺ |
| 18-Lu | Lutetium(III) 2,2′,2″- (10-(2-((2-((2-(4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenoxy) ethyl)(methyl)amino) ethy1)amino)-2- oxoethyl)-1,4,7,10- tetraazacyclododecane- 1,4,7-triyl)triacetic acid | 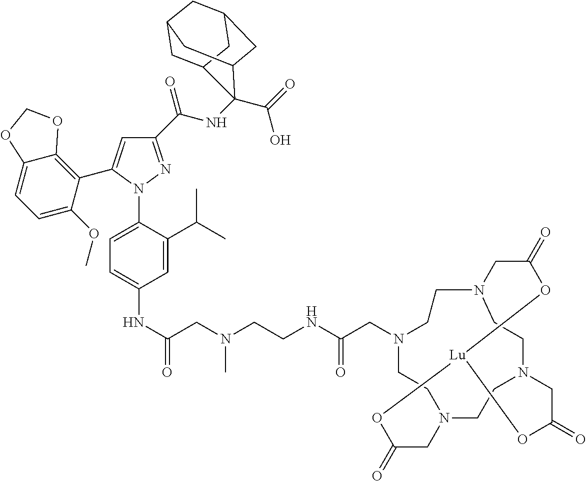 | 1233 [M + H]⁺ |
| 19-Lu | Lutetium(III) 2,2′,2″- (10-(2-((2-((2-(((4-(3- (((5r,7r)-2- Carboxyadamantan-2- yl)carbamoyl)-5-(5- methoxybenzo[d][1,3] dioxol-4-y1)-1H- pyrazol-1-y1)-3- isopropylphenyl) carbamoyl)oxy)ethyl) (methyl)amino)ethyl) amino)-2-oxoethyl)- 1,4,7,10- tetraazacyclododecane- 1,4,7-triyl)triacetic acid | 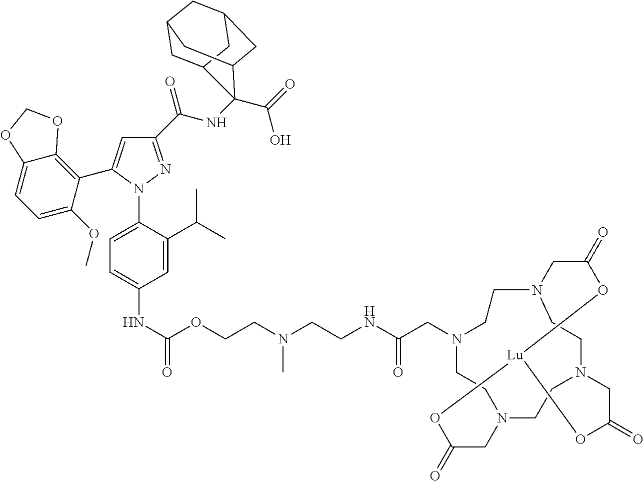 | 1276 [M + H]⁺ |
| 20-Lu | Lutetium(III) 2,2′,2″- (10-(2-((3-((3-(3-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl) ureido)propyl)(methyl) amino)propyl)amino)- 2-oxoethyl)-1,4,7,10- tetraazacyclododecane- 1,4,7-triyl)triacetic acid | 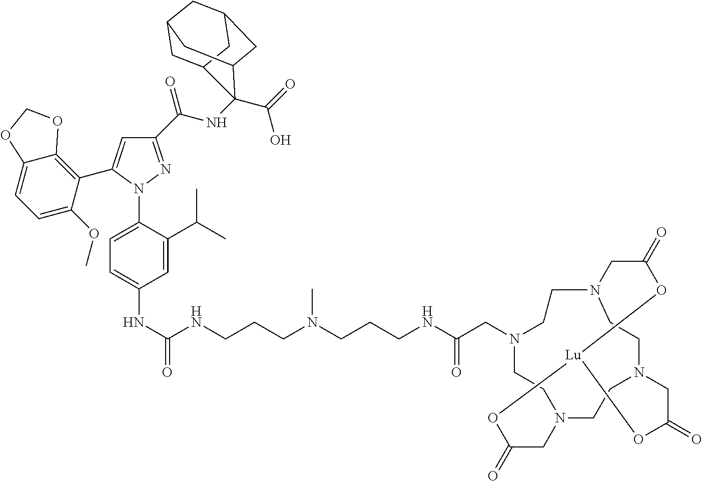 | 1303 [M + H]⁺ |
| 21-Lu | Lutetium(III) 2,2′,2″- (10-(2-((3-((3-(5-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl) picolinamido)propyl) (methy1)amino)propyl) amino)-2-oxoethyl)- 1,4,7,10- tetraazacyclododecane- 1,4,7-triyl)triacetic acid | 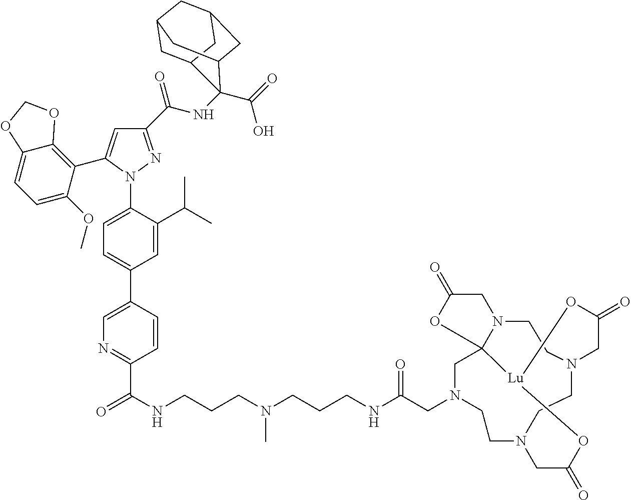 | 1365 [M + H]⁺ |
| 22-Lu | Lutetium(III) 2,2′,2″- (10-(2-((3-((3-(4-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl)-1H- 1,2,3-triazol-1- yl)propyl)(methyl) amino)propyl)amino)-2- oxoethyl)-1,4,7,10- tetraazacyclododecane- 1,4,7-triyl)triacetic acid | 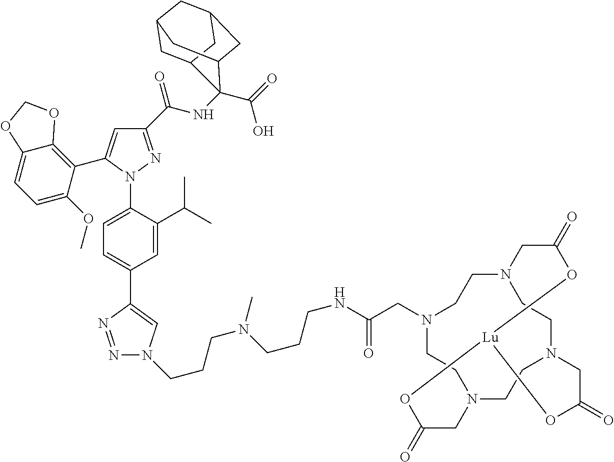 | 1312 [M + H]⁺ |
| 23-Lu | Lutetium(III) 2,2′,2″′- (10-(2-((3-(3-(4-(4-(3- (((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-1H- 1,2,3-triazol-1- yl)propoxy)propyl) amino)-2-oxoethyl)- 1,4,7,10- tetraazacyclododecane- 1,4,7-triyl)triacetic acid | 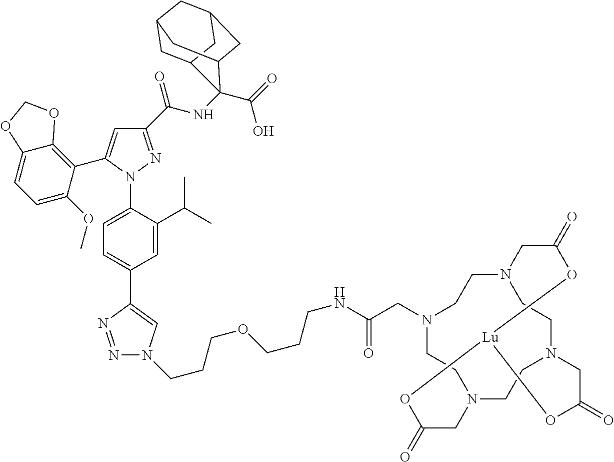 | 1299 [M + H]⁺ |
| 24-Lu | Lutetium(III) 2,2′,2″- (10-(1-Carboxy-4-((3- ((3-(3-(4-(3-(((5r,7r)- 2-carboxyadamantan- 2-yl)carbamoyl)-5-(5- methoxybenzo[d][1,3] dioxol-4-y1)-1H- pyrazol-1-y1)-3- isopropylphenyl)-1- methylureido)propyl) (methyl)amino)propyl) (methyl)amino)-4- oxobutyl)-1,4,7,10- tetraazacyclododecane- 1,4,7-triyl)triacetic acid | 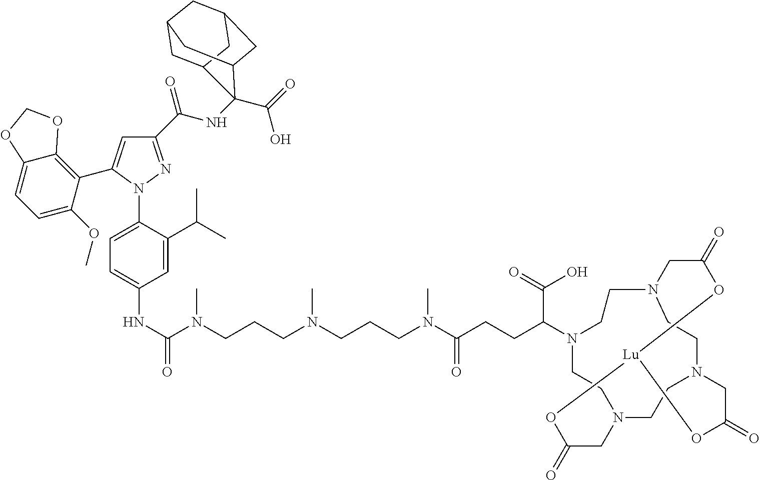 | 1403 [M + H]⁺ |
| 25-Lu | Lutetium(III) 2,2′,2″- (10-(2-((3-((3-(4-(5- (Benzo[1,2-d:4,5- d′]bis([1,3]dioxole)-4- yl)-3-(((5r,7r)-2- carboxyadamantan-2- yl)carbamoyl)-1H- pyrazol-1-y1)-3- isopropyl-N- methylbenzamido) propyl)(methyl)amino) propyl)(methyl) amino)-2- oxoethyl)-1,4,7,10- tetraazacyclododecane- 1,4,7-triyl)triacetic acid | 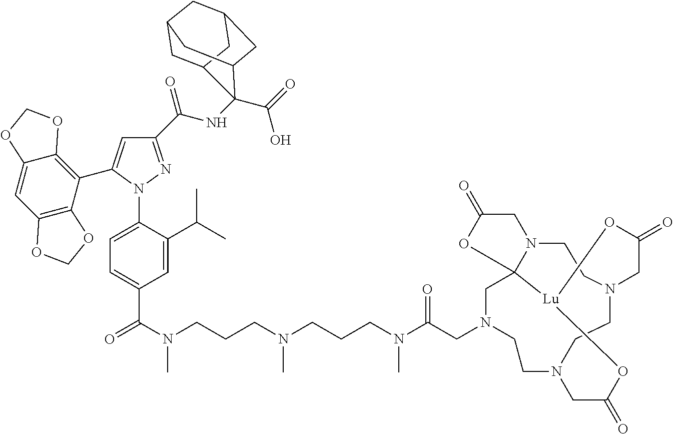 | 1330 [M + H]⁺ |
| 26-Lu | Lutetium(III) 2,2′,2″- (10-(2-((3-((3-(3-(4- (3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl) quinoline-7- carboxamido)propyl) (methyl)amino)propyl) amino)-2-oxoethyl)- 1,4,7,10- tetraazacyclododecane- 1,4,7-triyl)triacetic acid | 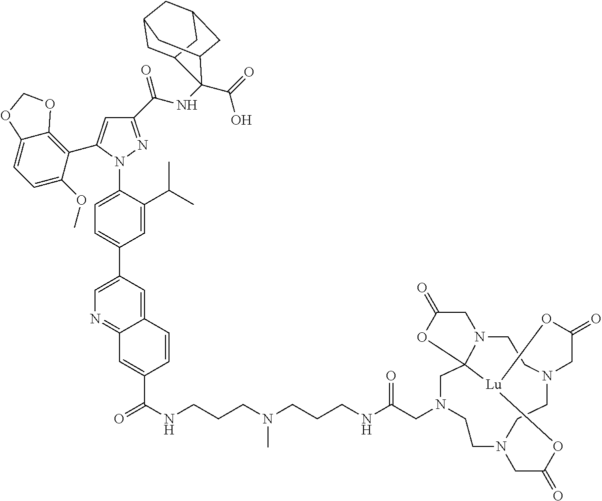 | 1415 [M + H]⁺ |
| 27-Lu | Lutetium(III) 2,2′,2″- (10-(2-((3-((4-((4-(3- (((5r,7r)-2- Carboxyadamantan-2- yl)carbamoyl)-5-(5- methoxybenzo[d][1,3] dioxol-4-yl)-1H- pyrazol-1-yl)-3- isopropylphenyl) amino)-4-oxobutyl)(3- (dimethylamino)propy1) amino)propyl)(methy 1)amino)-2-oxoethyl)- 1,4,7,10- tetraazacyclododecane- 1,4,7-triyl)triacetic acid | 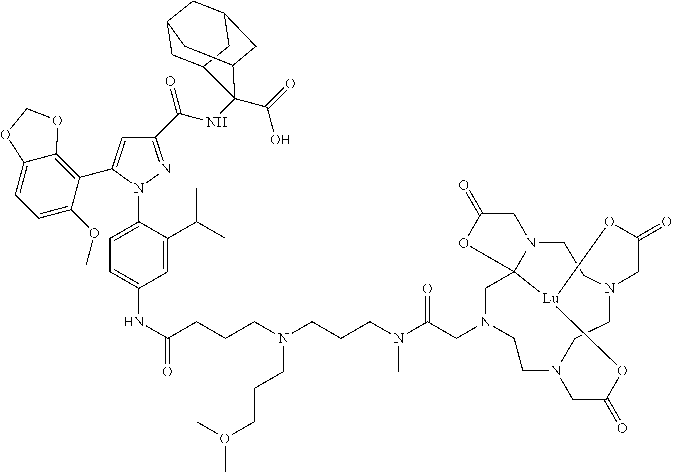 | 1374 [M + H]⁺ |
| 28-Lu | Lutetium(III) 2,2′,2″- (10-(2-((3-((4-(4-(3- (((5r,7r)-2- Carboxyadamantan-2- yl)carbamoyl)-5-(5- methoxybenzo[d][1,3] dioxol-4-yl)-1H- pyrazol-1-yl)-3- isopropylphenyl)butyl) (methyl)amino)propyl) (methyl)amino)-2- oxoethyl)-1,4,7,10- tetraazacyclododecane- 1,4,7-triyl)triacetic acid; formic acid | 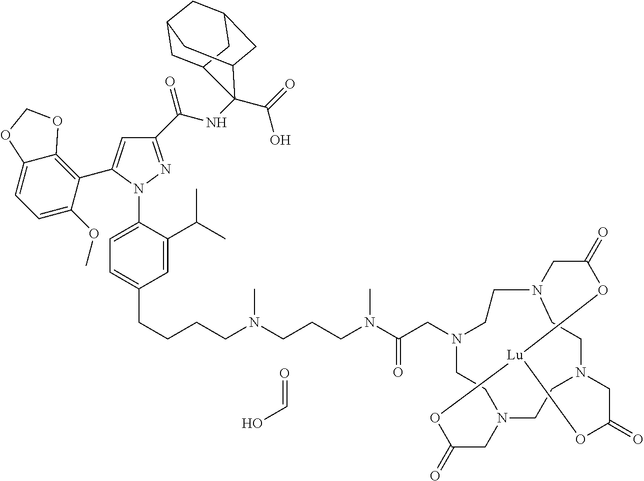 | 1273 [M + H]⁺ |
| 29-Lu | Lutetium(III) 2,2′,2″- (10-(2-(((5S)-6-((4- ((4-(3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl) amino)-4-oxobutyl) amino)-5-(4-((4-(4- iodophenyl)butanamido) methyl)benzamido)- 6-oxohexyl)amino)-2- oxoethyl)-1,4,7,10- tetraazacyclododecane- 1,4,7-triyl)triacetic acid | 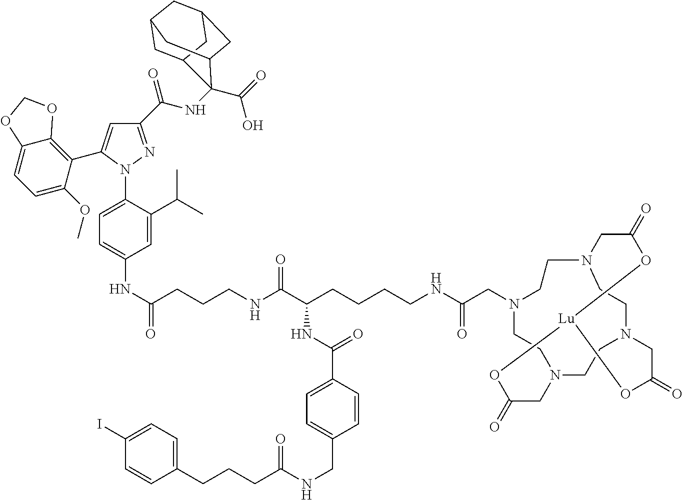 | 1750 [M + H]⁺ |
| 30-Lu | Lutetium(III) 2,2′,2″- (10-((12S)-6-((4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl) carbamoyl)-12-(4- ((4-(4-iodophenyl) butanamido)methyl) benzamido)- 2-methyl-11,18-dioxo- 2,6,10,17- tetraazanonadecan-19- yl)-1,4,7,10- tetraazacyclododecane- 1,4,7-triyl)triacetic acid | 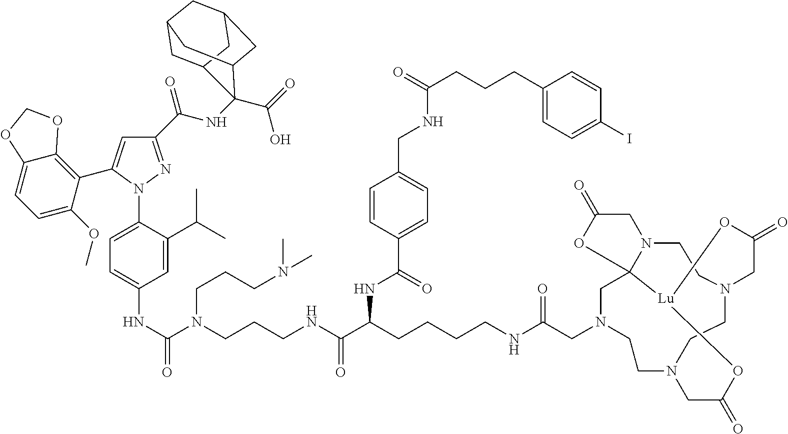 | 1850 [M + H]⁺ |
| 31-Lu | Lutetium(III) 2,2′,2″- (10-(6-(4-(3-(((5r,7r)- 2-Carboxyadamantan- 2-yl)carbamoyl)-5-(5- methoxybenzo[d][1,3] dioxol-4-y1)-1H- pyrazol-1-yl)-3- isopropylphenyl)-12- (4-((4-(4-iodophenyl) butanamido)methyl) benzamido)- 2-methyl-11,18-dioxo- 2,6,10,17- tetraazanonadecan-19- yl)-1,4,7,10- tetraazacyclododecane- 1,4,7-triyl)triacetic acid, Isomer 1 | 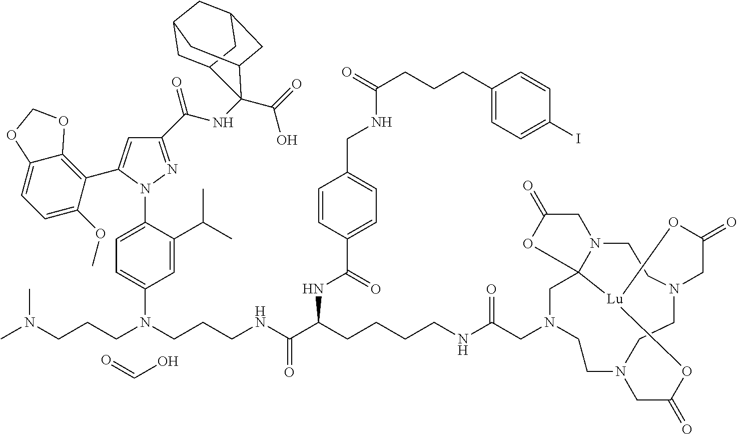 | 1807/1808 [M + H]⁺ |
| 32-Lu | Lutetium(III) 2,2′,2″- (10-(6-(4-(3-(((5r,7r)- 2-Carboxyadamantan- 2-yl)carbamoyl)-5-(5- methoxybenzo[d][1,3] dioxol-4-y1)-1H- pyrazol-1-yl)-3- isopropylphenyl)-12- (4-((4-(4-iodophenyl) butanamido)methyl) benzamido)- 2-methyl-11,18-dioxo- 2,6,10,17- tetraazanonadecan-19- yl)-1,4,7,10- tetraazacyclododecane- 1,4,7-triyl)triacetic acid, Isomer 2 | 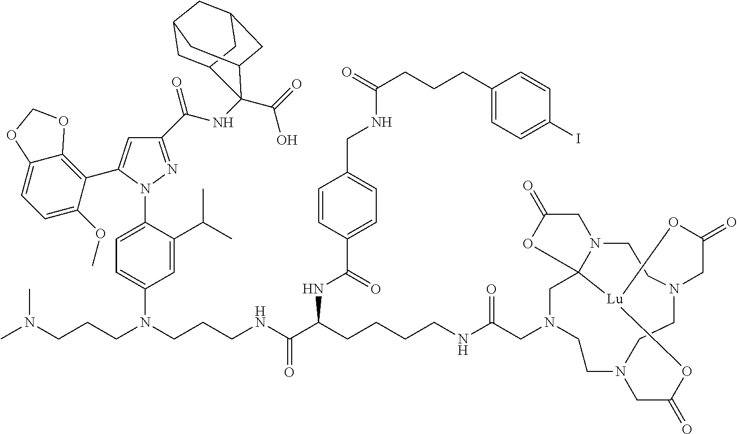 | 1807/1808 [M + H]⁺ |
| 33-Lu | Lutetium(III) 2,2′,2″- (10-(2-(4-(3-(((5r,7r)- 2-Carboxyadamantan- 2-yl)carbamoyl)-5-(5- methoxybenzo[d][1,3] dioxol-4-yl)-1H- pyrazol-1-yl)-3- isopropylphenyl)- 6,10-dimethyl-11,20- dioxo-13,16-dioxa- 2,6,10,19- tetraazahenicosan-21- yl)-1,4,7,10- tetraazacyclododecane- 1,4,7-triyl)triacetic acid; formic acid | 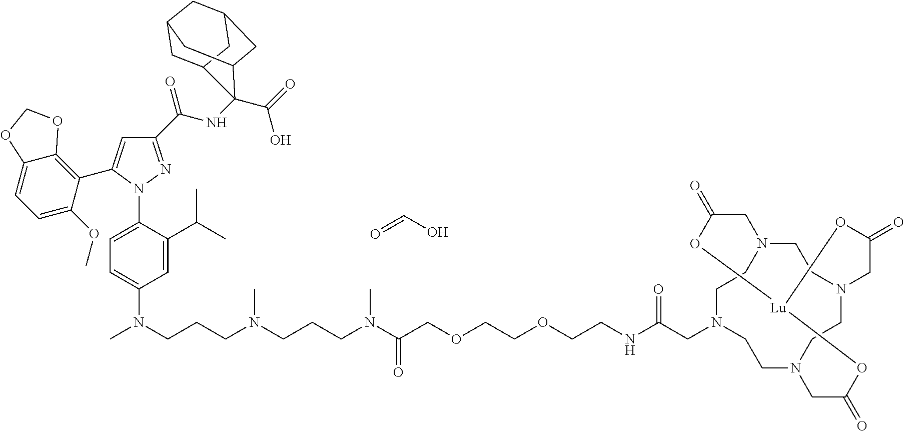 | 1433 [M + H]⁺ |
| 34-Lu | Lutetium(III) 2,2′,2″- (10-(2-((3-((4-((4-(3- (((5r,7r)-2- carboxyadamantan-2- yl)carbamoyl)-5-(5- methoxybenzo[d][1,3] dioxol-4-y1)-1H- pyrazol-1-yl)-3- isopropylphenyl) amino)-4-oxobutyl)((1- methylpyrrolidin-3- yl)methyl)amino) propyl)(methyl) amino)-2- oxoethyl)-1,4,7,10- tetraazacyclododecane- 1,4,7-triyl)triacetic acid; formic acid | 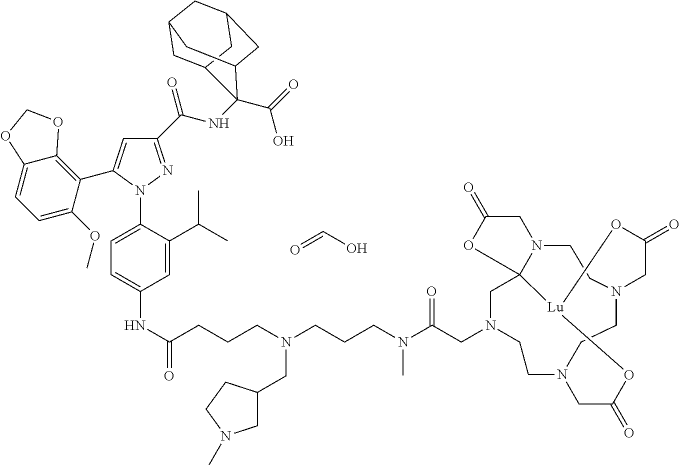 | 1385 [M + H]⁺ |
| 35-Lu | Lutetium(III) 2,2′,2″- (10-(2-((3-((1R,4R)-5- (4-(3-(((5R,7R)-2- Carboxyadamantan-2- yl)carbamoyl)-5-(5- methoxybenzo[d][1,3] dioxol-4-y1)-1H- pyrazol-1-yl)-3- isopropylphenyl)-2,5- diazabicyclo[2.2.1] heptan-2- yl)propyl)amino)-2- oxoethyl)-1,4,7,10- tetraazacyclododecane- 1,4,7-triyl)triacetic acid | 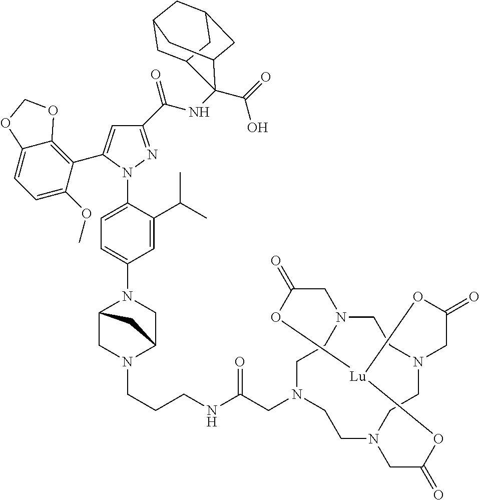 | 1270 [M + H]⁺ |
| 36-Lu | Lutetium(III) 2,2′,2″- (10-(2-((3-((1S,4S)-5- (4-(3-(((5S,7S)-2- Carboxyadamantan-2- yl)carbamoyl)-5-(5- methoxybenzo[d][1,3] dioxol-4-y1)-1H- pyrazol-1-y1)-3- isopropylphenyl)-2,5- diazabicyclo[2.2.1] heptan-2- yl)propyl)amino)-2- oxoethyl)-1,4,7,10- tetraazacyclododecane- 1,4,7-triyl)triacetic acid | 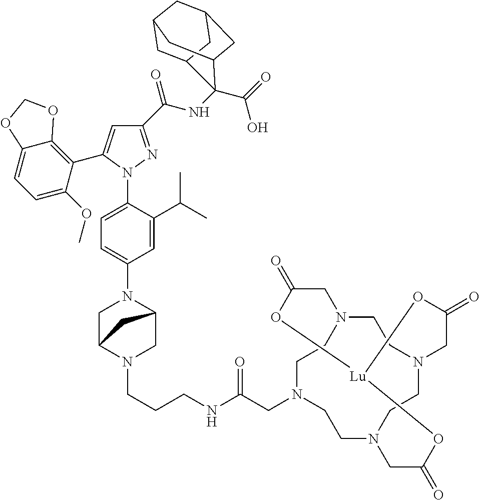 | 1270 [M + H]⁺ |
| 37-Lu | Lutetium(III) 2,2′,2″- (10-(2-((3-((3-(6-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl) quinoxaline-2- carboxamido)propyl) (methyl)amino)propyl) amino)-2-oxoethyl)- 1,4,7,10- tetraazacyclododecane- 1,4,7-triyl)triacetic acid | 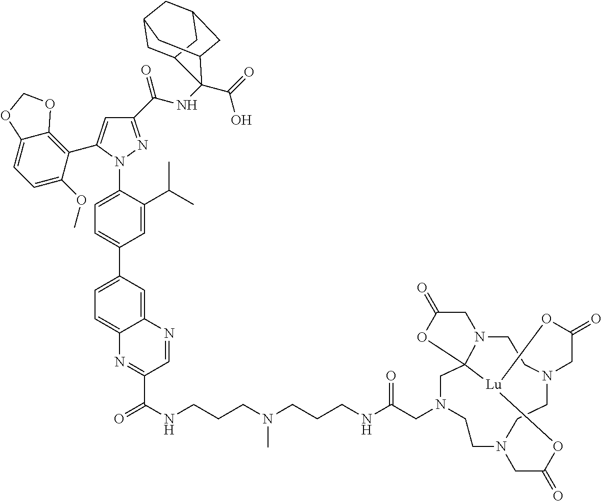 | 1416 [M + H]⁺ |
| 38-Lu | Lutetium(III) 2,2′,2″- (10-(2-((3-((3-(7-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl) quinoline-3- carboxamido)propyl) (methyl)amino)propyl) amino)-2-oxoethyl)- 1,4,7,10- tetraazacyclododecane- 1,4,7-triyl)triacetic acid | 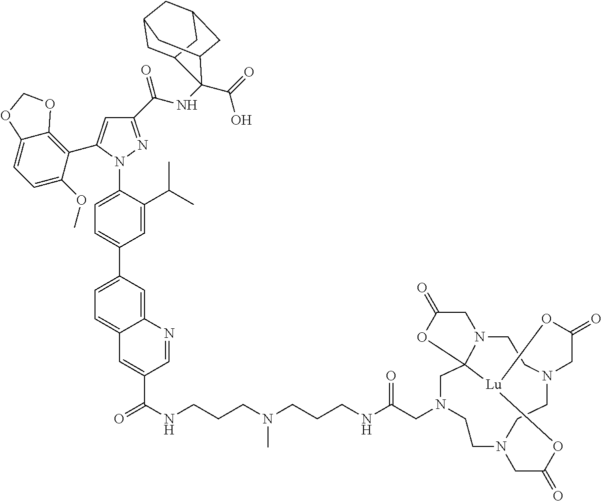 | 1415 [M + H]⁺ |
| 39-Lu | Lutetium(III) 2,2′,2″- (10-(2-((3-(7-(4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl)-2,7- diazaspiro[3.5]nonan- 2-yl)propyl)amino)-2- oxoethyl)-1,4,7,10- tetraazacyclododecane- 1,4,7-triyl)triacetic acid | 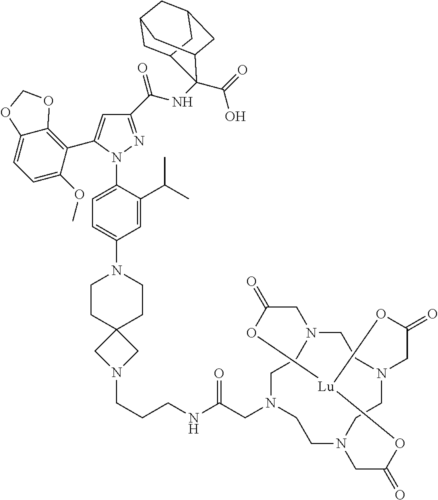 | 1298 [M + H]⁺ |
| 40-Lu | Lutetium(III) 2,2′,2″- (10-(2-((3-(2-(4-(3- (((5r,7r)-2- Carboxyadamantan-2- yl)carbamoy1)-5-(5- methoxybenzo[d][1,3] dioxol-4-y1)-1H- pyrazol-1-yl)-3- isopropylphenyl)-2,7- diazaspiro[3.5]nonan- 7-yl)propyl)amino)-2- oxoethyl)-1,4,7,10- tetraazacyclododecane- 1,4,7-triyl)triacetic acid | 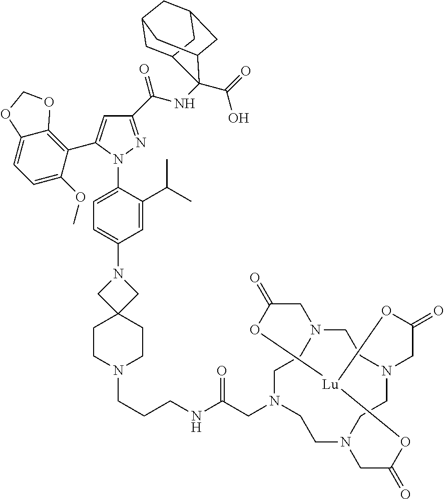 | 1298 [M + H]⁺ |
| 41-Lu | Lutetium(III) 2,2′,2″- (10-(2-((3-((3-(4-(4- (3-(((5r,7r)-2- Carboxyadamantan-2- yl)carbamoyl)-5-(5- methoxybenzo[d][1,3] dioxol-4-y1)-1H- pyrazol-1-y1)-3- isopropylphenyl) piperazin-1- yl)propyl)(methyl) amino)propyl)amino)- 2-oxoethyl)-1,4,7,10- tetraazacyclododecane- 1,4,7-triyl)triacetic acid | 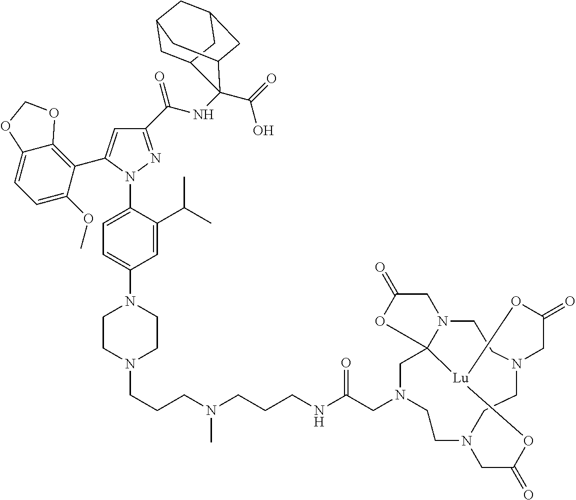 | 1329 [M + H]⁺ |
| 42-Lu | Lutetium(III) 2,2′,2″- (4-(2-((3-((3-((3-(4-(3- (((5r,7r)-2- Carboxyadamantan-2- yl)carbamoyl)-5-(5- methoxybenzo[d][1,3] dioxol-4-yl)-1H- pyrazol-1-y1)-3- isopropylphenyl) quinolin-7- yl)oxy)propyl)(methyl) amino)propyl)amino)- 2-oxoethyl)-1,4,7- triazacyclododecane- 1,7,10-triyl)triacetic acid | 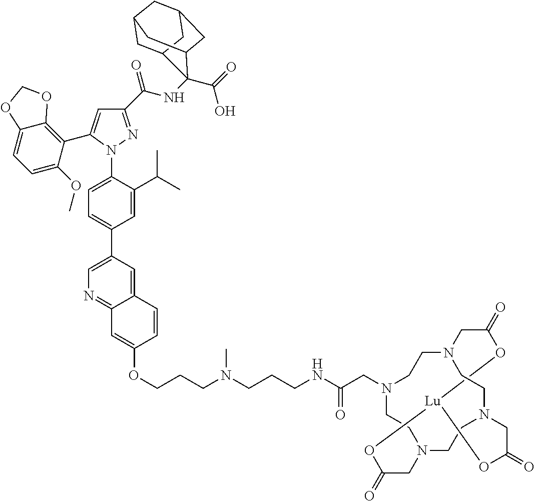 | 1388 [M + H]⁺ |
| 43-Lu | Lutetium(III) 2,2′,2″- (10-(2-((3-((3-(7-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl)-1,5- naphthyridine-3- carboxamido)propyl) (methyl)amino)propyl) amino)-2-oxoethyl)- 1,4,7,10- tetraazacyclododecane- 1,4,7-triyl)triacetic acid | 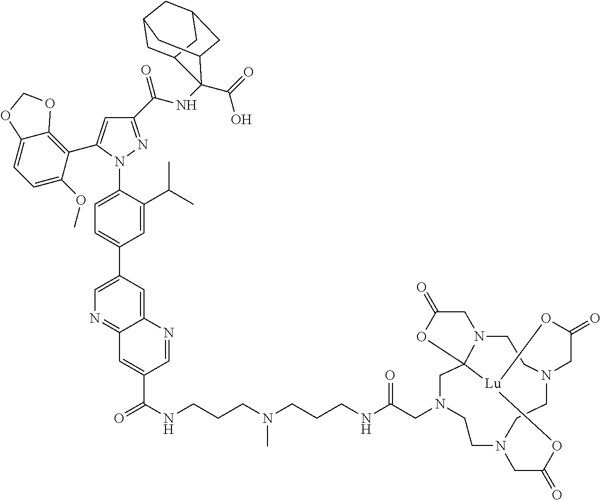 | 1416 [M + H]⁺ |
| 44-Lu | Lutetium(III) 2,2′,2″- (10-(2-((3-((3-(5-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl)-3- fluoropicolinamido) propyl)(methyl)amino) propyl)amino)-2- oxoethyl)-1,4,7,10- tetraazacyclododecane- 1,4,7-triyl)triacetic acid | 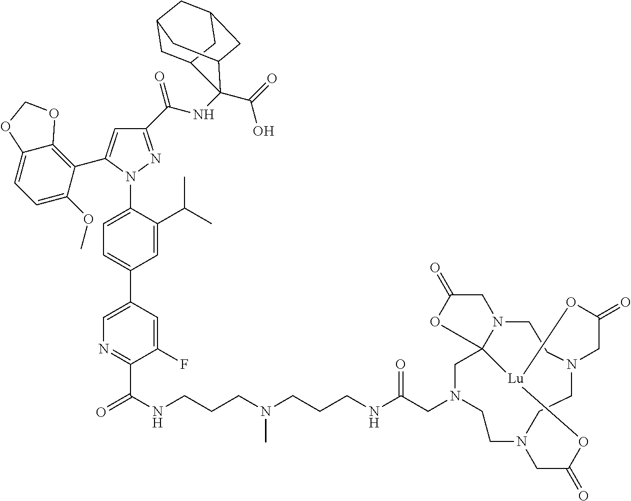 | 1382 [M + H]⁺ |
| 45-Lu | Lutetium(III) 2,2′,2″- (10-(1-((4-(3-(((5r,7r)- 2-Carboxyadamantan- 2-yl)carbamoyl)-5-(5- methoxybenzo[d][1,3] dioxol-4-y1)-1H- pyrazol-1-y1)-3- isopropylphenyl) amino)-13-methyl- 1,18-dioxo-3,6-dioxa- 9,13,17- triazanonadecan-19- yl)-1,4,7,10- tetraazacyclododecane- 1,4,7-triyl)triacetic acid | 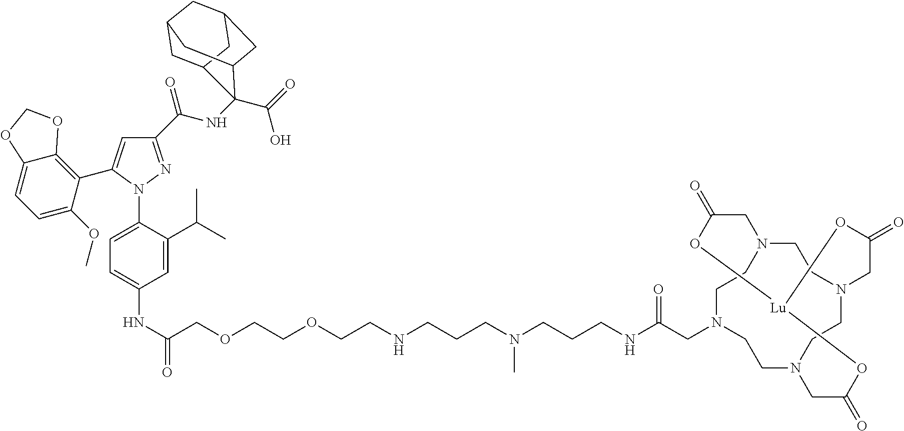 | 1405 [M + H]⁺ |
| 46-Lu | Lutetium(III) 2,2′,2″- (10-(2-((3-(9-(4-(3- (((5r,7r)-2- Carboxyadamantan-2- yl)carbamoyl)-5-(5- methoxybenzo[d][1,3] dioxol-4-y1)-1H- pyrazol-1-y1)-3- isopropylphenyl)-3,9- diazaspiro[5.5] undecan-3- yl)propyl)amino)- 2-oxoethyl)-1,4,7,10- tetraazacyclododecane- 1,4,7-triyl)triacetic acid | 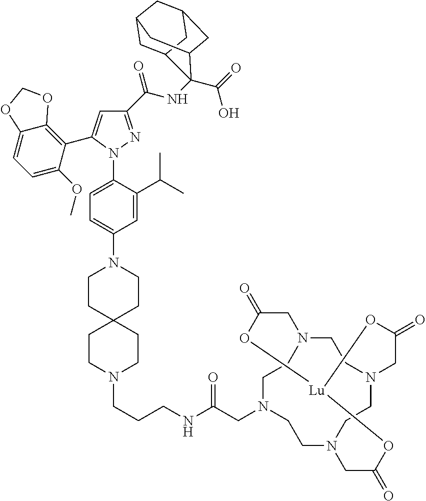 | 1326 [M + H]⁺ |
| 47-Lu | Lutetium(III) 2,2′,2″- (10-(2-(4-((1-((4-(3- (((5r,7r)-2- Carboxyadamantan-2- yl)carbamoyl)-5-(5- methoxybenzo[d][1,3] dioxol-4-yl)-1H- pyrazol-1-y1)-3- isopropylphenyl) carbamoyl)piperidin- 4-yl)methyl)piperazin- 1-yl)-2-oxoethyl)- 1,4,7,10- tetraazacyclododecane- 1,4,7-triyl)triacetic acid | 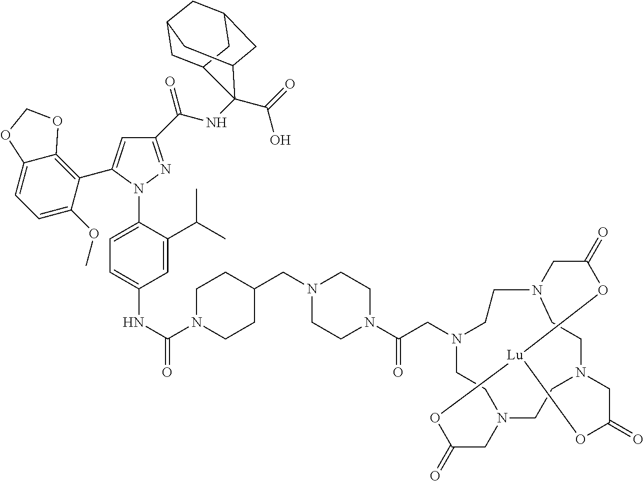 | 1341 [M + H]⁺ |
| 48-Lu | Lutetium(III) 2,2′,2″- (10-(2-((3-((3-(4′-(3- (((5r,7r)-2- Carboxyadamantan-2- yl)carbamoyl)-5-(5- methoxybenzo[d][1,3] dioxol-4-yl)-1H- pyrazol-1-yl)-2,6- difluoro-3′-isopropyl- [1,1′-biphenyl]-4- carboxamido)propyl) (methyl)amino)propyl) amino)-2-oxoethyl)- 1,4,7,10- tetraazacyclododecane- 1,4,7-triyl)triacetic acid | 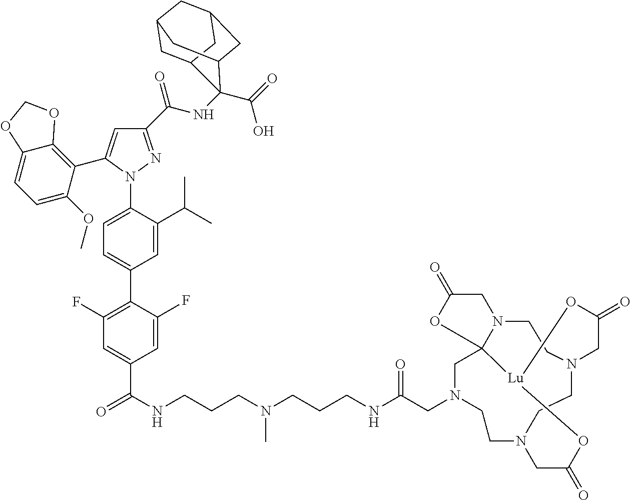 | 1400 [M + H]⁺ |
| 49-Lu | Lutetium(III) 2,2′,2″- (10-(2-((3-((3-(4′-(3- (((5r,7r)-2- Carboxyadamantan-2- yl)carbamoyl)-5-(5- methoxybenzo[d][1,3] dioxol-4-yl)-1H- pyrazol-1-yl)-3′- isopropyl-3-methoxy- [1,1′-biphenyl]-4- carboxamido)propyl) (methyl)amino)propyl) amino)-2-oxoethyl)- 1,4,7,10- tetraazacyclododecane- 1,4,7-triyl)triacetic acid | 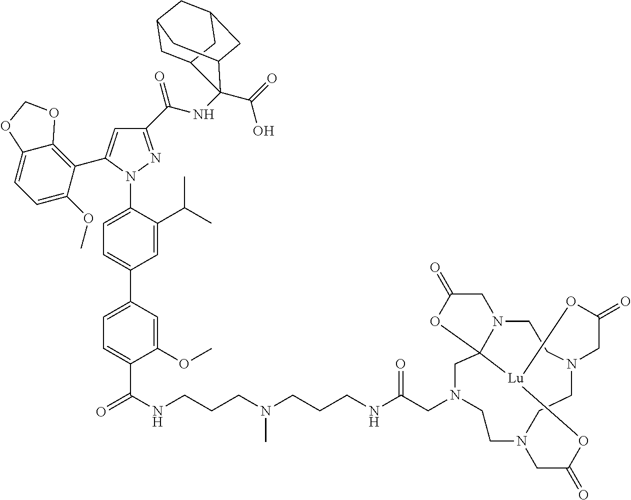 | 1394 [M + H]⁺ |
| 50-Lu | Lutetium(III) 2,2′,2″- (10-(2-((3-(4-(3-((4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl) amino)propyl) piperazin-1- yl)propyl)amino)-2- oxoethyl)-1,4,7,10- tetraazacyclododecane- 1,4,7-triyl)triacetic acid | 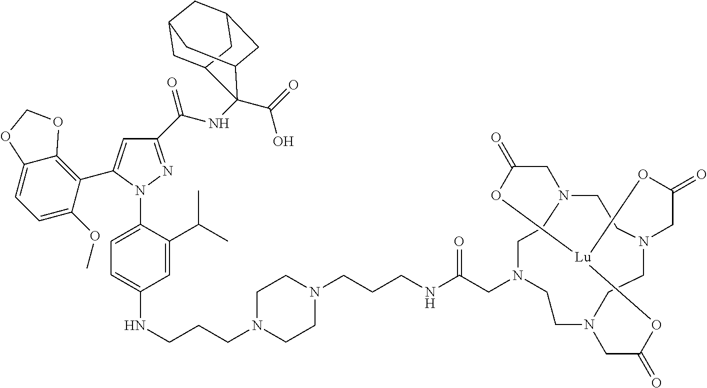 | 1315 [M + H]⁺ |
| 51-Lu | Lutetium(III) 2,2′,2″- (10-(2-(4-(2-(4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenoxy) ethyl)piperazin-1-yl)-2- oxoethyl)-1,4,7,10- tetraazacyclododecane- 1,4,7-triyl)triacetic acid | 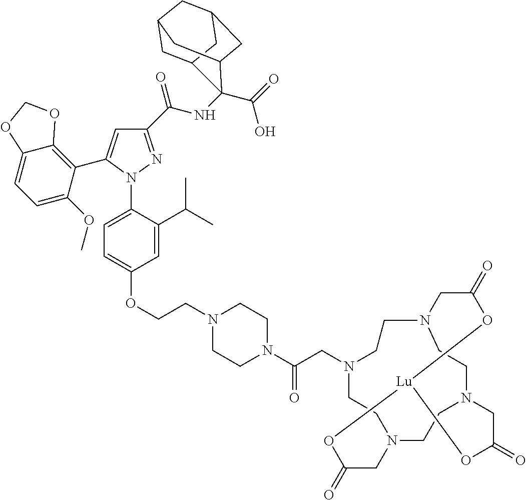 | 1245 [M + H]⁺ |
| 52-Lu | Lutetium(III) 2,2′,2″- (10-(2-((3-((4-((3-((4- (3-(((5r,7r)-2- Carboxyadamantan-2- yl)carbamoyl)-5-(5- methoxybenzo[d][1,3] dioxol-4-yl)-1H- pyrazol-1-yl)-3- isopropylphenyl) amino)propyl) (methyl)amino)butyl) (methyl)amino) propyl)amino)-2- oxoethyl)-1,4,7,10- tetraazacyclododecane- 1,4,7-triyl)triacetic acid | 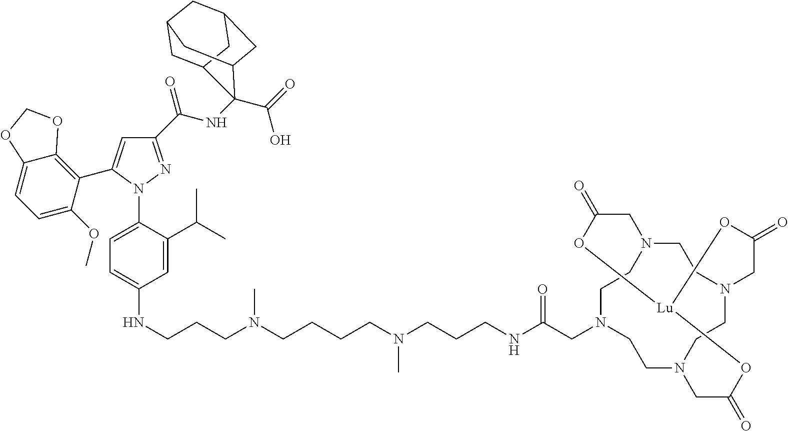 | 1345 [M + H]⁺ |
| 53-Lu | Lutetium(III) 2,2′,2″- (10-(2-((3-(3-(4-(3- (((5r,7r)-2- Carboxyadamantan-2- yl)carbamoy1)-5-(5- methoxybenzo[d][1,3] dioxol-4-y1)-1H- pyrazol-1-y1)-3- isopropylphenoxy) propoxy)propyl) amino)-2-oxoethyl)- 1,4,7,10- tetraazacyclododecane- 1,4,7-triyl)triacetic acid | 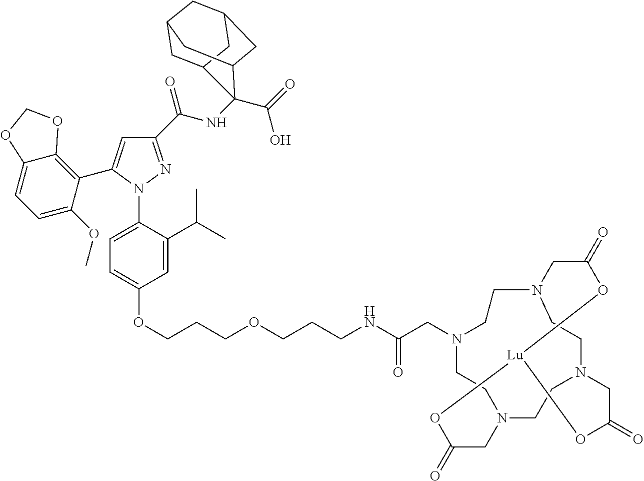 | 1248 [M + H]⁺ |
| 54-Lu | Lutetium(III) 2,2′,2″- (10-(2-(((2S)-4- Carboxy-1-(4-(2-(4- (3-(((5r,7r)-2- carboxyadamantan-2- yl)carbamoyl)-5-(5- methoxybenzo[d][1,3] dioxol-4-yl)-1H- pyrazol-1-yl)-3- isopropylphenoxy) ethyl)piperidin-1-yl)-1- oxobutan-2-yl)amino)- 2-oxoethyl)-1,4,7,10- tetraazacyclododecane- 1,4,7-triyl)triacetic acid | 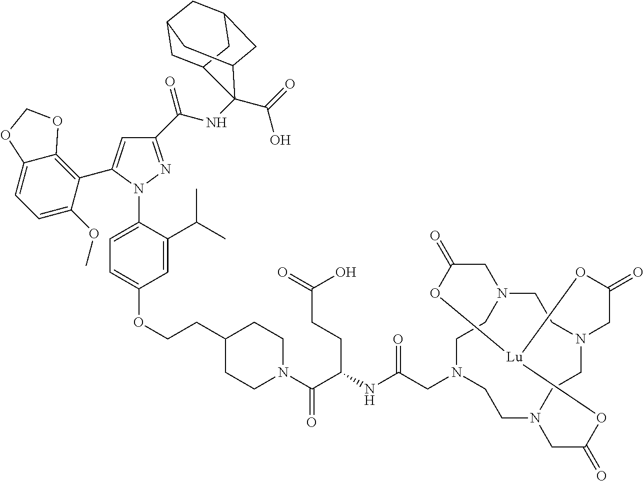 | 1373 [M + H]⁺ |
| 55-Lu | Lutetium(III) 2,2′,2″- (10-(2-(4-(3-(4-(3- (((5r,7r)-2- Carboxyadamantan-2- y1)carbamoyl)-5-(5- methoxybenzo[d][1,3] dioxol-4-y1)-1H- pyrazol-1-yl)-3- isopropylphenoxy) propyl)piperazin-1- y1)-2-oxoethyl)- 1,4,7,10- tetraazacyclododecane- 1,4,7-triyl)triacetic acid | 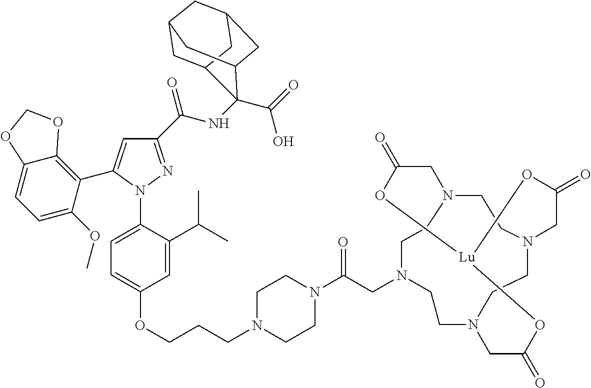 | 1259 [M + H]⁺ |
| 56-Lu | Lutetium(III) 2,2′,2″- (10-(2-((2-((1-((4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl) carbamoyl)piperidin- 4-yl)oxy)ethyl)amino)- 2-oxoethyl)-1,4,7,10- tetraazacyclododecane- 1,4,7-triyl)triacetic acid | 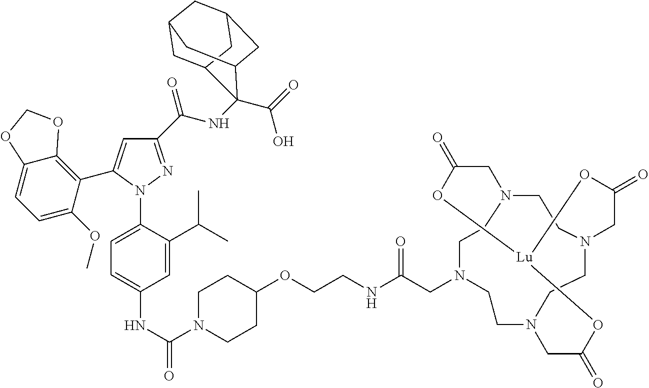 | 1302 [M + H]⁺ |
| 57-Lu | Lutetium(III) 2,2′,2″- (10-(2-((3-((3-((4′-(3- (((5r,7r)-2- Carboxyadamantan-2- yl)carbamoyl)-5-(5- methoxybenzo[d][1,3] dioxol-4-y1)-1H- pyrazol-1-yl)-3′- isopropyl-[1,1′- biphenyl]-4- yl)oxy)propyl)(methyl )amino)propyl)amino)- 2-oxoethyl)-1,4,7,10- tetraazacyclododecane- 1,4,7-triyl)triacetic acid | 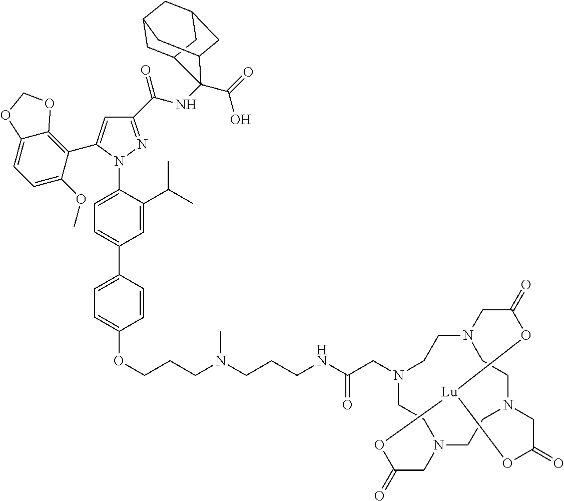 | 1337 [M + H]⁺ |
| 58-Lu | Lutetium(III) 2,2′,2″- (10-(2-((3-(9-(4-(3- (((5r,7r)-2- Carboxyadamantan-2- yl)carbamoyl)-5-(5- methoxybenzo[d][1,3] dioxol-4-yl)-1H- pyrazol-1-yl)-3- isopropylphenyl)-8- oxo-3,9- diazaspiro[5.5] undecan-3-yl)propyl) amino)-2-oxoethyl)- 1,4,7,10- tetraazacyclododecane- 1,4,7-triyl)triacetic acid | 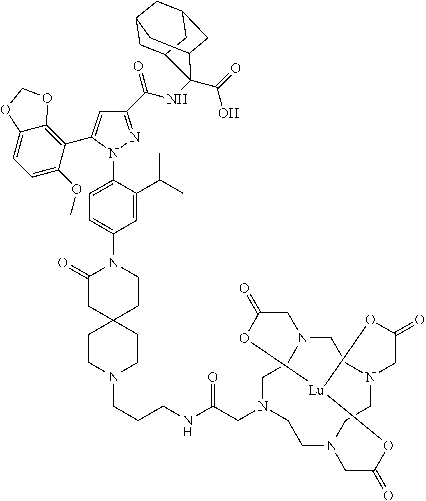 | 1340 [M + H]⁺ |
| 59-Lu | Lutetium(III) 2,2′,2″- (10-(1-Carboxy-4-((3- ((3-(3-(4-(3-(((5r,7r)- 2-carboxyadamantan- 2-yl)carbamoyl)-5-(5- methoxybenzo[d][1,3] dioxol-4-y1)-1H- pyrazol-1-yl)-3- isopropylphenyl) ureido)propyl) (methyl)amino) propyl)amino)-4- oxobutyl)-1,4,7,10- tetraazacyclododecane- 1,4,7-triyl)triacetic acid | 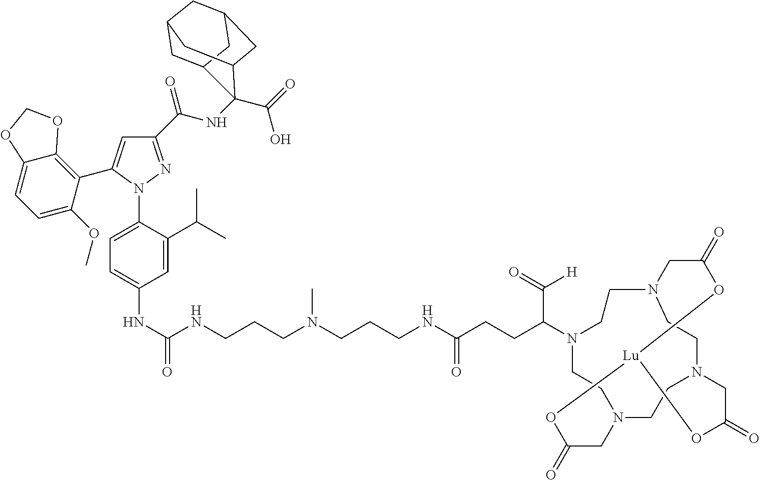 | 1376 [M + H]⁺ |
| 60-Lu | Lutetium(III) 2,2′,2″- (10-(2-((3-((3-(5-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl)- 1,3,4-oxadiazol-2- yl)propyl)(methyl) amino)propyl)amino)- 2-oxoethyl)-1,4,7,10- tetraazacyclododecane- 1,4,7-triyl)triacetic acid | 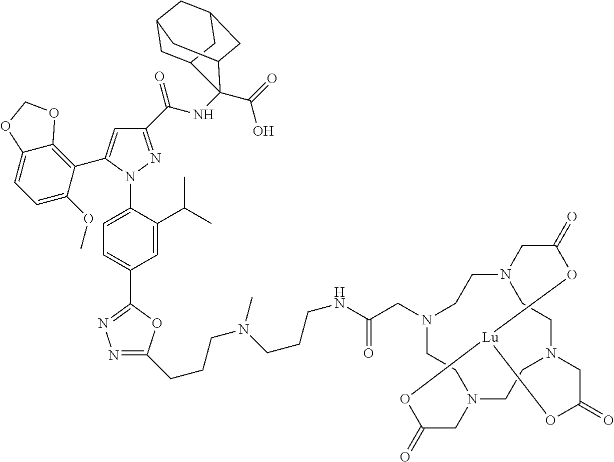 | 1314 [M + H]⁺ |
| 61-Lu | Lutetium(III) 2,2′,2″- (10-(2-((3-((3-(4-(3- (((5r,7r)-2- Carboxyadamantan-2- yl)carbamoyl)-5-(5- cyanobenzo[d][1,3] dioxol-4-y1)-1H- pyrazol-1-yl)-3- isopropylphenoxy) propyl)(methyl) amino)propyl)amino)- 2-oxoethyl)-1,4,7,10- tetraazacyclododecane- 1,4,7-triyl)triacetic acid | 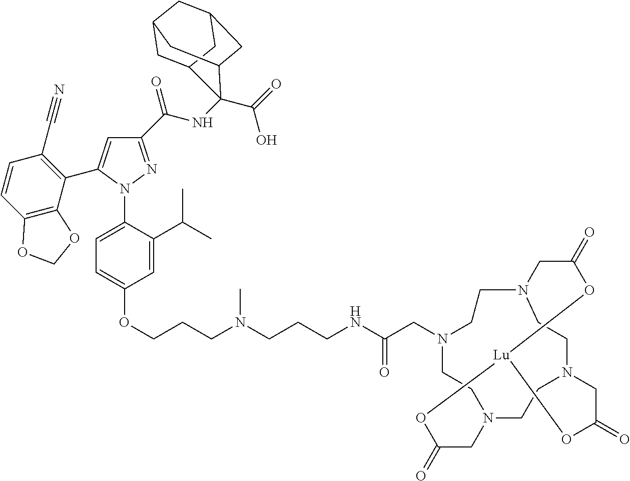 | 1254 [M + H]⁺ |
| 62-Lu | Lutetium(III) 2,2′,2″- (10-(2-((3-(4-(2-(4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenoxy) ethyl)piperazin-1- y1)propyl)amino)-2- oxoethyl)-1,4,7,10- tetraazacyclododecane- 1,4,7-triyl)triacetic acid | 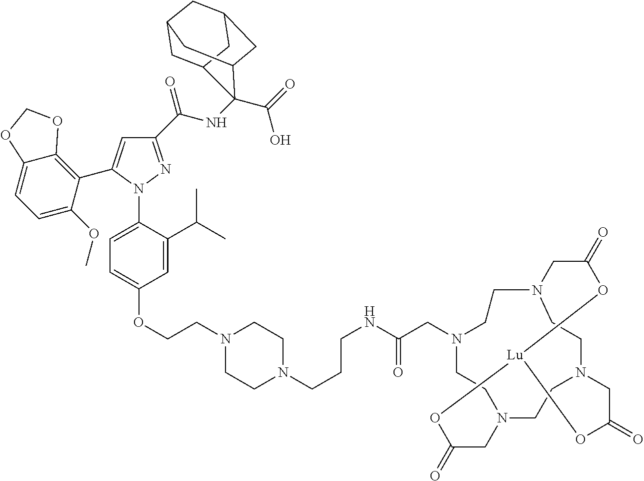 | 1303 [M + H]⁺ |
| 63-Lu | Lutetium(III) 2,2′,2″- (10-(2-((3-((3-(7-(4- (3-(((5r,7r)-2- Carboxyadamantan-2- yl)carbamoyl)-5-(5- methoxybenzo[d][1,3] dioxol-4-yl)-1H- pyrazol-1-y1)-3- isopropylphenyl)- [1,2,3]triazolo[1,5- a]pyridine-3- carboxamido)propyl) methyl)amino)propyl) amino)-2-oxoethyl)- 1,4,7,10- tetraazacyclododecane- 1,4,7-triyl)triacetic acid | 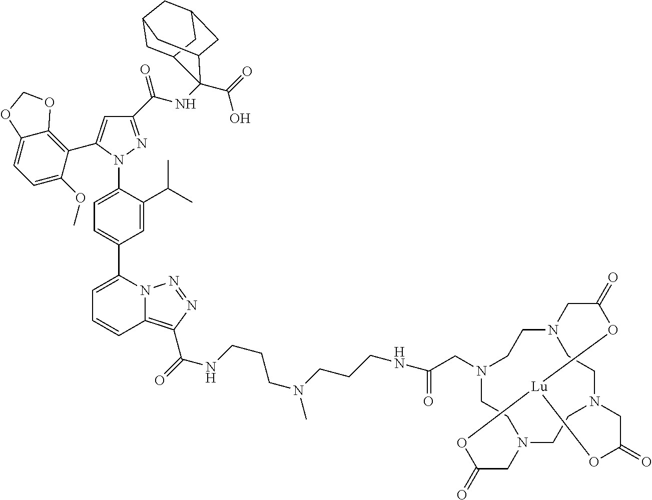 | 1405 [M + H]⁺ |
| 64-Lu | Lutetium(III) 2,2′,2″- (10-(2-((3-((3-(3-(6- (3-(((5r,7r)-2- Carboxyadamantan-2- yl)carbamoyl)-5-(5- methoxybenzo[d][1,3] dioxol-4-y1)-1H- pyrazol-1-yl)-5- isopropylpyridin-3- yl)ureido)propyl) (methyl)amino) propyl)amino)-2- oxoethyl)-1,4,7,10- tetraazacyclododecane- 1,4,7-triyl)triacetic acid | 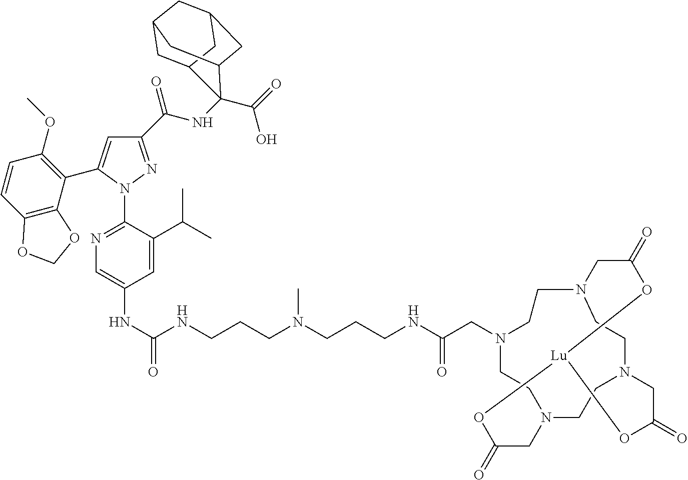 | 1304 [M + H]⁺ |
| 65-Lu | Lutetium(III) 2,2′,2″- (10-(2-((3-(3-((4-(3- (((5r,7r)-2- Carboxyadamantan-2- yl)carbamoyl)-5-(5- methoxybenzo[d][1,3] dioxol-4-y1)-1H- pyrazol-1-y1)-3- isopropylphenyl) amino)propoxy) propyl)amino)-2- oxoethyl)-1,4,7,10- tetraazacyclododecane- 1,4,7-triyl)triacetic acid | 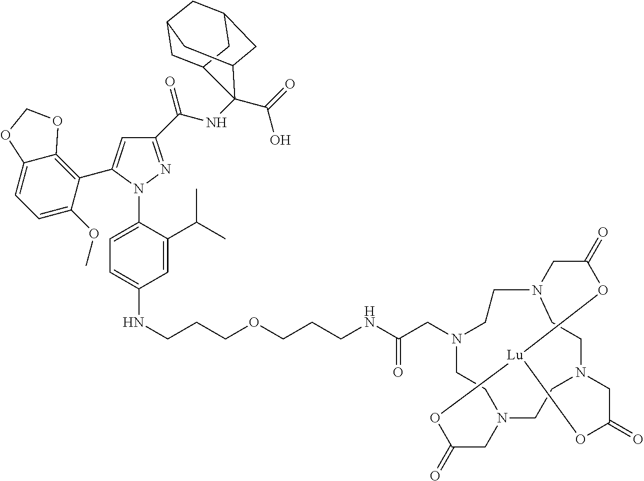 | 624 [M/2]⁺ |
| 66-Lu | Lutetium(III) 2,2′,2″- (10-(2-((3-((3-((4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl) amino)-2,2- bis(hydroxymethyl) propyl)amino)propyl) amino)-2-oxoethyl)- 1,4,7,10- tetraazacyclododecane- 1,4,7-triyl)triacetic acid | 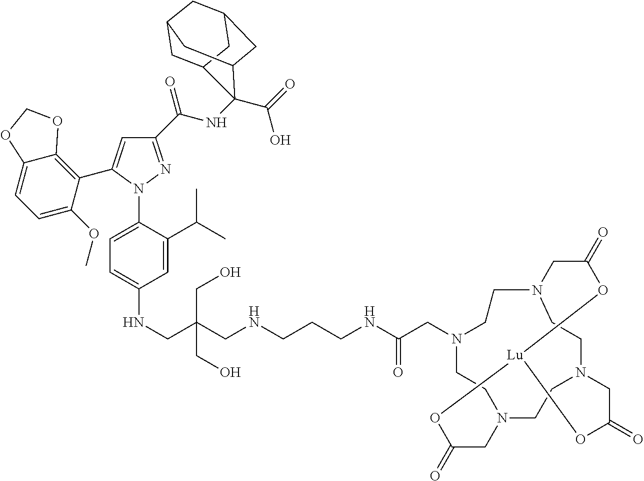 | 1306 [M + H]⁺ |
| 67-Lu | Lutetium(III) 2,2′,2″- (10-(2-((3-(3-(((4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl) amino)methyl)-3- (hydroxymethyl) azetidin-1-yl)propyl) amino)-2-oxoethyl)- 1,4,7,10- tetraazacyclododecane- 1,4,7-triyl)triacetic acid | 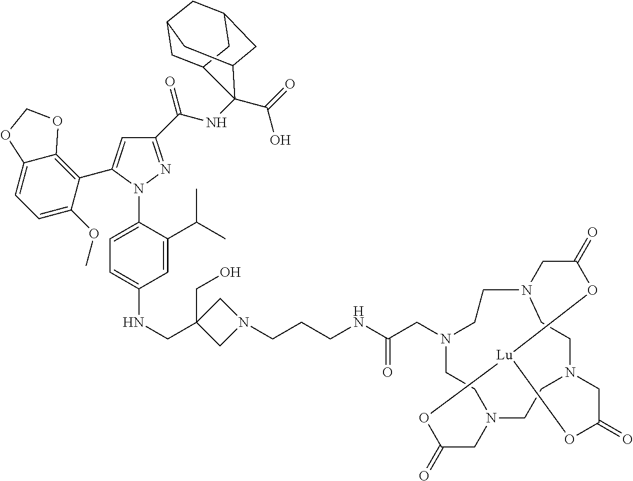 | 1306 [M + H]⁺ |
| 68-Lu | Lutetium(III) 2,2′,2″- (10-(2-((3-((3-(4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenoxy) propyl)(methyl)amino) propyl)amino)-2- oxoethyl)-1,4,7,10- tetraazacyclododecane- 1,4,7-triyl)triacetic acid | 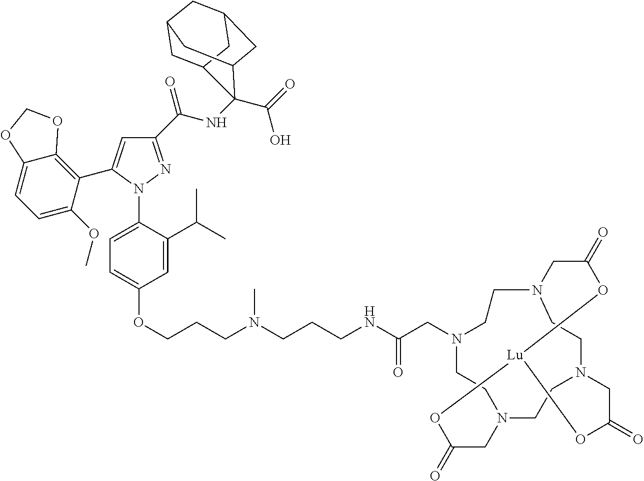 | 1261 [M + H]⁺ |
| 69-Lu | Lutetium(III) 2,2′,2″- (10-(2-((3-((3-(4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenoxy) propyl)(3- (dimethylamino) propy1)amino) propyl)amino)- 2-oxoethyl)-1,4,7,10- tetraazacyclododecane- 1,4,7-triyl)triacetic acid | 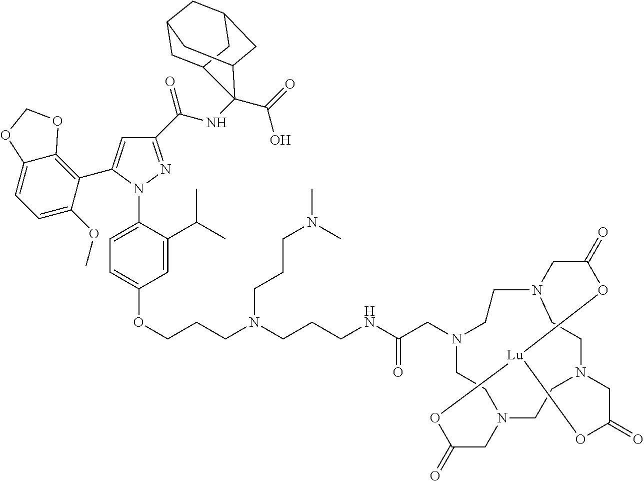 | 1332 [M + H]⁺ |
| 70-Lu | Lutetium(III) 2,2′,2″- (10-(2-((3-(6-((4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl) carbamoyl)-2,6- diazaspiro[3.3]heptan- 2-yl)propyl)amino)-2- oxoethyl)-1,4,7,10- tetraazacyclododecane- 1,4,7-triyl)triacetic acid | 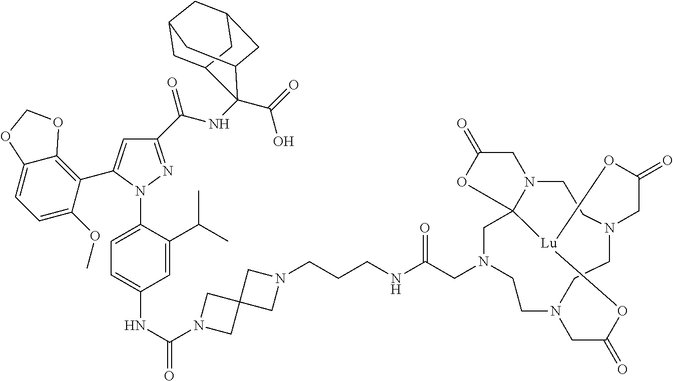 | 1313 [M + H]⁺ |
| 71-Lu | Lutetium(III) 2,2′,2″- (10-(2-((3-((3-(4-(3- (((S)-((3S,5S,7S)- Adamantan-1- yl)(carboxy)methyl) carbamoyl)-5-(5- methoxybenzo[d][1,3] dioxol-4-yl)-1H- pyrazol-1-yl)-3- isopropylphenoxy) propyl)(methyl)amino) propyl)amino)-2- oxoethyl)-1,4,7,10- tetraazacyclododecane- 1,4,7-triyl)triacetic acid | 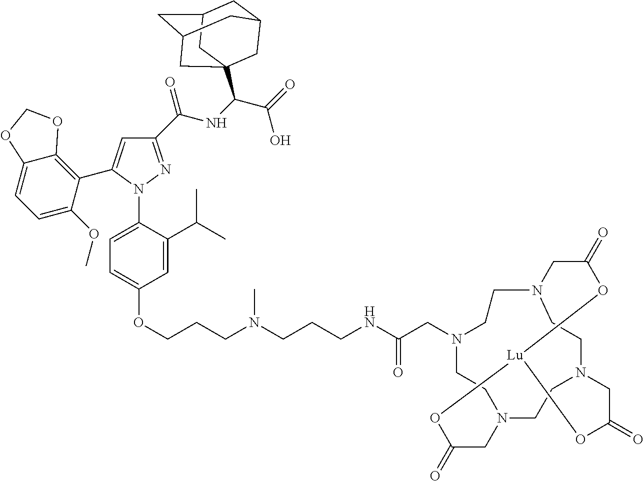 | 1275 [M + H]⁺ |
| 72-Lu | Lutetium(III) 2,2′,2″- (10-(2-((3-((3-((4-(3- (((S)-((3S,5S,7S)- Adamantan-1- yl)(carboxy)methyl) carbamoyl)-5-(5- methoxybenzo[d][1,3] dioxol-4-y1)-1H- pyrazol-1-y1)-3- isopropylphenyl) amino)propyl) (methyl)amino) propyl)amino)-2- oxoethyl)-1,4,7,10- tetraazacyclododecane- 1,4,7-triyl)triacetic acid | 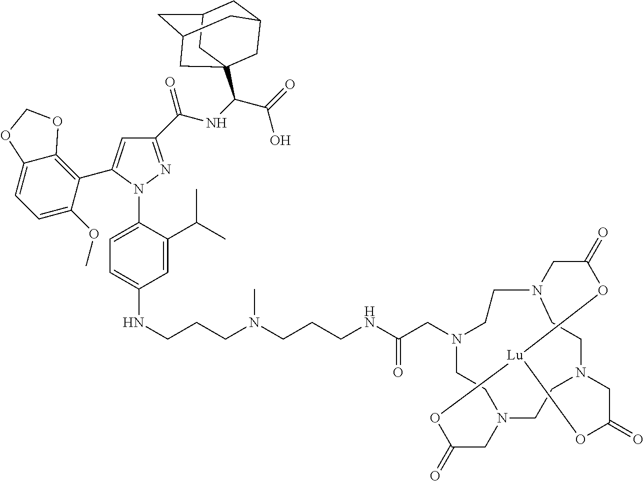 | 1274 [M + H]⁺ |
| 73-Lu | Lutetium(III) 2,2′,2″- (10-(2-((3-((3-(3-(4- (3-(((S)-((3S,5S,7S)- Adamantan-1- yl)(carboxy)methyl) carbamoyl)-5-(5- methoxybenzo[d][1,3] dioxol-4-yl)-1H- pyrazol-1-yl)-3- isopropylphenyl) ureido)propyl) (methyl)amino) propyl)amino)-2- oxoethyl)-1,4,7,10- tetraazacyclododecane- 1,4,7-triyl)triacetic acid | 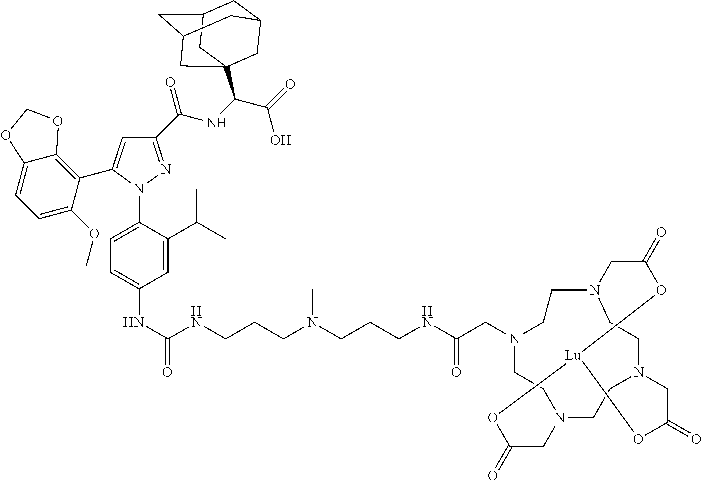 | 1317 [M + H]⁺ |
| 74-Lu | Lutetium(III) 2,2′,2″- (10-(2-((3-(4-((4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenoxy) methyl)piperidin-1- yl)propyl)amino)-2- oxoethyl)-1,4,7,10- tetraazacyclododecane- 1,4,7-triyl)triacetic acid | 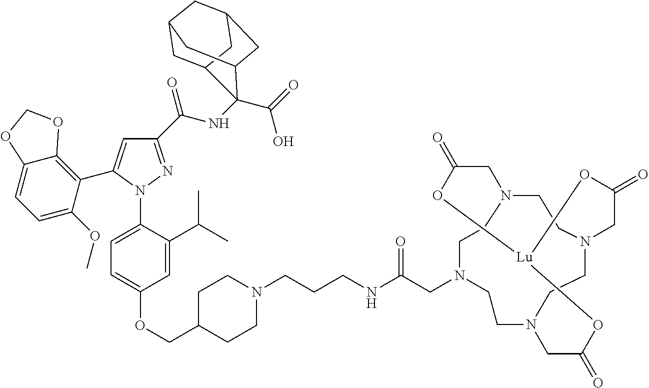 | 644 [M/2 + H]⁺ |
| 75-Lu | Lutetium(III) 2,2′,2″- (10-(2-(4-(3-(4-(2-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenoxy) ethyl)piperazin-1- yl)propyl)piperazin-1- yl)-2-oxoethyl)- 1,4,7,10- tetraazacyclododecane- 1,4,7-triyl)triacetic acid | 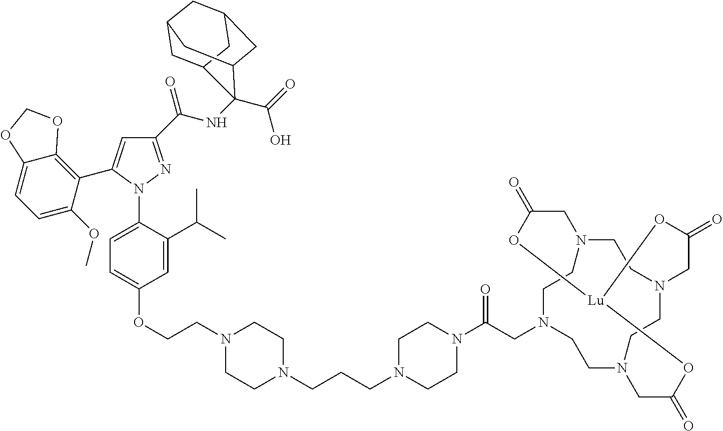 | 1370 [M + H]⁺ |
| 76-Lu | Lutetium(III) 2,2′,2″- (10-(2-((3-((3-(3-(4- (3-(((5r,7r)-2- Carboxyadamantan-2- yl)carbamoyl)-5-(5- methoxybenzo[d][1,3] dioxol-4-y1)-1H- pyrazol-1-y1)-3- isopropylphenyl)- [1,2,3]triazolo[1,5- a]pyridine-7- carboxamido)propyl) (methyl)amino)propyl) amino)-2-oxoethyl)- 1,4,7,10- tetraazacyclododecane- 1,4,7-triyl)triacetic acid | 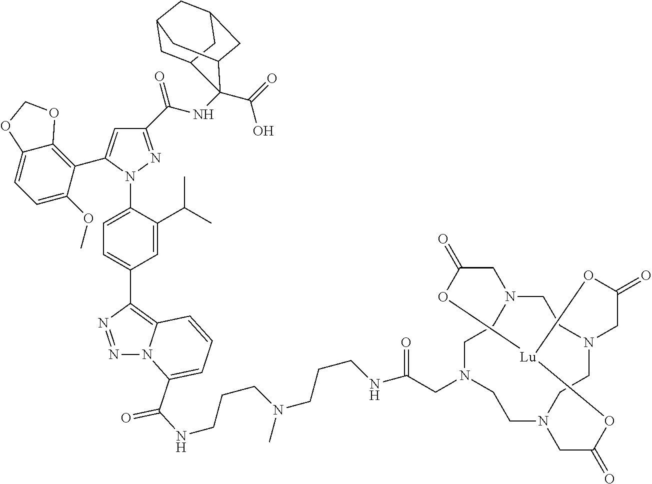 | 1403 [M + H]⁺ |
| 77-Lu | Lutetium(III) 2,2′,2″- (10-(2-((3-(6-(4-(3- (((5r,7r)-2- Carboxyadamantan-2- yl)carbamoyl)-5-(5- methoxybenzo[d][1,3] dioxol-4-y1)-1H- pyrazol-1-y1)-3- isopropylphenyl)-2,6- diazaspiro[3.3]heptan- 2-yl)propyl)amino)-2- oxoethyl)-1,4,7,10- tetraazacyclododecane- 1,4,7-triyl)triacetic acid | 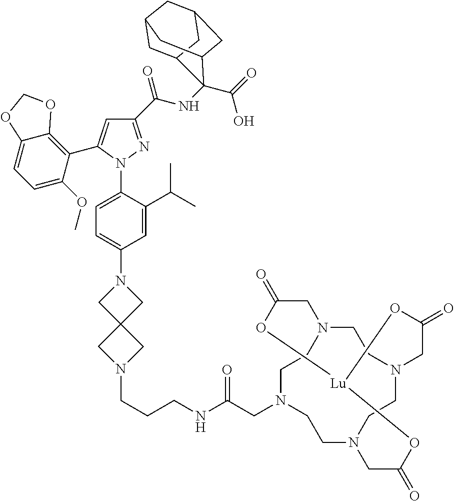 | 1270 [M + H]⁺ |
| 78-Lu | Lutetium(III) 2,2′,2″- (10-(2-((3-((4-((4-(3- (((5r,7r)-2- Carboxyadamantan-2- yl)carbamoyl)-5-(5- methoxybenzo[d][1,3] dioxol-4-yl)-1H- pyrazol-1-yl)-3- isopropylphenyl) amino)-4-oxobutyl)(3- (dimethylamino) propy1)amino)propyl) (methy1)amino)-2- oxoethyl)-1,4,7,10- tetraazacyclododecane- 1,4,7-triyl)triacetic acid | 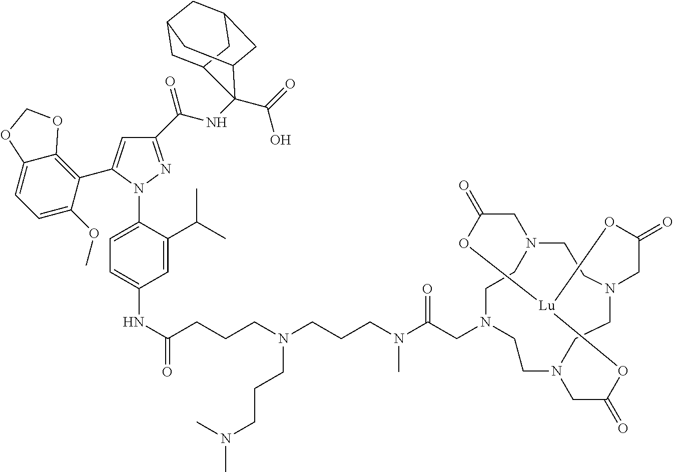 | 1359 [M + H]⁺ |
| 79-Lu | Lutetium(III) 2,2′,2″- (10-(2-((3-((3-((6-(3- (((5r,7r)-2- Carboxyadamantan-2- yl)carbamoyl)-5-(5- methoxybenzo[d][1,3] dioxol-4-y1)-1H- pyrazol-1-yl)-5- isopropylpyridin-3- yl)oxy)propyl) (methyl)amino) propyl)amino)- 2-oxoethyl)-1,4,7,10- tetraazacyclododecane- 1,4,7-triyl)triacetic acid | 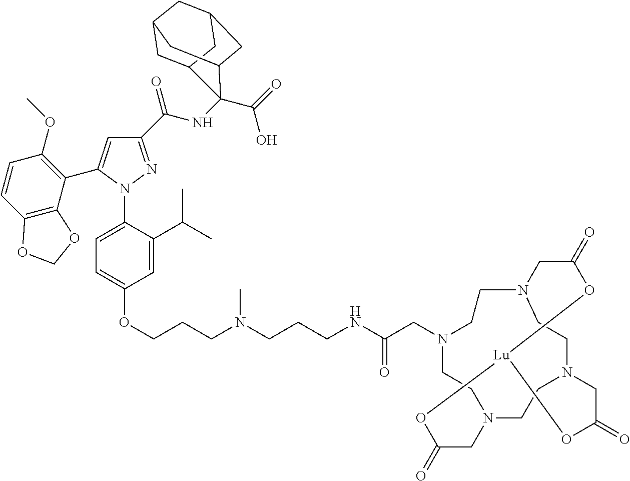 | 1262 [M + H]⁺ |
| 80-Lu | Lutetium(III) 2,2′,2″- (10-(2-((3-((3-((5-(3- (((5r,7r)-2- Carboxyadamantan-2- yl)carbamoyl)-5-(5- methoxybenzo[d][1,3] dioxol-4-yl)-1H- pyrazol-1-y1)-6- isopropylpyridin-2- yl)oxy)propyl) (methyl)amino)propyl) amino)-2-oxoethyl)- 1,4,7,10- tetraazacyclododecane- 1,4,7-triyl)triacetic acid | 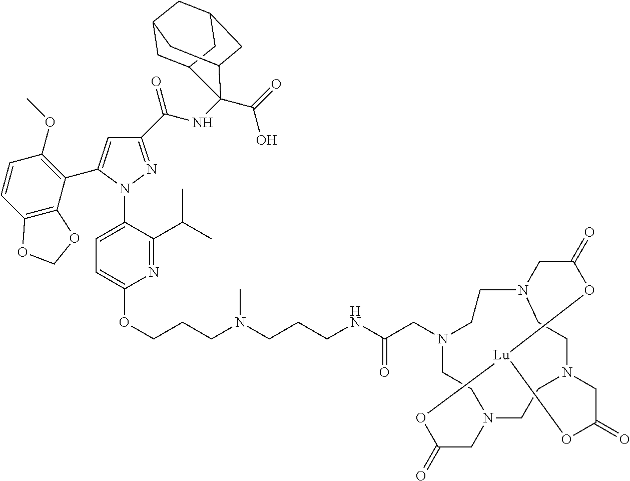 | 1262 [M + H]⁺ |
| 81-Lu | Lutetium(III) 2,2′,2″- (10-(2-((3-(3-(3-(4-(3- (((5r,7r)-2- Carboxyadamantan-2- yl)carbamoyl)-5-(5- methoxybenzo[d][1,3] dioxol-4-y1)-1H- pyrazol-1-yl)-3- isopropylphenyl) ureido)propoxy) propyl)amino)-2- oxoethyl)-1,4,7,10- tetraazacyclododecane- 1,4,7-triyl)triacetic acid | 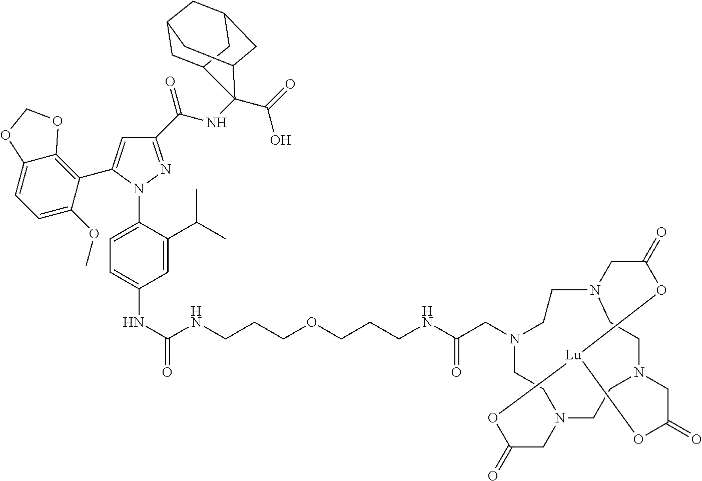 | 1290 [M + H]⁺ |
| 82-Lu | Lutetium(III) 2,2′,2″- (10-(2-((3-((3-(3-(5- (3-(((5r,7r)-2- Carboxyadamantan-2- yl)carbamoyl)-5-(5- methoxybenzo[d][1,3] dioxol-4-y1)-1H- pyrazol-1-yl)-6- isopropylpyridin-2- yl)ureido)propyl) (methyl)amino) propyl)amino)-2- oxoethyl)-1,4,7,10- tetraazacyclododecane- 1,4,7-triy1)triacetic acid | 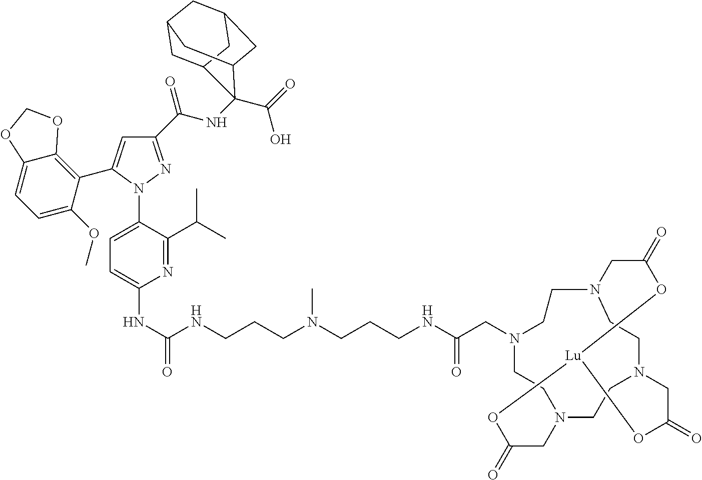 | 1304 [M + H]⁺ |
| 83-Lu | Lutetium(III) 2,2′,2″- (10-(2-(4-(3-((4-(3- (((5r,7r)-2- Carboxyadamantan-2- y1)carbamoyl)-5-(5- methoxybenzo[d][1,3] dioxol-4-y1)-1H- pyrazol-1-yl)-3- isopropylphenyl) amino)-3- oxopropyl)piperazin- 1-yl)-2-oxoethyl)- 1,4,7,10- tetraazacyclododecane- 1,4,7-triyl)triacetic acid | 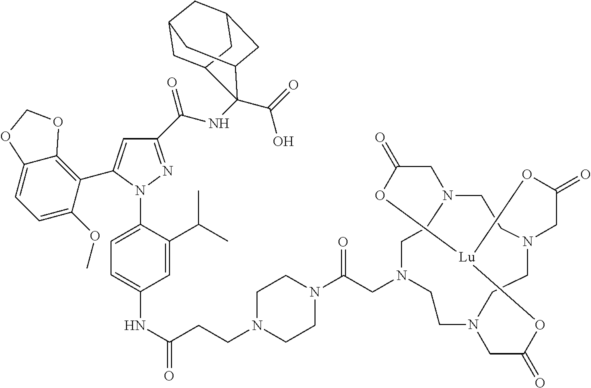 | 1272 [M + H]⁺ |
| 84-Lu | Lutetium(III) 2,2′,2″- (10-(2-(((1-(3-(3-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl) ureido)propyl)-1H- imidazol-4- yl)methyl)amino)-2- oxoethyl)-1,4,7,10- tetraazacyclododecane- 1,4,7-triyl)triacetic acid | 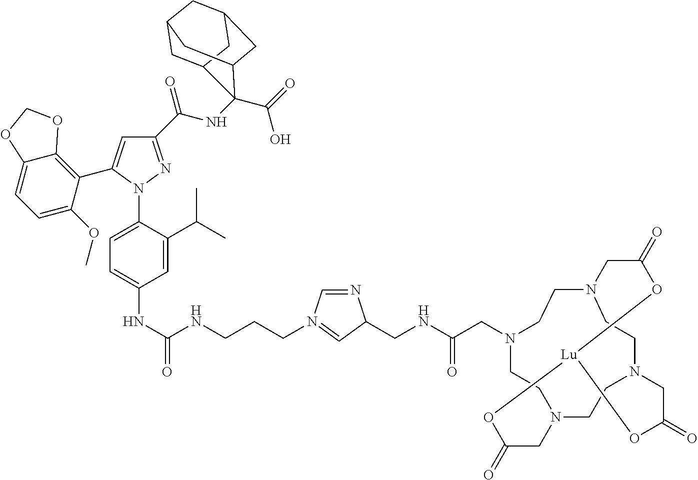 | 1312 [M + H]⁺ |
| 85-Lu | Lutetium(III) 2,2′,2″- (10-(2-(((1-(3-(3-(4- (3-(((5r,7r)-2- Carboxyadamantan-2- yl)carbamoyl)-5-(5- methoxybenzo[d][1,3] dioxol-4-y1)-1H- pyrazol-1-yl)-3- isopropylphenyl) ureido)propyl)-3- fluoroazetidin-3- yl)methyl)amino)-2- oxoethyl)-1,4,7,10- tetraazacyclododecane- 1,4,7-triyl)triacetic acid | 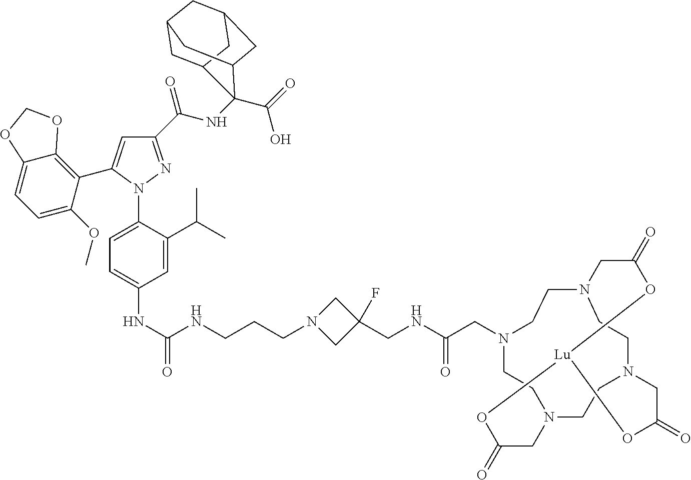 | 1320 [M + H]⁺ |
| 86-Lu | Lutetium(III) 2,2′,2″- (10-(2-((3-((3-(3-(4- (3-(((5r,7r)-2- Carboxyadamantan-2- yl)carbamoyl)-5-(5- cyanobenzo[d][1,3] dioxol-4-yl)-1H- pyrazol-1-y1)-3- isopropylphenyl) ureido)propyl) (methyl)amino) propyl)amino)-2- oxoethyl)-1,4,7,10- tetraazacyclododecane- 1,4,7-triyl)triacetic acid | 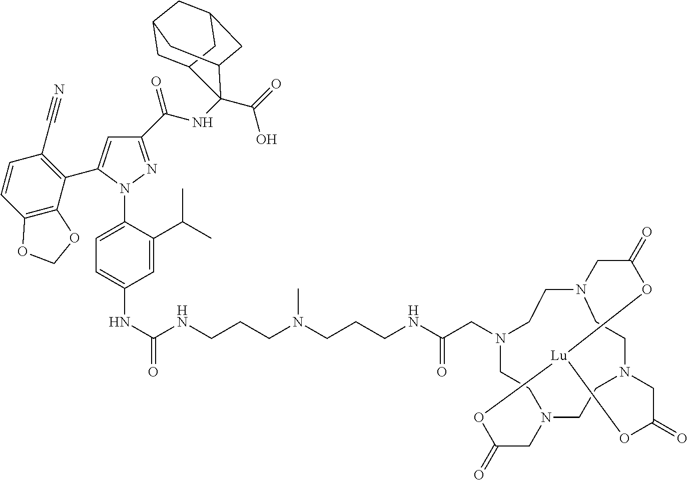 | 1298 [M + H]⁺ |

### NTS-Displacement Assay:

NTS-displacement assay is a cell binding assay to evaluate NTSR1 antagonist compounds for the binding affinity and functional activity of potential antagonists or RLTs. Activation of NTSR1 by neurotensin (NTS) induces internalization of NTSR1 and pre-treatment of cells with antagonist compounds blocks the binding of NTS to NTSR1 and subsequently NTSR1 internalization. Internalization of NTSR1 is measured by the loss of surface HiBiT-NTSR1 using a commercially available extracellular HiBiT detection kit purchased from Promega.

For NTS-displacement assay, HT29 HiBiT-NTSR1 clone #19 (CL-6759) was generated by knocking-in a HiBiT-tag at N-terminus domain of NTSR1 gene at genomic DNA level. Briefly, 10,000 HT29 HiBiT-NTSR1 Clone #19 cells (27 ul cell suspension) were plated in each well of 384-well plate and incubated for overnight in cell culture incubator at 37C. The next day, 3 ul of 10× concentrated compounds (diluted in media) were added to respective wells and incubated for 30 minutes in cell culture incubator at 37C followed by the addition of 3.3 ul of NTS (10 nM final concentration) for an additional 60 minutes. After incubation with NTS, 33 ul of extracellular detection reagent was added to each well for 3-5 minutes before reading the plate using Synergy Neo2 plate reader. The raw data was uploaded to genescreener to analyze NTS-displacement data.

Table 22 illustrates the IC₅₀ of neurotensin receptor ligands A-S in the NTS-displacement assay. For IC₅₀ values shown in Table 22, “A” means an IC₅₀ value of <10 nm; and B means an IC₅₀ value ranging between 10 nm and 50 nm; and C means an IC₅₀ value of >50 nm.

TABLE 22

| Ex # | NTS displacement Assay IC₅₀(nm) |
|---|---|
| A | A |
| B | A |
| C | A |
| D | A |
| E | A |
| F | A |
| G | A |
| H | B |
| I | A |
| J | A |
| K | A |
| L | A |
| M | A |
| N | A |
| O | A |
| P | A |
| Q | A |
| R | A |
| S | A |
| T | A |
| U | A |
| V | A |
| W | B |
| X | B |
| Y | A |
| Z | A |
| AA | C |
| AB | A |
| AC | C |
| AD | C |
| AE | A |
| AF | A |
| AG | A |
| AH | B |
| AI | B |
| AJ | B |
| AK | A |
| AL | A |
| AM | A |
| AN | C |
| AO | C |
| AP | A |
| AQ | A |
| AR | A |
| AS | A |
| AT | B |
| AU | A |
| AV | A |
| AW | A |
| AX | A |
| AY | C |
| AZ | C |
| BA | A |
| BB | B |
| BC | A |
| BD | A |
| BE | B |
| BF | A |
| BG | B |
| BH | C |
| BI | B |
| BJ | B |
| BK | C |
| BL | B |
| BM | C |
| BN | B |
| BO | B |
| BP | B |
| BQ | B |
| BR | B |

Table 23 illustrates the IC₅₀ of the compounds of Formula (I) in the NTS-displacement assay. For IC₅₀ values shown in Table 23, “A” means an IC₅₀ value of <10 nm; and B means an IC₅₀ value ranging between 10 nm and 50 nm.

TABLE 23

| Ex # | NTS displacement Assay IC₅₀(nm) |
|---|---|
| 10 | A |
| 1-Lu | A |
| 2-Lu | A |
| 3-Lu | A |
| 4-Lu | A |
| 5-Lu | B |
| 6-Lu | A |
| 7-Lu | A |
| 8-Lu | A |
| 9-Lu | A |
| 10-Lu | A |
| 11-Lu | A |
| 12-Lu | A |
| 13-Lu | A |
| 14-Lu | A |
| 15-Lu | A |
| 16-Lu | A |
| 17-Lu | A |
| 18-Lu | A |
| 19-Lu | A |
| 20-Lu | A |
| 21-Lu | A |
| 22-Lu | A |
| 23-Lu | A |
| 24-Lu | A |
| 25-Lu | B |
| 26-Lu | A |
| 27-Lu | A |
| 28-Lu | A |
| 29-Lu | A |
| 30-Lu | A |
| 31-Lu | A |
| 32-Lu | A |
| 33-Lu | A |
| 34-Lu | A |
| 35-Lu | A |
| 36-Lu | A |
| 37-Lu | A |
| 38-Lu | B |
| 39-Lu | A |
| 40-Lu | A |
| 41-Lu | A |
| 42-Lu | A |
| 43-Lu | A |
| 44-Lu | A |
| 45-Lu | A |
| 46-Lu | A |
| 47-Lu | A |
| 48-Lu | A |
| 49-Lu | A |
| 50-Lu | A |
| 51-Lu | A |
| 52-Lu | A |
| 53-Lu | A |
| 54-Lu | B |
| 55-Lu | A |
| 56-Lu | A |
| 57-Lu | A |
| 58-Lu | A |
| 59-Lu | A |
| 60-Lu | A |
| 61-Lu | A |
| 62-Lu | A |
| 63-Lu | A |
| 64-Lu | A |
| 65-Lu | A |
| 66-Lu | B |
| 67-Lu | A |
| 68-Lu | A |
| 69-Lu | A |
| 70-Lu | A |
| 71-Lu | A |
| 72-Lu | A |
| 73-Lu | A |
| 74-Lu | A |
| 75-Lu | A |
| 76-Lu | A |
| 77-Lu | A |
| 78-Lu | A |
| 79-Lu | B |
| 80-Lu | A |
| 81-Lu | A |
| 82-Lu | A |
| 83-Lu | A |
| 84-Lu | A |
| 85-Lu | A |
| 86-Lu | A |

We claim:

1. A compound of the formula 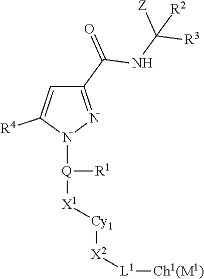
wherein,
R¹ is C₁-C₆ alkyl or C₃-C₆ cycloalkyl;
Z is —COOH, —C(═O)NH₂, or -tetrazolyl;
R² is C₆-C₁₀ cycloalkyl or C₆-C₁₀ cycloalkyl-C₁-C₆ alkyl-, wherein the C₆-C₁₀ cycloalkyl and C₆-C₁₀ cycloalkyl-C₁-C₆ alkyl- are each optionally substituted with 1-3 halogen;
R³ is H; or
R² and R³ together with the carbon atom to which they are attached form a C₆-C₁₀ cycloalkyl optionally substituted with 1-3 halogen;
R⁴ is a group of the formula: 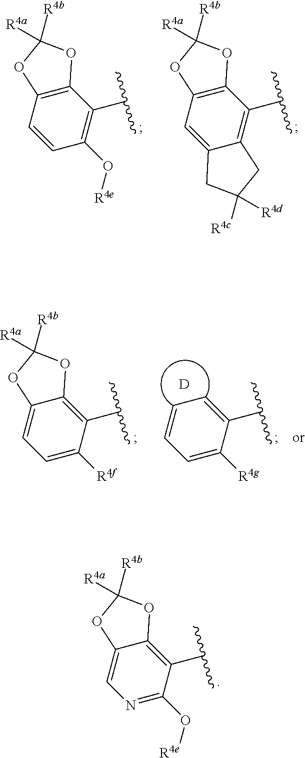
R^{4a}, R^{4b}, R^{4c}, and R^{4d} are each independently H or halogen;
R^{4e} is H, methyl, trifluoromethyl, or methoxyethyl;
R^{4f} is cyano, or fluoro;
R^{4g} is hydrogen;
D is a 5- to 6-membered heteroaryl containing 1-3 heteroatoms independently selected from N, O, and S, optionally substituted with 1-3 groups independently selected from halo, hydroxy, amino, cyano, nitro, C₁-C₆ alkyl, C₁-C₆ haloalkyl, C₁-C₆ alkoxy, C₁-C₆ haloalkoxy; C₁-C₆ alkylamino, and C₁-C₆ dialkylamino
Q is a 6-membered aryl or heteroaryl ring containing 0-3 ring heteroatoms independently selected from N, O, and S, the remaining ring atoms being carbon;
X¹ is a bond, —O—, or —NR^{x1}—;
Cy¹ is a bond or a 4- to 12-membered monocyclic or polycyclic ring system optionally substituted with 1-3 substituents independently selected from halo, hydroxy, amino, cyano, nitro, C₁-C₆ alkyl, C₁-C₆ haloalkyl, C₁-C₆ alkoxy, C₁-C₆ haloalkoxy, C₁-C₆ alkylamino, and C₁-C₆ dialkylamino, and optionally bearing 1-3 oxo substituents; provided that when Cy¹ is a bond, X¹ is a bond;
X² is a bond, —NR^{x2}—, —O—, —S—, —C(O)—, —C(S)—, —S(O)—, —S(O)₂—, —NR^{x3}C(O)—, —NR^{x4}C(S)—, —C(O)NR^{x5}—, —C(S)NR^{x6}—, —NR^{x7}C(O)NR^{x8}—, —NR^{x9}C(S)NR^{x10}—, —NR^{x11}C(O)O—, —NR^{x12}C(S)O—, —NR^{x13}C(O)S—, —OC(O)NR^{x14}—, —OC(S)NR^{x15}—, —SC(O)NR^{x16}—, —S(O)₂NR^{x17}—, —NR^{x18}S(O)₂—, or —NR^{x19}S(O)₂NR^{x20}—;
R^{x1} is H or C₁-C₃ alkyl;
R^{x2}, R^{x3}, R^{x4}, R^{x5}, R^{x6}, R^{x7}, R^{x8}, R^{x9}, R^{x10}, R^{x11}, R^{x12}, R^{x13}, R^{x14}, R^{x15}, R^{x16}, R^{x17}, R^{x18}, R^{x19}, and R^{x20} are each independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
L¹ is a linker;
Ch¹ is a chelator; and
M¹ is absent or is a radionuclide complexed with Ch¹;
or a pharmaceutically acceptable salt thereof.

2. The compound of claim 1, or a pharmaceutically acceptable salt thereof, wherein, R⁴ is a group of the formula: 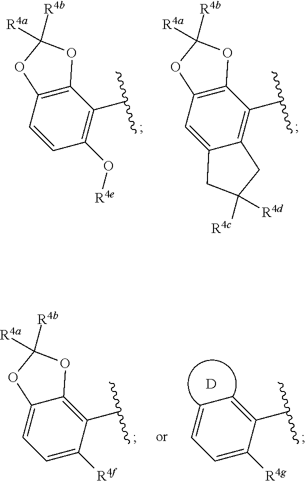
and
Q is

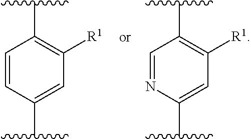

3. The compound of claim 1, or a pharmaceutically acceptable salt thereof, wherein
Z is —COOH,
R⁴ is a group of the formula: 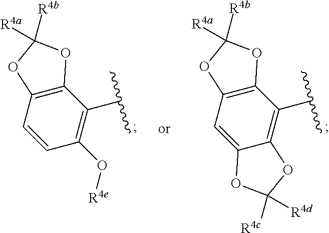
R^{4e} is methyl,
Cy¹ is phenyl or a 5- or 6-membered heteroaryl containing 1-3 heteroatoms independently selected from N, O, and S (provided that when Cy¹ is a bond, X¹ is a bond),
or a pharmaceutically acceptable salt thereof.

4. The compound of claim 1, or a pharmaceutically acceptable salt thereof, wherein R¹ is isopropyl.

5. The compound of claim 1, or a pharmaceutically acceptable salt thereof, wherein R¹ is cyclopropyl.

6. The compound of claim 1, or a pharmaceutically acceptable salt thereof, wherein R² is adamantyl or adamantyl-CH₂—.

7. The compound of claim 1, or a pharmaceutically acceptable salt thereof, wherein R² and R³ together with the carbon atom to which they are attached form an adamantyl.

8. The compound of claim 1, or a pharmaceutically acceptable salt thereof, wherein R⁴ is:

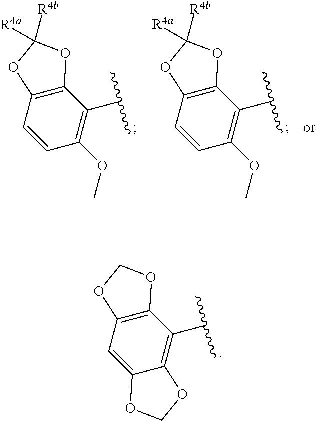

9. The compound of claim 1, or a pharmaceutically acceptable salt thereof, wherein R⁴ is:

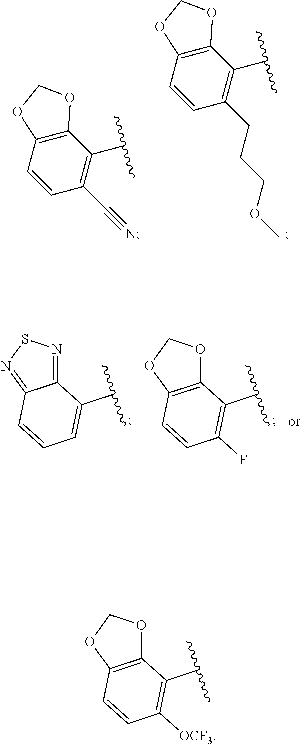

10. The compound of claim 1, or a pharmaceutically acceptable salt thereof, wherein R⁴ is:

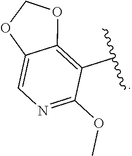 [SMILES: *c1c(OC)ncc2c1OCO2]

11. The compound of claim 1, or a pharmaceutically acceptable salt thereof, wherein Q is:

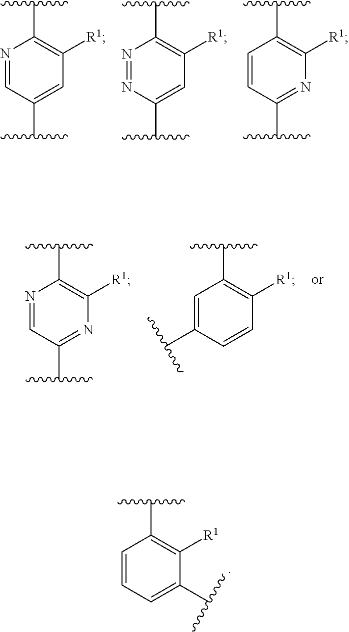

12. The compound of claim 1, or a pharmaceutically acceptable salt thereof, wherein X¹ is a bond.

13. The compound of claim 1, or a pharmaceutically acceptable salt thereof, wherein X¹ is —O—.

14. The compound of claim 1, or a pharmaceutically acceptable salt thereof, wherein X¹ is —NH—.

15. The compound of claim 1, wherein Cy¹ is a bond, phenyl, a 5- or 6-membered heteroaryl containing 1-3 heteroatoms independently selected from N, O, and S, a C₄-C₆ cycloalkyl, a C₄-C₆ cycloalkenyl, a 4- to 6-membered heterocycloalkyl containing 1-3 heteroatoms independently selected from N, O, and S, or a 4- to 6-membered heterocycloalkenyl containing 1-3 heteroatoms independently selected from N, O, and S; wherein the phenyl, heteroaryl, cycloalkyl, cycloalkenyl, heterocycloalkyl, or heterocycloalkenyl is optionally fused to a ring selected from phenyl, a 5- or 6-membered heteroaryl, a C₄-C₆ cycloalkyl, a C₄-C₆ cycloalkenyl, a 4- to 6-membered heterocycloalkyl containing 1-3 heteroatoms independently selected from N, O, and S, and a 4- to 6-membered heterocycloalkenyl; or wherein the cycloalkyl, cycloalkenyl, heterocycloalkyl or heterocycloalkenyl is optionally spiro-connected to another ring selected from a C₄-C₆ cycloalkyl, a C₄-C₆ cycloalkenyl, a 4- to 6-membered heterocycloalkyl containing 1-3 heteroatoms independently selected from N, O, and S, or a 4- to 6-membered heterocycloalkenyl containing 1-3 heteroatoms independently selected from N, O, and S; or wherein the cycloalkyl, cycloalkenyl, heterocycloalkyl or heterocycloalkenyl optionally comprises a bridging group having 1, 2, or 3 bridging atoms independently selected from N, O, and S; and wherein Cy¹ is optionally substituted with 1-3 substituents independently selected from the group consisting of halo, hydroxy, amino, cyano, nitro, C₁-C₆ alkyl, C₁-C₆ haloalkyl, C₁-C₆ alkoxy, C₁-C₆ haloalkoxy, C₁-C₆ alkylamino, and C₁-C₆ dialkylamino, and optionally bears 1-3 oxo substituents; provided that when Cy¹ is a bond, X¹ is a bond.

16. The compound of claim 1, or a pharmaceutically acceptable salt thereof, wherein Cy¹ is a bond.

17. The compound of claim 1, or a pharmaceutically acceptable salt thereof, wherein Cy¹ is phenyl.

18. The compound of claim 1, or a pharmaceutically acceptable salt thereof, wherein Cy¹ is triazolyl.

19. The compound of claim 1, or a pharmaceutically acceptable salt thereof, wherein Cy¹ is

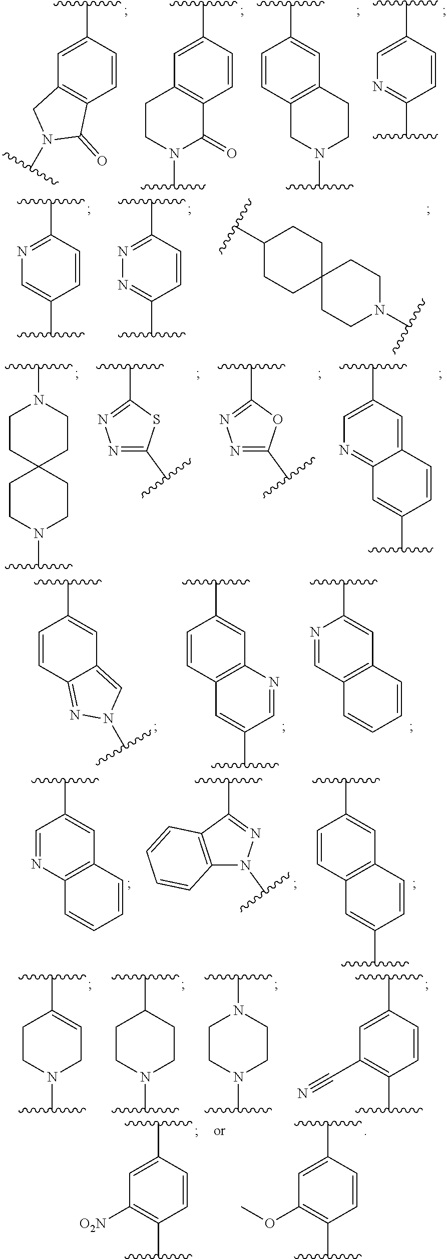

20. The compound of claim 1, or a pharmaceutically acceptable salt thereof, wherein Cy¹ is

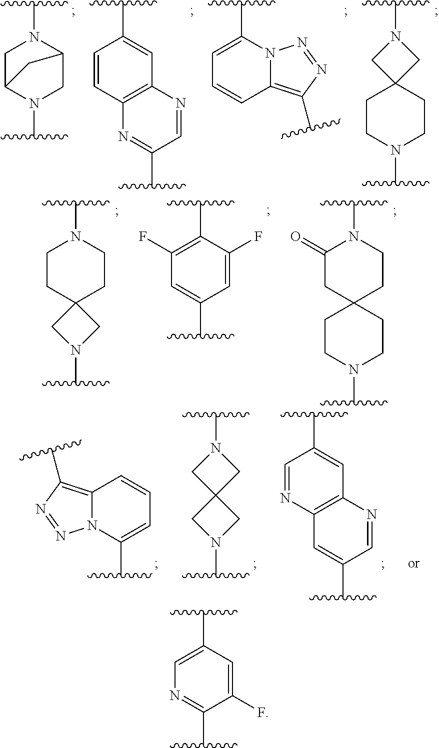

21. The compound of claim 1, or a pharmaceutically acceptable salt thereof, wherein X² is a bond, —NH—, —NCH₃—, —O—, —NHC(O)—, —NCH₃C(O)—, —C(O)NH—, —C(O)NCH₃—, —NHC(O)NH—, —NHC(O)NCH₃—, —NCH₃C(O)NCH₃—, —NHC(O)O—, —NCH₃C(O)O—, —NHC(O)S—, —NCH₃C(O)S—, —NHS(O)₂NH—, —NHS(O)₂NCH₃—, or —NCH₃S(O)₂NCH₃—.

22. The compound of claim 1, or a pharmaceutically acceptable salt thereof, wherein L¹ is 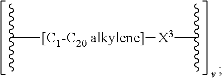
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}—, —C(S)NR^{x25}—, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}—, —OC(S)NR^{x34}—, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₂₀ alkylene at each occurrence is optionally interrupted by 1-5 groups independently selected from C₃-C₁₀ cycloalkylene, C₄-C₁₀ heterocycle, and C₆-C₁₀ arylene;
C₁-C₂₀ alkylene, C₃-C₁₀ cycloalkylene, C₄-C₁₀ heterocycle, and C₆-C₁₀ arylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl); and
v is 1, 2, 3, 4, or 5.

23. The compound of claim 22, or a pharmaceutically acceptable salt thereof, wherein L¹ is 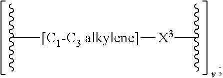
X³ at each occurrence is independently selected from —NR^{x21}—, —O—, —S—, —C(O)—, —C(S)—, —S(O)—, —S(O)₂—, —NR^{x22}C(O)—, —NR^{x23}C(S)—, —C(O)NR^{x24}, —C(S)NR^{x25}, —NR^{x26}C(O)NR^{x27}—, —NR^{x28}C(S)NR^{x29}—, —NR^{x30}C(O)O—, —NR^{x31}C(S)O—, —NR^{x32}C(O)S—, —OC(O)NR^{x33}, —OC(S)NR^{x34}, —SC(O)NR^{x35}—, —S(O)₂NR^{x36}—, —NR^{x37}S(O)₂—, or —NR^{x38}S(O)₂NR^{x39}—;
R^{x21}, R^{x22}, R^{x23}, R^{x24}, R^{x25}, R^{x26}, R^{x27}, R^{x28}, R^{x29}, R^{x30}, R^{x31}, R^{x32}, R^{x33}, R^{x34}, R^{x35}, R^{x36}, R^{x37}, R^{x38}, and R^{x39} at each occurrence are independently selected from H and C₁-C₆ alkyl, wherein the C₁-C₆ alkyl at each occurrence is optionally substituted with amino, C₁-C₆ alkylamino, C₁-C₆ dialkylamino, or a 5- or 6-membered heterocyclyl containing 1-3 heteroatoms independently selected from N, O, and S, wherein the heterocyclyl is optionally substituted with C₁-C₆ alkyl;
C₁-C₃ alkylene at each occurrence is optionally interrupted by 1-5 groups independently selected from C₃-C₁₀ cycloalkylene, C₄-C₁₀ heterocycle, and C₆-C₁₀ arylene;
C₁-C₃ alkylene, C₃-C₁₀ cycloalkylene, C₄-C₁₀ heterocycle, and C₆-C₁₀ arylene at each occurrence are optionally substituted with 1-3 groups independently selected from halo, —CO₂H, —OH, —SH, —NH₂, —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl, wherein the alkyl groups of —NHC₁-C₄ alkyl, —N(C₁-C₄ alkyl)(C₁-C₄ alkyl), C₁-C₆ alkyl, —O—C₁-C₆ alkyl and —S—C₁-C₆ alkyl are each optionally substituted with 1-3 groups independently selected from halo, —CO₂H, —OH, —NH₂, —NHC₁-C₄ alkyl, and —N(C₁-C₄ alkyl)(C₁-C₄ alkyl); and
v is 1, 2, 3, 4, or 5.

24. The compound of claim 22, or a pharmaceutically acceptable salt thereof, wherein L¹ is 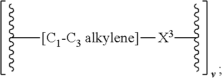
X³ at each occurrence is independently selected from —NH—, —NCH₃—, —O—, and —NHC(O)—; and
v is 1, 2, 3, 4, or 5.

25. The compound of claim 22, or a pharmaceutically acceptable salt thereof, wherein L¹ is

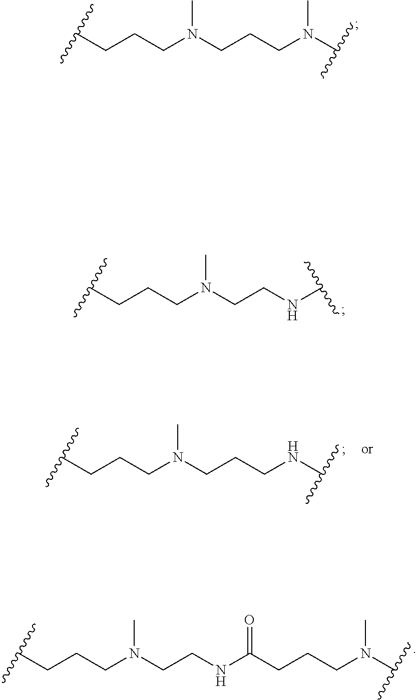

26. The compound of claim 22, or a pharmaceutically acceptable salt thereof, wherein L¹ is

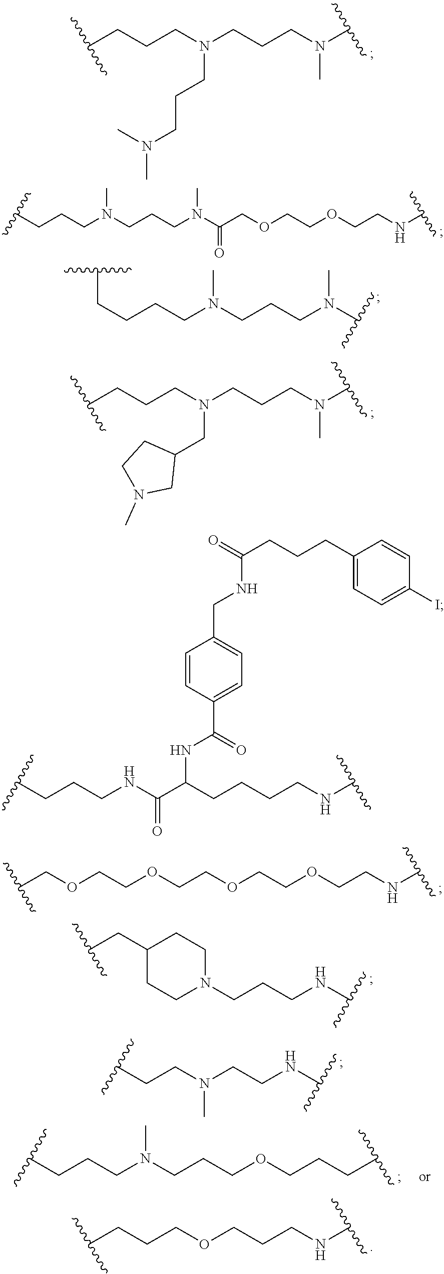

27. The compound of claim 22, or a pharmaceutically acceptable salt thereof, wherein L¹ is

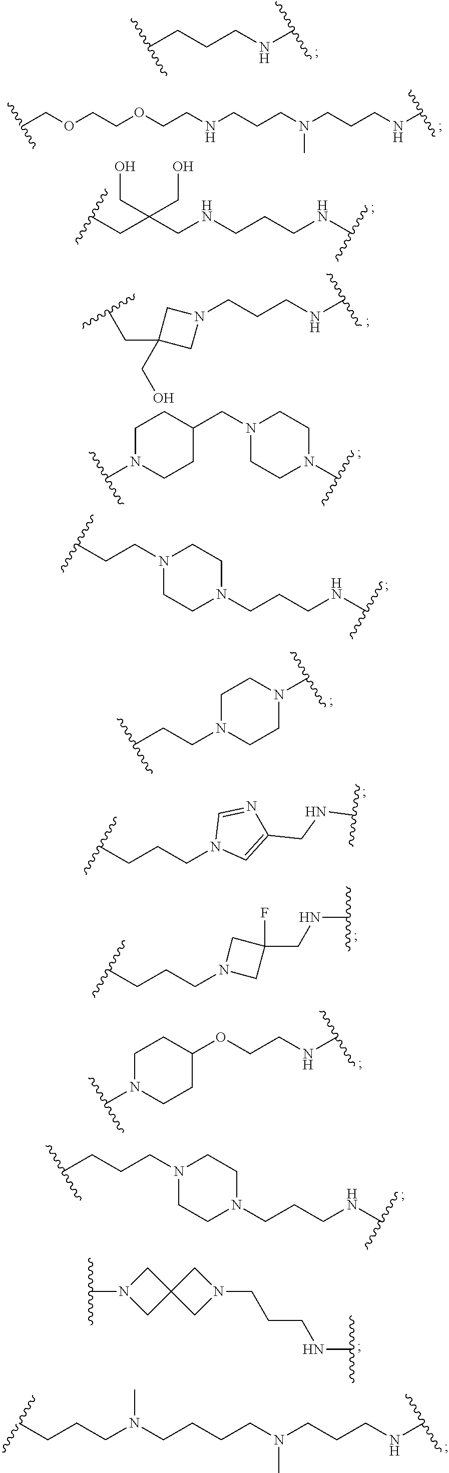

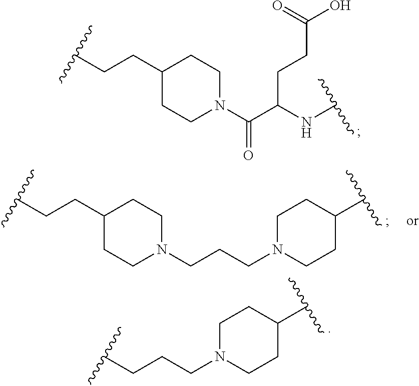

28. The compound of claim 1, or a pharmaceutically acceptable salt thereof, wherein Ch¹ is an aminopolycarboxylate chelator.

29. The compound of claim 1, or a pharmaceutically acceptable salt thereof, wherein Ch¹ is a macrocyclic aminopolycarboxylate chelator.

30. The compound of claim 1, or a pharmaceutically acceptable salt thereof, wherein Ch¹(M¹) is 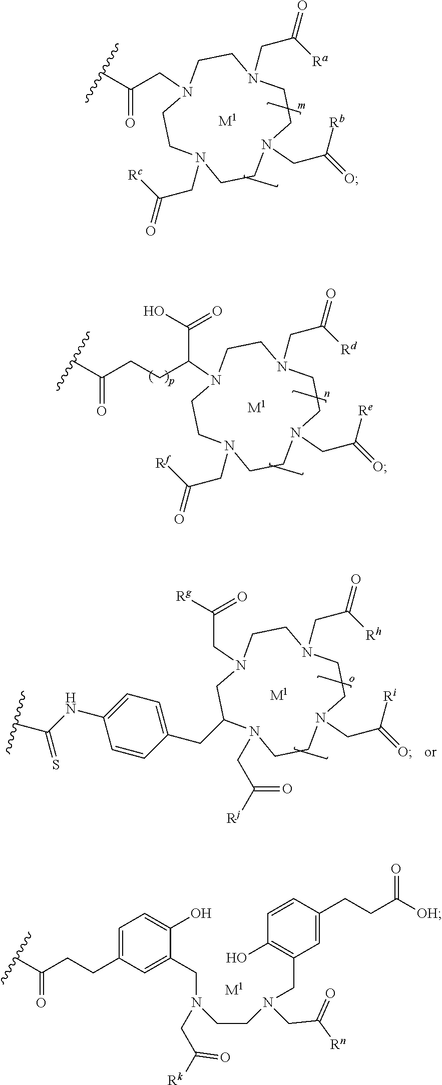
wherein R^{a}, R^{b}, R^{c}, R^{d}, R^{e}, R^{f}, R^{g}, R^{h}, Rⁱ, R^{j}, R^{k}, and Rⁿ are each independently selected from —OH and —NH₂; and m, n, o, and p are each independently 0, 1, or 2.

31. The compound of claim 30, or a pharmaceutically acceptable salt thereof, wherein Ch¹(M¹) is 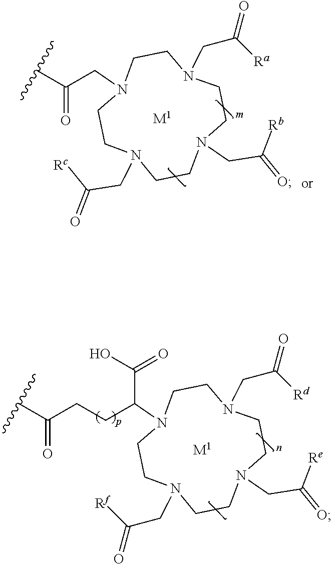
wherein R^{a}, R^{b}, R^{c}, R^{d}, R^{e}, and R^{f} are each independently selected from —OH and —NH₂; and m, n, and p are each independently 0, 1, or 2.

32. The compound of claim 30, or a pharmaceutically acceptable salt thereof, wherein Ch¹(M¹) is

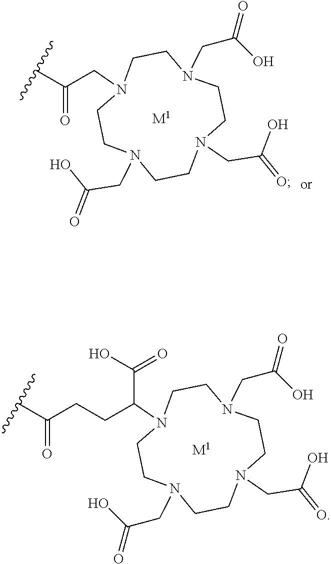

33. The compound of claim 1, or a pharmaceutically acceptable salt thereof, wherein M¹ is absent.

34. The compound of claim 1, or a pharmaceutically acceptable salt thereof, wherein M¹ is a radionuclide.

35. The compound of claim 34, or a pharmaceutically acceptable salt thereof, wherein M¹ is ¹⁷⁷Lu, ²¹²Pb, or ²²⁵Ac.

36. The compound of claim 34, or a pharmaceutically acceptable salt thereof, wherein M¹ is ¹⁷⁷Lu.

37. The compound of claim 34, or a pharmaceutically acceptable salt thereof, wherein M¹ is ⁶⁸Ga, ⁶⁴Cu, ⁸⁹Zr, ¹¹¹In, or an aluminum-fluoride complex of ¹⁸F.

38. The compound of claim 1, which is 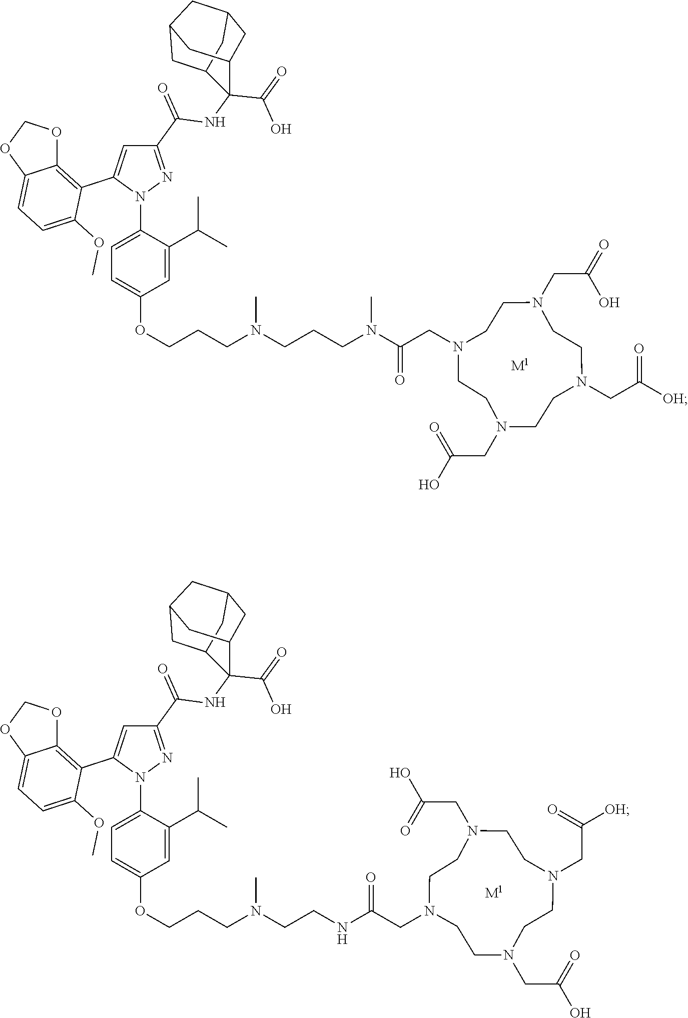 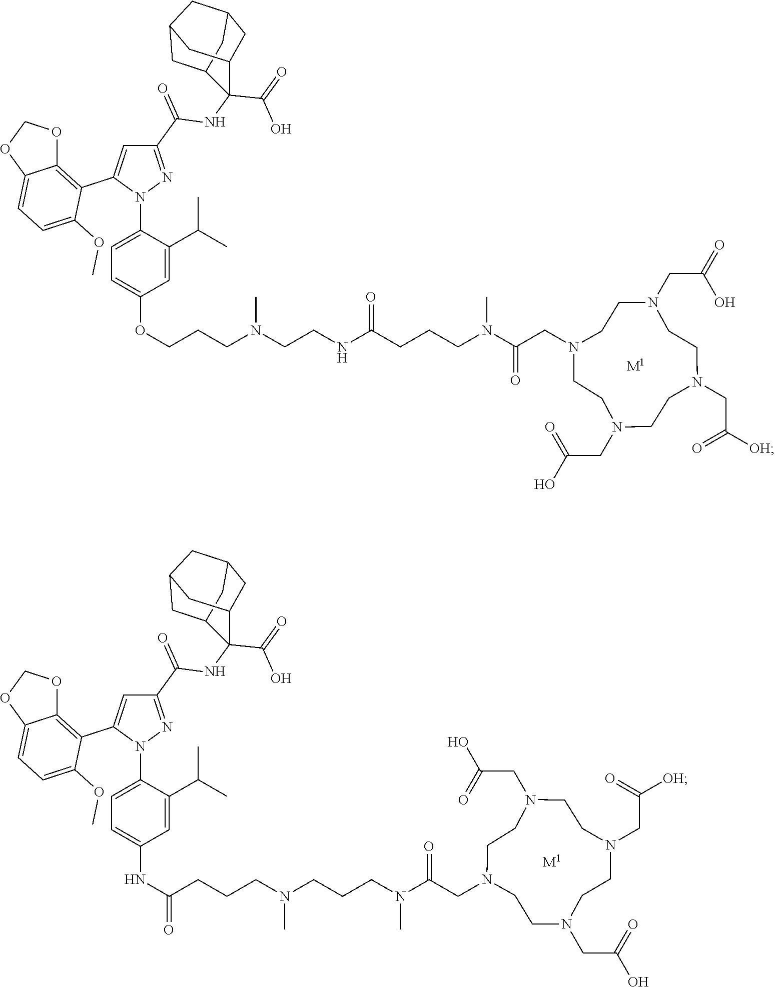 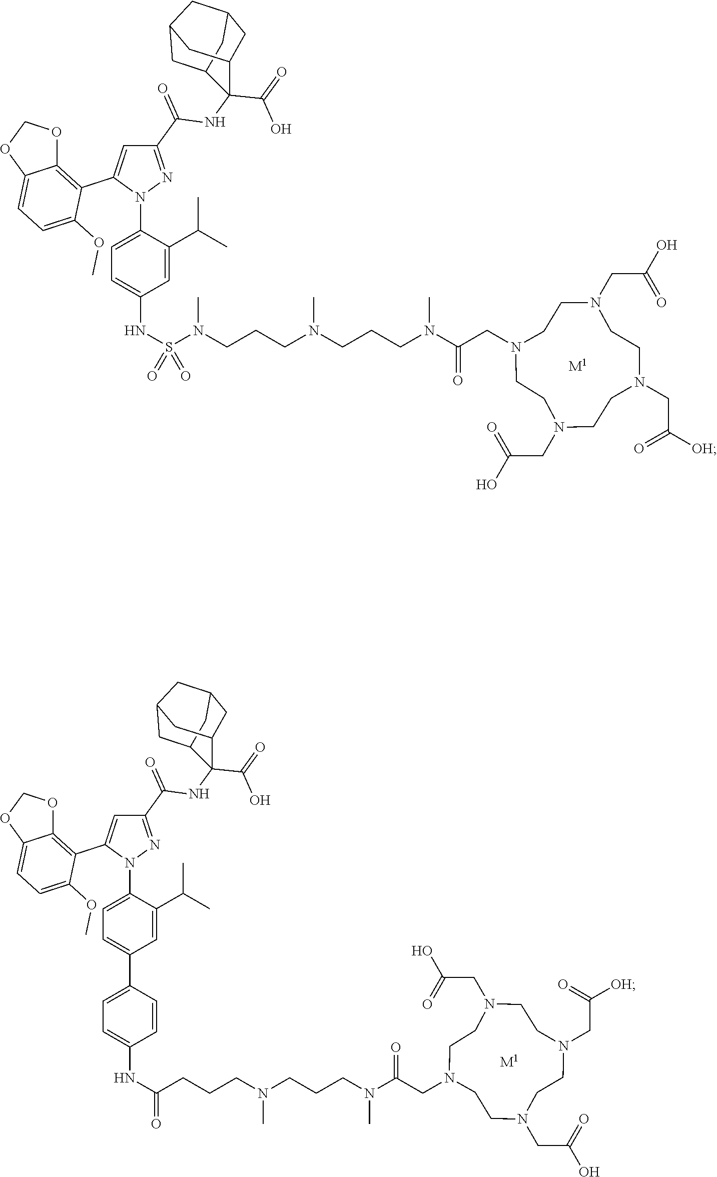 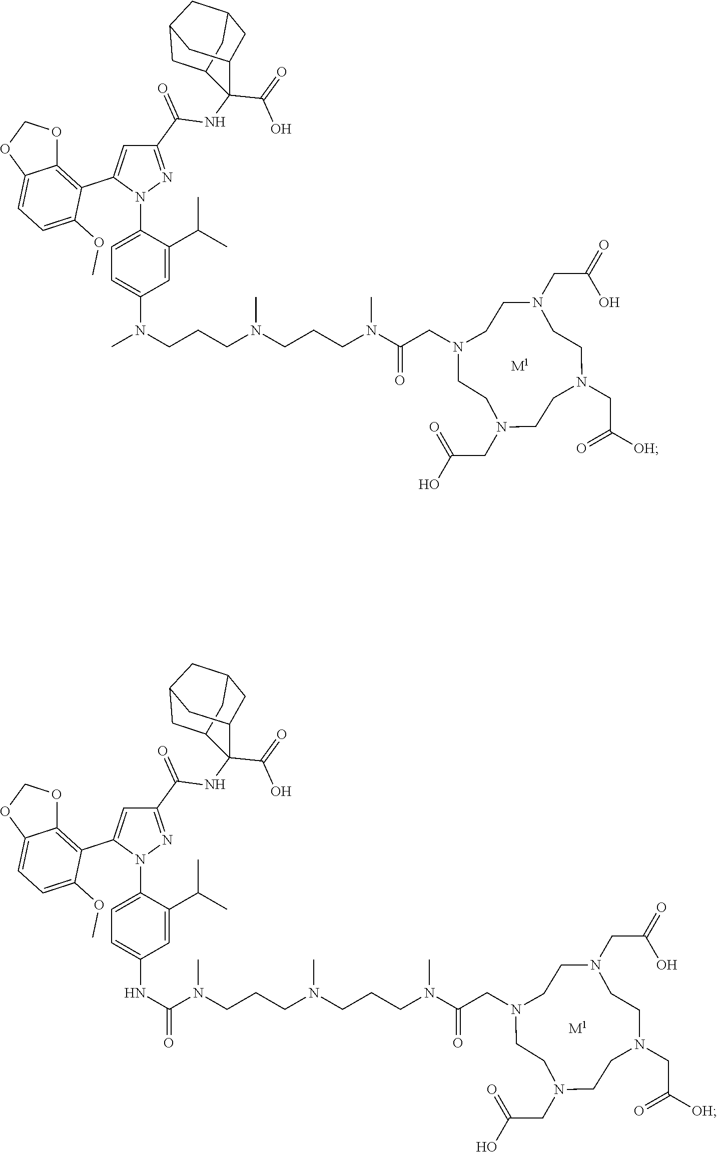 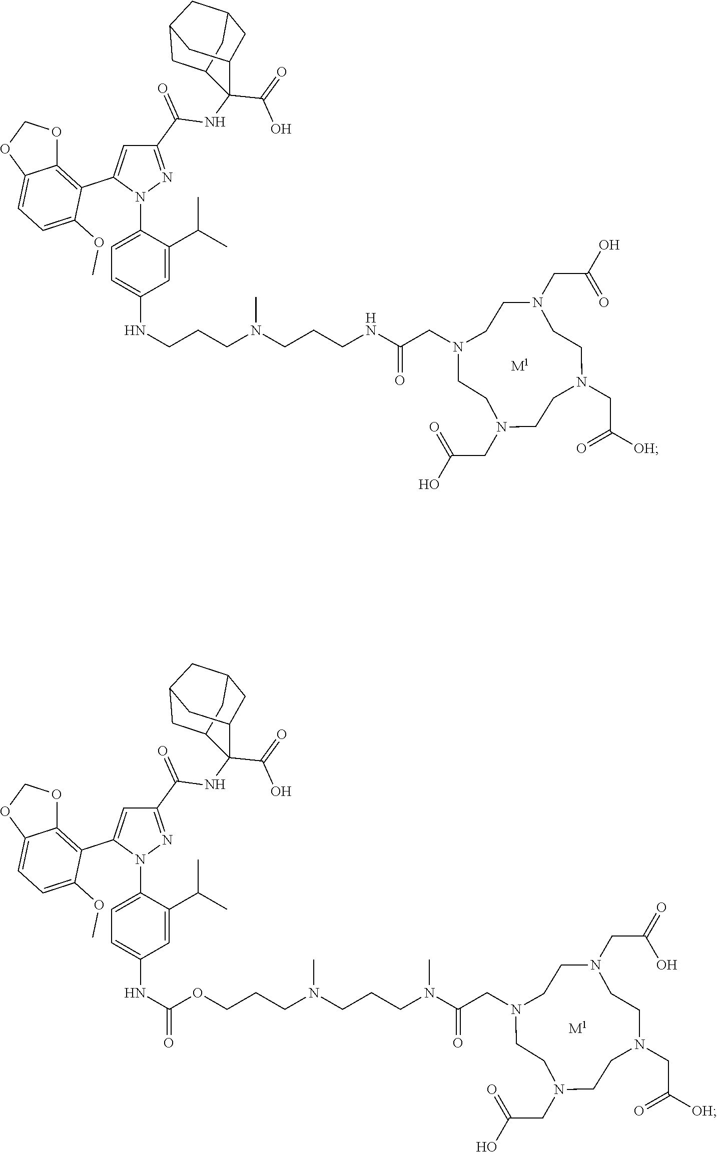 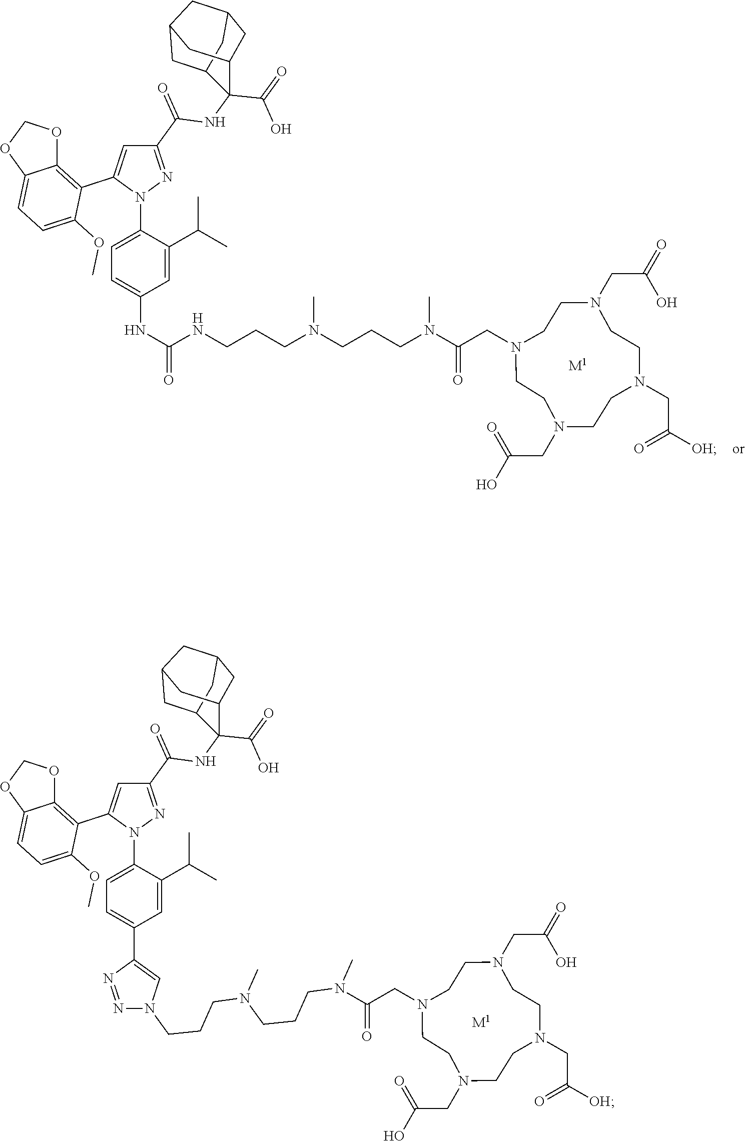
or a pharmaceutically acceptable salt thereof.

39. The compound of claim 1, which is

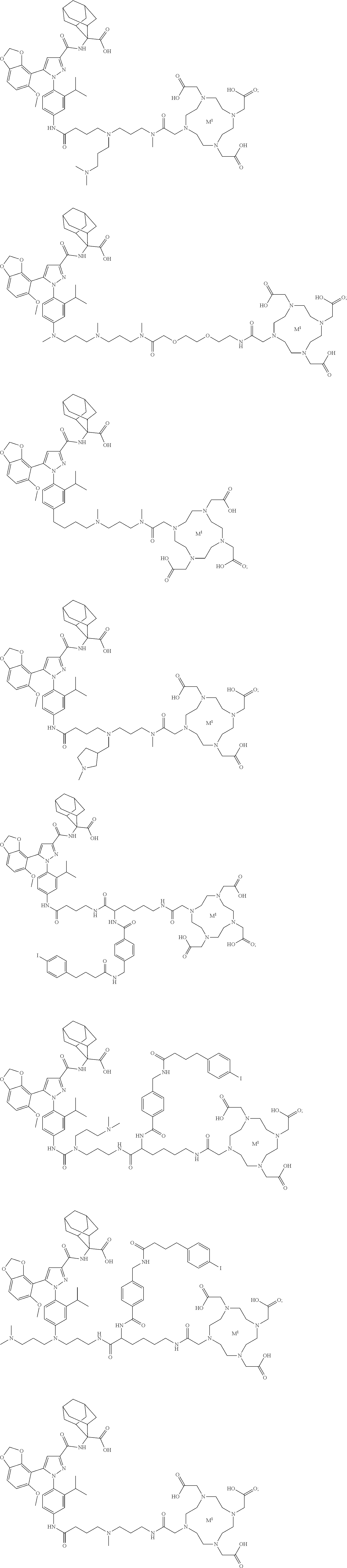

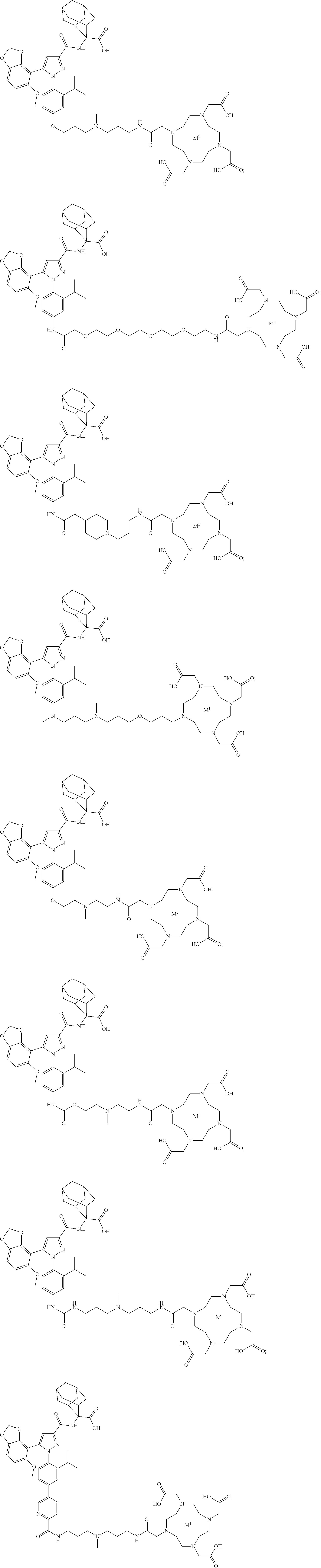

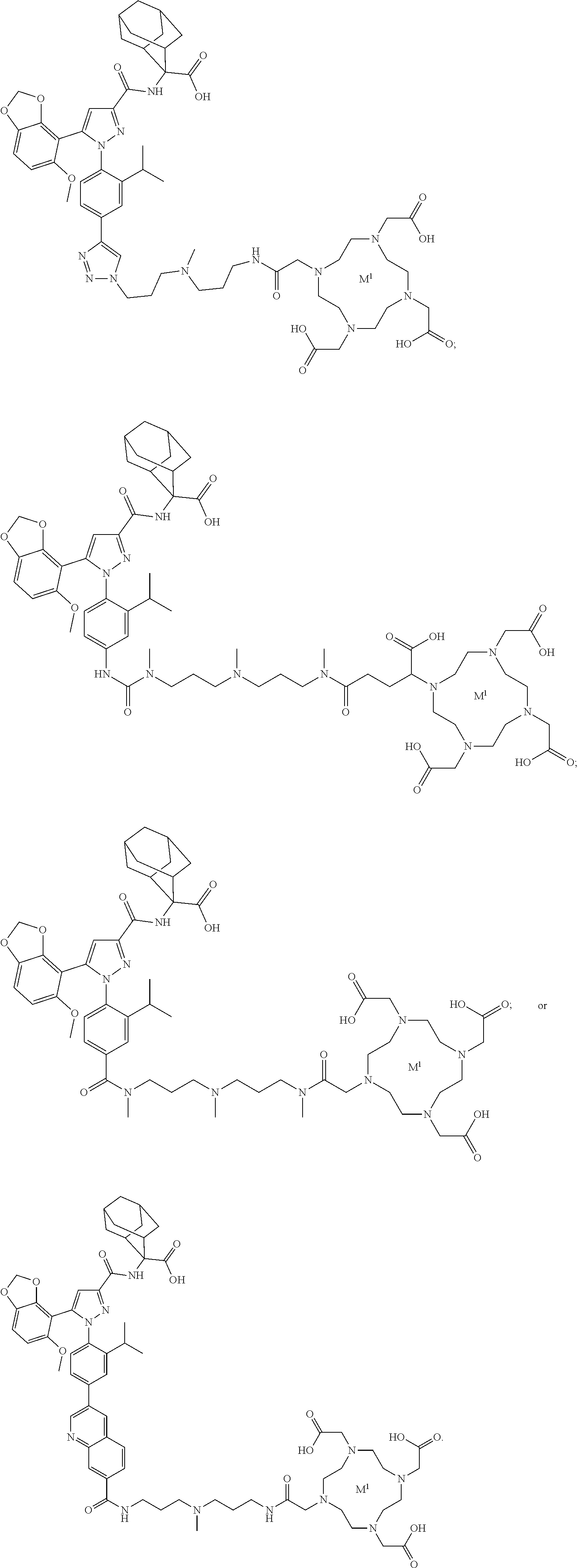

40. The compound of claim 1, which is

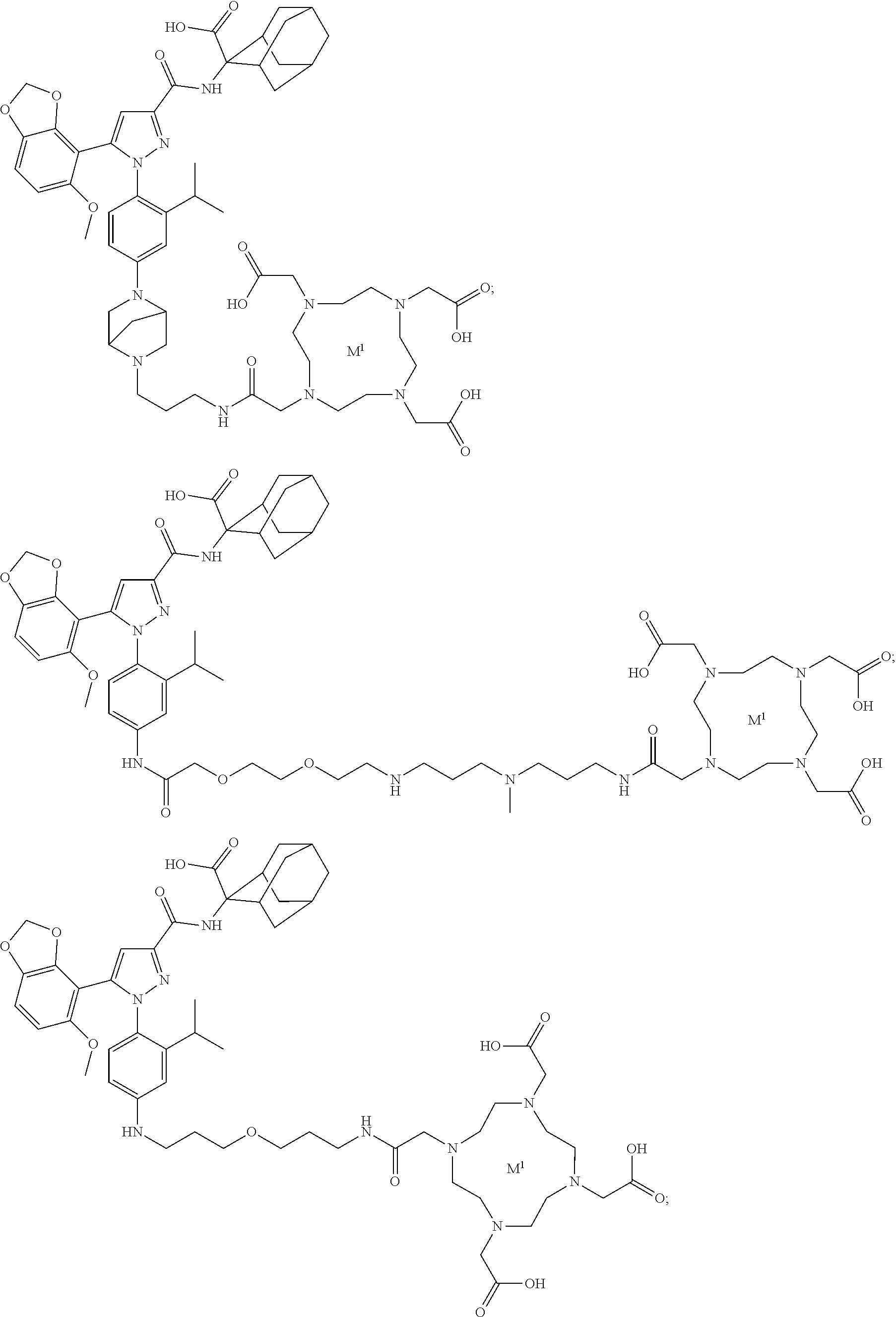

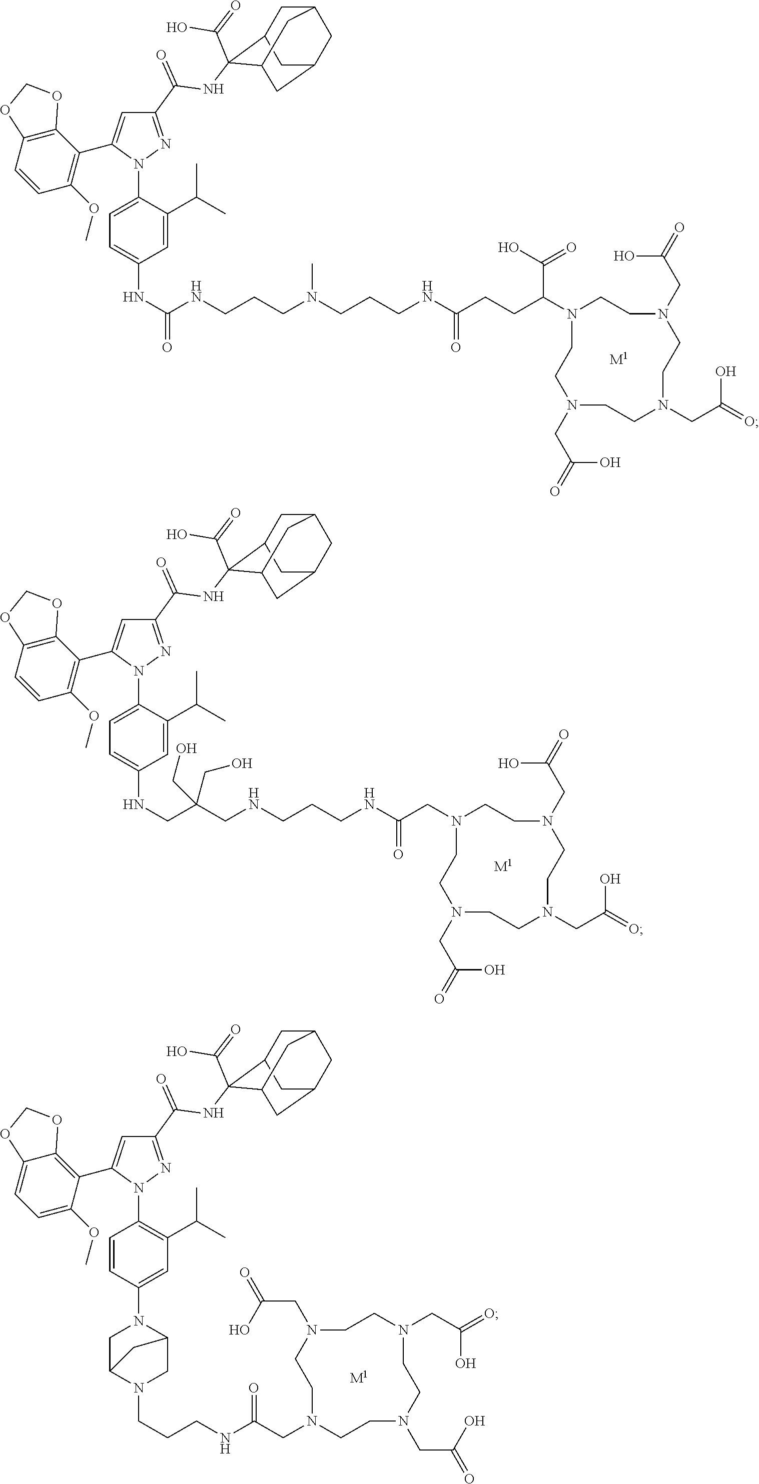

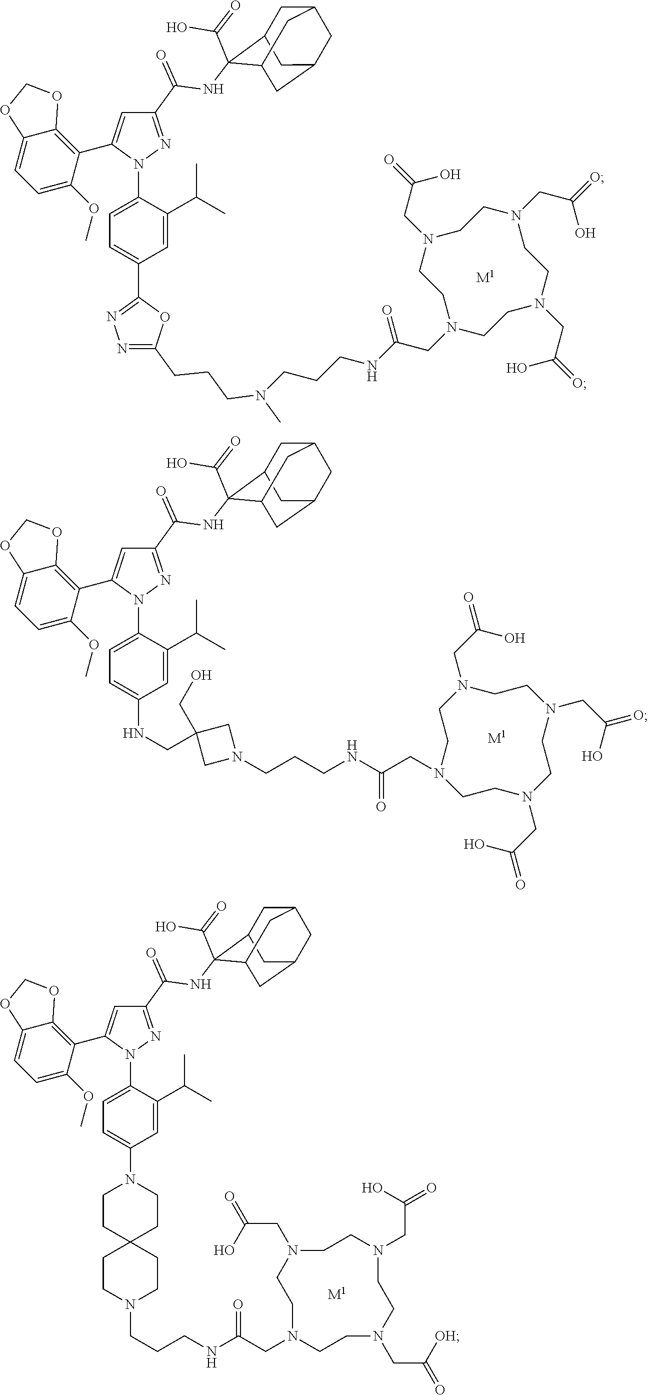

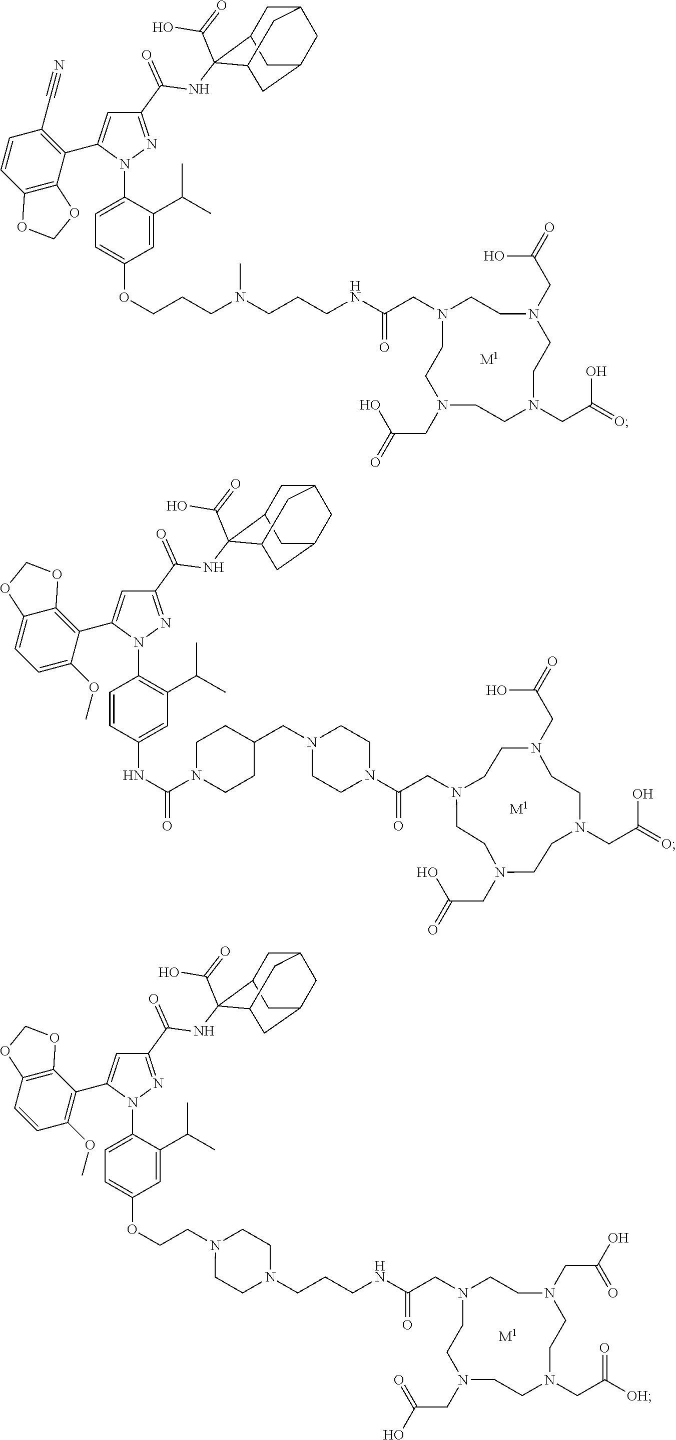

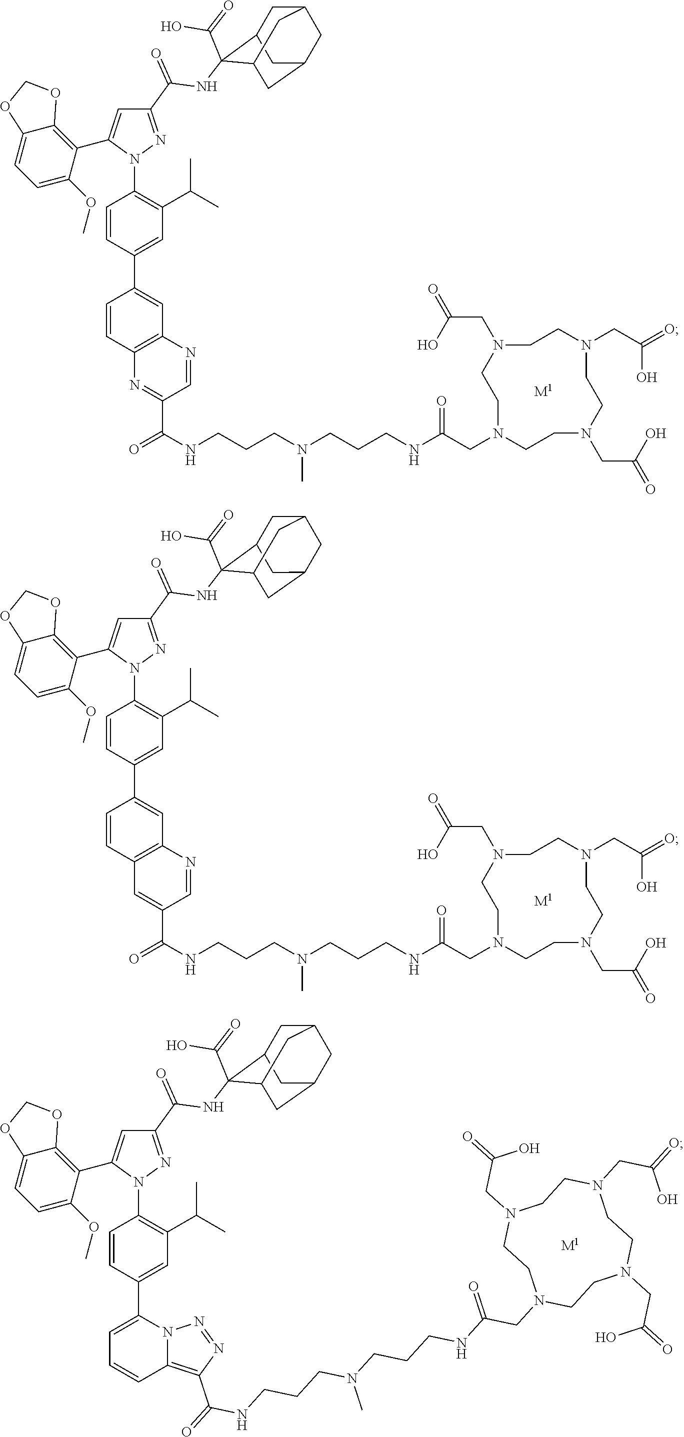

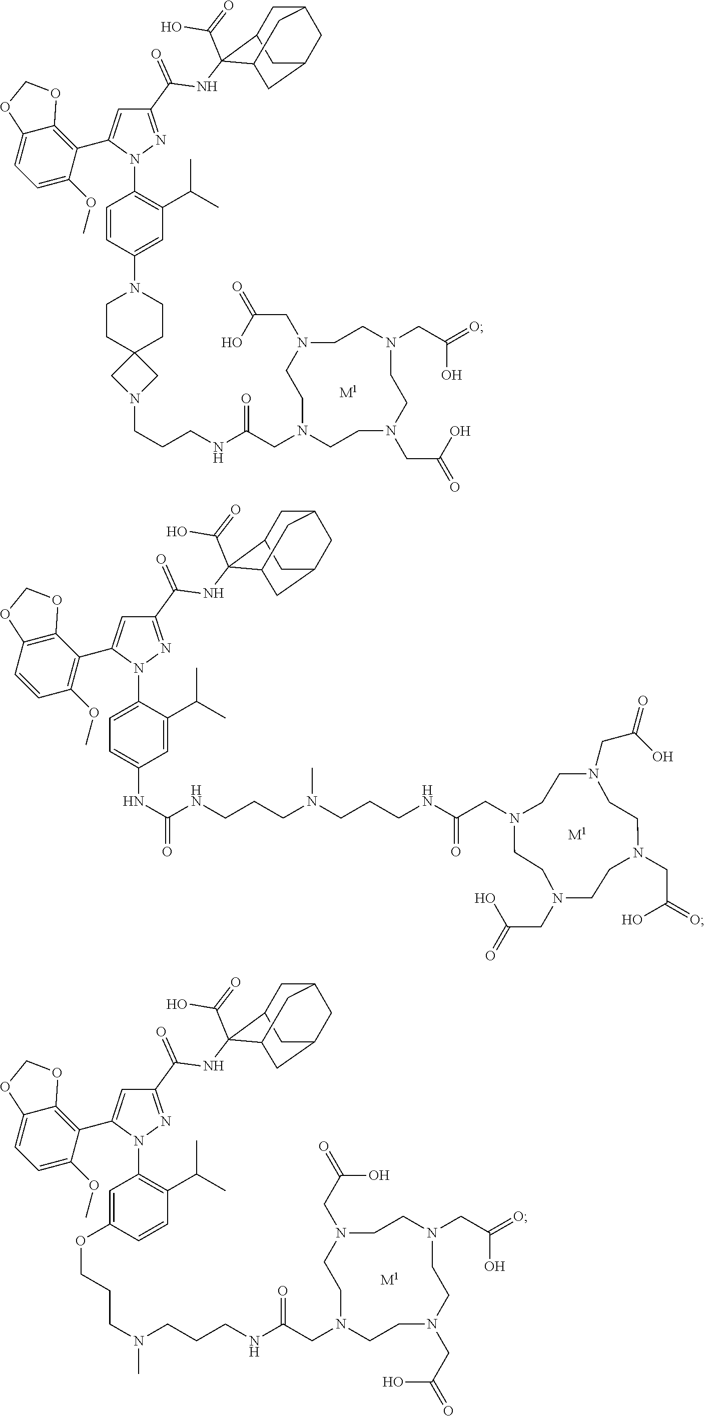

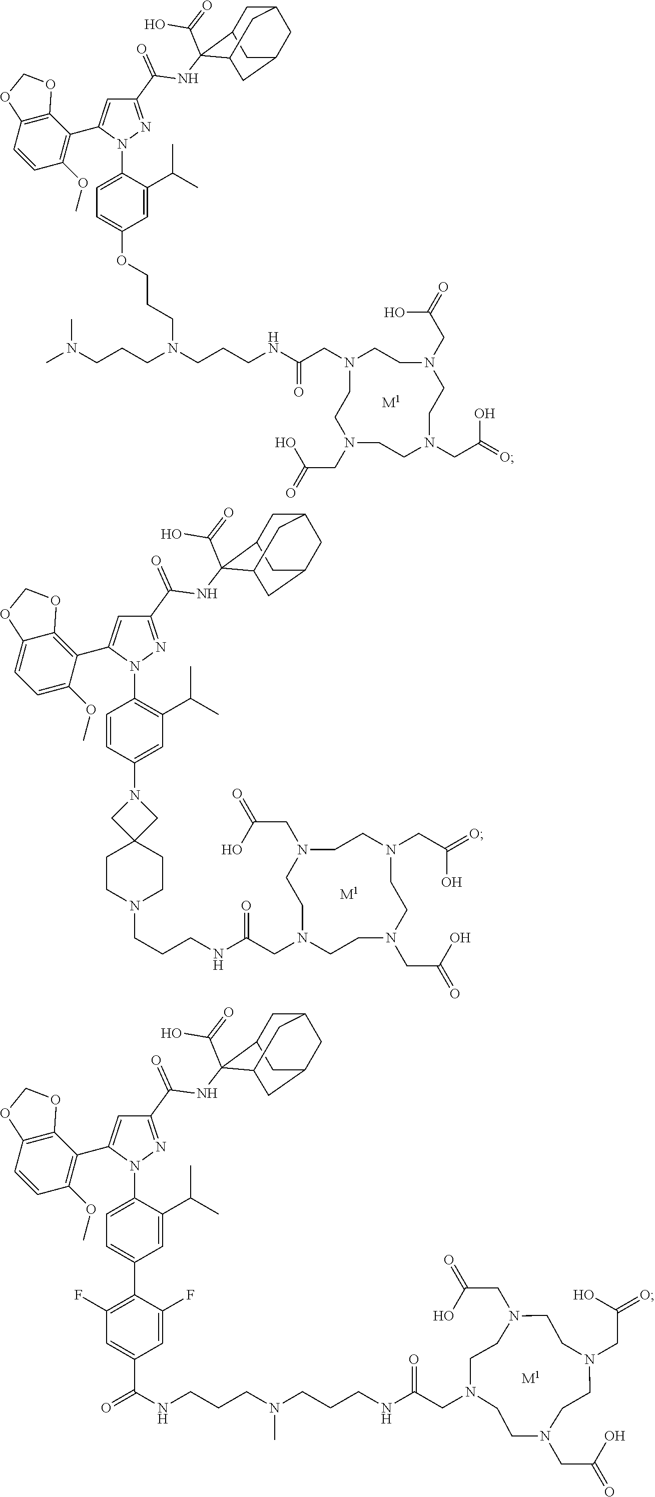

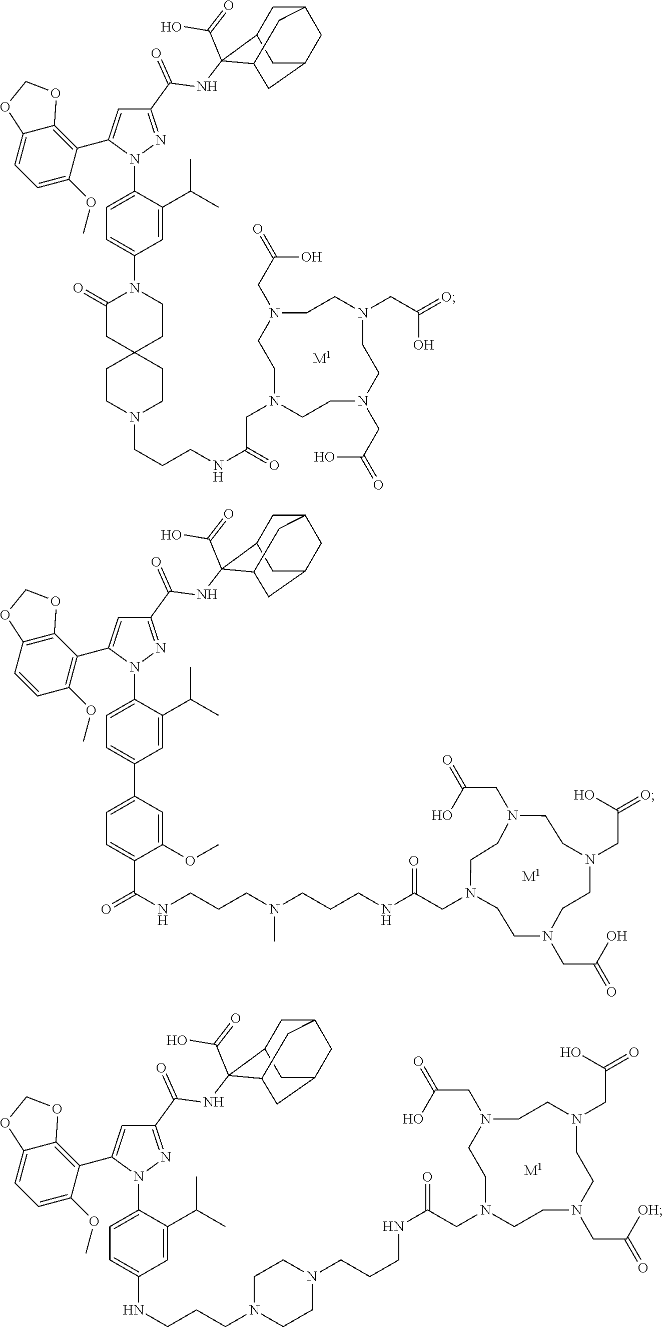

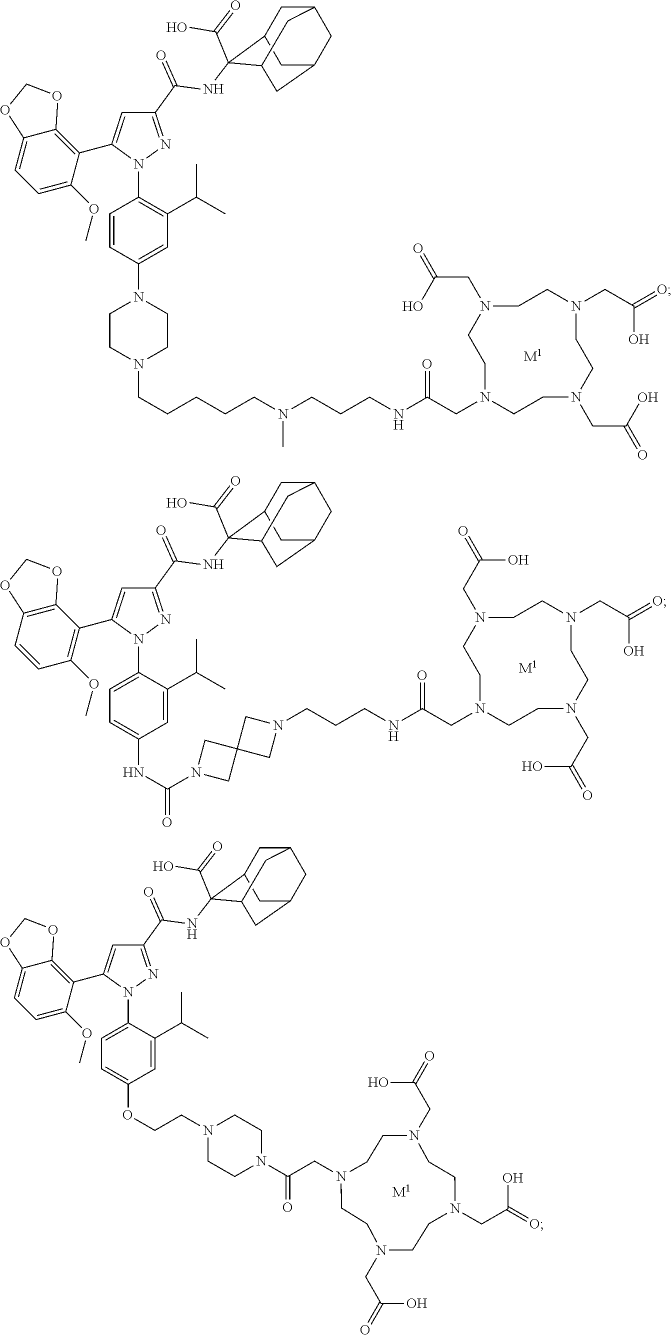

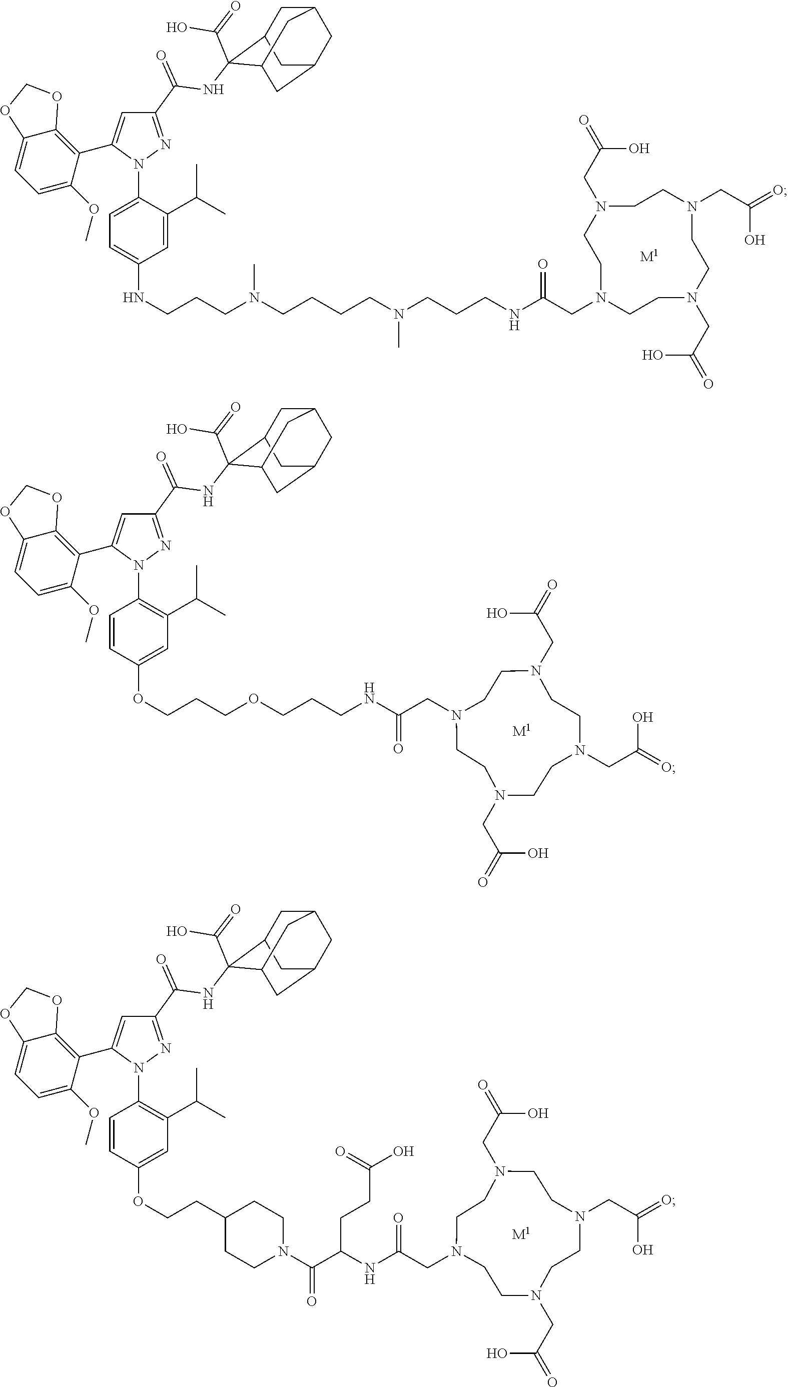

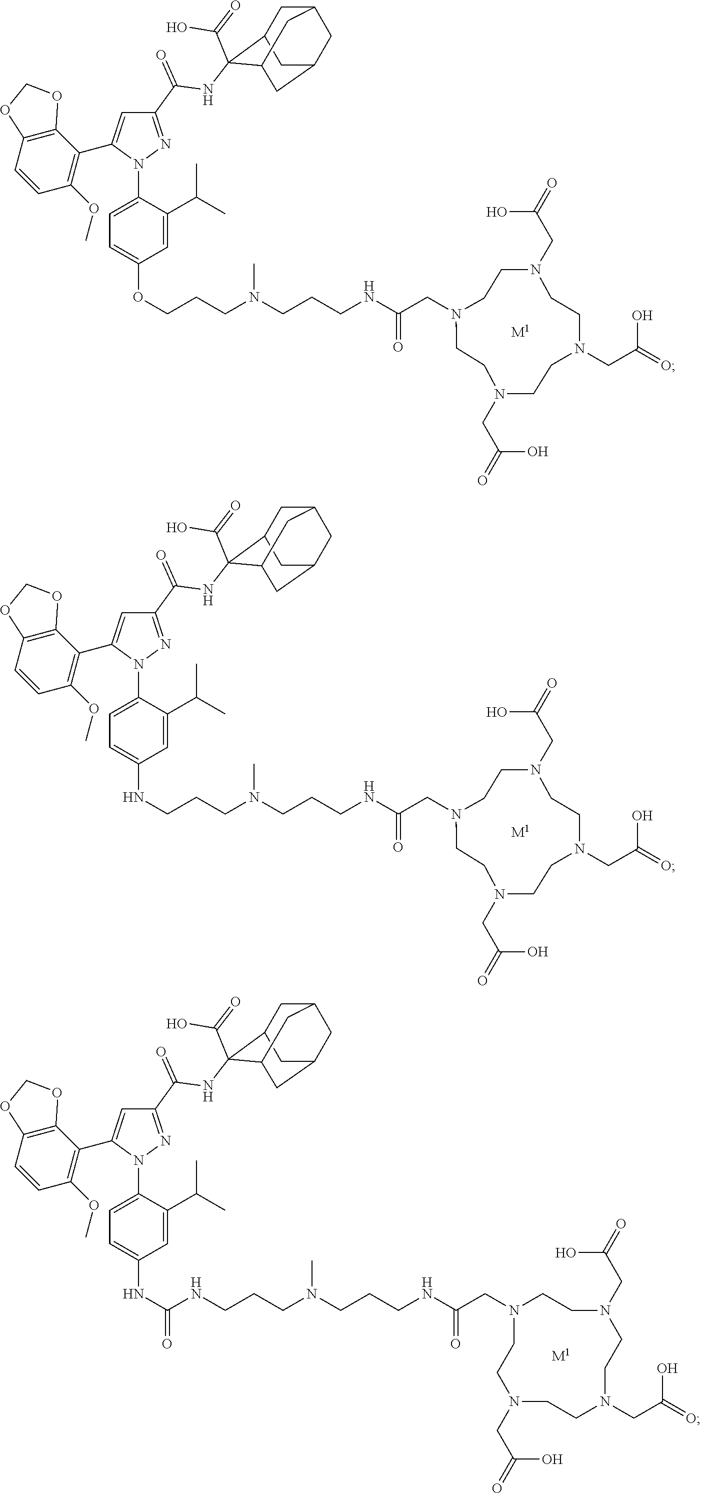

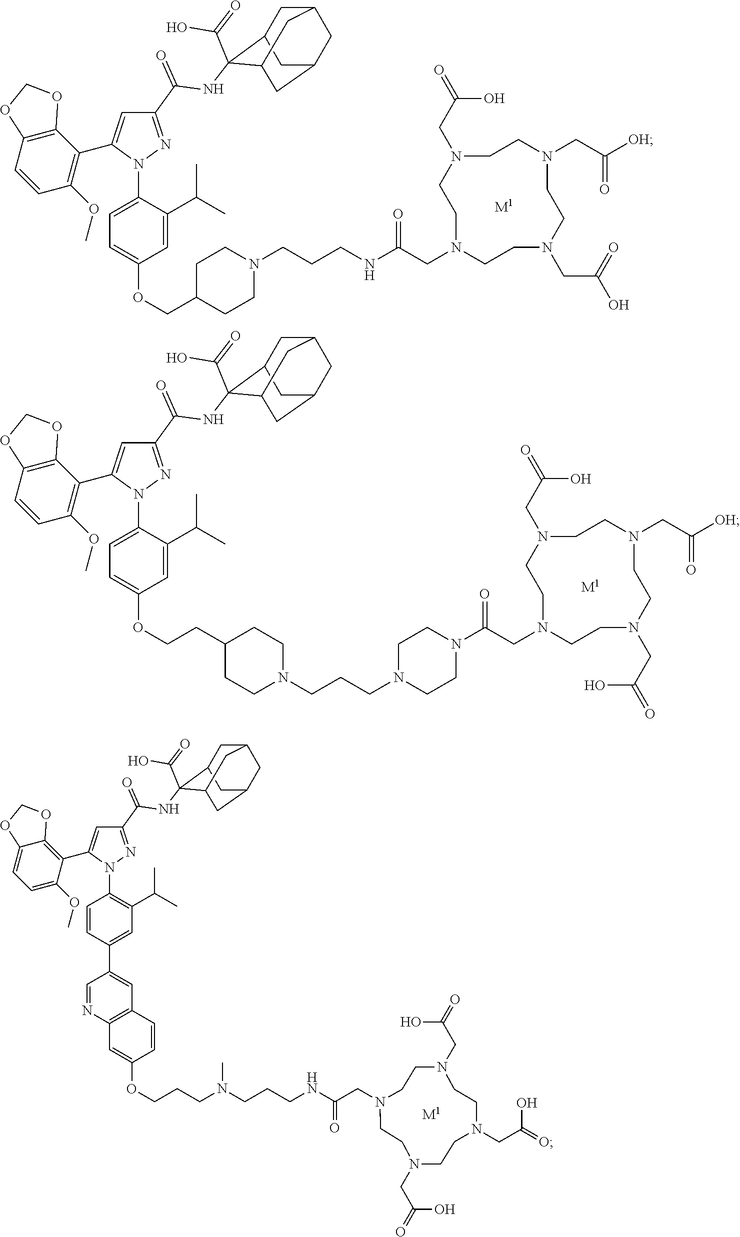

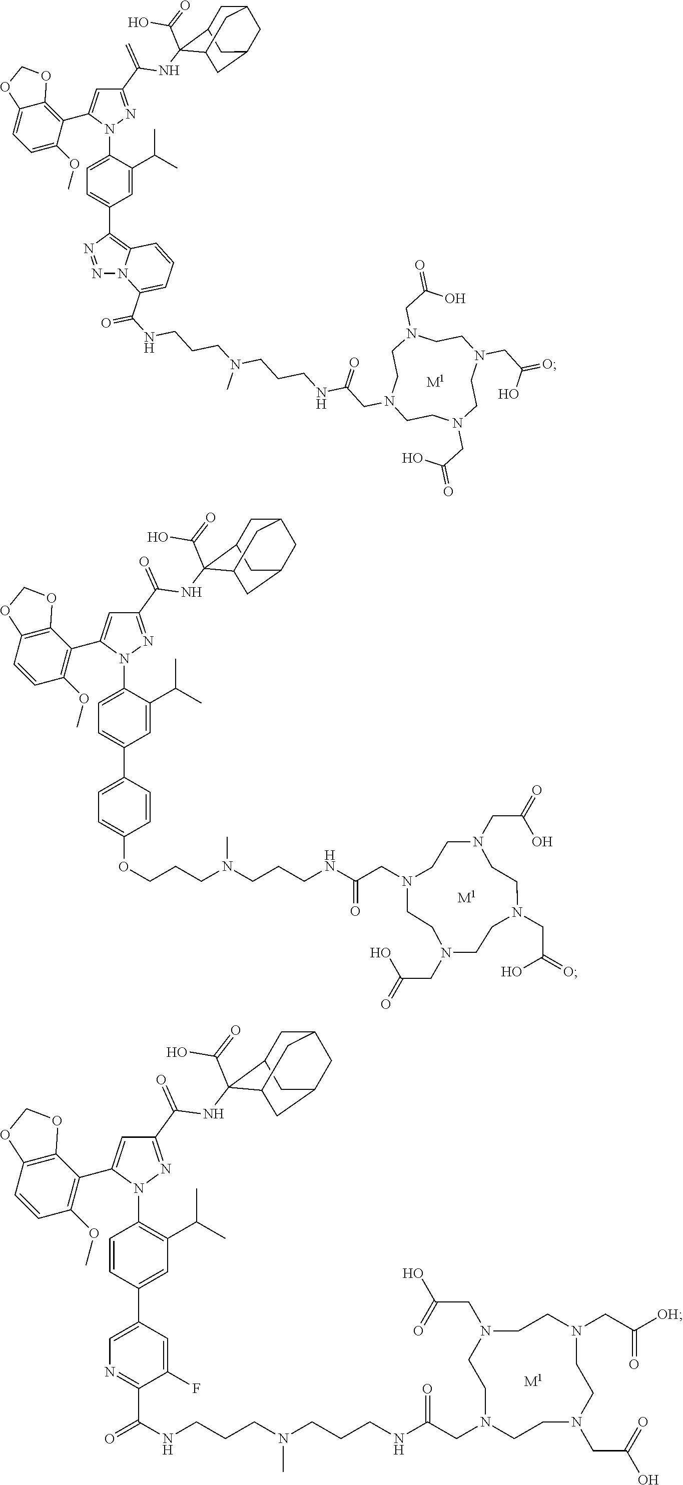

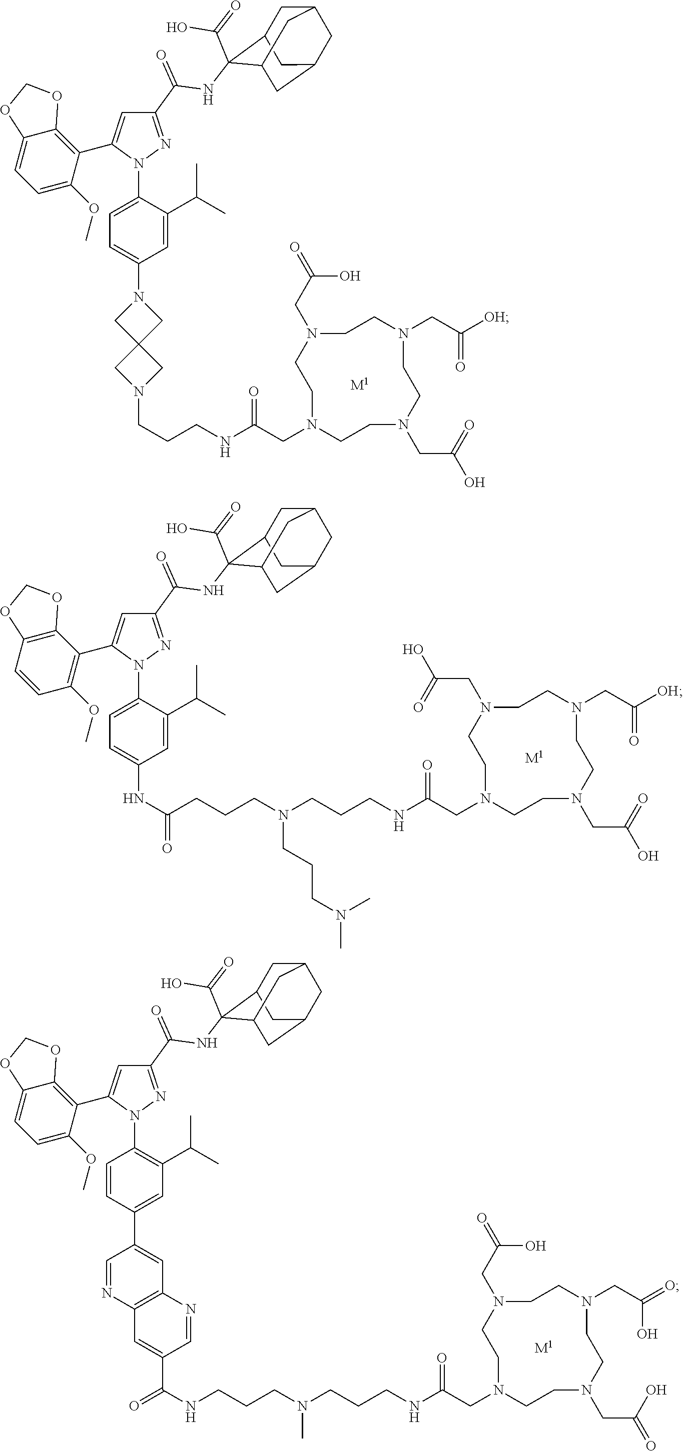

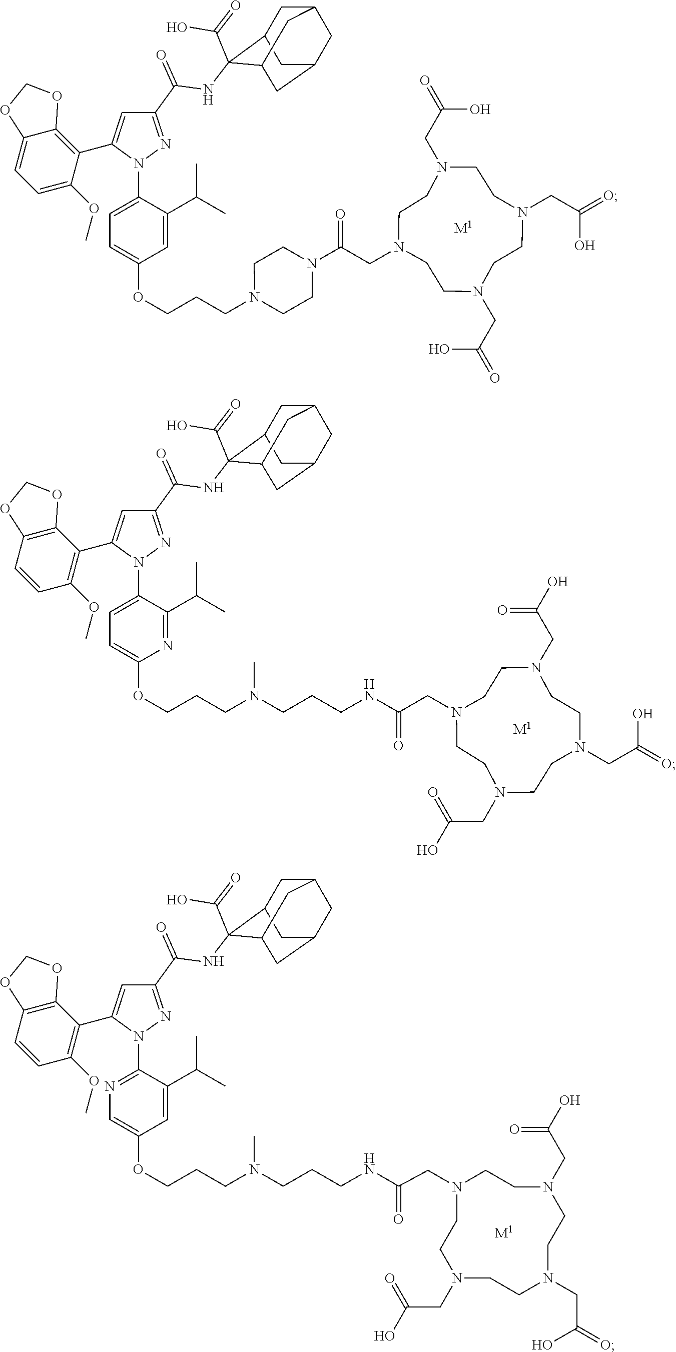

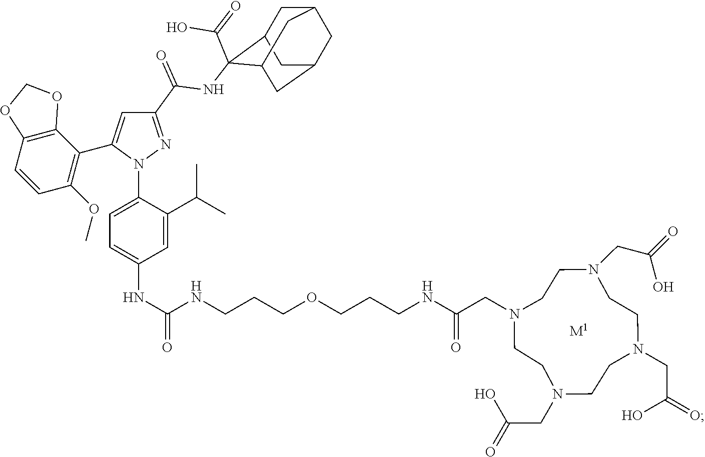

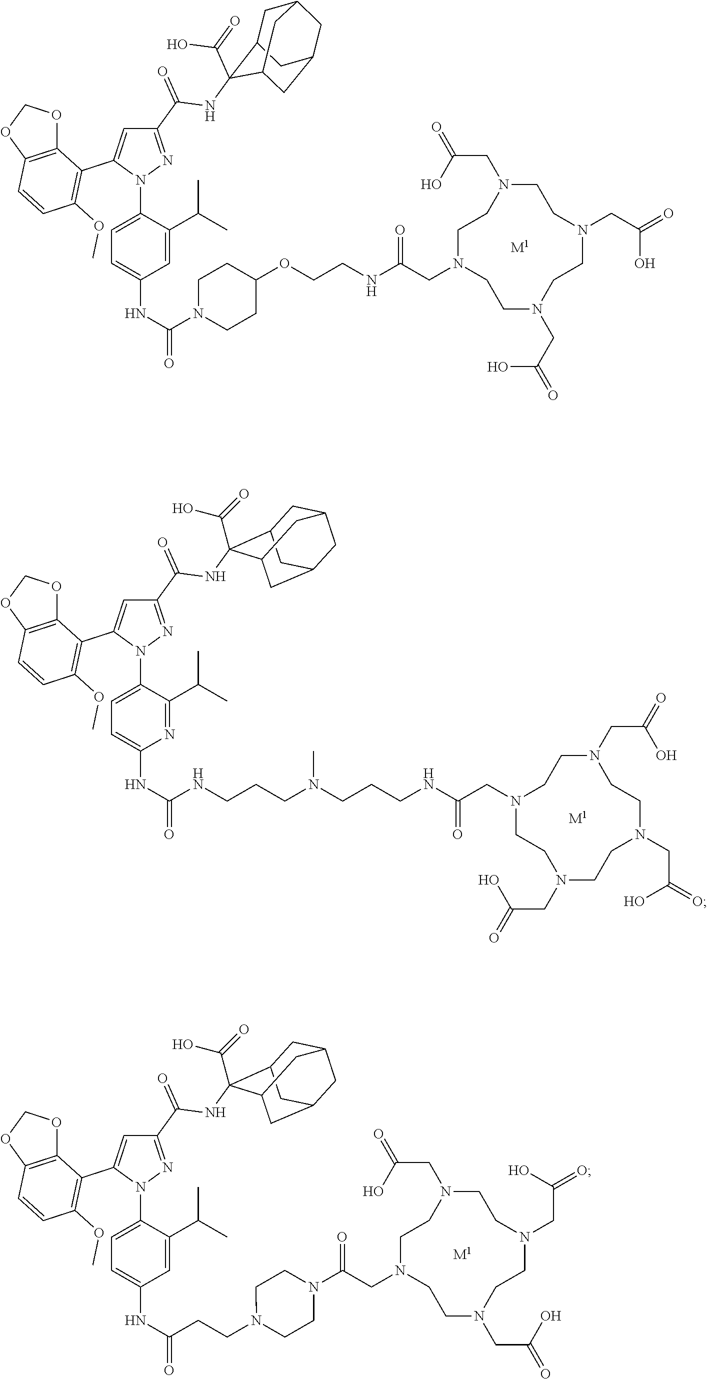

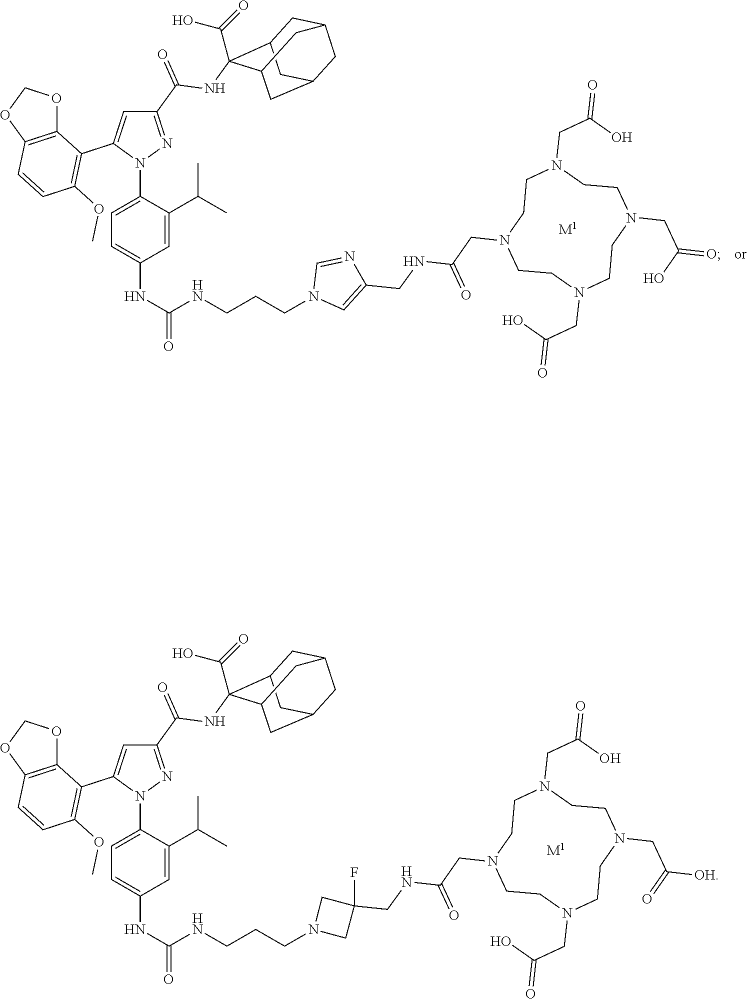

41. The compound of claim 1, or a pharmaceutically acceptable salt thereof, wherein M¹ is ¹⁷⁷Lu, ²¹²Pb, or ²²⁵Ac.

42. The compound of claim 1, or a pharmaceutically acceptable salt thereof, wherein M¹ is ¹⁷⁷Lu.

43. A pharmaceutical composition comprising a compound of claim 1, or a pharmaceutically acceptable salt thereof, and a pharmaceutically acceptable carrier, diluent or excipient.

44. A method of treating cancer, comprising administering to a patient in need thereof, a therapeutically effective amount of a compound of claim 1, or a pharmaceutically acceptable salt thereof.

45. The method of claim 44, wherein the cancer has one or more cancer cells that overexpress neurotensin receptor 1 (NTSR1).

46. The method of claim 44, wherein the cancer is breast cancer, colorectal cancer, endometrial cancer, gastric cancer, lung cancer, pancreatic cancer, prostate cancer, head and neck cancer, non-small-cell lung cancer (NSCLC), pleural mesothelioma, head and neck squamous cell carcinoma (HNSCC), glioma, glioblastoma multiforme (GBM), meningioma, Ewing's sarcoma, gastrointestinal stroma tumors, uterine leiomyoma, cutaneous T-cell lymphoma, small cell lung cancer, or pancreatic ductal adenocarcinoma.
